(19) United States
(12) Patent Application Publication
(10) Pub. No.: US 2024/0374738 A1
(43) Pub. Date: Nov. 14, 2024

(54) BIFUNCTIONAL COMPOUND CAPABLE OF DEGRADING RECEPTOR TYROSINE KINASE AND APPLICATION THEREOF

(71) Applicant: TAIBIDI PHARMACEUTICAL TECHNOLOGY (SHIJIAZHUANG) CO., LTD., Hebei (CN)

(72) Inventors: Xiangdong SU, Hebei (CN); Jinxu WANG, Hebei (CN); Fei QI, Hebei (CN); Chao XIN, Hebei (CN); Yakun GAO, Hebei (CN); Tianle WEN, Hebei (CN); Tingting GUO, Hebei (CN)

(21) Appl. No.: 18/751,042

(22) Filed: Jun. 21, 2024

(30) Foreign Application Priority Data
Mar. 16, 2023 (CN) 2023102574885

(51) Int. Cl. A61K 47/55; A61K 45/06; A61K 47/54

(52) U.S. Cl. CPC A61K 47/55; A61K 45/06; A61K 47/545

(57) ABSTRACT

The present disclosure belongs to the field of biomedicine, in particular to a bifunctional compound capable of degrading receptor tyrosine kinase or pharmaceutically acceptable salt, stereoisomer, solvate or polymorph thereof. This type of compound regulates downstream key proteins by degrading the receptor tyrosine kinase, especially discoidin domain receptors (DDRs), further plays a role in treating related diseases, and shows a prominent capability of inhibiting proliferation of tumor cells. A PROTAC compound developed by the present disclosure has a novel structure, excellent biological activity, and can safely and effectively inhibit or degrade a DDR1. The compound can effectively degrade or inhibit the receptor tyrosine kinase, especially the DDR1 and/or a DDR2, and can be used for treating diseases related to the DDR1 and other RTK homeostasis imbalances. The present disclosure further recruits a receptor tyrosine kinase target protein to specific E3 ligase and completes ubiquitination labeling and degradation and can be used for preparing a drug for prevention, diagnosis or treatment of receptor tyrosine kinase (RTK)-related diseases or symptoms.

### CROSS-REFERENCE TO RELATED APPLICATION

This application claims priority to Chinese Patent Application No. 2023102574885, filed Mar. 16, 2023, which is hereby incorporated by reference in its entirety.

### FIELD OF THE INVENTION

The present disclosure belongs to the field of biomedicine, in particular to a bifunctional compound capable of degrading receptor tyrosine kinase or pharmaceutically acceptable salt, stereoisomer, solvate or polymorph thereof. This type of compound regulates downstream key proteins by degrading the receptor tyrosine kinase, especially discoidin domain receptors (DDRs), further plays a role in treating related diseases, and shows a prominent capability of inhibiting proliferation of tumor cells.

### BACKGROUND OF THE INVENTION

Receptor tyrosine kinases (RTKs) are located on cell surfaces and are receptors having highly affinity for various signaling molecules such as growth factors, cytokines, and hormones. As an important component of a tyrosine kinase family, the receptor tyrosine kinases participate in cellular signal transduction by catalyzing downstream protein tyrosine phosphorylation. There are currently over fifty known RTKs, which may be divided into twenty different subfamilies, including an epidermal growth factor receptor subfamily, a vascular endothelial growth factor receptor subfamily, a hepatocyte growth factor receptor subfamily and the like. They play a very important role in a process of controlling cell proliferation, migration, differentiation and metabolism, respectively. In terms of a protein structure, the RTKs are composed of three components, including an extracellular domain (ECD) of ligand binding sites, a hydrophobic alpha helix transmembrane domain, and an intracellular domain containing tyrosine protein kinase activity. The intracellular domain may be further divided into a near membrane domain, a tyrosine kinase domain, and a carboxyl terminal. (Trenker and Jura, Curr Opin Cell Biol. 2020, 63: 174-185.) Under a normal physiological state, inactivated receptor tyrosine kinases exist in a monomer form. When its extracellular domain binds to signaling molecules, a receptor molecule monomer dimerizes or oligomers on a cell membrane, tyrosine residues in the intracellular domain are phosphorylated, functions of the kinases are activated, and a signaling complex is formed at ends of the receptor tyrosine kinases. The phosphorylated tyrosine site of the intracellular domain becomes a binding site for downstream signal proteins in the cells. The bound downstream signal protein is activated, expands information by using different signal transduction pathways, and causes a series of biochemical reactions in the cells, or causes a comprehensive response of the cells by using multiple pathways of an information matrix, and then controls processes such as cell proliferation, migration, differentiation and apoptosis (Lemmon M A, Schlessinger J. Cell. 2010; 141:1117-34).

Dysregulation of RTK signals may lead to various abnormal states in the cells, and RTK dysregulation plays an important role in development and regulation of cancer-like cells. RTK mutations may activate a series of subordinate associated reactions, disrupt balance between cell growth/proliferation and cell death, and trigger tumor development driven by dysregulation of RTKs. In human cancer, main mechanisms leading to dysregulation of constitutive RTK activation include functional gain mutations, genome amplification, chromosome rearrangement, autocrine activation, and kinase domain duplication. Many experiments prove that the tyrosine kinase family is closely related to pathogenesis of tumors, and inhibiting the receptor tyrosine kinases is an effective measure for treating cancer. Therefore, the RTKs have become an important target for developing therapeutic means for the tumors. A wide variety of RTK inhibitors based on a small molecule compound or a monoclonal antibody have been developed as drugs for treating various tumors (Punit S. Oncogene 2021, 40(24):4079-4093). Compared with traditional chemotherapeutics, the RTK inhibitor (TKI) has the advantages of high selectivity, few side effects and the like, but such a signal transduction inhibitor can only block a part of signal pathways of tumor cells, and compensation mechanisms of other signal pathways often weaken the therapeutic effect. In addition, the application of a targeted drug TKI may cause the tumors to have drug resistance, greatly reducing the therapeutic effect. Molecular mechanisms for resistance to the tyrosine kinase inhibitors include kinase overexpression and mutation, drug uptake, drug binding, drug efflux mediated by adenosine triphosphate binding transporter, deficiency of DNA repair mechanisms, activation of aberrant signaling pathways, epigenetic modifications, and tumor microenvironment, and the like. Therefore, it is urgent to develop new methods and technological means for various diseases caused by dysregulation of the RTKs.

Discoidin domain receptors (DDRs), as a member of the receptor tyrosine kinases (RTKs), play an important role in controlling signal transduction pathways for cell proliferation and differentiation. The dysregulation of DDRs is closely related to various diseases, including cancer, neurological degeneration, chronic inflammation, and fibrosis. The DDRs can promote malignant proliferation of tumor cells and are associated with invasion and metastasis of the tumor cells. An in-depth study of DDR regulation means can open up a new pathway for prevention and treatment of diseases such as the tumors in clinical practice. According to research, members of the DDR family, including a DDR1 and a DDR2, are widely expressed in various tissues. The DDR1 is mainly expressed in epithelial cells, smooth muscle cells, fibroblasts, oligodendrocytes, and macrophages in lungs, kidneys, colon, and brain, while the DDR2 is mainly expressed in fibroblasts, myofibroblasts, smooth muscle cells, and chondrocytes in kidneys, skin, lungs, heart, and connective tissue.

The DDRs are composed of three main structural domains, namely an extracellular ligand binding region, a transmembrane region, and an intracellular tyrosine kinase-containing region. The extracellular domain is composed of a discoidin domain and a discoidin-like domain capable of binding to ligand collagen; the transmembrane domain includes an extracellular juxtamembrane domain and a transmembrane helical region, and the region may mediate collagen-independent receptor dimerization (Yeung D A, J mol Biol. 2019; 431:368-390); and the intracellular domain includes an intracellular juxtamembrane domain and a catalytic tyrosine kinase domain. In addition, the DDRs have activation mechanisms different from other RTKs, which require collagen binding stimulation to initiate downstream pathways, resulting in autophosphorylation. A process of activating the DDRs has collagen specificity, that is, the DDR1 and the DDR2 require different types of collagen activation. The DDR1 preferentially binds to collagen I-V and VIII collagen, but has a lower affinity for X-type collagen. Research has shown that the function of DDR1 is mainly achieved through three pathways, namely, a classic collagen DDR1 signaling pathway, which relies on DDR1 kinase activity and directly affects downstream molecules such as Shc, Nck2, and Shp-2; or, a non-classical DDR1 signaling pathway that rely on collagen binding but do not rely on DDR1 kinase activity; or, it can function without relying on collagen stimulation or DDR1 kinase activity

Studies found that DDR1 is expressed in lung cancer, breast cancer, brain tumor, ovarian cancer, esophageal cancer, head and neck tumor, liver cancer, testicular cancer and other tumors; and the high expression is closely related to poor tumor prognosis (Rammal H, Front Pharmacol. 2016; 7:55). For example, the expression of DDR1 in cancer cell tissues of non-small cell lung cancer patients is positively correlated with mortality rate. The expression and phosphorylation levels of DDR1 are significantly increased in lung cancer tissues. Immunohistochemical analysis of 171 cases of non-small cell lung cancer showed that the positivity rate of DDR1 in aggressive non-small cell lung cancer is as high as 61% (Yang S H. Oncol Rep. 2010; 24:311-319). Meanwhile, DDR1 promote the proliferation and growth of various malignant tumor cells. For example, in human colon cancer and breast cancer, DDR1 can upregulate the anti apoptotic protein Bcl xL by activating downstream signaling proteins Ras/Raf/ERK and PI3K/Akt pathways, thereby enabling cancer cells to survive under toxic stress (Matadag S P. Medical Chemistry Research, 2021, 30 (3): 535-551).

Activated by natural type IV collagen, DDR1 can increase the expression of CD9 in breast cancer MDA-MB-231 cell line, leading to the migration of the cancer cells. DDR1 also induces the secretion and invasion of matrix metalloproteinase-2 and metalloproteinase-9 through DDR1 and Src dependent pathways, increasing their cellular expression levels. DDR1 activation plays an important role in the invasive ability and metabolic activity of breast cancer cells. Similarly, the upregulation of these two metalloproteinases caused by DDR1 is also a necessary condition for the metastasis and invasion of liver cancer cells (Lee, JH. Sci. Rep. 2018, 8, 1). In addition, in vitro and in vivo experiments have shown that DDRs are involved in the differentiation of tumor cells. Furthermore, they promote malignant cell transformation and tumor invasion and metastasis by disrupting normal signal transduction in the extracellular matrix, which prevent T cell infiltration by helping tumor cells establish a barrier around them. It has been shown that DDR1 can increase the invasion and migration ability of non-small cell lung cancer cells by promoting epithelial mesenchymal cell transformation. DDR1 can also regulate T cells CD4+ and CD8+inhibit the anti-tumor immune response, thus promoting the growth of breast cancer (Zhong, X. Oncol Rep. 2019; 42 (6): 2844-2854); DDR1 can weaken the immune system's clearance of cancer cells by affecting the tumor microenvironment. DDR1 can present a highly ordered extracellular matrix (ECM) during tumor development, affecting the infiltration of immune cells and weakening their ability to kill tumor cells. Other studies have shown that in the knockout of DDR1 mouse model, the infiltration of T cells within the tumor is improved and tumor growth is inhibited. It can be inferred that, Knockout or inhibition of DDR1 protein by DDR1 gene is a potential method to block the anti immune monitoring ability of tumors. DDR1 may become a new target for tumor immunotherapy (Sun X, Nature, 2021599 (7886): 673-678). Preclinical studies suggest that DDR1 inhibitors possess a wide range of anti-tumor activities in tumor transplantation mouse models derived from patients with non-small cell lung cancer, gastric cancer, esophageal cancer, liver cancer, breast cancer and colorectal cancer.

In addition to its role in the pathogenesis of tumors, DDRs are also key mediators for the secretion of a variety of inflammatory cytokines. They are dysfunctional in a variety of inflammatory diseases, including osteoarthritis, organ fibrosis and atherosclerosis (Leitinger, B. Int. Rev. Cellmol. Biol. 2014310, 39-87). DDR1 is also associated with fibrosis of liver, kidney, lung tissue, and blood vessels. Study has confirmed that DDR1 not only directly stimulates the secretion of inflammatory factors, but also enhances the effects of other pro-inflammatory cytokines and bacterial products (Matsuyama, W. J. Immunol. 2004, 172, 2332-2340). In various kidney disease models, inhibition of DDR expression can prevent inflammatory changes and fibrosis in the kidneys. Experimental results have shown that DDR1 deficiency can effectively reduce bleomycin induced pulmonary inflammation and fibrosis (Avivi-Green, C. Am. J. Respir. Crit. Care Med. 2006, 174, 420-427). In a model of anti glomerular basement membrane glomerulonephritis with prominent inflammation and unilateral ureteral obstruction, it was found that inhibiting DDR1 expression can reduce the migration of inflammatory cells and hinder fibrosis formation (Guerrot, D Am. J. Pathol. 2011, 179, 83).

As a unique member of the RTKs family, DDRs, especially DDR1, are closely related to the occurrence and development of cancer, inflammation, fibrosis, and neurodegenerative diseases, and are considered potential important targets for intervention and treatment of these diseases. The regulation and inhibition of RTKs are usually achieved through small molecule inhibitors that antagonize intracellular kinase activity or antibody drugs that interfere with extracellular domain function. Similarly, DDR1 inhibitors can also be divided into monoclonal antibodies that act on the extracellular binding region and small molecule inhibitors that act on the intracellular kinase domain and affect downstream signaling pathways. The latter can be further divided into multi-target RTK inhibitors and selective DDR1 inhibitors. Because the structure of the kinase ATP binding region of some members of the RTKs family has high homology, especially DDRs and c-Kit, many RTK multi target kinase inhibitors exhibit certain DDR1 inhibitory activity between Bcr-Abl kinases. For example, multi-target inhibitors Imatinib, Dasatinib, and Nilotinib can inhibit collagen induced self phosphorylation of DDR1. At present, various marketed Bcr Abl inhibitors, such as Ponatinib, Bosutinib, and Bafitinib, exhibit strong DDR1 inhibitory activity. In addition, P38 MAPK inhibitor Doramapimod, the B-Raf/VGFR dual target inhibitor Sorafenib and the c-Met/VEGFR-2 dual target inhibitor Foretinib kinase inhibitor all have strong binding affinity to the DDR1 kinase domain. In recent years, a considerable number of highly selective DDR1 inhibitors based on the rational design of DDR and small molecule three-dimensional molecular structures have been discovered and developed, many of which have potential applications in tumors, neurodegenerative diseases, Alzheimer's disease, dry eye syndrome, inflammatory and fibrotic diseases (William A D Biomolecules. 2021, 11 (11): 1671). DDR1 small molecule inhibitors include different types of core structures such as urea, quinoline, isoquinoline, tetrahydroisoquinoline, 1H-indole, spirocycloindoline, and triheterospirocyclodecanone (WO2017/038870, WO2016064970A1, WO2015060373A1, CN108276388A, WO2017137334A1, WO2017/038871).

Although the biological activity of these DDR inhibitors has been confirmed by many experiments, like most other RTK inhibitors, these small molecule drugs rely on site occupying competition for ATP binding domains. Long-term use of them can cause cancer cell resistance. Meanwhile, RTK has both kinase catalytic function and non catalytic function in other domains. Relying solely on small molecule inhibitors for site competition cannot offset the full function of RTK. Therefore, it is urgent to find more effective means to regulate DDR1 and other RTK functions.

The use of the ubiquitin protease system (UPS) in eukaryotic cells to regulate intracellular protein levels has received great attention in the past decade. UPS can efficiently and specifically induce protein degradation for negative signal function regulation, and can degrade 80%-90% of ubiquitinated proteins in cells. It plays an extremely important role in maintaining the levels of various proteins in cells, involving almost all life activities such as regulating cell cycle, proliferation, apoptosis, metastasis, gene expression, signal transduction, etc. The ubiquitin protein, composed of 76 amino acids, is a highly conserved protein commonly found in eukaryotic cells. The ubiquitination labeling of proteins initiates the degradation of substrate proteins, which is carried out through the synergistic action of Ubiquitin activating enzyme E1, Ubiquitin binding enzyme E2, and E3 ubiquitin ligase. Firstly, ubiquitin forms a high-energy thioester bond with the carboxyl group on its C-terminal glycine and the essential cysteine thiol group on the ubiquitin activating enzyme E1, connecting to E1 and becoming an activated state of ubiquitin; Secondly, activated ubiquitin is transferred from ubiquitin activating enzyme E1 to ubiquitin binding enzyme E2; Finally, under the action of E3 ubiquitin ligase, the ubiquitin molecules connected to ubiquitin binding enzyme E2 are covalently linked to the substrate protein through isomeric peptide bonds. After ubiquitination, the substrate protein can be transported to the 26S proteasome or enter lysosomes for digestion and degradation. The specific recognition ability of E3 ubiquitin ligase towards substrate proteins determines the specificity of ubiquitin mediated protein degradation.

The Proteolytic Targeting Chimera (PROTAC) technology utilizes the intracellular ubiquitin proteasome system to degrade specific proteins. This technology will be able to combine small molecule ligands targeting specific proteins to form a bifunctional compound by connecting them with ligands of E3 ubiquitin ligase through bridge chain fragments. By optimizing the link position and bridge chain length, the ligands at both ends of the PROTAC molecule are simultaneously bound to the target protein and E3 ubiquitin ligase, forming a target protein PROTACs E3 ligase ternary complex, which promotes ubiquitination labeling of the target protein and is subsequently degraded by the protease system. Due to the characteristics of the formation of ternary complexes, the target protein ligands used in PROTAC technology do not need to have strong target binding activity, so they can target proteins that traditional inhibitors cannot be used as drugs, such as skeleton proteins and transcription factors. In addition, the overall degradation of target proteins helps to overcome the problem of resistance to small molecule inhibitors. PROTAC molecules undergo repeated cyclic reactions through a catalytic mechanism. The degradation induced by PROTAC is event driven, not site occupying driven. After forming a complex and completing ubiquitin transfer, the drug dissociates and transfers to the next target in an enzymatic manner. To a certain extent, it is possible to maintain effective drug activity at low doses. The technical difficulties of PROTAC lie in the relationship between target protein degradation and generation rate, the ability of target protein to bind ubiquitin, the conformations and sites of target protein ligands and E3 ubiquitin ligase ligands, the length and composition of bridge chains, and the concentration that affect the formation and stability of ternary complexes. Therefore, regulation is more challenging. Among the over 600 known E3 ubiquitin ligases, only a limited number are applied to the design of PROTAC compounds, including CRBN, VHL, MDM2 and CIAP1 classes. These E3 ubiquitin ligases endow substrates with specificity to achieve target protein ubiquitination. The Von Hippel Lindau (VHL) tumor suppressor factor in common E3 ubiquitin ligases is composed of extension B and C Cul2 and Rbx1 are composed, and their main substrate is hypoxia inducible factor 1 (HIF-1). The ligand study of E3 ubiquitin ligase VHL revealed the crystal structure of its complex, confirming that small molecule compounds can mimic the main substrate of transcription factor HIF-1 (CN108601764A). During the application process, The binding affinity between VHL ligands and E3 ubiquitin ligase is relatively weak, which can easily lead to incomplete degradation of target proteins and cause off target effects. Another important E3 ligase, Cereblon (CRBN), is a protein encoded by the human CRBN gene. The CRBN homologous genes are highly conserved, demonstrating their importance in physiology. Cereblon and damaged DNA binding protein 1 (DDB1) Cullin-4A (CUL4A) and Cullin-1 regulator (ROC1) form the E3 ubiquitin ligase complex. This complex can ubiquitinate a series of proteins. Considering the binding ability of the phthalimide structural unit to CRBN, this structural unit is often used as an E3 ligase recruiting ligand to hijack CRBN for the degradation of target proteins. So far, there are many experimental drugs developed using PROTAC technology, including nuclear protein receptor degrading agents, kinase degrading agents, transcription factor degrading agents, etc. PROTACs targeting RTK can block kinase matrix signaling, preventing inactivated kinases from transmitting oncogenic gene signals through their skeletal functions, leading to sustained loss of RTK function. The existing RTK degradation agents can degrade kinases including wild-type or mutant EGFR HER2, c-MET, etc. The present invention designs and develops degradation agents targeting tyrosine kinase receptors with unique structures, particularly PROTAC degradation agents targeting DDR1. These compounds can treat related diseases by degrading abnormal tyrosine kinase receptors, thus having significant clinical significance.

### SUMMARY OF THE INVENTION

The present invention is firstly to solve the technical problems of providing a bifunctional compound for degrading receptor tyrosine kinases or a pharmaceutically acceptable salt, stereoisomer, solvate, or polymorphic form thereof, wherein such compounds degrade specific receptor tyrosine kinases, particularly discoidin domain receptors (DDRs), regulating downstream key proteins and further playing a therapeutic role in related diseases, and demonstrating outstanding ability to inhibit tumor cell proliferation;

The present invention is secondly to solve the technical problems of providing a preparation method and application of a bifunctional compound for degrading receptor tyrosine kinase.
the X part is a ligand capable of binding to receptor tyrosine kinase; preferably, the X part is a ligand capable of binding to discoidin domain receptors (DDRs); more preferably, the X part is a ligand capable of binding to a DDR1 in a discoidin domain receptor family;
the Y part is a ligand capable of binding to E3 ubiquitin ligase; preferably, the Y part is a ligand capable of binding to E3 ligase Cereblon; more preferably, the Y part is a ligand capable of binding to a Von Hippel-Lindau tumor inhibiting factor; and
the L part is a connecting group that connects the X part with the Y part.

Specifically, the bifunctional compound of claim 1, wherein the X part is selected from following structures as shown in X1 to X6, wherein a zigzag line represents a position where the X part is connected with the L part through covalent bonds;

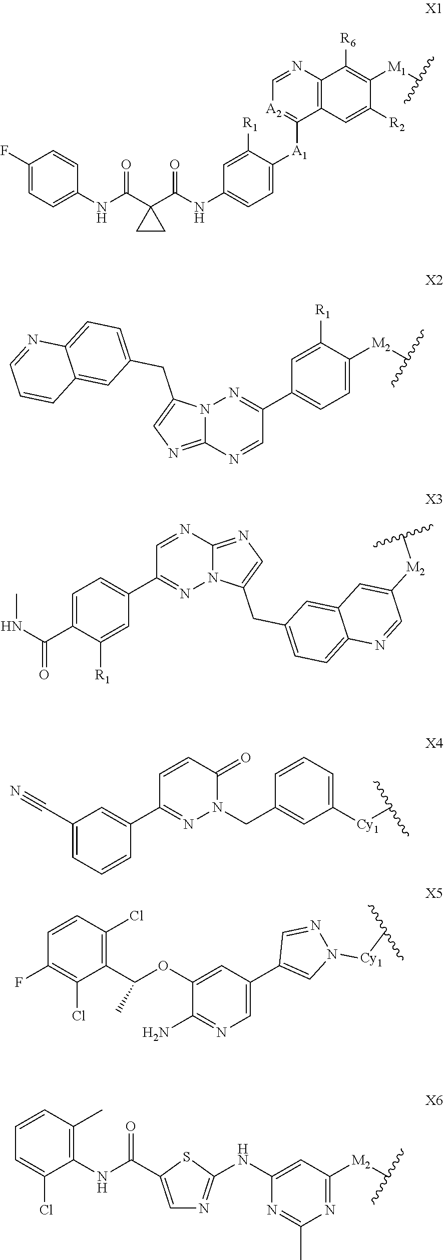

wherein,
the R₁ is selected from H, F, or Cl;
the R₂ is selected from H, —OR₃, —NHR₃, F, Cl, or Br;
preferably, in the —NHR₃, the R₃ is selected from H, a C1-4 linear or branched alkyl, or a C1-4 linear or branched alkyl with any hydrogen atom being substituted by fluorine, or ArCO—, wherein, the Ar is selected from H, halogen, a C1-4 linear or branched alkyl, a C1-4 linear or branched alkoxy, or a C1-4 linear or branched amino substituted 5-8-membered aromatic ring group;
preferably, the Ar is preferably H, halogen, a C1-4 linear or branched alkyl, a C1-4 linear or branched alkoxy, or a C1-4 linear or branched amino substituted phenyl;
the R₆ is selected from H, F, Cl, OR₇ or NHR₇;
preferably, in the NHR₇, the R₇ is selected from H or a C1-4 linear or branched alkyl;
the A₁ is selected from O or NH;
the A₂ is selected from N or CH;
the M₁ is selected from NR₃, CH₂, O, C or Cy₁;
the M₂ is selected from NH, O, CO or Cy₁;
the Cy₁ is selected from a substituted or unsubstituted 4-7-membered carbon heteromonocyclic ring, a substituted or unsubstituted 5-10-membered carbon heterofused ring, a substituted or unsubstituted 4-7-membered monocyclic alkyl, a substituted or unsubstituted 5-10-membered cycloalkyl, a substituted or unsubstituted 7-10-membered bridged cycloalkyl, or a substituted or unsubstituted 5-8-membered aromatic ring group;
preferably, in Cy₁, the substitution comprises substitution optionally by 0 to 3 substituents selected from H, F, Cl, OH, COOH, CN, NH₂, a carbonyl, a C1-4 linear or branched alkyl, a halogen-substituted C1-4 linear or branched alkyl, a hydroxyl-substituted C1-4 linear or branched alkyl or a C1-4 alkoxy;
preferably, in the Cy₁, a heteromonocyclic ring and a heterofused ring contain 0 to 4 heteroatoms selected from O, S and N; and the aromatic ring group contains 0 to 3 heteroatoms selected from N, S and O;
more preferably, the Cy₁ is selected from pyridine, piperazine, pyrimidine, pyrazine, pyridazine, benzene, pyrazole, imidazole, triazole, 2,2-difluoropiperidine, 2,2-difluoropiperazine, 2,2,3,3-tetrafluoropiperazine, 2,2,5,5-tetrafluoropiperazine or 2,2,6,6-tetrafluoropiperazine.

Specifically, the bifunctional compound of claim 1 or 2, wherein the Y part is a VHL ligand, and the Y part is selected from following structures as shown in Y1 to Y3, wherein an asterisk (x) position represents a position where the Y part is connected with the L part through covalent bonds;

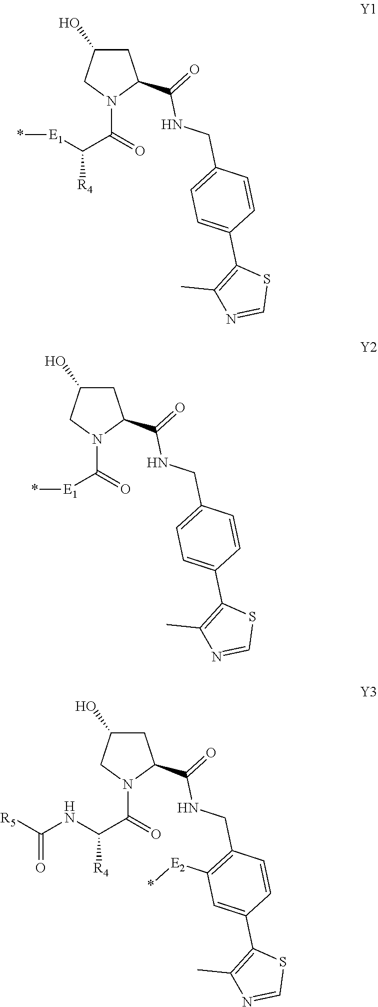

wherein,
the R₄ is selected from H, a C1-C5 linear or branched alkyl, a C3-C6 cycloalkyl, and a 3-6 membered heterocycloalkyl;
preferably, the heterocycloalkyl contains 1 to 3 heteroatoms selected from O, N, and S;
preferably, the R₄ is selected from an isopropyl, a tert butyl, a cyclohexyl or a tetrahydropyranyl;
the R₅ is selected from a C1-C5 linear or branched alkyl, and a C3-C6 substituted cycloalkyl;
preferably, the R₅ is selected from a methyl, an ethyl, an isopropyl, or a 1-fluorocyclopropanyl;
the E₁ is selected from NH, NR₃, CH₂ or Cy₂;
preferably, the Cy₂ is selected from a substituted or unsubstituted 4-7-membered carbon heteromonocyclic ring or a substituted or unsubstituted 5-8-membered aromatic ring group;
preferably, the aromatic ring group and the carbon heteromonocyclic ring are substituted by 0 to 3 substituents selected from H, F, Cl, OH, COOH, CN, NH₂, a carbonyl, a C1-4 linear or branched alkyl, a halogen-substituted C1-4 linear or branched alkyl, a hydroxyl-substituted C1-4 linear or branched alkyl or a C1-4 alkoxy;
preferably, the heteromonocyclic ring contains 0 to 4 heteroatoms selected from O, S and N; and the aromatic ring group contains 0 to 3 heteroatoms selected from N, S and O;
preferably, the Cy₂ is selected from piperidine, piperazine, azetidine, or 1,2,3-triazole;
the E₂ is selected from O, NH or Cy₃;
preferably, the Cy₃ is selected from a 4-7-membered carbon heteromonocyclic ring;
preferably, the Cy₃ is selected from piperidine, piperazine, or azetidine

Specifically, the bifunctional compound of claim 1 or 2, wherein the Y part is a ligand of E3 ligase Cereblon, and the Y part is selected from a piperidine-2,6-diketone compound, thalidomide or its derivatives, lenalidomide or its derivatives, or pomalidomide or its derivatives.

Specifically, the bifunctional compound of claim 4, wherein the Y part has following structures as shown in Y4 to Y8, wherein an asterisk (x) position represents a position where the Y part is connected with the L part through covalent bonds;

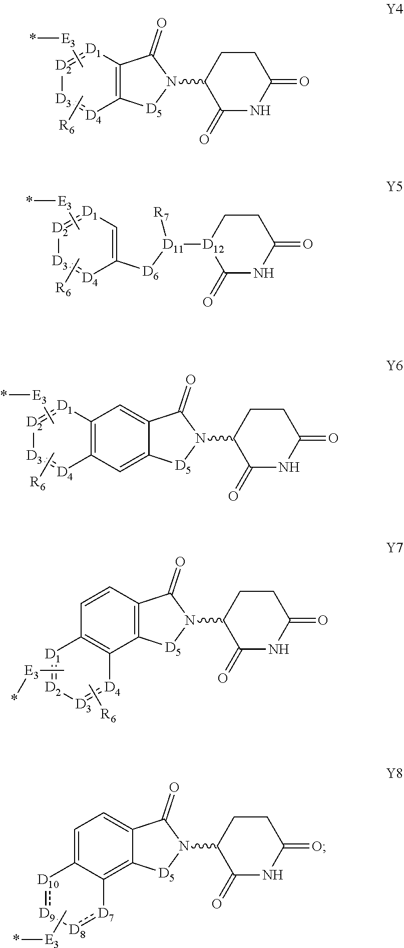

wherein,
D₁, D₂, D₃ and D₄ are selected from 0 to 2 N, CH, or CR₆ independently from each other;
preferably, the D₁, the D₂, the D₃ and the D₄ are independent of each other as CH or CR₆; or, when at least one of the D₁, the D₂, the D₃ or the D₄ is CH or CR₆, at least one of the remaining D₁, D₂, D₃ or D₄ is N;
the D₅ is selected from —(CH₂)_{q}—, —CF₂, CO, —W₁ [symbol] W₂— or —W₁ [symbol] W₂ [symbol] W₃—, wherein, q is optionally selected from natural numbers 0 to 5; the W₁, W₂ or W₃ are selected from CR₇R₈, N, NH, CO or CF₂ independently from each other, wherein, W₁ and W₂, as well as W₂ and W₃ are independently connected by covalent single bonds or double bonds respectively; and the R₇ or the R₈ are selected from H or a C1-4 linear or branched alkyl independently from each other;
the D₆ is selected from CH₂ or CO, or is a covalent bond;
the D₁ is selected from NR₇, or is a covalent bond;
the D₁₂ is selected from CH or N;
in the Y8, at least one of D₇, D₈, D₉ and D₁₀ is N or a covalent bond, while the remaining is selected from N, O, S, or CH₂ independently from each other;
the E3 and the R₆ are connected with D₁, D₂, D₃ or D₄ through covalent bonds independently from each other, and the D₁, the D₂, the D₃ or the D₄ at a connecting site is CH or CR₆C;
preferably, the E₃ is selected from NH, CH₂, CF₂, O, —CH═CH—, —C≡C—, —Cy₄ or Cy₄-Cy₅;
preferably, the R₆ is selected from H, F, Cl, OR₇ or NHR₇;
the Cy₄ is selected from a 4-7-membered carbon heteromonocyclic ring, a 5-10-membered carbon heterofused ring, a 4-7-membered monocyclic alkyl, a 5-10-membered cycloalkyl, a 7-10-membered bridged cycloalkyl, or a 5-8-membered aromatic ring group;
preferably, the aromatic ring group, the monocyclic alkyl, the carbon heteromonocyclic ring and the carbon heterofused ring are optionally substituted by 0 to 3 substituents selected from H, F, Cl, OH, COOH, CN, NH₂, a carbonyl, a C1-4 linear or branched alkyl, a halogen-substituted C1-4 linear or branched alkyl, a hydroxyl-substituted C1-4 linear or branched alkyl or a C1-4 alkoxy;
preferably, the heteromonocyclic ring and the heterofused ring contain 0 to 4 heteroatoms selected from O, S and N; and the aromatic ring group contains 0 to 3 heteroatoms selected from N, S and O;
preferably, the Cy₄ is selected from any substituted piperidine, piperazine, or azetidine;
the Cy₅ is selected from a 4-7-membered carbon heteromonocyclic ring, a 5-10-membered carbon heterofused ring, a 4-7-membered monocyclic alkyl, a 5-10-membered cycloalkyl, a 7-10-membered bridged cycloalkyl, or a 5-8-membered aromatic ring group;
preferably, the aromatic ring group, the monocyclic alkyl, the carbon heteromonocyclic ring and the carbon heterofused ring are optionally substituted by 0 to 3 substituents selected from H, F, Cl, OH, COOH, CN, NH₂, a carbonyl, a C1-4 linear or branched alkyl, a halogen-substituted C1-4 linear or branched alkyl, a hydroxyl-substituted C1-4 linear or branched alkyl or a C1-4 alkoxy;
preferably, the heteromonocyclic ring and the heterofused ring contain 0 to 4 heteroatoms selected from O, S and N; and the aromatic ring group contains 0 to 3 heteroatoms selected from N, S and O;
preferably, the Cy₅ is selected from any substituted azetidine, piperidine, or piperazine.

6. The bifunctional compound of any one of claims 1 to 5, wherein the L part is a covalent bond, and directly connects the X part with the Y part;
or,
the L part is selected from —O—, —C═O, —OCO—, —CH₂(CH₂)ₙCO—, —CH₂(CH₂)ₙ—, —CONHCH₂(CH₂)ₙCO—, —COCH₂(CH₂)ₙCO—, —O(CH₂)ₙ—, —O(CH₂)ₙCO—, —O(CH₂)ₙNH—, —NH(CH₂)ₙCO—, —CH₂CH₂N(R₃)CO—, —CH₂OCH₂CO—, or —CH₂Ar₁CO—, wherein the Ar₁ is selected from a benzene ring, a thiophene ring, or a 1H-pyrrole; and n is optionally natural numbers 0 to 14;
or,

The L part is selected from structures as shown below, wherein a zigzag line represents a position where the L part is connected with the X part through covalent bonds, and an asterisk (x) position represents a position where the L part is connected with the Y part through covalent bonds;

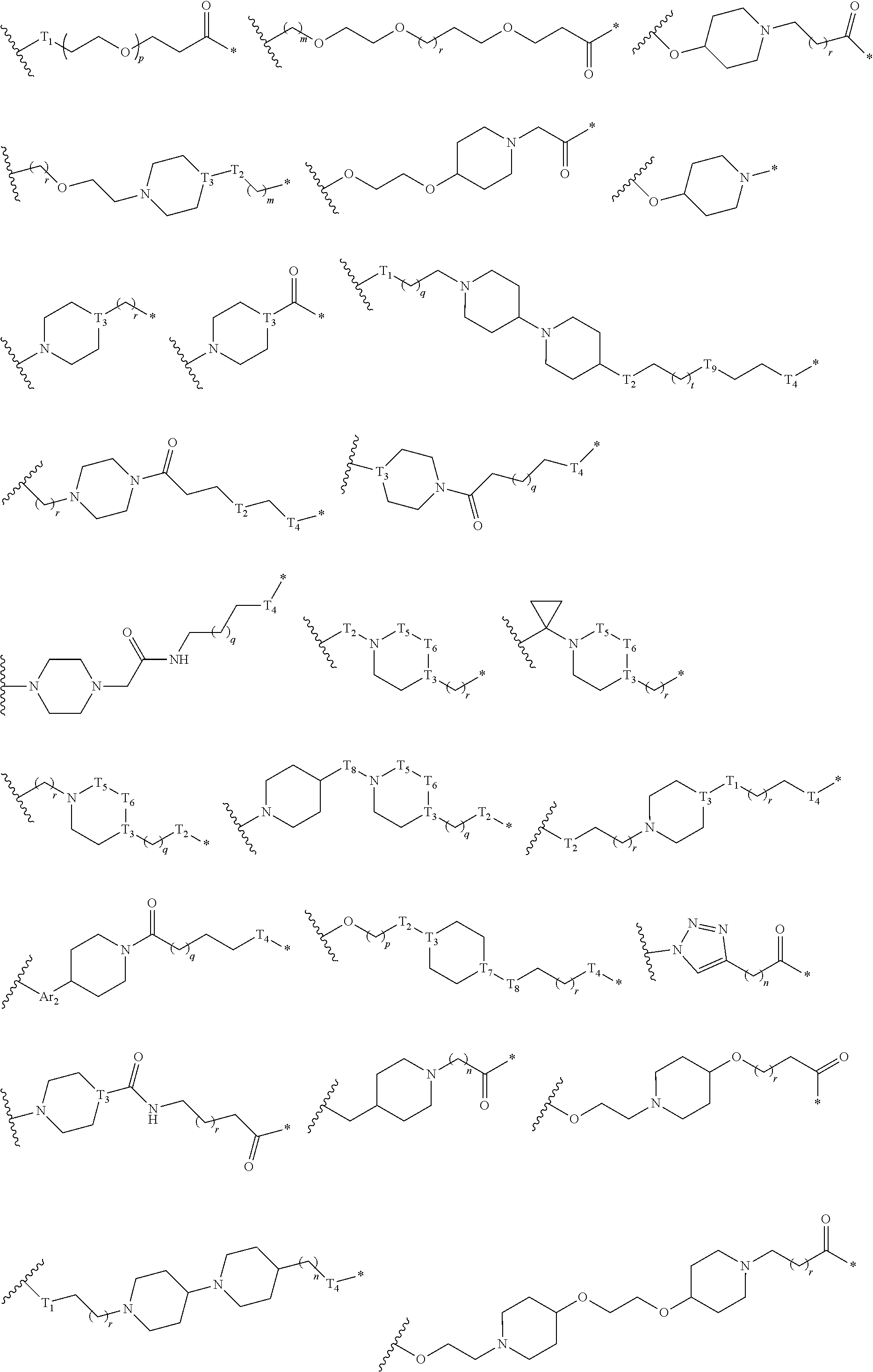

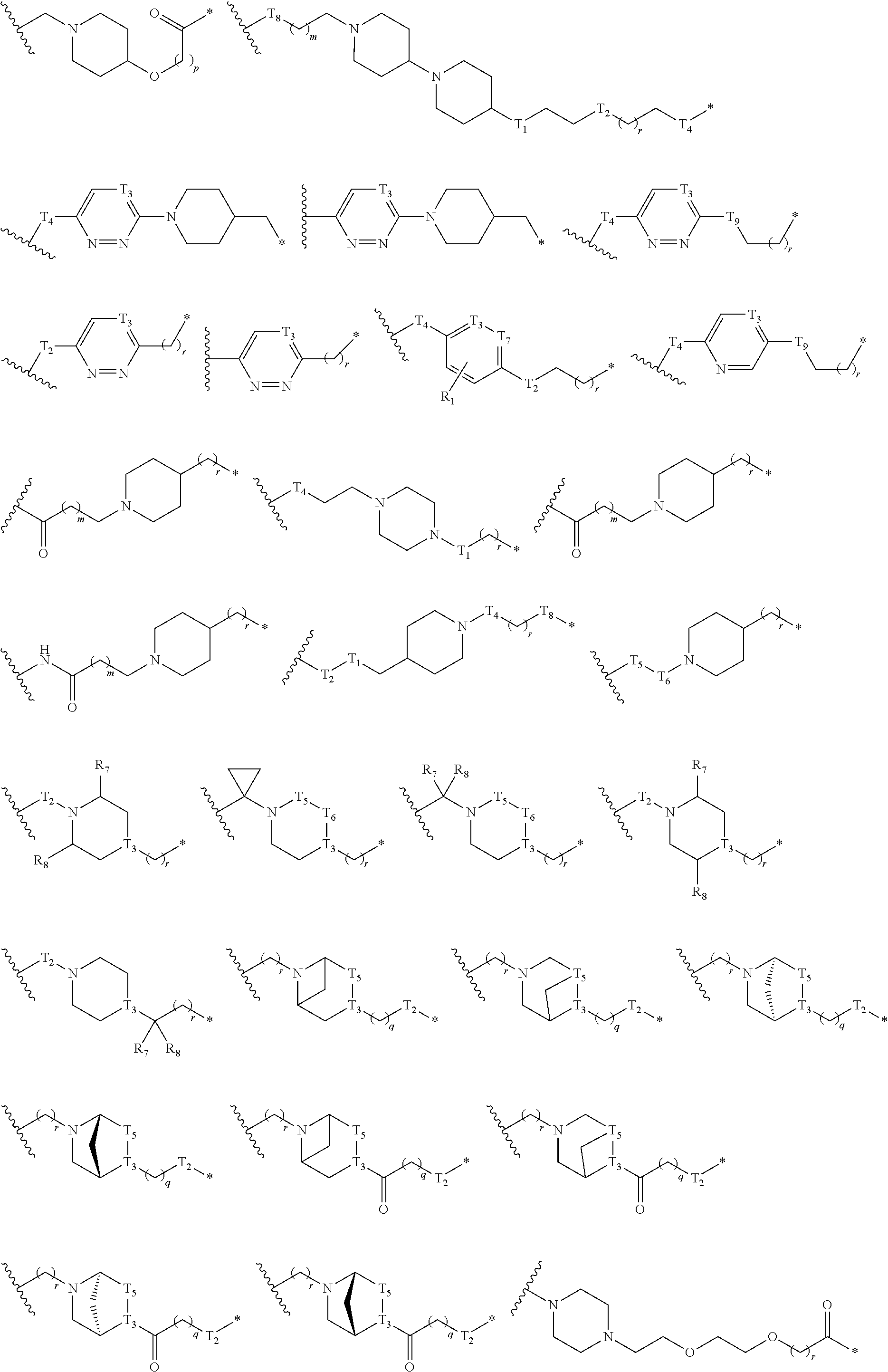

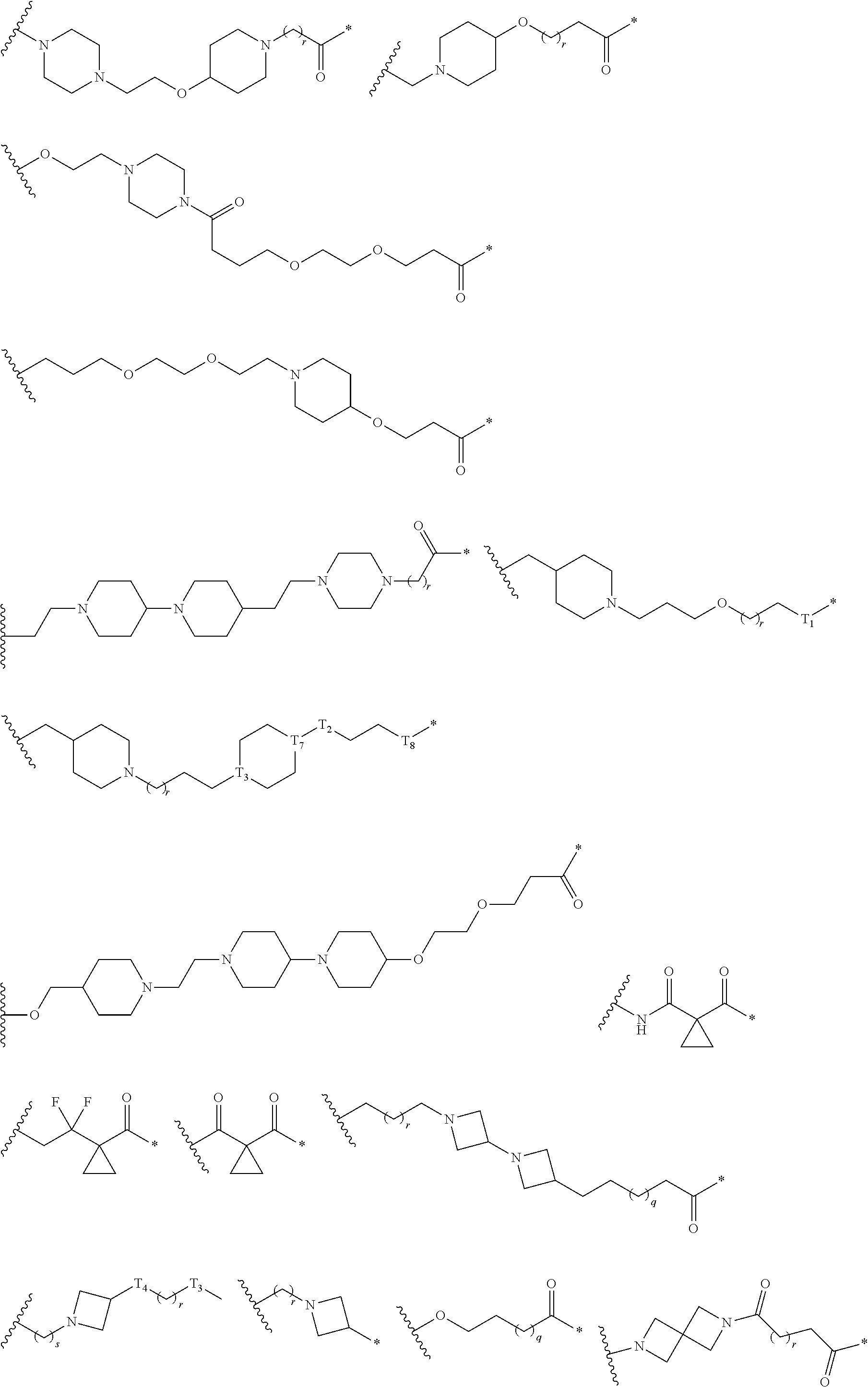

wherein,
the T₁ is selected from O, CH₂ or CO;
the T₂ is selected from O, CH₂, CF₂, NH or CO;
the T₃ and T₇ are selected from N or CH independently from each other;
the T₄ is selected from CH₂ or CO;
the T₅ and T₆ are selected from CH₂, CF₂ or CO independently from each other;
the T₈ is selected from O, CH₂, CF₂, NH or CO;
the T₉ is selected from O, NH or CH₂;
the Ar₂ is selected from a phenyl, an imidazole group or a pyrazolyl;
the R₈ is selected from H or a C1-4 linear or branched alkyl; and
m is optionally selected from natural numbers 0 to 2, n is optionally selected from natural numbers 0 to 12, p is optionally selected from natural numbers 1 to 6, q is optionally selected from natural numbers 0 to 5, and r is optionally selected from natural numbers 0 to 3;

Specifically, the bifunctional compound of any one of claims 1 to 6, wherein the compound is selected from any one of the following structures:

TABLE 1
compound Structure

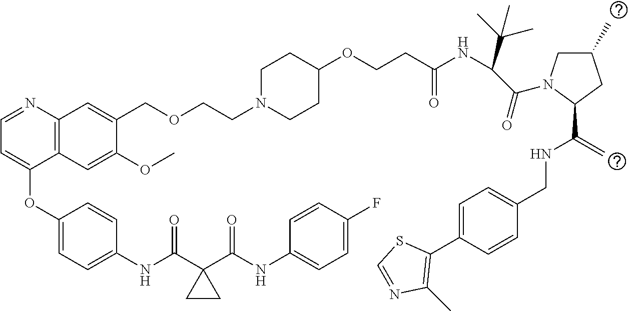 [SMILES: C=C(NCc1ccc(-c2scnc2C)cc1)[C@@H]1C[C@@H](C)CN1C(=O)[C@@H](NC(=O)CCOC1CCN(CCOCc2cc3nccc(Oc4ccc(NC(=O)C5(C(=O)Nc6ccc(F)cc6)CC5)cc4)c3cc2OC)CC1)C(C)(C)C]

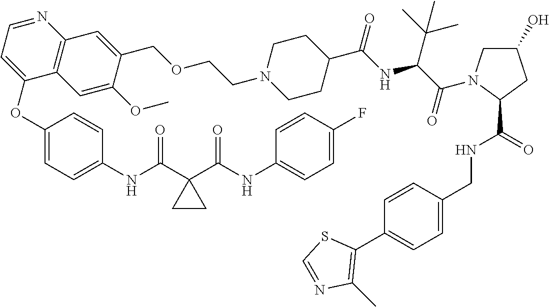 [SMILES: COc1cc2c(Oc3ccc(NC(=O)C4(C(=O)Nc5ccc(F)cc5)CC4)cc3)ccnc2cc1COCCN1CCC(C(=O)N[C@H](C(=O)N2C[C@H](O)C[C@H]2C(=O)NCc2ccc(-c3scnc3C)cc2)C(C)(C)C)CC1]

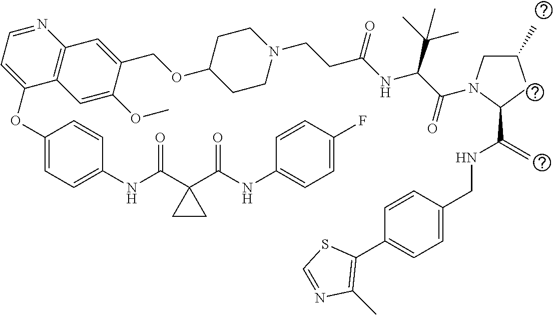 [SMILES: C=C(NCc1ccc(-c2scnc2C)cc1)[C@@H]1C[C@@H](C)CN1C(=O)[C@@H](NC(=O)CCN1CCC(OCc2cc3nccc(Oc4ccc(NC(=O)C5(C(=O)Nc6ccc(F)cc6)CC5)cc4)c3cc2OC)CC1)C(C)(C)C]

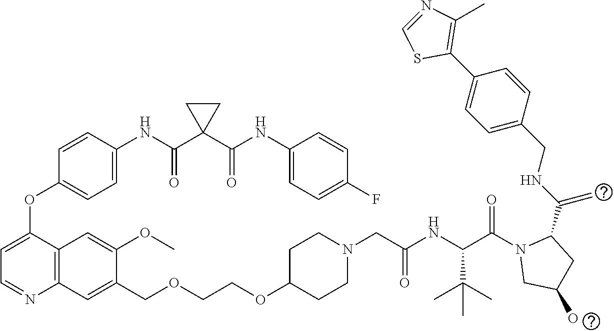 [SMILES: C=C(NCc1ccc(-c2scnc2C)cc1)[C@@H]1C[C@@H](O)CN1C(=O)[C@@H](NC(=O)CN1CCC(OCCOCc2cc3nccc(Oc4ccc(NC(=O)C5(C(=O)Nc6ccc(F)cc6)CC5)cc4)c3cc2OC)CC1)C(C)(C)C]

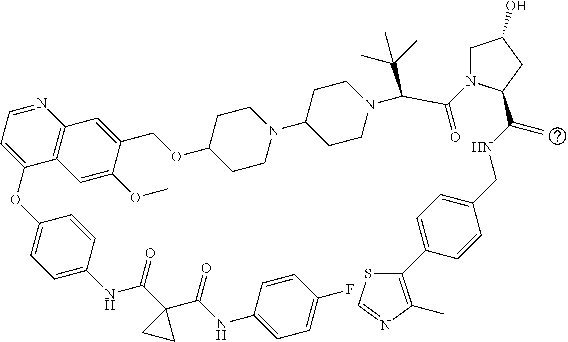 [SMILES: C=C(NCc1ccc(-c2scnc2C)cc1)[C@@H]1C[C@@H](O)CN1C(=O)[C@@H](N1CCC(N2CCC(OCc3cc4nccc(Oc5ccc(NC(=O)C6(C(=O)Nc7ccc(F)cc7)CC6)cc5)c4cc3OC)CC2)CC1)C(C)(C)C]

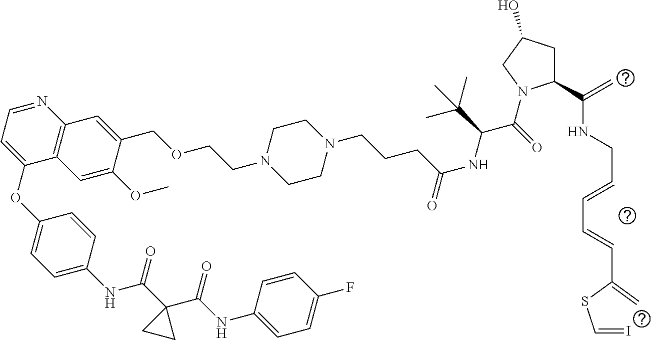

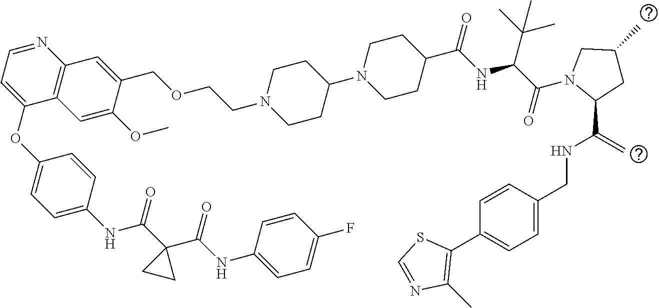 [SMILES: C=C(NCc1ccc(-c2scnc2C)cc1)[C@@H]1C[C@@H](C)CN1C(=O)[C@@H](NC(=O)C1CCN(C2CCN(CCOCc3cc4nccc(Oc5ccc(NC(=O)C6(C(=O)Nc7ccc(F)cc7)CC6)cc5)c4cc3OC)CC2)CC1)C(C)(C)C]

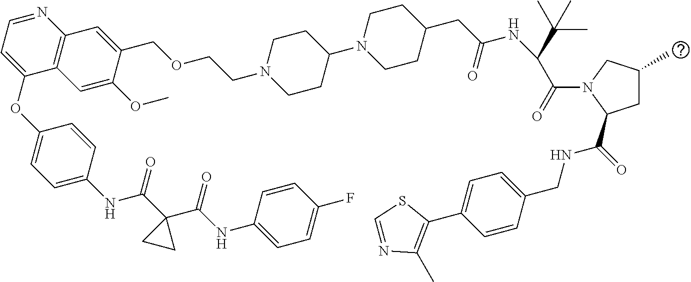 [SMILES: COc1cc2c(Oc3ccc(NC(=O)C4(C(=O)Nc5ccc(F)cc5)CC4)cc3)ccnc2cc1COCCN1CCC(N2CCC(CC(=O)N[C@H](C(=O)N3C[C@H](C)C[C@H]3C(=O)NCc3ccc(-c4scnc4C)cc3)C(C)(C)C)CC2)CC1]

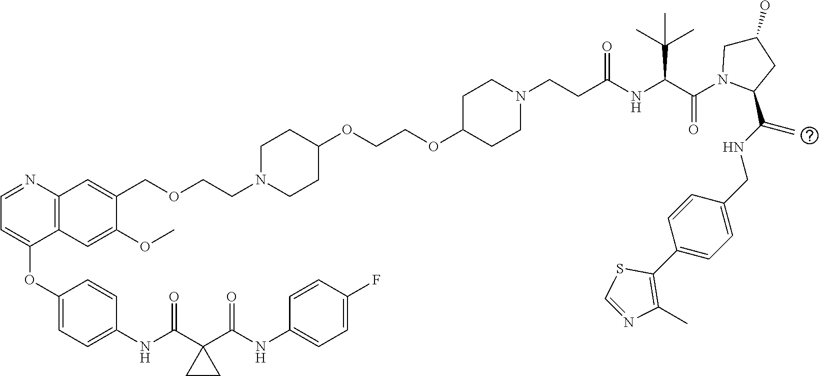 [SMILES: C=C(NCc1ccc(-c2scnc2C)cc1)[C@@H]1C[C@@H](O)CN1C(=O)[C@@H](NC(=O)CCN1CCC(OCCOC2CCN(CCOCc3cc4nccc(Oc5ccc(NC(=O)C6(C(=O)Nc7ccc(F)cc7)CC6)cc5)c4cc3OC)CC2)CC1)C(C)(C)C]

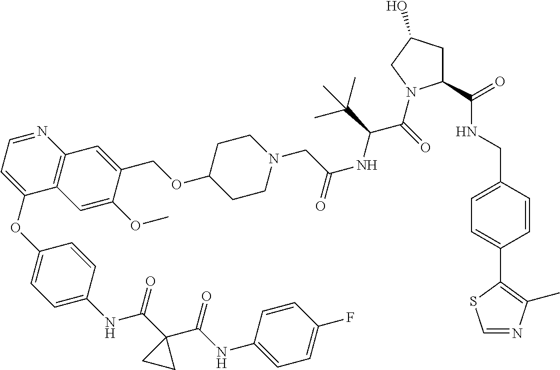 [SMILES: COc1cc2c(Oc3ccc(NC(=O)C4(C(=O)Nc5ccc(F)cc5)CC4)cc3)ccnc2cc1COC1CCN(CC(=O)N[C@H](C(=O)N2C[C@H](O)C[C@H]2C(=O)NCc2ccc(-c3scnc3C)cc2)C(C)(C)C)CC1]

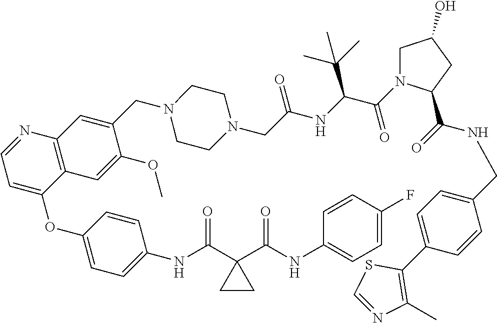 [SMILES: COc1cc2c(Oc3ccc(NC(=O)C4(C(=O)Nc5ccc(F)cc5)CC4)cc3)ccnc2cc1CN1CCN(CC(=O)N[C@H](C(=O)N2C[C@H](O)C[C@H]2C(=O)NCc2ccc(-c3scnc3C)cc2)C(C)(C)C)CC1]

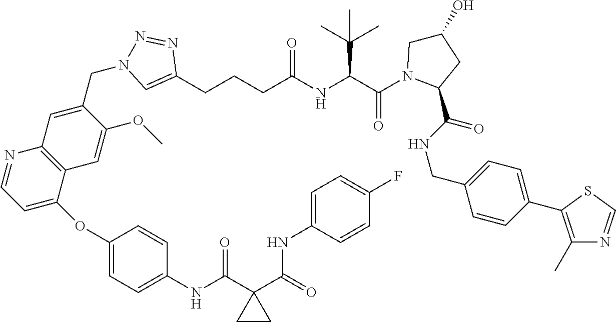 [SMILES: COc1cc2c(Oc3ccc(NC(=O)C4(C(=O)Nc5ccc(F)cc5)CC4)cc3)ccnc2cc1Cn1cc(CCCC(=O)N[C@H](C(=O)N2C[C@H](O)C[C@H]2C(=O)NCc2ccc(-c3scnc3C)cc2)C(C)(C)C)nn1]

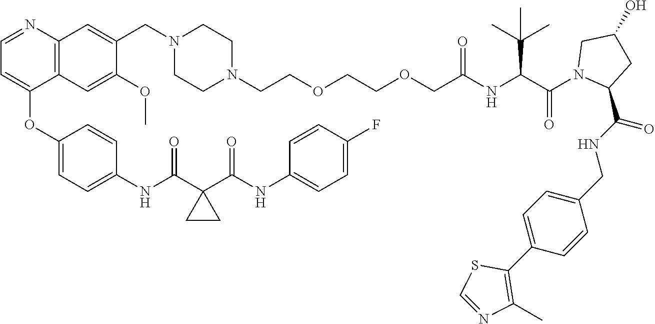 [SMILES: COc1cc2c(Oc3ccc(NC(=O)C4(C(=O)Nc5ccc(F)cc5)CC4)cc3)ccnc2cc1CN1CCN(CCOCCOCC(=O)N[C@H](C(=O)N2C[C@H](O)C[C@H]2C(=O)NCc2ccc(-c3scnc3C)cc2)C(C)(C)C)CC1]

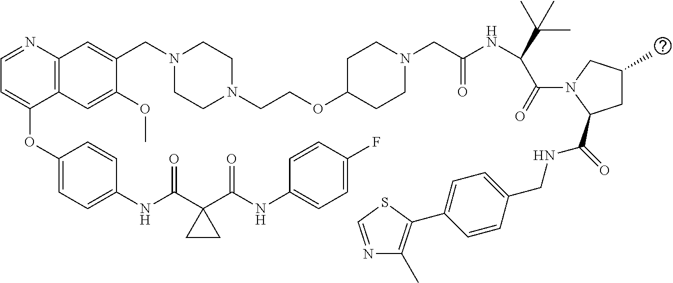 [SMILES: COc1cc2c(Oc3ccc(NC(=O)C4(C(=O)Nc5ccc(F)cc5)CC4)cc3)ccnc2cc1CN1CCN(CCOC2CCN(CC(=O)N[C@H](C(=O)N3C[C@H](C)C[C@H]3C(=O)NCc3ccc(-c4scnc4C)cc3)C(C)(C)C)CC2)CC1]

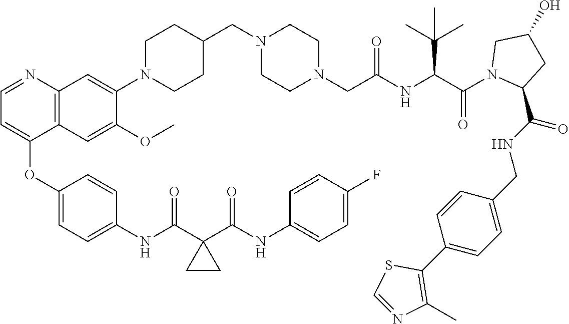 [SMILES: COc1cc2c(Oc3ccc(NC(=O)C4(C(=O)Nc5ccc(F)cc5)CC4)cc3)ccnc2cc1N1CCC(CN2CCN(CC(=O)N[C@H](C(=O)N3C[C@H](O)C[C@H]3C(=O)NCc3ccc(-c4scnc4C)cc3)C(C)(C)C)CC2)CC1]

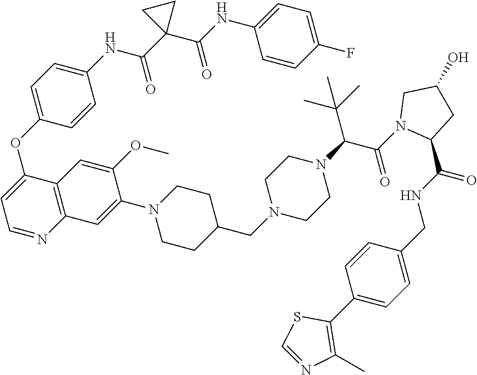 [SMILES: COc1cc2c(Oc3ccc(NC(=O)C4(C(=O)Nc5ccc(F)cc5)CC4)cc3)ccnc2cc1N1CCC(CN2CCN([C@H](C(=O)N3C[C@H](O)C[C@H]3C(=O)NCc3ccc(-c4scnc4C)cc3)C(C)(C)C)CC2)CC1]

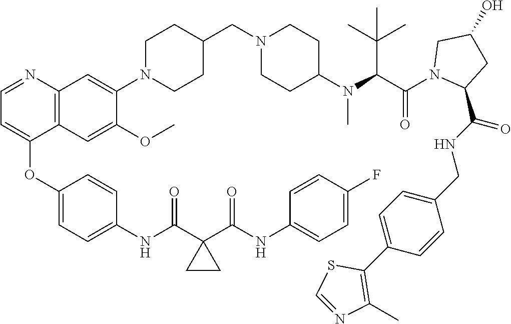 [SMILES: COc1cc2c(Oc3ccc(NC(=O)C4(C(=O)Nc5ccc(F)cc5)CC4)cc3)ccnc2cc1N1CCC(CN2CCC(N(C)[C@H](C(=O)N3C[C@H](O)C[C@H]3C(=O)NCc3ccc(-c4scnc4C)cc3)C(C)(C)C)CC2)CC1]

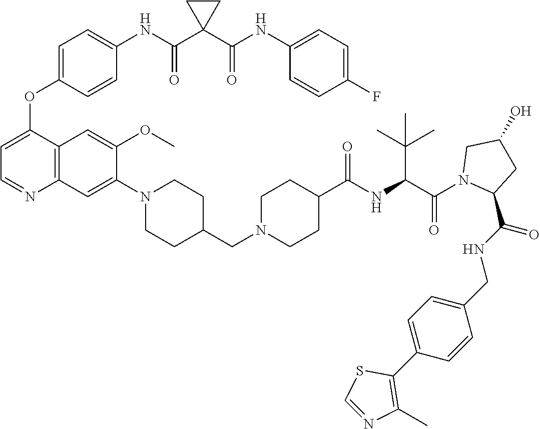 [SMILES: COc1cc2c(Oc3ccc(NC(=O)C4(C(=O)Nc5ccc(F)cc5)CC4)cc3)ccnc2cc1N1CCC(CN2CCC(C(=O)N[C@H](C(=O)N3C[C@H](O)C[C@H]3C(=O)NCc3ccc(-c4scnc4C)cc3)C(C)(C)C)CC2)CC1]

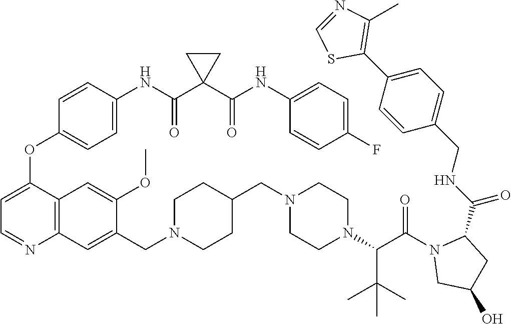 [SMILES: COc1cc2c(Oc3ccc(NC(=O)C4(C(=O)Nc5ccc(F)cc5)CC4)cc3)ccnc2cc1CN1CCC(CN2CCN([C@H](C(=O)N3C[C@H](O)C[C@H]3C(=O)NCc3ccc(-c4scnc4C)cc3)C(C)(C)C)CC2)CC1]

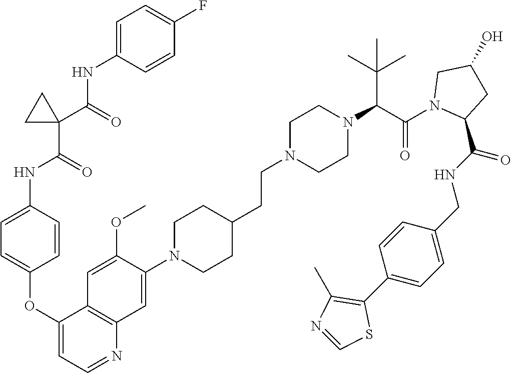 [SMILES: COc1cc2c(Oc3ccc(NC(=O)C4(C(=O)Nc5ccc(F)cc5)CC4)cc3)ccnc2cc1N1CCC(CCN2CCN([C@H](C(=O)N3C[C@H](O)C[C@H]3C(=O)NCc3ccc(-c4scnc4C)cc3)C(C)(C)C)CC2)CC1]

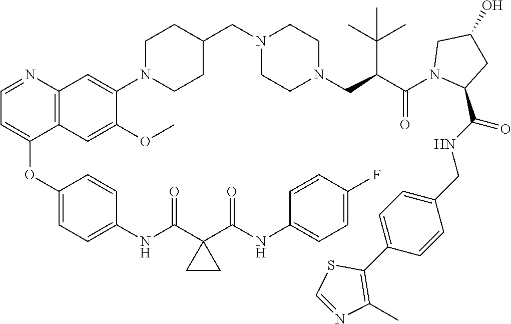 [SMILES: COc1cc2c(Oc3ccc(NC(=O)C4(C(=O)Nc5ccc(F)cc5)CC4)cc3)ccnc2cc1N1CCC(CN2CCN(C[C@H](C(=O)N3C[C@H](O)C[C@H]3C(=O)NCc3ccc(-c4scnc4C)cc3)C(C)(C)C)CC2)CC1]

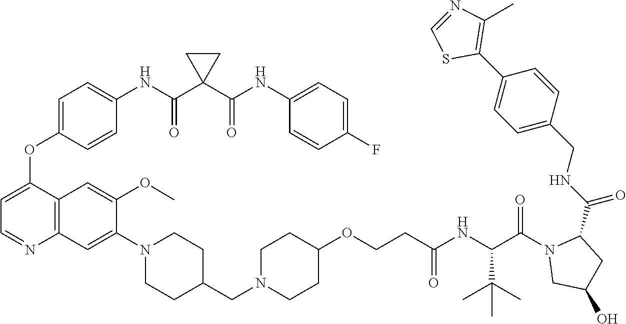 [SMILES: COc1cc2c(Oc3ccc(NC(=O)C4(C(=O)Nc5ccc(F)cc5)CC4)cc3)ccnc2cc1N1CCC(CN2CCC(OCCC(=O)N[C@H](C(=O)N3C[C@H](O)C[C@H]3C(=O)NCc3ccc(-c4scnc4C)cc3)C(C)(C)C)CC2)CC1]

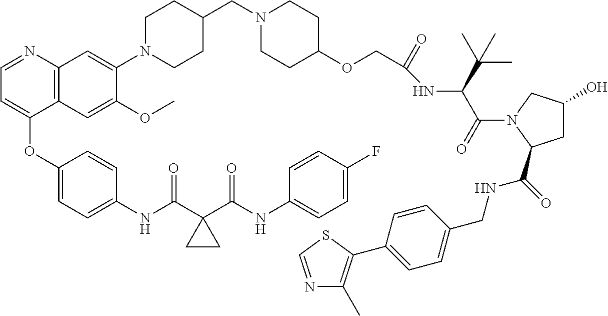 [SMILES: COc1cc2c(Oc3ccc(NC(=O)C4(C(=O)Nc5ccc(F)cc5)CC4)cc3)ccnc2cc1N1CCC(CN2CCC(OCC(=O)N[C@H](C(=O)N3C[C@H](O)C[C@H]3C(=O)NCc3ccc(-c4scnc4C)cc3)C(C)(C)C)CC2)CC1]

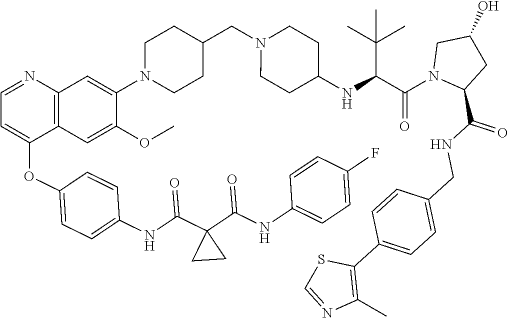 [SMILES: COc1cc2c(Oc3ccc(NC(=O)C4(C(=O)Nc5ccc(F)cc5)CC4)cc3)ccnc2cc1N1CCC(CN2CCC(N[C@H](C(=O)N3C[C@H](O)C[C@H]3C(=O)NCc3ccc(-c4scnc4C)cc3)C(C)(C)C)CC2)CC1]

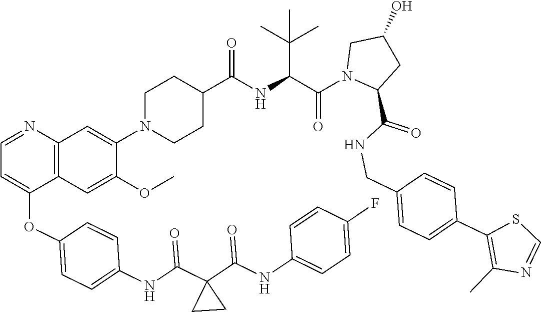 [SMILES: COc1cc2c(Oc3ccc(NC(=O)C4(C(=O)Nc5ccc(F)cc5)CC4)cc3)ccnc2cc1N1CCC(C(=O)N[C@H](C(=O)N2C[C@H](O)C[C@H]2C(=O)NCc2ccc(-c3scnc3C)cc2)C(C)(C)C)CC1]

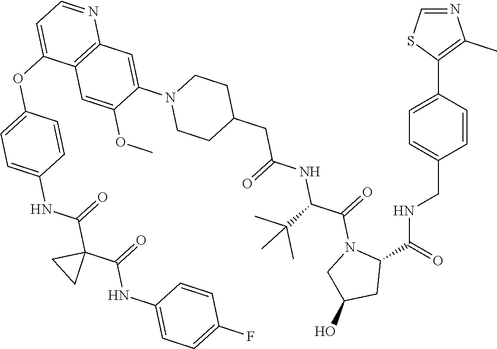 [SMILES: COc1cc2c(Oc3ccc(NC(=O)C4(C(=O)Nc5ccc(F)cc5)CC4)cc3)ccnc2cc1N1CCC(CC(=O)N[C@H](C(=O)N2C[C@H](O)C[C@H]2C(=O)NCc2ccc(-c3scnc3C)cc2)C(C)(C)C)CC1]

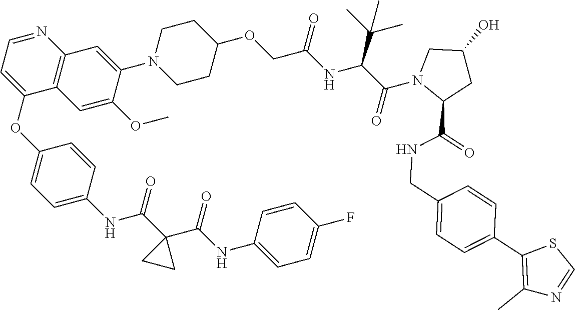 [SMILES: COc1cc2c(Oc3ccc(NC(=O)C4(C(=O)Nc5ccc(F)cc5)CC4)cc3)ccnc2cc1N1CCC(OCC(=O)N[C@H](C(=O)N2C[C@H](O)C[C@H]2C(=O)NCc2ccc(-c3scnc3C)cc2)C(C)(C)C)CC1]

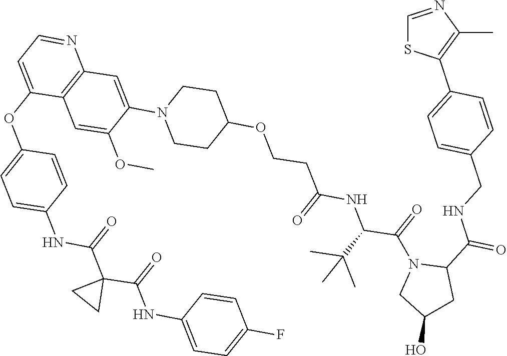 [SMILES: COc1cc2c(Oc3ccc(NC(=O)C4(C(=O)Nc5ccc(F)cc5)CC4)cc3)ccnc2cc1N1CCC(OCCC(=O)N[C@H](C(=O)N2C[C@H](O)CC2C(=O)NCc2ccc(-c3scnc3C)cc2)C(C)(C)C)CC1]

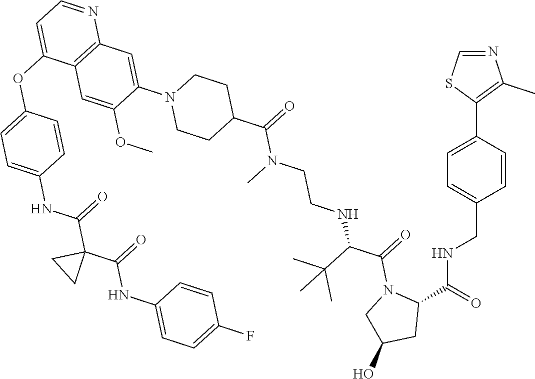 [SMILES: COc1cc2c(Oc3ccc(NC(=O)C4(C(=O)Nc5ccc(F)cc5)CC4)cc3)ccnc2cc1N1CCC(C(=O)N(C)CCN[C@H](C(=O)N2C[C@H](O)C[C@H]2C(=O)NCc2ccc(-c3scnc3C)cc2)C(C)(C)C)CC1]

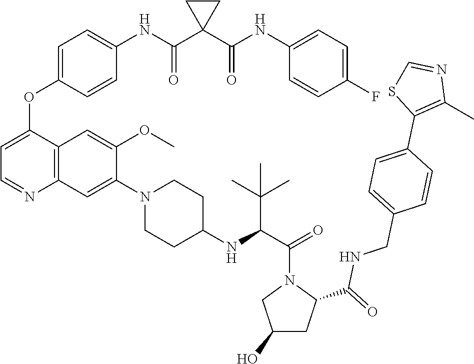 [SMILES: COc1cc2c(Oc3ccc(NC(=O)C4(C(=O)Nc5ccc(F)cc5)CC4)cc3)ccnc2cc1N1CCC(N[C@H](C(=O)N2C[C@H](O)C[C@H]2C(=O)NCc2ccc(-c3scnc3C)cc2)C(C)(C)C)CC1]

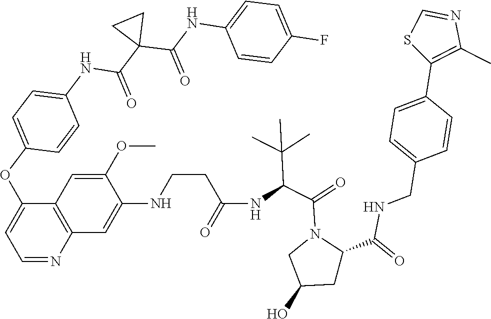 [SMILES: COc1cc2c(Oc3ccc(NC(=O)C4(C(=O)Nc5ccc(F)cc5)CC4)cc3)ccnc2cc1NCCC(=O)N[C@H](C(=O)N1C[C@H](O)C[C@H]1C(=O)NCc1ccc(-c2scnc2C)cc1)C(C)(C)C]

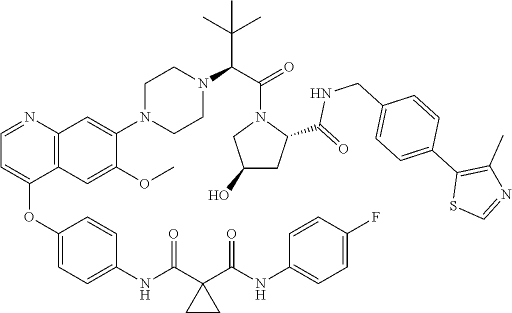 [SMILES: COc1cc2c(Oc3ccc(NC(=O)C4(C(=O)Nc5ccc(F)cc5)CC4)cc3)ccnc2cc1N1CCN([C@H](C(=O)N2C[C@H](O)C[C@H]2C(=O)NCc2ccc(-c3scnc3C)cc2)C(C)(C)C)CC1]

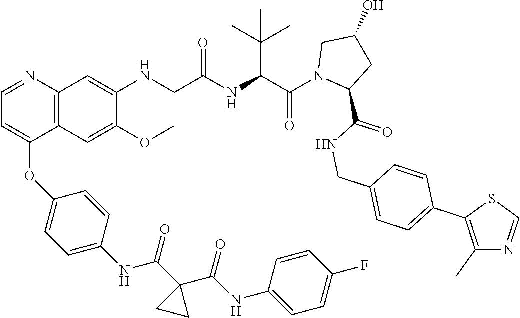 [SMILES: COc1cc2c(Oc3ccc(NC(=O)C4(C(=O)Nc5ccc(F)cc5)CC4)cc3)ccnc2cc1NCC(=O)N[C@H](C(=O)N1C[C@H](O)C[C@H]1C(=O)NCc1ccc(-c2scnc2C)cc1)C(C)(C)C]

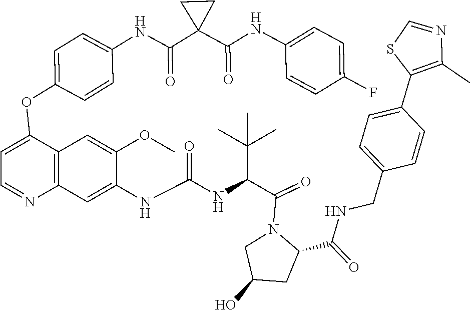 [SMILES: COc1cc2c(Oc3ccc(NC(=O)C4(C(=O)Nc5ccc(F)cc5)CC4)cc3)ccnc2cc1NC(=O)N[C@H](C(=O)N1C[C@H](O)C[C@H]1C(=O)NCc1ccc(-c2scnc2C)cc1)C(C)(C)C]

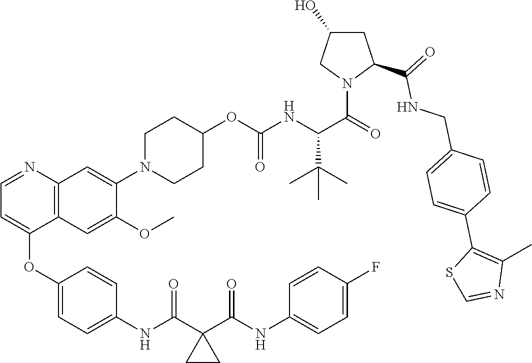 [SMILES: COc1cc2c(Oc3ccc(NC(=O)C4(C(=O)Nc5ccc(F)cc5)CC4)cc3)ccnc2cc1N1CCC(OC(=O)N[C@H](C(=O)N2C[C@H](O)C[C@H]2C(=O)NCc2ccc(-c3scnc3C)cc2)C(C)(C)C)CC1]

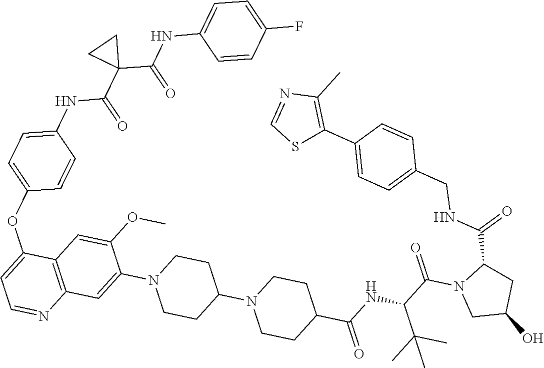 [SMILES: COc1cc2c(Oc3ccc(NC(=O)C4(C(=O)Nc5ccc(F)cc5)CC4)cc3)ccnc2cc1N1CCC(N2CCC(C(=O)N[C@H](C(=O)N3C[C@H](O)C[C@H]3C(=O)NCc3ccc(-c4scnc4C)cc3)C(C)(C)C)CC2)CC1]

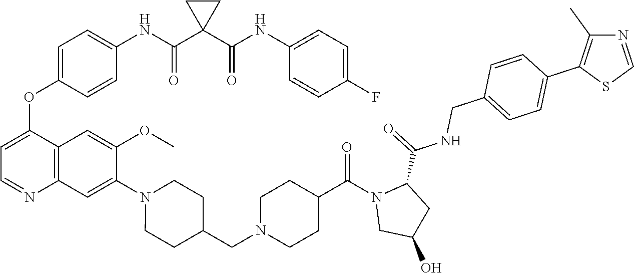 [SMILES: COc1cc2c(Oc3ccc(NC(=O)C4(C(=O)Nc5ccc(F)cc5)CC4)cc3)ccnc2cc1N1CCC(CN2CCC(C(=O)N3C[C@H](O)C[C@H]3C(=O)NCc3ccc(-c4scnc4C)cc3)CC2)CC1]

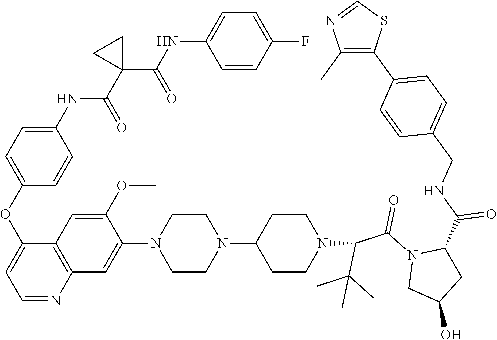 [SMILES: COc1cc2c(Oc3ccc(NC(=O)C4(C(=O)Nc5ccc(F)cc5)CC4)cc3)ccnc2cc1N1CCN(C2CCN([C@H](C(=O)N3C[C@H](O)C[C@H]3C(=O)NCc3ccc(-c4scnc4C)cc3)C(C)(C)C)CC2)CC1]

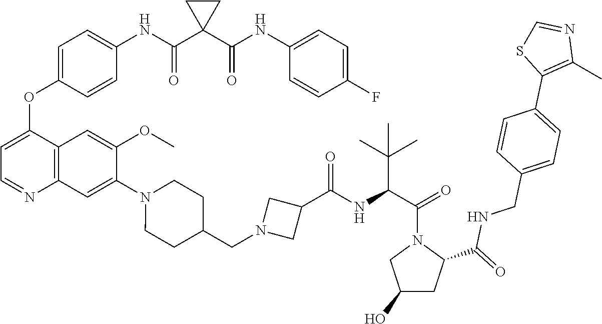 [SMILES: COc1cc2c(Oc3ccc(NC(=O)C4(C(=O)Nc5ccc(F)cc5)CC4)cc3)ccnc2cc1N1CCC(CN2CC(C(=O)N[C@H](C(=O)N3C[C@H](O)C[C@H]3C(=O)NCc3ccc(-c4scnc4C)cc3)C(C)(C)C)C2)CC1]

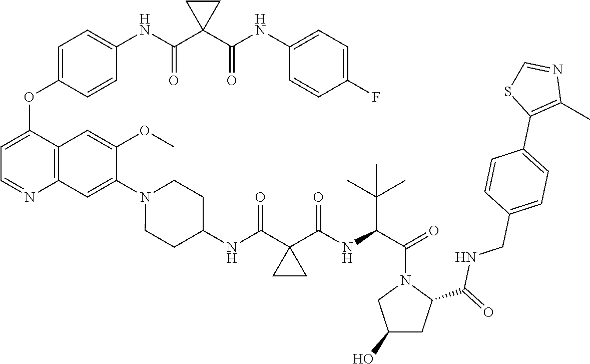 [SMILES: COc1cc2c(Oc3ccc(NC(=O)C4(C(=O)Nc5ccc(F)cc5)CC4)cc3)ccnc2cc1N1CCC(NC(=O)C2(C(=O)N[C@H](C(=O)N3C[C@H](O)C[C@H]3C(=O)NCc3ccc(-c4scnc4C)cc3)C(C)(C)C)CC2)CC1]

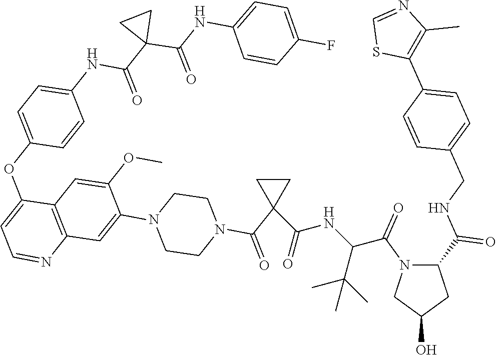 [SMILES: COc1cc2c(Oc3ccc(NC(=O)C4(C(=O)Nc5ccc(F)cc5)CC4)cc3)ccnc2cc1N1CCN(C(=O)C2(C(=O)NC(C(=O)N3C[C@H](O)C[C@H]3C(=O)NCc3ccc(-c4scnc4C)cc3)C(C)(C)C)CC2)CC1]

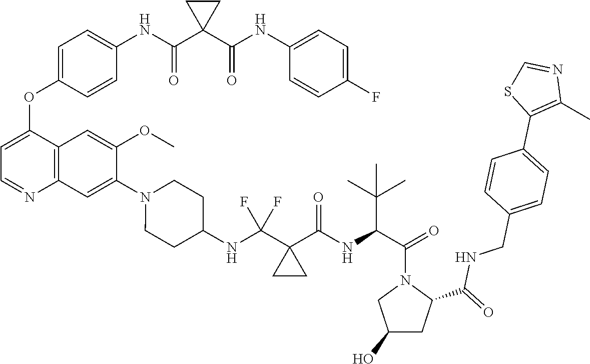 [SMILES: COc1cc2c(Oc3ccc(NC(=O)C4(C(=O)Nc5ccc(F)cc5)CC4)cc3)ccnc2cc1N1CCC(NC(F)(F)C2(C(=O)N[C@H](C(=O)N3C[C@H](O)C[C@H]3C(=O)NCc3ccc(-c4scnc4C)cc3)C(C)(C)C)CC2)CC1]

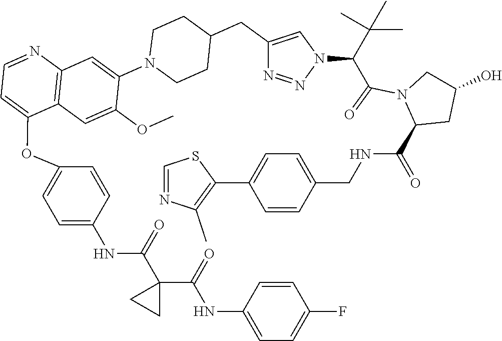 [SMILES: COc1cc2c(Oc3ccc(NC(=O)C4(C(=O)Nc5ccc(F)cc5)CC4)cc3)ccnc2cc1N1CCC(Cc2cn([C@H](C(=O)N3C[C@H](O)C[C@H]3C(=O)NCc3ccc(-c4scnc4C)cc3)C(C)(C)C)nn2)CC1]

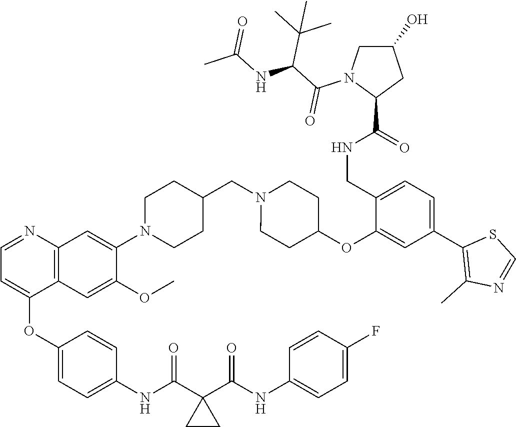 [SMILES: COc1cc2c(Oc3ccc(NC(=O)C4(C(=O)Nc5ccc(F)cc5)CC4)cc3)ccnc2cc1N1CCC(CN2CCC(Oc3cc(-c4scnc4C)ccc3CNC(=O)[C@@H]3C[C@@H](O)CN3C(=O)[C@@H](NC(C)=O)C(C)(C)C)CC2)CC1]

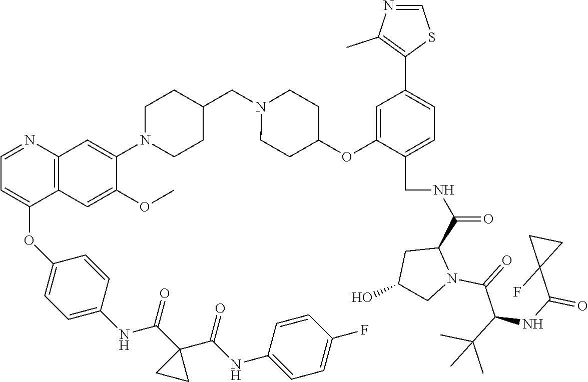 [SMILES: COc1cc2c(Oc3ccc(NC(=O)C4(C(=O)Nc5ccc(F)cc5)CC4)cc3)ccnc2cc1N1CCC(CN2CCC(Oc3cc(-c4scnc4C)ccc3CNC(=O)[C@@H]3C[C@@H](O)CN3C(=O)[C@@H](NC(=O)C3(F)CC3)C(C)(C)C)CC2)CC1]

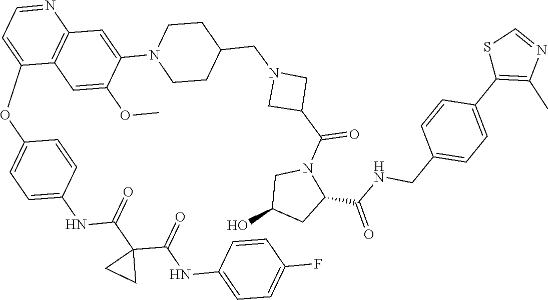 [SMILES: COc1cc2c(Oc3ccc(NC(=O)C4(C(=O)Nc5ccc(F)cc5)CC4)cc3)ccnc2cc1N1CCC(CN2CC(C(=O)N3C[C@H](O)C[C@H]3C(=O)NCc3ccc(-c4scnc4C)cc3)C2)CC1]

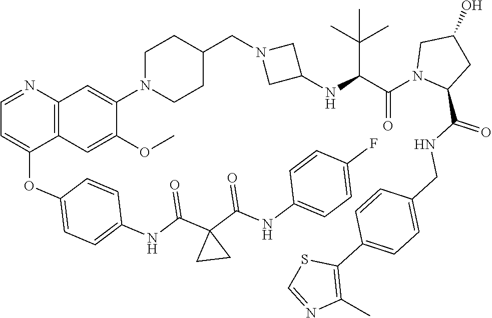 [SMILES: COc1cc2c(Oc3ccc(NC(=O)C4(C(=O)Nc5ccc(F)cc5)CC4)cc3)ccnc2cc1N1CCC(CN2CC(N[C@H](C(=O)N3C[C@H](O)C[C@H]3C(=O)NCc3ccc(-c4scnc4C)cc3)C(C)(C)C)C2)CC1]

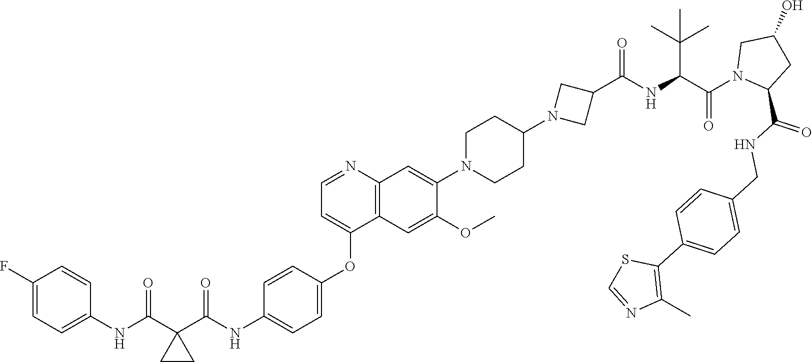 [SMILES: COc1cc2c(Oc3ccc(NC(=O)C4(C(=O)Nc5ccc(F)cc5)CC4)cc3)ccnc2cc1N1CCC(N2CC(C(=O)N[C@H](C(=O)N3C[C@H](O)C[C@H]3C(=O)NCc3ccc(-c4scnc4C)cc3)C(C)(C)C)C2)CC1]

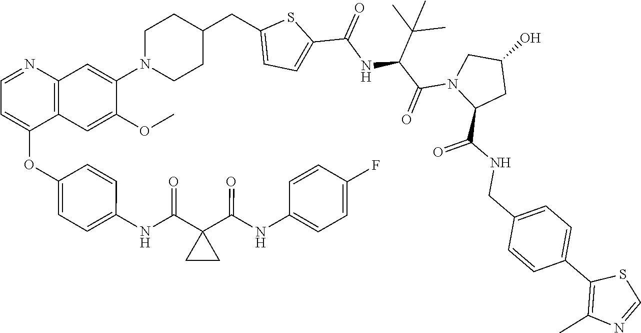 [SMILES: COc1cc2c(Oc3ccc(NC(=O)C4(C(=O)Nc5ccc(F)cc5)CC4)cc3)ccnc2cc1N1CCC(Cc2ccc(C(=O)N[C@H](C(=O)N3C[C@H](O)C[C@H]3C(=O)NCc3ccc(-c4scnc4C)cc3)C(C)(C)C)s2)CC1]

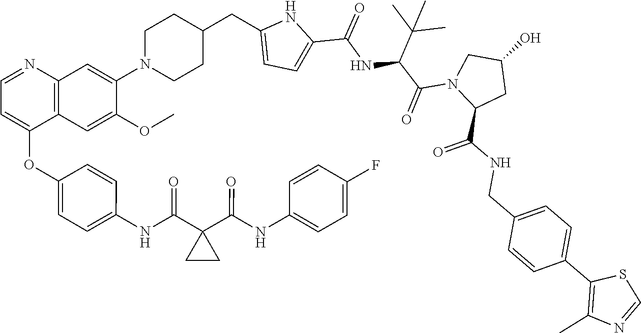 [SMILES: COc1cc2c(Oc3ccc(NC(=O)C4(C(=O)Nc5ccc(F)cc5)CC4)cc3)ccnc2cc1N1CCC(Cc2ccc(C(=O)N[C@H](C(=O)N3C[C@H](O)C[C@H]3C(=O)NCc3ccc(-c4scnc4C)cc3)C(C)(C)C)[nH]2)CC1]

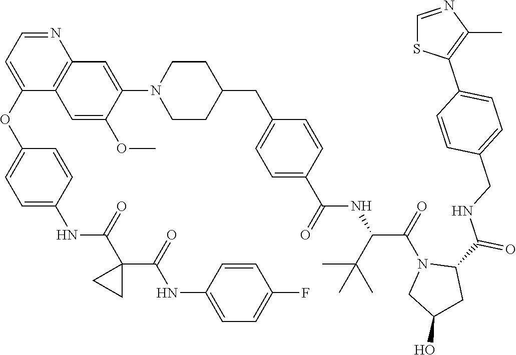 [SMILES: COc1cc2c(Oc3ccc(NC(=O)C4(C(=O)Nc5ccc(F)cc5)CC4)cc3)ccnc2cc1N1CCC(Cc2ccc(C(=O)N[C@H](C(=O)N3C[C@H](O)C[C@H]3C(=O)NCc3ccc(-c4scnc4C)cc3)C(C)(C)C)cc2)CC1]

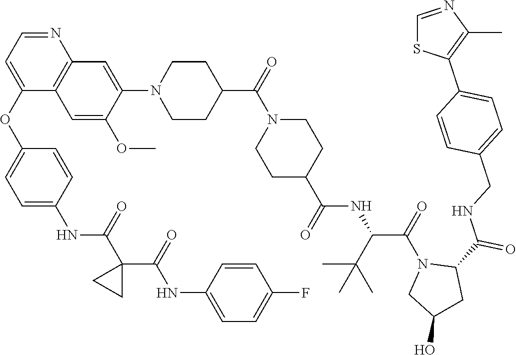 [SMILES: COc1cc2c(Oc3ccc(NC(=O)C4(C(=O)Nc5ccc(F)cc5)CC4)cc3)ccnc2cc1N1CCC(C(=O)N2CCC(C(=O)N[C@H](C(=O)N3C[C@H](O)C[C@H]3C(=O)NCc3ccc(-c4scnc4C)cc3)C(C)(C)C)CC2)CC1]

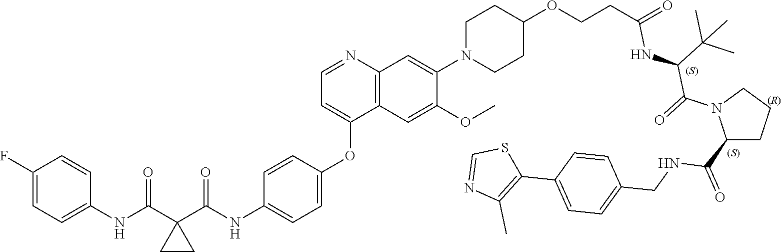

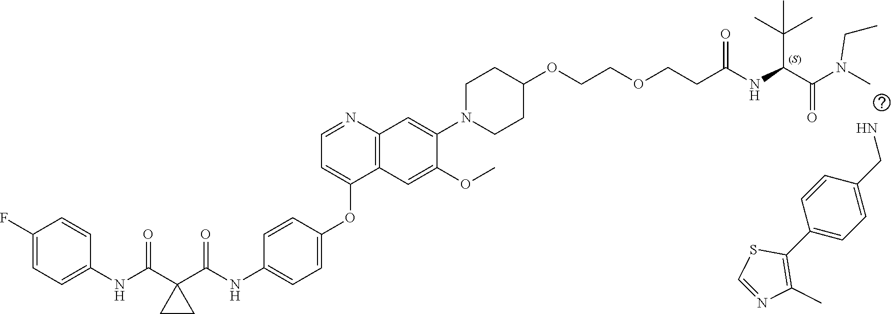

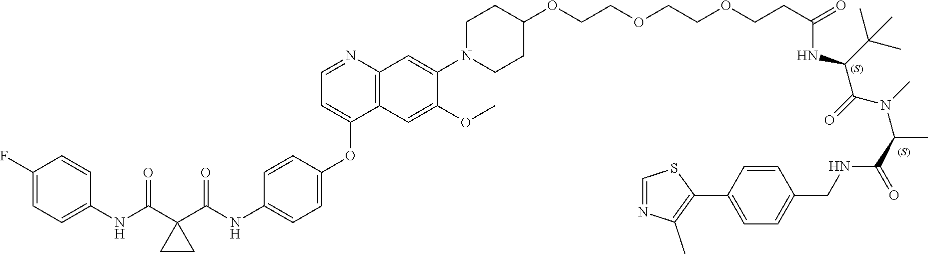

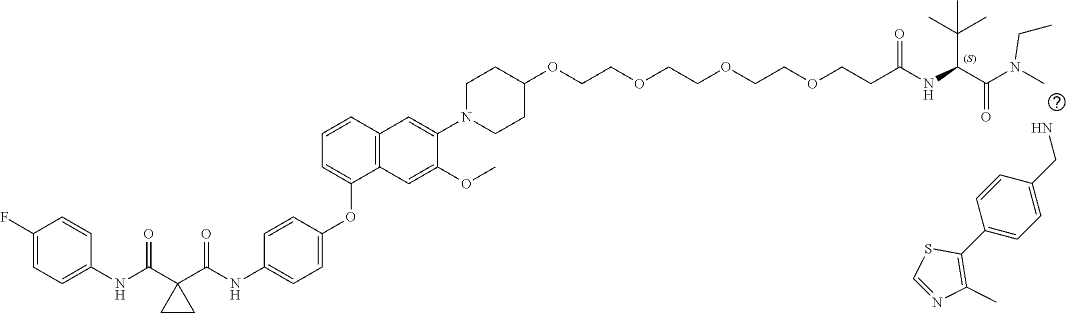

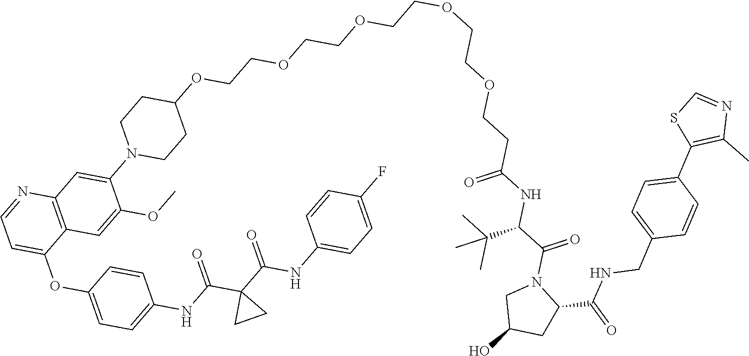 [SMILES: COc1cc2c(Oc3ccc(NC(=O)C4(C(=O)Nc5ccc(F)cc5)CC4)cc3)ccnc2cc1N1CCC(OCCOCCOCCOCCOCCC(=O)N[C@H](C(=O)N2C[C@H](O)C[C@H]2C(=O)NCc2ccc(-c3scnc3C)cc2)C(C)(C)C)CC1]

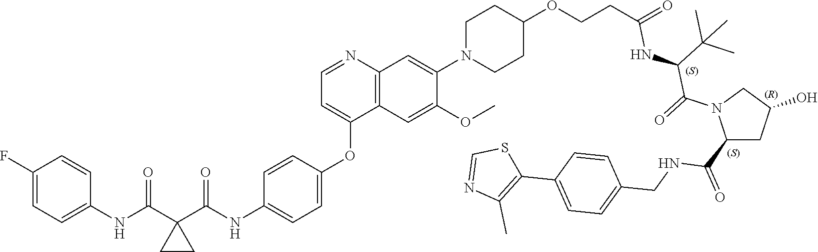

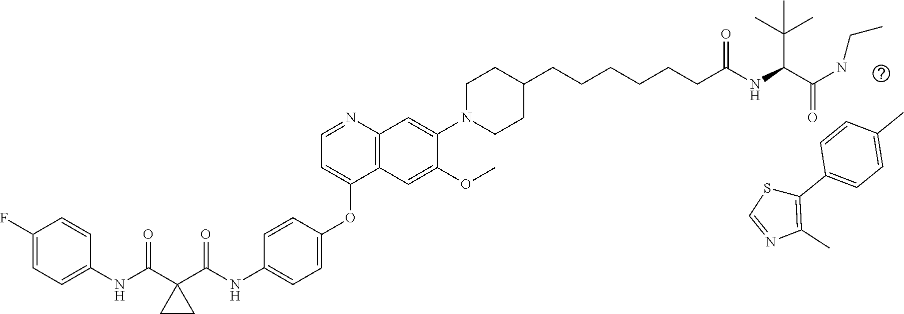

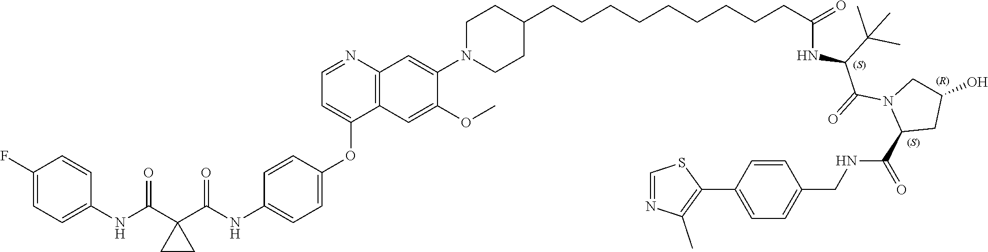

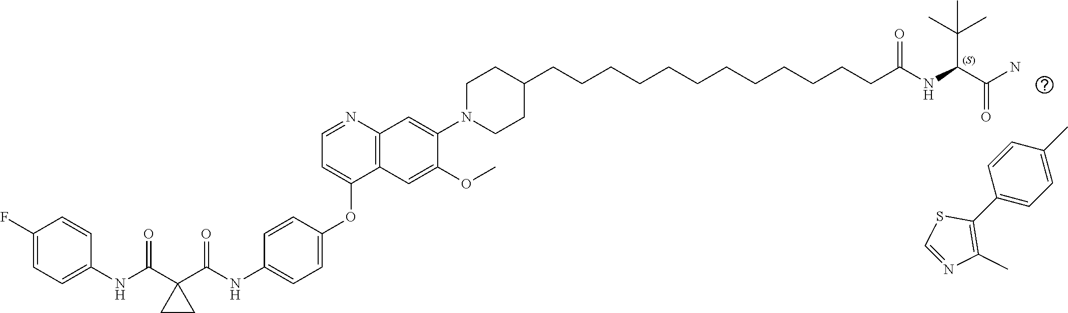

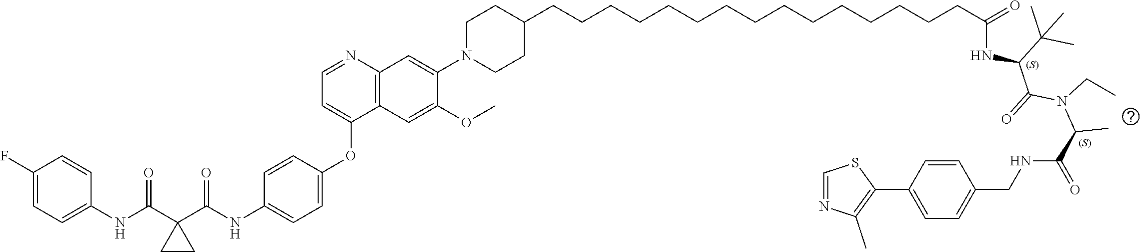

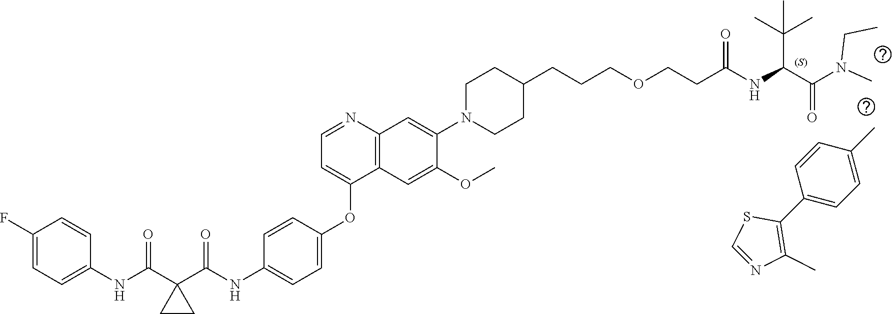

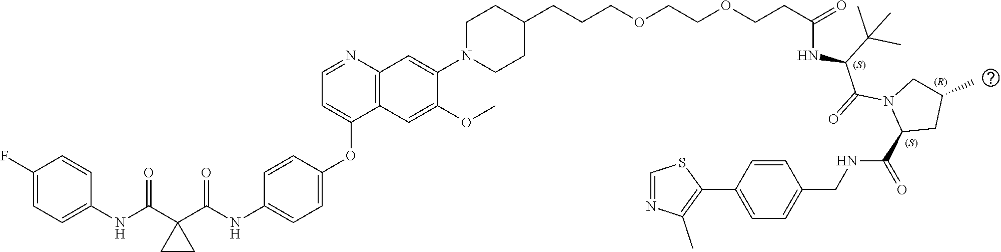

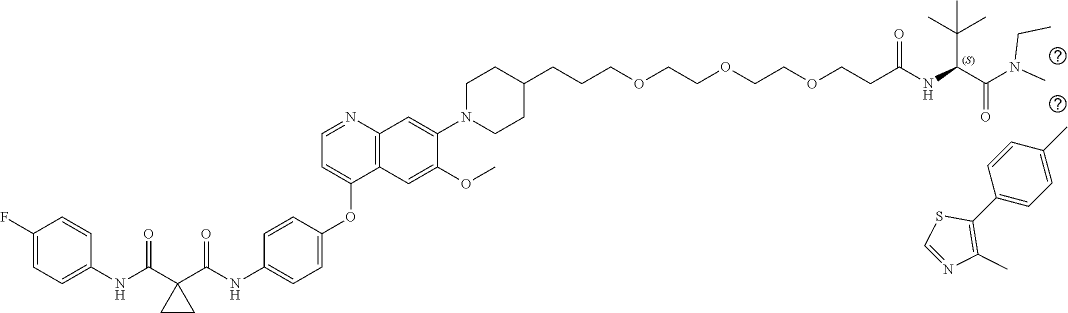

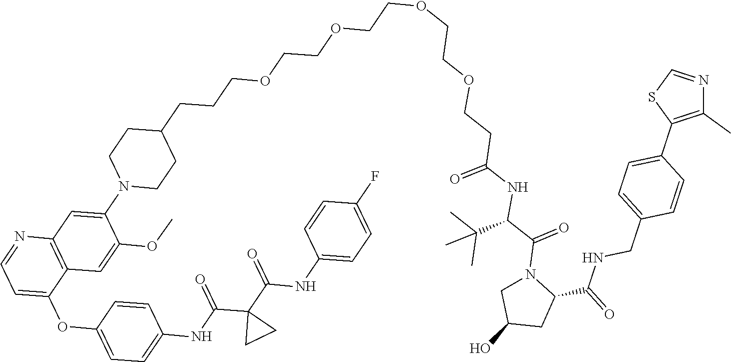 [SMILES: COc1cc2c(Oc3ccc(NC(=O)C4(C(=O)Nc5ccc(F)cc5)CC4)cc3)ccnc2cc1N1CCC(CCCOCCOCCOCCOCCC(=O)N[C@H](C(=O)N2C[C@H](O)C[C@H]2C(=O)NCc2ccc(-c3scnc3C)cc2)C(C)(C)C)CC1]

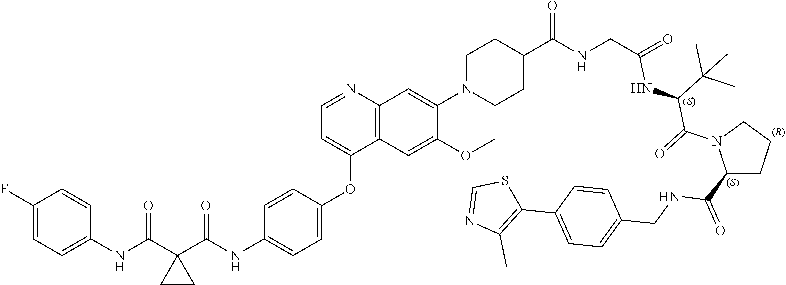

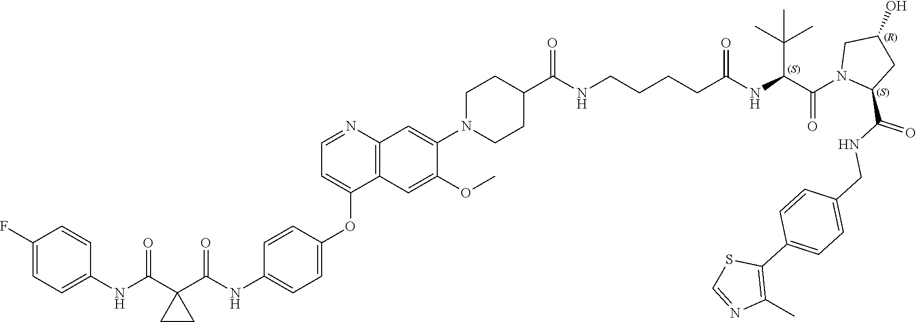

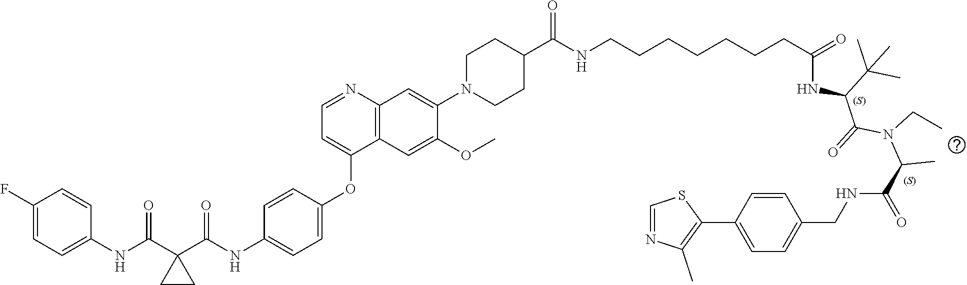

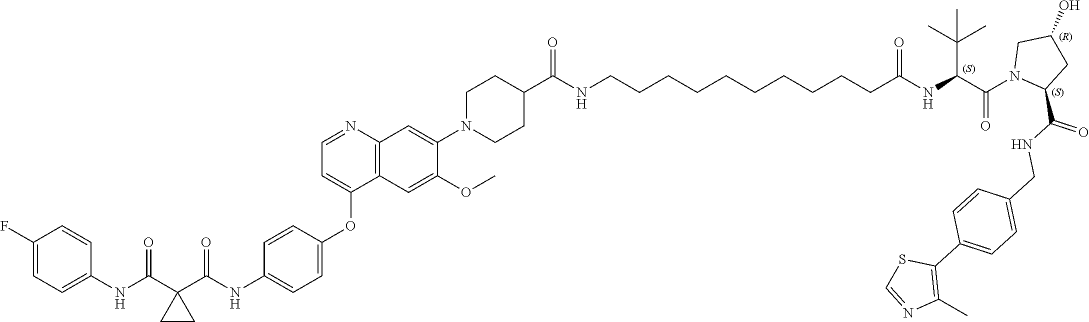

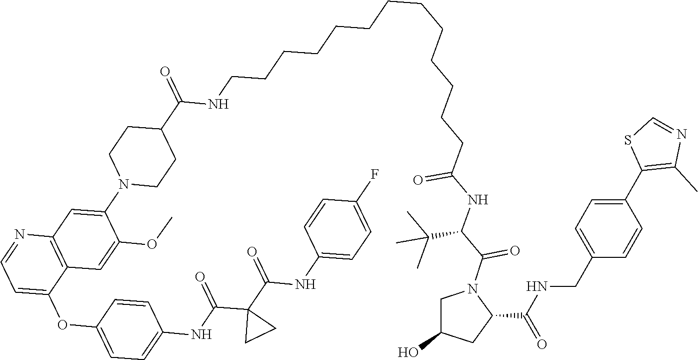 [SMILES: COc1cc2c(Oc3ccc(NC(=O)C4(C(=O)Nc5ccc(F)cc5)CC4)cc3)ccnc2cc1N1CCC(C(=O)NCCCCCCCCCCCCCC(=O)N[C@H](C(=O)N2C[C@H](O)C[C@H]2C(=O)NCc2ccc(-c3scnc3C)cc2)C(C)(C)C)CC1]

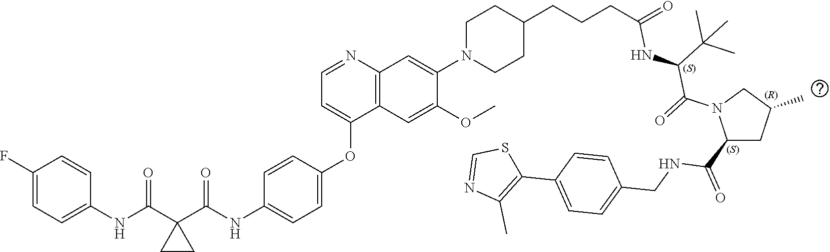

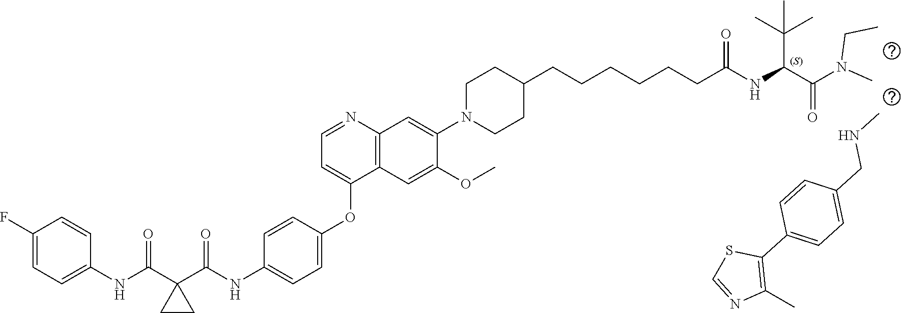

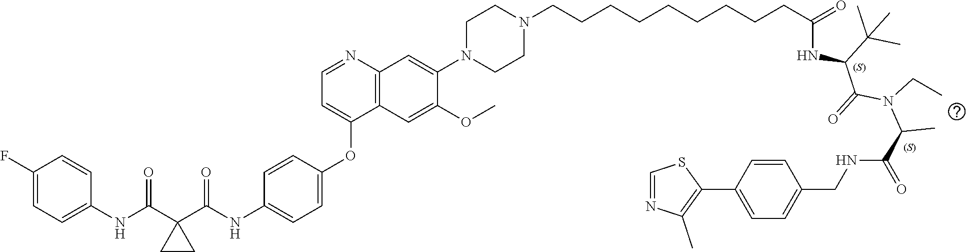

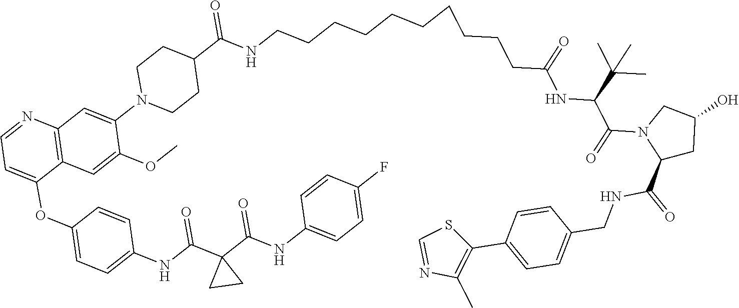 [SMILES: COc1cc2c(Oc3ccc(NC(=O)C4(C(=O)Nc5ccc(F)cc5)CC4)cc3)ccnc2cc1N1CCC(C(=O)NCCCCCCCCCCC(=O)N[C@H](C(=O)N2C[C@H](O)C[C@H]2C(=O)NCc2ccc(-c3scnc3C)cc2)C(C)(C)C)CC1]

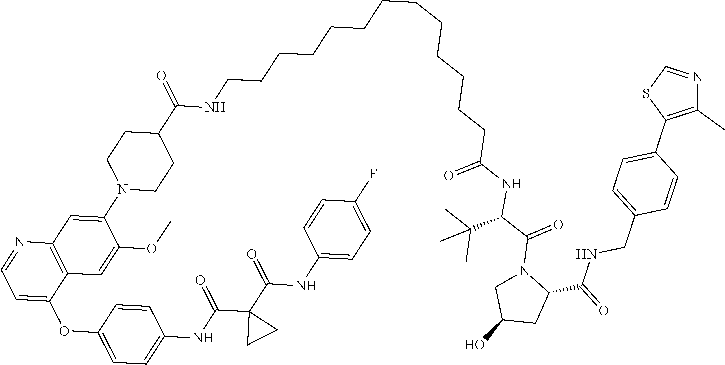 [SMILES: COc1cc2c(Oc3ccc(NC(=O)C4(C(=O)Nc5ccc(F)cc5)CC4)cc3)ccnc2cc1N1CCC(C(=O)NCCCCCCCCCCCCCC(=O)N[C@H](C(=O)N2C[C@H](O)C[C@H]2C(=O)NCc2ccc(-c3scnc3C)cc2)C(C)(C)C)CC1]

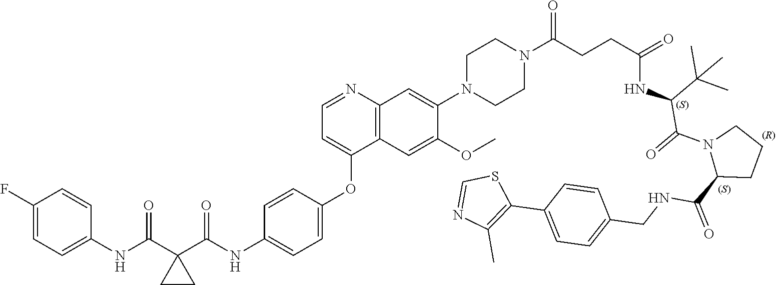

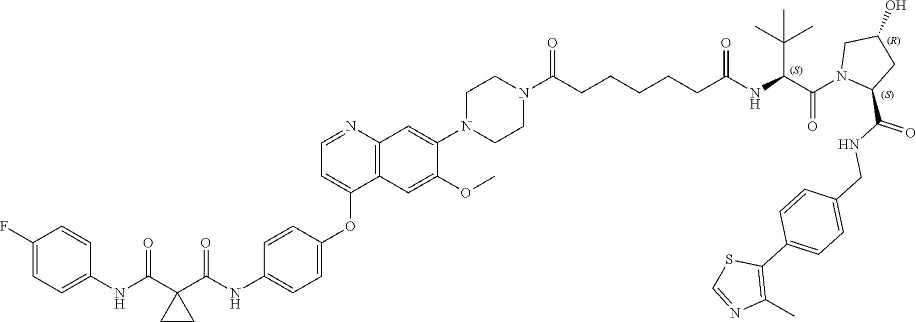

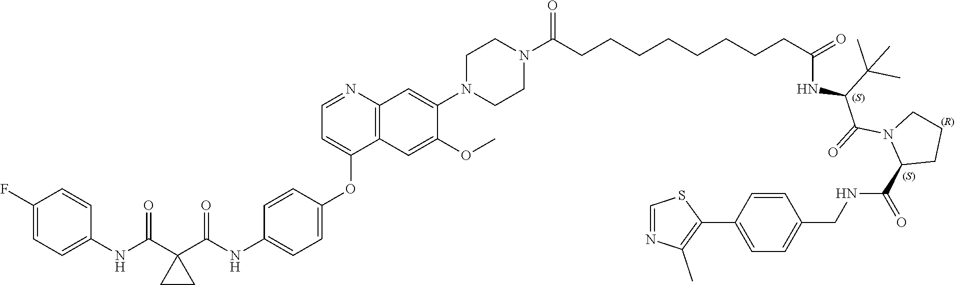

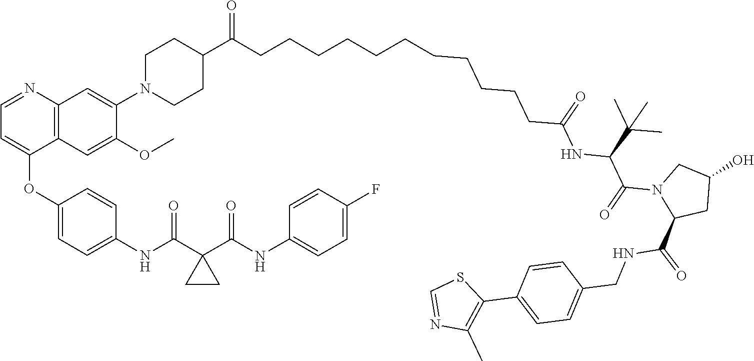 [SMILES: COc1cc2c(Oc3ccc(NC(=O)C4(C(=O)Nc5ccc(F)cc5)CC4)cc3)ccnc2cc1N1CCC(C(=O)CCCCCCCCCCCC(=O)N[C@H](C(=O)N2C[C@H](O)C[C@H]2C(=O)NCc2ccc(-c3scnc3C)cc2)C(C)(C)C)CC1]

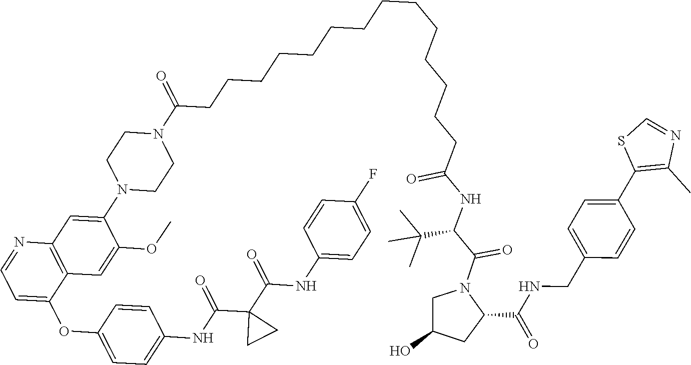 [SMILES: COc1cc2c(Oc3ccc(NC(=O)C4(C(=O)Nc5ccc(F)cc5)CC4)cc3)ccnc2cc1N1CCN(C(=O)CCCCCCCCCCCCCCC(=O)N[C@H](C(=O)N2C[C@H](O)C[C@H]2C(=O)NCc2ccc(-c3scnc3C)cc2)C(C)(C)C)CC1]

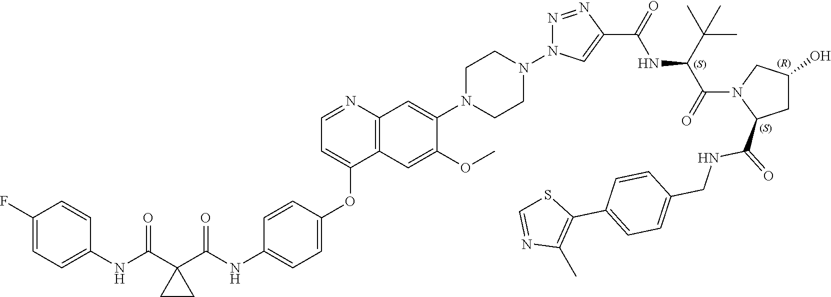

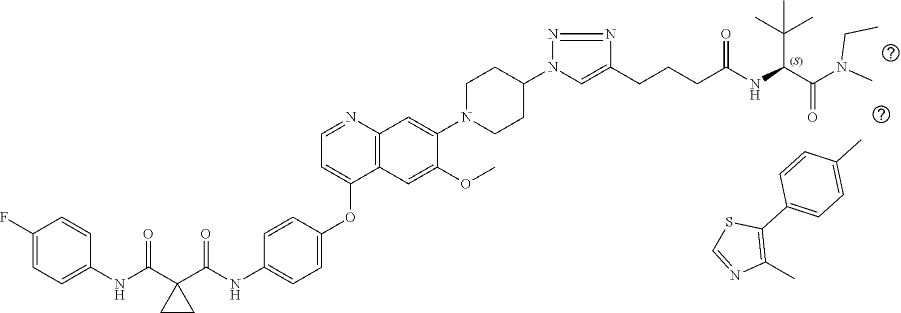

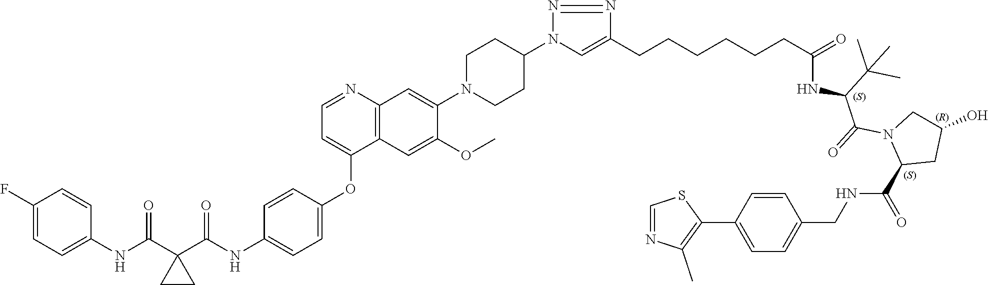

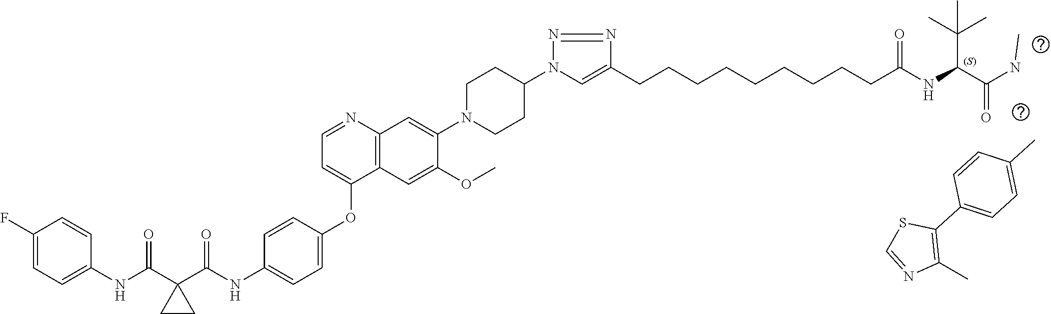

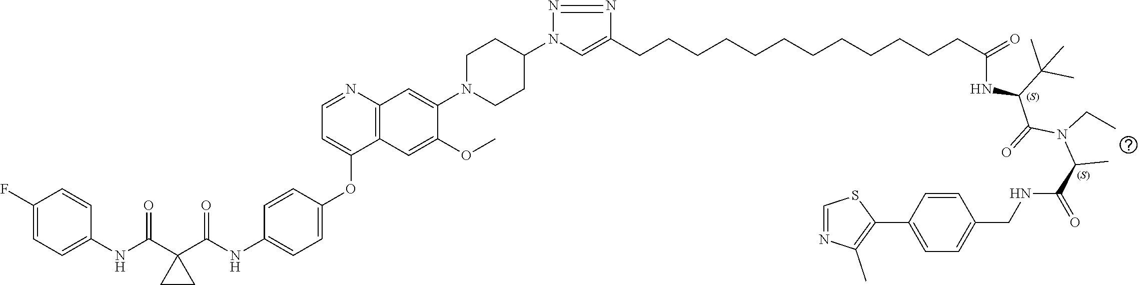

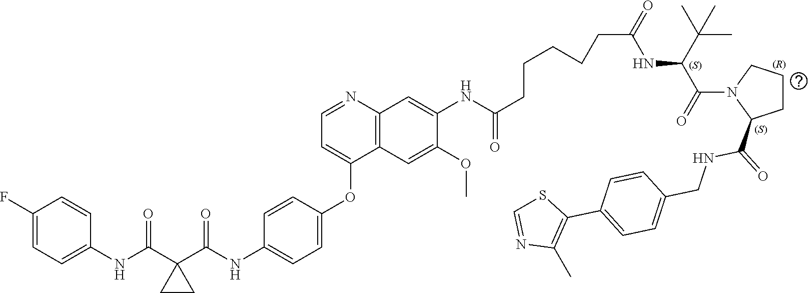

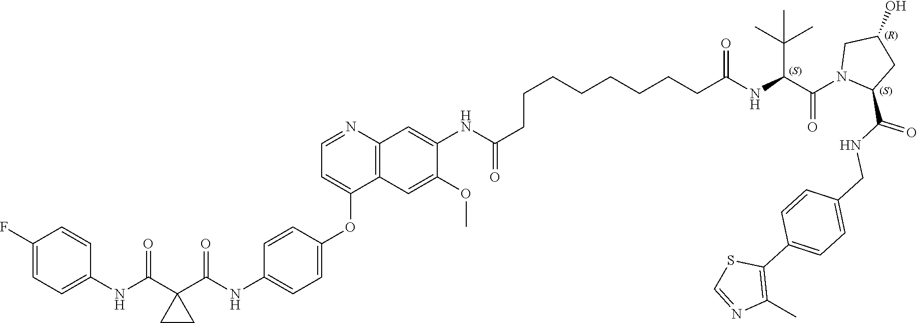

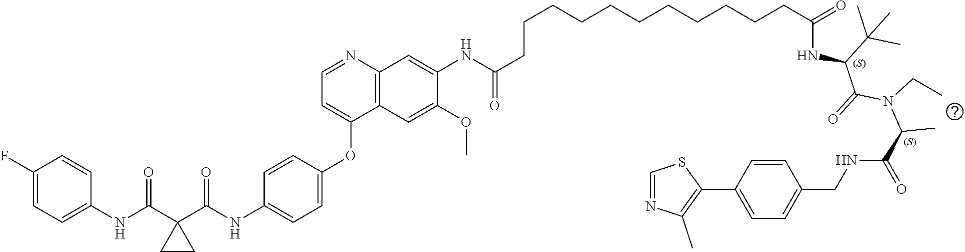

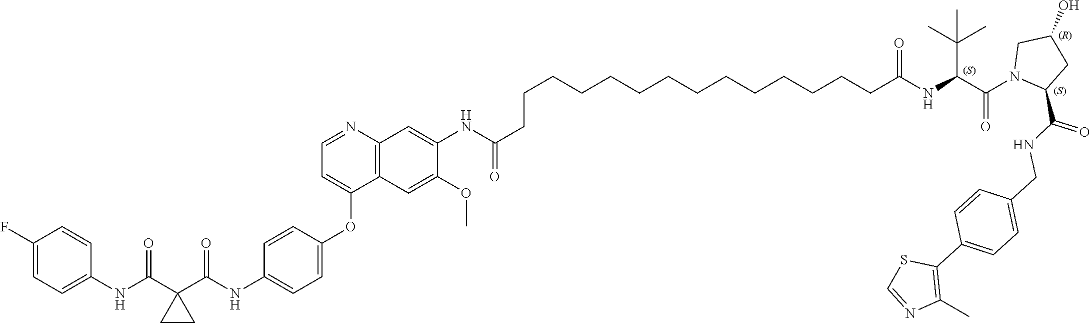

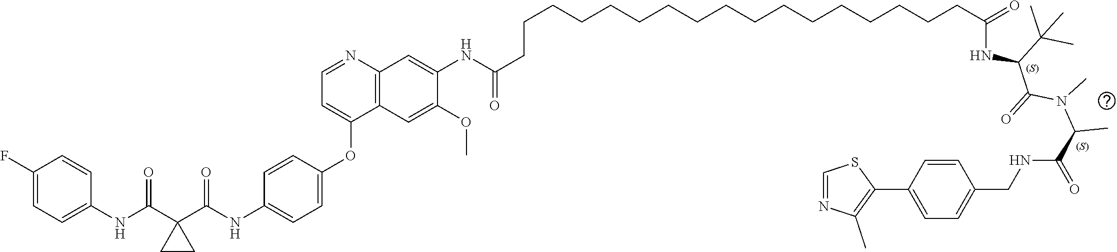

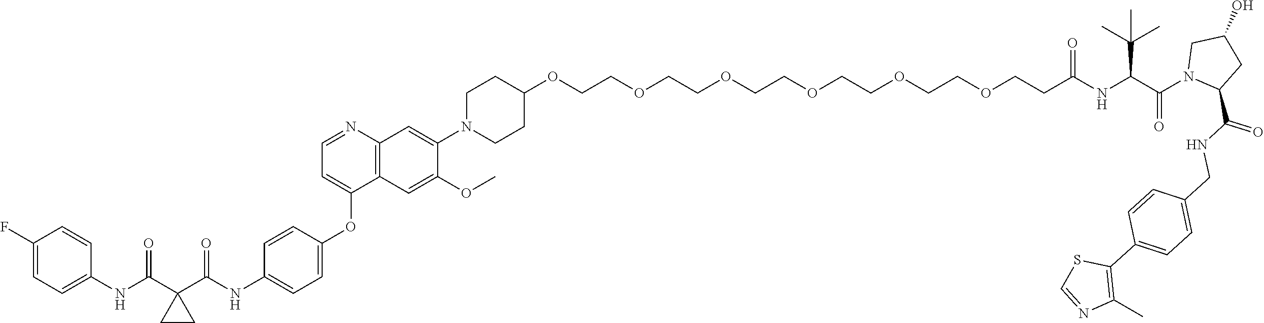 [SMILES: COc1cc2c(Oc3ccc(NC(=O)C4(C(=O)Nc5ccc(F)cc5)CC4)cc3)ccnc2cc1N1CCC(OCCOCCOCCOCCOCCOCCC(=O)N[C@H](C(=O)N2C[C@H](O)C[C@H]2C(=O)NCc2ccc(-c3scnc3C)cc2)C(C)(C)C)CC1]

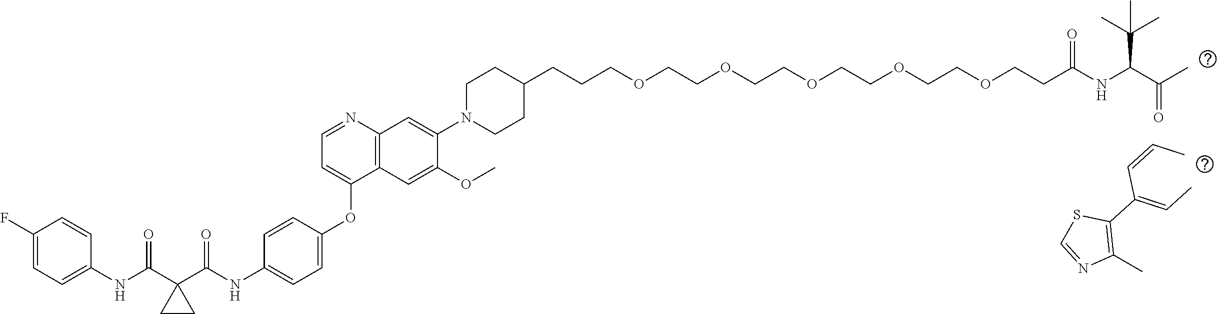

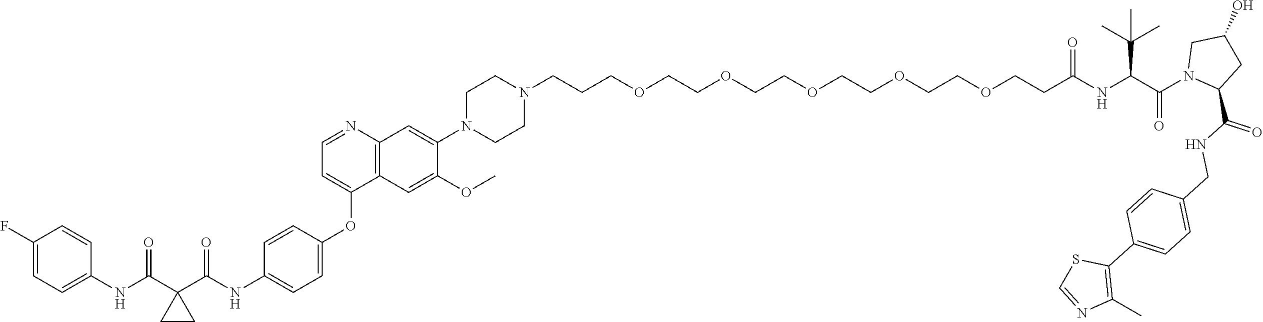 [SMILES: COc1cc2c(Oc3ccc(NC(=O)C4(C(=O)Nc5ccc(F)cc5)CC4)cc3)ccnc2cc1N1CCN(CCCOCCOCCOCCOCCOCCC(=O)N[C@H](C(=O)N2C[C@H](O)C[C@H]2C(=O)NCc2ccc(-c3scnc3C)cc2)C(C)(C)C)CC1]

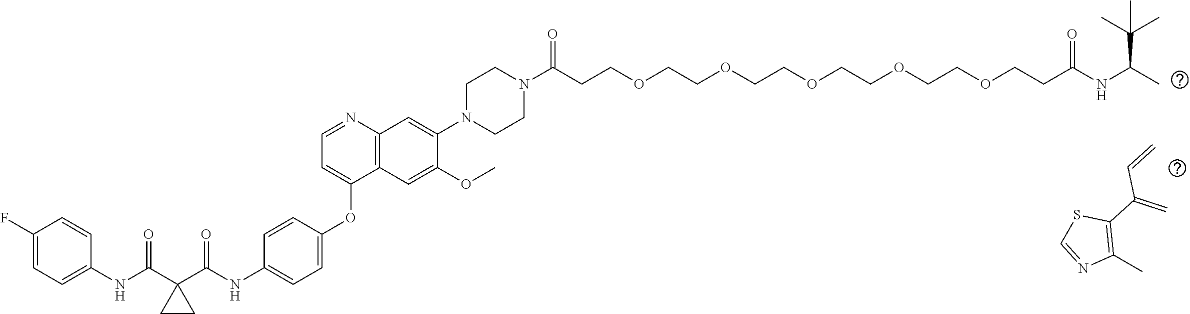

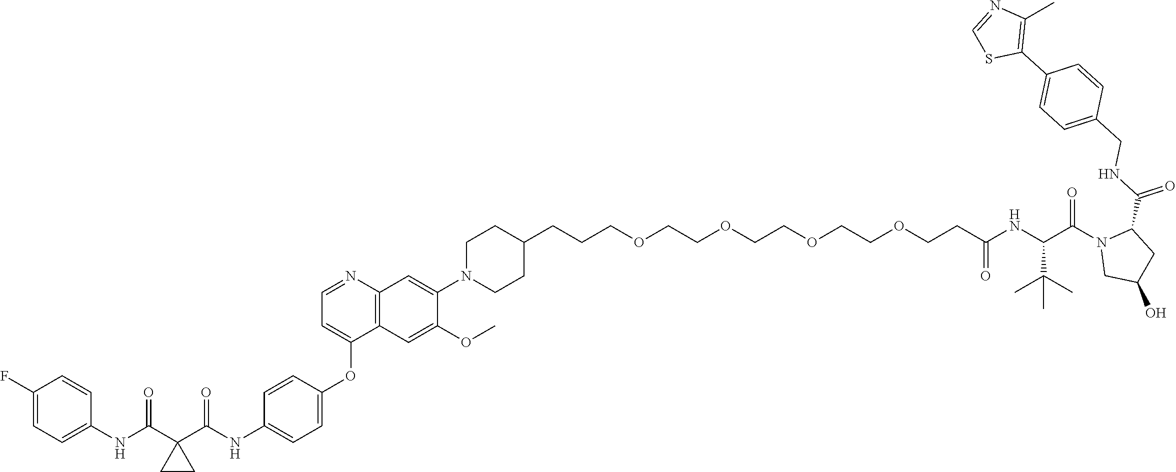 [SMILES: COc1cc2c(Oc3ccc(NC(=O)C4(C(=O)Nc5ccc(F)cc5)CC4)cc3)ccnc2cc1N1CCC(CCCOCCOCCOCCOCCC(=O)N[C@H](C(=O)N2C[C@H](O)C[C@H]2C(=O)NCc2ccc(-c3scnc3C)cc2)C(C)(C)C)CC1]

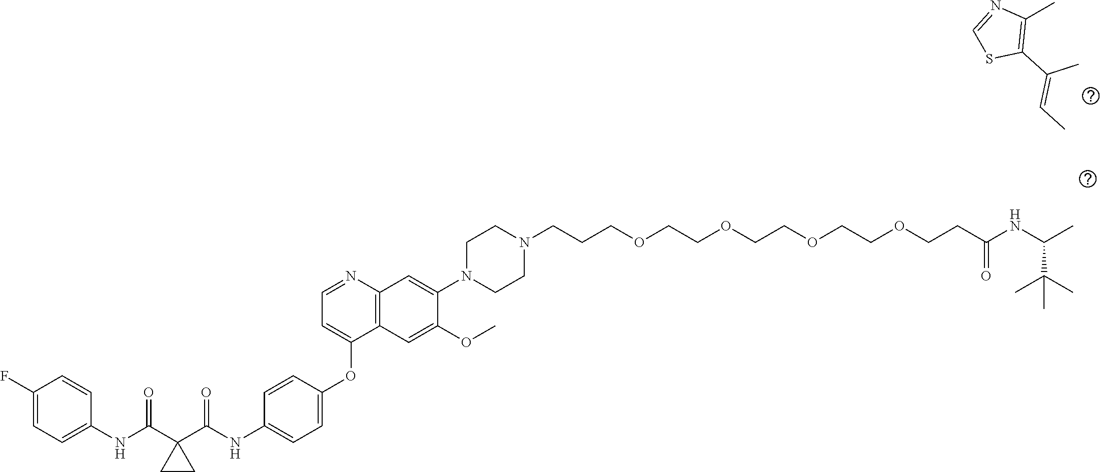

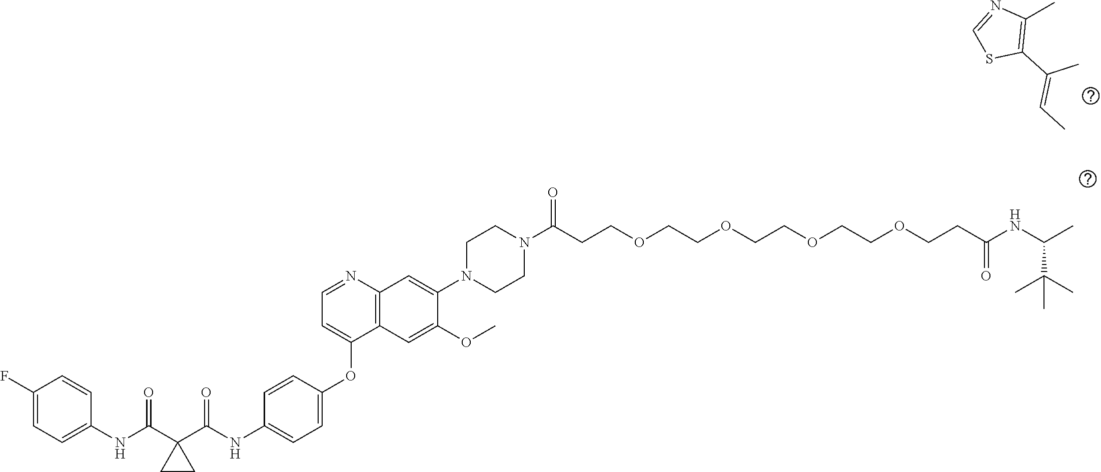

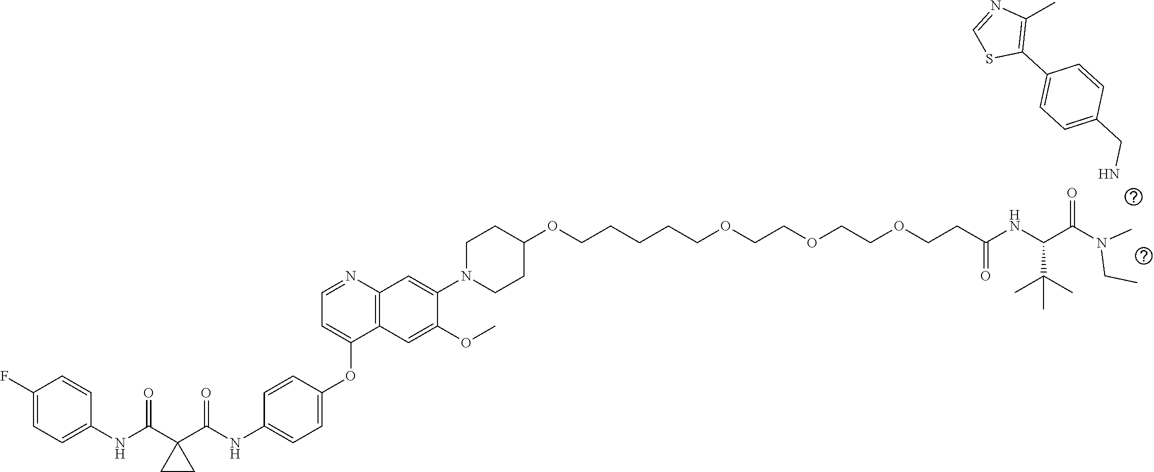

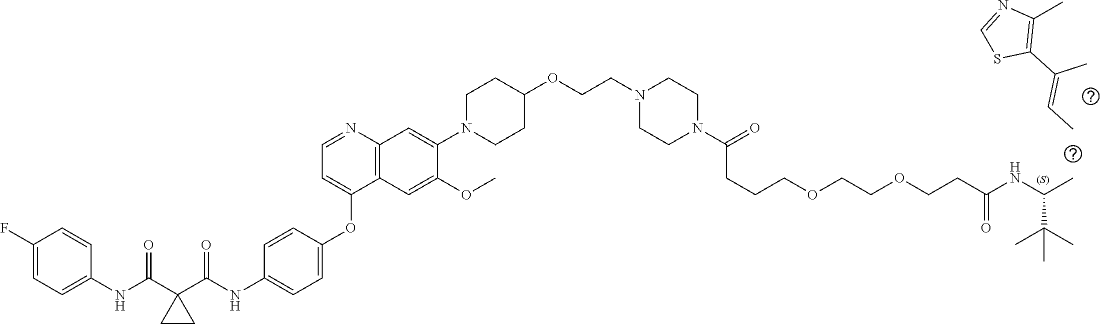

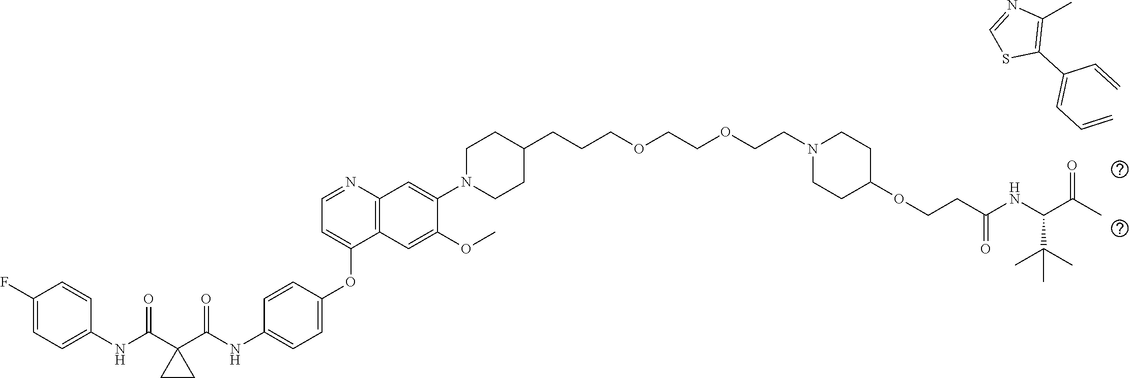

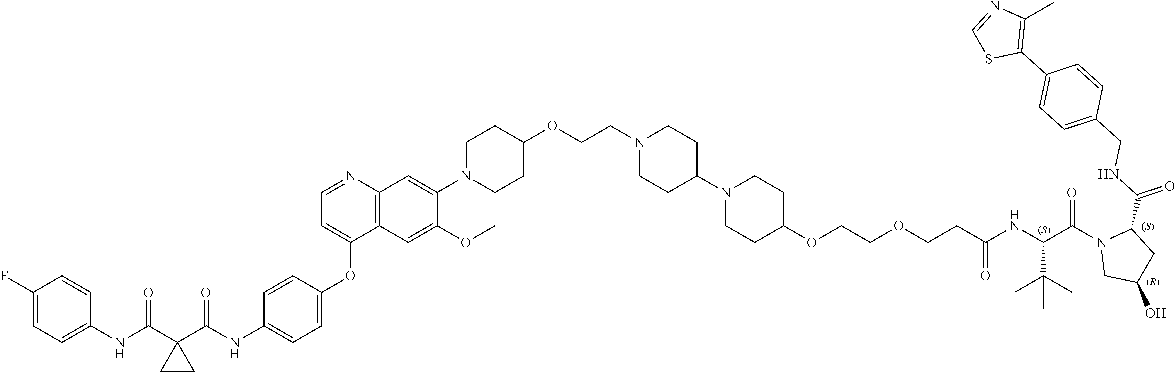

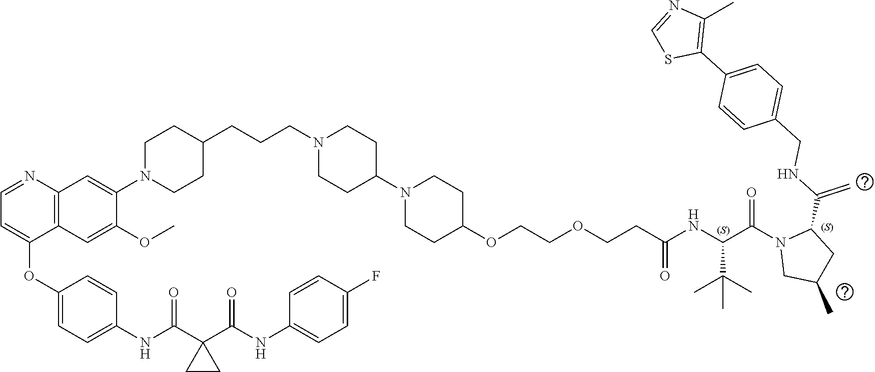

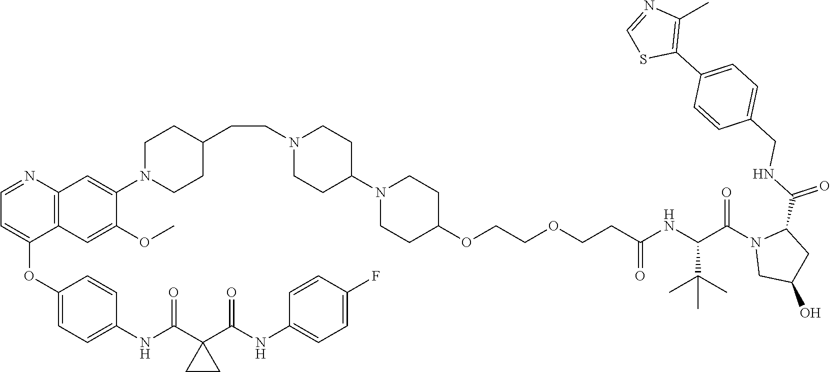 [SMILES: COc1cc2c(Oc3ccc(NC(=O)C4(C(=O)Nc5ccc(F)cc5)CC4)cc3)ccnc2cc1N1CCC(CCN2CCC(N3CCC(OCCOCCC(=O)N[C@H](C(=O)N4C[C@H](O)C[C@H]4C(=O)NCc4ccc(-c5scnc5C)cc4)C(C)(C)C)CC3)CC2)CC1]

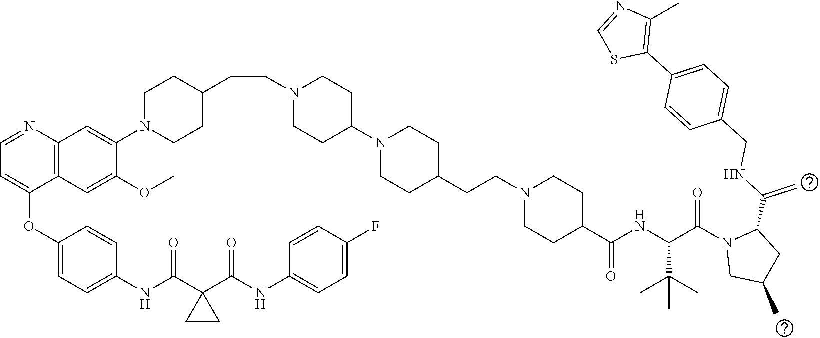 [SMILES: C=C(NCc1ccc(-c2scnc2C)cc1)[C@@H]1C[C@@H](C)CN1C(=O)[C@@H](NC(=O)C1CCN(CCC2CCN(C3CCN(CCC4CCN(c5cc6nccc(Oc7ccc(NC(=O)C8(C(=O)Nc9ccc(F)cc9)CC8)cc7)c6cc5OC)CC4)CC3)CC2)CC1)C(C)(C)C]

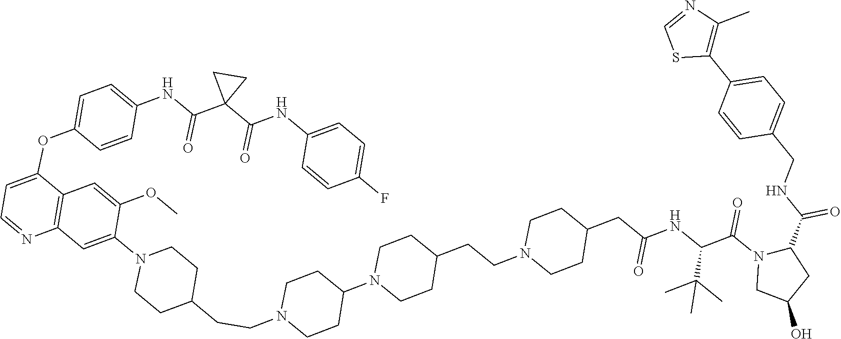

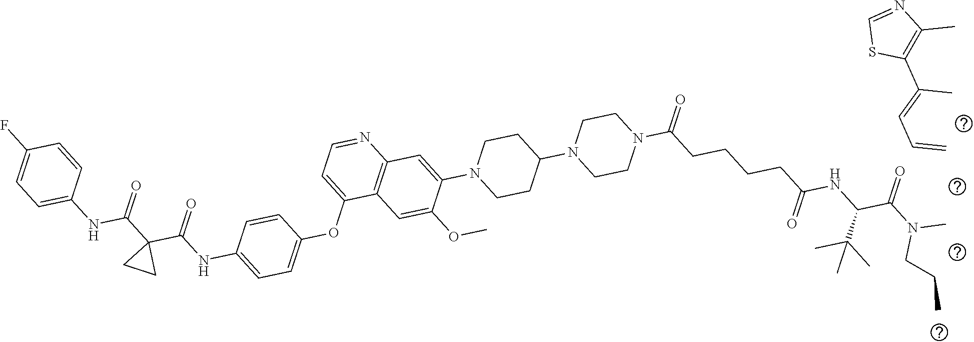

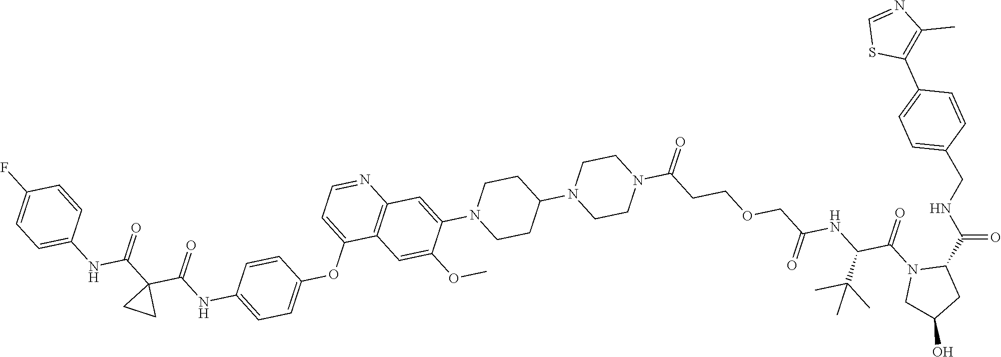 [SMILES: COc1cc2c(Oc3ccc(NC(=O)C4(C(=O)Nc5ccc(F)cc5)CC4)cc3)ccnc2cc1N1CCC(N2CCN(C(=O)CCOCC(=O)N[C@H](C(=O)N3C[C@H](O)C[C@H]3C(=O)NCc3ccc(-c4scnc4C)cc3)C(C)(C)C)CC2)CC1]

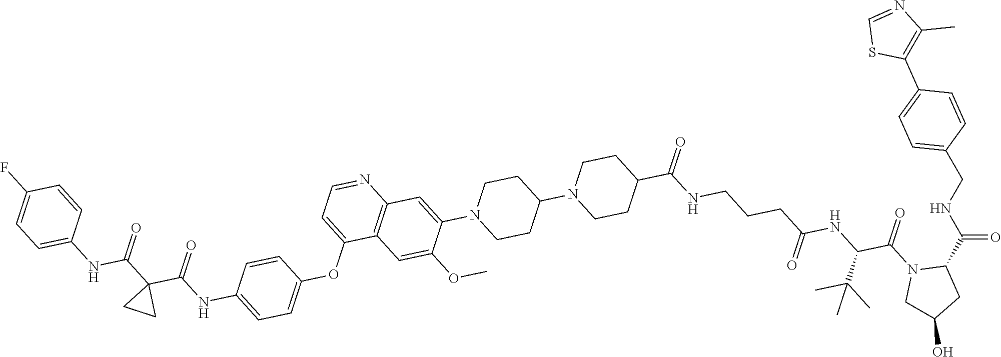 [SMILES: COc1cc2c(Oc3ccc(NC(=O)C4(C(=O)Nc5ccc(F)cc5)CC4)cc3)ccnc2cc1N1CCC(N2CCC(C(=O)NCCCC(=O)N[C@H](C(=O)N3C[C@H](O)C[C@H]3C(=O)NCc3ccc(-c4scnc4C)cc3)C(C)(C)C)CC2)CC1]

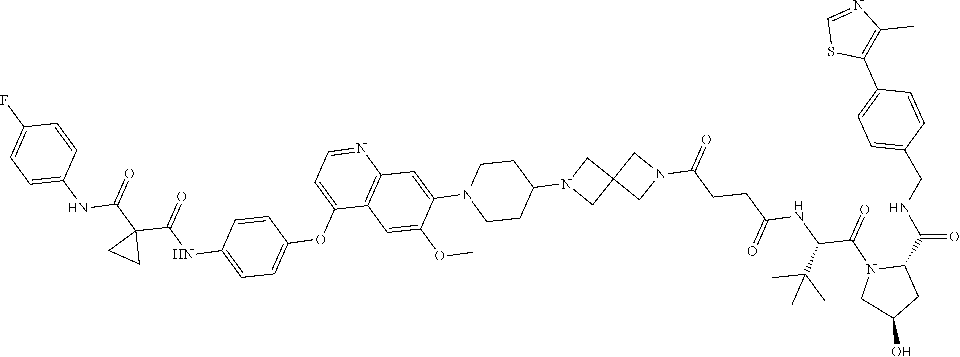 [SMILES: COc1cc2c(Oc3ccc(NC(=O)C4(C(=O)Nc5ccc(F)cc5)CC4)cc3)ccnc2cc1N1CCC(N2CC3(CN(C(=O)CCC(=O)N[C@H](C(=O)N4C[C@H](O)C[C@H]4C(=O)NCc4ccc(-c5scnc5C)cc4)C(C)(C)C)C3)C2)CC1]

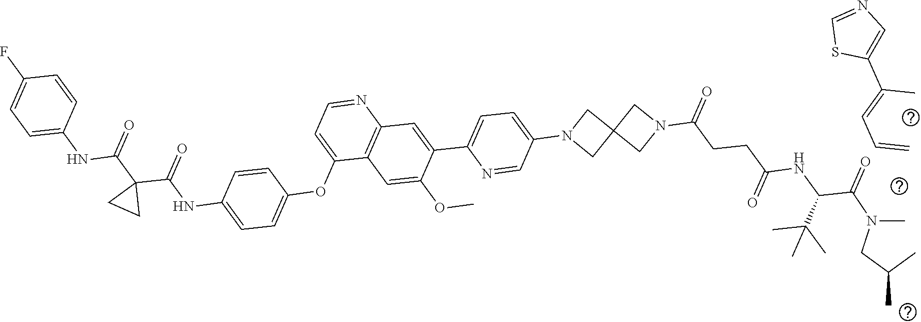

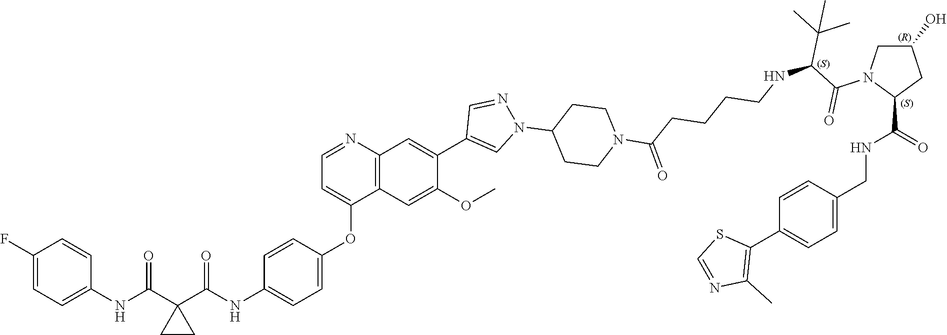

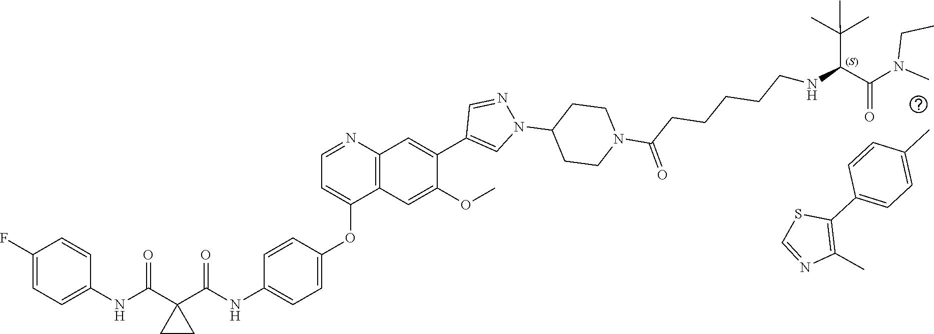

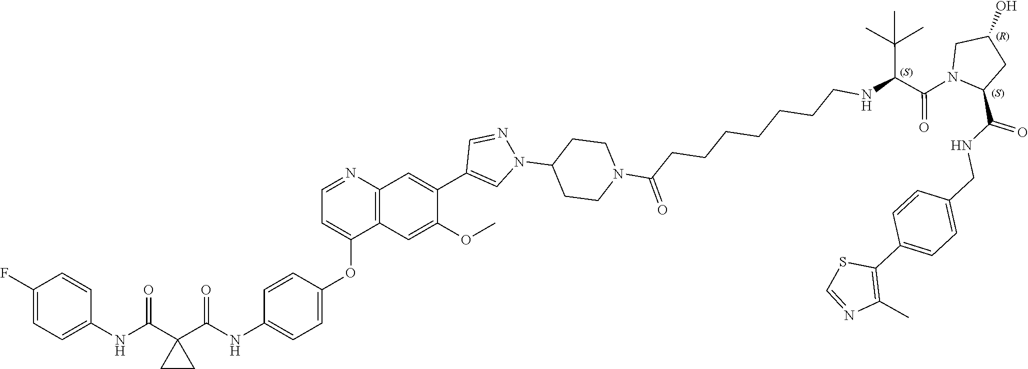

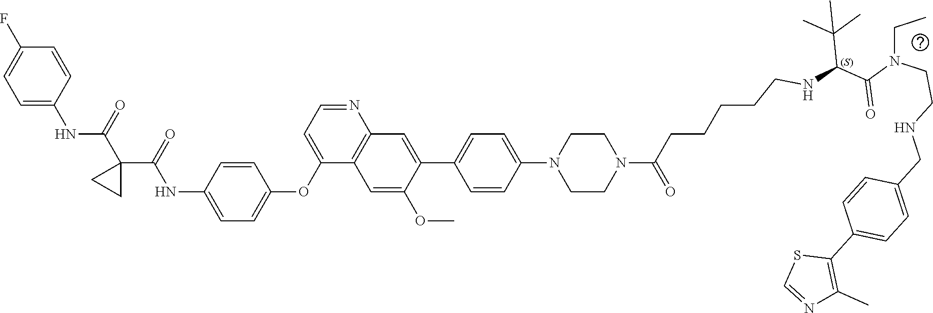

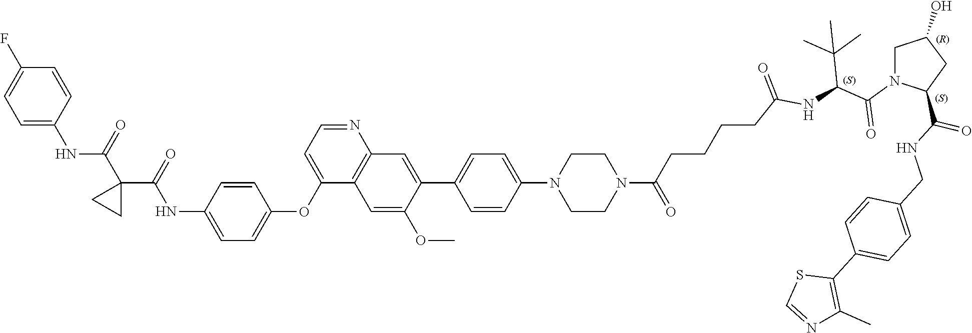

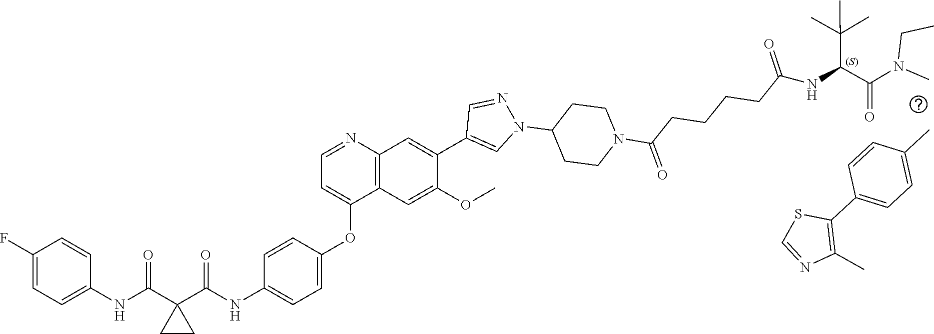

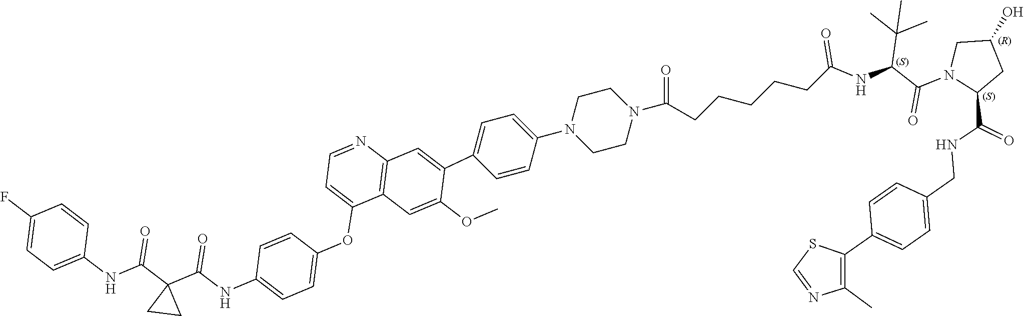

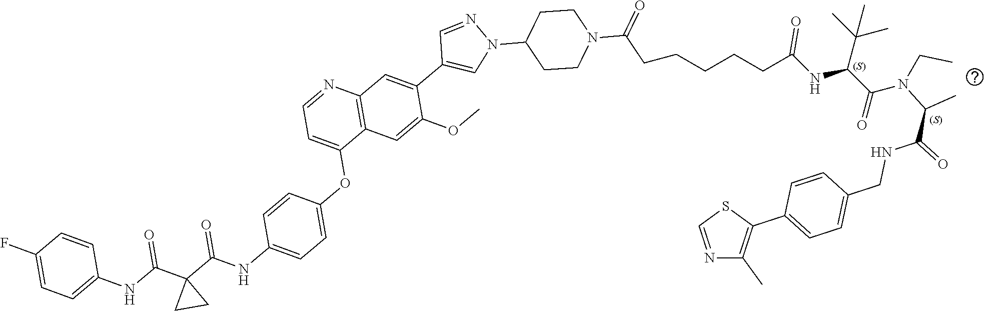

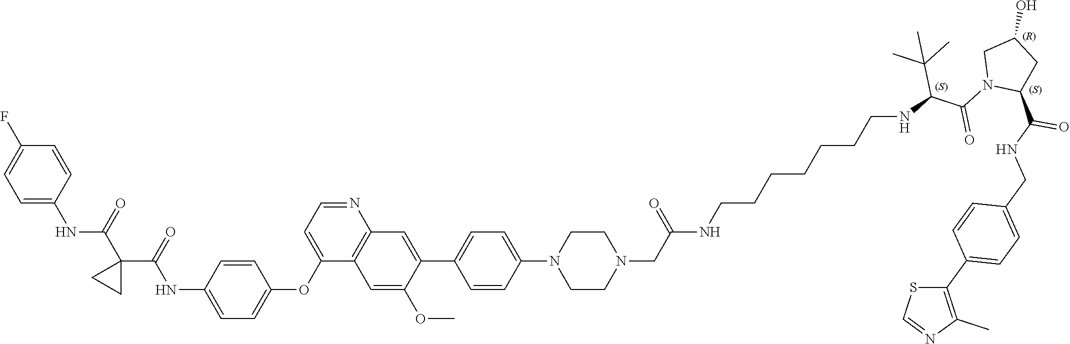

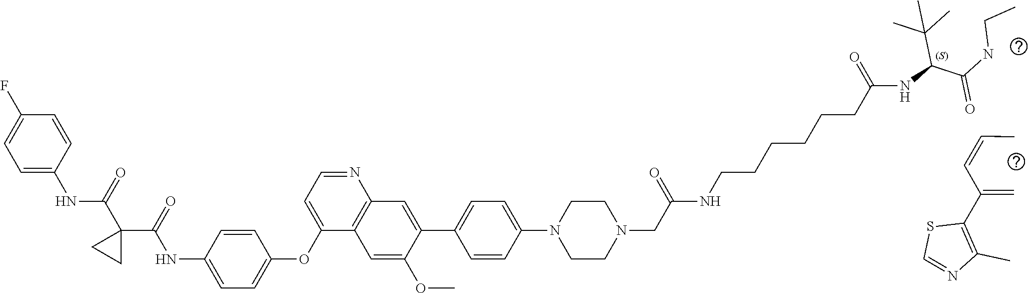

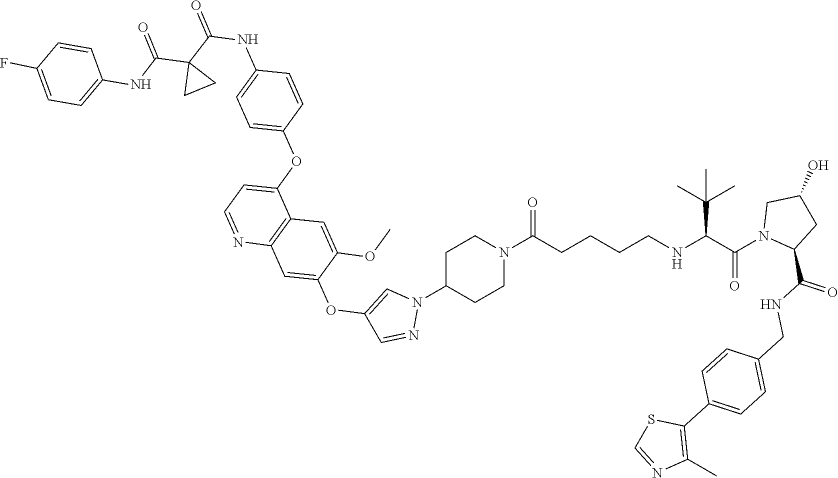 [SMILES: COc1cc2c(Oc3ccc(NC(=O)C4(C(=O)Nc5ccc(F)cc5)CC4)cc3)ccnc2cc1Oc1cnn(C2CCN(C(=O)CCCCN[C@H](C(=O)N3C[C@H](O)C[C@H]3C(=O)NCc3ccc(-c4scnc4C)cc3)C(C)(C)C)CC2)c1]

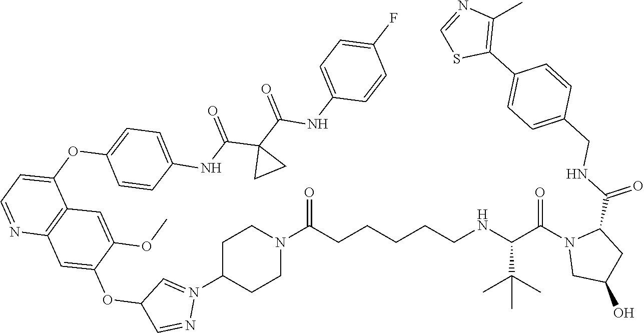

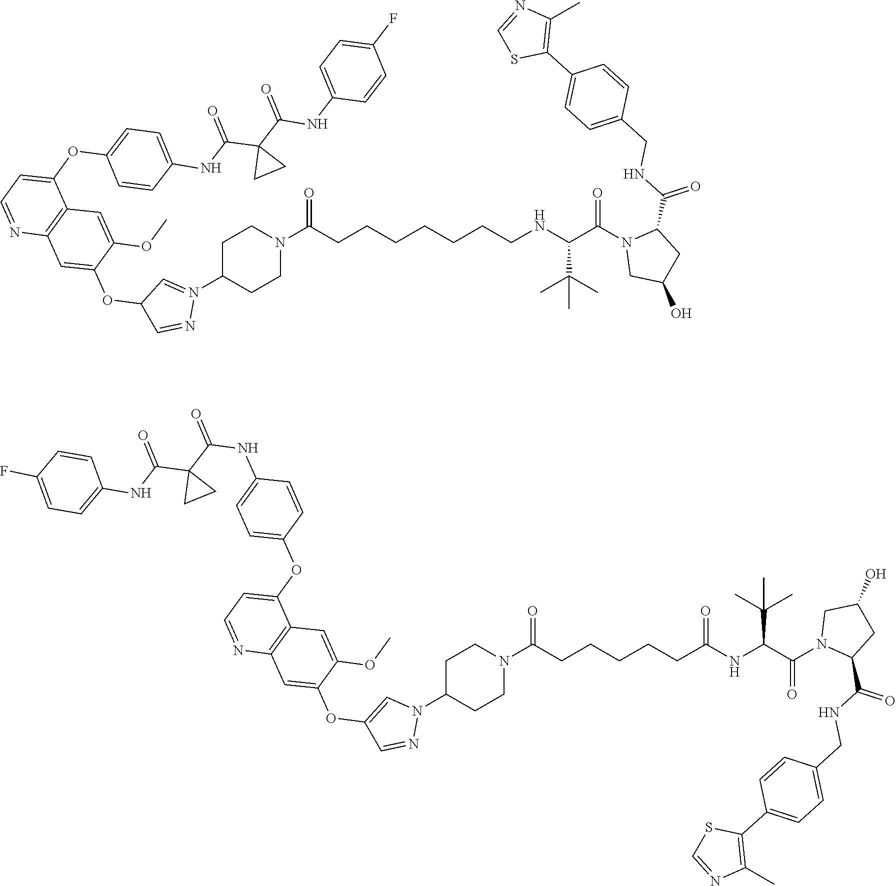

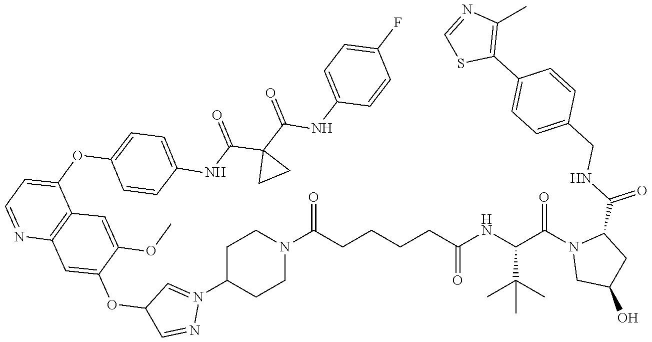

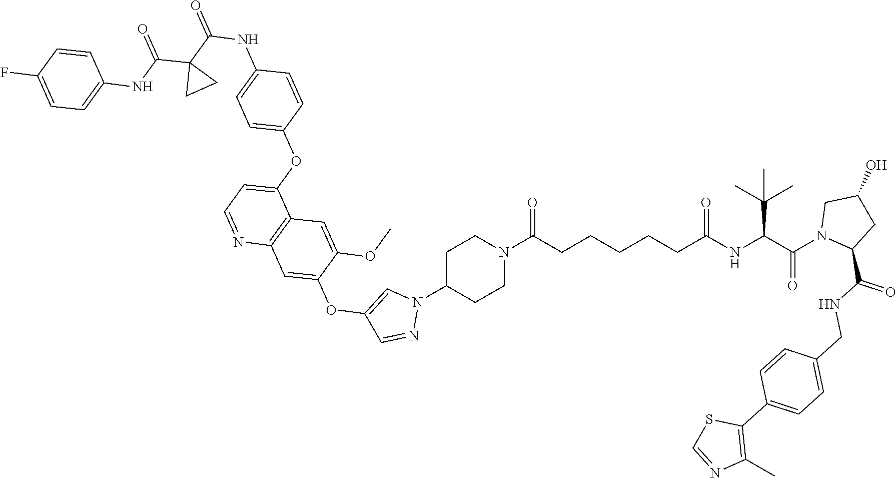 [SMILES: COc1cc2c(Oc3ccc(NC(=O)C4(C(=O)Nc5ccc(F)cc5)CC4)cc3)ccnc2cc1Oc1cnn(C2CCN(C(=O)CCCCCC(=O)N[C@H](C(=O)N3C[C@H](O)C[C@H]3C(=O)NCc3ccc(-c4scnc4C)cc3)C(C)(C)C)CC2)c1]

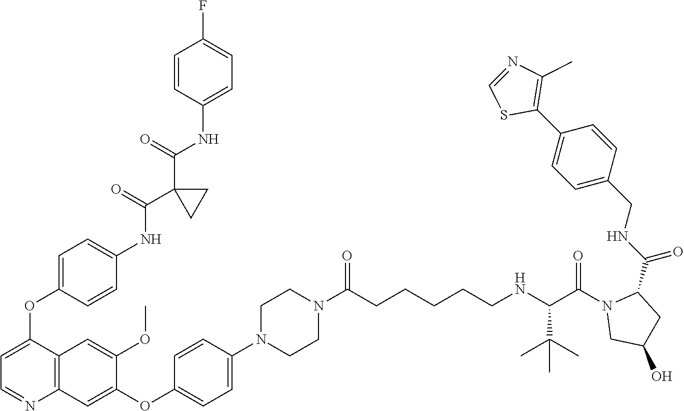 [SMILES: COc1cc2c(Oc3ccc(NC(=O)C4(C(=O)Nc5ccc(F)cc5)CC4)cc3)ccnc2cc1Oc1ccc(N2CCN(C(=O)CCCCCN[C@H](C(=O)N3C[C@H](O)C[C@H]3C(=O)NCc3ccc(-c4scnc4C)cc3)C(C)(C)C)CC2)cc1]

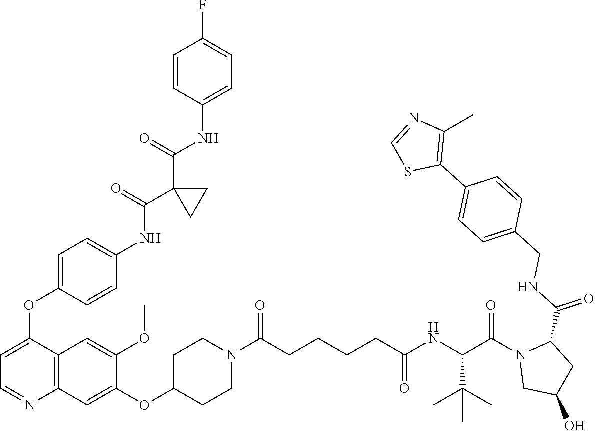 [SMILES: COc1cc2c(Oc3ccc(NC(=O)C4(C(=O)Nc5ccc(F)cc5)CC4)cc3)ccnc2cc1OC1CCN(C(=O)CCCCC(=O)N[C@H](C(=O)N2C[C@H](O)C[C@H]2C(=O)NCc2ccc(-c3scnc3C)cc2)C(C)(C)C)CC1]

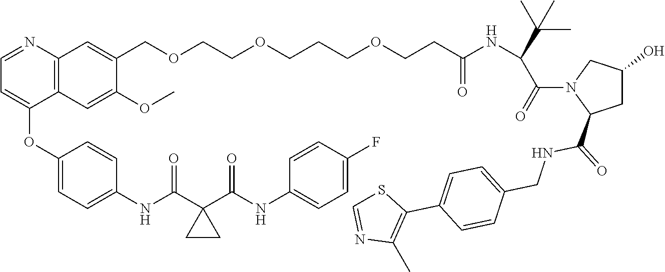 [SMILES: COc1cc2c(Oc3ccc(NC(=O)C4(C(=O)Nc5ccc(F)cc5)CC4)cc3)ccnc2cc1COCCOCCCOCCC(=O)N[C@H](C(=O)N1C[C@H](O)C[C@H]1C(=O)NCc1ccc(-c2scnc2C)cc1)C(C)(C)C]

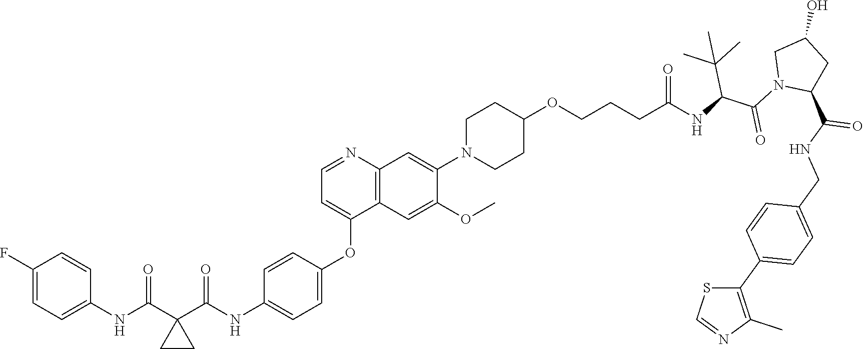 [SMILES: COc1cc2c(Oc3ccc(NC(=O)C4(C(=O)Nc5ccc(F)cc5)CC4)cc3)ccnc2cc1N1CCC(OCCCC(=O)N[C@H](C(=O)N2C[C@H](O)C[C@H]2C(=O)NCc2ccc(-c3scnc3C)cc2)C(C)(C)C)CC1]

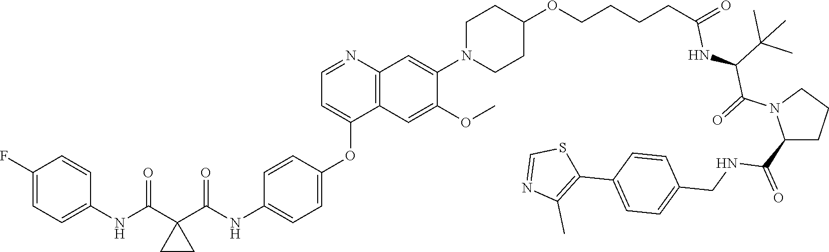 [SMILES: COc1cc2c(Oc3ccc(NC(=O)C4(C(=O)Nc5ccc(F)cc5)CC4)cc3)ccnc2cc1N1CCC(OCCCCC(=O)N[C@H](C(=O)N2CCC[C@H]2C(=O)NCc2ccc(-c3scnc3C)cc2)C(C)(C)C)CC1]

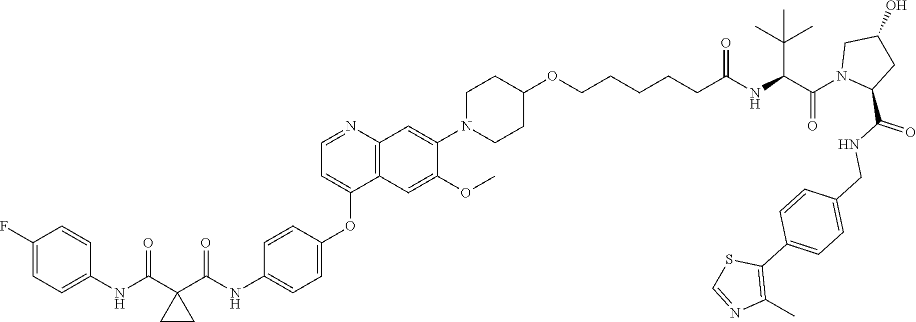 [SMILES: COc1cc2c(Oc3ccc(NC(=O)C4(C(=O)Nc5ccc(F)cc5)CC4)cc3)ccnc2cc1N1CCC(OCCCCCC(=O)N[C@H](C(=O)N2C[C@H](O)C[C@H]2C(=O)NCc2ccc(-c3scnc3C)cc2)C(C)(C)C)CC1]

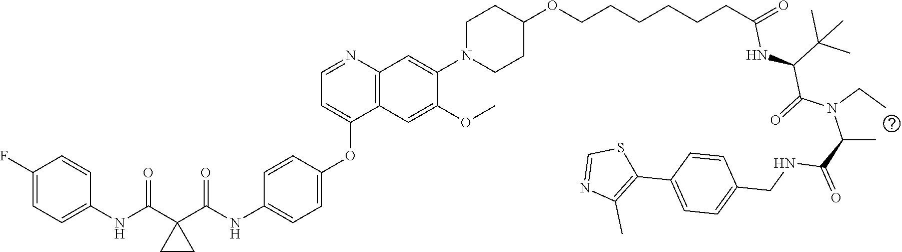 [SMILES: CCN(C(=O)[C@@H](NC(=O)CCCCCCOC1CCN(c2cc3nccc(Oc4ccc(NC(=O)C5(C(=O)Nc6ccc(F)cc6)CC5)cc4)c3cc2OC)CC1)C(C)(C)C)[C@@H](C)C(=O)NCc1ccc(-c2scnc2C)cc1]

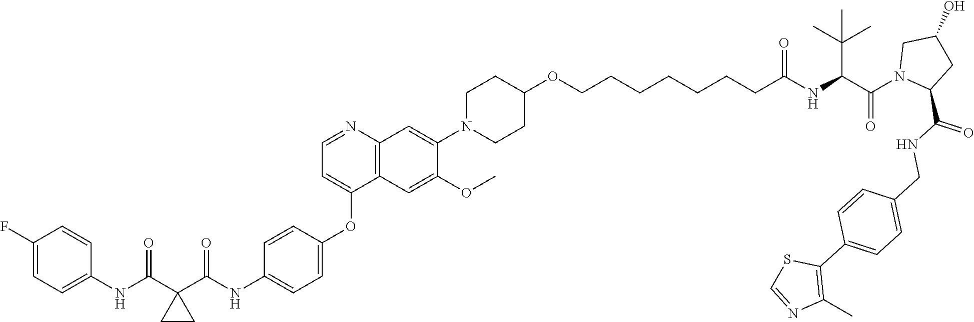 [SMILES: COc1cc2c(Oc3ccc(NC(=O)C4(C(=O)Nc5ccc(F)cc5)CC4)cc3)ccnc2cc1N1CCC(OCCCCCCCC(=O)N[C@H](C(=O)N2C[C@H](O)C[C@H]2C(=O)NCc2ccc(-c3scnc3C)cc2)C(C)(C)C)CC1]

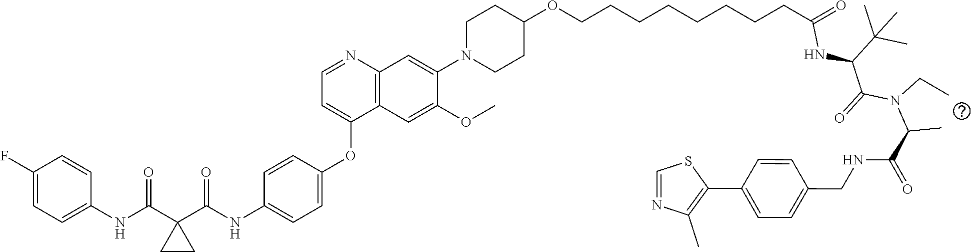 [SMILES: CCN(C(=O)[C@@H](NC(=O)CCCCCCCCOC1CCN(c2cc3nccc(Oc4ccc(NC(=O)C5(C(=O)Nc6ccc(F)cc6)CC5)cc4)c3cc2OC)CC1)C(C)(C)C)[C@@H](C)C(=O)NCc1ccc(-c2scnc2C)cc1]

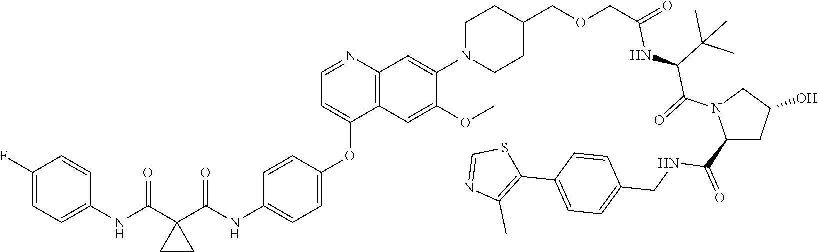 [SMILES: COc1cc2c(Oc3ccc(NC(=O)C4(C(=O)Nc5ccc(F)cc5)CC4)cc3)ccnc2cc1N1CCC(COCC(=O)N[C@H](C(=O)N2C[C@H](O)C[C@H]2C(=O)NCc2ccc(-c3scnc3C)cc2)C(C)(C)C)CC1]

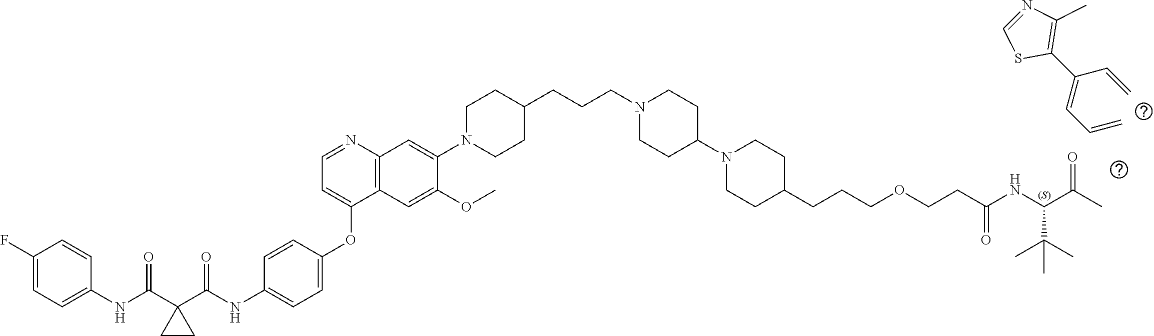

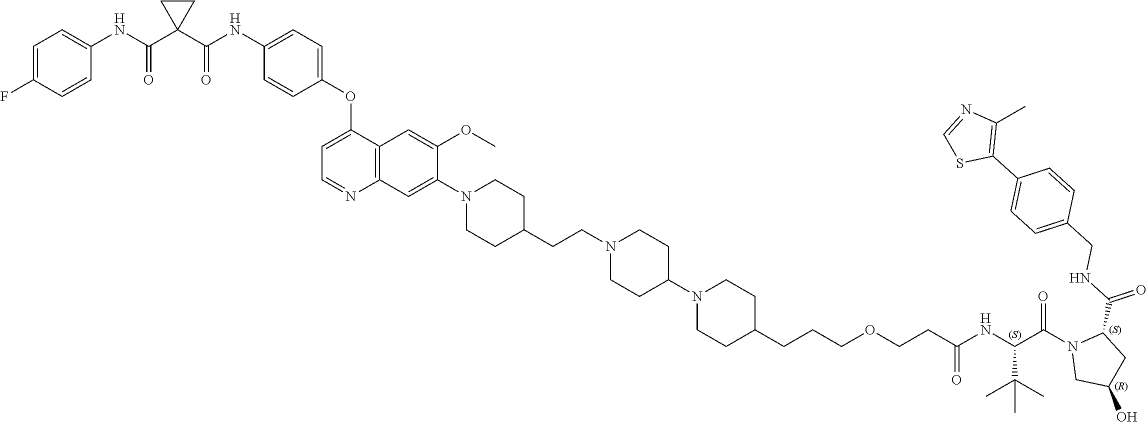

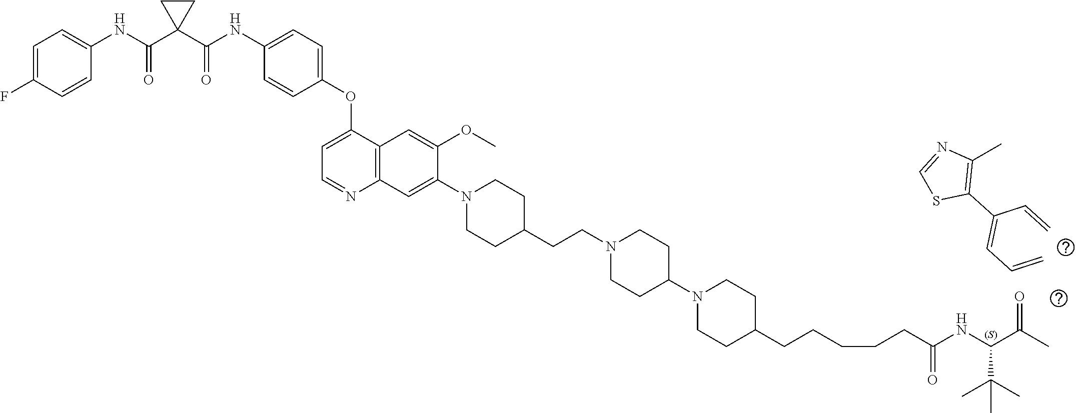

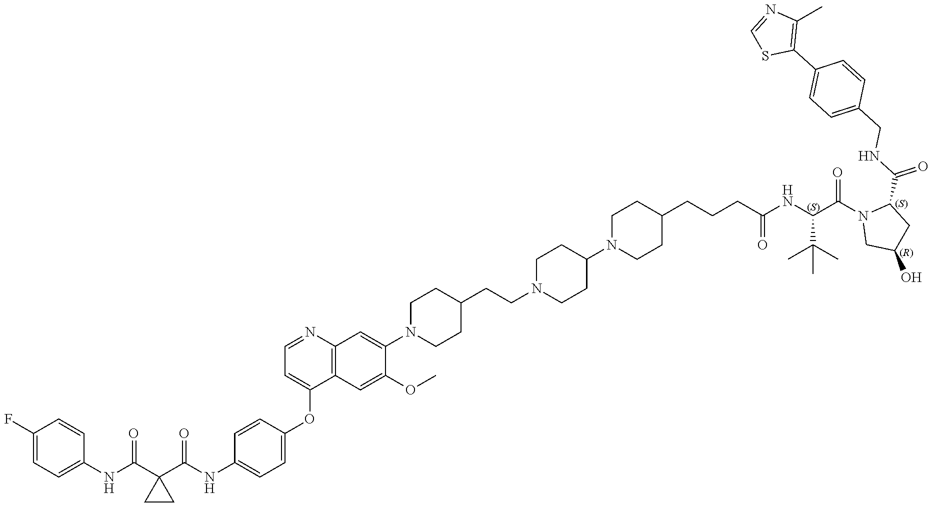

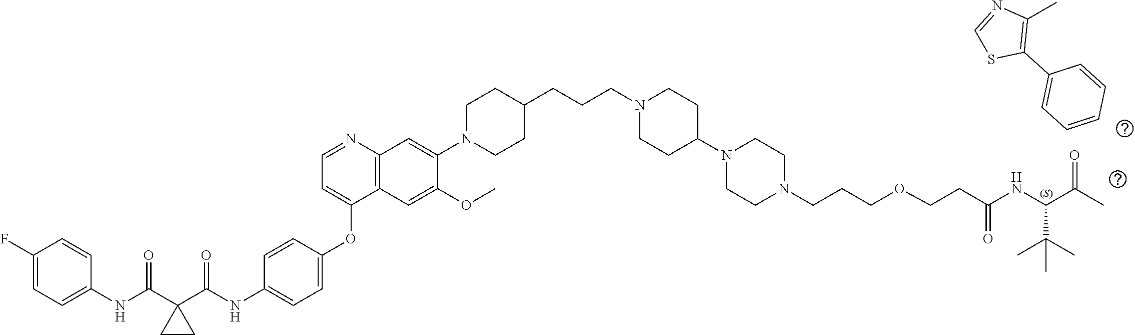

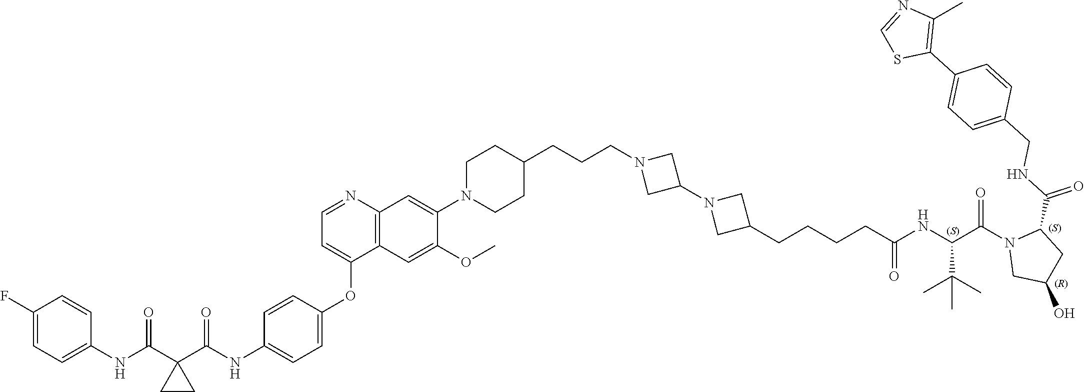

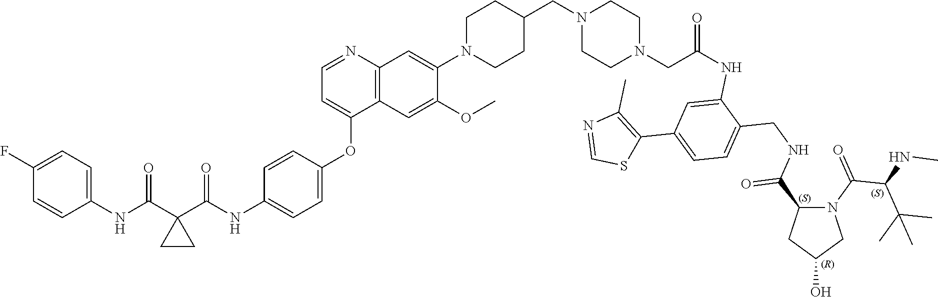

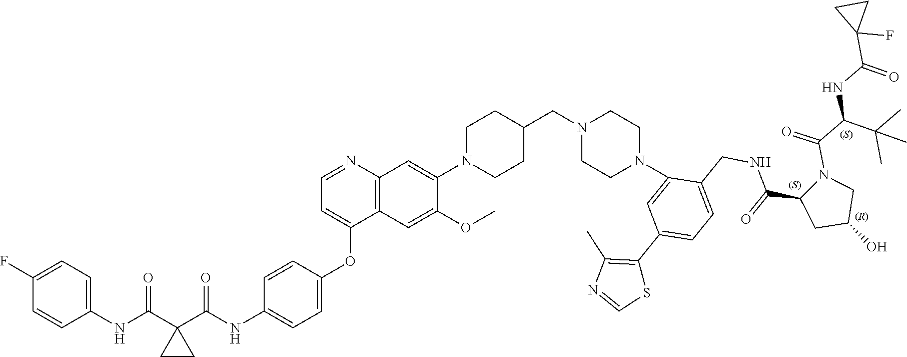

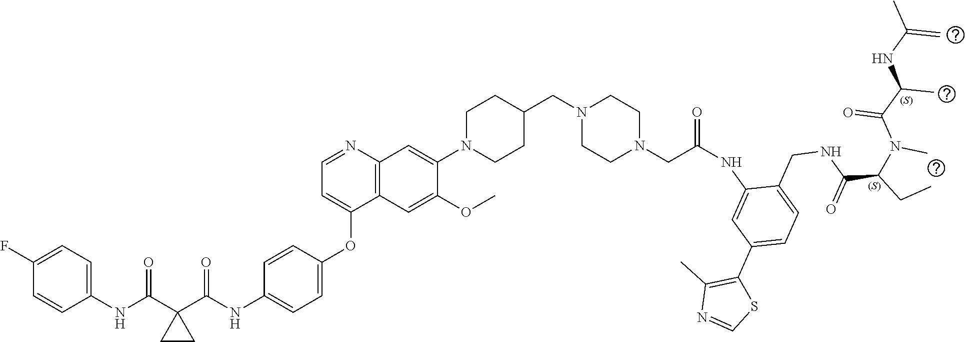

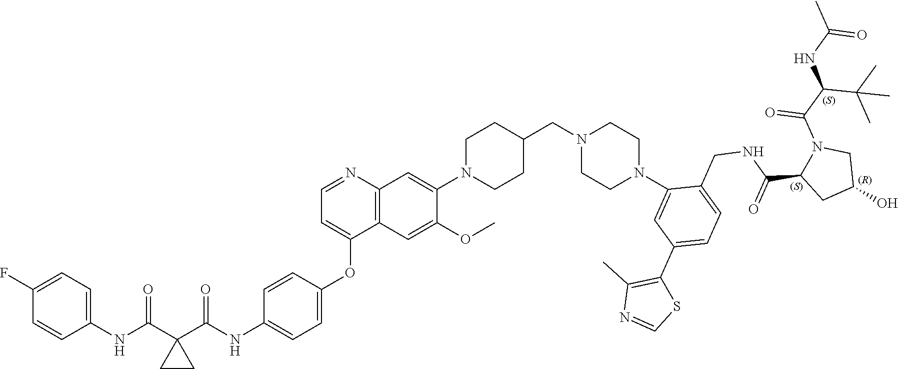

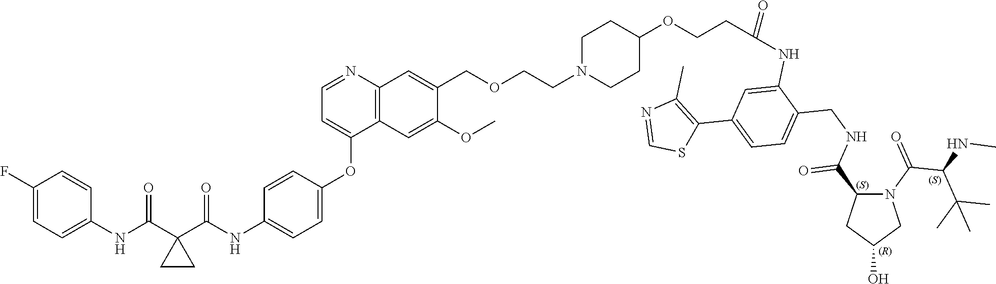

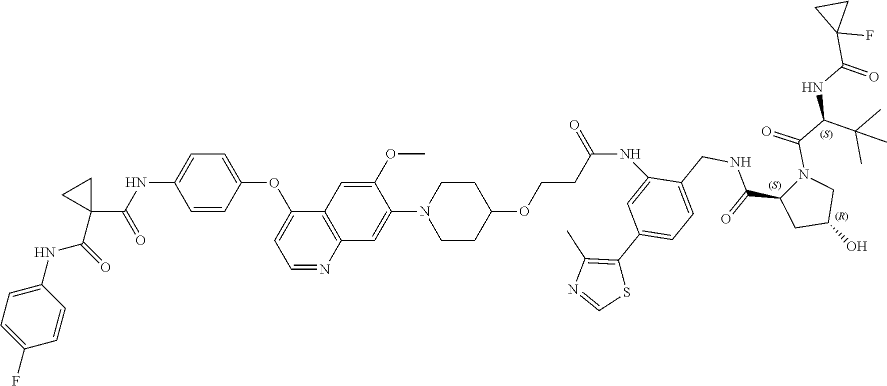

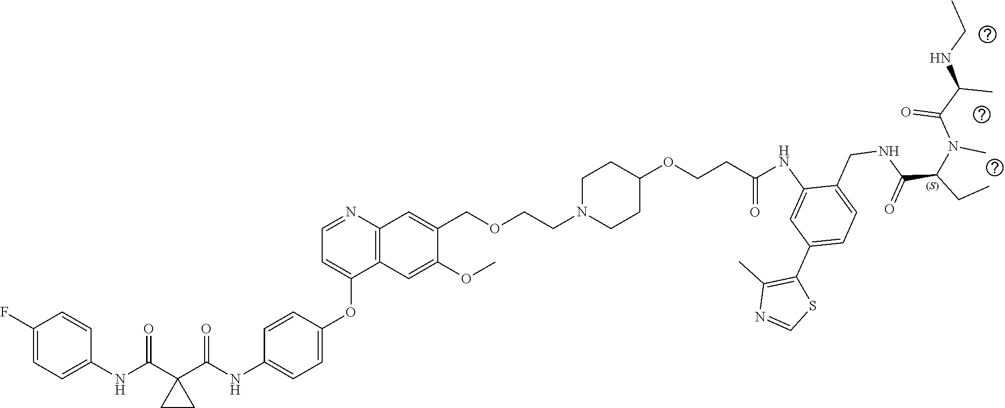

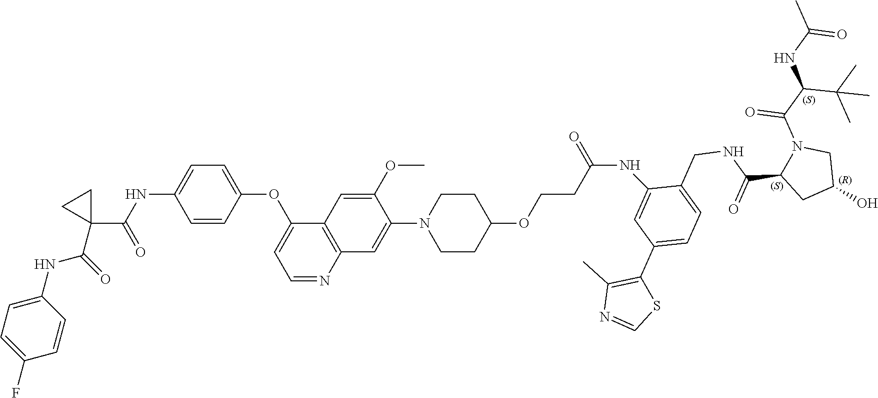

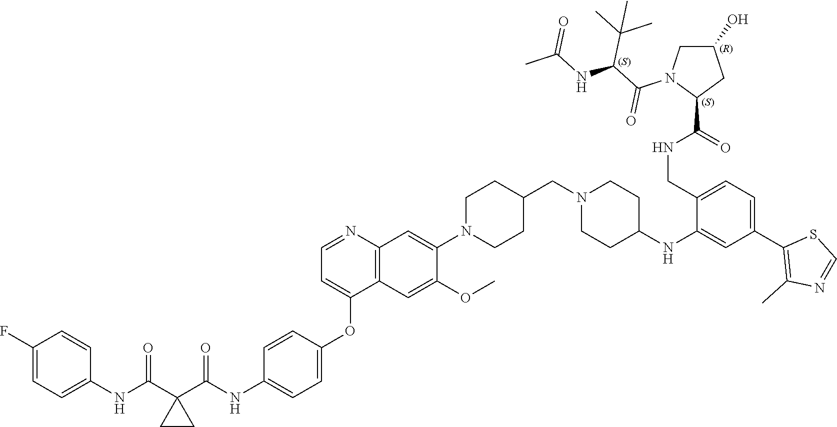

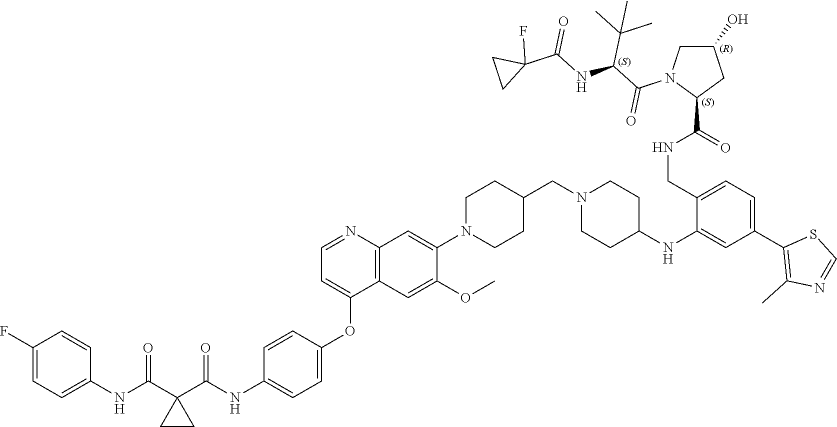

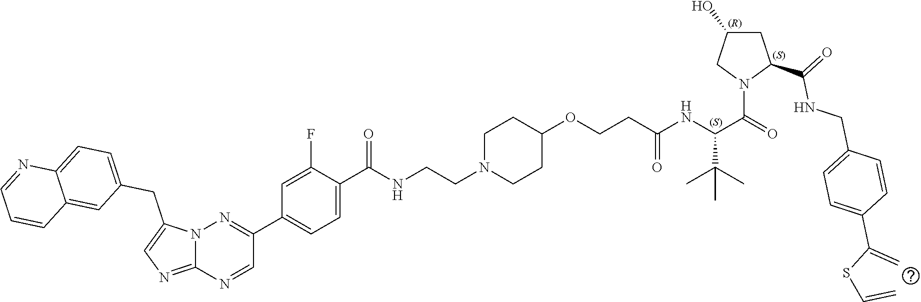

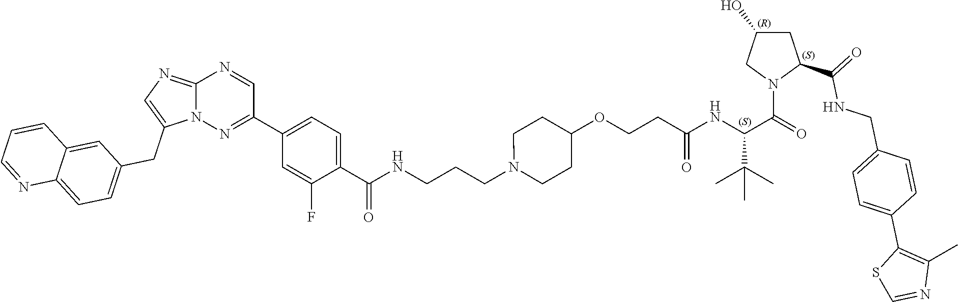

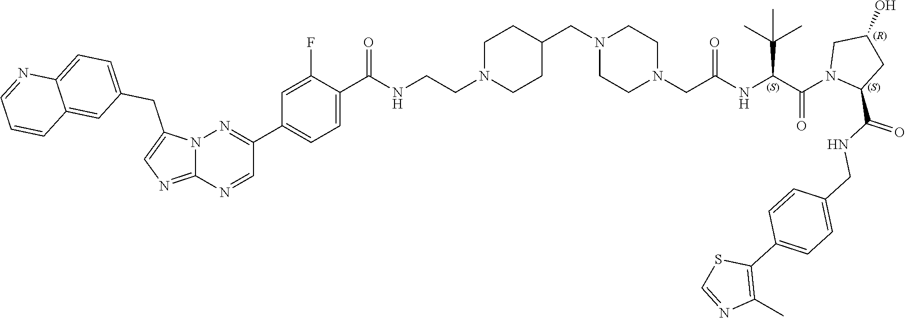

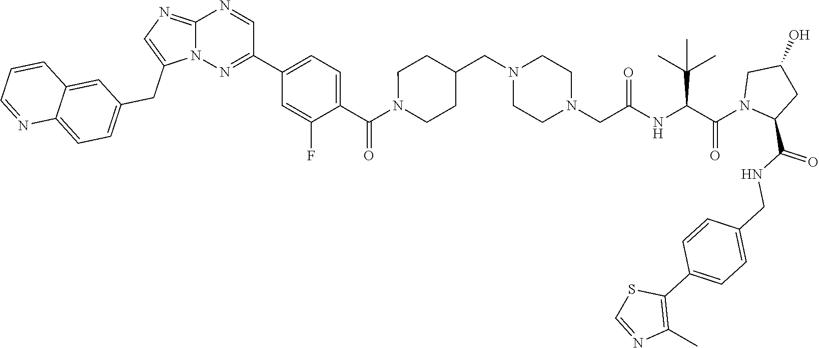 [SMILES: Cc1ncsc1-c1ccc(CNC(=O)[C@@H]2C[C@@H](O)CN2C(=O)[C@@H](NC(=O)CN2CCN(CC3CCN(C(=O)c4ccc(-c5cnc6ncc(Cc7ccc8ncccc8c7)n6n5)cc4F)CC3)CC2)C(C)(C)C)cc1]

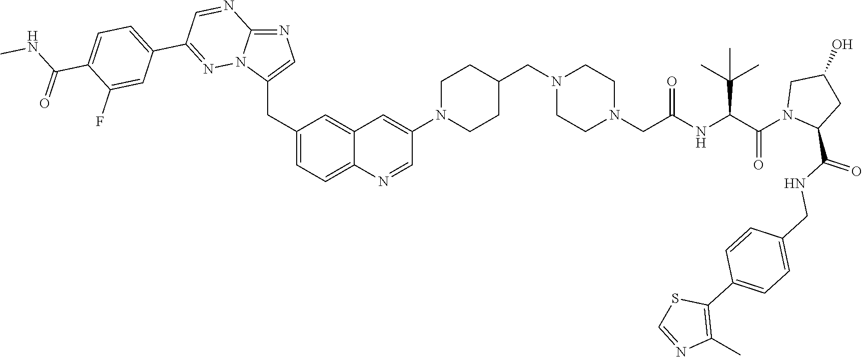 [SMILES: CNC(=O)c1ccc(-c2cnc3ncc(Cc4ccc5ncc(N6CCC(CN7CCN(CC(=O)N[C@H](C(=O)N8C[C@H](O)C[C@H]8C(=O)NCc8ccc(-c9scnc9C)cc8)C(C)(C)C)CC7)CC6)cc5c4)n3n2)cc1F]

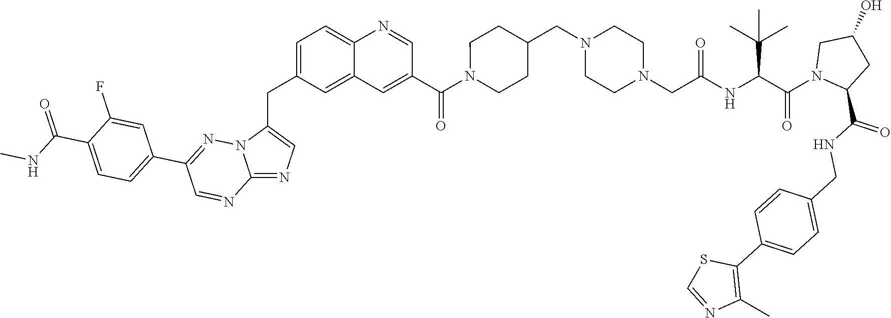 [SMILES: CNC(=O)c1ccc(-c2cnc3ncc(Cc4ccc5ncc(C(=O)N6CCC(CN7CCN(CC(=O)N[C@H](C(=O)N8C[C@H](O)C[C@H]8C(=O)NCc8ccc(-c9scnc9C)cc8)C(C)(C)C)CC7)CC6)cc5c4)n3n2)cc1F]

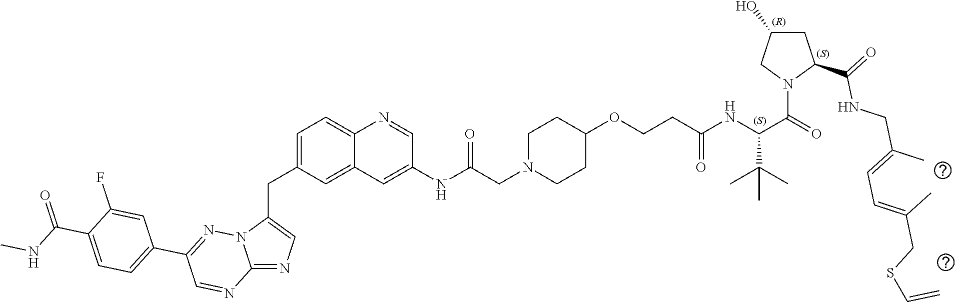

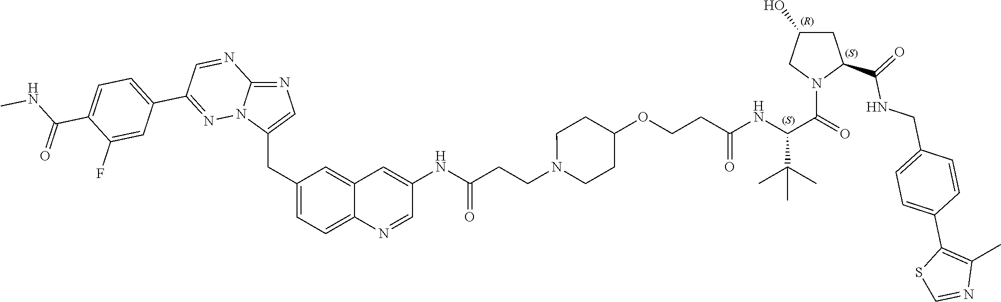

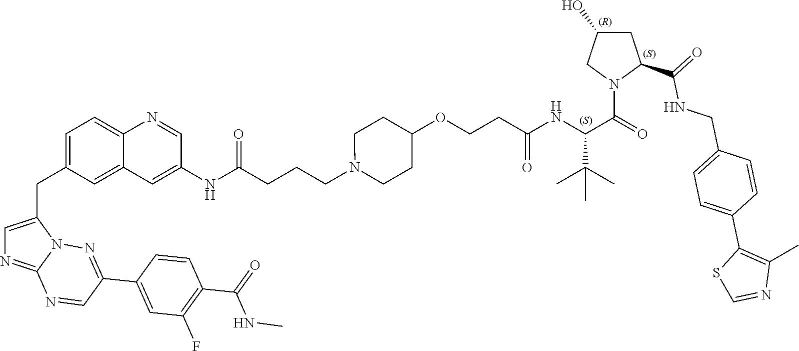

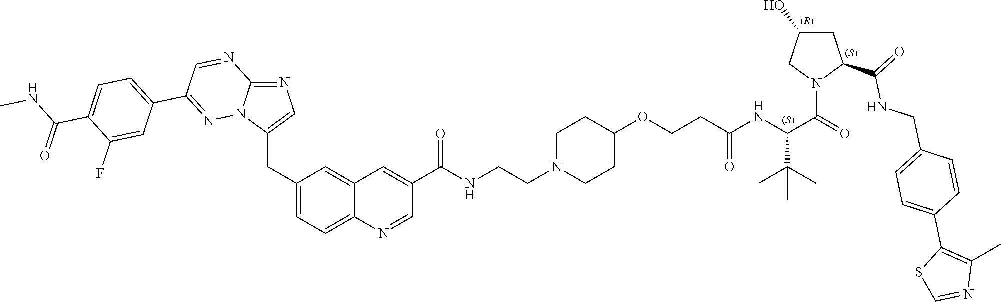

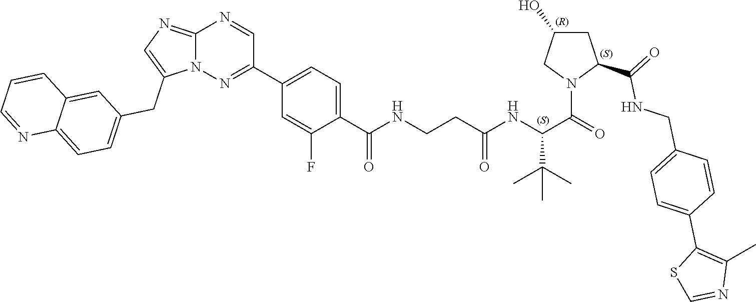

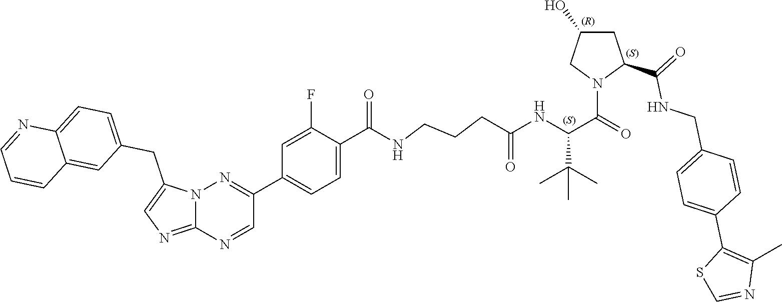

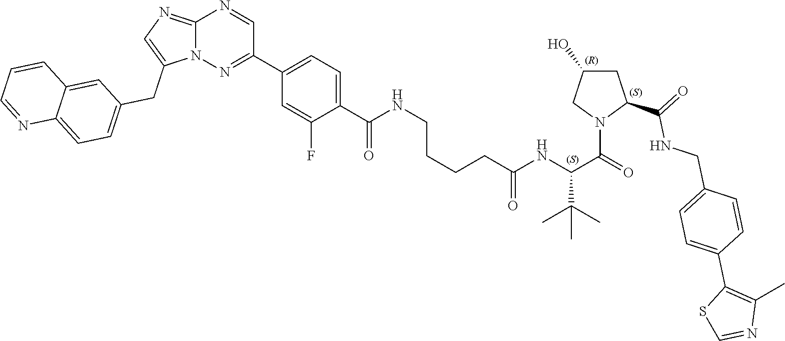

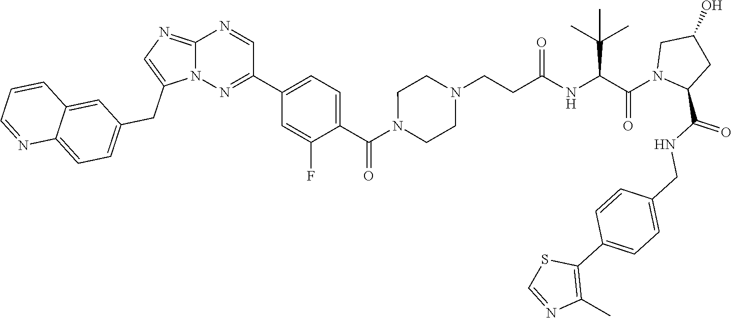 [SMILES: Cc1ncsc1-c1ccc(CNC(=O)[C@@H]2C[C@@H](O)CN2C(=O)[C@@H](NC(=O)CCN2CCN(C(=O)c3ccc(-c4cnc5ncc(Cc6ccc7ncccc7c6)n5n4)cc3F)CC2)C(C)(C)C)cc1]

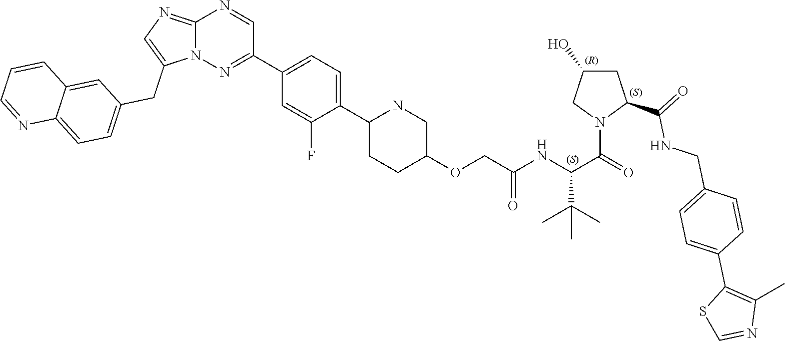

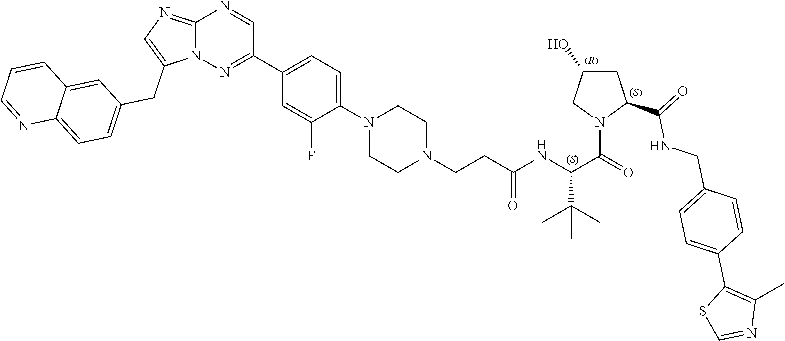

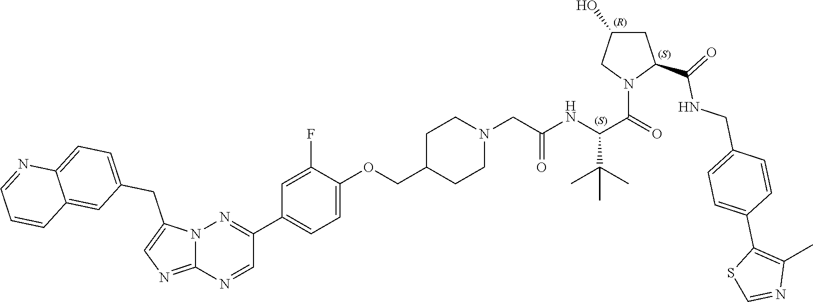

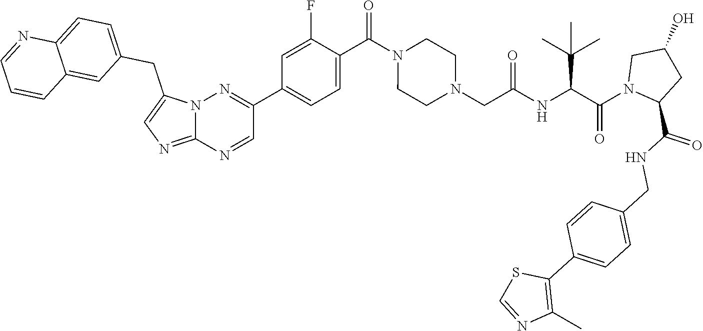 [SMILES: Cc1ncsc1-c1ccc(CNC(=O)[C@@H]2C[C@@H](O)CN2C(=O)[C@@H](NC(=O)CN2CCN(C(=O)c3ccc(-c4cnc5ncc(Cc6ccc7ncccc7c6)n5n4)cc3F)CC2)C(C)(C)C)cc1]

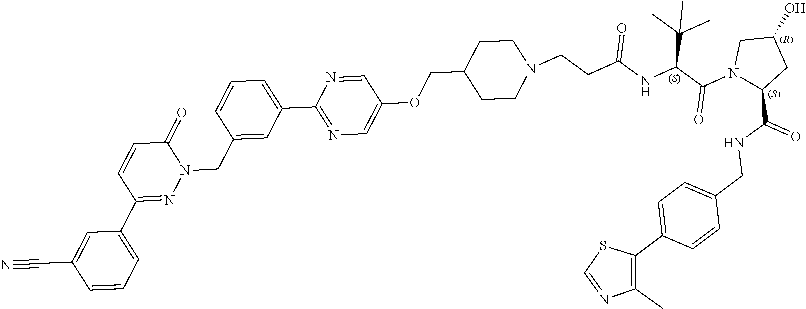

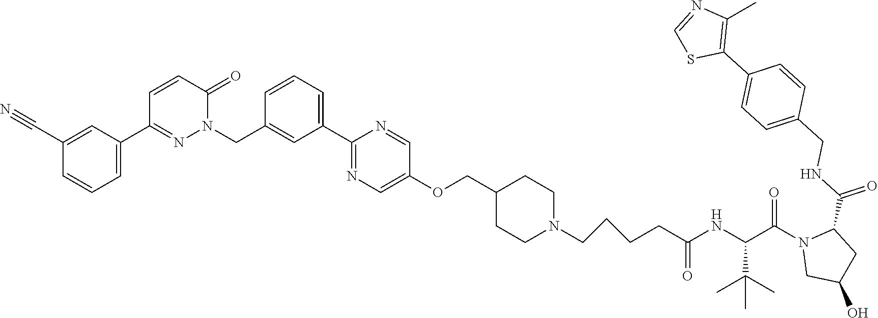 [SMILES: Cc1ncsc1-c1ccc(CNC(=O)[C@@H]2C[C@@H](O)CN2C(=O)[C@@H](NC(=O)CCCCN2CCC(COc3cnc(-c4cccc(Cn5nc(-c6cccc(C#N)c6)ccc5=O)c4)nc3)CC2)C(C)(C)C)cc1]

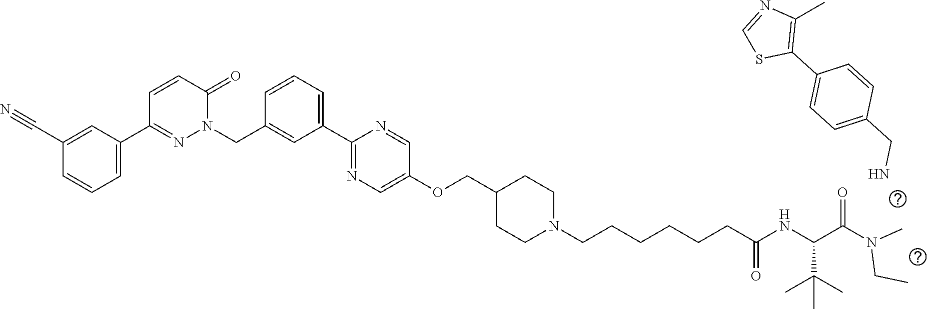

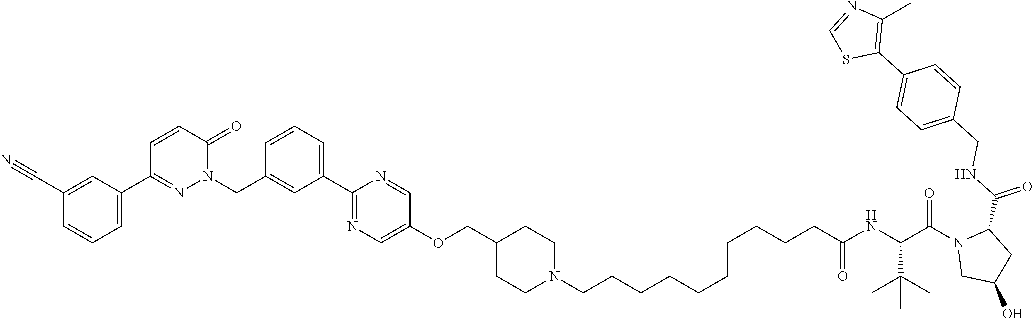 [SMILES: Cc1ncsc1-c1ccc(CNC(=O)[C@@H]2C[C@@H](O)CN2C(=O)[C@@H](NC(=O)CCCCCCCCCCN2CCC(COc3cnc(-c4cccc(Cn5nc(-c6cccc(C#N)c6)ccc5=O)c4)nc3)CC2)C(C)(C)C)cc1]

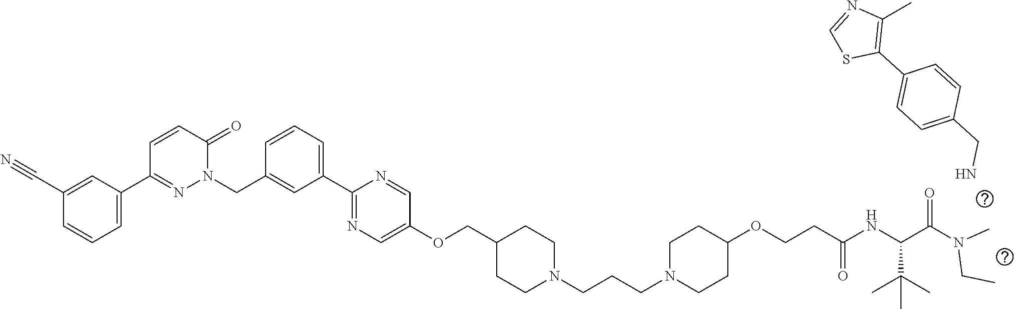

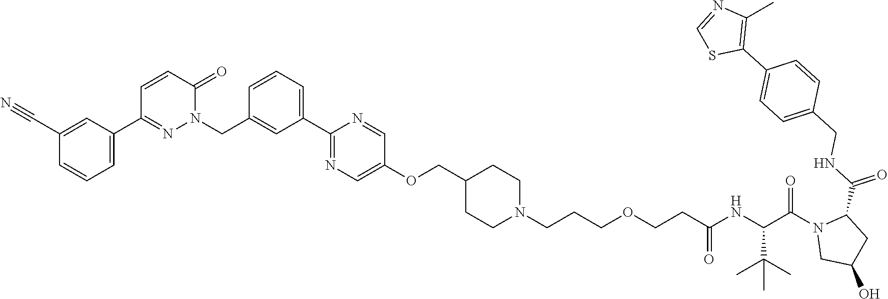 [SMILES: Cc1ncsc1-c1ccc(CNC(=O)[C@@H]2C[C@@H](O)CN2C(=O)[C@@H](NC(=O)CCOCCCN2CCC(COc3cnc(-c4cccc(Cn5nc(-c6cccc(C#N)c6)ccc5=O)c4)nc3)CC2)C(C)(C)C)cc1]

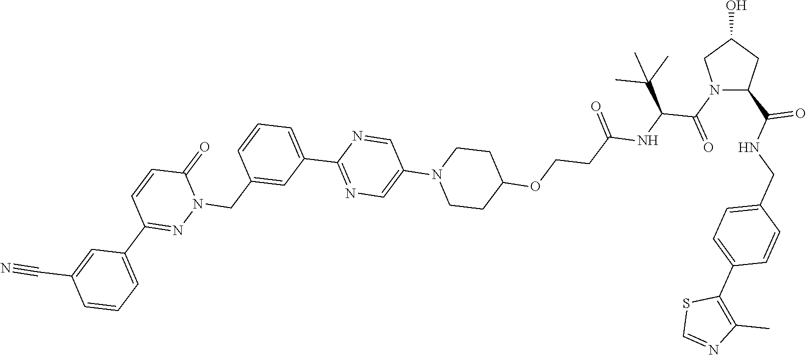 [SMILES: Cc1ncsc1-c1ccc(CNC(=O)[C@@H]2C[C@@H](O)CN2C(=O)[C@@H](NC(=O)CCOC2CCN(c3cnc(-c4cccc(Cn5nc(-c6cccc(C#N)c6)ccc5=O)c4)nc3)CC2)C(C)(C)C)cc1]

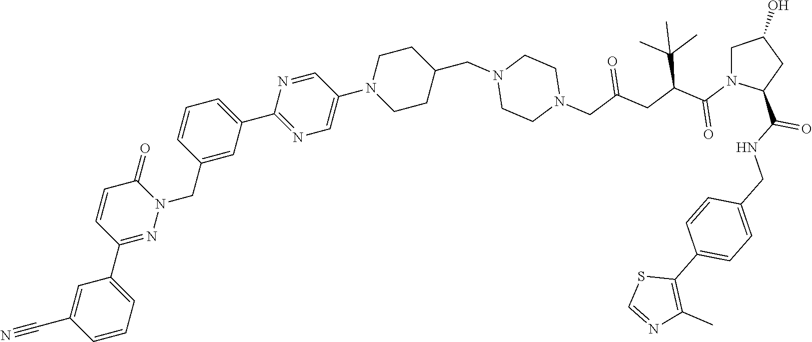 [SMILES: Cc1ncsc1-c1ccc(CNC(=O)[C@@H]2C[C@@H](O)CN2C(=O)[C@@H](CC(=O)CN2CCN(CC3CCN(c4cnc(-c5cccc(Cn6nc(-c7cccc(C#N)c7)ccc6=O)c5)nc4)CC3)CC2)C(C)(C)C)cc1]

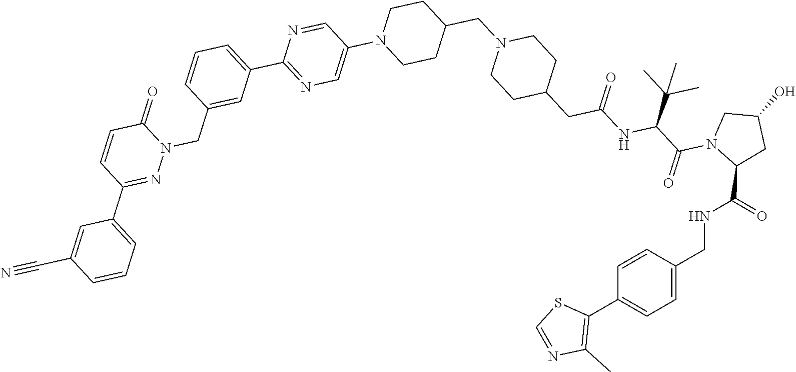 [SMILES: Cc1ncsc1-c1ccc(CNC(=O)[C@@H]2C[C@@H](O)CN2C(=O)[C@@H](NC(=O)CC2CCN(CC3CCN(c4cnc(-c5cccc(Cn6nc(-c7cccc(C#N)c7)ccc6=O)c5)nc4)CC3)CC2)C(C)(C)C)cc1]

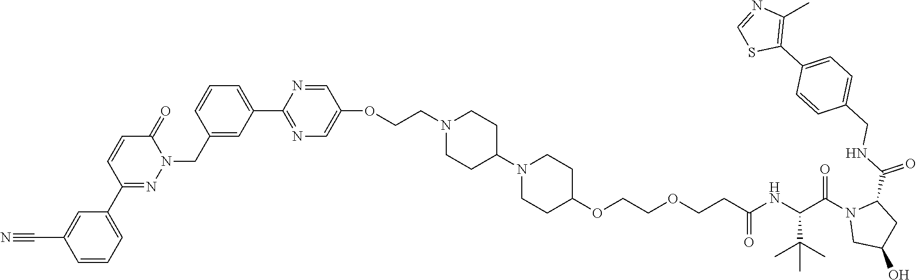 [SMILES: Cc1ncsc1-c1ccc(CNC(=O)[C@@H]2C[C@@H](O)CN2C(=O)[C@@H](NC(=O)CCOCCOC2CCN(C3CCN(CCOc4cnc(-c5cccc(Cn6nc(-c7cccc(C#N)c7)ccc6=O)c5)nc4)CC3)CC2)C(C)(C)C)cc1]

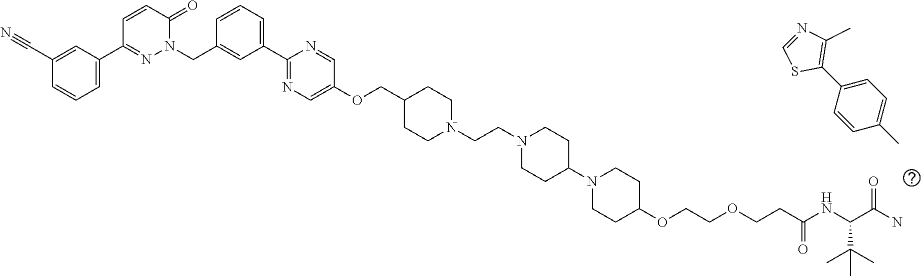 [SMILES: CC(C)(C)[C@H](NC(=O)CCOCCOC1CCN(C2CCN(CCN3CCC(COc4cnc(-c5cccc(Cn6nc(-c7cccc(C#N)c7)ccc6=O)c5)nc4)CC3)CC2)CC1)C(N)=O ; Cc1ccc(-c2scnc2C)cc1]

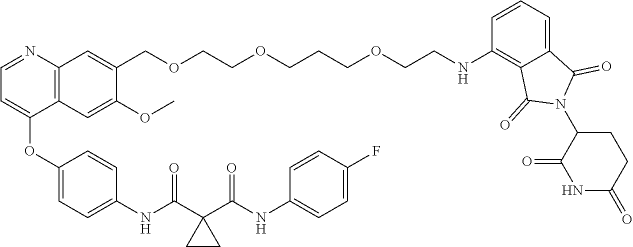 [SMILES: COc1cc2c(Oc3ccc(NC(=O)C4(C(=O)Nc5ccc(F)cc5)CC4)cc3)ccnc2cc1COCCOCCCOCCNc1cccc2c1C(=O)N(C1CCC(=O)NC1=O)C2=O]

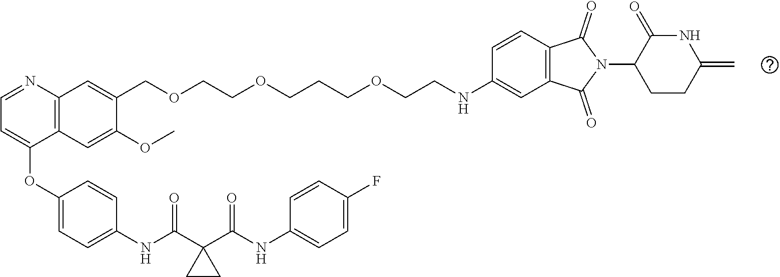 [SMILES: C=C1CCC(N2C(=O)c3ccc(NCCOCCCOCCOCc4cc5nccc(Oc6ccc(NC(=O)C7(C(=O)Nc8ccc(F)cc8)CC7)cc6)c5cc4OC)cc3C2=O)C(=O)N1]

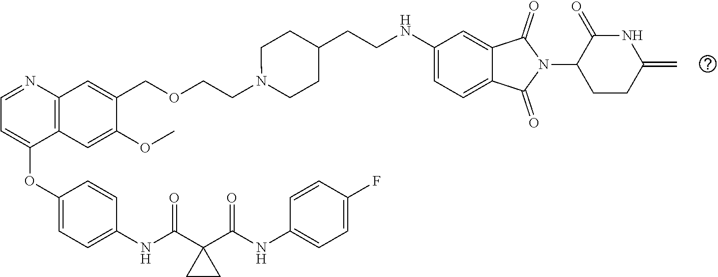 [SMILES: C=C1CCC(N2C(=O)c3ccc(NCCC4CCN(CCOCc5cc6nccc(Oc7ccc(NC(=O)C8(C(=O)Nc9ccc(F)cc9)CC8)cc7)c6cc5OC)CC4)cc3C2=O)C(=O)N1]

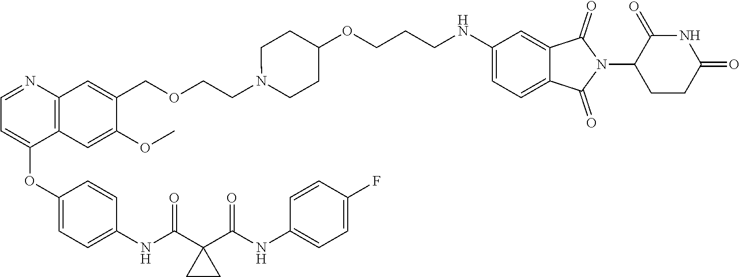 [SMILES: COc1cc2c(Oc3ccc(NC(=O)C4(C(=O)Nc5ccc(F)cc5)CC4)cc3)ccnc2cc1COCCN1CCC(OCCCNc2ccc3c(c2)C(=O)N(C2CCC(=O)NC2=O)C3=O)CC1]

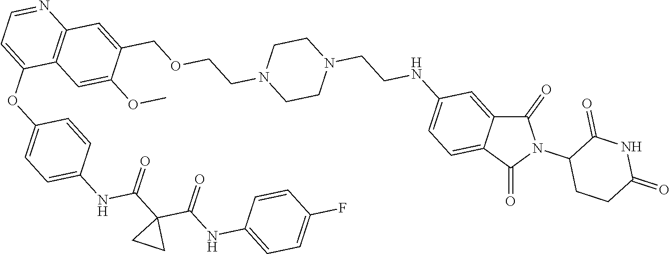 [SMILES: COc1cc2c(Oc3ccc(NC(=O)C4(C(=O)Nc5ccc(F)cc5)CC4)cc3)ccnc2cc1COCCN1CCN(CCNc2ccc3c(c2)C(=O)N(C2CCC(=O)NC2=O)C3=O)CC1]

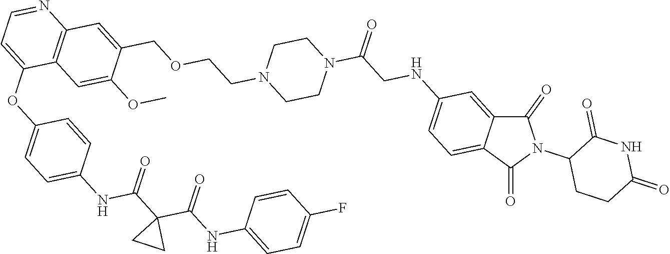 [SMILES: COc1cc2c(Oc3ccc(NC(=O)C4(C(=O)Nc5ccc(F)cc5)CC4)cc3)ccnc2cc1COCCN1CCN(C(=O)CNc2ccc3c(c2)C(=O)N(C2CCC(=O)NC2=O)C3=O)CC1]

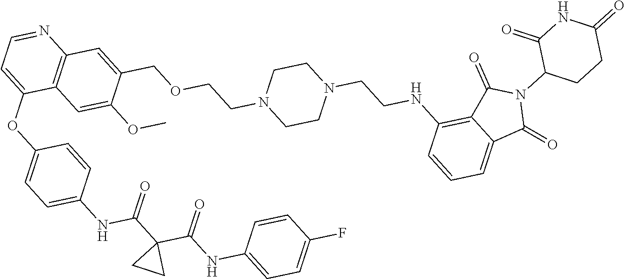 [SMILES: COc1cc2c(Oc3ccc(NC(=O)C4(C(=O)Nc5ccc(F)cc5)CC4)cc3)ccnc2cc1COCCN1CCN(CCNc2cccc3c2C(=O)N(C2CCC(=O)NC2=O)C3=O)CC1]

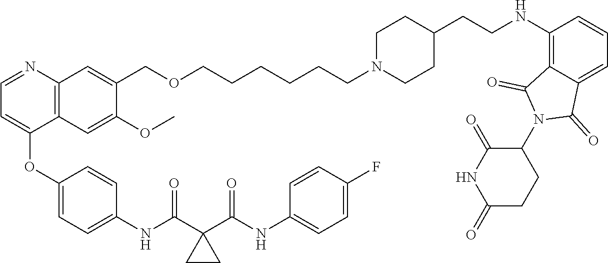 [SMILES: COc1cc2c(Oc3ccc(NC(=O)C4(C(=O)Nc5ccc(F)cc5)CC4)cc3)ccnc2cc1COCCCCCCN1CCC(CCNc2cccc3c2C(=O)N(C2CCC(=O)NC2=O)C3=O)CC1]

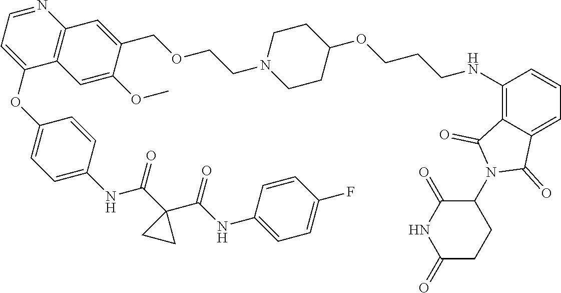 [SMILES: COc1cc2c(Oc3ccc(NC(=O)C4(C(=O)Nc5ccc(F)cc5)CC4)cc3)ccnc2cc1COCCN1CCC(OCCCNc2cccc3c2C(=O)N(C2CCC(=O)NC2=O)C3=O)CC1]

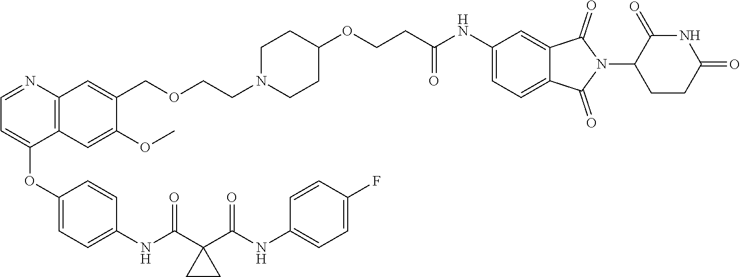 [SMILES: COc1cc2c(Oc3ccc(NC(=O)C4(C(=O)Nc5ccc(F)cc5)CC4)cc3)ccnc2cc1COCCN1CCC(OCCC(=O)Nc2ccc3c(c2)C(=O)N(C2CCC(=O)NC2=O)C3=O)CC1]

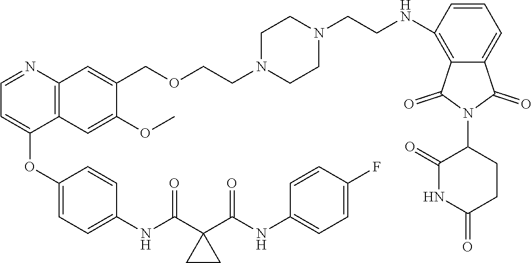 [SMILES: COc1cc2c(Oc3ccc(NC(=O)C4(C(=O)Nc5ccc(F)cc5)CC4)cc3)ccnc2cc1COCCN1CCN(CCNc2cccc3c2C(=O)N(C2CCC(=O)NC2=O)C3=O)CC1]

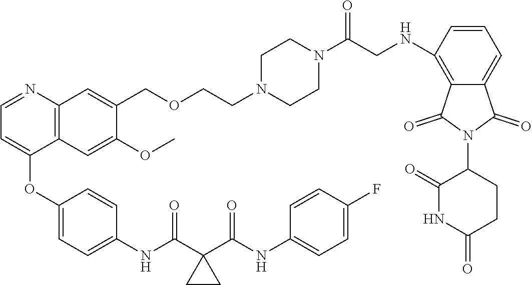 [SMILES: COc1cc2c(Oc3ccc(NC(=O)C4(C(=O)Nc5ccc(F)cc5)CC4)cc3)ccnc2cc1COCCN1CCN(C(=O)CNc2cccc3c2C(=O)N(C2CCC(=O)NC2=O)C3=O)CC1]

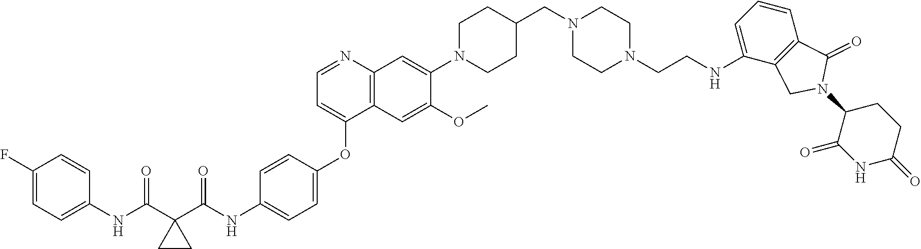 [SMILES: COc1cc2c(Oc3ccc(NC(=O)C4(C(=O)Nc5ccc(F)cc5)CC4)cc3)ccnc2cc1N1CCC(CN2CCN(CCNc3cccc4c3CN([C@H]3CCC(=O)NC3=O)C4=O)CC2)CC1]

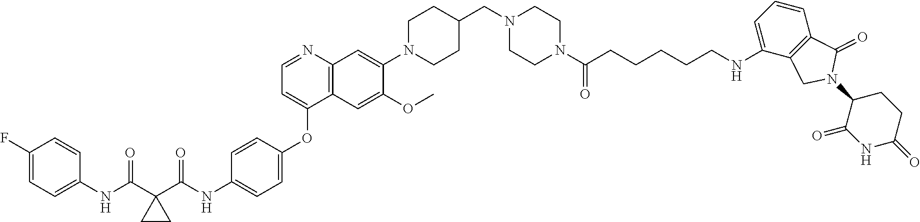 [SMILES: COc1cc2c(Oc3ccc(NC(=O)C4(C(=O)Nc5ccc(F)cc5)CC4)cc3)ccnc2cc1N1CCC(CN2CCN(C(=O)CCCCCNc3cccc4c3CN([C@H]3CCC(=O)NC3=O)C4=O)CC2)CC1]

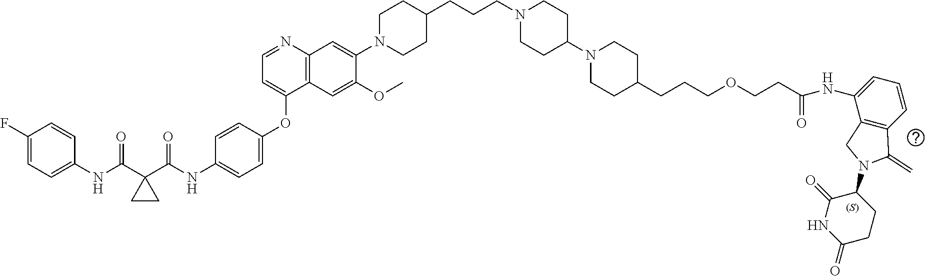

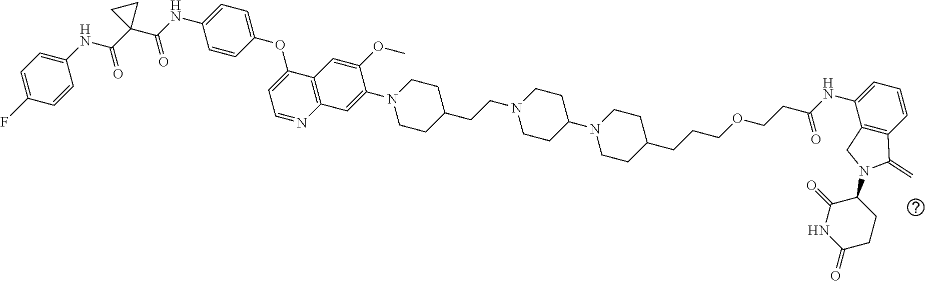 [SMILES: C=C1c2cccc(NC(=O)CCOCCCC3CCN(C4CCN(CCC5CCN(c6cc7nccc(Oc8ccc(NC(=O)C9(C(=O)Nc%10ccc(F)cc%10)CC9)cc8)c7cc6OC)CC5)CC4)CC3)c2CN1[C@H]1CCC(=O)NC1=O]

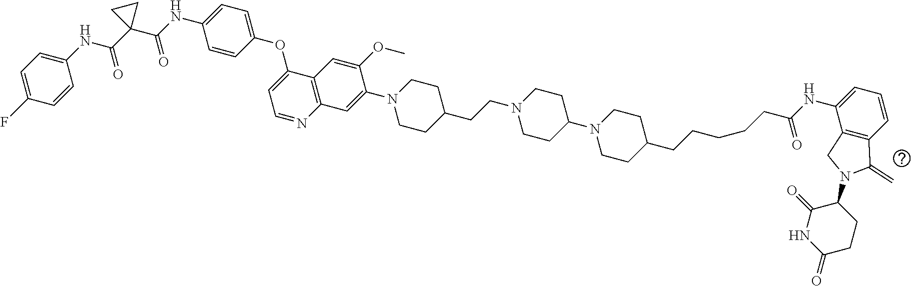 [SMILES: C=C1c2cccc(NC(=O)CCCCCC3CCN(C4CCN(CCC5CCN(c6cc7nccc(Oc8ccc(NC(=O)C9(C(=O)Nc%10ccc(F)cc%10)CC9)cc8)c7cc6OC)CC5)CC4)CC3)c2CN1[C@H]1CCC(=O)NC1=O]

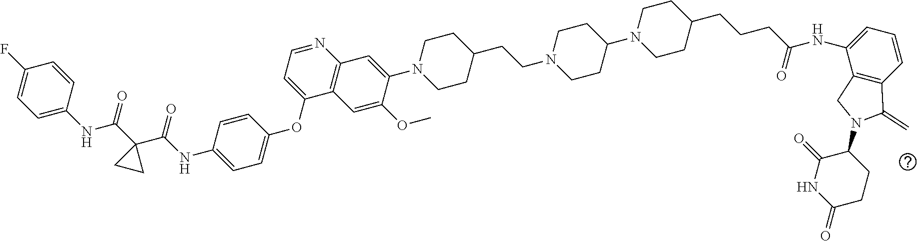 [SMILES: C=C1c2cccc(NC(=O)CCCC3CCN(C4CCN(CCC5CCN(c6cc7nccc(Oc8ccc(NC(=O)C9(C(=O)Nc%10ccc(F)cc%10)CC9)cc8)c7cc6OC)CC5)CC4)CC3)c2CN1[C@H]1CCC(=O)NC1=O]

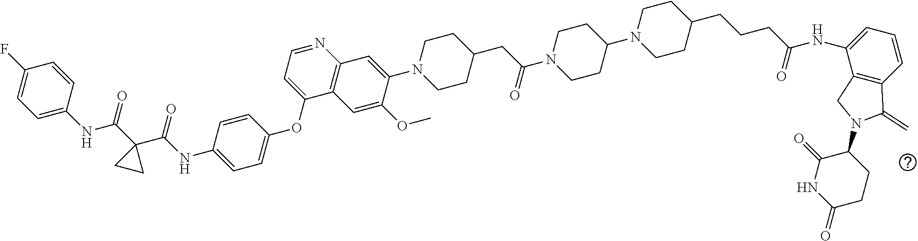 [SMILES: C=C1c2cccc(NC(=O)CCCC3CCN(C4CCN(C(=O)CC5CCN(c6cc7nccc(Oc8ccc(NC(=O)C9(C(=O)Nc%10ccc(F)cc%10)CC9)cc8)c7cc6OC)CC5)CC4)CC3)c2CN1[C@H]1CCC(=O)NC1=O]

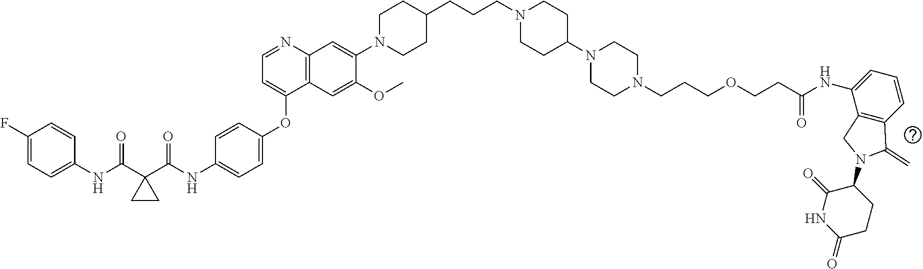 [SMILES: C=C1c2cccc(NC(=O)CCOCCCN3CCN(C4CCN(CCCC5CCN(c6cc7nccc(Oc8ccc(NC(=O)C9(C(=O)Nc%10ccc(F)cc%10)CC9)cc8)c7cc6OC)CC5)CC4)CC3)c2CN1[C@H]1CCC(=O)NC1=O]

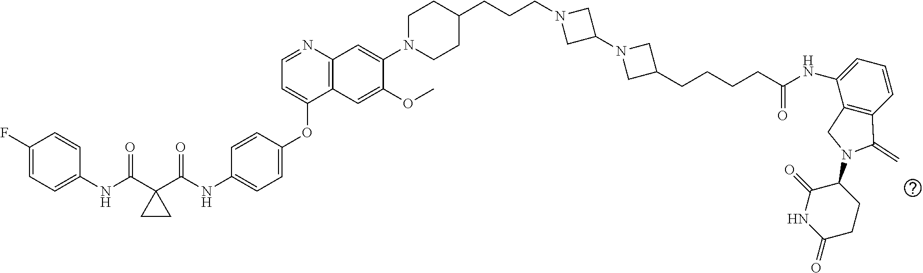 [SMILES: C=C1c2cccc(NC(=O)CCCCC3CN(C4CN(CCCC5CCN(c6cc7nccc(Oc8ccc(NC(=O)C9(C(=O)Nc%10ccc(F)cc%10)CC9)cc8)c7cc6OC)CC5)C4)C3)c2CN1[C@H]1CCC(=O)NC1=O]

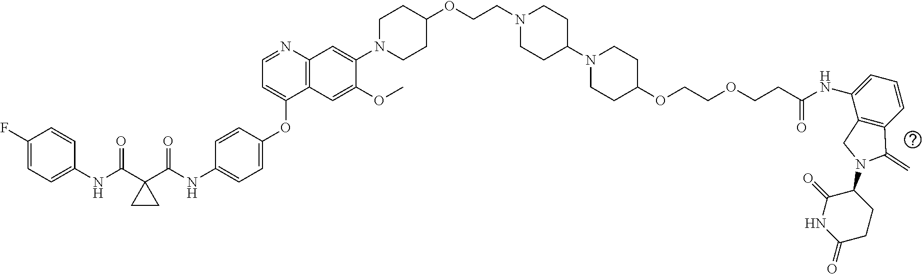 [SMILES: C=C1c2cccc(NC(=O)CCOCCOC3CCN(C4CCN(CCOC5CCN(c6cc7nccc(Oc8ccc(NC(=O)C9(C(=O)Nc%10ccc(F)cc%10)CC9)cc8)c7cc6OC)CC5)CC4)CC3)c2CN1[C@H]1CCC(=O)NC1=O]

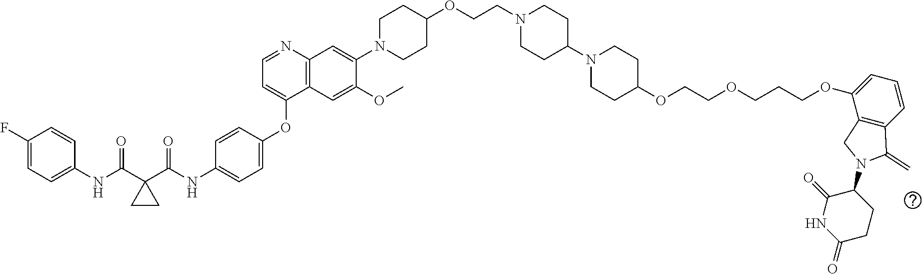 [SMILES: C=C1c2cccc(OCCCOCCOC3CCN(C4CCN(CCOC5CCN(c6cc7nccc(Oc8ccc(NC(=O)C9(C(=O)Nc%10ccc(F)cc%10)CC9)cc8)c7cc6OC)CC5)CC4)CC3)c2CN1[C@H]1CCC(=O)NC1=O]

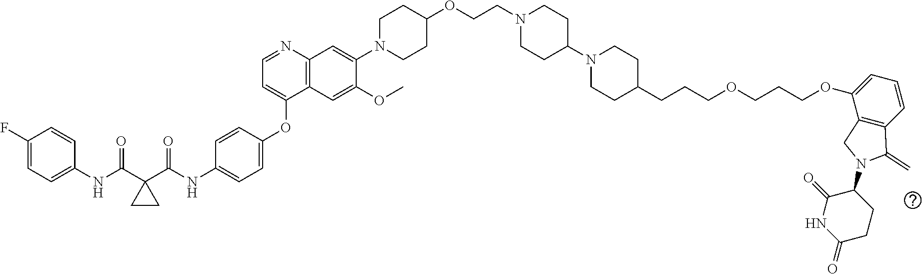 [SMILES: C=C1c2cccc(OCCCOCCCC3CCN(C4CCN(CCOC5CCN(c6cc7nccc(Oc8ccc(NC(=O)C9(C(=O)Nc%10ccc(F)cc%10)CC9)cc8)c7cc6OC)CC5)CC4)CC3)c2CN1[C@H]1CCC(=O)NC1=O]

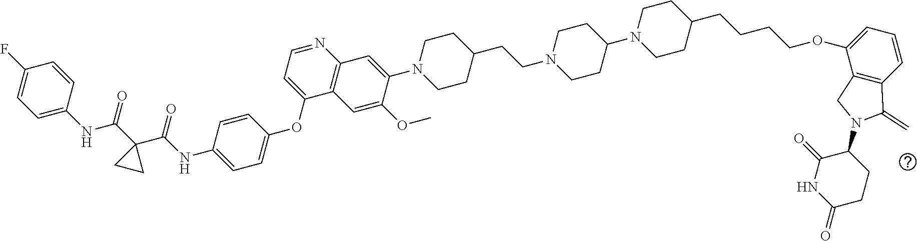 [SMILES: C=C1c2cccc(OCCCCC3CCN(C4CCN(CCC5CCN(c6cc7nccc(Oc8ccc(NC(=O)C9(C(=O)Nc%10ccc(F)cc%10)CC9)cc8)c7cc6OC)CC5)CC4)CC3)c2CN1[C@H]1CCC(=O)NC1=O]

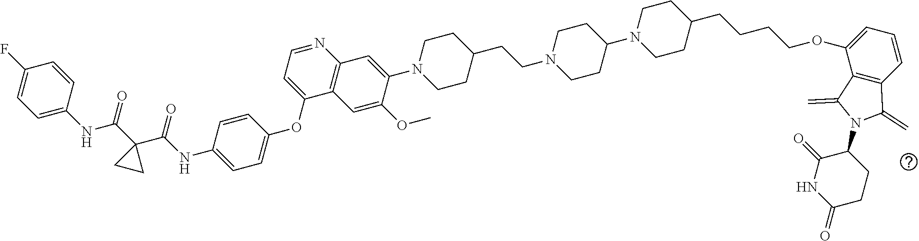 [SMILES: C=c1c2cccc(OCCCCC3CCN(C4CCN(CCC5CCN(c6cc7nccc(Oc8ccc(NC(=O)C9(C(=O)Nc%10ccc(F)cc%10)CC9)cc8)c7cc6OC)CC5)CC4)CC3)c2c(=C)n1[C@H]1CCC(=O)NC1=O]

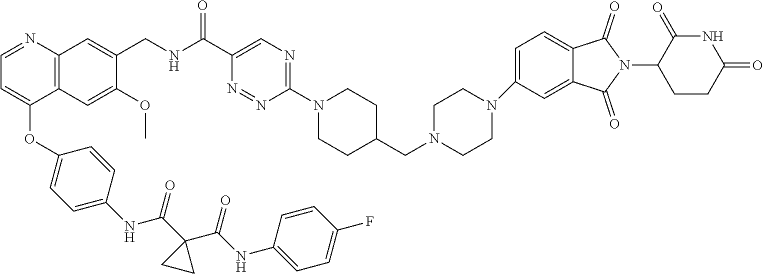 [SMILES: COc1cc2c(Oc3ccc(NC(=O)C4(C(=O)Nc5ccc(F)cc5)CC4)cc3)ccnc2cc1CNC(=O)c1cnc(N2CCC(CN3CCN(c4ccc5c(c4)C(=O)N(C4CCC(=O)NC4=O)C5=O)CC3)CC2)nn1]

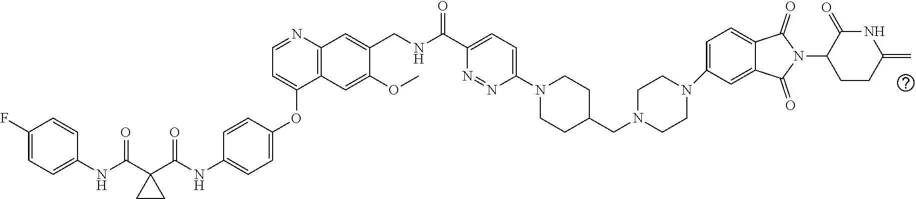 [SMILES: C=C1CCC(N2C(=O)c3ccc(N4CCN(CC5CCN(c6ccc(C(=O)NCc7cc8nccc(Oc9ccc(NC(=O)C%10(C(=O)Nc%11ccc(F)cc%11)CC%10)cc9)c8cc7OC)nn6)CC5)CC4)cc3C2=O)C(=O)N1]

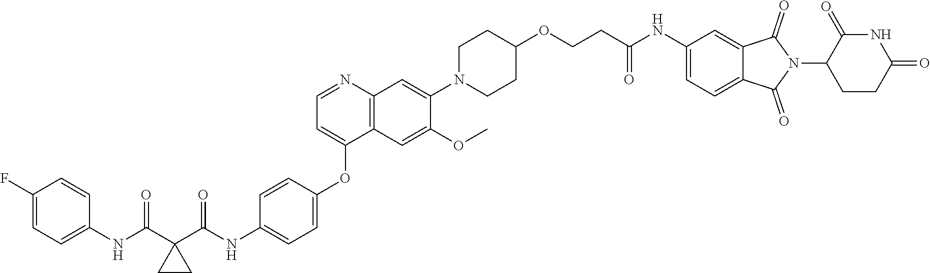 [SMILES: COc1cc2c(Oc3ccc(NC(=O)C4(C(=O)Nc5ccc(F)cc5)CC4)cc3)ccnc2cc1N1CCC(OCCC(=O)Nc2ccc3c(c2)C(=O)N(C2CCC(=O)NC2=O)C3=O)CC1]

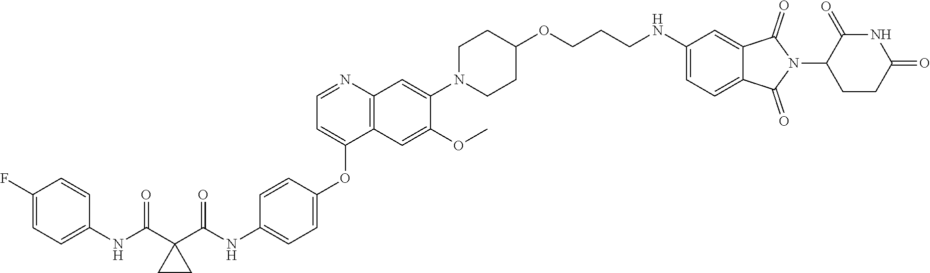 [SMILES: COc1cc2c(Oc3ccc(NC(=O)C4(C(=O)Nc5ccc(F)cc5)CC4)cc3)ccnc2cc1N1CCC(OCCCNc2ccc3c(c2)C(=O)N(C2CCC(=O)NC2=O)C3=O)CC1]

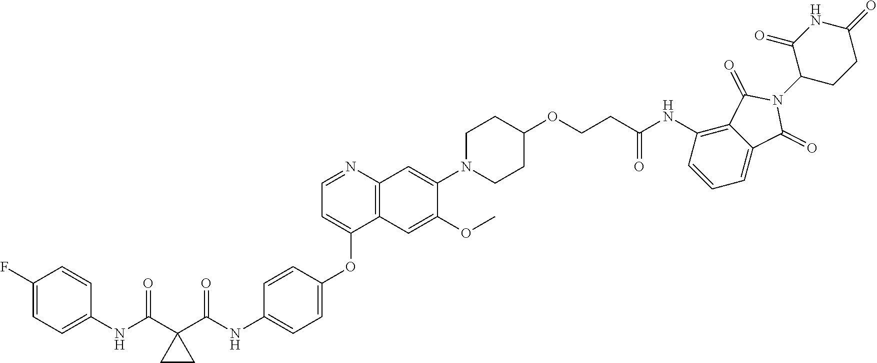 [SMILES: COc1cc2c(Oc3ccc(NC(=O)C4(C(=O)Nc5ccc(F)cc5)CC4)cc3)ccnc2cc1N1CCC(OCCC(=O)Nc2cccc3c2C(=O)N(C2CCC(=O)NC2=O)C3=O)CC1]

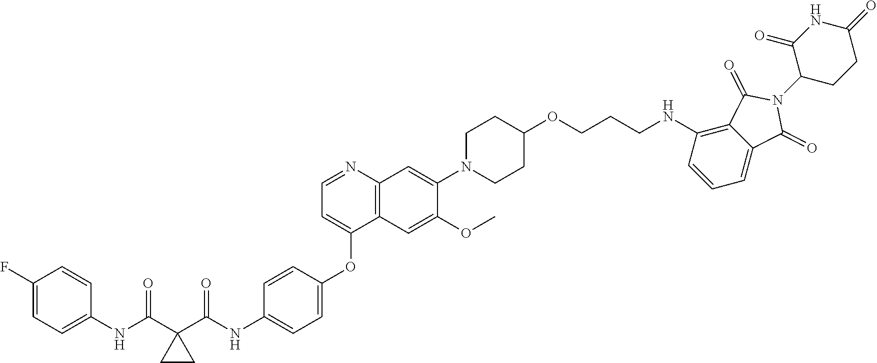 [SMILES: COc1cc2c(Oc3ccc(NC(=O)C4(C(=O)Nc5ccc(F)cc5)CC4)cc3)ccnc2cc1N1CCC(OCCCNc2cccc3c2C(=O)N(C2CCC(=O)NC2=O)C3=O)CC1]

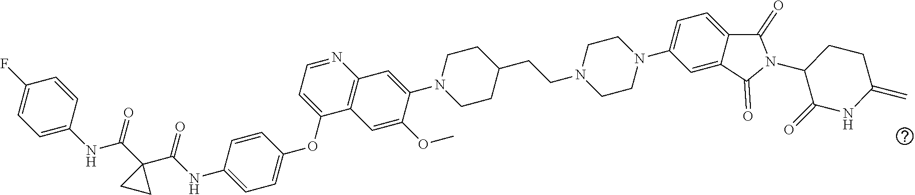 [SMILES: C=C1CCC(N2C(=O)c3ccc(N4CCN(CCC5CCN(c6cc7nccc(Oc8ccc(NC(=O)C9(C(=O)Nc%10ccc(F)cc%10)CC9)cc8)c7cc6OC)CC5)CC4)cc3C2=O)C(=O)N1]

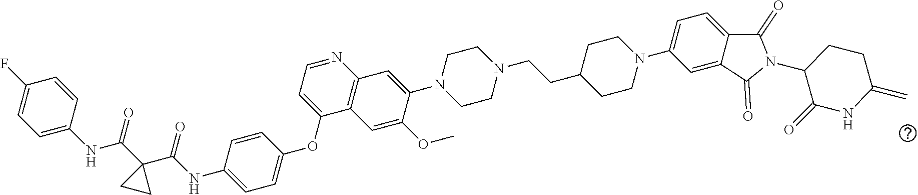 [SMILES: C=C1CCC(N2C(=O)c3ccc(N4CCC(CCN5CCN(c6cc7nccc(Oc8ccc(NC(=O)C9(C(=O)Nc%10ccc(F)cc%10)CC9)cc8)c7cc6OC)CC5)CC4)cc3C2=O)C(=O)N1]

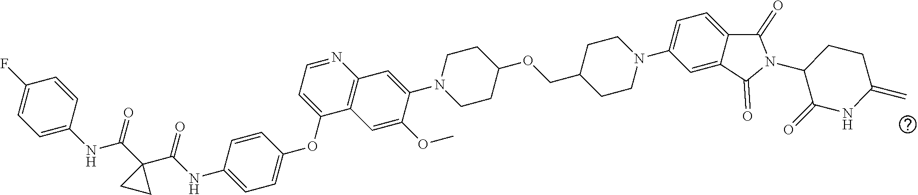 [SMILES: C=C1CCC(N2C(=O)c3ccc(N4CCC(COC5CCN(c6cc7nccc(Oc8ccc(NC(=O)C9(C(=O)Nc%10ccc(F)cc%10)CC9)cc8)c7cc6OC)CC5)CC4)cc3C2=O)C(=O)N1]

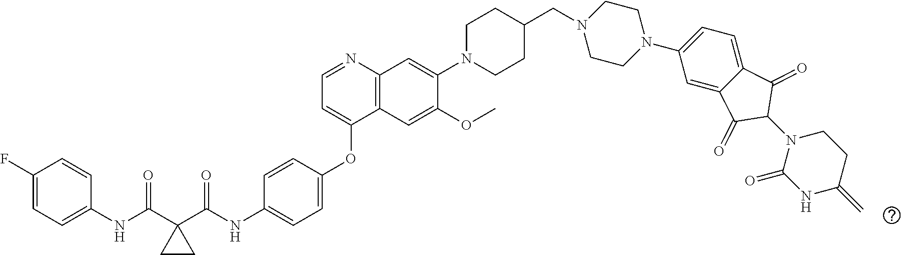 [SMILES: C=C1CCN(C2C(=O)c3ccc(N4CCN(CC5CCN(c6cc7nccc(Oc8ccc(NC(=O)C9(C(=O)Nc%10ccc(F)cc%10)CC9)cc8)c7cc6OC)CC5)CC4)cc3C2=O)C(=O)N1]

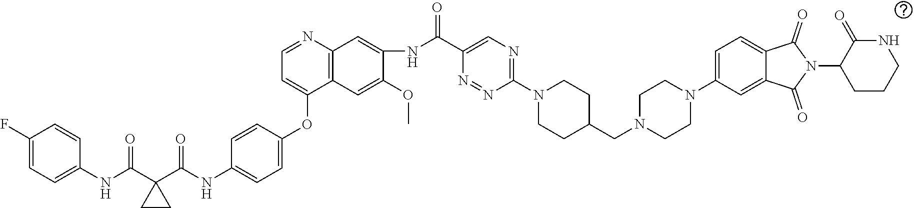 [SMILES: COc1cc2c(Oc3ccc(NC(=O)C4(C(=O)Nc5ccc(F)cc5)CC4)cc3)ccnc2cc1NC(=O)c1cnc(N2CCC(CN3CCN(c4ccc5c(c4)C(=O)N(C4CCCNC4=O)C5=O)CC3)CC2)nn1]

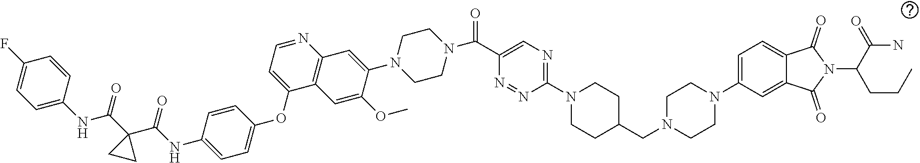 [SMILES: CCCC(C(N)=O)N1C(=O)c2ccc(N3CCN(CC4CCN(c5ncc(C(=O)N6CCN(c7cc8nccc(Oc9ccc(NC(=O)C%10(C(=O)Nc%11ccc(F)cc%11)CC%10)cc9)c8cc7OC)CC6)nn5)CC4)CC3)cc2C1=O]

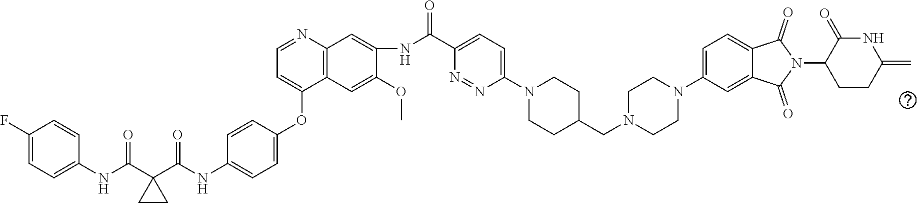 [SMILES: C=C1CCC(N2C(=O)c3ccc(N4CCN(CC5CCN(c6ccc(C(=O)Nc7cc8nccc(Oc9ccc(NC(=O)C%10(C(=O)Nc%11ccc(F)cc%11)CC%10)cc9)c8cc7OC)nn6)CC5)CC4)cc3C2=O)C(=O)N1]

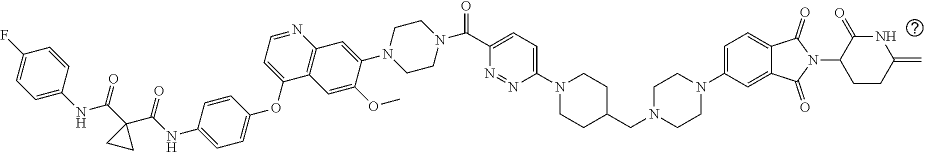 [SMILES: C=C1CCC(N2C(=O)c3ccc(N4CCN(CC5CCN(c6ccc(C(=O)N7CCN(c8cc9nccc(Oc%10ccc(NC(=O)C%11(C(=O)Nc%12ccc(F)cc%12)CC%11)cc%10)c9cc8OC)CC7)nn6)CC5)CC4)cc3C2=O)C(=O)N1]

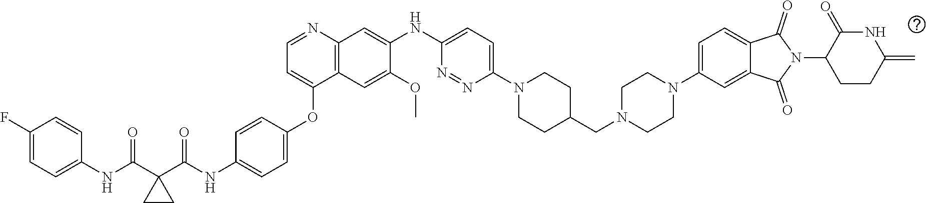 [SMILES: C=C1CCC(N2C(=O)c3ccc(N4CCN(CC5CCN(c6ccc(Nc7cc8nccc(Oc9ccc(NC(=O)C%10(C(=O)Nc%11ccc(F)cc%11)CC%10)cc9)c8cc7OC)nn6)CC5)CC4)cc3C2=O)C(=O)N1]

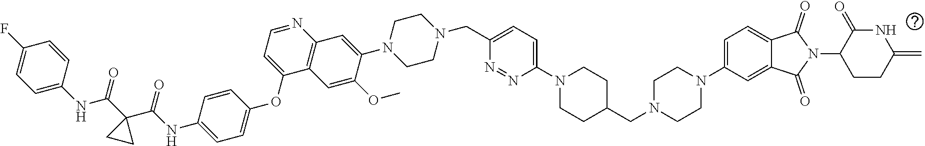 [SMILES: C=C1CCC(N2C(=O)c3ccc(N4CCN(CC5CCN(c6ccc(CN7CCN(c8cc9nccc(Oc%10ccc(NC(=O)C%11(C(=O)Nc%12ccc(F)cc%12)CC%11)cc%10)c9cc8OC)CC7)nn6)CC5)CC4)cc3C2=O)C(=O)N1]

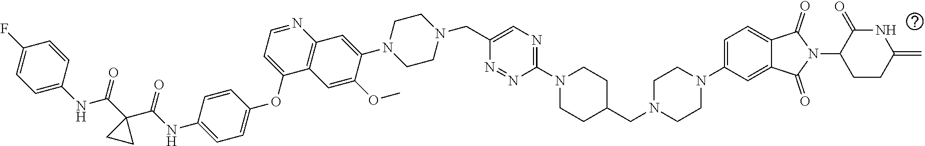 [SMILES: C=C1CCC(N2C(=O)c3ccc(N4CCN(CC5CCN(c6ncc(CN7CCN(c8cc9nccc(Oc%10ccc(NC(=O)C%11(C(=O)Nc%12ccc(F)cc%12)CC%11)cc%10)c9cc8OC)CC7)nn6)CC5)CC4)cc3C2=O)C(=O)N1]

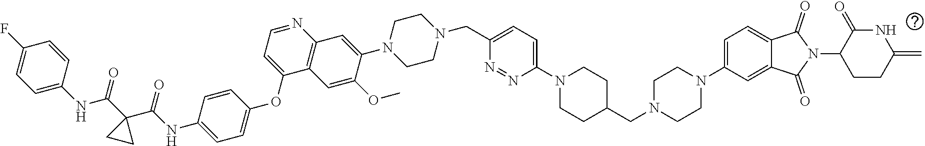 [SMILES: C=C1CCC(N2C(=O)c3ccc(N4CCN(CC5CCN(c6ccc(CN7CCN(c8cc9nccc(Oc%10ccc(NC(=O)C%11(C(=O)Nc%12ccc(F)cc%12)CC%11)cc%10)c9cc8OC)CC7)nn6)CC5)CC4)cc3C2=O)C(=O)N1]

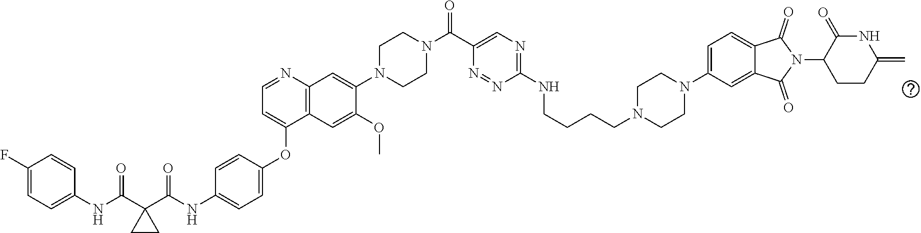 [SMILES: C=C1CCC(N2C(=O)c3ccc(N4CCN(CCCCNc5ncc(C(=O)N6CCN(c7cc8nccc(Oc9ccc(NC(=O)C%10(C(=O)Nc%11ccc(F)cc%11)CC%10)cc9)c8cc7OC)CC6)nn5)CC4)cc3C2=O)C(=O)N1]

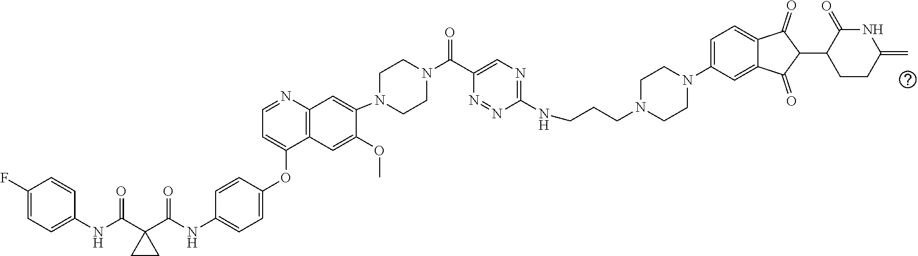 [SMILES: C=C1CCC(C2C(=O)c3ccc(N4CCN(CCCNc5ncc(C(=O)N6CCN(c7cc8nccc(Oc9ccc(NC(=O)C%10(C(=O)Nc%11ccc(F)cc%11)CC%10)cc9)c8cc7OC)CC6)nn5)CC4)cc3C2=O)C(=O)N1]

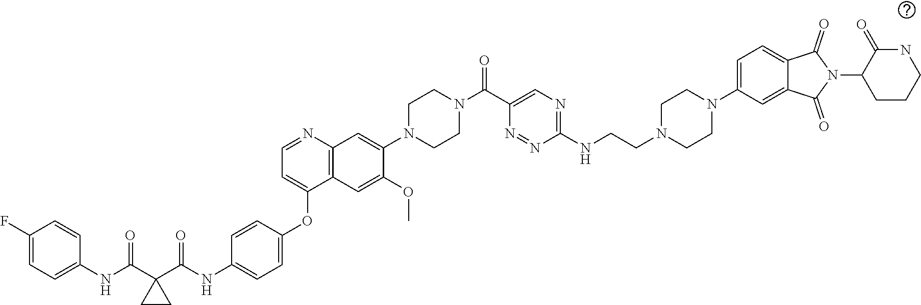 [SMILES: COc1cc2c(Oc3ccc(NC(=O)C4(C(=O)Nc5ccc(F)cc5)CC4)cc3)ccnc2cc1N1CCN(C(=O)c2cnc(NCCN3CCN(c4ccc5c(c4)C(=O)N(C4CCCNC4=O)C5=O)CC3)nn2)CC1]

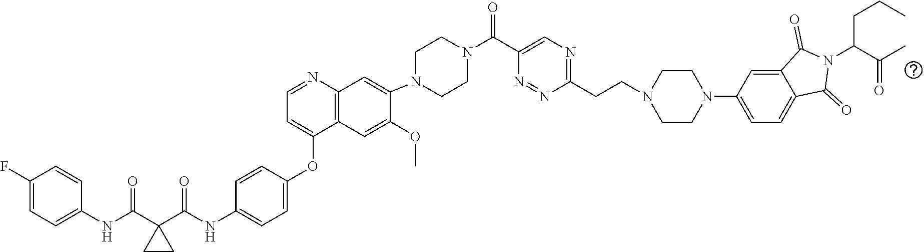 [SMILES: CCCC(C(C)=O)N1C(=O)c2ccc(N3CCN(CCc4ncc(C(=O)N5CCN(c6cc7nccc(Oc8ccc(NC(=O)C9(C(=O)Nc%10ccc(F)cc%10)CC9)cc8)c7cc6OC)CC5)nn4)CC3)cc2C1=O]

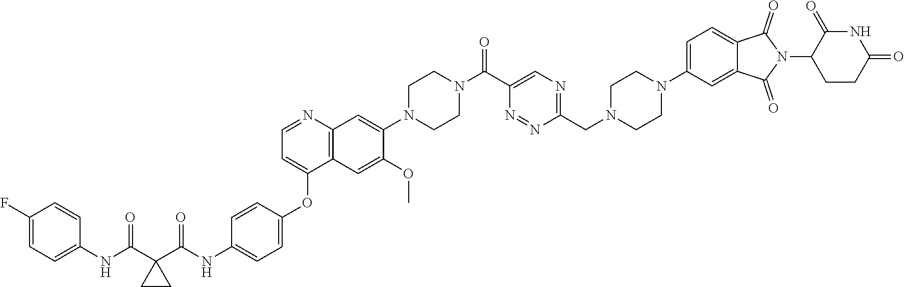 [SMILES: COc1cc2c(Oc3ccc(NC(=O)C4(C(=O)Nc5ccc(F)cc5)CC4)cc3)ccnc2cc1N1CCN(C(=O)c2cnc(CN3CCN(c4ccc5c(c4)C(=O)N(C4CCC(=O)NC4=O)C5=O)CC3)nn2)CC1]

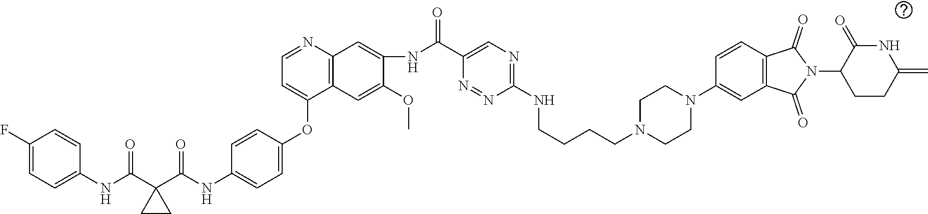 [SMILES: C=C1CCC(N2C(=O)c3ccc(N4CCN(CCCCNc5ncc(C(=O)Nc6cc7nccc(Oc8ccc(NC(=O)C9(C(=O)Nc%10ccc(F)cc%10)CC9)cc8)c7cc6OC)nn5)CC4)cc3C2=O)C(=O)N1]

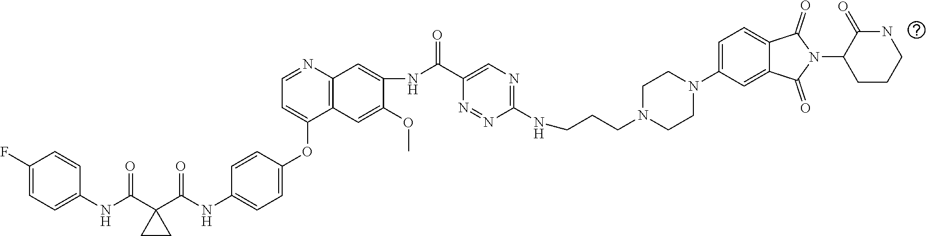 [SMILES: COc1cc2c(Oc3ccc(NC(=O)C4(C(=O)Nc5ccc(F)cc5)CC4)cc3)ccnc2cc1NC(=O)c1cnc(NCCCN2CCN(c3ccc4c(c3)C(=O)N(C3CCCNC3=O)C4=O)CC2)nn1]

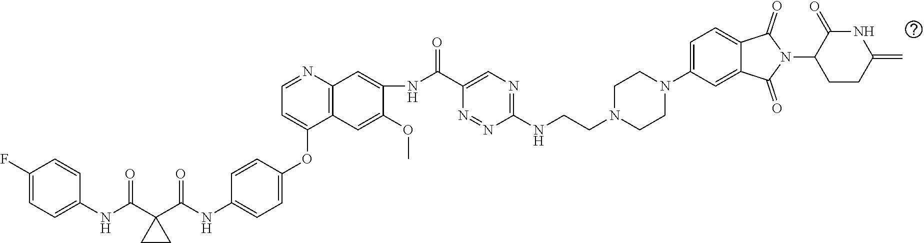 [SMILES: C=C1CCC(N2C(=O)c3ccc(N4CCN(CCNc5ncc(C(=O)Nc6cc7nccc(Oc8ccc(NC(=O)C9(C(=O)Nc%10ccc(F)cc%10)CC9)cc8)c7cc6OC)nn5)CC4)cc3C2=O)C(=O)N1]

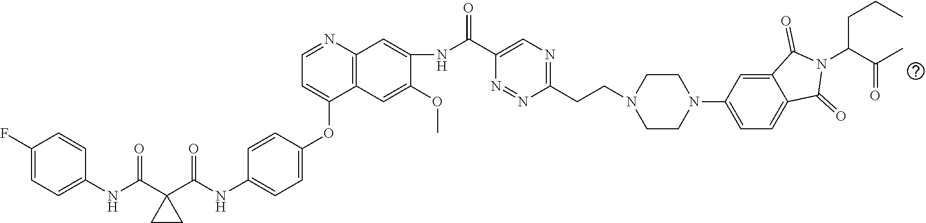 [SMILES: CCCC(C(C)=O)N1C(=O)c2ccc(N3CCN(CCc4ncc(C(=O)Nc5cc6nccc(Oc7ccc(NC(=O)C8(C(=O)Nc9ccc(F)cc9)CC8)cc7)c6cc5OC)nn4)CC3)cc2C1=O]

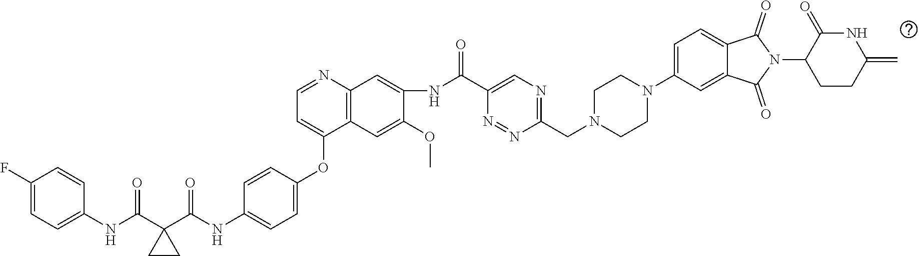 [SMILES: C=C1CCC(N2C(=O)c3ccc(N4CCN(Cc5ncc(C(=O)Nc6cc7nccc(Oc8ccc(NC(=O)C9(C(=O)Nc%10ccc(F)cc%10)CC9)cc8)c7cc6OC)nn5)CC4)cc3C2=O)C(=O)N1]

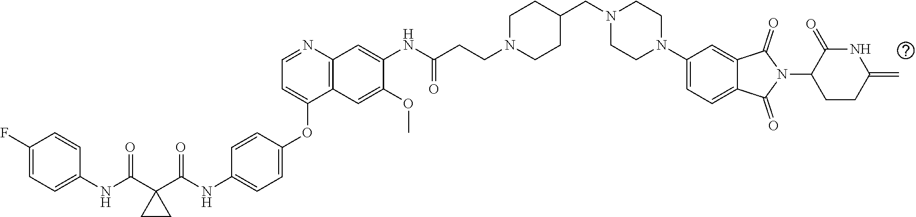 [SMILES: C=C1CCC(N2C(=O)c3ccc(N4CCN(CC5CCN(CCC(=O)Nc6cc7nccc(Oc8ccc(NC(=O)C9(C(=O)Nc%10ccc(F)cc%10)CC9)cc8)c7cc6OC)CC5)CC4)cc3C2=O)C(=O)N1]

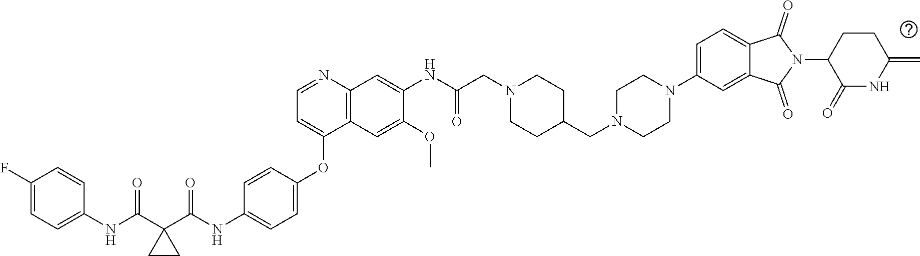 [SMILES: C=C1CCC(N2C(=O)c3ccc(N4CCN(CC5CCN(CC(=O)Nc6cc7nccc(Oc8ccc(NC(=O)C9(C(=O)Nc%10ccc(F)cc%10)CC9)cc8)c7cc6OC)CC5)CC4)cc3C2=O)C(=O)N1]

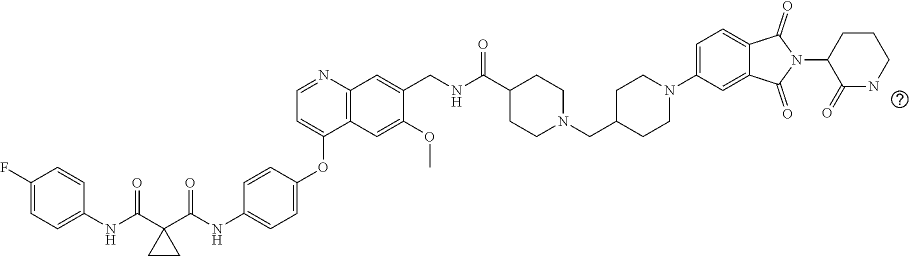 [SMILES: COc1cc2c(Oc3ccc(NC(=O)C4(C(=O)Nc5ccc(F)cc5)CC4)cc3)ccnc2cc1CNC(=O)C1CCN(CC2CCN(c3ccc4c(c3)C(=O)N(C3CCCNC3=O)C4=O)CC2)CC1]

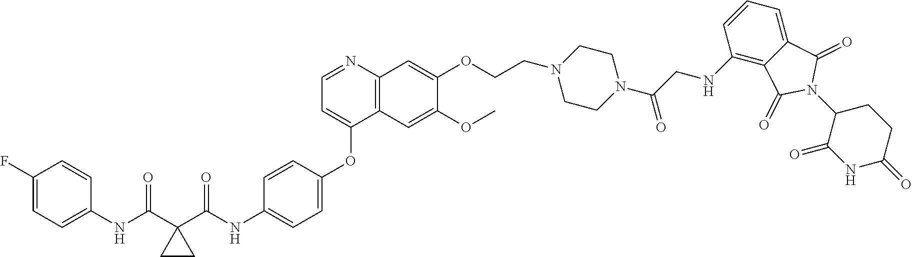 [SMILES: COc1cc2c(Oc3ccc(NC(=O)C4(C(=O)Nc5ccc(F)cc5)CC4)cc3)ccnc2cc1OCCN1CCN(C(=O)CNc2cccc3c2C(=O)N(C2CCC(=O)NC2=O)C3=O)CC1]

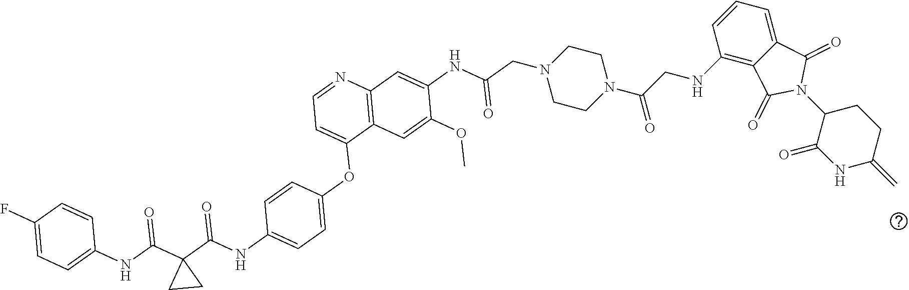 [SMILES: C=C1CCC(N2C(=O)c3cccc(NCC(=O)N4CCN(CC(=O)Nc5cc6nccc(Oc7ccc(NC(=O)C8(C(=O)Nc9ccc(F)cc9)CC8)cc7)c6cc5OC)CC4)c3C2=O)C(=O)N1]

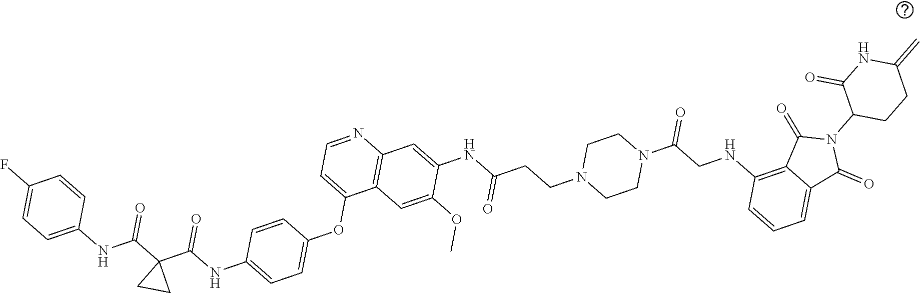 [SMILES: C=C1CCC(N2C(=O)c3cccc(NCC(=O)N4CCN(CCC(=O)Nc5cc6nccc(Oc7ccc(NC(=O)C8(C(=O)Nc9ccc(F)cc9)CC8)cc7)c6cc5OC)CC4)c3C2=O)C(=O)N1]

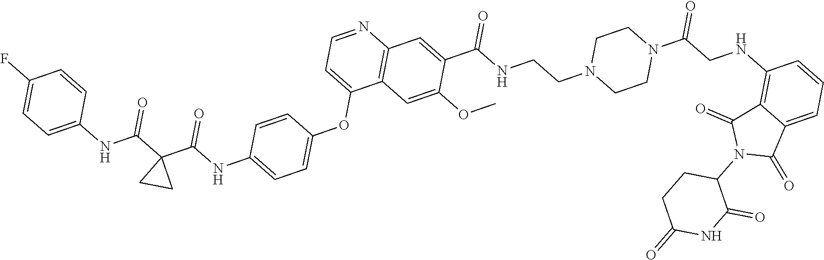 [SMILES: COc1cc2c(Oc3ccc(NC(=O)C4(C(=O)Nc5ccc(F)cc5)CC4)cc3)ccnc2cc1C(=O)NCCN1CCN(C(=O)CNc2cccc3c2C(=O)N(C2CCC(=O)NC2=O)C3=O)CC1]

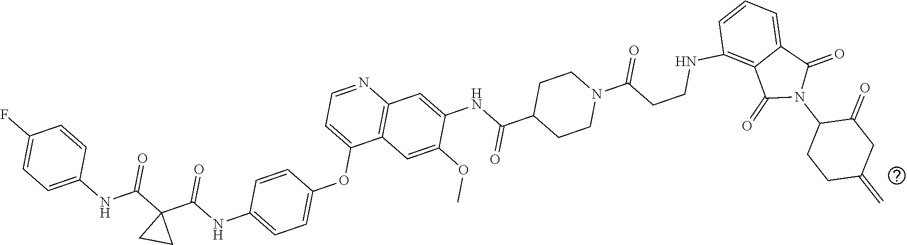 [SMILES: C=C1CCC(N2C(=O)c3cccc(NCCC(=O)N4CCC(C(=O)Nc5cc6nccc(Oc7ccc(NC(=O)C8(C(=O)Nc9ccc(F)cc9)CC8)cc7)c6cc5OC)CC4)c3C2=O)C(=O)C1]

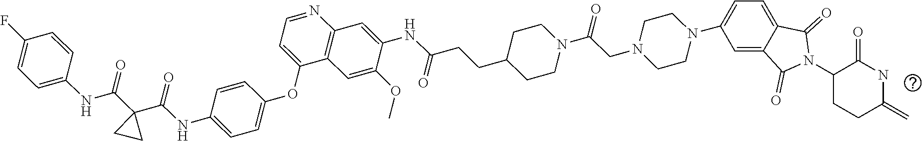 [SMILES: C=C1CCC(N2C(=O)c3ccc(N4CCN(CC(=O)N5CCC(CCC(=O)Nc6cc7nccc(Oc8ccc(NC(=O)C9(C(=O)Nc%10ccc(F)cc%10)CC9)cc8)c7cc6OC)CC5)CC4)cc3C2=O)C(=O)N1]

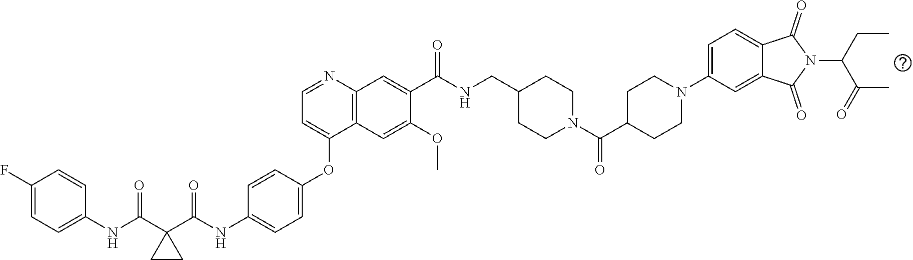 [SMILES: CCC(C(C)=O)N1C(=O)c2ccc(N3CCC(C(=O)N4CCC(CNC(=O)c5cc6nccc(Oc7ccc(NC(=O)C8(C(=O)Nc9ccc(F)cc9)CC8)cc7)c6cc5OC)CC4)CC3)cc2C1=O]

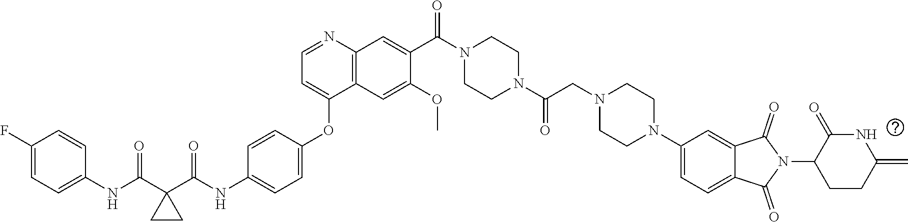 [SMILES: C=C1CCC(N2C(=O)c3ccc(N4CCN(CC(=O)N5CCN(C(=O)c6cc7nccc(Oc8ccc(NC(=O)C9(C(=O)Nc%10ccc(F)cc%10)CC9)cc8)c7cc6OC)CC5)CC4)cc3C2=O)C(=O)N1]

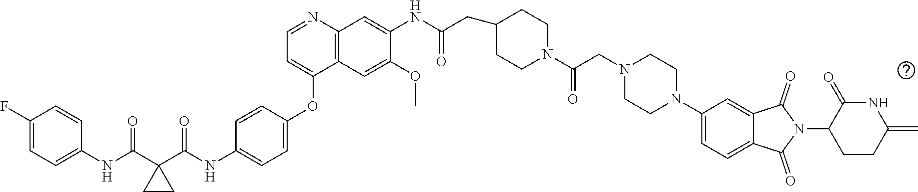 [SMILES: C=C1CCC(N2C(=O)c3ccc(N4CCN(CC(=O)N5CCC(CC(=O)Nc6cc7nccc(Oc8ccc(NC(=O)C9(C(=O)Nc%10ccc(F)cc%10)CC9)cc8)c7cc6OC)CC5)CC4)cc3C2=O)C(=O)N1]

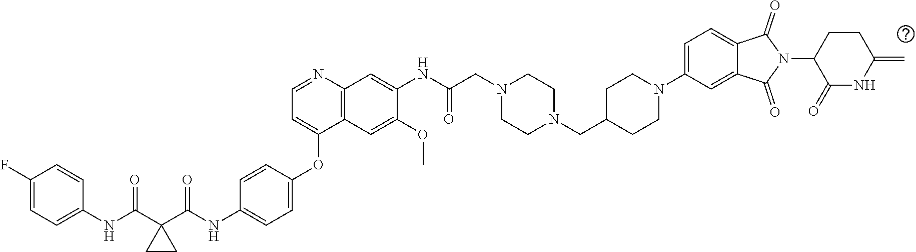 [SMILES: C=C1CCC(N2C(=O)c3ccc(N4CCC(CN5CCN(CC(=O)Nc6cc7nccc(Oc8ccc(NC(=O)C9(C(=O)Nc%10ccc(F)cc%10)CC9)cc8)c7cc6OC)CC5)CC4)cc3C2=O)C(=O)N1]

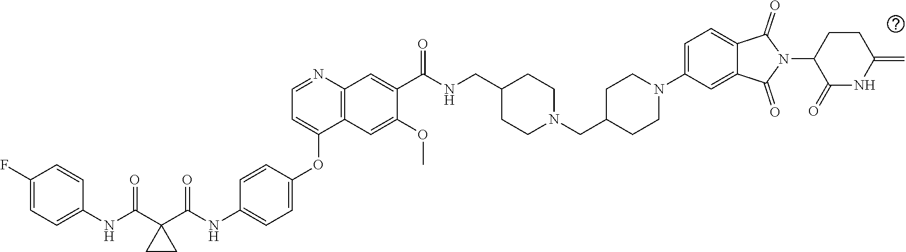 [SMILES: C=C1CCC(N2C(=O)c3ccc(N4CCC(CN5CCC(CNC(=O)c6cc7nccc(Oc8ccc(NC(=O)C9(C(=O)Nc%10ccc(F)cc%10)CC9)cc8)c7cc6OC)CC5)CC4)cc3C2=O)C(=O)N1]

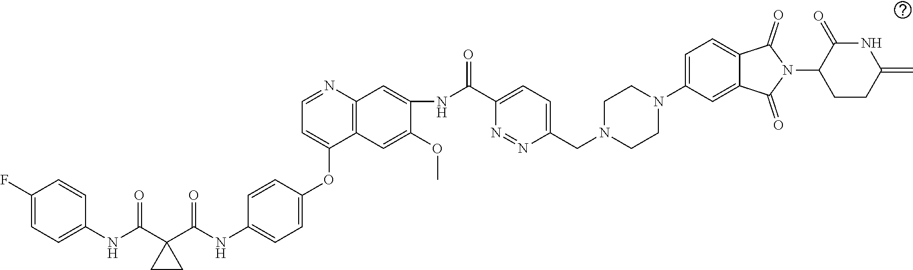 [SMILES: C=C1CCC(N2C(=O)c3ccc(N4CCN(Cc5ccc(C(=O)Nc6cc7nccc(Oc8ccc(NC(=O)C9(C(=O)Nc%10ccc(F)cc%10)CC9)cc8)c7cc6OC)nn5)CC4)cc3C2=O)C(=O)N1]

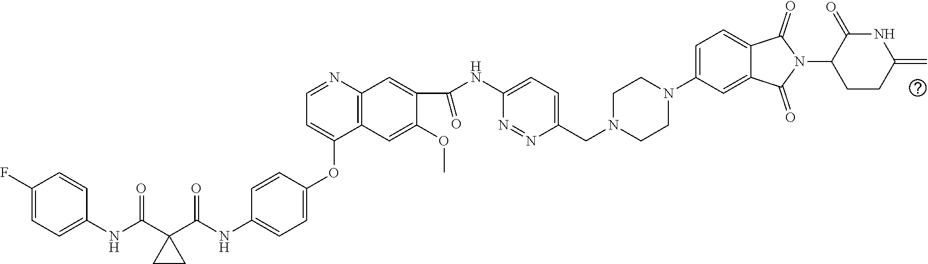 [SMILES: C=C1CCC(N2C(=O)c3ccc(N4CCN(Cc5ccc(NC(=O)c6cc7nccc(Oc8ccc(NC(=O)C9(C(=O)Nc%10ccc(F)cc%10)CC9)cc8)c7cc6OC)nn5)CC4)cc3C2=O)C(=O)N1]

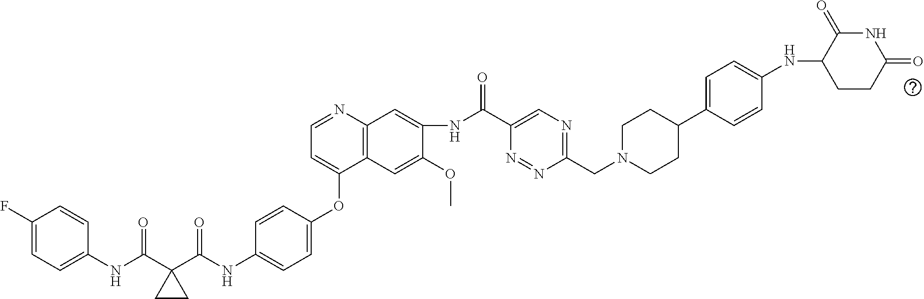 [SMILES: COc1cc2c(Oc3ccc(NC(=O)C4(C(=O)Nc5ccc(F)cc5)CC4)cc3)ccnc2cc1NC(=O)c1cnc(CN2CCC(c3ccc(NC4CCC(=O)NC4=O)cc3)CC2)nn1]

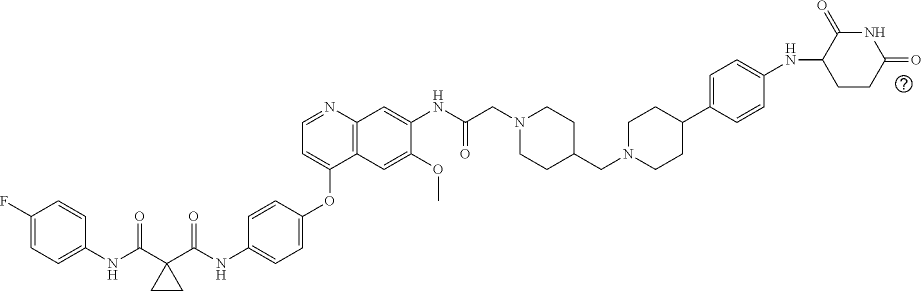 [SMILES: COc1cc2c(Oc3ccc(NC(=O)C4(C(=O)Nc5ccc(F)cc5)CC4)cc3)ccnc2cc1NC(=O)CN1CCC(CN2CCC(c3ccc(NC4CCC(=O)NC4=O)cc3)CC2)CC1]

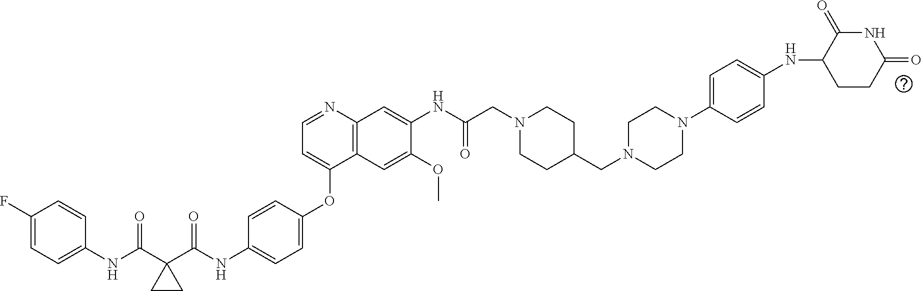 [SMILES: COc1cc2c(Oc3ccc(NC(=O)C4(C(=O)Nc5ccc(F)cc5)CC4)cc3)ccnc2cc1NC(=O)CN1CCC(CN2CCN(c3ccc(NC4CCC(=O)NC4=O)cc3)CC2)CC1]

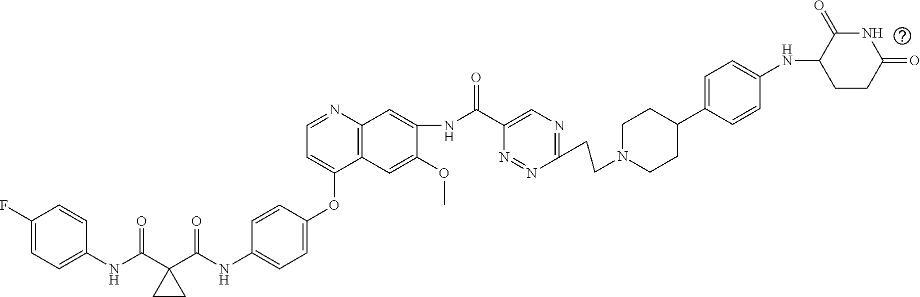 [SMILES: COc1cc2c(Oc3ccc(NC(=O)C4(C(=O)Nc5ccc(F)cc5)CC4)cc3)ccnc2cc1NC(=O)c1cnc(CCN2CCC(c3ccc(NC4CCC(=O)NC4=O)cc3)CC2)nn1]

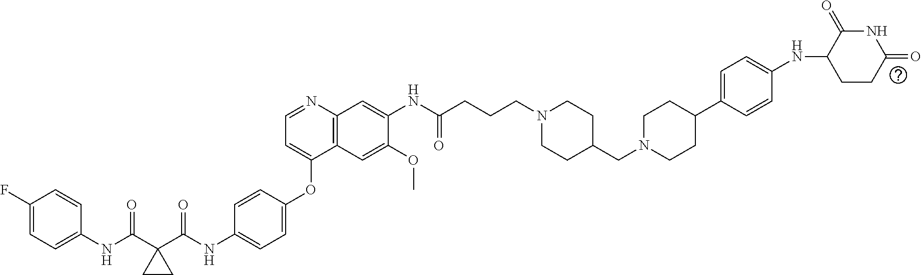 [SMILES: COc1cc2c(Oc3ccc(NC(=O)C4(C(=O)Nc5ccc(F)cc5)CC4)cc3)ccnc2cc1NC(=O)CCCN1CCC(CN2CCC(c3ccc(NC4CCC(=O)NC4=O)cc3)CC2)CC1]

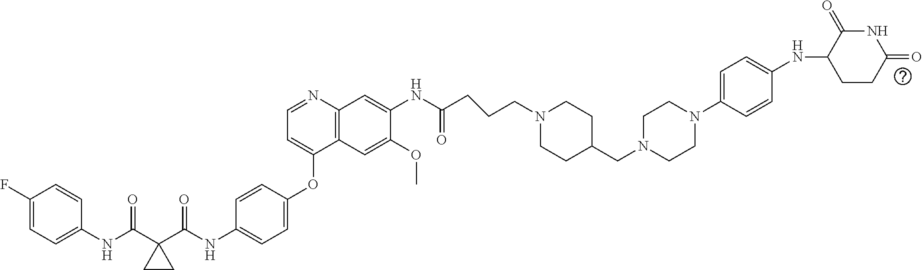 [SMILES: COc1cc2c(Oc3ccc(NC(=O)C4(C(=O)Nc5ccc(F)cc5)CC4)cc3)ccnc2cc1NC(=O)CCCN1CCC(CN2CCN(c3ccc(NC4CCC(=O)NC4=O)cc3)CC2)CC1]

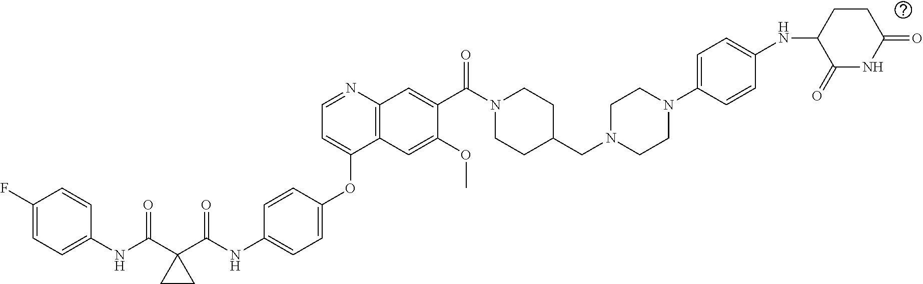 [SMILES: COc1cc2c(Oc3ccc(NC(=O)C4(C(=O)Nc5ccc(F)cc5)CC4)cc3)ccnc2cc1C(=O)N1CCC(CN2CCN(c3ccc(NC4CCC(=O)NC4=O)cc3)CC2)CC1]

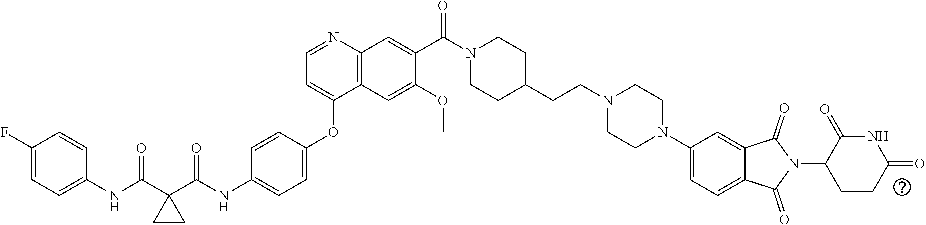 [SMILES: COc1cc2c(Oc3ccc(NC(=O)C4(C(=O)Nc5ccc(F)cc5)CC4)cc3)ccnc2cc1C(=O)N1CCC(CCN2CCN(c3ccc4c(c3)C(=O)N(C3CCC(=O)NC3=O)C4=O)CC2)CC1]

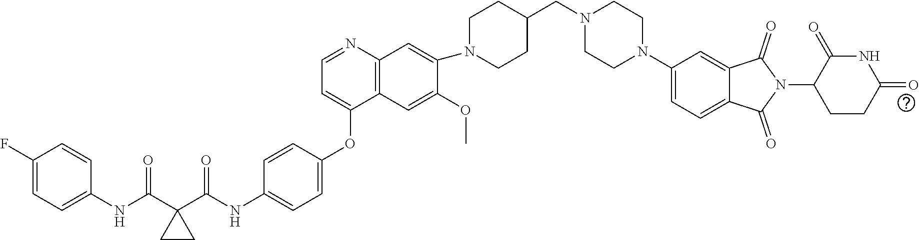 [SMILES: COc1cc2c(Oc3ccc(NC(=O)C4(C(=O)Nc5ccc(F)cc5)CC4)cc3)ccnc2cc1N1CCC(CN2CCN(c3ccc4c(c3)C(=O)N(C3CCC(=O)NC3=O)C4=O)CC2)CC1]

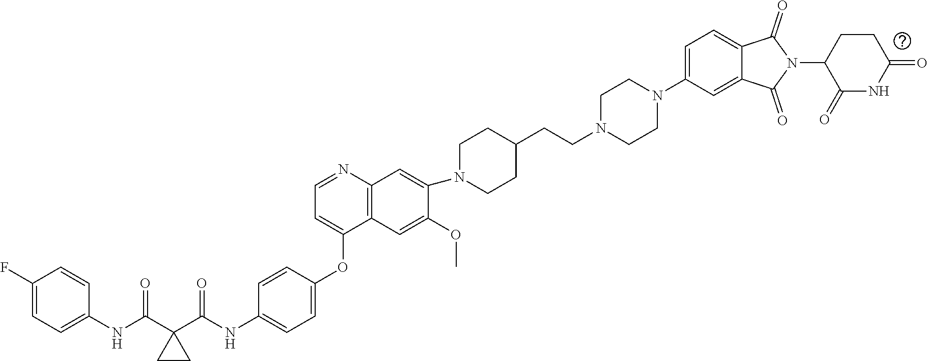 [SMILES: COc1cc2c(Oc3ccc(NC(=O)C4(C(=O)Nc5ccc(F)cc5)CC4)cc3)ccnc2cc1N1CCC(CCN2CCN(c3ccc4c(c3)C(=O)N(C3CCC(=O)NC3=O)C4=O)CC2)CC1]

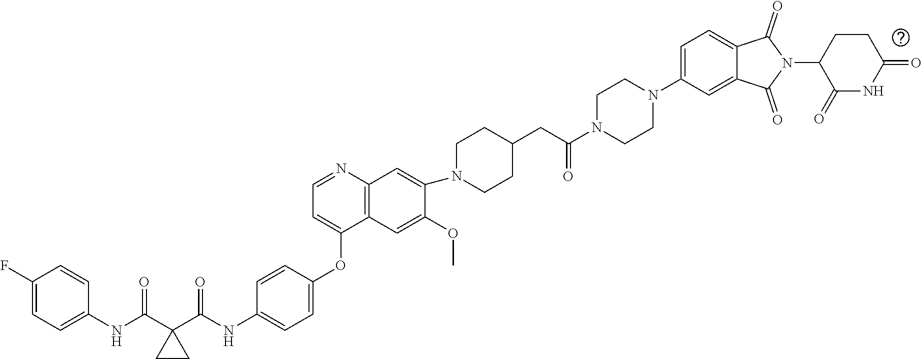 [SMILES: COc1cc2c(Oc3ccc(NC(=O)C4(C(=O)Nc5ccc(F)cc5)CC4)cc3)ccnc2cc1N1CCC(CC(=O)N2CCN(c3ccc4c(c3)C(=O)N(C3CCC(=O)NC3=O)C4=O)CC2)CC1]

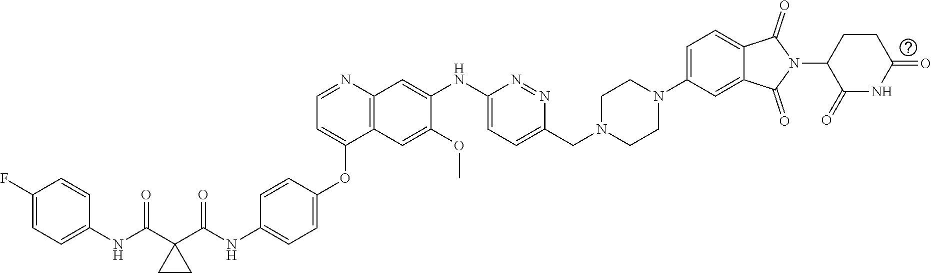 [SMILES: COc1cc2c(Oc3ccc(NC(=O)C4(C(=O)Nc5ccc(F)cc5)CC4)cc3)ccnc2cc1Nc1ccc(CN2CCN(c3ccc4c(c3)C(=O)N(C3CCC(=O)NC3=O)C4=O)CC2)nn1]

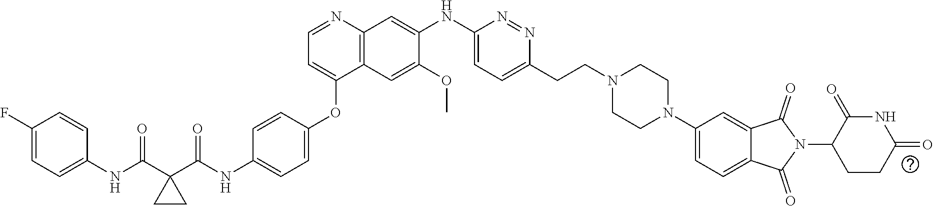 [SMILES: COc1cc2c(Oc3ccc(NC(=O)C4(C(=O)Nc5ccc(F)cc5)CC4)cc3)ccnc2cc1Nc1ccc(CCN2CCN(c3ccc4c(c3)C(=O)N(C3CCC(=O)NC3=O)C4=O)CC2)nn1]

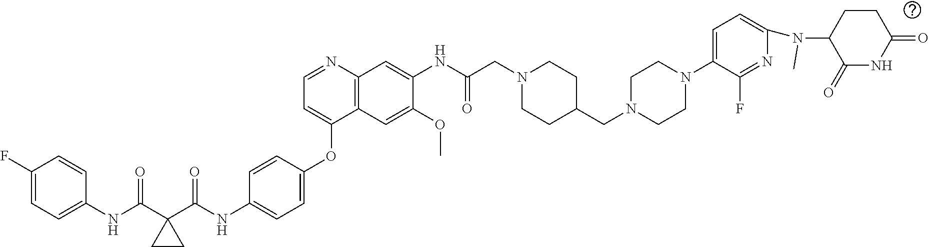 [SMILES: COc1cc2c(Oc3ccc(NC(=O)C4(C(=O)Nc5ccc(F)cc5)CC4)cc3)ccnc2cc1NC(=O)CN1CCC(CN2CCN(c3ccc(N(C)C4CCC(=O)NC4=O)nc3F)CC2)CC1]

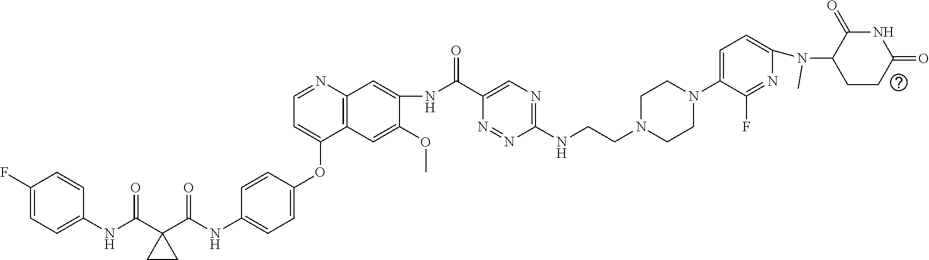 [SMILES: COc1cc2c(Oc3ccc(NC(=O)C4(C(=O)Nc5ccc(F)cc5)CC4)cc3)ccnc2cc1NC(=O)c1cnc(NCCN2CCN(c3ccc(N(C)C4CCC(=O)NC4=O)nc3F)CC2)nn1]

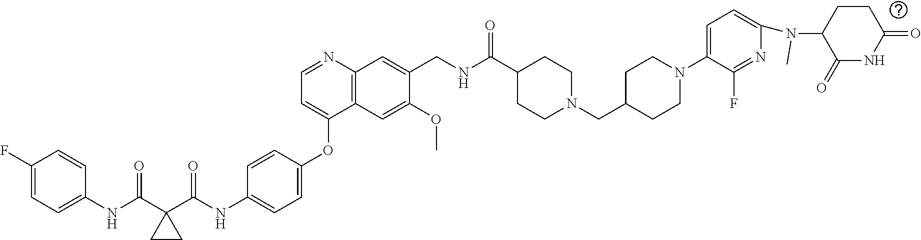 [SMILES: COc1cc2c(Oc3ccc(NC(=O)C4(C(=O)Nc5ccc(F)cc5)CC4)cc3)ccnc2cc1CNC(=O)C1CCN(CC2CCN(c3ccc(N(C)C4CCC(=O)NC4=O)nc3F)CC2)CC1]

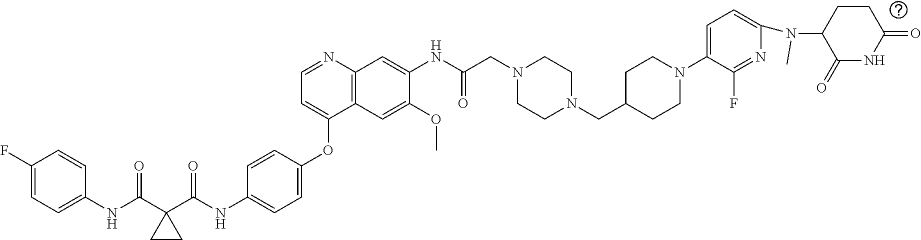 [SMILES: COc1cc2c(Oc3ccc(NC(=O)C4(C(=O)Nc5ccc(F)cc5)CC4)cc3)ccnc2cc1NC(=O)CN1CCN(CC2CCN(c3ccc(N(C)C4CCC(=O)NC4=O)nc3F)CC2)CC1]

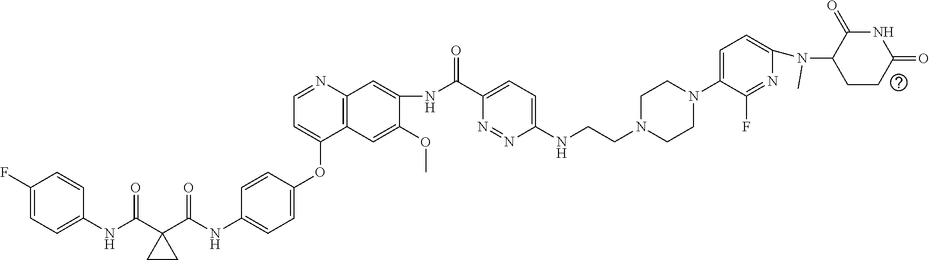 [SMILES: COc1cc2c(Oc3ccc(NC(=O)C4(C(=O)Nc5ccc(F)cc5)CC4)cc3)ccnc2cc1NC(=O)c1ccc(NCCN2CCN(c3ccc(N(C)C4CCC(=O)NC4=O)nc3F)CC2)nn1]

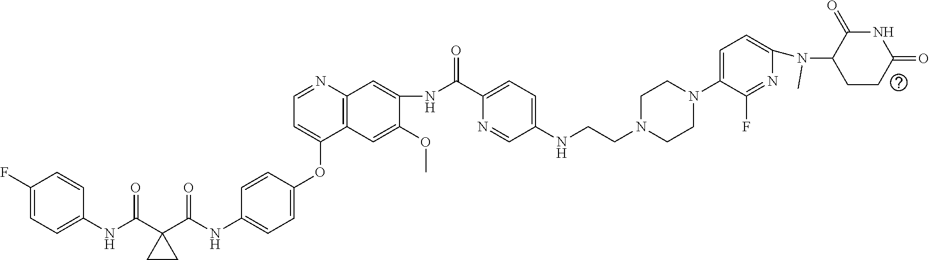 [SMILES: COc1cc2c(Oc3ccc(NC(=O)C4(C(=O)Nc5ccc(F)cc5)CC4)cc3)ccnc2cc1NC(=O)c1ccc(NCCN2CCN(c3ccc(N(C)C4CCC(=O)NC4=O)nc3F)CC2)cn1]

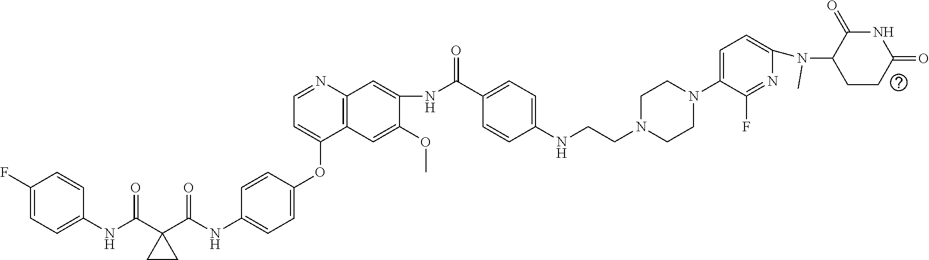 [SMILES: COc1cc2c(Oc3ccc(NC(=O)C4(C(=O)Nc5ccc(F)cc5)CC4)cc3)ccnc2cc1NC(=O)c1ccc(NCCN2CCN(c3ccc(N(C)C4CCC(=O)NC4=O)nc3F)CC2)cc1]

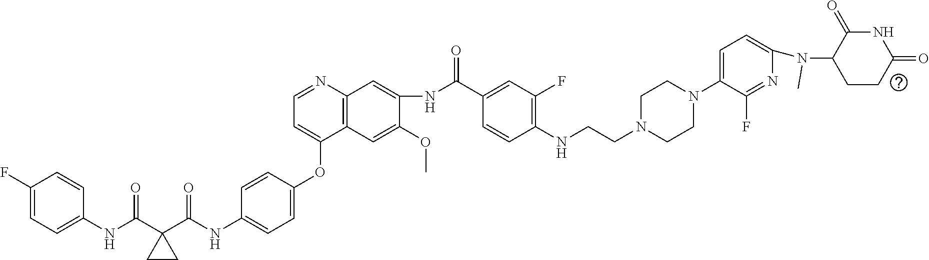 [SMILES: COc1cc2c(Oc3ccc(NC(=O)C4(C(=O)Nc5ccc(F)cc5)CC4)cc3)ccnc2cc1NC(=O)c1ccc(NCCN2CCN(c3ccc(N(C)C4CCC(=O)NC4=O)nc3F)CC2)c(F)c1]

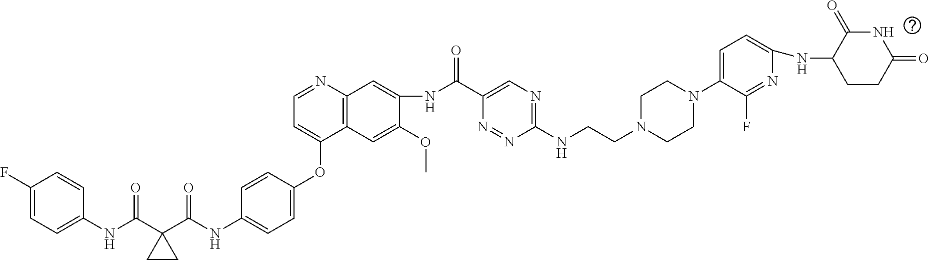 [SMILES: COc1cc2c(Oc3ccc(NC(=O)C4(C(=O)Nc5ccc(F)cc5)CC4)cc3)ccnc2cc1NC(=O)c1cnc(NCCN2CCN(c3ccc(NC4CCC(=O)NC4=O)nc3F)CC2)nn1]

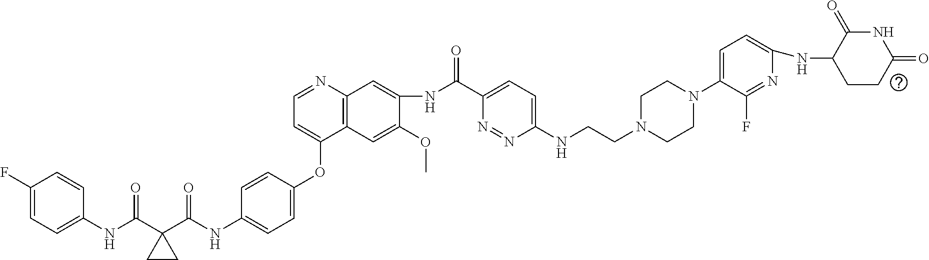 [SMILES: COc1cc2c(Oc3ccc(NC(=O)C4(C(=O)Nc5ccc(F)cc5)CC4)cc3)ccnc2cc1NC(=O)c1ccc(NCCN2CCN(c3ccc(NC4CCC(=O)NC4=O)nc3F)CC2)nn1]

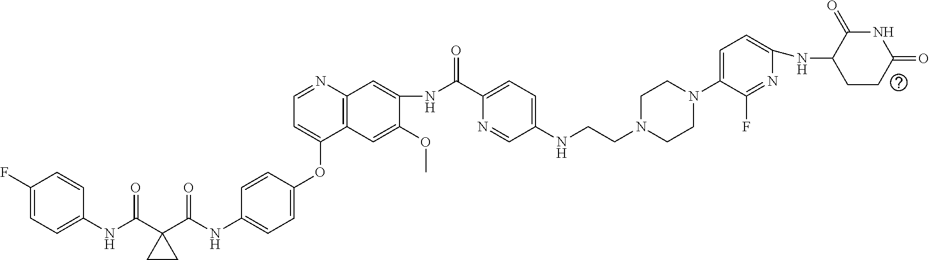 [SMILES: COc1cc2c(Oc3ccc(NC(=O)C4(C(=O)Nc5ccc(F)cc5)CC4)cc3)ccnc2cc1NC(=O)c1ccc(NCCN2CCN(c3ccc(NC4CCC(=O)NC4=O)nc3F)CC2)cn1]

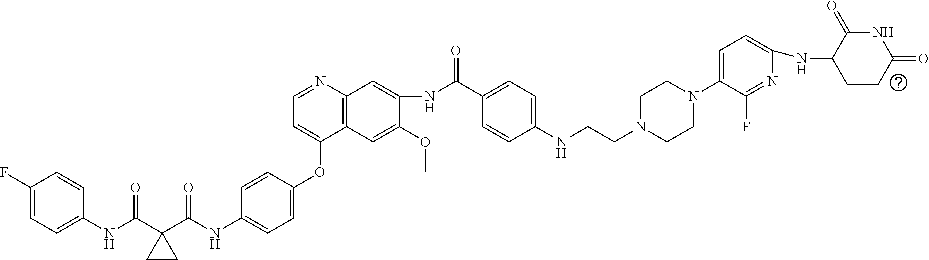 [SMILES: COc1cc2c(Oc3ccc(NC(=O)C4(C(=O)Nc5ccc(F)cc5)CC4)cc3)ccnc2cc1NC(=O)c1ccc(NCCN2CCN(c3ccc(NC4CCC(=O)NC4=O)nc3F)CC2)cc1]

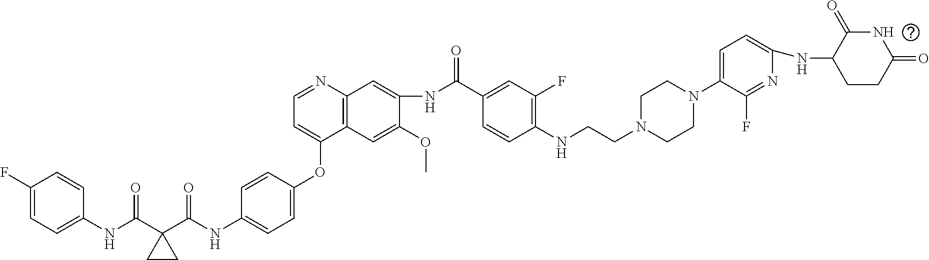 [SMILES: COc1cc2c(Oc3ccc(NC(=O)C4(C(=O)Nc5ccc(F)cc5)CC4)cc3)ccnc2cc1NC(=O)c1ccc(NCCN2CCN(c3ccc(NC4CCC(=O)NC4=O)nc3F)CC2)c(F)c1]

=0

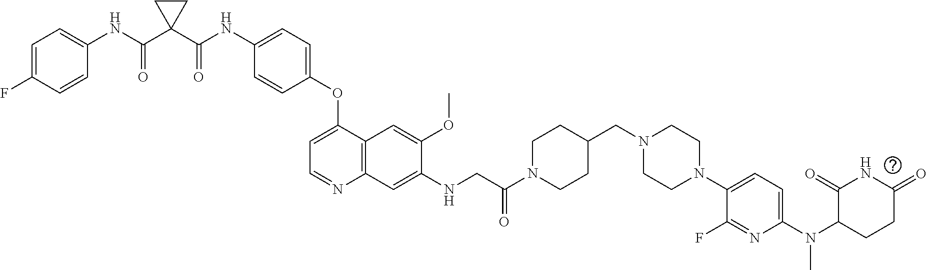 [SMILES: COc1cc2c(Oc3ccc(NC(=O)C4(C(=O)Nc5ccc(F)cc5)CC4)cc3)ccnc2cc1NCC(=O)N1CCC(CN2CCN(c3ccc(N(C)C4CCC(=O)NC4=O)nc3F)CC2)CC1]

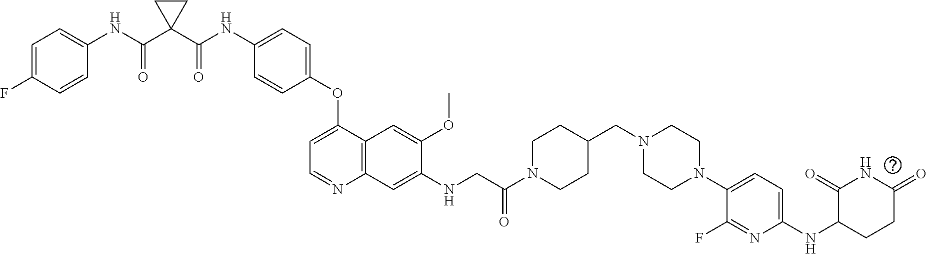 [SMILES: COc1cc2c(Oc3ccc(NC(=O)C4(C(=O)Nc5ccc(F)cc5)CC4)cc3)ccnc2cc1NCC(=O)N1CCC(CN2CCN(c3ccc(NC4CCC(=O)NC4=O)nc3F)CC2)CC1]

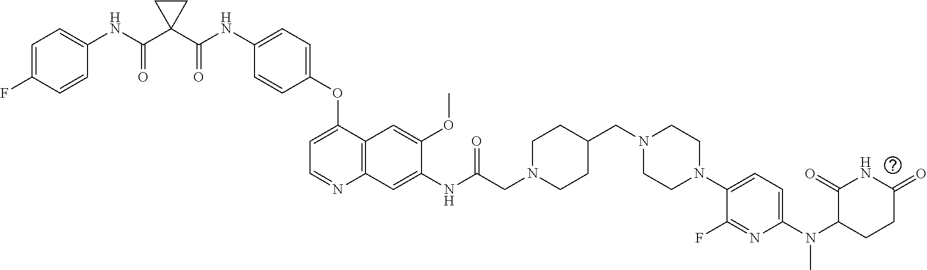 [SMILES: COc1cc2c(Oc3ccc(NC(=O)C4(C(=O)Nc5ccc(F)cc5)CC4)cc3)ccnc2cc1NC(=O)CN1CCC(CN2CCN(c3ccc(N(C)C4CCC(=O)NC4=O)nc3F)CC2)CC1]

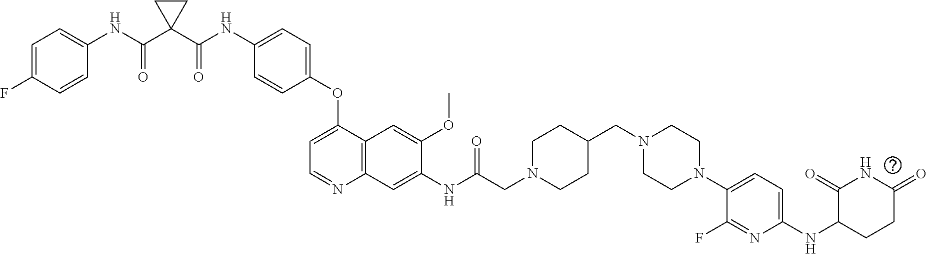 [SMILES: COc1cc2c(Oc3ccc(NC(=O)C4(C(=O)Nc5ccc(F)cc5)CC4)cc3)ccnc2cc1NC(=O)CN1CCC(CN2CCN(c3ccc(NC4CCC(=O)NC4=O)nc3F)CC2)CC1]

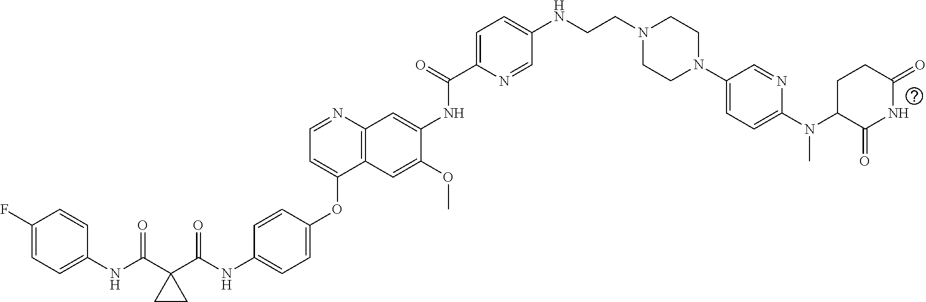 [SMILES: COc1cc2c(Oc3ccc(NC(=O)C4(C(=O)Nc5ccc(F)cc5)CC4)cc3)ccnc2cc1NC(=O)c1ccc(NCCN2CCN(c3ccc(N(C)C4CCC(=O)NC4=O)nc3)CC2)cn1]

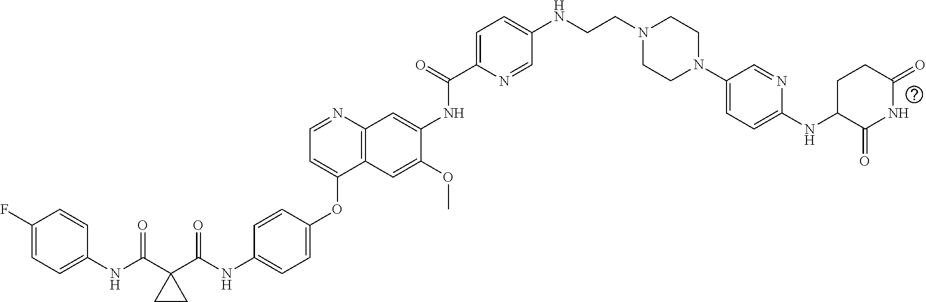 [SMILES: COc1cc2c(Oc3ccc(NC(=O)C4(C(=O)Nc5ccc(F)cc5)CC4)cc3)ccnc2cc1NC(=O)c1ccc(NCCN2CCN(c3ccc(NC4CCC(=O)NC4=O)nc3)CC2)cn1]

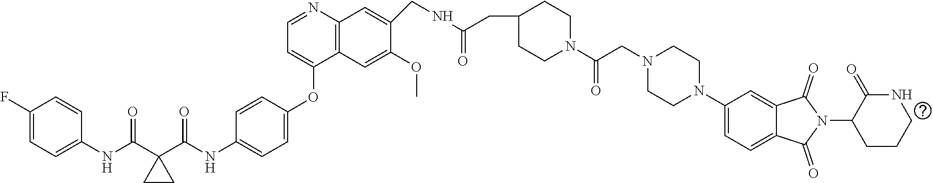 [SMILES: COc1cc2c(Oc3ccc(NC(=O)C4(C(=O)Nc5ccc(F)cc5)CC4)cc3)ccnc2cc1CNC(=O)CC1CCN(C(=O)CN2CCN(c3ccc4c(c3)C(=O)N(C3CCCNC3=O)C4=O)CC2)CC1]

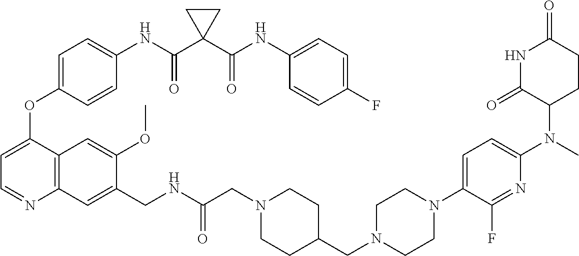 [SMILES: COc1cc2c(Oc3ccc(NC(=O)C4(C(=O)Nc5ccc(F)cc5)CC4)cc3)ccnc2cc1CNC(=O)CN1CCC(CN2CCN(c3ccc(N(C)C4CCC(=O)NC4=O)nc3F)CC2)CC1]

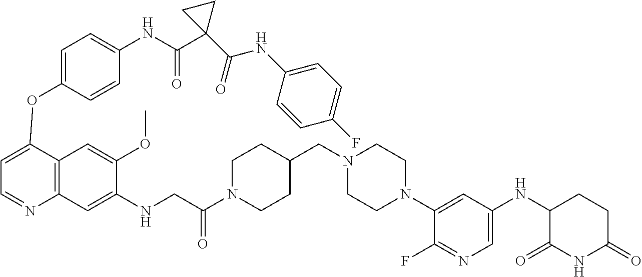 [SMILES: COc1cc2c(Oc3ccc(NC(=O)C4(C(=O)Nc5ccc(F)cc5)CC4)cc3)ccnc2cc1NCC(=O)N1CCC(CN2CCN(c3cc(NC4CCC(=O)NC4=O)cnc3F)CC2)CC1]

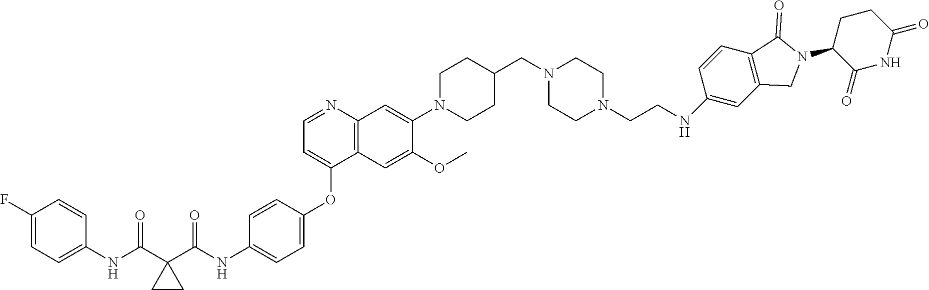 [SMILES: COc1cc2c(Oc3ccc(NC(=O)C4(C(=O)Nc5ccc(F)cc5)CC4)cc3)ccnc2cc1N1CCC(CN2CCN(CCNc3ccc4c(c3)CN([C@H]3CCC(=O)NC3=O)C4=O)CC2)CC1]

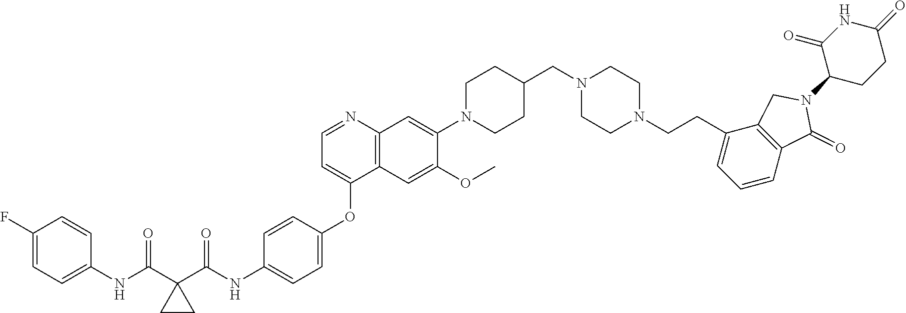 [SMILES: COc1cc2c(Oc3ccc(NC(=O)C4(C(=O)Nc5ccc(F)cc5)CC4)cc3)ccnc2cc1N1CCC(CN2CCN(CCc3cccc4c3CN([C@@H]3CCC(=O)NC3=O)C4=O)CC2)CC1]

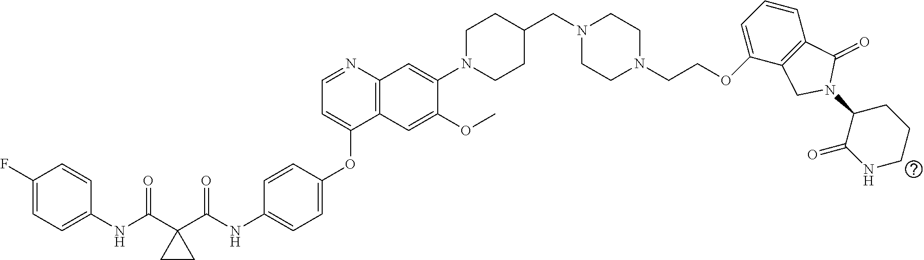 [SMILES: COc1cc2c(Oc3ccc(NC(=O)C4(C(=O)Nc5ccc(F)cc5)CC4)cc3)ccnc2cc1N1CCC(CN2CCN(CCOc3cccc4c3CN([C@H]3CCCNC3=O)C4=O)CC2)CC1]

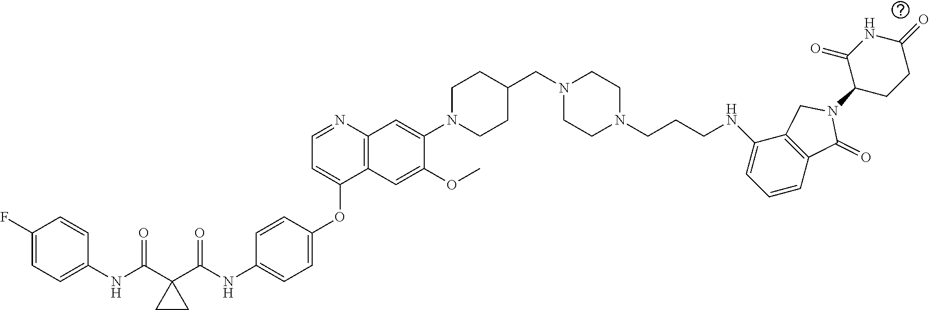 [SMILES: COc1cc2c(Oc3ccc(NC(=O)C4(C(=O)Nc5ccc(F)cc5)CC4)cc3)ccnc2cc1N1CCC(CN2CCN(CCCNc3cccc4c3CN([C@@H]3CCC(=O)NC3=O)C4=O)CC2)CC1]

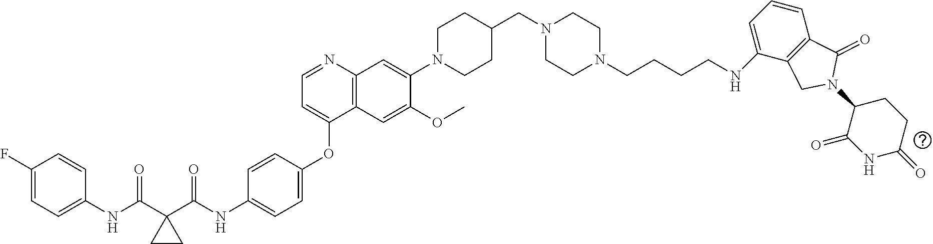 [SMILES: COc1cc2c(Oc3ccc(NC(=O)C4(C(=O)Nc5ccc(F)cc5)CC4)cc3)ccnc2cc1N1CCC(CN2CCN(CCCCNc3cccc4c3CN([C@H]3CCC(=O)NC3=O)C4=O)CC2)CC1]

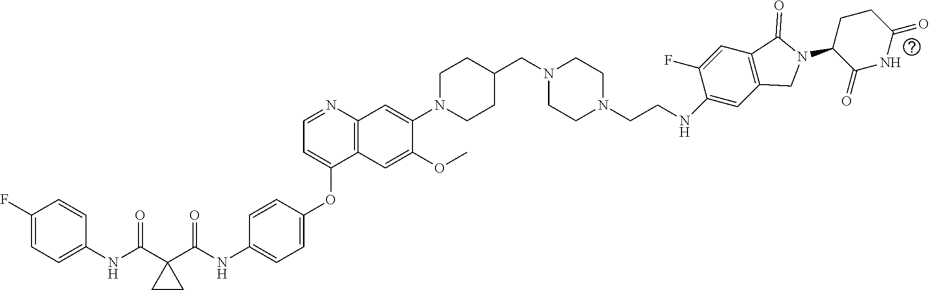 [SMILES: COc1cc2c(Oc3ccc(NC(=O)C4(C(=O)Nc5ccc(F)cc5)CC4)cc3)ccnc2cc1N1CCC(CN2CCN(CCNc3cc4c(cc3F)C(=O)N([C@H]3CCC(=O)NC3=O)C4)CC2)CC1]

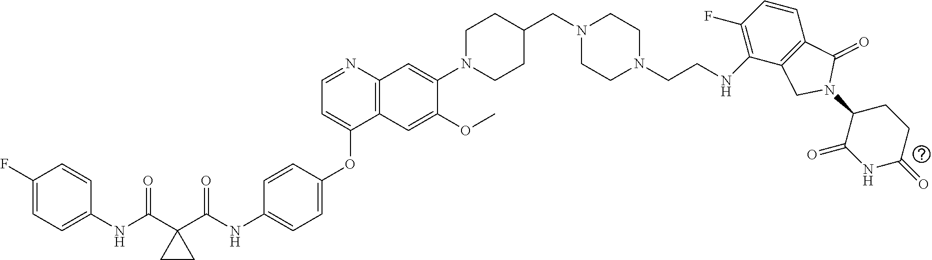 [SMILES: COc1cc2c(Oc3ccc(NC(=O)C4(C(=O)Nc5ccc(F)cc5)CC4)cc3)ccnc2cc1N1CCC(CN2CCN(CCNc3c(F)ccc4c3CN([C@H]3CCC(=O)NC3=O)C4=O)CC2)CC1]

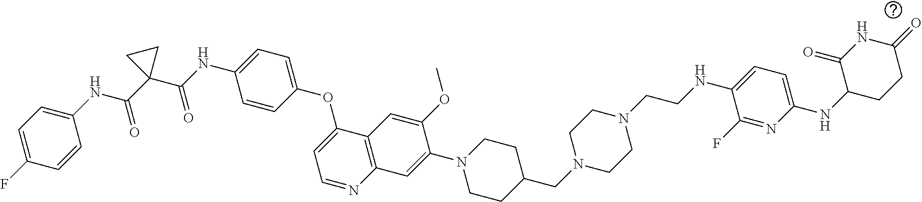 [SMILES: COc1cc2c(Oc3ccc(NC(=O)C4(C(=O)Nc5ccc(F)cc5)CC4)cc3)ccnc2cc1N1CCC(CN2CCN(CCNc3ccc(NC4CCC(=O)NC4=O)nc3F)CC2)CC1]

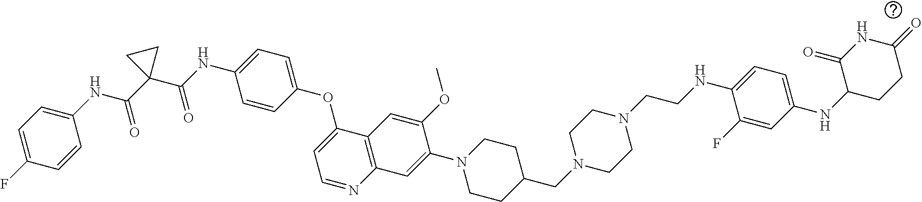 [SMILES: COc1cc2c(Oc3ccc(NC(=O)C4(C(=O)Nc5ccc(F)cc5)CC4)cc3)ccnc2cc1N1CCC(CN2CCN(CCNc3ccc(NC4CCC(=O)NC4=O)cc3F)CC2)CC1]

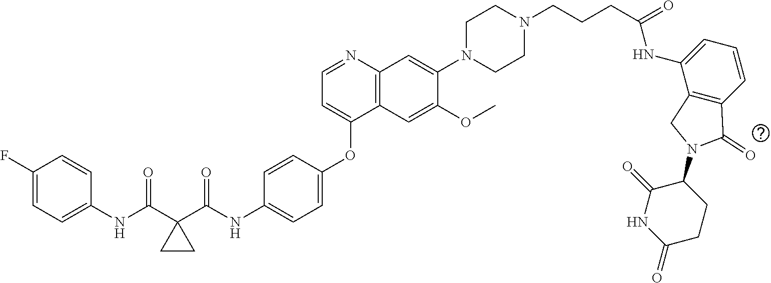 [SMILES: COc1cc2c(Oc3ccc(NC(=O)C4(C(=O)Nc5ccc(F)cc5)CC4)cc3)ccnc2cc1N1CCN(CCCC(=O)Nc2cccc3c2CN([C@H]2CCC(=O)NC2=O)C3=O)CC1]

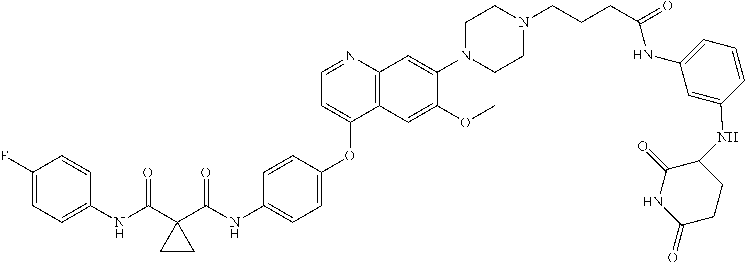 [SMILES: COc1cc2c(Oc3ccc(NC(=O)C4(C(=O)Nc5ccc(F)cc5)CC4)cc3)ccnc2cc1N1CCN(CCCC(=O)Nc2cccc(NC3CCC(=O)NC3=O)c2)CC1]

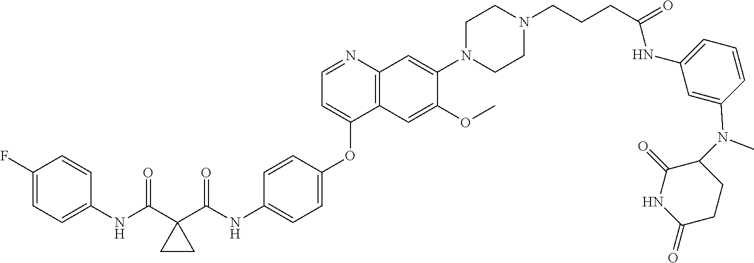 [SMILES: COc1cc2c(Oc3ccc(NC(=O)C4(C(=O)Nc5ccc(F)cc5)CC4)cc3)ccnc2cc1N1CCN(CCCC(=O)Nc2cccc(N(C)C3CCC(=O)NC3=O)c2)CC1]

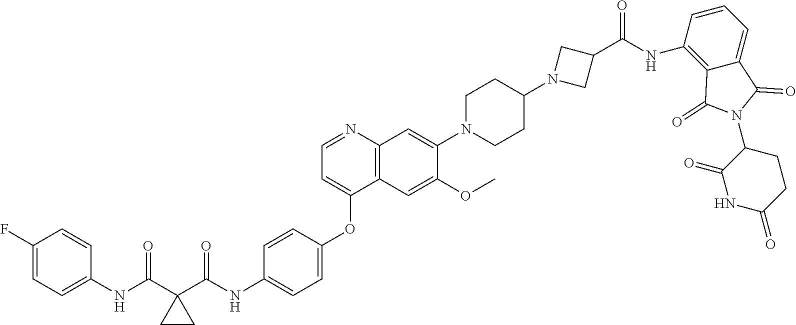 [SMILES: COc1cc2c(Oc3ccc(NC(=O)C4(C(=O)Nc5ccc(F)cc5)CC4)cc3)ccnc2cc1N1CCC(N2CC(C(=O)Nc3cccc4c3C(=O)N(C3CCC(=O)NC3=O)C4=O)C2)CC1]

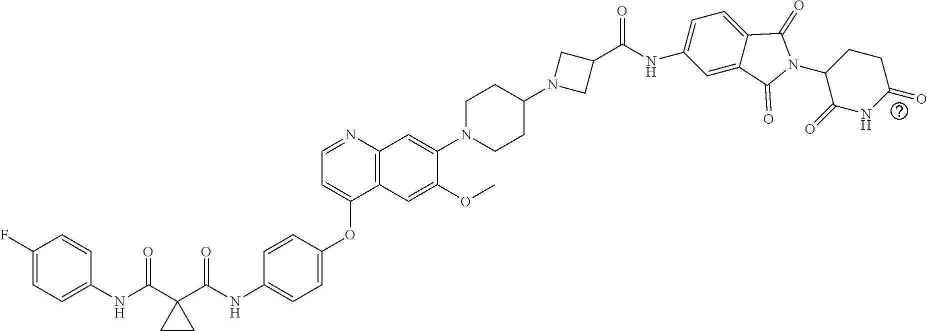 [SMILES: COc1cc2c(Oc3ccc(NC(=O)C4(C(=O)Nc5ccc(F)cc5)CC4)cc3)ccnc2cc1N1CCC(N2CC(C(=O)Nc3ccc4c(c3)C(=O)N(C3CCC(=O)NC3=O)C4=O)C2)CC1]

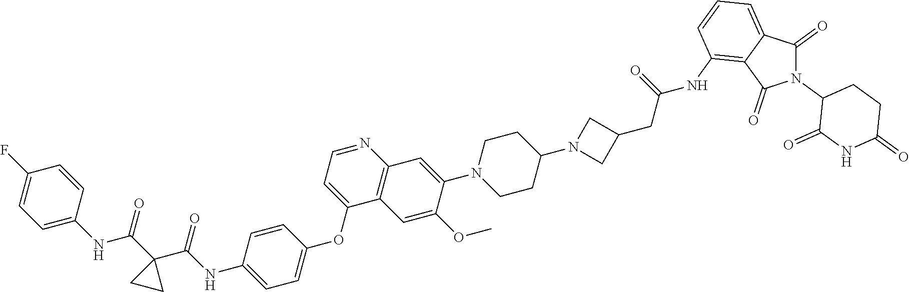 [SMILES: COc1cc2c(Oc3ccc(NC(=O)C4(C(=O)Nc5ccc(F)cc5)CC4)cc3)ccnc2cc1N1CCC(N2CC(CC(=O)Nc3cccc4c3C(=O)N(C3CCC(=O)NC3=O)C4=O)C2)CC1]

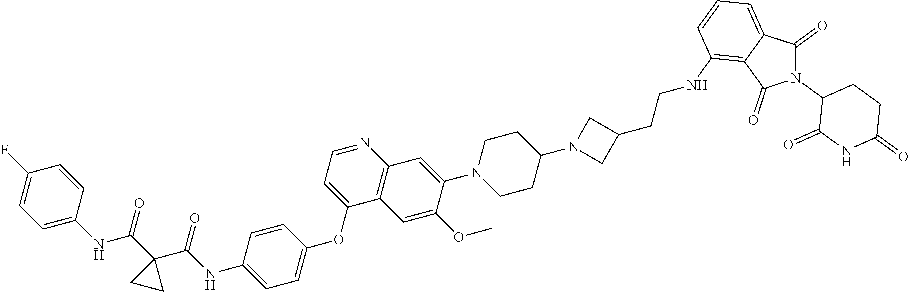 [SMILES: COc1cc2c(Oc3ccc(NC(=O)C4(C(=O)Nc5ccc(F)cc5)CC4)cc3)ccnc2cc1N1CCC(N2CC(CCNc3cccc4c3C(=O)N(C3CCC(=O)NC3=O)C4=O)C2)CC1]

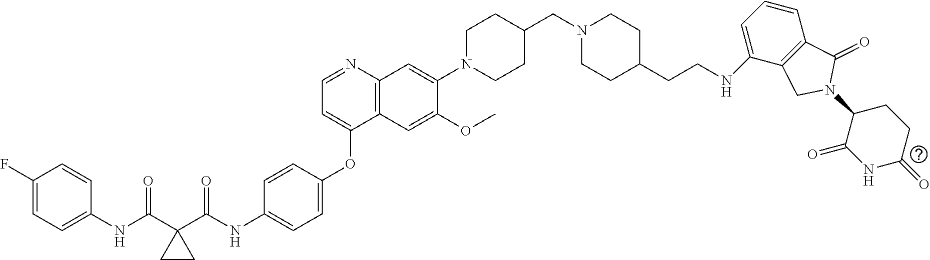 [SMILES: COc1cc2c(Oc3ccc(NC(=O)C4(C(=O)Nc5ccc(F)cc5)CC4)cc3)ccnc2cc1N1CCC(CN2CCC(CCNc3cccc4c3CN([C@H]3CCC(=O)NC3=O)C4=O)CC2)CC1]

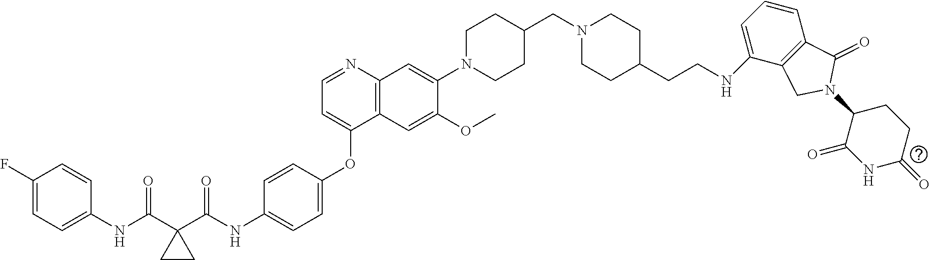 [SMILES: COc1cc2c(Oc3ccc(NC(=O)C4(C(=O)Nc5ccc(F)cc5)CC4)cc3)ccnc2cc1N1CCC(CN2CCC(CCNc3cccc4c3CN([C@H]3CCC(=O)NC3=O)C4=O)CC2)CC1]

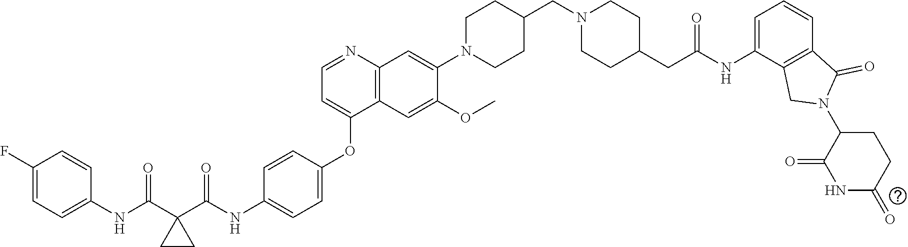 [SMILES: COc1cc2c(Oc3ccc(NC(=O)C4(C(=O)Nc5ccc(F)cc5)CC4)cc3)ccnc2cc1N1CCC(CN2CCC(CC(=O)Nc3cccc4c3CN(C3CCC(=O)NC3=O)C4=O)CC2)CC1]

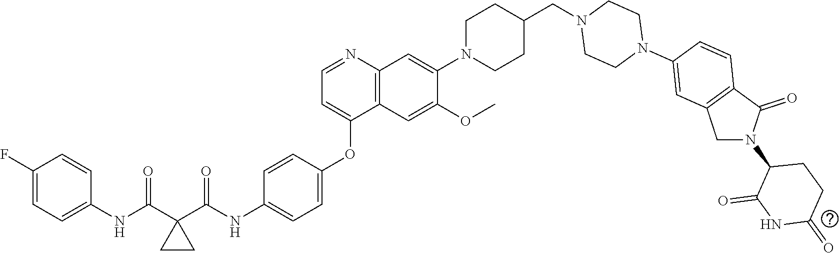 [SMILES: COc1cc2c(Oc3ccc(NC(=O)C4(C(=O)Nc5ccc(F)cc5)CC4)cc3)ccnc2cc1N1CCC(CN2CCN(c3ccc4c(c3)CN([C@H]3CCC(=O)NC3=O)C4=O)CC2)CC1]

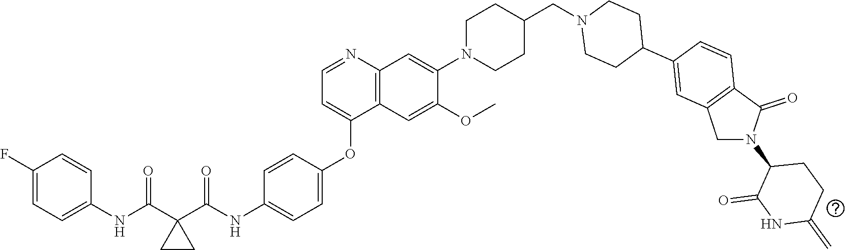 [SMILES: C=C1CC[C@H](N2Cc3cc(C4CCN(CC5CCN(c6cc7nccc(Oc8ccc(NC(=O)C9(C(=O)Nc%10ccc(F)cc%10)CC9)cc8)c7cc6OC)CC5)CC4)ccc3C2=O)C(=O)N1]

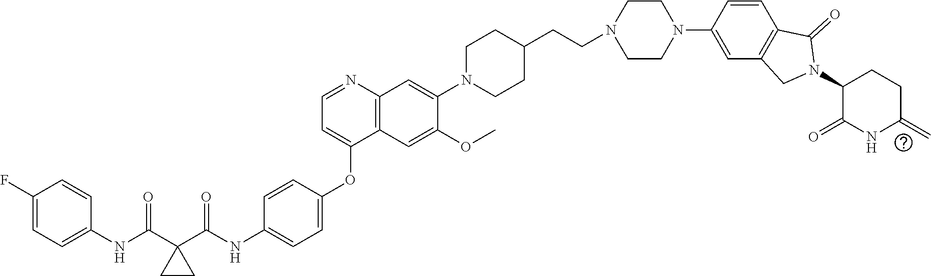 [SMILES: C=C1CC[C@H](N2Cc3cc(N4CCN(CCC5CCN(c6cc7nccc(Oc8ccc(NC(=O)C9(C(=O)Nc%10ccc(F)cc%10)CC9)cc8)c7cc6OC)CC5)CC4)ccc3C2=O)C(=O)N1]

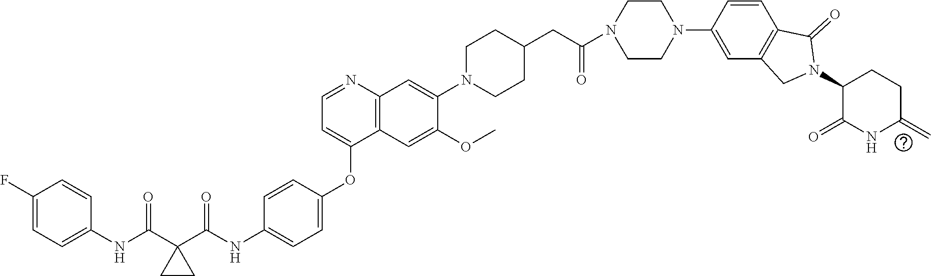 [SMILES: C=C1CC[C@H](N2Cc3cc(N4CCN(C(=O)CC5CCN(c6cc7nccc(Oc8ccc(NC(=O)C9(C(=O)Nc%10ccc(F)cc%10)CC9)cc8)c7cc6OC)CC5)CC4)ccc3C2=O)C(=O)N1]

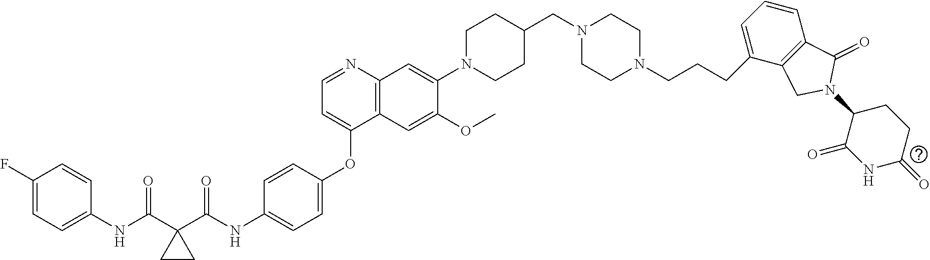 [SMILES: COc1cc2c(Oc3ccc(NC(=O)C4(C(=O)Nc5ccc(F)cc5)CC4)cc3)ccnc2cc1N1CCC(CN2CCN(CCCc3cccc4c3CN([C@H]3CCC(=O)NC3=O)C4=O)CC2)CC1]

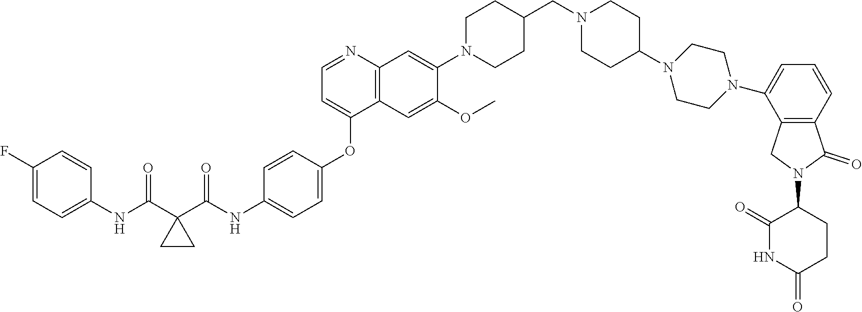 [SMILES: COc1cc2c(Oc3ccc(NC(=O)C4(C(=O)Nc5ccc(F)cc5)CC4)cc3)ccnc2cc1N1CCC(CN2CCC(N3CCN(c4cccc5c4CN([C@H]4CCC(=O)NC4=O)C5=O)CC3)CC2)CC1]

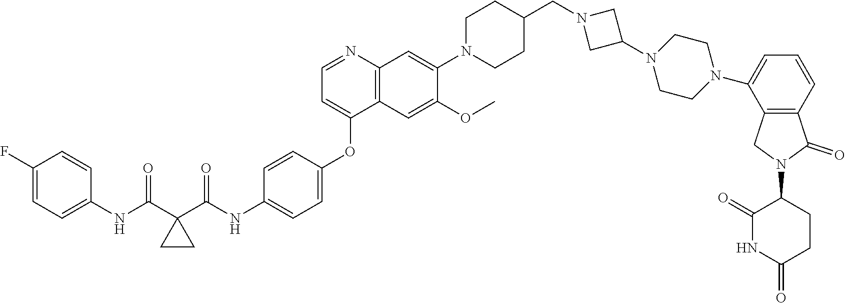 [SMILES: COc1cc2c(Oc3ccc(NC(=O)C4(C(=O)Nc5ccc(F)cc5)CC4)cc3)ccnc2cc1N1CCC(CN2CC(N3CCN(c4cccc5c4CN([C@H]4CCC(=O)NC4=O)C5=O)CC3)C2)CC1]

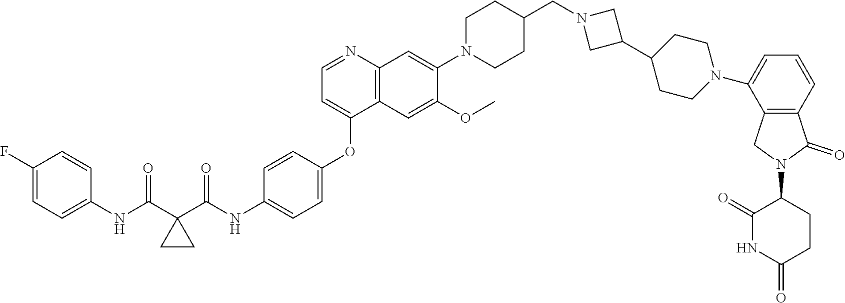 [SMILES: COc1cc2c(Oc3ccc(NC(=O)C4(C(=O)Nc5ccc(F)cc5)CC4)cc3)ccnc2cc1N1CCC(CN2CC(C3CCN(c4cccc5c4CN([C@H]4CCC(=O)NC4=O)C5=O)CC3)C2)CC1]

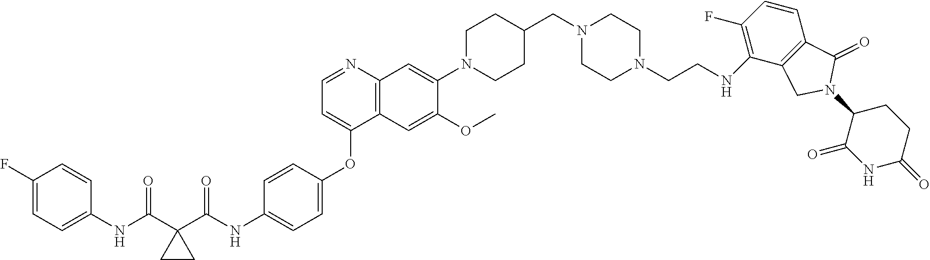 [SMILES: COc1cc2c(Oc3ccc(NC(=O)C4(C(=O)Nc5ccc(F)cc5)CC4)cc3)ccnc2cc1N1CCC(CN2CCN(CCNc3c(F)ccc4c3CN([C@H]3CCC(=O)NC3=O)C4=O)CC2)CC1]

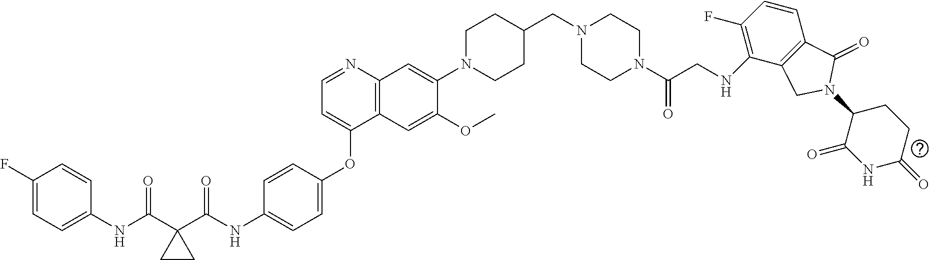 [SMILES: COc1cc2c(Oc3ccc(NC(=O)C4(C(=O)Nc5ccc(F)cc5)CC4)cc3)ccnc2cc1N1CCC(CN2CCN(C(=O)CNc3c(F)ccc4c3CN([C@H]3CCC(=O)NC3=O)C4=O)CC2)CC1]

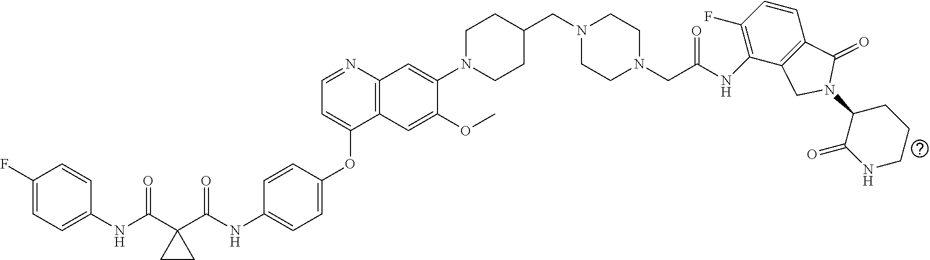 [SMILES: COc1cc2c(Oc3ccc(NC(=O)C4(C(=O)Nc5ccc(F)cc5)CC4)cc3)ccnc2cc1N1CCC(CN2CCN(CC(=O)Nc3c(F)ccc4c3CN([C@H]3CCCNC3=O)C4=O)CC2)CC1]

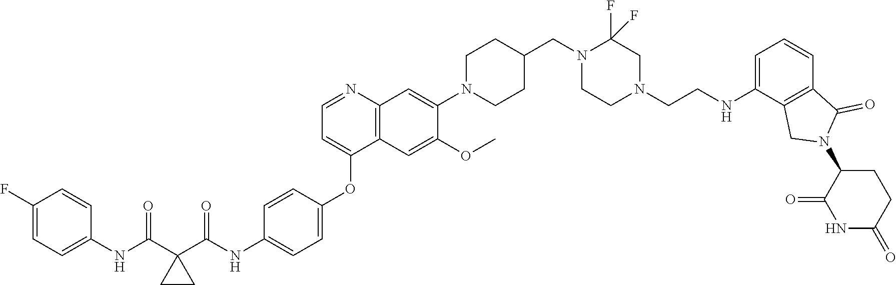 [SMILES: COc1cc2c(Oc3ccc(NC(=O)C4(C(=O)Nc5ccc(F)cc5)CC4)cc3)ccnc2cc1N1CCC(CN2CCN(CCNc3cccc4c3CN([C@H]3CCC(=O)NC3=O)C4=O)CC2(F)F)CC1]

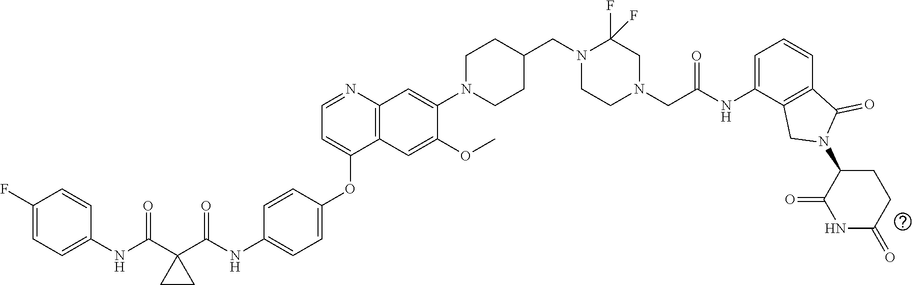 [SMILES: COc1cc2c(Oc3ccc(NC(=O)C4(C(=O)Nc5ccc(F)cc5)CC4)cc3)ccnc2cc1N1CCC(CN2CCN(CC(=O)Nc3cccc4c3CN([C@H]3CCC(=O)NC3=O)C4=O)CC2(F)F)CC1]

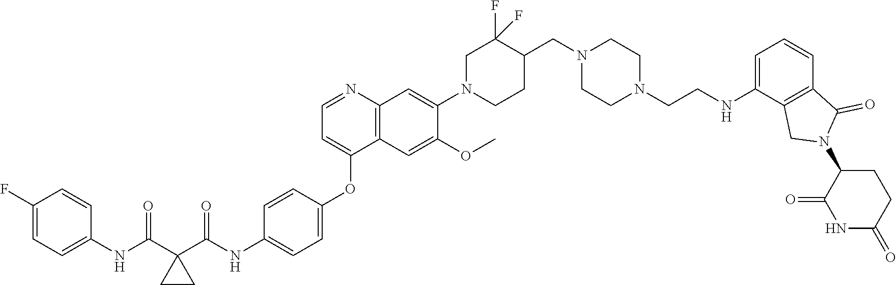 [SMILES: COc1cc2c(Oc3ccc(NC(=O)C4(C(=O)Nc5ccc(F)cc5)CC4)cc3)ccnc2cc1N1CCC(CN2CCN(CCNc3cccc4c3CN([C@H]3CCC(=O)NC3=O)C4=O)CC2)C(F)(F)C1]

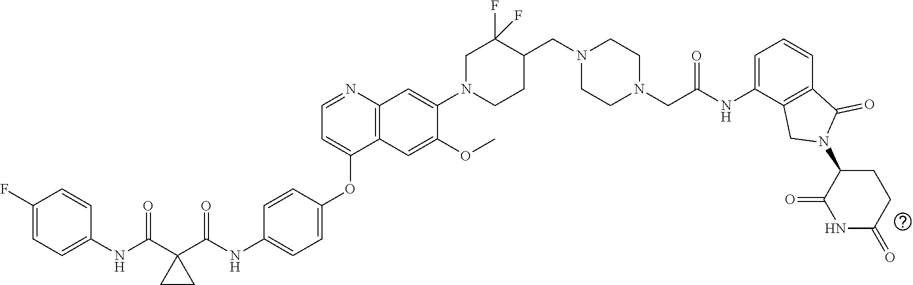 [SMILES: COc1cc2c(Oc3ccc(NC(=O)C4(C(=O)Nc5ccc(F)cc5)CC4)cc3)ccnc2cc1N1CCC(CN2CCN(CC(=O)Nc3cccc4c3CN([C@H]3CCC(=O)NC3=O)C4=O)CC2)C(F)(F)C1]

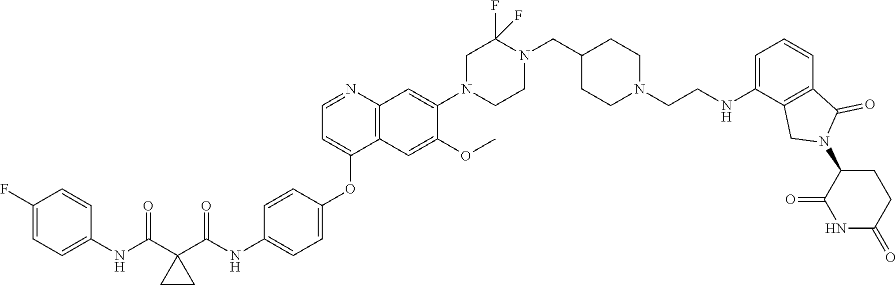 [SMILES: COc1cc2c(Oc3ccc(NC(=O)C4(C(=O)Nc5ccc(F)cc5)CC4)cc3)ccnc2cc1N1CCN(CC2CCN(CCNc3cccc4c3CN([C@H]3CCC(=O)NC3=O)C4=O)CC2)C(F)(F)C1]

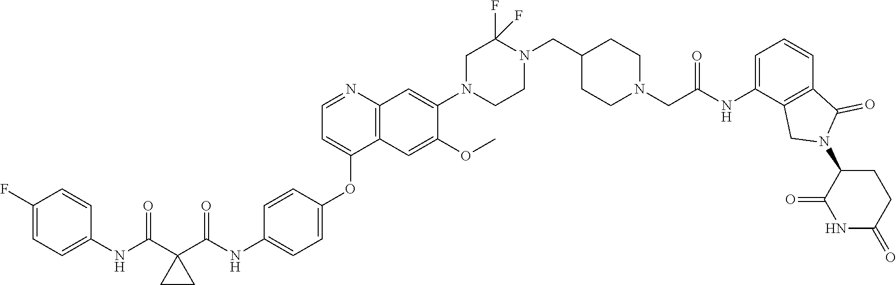 [SMILES: COc1cc2c(Oc3ccc(NC(=O)C4(C(=O)Nc5ccc(F)cc5)CC4)cc3)ccnc2cc1N1CCN(CC2CCN(CC(=O)Nc3cccc4c3CN([C@H]3CCC(=O)NC3=O)C4=O)CC2)C(F)(F)C1]

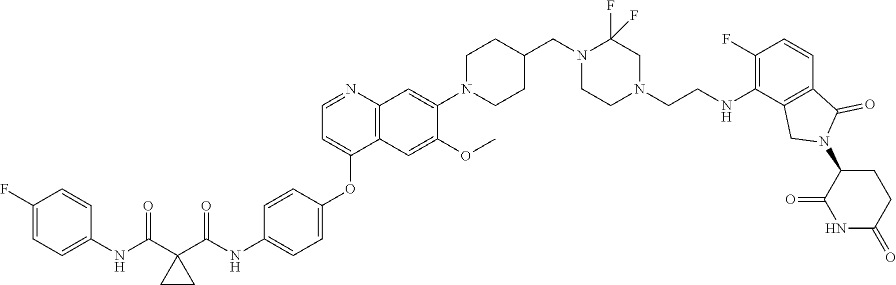 [SMILES: COc1cc2c(Oc3ccc(NC(=O)C4(C(=O)Nc5ccc(F)cc5)CC4)cc3)ccnc2cc1N1CCC(CN2CCN(CCNc3c(F)ccc4c3CN([C@H]3CCC(=O)NC3=O)C4=O)CC2(F)F)CC1]

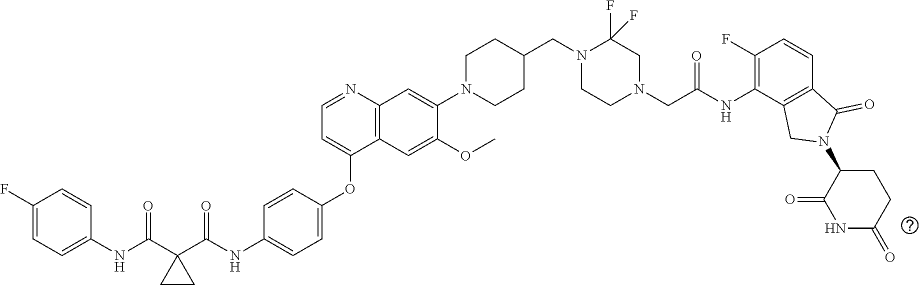 [SMILES: COc1cc2c(Oc3ccc(NC(=O)C4(C(=O)Nc5ccc(F)cc5)CC4)cc3)ccnc2cc1N1CCC(CN2CCN(CC(=O)Nc3c(F)ccc4c3CN([C@H]3CCC(=O)NC3=O)C4=O)CC2(F)F)CC1]

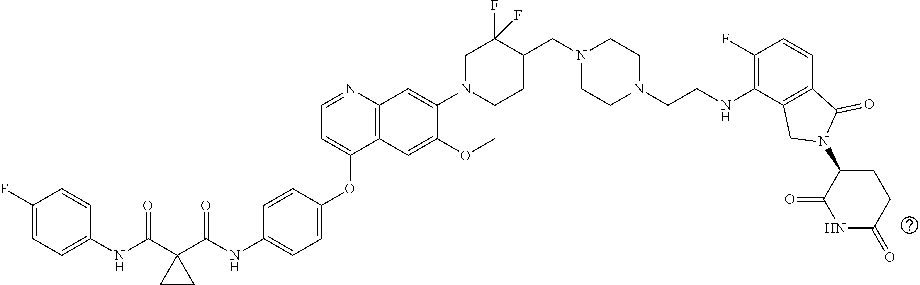 [SMILES: COc1cc2c(Oc3ccc(NC(=O)C4(C(=O)Nc5ccc(F)cc5)CC4)cc3)ccnc2cc1N1CCC(CN2CCN(CCNc3c(F)ccc4c3CN([C@H]3CCC(=O)NC3=O)C4=O)CC2)C(F)(F)C1]

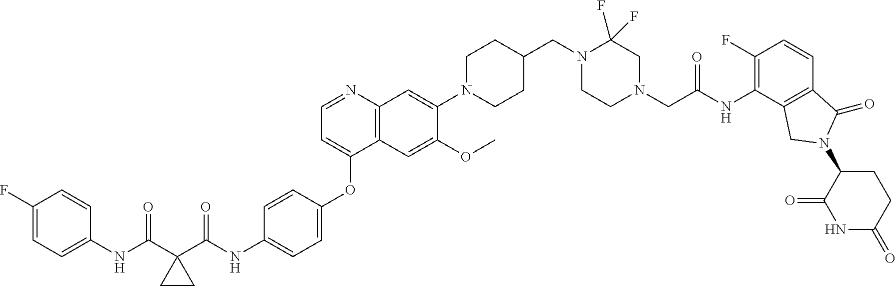 [SMILES: COc1cc2c(Oc3ccc(NC(=O)C4(C(=O)Nc5ccc(F)cc5)CC4)cc3)ccnc2cc1N1CCC(CN2CCN(CC(=O)Nc3c(F)ccc4c3CN([C@H]3CCC(=O)NC3=O)C4=O)CC2(F)F)CC1]

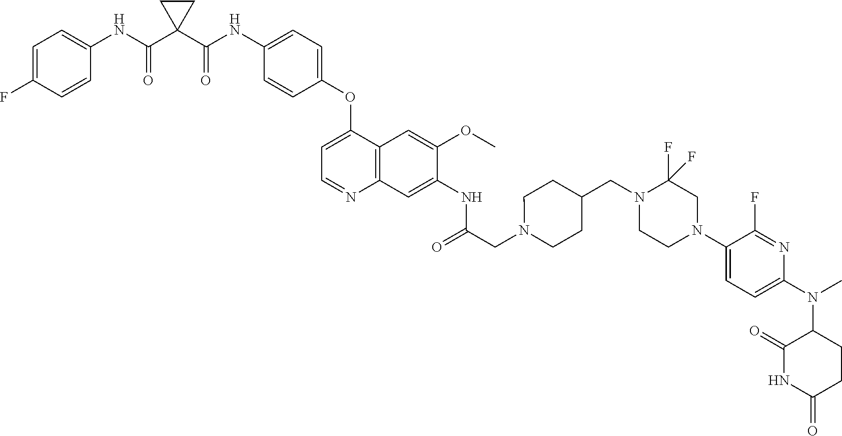 [SMILES: COc1cc2c(Oc3ccc(NC(=O)C4(C(=O)Nc5ccc(F)cc5)CC4)cc3)ccnc2cc1NC(=O)CN1CCC(CN2CCN(c3ccc(N(C)C4CCC(=O)NC4=O)nc3F)CC2(F)F)CC1]

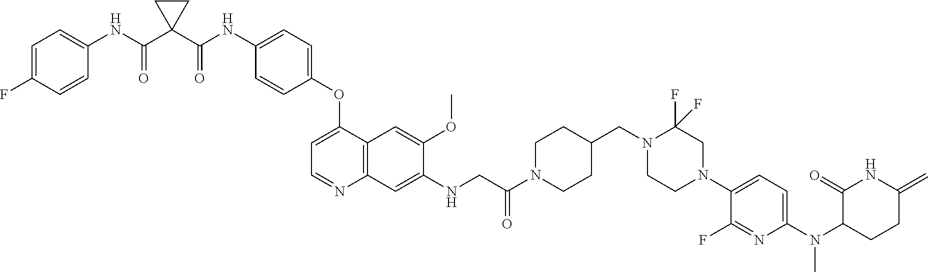 [SMILES: C=C1CCC(N(C)c2ccc(N3CCN(CC4CCN(C(=O)CNc5cc6nccc(Oc7ccc(NC(=O)C8(C(=O)Nc9ccc(F)cc9)CC8)cc7)c6cc5OC)CC4)C(F)(F)C3)c(F)n2)C(=O)N1]

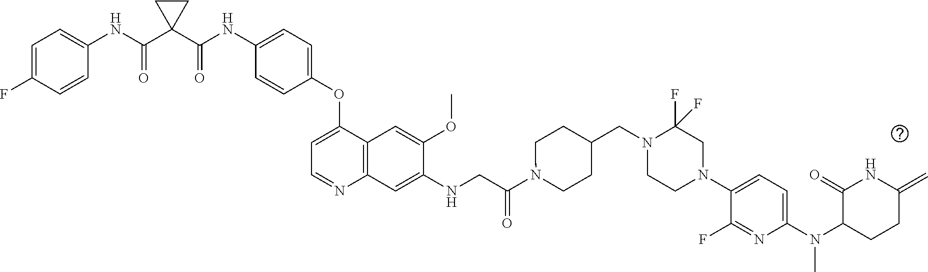 [SMILES: C=C1CCC(N(C)c2ccc(N3CCN(CC4CCN(C(=O)CNc5cc6nccc(Oc7ccc(NC(=O)C8(C(=O)Nc9ccc(F)cc9)CC8)cc7)c6cc5OC)CC4)C(F)(F)C3)c(F)n2)C(=O)N1]

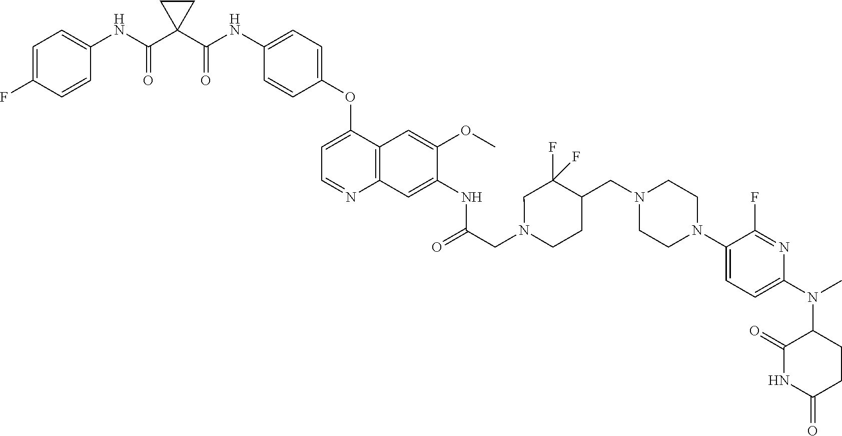 [SMILES: COc1cc2c(Oc3ccc(NC(=O)C4(C(=O)Nc5ccc(F)cc5)CC4)cc3)ccnc2cc1NC(=O)CN1CCC(CN2CCN(c3ccc(N(C)C4CCC(=O)NC4=O)nc3F)CC2)C(F)(F)C1]

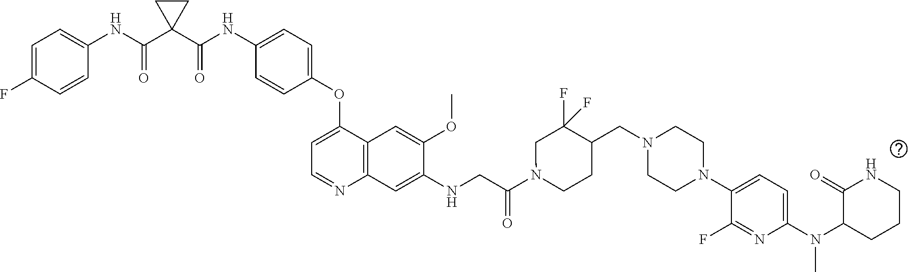 [SMILES: COc1cc2c(Oc3ccc(NC(=O)C4(C(=O)Nc5ccc(F)cc5)CC4)cc3)ccnc2cc1NCC(=O)N1CCC(CN2CCN(c3ccc(N(C)C4CCCNC4=O)nc3F)CC2)C(F)(F)C1]

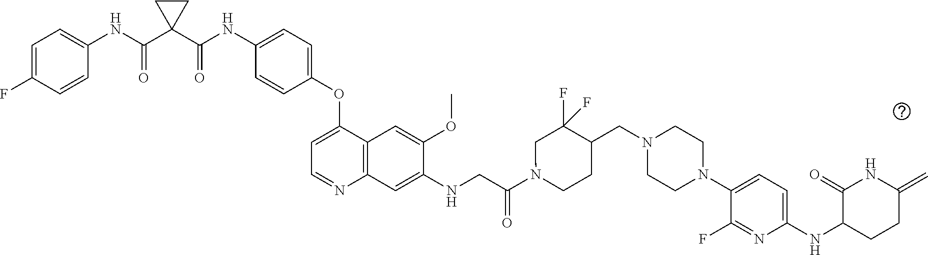 [SMILES: C=C1CCC(Nc2ccc(N3CCN(CC4CCN(C(=O)CNc5cc6nccc(Oc7ccc(NC(=O)C8(C(=O)Nc9ccc(F)cc9)CC8)cc7)c6cc5OC)CC4(F)F)CC3)c(F)n2)C(=O)N1]

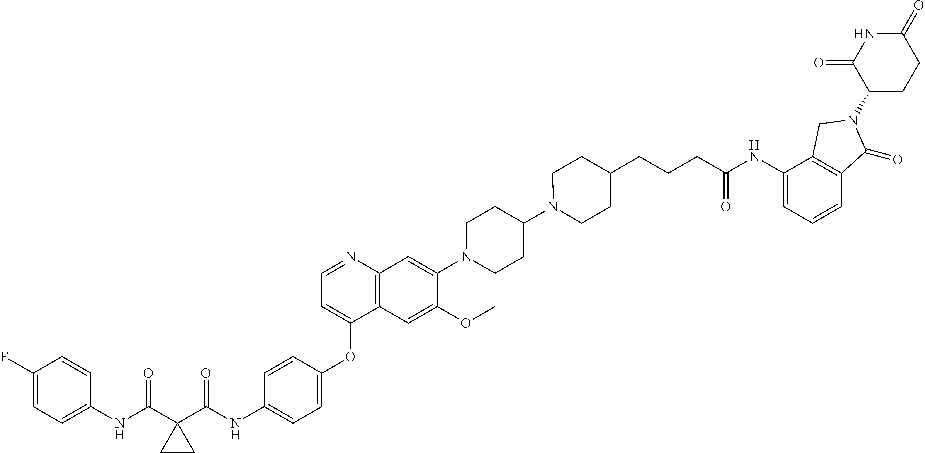 [SMILES: COc1cc2c(Oc3ccc(NC(=O)C4(C(=O)Nc5ccc(F)cc5)CC4)cc3)ccnc2cc1N1CCC(N2CCC(CCCC(=O)Nc3cccc4c3CN([C@H]3CCC(=O)NC3=O)C4=O)CC2)CC1]

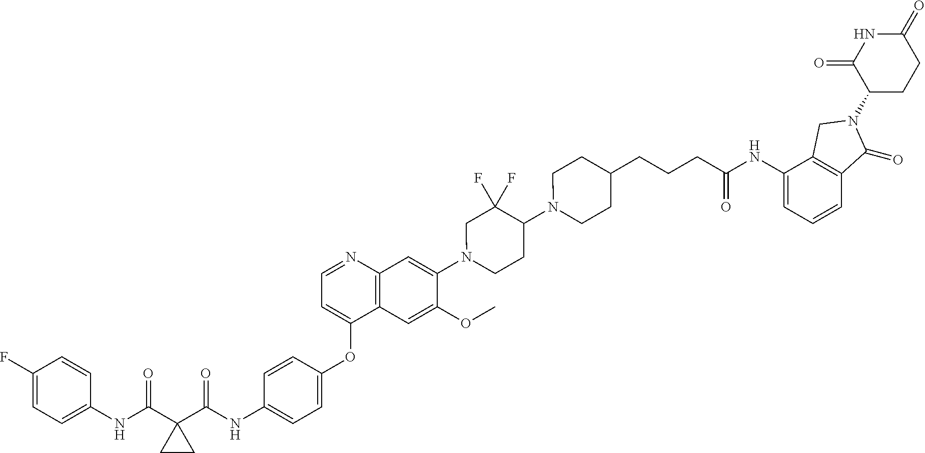 [SMILES: COc1cc2c(Oc3ccc(NC(=O)C4(C(=O)Nc5ccc(F)cc5)CC4)cc3)ccnc2cc1N1CCC(N2CCC(CCCC(=O)Nc3cccc4c3CN([C@H]3CCC(=O)NC3=O)C4=O)CC2)C(F)(F)C1]

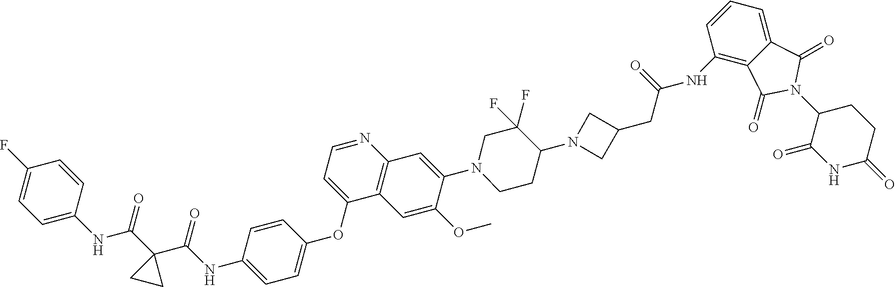 [SMILES: COc1cc2c(Oc3ccc(NC(=O)C4(C(=O)Nc5ccc(F)cc5)CC4)cc3)ccnc2cc1N1CCC(N2CC(CC(=O)Nc3cccc4c3C(=O)N(C3CCC(=O)NC3=O)C4=O)C2)C(F)(F)C1]

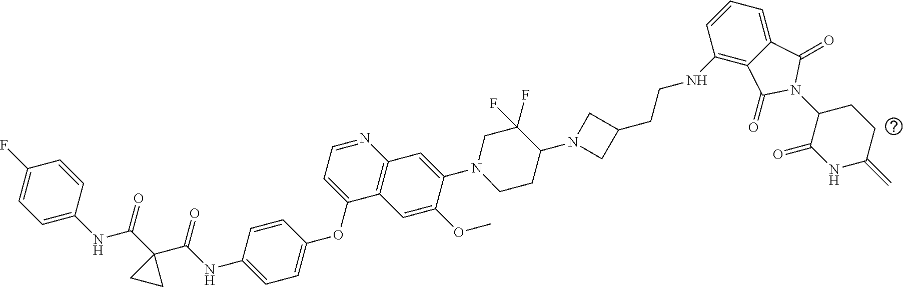 [SMILES: C=C1CCC(N2C(=O)c3cccc(NCCC4CN(C5CCN(c6cc7nccc(Oc8ccc(NC(=O)C9(C(=O)Nc%10ccc(F)cc%10)CC9)cc8)c7cc6OC)CC5(F)F)C4)c3C2=O)C(=O)N1]

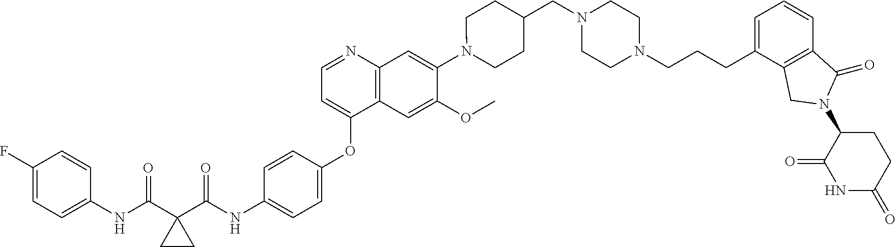 [SMILES: COc1cc2c(Oc3ccc(NC(=O)C4(C(=O)Nc5ccc(F)cc5)CC4)cc3)ccnc2cc1N1CCC(CN2CCN(CCCc3cccc4c3CN([C@H]3CCC(=O)NC3=O)C4=O)CC2)CC1]

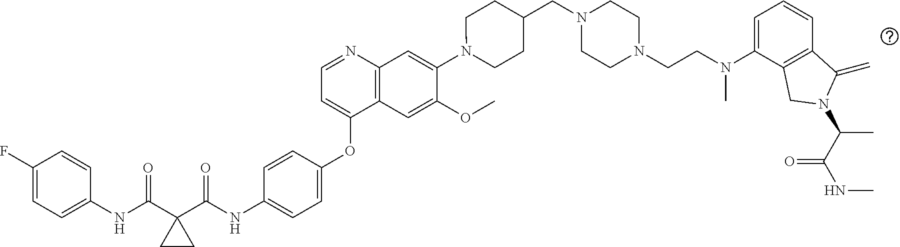 [SMILES: C=C1c2cccc(N(C)CCN3CCN(CC4CCN(c5cc6nccc(Oc7ccc(NC(=O)C8(C(=O)Nc9ccc(F)cc9)CC8)cc7)c6cc5OC)CC4)CC3)c2CN1[C@@H](C)C(=O)NC]

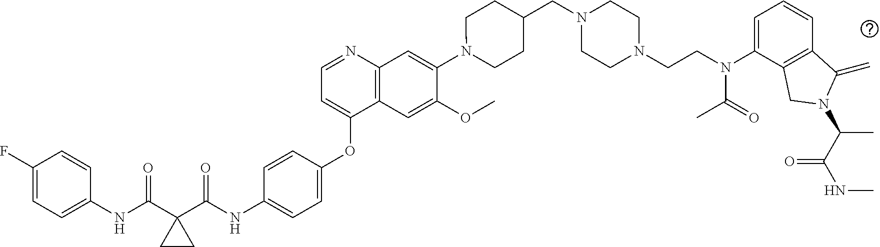 [SMILES: C=C1c2cccc(N(CCN3CCN(CC4CCN(c5cc6nccc(Oc7ccc(NC(=O)C8(C(=O)Nc9ccc(F)cc9)CC8)cc7)c6cc5OC)CC4)CC3)C(C)=O)c2CN1[C@@H](C)C(=O)NC]

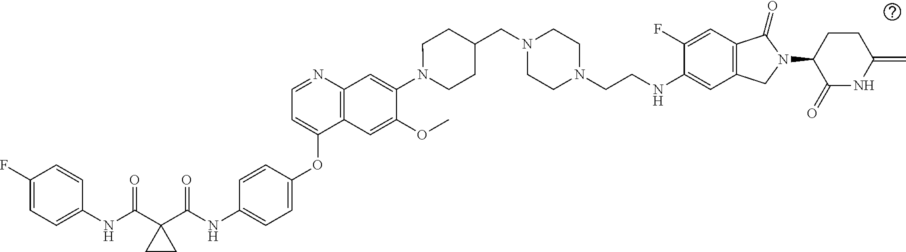 [SMILES: C=C1CC[C@H](N2Cc3cc(NCCN4CCN(CC5CCN(c6cc7nccc(Oc8ccc(NC(=O)C9(C(=O)Nc%10ccc(F)cc%10)CC9)cc8)c7cc6OC)CC5)CC4)c(F)cc3C2=O)C(=O)N1]

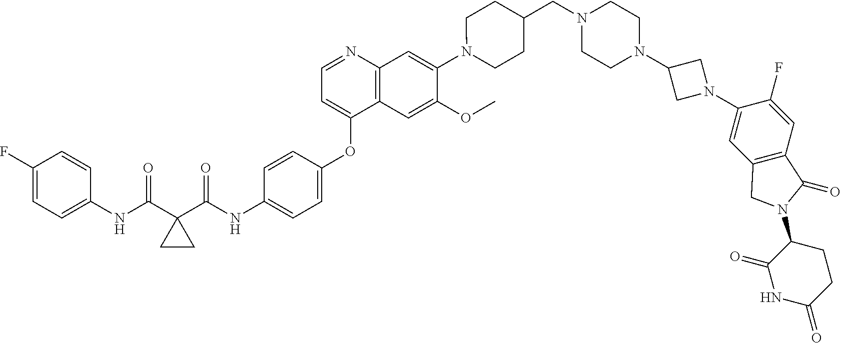 [SMILES: COc1cc2c(Oc3ccc(NC(=O)C4(C(=O)Nc5ccc(F)cc5)CC4)cc3)ccnc2cc1N1CCC(CN2CCN(C3CN(c4cc5c(cc4F)C(=O)N([C@H]4CCC(=O)NC4=O)C5)C3)CC2)CC1]

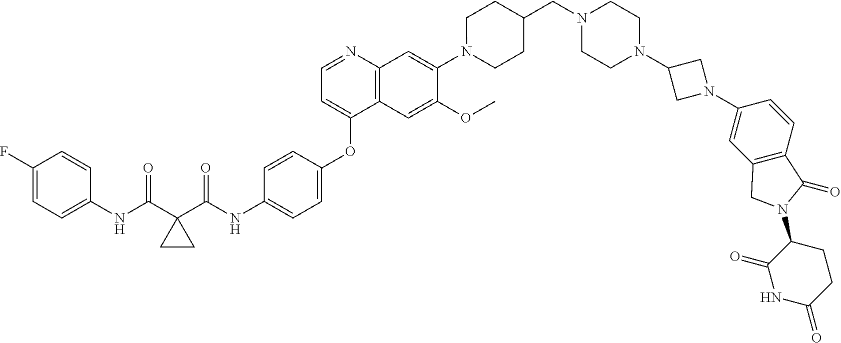 [SMILES: COc1cc2c(Oc3ccc(NC(=O)C4(C(=O)Nc5ccc(F)cc5)CC4)cc3)ccnc2cc1N1CCC(CN2CCN(C3CN(c4ccc5c(c4)CN([C@H]4CCC(=O)NC4=O)C5=O)C3)CC2)CC1]

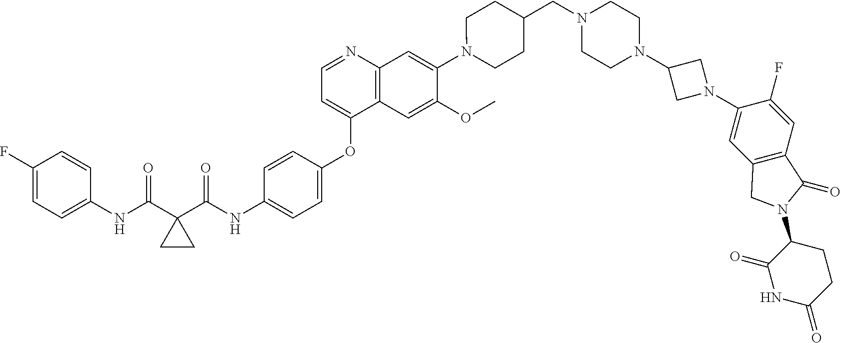 [SMILES: COc1cc2c(Oc3ccc(NC(=O)C4(C(=O)Nc5ccc(F)cc5)CC4)cc3)ccnc2cc1N1CCC(CN2CCN(C3CN(c4cc5c(cc4F)C(=O)N([C@H]4CCC(=O)NC4=O)C5)C3)CC2)CC1]

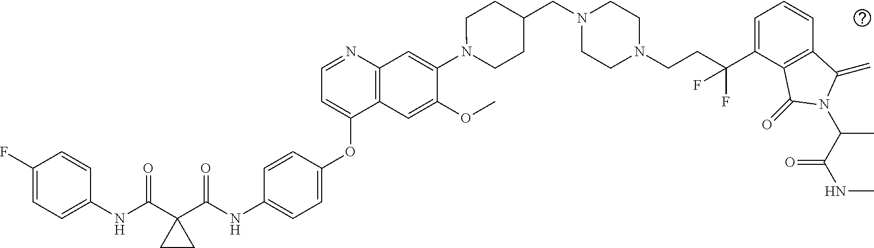 [SMILES: C=C1c2cccc(C(F)(F)CCN3CCN(CC4CCN(c5cc6nccc(Oc7ccc(NC(=O)C8(C(=O)Nc9ccc(F)cc9)CC8)cc7)c6cc5OC)CC4)CC3)c2C(=O)N1C(C)C(=O)NC]

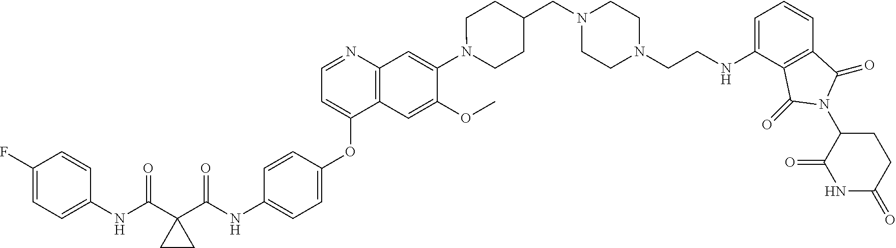 [SMILES: COc1cc2c(Oc3ccc(NC(=O)C4(C(=O)Nc5ccc(F)cc5)CC4)cc3)ccnc2cc1N1CCC(CN2CCN(CCNc3cccc4c3C(=O)N(C3CCC(=O)NC3=O)C4=O)CC2)CC1]

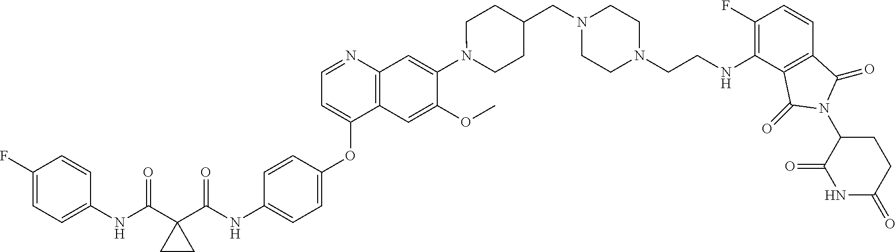 [SMILES: COc1cc2c(Oc3ccc(NC(=O)C4(C(=O)Nc5ccc(F)cc5)CC4)cc3)ccnc2cc1N1CCC(CN2CCN(CCNc3c(F)ccc4c3C(=O)N(C3CCC(=O)NC3=O)C4=O)CC2)CC1]

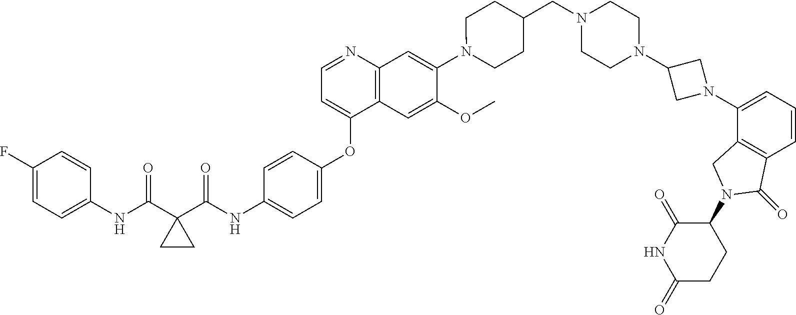 [SMILES: COc1cc2c(Oc3ccc(NC(=O)C4(C(=O)Nc5ccc(F)cc5)CC4)cc3)ccnc2cc1N1CCC(CN2CCN(C3CN(c4cccc5c4CN([C@H]4CCC(=O)NC4=O)C5=O)C3)CC2)CC1]

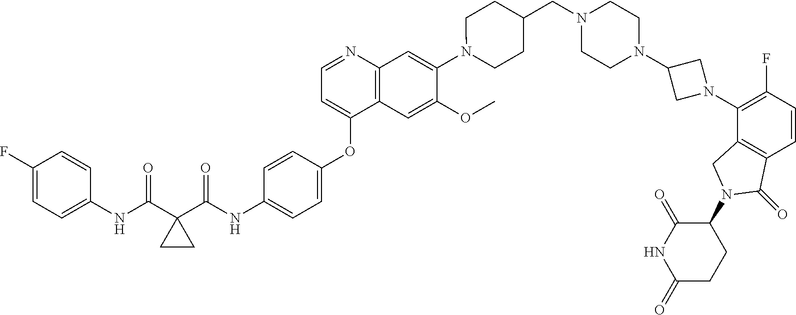 [SMILES: COc1cc2c(Oc3ccc(NC(=O)C4(C(=O)Nc5ccc(F)cc5)CC4)cc3)ccnc2cc1N1CCC(CN2CCN(C3CN(c4c(F)ccc5c4CN([C@H]4CCC(=O)NC4=O)C5=O)C3)CC2)CC1]

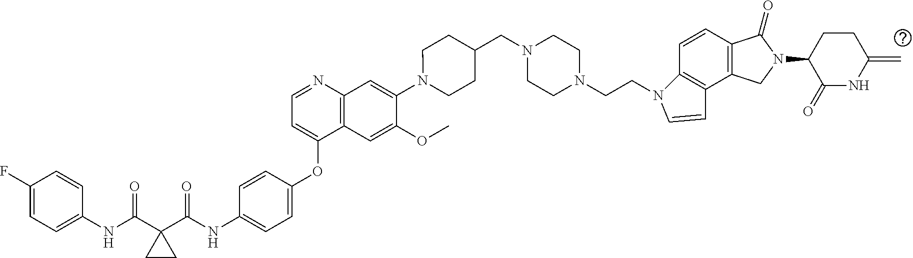 [SMILES: C=C1CC[C@H](N2Cc3c(ccc4c3ccn4CCN3CCN(CC4CCN(c5cc6nccc(Oc7ccc(NC(=O)C8(C(=O)Nc9ccc(F)cc9)CC8)cc7)c6cc5OC)CC4)CC3)C2=O)C(=O)N1]

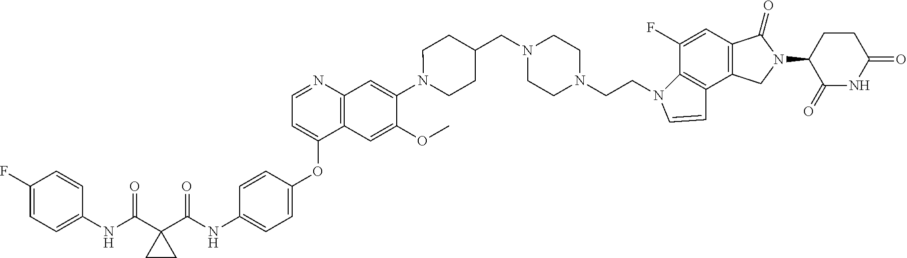 [SMILES: COc1cc2c(Oc3ccc(NC(=O)C4(C(=O)Nc5ccc(F)cc5)CC4)cc3)ccnc2cc1N1CCC(CN2CCN(CCn3ccc4c5c(cc(F)c43)C(=O)N([C@H]3CCC(=O)NC3=O)C5)CC2)CC1]

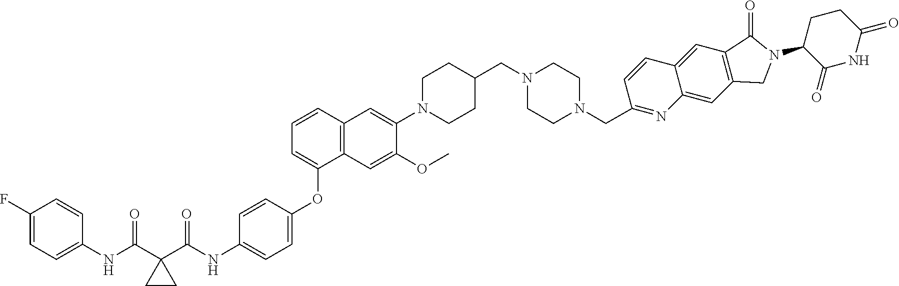 [SMILES: COc1cc2c(Oc3ccc(NC(=O)C4(C(=O)Nc5ccc(F)cc5)CC4)cc3)cccc2cc1N1CCC(CN2CCN(Cc3ccc4cc5c(cc4n3)CN([C@H]3CCC(=O)NC3=O)C5=O)CC2)CC1]

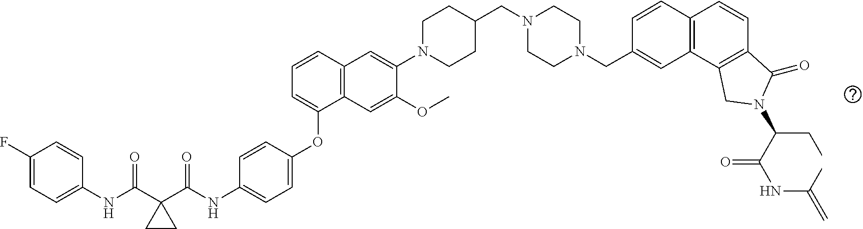 [SMILES: C=C1CC[C@H](N2Cc3c(ccc4ccc(CN5CCN(CC6CCN(c7cc8cccc(Oc9ccc(NC(=O)C%10(C(=O)Nc%11ccc(F)cc%11)CC%10)cc9)c8cc7OC)CC6)CC5)cc34)C2=O)C(=O)N1]

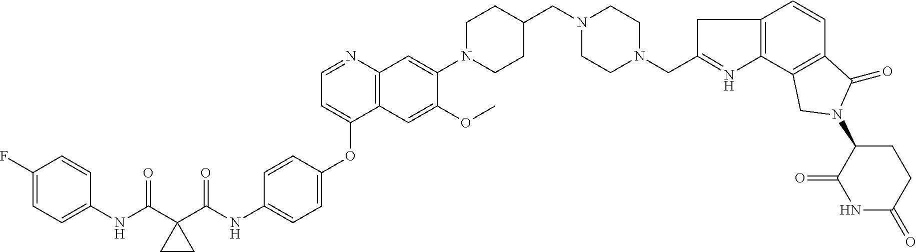 [SMILES: COc1cc2c(Oc3ccc(NC(=O)C4(C(=O)Nc5ccc(F)cc5)CC4)cc3)ccnc2cc1N1CCC(CN2CCN(CC3=Nc4c(ccc5c4CN([C@H]4CCC(=O)NC4=O)C5=O)C3)CC2)CC1]

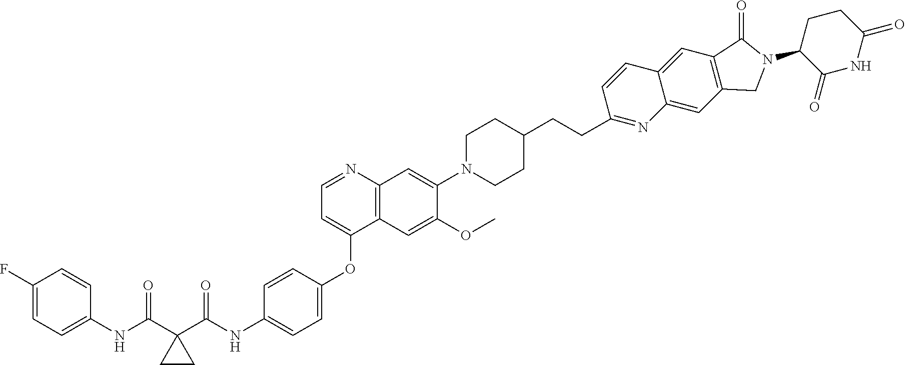 [SMILES: COc1cc2c(Oc3ccc(NC(=O)C4(C(=O)Nc5ccc(F)cc5)CC4)cc3)ccnc2cc1N1CCC(CCc2ccc3cc4c(cc3n2)CN([C@H]2CCC(=O)NC2=O)C4=O)CC1]

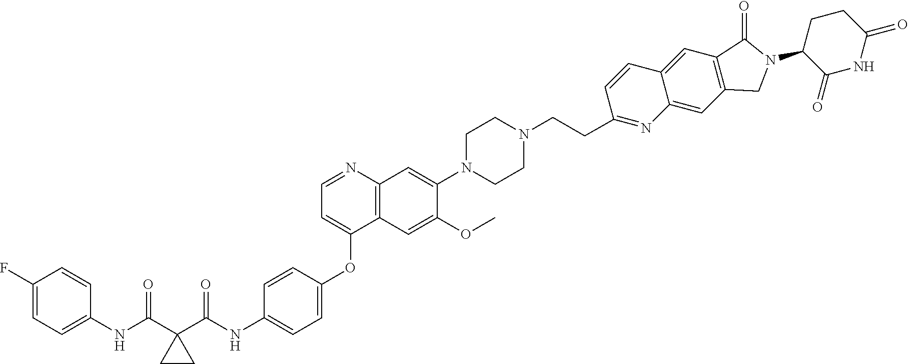 [SMILES: COc1cc2c(Oc3ccc(NC(=O)C4(C(=O)Nc5ccc(F)cc5)CC4)cc3)ccnc2cc1N1CCN(CCc2ccc3cc4c(cc3n2)CN([C@H]2CCC(=O)NC2=O)C4=O)CC1]

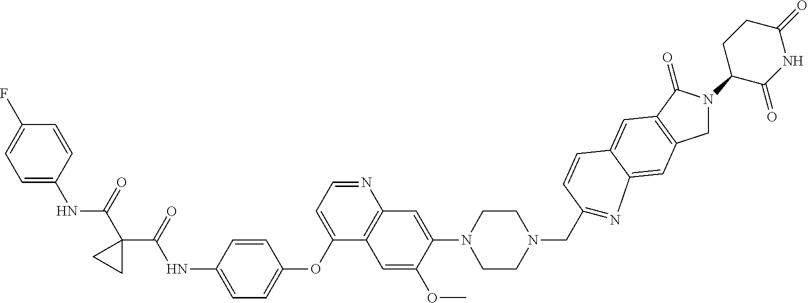 [SMILES: COc1cc2c(Oc3ccc(NC(=O)C4(C(=O)Nc5ccc(F)cc5)CC4)cc3)ccnc2cc1N1CCN(Cc2ccc3cc4c(cc3n2)CN([C@H]2CCC(=O)NC2=O)C4=O)CC1]

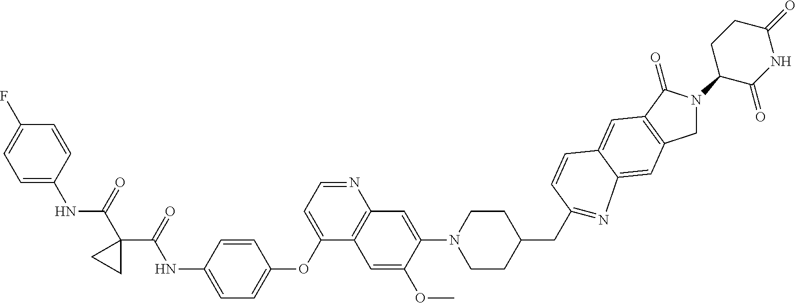 [SMILES: COc1cc2c(Oc3ccc(NC(=O)C4(C(=O)Nc5ccc(F)cc5)CC4)cc3)ccnc2cc1N1CCC(Cc2ccc3cc4c(cc3n2)CN([C@H]2CCC(=O)NC2=O)C4=O)CC1]

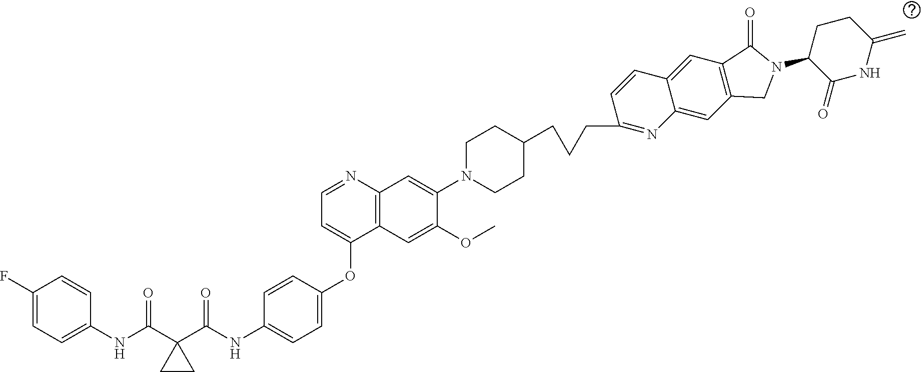 [SMILES: C=C1CC[C@H](N2Cc3cc4nc(CCCC5CCN(c6cc7nccc(Oc8ccc(NC(=O)C9(C(=O)Nc%10ccc(F)cc%10)CC9)cc8)c7cc6OC)CC5)ccc4cc3C2=O)C(=O)N1]

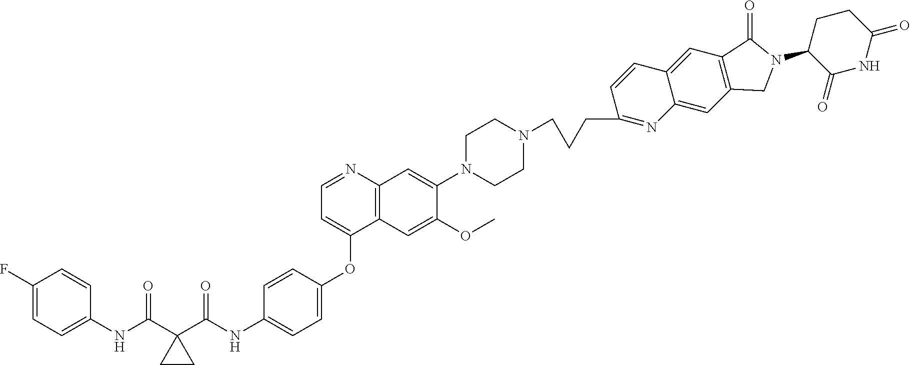 [SMILES: COc1cc2c(Oc3ccc(NC(=O)C4(C(=O)Nc5ccc(F)cc5)CC4)cc3)ccnc2cc1N1CCN(CCCc2ccc3cc4c(cc3n2)CN([C@H]2CCC(=O)NC2=O)C4=O)CC1]

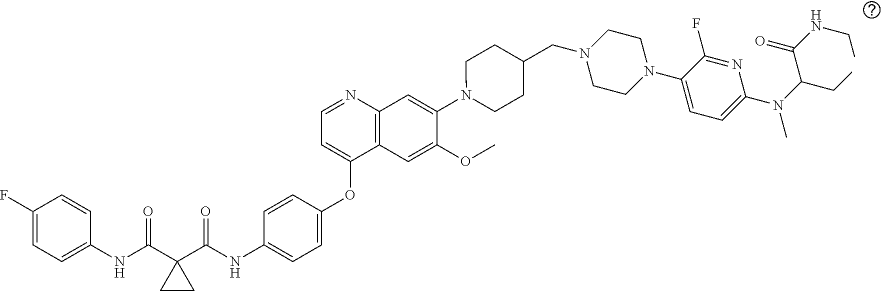 [SMILES: COc1cc2c(Oc3ccc(NC(=O)C4(C(=O)Nc5ccc(F)cc5)CC4)cc3)ccnc2cc1N1CCC(CN2CCN(c3ccc(N(C)C4CCCNC4=O)nc3F)CC2)CC1]

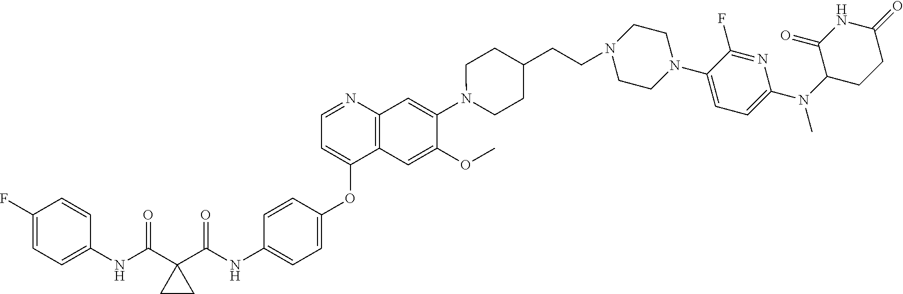 [SMILES: COc1cc2c(Oc3ccc(NC(=O)C4(C(=O)Nc5ccc(F)cc5)CC4)cc3)ccnc2cc1N1CCC(CCN2CCN(c3ccc(N(C)C4CCC(=O)NC4=O)nc3F)CC2)CC1]

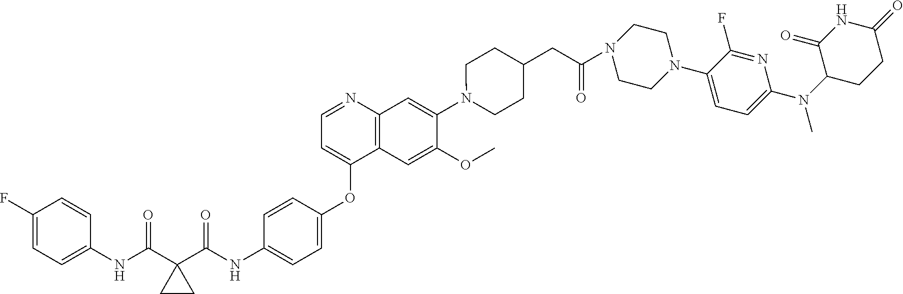 [SMILES: COc1cc2c(Oc3ccc(NC(=O)C4(C(=O)Nc5ccc(F)cc5)CC4)cc3)ccnc2cc1N1CCC(CC(=O)N2CCN(c3ccc(N(C)C4CCC(=O)NC4=O)nc3F)CC2)CC1]

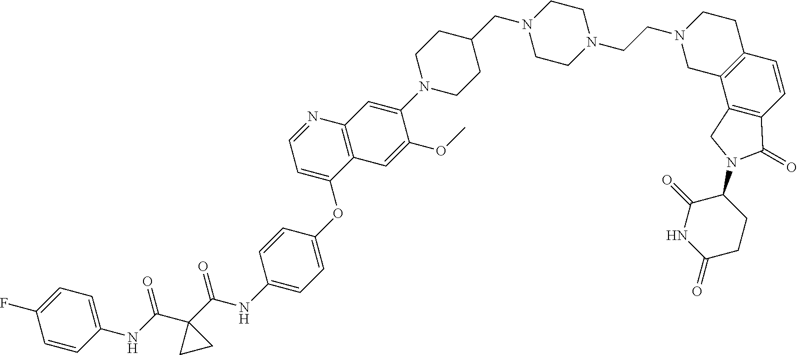 [SMILES: COc1cc2c(Oc3ccc(NC(=O)C4(C(=O)Nc5ccc(F)cc5)CC4)cc3)ccnc2cc1N1CCC(CN2CCN(CCN3CCc4ccc5c(c4C3)CN([C@H]3CCC(=O)NC3=O)C5=O)CC2)CC1]

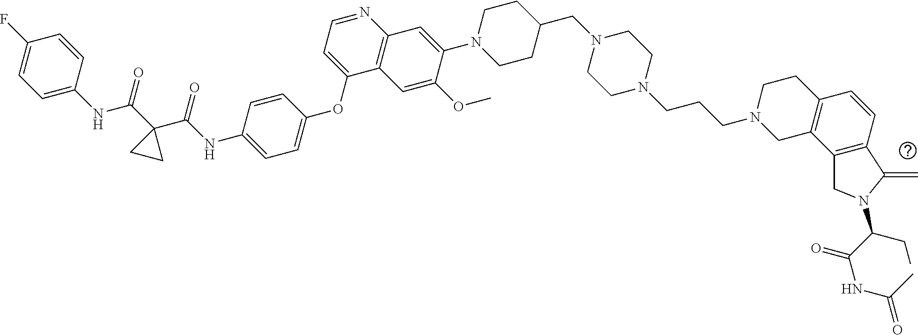 [SMILES: C=C1c2ccc3c(c2CN1[C@@H](CC)C(=O)NC(C)=O)CN(CCCN1CCN(CC2CCN(c4cc5nccc(Oc6ccc(NC(=O)C7(C(=O)Nc8ccc(F)cc8)CC7)cc6)c5cc4OC)CC2)CC1)CC3]

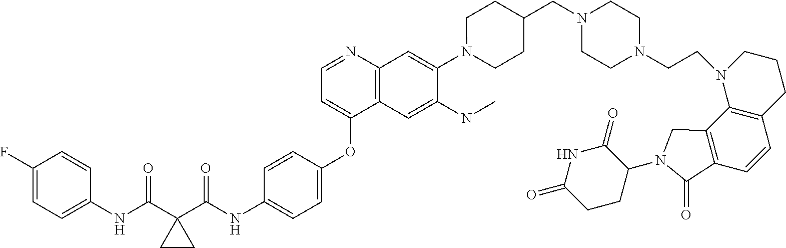 [SMILES: CNc1cc2c(Oc3ccc(NC(=O)C4(C(=O)Nc5ccc(F)cc5)CC4)cc3)ccnc2cc1N1CCC(CN2CCN(CCN3CCCc4ccc5c(c43)CN(C3CCC(=O)NC3=O)C5=O)CC2)CC1]

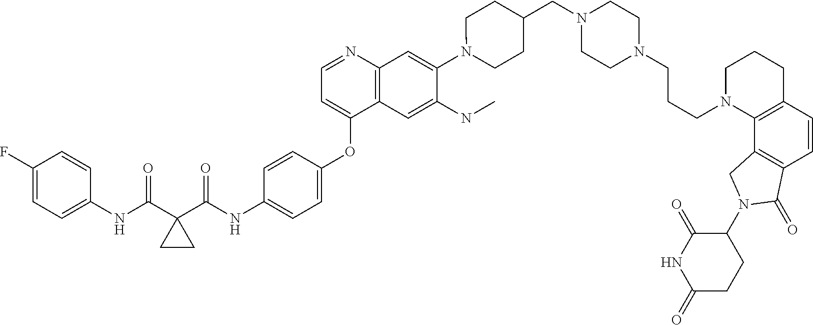 [SMILES: CNc1cc2c(Oc3ccc(NC(=O)C4(C(=O)Nc5ccc(F)cc5)CC4)cc3)ccnc2cc1N1CCC(CN2CCN(CCCN3CCCc4ccc5c(c43)CN(C3CCC(=O)NC3=O)C5=O)CC2)CC1]

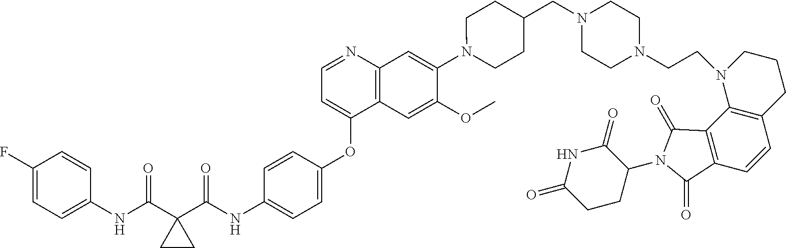 [SMILES: COc1cc2c(Oc3ccc(NC(=O)C4(C(=O)Nc5ccc(F)cc5)CC4)cc3)ccnc2cc1N1CCC(CN2CCN(CCN3CCCc4ccc5c(c43)C(=O)N(C3CCC(=O)NC3=O)C5=O)CC2)CC1]

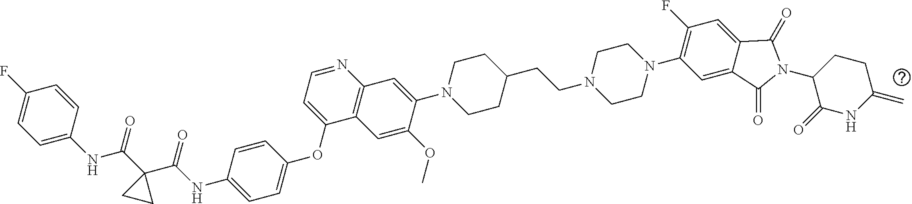 [SMILES: C=C1CCC(N2C(=O)c3cc(F)c(N4CCN(CCC5CCN(c6cc7nccc(Oc8ccc(NC(=O)C9(C(=O)Nc%10ccc(F)cc%10)CC9)cc8)c7cc6OC)CC5)CC4)cc3C2=O)C(=O)N1]

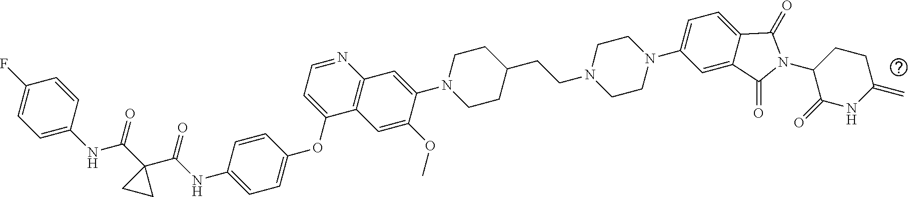 [SMILES: C=C1CCC(N2C(=O)c3ccc(N4CCN(CCC5CCN(c6cc7nccc(Oc8ccc(NC(=O)C9(C(=O)Nc%10ccc(F)cc%10)CC9)cc8)c7cc6OC)CC5)CC4)cc3C2=O)C(=O)N1]

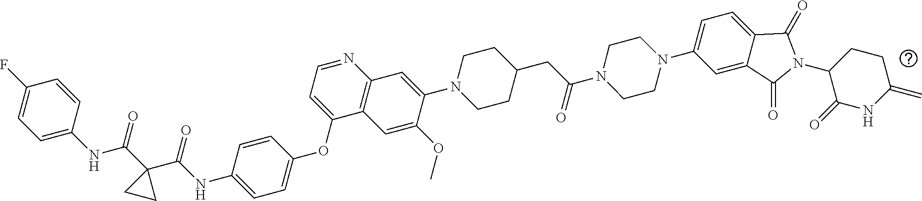 [SMILES: C=C1CCC(N2C(=O)c3ccc(N4CCN(C(=O)CC5CCN(c6cc7nccc(Oc8ccc(NC(=O)C9(C(=O)Nc%10ccc(F)cc%10)CC9)cc8)c7cc6OC)CC5)CC4)cc3C2=O)C(=O)N1]

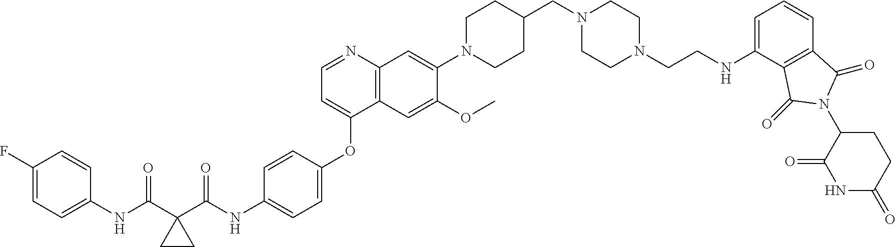 [SMILES: COc1cc2c(Oc3ccc(NC(=O)C4(C(=O)Nc5ccc(F)cc5)CC4)cc3)ccnc2cc1N1CCC(CN2CCN(CCNc3cccc4c3C(=O)N(C3CCC(=O)NC3=O)C4=O)CC2)CC1]

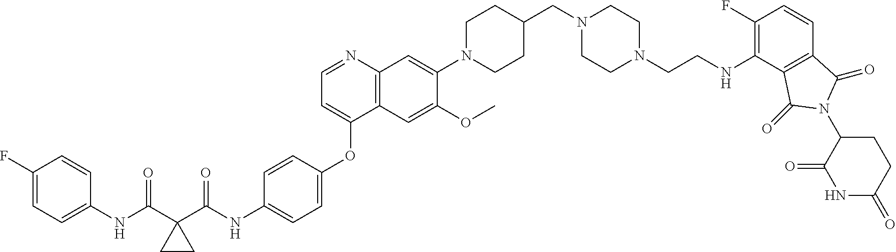 [SMILES: COc1cc2c(Oc3ccc(NC(=O)C4(C(=O)Nc5ccc(F)cc5)CC4)cc3)ccnc2cc1N1CCC(CN2CCN(CCNc3c(F)ccc4c3C(=O)N(C3CCC(=O)NC3=O)C4=O)CC2)CC1]

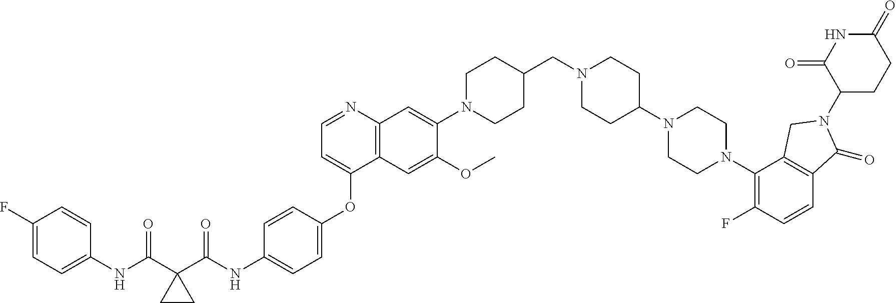 [SMILES: COc1cc2c(Oc3ccc(NC(=O)C4(C(=O)Nc5ccc(F)cc5)CC4)cc3)ccnc2cc1N1CCC(CN2CCC(N3CCN(c4c(F)ccc5c4CN(C4CCC(=O)NC4=O)C5=O)CC3)CC2)CC1]

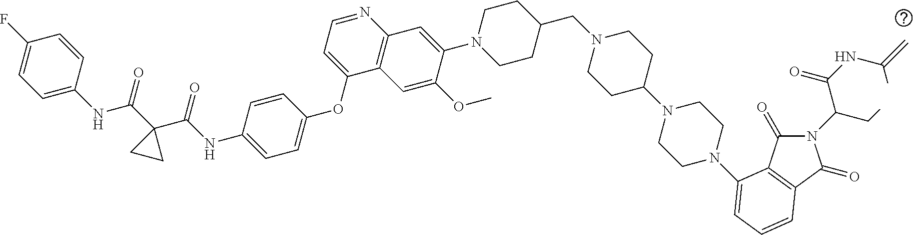 [SMILES: C=C1CCC(N2C(=O)c3cccc(N4CCN(C5CCN(CC6CCN(c7cc8nccc(Oc9ccc(NC(=O)C%10(C(=O)Nc%11ccc(F)cc%11)CC%10)cc9)c8cc7OC)CC6)CC5)CC4)c3C2=O)C(=O)N1]

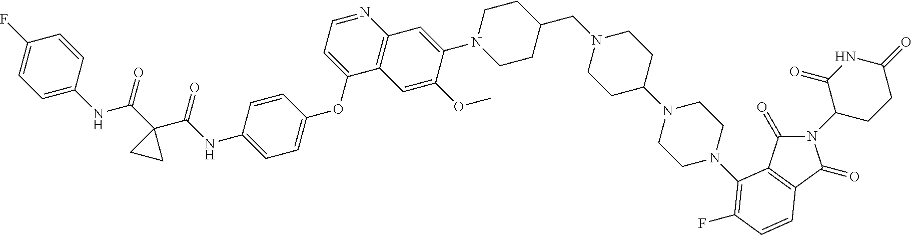 [SMILES: COc1cc2c(Oc3ccc(NC(=O)C4(C(=O)Nc5ccc(F)cc5)CC4)cc3)ccnc2cc1N1CCC(CN2CCC(N3CCN(c4c(F)ccc5c4C(=O)N(C4CCC(=O)NC4=O)C5=O)CC3)CC2)CC1]

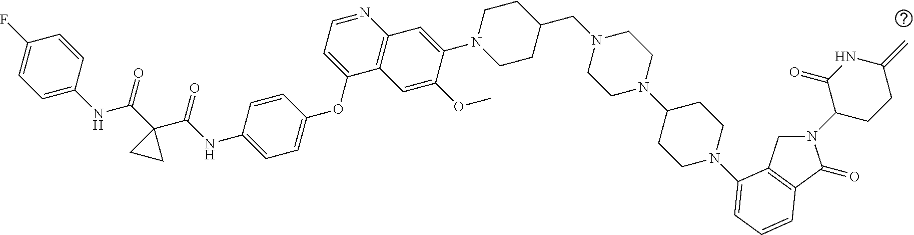 [SMILES: C=C1CCC(N2Cc3c(cccc3N3CCC(N4CCN(CC5CCN(c6cc7nccc(Oc8ccc(NC(=O)C9(C(=O)Nc%10ccc(F)cc%10)CC9)cc8)c7cc6OC)CC5)CC4)CC3)C2=O)C(=O)N1]

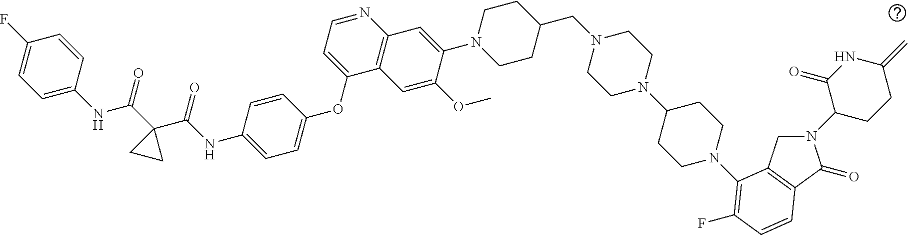 [SMILES: C=C1CCC(N2Cc3c(ccc(F)c3N3CCC(N4CCN(CC5CCN(c6cc7nccc(Oc8ccc(NC(=O)C9(C(=O)Nc%10ccc(F)cc%10)CC9)cc8)c7cc6OC)CC5)CC4)CC3)C2=O)C(=O)N1]

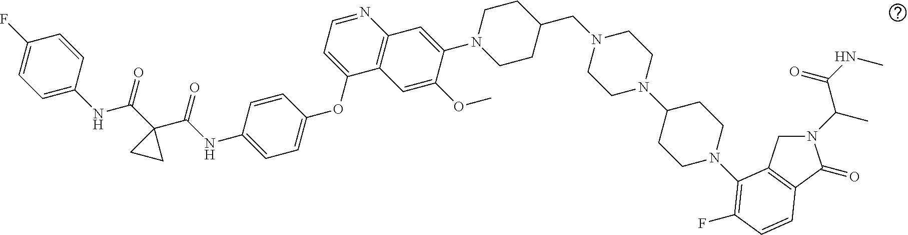 [SMILES: CNC(=O)C(C)N1Cc2c(ccc(F)c2N2CCC(N3CCN(CC4CCN(c5cc6nccc(Oc7ccc(NC(=O)C8(C(=O)Nc9ccc(F)cc9)CC8)cc7)c6cc5OC)CC4)CC3)CC2)C1=O]

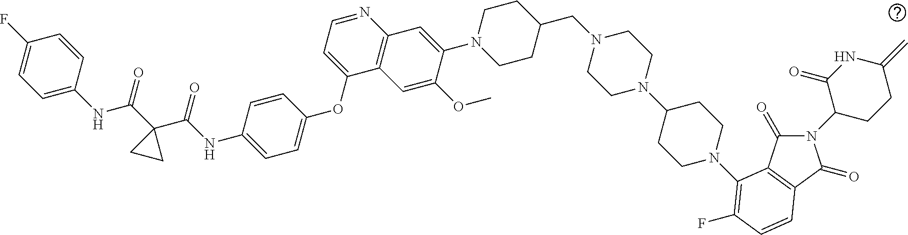 [SMILES: C=C1CCC(N2C(=O)c3ccc(F)c(N4CCC(N5CCN(CC6CCN(c7cc8nccc(Oc9ccc(NC(=O)C%10(C(=O)Nc%11ccc(F)cc%11)CC%10)cc9)c8cc7OC)CC6)CC5)CC4)c3C2=O)C(=O)N1]

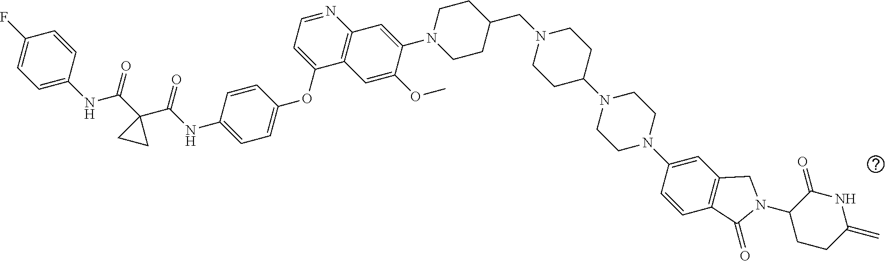 [SMILES: C=C1CCC(N2Cc3cc(N4CCN(C5CCN(CC6CCN(c7cc8nccc(Oc9ccc(NC(=O)C%10(C(=O)Nc%11ccc(F)cc%11)CC%10)cc9)c8cc7OC)CC6)CC5)CC4)ccc3C2=O)C(=O)N1]

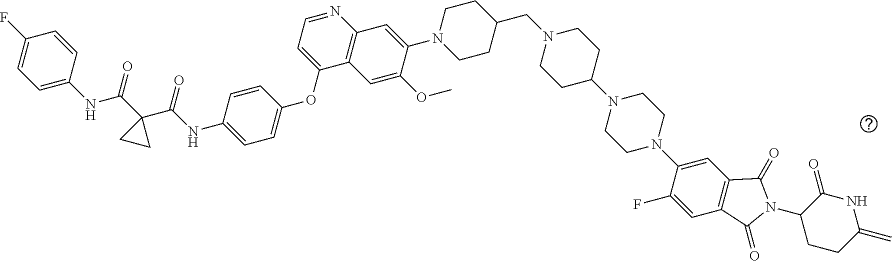 [SMILES: C=C1CCC(N2C(=O)c3cc(F)c(N4CCN(C5CCN(CC6CCN(c7cc8nccc(Oc9ccc(NC(=O)C%10(C(=O)Nc%11ccc(F)cc%11)CC%10)cc9)c8cc7OC)CC6)CC5)CC4)cc3C2=O)C(=O)N1]

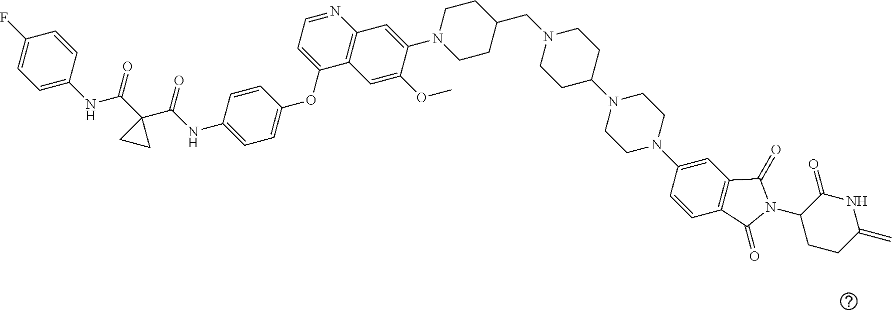 [SMILES: C=C1CCC(N2C(=O)c3ccc(N4CCN(C5CCN(CC6CCN(c7cc8nccc(Oc9ccc(NC(=O)C%10(C(=O)Nc%11ccc(F)cc%11)CC%10)cc9)c8cc7OC)CC6)CC5)CC4)cc3C2=O)C(=O)N1]

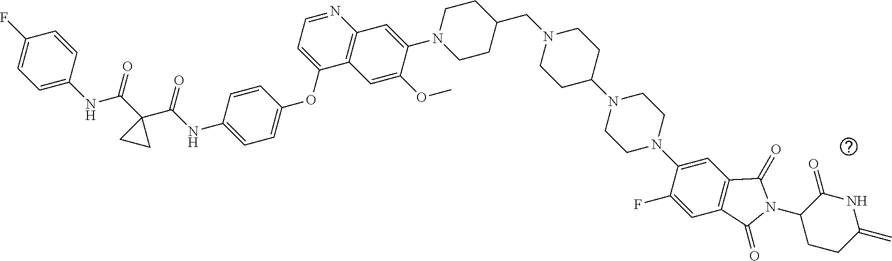 [SMILES: C=C1CCC(N2C(=O)c3cc(F)c(N4CCN(C5CCN(CC6CCN(c7cc8nccc(Oc9ccc(NC(=O)C%10(C(=O)Nc%11ccc(F)cc%11)CC%10)cc9)c8cc7OC)CC6)CC5)CC4)cc3C2=O)C(=O)N1]

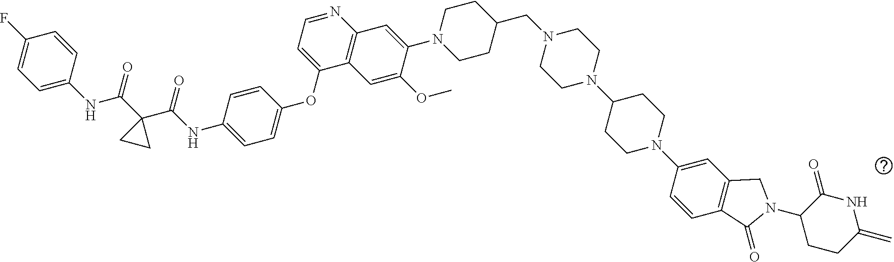 [SMILES: C=C1CCC(N2Cc3cc(N4CCC(N5CCN(CC6CCN(c7cc8nccc(Oc9ccc(NC(=O)C%10(C(=O)Nc%11ccc(F)cc%11)CC%10)cc9)c8cc7OC)CC6)CC5)CC4)ccc3C2=O)C(=O)N1]

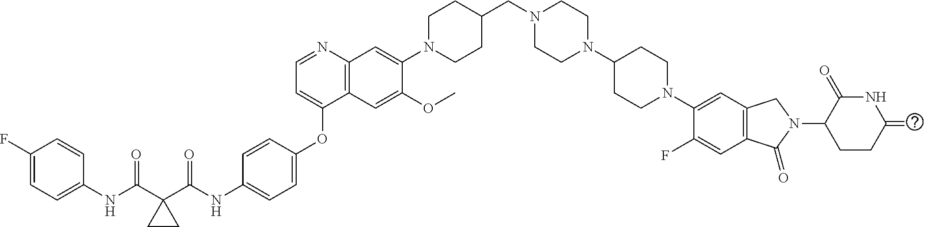 [SMILES: C=C1CCC(N2Cc3cc(N4CCC(N5CCN(CC6CCN(c7cc8nccc(Oc9ccc(NC(=O)C%10(C(=O)Nc%11ccc(F)cc%11)CC%10)cc9)c8cc7OC)CC6)CC5)CC4)c(F)cc3C2=O)C(=O)N1]

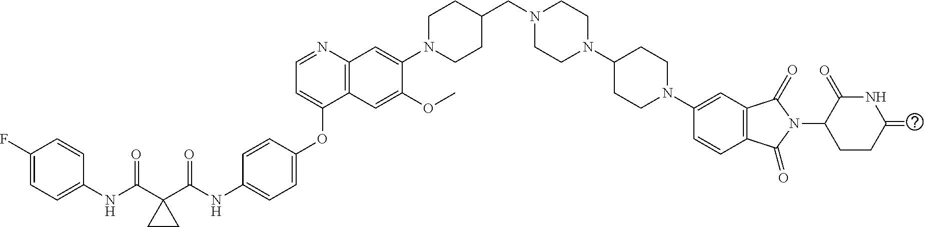 [SMILES: C=C1CCC(N2C(=O)c3ccc(N4CCC(N5CCN(CC6CCN(c7cc8nccc(Oc9ccc(NC(=O)C%10(C(=O)Nc%11ccc(F)cc%11)CC%10)cc9)c8cc7OC)CC6)CC5)CC4)cc3C2=O)C(=O)N1]

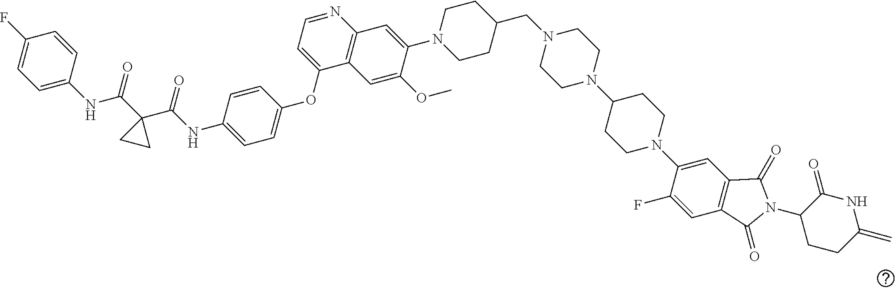 [SMILES: C=C1CCC(N2C(=O)c3cc(F)c(N4CCC(N5CCN(CC6CCN(c7cc8nccc(Oc9ccc(NC(=O)C%10(C(=O)Nc%11ccc(F)cc%11)CC%10)cc9)c8cc7OC)CC6)CC5)CC4)cc3C2=O)C(=O)N1]

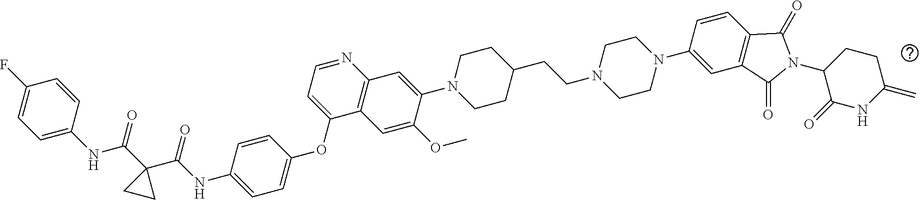 [SMILES: C=C1CCC(N2C(=O)c3ccc(N4CCN(CCC5CCN(c6cc7nccc(Oc8ccc(NC(=O)C9(C(=O)Nc%10ccc(F)cc%10)CC9)cc8)c7cc6OC)CC5)CC4)cc3C2=O)C(=O)N1]

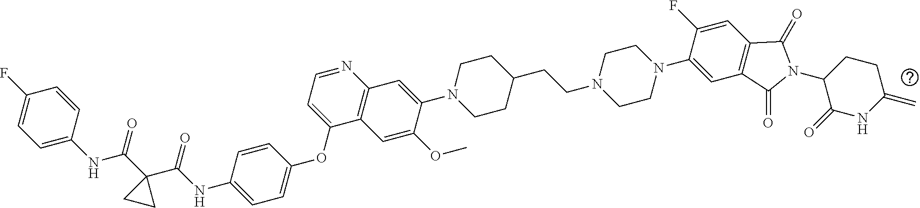 [SMILES: C=C1CCC(N2C(=O)c3cc(F)c(N4CCN(CCC5CCN(c6cc7nccc(Oc8ccc(NC(=O)C9(C(=O)Nc%10ccc(F)cc%10)CC9)cc8)c7cc6OC)CC5)CC4)cc3C2=O)C(=O)N1]

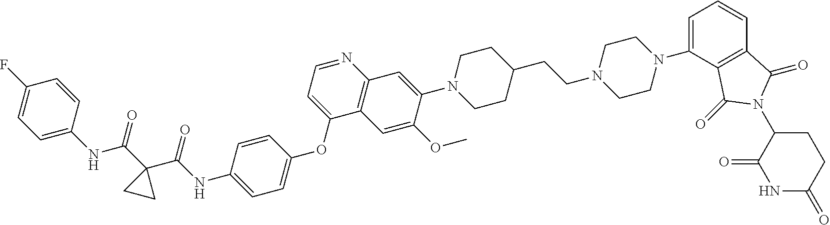 [SMILES: COc1cc2c(Oc3ccc(NC(=O)C4(C(=O)Nc5ccc(F)cc5)CC4)cc3)ccnc2cc1N1CCC(CCN2CCN(c3cccc4c3C(=O)N(C3CCC(=O)NC3=O)C4=O)CC2)CC1]

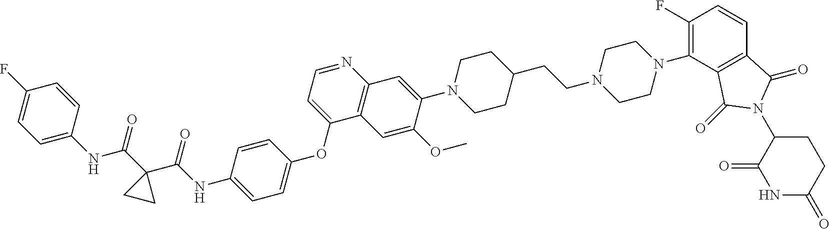 [SMILES: COc1cc2c(Oc3ccc(NC(=O)C4(C(=O)Nc5ccc(F)cc5)CC4)cc3)ccnc2cc1N1CCC(CCN2CCN(c3c(F)ccc4c3C(=O)N(C3CCC(=O)NC3=O)C4=O)CC2)CC1]

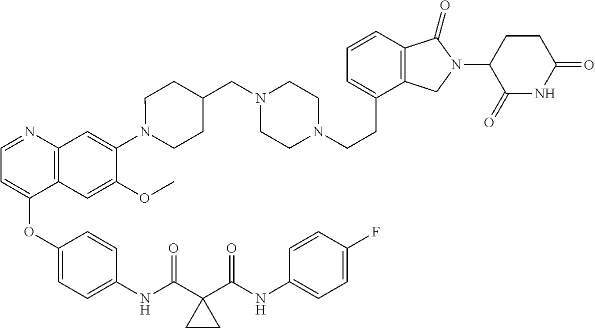 [SMILES: COc1cc2c(Oc3ccc(NC(=O)C4(C(=O)Nc5ccc(F)cc5)CC4)cc3)ccnc2cc1N1CCC(CN2CCN(CCc3cccc4c3CN(C3CCC(=O)NC3=O)C4=O)CC2)CC1]

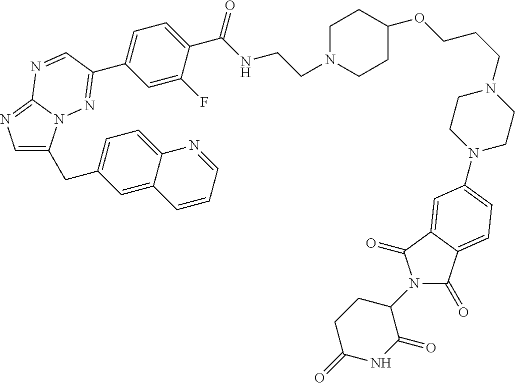 [SMILES: O=C1CCC(N2C(=O)c3ccc(N4CCN(CCCOC5CCN(CCNC(=O)c6ccc(-c7cnc8ncc(Cc9ccc%10ncccc%10c9)n8n7)cc6F)CC5)CC4)cc3C2=O)C(=O)N1]

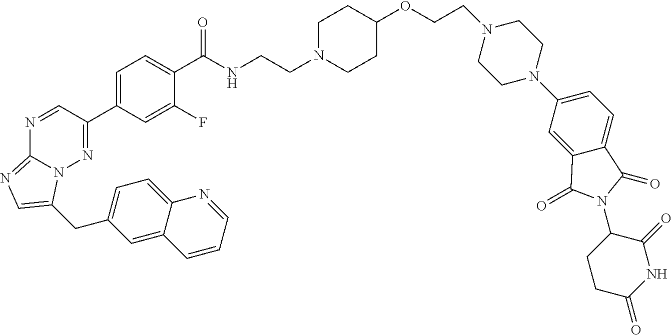 [SMILES: O=C1CCC(N2C(=O)c3ccc(N4CCN(CCOC5CCN(CCNC(=O)c6ccc(-c7cnc8ncc(Cc9ccc%10ncccc%10c9)n8n7)cc6F)CC5)CC4)cc3C2=O)C(=O)N1]

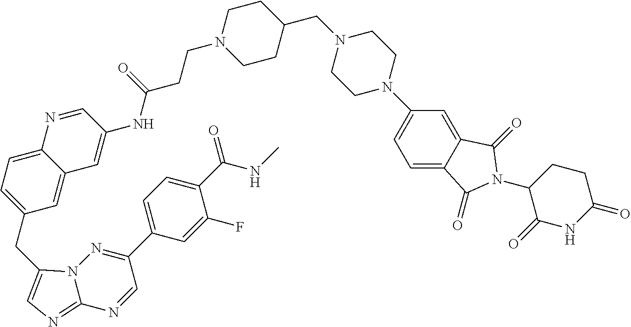 [SMILES: CNC(=O)c1ccc(-c2cnc3ncc(Cc4ccc5ncc(NC(=O)CCN6CCC(CN7CCN(c8ccc9c(c8)C(=O)N(C8CCC(=O)NC8=O)C9=O)CC7)CC6)cc5c4)n3n2)cc1F]

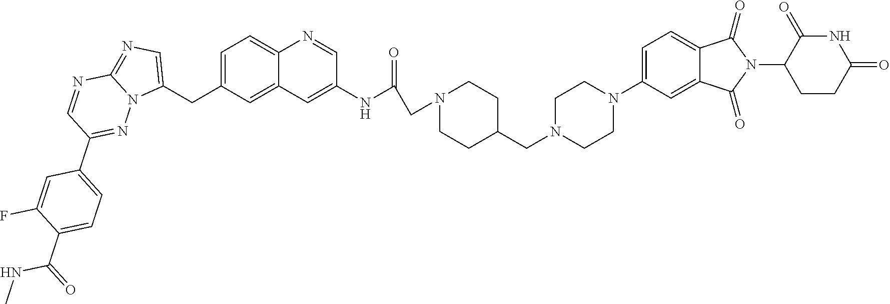 [SMILES: CNC(=O)c1ccc(-c2cnc3ncc(Cc4ccc5ncc(NC(=O)CN6CCC(CN7CCN(c8ccc9c(c8)C(=O)N(C8CCC(=O)NC8=O)C9=O)CC7)CC6)cc5c4)n3n2)cc1F]

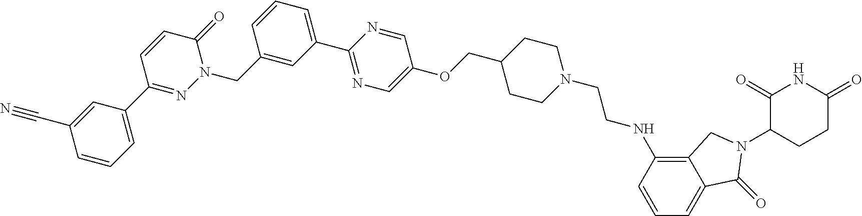 [SMILES: N#Cc1cccc(-c2ccc(=O)n(Cc3cccc(-c4ncc(OCC5CCN(CCNc6cccc7c6CN(C6CCC(=O)NC6=O)C7=O)CC5)cn4)c3)n2)c1]

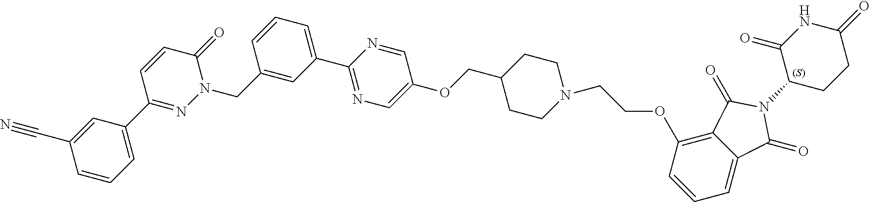

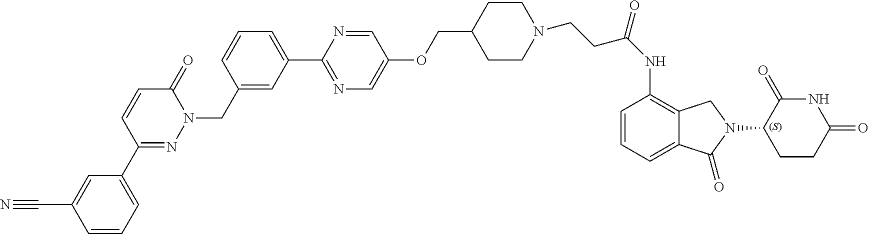

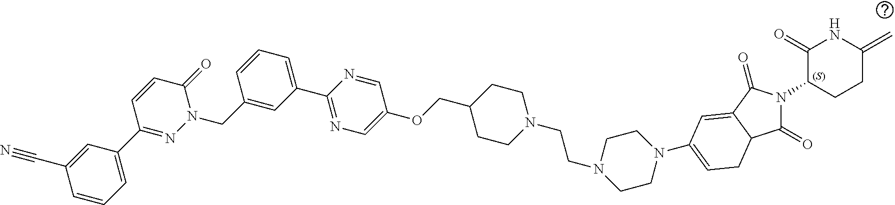

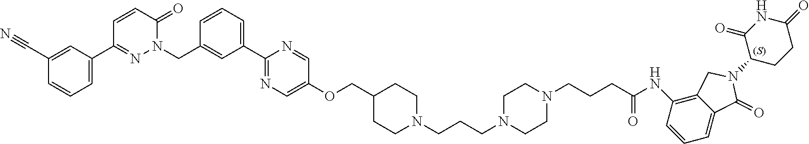

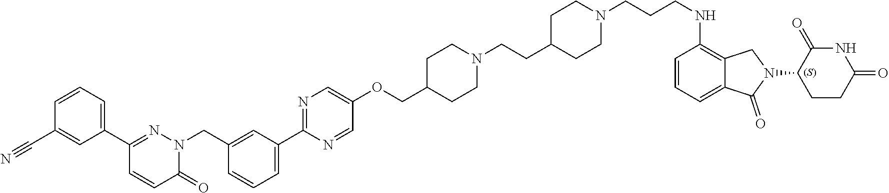

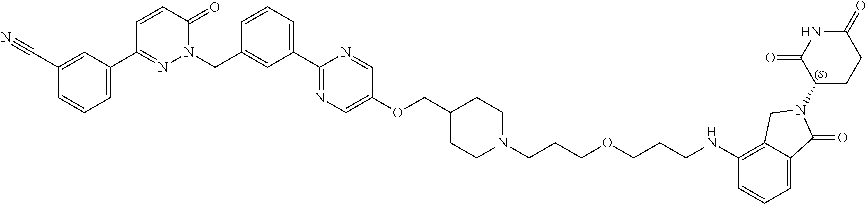

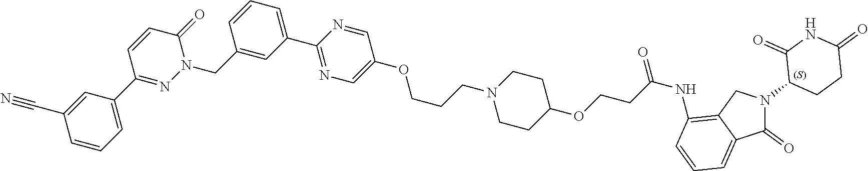

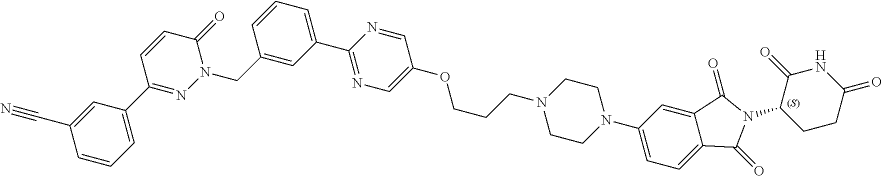

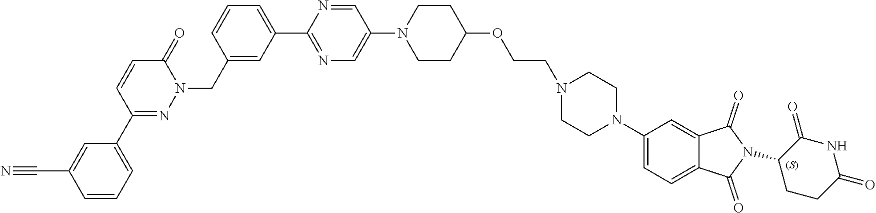

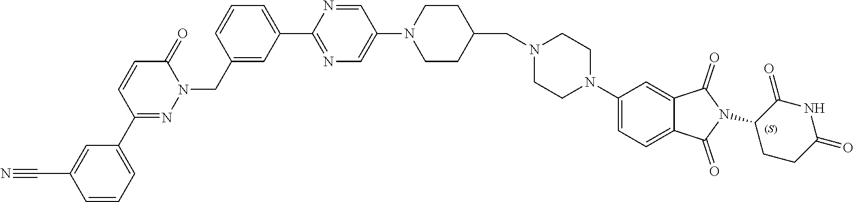

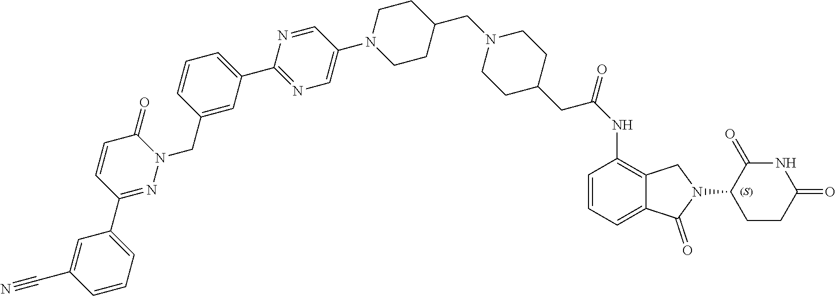

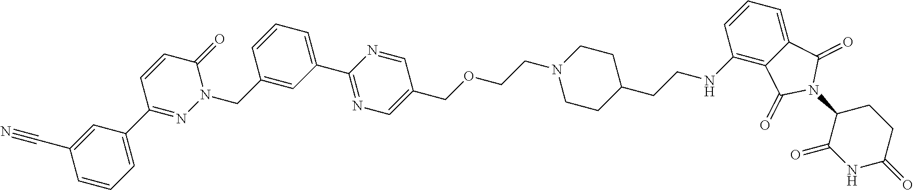 [SMILES: N#Cc1cccc(-c2ccc(=O)n(Cc3cccc(-c4ncc(COCCN5CCC(CCNc6cccc7c6C(=O)N([C@H]6CCC(=O)NC6=O)C7=O)CC5)cn4)c3)n2)c1]

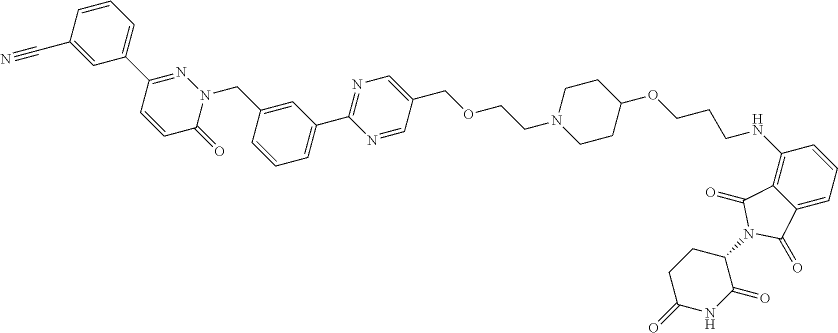 [SMILES: N#Cc1cccc(-c2ccc(=O)n(Cc3cccc(-c4ncc(COCCN5CCC(OCCCNc6cccc7c6C(=O)N([C@H]6CCC(=O)NC6=O)C7=O)CC5)cn4)c3)n2)c1]

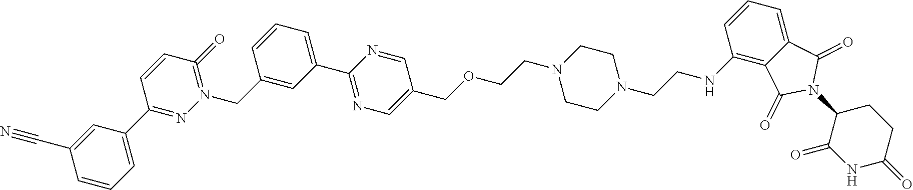

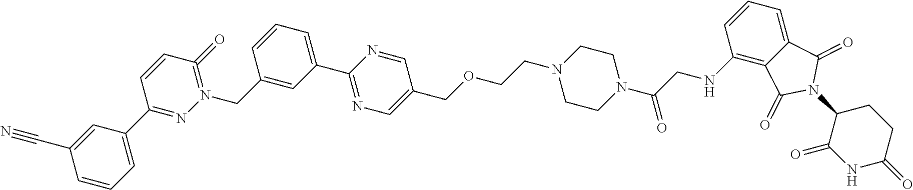

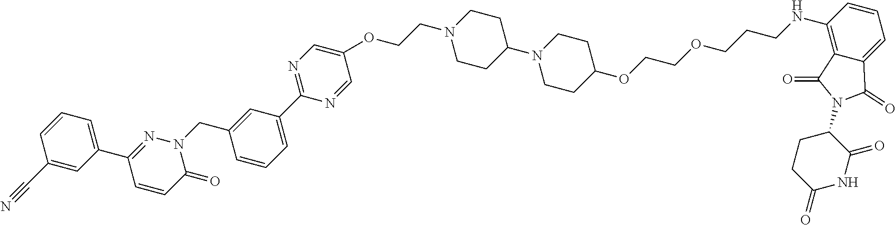 [SMILES: N#Cc1cccc(-c2ccc(=O)n(Cc3cccc(-c4ncc(OCCN5CCC(N6CCC(OCCOCCCNc7cccc8c7C(=O)N([C@H]7CCC(=O)NC7=O)C8=O)CC6)CC5)cn4)c3)n2)c1]

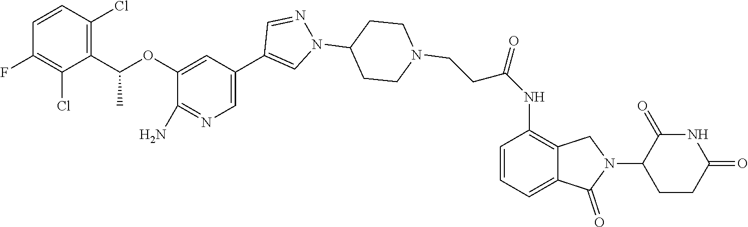 [SMILES: C[C@@H](Oc1cc(-c2cnn(C3CCN(CCC(=O)Nc4cccc5c4CN(C4CCC(=O)NC4=O)C5=O)CC3)c2)cnc1N)c1c(Cl)ccc(F)c1Cl]

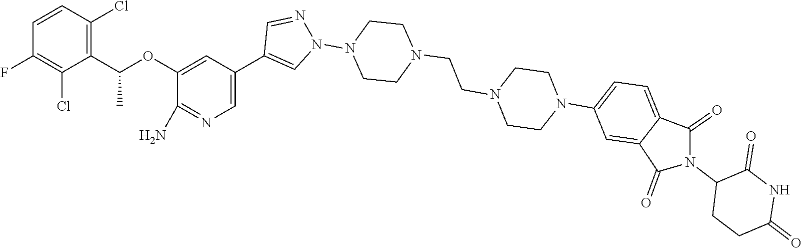 [SMILES: C[C@@H](Oc1cc(-c2cnn(N3CCN(CCN4CCN(c5ccc6c(c5)C(=O)N(C5CCC(=O)NC5=O)C6=O)CC4)CC3)c2)cnc1N)c1c(Cl)ccc(F)c1Cl]

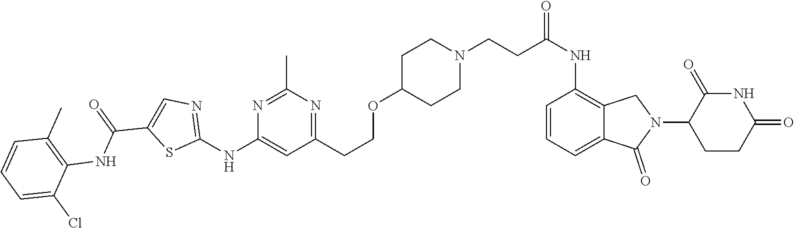 [SMILES: Cc1nc(CCOC2CCN(CCC(=O)Nc3cccc4c3CN(C3CCC(=O)NC3=O)C4=O)CC2)cc(Nc2ncc(C(=O)Nc3c(C)cccc3Cl)s2)n1]

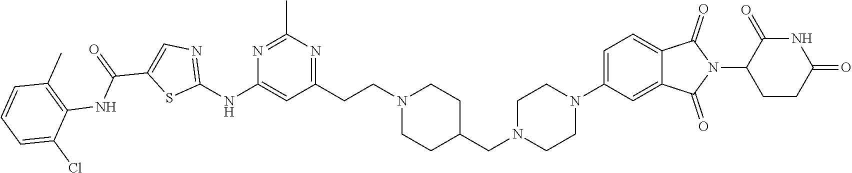 [SMILES: Cc1nc(CCN2CCC(CN3CCN(c4ccc5c(c4)C(=O)N(C4CCC(=O)NC4=O)C5=O)CC3)CC2)cc(Nc2ncc(C(=O)Nc3c(C)cccc3Cl)s2)n1]

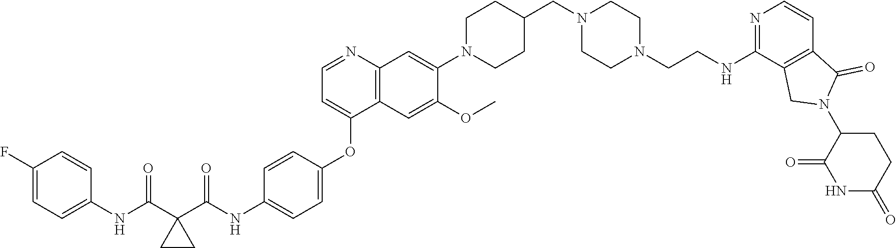 [SMILES: COc1cc2c(Oc3ccc(NC(=O)C4(C(=O)Nc5ccc(F)cc5)CC4)cc3)ccnc2cc1N1CCC(CN2CCN(CCNc3nccc4c3CN(C3CCC(=O)NC3=O)C4=O)CC2)CC1]

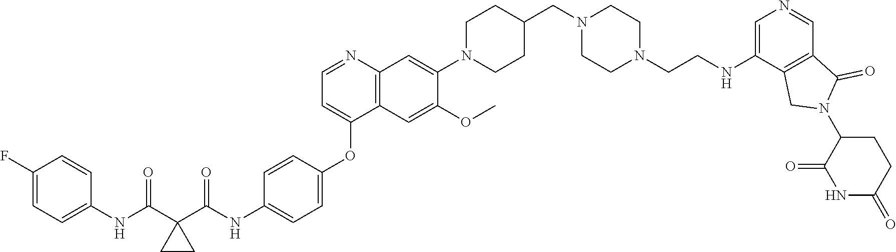 [SMILES: COc1cc2c(Oc3ccc(NC(=O)C4(C(=O)Nc5ccc(F)cc5)CC4)cc3)ccnc2cc1N1CCC(CN2CCN(CCNc3cncc4c3CN(C3CCC(=O)NC3=O)C4=O)CC2)CC1]

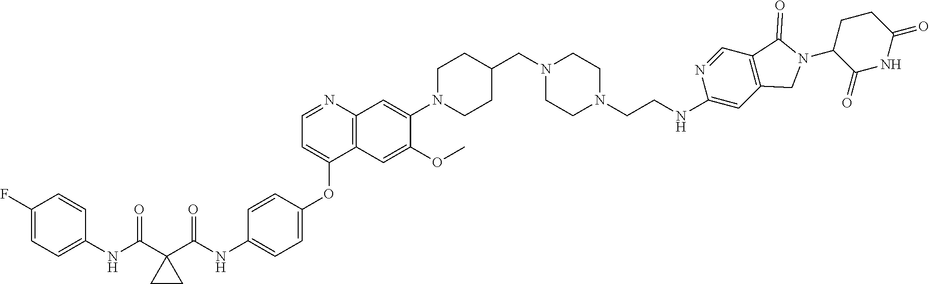 [SMILES: COc1cc2c(Oc3ccc(NC(=O)C4(C(=O)Nc5ccc(F)cc5)CC4)cc3)ccnc2cc1N1CCC(CN2CCN(CCNc3cc4c(cn3)C(=O)N(C3CCC(=O)NC3=O)C4)CC2)CC1]

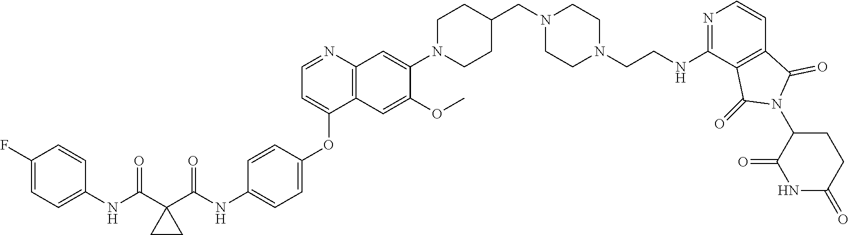 [SMILES: COc1cc2c(Oc3ccc(NC(=O)C4(C(=O)Nc5ccc(F)cc5)CC4)cc3)ccnc2cc1N1CCC(CN2CCN(CCNc3nccc4c3C(=O)N(C3CCC(=O)NC3=O)C4=O)CC2)CC1]

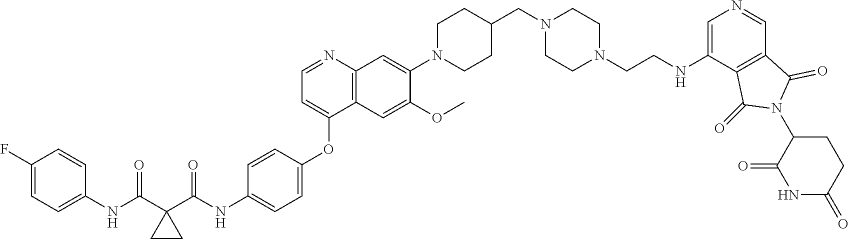 [SMILES: COc1cc2c(Oc3ccc(NC(=O)C4(C(=O)Nc5ccc(F)cc5)CC4)cc3)ccnc2cc1N1CCC(CN2CCN(CCNc3cncc4c3C(=O)N(C3CCC(=O)NC3=O)C4=O)CC2)CC1]

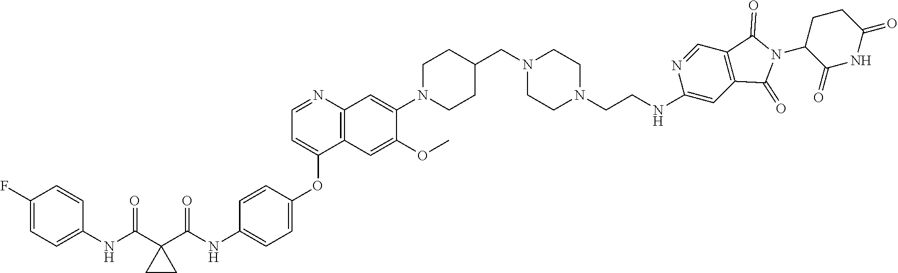 [SMILES: COc1cc2c(Oc3ccc(NC(=O)C4(C(=O)Nc5ccc(F)cc5)CC4)cc3)ccnc2cc1N1CCC(CN2CCN(CCNc3cc4c(cn3)C(=O)N(C3CCC(=O)NC3=O)C4=O)CC2)CC1]

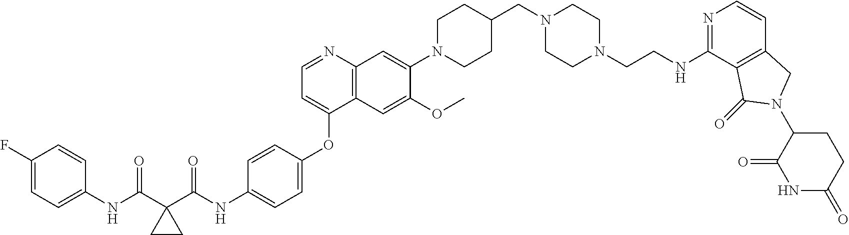 [SMILES: COc1cc2c(Oc3ccc(NC(=O)C4(C(=O)Nc5ccc(F)cc5)CC4)cc3)ccnc2cc1N1CCC(CN2CCN(CCNc3nccc4c3C(=O)N(C3CCC(=O)NC3=O)C4)CC2)CC1]

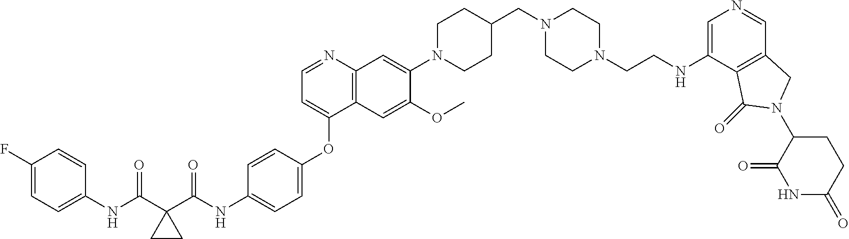 [SMILES: COc1cc2c(Oc3ccc(NC(=O)C4(C(=O)Nc5ccc(F)cc5)CC4)cc3)ccnc2cc1N1CCC(CN2CCN(CCNc3cncc4c3C(=O)N(C3CCC(=O)NC3=O)C4)CC2)CC1]

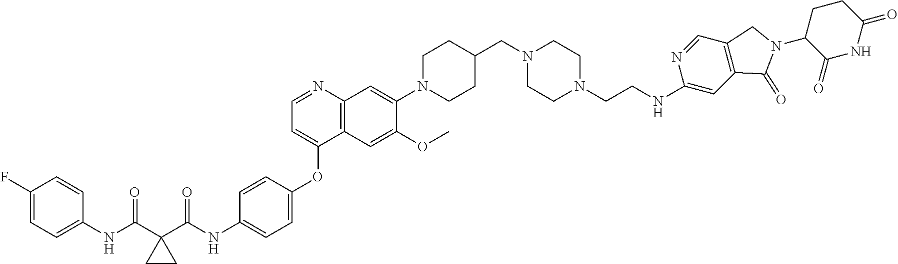 [SMILES: COc1cc2c(Oc3ccc(NC(=O)C4(C(=O)Nc5ccc(F)cc5)CC4)cc3)ccnc2cc1N1CCC(CN2CCN(CCNc3cc4c(cn3)CN(C3CCC(=O)NC3=O)C4=O)CC2)CC1]

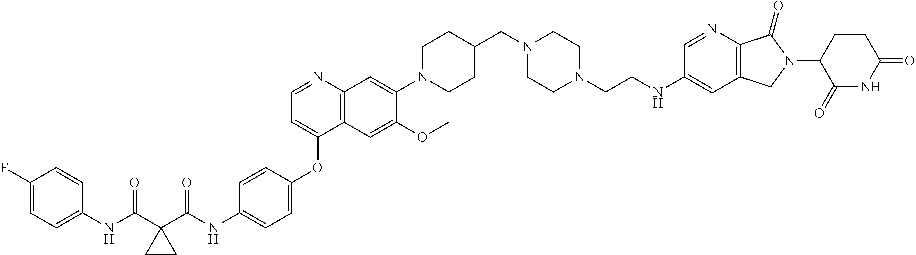 [SMILES: COc1cc2c(Oc3ccc(NC(=O)C4(C(=O)Nc5ccc(F)cc5)CC4)cc3)ccnc2cc1N1CCC(CN2CCN(CCNc3cnc4c(c3)CN(C3CCC(=O)NC3=O)C4=O)CC2)CC1]

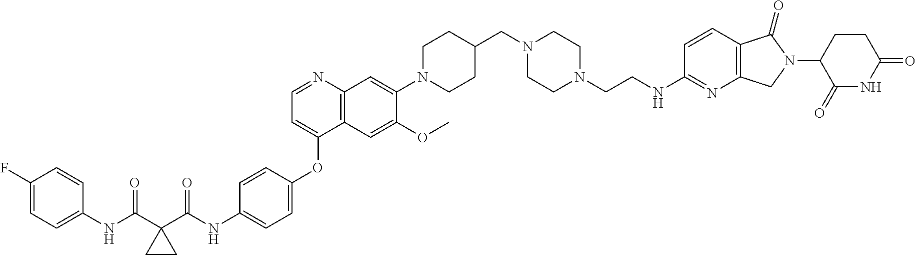 [SMILES: COc1cc2c(Oc3ccc(NC(=O)C4(C(=O)Nc5ccc(F)cc5)CC4)cc3)ccnc2cc1N1CCC(CN2CCN(CCNc3ccc4c(n3)CN(C3CCC(=O)NC3=O)C4=O)CC2)CC1]

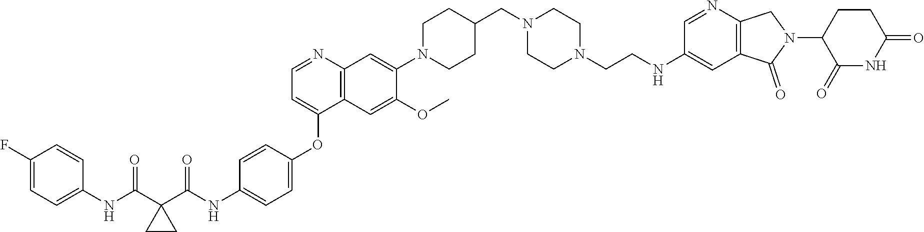 [SMILES: COc1cc2c(Oc3ccc(NC(=O)C4(C(=O)Nc5ccc(F)cc5)CC4)cc3)ccnc2cc1N1CCC(CN2CCN(CCNc3cnc4c(c3)C(=O)N(C3CCC(=O)NC3=O)C4)CC2)CC1]

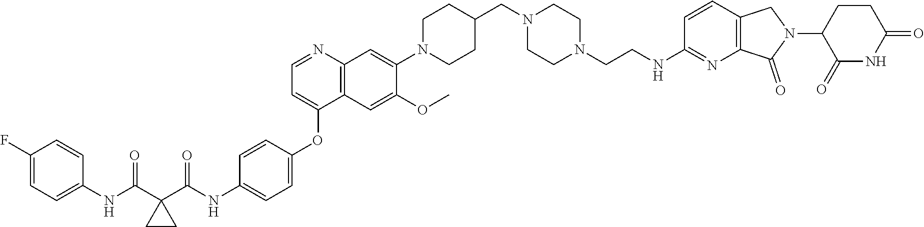 [SMILES: COc1cc2c(Oc3ccc(NC(=O)C4(C(=O)Nc5ccc(F)cc5)CC4)cc3)ccnc2cc1N1CCC(CN2CCN(CCNc3ccc4c(n3)C(=O)N(C3CCC(=O)NC3=O)C4)CC2)CC1]

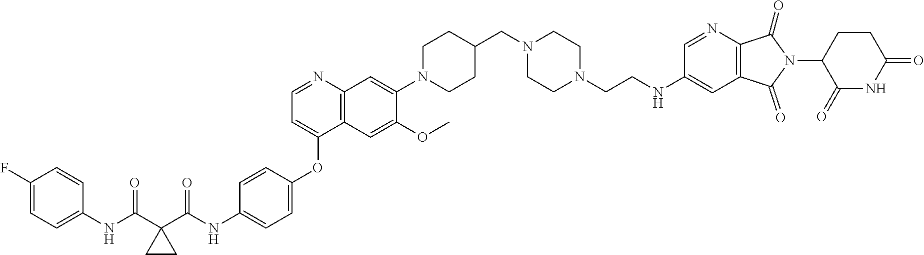 [SMILES: COc1cc2c(Oc3ccc(NC(=O)C4(C(=O)Nc5ccc(F)cc5)CC4)cc3)ccnc2cc1N1CCC(CN2CCN(CCNc3cnc4c(c3)C(=O)N(C3CCC(=O)NC3=O)C4=O)CC2)CC1]

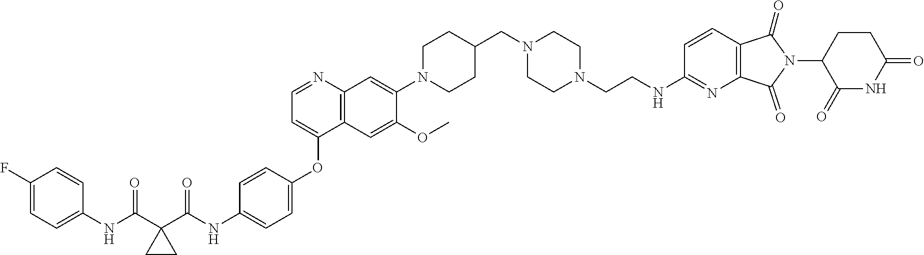 [SMILES: COc1cc2c(Oc3ccc(NC(=O)C4(C(=O)Nc5ccc(F)cc5)CC4)cc3)ccnc2cc1N1CCC(CN2CCN(CCNc3ccc4c(n3)C(=O)N(C3CCC(=O)NC3=O)C4=O)CC2)CC1]

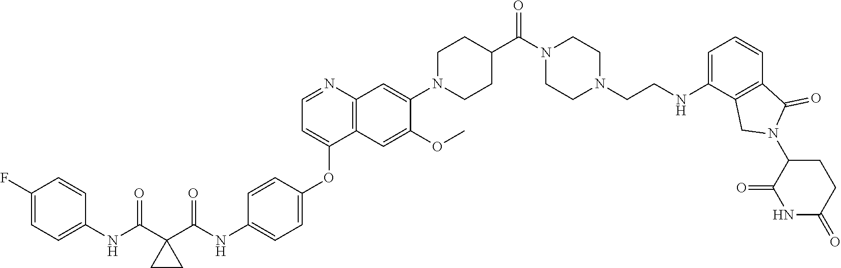 [SMILES: COc1cc2c(Oc3ccc(NC(=O)C4(C(=O)Nc5ccc(F)cc5)CC4)cc3)ccnc2cc1N1CCC(C(=O)N2CCN(CCNc3cccc4c3CN(C3CCC(=O)NC3=O)C4=O)CC2)CC1]

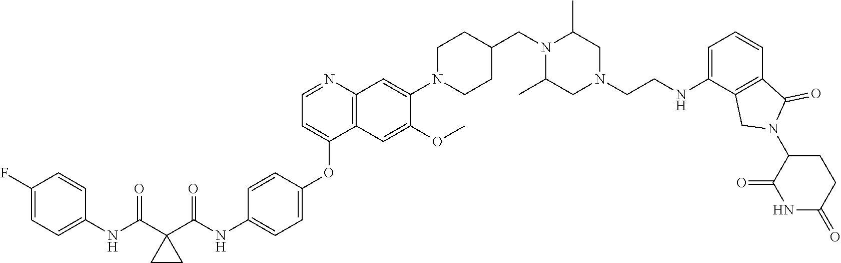 [SMILES: COc1cc2c(Oc3ccc(NC(=O)C4(C(=O)Nc5ccc(F)cc5)CC4)cc3)ccnc2cc1N1CCC(CN2C(C)CN(CCNc3cccc4c3CN(C3CCC(=O)NC3=O)C4=O)CC2C)CC1]

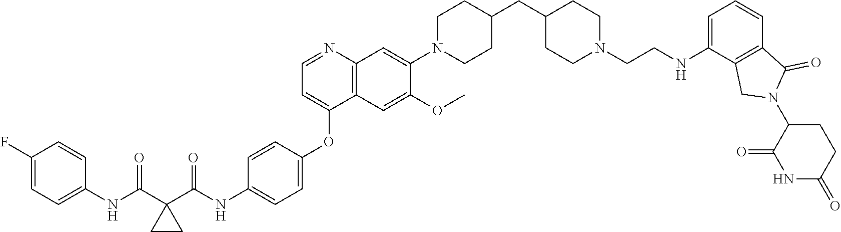 [SMILES: COc1cc2c(Oc3ccc(NC(=O)C4(C(=O)Nc5ccc(F)cc5)CC4)cc3)ccnc2cc1N1CCC(CC2CCN(CCNc3cccc4c3CN(C3CCC(=O)NC3=O)C4=O)CC2)CC1]

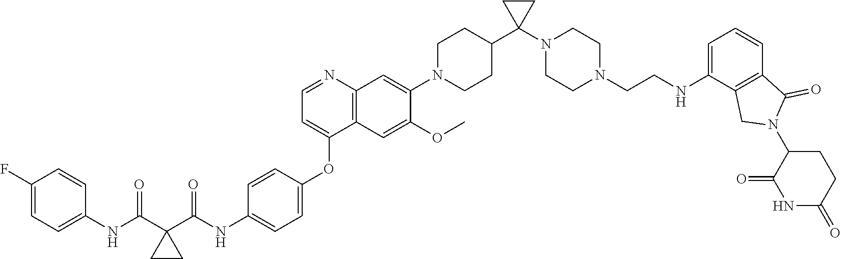 [SMILES: COc1cc2c(Oc3ccc(NC(=O)C4(C(=O)Nc5ccc(F)cc5)CC4)cc3)ccnc2cc1N1CCC(C2(N3CCN(CCNc4cccc5c4CN(C4CCC(=O)NC4=O)C5=O)CC3)CC2)CC1]

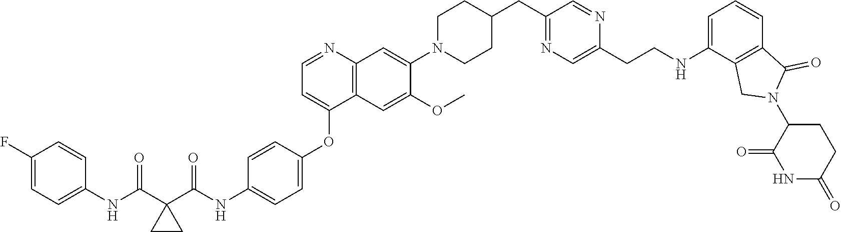 [SMILES: COc1cc2c(Oc3ccc(NC(=O)C4(C(=O)Nc5ccc(F)cc5)CC4)cc3)ccnc2cc1N1CCC(Cc2cnc(CCNc3cccc4c3CN(C3CCC(=O)NC3=O)C4=O)cn2)CC1]

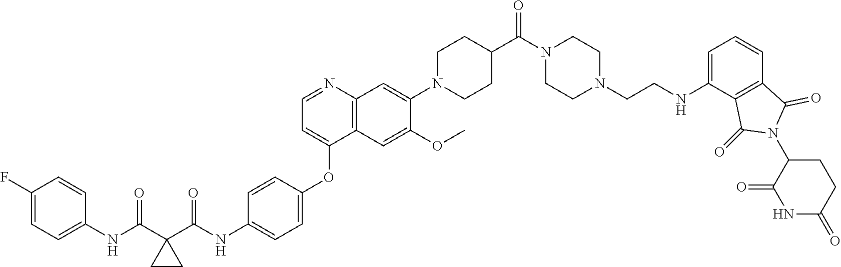 [SMILES: COc1cc2c(Oc3ccc(NC(=O)C4(C(=O)Nc5ccc(F)cc5)CC4)cc3)ccnc2cc1N1CCC(C(=O)N2CCN(CCNc3cccc4c3C(=O)N(C3CCC(=O)NC3=O)C4=O)CC2)CC1]

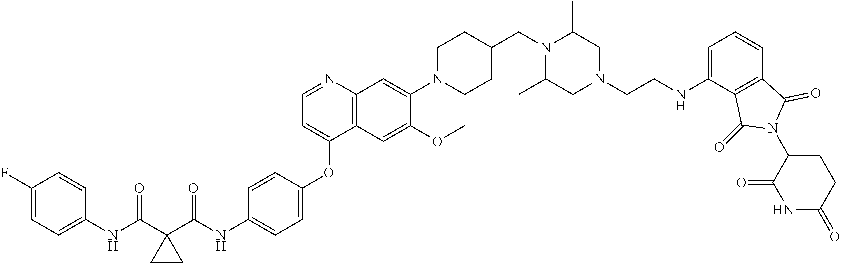 [SMILES: COc1cc2c(Oc3ccc(NC(=O)C4(C(=O)Nc5ccc(F)cc5)CC4)cc3)ccnc2cc1N1CCC(CN2C(C)CN(CCNc3cccc4c3C(=O)N(C3CCC(=O)NC3=O)C4=O)CC2C)CC1]

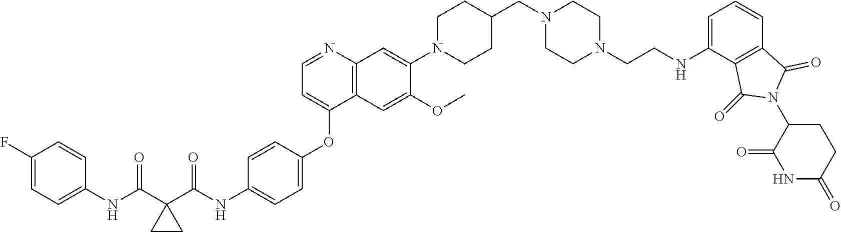 [SMILES: COc1cc2c(Oc3ccc(NC(=O)C4(C(=O)Nc5ccc(F)cc5)CC4)cc3)ccnc2cc1N1CCC(CN2CCN(CCNc3cccc4c3C(=O)N(C3CCC(=O)NC3=O)C4=O)CC2)CC1]

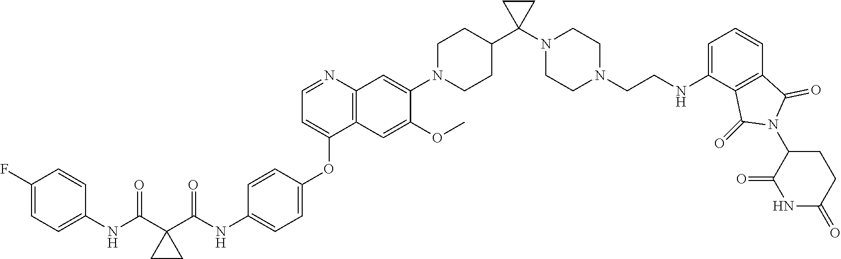 [SMILES: COc1cc2c(Oc3ccc(NC(=O)C4(C(=O)Nc5ccc(F)cc5)CC4)cc3)ccnc2cc1N1CCC(C2(N3CCN(CCNc4cccc5c4C(=O)N(C4CCC(=O)NC4=O)C5=O)CC3)CC2)CC1]

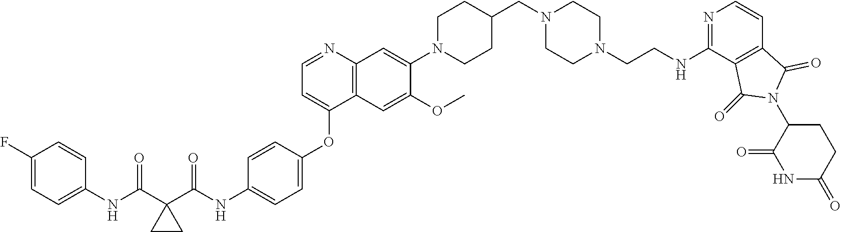 [SMILES: COc1cc2c(Oc3ccc(NC(=O)C4(C(=O)Nc5ccc(F)cc5)CC4)cc3)ccnc2cc1N1CCC(CN2CCN(CCNc3nccc4c3C(=O)N(C3CCC(=O)NC3=O)C4=O)CC2)CC1]

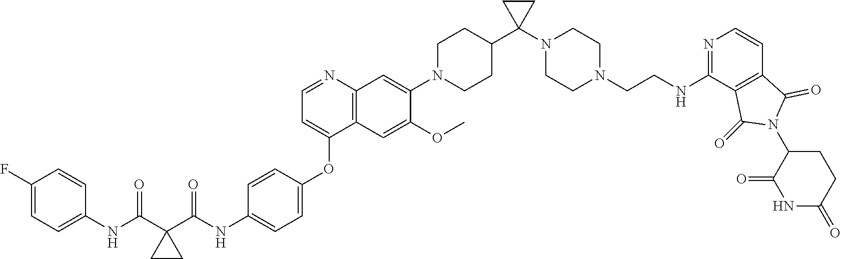 [SMILES: COc1cc2c(Oc3ccc(NC(=O)C4(C(=O)Nc5ccc(F)cc5)CC4)cc3)ccnc2cc1N1CCC(C2(N3CCN(CCNc4nccc5c4C(=O)N(C4CCC(=O)NC4=O)C5=O)CC3)CC2)CC1]

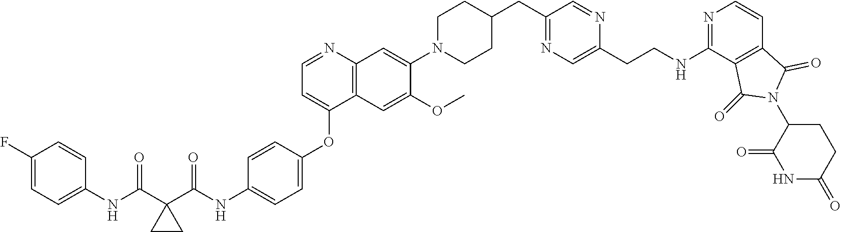 [SMILES: COc1cc2c(Oc3ccc(NC(=O)C4(C(=O)Nc5ccc(F)cc5)CC4)cc3)ccnc2cc1N1CCC(Cc2cnc(CCNc3nccc4c3C(=O)N(C3CCC(=O)NC3=O)C4=O)cn2)CC1]

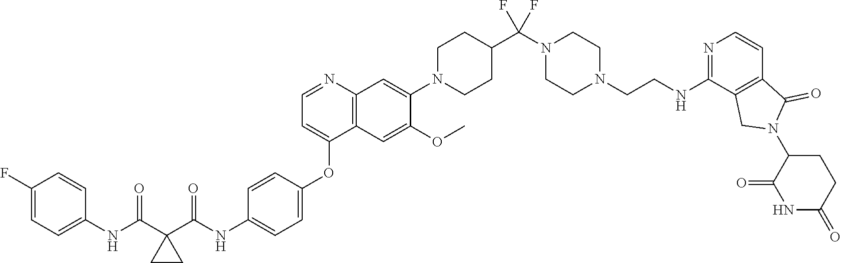 [SMILES: COc1cc2c(Oc3ccc(NC(=O)C4(C(=O)Nc5ccc(F)cc5)CC4)cc3)ccnc2cc1N1CCC(C(F)(F)N2CCN(CCNc3nccc4c3CN(C3CCC(=O)NC3=O)C4=O)CC2)CC1]

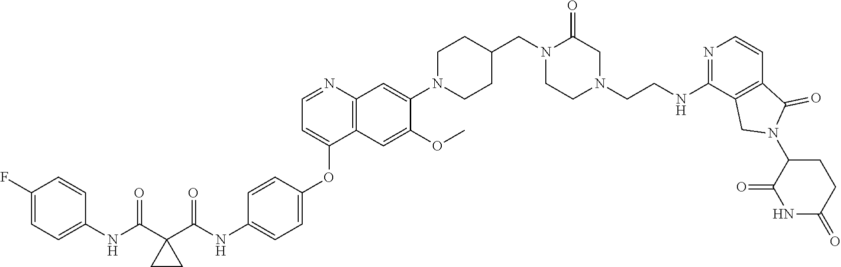 [SMILES: COc1cc2c(Oc3ccc(NC(=O)C4(C(=O)Nc5ccc(F)cc5)CC4)cc3)ccnc2cc1N1CCC(CN2CCN(CCNc3nccc4c3CN(C3CCC(=O)NC3=O)C4=O)CC2=O)CC1]

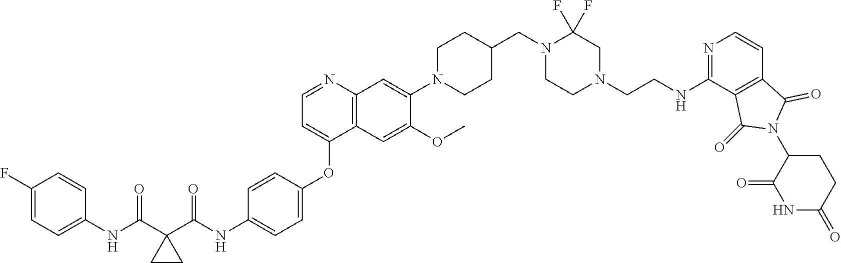 [SMILES: COc1cc2c(Oc3ccc(NC(=O)C4(C(=O)Nc5ccc(F)cc5)CC4)cc3)ccnc2cc1N1CCC(CN2CCN(CCNc3nccc4c3C(=O)N(C3CCC(=O)NC3=O)C4=O)CC2(F)F)CC1]

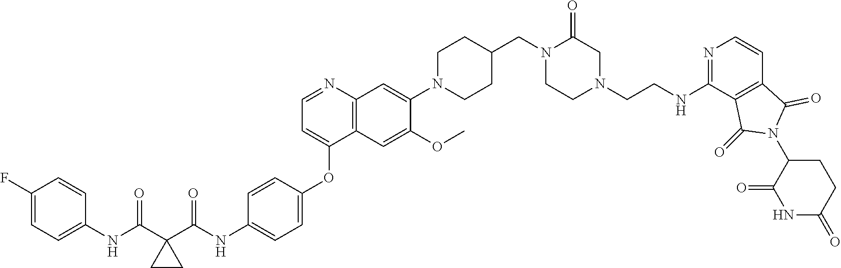 [SMILES: COc1cc2c(Oc3ccc(NC(=O)C4(C(=O)Nc5ccc(F)cc5)CC4)cc3)ccnc2cc1N1CCC(CN2CCN(CCNc3nccc4c3C(=O)N(C3CCC(=O)NC3=O)C4=O)CC2=O)CC1]

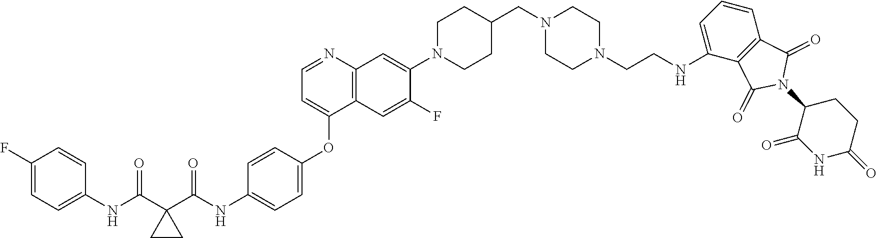 [SMILES: O=C1CC[C@H](N2C(=O)c3cccc(NCCN4CCN(CC5CCN(c6cc7nccc(Oc8ccc(NC(=O)C9(C(=O)Nc%10ccc(F)cc%10)CC9)cc8)c7cc6F)CC5)CC4)c3C2=O)C(=O)N1]

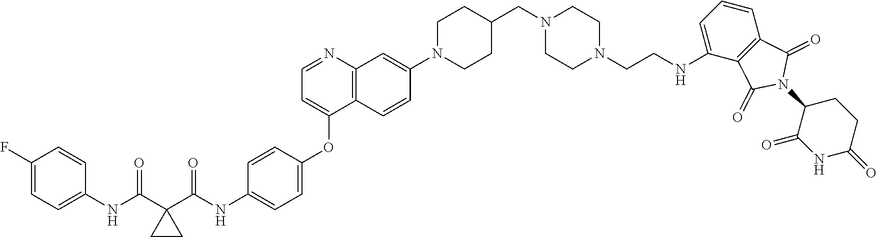 [SMILES: O=C1CC[C@H](N2C(=O)c3cccc(NCCN4CCN(CC5CCN(c6ccc7c(Oc8ccc(NC(=O)C9(C(=O)Nc%10ccc(F)cc%10)CC9)cc8)ccnc7c6)CC5)CC4)c3C2=O)C(=O)N1]

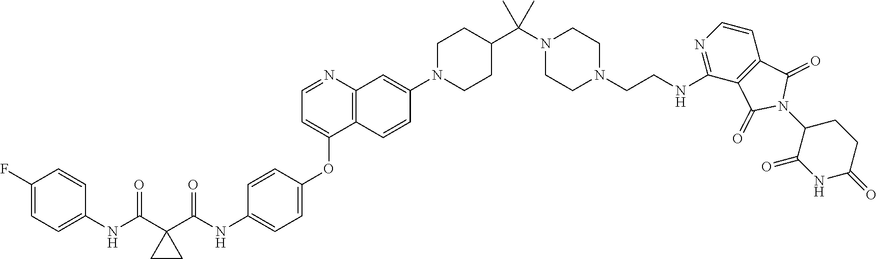 [SMILES: CC(C)(C1CCN(c2ccc3c(Oc4ccc(NC(=O)C5(C(=O)Nc6ccc(F)cc6)CC5)cc4)ccnc3c2)CC1)N1CCN(CCNc2nccc3c2C(=O)N(C2CCC(=O)NC2=O)C3=O)CC1]

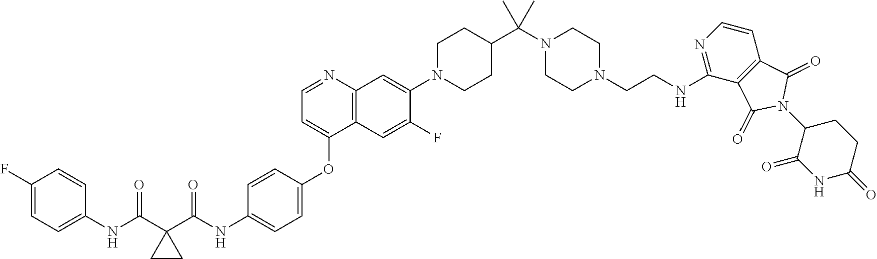 [SMILES: CC(C)(C1CCN(c2cc3nccc(Oc4ccc(NC(=O)C5(C(=O)Nc6ccc(F)cc6)CC5)cc4)c3cc2F)CC1)N1CCN(CCNc2nccc3c2C(=O)N(C2CCC(=O)NC2=O)C3=O)CC1]

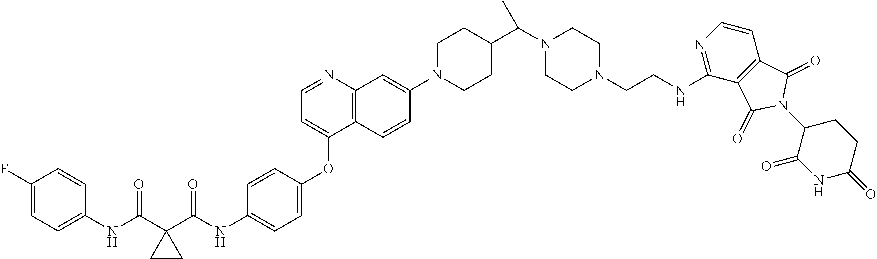 [SMILES: CC(C1CCN(c2ccc3c(Oc4ccc(NC(=O)C5(C(=O)Nc6ccc(F)cc6)CC5)cc4)ccnc3c2)CC1)N1CCN(CCNc2nccc3c2C(=O)N(C2CCC(=O)NC2=O)C3=O)CC1]

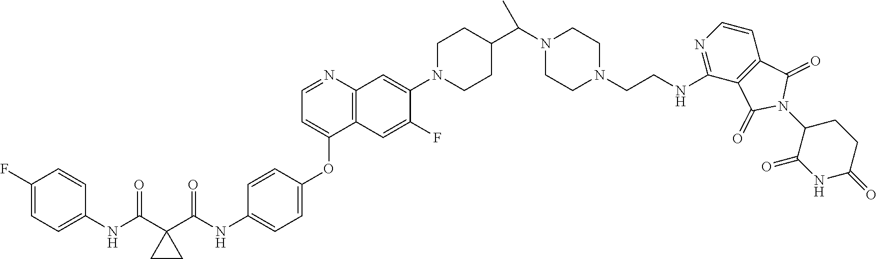 [SMILES: CC(C1CCN(c2cc3nccc(Oc4ccc(NC(=O)C5(C(=O)Nc6ccc(F)cc6)CC5)cc4)c3cc2F)CC1)N1CCN(CCNc2nccc3c2C(=O)N(C2CCC(=O)NC2=O)C3=O)CC1]

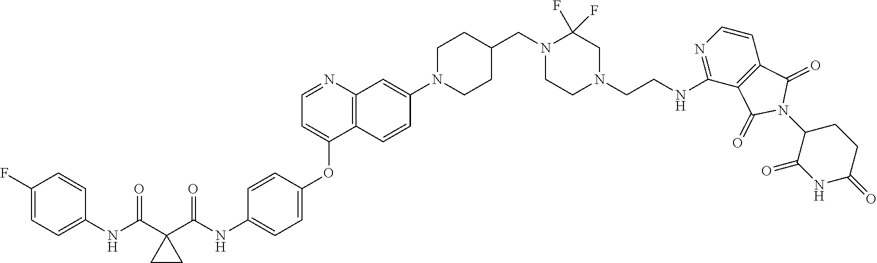 [SMILES: O=C1CCC(N2C(=O)c3ccnc(NCCN4CCN(CC5CCN(c6ccc7c(Oc8ccc(NC(=O)C9(C(=O)Nc%10ccc(F)cc%10)CC9)cc8)ccnc7c6)CC5)C(F)(F)C4)c3C2=O)C(=O)N1]

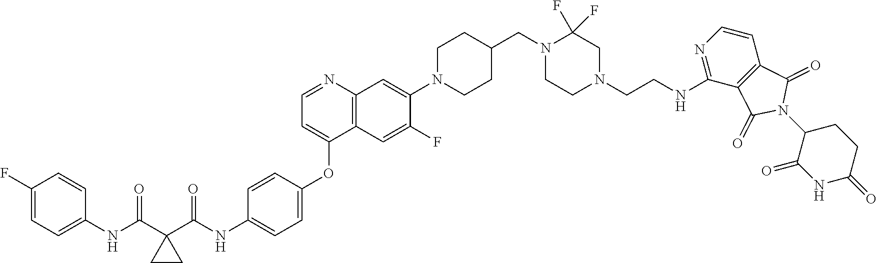 [SMILES: O=C1CCC(N2C(=O)c3ccnc(NCCN4CCN(CC5CCN(c6cc7nccc(Oc8ccc(NC(=O)C9(C(=O)Nc%10ccc(F)cc%10)CC9)cc8)c7cc6F)CC5)C(F)(F)C4)c3C2=O)C(=O)N1]

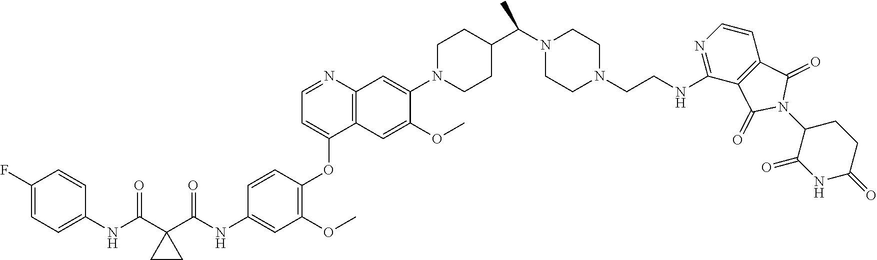 [SMILES: COc1cc(NC(=O)C2(C(=O)Nc3ccc(F)cc3)CC2)ccc1Oc1ccnc2cc(N3CCC([C@@H](C)N4CCN(CCNc5nccc6c5C(=O)N(C5CCC(=O)NC5=O)C6=O)CC4)CC3)c(OC)cc12]

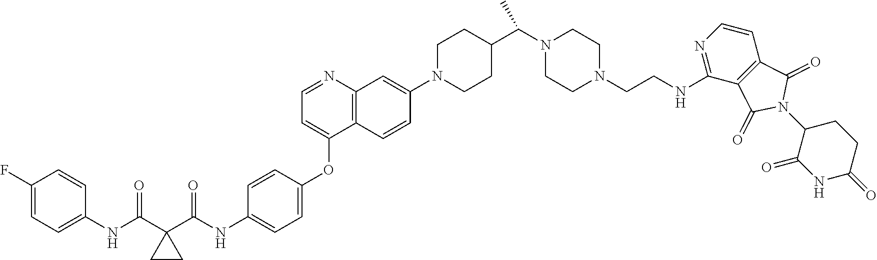 [SMILES: C[C@@H](C1CCN(c2ccc3c(Oc4ccc(NC(=O)C5(C(=O)Nc6ccc(F)cc6)CC5)cc4)ccnc3c2)CC1)N1CCN(CCNc2nccc3c2C(=O)N(C2CCC(=O)NC2=O)C3=O)CC1]

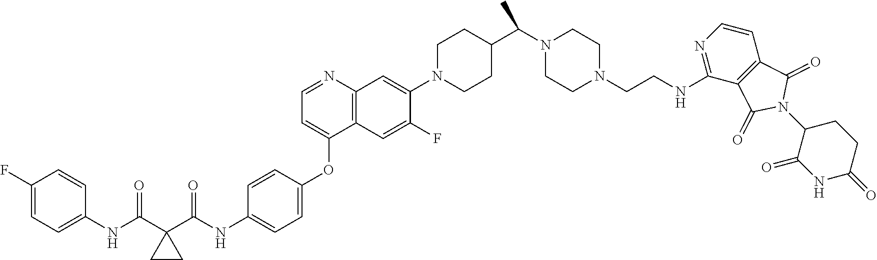 [SMILES: C[C@H](C1CCN(c2cc3nccc(Oc4ccc(NC(=O)C5(C(=O)Nc6ccc(F)cc6)CC5)cc4)c3cc2F)CC1)N1CCN(CCNc2nccc3c2C(=O)N(C2CCC(=O)NC2=O)C3=O)CC1]

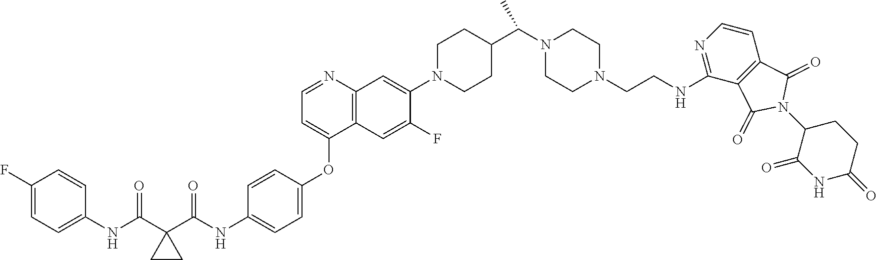 [SMILES: C[C@@H](C1CCN(c2cc3nccc(Oc4ccc(NC(=O)C5(C(=O)Nc6ccc(F)cc6)CC5)cc4)c3cc2F)CC1)N1CCN(CCNc2nccc3c2C(=O)N(C2CCC(=O)NC2=O)C3=O)CC1]

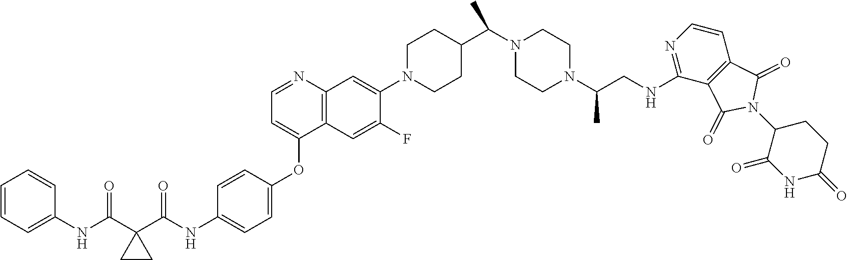 [SMILES: C[C@H](CNc1nccc2c1C(=O)N(C1CCC(=O)NC1=O)C2=O)N1CCN([C@H](C)C2CCN(c3cc4nccc(Oc5ccc(NC(=O)C6(C(=O)Nc7ccccc7)CC6)cc5)c4cc3F)CC2)CC1]

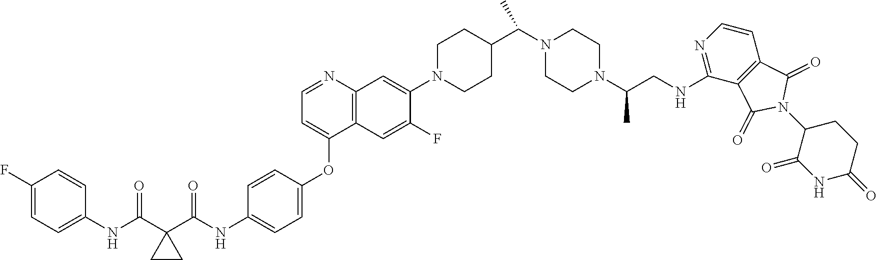 [SMILES: C[C@H](CNc1nccc2c1C(=O)N(C1CCC(=O)NC1=O)C2=O)N1CCN([C@@H](C)C2CCN(c3cc4nccc(Oc5ccc(NC(=O)C6(C(=O)Nc7ccc(F)cc7)CC6)cc5)c4cc3F)CC2)CC1]

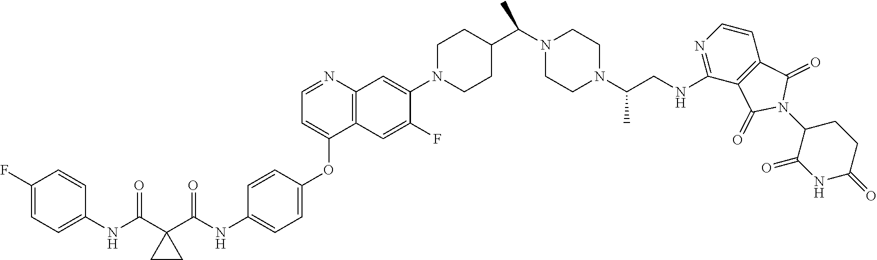 [SMILES: C[C@H](C1CCN(c2cc3nccc(Oc4ccc(NC(=O)C5(C(=O)Nc6ccc(F)cc6)CC5)cc4)c3cc2F)CC1)N1CCN([C@@H](C)CNc2nccc3c2C(=O)N(C2CCC(=O)NC2=O)C3=O)CC1]

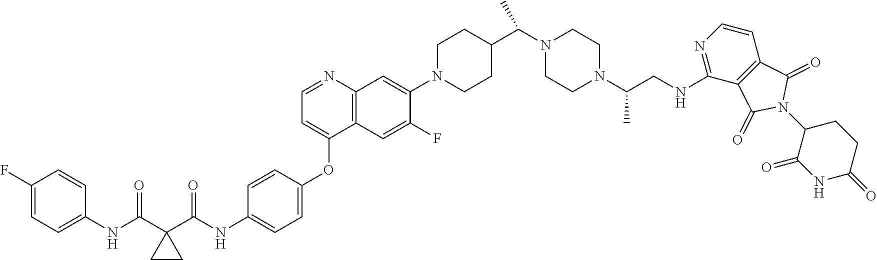 [SMILES: C[C@@H](CNc1nccc2c1C(=O)N(C1CCC(=O)NC1=O)C2=O)N1CCN([C@@H](C)C2CCN(c3cc4nccc(Oc5ccc(NC(=O)C6(C(=O)Nc7ccc(F)cc7)CC6)cc5)c4cc3F)CC2)CC1]

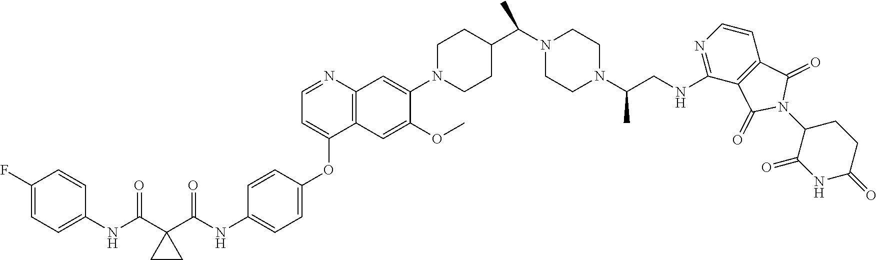 [SMILES: COc1cc2c(Oc3ccc(NC(=O)C4(C(=O)Nc5ccc(F)cc5)CC4)cc3)ccnc2cc1N1CCC([C@@H](C)N2CCN([C@H](C)CNc3nccc4c3C(=O)N(C3CCC(=O)NC3=O)C4=O)CC2)CC1]

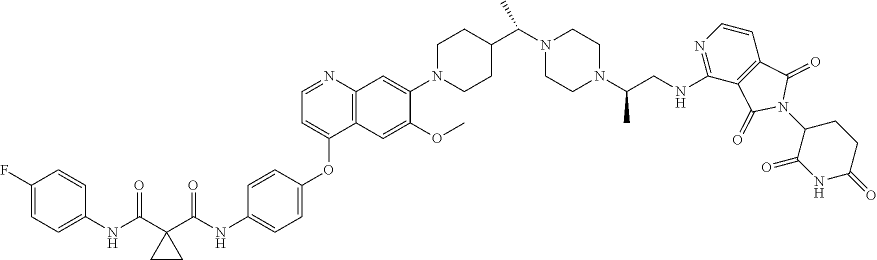 [SMILES: COc1cc2c(Oc3ccc(NC(=O)C4(C(=O)Nc5ccc(F)cc5)CC4)cc3)ccnc2cc1N1CCC([C@H](C)N2CCN([C@H](C)CNc3nccc4c3C(=O)N(C3CCC(=O)NC3=O)C4=O)CC2)CC1]

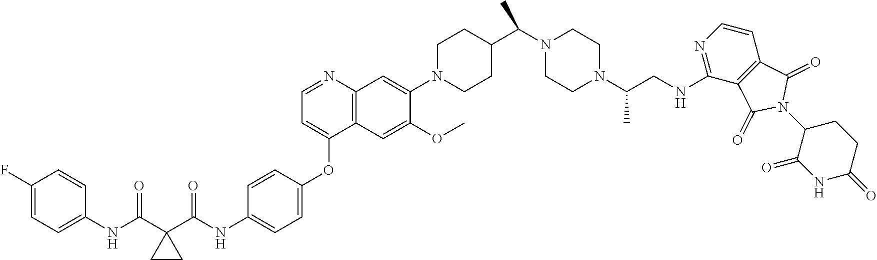 [SMILES: COc1cc2c(Oc3ccc(NC(=O)C4(C(=O)Nc5ccc(F)cc5)CC4)cc3)ccnc2cc1N1CCC([C@@H](C)N2CCN([C@@H](C)CNc3nccc4c3C(=O)N(C3CCC(=O)NC3=O)C4=O)CC2)CC1]

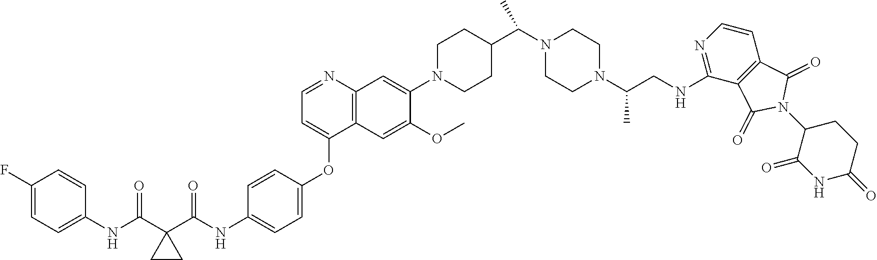 [SMILES: COc1cc2c(Oc3ccc(NC(=O)C4(C(=O)Nc5ccc(F)cc5)CC4)cc3)ccnc2cc1N1CCC([C@H](C)N2CCN([C@@H](C)CNc3nccc4c3C(=O)N(C3CCC(=O)NC3=O)C4=O)CC2)CC1]

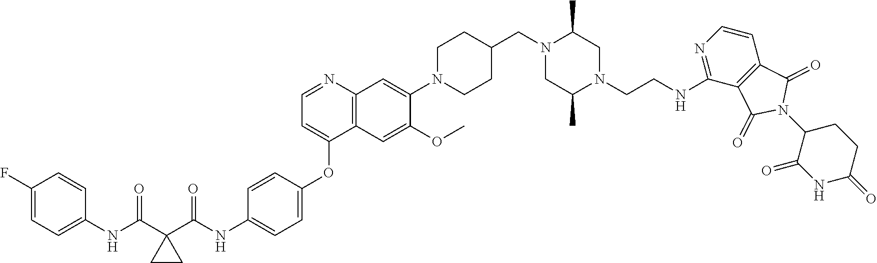 [SMILES: COc1cc2c(Oc3ccc(NC(=O)C4(C(=O)Nc5ccc(F)cc5)CC4)cc3)ccnc2cc1N1CCC(CN2C[C@H](C)N(CCNc3nccc4c3C(=O)N(C3CCC(=O)NC3=O)C4=O)C[C@@H]2C)CC1]

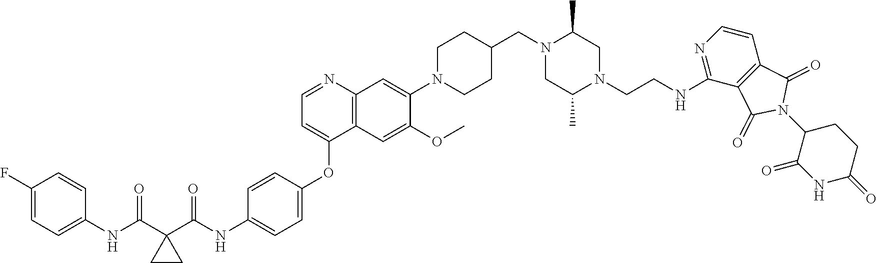 [SMILES: COc1cc2c(Oc3ccc(NC(=O)C4(C(=O)Nc5ccc(F)cc5)CC4)cc3)ccnc2cc1N1CCC(CN2C[C@@H](C)N(CCNc3nccc4c3C(=O)N(C3CCC(=O)NC3=O)C4=O)C[C@@H]2C)CC1]

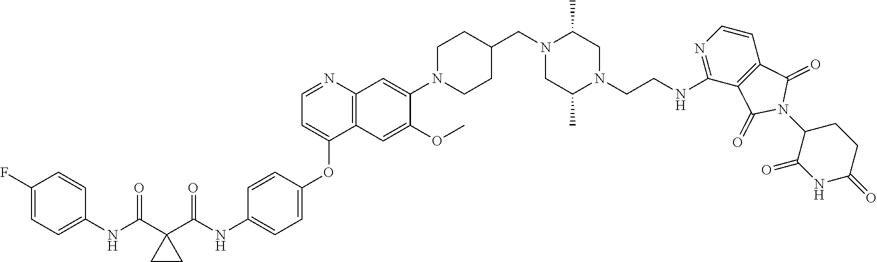 [SMILES: COc1cc2c(Oc3ccc(NC(=O)C4(C(=O)Nc5ccc(F)cc5)CC4)cc3)ccnc2cc1N1CCC(CN2C[C@@H](C)N(CCNc3nccc4c3C(=O)N(C3CCC(=O)NC3=O)C4=O)C[C@H]2C)CC1]

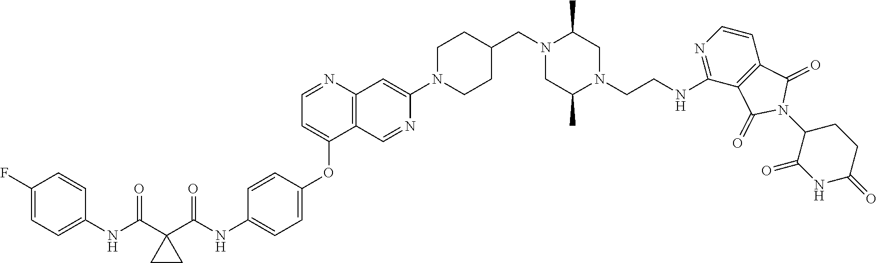 [SMILES: C[C@H]1CN(CC2CCN(c3cc4nccc(Oc5ccc(NC(=O)C6(C(=O)Nc7ccc(F)cc7)CC6)cc5)c4cn3)CC2)[C@@H](C)CN1CCNc1nccc2c1C(=O)N(C1CCC(=O)NC1=O)C2=O]

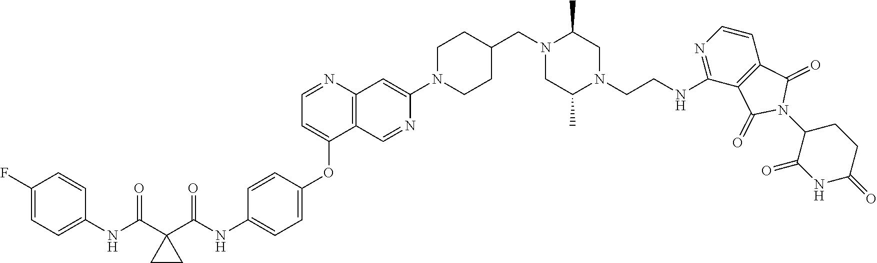 [SMILES: C[C@@H]1CN(CC2CCN(c3cc4nccc(Oc5ccc(NC(=O)C6(C(=O)Nc7ccc(F)cc7)CC6)cc5)c4cn3)CC2)[C@@H](C)CN1CCNc1nccc2c1C(=O)N(C1CCC(=O)NC1=O)C2=O]

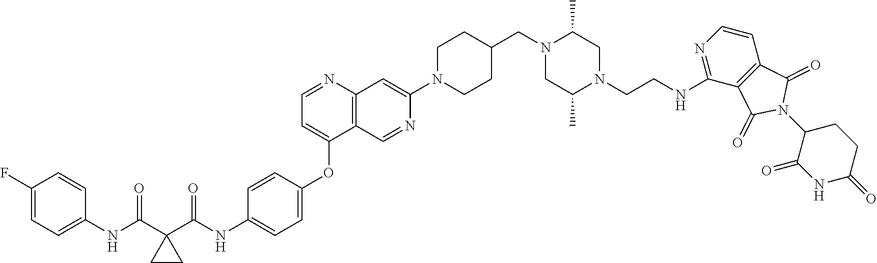 [SMILES: C[C@@H]1CN(CC2CCN(c3cc4nccc(Oc5ccc(NC(=O)C6(C(=O)Nc7ccc(F)cc7)CC6)cc5)c4cn3)CC2)[C@H](C)CN1CCNc1nccc2c1C(=O)N(C1CCC(=O)NC1=O)C2=O]

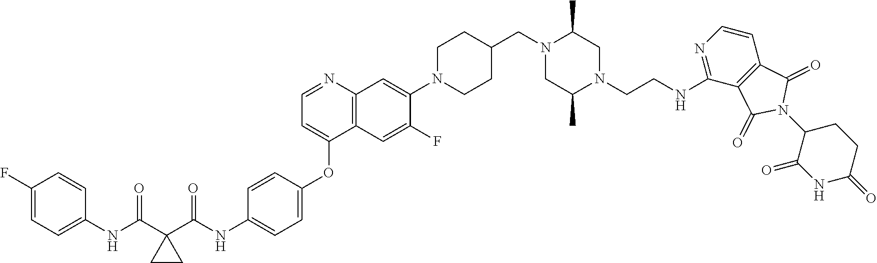 [SMILES: C[C@H]1CN(CC2CCN(c3cc4nccc(Oc5ccc(NC(=O)C6(C(=O)Nc7ccc(F)cc7)CC6)cc5)c4cc3F)CC2)[C@@H](C)CN1CCNc1nccc2c1C(=O)N(C1CCC(=O)NC1=O)C2=O]

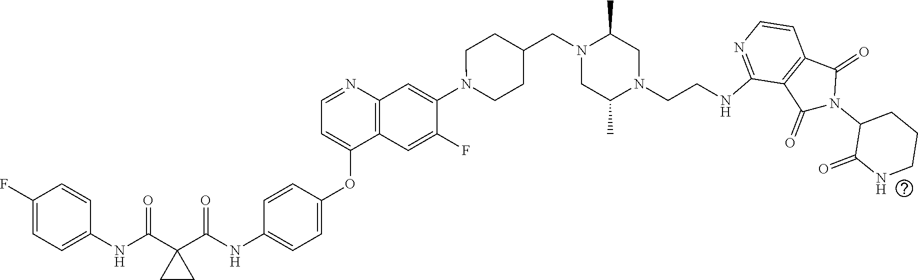 [SMILES: C[C@@H]1CN(CC2CCN(c3cc4nccc(Oc5ccc(NC(=O)C6(C(=O)Nc7ccc(F)cc7)CC6)cc5)c4cc3F)CC2)[C@@H](C)CN1CCNc1nccc2c1C(=O)N(C1CCCNC1=O)C2=O]

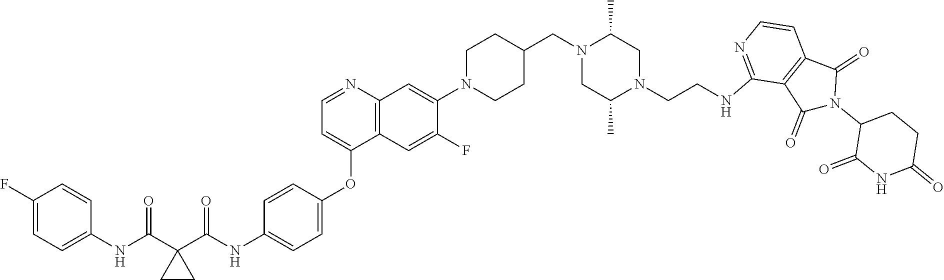 [SMILES: C[C@@H]1CN(CC2CCN(c3cc4nccc(Oc5ccc(NC(=O)C6(C(=O)Nc7ccc(F)cc7)CC6)cc5)c4cc3F)CC2)[C@H](C)CN1CCNc1nccc2c1C(=O)N(C1CCC(=O)NC1=O)C2=O]

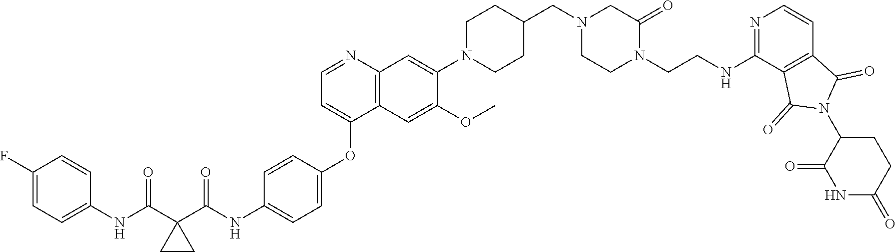 [SMILES: COc1cc2c(Oc3ccc(NC(=O)C4(C(=O)Nc5ccc(F)cc5)CC4)cc3)ccnc2cc1N1CCC(CN2CCN(CCNc3nccc4c3C(=O)N(C3CCC(=O)NC3=O)C4=O)C(=O)C2)CC1]

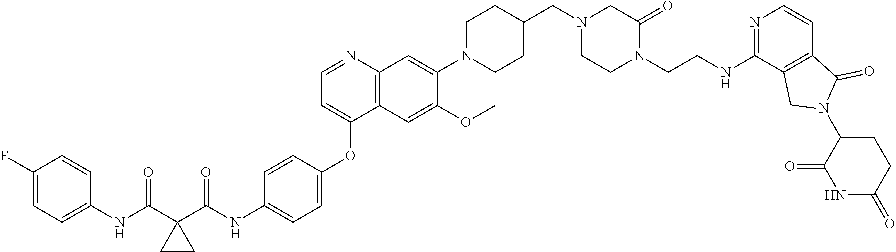 [SMILES: COc1cc2c(Oc3ccc(NC(=O)C4(C(=O)Nc5ccc(F)cc5)CC4)cc3)ccnc2cc1N1CCC(CN2CCN(CCNc3nccc4c3CN(C3CCC(=O)NC3=O)C4=O)C(=O)C2)CC1]

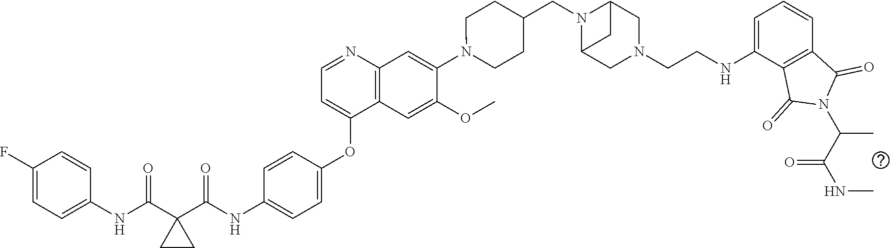 [SMILES: CNC(=O)C(C)N1C(=O)c2cccc(NCCN3CC4CC(C3)N4CC3CCN(c4cc5nccc(Oc6ccc(NC(=O)C7(C(=O)Nc8ccc(F)cc8)CC7)cc6)c5cc4OC)CC3)c2C1=O]

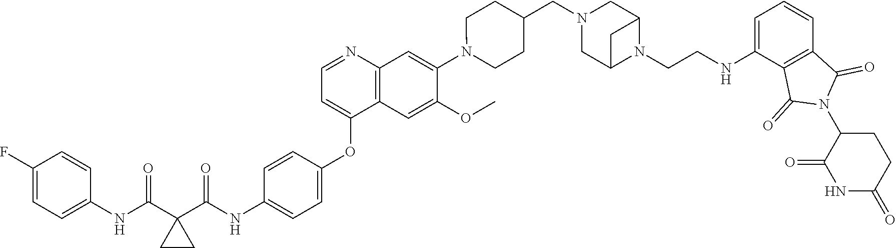 [SMILES: COc1cc2c(Oc3ccc(NC(=O)C4(C(=O)Nc5ccc(F)cc5)CC4)cc3)ccnc2cc1N1CCC(CN2CC3CC(C2)N3CCNc2cccc3c2C(=O)N(C2CCC(=O)NC2=O)C3=O)CC1]

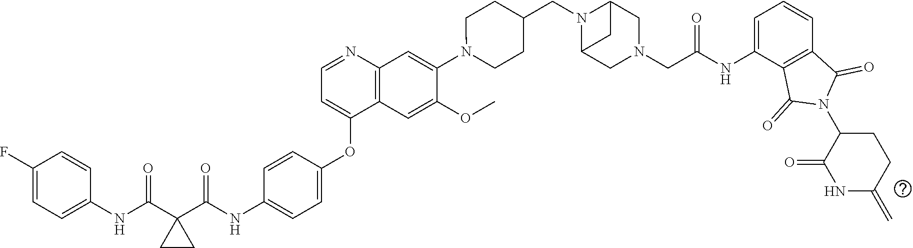 [SMILES: C=C1CCC(N2C(=O)c3cccc(NC(=O)CN4CC5CC(C4)N5CC4CCN(c5cc6nccc(Oc7ccc(NC(=O)C8(C(=O)Nc9ccc(F)cc9)CC8)cc7)c6cc5OC)CC4)c3C2=O)C(=O)N1]

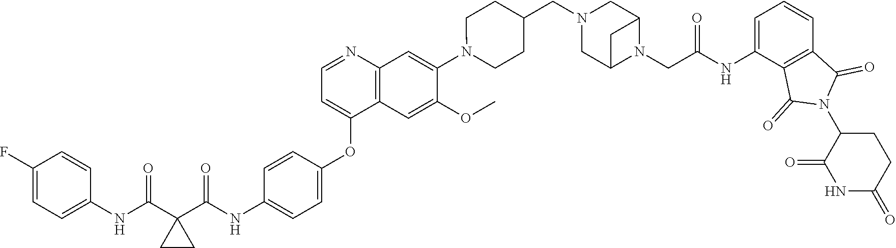 [SMILES: COc1cc2c(Oc3ccc(NC(=O)C4(C(=O)Nc5ccc(F)cc5)CC4)cc3)ccnc2cc1N1CCC(CN2CC3CC(C2)N3CC(=O)Nc2cccc3c2C(=O)N(C2CCC(=O)NC2=O)C3=O)CC1]

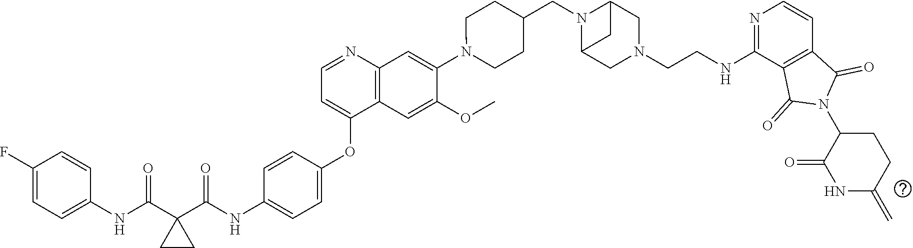 [SMILES: C=C1CCC(N2C(=O)c3ccnc(NCCN4CC5CC(C4)N5CC4CCN(c5cc6nccc(Oc7ccc(NC(=O)C8(C(=O)Nc9ccc(F)cc9)CC8)cc7)c6cc5OC)CC4)c3C2=O)C(=O)N1]

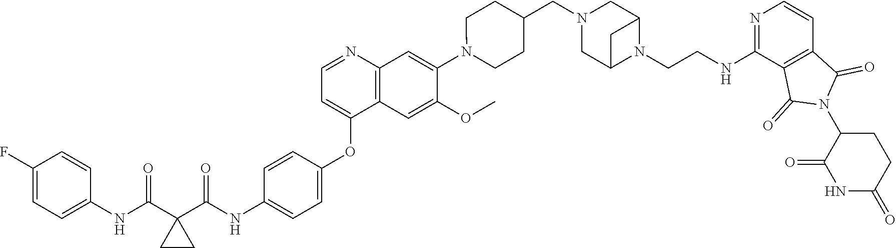 [SMILES: COc1cc2c(Oc3ccc(NC(=O)C4(C(=O)Nc5ccc(F)cc5)CC4)cc3)ccnc2cc1N1CCC(CN2CC3CC(C2)N3CCNc2nccc3c2C(=O)N(C2CCC(=O)NC2=O)C3=O)CC1]

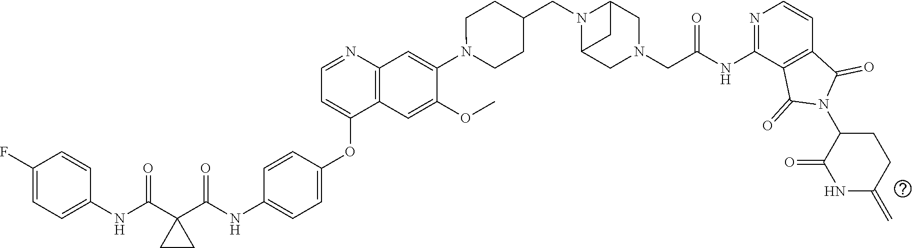 [SMILES: C=C1CCC(N2C(=O)c3ccnc(NC(=O)CN4CC5CC(C4)N5CC4CCN(c5cc6nccc(Oc7ccc(NC(=O)C8(C(=O)Nc9ccc(F)cc9)CC8)cc7)c6cc5OC)CC4)c3C2=O)C(=O)N1]

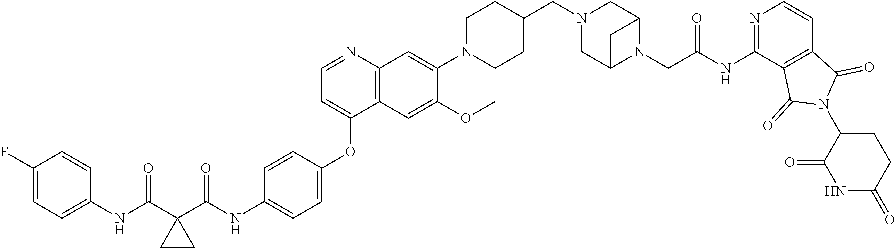 [SMILES: COc1cc2c(Oc3ccc(NC(=O)C4(C(=O)Nc5ccc(F)cc5)CC4)cc3)ccnc2cc1N1CCC(CN2CC3CC(C2)N3CC(=O)Nc2nccc3c2C(=O)N(C2CCC(=O)NC2=O)C3=O)CC1]

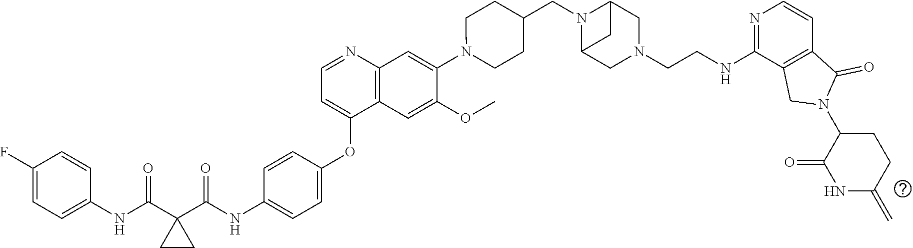 [SMILES: C=C1CCC(N2Cc3c(ccnc3NCCN3CC4CC(C3)N4CC3CCN(c4cc5nccc(Oc6ccc(NC(=O)C7(C(=O)Nc8ccc(F)cc8)CC7)cc6)c5cc4OC)CC3)C2=O)C(=O)N1]

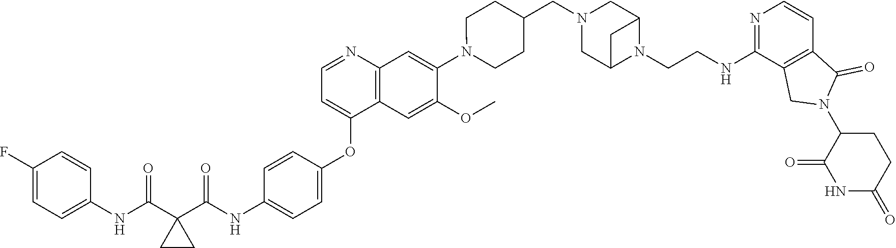 [SMILES: COc1cc2c(Oc3ccc(NC(=O)C4(C(=O)Nc5ccc(F)cc5)CC4)cc3)ccnc2cc1N1CCC(CN2CC3CC(C2)N3CCNc2nccc3c2CN(C2CCC(=O)NC2=O)C3=O)CC1]

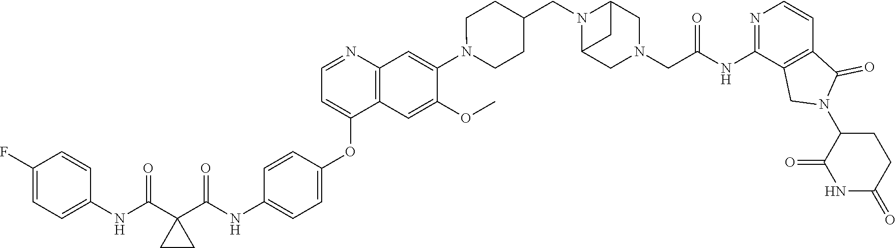 [SMILES: COc1cc2c(Oc3ccc(NC(=O)C4(C(=O)Nc5ccc(F)cc5)CC4)cc3)ccnc2cc1N1CCC(CN2C3CC2CN(CC(=O)Nc2nccc4c2CN(C2CCC(=O)NC2=O)C4=O)C3)CC1]

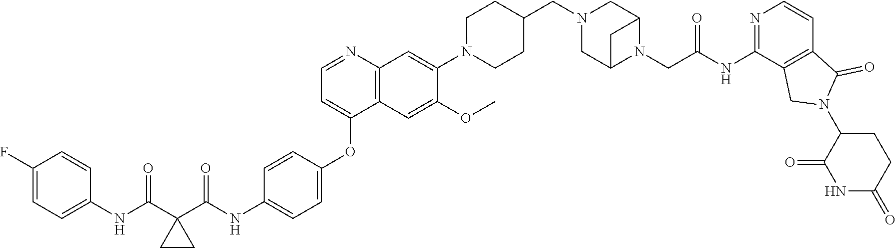 [SMILES: COc1cc2c(Oc3ccc(NC(=O)C4(C(=O)Nc5ccc(F)cc5)CC4)cc3)ccnc2cc1N1CCC(CN2CC3CC(C2)N3CC(=O)Nc2nccc3c2CN(C2CCC(=O)NC2=O)C3=O)CC1]

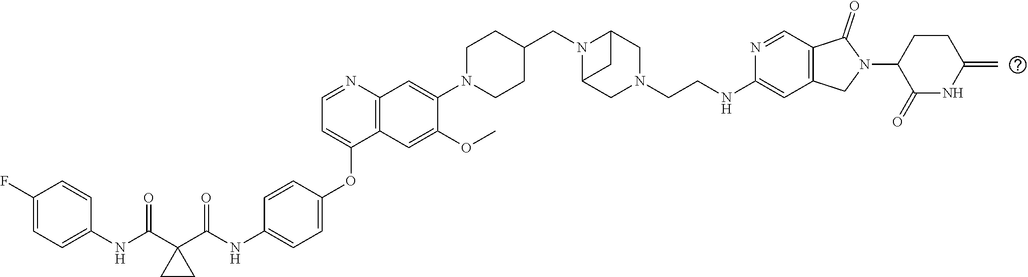 [SMILES: C=C1CCC(N2Cc3cc(NCCN4CC5CC(C4)N5CC4CCN(c5cc6nccc(Oc7ccc(NC(=O)C8(C(=O)Nc9ccc(F)cc9)CC8)cc7)c6cc5OC)CC4)ncc3C2=O)C(=O)N1]

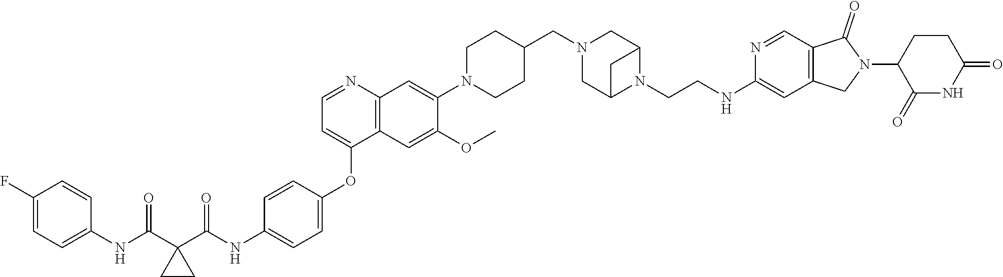 [SMILES: COc1cc2c(Oc3ccc(NC(=O)C4(C(=O)Nc5ccc(F)cc5)CC4)cc3)ccnc2cc1N1CCC(CN2CC3CC(C2)N3CCNc2cc3c(cn2)C(=O)N(C2CCC(=O)NC2=O)C3)CC1]

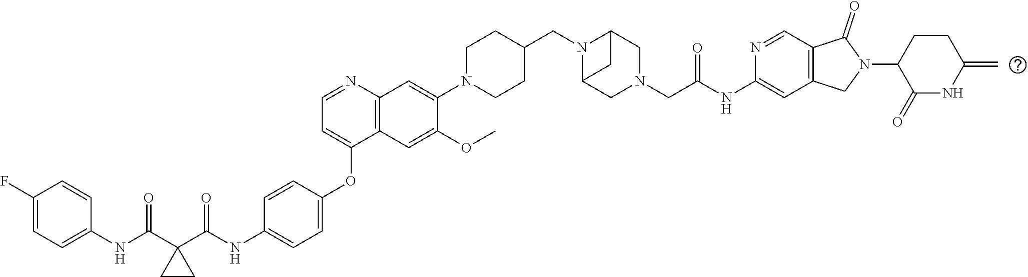 [SMILES: C=C1CCC(N2Cc3cc(NC(=O)CN4CC5CC(C4)N5CC4CCN(c5cc6nccc(Oc7ccc(NC(=O)C8(C(=O)Nc9ccc(F)cc9)CC8)cc7)c6cc5OC)CC4)ncc3C2=O)C(=O)N1]

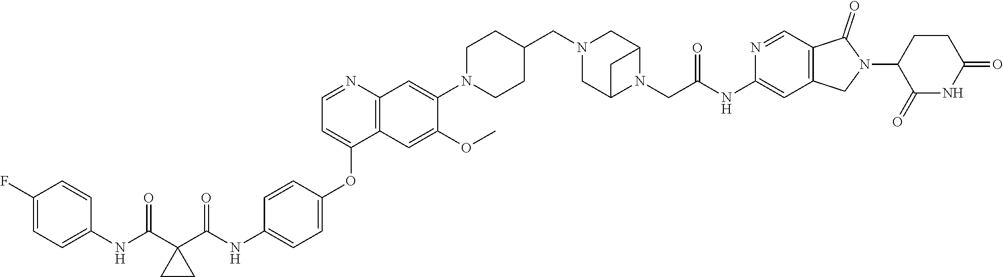 [SMILES: COc1cc2c(Oc3ccc(NC(=O)C4(C(=O)Nc5ccc(F)cc5)CC4)cc3)ccnc2cc1N1CCC(CN2CC3CC(C2)N3CC(=O)Nc2cc3c(cn2)C(=O)N(C2CCC(=O)NC2=O)C3)CC1]

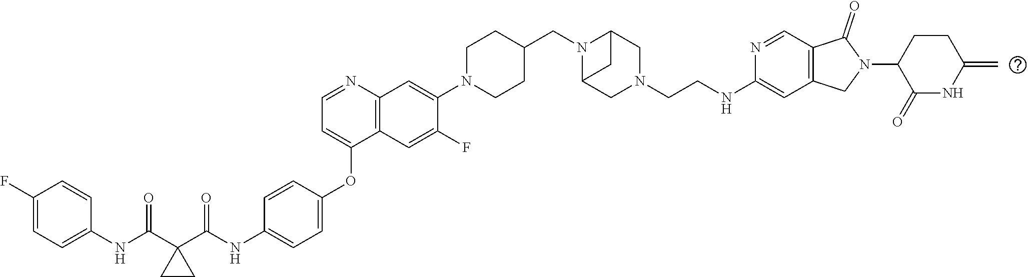 [SMILES: C=C1CCC(N2Cc3cc(NCCN4CC5CC(C4)N5CC4CCN(c5cc6nccc(Oc7ccc(NC(=O)C8(C(=O)Nc9ccc(F)cc9)CC8)cc7)c6cc5F)CC4)ncc3C2=O)C(=O)N1]

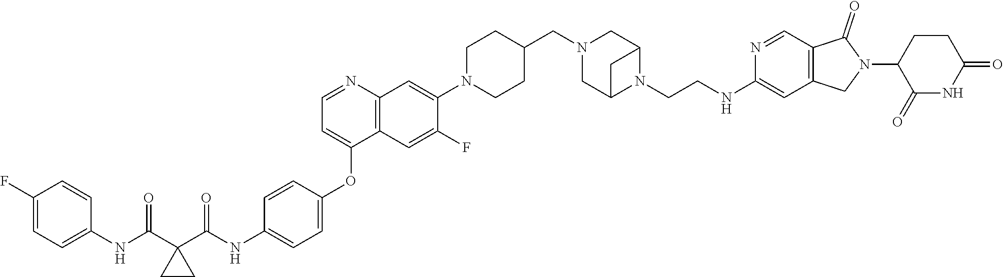 [SMILES: O=C1CCC(N2Cc3cc(NCCN4C5CC4CN(CC4CCN(c6cc7nccc(Oc8ccc(NC(=O)C9(C(=O)Nc%10ccc(F)cc%10)CC9)cc8)c7cc6F)CC4)C5)ncc3C2=O)C(=O)N1]

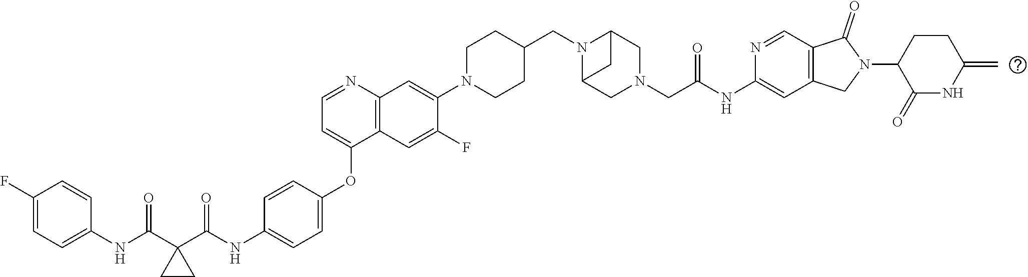 [SMILES: C=C1CCC(N2Cc3cc(NC(=O)CN4CC5CC(C4)N5CC4CCN(c5cc6nccc(Oc7ccc(NC(=O)C8(C(=O)Nc9ccc(F)cc9)CC8)cc7)c6cc5F)CC4)ncc3C2=O)C(=O)N1]

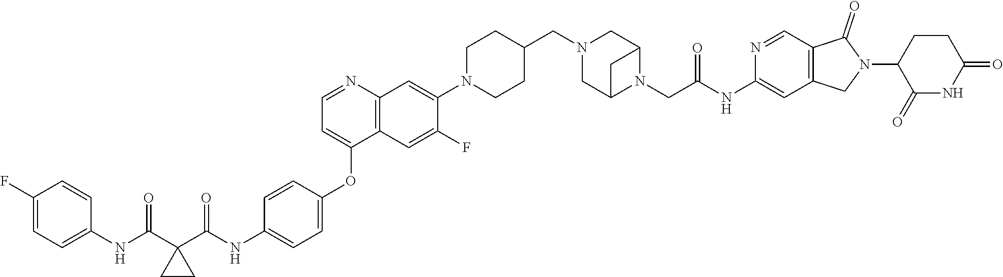 [SMILES: O=C1CCC(N2Cc3cc(NC(=O)CN4C5CC4CN(CC4CCN(c6cc7nccc(Oc8ccc(NC(=O)C9(C(=O)Nc%10ccc(F)cc%10)CC9)cc8)c7cc6F)CC4)C5)ncc3C2=O)C(=O)N1]

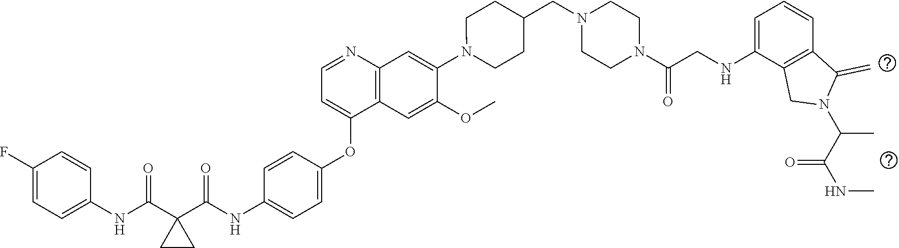 [SMILES: C=C1c2cccc(NCC(=O)N3CCN(CC4CCN(c5cc6nccc(Oc7ccc(NC(=O)C8(C(=O)Nc9ccc(F)cc9)CC8)cc7)c6cc5OC)CC4)CC3)c2CN1C(C)C(=O)NC]

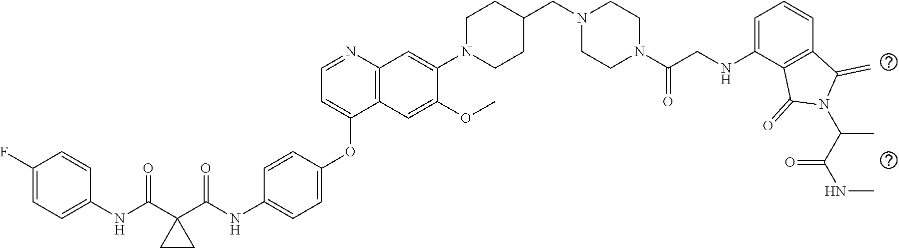 [SMILES: C=C1c2cccc(NCC(=O)N3CCN(CC4CCN(c5cc6nccc(Oc7ccc(NC(=O)C8(C(=O)Nc9ccc(F)cc9)CC8)cc7)c6cc5OC)CC4)CC3)c2C(=O)N1C(C)C(=O)NC]

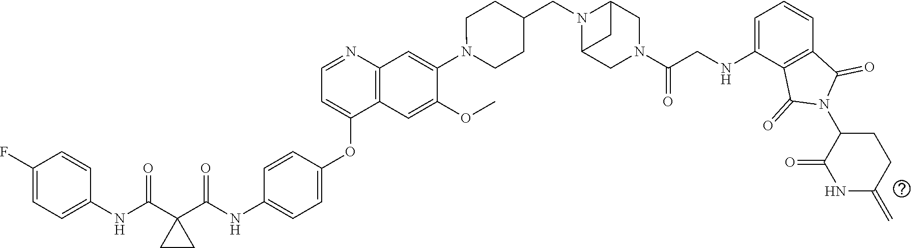 [SMILES: C=C1CCC(N2C(=O)c3cccc(NCC(=O)N4CC5CC(C4)N5CC4CCN(c5cc6nccc(Oc7ccc(NC(=O)C8(C(=O)Nc9ccc(F)cc9)CC8)cc7)c6cc5OC)CC4)c3C2=O)C(=O)N1]

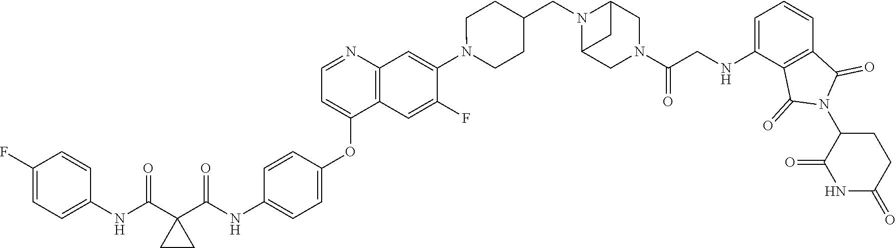 [SMILES: O=C1CCC(N2C(=O)c3cccc(NCC(=O)N4CC5CC(C4)N5CC4CCN(c5cc6nccc(Oc7ccc(NC(=O)C8(C(=O)Nc9ccc(F)cc9)CC8)cc7)c6cc5F)CC4)c3C2=O)C(=O)N1]

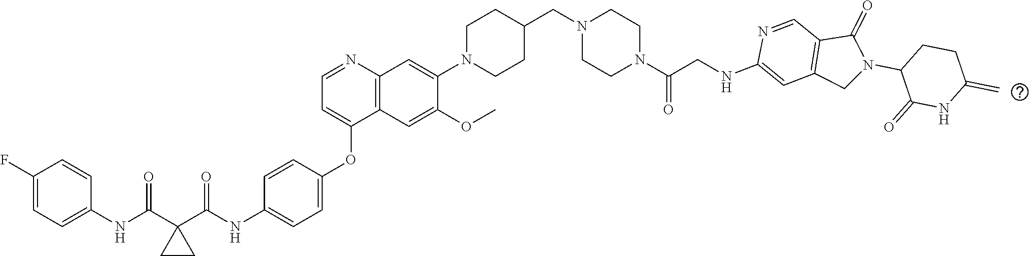 [SMILES: C=C1CCC(N2Cc3cc(NCC(=O)N4CCN(CC5CCN(c6cc7nccc(Oc8ccc(NC(=O)C9(C(=O)Nc%10ccc(F)cc%10)CC9)cc8)c7cc6OC)CC5)CC4)ncc3C2=O)C(=O)N1]

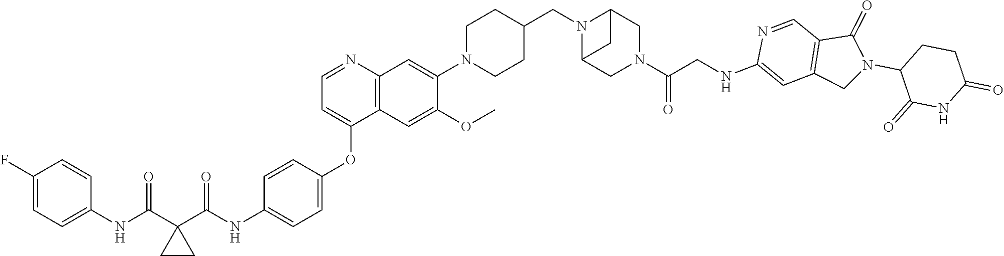 [SMILES: COc1cc2c(Oc3ccc(NC(=O)C4(C(=O)Nc5ccc(F)cc5)CC4)cc3)ccnc2cc1N1CCC(CN2C3CC2CN(C(=O)CNc2cc4c(cn2)C(=O)N(C2CCC(=O)NC2=O)C4)C3)CC1]

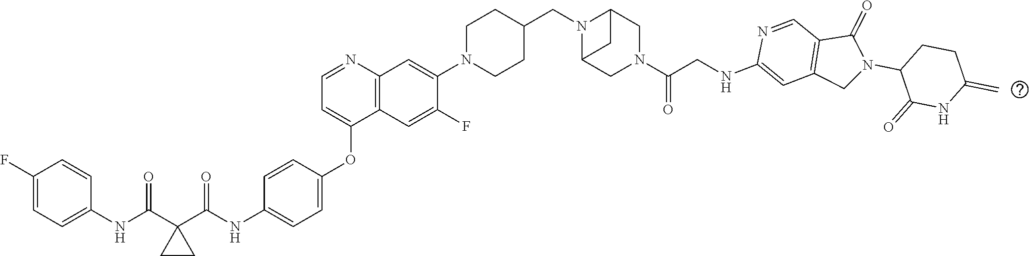 [SMILES: C=C1CCC(N2Cc3cc(NCC(=O)N4CC5CC(C4)N5CC4CCN(c5cc6nccc(Oc7ccc(NC(=O)C8(C(=O)Nc9ccc(F)cc9)CC8)cc7)c6cc5F)CC4)ncc3C2=O)C(=O)N1]

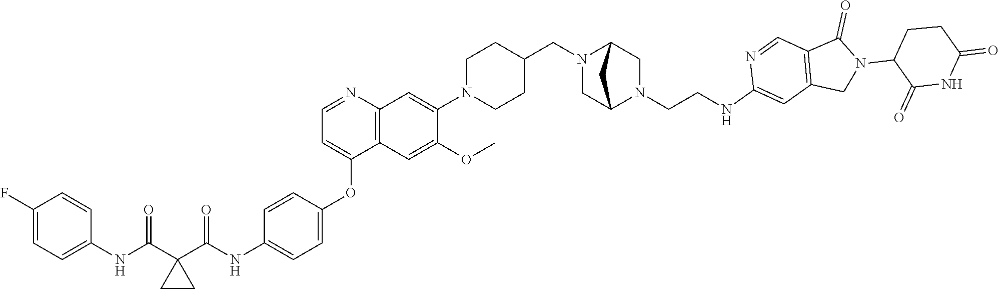 [SMILES: COc1cc2c(Oc3ccc(NC(=O)C4(C(=O)Nc5ccc(F)cc5)CC4)cc3)ccnc2cc1N1CCC(CN2C[C@@H]3C[C@H]2CN3CCNc2cc3c(cn2)C(=O)N(C2CCC(=O)NC2=O)C3)CC1]

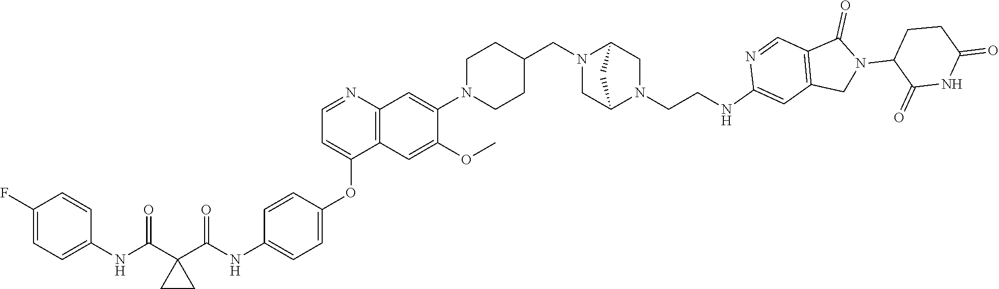 [SMILES: COc1cc2c(Oc3ccc(NC(=O)C4(C(=O)Nc5ccc(F)cc5)CC4)cc3)ccnc2cc1N1CCC(CN2C[C@H]3C[C@@H]2CN3CCNc2cc3c(cn2)C(=O)N(C2CCC(=O)NC2=O)C3)CC1]

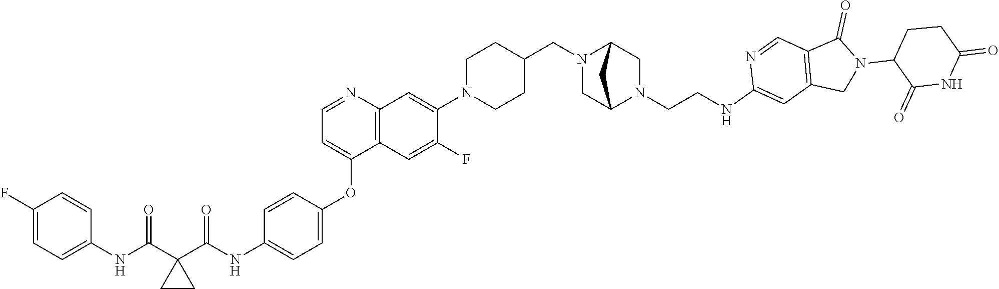 [SMILES: O=C1CCC(N2Cc3cc(NCCN4C[C@@H]5C[C@H]4CN5CC4CCN(c5cc6nccc(Oc7ccc(NC(=O)C8(C(=O)Nc9ccc(F)cc9)CC8)cc7)c6cc5F)CC4)ncc3C2=O)C(=O)N1]

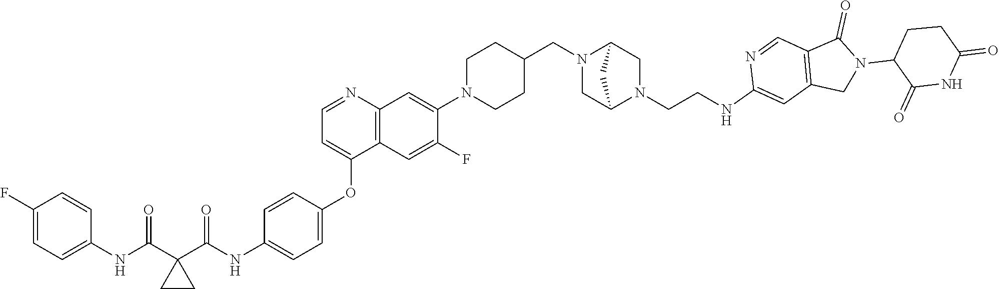 [SMILES: O=C1CCC(N2Cc3cc(NCCN4C[C@H]5C[C@@H]4CN5CC4CCN(c5cc6nccc(Oc7ccc(NC(=O)C8(C(=O)Nc9ccc(F)cc9)CC8)cc7)c6cc5F)CC4)ncc3C2=O)C(=O)N1]

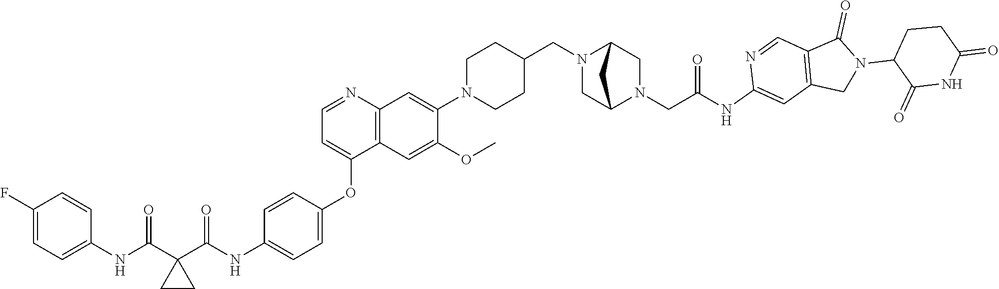 [SMILES: COc1cc2c(Oc3ccc(NC(=O)C4(C(=O)Nc5ccc(F)cc5)CC4)cc3)ccnc2cc1N1CCC(CN2C[C@@H]3C[C@H]2CN3CC(=O)Nc2cc3c(cn2)C(=O)N(C2CCC(=O)NC2=O)C3)CC1]

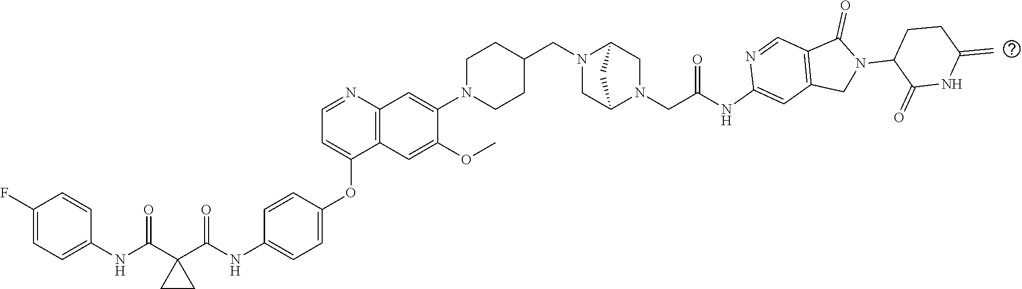 [SMILES: C=C1CCC(N2Cc3cc(NC(=O)CN4C[C@H]5C[C@@H]4CN5CC4CCN(c5cc6nccc(Oc7ccc(NC(=O)C8(C(=O)Nc9ccc(F)cc9)CC8)cc7)c6cc5OC)CC4)ncc3C2=O)C(=O)N1]

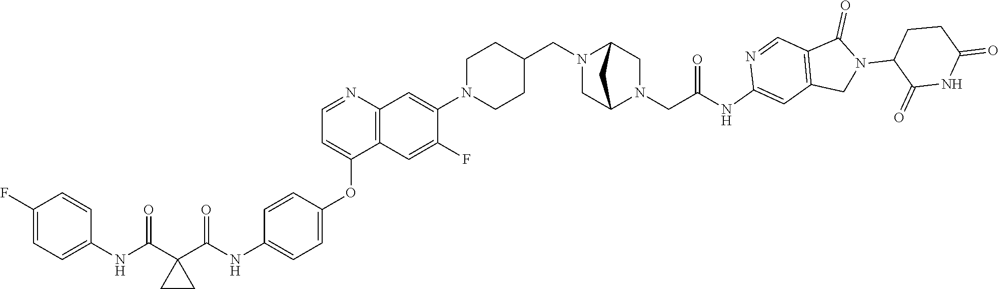 [SMILES: O=C1CCC(N2Cc3cc(NC(=O)CN4C[C@@H]5C[C@H]4CN5CC4CCN(c5cc6nccc(Oc7ccc(NC(=O)C8(C(=O)Nc9ccc(F)cc9)CC8)cc7)c6cc5F)CC4)ncc3C2=O)C(=O)N1]

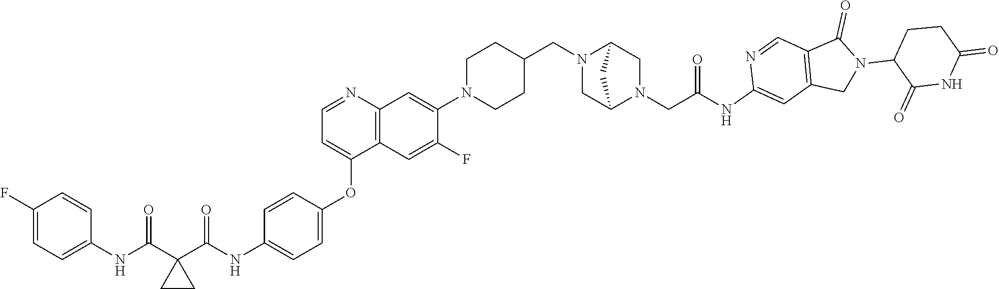 [SMILES: O=C1CCC(N2Cc3cc(NC(=O)CN4C[C@H]5C[C@@H]4CN5CC4CCN(c5cc6nccc(Oc7ccc(NC(=O)C8(C(=O)Nc9ccc(F)cc9)CC8)cc7)c6cc5F)CC4)ncc3C2=O)C(=O)N1]

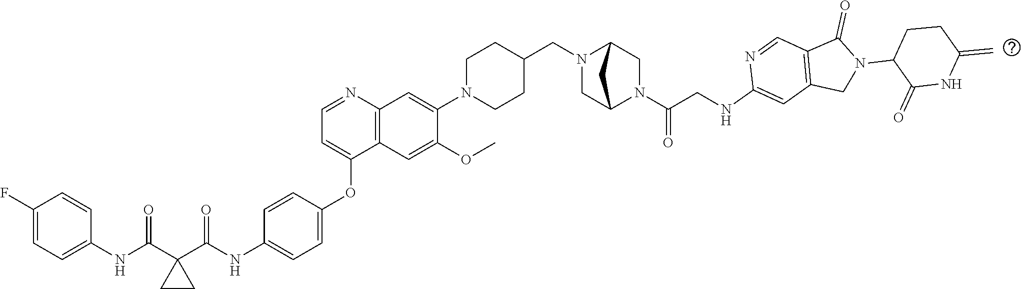 [SMILES: C=C1CCC(N2Cc3cc(NCC(=O)N4C[C@@H]5C[C@H]4CN5CC4CCN(c5cc6nccc(Oc7ccc(NC(=O)C8(C(=O)Nc9ccc(F)cc9)CC8)cc7)c6cc5OC)CC4)ncc3C2=O)C(=O)N1]

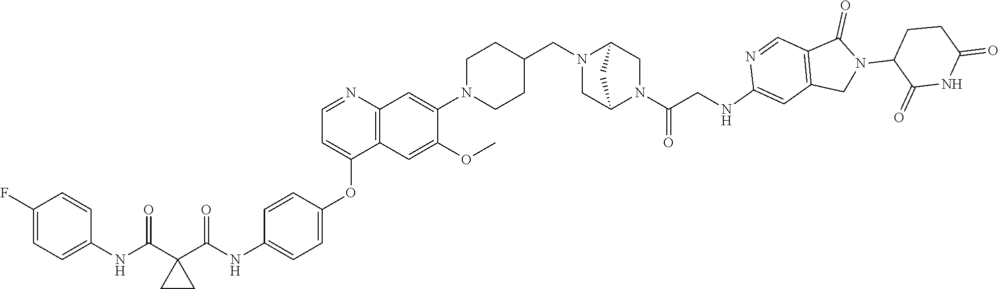 [SMILES: COc1cc2c(Oc3ccc(NC(=O)C4(C(=O)Nc5ccc(F)cc5)CC4)cc3)ccnc2cc1N1CCC(CN2C[C@H]3C[C@@H]2CN3C(=O)CNc2cc3c(cn2)C(=O)N(C2CCC(=O)NC2=O)C3)CC1]

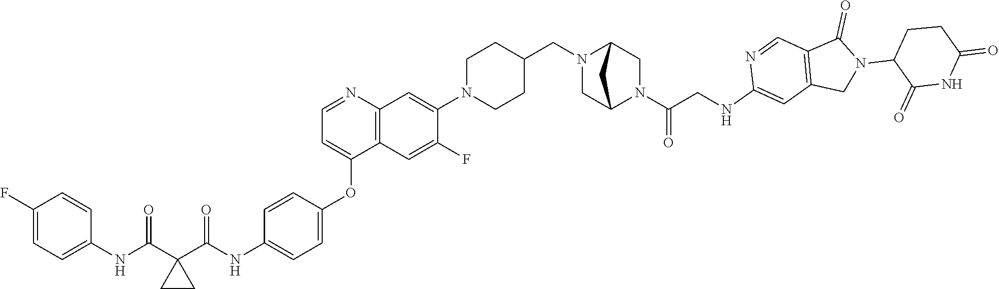 [SMILES: O=C1CCC(N2Cc3cc(NCC(=O)N4C[C@@H]5C[C@H]4CN5CC4CCN(c5cc6nccc(Oc7ccc(NC(=O)C8(C(=O)Nc9ccc(F)cc9)CC8)cc7)c6cc5F)CC4)ncc3C2=O)C(=O)N1]

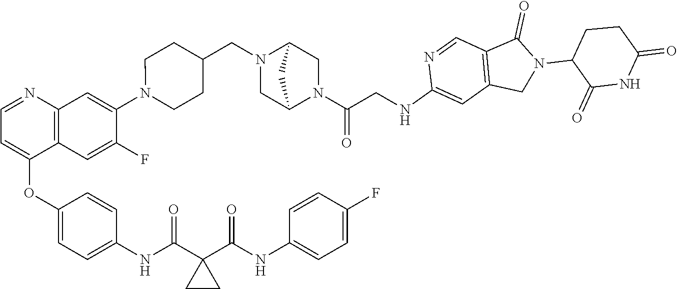 [SMILES: O=C1CCC(N2Cc3cc(NCC(=O)N4C[C@H]5C[C@@H]4CN5CC4CCN(c5cc6nccc(Oc7ccc(NC(=O)C8(C(=O)Nc9ccc(F)cc9)CC8)cc7)c6cc5F)CC4)ncc3C2=O)C(=O)N1]

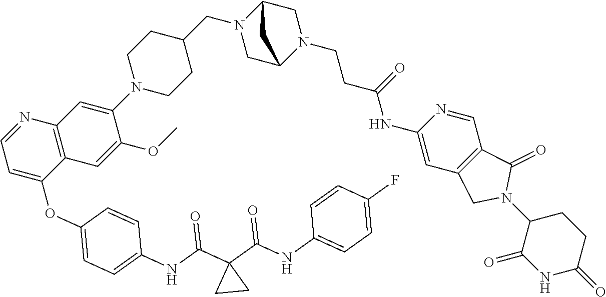 [SMILES: COc1cc2c(Oc3ccc(NC(=O)C4(C(=O)Nc5ccc(F)cc5)CC4)cc3)ccnc2cc1N1CCC(CN2C[C@@H]3C[C@H]2CN3CCC(=O)Nc2cc3c(cn2)C(=O)N(C2CCC(=O)NC2=O)C3)CC1]

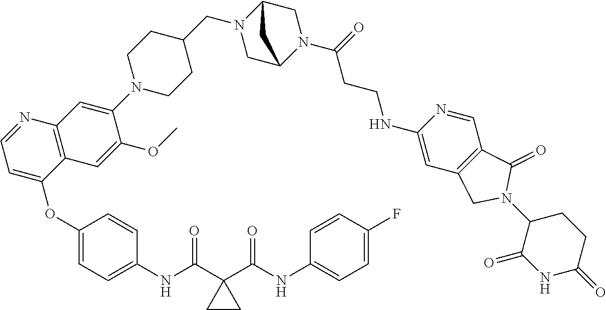 [SMILES: COc1cc2c(Oc3ccc(NC(=O)C4(C(=O)Nc5ccc(F)cc5)CC4)cc3)ccnc2cc1N1CCC(CN2C[C@@H]3C[C@H]2CN3C(=O)CCNc2cc3c(cn2)C(=O)N(C2CCC(=O)NC2=O)C3)CC1]

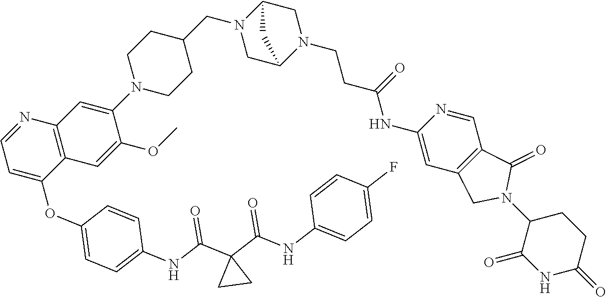 [SMILES: COc1cc2c(Oc3ccc(NC(=O)C4(C(=O)Nc5ccc(F)cc5)CC4)cc3)ccnc2cc1N1CCC(CN2C[C@H]3C[C@@H]2CN3CCC(=O)Nc2cc3c(cn2)C(=O)N(C2CCC(=O)NC2=O)C3)CC1]

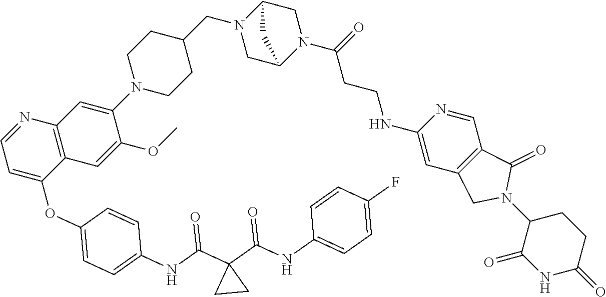 [SMILES: COc1cc2c(Oc3ccc(NC(=O)C4(C(=O)Nc5ccc(F)cc5)CC4)cc3)ccnc2cc1N1CCC(CN2C[C@H]3C[C@@H]2CN3C(=O)CCNc2cc3c(cn2)C(=O)N(C2CCC(=O)NC2=O)C3)CC1]

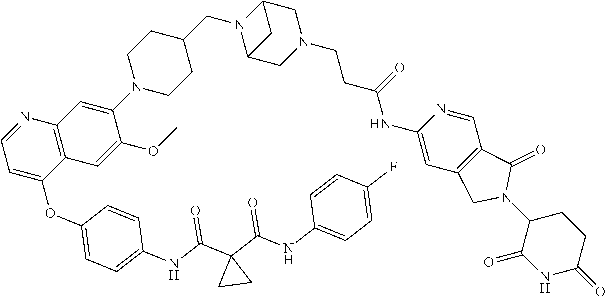 [SMILES: COc1cc2c(Oc3ccc(NC(=O)C4(C(=O)Nc5ccc(F)cc5)CC4)cc3)ccnc2cc1N1CCC(CN2C3CC2CN(CCC(=O)Nc2cc4c(cn2)C(=O)N(C2CCC(=O)NC2=O)C4)C3)CC1]

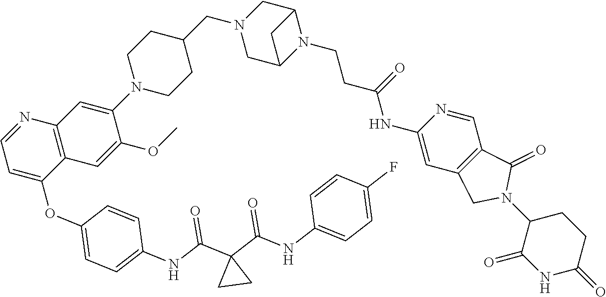 [SMILES: COc1cc2c(Oc3ccc(NC(=O)C4(C(=O)Nc5ccc(F)cc5)CC4)cc3)ccnc2cc1N1CCC(CN2CC3CC(C2)N3CCC(=O)Nc2cc3c(cn2)C(=O)N(C2CCC(=O)NC2=O)C3)CC1]

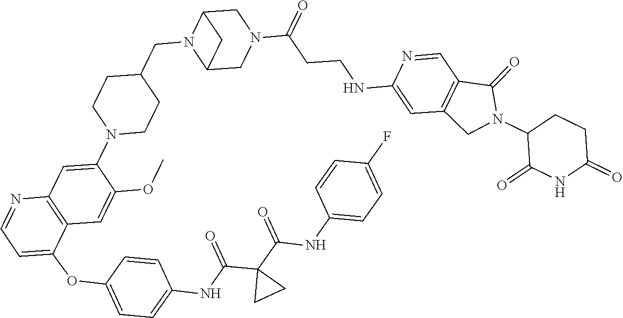 [SMILES: COc1cc2c(Oc3ccc(NC(=O)C4(C(=O)Nc5ccc(F)cc5)CC4)cc3)ccnc2cc1N1CCC(CN2C3CC2CN(C(=O)CCNc2cc4c(cn2)C(=O)N(C2CCC(=O)NC2=O)C4)C3)CC1]

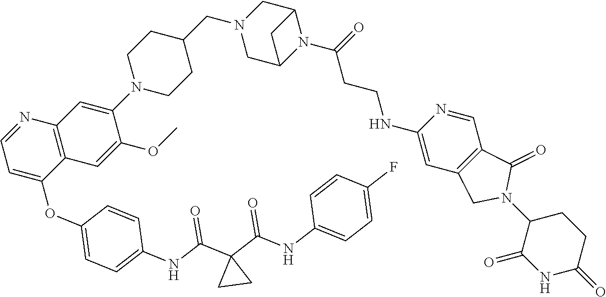 [SMILES: COc1cc2c(Oc3ccc(NC(=O)C4(C(=O)Nc5ccc(F)cc5)CC4)cc3)ccnc2cc1N1CCC(CN2CC3CC(C2)N3C(=O)CCNc2cc3c(cn2)C(=O)N(C2CCC(=O)NC2=O)C3)CC1]

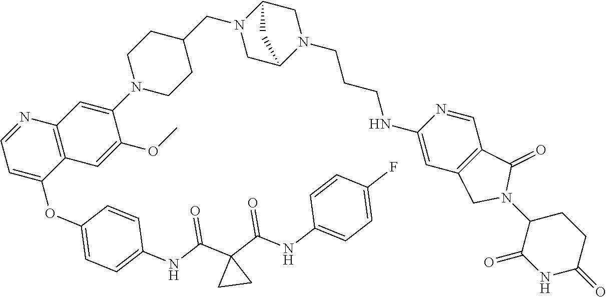 [SMILES: COc1cc2c(Oc3ccc(NC(=O)C4(C(=O)Nc5ccc(F)cc5)CC4)cc3)ccnc2cc1N1CCC(CN2C[C@H]3C[C@@H]2CN3CCCNc2cc3c(cn2)C(=O)N(C2CCC(=O)NC2=O)C3)CC1]

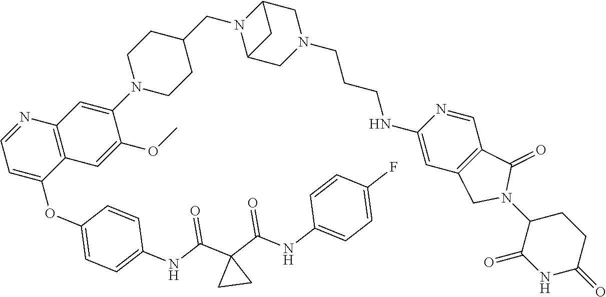 [SMILES: COc1cc2c(Oc3ccc(NC(=O)C4(C(=O)Nc5ccc(F)cc5)CC4)cc3)ccnc2cc1N1CCC(CN2C3CC2CN(CCCNc2cc4c(cn2)C(=O)N(C2CCC(=O)NC2=O)C4)C3)CC1]

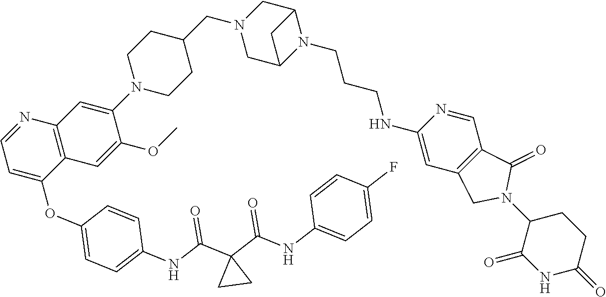 [SMILES: COc1cc2c(Oc3ccc(NC(=O)C4(C(=O)Nc5ccc(F)cc5)CC4)cc3)ccnc2cc1N1CCC(CN2CC3CC(C2)N3CCCNc2cc3c(cn2)C(=O)N(C2CCC(=O)NC2=O)C3)CC1]

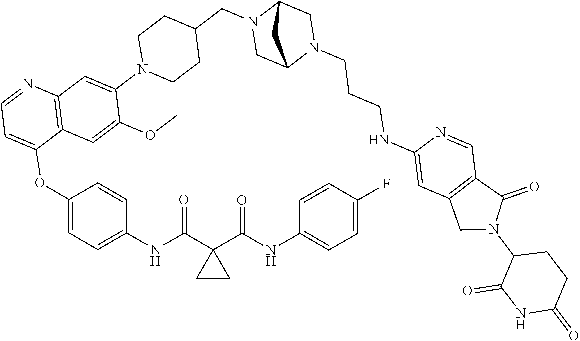 [SMILES: COc1cc2c(Oc3ccc(NC(=O)C4(C(=O)Nc5ccc(F)cc5)CC4)cc3)ccnc2cc1N1CCC(CN2C[C@@H]3C[C@H]2CN3CCCNc2cc3c(cn2)C(=O)N(C2CCC(=O)NC2=O)C3)CC1]

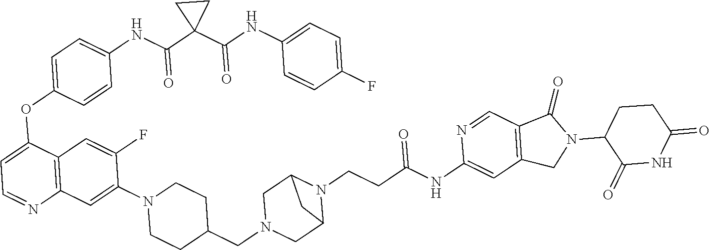 [SMILES: O=C1CCC(N2Cc3cc(NC(=O)CCN4C5CC4CN(CC4CCN(c6cc7nccc(Oc8ccc(NC(=O)C9(C(=O)Nc%10ccc(F)cc%10)CC9)cc8)c7cc6F)CC4)C5)ncc3C2=O)C(=O)N1]

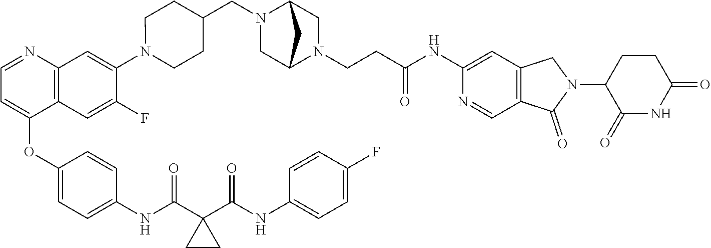 [SMILES: O=C1CCC(N2Cc3cc(NC(=O)CCN4C[C@@H]5C[C@H]4CN5CC4CCN(c5cc6nccc(Oc7ccc(NC(=O)C8(C(=O)Nc9ccc(F)cc9)CC8)cc7)c6cc5F)CC4)ncc3C2=O)C(=O)N1]

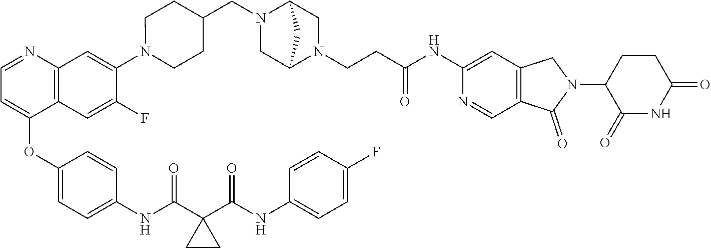 [SMILES: O=C1CCC(N2Cc3cc(NC(=O)CCN4C[C@H]5C[C@@H]4CN5CC4CCN(c5cc6nccc(Oc7ccc(NC(=O)C8(C(=O)Nc9ccc(F)cc9)CC8)cc7)c6cc5F)CC4)ncc3C2=O)C(=O)N1]

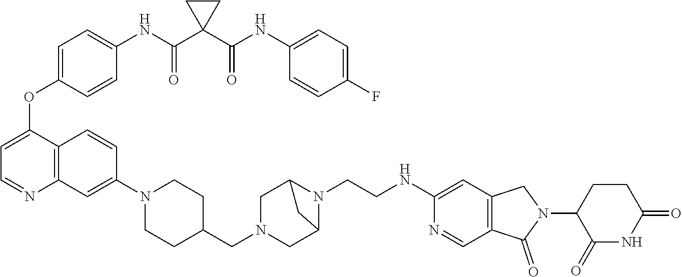 [SMILES: O=C1CCC(N2Cc3cc(NCCN4C5CC4CN(CC4CCN(c6ccc7c(Oc8ccc(NC(=O)C9(C(=O)Nc%10ccc(F)cc%10)CC9)cc8)ccnc7c6)CC4)C5)ncc3C2=O)C(=O)N1]

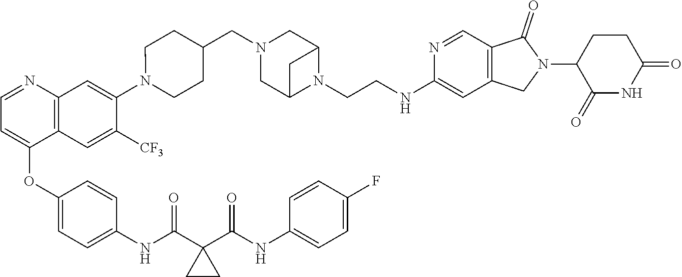 [SMILES: O=C1CCC(N2Cc3cc(NCCN4C5CC4CN(CC4CCN(c6cc7nccc(Oc8ccc(NC(=O)C9(C(=O)Nc%10ccc(F)cc%10)CC9)cc8)c7cc6C(F)(F)F)CC4)C5)ncc3C2=O)C(=O)N1]

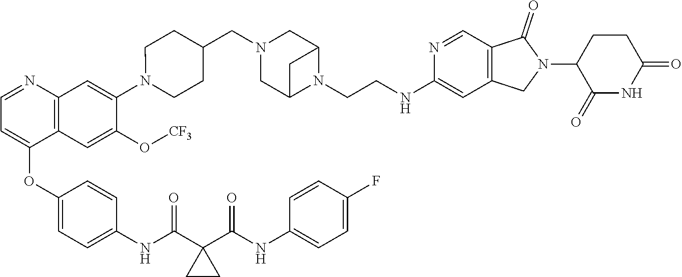 [SMILES: O=C1CCC(N2Cc3cc(NCCN4C5CC4CN(CC4CCN(c6cc7nccc(Oc8ccc(NC(=O)C9(C(=O)Nc%10ccc(F)cc%10)CC9)cc8)c7cc6OC(F)(F)F)CC4)C5)ncc3C2=O)C(=O)N1]

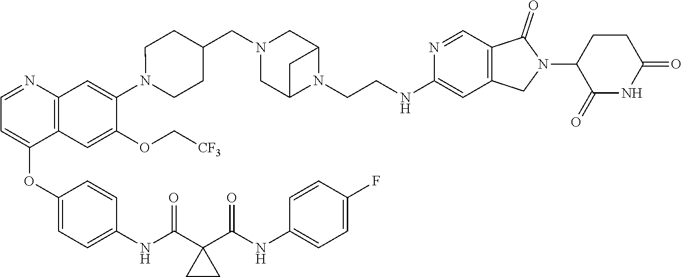 [SMILES: O=C1CCC(N2Cc3cc(NCCN4C5CC4CN(CC4CCN(c6cc7nccc(Oc8ccc(NC(=O)C9(C(=O)Nc%10ccc(F)cc%10)CC9)cc8)c7cc6OCC(F)(F)F)CC4)C5)ncc3C2=O)C(=O)N1]

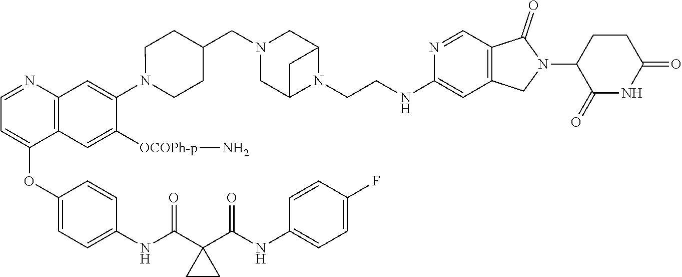

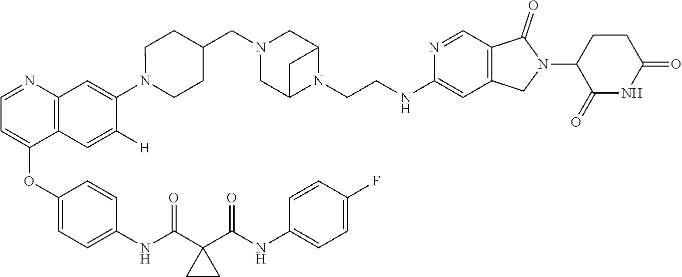 [SMILES: O=C1CCC(N2Cc3cc(NCCN4C5CC4CN(CC4CCN(c6ccc7c(Oc8ccc(NC(=O)C9(C(=O)Nc%10ccc(F)cc%10)CC9)cc8)ccnc7c6)CC4)C5)ncc3C2=O)C(=O)N1]

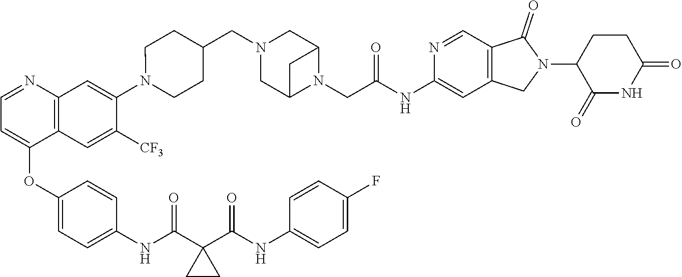 [SMILES: O=C1CCC(N2Cc3cc(NC(=O)CN4C5CC4CN(CC4CCN(c6cc7nccc(Oc8ccc(NC(=O)C9(C(=O)Nc%10ccc(F)cc%10)CC9)cc8)c7cc6C(F)(F)F)CC4)C5)ncc3C2=O)C(=O)N1]

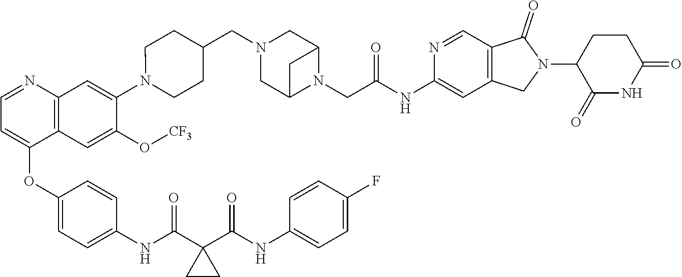 [SMILES: O=C1CCC(N2Cc3cc(NC(=O)CN4C5CC4CN(CC4CCN(c6cc7nccc(Oc8ccc(NC(=O)C9(C(=O)Nc%10ccc(F)cc%10)CC9)cc8)c7cc6OC(F)(F)F)CC4)C5)ncc3C2=O)C(=O)N1]

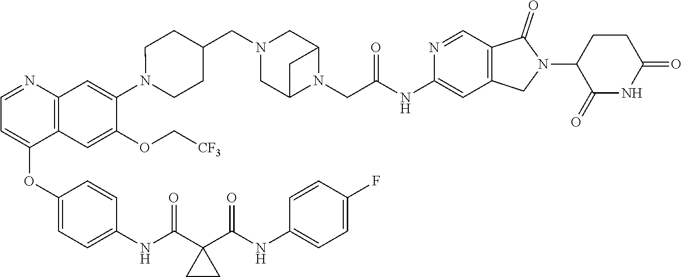 [SMILES: O=C1CCC(N2Cc3cc(NC(=O)CN4C5CC4CN(CC4CCN(c6cc7nccc(Oc8ccc(NC(=O)C9(C(=O)Nc%10ccc(F)cc%10)CC9)cc8)c7cc6OCC(F)(F)F)CC4)C5)ncc3C2=O)C(=O)N1]

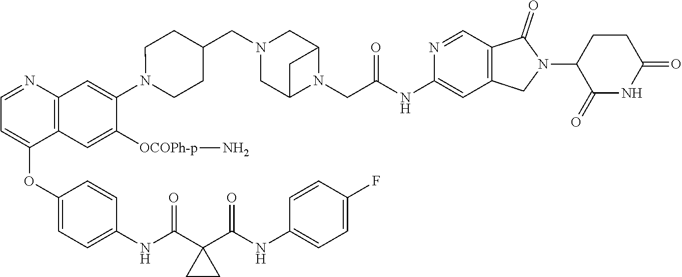

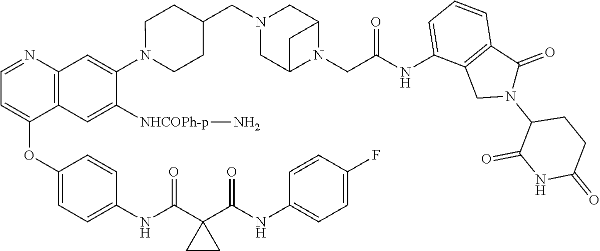

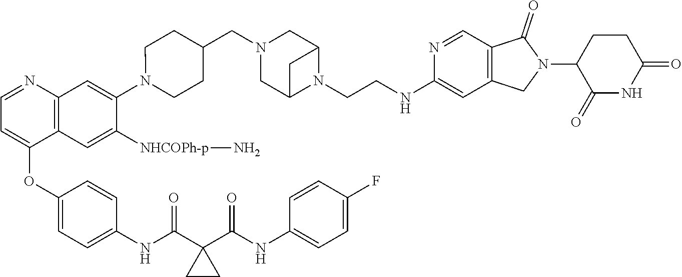

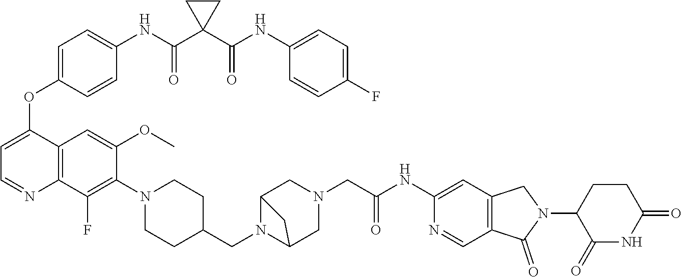 [SMILES: COc1cc2c(Oc3ccc(NC(=O)C4(C(=O)Nc5ccc(F)cc5)CC4)cc3)ccnc2c(F)c1N1CCC(CN2C3CC2CN(CC(=O)Nc2cc4c(cn2)C(=O)N(C2CCC(=O)NC2=O)C4)C3)CC1]

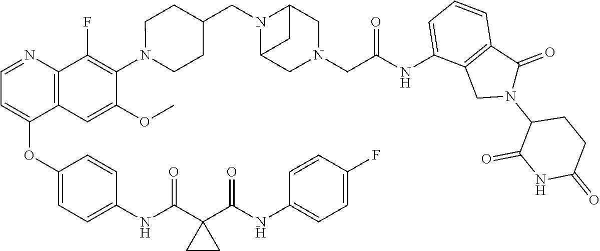 [SMILES: COc1cc2c(Oc3ccc(NC(=O)C4(C(=O)Nc5ccc(F)cc5)CC4)cc3)ccnc2c(F)c1N1CCC(CN2C3CC2CN(CC(=O)Nc2cccc4c2CN(C2CCC(=O)NC2=O)C4=O)C3)CC1]

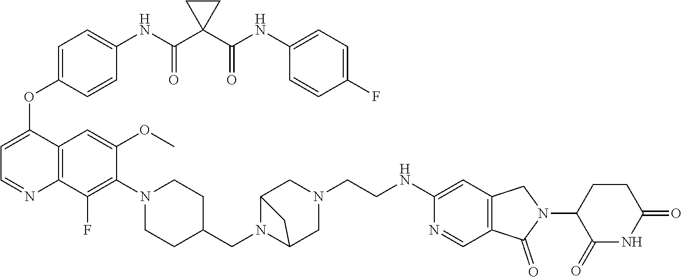 [SMILES: COc1cc2c(Oc3ccc(NC(=O)C4(C(=O)Nc5ccc(F)cc5)CC4)cc3)ccnc2c(F)c1N1CCC(CN2C3CC2CN(CCNc2cc4c(cn2)C(=O)N(C2CCC(=O)NC2=O)C4)C3)CC1]

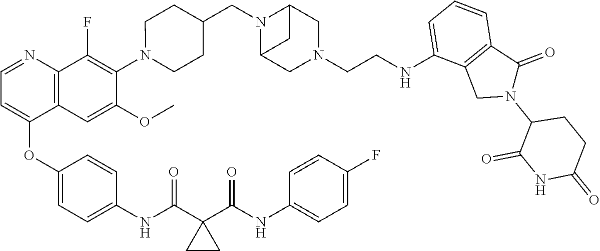 [SMILES: COc1cc2c(Oc3ccc(NC(=O)C4(C(=O)Nc5ccc(F)cc5)CC4)cc3)ccnc2c(F)c1N1CCC(CN2C3CC2CN(CCNc2cccc4c2CN(C2CCC(=O)NC2=O)C4=O)C3)CC1]

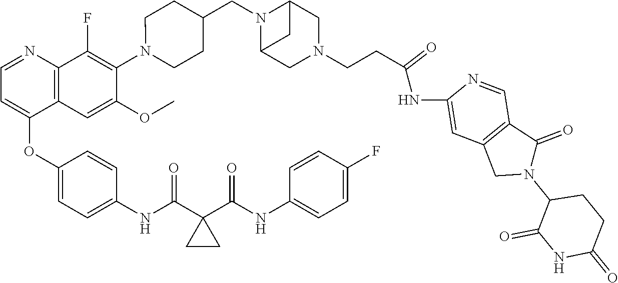 [SMILES: COc1cc2c(Oc3ccc(NC(=O)C4(C(=O)Nc5ccc(F)cc5)CC4)cc3)ccnc2c(F)c1N1CCC(CN2C3CC2CN(CCC(=O)Nc2cc4c(cn2)C(=O)N(C2CCC(=O)NC2=O)C4)C3)CC1]

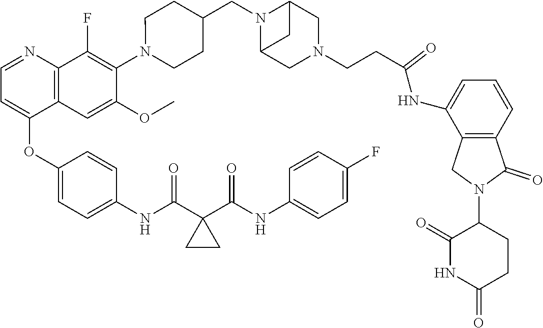 [SMILES: COc1cc2c(Oc3ccc(NC(=O)C4(C(=O)Nc5ccc(F)cc5)CC4)cc3)ccnc2c(F)c1N1CCC(CN2C3CC2CN(CCC(=O)Nc2cccc4c2CN(C2CCC(=O)NC2=O)C4=O)C3)CC1]

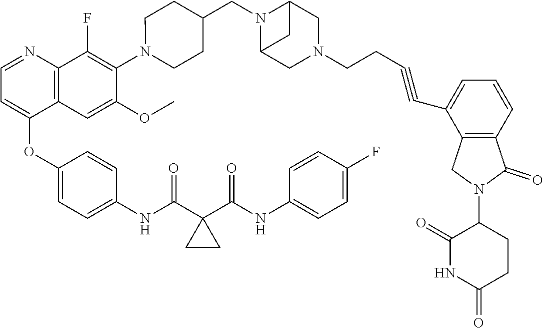 [SMILES: COc1cc2c(Oc3ccc(NC(=O)C4(C(=O)Nc5ccc(F)cc5)CC4)cc3)ccnc2c(F)c1N1CCC(CN2C3CC2CN(CCC#Cc2cccc4c2CN(C2CCC(=O)NC2=O)C4=O)C3)CC1]

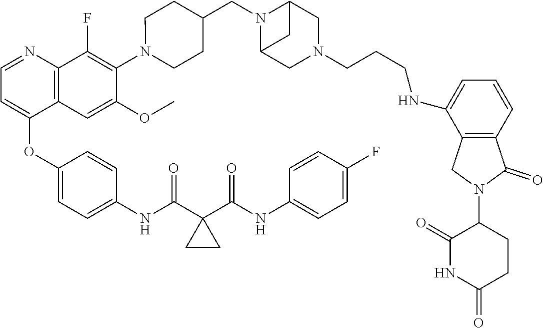 [SMILES: COc1cc2c(Oc3ccc(NC(=O)C4(C(=O)Nc5ccc(F)cc5)CC4)cc3)ccnc2c(F)c1N1CCC(CN2C3CC2CN(CCCNc2cccc4c2CN(C2CCC(=O)NC2=O)C4=O)C3)CC1]

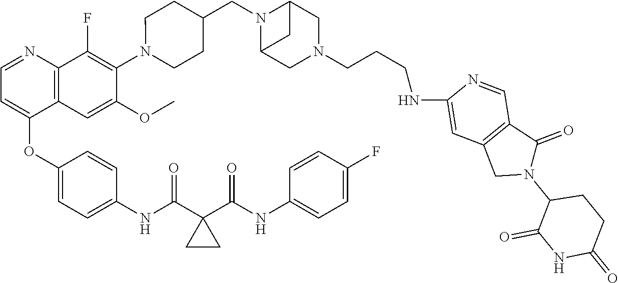 [SMILES: COc1cc2c(Oc3ccc(NC(=O)C4(C(=O)Nc5ccc(F)cc5)CC4)cc3)ccnc2c(F)c1N1CCC(CN2C3CC2CN(CCCNc2cc4c(cn2)C(=O)N(C2CCC(=O)NC2=O)C4)C3)CC1]

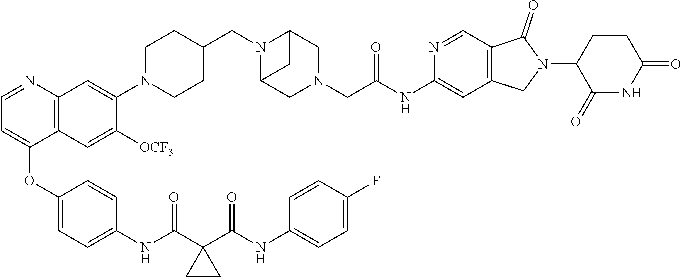 [SMILES: O=C1CCC(N2Cc3cc(NC(=O)CN4CC5CC(C4)N5CC4CCN(c5cc6nccc(Oc7ccc(NC(=O)C8(C(=O)Nc9ccc(F)cc9)CC8)cc7)c6cc5OC(F)(F)F)CC4)ncc3C2=O)C(=O)N1]

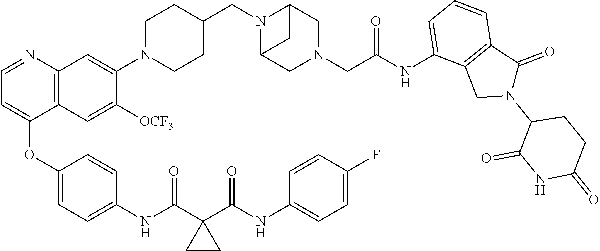 [SMILES: O=C1CCC(N2Cc3c(NC(=O)CN4CC5CC(C4)N5CC4CCN(c5cc6nccc(Oc7ccc(NC(=O)C8(C(=O)Nc9ccc(F)cc9)CC8)cc7)c6cc5OC(F)(F)F)CC4)cccc3C2=O)C(=O)N1]

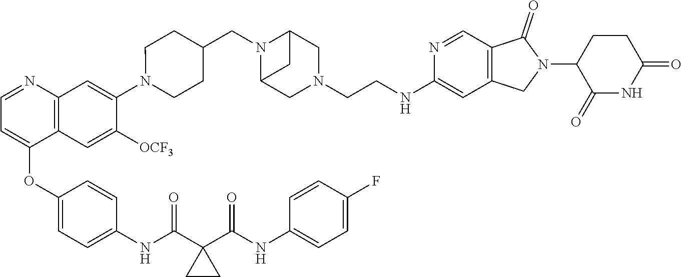 [SMILES: O=C1CCC(N2Cc3cc(NCCN4CC5CC(C4)N5CC4CCN(c5cc6nccc(Oc7ccc(NC(=O)C8(C(=O)Nc9ccc(F)cc9)CC8)cc7)c6cc5OC(F)(F)F)CC4)ncc3C2=O)C(=O)N1]

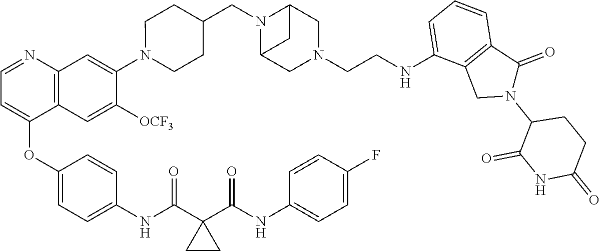 [SMILES: O=C1CCC(N2Cc3c(NCCN4CC5CC(C4)N5CC4CCN(c5cc6nccc(Oc7ccc(NC(=O)C8(C(=O)Nc9ccc(F)cc9)CC8)cc7)c6cc5OC(F)(F)F)CC4)cccc3C2=O)C(=O)N1]

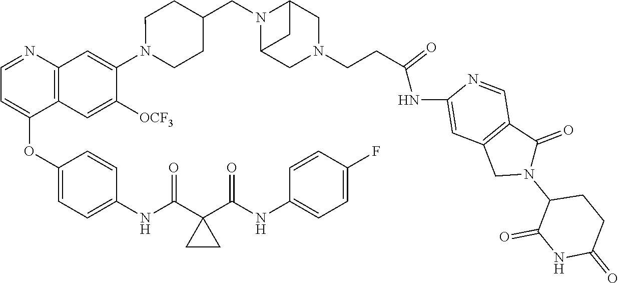 [SMILES: O=C1CCC(N2Cc3cc(NC(=O)CCN4CC5CC(C4)N5CC4CCN(c5cc6nccc(Oc7ccc(NC(=O)C8(C(=O)Nc9ccc(F)cc9)CC8)cc7)c6cc5OC(F)(F)F)CC4)ncc3C2=O)C(=O)N1]

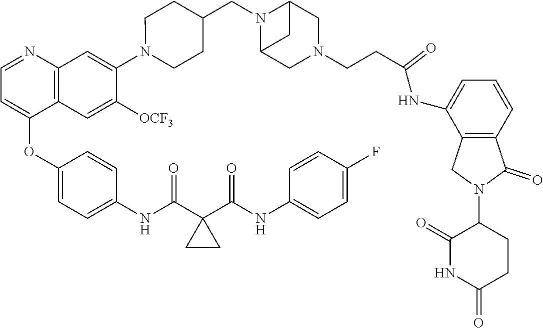 [SMILES: O=C1CCC(N2Cc3c(NC(=O)CCN4CC5CC(C4)N5CC4CCN(c5cc6nccc(Oc7ccc(NC(=O)C8(C(=O)Nc9ccc(F)cc9)CC8)cc7)c6cc5OC(F)(F)F)CC4)cccc3C2=O)C(=O)N1]

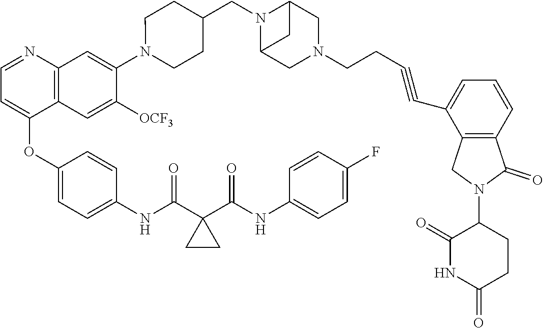 [SMILES: O=C1CCC(N2Cc3c(C#CCCN4CC5CC(C4)N5CC4CCN(c5cc6nccc(Oc7ccc(NC(=O)C8(C(=O)Nc9ccc(F)cc9)CC8)cc7)c6cc5OC(F)(F)F)CC4)cccc3C2=O)C(=O)N1]

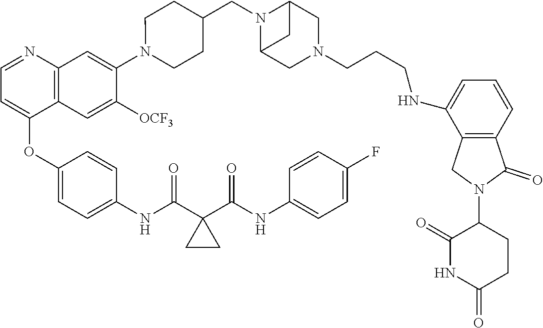 [SMILES: O=C1CCC(N2Cc3c(NCCCN4CC5CC(C4)N5CC4CCN(c5cc6nccc(Oc7ccc(NC(=O)C8(C(=O)Nc9ccc(F)cc9)CC8)cc7)c6cc5OC(F)(F)F)CC4)cccc3C2=O)C(=O)N1]

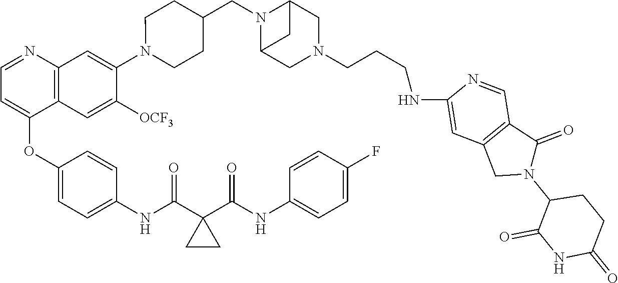 [SMILES: O=C1CCC(N2Cc3cc(NCCCN4CC5CC(C4)N5CC4CCN(c5cc6nccc(Oc7ccc(NC(=O)C8(C(=O)Nc9ccc(F)cc9)CC8)cc7)c6cc5OC(F)(F)F)CC4)ncc3C2=O)C(=O)N1]

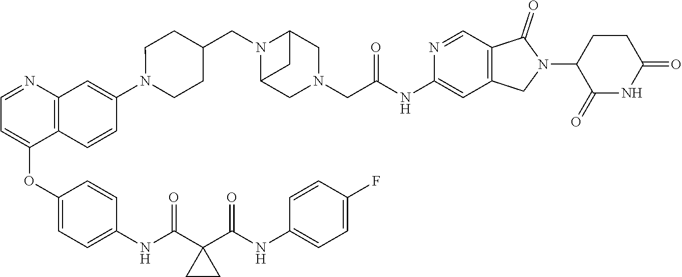 [SMILES: O=C1CCC(N2Cc3cc(NC(=O)CN4CC5CC(C4)N5CC4CCN(c5ccc6c(Oc7ccc(NC(=O)C8(C(=O)Nc9ccc(F)cc9)CC8)cc7)ccnc6c5)CC4)ncc3C2=O)C(=O)N1]

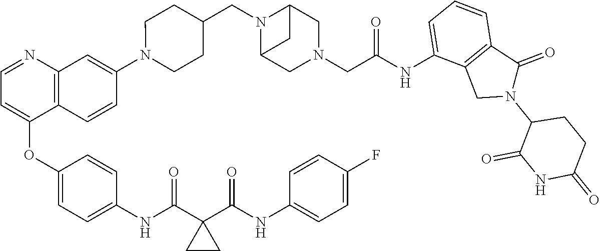 [SMILES: O=C1CCC(N2Cc3c(NC(=O)CN4CC5CC(C4)N5CC4CCN(c5ccc6c(Oc7ccc(NC(=O)C8(C(=O)Nc9ccc(F)cc9)CC8)cc7)ccnc6c5)CC4)cccc3C2=O)C(=O)N1]

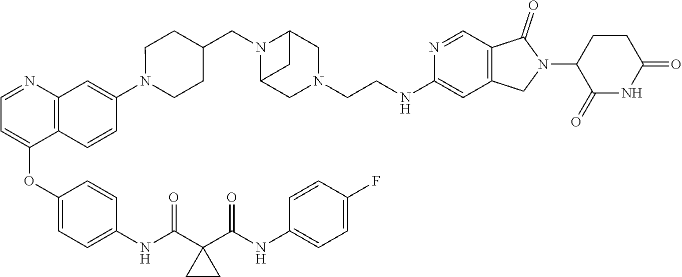 [SMILES: O=C1CCC(N2Cc3cc(NCCN4CC5CC(C4)N5CC4CCN(c5ccc6c(Oc7ccc(NC(=O)C8(C(=O)Nc9ccc(F)cc9)CC8)cc7)ccnc6c5)CC4)ncc3C2=O)C(=O)N1]

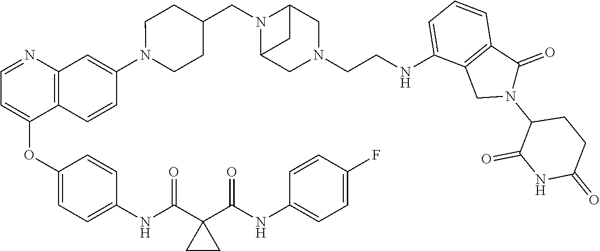 [SMILES: O=C1CCC(N2Cc3c(NCCN4CC5CC(C4)N5CC4CCN(c5ccc6c(Oc7ccc(NC(=O)C8(C(=O)Nc9ccc(F)cc9)CC8)cc7)ccnc6c5)CC4)cccc3C2=O)C(=O)N1]

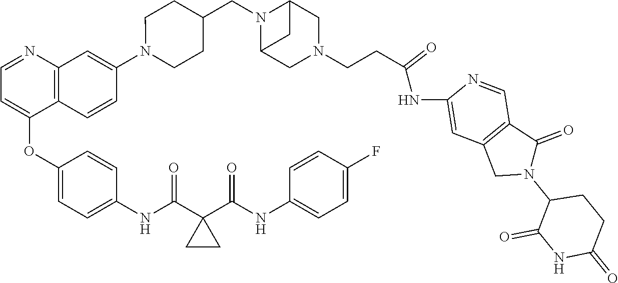 [SMILES: O=C1CCC(N2Cc3cc(NC(=O)CCN4CC5CC(C4)N5CC4CCN(c5ccc6c(Oc7ccc(NC(=O)C8(C(=O)Nc9ccc(F)cc9)CC8)cc7)ccnc6c5)CC4)ncc3C2=O)C(=O)N1]

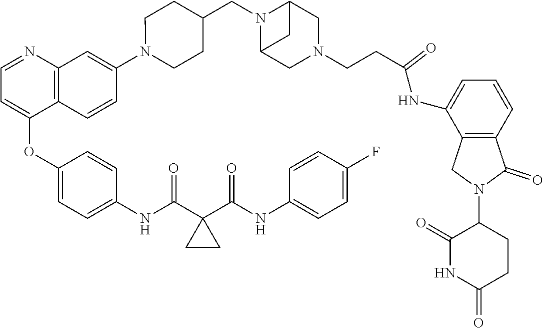 [SMILES: O=C1CCC(N2Cc3c(NC(=O)CCN4CC5CC(C4)N5CC4CCN(c5ccc6c(Oc7ccc(NC(=O)C8(C(=O)Nc9ccc(F)cc9)CC8)cc7)ccnc6c5)CC4)cccc3C2=O)C(=O)N1]

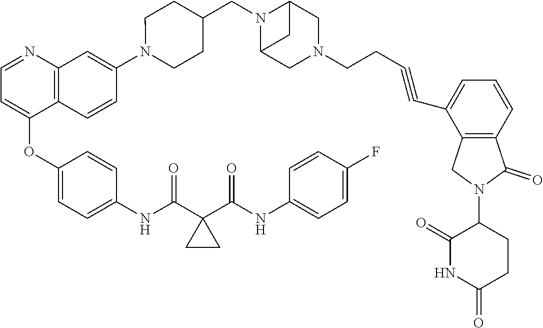 [SMILES: O=C1CCC(N2Cc3c(C#CCCN4CC5CC(C4)N5CC4CCN(c5ccc6c(Oc7ccc(NC(=O)C8(C(=O)Nc9ccc(F)cc9)CC8)cc7)ccnc6c5)CC4)cccc3C2=O)C(=O)N1]

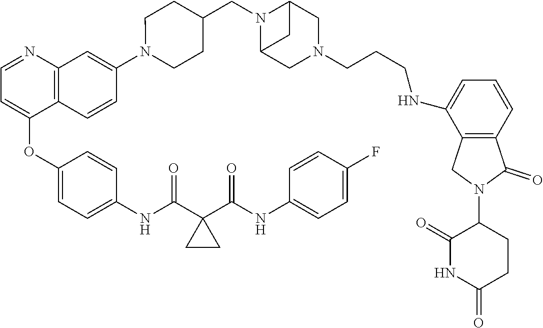 [SMILES: O=C1CCC(N2Cc3c(NCCCN4CC5CC(C4)N5CC4CCN(c5ccc6c(Oc7ccc(NC(=O)C8(C(=O)Nc9ccc(F)cc9)CC8)cc7)ccnc6c5)CC4)cccc3C2=O)C(=O)N1]

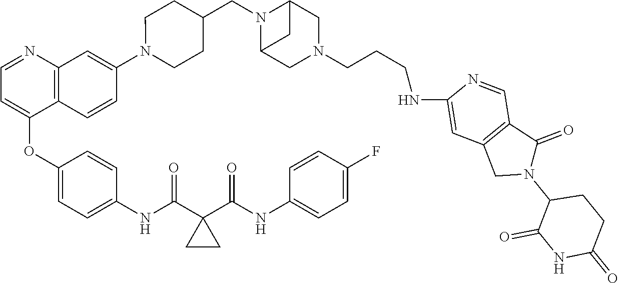 [SMILES: O=C1CCC(N2Cc3cc(NCCCN4CC5CC(C4)N5CC4CCN(c5ccc6c(Oc7ccc(NC(=O)C8(C(=O)Nc9ccc(F)cc9)CC8)cc7)ccnc6c5)CC4)ncc3C2=O)C(=O)N1]

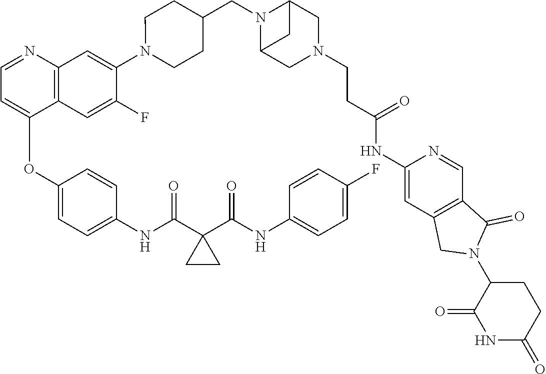 [SMILES: O=C1CCC(N2Cc3cc(NC(=O)CCN4CC5CC(C4)N5CC4CCN(c5cc6nccc(Oc7ccc(NC(=O)C8(C(=O)Nc9ccc(F)cc9)CC8)cc7)c6cc5F)CC4)ncc3C2=O)C(=O)N1]

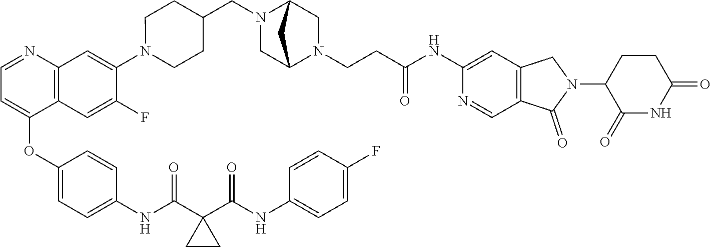 [SMILES: O=C1CCC(N2Cc3cc(NC(=O)CCN4C[C@@H]5C[C@H]4CN5CC4CCN(c5cc6nccc(Oc7ccc(NC(=O)C8(C(=O)Nc9ccc(F)cc9)CC8)cc7)c6cc5F)CC4)ncc3C2=O)C(=O)N1]

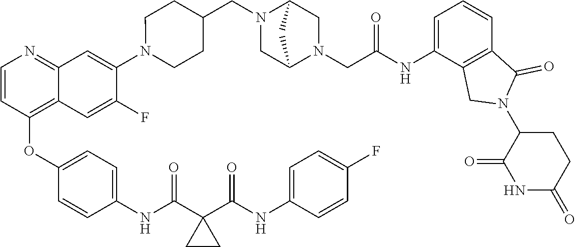 [SMILES: O=C1CCC(N2Cc3c(NC(=O)CN4C[C@H]5C[C@@H]4CN5CC4CCN(c5cc6nccc(Oc7ccc(NC(=O)C8(C(=O)Nc9ccc(F)cc9)CC8)cc7)c6cc5F)CC4)cccc3C2=O)C(=O)N1]

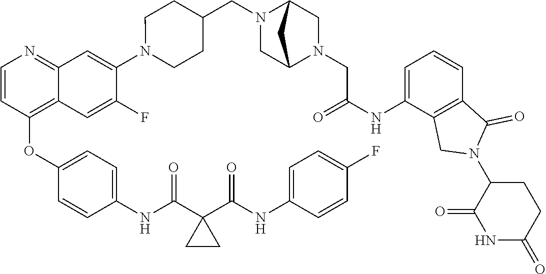 [SMILES: O=C1CCC(N2Cc3c(NC(=O)CN4C[C@@H]5C[C@H]4CN5CC4CCN(c5cc6nccc(Oc7ccc(NC(=O)C8(C(=O)Nc9ccc(F)cc9)CC8)cc7)c6cc5F)CC4)cccc3C2=O)C(=O)N1]

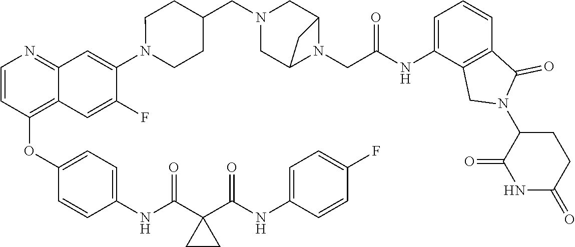 [SMILES: O=C1CCC(N2Cc3c(NC(=O)CN4C5CC4CN(CC4CCN(c6cc7nccc(Oc8ccc(NC(=O)C9(C(=O)Nc%10ccc(F)cc%10)CC9)cc8)c7cc6F)CC4)C5)cccc3C2=O)C(=O)N1]

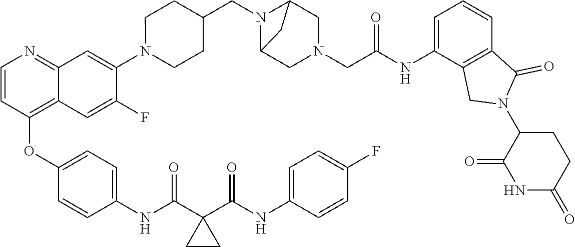 [SMILES: O=C1CCC(N2Cc3c(NC(=O)CN4CC5CC(C4)N5CC4CCN(c5cc6nccc(Oc7ccc(NC(=O)C8(C(=O)Nc9ccc(F)cc9)CC8)cc7)c6cc5F)CC4)cccc3C2=O)C(=O)N1]

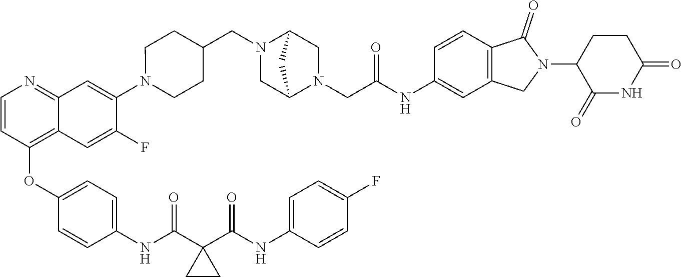 [SMILES: O=C1CCC(N2Cc3cc(NC(=O)CN4C[C@H]5C[C@@H]4CN5CC4CCN(c5cc6nccc(Oc7ccc(NC(=O)C8(C(=O)Nc9ccc(F)cc9)CC8)cc7)c6cc5F)CC4)ccc3C2=O)C(=O)N1]

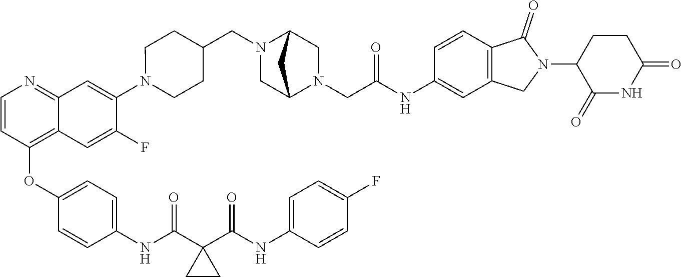 [SMILES: O=C1CCC(N2Cc3cc(NC(=O)CN4C[C@@H]5C[C@H]4CN5CC4CCN(c5cc6nccc(Oc7ccc(NC(=O)C8(C(=O)Nc9ccc(F)cc9)CC8)cc7)c6cc5F)CC4)ccc3C2=O)C(=O)N1]

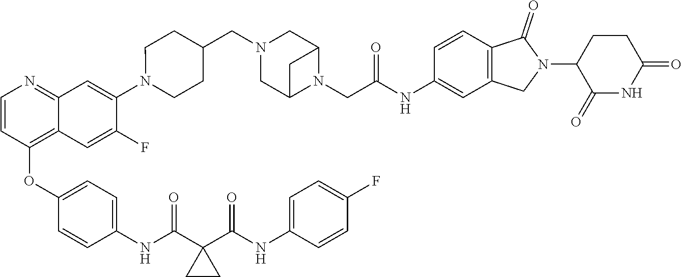 [SMILES: O=C1CCC(N2Cc3cc(NC(=O)CN4C5CC4CN(CC4CCN(c6cc7nccc(Oc8ccc(NC(=O)C9(C(=O)Nc%10ccc(F)cc%10)CC9)cc8)c7cc6F)CC4)C5)ccc3C2=O)C(=O)N1]

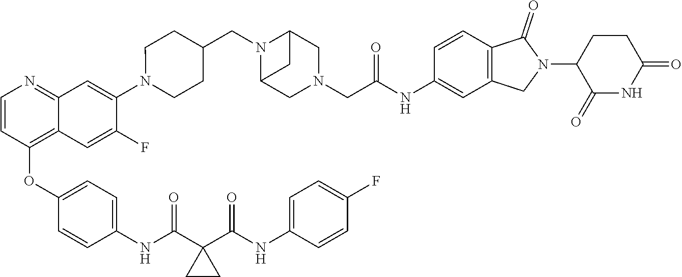 [SMILES: O=C1CCC(N2Cc3cc(NC(=O)CN4CC5CC(C4)N5CC4CCN(c5cc6nccc(Oc7ccc(NC(=O)C8(C(=O)Nc9ccc(F)cc9)CC8)cc7)c6cc5F)CC4)ccc3C2=O)C(=O)N1]

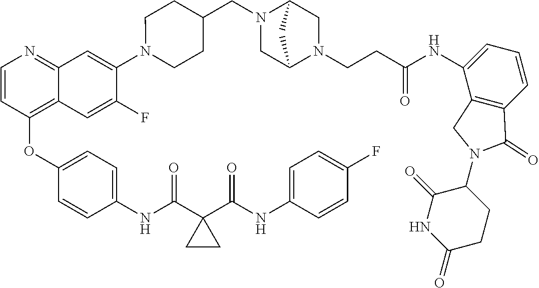 [SMILES: O=C1CCC(N2Cc3c(NC(=O)CCN4C[C@H]5C[C@@H]4CN5CC4CCN(c5cc6nccc(Oc7ccc(NC(=O)C8(C(=O)Nc9ccc(F)cc9)CC8)cc7)c6cc5F)CC4)cccc3C2=O)C(=O)N1]

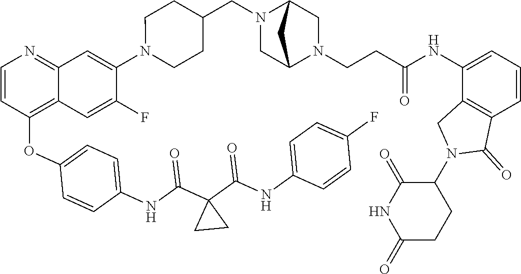 [SMILES: O=C1CCC(N2Cc3c(NC(=O)CCN4C[C@@H]5C[C@H]4CN5CC4CCN(c5cc6nccc(Oc7ccc(NC(=O)C8(C(=O)Nc9ccc(F)cc9)CC8)cc7)c6cc5F)CC4)cccc3C2=O)C(=O)N1]

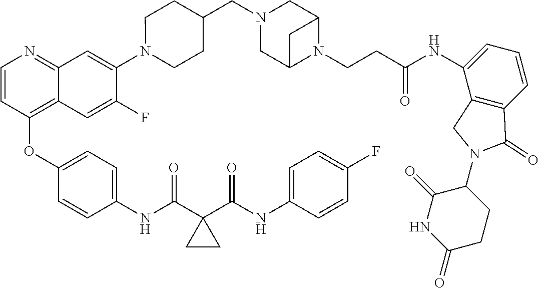 [SMILES: O=C1CCC(N2Cc3c(NC(=O)CCN4C5CC4CN(CC4CCN(c6cc7nccc(Oc8ccc(NC(=O)C9(C(=O)Nc%10ccc(F)cc%10)CC9)cc8)c7cc6F)CC4)C5)cccc3C2=O)C(=O)N1]

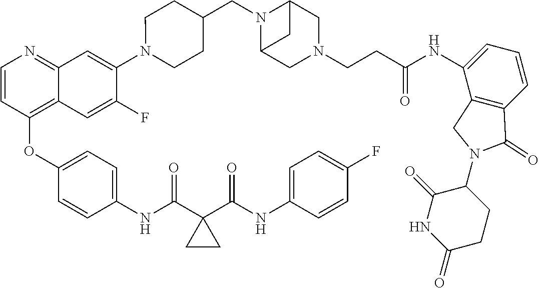 [SMILES: O=C1CCC(N2Cc3c(NC(=O)CCN4CC5CC(C4)N5CC4CCN(c5cc6nccc(Oc7ccc(NC(=O)C8(C(=O)Nc9ccc(F)cc9)CC8)cc7)c6cc5F)CC4)cccc3C2=O)C(=O)N1]

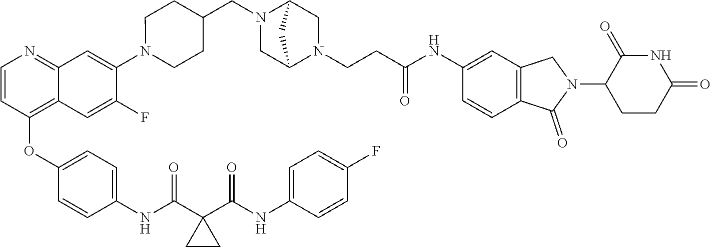 [SMILES: O=C1CCC(N2Cc3cc(NC(=O)CCN4C[C@H]5C[C@@H]4CN5CC4CCN(c5cc6nccc(Oc7ccc(NC(=O)C8(C(=O)Nc9ccc(F)cc9)CC8)cc7)c6cc5F)CC4)ccc3C2=O)C(=O)N1]

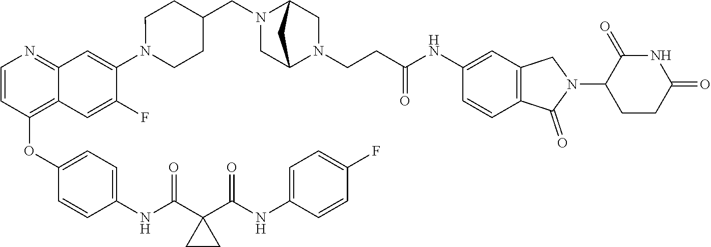 [SMILES: O=C1CCC(N2Cc3cc(NC(=O)CCN4C[C@@H]5C[C@H]4CN5CC4CCN(c5cc6nccc(Oc7ccc(NC(=O)C8(C(=O)Nc9ccc(F)cc9)CC8)cc7)c6cc5F)CC4)ccc3C2=O)C(=O)N1]

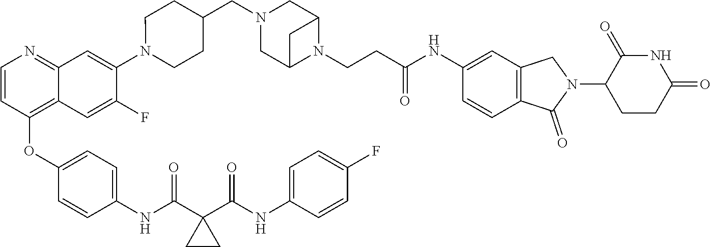 [SMILES: O=C1CCC(N2Cc3cc(NC(=O)CCN4C5CC4CN(CC4CCN(c6cc7nccc(Oc8ccc(NC(=O)C9(C(=O)Nc%10ccc(F)cc%10)CC9)cc8)c7cc6F)CC4)C5)ccc3C2=O)C(=O)N1]

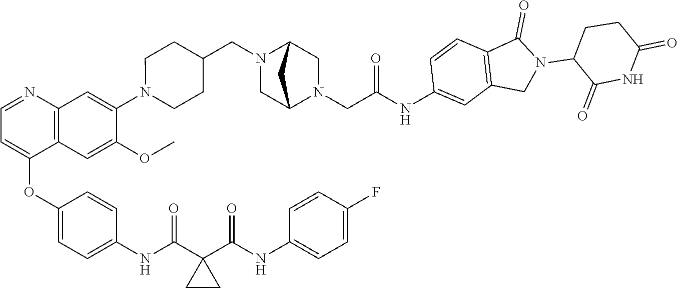 [SMILES: COc1cc2c(Oc3ccc(NC(=O)C4(C(=O)Nc5ccc(F)cc5)CC4)cc3)ccnc2cc1N1CCC(CN2C[C@@H]3C[C@H]2CN3CC(=O)Nc2ccc3c(c2)CN(C2CCC(=O)NC2=O)C3=O)CC1]

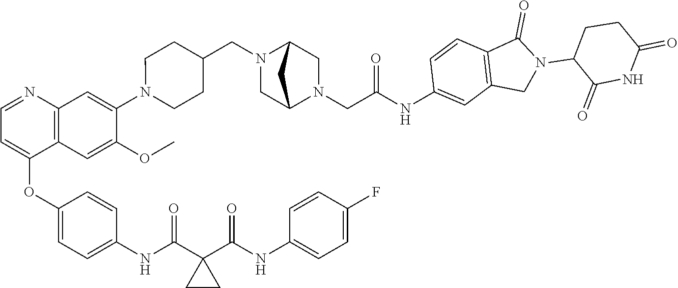 [SMILES: COc1cc2c(Oc3ccc(NC(=O)C4(C(=O)Nc5ccc(F)cc5)CC4)cc3)ccnc2cc1N1CCC(CN2C[C@@H]3C[C@H]2CN3CC(=O)Nc2ccc3c(c2)CN(C2CCC(=O)NC2=O)C3=O)CC1]

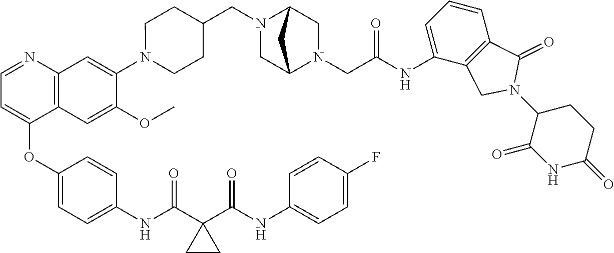 [SMILES: COc1cc2c(Oc3ccc(NC(=O)C4(C(=O)Nc5ccc(F)cc5)CC4)cc3)ccnc2cc1N1CCC(CN2C[C@@H]3C[C@H]2CN3CC(=O)Nc2cccc3c2CN(C2CCC(=O)NC2=O)C3=O)CC1]

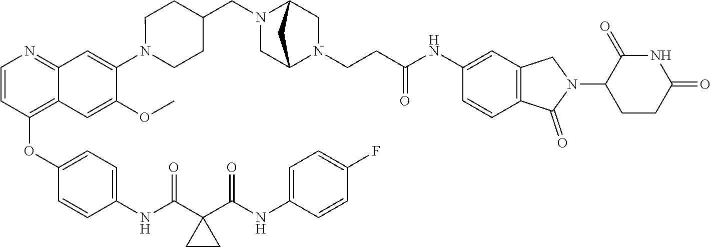 [SMILES: COc1cc2c(Oc3ccc(NC(=O)C4(C(=O)Nc5ccc(F)cc5)CC4)cc3)ccnc2cc1N1CCC(CN2C[C@@H]3C[C@H]2CN3CCC(=O)Nc2ccc3c(c2)CN(C2CCC(=O)NC2=O)C3=O)CC1]

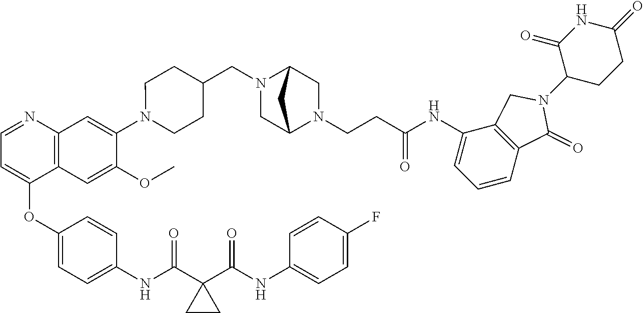 [SMILES: COc1cc2c(Oc3ccc(NC(=O)C4(C(=O)Nc5ccc(F)cc5)CC4)cc3)ccnc2cc1N1CCC(CN2C[C@@H]3C[C@H]2CN3CCC(=O)Nc2cccc3c2CN(C2CCC(=O)NC2=O)C3=O)CC1]

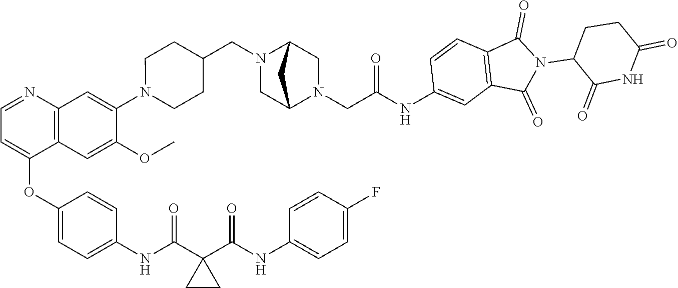 [SMILES: COc1cc2c(Oc3ccc(NC(=O)C4(C(=O)Nc5ccc(F)cc5)CC4)cc3)ccnc2cc1N1CCC(CN2C[C@@H]3C[C@H]2CN3CC(=O)Nc2ccc3c(c2)C(=O)N(C2CCC(=O)NC2=O)C3=O)CC1]

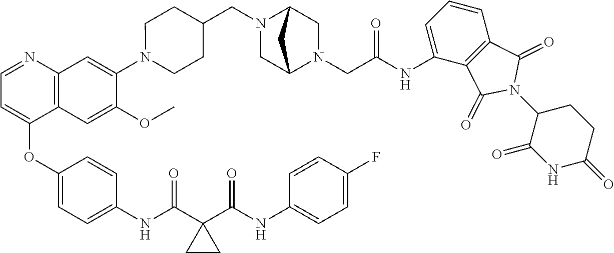 [SMILES: COc1cc2c(Oc3ccc(NC(=O)C4(C(=O)Nc5ccc(F)cc5)CC4)cc3)ccnc2cc1N1CCC(CN2C[C@@H]3C[C@H]2CN3CC(=O)Nc2cccc3c2C(=O)N(C2CCC(=O)NC2=O)C3=O)CC1]

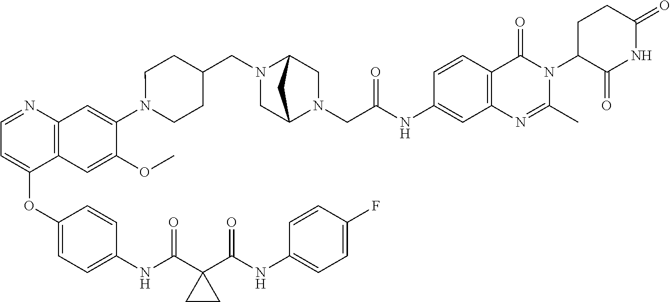 [SMILES: COc1cc2c(Oc3ccc(NC(=O)C4(C(=O)Nc5ccc(F)cc5)CC4)cc3)ccnc2cc1N1CCC(CN2C[C@@H]3C[C@H]2CN3CC(=O)Nc2ccc3c(=O)n(C4CCC(=O)NC4=O)c(C)nc3c2)CC1]

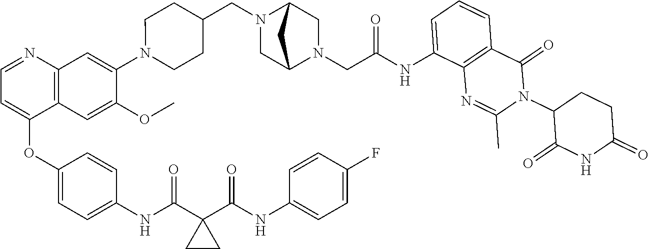 [SMILES: COc1cc2c(Oc3ccc(NC(=O)C4(C(=O)Nc5ccc(F)cc5)CC4)cc3)ccnc2cc1N1CCC(CN2C[C@@H]3C[C@H]2CN3CC(=O)Nc2cccc3c(=O)n(C4CCC(=O)NC4=O)c(C)nc23)CC1]

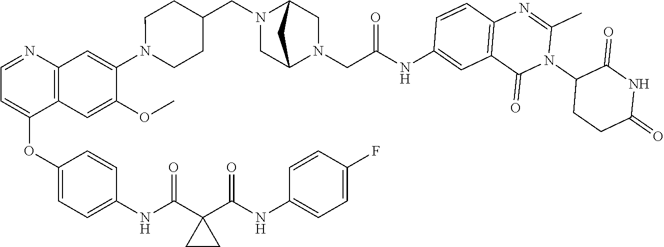 [SMILES: COc1cc2c(Oc3ccc(NC(=O)C4(C(=O)Nc5ccc(F)cc5)CC4)cc3)ccnc2cc1N1CCC(CN2C[C@@H]3C[C@H]2CN3CC(=O)Nc2ccc3nc(C)n(C4CCC(=O)NC4=O)c(=O)c3c2)CC1]

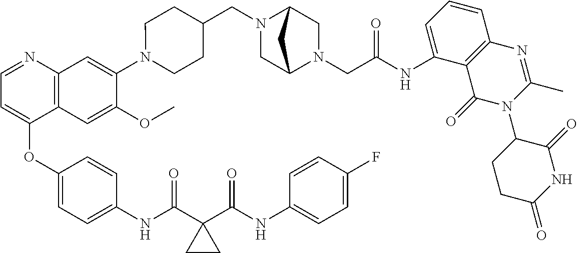 [SMILES: COc1cc2c(Oc3ccc(NC(=O)C4(C(=O)Nc5ccc(F)cc5)CC4)cc3)ccnc2cc1N1CCC(CN2C[C@@H]3C[C@H]2CN3CC(=O)Nc2cccc3nc(C)n(C4CCC(=O)NC4=O)c(=O)c23)CC1]

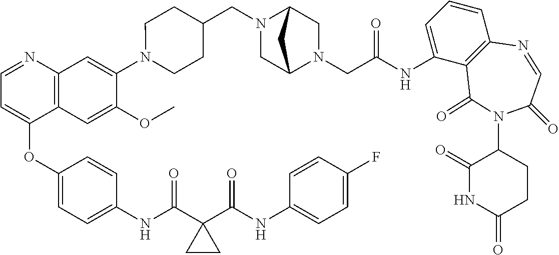 [SMILES: COc1cc2c(Oc3ccc(NC(=O)C4(C(=O)Nc5ccc(F)cc5)CC4)cc3)ccnc2cc1N1CCC(CN2C[C@@H]3C[C@H]2CN3CC(=O)Nc2cccc3ncc(=O)n(C4CCC(=O)NC4=O)c(=O)c23)CC1]

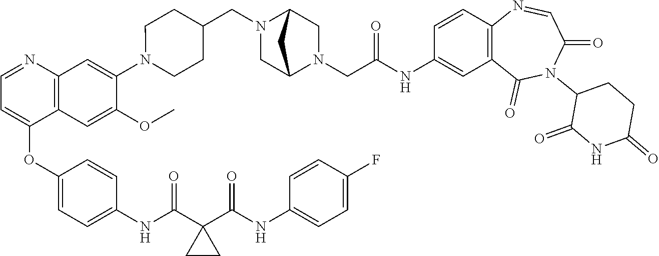 [SMILES: COc1cc2c(Oc3ccc(NC(=O)C4(C(=O)Nc5ccc(F)cc5)CC4)cc3)ccnc2cc1N1CCC(CN2C[C@@H]3C[C@H]2CN3CC(=O)Nc2ccc3ncc(=O)n(C4CCC(=O)NC4=O)c(=O)c3c2)CC1]

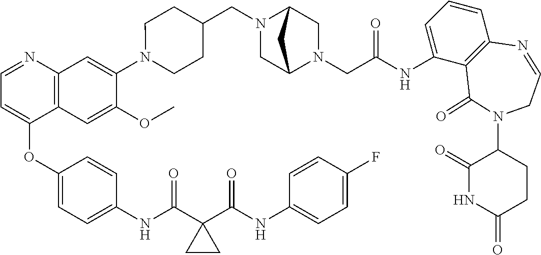 [SMILES: COc1cc2c(Oc3ccc(NC(=O)C4(C(=O)Nc5ccc(F)cc5)CC4)cc3)ccnc2cc1N1CCC(CN2C[C@@H]3C[C@H]2CN3CC(=O)Nc2cccc3c2C(=O)N(C2CCC(=O)NC2=O)CC=N3)CC1]

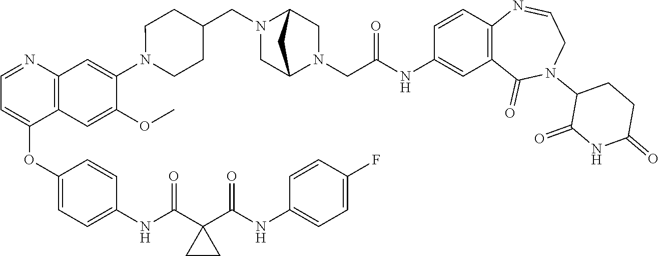 [SMILES: COc1cc2c(Oc3ccc(NC(=O)C4(C(=O)Nc5ccc(F)cc5)CC4)cc3)ccnc2cc1N1CCC(CN2C[C@@H]3C[C@H]2CN3CC(=O)Nc2ccc3c(c2)C(=O)N(C2CCC(=O)NC2=O)CC=N3)CC1]

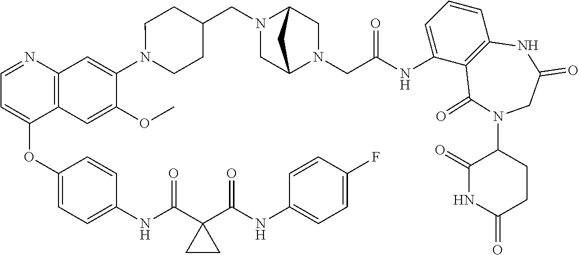 [SMILES: COc1cc2c(Oc3ccc(NC(=O)C4(C(=O)Nc5ccc(F)cc5)CC4)cc3)ccnc2cc1N1CCC(CN2C[C@@H]3C[C@H]2CN3CC(=O)Nc2cccc3c2C(=O)N(C2CCC(=O)NC2=O)CC(=O)N3)CC1]

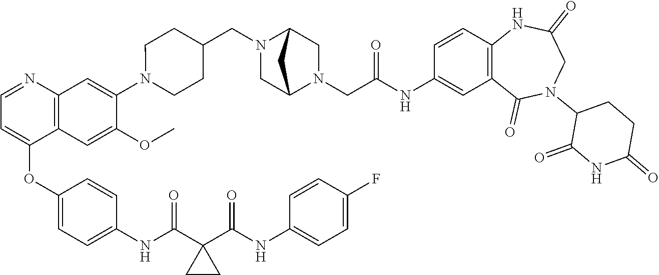 [SMILES: COc1cc2c(Oc3ccc(NC(=O)C4(C(=O)Nc5ccc(F)cc5)CC4)cc3)ccnc2cc1N1CCC(CN2C[C@@H]3C[C@H]2CN3CC(=O)Nc2ccc3c(c2)C(=O)N(C2CCC(=O)NC2=O)CC(=O)N3)CC1]

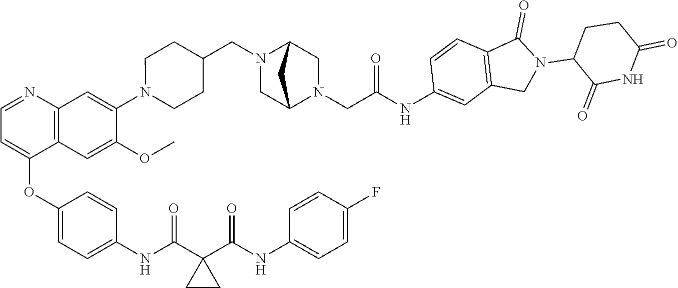 [SMILES: COc1cc2c(Oc3ccc(NC(=O)C4(C(=O)Nc5ccc(F)cc5)CC4)cc3)ccnc2cc1N1CCC(CN2C[C@@H]3C[C@H]2CN3CC(=O)Nc2ccc3c(c2)CN(C2CCC(=O)NC2=O)C3=O)CC1]

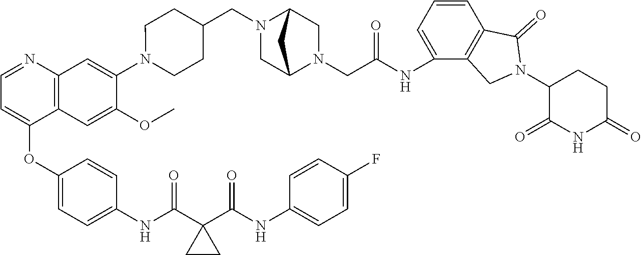 [SMILES: COc1cc2c(Oc3ccc(NC(=O)C4(C(=O)Nc5ccc(F)cc5)CC4)cc3)ccnc2cc1N1CCC(CN2C[C@@H]3C[C@H]2CN3CC(=O)Nc2cccc3c2CN(C2CCC(=O)NC2=O)C3=O)CC1]

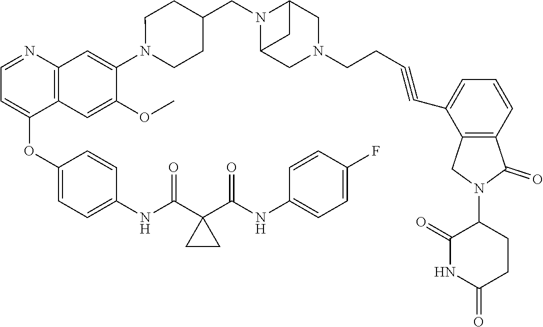 [SMILES: COc1cc2c(Oc3ccc(NC(=O)C4(C(=O)Nc5ccc(F)cc5)CC4)cc3)ccnc2cc1N1CCC(CN2C3CC2CN(CCC#Cc2cccc4c2CN(C2CCC(=O)NC2=O)C4=O)C3)CC1]

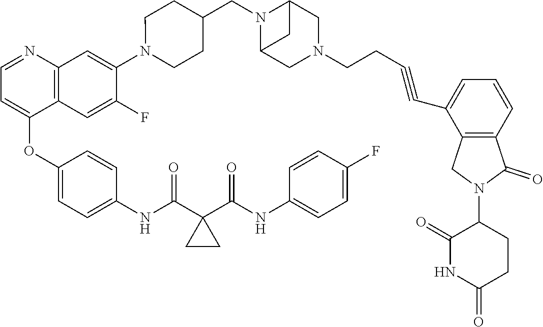 [SMILES: O=C1CCC(N2Cc3c(C#CCCN4CC5CC(C4)N5CC4CCN(c5cc6nccc(Oc7ccc(NC(=O)C8(C(=O)Nc9ccc(F)cc9)CC8)cc7)c6cc5F)CC4)cccc3C2=O)C(=O)N1]

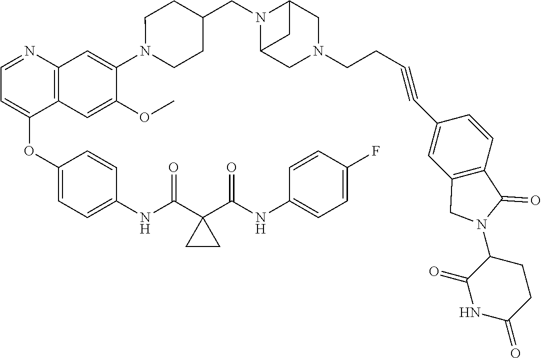 [SMILES: COc1cc2c(Oc3ccc(NC(=O)C4(C(=O)Nc5ccc(F)cc5)CC4)cc3)ccnc2cc1N1CCC(CN2C3CC2CN(CCC#Cc2ccc4c(c2)CN(C2CCC(=O)NC2=O)C4=O)C3)CC1]

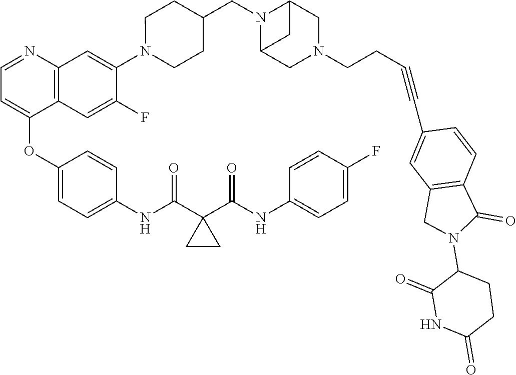 [SMILES: O=C1CCC(N2Cc3cc(C#CCCN4CC5CC(C4)N5CC4CCN(c5cc6nccc(Oc7ccc(NC(=O)C8(C(=O)Nc9ccc(F)cc9)CC8)cc7)c6cc5F)CC4)ccc3C2=O)C(=O)N1]

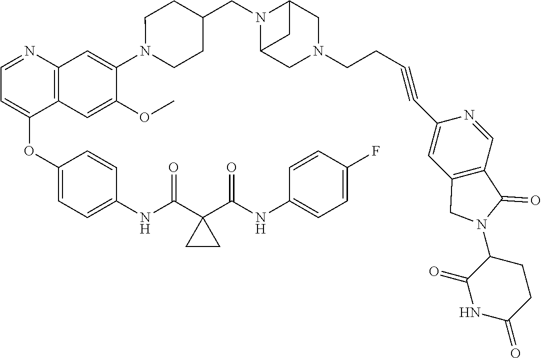 [SMILES: COc1cc2c(Oc3ccc(NC(=O)C4(C(=O)Nc5ccc(F)cc5)CC4)cc3)ccnc2cc1N1CCC(CN2C3CC2CN(CCC#Cc2cc4c(cn2)C(=O)N(C2CCC(=O)NC2=O)C4)C3)CC1]

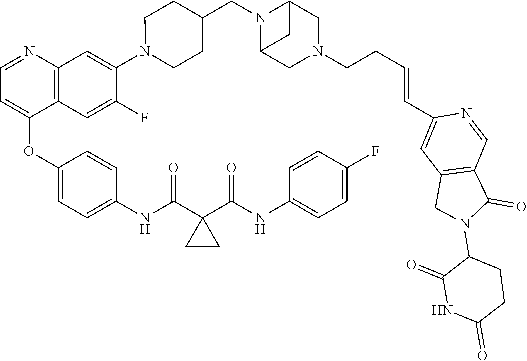 [SMILES: O=C1CCC(N2Cc3cc(/C=C/CCN4CC5CC(C4)N5CC4CCN(c5cc6nccc(Oc7ccc(NC(=O)C8(C(=O)Nc9ccc(F)cc9)CC8)cc7)c6cc5F)CC4)ncc3C2=O)C(=O)N1]

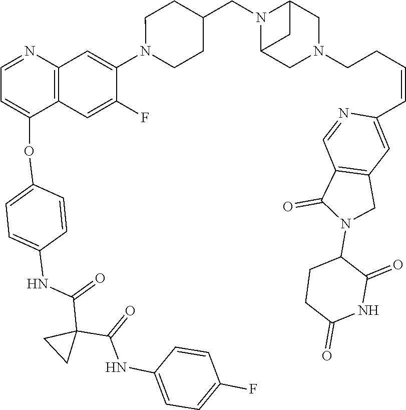 [SMILES: O=C1CCC(N2Cc3cc(/C=C\CCN4CC5CC(C4)N5CC4CCN(c5cc6nccc(Oc7ccc(NC(=O)C8(C(=O)Nc9ccc(F)cc9)CC8)cc7)c6cc5F)CC4)ncc3C2=O)C(=O)N1]

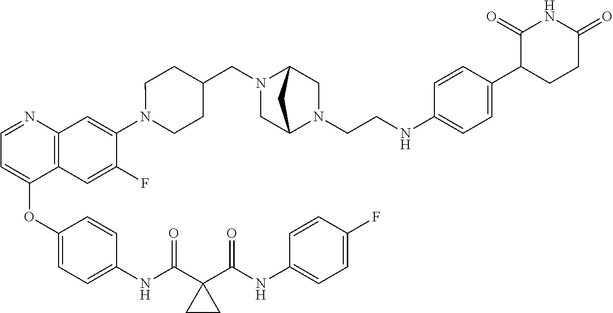 [SMILES: O=C1CCC(c2ccc(NCCN3C[C@@H]4C[C@H]3CN4CC3CCN(c4cc5nccc(Oc6ccc(NC(=O)C7(C(=O)Nc8ccc(F)cc8)CC7)cc6)c5cc4F)CC3)cc2)C(=O)N1]

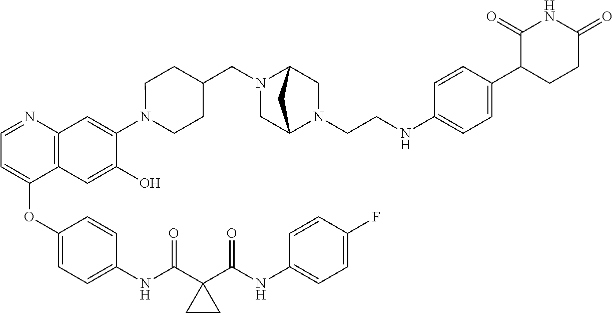 [SMILES: O=C1CCC(c2ccc(NCCN3C[C@@H]4C[C@H]3CN4CC3CCN(c4cc5nccc(Oc6ccc(NC(=O)C7(C(=O)Nc8ccc(F)cc8)CC7)cc6)c5cc4O)CC3)cc2)C(=O)N1]

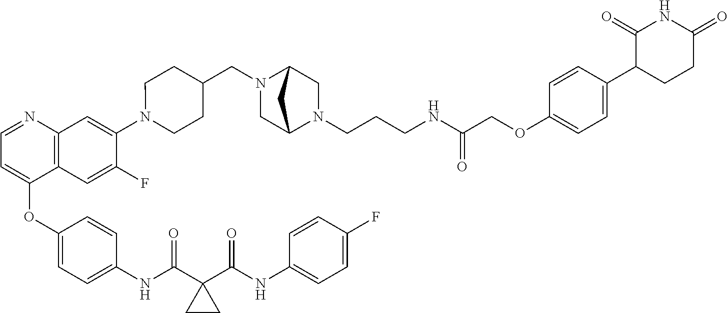 [SMILES: O=C(COc1ccc(C2CCC(=O)NC2=O)cc1)NCCCN1C[C@@H]2C[C@H]1CN2CC1CCN(c2cc3nccc(Oc4ccc(NC(=O)C5(C(=O)Nc6ccc(F)cc6)CC5)cc4)c3cc2F)CC1]

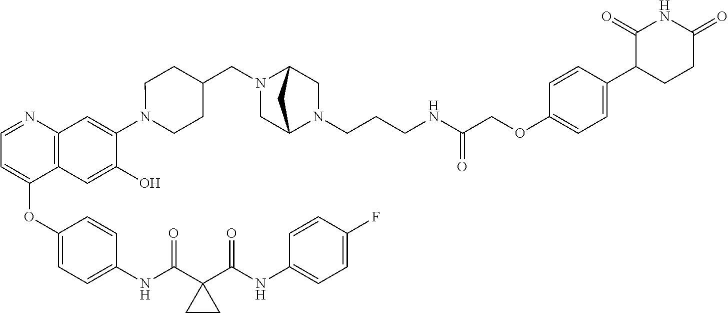 [SMILES: O=C(COc1ccc(C2CCC(=O)NC2=O)cc1)NCCCN1C[C@@H]2C[C@H]1CN2CC1CCN(c2cc3nccc(Oc4ccc(NC(=O)C5(C(=O)Nc6ccc(F)cc6)CC5)cc4)c3cc2O)CC1]

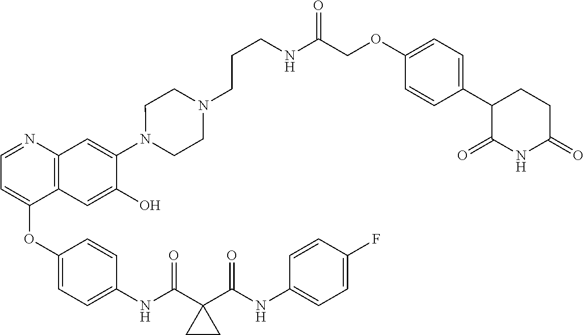 [SMILES: O=C(COc1ccc(C2CCC(=O)NC2=O)cc1)NCCCN1CCN(c2cc3nccc(Oc4ccc(NC(=O)C5(C(=O)Nc6ccc(F)cc6)CC5)cc4)c3cc2O)CC1]

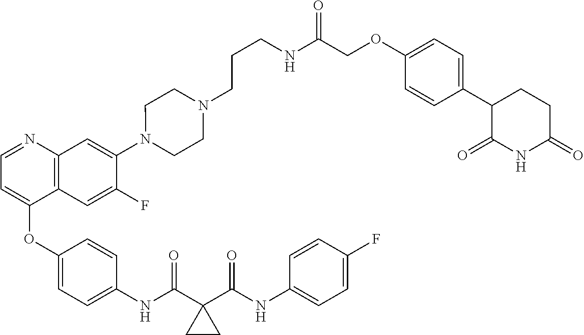 [SMILES: O=C(COc1ccc(C2CCC(=O)NC2=O)cc1)NCCCN1CCN(c2cc3nccc(Oc4ccc(NC(=O)C5(C(=O)Nc6ccc(F)cc6)CC5)cc4)c3cc2F)CC1]

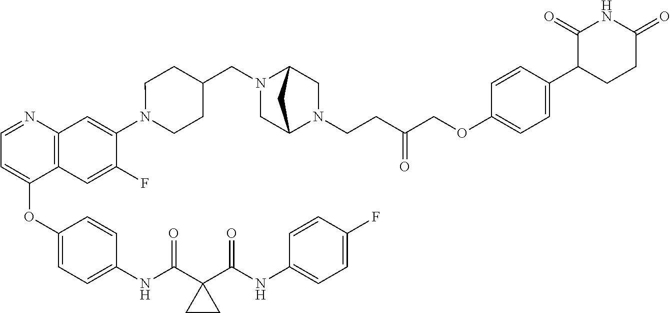 [SMILES: O=C(CCN1C[C@@H]2C[C@H]1CN2CC1CCN(c2cc3nccc(Oc4ccc(NC(=O)C5(C(=O)Nc6ccc(F)cc6)CC5)cc4)c3cc2F)CC1)COc1ccc(C2CCC(=O)NC2=O)cc1]

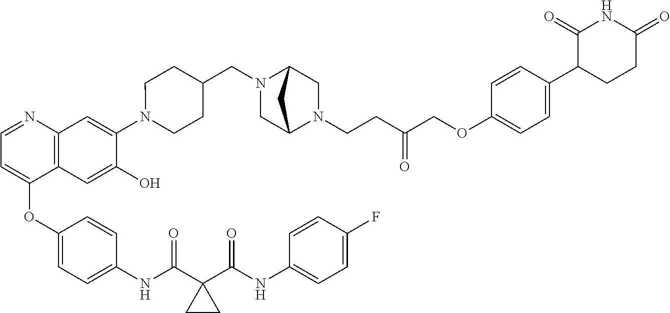 [SMILES: O=C(CCN1C[C@@H]2C[C@H]1CN2CC1CCN(c2cc3nccc(Oc4ccc(NC(=O)C5(C(=O)Nc6ccc(F)cc6)CC5)cc4)c3cc2O)CC1)COc1ccc(C2CCC(=O)NC2=O)cc1]

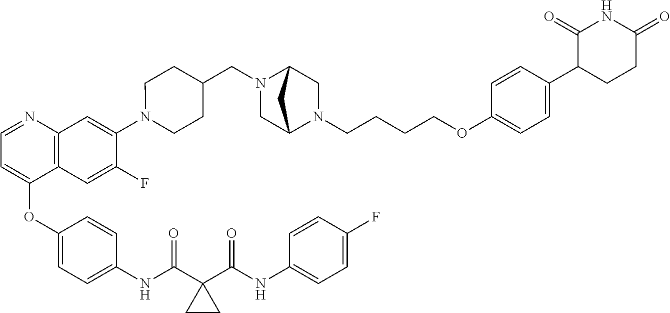 [SMILES: O=C1CCC(c2ccc(OCCCCN3C[C@@H]4C[C@H]3CN4CC3CCN(c4cc5nccc(Oc6ccc(NC(=O)C7(C(=O)Nc8ccc(F)cc8)CC7)cc6)c5cc4F)CC3)cc2)C(=O)N1]

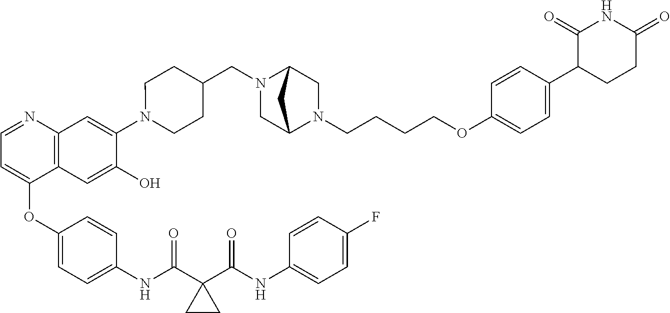 [SMILES: O=C1CCC(c2ccc(OCCCCN3C[C@@H]4C[C@H]3CN4CC3CCN(c4cc5nccc(Oc6ccc(NC(=O)C7(C(=O)Nc8ccc(F)cc8)CC7)cc6)c5cc4O)CC3)cc2)C(=O)N1]

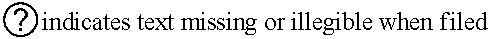

### DESCRIPTION OF THE DRAWINGS

In order to facilitate understanding of the content of the present invention, further detailed explanations will be provided below based on specific embodiments of the present invention and in conjunction with the accompanying drawings,

FIG. 1 the inhibitory effects of different compounds of the present invention on MKN45 tumor cells;

FIG. 2: the inhibitory effects of different compounds of the present invention on SNU5 tumor cells;

FIG. 3: the immunoblotting experiment of the degradation activity of DDR1 in tumor cell MKN45 by different compounds of the present invention;

FIG. 4: the measurement of the degradation activity of different compounds of the present invention on DDR1 in tumor cell MKN45;

FIG. 5: the inhibitory curve of TPD12140, a representative compound of the present invention, on subcutaneous tumor growth and weight change in mice;

FIG. 6: the results of the degradation mechanism of target protein DDR1 by the representative compound TPD12140 of the present invention.

### DETAILED DESCRIPTION OF THE INVENTION

The following further illustrates the present invention in conjunction with embodiments and their accompanying drawings. These embodiments are only for more detailed and specific instructions and should not be understood as limiting the present invention in any form. The present invention can be implemented in various ways limited and covered by the claims.

Although many materials and operating methods used in the present invention are well-known in the art, the present invention is still described in as much detail as possible. In the following text, unless otherwise specified, the materials and operating methods used are well-known in the art. Unless otherwise defined, all terms used in the present invention (including technical and scientific terms) have the same meaning as those commonly understood by those skilled in the art to which the present invention belongs. The synthesis of the target compound of the present invention adopts the method shown in the reaction flowchart. The structure and purity of the product were confirmed by nuclear magnetic resonance, mass spectrometry, and liquid chromatography. Unless otherwise specified, the starting materials used in the synthesis method for preparing the compounds of the present invention are commercially available or can be prepared according to known methods in the art or described herein.

General synthesis of compounds: In view of the present disclosure or through the explanatory methods shown in the following general scheme, prepare the disclosed compounds using methods known to those skilled in the art. If necessary, suitable protective groups can be used in the synthesis in either of the general schemes. It should be understood that the described implementation plans and examples are not intended to limit the scope of this disclosure, and the claims presented herein are intended to cover all implementation plans and examples, whether or not explicitly presented herein.

### Example 1: Synthesis of Compound TPD005305

This embodiment was used to prepare the synthetic compound TPD005305 by the following synthetic route:

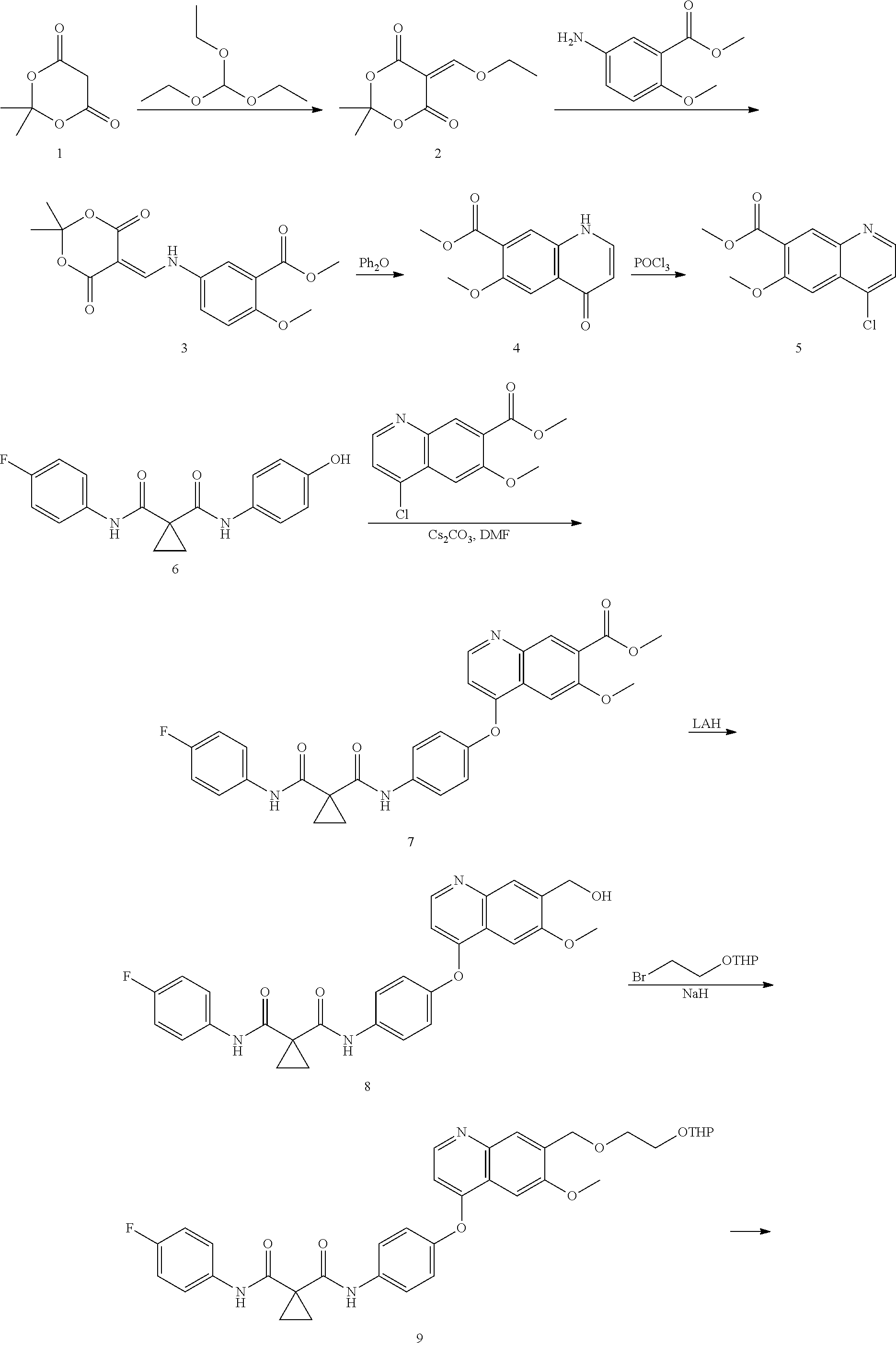

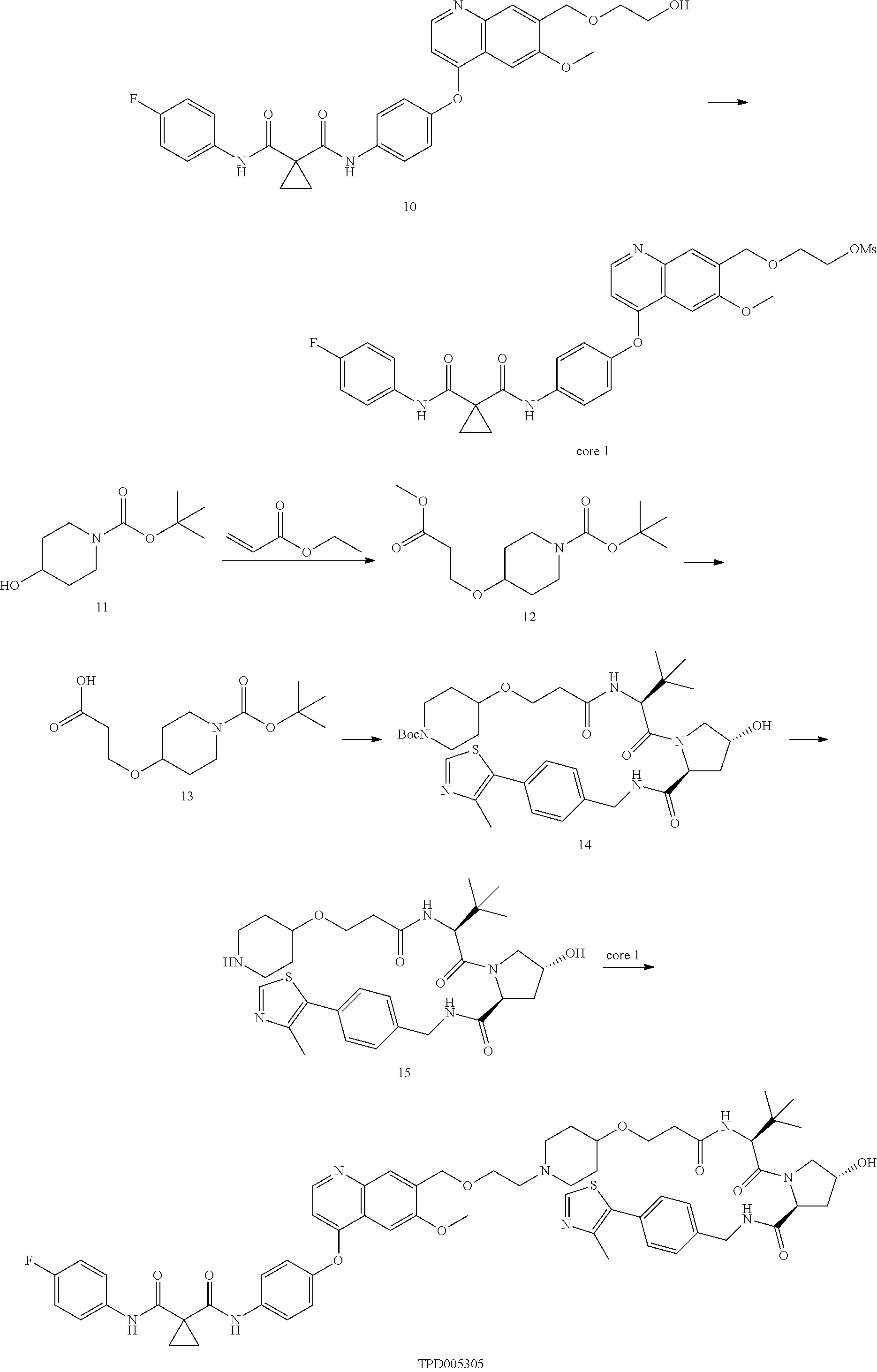

Compound 1 (20 g, 139 mol) was added to a 250 mL single-necked vial and triethyl orthoformate (70 mL) was heated to 100° C. and stirred for 2 hours. The reaction solution was reduced to room temperature, filtered by adding methyl tert-butyl ether, the filter cake was drenched with methyl tert-butyl ether and dried to give compound 2 (23 g, yellow solid), calculated yield: 82.7%.

Compound 2 (5 g, 25 mol), methyl 5-amino-2-methoxybenzoate (4.5 g, 25 mmol) and isopropanol (50 mL) were added to a 100 mL single-necked vial and stirred for 3 hours at room temperature. The reaction solution was filtered and the filter cake was eluted with methyl tert-butyl ether and dried to give compound 3 (8 g, yellow solid), calculated yield: 95%.

Diphenyl ether (80 mL) was added to a 100 mL single-necked flask and heated up to 240° C. compound 3 (8 g, 23.9 mmol) was added in one portion and stirred for 30 min, the reaction solution was reduced to room temperature, filtered by addition of Methyl tert-butyl ether, and the filter cake was drenched with Methyl tert-butyl ether and dried to give compound 4 (4.5 g, brown solid) in crude form.

In a 100 mL single-necked flask was added phosphorus oxychloride (50 mL), compound 4 (4.5 g, crude), warmed to 100° C. and stirred for 2 hours, the reaction solution will be to room temperature, concentrated to remove phosphorous trichloride. Add methyl tert-butyl ether filtration, the filter cake was washed with methyl tert-butyl ether, dried to obtain compound 5 (1 g, brown solid), calculated yield: two steps 16.7%.

In a 100 mL single-necked vial, compound 6 (2 g, 6.4 mmol), compound 5 (1.6 g, 6.4 mmol), cesium carbonate (4.2 g, 12.7 mmol), and N,N-dimethylformamide (20 mL) were added and warmed to 100° C. with stirring overnight. The reaction solution was reduced to room temperature, poured into water, extracted by adding ethyl acetate (30 mL×3), and the organic phase was concentrated by washing with water and drying. The crude product was purified by column chromatography to give compound 7 (1 g, yellow solid), calculated yield: 29.8%.

In a 100 mL three-necked flask, lithium aluminum hydride (144 mg, 3.8 mmol), anhydrous tetrahydrofuran (10 mL), and a tetrahydrofuran solution (5 mL) of compound 7 (1 g, 1.9 mmol) was added dropwise at 0° C. under nitrogen protection. After dropwise addition, the reaction solution was stirred at room temperature for 1 h. The reaction solution was quenched with sodium sulfate decahydrate and filtered, the filter cake was washed with dichloromethane:methanol (20:1), and the filtrate was concentrated to obtain compound 8 (500 mg, yellow solid), calculated yield: 52.6%.

To a 50 mL three-necked vial was added compound 8 (500 mg, 1 mmol), N,N-dimethylformamide (10 mL) and sodium hydride (200 mg, 5 mmol) under nitrogen protection. Stirred at room temperature for 30 min and compound 2-(2-bromoethoxy)tetrahydro-2H-pyran (250 mg, 1.2 mmol) was added and stirred overnight at room temperature. The reaction solution was quenched by pouring into ammonium chloride solution, extracted with dichloromethane (20 mL×3) and the organic phase was dried and concentrated to give compound 9 (230 mg, yellow solid), calculated yield: 36.6%.

Compound 9 (1.2 g, 1.9 mmol), methanol (10 mL), 2N hydrochloric acid (10 mL) were added to a 50 mL single-necked vial and stirred at room temperature for 2 hours. The reaction solution was poured into water and sodium carbonate solution was adjusted pH=7-8, the filter cake was dried to give compound 10 (700 mg, yellow solid), calculated yield: 90%.

Compound 10 (400 mg, 0.73 mmol), triethylamine (148 mg, 1.5 mmol), dichloromethane (10 mL) were added to a 100 mL triple-necked vial, methylsulfonyl chloride (126 mg, 1.1 mmol) was added dropwise at 0° C., and stirred for 2 hr at room temperature after addition. The reaction solution was poured into water, extracted with ethyl acetate (10 mL×3), and the organic phase was dried and concentrated to obtain compound core 1 (400 mg, yellow liquid) as crude.

A tetrahydrofuran solution (150 mL) of compound 11 (15 g, 74.6 mmol), ethyl acrylate (8.96 g, 89.6 mol) and potassium tert-butanolate (12.5 g, 112 mmol) was stirred overnight at room temperature, the reaction solution was quenched with 1N HCl (100 mL) and extracted with ethyl acetate (50 mL×2). The combined organic phases were washed with water (50 mL) and dried over a column with anhydrous sodium sulfate to give compound 12 (6.5 g, 30%).

To a methanolic solution of compound 12 (6.5 g, 22.6 mmol) (60 mL) was added sodium hydroxide (1.8 g, 45.3 mmol) and stirred at room temperature overnight. The reaction solution was spun dry, diluted with water, adjusted pH=6 with 1N hydrochloric acid, extracted with ethyl acetate (30 mL×3), and the combined organic phases were dried over anhydrous sodium sulfate and spun dry to give compound 13 (2 g, 32%).

In a 100 mL single-necked vial, compound 13 (150 mg, 0.55 mmol), (2S,4R)-1-((S)-2-amino-3,3-dimethylbutanoyl)-4-hydroxy-N-(4-(4-methylthiazol-5-yl)benzyl)pyrrolidine-2-carboxamide hydrochloride (257 mg, 0.55 mmol, 1-hydroxybenzotriazole (111.4 mg, 0.82 mmol), 1-(3-dimethylaminopropyl)-3-ethylcarbodiimide hydrochloride (158 mg, 0.82 mmol), N,N-diisopropylethylamine (248 mg, 1.9 mmol), and dichloromethane (5 mL) were stirred overnight at room temperature. The reaction solution was washed and dried and concentrated, and the crude product was purified by preparative plate to give compound 14 (230 mg, yellow solid), calculated yield: 61.2%.

In a 100 mL single-necked vial, compound 14 (230 mg, 0.34 mmol), hydrochloric acid/ethyl acetate (2N, 5 mL), was added and stirred at room temperature for 30 min. The reaction solution was concentrated to give compound 15 (200 mg, yellow solid), calculated yield: 96%.

Compound 15 (200 mg, 0.32 mmol), core 1 (200 mg, 0.32 mmol), sodium iodide (97 mg, 0.64 mmol), potassium carbonate (89 mg, 0.64 mmol), N,N-dimethylformamide (10 mL) were added to a 100 mL single-necked vial and stirred for 3 hours at 80° C. The reaction solution was reduced to room temperature, poured into water and extracted with ethyl acetate (10 ml×3). The organic phase was washed with water and dried and concentrated, and the crude was purified by high pressure preparation to give the compound TPD005305 (50 mg, yellow solid), calculated yield: 14%. LCMS (ESI) m/z calcd. for C₆₀H₆₉FN₈O₁₀S [M+H]⁺ 1112.5; found 1112.7. The compound structure is confirmed.

### Example 2: Synthesis of Compound TPD005313

This embodiment was used to prepare the synthetic compound TPD005313 by the following synthetic route:

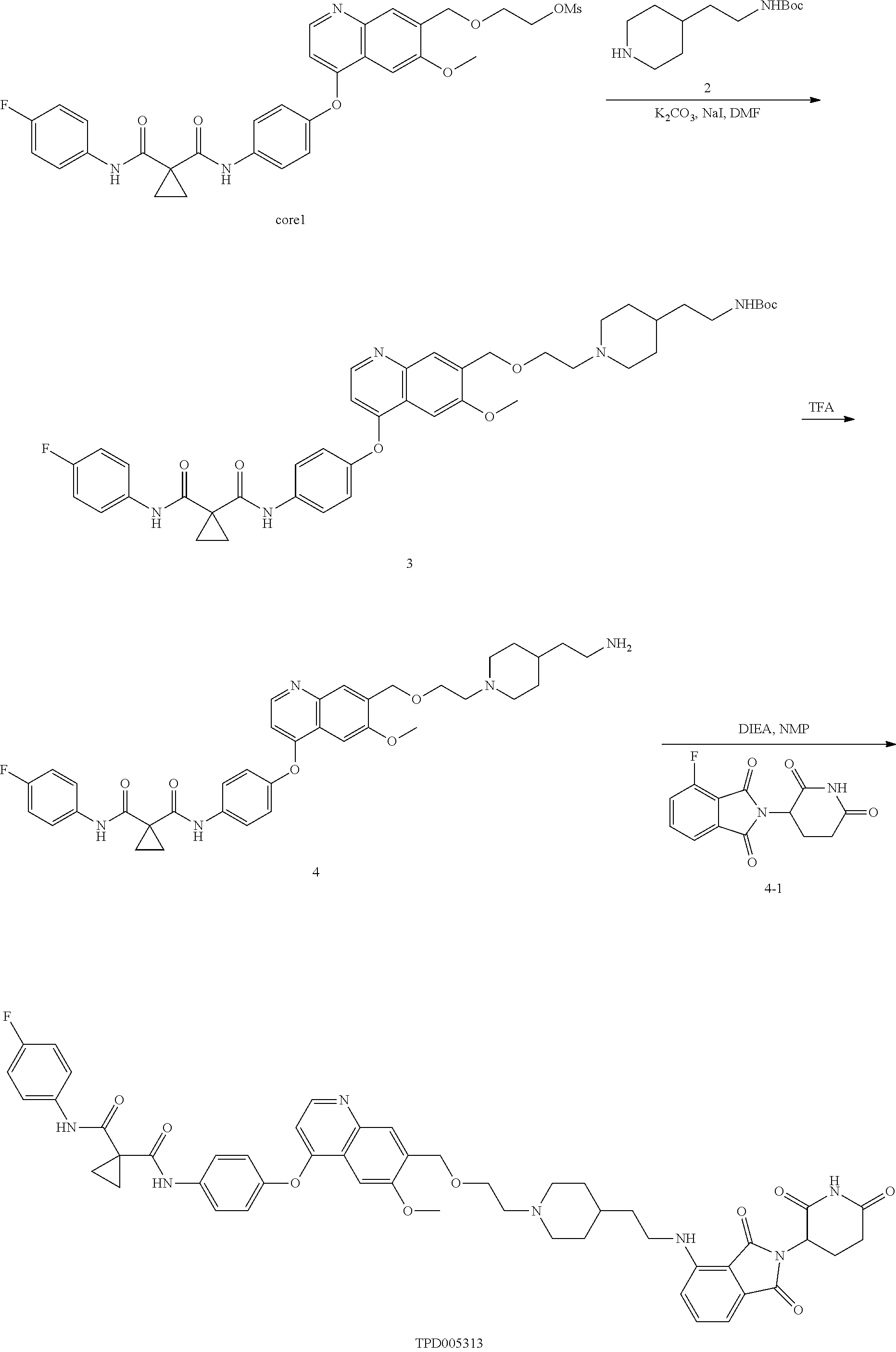

A solution of compound core 1 (200 mg, 0.32 mmol), compound 2 (109.8 mg, 0.48 mmol), sodium iodide (97 mg, 0.64 mmol) and potassium carbonate (88.6 mg, 0.67 mmol), synthesized according to the method steps in Example 1, in DMF (5 ml), was stirred at 100° C. overnight. After completion of the reaction the reaction solution was added to (20 ml) of ice water and extracted with EA (10 ml×2), the organic phase was dried over anhydrous sodium sulfate and concentrated to give the crude compound 3 as a yellow solid (200 mg, 82.6% yield).

Compound 3 (200 mg, 0.26 mmol) was dissolved in DCM (5 ml), TFA (5 ml) was added and the reaction was carried out at room temperature for 2 hours. Spin drying of the reaction solution gave compound 4 as a yellow solid (160 mg, 92.4% yield).

Compound 4 (80 mg, 0.12 mmol) and compound 4-1 (40.4 mg, 0.15 mmol) were dissolved in NMP (5 ml), DIEA (31 mg, 0.24 mmol) was added at room temperature and the reaction solution reacted at 100° C. overnight. The reaction solution was prepared under high pressure to give the compound TPD005313 as a yellow solid (4 mg, 3.6% yield). LCMS (ESI) m/z calcd. for C₅₀H₅₁FN₇O₉ [M+H]⁺ 912.4; found 912.5. The compound structure is confirmed.

### Example 3: Synthesis of Compound TPD005315

This embodiment was used to prepare the synthetic compound TPD005315 by the following synthetic route:

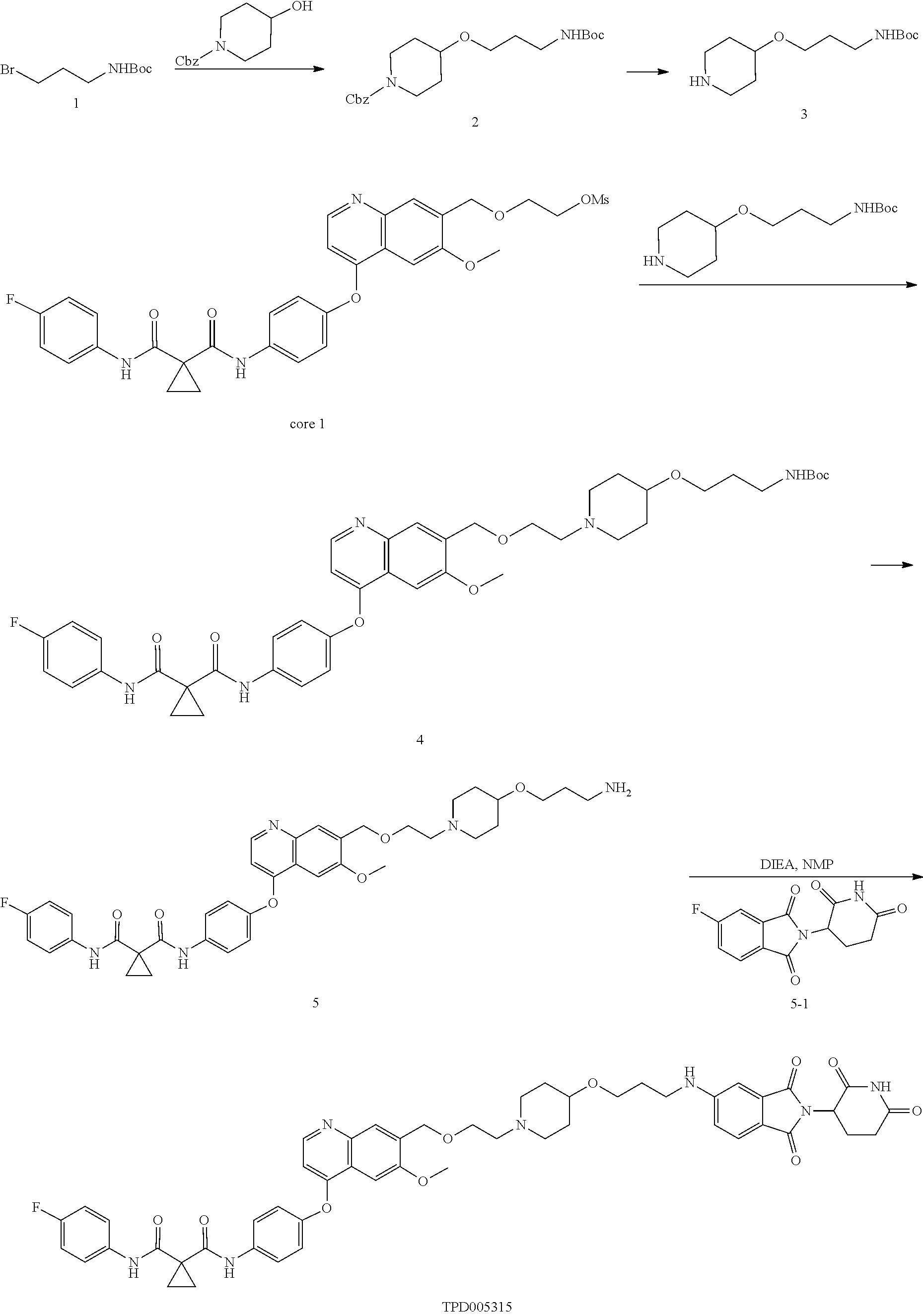

Compound 1 (10 g, 42.2 mmol), benzyl 4-hydroxypiperidine-1-carboxylate (10 g, 42.2 mmol) and potassium carbonate (12.5 g, 90.9 mmol) were dissolved in DMF (100 ml) and stirred at 50° C. overnight. After completion of the reaction the reaction solution was added to (200 ml) ice water and extracted with EA (100 ml×2), the organic layer was dried and concentrated with anhydrous sodium sulfate and purified over a column to give compound 2 as a yellow solid (6 g, 36.3% yield).

Compound 2 (6 g, 16.5 mmol) was dissolved in methanol, palladium carbon (1 g) was added, and 20 Psi hydrogen at room temperature overnight, filtered, and spun-dried to give compound 3 as a yellow solid (2.2 g, 55.7% yield).

Compound Corel (100 mg, 0.16 mmol) and compound 3 (62 mg, 0.24 mmol) were dissolved in DMF (2 ml), NaI (48 mg, 0.32 mmol) and potassium carbonate (44.3 mg, 0.32 mmol) were added, and the reaction was carried out at 100° C. overnight. The reaction was completed by pouring the reaction solution into water, EA (10 ml×2) was extracted, the organic phase was washed with saturated brine (10 mL×2), dried over anhydrous sodium sulfate, and the solvent was spun dry to give the crude compound 4 as a yellow solid (110 mg, 88% yield).

Compound 4 (110 mg, 0.14 mmol) was dissolved in EA (2 ml), 4 N HCl/EA (2 ml) was added and the reaction was carried out at room temperature for 2 hours. The reaction was completed by vacuum spin drying the solvent under high pressure to prepare a yellow solid of compound 5 (10 mg, 10% yield).

Compound 5 (10 mg, 0.015 mmol) and compound 5-1 (5 mg, 0.018 mmol) were dissolved in NMP (2 ml) and DIEA (4 mg, 0.03 mmol) was added at room temperature and the reaction solution reacted overnight at 100. The reaction solution was prepared under high pressure to give the compound TPD005315 as a yellow solid (1 mg, 7.3% yield). LCMS (ESI) m/z calcd. for C₅₁H₅₃FN₇O₁₀ [M+H]⁺ 942.4; found 942.5. The compound structure is confirmed.

### Example 4: Synthesis of Compound TPD005316

This embodiment was used to prepare the synthetic compound TPD005316 by the following

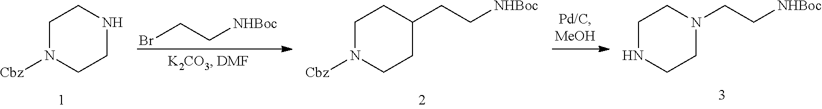 [SMILES: O=C(OCc1ccccc1)N1CCNCC1 ; CC(C)(C)OC(=O)NCCBr ; CC(C)(C)OC(=O)NCCC1CCN(C(=O)OCc2ccccc2)CC1 ; CC(C)(C)OC(=O)NCCN1CCNCC1]

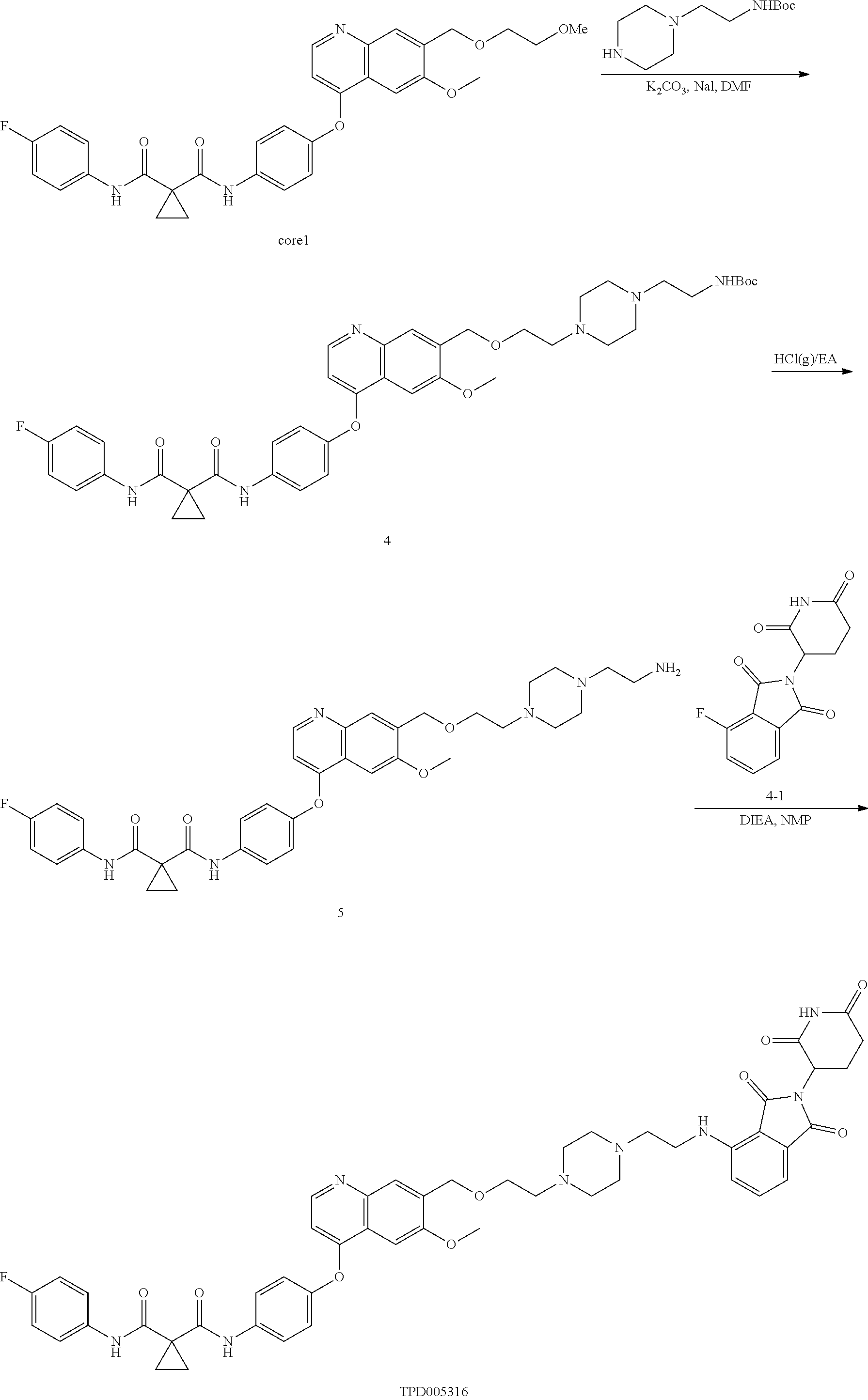 [SMILES: COCCOCc1cc2nccc(Oc3ccc(NC(=O)C4(C(=O)Nc5ccc(F)cc5)CC4)cc3)c2cc1OC ; CC(C)(C)OC(=O)NCCN1CCNCC1 ; COc1cc2c(Oc3ccc(NC(=O)C4(C(=O)Nc5ccc(F)cc5)CC4)cc3)ccnc2cc1COCCN1CCN(CCNC(=O)OC(C)(C)C)CC1 ; COc1cc2c(Oc3ccc(NC(=O)C4(C(=O)Nc5ccc(F)cc5)CC4)cc3)ccnc2cc1COCCN1CCN(CCN)CC1 ; O=C1CCC(N2C(=O)c3cccc(F)c3C2=O)C(=O)N1 ; COc1cc2c(Oc3ccc(NC(=O)C4(C(=O)Nc5ccc(F)cc5)CC4)cc3)ccnc2cc1COCCN1CCN(CCNc2cccc3c2C(=O)N(C2CCC(=O)NC2=O)C3=O)CC1]

Compound 1 (10 g, 45.4 mmol), tert-butyl (2-bromoethyl) carbamate (12.16 g, 54.5 mmol) and potassium carbonate (12.5 g, 90.9 mmol) were dissolved in DMF (100 ml) and stirred at 50° C. overnight. After completion of the reaction the reaction solution was added to ice water (200 ml) and extracted with EA (100 ml×2), the organic layer was dried and concentrated with anhydrous sodium sulfate and purified over a column to give compound 2 as a yellow solid (6 g, 36.3% yield).

Compound 2 (6 g, 16.5 mmol) was dissolved in methanol, palladium carbon (1 g) was added, and the mixture was left at room temperature overnight under 20 Psi hydrogen. Filtration and spin drying gave compound 3 as a yellow solid (2.2 g, 59.4% yield).

Compound Corel (100 mg, 0.16 mmol) and compound 3 (62 mg, 0.24 mmol) prepared as in Example 1 were dissolved in DMF (2 ml), NaI (48 mg, 0.32 mmol) and potassium carbonate (44.3 mg, 0.32 mmol) were added, and the reaction was carried out at 100° C. overnight. The reaction was completed the reaction solution was poured into water, EA (10 ml×2) was added to extract, the organic phase was washed with saturated brine (10 mL×2), dried over anhydrous sodium sulfate, and the solvent was spun dry to give the crude compound 4 as a yellow solid (102 mg, 84.2% yield).

Compound 4 (102 mg, 0.13 mmol) was dissolved in EA (2 ml), 4N HCl/EA (2 ml) was added and the reaction was carried out at room temperature for 2 hours. The reaction was completed vacuum spin drying the solvent as a yellow solid of compound 5 (80 mg, 90% yield).

Compound 5 (80 mg, 0.12 mmol) and compound 4-1 (40.4 mg, 0.14 mmol) were dissolved in NMP (2 ml) and DIEA (31 mg, 0.24 mmol) was added at room temperature and the reaction solution reacted overnight at 100. The reaction solution was prepared under high pressure to give the compound TPD005316 as a yellow solid (2 mg, 1.8% yield). LCMS (ESI) m/z calcd. for C₄₉H₅₀FN₈O₉ [M+H]⁺ 913.4; found 913.3. The compound structure is confirmed.

### Example 5: Synthesis of Compound TPD005317

This embodiment was used to prepare the synthetic compound TPD005317 by the following synthetic route:

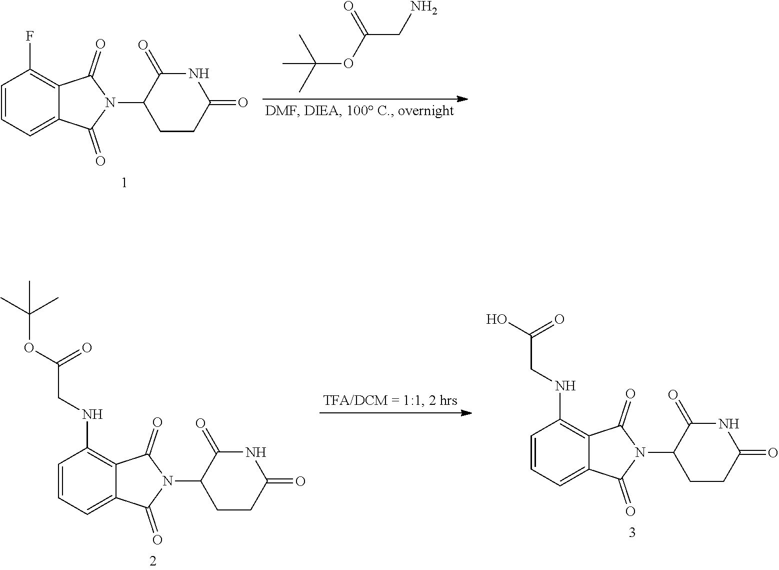

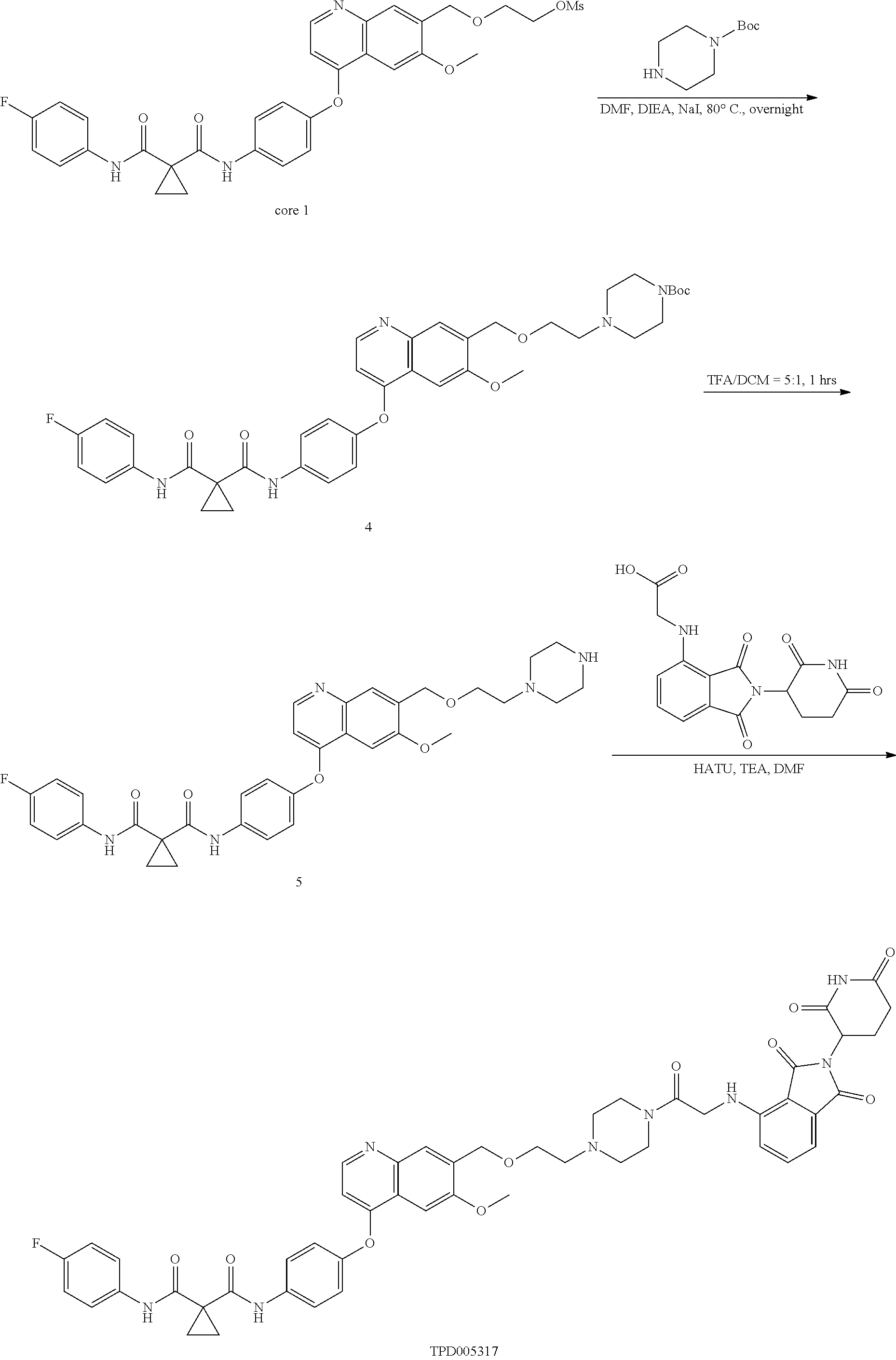 [SMILES: COc1cc2c(Oc3ccc(NC(=O)C4(C(=O)Nc5ccc(F)cc5)CC4)cc3)ccnc2cc1COCCOS(C)(=O)=O ; CC(C)(C)OC(=O)N1CCNCC1 ; COc1cc2c(Oc3ccc(NC(=O)C4(C(=O)Nc5ccc(F)cc5)CC4)cc3)ccnc2cc1COCCN1CCN(C(=O)OC(C)(C)C)CC1 ; COc1cc2c(Oc3ccc(NC(=O)C4(C(=O)Nc5ccc(F)cc5)CC4)cc3)ccnc2cc1COCCN1CCNCC1 ; O=C(O)CNc1cccc2c1C(=O)N(C1CCC(=O)NC1=O)C2=O ; COc1cc2c(Oc3ccc(NC(=O)C4(C(=O)Nc5ccc(F)cc5)CC4)cc3)ccnc2cc1COCCN1CCN(C(=O)CNc2cccc3c2C(=O)N(C2CCC(=O)NC2=O)C3=O)CC1]

Compound 1 (450 mg, 1.63 mmol), tert-butyl glycinate (256.3 mg, 1.96 mmol) and DIEA (420.6 mg, 3.26 mmol) in DMF (10 ml) were stirred at 100° C. overnight. After completion of the reaction the reaction solution was added to ice water (30 ml) and extracted with EA (20 ml×2), the organic layer was dried with Na₂ SO₄ and concentrated to give a yellow solid crude compound 2 (610 mg, 101.3% yield).

Compound 2 (300 mg, 0.78 mmol) was dissolved in DCM (5 ml), TFA (5 ml) was added, and the reaction was carried out at room temperature for 2 h. The reaction solution was spun dry to obtain a yellow solid of compound 3 (280 mg, crude).

The aforementioned compounds Corel (100 mg, 0.16 mmol) and N-BOC-piperazine (59.7 mg, 0.32 mmol) were dissolved in DMF (5 ml), NaI (48 mg, 0.32 mmol) and potassium carbonate (44.3 mg, 0.32 mmol) were added, and the reaction was carried out at 80° C. overnight. The reaction was completed by pouring the reaction solution into water, EA (10 ml×2) was extracted, the organic phase was washed with saturated saline (10 mL×2), dried with anhydrous sodium sulfate, spun-dried with solvent DCM:MeOH=10:1, and purified by preparative plate to give compound 4 as a yellow solid (54 mg, 47.3% yield).

Compound 4 (54 mg, 0.75 mmol) was dissolved in (2 ml) DCM, TFA (0.4 ml) was added and the reaction was carried out at room temperature for 1 hour. The reaction was completed by vacuum spin drying the solvent to give compound 5 as a yellow solid (80 mg, 90% yield).

Compound 5 (80 mg, 0.13 mmol) and compound 3 (66 mg, 0.2 mmol) were dissolved in DMF (2 ml), HATU (76 mg, 0.2 mmol) and TEA (26 mg, 0.26 mmol) were added at room temperature and the reaction solution reacted at 100° C. overnight. The reaction solution was prepared under high pressure to give the compound TPD005317 as a yellow solid (17 mg, 14.1% yield). LCMS (ESI) m/z calcd. for C₄₉H₄₈FN₈O₁₀ [M+H]⁺ 927.3; found 927.2. The compound structure is confirmed.

### Example 6: Synthesis of Compound TPD005401

This embodiment was used to prepare the synthetic compound TPD005401 by the following synthetic route:

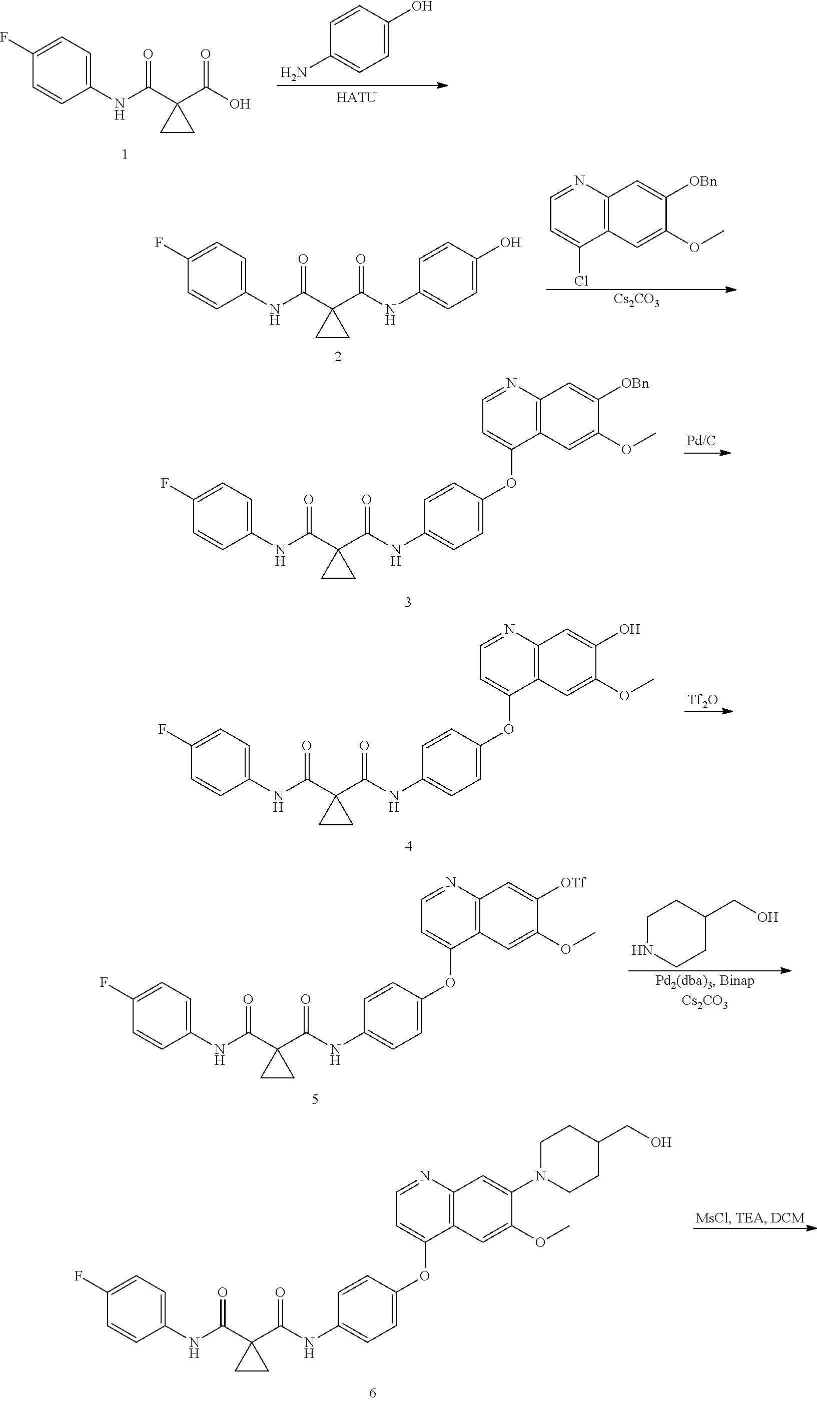

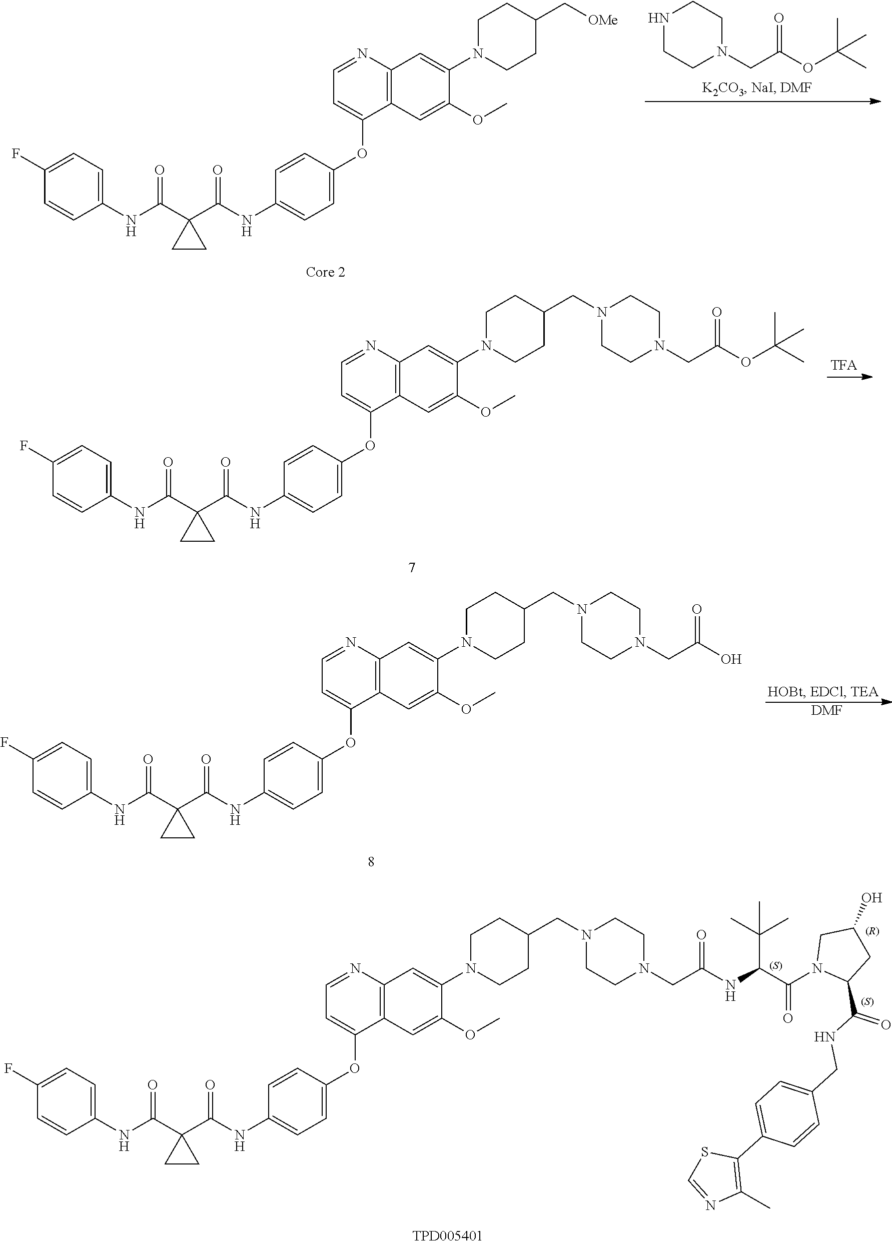

To a 250 ml triple-necked vial was added compound 1 (10.0 g, 0.05 mol), 4-aminophenol (7.33 g, 0.07 mol) and N,N-dimethylformamide (100 ml). After dissolution, N,N,N′,N′-tetramethyl-O-(7-azabenzotriazol-1-yl)urea hexafluorophosphate (25.5 g, 0.07 mol) and N,N-diisopropylethylamine (17.3 g, 0.13 mol) were added and stirred at room temperature overnight. Quenched with water, extracted with ethyl acetate 3 times, the combined organic phases were washed with water 3 times, saturated brine once, dried over sodium sulfate and spun dry. The crude product was pulped with dichloromethane, filtered and the filter cake was dried to obtain compound 2 (9.00 g, white solid), yield: 64.3%.

A 250 ml single-necked vial was charged with compound 2 (10.0 g, 0.03 mol), 7-benzyloxy-4-chloro-6-methoxyquinoline 5-amino-2-methoxybenzoate methyl ester (8.70 g, 0.03 mol), cesium carbonate (21.0 g, 0.06 mol), and dimethylsulfoxide (100 ml), and the reaction solution was stirred overnight at 120° C., quenched by adding water. It was extracted with ethyl acetate 3 times, washed with water once, washed with saturated brine once, dried with anhydrous sodium sulfate and spun dry. The crude product was passed through a column with petroleum ether/ethyl acetate (1:1) and dichloromethane/methanol (20:1-10:1) to obtain compound 3 (10 g, brown solid), yield: 48.3%.

A 250 ml hydrogenation flask was charged with compound 3 (8.00 g, 0.013 mol), palladium carbon (3.00 g) and tetrahydrofuran (40 ml) and methanol (40 ml). The air was replaced three times with hydrogen and stirred overnight at 40° C. in a 20 Psi hydrogen atmosphere. The reaction solution was filtered through diatomaceous earth and the filtrate was spin dried to give compound 4 (6.00 g, yellow solid), yield: 88.9%.

A 250 ml three-necked vial was charged with compound 4 (7.00 g, 0.01 mol), pyridine (14.0 g, 0.18 mol), dichloromethane (35 ml), and tetrahydrofuran (35 ml). Trifluoromethanesulfonic anhydride (26.0 g, 0.09 mol) was added dropwise at 0° C. with stirring at room temperature overnight. Quenched with water, extracted with dichloromethane 3 times, the combined organic phases were washed once with saturated brine, dried with anhydrous sodium sulfate, spun dry and purified over a column (petroleum ether/ethyl acetate=30:1-3:1) to give compound 5 (4.00 g, yellow solid), yield: 44.9%.

A 20 ml reaction vial was charged with compound 5 (100 mg, 0.16 mmol), 4-hydroxymethylpiperidine (40.0 mg, 0.35 mmol), tris(dibenzylideneacetone)dipalladium (30.0 mg, 0.03 mmol), 1,1′-binaphthyl-2,2′-bisdiphenyphosphine (40.0 mg, 0.06 mmol), cesium carbonate (105 mg. 0.32 mmol) and 1,4-dioxane (10 ml), nitrogen displacement and stirring at 100° C. overnight. Quenched with water, extracted with ethyl acetate 3 times, the combined organic phases were washed once with saturated brine, dried over anhydrous sodium sulfate and spun dry. The crude product was purified by column purification (petroleum ether/ethyl acetate=30:1-2:1) to obtain compound 6 (30.0 mg, yellow solid), yield: 31.8%.

A 100 ml triple vial was charged with compound 6 (380 mg, 0.64 mmol), triethylamine (99.0 mg, 0.97 mmol), dichloromethane (5 ml) and methanesulfonyl chloride (89.0 mg, 0.78 mmol). The reaction solution was stirred at 25° C. overnight, quenched with water, extracted 3 times with dichloromethane, the combined organic phases were washed once with water, dried over anhydrous sodium sulfate and spun dry to give compound core2 (320 mg, yellow solid), yield: 74.3%.

A 100 ml single-necked vial was charged with compound core2 (200 mg, 0.30 mmol), tert-butyl 2-(piperidin-1-yl)acetate (723 mg, 0.36 mmol), potassium carbonate (83.0 mg, 0.60 mmol), sodium iodide (90.0 mg, 0.60 mmol), N,N-dimethylformamide (20 ml). The reaction solution was stirred at 100° C. overnight. Quenched with water, extracted with ethyl acetate 3 times, the combined organic phases were washed with water 3 times, saturated brine once, dried over anhydrous sodium sulfate and spun-dried to give compound 7 (200 mg, yellow solid), yield: 86.5%.

To a 100 ml single mouth flask was added compound 7 (400 mg, 0.52 mmol), dichloromethane (10 ml) and trifluoroacetic acid (2 ml), the reaction solution was stirred at room temperature overnight, spun dry and pulped with methyl tert-butyl ether to give the crude compound 8 (400 mg, yellow solid).

A 100 ml triple-necked vial was charged with compound 8 (500 mg, 0.70 mmol), (2S,4R)-1-((S)-2-amino-3,3-dimethylbutanoyl)-4-hydroxy-N-(4-(4-methylthiazol-5-yl)benzyl)pyrrolidine-2-carboxamide hydrochloride (492 mg, 1.05 mmol), and triethylamine (213 mg. 2.11 mmol), dissolved with DCM (5 ml), 1-hydroxybenzotriazole (114 mg, 0.25 mmol) and 1-ethyl-(3-dimethylaminopropyl)carbodiimide hydrochloride (161 mg, 0.85 mmol) were added and stirred at room temperature overnight. Quenched with water, extracted with dichloromethane 3 times, the combined organic phases were washed with water 3 times, saturated brine once, dried over anhydrous sodium sulfate and spun dry. The crude product was purified by high pressure preparation to give compound TPD005401 (35 mg, yellow solid), yield: 4.7%. LCMS (ESI) m/z calcd. for C₆₁H₇₂FN₁₀O₈S [M+H]⁺ 1122.5; found 1122.6. The compound structure is confirmed.

### Example 7: Synthesis of Compound TPD005402B

This embodiment was used to prepare the synthetic compound TPD005402B by the following synthetic route:

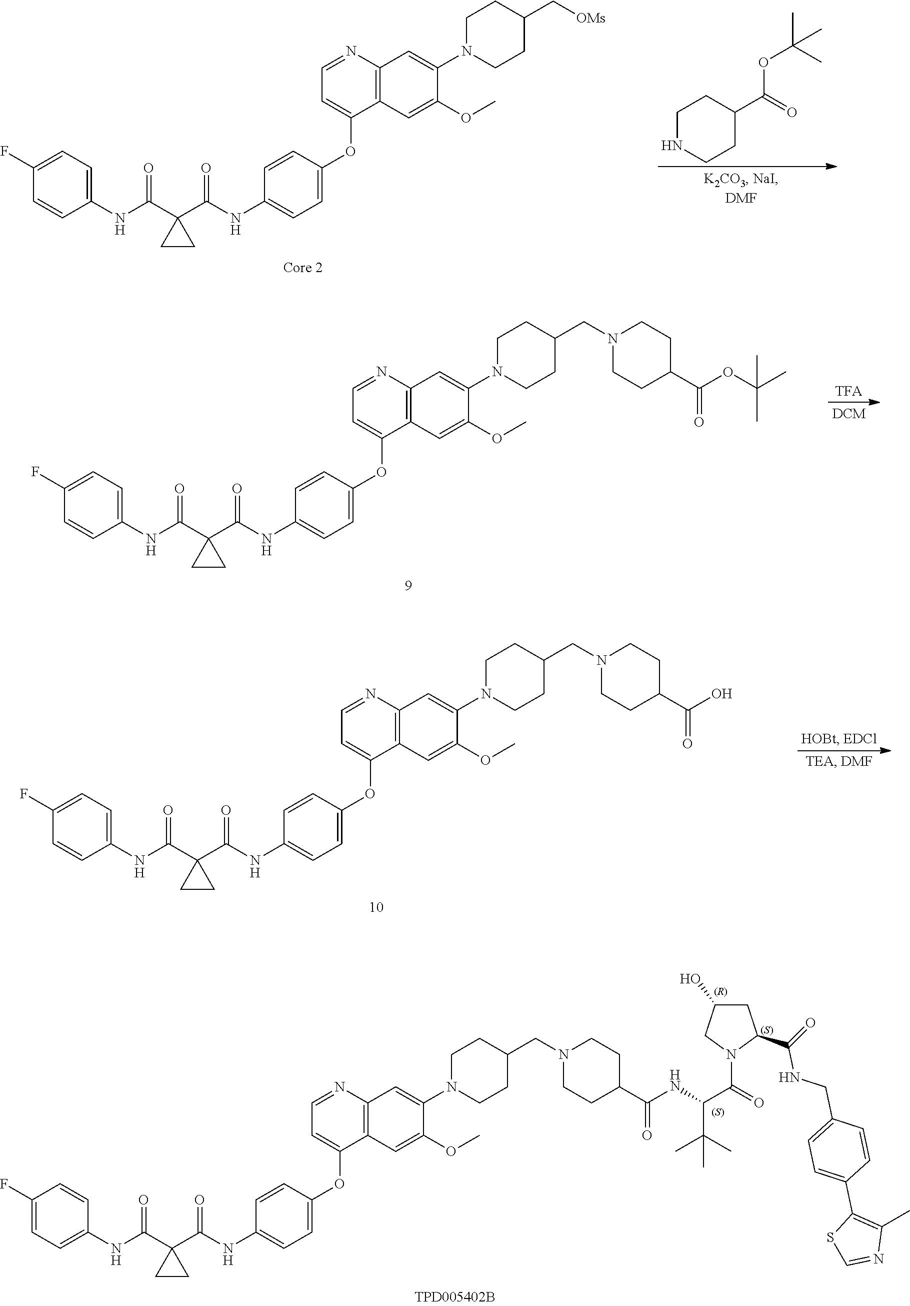

A 100 ml single-necked vial was charged with compound core2 (200 mg, 0.30 mmol) prepared according to the steps in Example 6, tert-butyl piperidine-4-carboxylate (67.0 mg, 0.36 mmol), potassium carbonate (83.4 mg, 0.60 mmol), sodium iodide (90.0 mg, 0.60 mmol), and N,N-dimethylformamide (20 ml). The reaction solution was stirred at 80° C. overnight. Quenched with water, extracted with ethyl acetate 3 times, the combined organic phases were washed with water 3 times, saturated brine once, dried over anhydrous sodium sulfate and spun-dried to give compound 9 (100 mg, yellow solid), yield: 44.0%.

In a 100 ml single-necked vial, compound 9 (100 mg, 0.13 mmol), dichloromethane (10 ml) and trifluoroacetic acid (2 ml) were added, the reaction solution was stirred at room temperature overnight, spun dry, and pulped with methyl tert-butyl ether to obtain the crude compound 10 (80 mg, yellow solid), yield: 92.3%.

A 100 ml triple-necked vial was charged with compound 10 (95 mg, 0.15 mmol), (2S,4R)-1-((S)-2-amino-3,3-dimethylbutanoyl)-4-hydroxy-N-(4-(4-methylthiazol-5-yl)benzyl)pyrrolidine-2-carboxamide hydrochloride (102 mg, 0.22 mmol) and triethylamine (44.2 mg. 0.44 mmol), dissolved in dichloromethane (10 ml), 1-hydroxybenzotriazole (23.6 mg, 0.18 mmol), 1-ethyl-(3-dimethylaminopropyl)carbodiimide hydrochloride (33.4 mg, 0.18 mmol) were added and stirred at room temperature overnight. Quenched with water, extracted with dichloromethane 3 times, the combined organic phases were washed with water 3 times, saturated brine once, dried with anhydrous sodium sulfate and spun dry. The crude product was purified by high pressure preparation to give compound TPD00542B (19 mg, yellow solid), yield: 11.7%. LCMS (ESI) m/z calcd. for C₆₁H₇₁FN₉O₈S [M+H]⁺ 1107.5; found 1107.5. The compound structure is confirmed.

### Example 8: Synthesis of Compound TPD005406

This embodiment was used to prepare the synthetic compound TPD005406 by the following synthetic route:

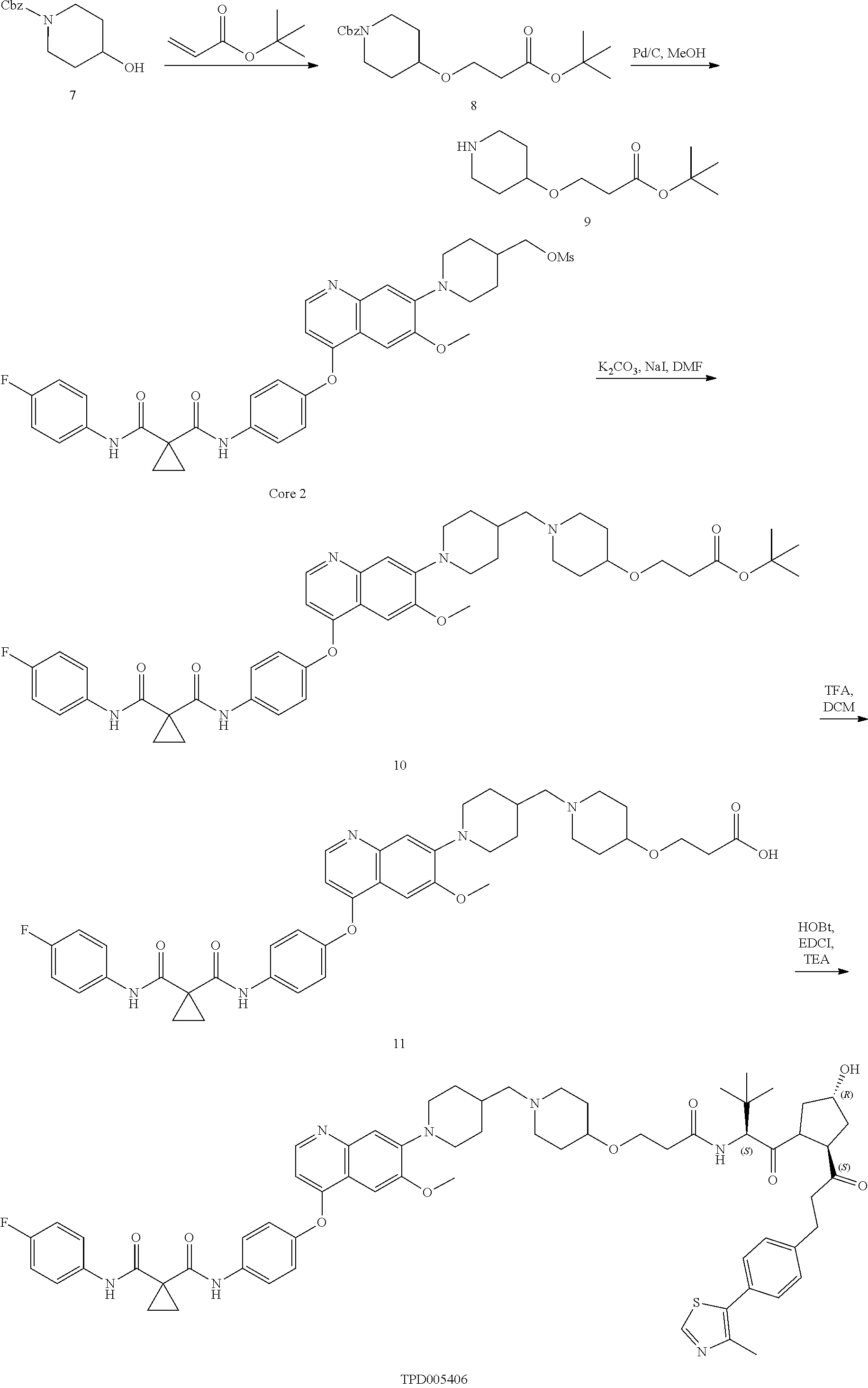

A tetrahydrofuran solution (150 ml) of compound 7 (10 g, 42.6 mmol), tert-butyl acrylate (8.17 g. 63.8 mol) and potassium tert-butanolate (12.5 g, 112 mmol) was taken and stirred at room temperature overnight, the reaction solution was quenched with 1N HCl (100 ml) and extracted with ethyl acetate (50 ml×2). The combined organic phases were washed with water (50 ml) and dried over a column with anhydrous sodium sulfate to give compound 8 (5 g, 32.5%).

To a methanolic solution (50 ml) of compound 8 (5 g, 13.8 mmol) was added palladium carbon (1 g) and stirred overnight at room temperature in a hydrogen atmosphere (30 psi). The reaction solution was filtered and spun dry to give compound 9 (1.5 g, 47.6%).

A 100 ml single-necked vial was charged with compound core 2 (100 mg, 0.15 mmol), tert-butyl 3-(piperidin-4-acyloxy)propionate (68.7 mg, 0.30 mmol), potassium carbonate (83.0 mg, 0.60 mmol), sodium iodide (90.0 mg, 0.60 mmol) and N,N-dimethylformamide (10 ml). The reaction solution was stirred at 100° C. overnight. Quenched with water, extracted with ethyl acetate 3 times, the combined organic phases were washed with water 3 times, saturated brine once, dried over anhydrous sodium sulfate and spun-dried to give compound 10 (80 mg, yellow solid), yield: 66.7%.

To a 100 ml single necked vial was added compound 7 (80 mg, 0.11 mmol), dichloromethane (10 ml) and trifluoroacetic acid (2 ml), the reaction solution was stirred at room temperature overnight, spun dry and pulped with methyl tert-butyl ether to give the crude compound 11 (50 mg, yellow solid).

A 100 ml triple-necked vial was charged with compound 11 (50 mg, 0.07 mmol), (2S,4R)-1-((S)-2-amino-3,3-dimethylbutanoyl)-4-hydroxy-N-(4-(4-methylthiazol-5-yl)benzyl)pyrrolidine-2-carboxamide hydrochloride (49 mg, 0.11 mmol) and triethylamine (21.3 mg. 0.2 mmol), dissolved with DCM (2 ml), 1-hydroxybenzotriazole (11.4 mg, 0.03 mmol) and 1-ethyl-(3-dimethylaminopropyl)carbodiimide hydrochloride (16.1 mg, 0.09 mmol) were added and stirred at room temperature overnight. Quenched with water, extracted with dichloromethane 3 times, the combined organic phases were washed with water 3 times, saturated brine once, dried over anhydrous sodium sulfate and spun dry. The crude product was purified by high pressure preparation to give compound TPD005406 (10 mg, yellow solid), yield: 12.8%. LCMS (ESI) m/z calcd. for C₆₃H₇₅FN₉O₉S [M+H]⁺ 1151.5; found 1151.3. The compound structure is confirmed.

### Example 9: Synthesis of Compound TPD005407

This embodiment was used to prepare the synthetic compound TPD005407 by the following

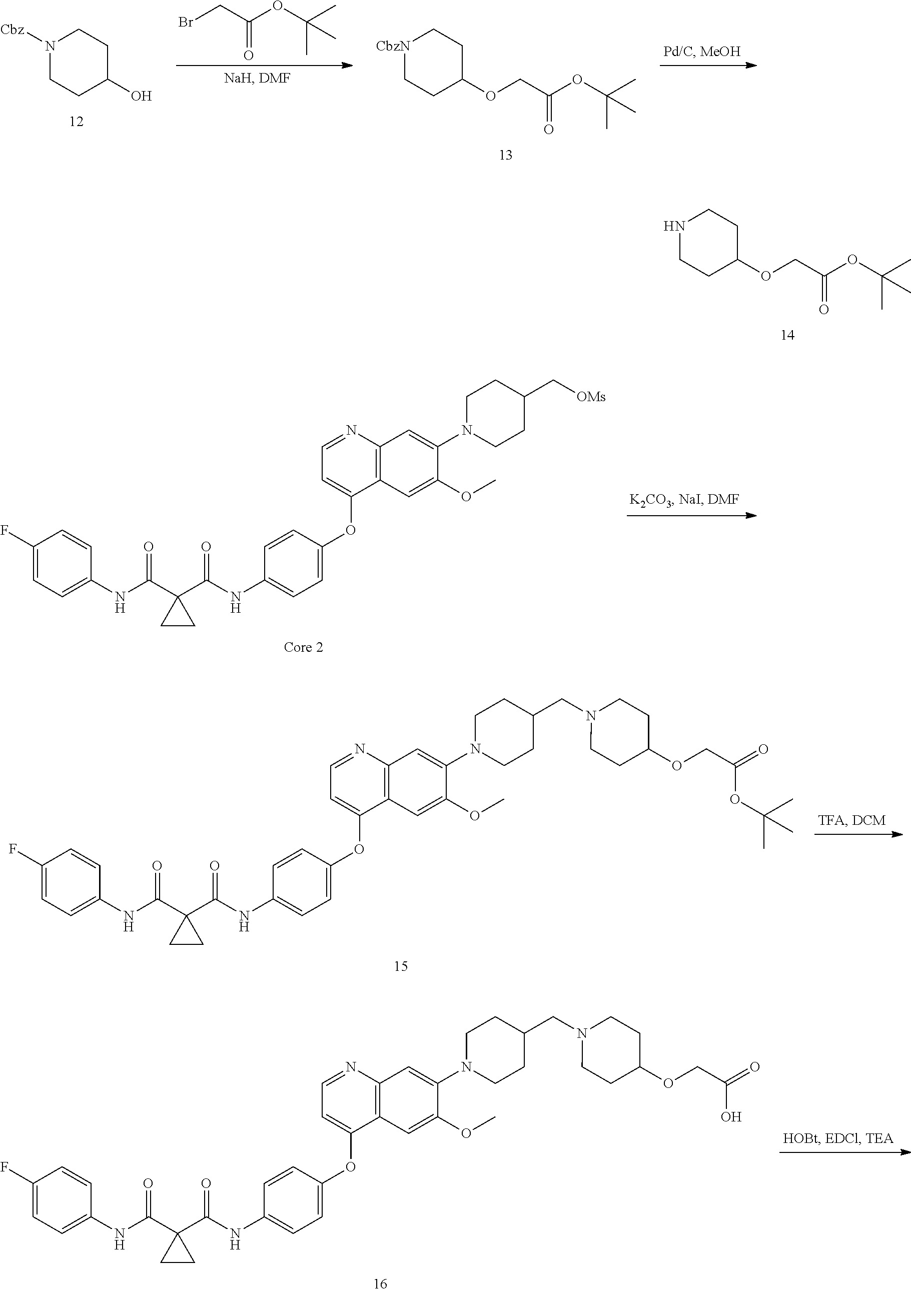

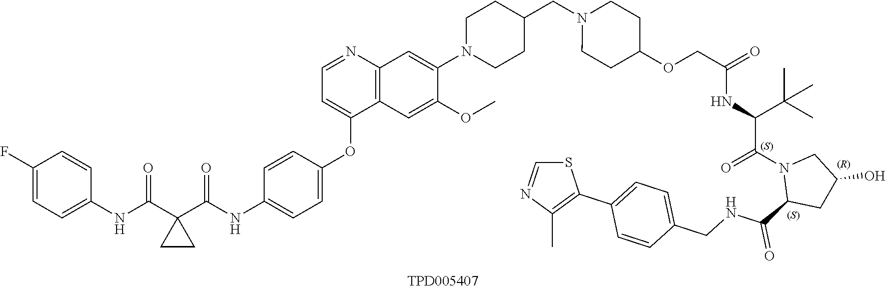

A DMF solution (100 ml) of compound 12 (10 g, 42.6 mmol) was taken and NaH (3.8 g, 112 mmol) was added at 0° C. and reacted at room temperature for 1 h. DMF solution (30 ml) of tert-butyl bromoacetate (12.4 g, 63.8 mol) was added to the above solution at room temperature and stirred at room temperature overnight. The reaction solution was poured into ammonium chloride solution (500 ml) quenched and extracted with ethyl acetate (500 ml×2). The combined organic phases were washed with water (500 ml) and dried over a column with anhydrous sodium sulfate to give compound 8 (5 g, 33%).

To a methanolic solution (50 ml) of compound 13 (5 g, 13.8 mmol), palladium carbon (1 g) was added and stirred overnight at room temperature in a hydrogen atmosphere (30 psi). The reaction solution was filtered and spun dry to give compound 14 (3 g, 80%).

A 100 ml single-necked vial was charged with compound core 1 (100 mg, 0.15 mmol), tert-butyl 2-(piperidin-4-acyloxy)acetate (64.5 mg, 0.30 mmol), potassium carbonate (83.0 mg, 0.60 mmol), sodium iodide (90.0 mg, 0.60 mmol) and N,N-dimethylformamide (10 ml). The reaction solution was stirred at 100° C. overnight. Quenched with water, extracted with ethyl acetate 3 times, the combined organic phases were washed with water 3 times, saturated brine once, dried over anhydrous sodium sulfate and spun-dried to give compound 15 (60 mg, yellow solid), yield: 50.8%.

To a 100 ml single mouth flask was added compound 7 (60 mg, 0.08 mmol), dichloromethane (10 ml) and trifluoroacetic acid (2 ml), the reaction solution was stirred at room temperature overnight, spun dry and pulped with methyl tert-butyl ether to give the crude compound 11 (40 mg, yellow solid).

A 100 ml triple-necked vial was charged with compound 16 (50 mg, 0.06 mmol), (2S,4R)-1-((S)-2-amino-3,3-dimethylbutanoyl)-4-hydroxy-N-(4-(4-methylthiazol-5-yl)benzyl)pyrrolidine-2-carboxamide hydrochloride (49 mg, 0.11 mmol) and triethylamine (21.3 mg. 0.2 mmol), dissolved with DCM (2 ml), 1-hydroxybenzotriazole (11.4 mg, 0.03 mmol) and 1-ethyl-(3-dimethylaminopropyl)carbodiimide hydrochloride (16.1 mg, 0.09 mmol) were added and stirred at room temperature overnight. Quenched with water, extracted with dichloromethane 3 times, the combined organic phases were washed with water 3 times, saturated brine once, dried over anhydrous sodium sulfate and spun dry. The crude product was purified by high pressure preparation to give compound TPD005407 (10 mg, yellow solid), yield: 15.9%. LCMS (ESI) m/z calcd. for C₆₂H₇₃FN₉O₉S [M+H]⁺ 1137.5; found 1137.3. The compound structure is confirmed.

### Example 10: Synthesis of Compound TPD005409

This embodiment was used to prepare the synthetic compound TPD005409 by the following synthetic route:

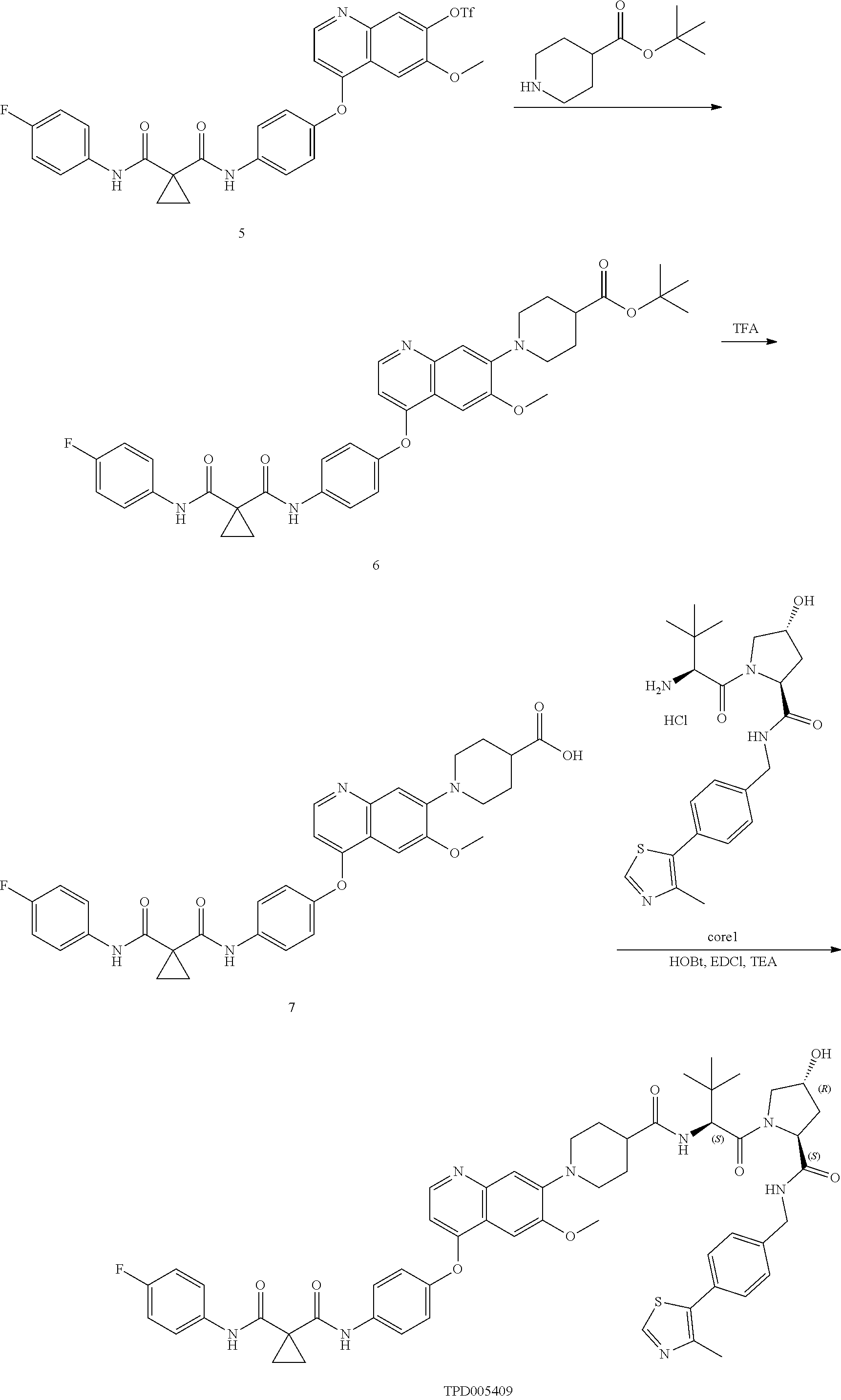

Compound 5 (200 mg, 0.32 mmol), tert-butyl piperidine-4-carboxylate (131.6 mg, 0.71 mmol), BINAP (80.4 mg, 0.13 mmol), Pd(dba)2 (55.7 mg, 0.10 mmol), and cesium carbonate (210.4 mg, 0.65 mmol), were dissolved in 1,4-dioxane (5 ml) and reacted at 100° C. overnight. After completion of the reaction the reaction solution was added to (30 ml) of water and extracted with EA (10 ml×3), the organic layer was dried and concentrated with anhydrous sodium sulfate and purified by preparative plate to give compound 6 as a yellow solid (50 mg, 23.6% yield).

Compound 6 (50 mg, 0.08 mmol) was dissolved in TFA (1 ml) and reacted overnight at room temperature. The reaction was completed vacuum spin drying the solvent of compound 7 yellow solid (50 mg, 109.4% yield).

Compound 7 (50 mg, 0.08 mmol), core1 (58.5 mg, 0.13 mmol), HOBT (13.5 mg, 0.10 mmol), EDCI (19.2 mg, 0.10 mmol), and triethylamine (25.4 mg, 0.25 mmol), were dissolved in DMF (2 ml), and the reaction was carried out at room temperature overnight. After completion of the reaction the reaction solution was added to (30 ml) of water and extracted with EA (10 ml×3), the organic layer was dried and concentrated with anhydrous sodium sulfate and purified by pre-HPLC to give the compound TPD005409 as a yellow solid (10.5 mg, yield 12.5%). LCMS (ESI) m/z calcd. for C₅₅H₆₀FN₈OS [M+H]⁺ 1011.4; found 1011.3. The compound structure is confirmed.

### Example 11: Synthesis of compound TPD005410

This embodiment was used to prepare the synthetic compound TPD005410 by the following synthetic route:

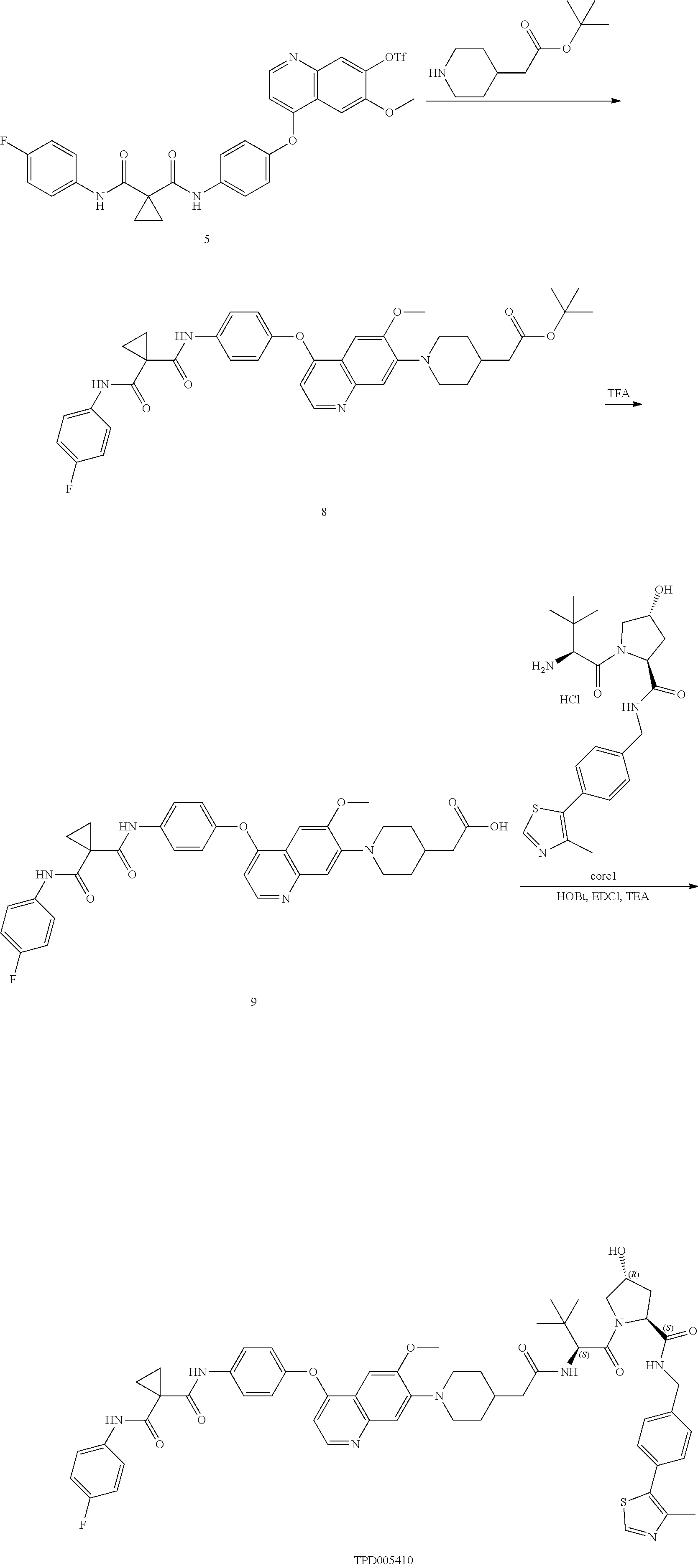

Compound 5 (200 mg, 0.32 mmol), tert-butyl 4-piperidineacetate oxalate (141.5 mg, 0.71 mmol), BINAP (80.4 mg, 0.13 mmol), Pd(dba)2 (55.7 mg, 0.10 mmol), and cesium carbonate (210.4 mg, 0.65 mmol), were dissolved in 1,4-dioxane (5 ml) and reacted at 100° C. overnight. After completion of the reaction the reaction solution was added to (30 ml) of water and extracted with EA (10 ml×3), the organic layer was dried and concentrated with anhydrous sodium sulfate and purified by preparative plate to give compound 8 as a yellow solid (50 mg, yield 23.2%).

Compound 8 (50 mg, 0.07 mmol) was dissolved in TFA (1 ml) and the reaction was carried out at room temperature overnight, the reaction was completed by vacuum spin drying the solvent of compound 9 yellow solid (50 mg).

Compound 9 (50 mg, 0.08 mmol), core1 (52.7 mg, 0.12 mmol), HOBT (13.2 mg, 0.10 mmol), EDCI (18.8 mg, 0.10 mmol), and triethylamine (24.8 mg, 0.24 mmol), were dissolved in DMF (2 ml), and the reaction was carried out at room temperature overnight. After completion of the reaction the reaction solution was added to (30 ml) of water and extracted with EA (10 ml×3), the organic layer was dried and concentrated with anhydrous sodium sulfate and purified by pre-HPLC to give the compound TPD005410 as a yellow solid (6.5 mg, yield 7.7%). LCMS (ESI) m/z calcd. for C₅₆H₆₂FN₈O₈S [M+H]⁺ 1024.4; found 1024.3. The compound structure is confirmed.

### Example 12: Synthesis of Compound TPD005411

This embodiment was used to prepare the synthetic compound TPD005411 by the following synthetic route:

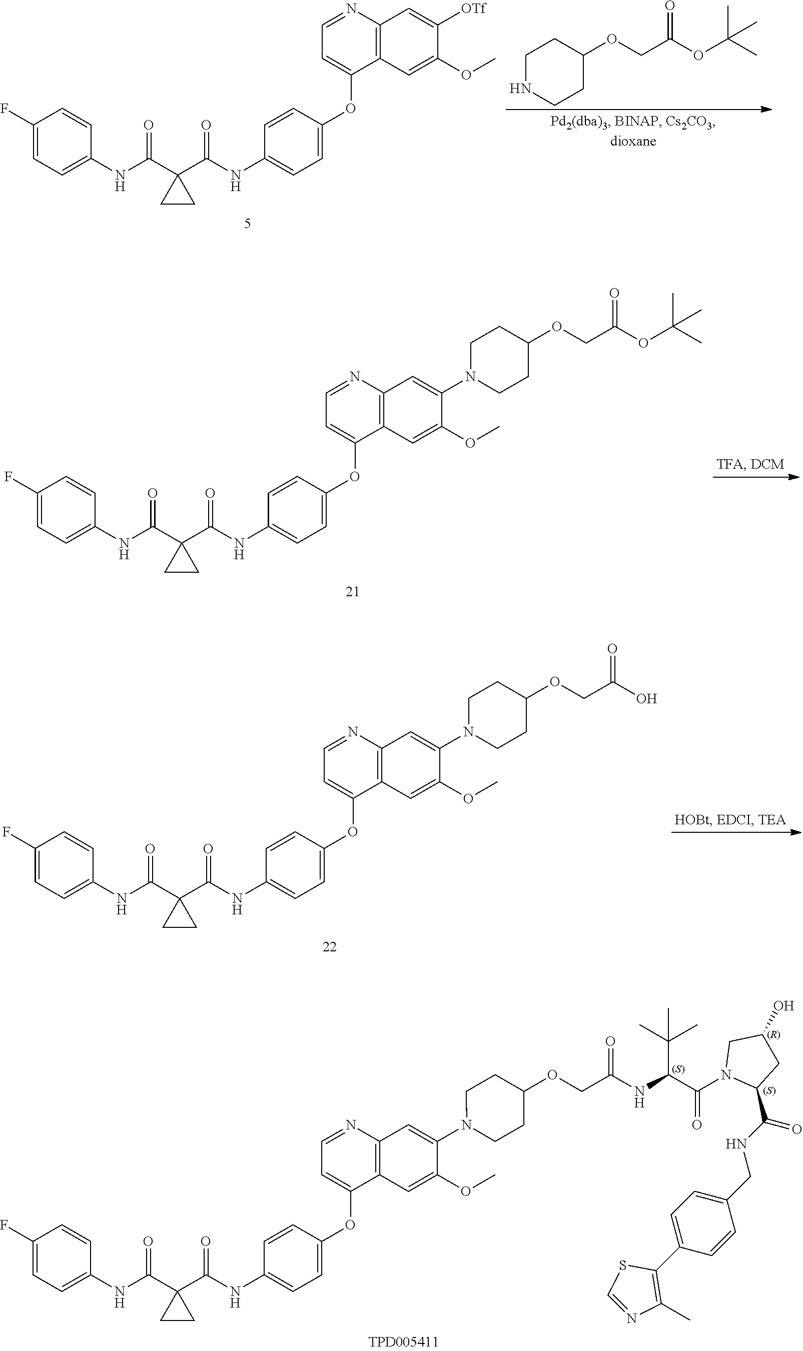

A 20 ml reaction vial was charged with compound 5 (100 mg, 0.16 mmol), tert-butyl 2-(piperidin-4-acyloxy)acetate (75.3 mg, 0.35 mmol), tris(dibenzylideneacetone)dipalladium (30.0 mg, 0.03 mmol), 1,1′-binaphthyl-2,2′-bisdiphenylphosphine (40.0 mg, 0.06 mmol), cesium carbonate (105 mg, 0.32 mmol) and 1,4-dioxane (10 ml), nitrogen displacement, and stirring at 100° C. overnight. Quenched with water, extracted with ethyl acetate 3 times, the combined organic phases were washed once with saturated brine, dried over anhydrous sodium sulfate and spun dry. The crude product was purified by column purification (petroleum ether/ethyl acetate=30:1-2:1) to give compound 21 (70.0 mg, yellow solid), yield: 63.6%.

To a 100 ml single necked vial was added compound 21 (70 mg, 0.10 mmol), dichloromethane (10 ml) and trifluoroacetic acid (2 ml), the reaction solution was stirred at room temperature overnight, spun dry and pulped with methyl tert-butyl ether to give the crude compound 22 (30 mg, yellow solid).

A 100 ml triple-necked vial was charged with compound 22 (30 mg, 0.05 mmol), (2S,4R)-1-((S)-2-amino-3,3-dimethylbutanoyl)-4-hydroxy-N-(4-(4-methylthiazol-5-yl)benzyl)pyrrolidine-2-carboxamide hydrochloride (44.2 mg, 0.1 mmol), and triethylamine (20.2 mg. 0.2 mmol), dissolved with DCM (2 ml), 1-hydroxybenzotriazole (27.2 mg, 0.2 mmol) and 1-ethyl-(3-dimethylaminopropyl)carbodiimide hydrochloride (38.4 mg, 0.2 mmol) were added and stirred at room temperature overnight. Quenched with water, extracted with dichloromethane 3 times, the combined organic phases were washed with water 3 times, saturated brine once, dried over anhydrous sodium sulfate and spun dry. The crude product was purified by high pressure preparation to give compound TPD005411 (10 mg, yellow solid), yield: 20%. LCMS (ESI) m/z calcd. for C₅₆H₆₂FN₈O₉S [M+H]⁺ 1040.4; found 1040.4. The compound structure is confirmed.

### Example 13: Synthesis of Compound TPD005412

This embodiment was used to prepare the synthetic compound TPD005412 by the following synthetic route:

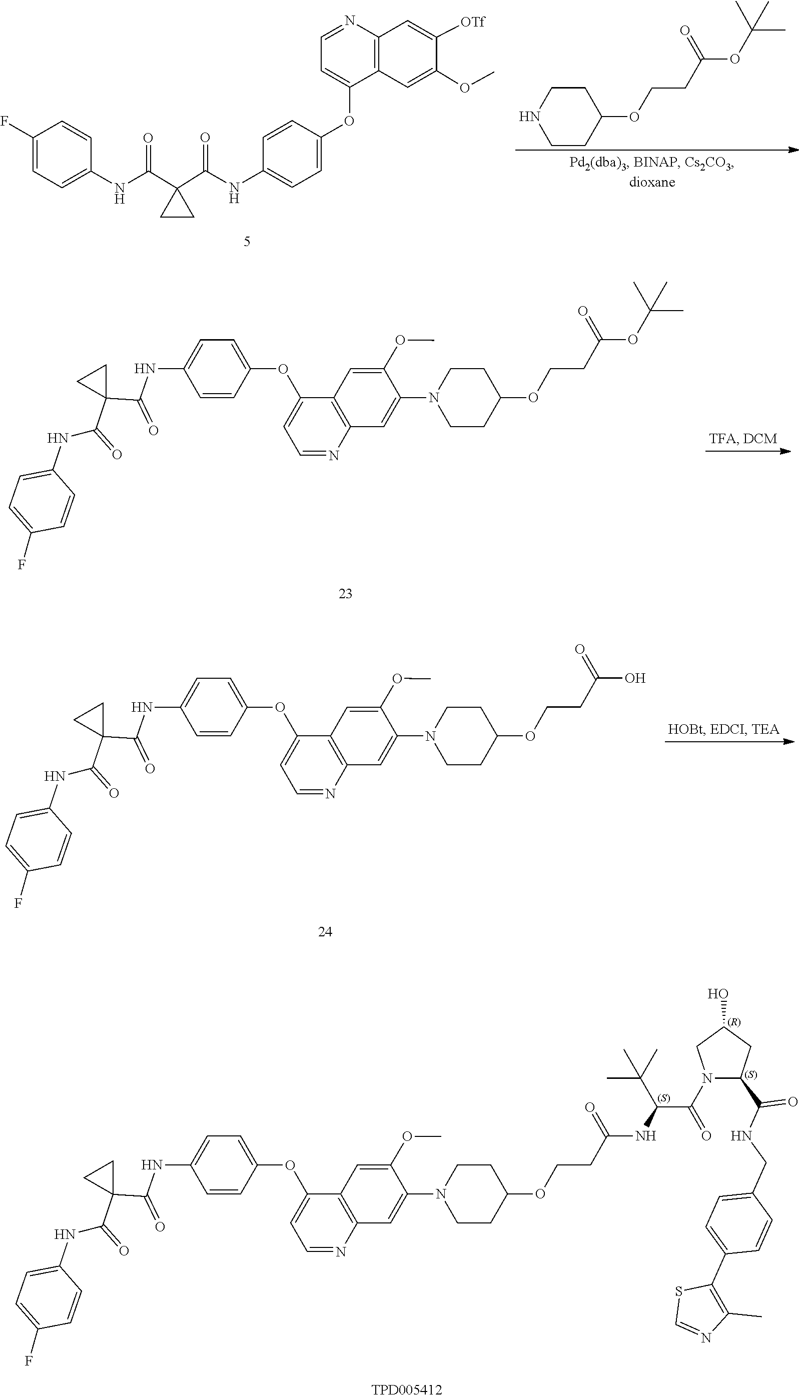

A 20 ml reaction vial was charged with compound 5 (100 mg, 0.16 mmol), tert-butyl 3-(piperidin-4-acyloxy)propionate (80 mg, 0.35 mmol), tris(dibenzylideneacetone)dipalladium (30.0 mg, 0.03 mmol), 1,1′-binaphthyl-2,2′-bis(diphenylphosphine) (40.0 mg, 0.06 mmol) Cesium carbonate (105 mg, 0.32 mmol) and 1,4-dioxane (10 ml), nitrogen displacement and stirring at 100° C. overnight. Quenched with water, extracted with ethyl acetate 3 times, the combined organic phases were washed once with saturated brine, dried over anhydrous sodium sulfate and spun dry. The crude product was purified by column purification (petroleum ether/ethyl acetate=30:1-2:1) to give compound 23 (80.0 mg, yellow solid), yield: 70.8%.

To a 100 ml single-necked vial was added compound 23 (80 mg, 0.11 mmol), dichloromethane (10 ml) and trifluoroacetic acid (2 ml), the reaction solution was stirred at room temperature overnight, spun dry, and pulped with methyl tert-butyl ether to give the crude compound 22 (30 mg, yellow solid).

A 100 ml triple-necked vial was charged with compound 24 (30 mg, 0.05 mmol), (2S,4R)-1-((S)-2-amino-3,3-dimethylbutanoyl)-4-hydroxy-N-(4-(4-methylthiazol-5-yl)benzyl)pyrrolidine-2-carboxamide hydrochloride (44.2 mg, 0.1 mmol), and triethylamine (20.2 mg. 0.2 mmol), dissolved with DCM (2 ml), 1-hydroxybenzotriazole (27.2 mg, 0.2 mmol) and 1-ethyl-(3-dimethylaminopropyl)carbodiimide hydrochloride (38.4 mg, 0.2 mmol) were added and stirred at room temperature overnight. Quenched with water, extracted with dichloromethane 3 times, the combined organic phases were washed with water 3 times, saturated brine once, dried over anhydrous sodium sulfate and spun dry. The crude product was purified by high pressure preparation to give compound TPD005412 (10 mg, yellow solid), yield: 20%. LCMS (ESI) m/z calcd. for C₅₇H₆₄FN₈O₉S [M+H]⁺ 1054.4; found 1054.3. The compound structure is confirmed.

### Example 14: Synthesis of Compound TPD005415

This embodiment was used to prepare the synthetic compound TPD005415 by the following synthetic route:

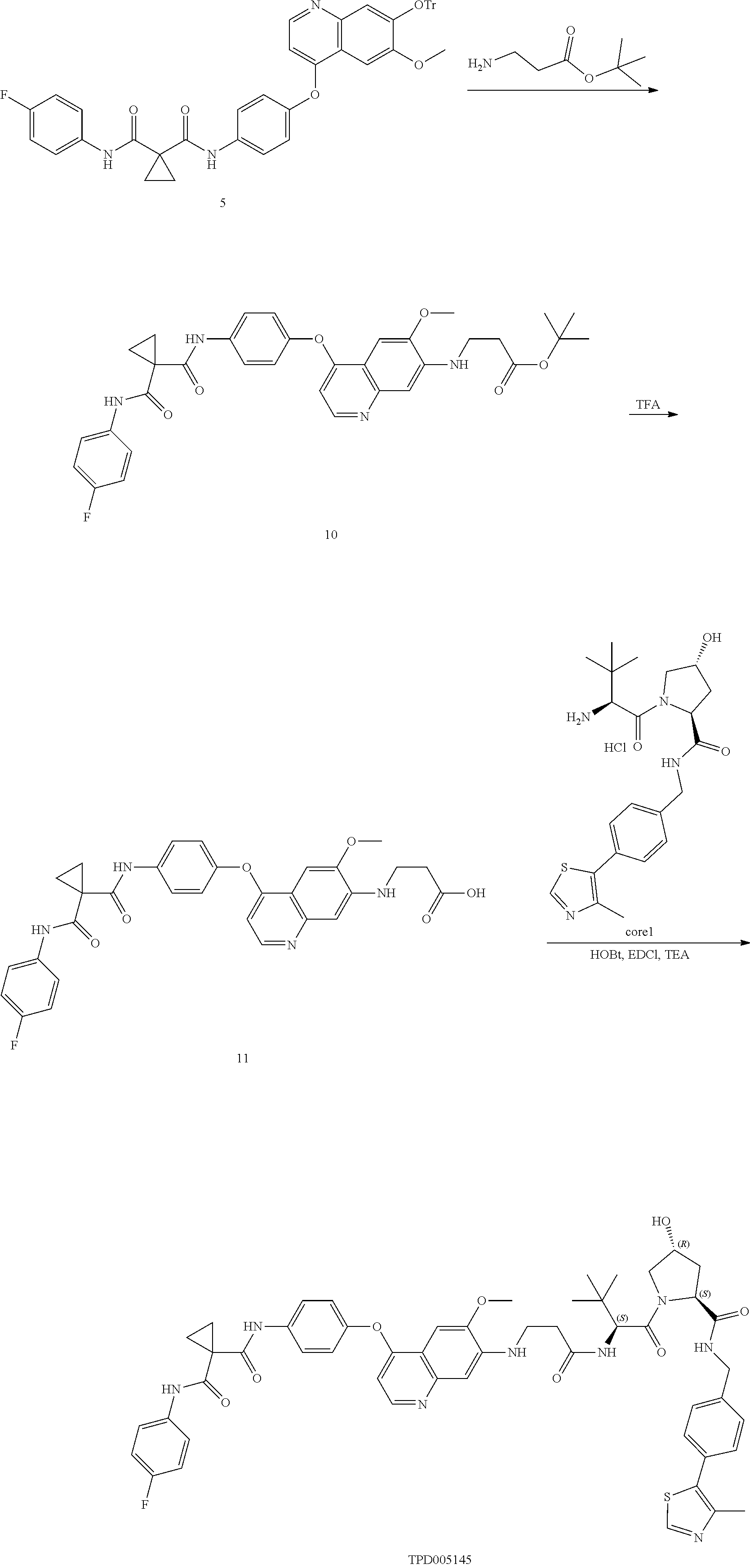

Compound 5 (200 mg, 0.32 mmol), tert-butyl 3-aminopropionate (103.1 mg, 0.71 mmol), BINAP (80.4 mg, 0.13 mmol), Pd(dba)2 (55.7 mg, 0.10 mmol) and cesium carbonate (210.4 mg, 0.65 mmol) were dissolved in 1,4-dioxane (5 ml) and reacted at 100° C. overnight. After completion of the reaction the reaction solution was added to (30 ml) of water and extracted with EA (10 ml×3), the organic layer was dried and concentrated with anhydrous sodium sulfate and purified by preparative plate to give compound 8 as a yellow solid (50 mg, yield 25.2%).

Compound 10 (50 mg, 0.07 mmol) was dissolved in TFA (1 ml) and reacted overnight at room temperature. The reaction was completed vacuum spin drying the solvent of compound 11 yellow solid (50 mg, yield 110.1%).

Compound 11 (50 mg, 0.09 mmol), core1 (57.8 mg, 0.13 mmol), HOBT (14.5 mg, 0.11 mmol), EDCI (20.6 mg, 0.11 mmol), and triethylamine (27.2 mg, 0.27 mmol), were dissolved in DMF (2 ml), and the reaction was carried out at room temperature overnight. After completion of the reaction the reaction solution was added to (30 ml) of water and extracted with EA (10 ml×3), the organic layer was dried and concentrated with anhydrous sodium sulfate and purified by pre-HPLC to give the compound TPD005415 as a yellow solid (11 mg, yield 12.7%). LCMS (ESI) m/z calcd. for C₅₂H₅₆FN₈O₈S [M+H]⁺ 970.4; found 970.3. The compound structure is confirmed.

### Example 15: Synthesis of Compound TPD005417

This embodiment was used to prepare the synthetic compound TPD005417 by the following synthetic route:

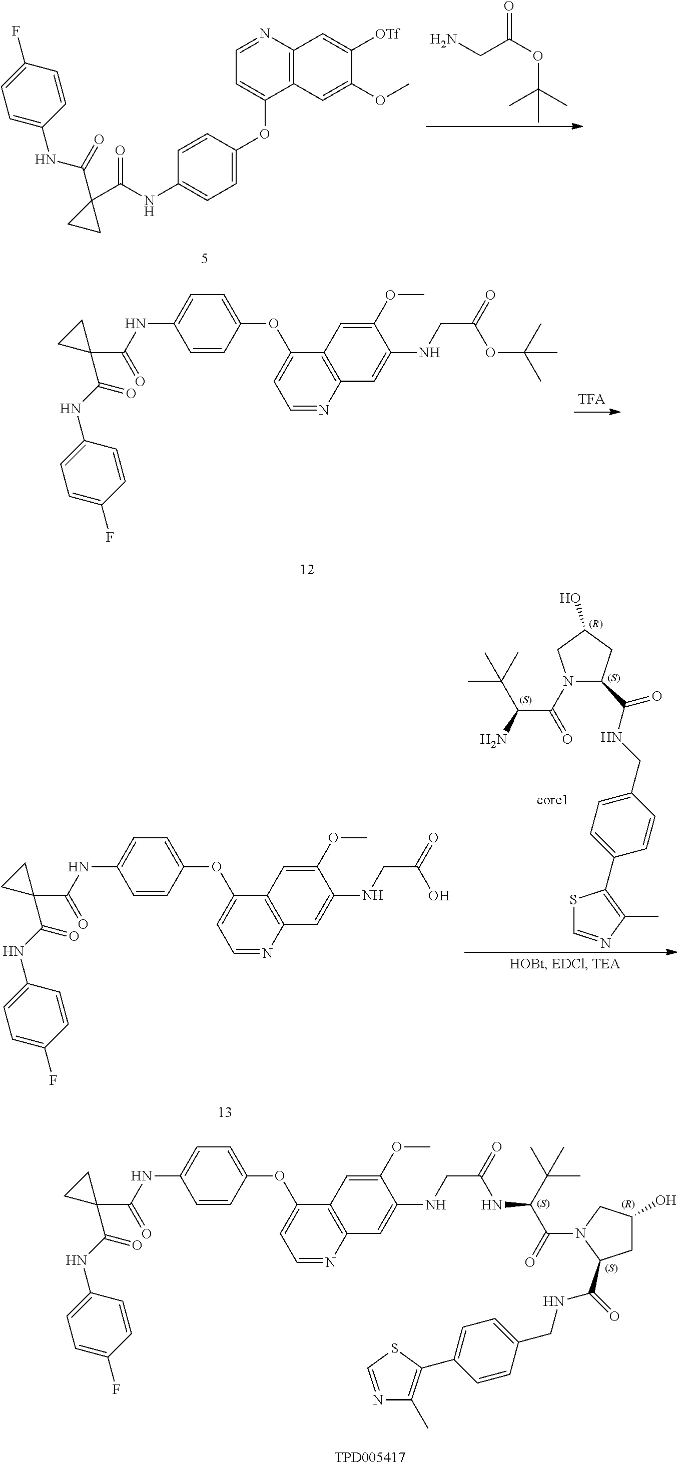

Compound 5 (200 mg, 0.32 mmol), tert-butylglycinate (93.2 mg, 0.71 mmol), BINAP (80.4 mg, 0.13 mmol), Pd(dba)₂ (55.7 mg, 0.10 mmol) and cesium carbonate (210.4 mg, 0.65 mmol) were dissolved in 1,4-dioxane (5 ml) in 100° C. and reacted overnight. After completion of the reaction the reaction solution was added to (30 ml) of water and extracted with EA (10 ml×3), the organic layer was dried and concentrated with anhydrous sodium sulfate and purified by preparative plate to give compound 12 as a yellow solid (50 mg, yield 25.8%).

Compound 12 (50 mg, 0.08 mmol) was dissolved in TFA (1 ml) and reacted overnight at room temperature. The reaction was completed vacuum spin drying the solvent of compound 13 yellow solid (50 mg, yield 110.3%).

Compound 13 (50 mg, 0.09 mmol), core1 (59.3 mg, 0.14 mmol), HOBT (14.9 mg, 0.11 mmol), EDCI (21.1 mg, 0.11 mmol) and triethylamine (27.9 mg, 0.28 mmol) were dissolved in DMF (2 ml), and the reaction was carried out at room temperature overnight. After completion of the reaction the reaction solution was added to (30 ml) of water and extracted with EA (10 ml×3), the organic layer was dried and concentrated with anhydrous sodium sulfate and purified by pre-HPLC to give the compound TPD005417 as a yellow solid (10.1 mg, yield 11.5%). LCMS (ESI) m/z calcd. for C₅₁H₅₄FN₈O₈S [M+H]⁺ 956.4; found 956.3. The compound structure is confirmed.

### Example 16: Synthesis of Compound TPD005420

This embodiment was used to prepare the synthetic compound TPD005420 by the following synthetic route:

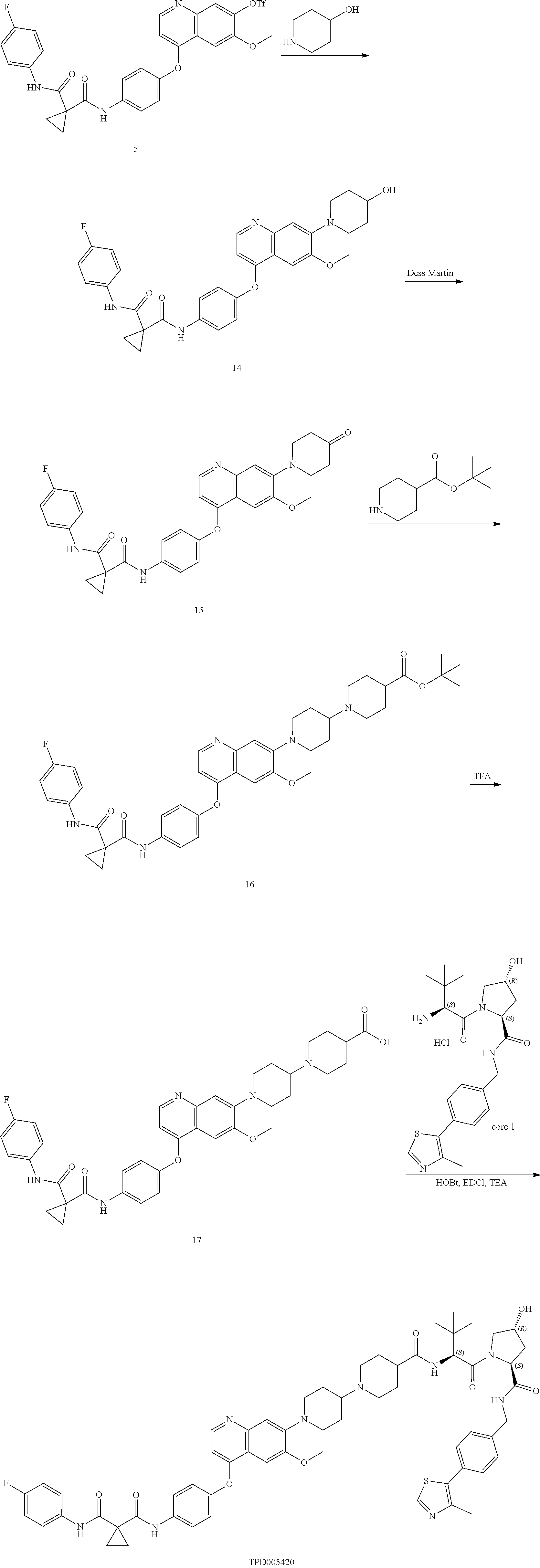

Compound 5 (200 mg, 0.32 mmol), 4-hydroxypiperidine (49.0 mg, 0.48 mmol), BINAP (80.4 mg, 0.13 mmol), Pd(dba)₂ (55.7 mg, 0.10 mmol) and cesium carbonate (210.4 mg, 0.65 mmol) were dissolved in 1,4-dioxane (5 ml) in 100° C. and reacted overnight. After completion of the reaction the reaction solution was added to (30 ml) of water and extracted with EA (10 ml×3), the organic layer was dried and concentrated with anhydrous sodium sulfate and purified by preparative plate to give compound 14 as a yellow solid (50 mg, 27.1% yield).

Compound 14 (250 mg, 0.44 mmol) was dissolved in DCM (10 ml), Des Martin (371.6 mg, 0.88 mmol) was added in batches and the reaction was carried out at room temperature overnight. After completion of the reaction the reaction solution was added to (30 ml) of water and extracted with DCM (10 ml×3), the organic layer was dried and concentrated with anhydrous sodium sulfate and purified by preparative plate to give compound 15 as a yellow solid (150 mg, yield 60.2%).

Compound 15 (150 mg, 0.26 mmol) and tert-butyl piperidine-4-carboxylate (146.6 mg, 0.79 mmol) were dissolved in DCM (5 ml) and tetraisopropyl titanate (1 ml), stirred at room temperature for 3 h. Sodium borohydride acetate (167.9 mg, 0.79 mmol) was added, and the reaction was carried out at room temperature overnight. After completion of the reaction the reaction solution was added to (30 ml) of water and extracted with DCM (10 ml×3), the organic layer was dried and concentrated with anhydrous sodium sulfate and purified by preparative plate to give compound 16 as a yellow solid (40 mg, 20.5% yield).

Compound 12 (70 mg, 0.09 mmol) was dissolved in TFA (1 ml) and reacted overnight at room temperature. The reaction was completed vacuum spin drying the solvent of compound 13 yellow solid (70 mg, 108.2% yield).

Compound 17 (70 mg, 0.10 mmol), core1 (71.8 mg, 0.15 mmol), HOBT (16.7 mg, 0.12 mmol), EDCI (23.6 mg, 0.12 mmol) and triethylamine (31.2 mg, 0.31 mmol) were dissolved in DMF (2 ml), and the reaction was carried out at room temperature overnight. After completion of the reaction the reaction solution was added to (30 ml) of water and extracted with EA (10 ml×3), the organic layer was dried and concentrated with anhydrous sodium sulfate and purified by pre-HPLC to give the compound TPD005420 as a yellow solid (10.3 mg, 9.1% yield). LCMS (ESI) m/z calcd. for C₆₀H₆₉FN₉O₈S [M+H]⁺ 1094.5; found 1094.3. The compound structure is confirmed.

### Example 17: Synthesis of Compound TPD005421

This embodiment was used to prepare the synthetic compound TPD005421 by the following synthetic route:

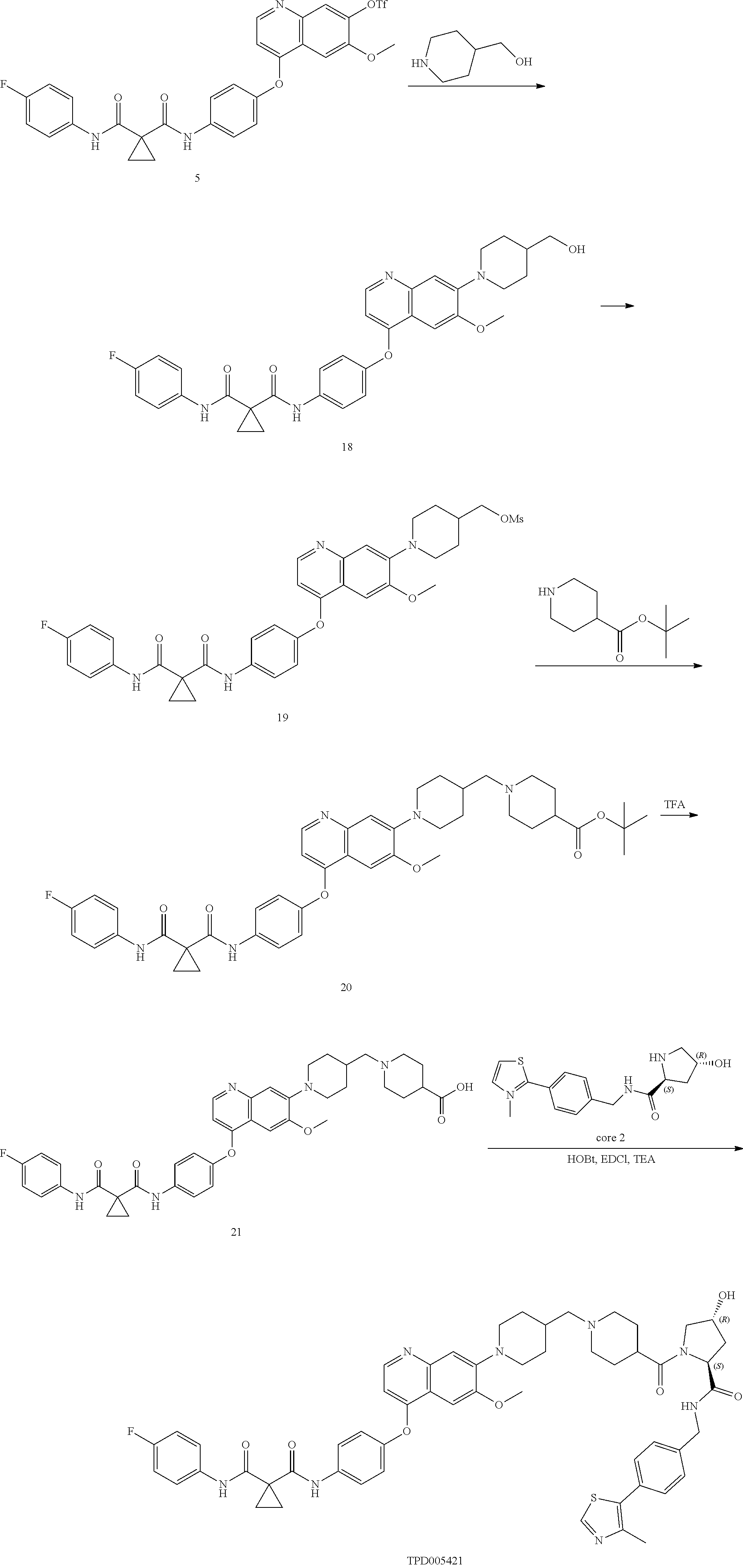

Compound 5 (200 mg, 0.32 mmol), 4-hydroxypiperidine (81.8 mg, 0.71 mmol), BINAP (80.4 mg, 0.13 mmol), Pd(dba)₂ (55.7 mg, 0.10 mmol) and cesium carbonate (210.4 mg, 0.65 mmol) were dissolved in 1,4-dioxane (5 ml) in 100° C. and reacted overnight. After completion of the reaction the reaction solution was added to (30 ml) of water and extracted with EA (10 ml×3), the organic layer was dried and concentrated with anhydrous sodium sulfate and purified by preparative plate to give compound 14 as a yellow solid (50 mg, yield 26.5%).

Compound 18 (500 mg, 0.86 mmol) and triethylamine (129.8 mg, 1.28 mmol) were dissolved in DCM (5 ml), MsCl (117.6 mg, 1.03 mmol) was added dropwise at 0° C. and the reaction was carried out at room temperature for 2 hours. After completion of the reaction the reaction solution was washed with water (2 ml) and the organic phase was dried with anhydrous sodium sulfate and concentrated to give compound 19 as a yellow solid (500 mg, yield 88.2%).

Compound 19 (300 mg, 0.45 mmol), tert-butyl piperidine-4-carboxylate (125.8 mg, 0.68 mmol), sodium iodide (135.8 mg, 0.91 mmol) and potassium carbonate (187.7 mg, 1.36 mmol) were dissolved in DMF (5 ml) and stirred at 80° C. overnight. After completion of the reaction the reaction solution was added to (30 ml) of water and extracted with EA (10 ml×3), the organic layer was dried and concentrated with anhydrous sodium sulfate and purified by preparative plate to give compound 20 as a yellow solid (200 mg, 58.8% yield).

Compound 20 (200 mg, 0.27 mmol) was dissolved in TFA (2 ml) and reacted overnight at room temperature. The reaction was completed vacuum spin drying the solvent of compound 21 yellow solid (150 mg, yield 69.7%).
compound 21 (100 mg, 0.14 mmol), the aforementioned compound core2 (68.4 mg, 0.22 mmol), HOBT (23.3 mg, 0.17 mmol), EDCI (33.1 mg, 0.17 mmol), and triethylamine (43.6 mg, 0.43 mmol) were dissolved in DMF (2 ml) at room temperature and the The reaction was carried out overnight. After completion of the reaction the reaction solution was added to (30 ml) of water and extracted with EA (10 ml×3), the organic layer was dried and concentrated with anhydrous sodium sulfate and purified by pre-HPLC to give the compound TPD005421 as a yellow solid (12.1 mg, 7.6% yield). LCMS (ESI) m/z calcd. for C₅₅H₅₉FN₈O₇S [M+H]⁺ 994.4; found 994.3. The compound structure is confirmed.

### Example 18: Synthesis of compound TPD005423

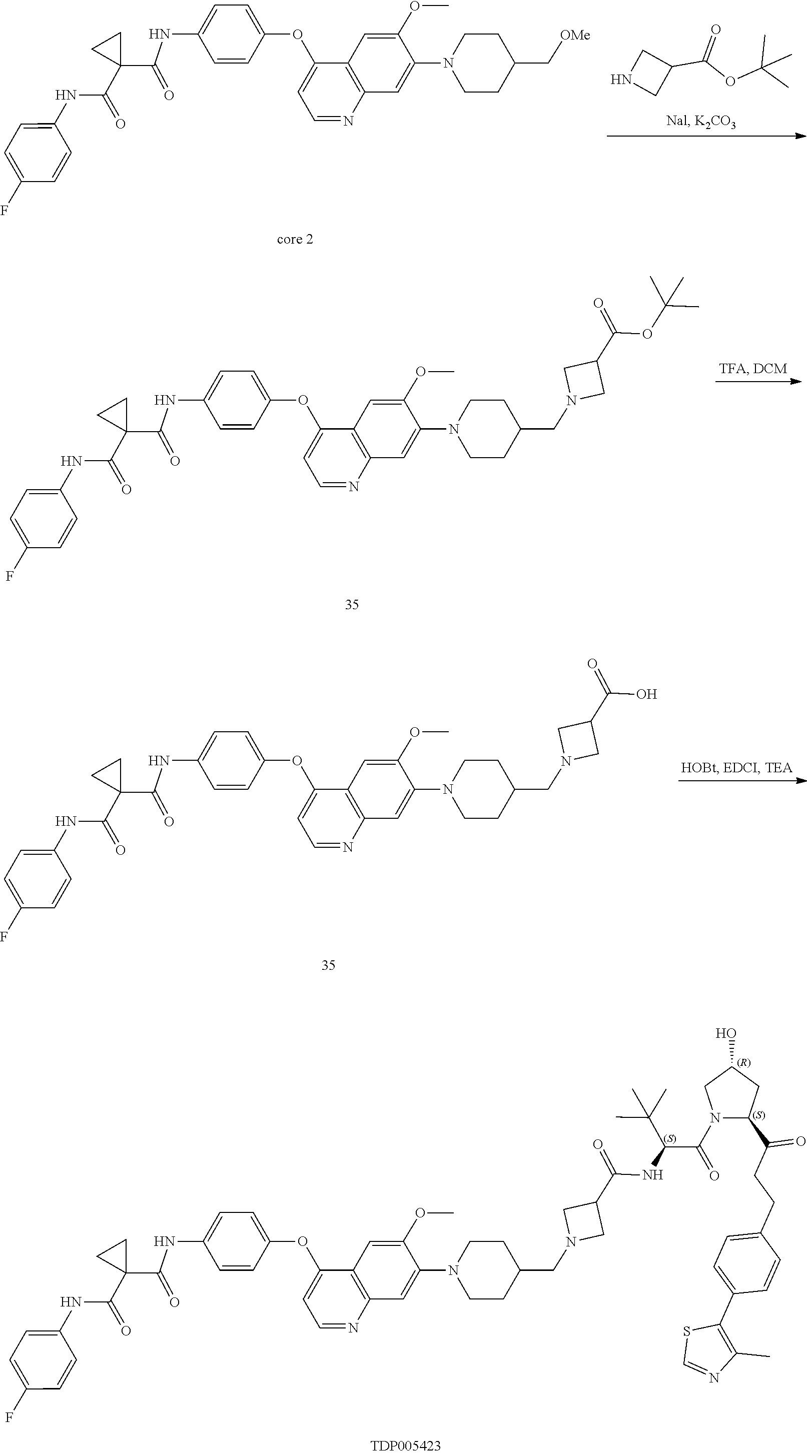

A 100 ml single-necked vial was charged with compound core 1 (300 mg, 0.45 mmol), tert-butyl azetidine-3-carboxylate (94.2 mg, 0.6 mmol), potassium carbonate (83.0 mg, 0.60 mmol), sodium iodide (90.0 mg, 0.60 mmol), N,N-dimethylformamide (10 ml). The reaction solution was stirred at 100° C. overnight. Quenched with water, extracted with ethyl acetate 3 times, the combined organic phases were washed with water 3 times, saturated brine once, dried over anhydrous sodium sulfate and spun-dried to give compound 7 (120 mg, yellow solid), yield: 36.5%.

To a 40 ml single-necked vial was added compound 35 (120 mg, 0.17 mmol), dichloromethane (10 ml) and trifluoroacetic acid (2 ml), the reaction solution was stirred at room temperature overnight, spun dry, and pulped with methyl tert-butyl ether to give the crude compound 36 (100 mg, yellow solid).

A 100 ml triple-necked vial was charged with compound 8 (100 mg, 0.15 mmol), (2S,4R)-1-((S)-2-amino-3,3-dimethylbutanoyl)-4-hydroxy-N-(4-(4-methylthiazol-5-yl)benzyl)pyrrolidine-2-carboxamide hydrochloride (124 mg, 0.3 mmol) and triethylamine (50.5 mg. 0.5 mmol), dissolved with DCM (5 ml), 1-hydroxybenzotriazole (114 mg, 0.25 mmol) and 1-ethyl-(3-dimethylaminopropyl)carbodiimide hydrochloride (161 mg, 0.85 mmol) were added and stirred at room temperature overnight. Quenched with water, extracted with dichloromethane 3 times, the combined organic phases were washed with water 3 times, saturated brine once, dried over anhydrous sodium sulfate and spun dry. The crude product was purified by high pressure preparation to give compound TPD005423 (10 mg, yellow solid), yield: 6.2%. LCMS (ESI) m/z calcd. for C₅₉H₆₇FN₉O₈S [M+H]⁺ 1079.5; found 1079.4. The compound structure is confirmed.

### Example 19: Synthesis of Compound TPD005424

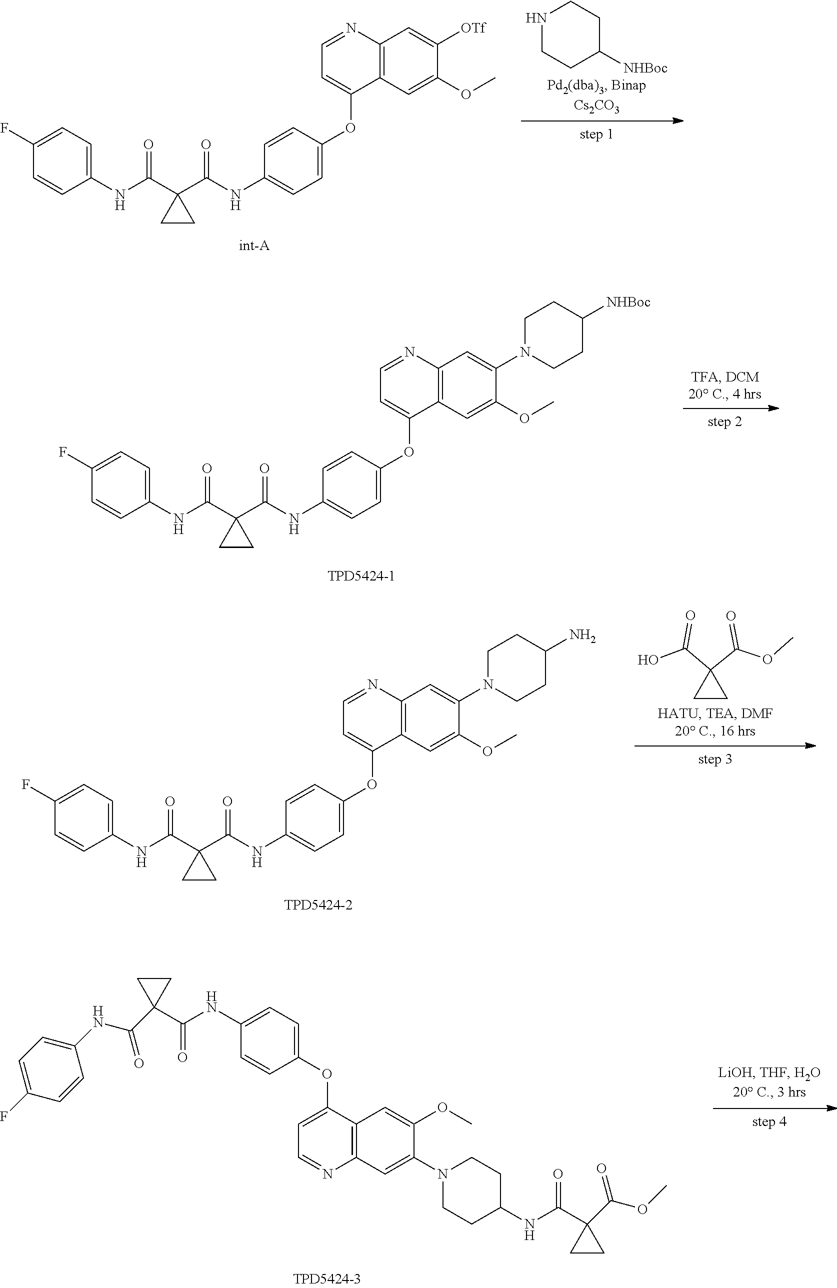

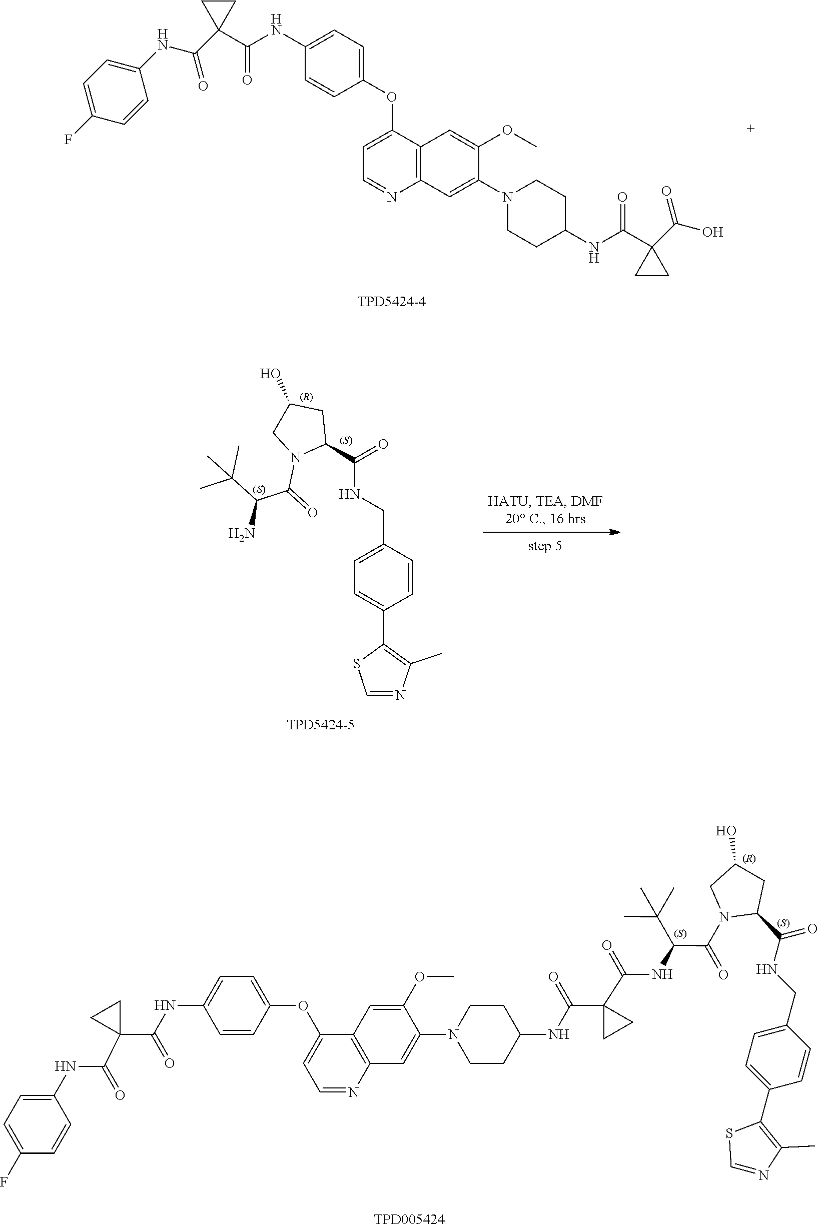

To a 100 ml triple-necked vial were sequentially added compound int-A (600 mg, 0.9685 mmol), 4-Boc-aminopiperidine (387.94 mg, 1.937 mmol), cesium carbonate (631.11 mg, 1.937 mmol), BINAP (241.22 mg, 0.3874 mmol), Pd₂ (dba)₃ (177.37 mg, 0.1937 mmol) and 1,4-dioxane (30 ml). The reaction was carried out under nitrogen protection at 100° C. for 16 hours. After the reaction was completed, the reaction solution was reduced to room temperature and concentrated to dryness, and the residue was purified by silica gel column with eluent ratio of DCM/MeOH=50/1˜30/1 to obtain the compound TPD5424-1 (440 mg, yellow solid, purity 84.226%), yield: 57.13%. LCMS (ESI) m/z calcd. for C₃₇H₄₀FN₅O₆ [M+H]⁺ 670.3; found 670.2. ¹H, NMR (400 MHz, CDCl₃): δ=9.34 (s, 1H), 8.81 (s, 1H), 8.46 (d, J=5.3 Hz, 1H), 7.62 (d, J=8.9 Hz, 2H), 7.52-7.45 (m, 4H), 7.16 (d, J=8.9 Hz, 2H), 7.05 (t, J=8.6 Hz, 2H), 6.43 (d, J=5.2 Hz, 1H), 4.52 (s, 1H), 4.02 (s, 3H), 3.64 (d, J=12.1 Hz, 2H), 2.83 (t, J=11.0 Hz, 2H), 2.11 (d, J=10.9 Hz, 2H). 1.74-1.71 (m, 2H), 1.68-1.65 (m, 4H), 1.47 (s, 9H).

A 50 ml three-necked vial was charged with compound TPD5424-1 (440 mg, 0.657 mmol), DCM (8 ml) and TFA (4 ml). The reaction solution reacted at 20° C. for 4 hours. After completion of the reaction the reaction solution was directly spun dry to give the crude compound TPD5424-2 (440 mg, yellow solid, purity 91.595%), yield: 89.73%. LCMS (ESI) m/z calcd. for C₃₂H₃₂FN₅O₄ [M+H]⁺ 570.2; found 570.2. The compound structure is confirmed.

To a 50 ml triple-necked vial was sequentially added compound TPD5424-2 (300 mg, 0.563 mmol), monomethyl 1,1-cyclopropyl dicarboxylate (91 mg, 0.632 mmol), DMF (5 ml), HATU (300 mg, 0.790 mmol) and triethylamine (266 mg, 2.633 mmol). The reaction mixture reacted at 20° C. for 16 hours. After completion of the reaction the reaction solution was poured into saturated aqueous sodium bicarbonate solution (20 ml) and the aqueous phase was extracted three times with DCM (20 ml). The organic phases were combined, washed with saturated brine, dried over anhydrous sodium sulfate, filtered, and concentrated to dryness. The residue was purified by silica gel column with eluent ratio of DCM/MeOH=50/1˜30/1 to afford the compound TPD5424-3 (250 mg, yellow solid, purity 97.596%), yield: 66.58%. LCMS (ESI) m/z calcd. for C₃₇H₃₆FN₄O₇ [M+H]⁺ 696.3; found 696.1. ¹H NMR (400 MHz, CDCl₃): δ=9.49 (s, 1H), 8.88 (d, J=7.8 Hz, 1H), 8.76 (s, 1H), 8.43 (d, J=5.6 Hz, 1H), 7.69-7.63 (m, 2H), 7.53 (s, 1H), 7.49-7.46 (m, 3H). 7.20-7.15 (m, 2H), 7.09-7.01 (m, 2H), 6.48 (d, J=5.6 Hz, 1H), 4.05 (s, 3H), 3.70-3.62 (m, 5H), 2.96 (t, J=10.4 Hz, 2H), 2.13-2.11 (m, 2H), 1.88-1.64 (m, 8H). 1.59-1.56 (m, 2H). The compound structure is confirmed.

To a 50 ml three-necked vial was added sequentially the compound TPD5424-3 (100 mg, 0.1437 mmol), THF (2 ml), H₂O (1 ml) and lithium hydroxide monohydrate (12.06 mg, 0.2874 mmol). the reaction was carried out at 20° C. for 3 h. The reaction solution was adjusted to pH 8 with dilute hydrochloric acid, then the mixture was extracted three times with ethyl acetate (5 ml), anhydrous sodium sulfate. The reaction solution was adjusted to pH 8 with dilute hydrochloric acid, then the mixture was extracted three times with ethyl acetate (5 ml), the combined organic phases were washed with saturated brine, dried over anhydrous sodium sulfate, filtered, and spun-dried to give the crude compound, TPD5424-5 (50 mg, yellow solid, purity 97.614%), yield: 49.83%. LCMS (ESI) m/z calcd. for C₃₇H₃₆FN₅O₇ [M+H]⁺ 682.26; found 682.2. The compound structure is confirmed.

A 50 ml triple-necked vial was sequentially charged with compound TPD5424-4 (50 mg, 0.0733 mmol), compound TPD5424-5 (34.72 mg, 0.0806 mmol), DMF (4 ml), HATU (41.81 mg, 0.1099 mmol) and triethylamine (22.25 mg, 0.2199 mmol). The reaction mixture reacted at 20° C. for 16 hours. After completion of the reaction the reaction solution was poured into saturated aqueous sodium bicarbonate solution (20 ml) and the aqueous phase was extracted three times with DCM (20 ml). The organic phases were combined, washed with saturated brine, dried over anhydrous sodium sulfate, filtered, and concentrated to dryness. The residue was purified by preparative plate with unfolding agent ratio DCM/MeOH=10/1 to give compound TPD005424 (26.6 mg, off-white solid, purity 97.557%), yield: 32.33%. LCMS (ESI) m/z calcd. for C₅₉H₆₄ FN₉O₉S [M+H]⁺ 1094.45; found 1094.7. ¹H NMR (400 MHz, DMSO-d₆): δ=10.19 (s, 1H), 10.06 (s, 1H), 9.10 (d, J=8.6 Hz, 1H), 8.96 (s, 1H), 8.68 (t, J=5.9 Hz, 1H), 8.44 (d, J=5.2 Hz, 1H), 7.74 (dd, J=18.3 8.3 Hz, 3H), 7.67-7.59 (m, 2H), 7.46 (s, 1H), 7.44-7.36 (m, 4H), 7.34 (s, 1H), 7.21 (d, J=9.0 Hz, 2H), 7.15 (dd, J=12.3, 5.5 Hz, 2H), 6.40 (d, J=5.2 Hz, 1H). 5.15 (d, J=3.5 Hz, 1H), 4.58-4.17 (m, 5H), 3.99-3.76 (m, 4H), 3.72-3.47 (m, 4H), 2.86-2.66 (m, 2H), 2.43 (s, 3H), 2.04 (d, J=8.5 Hz, 1H), 1.96-1.65 (m, 5H). 1.47 (s, 4H), 1.29 (s, 4H), 0.95 (s 9H). The compound structure is confirmed.

### Example 20: Synthesis of Compound TPD005430

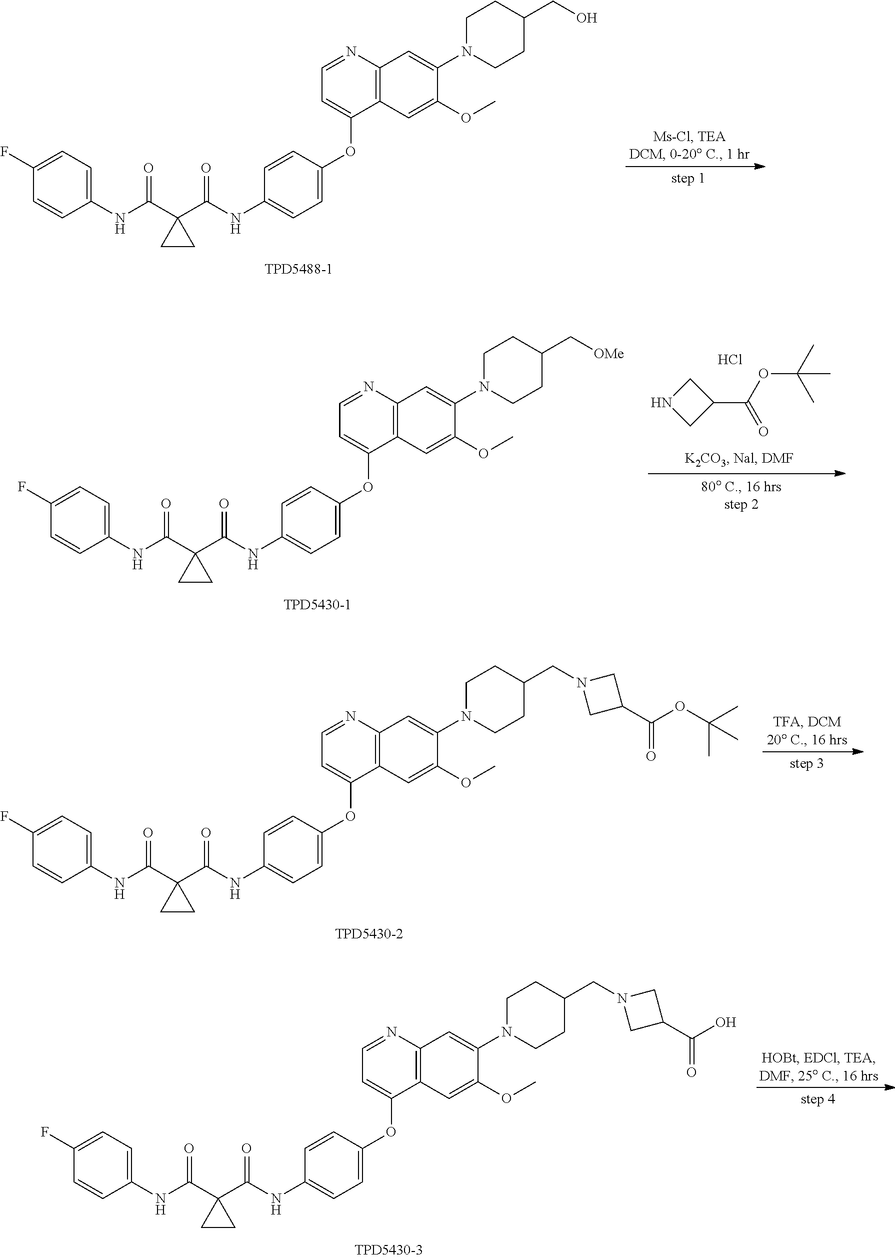 [SMILES: COc1cc2c(Oc3ccc(NC(=O)C4(C(=O)Nc5ccc(F)cc5)CC4)cc3)ccnc2cc1N1CCC(CO)CC1 ; COCC1CCN(c2cc3nccc(Oc4ccc(NC(=O)C5(C(=O)Nc6ccc(F)cc6)CC5)cc4)c3cc2OC)CC1 ; CC(C)(C)OC(=O)C1CNC1 ; Cl ; COc1cc2c(Oc3ccc(NC(=O)C4(C(=O)Nc5ccc(F)cc5)CC4)cc3)ccnc2cc1N1CCC(CN2CC(C(=O)OC(C)(C)C)C2)CC1 ; COc1cc2c(Oc3ccc(NC(=O)C4(C(=O)Nc5ccc(F)cc5)CC4)cc3)ccnc2cc1N1CCC(CN2CC(C(=O)O)C2)CC1]

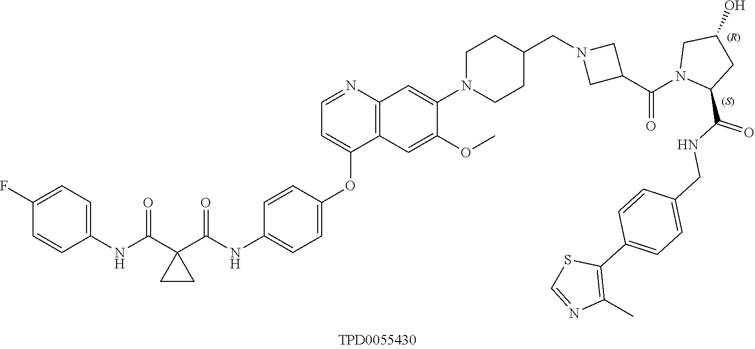

Compound TPD5488-1 (200 mg, 0.3421 mmol), triethylamine (103.85 mg, 1.0263 mmol) and dichloromethane (10 ml) were added sequentially to a 50 ml triple-necked vial. Cooled down under nitrogen protection to 0° C. The nmethylsulfonyl chloride (58.78 mg, 0.51315 mmol) was added dropwise. After dropwise addition, the cold bath was removed and the reaction was naturally warmed to 20° C. for 1 hour. The reaction solution was decanted into water (20 ml) and extracted three times with DCM (5 ml). The organic phases were combined, washed with saturated brine, dried over anhydrous sodium sulfate, filtered, and concentrated to dryness to obtain the crude compound TPD5430-1 (220 mg, yellow solid, purity 83.913%), yield: 81.44%. LCMS (ESI) m/z calcd. for C₃₄H₃₅FN₄O₇S [M+H]⁺ 663.22; found 663.1. The compound structure is confirmed.

To a 25 ml triple-necked vial was sequentially added compound TPD5430-2 (110 mg, 0.166 mmol), tert-butyl 3-acridinecarboxylate hydrochloride (38.58 mg, 0.1992 mmol), potassium carbonate (45.89 mg, 0.332 mmol), sodium iodide (49.8 mg, 0.332 mmol) and N,N-dimethylformamide (5 ml). The reaction was carried out under nitrogen protection at 80° C. for 16 hours. The reaction solution was poured into water (20 ml). It was extracted three times with EA (10 ml). The organic phases were combined, washed with saturated brine, dried over anhydrous sodium sulfate, filtered, and concentrated to dryness. The residue was purified by preparative plate with unfolding agent ratio DCM/MeOH=15/1 to give compound TPD005430-2 (50 mg, yellow solid, purity 96.731%), yield: 40.24%. LCMS (ESI) m/z calcd. for C₄₁H₄₆FN₅O [M+H]⁺ 724.34; found 724.4. ¹H NMR (400 MHz, CDCl₃): δ=9.19 (s, 1H), 8.88 (s, 1H), 8.39 (d, J=5.2 Hz, 1H), 7.60 (d, J=8.8 Hz, 2H), 7.48-7.46 (m, 2H), 7.41 (s, 1H), 7.15 (t, J=8.8 Hz, 3H), 7.05 (t, J=6.4 Hz, 2H), 6.33 (d, J=5.2 Hz, 1H), 3.96 (s, 3H), 3.66-3.57 (m, 2H), 3.56-3.48 (m, 3H), 3.28-3.18 (m, 4H), 2.52-2.48 (m, 2H), 2.29-2.21 (m, 1H), 2.16-2.10 (m, 1H), 1.73-1.68 (m, 5H), 1.54-1.49 (m, 2H), 1.46 (s, 9H). The compound structure is confirmed.

A 50 ml triple vial was charged with compound TPD5488-2 (100 mg, 0.1382 mmol), dichloromethane (6 ml) and trifluoroacetic acid (3 ml). the reaction was carried out at 20° C. for 16 h. The reaction was carried out at 20° C. for 2 h. The reaction was carried out at 20° C. for 2 h. The reaction was completed with the addition of compound TPD5430-3 (90 mg, yellow solid). Concentration to dryness afforded the crude compound TPD5430-3 (90 mg, yellow solid, purity 79.272%), yield: 66.06%. LCMS (ESI) m/z calcd. for C₃₇H₃₈FN₅O₆ [M+H]⁺ 668.28; found 668.3. ¹H NMR (400 MHz, DMSO_d₆): δ=14.78 (s 1H), 10.32 (s, 1H), 10.00 (s, 1H), 8.57 (d, J=6.8 Hz, 1H), 7.84 (d, J=9.2 Hz, 2H), 7.66-7.62 (m, 2H), 7.54 (s, 1H), 7.33 (d, J=9.2 Hz, 2H), 7.16 (t, J=9.2 Hz, 2H), 6.90 (s, 1H), 6.71 (d, J=6.8 Hz, 1H), 4.29-4.07 (m, 7H), 4.00 (s, 3H), 3.38-3.17 (m, 4H), 2.27-2.19 (m, 1H), 2.16-2.10 (m, 1H), 1.70-1.57 (m, 3H), 1.49 (d J=8.4 Hz, 4H). The compound structure is confirmed.

A 25 ml triple-necked vial was sequentially charged with compound TPD5430-3 (45 mg, 0.0674 mmol), N,N-dimethylformamide (3 ml), (2S,4R)-4-hydroxy-N-(4-(4-(4-methylthiazol-5-yl)phenylmethyl)pyrrolidine-2-carboxamide hydrochloride (32.09 mg, 0.1011 mmol), HOBt (13.66 mg, 0.1011 mmol), EDCI (19.38 mg, 0.1011 mmol) and triethylamine (40.84 mg, 0.4044 mmol). The reaction was carried out under nitrogen protection at 25° C. for 16 hours. The reaction solution was decanted into water (10 ml). It was extracted three times with EA (3 ml). The organic phases were combined, washed with saturated brine, dried over anhydrous sodium sulfate, filtered, and concentrated to dryness. The residue was purified by preparative plate with unfolding agent ratio DCM/MeOH=4/1 to give compound TPD005430 (6.65 mg, yellow solid, purity 97.127%), yield: 4.97%. LCMS (ESI) m/z calcd. for C₅₃H₅₅FN₈O₇S [M+H]⁺ 967.39; found 967.7. ¹H NMR (400 MHz, DMSO_d₆): δ=10.19 (s, 1H), 10.08 (s, 1H), 8.98 (s, 1H), 8.80 (br. s., 0.35H), 8.46 (br. s., 0.76H), 8.35 (br. s., 1H), 7.75 (d, J=6.4 Hz, 2H), 7.65 (br. s., 2H), 7.47-7.43 (m, 2H), 7.38 (s, 3H), 7.21-7.14 (m, 4H), 6.94 (s, 1H), 6.27 (br. s., 1H), 5.12 (s, 1H), 4.42-4.25 (m, 4H), 3.90 (s, 3H), 3.56-3.44 (m, 6H), 3.23 (s, 1H), 3.15 (s, 3H), 2.51 (s, 3H), 2.20 (s, 1H), 2.06-1.96 (m, 3H), 1.90-1.85 (m, 1H), 1.55-1.35 (m, 7H), 1.24 (s, 2H). The compound structure is confirmed.

### Example 21: Synthesis of Compound TPD005431

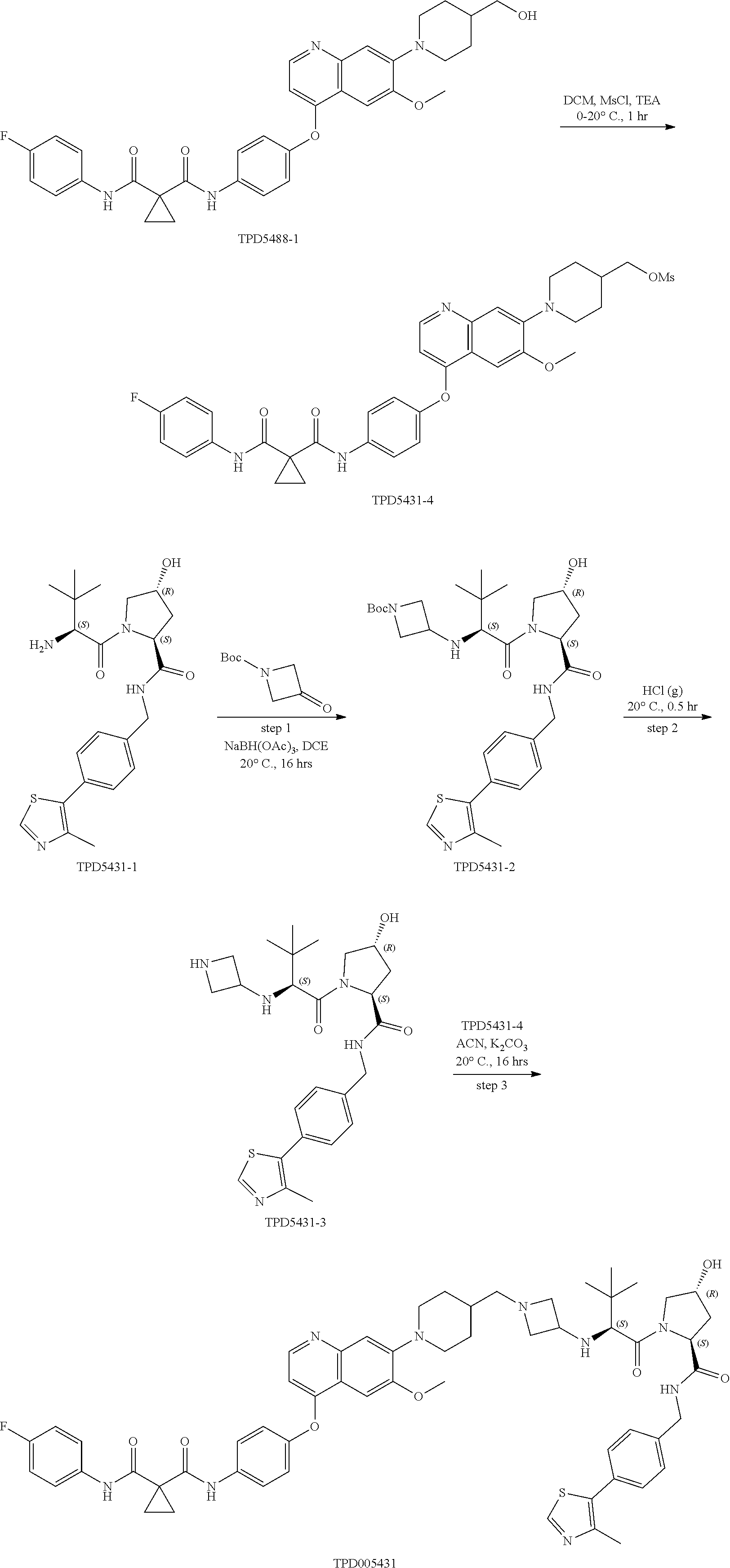

A 25 ml single-necked vial was charged sequentially with tert-butyl 3-oxo-azetidine-1-carboxylate (70 mg, 0.4065 mmol), compound TPD5431-1 (189.85 mg, 0.4065 mmol), sodium triacetoxyborohydride 258.46 mg, 1.2195 mmol), ethylene dichloride (6 ml) and glacial acetic acid (73.23 mg, 1.2195 mmol), the reaction was carried out at 50° C. for 16 hours. After the reaction was completed, the reaction solution and poured into 10 ml of saturated aqueous ammonium chloride solution, extracted three times with DCM (10 ml), the organic phases were combined, washed with saturated brine, dried over anhydrous sodium sulfate, filtered, and the residue was purified by means of a preparative plate, the unfolding agent was DCM/MeOH=10/1, to give the compound TPD5431-2 (210 mg, white solid, purity 89.921%), yield: 79.31%. LCMS (ESI) m/z calcd. for C₃₀H₄₃N₅O₅S [M+H]⁺ 586.3; found 586.1. ¹H NMR (400 MHz, DMSO_d₆): δ=8.99 (d, J=6.2 Hz, 1H), 8.59 (t, J=6.0 Hz, 1H). 7.51-7.31 (m, 4H), 5.07 (d, J=2.8 Hz, 1H), 4.50-4.32 (m, 3H), 4.26-4.20 (m, 1H), 4.12-3.69 (m, 3H), 3.81-3.50 (m, 4H), 3.39 (br. s., 1H), 3.00 (br. s., 1H). 2.45 (d, J=3.2 Hz, 3H), 2.13-1.79 (m, 2H), 1.36 (d, J=3.2 Hz, 9H), 0.92 (s, 9H). The compound structure is confirmed.

A 25 ml single-necked vial was charged with compound TPD5431-2 (180 mg, 0.3073 mmol) and hydrogen chloridemethanol solution (4N, 6 mL). The reaction was carried out at 25° C. for 30 minutes. The reaction was completed and concentrated under reduced pressure to give compound TPD5431-3 (185 mg, yellow solid, purity 85.380%), yield: 98.44%. LCMS (ESI) m/z calcd. for C₂₅H₃₅N₅O₃S [M+H]⁺ 486.3; found 486.1. The compound structure is confirmed.

A 25 ml single-necked vial was charged with compound TPD5488-1 (100 mg, 0.171 mmol), DCM (6 ml), and triethylamine (51.91 mg, 0.513 mmol), and methanesulfonyl chloride (29.38 mg, 0.2565 mmol) was added dropwise after the temperature was lowered to 0° C. The reaction was carried out at 0° C. for 1 h. The reaction was carried out at 0° C. for 1 h. The reaction was carried out at 0° C. for 2 h. The temperature of the reaction was reduced to 0° C. for 1 hour. The reaction was carried out at 0° C. for 1 hour. After the reaction was completed it was poured into water (10 ml) and extracted three times with DCM (3 ml). The combined organic phases were washed with water, brine, dried over saturated sodium sulfate filtered and concentrated to dryness to give the crude compound TPD5431 (50 mg, yellow oil, purity 40.853%), yield: 25.26%. LCMS (ESI) m/z calcd. for C₃₄H₃₅FN₄O₇S [M+H]⁺ 663.2; found 229.2 and 663.1. The compound structure is confirmed.

To a 25 mL single-necked vial was sequentially added compound TPD5431-4 (70 mg, 0.1056 mmol), compound TPD5431-3 (102.57 mg, 0.2112 mmol), potassium carbonate (58.38 mg, 0.4224 mmol), sodium iodide (31.68 mg, 0.2112 mmol) and DMF (5 ml). The reaction was carried out at 80° C. for 16 hours. After the reaction was completed it was cooled to room temperature, decanted into water (10 ml) and extracted three times with EA (5 ml). The organic phases were combined, washed with saturated brine, dried over anhydrous sodium sulfate, filtered, and concentrated to dryness. The residue was prepared by high performance liquid chromatography (HPLC) with the following parameters: column: XBridge-1 5 μm 19-150 mm; mobile phase: acetonitrile-water (0.1% ammonia); gradient: 30-90/8 min to afford the compound TPD005431 (11.2 mg, off-white solid, purity 95.035%) in a yield of 9.56%. LCMS (ESI) m/z calcd. for C₅₈H₆₆FN₉O₇S [M/2+H]⁺526.7; found 526.9. ¹H NMR (400 MHz, DMSO_d₆): δ=10.17 (s, 1H), 10.06 (s, 1H), 8.97 (d, J=6.0 Hz, 1H), 8.59 (t, J=5.9 Hz, 1H). 8.34 (d, J=5.1 Hz, 1H), 7.74 (d, J=8.8 Hz, 2H), 7.66-7.62 (m, 2H), 7.48-7.32 (m, 5H), 7.23-7.09 (m, 4H), 6.93 (s, 1H), 6.26 (d, J=5.2 Hz, 1H), 5.14 (d, J=10.2 Hz, 1H), 4.50-4.29 (m, 3H), 4.25-4.20 (m, 1H), 3.90 (s, 3H), 3.64 (d, J=11.1 Hz, 1H), 3.58-3.41 (m, 6H), 3.22-3.10 (m, 3H), 3.00 (br. s., 1H), 2.62-2.56 (m 1H), 2.44 (s, 3H), 2.39-2.32 (m, 2H), 2.20-2.14 (m, 1H), 2.10-1.97 (m, 3H), 1.93-1.85 (m, 1H), 1.55-1.43 (m, 4H), 1.42-1.33 (m, 2H), 1.23 (s, 1H), 0.96-0.79 (m, 9H). The compound structure is confirmed.

### Example 22: Synthesis of Compound TPD005432

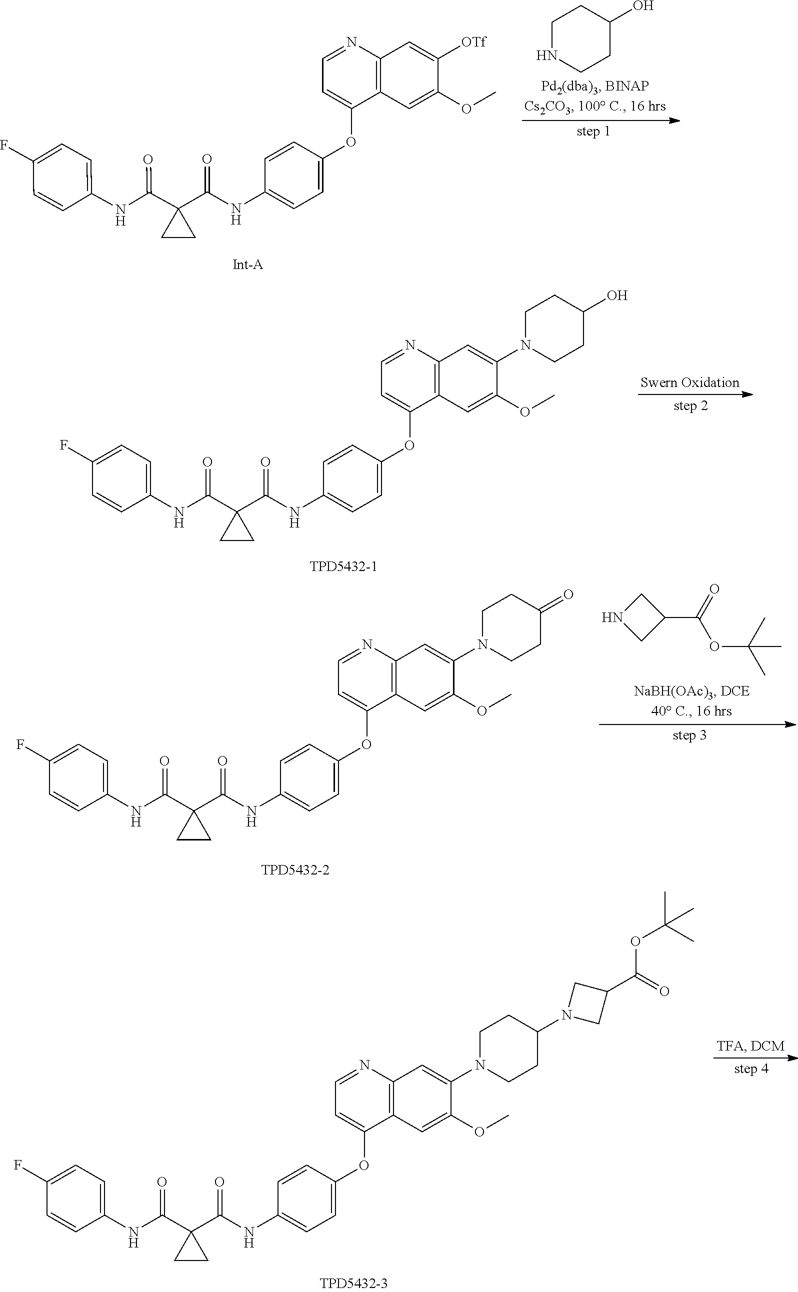 [SMILES: COc1cc2c(Oc3ccc(NC(=O)C4(C(=O)Nc5ccc(F)cc5)CC4)cc3)ccnc2cc1OS(=O)(=O)C(F)(F)F ; OC1CCNCC1 ; COc1cc2c(Oc3ccc(NC(=O)C4(C(=O)Nc5ccc(F)cc5)CC4)cc3)ccnc2cc1N1CCC(O)CC1 ; COc1cc2c(Oc3ccc(NC(=O)C4(C(=O)Nc5ccc(F)cc5)CC4)cc3)ccnc2cc1N1CCC(=O)CC1 ; CC(C)(C)OC(=O)C1CNC1 ; COc1cc2c(Oc3ccc(NC(=O)C4(C(=O)Nc5ccc(F)cc5)CC4)cc3)ccnc2cc1N1CCC(N2CC(C(=O)OC(C)(C)C)C2)CC1]

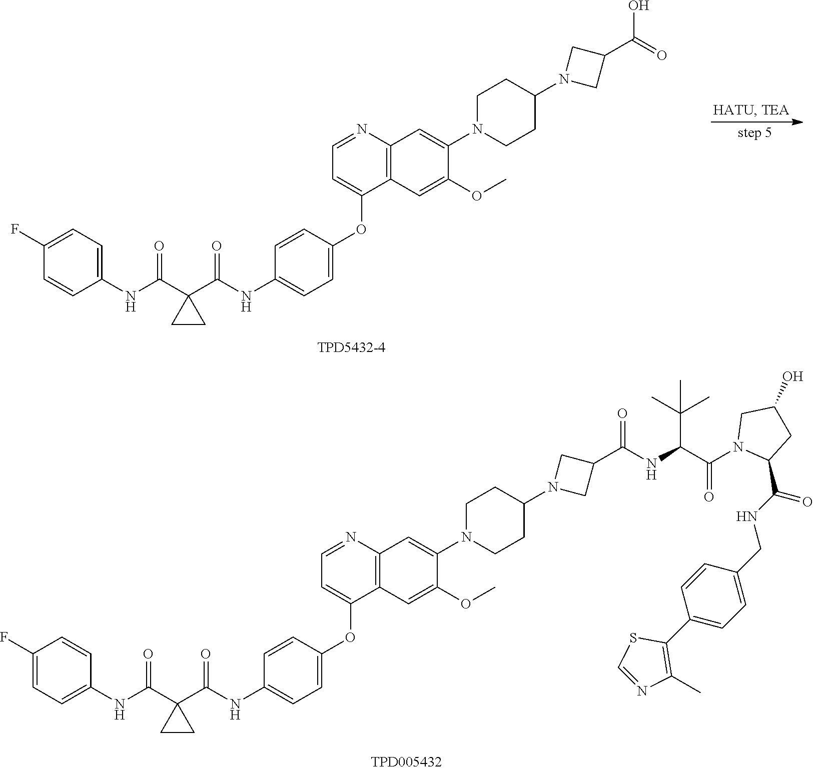 [SMILES: COc1cc2c(Oc3ccc(NC(=O)C4(C(=O)Nc5ccc(F)cc5)CC4)cc3)ccnc2cc1N1CCC(N2CC(C(=O)O)C2)CC1 ; COc1cc2c(Oc3ccc(NC(=O)C4(C(=O)Nc5ccc(F)cc5)CC4)cc3)ccnc2cc1N1CCC(N2CC(C(=O)N[C@H](C(=O)N3C[C@H](O)C[C@H]3C(=O)NCc3ccc(-c4scnc4C)cc3)C(C)(C)C)C2)CC1]

To a 100 ml triple-necked vial were sequentially added compound int-A (1400 mg, 2.2579 mmol), 4-hydroxypiperidine (502.84 mg, 4.97134 mmol), Pd₂ (dba)₃ (413.85 mg, 0.45194 mmol), BINAP (562.82 mg, 0.9038 mmol), cesium carbonate (1472.51 mg, 4.5194 mmol) and dioxane (50 ml). The reaction was carried out under nitrogen protection at 100° C. for 16 hours. After completion of the reaction, the reaction solution was reduced to room temperature, water was added to the reaction solution and extracted three times with EtOAc (100 ml). The combined organic layers were washed with brine (200 ml), dried over anhydrous sodium sulfate, filtered and evaporated to give a residue. The residue was purified by silica gel column chromatography with eluent and ratio of DCM/MeOH=100/1˜20/1 to give the product compound TPD5432-1 (595 mg, yellow solid, purity 85.651%), yield: 37.67%. LCMS (ESI) m/z calcd. for C₃₂H₃₁FN₄O₅ [M+H]⁺ 571.23. found 571.2. The compound structure is confirmed.

Oxalyl chloride (143.48 mg, 1.1304 mmol) and DCM (2 ml) were added to a 50 ml three-necked vial under nitrogen protection. Cooling down to −78° C. a solution of DMSO (176.34 mg, 2.2608 mmol) in DCM (1 ml) was added dropwise. The resulting mixture was kept at −78° C. for 0.5 hours. Then a DCM (2 ml) solution of compound TPD5432-1 (430 mg, 0.7536 mmol) was added dropwise at −78° C. The resulting mixture was kept at −78° C. for 1 hour. Then TEA (228.77 mg, 2.2608 mmol) was added dropwise at −78° C. The resulting mixture was naturally warmed up for the reaction to 20° C. and kept for 0.5 hours. After completion of the reaction, the reaction was quenched by addition of saturated aqueous sodium bicarbonate. The mixture was extracted with DCM (20 ml×3). The combined organic layers were washed with brine (20 ml), dried over anhydrous sodium sulfate, filtered and evaporated to give a residue. Purification of the residue by preparative TLC (DCM/MeOH=10/1) afforded the product compound TPD5432-2 (290 mg, yellow solid, purity 92.290%), yield: 62.46%. LCMS (ESI) m/z calcd. for C₃₂H₂₉FN₄O₅ [M+H]⁺ 569.21; found 569.0. ¹H NMR (400 MHz, CDCl₃): δ=9.35 (s, 1H), 8.66 (s, 1H), 8.49 (d, J=5.2 Hz, 1H), 7.58-7.46 (m, 5H), 7.19-7.17 (m, 2H), 7.08-7.04 (m, 2H), 6.46 (d, J=5.2 Hz, 1H). 4.07 (s, 3H), 3.57-3.54 (m, 4H), 2.72-2.69 (m, 4H), 1.75-1.65 (m, 4H). The compound structure is confirmed.

A 50 ml single-necked vial was charged with DCE (10 ml), compound TPD5432-2 (150 mg, 0.2638 mmol), tert-butyl 3-acridinecarboxylate hydrochloride (255.45 mg, 1.319 mmol), sodium triacetoxyborohydride (167.73 mg, 0.7913 mmol), tetraisopropyl titanate (0.5 ml). The reaction solution was heated up to 40° C. under nitrogen protection and then reacted for 16 hours. After the reaction was completed, water was added to the mixture and the mixture was extracted three times with EtOAc (10 ml). The combined organic layers were washed with brine (20 ml), dried over anhydrous sodium sulfate, filtered and evaporated to give the residue. Purification of the residue by preparative TLC (DCM/MeOH=10/1) afforded the product compound TPD5432-3 (100 mg, yellow solid, purity 96.741%), yield: 51.67%. LCMS (ESI) m/z calcd. for C₄₀H₄₄ FN₅O₆ [M+H]⁺ 710.33; found 710.5. The compound structure is confirmed.

A 50 ml single-necked vial was charged with compound TPD5432-3 (50 mg, 0.0704 mmol), DCM (2 ml), trifluoroacetic acid (2 ml). The resulting mixture was stirred at 20° C. for 2 hours. The reaction was completed and the mixture was spun dry to obtain the crude product compound TPD5432-4 (46 mg, white solid, purity 92.176%), yield: 92.19%. LCMS (ESI) m/z calcd. for C₃₆H₃₆ FN₅O₆ [M+H]⁺ 654.26; found 654.3. The compound structure is confirmed.

Compound TPD5432-4 (46 mg, 0.0704 mmol), HATU (40.15 mg, 0.1056 mmol), (2S,4R)-1—((S)-2-amino-3,3-dimethylbutanoyl)-4-hydroxy-N-(4-(4-methylthiazol-5-yl)benzyl)pyrrolidine-2-formamide hydrochloride (40.3 mg, 0.0704 mmol) and TEA (35.62 mg, 0.352 mmol). The reaction was carried out under nitrogen protection at 15° C. for 16 hours. After completion of the reaction, the reaction solution was quenched with water. It was extracted three times with ethyl acetate (10 ml). The combined organic layers were washed with brine (20 ml), dried over anhydrous sodium sulfate, filtered and evaporated to give the residue. The crude product was purified by preparative HPLC (column: sunfire, 5 m, 19-150 mm; detector: 254 nm; mobile phase: ACN/H₂O (0.1% FA in H₂O); gradient: 10-50/8 min; retention time: 7.0 min) to give the product compound TPD005432 (23.5 mg, yellow solid, purity 97.684%), yield: 22.03%. LCMS (ESI) m/z calcd. for C₅₈H₆₄FN₉O₈S [M+H]⁺ 1066.46; found 533.8 (half peak); 356.2 (one-third peak)¹H NMR (400 MHz, DMSO_d₆): δ=10.18 (s, 1H), 10.06 (s, 1H), 8.98 (s, 1H), 8.59 (t, J=6.0 Hz, 1H), 8.43 (d, J=5.2 Hz, 1H), 8.18 (s, 1H), 8.05 (d, J=9.2 Hz, 1H), 7.76 (d, J=8.9 Hz, 2H), 7.64 (dd, J=9.0, 5.1 Hz, 2H), 7.47-7.37 (m, 5H), 7.31 (s, 1H), 7.23-7.13 (m, 4H), 6.39 (d, J=5.2 Hz, 1H), 4.57 (d, J=9.4 Hz, 1H), 4.48-4.40 (m, 2H), 4.37 (s, 1H), 4.22 (dd, J=15.7, 5.3 Hz, 1H). 3.94 (s, 3H), 3.67-3.64 (m, 2H), 3.44-3.37 (m, 2H), 3.22 (dd, J=28.7, 6.4 Hz, 3H), 2.74 (t, J=10.0 Hz, 2H), 2.45 (s, 3H), 2.30 (br. s., 1H), 2.15-1.75 (m, 5H). 1.48 (s, 4H), 1.36 (d, J=9.0 Hz, 2H), 1.23 (s, 2H), 0.95-0.93 (m, 9H). The compound structure is confirmed.

### Example 23: Synthesis of Compound TPD005436

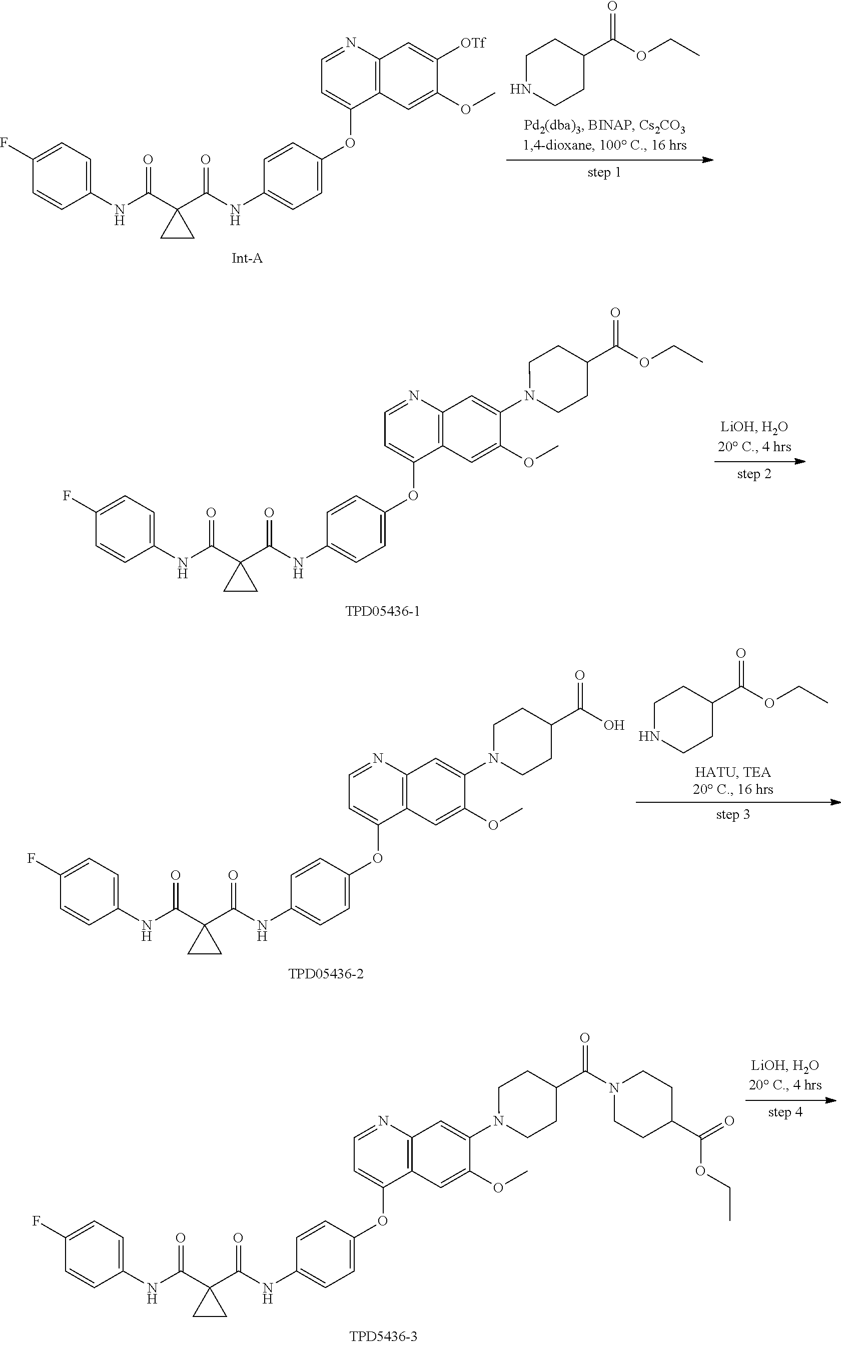

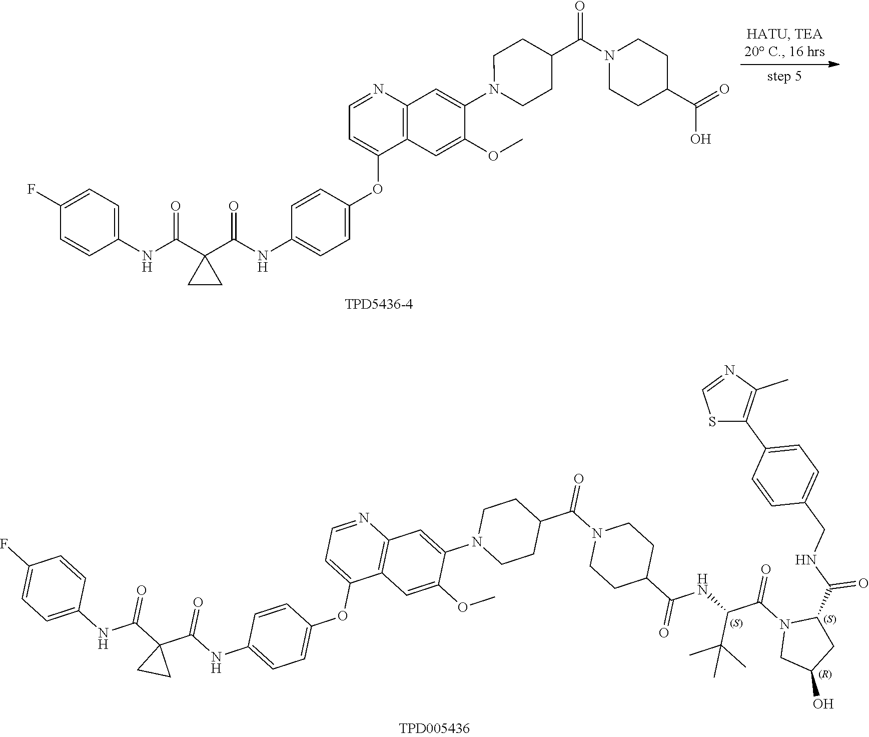

To a 250 ml triple-necked vial were sequentially added compound int-A (1.4 g, 2.2597 mmol), ethyl 4-piperidine carboxylate (710.49 mg, 4.5194 mmol), cesium carbonate (1.472 g, 4.5194 mmol), BINAP (562.82 mg, 0.9038 mmol), Pd₂ (dba)₃ (413.85 mg, 0.4519) and 1,4-dioxane (70 ml). The reaction was carried out under nitrogen protection at 100° C. for 16 hours. After the reaction was completed, the reaction solution was reduced to room temperature and concentrated to dryness, and the residue was purified by silica gel column with eluent ratio of DCM/MeOH=100/1˜30/1 to afford the compound TPD5436-1 (660 mg, brown solid, purity 72.545%), yield: 33.81%. LCMS (ESI) m/z calcd. for C₃₅H₃₅FN₄O₆ [M+H]⁺ 627.3; found 627.4. The compound structure is confirmed.

To a 50 ml single-necked vial was sequentially added compound TPD5436-1 (600 mg, 0.9574 mmol), lithium hydroxide monohydrate (80.35 mg, 1.9148 mmol) and methanol/water=2:1 (10 ml). The reaction was carried out at 20° C. for 4 h. After completion of the reaction, it was concentrated to dryness to give the crude compound TPD5436-2 (700 mg, brown solid), yield: 86.17%. LCMS (ESI) m/z calcd. for C₃₃H₃₁FN₄O₆ [M+H]⁺ 599.2; found 599.1. The compound structure is confirmed.

To a 25 ml single-necked vial were sequentially added compounds TPD5436-2 (200 mg, 0.3341 mmol), ethyl 4-piperidinecarboxylate (63.03 mg, 0.4009 mmol), HATU (254.07 mg, 0.6682 mmol), triethylamine (101.43 mg, 0.6682 mmol) and DMF (5 ml). The reaction was carried out under nitrogen protection at 20° C. for 16 hours. After the reaction was completed, the reaction solution was poured into water (6 ml) and extracted three times with EA (3 ml). The organic phases were combined, washed with saturated brine, dried over anhydrous sodium sulfate, filtered, and concentrated to dryness. The residue was purified by preparative plate with unfolding agent ratio of DCM/MeOH=10/1 to afford the compound TPD5436-3 (200 mg, brown solid, purity 80.484%), yield: 65.31%. LCMS (ESI) m/z calcd. for C₄₁H₄₄FN₅O₇ [M+H]⁺ 738.3; found 738.2; ¹H NMR (400 MHz, DMSO_d₆): δ=10.19 (s, 1H), 10.06 (s, 1H), 8.44 (d, J=5.2 Hz, 1H), 7.76 (d, J=8.9 Hz, 2H), 7.66-7.63 (m, 2H), 7.47 (s, 1H), 7.31 (s, 1H), 7.21 (d, J=9.0 Hz, 2H), 7.18-7.13 (m, 2H), 6.40 (d, J=5.2 Hz, 1H), 4.11-4.05 (m, 2H), 3.95 (s, 3H), 3.62 (d, J=11.1 Hz, 2H), 3.24-3.06 (m, 2H), 2.92-2.83 (m, 3H), 2.79-2.68 (m, 4H), 2.65-2.59 (m, 1H), 1.89-1.70 (m, 6H), 1.47 (s, 4H), 1.21-1.17 (m, 3H). The compound structure is confirmed.

To a 50 ml single-necked vial was sequentially added compound TPD5436-3 (200 mg, 0.2711 mmol), lithium hydroxide monohydrate (22.75 mg, 0.5422 mmol) and methanol/water=2:1 (3 ml). The reaction was carried out at 20° C. for 4 h. After completion of the reaction, it was concentrated to dryness to give the crude compound TPD5436-4 (250 mg, brown solid), yield: 85.58%. LCMS (ESI) m/z calcd. for C₃₉H₄₀FN₅O₇ [M+H]⁺ 710.3; found 710.2. The compound structure is confirmed.

A 25 ml single-necked vial was sequentially charged with compounds TPD5436-4 (250 mg, 0.3522 mmol), (2S,4R)-1-((S)-2-amino-3,3-dimethylbutanoyl)-4-hydroxy-N-(4-(4-methylthiazol-5-yl)benzyl)pyrrolidine-2-carboxamide hydrochloride (197.38 mg, 0.4226 mmol), HATU (267.83 mg, 0.7044 mmol), triethylamine (71.28 mg, 0.7044 mmol) and DMF (5 ml). The reaction was carried out under nitrogen protection at 20° C. for 16 hours. After the reaction was completed, the reaction solution was poured into water (6 ml) and extracted three times with EA (3 ml). The organic phases were combined, washed with saturated brine, dried over anhydrous sodium sulfate, filtered, and concentrated to dryness. The residue was purified by preparative plate with unfolding agent ratio DCM/MeOH=10/1 to afford compound TPD005436 (31.2 mg, light yellow solid, purity 96.544%), yield: 7.47%. LCMS (ESI) m/z calcd. for C₆₁H₆₈FN₉O₉S [M+H]⁺ 1122.5; found 1122.3; ¹H NMR (400 MHz, DMSO_d₆): δ=10.18 (s, 1H), 10.06 (s, 1H), 8.99 (s, 1H), 8.58 ((br. s., 1H), 8.44 (d, J=5.2 Hz, 1H), 7.91 (d, J=8.6 Hz, 1H), 7.76 (d, J=8.9 Hz, 2H). 7.66-7.63 (m, 2H), 7.47 (s, 1H), 7.44-7.38 (m, 4H), 7.31 (s, 1H), 7.21 (d, J=8.9 Hz, 2H), 7.18-7.13 (m, 2H), 6.40 (d, J=5.2 Hz, 1H), 5.14 (d, J=3.3 Hz, 1H). 4.54 (d, J=9.7 Hz, 1H), 4.45-4.31 (m, 3H), 4.35 (s, 1H), 4.25-4.20 (m, 1H), 4.03 (br. s., 1H), 3.95 (s, 3H), 3.68-3.61 (m, 4H), 3.07-3.02 (m, 1H), 2.83-2.67 (m, 4H), 2.45 (s, 3H), 2.08-1.87 (m, 3H), 1.81-1.62 (m, 6H), 1.47 (s, 5H), 1.23 (s, 1H), 0.95-0.92 (m, 9H). The compound structure is confirmed.

### Example 24: Synthesis of compound TPD005452

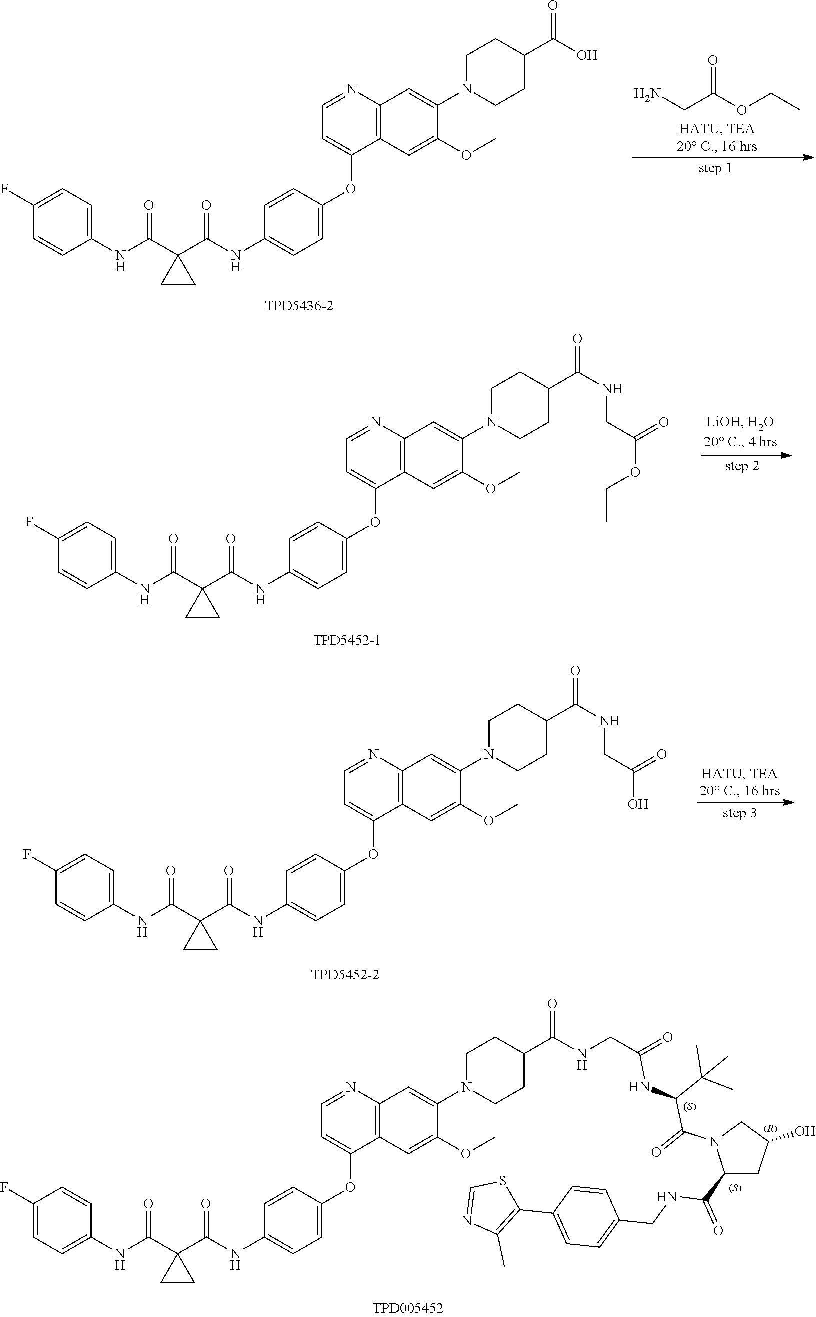

A 25 ml single-necked vial was sequentially charged with the compounds TPD5436-2 (300 mg, 0.5012 mmol), glycine ethyl ester hydrochloride (83.95 mg, 0.6014 mmol), HATU (381.14 mg, 1.0024 mmol), triethylamine (101.43 mg, 1.0024 mmol), and DMF (5 ml). The reaction was carried out under nitrogen protection at 20° C. for 16 hours. After completion of the reaction, the reaction solution was poured into water (6 ml) and extracted three times with EA (3 ml). The organic phases were combined, washed with saturated brine, dried over anhydrous sodium sulfate, filtered, and concentrated to dryness. The residue was purified by preparative plate with the unfolding agent ratio of DCM/MeOH=10/1 to afford the compound TPD5452-1 (190 mg, brown solid, purity 90.489%), yield: 50.18%. LCMS (ESI) m/z calcd. for C₃₇H₃₈FN₅O₇ [M+H]⁺ 684.3; found 684.3; ¹H NMR (400 MHz, DMSO_d₆): δ=10.19 (s, 1H), 10.07 (s, 1H), 8.45 (d, J=5.2 Hz, 1H), 8.32 (t, J=5.9 Hz, 1H), 7.76 (d, J=8.9 Hz, 1H), 7.67-7.63 (m, 2H), 7.48 (s, 1H), 7.34 (s, 1H), 7.23-7.21 (m, 2H), 7.18-7.14 (m, 2H), 6.40 (d, J=5.2 Hz, 1H), 4.13-4.08 (m, 2H), 3.96 (s, 1H), 3.83 (d, J=5.9 Hz, 1H), 3.62 (d, J=11.5 Hz, 1H), 2.74-2.68 (m, 3H), 1.84-1.79 (m, 4H), 1.48 (s, 4H), 1.21 (t, J=7.1 Hz, 1H). The compound structure is confirmed.

To a 50 ml single-necked vial was sequentially added compound TPD5436-3 (190 mg, 0.2779 mmol), lithium hydroxide monohydrate (23.32 mg, 0.5558 mmol) and methanol/water=2:1 (3 ml). The reaction was carried out at 20° C. for 4 h. After completion of the reaction, it was concentrated to dryness to give the crude compound TPD5436-4 (200 mg, brown solid), yield: 76.90%. LCMS (ESI) m/z calcd. for C₃₅H₃₄FN₅O₇ [M+H]⁺ 656.2; found 656.2. The compound structure is confirmed.

A 25 ml single-necked vial was sequentially charged with compound TPD5452-2 (100 mg, 0.1525 mmol), (2S,4R)-1-((S)-2-amino-3,3-dimethylbutanoyl)-4-hydroxy-N-(4-(4-methylthiazol-5-yl)benzyl)pyrrolidine-2-carboxamide hydrochloride (85.46 mg, 0.183 mmol), HATU (115.97 mg, 0.305 mmol), triethylamine (30.86 mg, 0.305 mmol) and DMF (5 ml). The reaction was carried out under nitrogen protection at 20° C. for 16 hours. After completion of the reaction, the reaction solution was poured into water (6 ml) and extracted three times with EA (3 ml). The organic phases were combined, washed with saturated brine, dried over anhydrous sodium sulfate, filtered, and concentrated to dryness. The residue was purified by preparative plate with unfolding agent ratio DCM/MeOH=6/1 to afford compound TPD005452 (28.2 mg, light yellow solid, purity 95.282%), yield: 16.52%; LCMS (ESI) m/z calcd. for C₅₇H₆₂FN₉O₉S [M+H]⁺ 1068.4; found 534.7; ¹H NMR (400 MHz, DMSO_d₆): δ=10.18 (s, 1H), 10.06 (s, 1H), 8.94 (s, 1H), 8.67 (t, J=6.4 Hz, 1H), 8.44 (d, J=5.2 Hz, 1H), 8.23 (t, J=5.6 Hz, 1H), 7.76 (d, J=8.9 Hz, 1H), 7.70-7.63 (m, 3H), 7.46 (s, 1H), 7.44-7.37 (m, 4H), 7.33 (s, 1H), 7.23-7.21 (m, 2H), 7.18-7.13 (m, 2H), 6.40 (d, J=5.2 Hz, 1H), 5.17 (d, J=3.5 Hz, 1H), 4.54 (d, J=9.5 Hz, 1H), 4.48-4.46 (m, 1H), 4.44-4.41 (m, 1H), 4.36 (br. s., 1H), 4.25-4.19 (m, 1H), 3.93 (s, 3H), 3.78 (d, J=6.0 Hz, 1H), 3.69-3.63 (m, 4H), 2.67 (br. s., 2H), 2.43 (s, 3H), 2.01-1.84 (m, 6H), 1.47 (s, 4H), 1.23 (s, 1H), 0.94-0.92 (m, 9H). The compound structure is confirmed.

### Example 25: Synthesis of Compound TPD005453

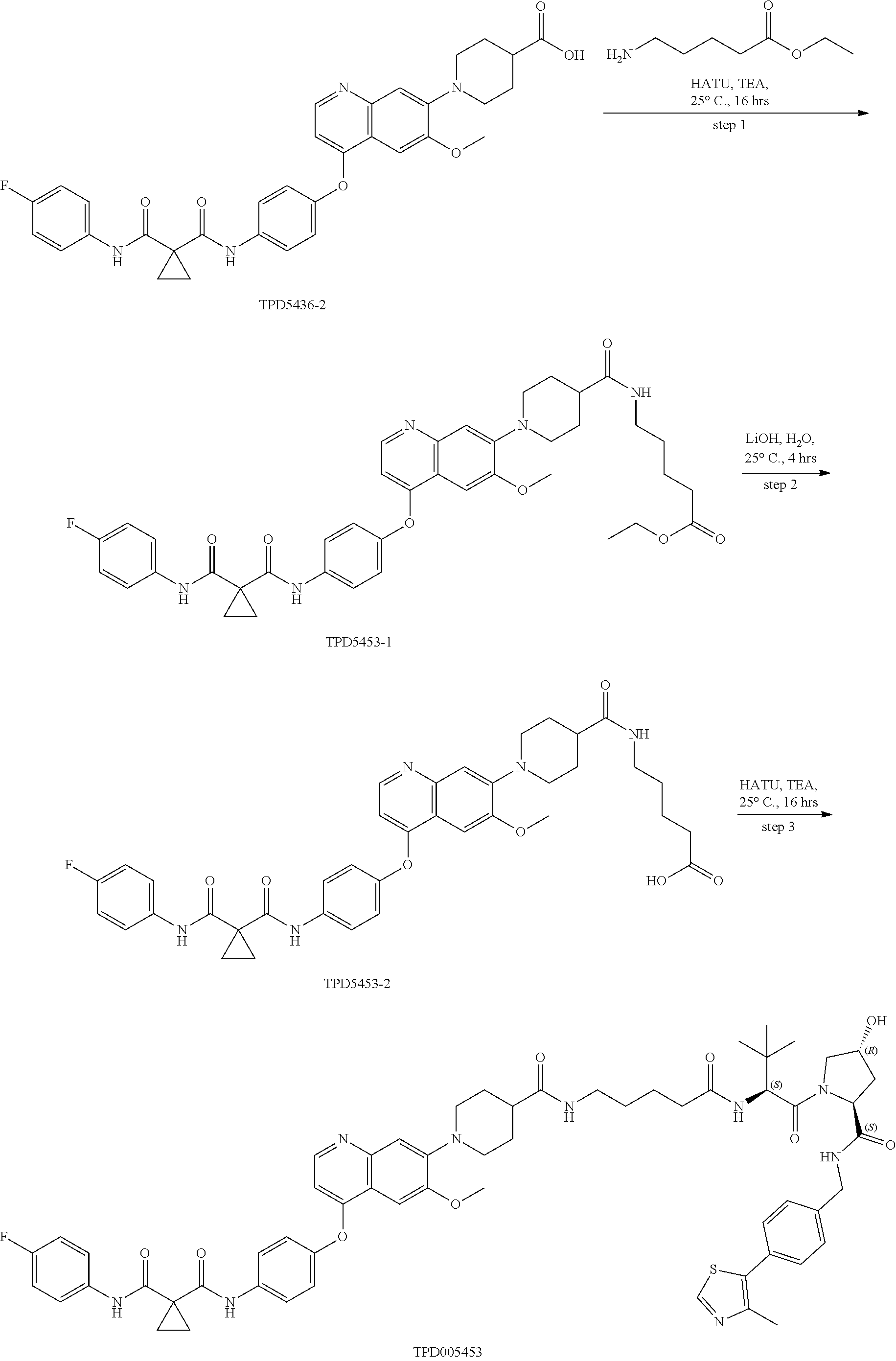

To a 25 ml single-necked vial was sequentially added compound TPD5436-2 (300 mg, 0.5012 mmol), ethyl 5-aminopentanoate hydrochloride (109.26 mg, 0.6014 mmol), HATU (381.14 mg, 1.0024 mmol), triethylamine (101.43 mg, 1.0024 mmol), and DMF (5 ml). The reaction was carried out under nitrogen protection at 25° C. for 16 hours. After completion of the reaction, the reaction solution was poured into water (6 ml) and extracted three times with EA (3 ml). The organic phases were combined, washed with saturated brine, dried over anhydrous sodium sulfate, filtered, and concentrated to dryness. The residue was purified by preparative plate with unfolding agent ratio DCM/MeOH=10/1 to afford compound TPD5453-1 (110 mg, brown solid, purity 82.845%), yield: 25.06%. LCMS (ESI) m/z calcd. for C₄₀H₄₄FN₅O₇ [M+H]⁺ 726.32; found 726.2; ¹H NMR (400 MHz, DMSO_d₆): δ=10.18 (s, 1H), 10.06 (s, 1H), 8.44 (d, J=5.2 Hz, 1H), 7.84 (t, J=5.5 Hz, 1H), 7.76 (d, J=8.8 Hz, 2H), 7.66-7.62 (m, 2H), 7.47 (s, 1H), 7.33 (s, 1H), 7.23-7.20 (m, 2H), 7.18-7.13 (m, 2H), 6.40 (d, J=5.1 Hz, 1H), 4.08-4.03 (m, 2H), 3.95 (s, 3H), 3.62 (d, J=11.1 Hz, 2H), 3.09-3.04 (m, 2H), 2.89 (s, 4H), 2.70-2.63 (m, 2H), 2.30 (s, 1H), 1.79 (br. s., 4H), 1.47 (s, 4H), 1.43-1.38 (m, 2H), 1.18 (t, J=7.1 Hz, 3H). The compound structure is confirmed.

To a 50 ml single-necked vial was sequentially added compound TPD5453-1 (110 mg, 0.1516 mmol), lithium hydroxide monohydrate (12.72 mg, 0.3032 mmol) and methanol/water=2/1 (3 ml). The reaction was carried out at 25° C. for 4 h. After completion of the reaction, it was concentrated to dryness to give the crude compound TPD5453-2 (110 mg, brown solid), yield: 90.50%. LCMS (ESI) m/z calcd. for C₃₈H₄₀FN₅O₇ [M+H]⁺ 698.3; found 698.2. The compound structure is confirmed.

A 25 ml single-necked vial was sequentially charged with compound TPD5453-2 (100 mg, 0.1433 mmol), (2S,4R)-1-((S)-2-amino-3,3-dimethylbutanoyl)-4-hydroxy-N-(4-(4-methylthiazol-5-yl)benzyl)pyrrolidine-2-carboxamide hydrochloride (80.31 mg, 0.17196 mmol), HATU (108.97 g, 0.2866 mmol), triethylamine (29 mg, 0.2866 mmol) and DMF (3 ml). The reaction was carried out under nitrogen protection at 25° C. for 16 hours. After completion of the reaction, the reaction solution was poured into water (6 ml) and extracted three times with EA (3 ml). The organic phases were combined, washed with saturated brine, dried over anhydrous sodium sulfate, filtered, and concentrated to dryness. The residue was purified by preparative plate with unfolding agent ratio DCM/MeOH=8/1 to give compound TPD005453 (21.8 mg, light yellow solid, purity 97.007%), yield: 13.26%. LCMS (ESI) m/z calcd. for C₆₀H₆₈FN₉O₉S [M+H]⁺ 1110.5; found 555.8 (half peak); ¹H NMR (400 MHz, DMSO_d₆): δ=10.19 (s, 1H), 10.07 (s, 1H), 8.98 (s, 1H), 8.59 (t, J=6.4 Hz, 1H), 8.44 (d, J=5.2 Hz, 1H), 7.89 (d, J=9.4 Hz, 1H), 7.83 (t, J=5.4 Hz 1H), 7.76 (d, J=8.9 Hz, 2H), 7.66-7.63 (m, 2H), 7.47 (s, 1H), 7.43-7.37 (m, 4H), 7.32 (s, 1H), 7.22 (d, J=8.9 Hz, 2H), 7.18-7.13 (m, 2H), 6.40 (d, J=5.1 Hz, 1H), 5.16 (d, J=3.4 Hz, 1H), 4.55 (d, J=9.4 Hz, 1H), 4.47-4.41 (m, 2H), 4.36 (br. s., 1H), 4.24-4.19 (m, 1H), 3.95 (s, 3H), 3.66-3.60 (m, 4H), 3.06-3.04 (m, 2H). 2.67-2.66 (m, 2H), 2.44 (s, 3H), 2.31-2.24 (m, 2H), 2.16-2.10 (m, 1H), 2.08-1.99 (m, 2H), 1.94-1.87 (m, 1H), 1.79 (br. s., 4H), 1.47 (s, 4H), 1.42-1.35 (m. 2H), 1.23 (s, 1H), 0.94 (s, 9H). The compound structure is confirmed.

### Example 26: Synthesis of Compound TPD005457

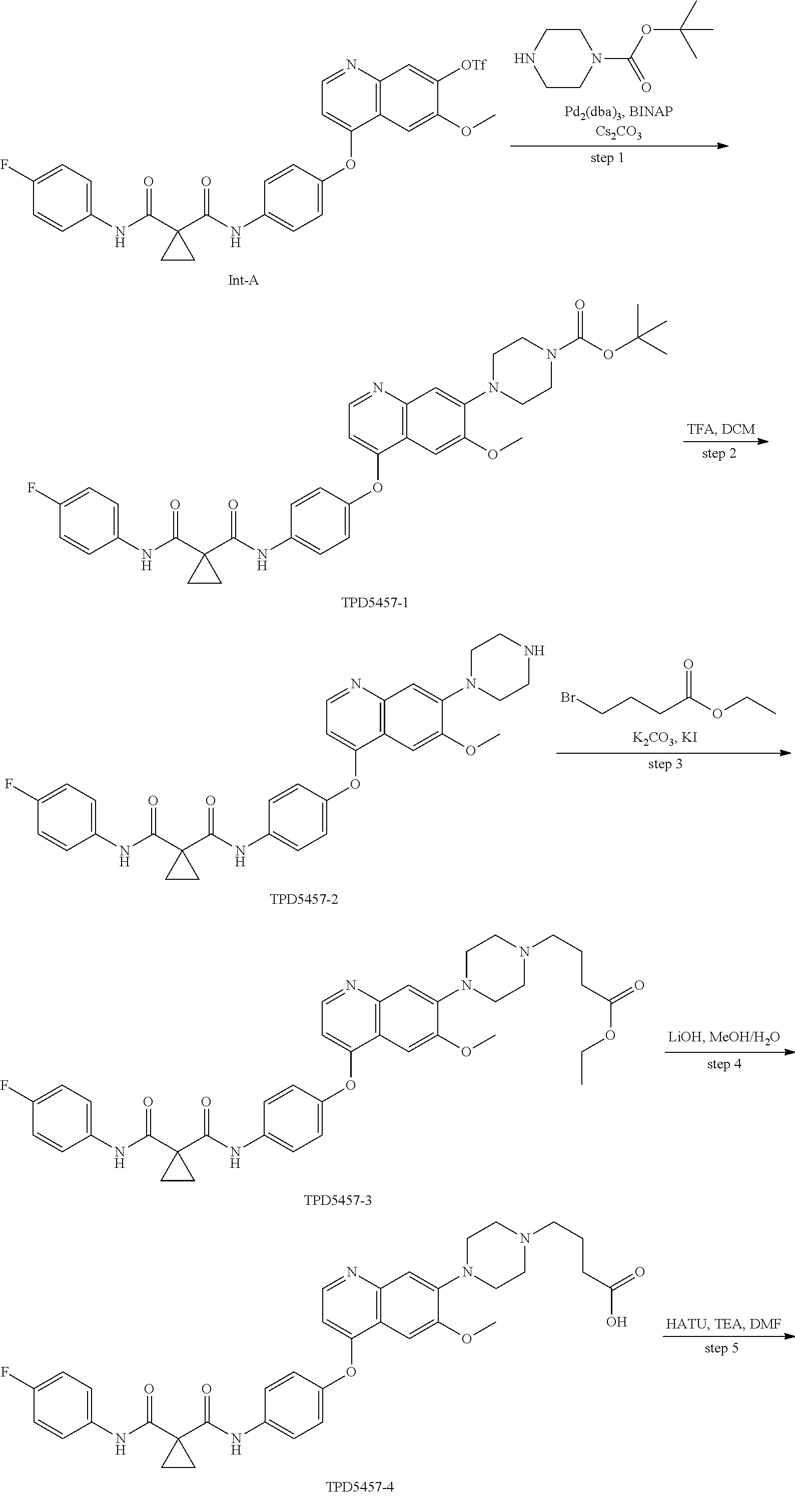

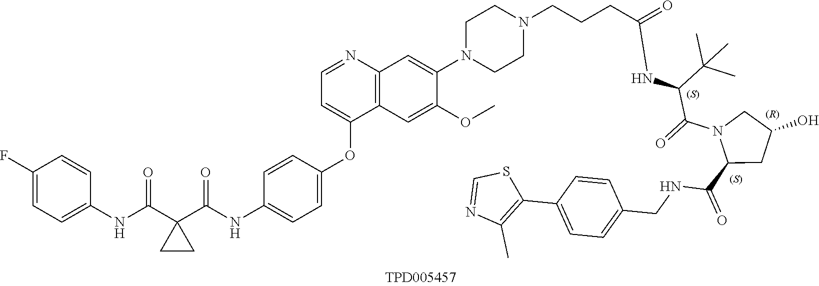

To a 25 ml single-necked vial was sequentially added compound int-A (200 mg, 0.3228 mmol), tert-butyl piperazine-1-carboxylate (132.27 mg, 0.7101 mmol), cesium carbonate (210.35 mg, 0.6456 mmol), dioxane (10 ml), Pd₂ (dba)₃ (59.12 mg, 0.0645 mmol) and 1,1′-binaphthyl-2,2′-bisdiphenylphosphine (80.4 mg, 0.1291 mmol). The reaction was protected by nitrogen at 100° C. for 16 hours. After the reaction was completed, the reaction solution was reduced to room temperature and concentrated to dryness, and the residue was purified by silica gel chromatographic column with eluent ratio of DCM/MeOH=100/1˜50/1 to obtain the compound TPD5457-1 (63.33 mg, yellow solid, purity 88.841%), yield: 53.16%. LCMS (ESI) m/z calcd. for C₃₆H₃₈FN₅O₆ [M+H]⁺ 656.3; found 656.3; ¹H NMR (400 MHz, DMSO_d₆): δ=10.19 (s, 1H), 10.06 (s, 1H), 8.45 (d, J=5.2 Hz, 1H), 7.76 (d, J=8.9 Hz, 2H), 7.67-7.62 (m, 2H). 7.50 (s, 1H), 7.34 (s, 1H), 7.24-7.12 (m, 4H), 6.41 (d, J=5.2 Hz, 1H), 3.96 (s, 3H), 3.53 (br. s., 4H), 3.18-3.01 (m, 4H), 1.49-1.37 (m, 13H). It showed that the structure of the compound is correct.

A 25 ml single mouth vial was charged with compound TPD5457-1 (280 mg, 0.4270 mmol), DCM (2 ml) and TFA (2 ml). the reaction was carried out for 30 min at 25° C. The reaction solution was concentrated to dryness to give crude compound TPD5457-2 (440 mg, white solid) in 94.24% yield. The reaction solution was concentrated to dryness to give the crude compound TPD5457-2 (440 mg, white solid, purity 82.094%) in 94.24% yield. LCMS (ESI) m/z calcd. for C₃₁H₃₀FN₅O₄ [M+H]⁺ 556.2; found 556.1. The compound structure is confirmed.

A 100 ml single-necked vial was charged with compound TPD5457-2 (300 mg, 0.54 mmol), ethyl 4-bromobutyrate (210.66 mg, 1.08 mmol), potassium carbonate (223.9 mg, 1.62 mmol), potassium iodide (8.96 mg, 0.054 mmol), and acetonitrile (30 mL). the reaction was carried out for 16 h at 20° C. The reaction solution was extracted with EA (30 ml) for 3 times and extracted with EA (30 ml) for 3 times. The reaction solution was poured into water (50 ml) and extracted 3 times with EA (30 ml). The organic phases were combined, washed with saturated brine, dried over anhydrous sodium sulfate, filtered, concentrated to dryness, and the residue was purified by preparative plate with unfolding agent ratio of DCM/MeOH=10/1 to afford the compound TPD5457-3 (100 mg, yellow solid, purity 98.017%), yield: 27.11%. LCMS (ESI) m/z calcd. for C₃₇H₄₀FN₅O₆ [M+H]⁺ 670.3; found 670.1; ¹H NMR (400 MHz, DMSO_d₆): δ=10.19 (s, 1H), 10.06 (s, 1H), 8.44 (d, J=5.2 Hz, 1H), 7.76 (d, J=8.9 Hz, 2H), 7.69-7.58 (m, 2H). 7.48 (s, 1H), 7.31 (s, 1H), 7.24-7.09 (m, 4H), 6.40 (d, J=5.2 Hz, 1H), 4.07 (q, J=7.1 Hz, 2H), 3.95 (s, 3H), 3.14 (br. s., 4H), 2.56 (br. s., 4H), 2.38-2.32 (m 4H), 1.82-1.65 (m, 2H), 1.48 (s, 4H), 1.19 (t, J=7.1 Hz, 3H). The compound structure is confirmed.

To a 25 ml single-necked vial was sequentially added compound TPD5457-3 (100 mg, 0.1493 mmol), EtOH (0.5 ml), H₂O (0.5 ml), and lithium hydroxide monohydrate (9.4 mg, 0.2239 mmol). the reaction was carried out at 25° C. for 2 h. The reaction solution was concentrated to dryness to give crude TPD5457-4 (100 mg, white solid). The reaction solution was concentrated to dryness to give the crude compound TPD5457-4 (100 mg, white solid, purity 91.427%), yield: 94.44%. LCMS (ESI) m/z calcd. for C₃₅H₃₆FN₅O₆ [M+H]⁺ 642.3; found 642.1. The compound structure is confirmed.

Compound TPD5457-4 (110 mg, 0.1714 mmol), DMF (6 ml), TEA (34.69 mg, 0.3428 mmol), HATU (130.34 mg, 0.3428 mmol) and (2S,4R)-1-((S)-2-(12-azacyclonyl)-3,3-dimethylbutanoyl)-4-hydroxy-N-(4-(4-methylthiazol-5-yl)benzyl)pyrrolidine-2-carboxamide hydrochloride (96.06 mg, 0.2056 mmol). The reaction was carried out at 25° C. for 16 hours. The reaction solution was poured into water (10 ml) and extracted three times with EA (5 ml). The organic phases were combined, washed with saturated brine, dried over anhydrous sodium sulfate, filtered, and concentrated to dryness, and the residue was purified by preparative plates with the unfolding agent ratio of DCM/MeOH=6/1 to give compound TPD005457 (30.1 mg, off-white solid, purity 96.484%), yield: 16.04%. LCMS (ESI) m/z calcd. for C₅₇H₆₄FN₉O₈S [M+H]⁺ 1054.5; found 1054.7; ¹H NMR (400 MHz, DMSO_d₆): δ=10.19 (s, 1H), 10.06 (s, 1H), 8.98 (s, 1H), 8.58 (t, J=5.9 Hz, 1H), 8.44 (d, J=5.2 Hz, 1H). 7.92 (br. s., 1H), 7.76 (d, J=8.9 Hz, 2H), 7.88-7.83 (m, 2H), 7.48 (s, 1H), 7.44-7.37 (q, J=8.3 Hz, 4H), 7.32 (s, 1H), 7.21 (d, J=8.9 Hz, 2H), 7.15 (t, J=8.9 Hz 2H), 6.40 (d, J=5.1 Hz, 1H), 5.15 (d, J=3.2 Hz, 1H), 4.57 (d, J=9.4 Hz, 1H), 4.47-4.12 (m, 2H), 4.36 (br. s., 1H), 4.25-4.19 (m, 1H), 3.98-3.92 (m, 3H), 3.71-3.61 (m, 2H), 3.15 (br. s., 4H), 2.56 (br. s., 2H), 2.44 (s, 4H), 2.39-2.27 (m, 3H), 2.22 (d, J=7.1 Hz, 1H), 2.07-1.99 (m, 1H), 1.94-1.87 (m, 1H), 1.72 (br. s. 2H), 1.47 (s, 4H), 1.23 (s, 1H), 0.98-0.93 (m, 9H). The compound structure is confirmed.

### Example 27: Synthesis of Compound TPD005488

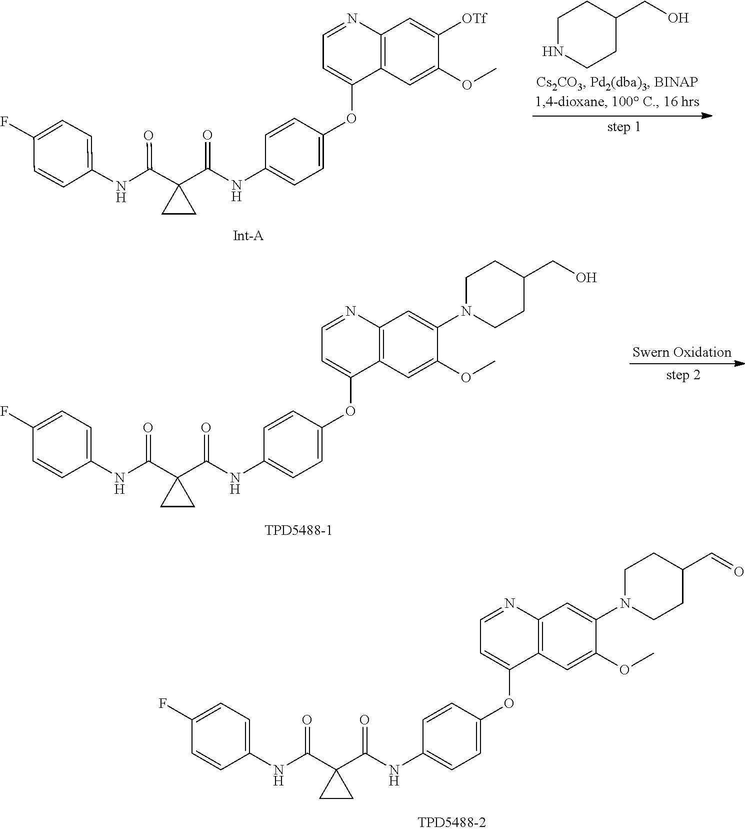 [SMILES: COc1cc2c(Oc3ccc(NC(=O)C4(C(=O)Nc5ccc(F)cc5)CC4)cc3)ccnc2cc1OS(=O)(=O)C(F)(F)F ; OCC1CCNCC1 ; COc1cc2c(Oc3ccc(NC(=O)C4(C(=O)Nc5ccc(F)cc5)CC4)cc3)ccnc2cc1N1CCC(CO)CC1 ; COc1cc2c(Oc3ccc(NC(=O)C4(C(=O)Nc5ccc(F)cc5)CC4)cc3)ccnc2cc1N1CCC(C=O)CC1]

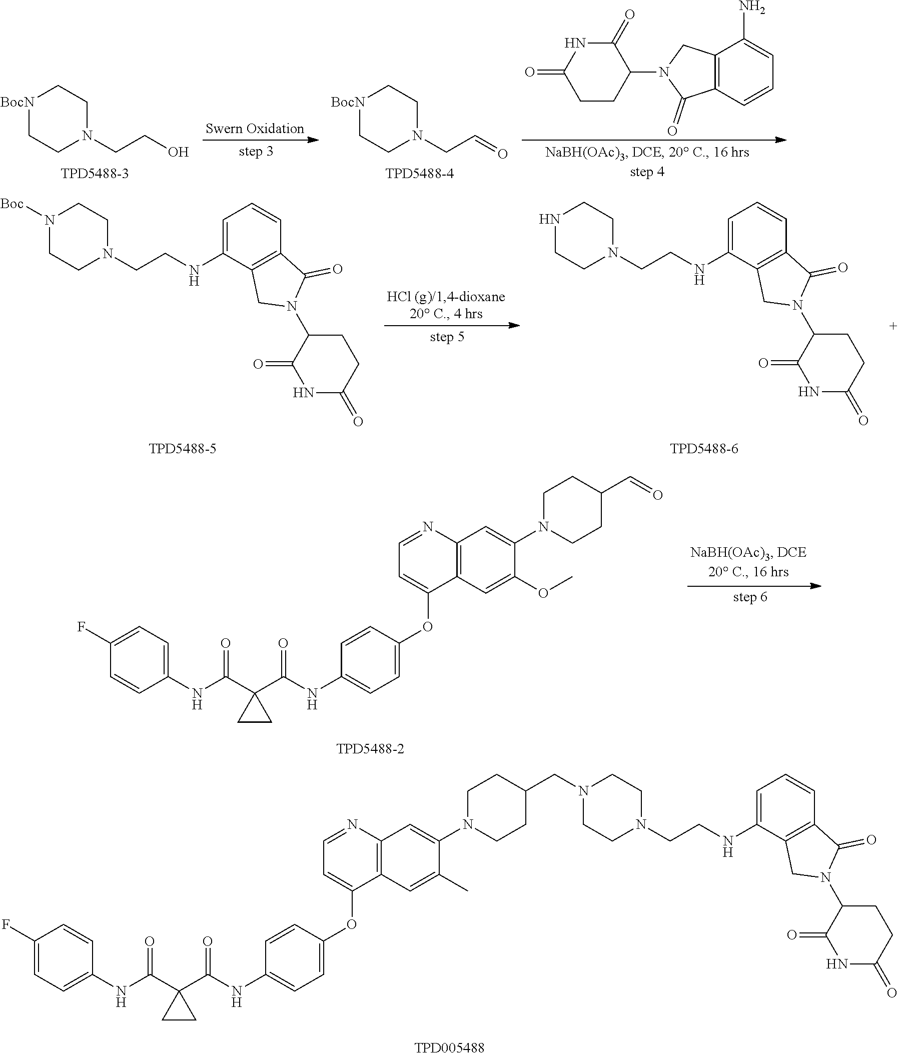

A 100 ml triple-necked vial was sequentially charged with compound int-A (600 mg, 0.9685 mmol), piperidine-4-methanol (245.4 mg, 2.1307 mmol), cesium carbonate (631.11 mg, 1.937 mmol), BINAP (241.22 mg, 0.3874 mmol), Pd₂ (dba)₃ (177.37 mg, 0.1937) and 1,4-dioxane (30 ml). The reaction was carried out at 100° C. for 16 h under nitrogen protection. After the reaction was completed, the reaction solution was reduced to room temperature and concentrated to dryness, and the residue was purified by silica gel column with eluent ratio of DCM/MeOH=50/1˜30/1 to afford the compound TPD5488-1 (360 mg, yellow solid, purity 63.777%), yield: 40.55%. LCMS (ESI) m/z calcd. for C₃₃H₃₃FN₄O₅ [M+H]⁺ 585.2; found 585.1; ¹H NMR (400 MHz, CDCl₃): δ=9.30 (s, 1H), 8.80 (s, 1H), 8.46 (d, J=5.2 Hz, 1H), 7.82 (d, J=8.8 Hz, 2H), 7.51-7.46 (m, 4H), 7.17 (d, J=8.8 Hz, 2H), 7.07-7.03 (m, 2H), 6.43 (d, J=5.2 Hz, 1H), 4.03 (s, 3H), 3.76-3.73 (m, 2H), 3.60 (d, J=6.4 Hz, 2H), 2.74-2.68 (m, 2H), 1.92-1.89 (m, 2H), 1.75-1.53 (m, 8H). It showed that the structure of the compound is correct.

A 50 ml triple-necked vial was charged with oxalyl chloride (86.85 mg, 0.6842 mmol) and DCM (5 ml). The reaction solution was cooled down to −78° C. under nitrogen protection and then DMSO (106.91 mg, 1.3684 mmol) was added dropwise. After half an hour of reaction at −78° C. a solution of DCM (5 ml) of compound TPD5488-2 (200 mg, 0.3421 mmol) was added dropwise. TEA (276.94 mg, 2.7368 mmol) was added dropwise after 1 h of reaction at −78° C. The reaction was carried out in a cold bath. After dropwise addition, the cold bath was withdrawn and the reaction was naturally warmed up for 1 hour. The reaction solution was poured into saturated aqueous sodium bicarbonate solution (20 ml) and extracted three times with DCM (20 ml). The organic phases were combined, washed with saturated brine, dried over anhydrous sodium sulfate, filtered, and concentrated to dryness to obtain the crude compound TPD5488-2 (80 mg, yellow solid, purity 53.406%), yield: 21.43%. LCMS (ESI) m/z calcd. for C₃₃H₃₁FN₄O₅ [M+H]⁺ 583.2; found 583.0. The compound structure is confirmed.

A 250 ml three-necked flask was charged with oxalyl chloride (5.51 g, 43.4 mmol) and DCM (80 ml). The reaction solution was cooled down to −78° C. under nitrogen protection and then DMSO (6.784 g, 86.8 mmol) was added dropwise. After half an hour of reaction at −78° C. a solution of DCM (20 ml) of compound tert-butyl 4-(2-hydroxyethyl)piperazine-1-carboxylate (5, 21.7 mmol) was added dropwise. The reaction was carried out at −78° C. for 1 hour followed by dropwise addition of TEA (17.47 g, 173.6 mmol). After dropwise addition, the cold bath was removed and the reaction was naturally warmed up for 1 hour. The reaction solution was poured into saturated aqueous sodium bicarbonate solution (100 ml), the organic phase was separated and the aqueous phase was extracted twice with DCM (80 ml). The organic phases were combined, washed with saturated brine, dried over anhydrous sodium sulfate, filtered, and concentrated to dryness to obtain the crude compound TPD5488-4 (4 g, light yellow oil, purity 90%), yield: 72.81%. LCMS (ESI) m/z calcd. for C₁₁H₂₀N₂O₃ [M+H]⁺ 229.2; found 229.2 and 247.2; ¹H NMR (400 MHz, DMSO_d₆): δ=9.58 (s, 1H), 3.28 (br. s., 1H), 3.21 (s, 3H), 2.41-2.39 (m, 6H), 1.40 (s, 9H). The compound structure is confirmed.

To a 250 ml triple-necked vial was sequentially added compound TPD5488-4 (1.76 g, 7.71 mmol), DCE (50 ml), 3-(4-amino-1-oxoisoindolin-2-yl)piperidine-2,6-dione (2 g, 7.71 mmol) and sodium triacetoxyborohydride (4.08 g, 19.2 mmol). The reaction was carried out under nitrogen protection at 20° C. for 16 hours. The reaction solution was decanted into saturated aqueous sodium bicarbonate solution (50 ml). The organic phase was separated and the aqueous phase was extracted twice with DCM (50 ml). The organic phases were combined, washed with saturated brine, dried over anhydrous sodium sulfate, filtered, and concentrated to dryness. The residue was purified by silica gel column with eluent ratio of DCM/MeOH=60/1˜40/1 to afford the compound TPD5488-5 (1.78 g, white solid, purity 92.874%), yield: 42.21%. LCMS (ESI) m/z calcd. for C₂₄H₃₃N₅O₅ [M+H]⁺ 472.3; found 472.1; ¹H NMR (400 MHz, CDCl₃): δ=8.20 (s, 1H), 7.39 (t, J=7.7 Hz, 1H), 7.30 (d, J=7.5 Hz, 1H), 6.80 (d, J=7.9 Hz, 1H), 5.26 (dd, J=13.3, 5.1 Hz, 1H), 4.33 (d, J=15.4 Hz. 1H), 4.25 (br. s., 1H), 4.16 (d, J=15.3 Hz, 1H), 3.46 (s, 4H), 3.29 (dd, J=10.4, 5.1 Hz, 2H), 2.96-2.85 (m, 2H), 2.71 (t, J=5.4 Hz, 2H), 2.48-2.32 (m, 5H), 2.27-2.17 (m, 1H), 1.48 (s, 9H). The compound structure is confirmed.

To a 50 ml single-necked vial was sequentially added compound TPD5488-5 (500 mg, 1.0581 mmol) and hydrogen chloride 1,4-dioxane solution (10 ml, 4 N). The reaction was carried out at 20° C. for 4 h. After completion of the reaction, it was concentrated to dryness to obtain the crude compound TPD5488-6 (480 mg, white solid), yield: 98.09%. LCMS (ESI) m/z calcd. for C₁₉H₂₅N₅O₃ [M+H]⁺ 372.2; found 372.0. The compound structure is confirmed.

To a 50 ml single-necked vial were sequentially added compound TPD5488-2 (80 mg, 0.1373 mmol), DCE (4 ml), compound TPD5488-6 (56.1 mg, 0.151 mmol) and sodium triacetoxyborohydride (87.3 mg, 0.4119 mmol). The reaction was carried out under nitrogen protection at 20° C. for 16 hours. After completion of the reaction, the reaction solution was concentrated to dryness. The residue was purified by preparative plate with the unfolding agent ratio of DCM/MeOH=10/1 to afford the compound TPD005488 (15.6 mg, light yellow solid, purity 99.304%), yield: 12.02%. LCMS (ESI) m/z calcd. for C₅₂H₅₆FN₉O₇ [M+H]⁺ 938.4; found 938.4; ¹H NMR (400 MHz, CDCl₃): δ=9.38 (s, 1H), 8.80 (s, 1H), 8.46 (d, J=5.3 Hz, 1H), 8.23 (br. s., 1H), 7.62 (d, J=8.9 Hz, 2H), 7.54-7.44 (m, 4H), 7.37 (t, J=7.7 Hz, 1H). 7.29-7.26 (m, 1H), 7.16 (d, J=8.9 Hz, 2H), 7.05 (t, J=8.6 Hz, 2H), 6.76 (d, J=7.9 Hz, 1H), 6.43 (d, J=5.3 Hz, 1H), 5.21 (dd, J=13.2, 5.0 Hz, 1H), 4.39-4.30 (m 2H), 4.02 (s, 3H), 3.71 (d, J=11.5 Hz, 2H), 3.35 (br. s., 2H), 2.96-2.57 (m, 12H), 2.45-2.18 (m, 5H), 1.90 (d, J=12.7 Hz, 3H), 1.80-1.61 (m, 6H), 1.59-1.44 (m 3H). The compound structure is confirmed.

### Example 28: Synthesis of Compound TPD005494

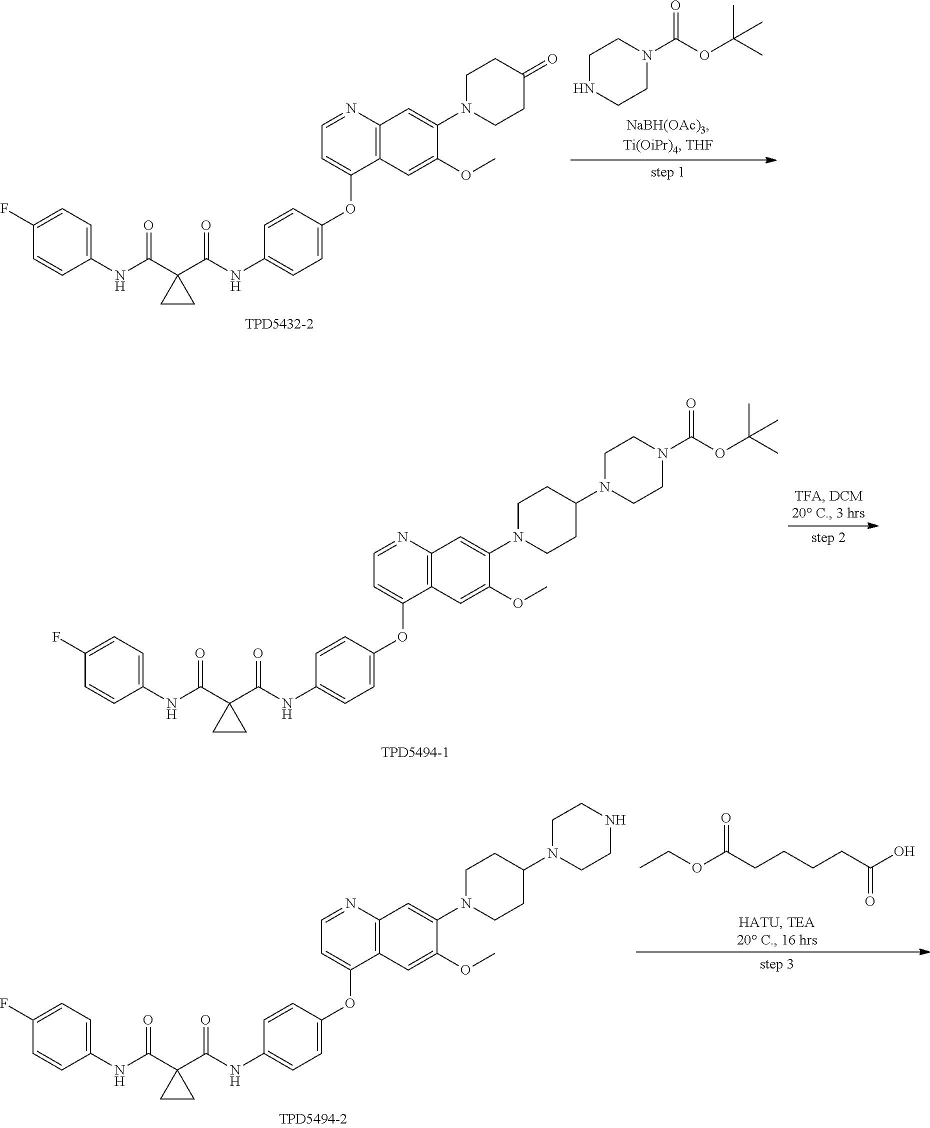

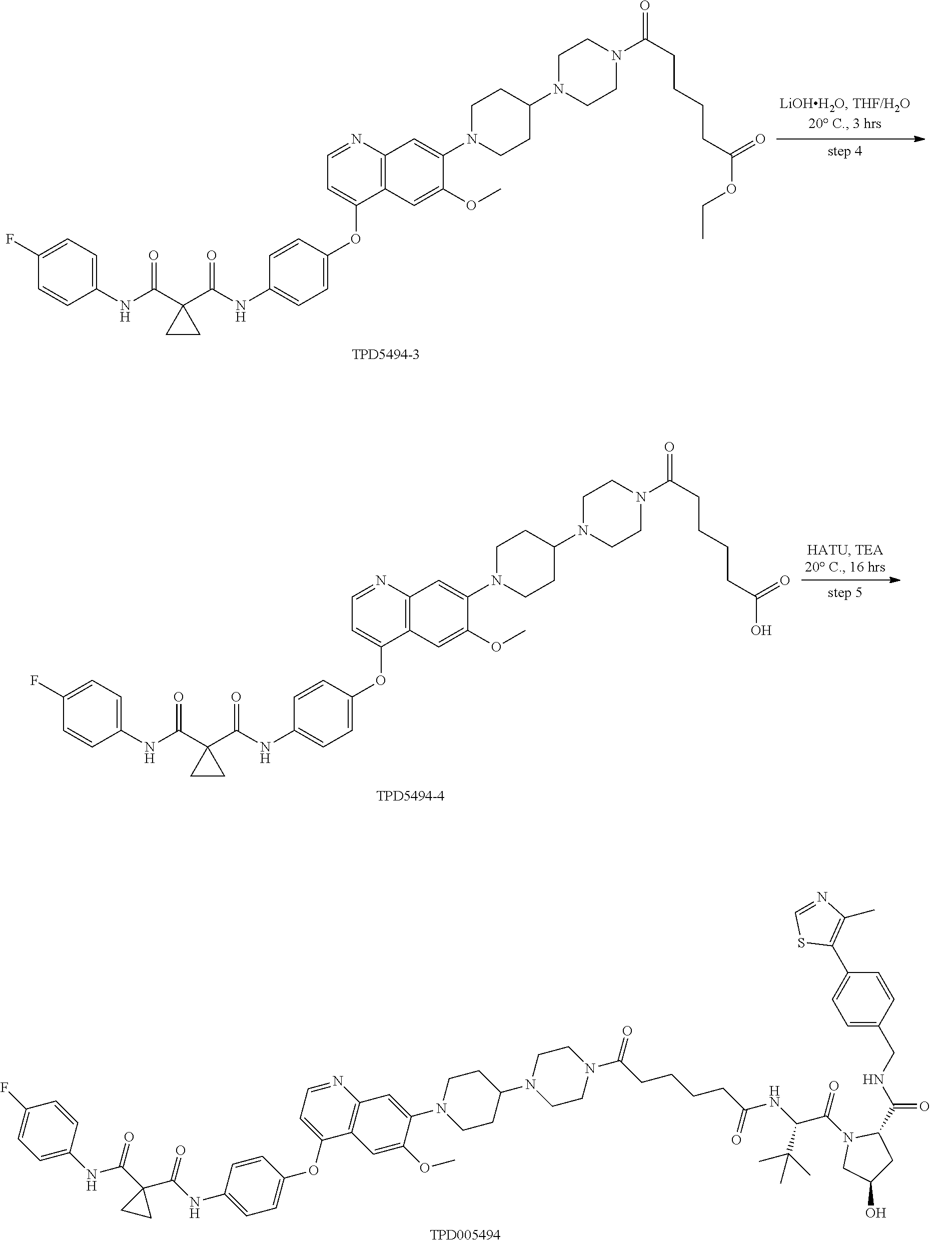 [SMILES: CCOC(=O)CCCCC(=O)N1CCN(C2CCN(c3cc4nccc(Oc5ccc(NC(=O)C6(C(=O)Nc7ccc(F)cc7)CC6)cc5)c4cc3OC)CC2)CC1 ; COc1cc2c(Oc3ccc(NC(=O)C4(C(=O)Nc5ccc(F)cc5)CC4)cc3)ccnc2cc1N1CCC(N2CCN(C(=O)CCCCC(=O)O)CC2)CC1 ; COc1cc2c(Oc3ccc(NC(=O)C4(C(=O)Nc5ccc(F)cc5)CC4)cc3)ccnc2cc1N1CCC(N2CCN(C(=O)CCCCC(=O)N[C@H](C(=O)N3C[C@H](O)C[C@H]3C(=O)NCc3ccc(-c4scnc4C)cc3)C(C)(C)C)CC2)CC1]

Compound TPD5432-2 (550 mg, 0.9673 mmol), 1-tert-butoxycarbonylpiperazine (900.8 mg, 4.8365 mmol), sodium triacetoxyborohydride (615.03 mg, 2.9019 mmol), tetraisopropyl titanate (1 ml), and DCE (10 ml) were added sequentially to a 50 ml triple-necked vial. The reaction mixture was stirred under nitrogen protection and at 40° C. for 16 hours. After the reaction was completed, the reaction solution was reduced to room temperature, water was added to the reaction solution and extracted three times with DCM (20 ml). The combined organic layers were washed with brine (50 ml), dried over anhydrous sodium sulfate, filtered and evaporated to give the residue. Purification of the residue by preparative TLC (DCM/MeOH=10/1) afforded the product compound TPD5494-1 (800 mg, yellow solid, purity 80.329%), yield: 89.92%. LCMS (ESI) m/z calcd. for C₄₁H₄₇FN₆O₆ [M+H]⁺ 739.4; found 739.5; ¹H NMR (400 MHz, CDCl₃): δ 9.21 (s, 1H), 8.68 (s, 1H), 8.40 (d, J=5.2 Hz, 1H), 7.55 (d, J=8.9 Hz, 2H), 7.49-7.33 (m, 4H), 7.10 (d, J=8.9 Hz, 2H), 7.04-6.90 (m, 2H), 6.36 (d, J=5.2 Hz, 1H), 3.95 (s, 3H), 3.73-3.65 (m, 3H), 3.39-3.38 (m, 5H), 2.74 (br. s., 8H), 2.51 (br. s., 4H), 1.61-1.60 (m, 6H), 1.40 (s, 9H). The compound structure is confirmed.

A 25 ml triple-necked vial was charged with compound TPD5494-1 (500 mg, 0.6767 mmol), DCM (2.5 ml), trifluoroacetic acid (2.5 ml). The obtained mixture reacted at 20° C. for 3 hours. After completion of the reaction, the yellow solid product compound TPD5494-2 (400 mg, yellow solid, purity 90.161%) was obtained by direct spin drying, yield: 83.43%. LCMS (ESI) m/z calcd. for C₃₆H₃₉FN₆O₄ [M+H]⁺ 639.3; found 639.3. The compound structure is confirmed.

Compound TPD54945-2 (400 mg, 0.6262 mmol), HATU (357.15 mg, 0.9393 mmol), monoethyl adipate (109.08 mg, 0.6262 mmol) and TEA (316.83 mg, 3.131 mmol) were added sequentially in a 25 ml single mouth vial. The reaction solution reacted for 16 hours at 20° C. under nitrogen protection. After the reaction was completed, water was added to the mixture and the mixture was extracted three times with DCM (5 ml). The combined organic layers were washed with brine (20 ml), dried over anhydrous sodium sulfate, filtered and evaporated to give the residue. Purification of the residue by preparative TLC (DCM/MeOH=10/1) afforded the yellow solid product compound TPD5494-3 (50 mg, yellow solid, purity 95.357%), yield: 9.58%. LCMS (ESI) m/z calcd. for C₄₄H₅₁FN₆O₇ [M+H]⁺ 795.4; found 795.5. The compound structure is confirmed.

To a 25 ml single-necked vial was added compound TPD5494-3 (50 mg, 0.0629 mmol), THF/MeOH/H₂O=1/1/1 (2 ml), LiOH·H₂O (5.28 mg, 0.1258 mmol). The resulting mixture was stirred at 20° C. for 3 hours. The reaction was completed, and the reaction solution was spun dry to give the crude product compound TPD5494-4 (48 mg, white solid, purity 96.029%), yield: 95.55%. LCMS (ESI) m/z calcd. for C₄₂H₄₇FN₆O₇ [M+H]⁺ 767.3; found 767.5. The compound structure is confirmed.

To a 50 ml single-necked vial were sequentially added DMF (5 ml), compound TPD5494-4 (48 mg, 0.0626 mmol), HATU (35.7 mg, 0.0939 mmol), (2S,4R)-1-((S)-2-amino-3,3-dimethylbutanoyl)-4-hydroxy-N-(4-(4-methylthiazol-5-yl) benzyl)pyrrolidine-2-carboxamide hydrochloride (35.84 mg, 0.0626 mmol) and TEA (31.67 mg, 0.313 mmol). The reaction was carried out under nitrogen protection at 20° C. for 16 hours. After completion of the reaction, the reaction solution was quenched with water. It was extracted three times with ethyl acetate (10 ml). The combined organic layers were washed with brine (20 ml), dried over anhydrous sodium sulfate, filtered and evaporated to give the residue. Purification of the residue by preparative TLC (DCM/MeOH=10/1) afforded the yellow solid product compound TPD005494 (13.5 mg, yellow solid, purity 95.005%), yield: 17.41%. LCMS (ESI) m/z calcd. for C₆₄H₇₅FN₁₀O₉S [M+H]⁺ 1079.5; found 1179.6; ¹H NMR (400 MHz, DMSO_d₆): δ 10.18 (s, 1H), 10.05 (s, 1H), 8.98 (s, 1H), 8.57 (t, J=6.1 Hz, 1H), 8.43 (d, J=5.2 Hz, 1H), 7.87 (d, J=9.3 Hz, 1H), 7.76 (d, J=8.8 Hz, 2H), 7.64 (dd, J=8.9, 5.0 Hz, 2H), 7.51-7.34 (m, 5H), 7.32 (s, 1H), 7.25-7.10 (m, 4H), 6.39 (d, J=5.2 Hz, 1H), 5.13 (d, J=3.4 Hz, 1H), 4.55 (d, J=9.3 Hz 1H), 4.44 (dd, J=15.5, 7.0 Hz, 2H), 4.35 (s, 1H), 4.21 (dd, J=15.9, 5.2 Hz, 1H), 3.94 (s, 3H), 3.66 (s, 4H), 3.44 (s, 4H), 2.64 (d, J=10.7 Hz, 2H), 2.45 (d, J=5.5 Hz, 5H), 2.29 (s, 3H), 2.19-1.97 (m, 3H), 1.94-1.81 (m, 3H), 1.63 (d, J=10.2 Hz, 2H), 1.47 (s, 9H), 1.23 (s, 1H), 0.94 (s, 9H). The compound structure is confirmed.

### Example 29: Synthesis of Compound TPD005495

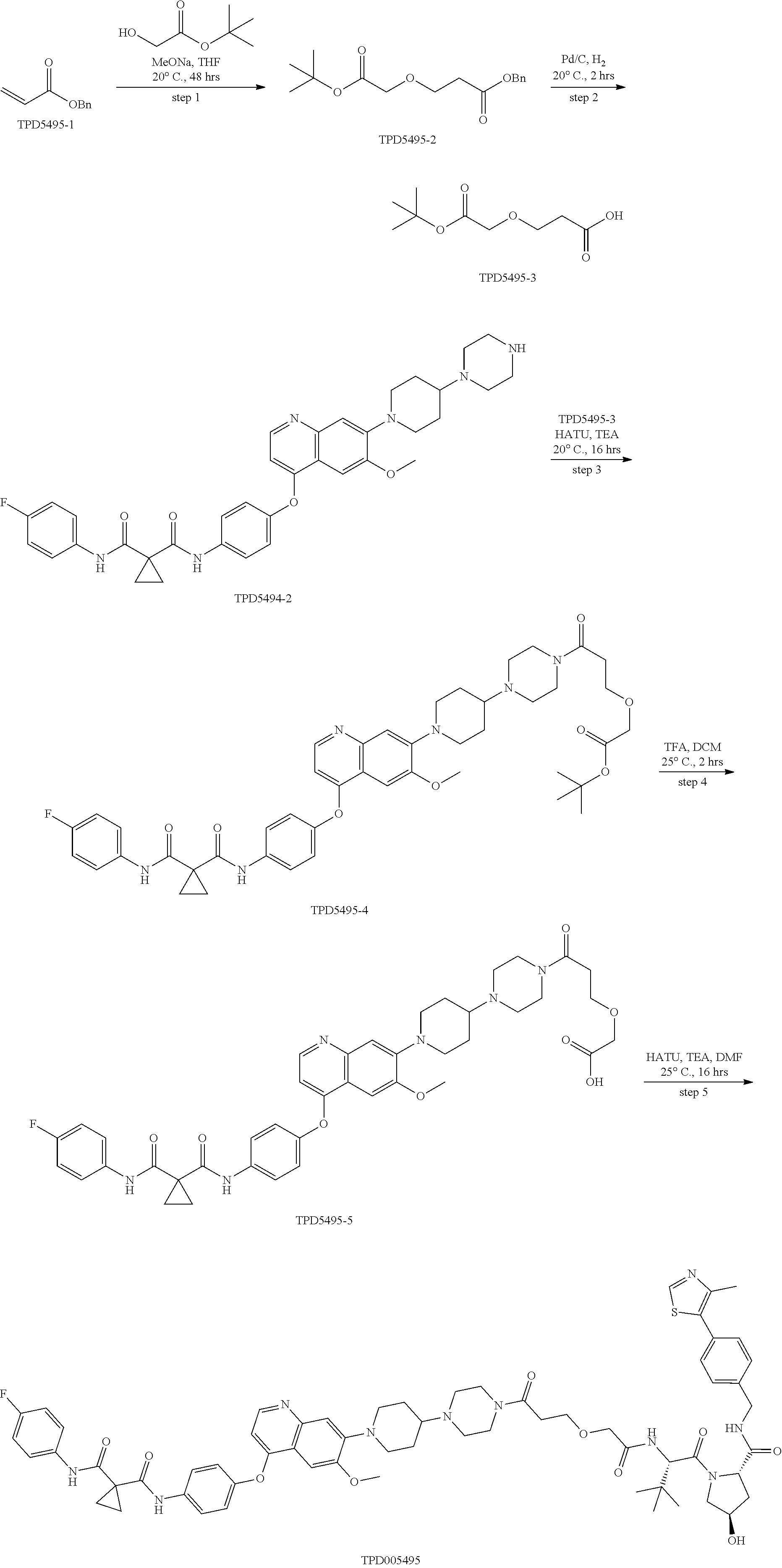

To a 100 ml triple-necked vial were sequentially added the compound TPD5495-1 (2 g, 12.33 mmol), tert-butyl 2-hydroxyacetate (1.63 g, 12.33 mmol), sodium methanol (1.33 g, 24.66 mmol) and tetrahydrofuran (50 ml). The reaction was carried out under nitrogen protection at 20° C. for 48 hours. After completion of the reaction, the reaction solution was quenched with saturated ammonium chloride (100 ml). The mixture was extracted three times with ethyl acetate (100 ml). The combined organic phases were washed with saturated saline (100 ml), dried over anhydrous sodium sulfate, filtered, and the filtrate was spun dry to give a residue. The residue was purified by silica gel column with eluent ratio of PE/EtOAc=50/1˜15/1 to afford the compound TPD5495-2 (800 mg, colorless oil, purity 88.706%), yield: 19.55%. LCMS (ESI) m/z calcd. for C₁₆H₂₂O₅ [M+H]⁺ 295.2; found 295.1 and 317.1 (plus Na); ¹H NMR (400 MHz, CDCl₃): δ=7.41-7.27 (m, 3H), 5.15 (s, 2H), 3.97 (s, 1H), 3.84 (t, J=6.4 Hz, 1H), 2.70 (t, J=6.4 Hz, 1H), 1.47 (s, 5H). The compound structure is confirmed.

To a 50 ml single-necked vial was added compound TPD5495-2 (200 mg, 0.68 mmol) and EA (2 ml). The addition of Pd/C (43 mg, 0.41 mmol) was continued and the reaction mixture was stirred at 20° C. for 2 h under H₂ environment. The mixture was filtered and concentrated to dryness to obtain the crude compound TPD5495-2 (100 mg, yellow solid, 90% purity), yield: 64.86%. LCMS (ESI) m/z calcd. for C₉H₁₆O₅ [M−H]⁻ 203.1; found 203.2. The compound structure is confirmed.

A 50 ml single-necked vial was sequentially charged with compound TPD5494-2 (172 mg, 0.27 mmol), compound TPD5494-2 (55 mg, 0.27 mmol), HATU (154 mg, 0.40 mmol), triethylamine (136 mg, 1.35 mmol) and DMF (5 ml). The reaction was carried out under nitrogen protection at 20° C. for 16 hours. After the reaction was completed, the reaction solution was poured into water (6 ml) and extracted three times with EA (6 ml). The organic phases were combined, washed with saturated brine, dried over anhydrous sodium sulfate, filtered, and concentrated to dryness. The residue was purified by preparative plate with unfolding agent ratio DCM/MeOH=5/1 to give compound TPD005495-4 (70 mg, light yellow solid, purity 90.63%), yield: 61.20%. LCMS (ESI) m/z calcd. for C₄₅H₅₃FN₆O₈ [M+H]⁺ 825.3; found 825.4 and 826.4; ¹H NMR (400 MHz, DMSO_d₆): δ=10.17 (s, 1H), 10.05 (s, 1H), 8.44 (d, J=5.2 Hz, 1H), 7.76 (d, J=8.9 Hz, 2H), 7.65-7.62 (m, 2H), 7.46 (s, 1H), 7.32 (s, 1H). 7.22-7.20 (m, 2H), 7.17-7.13 (m, 2H), 6.39 (d, J=5.2 Hz, 1H), 3.96 (s, 5H), 3.67-3.63 (d, J=11.3 Hz, 4H), 3.45 (m, 5H), 2.59-2.54 (t, J=11.2 Hz, 8H), 1.88 (m 2H), 1.64-1.62 (m, 2H), 1.47 (m, 4H), 1.47 (s, 4H), 1.42 (s, 9H). The compound structure is confirmed.

To a 50 ml single-necked vial were sequentially added compound TPD5495-4 (100 mg, 0.12 mmol), DCM (3 ml) and trifluoroacetic acid (1 ml). The reaction was carried out at 25° C. for 2 h. After completion of the reaction, it was concentrated to dryness to give the crude compound TPD5495-5 (90 mg, brown solid, purity 82.471%), yield: 79.62%. LCMS (ESI) m/z calcd. for C₄₁H₄₆FN₆O₈ [M+H]⁺ 769.3; found 385.2 and 769.3. The compound structure is confirmed.

A 25 ml single-necked vial was sequentially charged with compound TPD5495-5 (90 mg, 0.12 mmol), (2S,4R)-1-((S)-2-amino-3,3-dimethylbutanoyl)-4-hydroxy-N-(4-(4-methylthiazol-5-yl)benzyl)pyrrolidine-2-carboxamide hydrochloride (74 mg, 0.13 mmol), HATU (67 mg, 0.18 mmol), triethylamine (59 mg, 0.59 mmol) and DMF (5 ml). The reaction was carried out under nitrogen protection at 25° C. for 16 hours. After completion of the reaction, the reaction solution was poured into water (20 ml) and extracted three times with EA (20 ml). The organic phases were combined, washed with saturated brine, dried over anhydrous sodium sulfate, filtered, and concentrated to dryness. The residue was purified by preparative plate with unfolding agent ratio DCM/MeOH=8/1 to afford compound TPD005495 (15.5 mg, light yellow solid, purity 97.458%), yield: 10.93%. LCMS (ESI) m/z calcd. for C₆₃H₇₃FN₁₀O₁₀S [M+H]⁺ 1181.5; found 1181.3; ¹H NMR (400 MHz, DMSO_d₆): δ=10.16 (s, 1H), 10.05 (s, 1H), 8.99 (s, 1H), 8.59 (br. s., 1H), 8.44 (d, J=4.9 Hz, 1H), 7.74 (d, J=9.0 Hz, 2H), 7.46 (d, J=8.4 Hz, 3H). 7.40-7.38 (m, 5H), 7.22 (s, 1H), 7.17-7.13 (m, 4H), 6.40 (d, J=4.7 Hz, 1H), 5.14 (s, 1H), 4.58 (d, J=9.2 Hz, 1H), 4.39-4.35 (m, 3H), 4.27-4.25 (m, 2H), 3.94 (s, 5H), 3.73-3.64 (m, 6H), 3.46 (s, 4H), 2.66-2.63 (m, 4H), 2.49 (s, 3H), 2.07 (m, 2H), 2.02-1.82 (m, 4H), 1.59 (m, 3H), 1.47 (s, 4H), 1.23 (s, 1H), 0.93 (s, 9H). The compound structure is confirmed.

### Example 30: Synthesis of Compound TPD005496

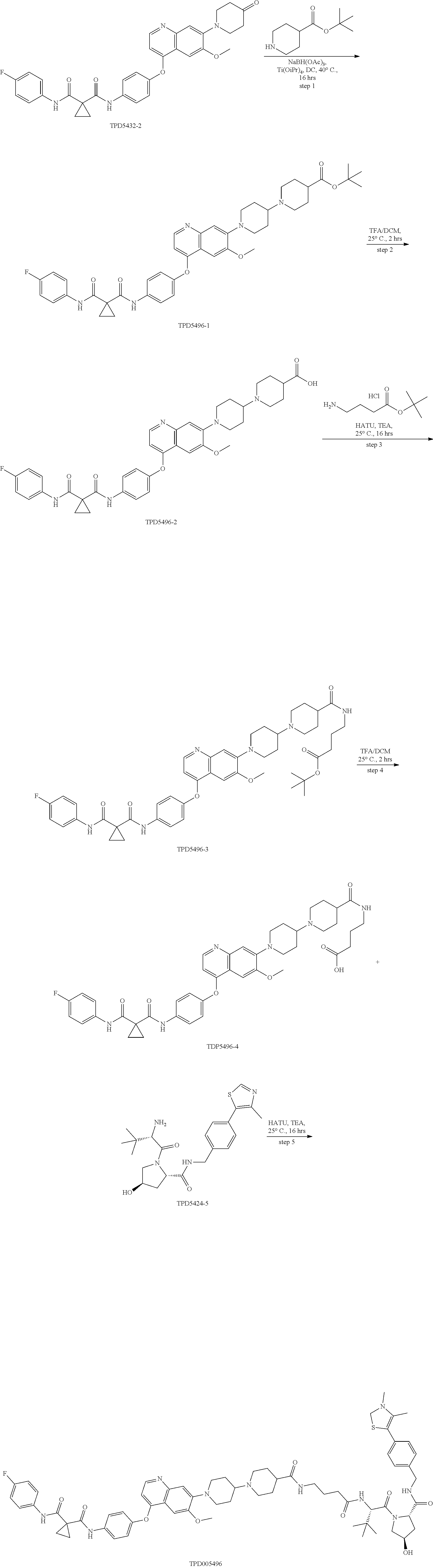

A 25 ml single-necked vial was charged sequentially with compound TPD5432-2 (350 mg, 0.6156 mmol), tert-butyl piperidine-4-carboxylate hydrochloride (651.67 mg, 3.078 mmol), sodium triacetoxyborohydride (391.41, 1.8468 mmol), tetraisopropyl titanate (1.5 ml) and 1,2-dichloroethane (5 ml). The reaction was carried out under nitrogen protection at 40° C. for 16 hours. After completion of the reaction, the reaction solution was brought to room temperature, decanted into water (10 ml) and extracted three times with DCM (5 ml). The organic phases were combined, washed with saturated brine, dried over anhydrous sodium sulfate, filtered, and concentrated to dryness. The residue was purified by silica gel column with eluent ratio of DCM/MeOH=100/1˜30/1 to afford the compound TPD5496-1 (500 mg, brown solid, purity 75.139%), yield: 82.72%. LCMS (ESI) m/z calcd. for C₄₂H₄₈FN₅O₆ [M+H]⁺ 738.4; found 738.4; ¹H NMR (400 MHz, DMSO_d₆): δ=10.17 (s, 1H), 10.06 (s, 1H), 8.43 (d, J=5.2 Hz, 1H), 7.75 (d, J=8.9 Hz, 2H), 7.66-7.61 (m, 2H), 7.46 (s, 1H), 7.31 (s, 1H). 7.23-7.19 (m, 2H), 7.18-7.12 (m, 2H), 6.39 (d, J=5.2 Hz, 1H), 3.95 (s, 3H), 3.65 (d, J=11.3 Hz, 2H), 2.92-2.85 (m, 4H), 2.64 (t, J=11.2 Hz, 2H), 2.46-2.43 (m 2H), 1.84-1.76 (m, 4H), 1.69-1.63 (m, 4H), 1.47 (s, 4H), 1.39 (s, 10H). The compound structure is confirmed.

To a 50 ml single-necked vial was sequentially added compound TPD5496-1 (450 mg, 0.6099 mmol), DCM (2 ml) and trifluoroacetic acid (2 ml). The reaction was carried out at 25° C. for 2 h. After completion of the reaction, it was concentrated to dryness to give the crude compound TPD5496-2 (450 mg, brown solid), yield: 81.41%. LCMS (ESI) m/z calcd. for C₃₈H₄₀FN₅O₆ [M+H]⁺682.3; found 682.2. The compound structure is confirmed.

To a 25 ml single-necked vial were sequentially added compounds TPD5496-2 (450 mg, 0.6601 mmol), H-Y-ABU-OTBU hydrochloride (155.01 mg, 0.7921 mmol), HATU (501.98 mg, 1.3202 mmol), triethylamine (133.59 mg, 1.3202 mmol) and DMF (5 ml). The reaction was carried out under nitrogen protection at 25° C. for 16 hours. After completion of the reaction, the reaction solution was poured into water (20 ml) and extracted three times with EA (10 ml). The organic phases were combined, washed with saturated brine, dried over anhydrous sodium sulfate, filtered, and concentrated to dryness. The residue was purified by silica gel column with eluent ratio of DCM/MeOH=50/1˜30/1 to afford the compound TPD5496-3 (250 mg, brown solid, purity 76.554%) yield: 35.24%. LCMS (ESI) m/z calcd. for C₄₆H₅₅FN₆O₇ [M+H]⁺ 823.4; found 823.4. The compound structure is confirmed.

To a 50 ml single-necked vial were sequentially added compound TPD5496-3 (250 mg, 0.3038 mmol), DCM (3 ml) and trifluoroacetic acid (2 ml). The reaction was carried out at 25° C. for 2 h. After completion of the reaction, it was concentrated to dryness to give the crude compound TPD5496-4 (200 mg, brown solid), yield: 71.49%. LCMS (ESI) m/z calcd. for C₄₂H₄₇FN₆O=[M+H]⁺ 767.4; found 767.3. The compound structure is confirmed.

A 25 ml single-necked vial was sequentially charged with compounds TPD5496-4 (200 mg, 0.2608 mmol), (2S,4R)-1-((S)-2-amino-3,3-dimethylbutanoyl)-4-hydroxy-N-(4-(4-methylthiazol-5-yl)benzyl)pyrrolidine-2-carboxamide hydrochloride (146.16 mg, 0.3129 mmol), HATU (198.33 mg, 0.5216 mmol), triethylamine (52.78 mg, 0.5216 mmol) and DMF (5 ml). The reaction was carried out under nitrogen protection at 25° C. for 16 hours. After completion of the reaction, the reaction solution was poured into water (20 ml) and extracted three times with EA (10 ml). The organic phases were combined, washed with saturated brine, dried over anhydrous sodium sulfate, filtered, and concentrated to dryness. The residue was purified by preparative plate with unfolding agent ratio DCM/MeOH=5/1 to give compound TPD005496 (10.4 mg, light yellow solid, purity 95.075%), yield: 3.22%. LCMS (ESI) m/z calcd. for C₆₄H₇₅FN₁₀O₉S [M+H]⁺ 1179.5; found 590.4 (half peak); ¹H NMR (400 MHz, DMSO_d₆): δ=10.20 (s, 1H), 10.05 (s, 1H), 8.99 (s, 1H), 8.57 (br. s., 1H), 8.45 (d, J=4.9 Hz, 1H), 7.92 (d, J=9.0 Hz, 2H), 7.76 (d, J=8.4 Hz, 2H). 7.66-7.63 (m, 2H), 7.50-7.36 (m, 6H), 7.22-7.13 (m, 4H), 6.41 (d, J=4.7 Hz, 1H), 5.14 (s, 1H), 4.55 (d, J=9.2 Hz, 1H), 4.45-4.36 (m, 3H), 4.25-4.19 (m, 1H). 3.96 (s, 3H), 3.73-3.57 (m, 5H), 3.05 (s, 3H), 2.72-2.67 (m, 2H), 2.45 (s, 3H), 2.33-2.16 (m, 4H), 2.07-1.91 (m, 9H), 1.62 (s, 2H), 1.48 (s, 4H), 1.23 (br. s. 3H), 0.95 (s, 9H). The compound structure is confirmed.

### Example 31: Synthesis of Compound TPD005505

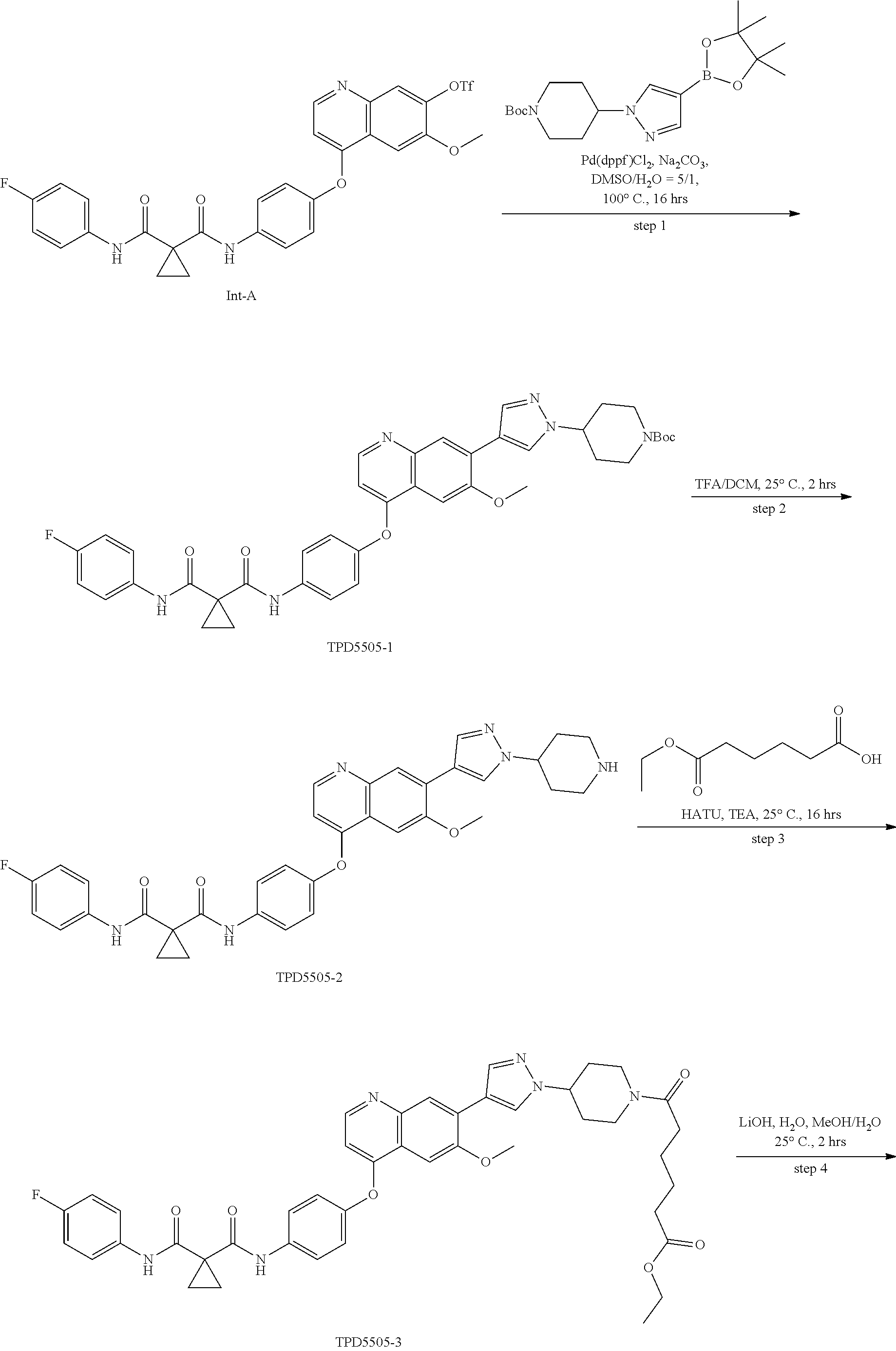

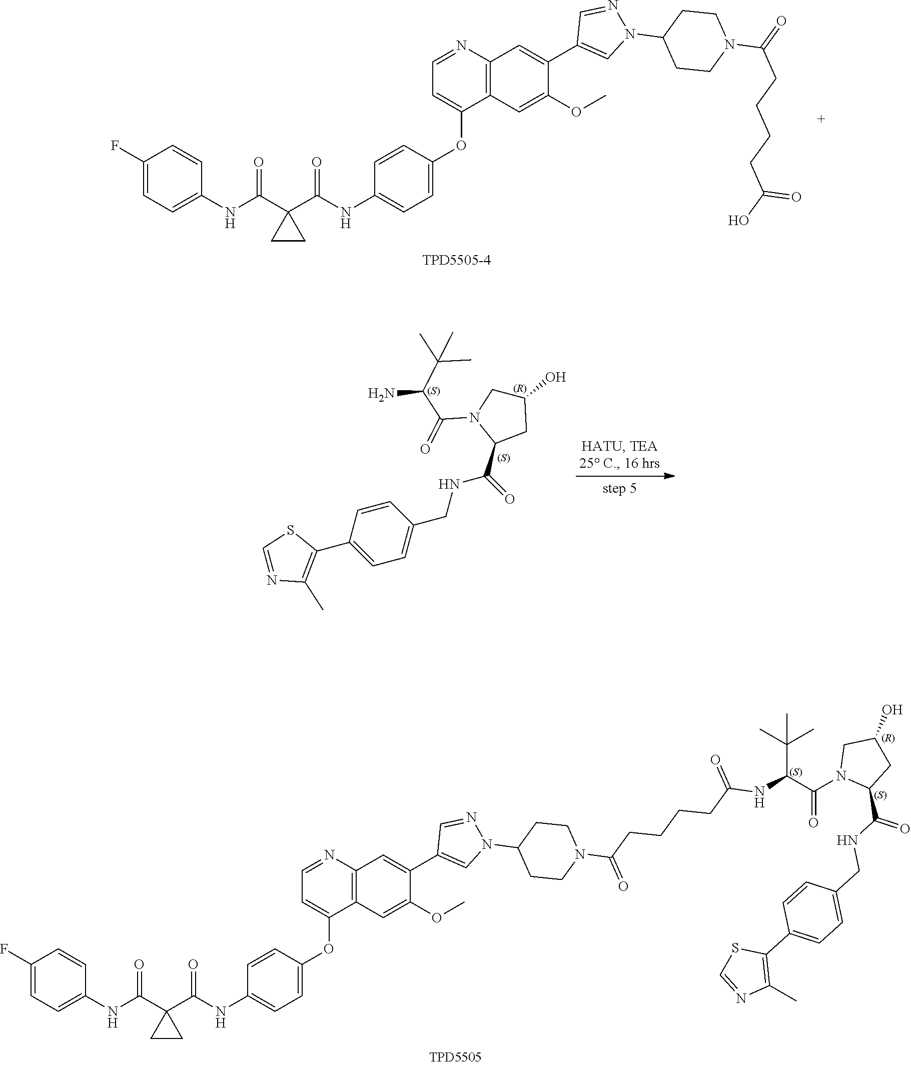

A 25 ml triple-necked vial was sequentially charged with compound int-A (300 mg, 0.4842 mmol), tert-butyl 4-[4-(4,4,5,5-tetramethyl-1,3,2-dioxaborolan-2-yl)-1H-pyrazol-1-yl]piperidine-1-carboxylate (274.03 mg, 0.7263 mmol), sodium carbonate (102.65 mg, 0.9684 mmol), Pd(dppf)Cl₂ (70.86 mg, 0.09684) and DMSO/H₂O=5/1 (6 ml). The reaction was carried out under nitrogen protection at 100° C. for 16 hours. After completion of the reaction, the reaction solution was brought to room temperature, decanted into water (20 ml) and extracted three times with EA (10 ml). The organic phases were combined, washed with saturated brine, dried over anhydrous sodium sulfate, filtered, and concentrated to dryness. The residue was purified by silica gel column with eluent ratio of DCM/MeOH=50/1˜30/1 to afford the compound TPD5505-1 (350 mg, brown solid, purity 87.238%), yield: 87.48%. LCMS (ESI) m/z calcd. for C₄₀H₄₁FN₆O₆ [M+H]⁺ 721.3; found 721.2; ¹H NMR (400 MHz, DMSO_d₆): δ=10.20 (s, 1H), 10.06 (s, 1H), 8.52 (d, J=5.1 Hz, 1H), 8.44 (s, 1H), 8.27 (s, 1H), 8.16 (s, 1H), 7.78 (d, J=8.9 Hz, 2H), 7.67-7.62 (i, 3H), 7.26 (d, J=9.0 Hz, 2H), 7.18-7.14 (m, 2H), 6.48 (d, J=5.1 Hz, 1H), 4.48-4.40 (m, 2H), 4.10-4.05 (m, 6H), 2.07-2.04 (m, 2H), 1.92-1.82 (in, 2H). 1.48 (s, 4H), 1.43 (s, 9H). The compound structure is confirmed.

To a 50 ml single-necked vial was sequentially added compound TPD5505-1 (300 mg, 0.4162 mmol), DCM (4 ml) and trifluoroacetic acid (2 ml). The reaction was carried out at 25° C. for 2 h. After completion of the reaction, it was concentrated to dryness to give the crude compound TPD5505-2 (320 mg, brown solid), yield: 77.73%. LCMS (ESI) m/z calcd. for C₃₈H₃₃FNO₄ [M+H]⁺ 621.3; found 621.4. The compound structure is confirmed.

A 25 ml single-necked vial was sequentially charged with compound TPD5505-2 (320 mg, 0.5156 mmol), monoethyl adipate (107.77 mg, 0.61872 mmol), HATU (392.09 mg, 1.0312 mmol), triethylamine (104.35 mg, 1.0312 mmol) and DMF (5 ml). The reaction was carried out under nitrogen protection at 25° C. for 16 hours. After completion of the reaction, the reaction solution was poured into water (20 ml) and extracted three times with EA (10 ml). The organic phases were combined, washed with saturated brine, dried over anhydrous sodium sulfate, filtered, and concentrated to dryness. The residue was purified by silica gel column with eluent ratio of DCM/MeOH=50/1˜30/1 to give compound TPD5505-3 (340 mg, brown solid, purity 94.211%), yield: 79.97%. LCMS (ESI) m/z calcd. for C₄₃H₄₅FN₆O₇ [M+H]⁺ 777.3; found 777.4; ¹H NMR (400 MHz, DMSO_d₆): δ=10.23 (s, 1H), 10.04 (s, 1H), 8.60 (d, J=5.5 Hz, 1H), 8.46 (s, 1H), 8.27 (s, 1H), 8.16 (s, 1H), 7.80 (d, J=9.0 Hz, 2H), 7.67-7.63 (m, 3H), 7.30-7.28 (m, 2H), 7.19-7.14 (m, 2H), 6.57 (d, J=5.5 Hz, 1H), 4.54-4.50 (m, 2H), 4.08-4.03 (m, 6H), 3.17 (s, 2H), 2.38 (t, J=6.9 Hz, 2H), 2.32 (t J=7.0 Hz, 2H), 2.12-2.06 (m, 2H), 1.99-1.93 (m, 1H), 1.86-1.80 (m, 1H), 1.58-1.54 (m, 4H), 1.48 (s, 4H), 1.18 (t, J=7.1 Hz, 3H). The compound structure is confirmed.

To a 50 ml single-necked vial was sequentially added compound TPD5505-3 (300 mg, 0.3862 mmol), lithium hydroxide monohydrate (32.41 mg, 0.7724 mmol) and methanol/water=2/1 (3 ml). The reaction was carried out at 25° C. for 2 h. After completion of the reaction, it was concentrated to dryness to give the crude compound TPD5505-4 (300 mg, brown solid), yield: 85.24%. LCMS (ESI) m/z calcd. for C₄₁H₄₁FN₆O₇ [M+H]⁺ 749.3; found 749.2. The compound structure is confirmed.

A 25 ml single-necked vial was sequentially charged with compound TPD5505-4 (300 mg, 0.4006 mmol), (2S,4R)-1-((S)-2-amino-3,3-dimethylbutanoyl)-4-hydroxy-N-(4-(4-methylthiazol-5-yl)benzyl)pyrrolidine-2-carboxamide hydrochloride (224.51 mg, 0.48072 mmol), HATU (304.64 mg, 0.8012 mmol), triethylamine (81.07 mg, 0.8012 mmol) and DMF (5 ml). The reaction was carried out under nitrogen protection at 25° C. for 16 hours. After completion of the reaction, the reaction solution was poured into water (20 ml) and extracted three times with EA (10 ml). The organic phases were combined, washed with saturated brine, dried over anhydrous sodium sulfate, filtered, and concentrated to dryness. The residue was purified by silica gel column with eluent ratio of DCM/MeOH=50/1˜30/1 to give compound TPD005505 (13.3 mg, white solid, purity 98.177%), yield: 2.80%. LCMS (ESI) m/z calcd. for C₆₃H₆₉FN₁₀O₉S [M+H]⁺ 1161.5; found 1161.6; ¹H NMR (400 MHz, DMSO_d₆): δ=10.20 (s, 1H), 10.05 (s, 1H), 8.98 (s, 1H), 8.59-8.52 (m, 2H), 8.43 (s, 1H), 8.26 (s, 1H), 8.15 (s, 1H), 7.88 (d, J=9.4 Hz, 1H), 7.78 (d, J=8.9 Hz, 2H), 7.67-7.60 (m, 3H), 7.43-7.37 (m, 5H), 7.26 (d, J=9.0 Hz, 2H), 7.18-7.13 (m, 2H), 6.49 (d, J=5.3 Hz, 1H), 5.13 (s, 1H), 4.57-4.51 (m 3H), 4.46-4.41 (m, 2H), 4.35 (s, 1H), 4.24-4.19 (m, 1H), 4.04 (s, 4H), 3.65 (br. s., 2H), 3.23-3.17 (m, 1H), 2.70 (d, J=25.1 Hz, 1H), 2.44 (s, 4H), 2.39-2.35 (m, 2H), 2.33-2.28 (m, 1H), 2.18-1.99 (m, 4H), 1.95-1.75 (m, 3H), 1.56-1.45 (m, 8H), 0.94 (s, 9H). The compound structure is confirmed.

### Example 32: Synthesis of compound TPD005706

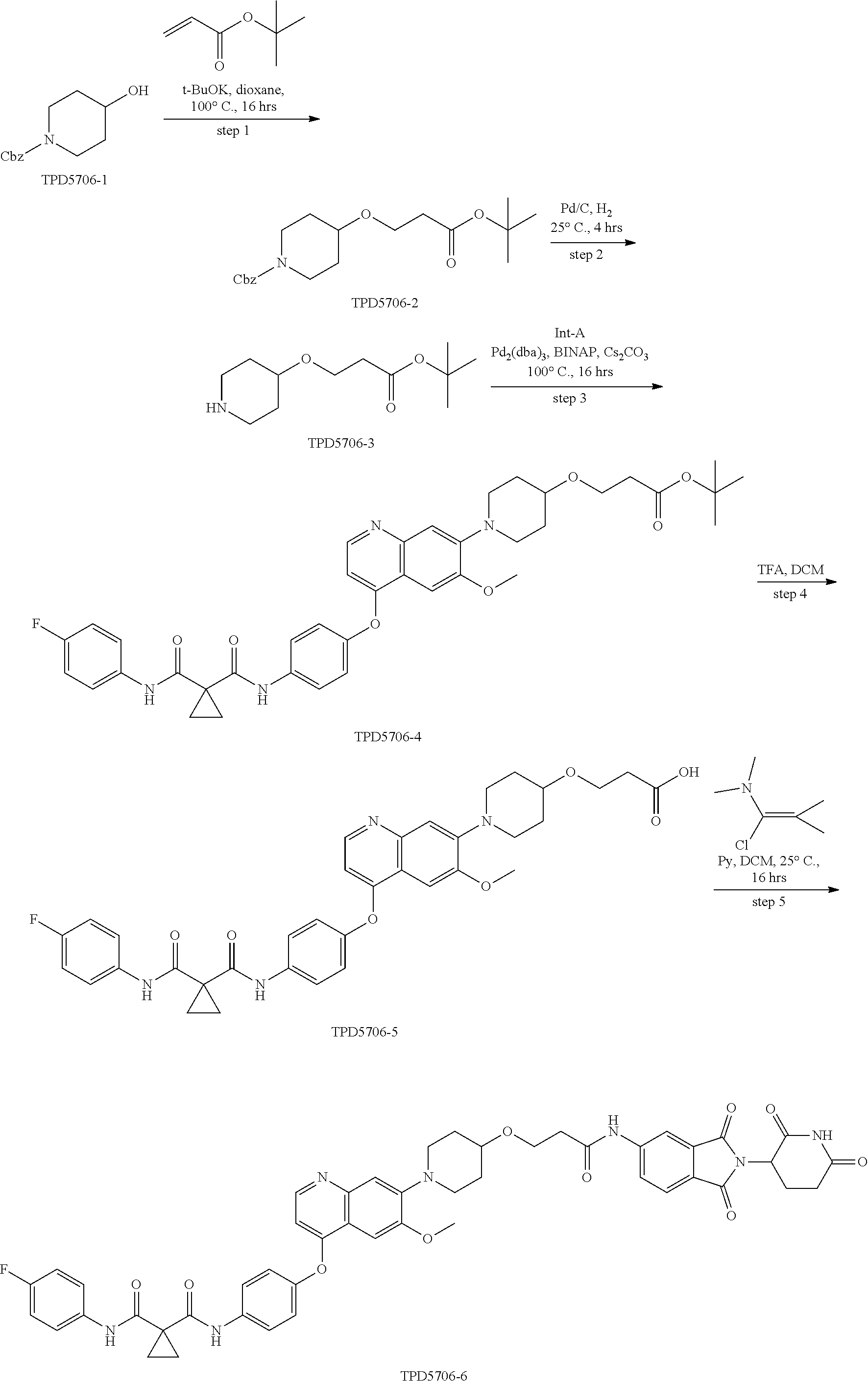

To a 100 ml triple-necked vial were sequentially added compound TPD5706-1 (5 g, 21.3 mmol), tert-butyl acrylate (4.10 g, 31.95 mmol), potassium tert-butoxide (0.48 g, 4.26 mmol) and 1,4-dioxane (50 ml). The reaction was carried out under nitrogen protection at 100° C. for 16 hours. After the reaction was completed, the reaction solution was reduced to room temperature and poured into saturated aqueous ammonium chloride solution (100 ml) and extracted three times with EA (50 ml). The organic phases were combined, washed with saturated brine, dried over anhydrous sodium sulfate, filtered, and concentrated to dryness to obtain the crude compound TPD5706-2 (1.4 g, yellow solid, purity 20.909%), yield: 3.76%. LCMS (ESI) m/z calcd. for C₂₀H₂₉NO₅ [M+H]⁺ 364.2; found 308.1. The compound structure is confirmed.

Compound TPD5706-2 (1.4 g, 3.9 mmol), THF (15 ml), and Pd/C (0.14 g, 10% Pd, 50% wet) were added sequentially in a 50 ml single-necked vial. The reaction was carried out for 4 h at 25° C. under hydrogen protection. After completion of the reaction, the reaction solution was filtered and concentrated to dryness to afford the crude compound TPD5706-3 (900 mg, brown oil, purity 37.009%), yield: 33.33%; LCMS (ESI) m/z calcd. for C₁₂H₂₃NO₃ [M+H]⁺ 230.2; found 230.1; ¹H NMR (400 MHz, DMSO_d₆): δ=3.58 (t, J=6.0 Hz, 2H), 3.29-3.25 (m, 1H), 3.13 (s, 2H), 2.87 (d, J=12.0 Hz, 2H), 2.44-2.41 (m, 1H), 2.38 (t, J=6.4 Hz, 2H), 1.78-1.74 (m, 2H), 1.40 (s, 9H), 1.26-1.17 (m, 2H). The compound structure is confirmed.

To a 100 ml triple-necked vial were sequentially added compounds TPD5706-3 (800 mg, 3.4886 mmol), int-A (2.161 g, 3.4886 mmol), cesium carbonate (2.27331 g, 6.9772 mmol), BINAP (868.9 mg, 1.3954 mmol), Pd₂ (dba)₃ (638.92 mg, 0.6977 mmol) and 1,4-dioxane (40 ml). The reaction was carried out under nitrogen protection at 100° C. for 16 hours. After the reaction was completed, the reaction solution was reduced to room temperature and concentrated to dryness, and the residue was purified by silica gel column with eluent ratio of DCM/MeOH=150/1˜30/1 to afford the compound TPD5706-4 (470 mg, brown solid, purity 72.189%), yield: 13.92%. LCMS (ESI) m/z calcd. for C₃₉H₄₃FN₄O₇ [M+H]⁺ 699.3; found 350.2 and 699.2; ¹H NMR (400 MHz, DMSO_d₆): δ 10.19 (s, 1H), 10.07 (s, 1H), 8.43 (d, J=5.2 Hz, 1H), 7.76 (d, J=8.8 Hz, 2H), 7.66-7.63 (m, 2H). 7.47 (s, 1H), 7.23-7.13 (m, 5H), 6.39 (d, J=5.2 Hz, 1H), 3.95 (s, 3H), 3.66 (t, J=6.0 Hz, 2H), 3.50 (s, 1H), 3.39 (s, 2H), 2.89 (t, J=9.2 Hz, 2H), 2.44 (t, J=6.0 Hz, 2H), 1.96 (s, 2H), 1.65-1.63 (m, 2H), 1.47 (s, 4H), 1.42 (s, 9H). The compound structure is confirmed.

To a 50 ml single-necked vial was sequentially added compound TPD5706-4 (400 mg, 0.5724 mmol), DCM (2 ml) and trifluoroacetic acid (2 ml). The reaction was carried out at 25° C. for 4 h. After completion of the reaction, it was concentrated to dryness to obtain the crude compound TPD5706-5 (380 mg, brown solid), yield: 57.95%. LCMS (ESI) m/z calcd. for C₃₅H₃₅FN₄O₇ [M+H]⁺ 643.3; found 643.4. The compound structure is confirmed.

To a 50 ml single-necked vial was sequentially added compound TPD5706-5 (100 mg, 0.1556 mmol), DCM (10 ml) and 1-chloro-N,N,2-trimethylpropenylamine (41.58 mg, 0.3112 mmol), and the mixture was stirred for 0.5 hr at 25° C. The mixture was then mixed with pyridine (36.92 mg, 0.4668 mmol) and 5-amino-2-(2,6-dioxopiperidin-3-yl). Then a DCM mixture of pyridine (36.92 mg, 0.4668 mmol) and 5-amino-2-(2,6-dioxopiperidin-3-yl)isodihydroindole-1,3-dione (85.04 mg, 0.3112 mmol) was added. The reaction was carried out under nitrogen protection at 25° C. for 16 hours. The reaction solution was decanted into water (10 ml). The organic phase was separated and the aqueous phase was extracted twice with DCM (5 ml). The organic phases were combined, washed with saturated brine, dried over anhydrous sodium sulfate, filtered, and concentrated to dryness. The residue was prepared by high performance liquid chromatography (HPLC) with the following parameters: column: sunfire 5 μm 19-150 mm; mobile phase: acetonitrile-water (0.1% FA); gradient: 20-55/8 min to afford the compound TPD005706 (11.6 mg, yellow solid, purity 98.769%) in the following yield: 7.97%. LCMS (ESI) m/z calcd. for C₄₈H₄₄FN₇O₁₀ [M+H]⁺ 898.3; found 449.6 and 898.3; ¹H NMR (400 MHz, DMSO_d₆): δ=11.13 (s, 1H), 10.67 (s, 1H), 10.19 (s, 1H), 10.06 (s, 1H), 8.42 (d, J=4.9 Hz. 1H), 8.28 (s, 1H), 8.24 (s, 0.5H), 7.93 (d, J=7.6 Hz, 1H), 7.86 (d, J=8.0 Hz, 1H), 7.76 (d, J=8.8 Hz, 2H), 7.66-7.63 (m, 2H), 7.45 (s, 1H), 7.25-7.13 (m, 5H). 6.38 (d, J=4.8 Hz, 1H), 5.15-5.10 (m, 1H), 3.93 (s, 3H), 3.82-3.80 (m, 2H), 3.55 (br. s., 2H), 2.89-2.84 (m, 3H), 2.69-2.66 (m, 2H), 2.62-2.57 (m, 2H), 2.01-1.98 (m, 3H), 1.65-1.63 (m, 2H), 1.47 (s, 3H), 1.23 (s, 2H). The compound structure is confirmed.

### Example 33: Synthesis of compound TPD005714

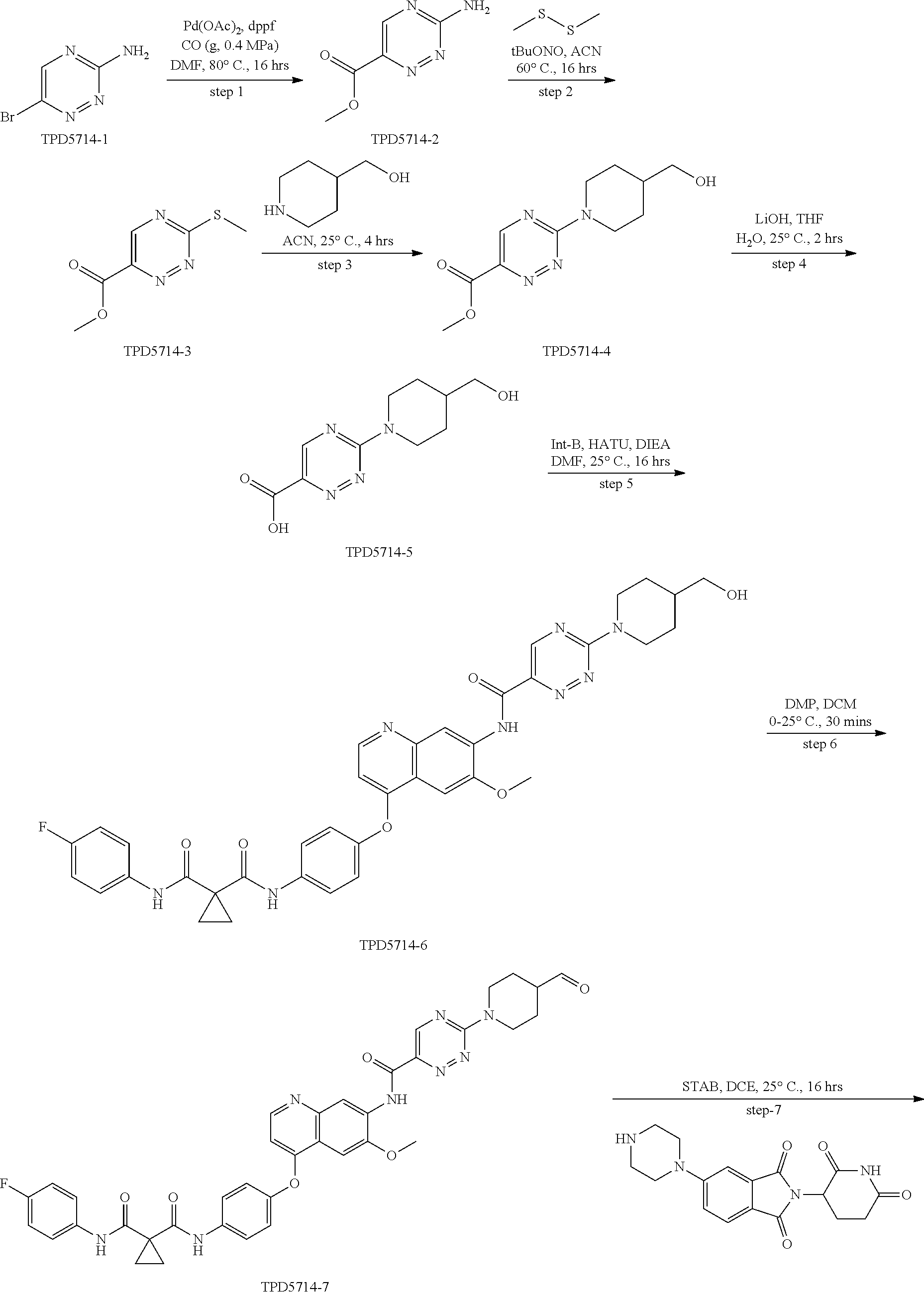

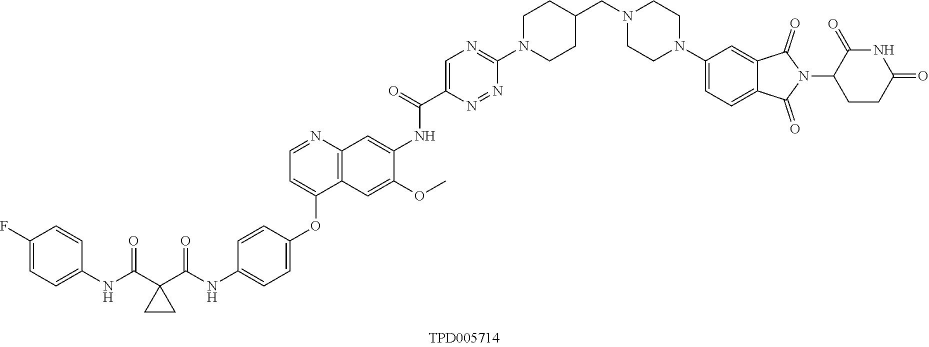 [SMILES: COc1cc2c(Oc3ccc(NC(=O)C4(C(=O)Nc5ccc(F)cc5)CC4)cc3)ccnc2cc1NC(=O)c1cnc(N2CCC(CN3CCN(c4ccc5c(c4)C(=O)N(C4CCC(=O)NC4=O)C5=O)CC3)CC2)nn1]

To a 500 ml autoclave was sequentially added compound TPD5714-1 (5 g, 28.6 mmol), palladium acetate (1.28 g, 5.72 mmol), dppf (3.17 g, 5.72 mmol)methanol (50 ml), triethylamine (10 ml) and DMF (150 ml). Carbonmonoxidegas at 0.4 MPa was flushed in and the reaction was carried out at 80° C. for 16 hours. After the reaction was completed, the reaction solution was filtered and the filtrate was spin-dried to obtain the residue. The residue was purified by silica gel column with the eluent ratio of DCM/MeOH=80/1˜60/1 to obtain the compound TPD5714-2 (2.4 g, brown solid, purity 70%), yield: 38.11%. LCMS (ESI) m/z calcd. for C₅H₆N₄O₂ [M+H]⁺ 155.1; found 155.1; ¹H NMR (400 MHz, CDCl₃): δ=8.83 (s, 1H), 6.02 (br. s, 2H), 4.06 (s, 3H). The compound structure is confirmed.

To a 100 ml single-necked vial were sequentially added the compounds TPD5714-2 (1.8 g, 7.82 mmol), ACN (30 ml), tert-butyl nitrite (4.84 g, 46.91 mmol) and dimethyl disulfide (7.36 g, 78.19 mmol). The reaction was carried out under nitrogen protection at 60° C. for 16 hours. After the reaction was completed, the reaction solution was concentrated to dryness. The residue was purified by silica gel column with eluent ratio of PE/EA=10/1˜5/1 to afford the compound TPD5714-2 (800 mg, light yellow oil, purity 83.445%), yield: 32.68%. LCMS (ESI) m/z calcd. for C₉H₁₆O₅[M−H]⁺186.0; found 186.0; ¹H NMR (400 MHz, CDCl₃): δ=8.95 (s, 1H), 4.08 (s, 3H), 2.83 (s, 3H). The compound structure is confirmed.

Compound TPD5494-3 (2 g, 10.80 mmol), piperidinemethanol (1.49 g, 12.94 mmol) and ACN (40 ml) were added sequentially in a 50 ml single-necked vial. The reaction was carried out under nitrogen protection at 25° C. for 5 hours. After the reaction was completed, the reaction solution was poured into water (150 ml) and extracted three times with EA (80 ml). The organic phases were combined, washed with saturated brine, dried over anhydrous sodium sulfate, filtered, and concentrated to dryness. The residue was purified by silica gel column with eluent ratio of PE/EA=5/1˜1/1 to give compound TPD005714-4 (310 mg, light yellow solid, purity 79.48%), yield: 9.26%. LCMS (ESI) m/z calcd. for C₁₁H₁₆N₄O₃ [M+H]⁺ 253.1; found 253.2; ¹H NMR (400 MHz, CDCl₃): δ=8.69 (s, 1H), 4.00 (s, 3H), 3.56 (d, J=5.9 Hz, 2H), 3.06 (br. s, 2H), 1.91-1.85 (m, 3H), 1.49-1.41 (m, 1H), 1.35-1.23 (m, 3H). The compound structure is confirmed.

To a 50 ml single-necked vial was sequentially added compound TPD5714-4 (140 mg, 0.56 mmol), LiOH (47 mg, 1.11 mmol) and THF (3 ml), MeOH (3 ml), H₂O (3 ml). The reaction was carried out at 25° C. for 2 h. After completion of the reaction, it was concentrated to dryness to give the crude compound TPD5714-5 (81 mg, yellow solid, purity 88.152%), yield: 54.00%. LCMS (ESI) m/z calcd. for C₁₀H₁₄N₄O₃ [M+H]⁺ 239.1; found 239.1. The compound structure is confirmed.

Compound TPD5714-5 (170 mg, 0.71 mmol), int-B (347 mg, 0.71 mmol), HATU (407 mg, 1.07 mmol), TEA (369 mg, 2.85 mmol), and DMF (3 mL) were added sequentially to a 50 ml single-necked vial. The reaction was carried out at 25° C. for 16 h. After completion of the reaction, the reaction solution was poured into water (10 ml) and extracted three times with EA (5 ml). The organic phases were combined, washed with saturated brine, dried over anhydrous sodium sulfate, filtered, and concentrated to dryness. High pressure preparation afforded the compound TPD5714-6 (85 mg, brown solid, purity 88.183%), yield: 14.87%. LCMS (ESI) m/z calcd. for C₃₇H₃₅FN₈O₆ [M+H]⁺ 707.3; found 707.4. The compound structure is confirmed.

To a 50 ml single-necked vial was sequentially added compound TPD5714-6 (70 mg, 0.10 mmol), Dess-Martin oxidizer (63 mg, 0.15 mmol) and DCM (3 ml). The reaction was carried out at 25° C. for 0.5 h. After completion of the reaction, the reaction solution was poured into saturated sodium bicarbonate solution (10 ml) and extracted three times with DCM (5 ml). The organic phases were combined, washed with saturated brine, dried over anhydrous sodium sulfate, filtered, and concentrated to dryness. The crude compound TPD5714-7 (35 mg, brown solid, purity 65.428%) was obtained, yield: 32.83%. LCMS (ESI) m/z calcd. for C₃₇H₃₄FN₈O₆ [M+H]⁺ 705.3; found 705.1. The compound structure is confirmed.

Compound TPD5714-7 (35 mg, 0.05 mmol), 2-(2,6-dioxopiperidin-3-yl)-5-(piperazin-1-yl)isoindoline-1,3-dione (18.8 mg, 0.05 mmol), sodium triacetoxyborohydride (21 mg, 0.09 mmol), and DCE (2 ml) were added sequentially to 25 ml of single-necked vials. The reaction was carried out under nitrogen protection at 25° C. for 16 hours. After completion of the reaction, the reaction solution was decanted into sodium bicarbonate (10 ml) and extracted three times with DCM (3 ml). The organic phases were combined, washed with saturated brine, dried over anhydrous sodium sulfate, filtered, and concentrated to dryness. The residue was purified by preparative plate with unfolding agent ratio DCM/MeOH=15/1 to give compound TPD005714 (3.0 mg, light yellow solid, purity 88.487%), yield: 5.23%. LCMS (ESI) m/z calcd. for Cs₄H₅₁FN₁₂O₉ [M+H]⁺ 1031.39; found 1031.60; ¹H NMR (400 MHz, CDCl₃:CD₃ OD=10:1): δ=9.39 (s, 1H), 8.97 (s, 1H), 8.70 (d, J=6.0 Hz, 1H), 7.76-7.73 (m, 4H), 7.56-7.49 (m, 3H), 7.23-7.19 (m, 2H), 7.15-7.11 (m 1H), 7.08-7.02 (m, 2H), 6.76-6.72 (m, 1H), 5.36-5.34 (m, 1H), 4.19 (s, 3H), 3.70-3.62 (m, 2H), 3.39-3.35 (m, 4H), 3.17-3.14 (m, 2H), 2.83-2.80 (m, 4H). 2.64-2.61 (m, 1H), 2.16-2.08 (m, 3H), 2.05-1.99 (m, 3H), 1.74-1.69 (m, 4H), 1.63-1.56 (m, 4H). The compound structure is confirmed.

### Example 34: Synthesis of Compound TPD005734

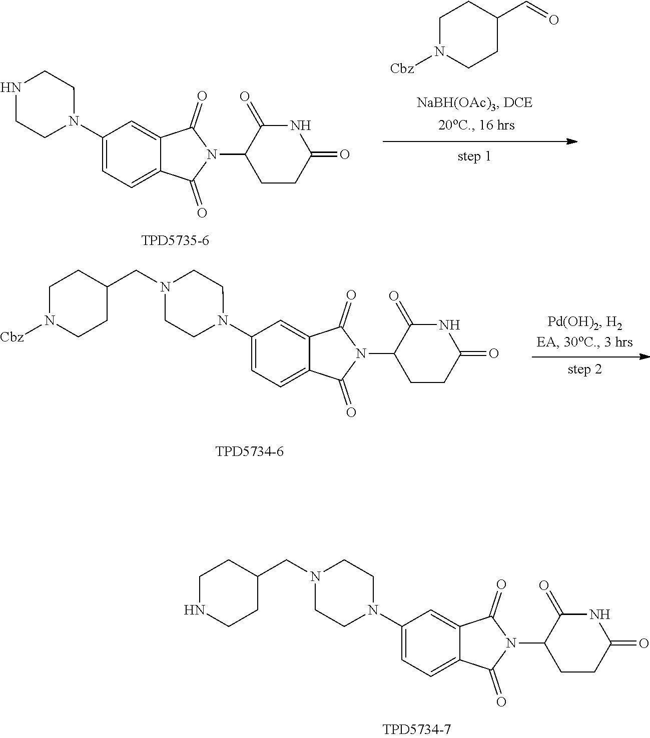 [SMILES: O=CC1CCN(C(=O)OCc2ccccc2)CC1 ; O=C1CCC(N2C(=O)c3ccc(N4CCNCC4)cc3C2=O)C(=O)N1 ; O=C1CCC(N2C(=O)c3ccc(N4CCN(CC5CCN(C(=O)OCc6ccccc6)CC5)CC4)cc3C2=O)C(=O)N1 ; O=C1CCC(N2C(=O)c3ccc(N4CCN(CC5CCNCC5)CC4)cc3C2=O)C(=O)N1]

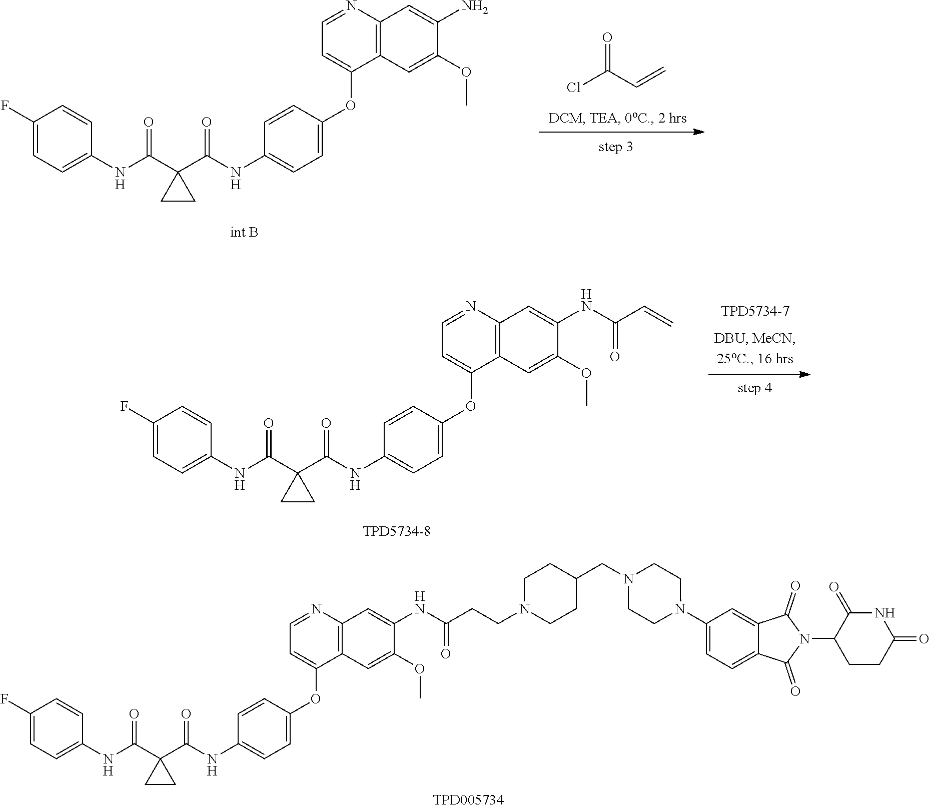

To a 50 ml triple-necked vial was sequentially added compound TPD5735-6 (500 mg, 1.3199 mmol), 4-formyl-N-CBZ piperidine (359.04 mg, 1.45189 mmol) and 1,2-dichloroethane (8 ml). Then sodium triacetoxyborohydride (839.22 mg, 3.9597 mmol) was added at room temperature. The reaction was then carried out at 25° C. for 16 hours. The reaction solution was poured into saturated aqueous sodium bicarbonate solution (20 ml) and extracted three times with DCM (5 ml). The organic phases were combined, washed with saturated brine, dried over anhydrous sodium sulfate, filtered, and concentrated to dryness. The residue was purified by preparative plate with unfolding agent ratio of DCM/MeOH=20/1 to give the crude compound TPD5734-6 (400 mg, yellow solid, 60% purity), yield: 31.7%. LCMS (ESI) m/z calcd. for C₃₁H₃₅N₅O₆ [M+H]⁺ 574.26; found 574.5. The compound structure is confirmed.

Compound TPD5734-6 (800 mg, 1.3946 mmol), palladium hydroxide (300 mg) and ethyl acetate (20 ml) were added sequentially to a 50 ml triple-necked vial. The reaction was then carried out at 30° C. under 20 psi hydrogen for 3 hours. The reaction solution was filtered, the filter cake was washed with ethyl acetate, and the filtrate was concentrated to dryness to obtain the crude compound TPD5734-7 (500 mg, yellow solid, purity 60%), yield: 48.95%. LCMS (ESI) m/z calcd. for C₂₃H₂₉N₅O₄ [M+H]⁺ 440.22; found 440.1. The compound structure is confirmed.

A 25 ml triple-necked vial was charged with int B (100 mg, 0.2056 mmol), triethylamine (83.22 mg, 0.8224 mmol) and dichloromethane (3 ml), tetrahydrofuran (3 ml). Then a solution of acryloyl chloride (37.22 mg, 0.4112 mmol) in dichloromethane (0.5 ml) was added dropwise at 0° C. The reaction was then carried out at 0° C. for 2 hours. The reaction solution was decanted into water (10 ml). It was extracted three times with DCM (3 ml). The organic phases were combined, washed with saturated brine, dried over anhydrous sodium sulfate, filtered, and concentrated to dryness. The residue was purified by preparative plate with unfolding agent ratio DCM/MeOH=15/1 to give compound TPD5734-8 (50 mg, yellow solid, purity 99.242%), yield: 44.65%. LCMS (ESI) m/z calcd. for C₃₀H₂₅FN₄O₅ [M+H]⁺ 541.18; found 542.2; ¹H NMR (400 MHz, DMSO_d₆): δ=10.20 (s, 1H), 10.06 (s, 1H), 9.72 (s, 1H), 8.88 (s, 1H), 8.52 (s, 1H), 7.78 (d, J=8.8 Hz, 2H), 7.66-7.63 (m, 2H), 7.61 (s, 1H), 7.25 (d, J=8.8 Hz, 2H), 7.16 (t, J=8.8 Hz, 2H), 6.90-6.83 (m, 1H), 6.47 (d, J=5.2 Hz, 1H), 6.36 (d, J=16.8 Hz, 1H), 5.81 (d, J=11.2 Hz, 1H), 4.05 (s, 3H), 1.48 (s, 4H). The compound structure is confirmed.

To a 25 ml triple-necked vial was sequentially added compound TPD5734-8 (45 mg, 0.0832 mmol), acetonitrile (3 ml), compound TPD5734-7 (36.57 mg, 0.0832 mmol) and DBU (38 mg, 0.2496 mmol). The reaction was carried out under nitrogen protection at 25° C. for 16 hours. The reaction solution was decanted into water (10 ml). It was extracted three times with DCM (3 ml). The organic phases were combined, washed with saturated brine, dried over anhydrous sodium sulfate, filtered, and concentrated to dryness. The residue was purified by preparative plate with unfolding agent ratio DCM/MeOH=10/1 to give compound TPD005734 (10.2 mg, yellow solid, purity 97.581%), yield: 12.2%. LCMS (ESI) m/z calcd. for C₅₃H₅₄FN₉O₉ [M+H]⁺ 980.40; found 980.3; ¹H NMR (400 MHz, DMSO_d₆): δ=11.09 (s, 1H), 10.82 (s, 1H), 10.20 (s, 1H), 10.06 (s, 1H), 8.92 (s, 1H), 8.49 (d, J=4.8 Hz, 1H), 7.77 (d, J=8.4 Hz, 2H), 7.69-7.63 (m, 3H)), 7.59 (s, 1H), 7.35 (s, 1H), 7.27-7.23 (m, 3H), 7.16 (t, J=8.8 Hz, 2H), 6.45 (d, J=4.8 Hz, 1H), 5.10-5.04 (m, 1H), 4.05 (s, 3H), 3.45 (s, 3H), 3.05 (br. s., 2H). 2.93-2.83 (m, 2H), 2.64 (br. s., 8H), 2.25 (br. s., 2H), 2.01 (br. s., 3H), 1.80 (d, J=10.4 Hz, 2H), 1.63 (br. s., 1H), 1.47 (s, 4H), 1.31-1.24 (m, 4H). The compound structure is confirmed.

### Example 35: Synthesis of Compound TPD005735

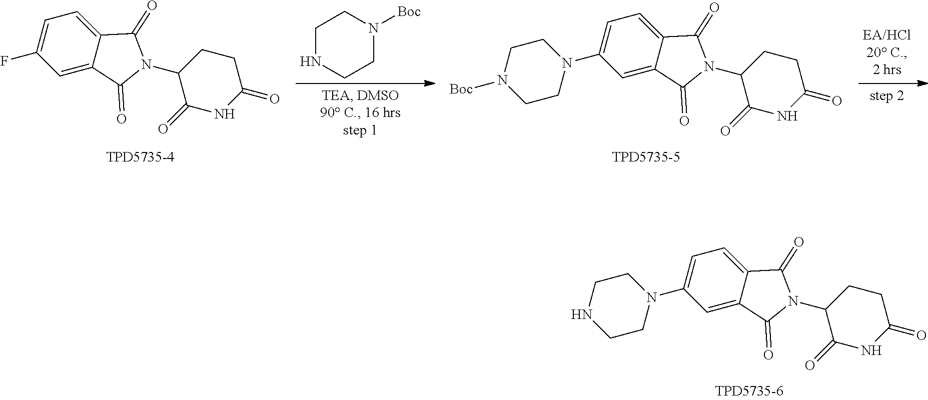 [SMILES: O=C1CCC(N2C(=O)c3ccc(F)cc3C2=O)C(=O)N1 ; CC(C)(C)OC(=O)N1CCNCC1 ; CC(C)(C)OC(=O)N1CCN(c2ccc3c(c2)C(=O)N(C2CCC(=O)NC2=O)C3=O)CC1 ; O=C1CCC(N2C(=O)c3ccc(N4CCNCC4)cc3C2=O)C(=O)N1]

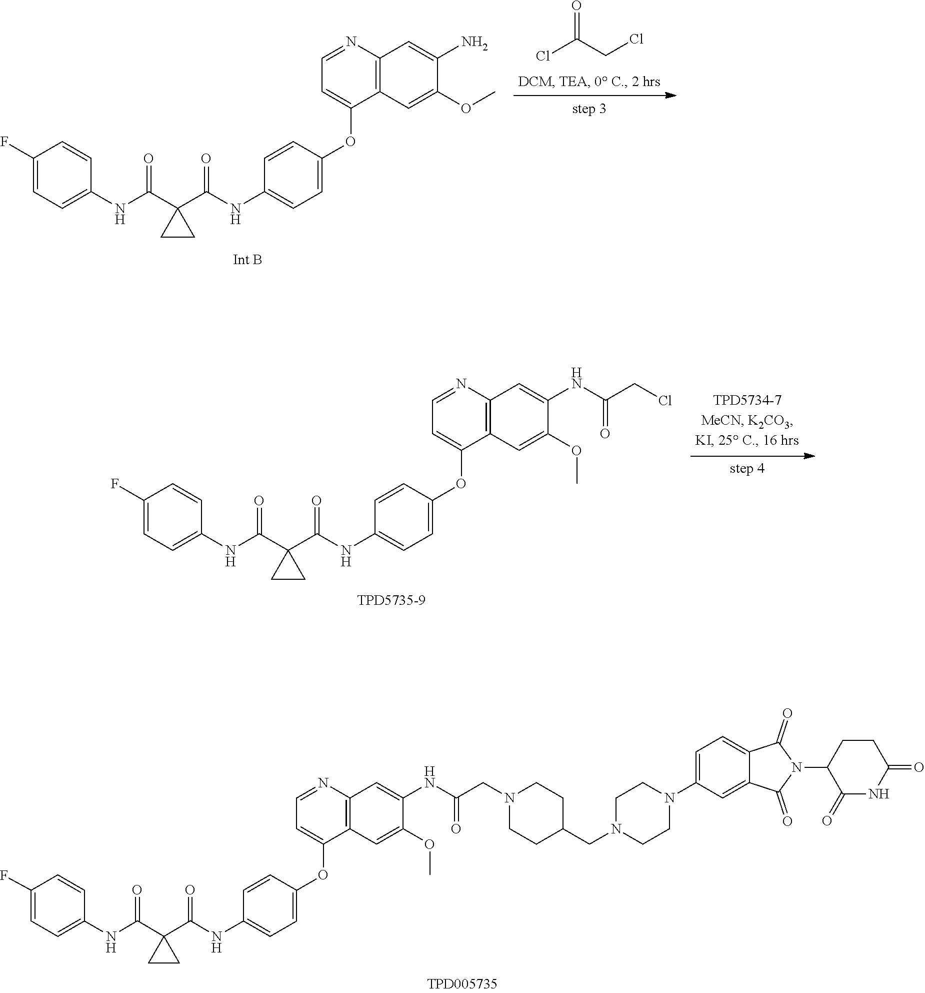 [SMILES: COc1cc2c(Oc3ccc(NC(=O)C4(C(=O)Nc5ccc(F)cc5)CC4)cc3)ccnc2cc1N ; O=C(Cl)CCl ; COc1cc2c(Oc3ccc(NC(=O)C4(C(=O)Nc5ccc(F)cc5)CC4)cc3)ccnc2cc1NC(=O)CCl ; COc1cc2c(Oc3ccc(NC(=O)C4(C(=O)Nc5ccc(F)cc5)CC4)cc3)ccnc2cc1NC(=O)CN1CCC(CN2CCN(c3ccc4c(c3)C(=O)N(C3CCC(=O)NC3=O)C4=O)CC2)CC1]

To a 100 ml triple-necked vial were sequentially added compound TPD5735-4 (4 g, 0.0145 mol), 1-tert-butoxycarbonylpiperazine (3.26 g, 0.0174 mol), triethylamine (2.93 g, 0.029 mol) and dimethylsulfoxide (50 ml). The reaction was then carried out at 90° C. for 16 hours. The reaction solution was poured into water (100 ml), filtered, the filter cake was washed with a small amount of water, and the filter cake was purified by silica gel chromatography column with eluent and ratio of PE/EtOAc=3/1˜1/1 to obtain the compound TPD5735-5 (2.5 g, yellow solid, purity 98.803%), yield: 38.62%. LCMS(ESI) m/z calcd. for C₂₂H₂₆N₄O₆ [M+H]⁺ 443.19; found 343.0, 387.0; ¹H NMR (400 MHz, DMSO_d₆): δ=11.09 (s, 1H), 7.70 (d, J=8.4 Hz, 1H), 7.35 (s, 1H), 7.25 (d, J=8.8 Hz, 1H), 5.10-5.06 (m, 1H), 3.47 (s, 8H), 2.93-2.84 (m, 1H), 2.61-2.50 (m, 2H), 2.03-1.99 (m, 1H), 1.43 (s, 9H). The compound structure is confirmed.

To a 100 ml triple-necked flask was sequentially added compound TPD5735-5 (2.5 g, 0.0056 mol), ethyl acetate (10 ml) and ethyl acetate hydrochloric acid gas (50 ml). The reaction was then carried out at 25° C. for 16 hours. The reaction solution was filtered and the filter cake was dried to obtain the crude compound TPD5735-6 (1.9 g, yellow solid, purity 75.193%), yield: 67.86%. LCMS (ESI) m/z calcd. for C₁₇H₁₈N₄O₄ [M+H]⁺ 343.13; found 343.0; ¹H NMR (400 MHz, DMSO_d₆): δ=11.10 (s, 1H), 9.28 (s, 2H), 7.75 (d, J=8.4 Hz, 1H), 7.45 (s, 1H), 7.33 (d, J=8.8 Hz, 1H), 5.12-5.07 (m, 1H), 3.57 (s, 4H), 3.21 (s, 4H), 2.93-2.84 (m, 1H), 2.67-2.53 (m, 2H), 2.04-1.99 (m, 1H). The compound structure is confirmed.

Compound int B (100 mg, 0.2056 mmol), triethylamine (83.22 mg, 0.8224 mmol), tetrahydrofuran (3 mL), and dichloromethane (3 mL) were added sequentially to a 25 mL triple-necked vial. A solution of chloroacetyl chloride (34.85 mg, 0.3084 mmol) in dichloromethane (0.5 mL) was then added dropwise at 0° C. The reaction was then carried out at 0° C. for 2 hours. The reaction solution was poured into water (10 ml) and extracted three times with DCM (3 ml). The organic phases were combined, washed with saturated brine, dried over anhydrous sodium sulfate, filtered, and concentrated to dryness. The residue was purified by preparative plate with the unfolding agent ratio of DCM/MeOH=15/1 to afford the compound TPD5735-9 (70 mg, yellow solid, purity 98.963%), yield: 59.85%. LCMS (ESI) m/z calcd. for C₂₉H₂₄ClFN₄O₅ [M+H]⁺ 563.14; found 563.0; ¹H NMR (400 MHz, DMSO_d₆): δ=10.19 (s, 1H), 10.05 (s, 1H), 9.84 (s, 1H), 8.79 (s, 1H), 8.52 (d, J=5.2 Hz, 1H), 7.77 (d, J=8.8 Hz, 2H), 7.66-7.63 (m, 3H), 7.25 (d, J=8.8 Hz, 2H), 7.15 (t, J=8.8 Hz, 2H), 6.48 (d, J=4.8 Hz, 1H), 4.53 (s, 2H), 4.06 (s, 3H), 1.48 (s, 4H). The compound structure is confirmed.

To a 25 ml triple-necked vial was sequentially added compound TPD5735-9 (65 mg, 0.1155 mmol), compound TPD5734-7 (50.76 mg, 0.1155 mmol), potassium carbonate (31.88 mg, 0.231 mmol), potassium iodide (1.92 mg, 0.01155 mmol) and acetonitrile (3 ml). The reaction was then carried out at 25° C. for 16 hours. The reaction solution was filtered and concentrated to dryness. The residue was purified by preparative plate with unfolding agent ratio DCM/MeOH=15/1 to give compound TPD005735 (8.85 mg, yellow solid, purity 95.813%), yield: 7.62%. LCMS (ESI) m/z calcd. for C₅₂H₅₂FN₉O₉ [M+H]⁺ 966.39, 967.39; found 966.3, 967.3; ¹H NMR (400 MHz, DMSO_d₆): δ 11.09 (s, 1H), 10.27 (s, 1H), 10.20 (s, 1H), 10.06 (s, 1H), 8.86 (s, 1H), 8.51 (d, J=5.2 Hz, 1H), 7.78 (d, J=8.8 Hz, 2H)), 7.69-7.63 (m, 4H), 7.35 (s, 1H), 7.28-7.24 (m, 3H), 7.16 (t, J=9.2 Hz, 2H), 6.47 (d, J=5.2 Hz, 1H), 5.10-5.05 (dd, J=5.2, 5.6 Hz, 1H), 4.09 (s, 3H), 3.45 (s 4H), 3.21 (s, 2H), 2.92-2.85 (m, 3H), 2.67-2.55 (m, 5H), 2.30-2.25 (m, 4H), 2.03-2.00 (m, 1H), 1.82 (d, J=10.4 Hz, 2H), 1.62 (br. s., 1H), 1.48 (s, 4H), 1.30-1.22 (m, 3H). The compound structure is confirmed.

### Example 36: Synthesis of Compound TPD005736

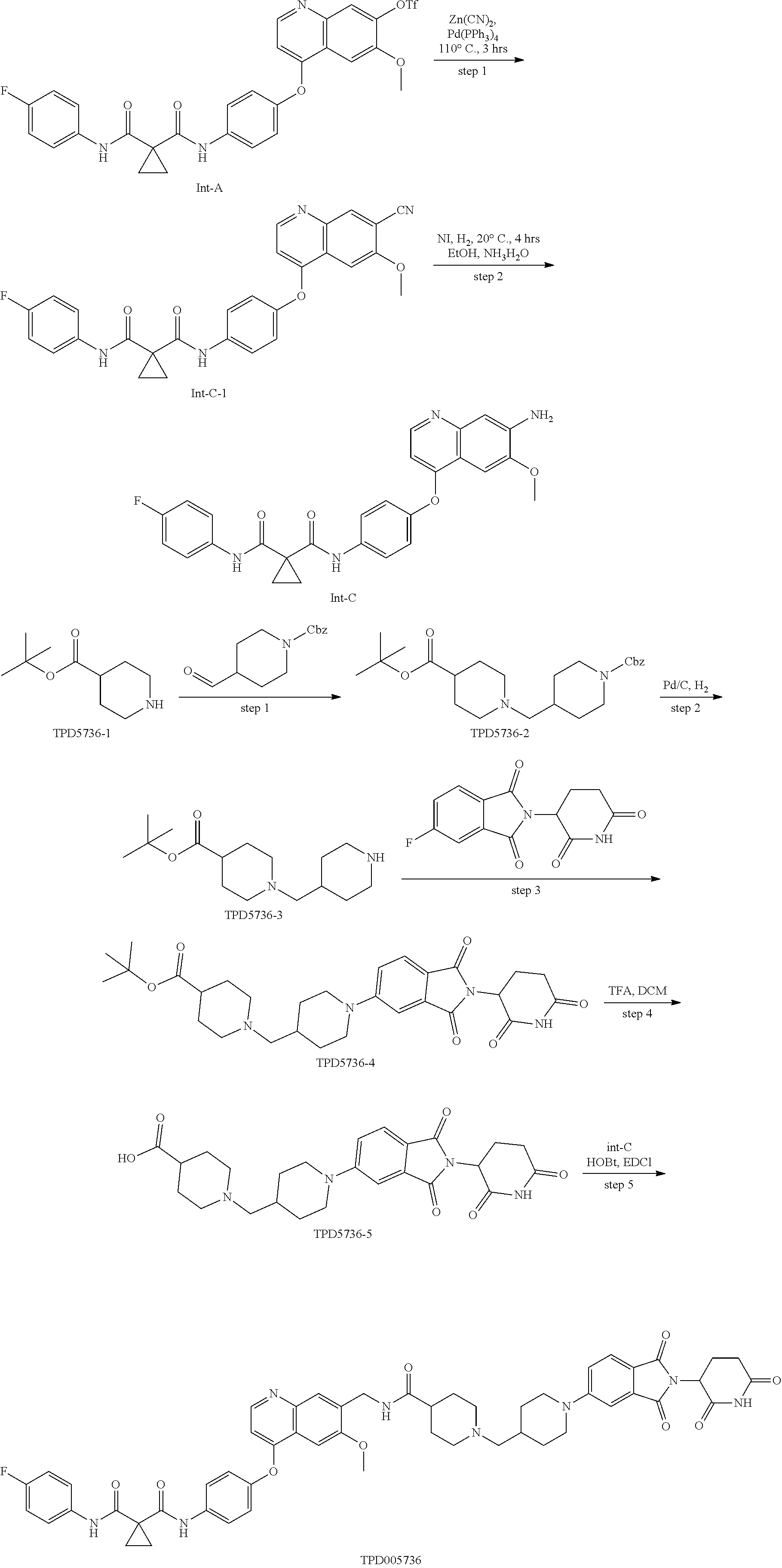

To a 100 ml single-necked vial was added the compound TPD5736-1 (5 g, 0.027 mmol), benzyl 4-formylpiperidine-1-carboxylate (6.68 g, 0.027 mmol), sodium triacetoxyborohydride (11.44 g, 0.054 mmol), and ethylene dichloride (100 ml) in this order. the reaction was carried out at 15° C. for 16 h. After completion of the reaction, the system was poured into water (50 ml) and extracted with dichloromethane (50 ml) for three times. After completion of the reaction the system was poured into water (50 ml) and extracted three times with dichloromethane (50 ml). The combined organic phases were washed with water (50 ml), saturated saline (50 ml), dried with anhydrous sodium sulfate, filtered and concentrated under reduced pressure. The residue was purified by silica gel column with eluent ratio of PE/EA=10/1˜3/1 to obtain the compound TPD5736-2 (6.2 g, colorless oil, purity 93.260), yield: 51.48%. LCMS (ESI) m/z calcd. for C₂₄H₃₆N₂O₄ [M+H]⁺ 417.3; found 417.2; ¹H NMR (400 MHz, CDCl₃): δ=7.41-7.28 (m, 5H), 5.12 (s, 2H), 4.16 (s, 2H), 2.78 (d, J=11.3 Hz, 4H), 2.24-2.07 (m, 3H), 1.94 (t, J=10.9 Hz, 2H), 1.82 (d, J=10.6 Hz, 2H). 1.75-1.63 (m, 4H), 1.60 (s, 1H), 1.43 (s, 9H), 1.08 (d, J=11.7 Hz, 2H). The compound structure is confirmed.

To a 100 ml single-necked vial was sequentially added compound TPD5736-2 (2.0 g, 4.8 mmol), THF (40 ml) and palladium charcoal (0.4 g, 10% content, 50% water content). The system reacted under 1 atm hydrogen at 15° C. for 16 hours. After completion of the reaction filtration was carried out with diatomaceous earth and washed with THF. The filtrate was concentrated under reduced pressure to give the compound TPD5736-3 (1.2 g, white solid, purity 55.619%), yield: 50.00%. LCMS (ESI) m/z calcd. for C₁₆H₃₀N₂O₂ [M+H]⁺ 283.2; found 283.1; ¹H NMR (400 MHz, CDCl₃): δ=3.06 (d, J=12.1 Hz, 2H), 2.80 (d, J=11.5 Hz, 2H), 2.58 (td, J=12.1, 2.0 Hz, 2H), 2.19-2.14 (m, 1H), 2.12 (d, J=7.1 Hz, 2H), 1.92 (t, J=11.3 Hz, 2H), 1.82 (d, J=10.0 Hz, 2H), 1.74-1.88 (m, 4H), 1.82-1.57 (m, 3.8 Hz, 1H), 1.43 (s, 8H), 1.12-1.02 (m, 2H). The compound structure is confirmed.

Compound TPD5736-3 (562.36 mg, 1.991 mmol) was added to a 40 ml single-necked vial, 2-(2,6-dioxo-piperidin-3-yl)-5-fluoroisoindole-1,3-dione (500 mg, 1.810 mmol), DIEA (701.85 mg, 5.431 mmol), and DMSO (5 ml). 100° C. reacted for 16 hours. After the reaction was completed it was cooled to room temperature and then poured into cold water (20 ml) and filtered the filter cake was purified by silica gel column with eluent ratio of PE/EA=5/1˜2/1 to obtain the compound TPD5736-4 (750 mg, yellow solid, purity 91.595%), yield: 70.46%. LCMS (ESI) m/z calcd. for C₂₉H₃₈N₄O₆ [M+H]⁺ 539.3; found 229.2 and 539.1; ¹H NMR (400 MHz, CDCl₃): δ=8.25 (s, 1H), 7.66 (d, J=8.6 Hz, 1H), 7.28 (d, J=2.2 Hz, 1H), 7.05-7.02 (m, 1H), 4.96-4.92 (m, 1H), 4.02-3.86 (m, 2H), 2.98-2.92 (m, 2H), 2.88-2.73 (m, 4H), 2.26-2.06 (m, 4H), 1.97 (t, J=10.8 Hz, 2H), 1.86 (t, J=13.1 Hz, 4H), 1.77-1.53 (m, 4H), 1.44 (s, 9H), 1.29-1.21 (m, 2H). The compound structure is confirmed.

A 50 ml single-necked vial was charged with compound TPD5736-4 (500 mg, 0.928 mmol) and HCl/EA (4N, 20 ml). the reaction was carried out at 20° C. for 16 h and then warmed up to 60° C. for another 8 h. After the reaction, the system was concentrated to dryness under reduced pressure to give compound TPD5736-5 (450 mg, yellow solid), yield: 79.45%. After completion of the reaction the system was concentrated to dryness under reduced pressure to give the compound TPD5736-5 (450 mg, yellow solid, purity 85.053%), yield: 79.45%. LCMS (ESI) m/z calcd. for C₂₅H₃₀N₄O₆ [M+H]⁺ 483.2; found 483.0. The compound structure is confirmed.

Compound TPD5736-5 (100 mg, 0.2072 mmol), int-C (103.71 mg, 0.2072 mmol), HATU (157.57 mg, 0.4144 mmol), triethylamine (41.93 mg, 0.4144 mmol), and DMF (3 ml) were added sequentially to a 25 ml single-necked vial. The reaction was carried out under nitrogen protection at 25° C. for 16 hours. After completion of the reaction, the reaction solution was poured into water (3 ml) and extracted three times with EA (1 ml). The organic phases were combined, washed with saturated brine, dried over anhydrous sodium sulfate, filtered, and concentrated to dryness. The residue was purified by preparative plate with unfolding agent ratio DCM/MeOH=10/1 to give compound TPD005736 (25 mg, yellow solid, purity 97.359%), yield: 12.16%. LCMS (ESI) m/z calcd. for C₅₃H₅₃FN₈O₉ [M+H]⁺ 965.4; found 965.3; ¹H NMR (400 MHz, DMSO_d₆): δ=11.09 (s, 1H), 10.20 (s, 1H), 10.06 (s, 1H), 8.51 (d, J=5.2 Hz, 1H), 8.40 (s, 1H), 7.79-7.64 (m, 6H), 7.54 (s, 1H), 7.31-7.13 (m, 6H), 6.52 (d, J=4.8 Hz, 1H), 5.09-5.04 (m, 1H), 4.40 (s, 2H), 4.05 (d, J=12.4 Hz, 2H), 3.98 (s, 3H), 3.00-2.88 (m, 5H), 2.14 (s, 2H), 1.99 (s, 2H), 1.90 (s, 2H), 1.79 (d, J=12.8 Hz, 4H), 1.79 (d, J=12.8 Hz, 4H), 1.68 (br. s., 2H), 1.47 (s, 4H), 1.23-1.16 (m, 5H). The compound structure is confirmed.

To a 50 ml triple-necked vial was sequentially added compound int-A (500 mg, 0.807 mmol), zinc cyanide (189.52 mg, 1.614 mmol), tetrakis(triphenylphosphine)palladium (93.25 mg, 0.0807 mmol) and N,N-dimethylformamide (10 ml). The reaction was then carried out at 110° C. for 3 hours. The reaction solution was poured into water (30 ml) and extracted three times with EA (10 ml). The organic phases were combined, washed with saturated brine, dried over anhydrous sodium sulfate, filtered, and concentrated to dryness. The residue was purified by silica gel column with eluent and ratio of PE/EtOAc=3/1˜1/1 to give compound int-C-1 (250 mg, yellow solid, purity 98.176%), yield: 61.26%. LCMS (ESI) m/z calcd. for C₂₈H₂₁FN₄O₄ [M+H]⁺ 497.15; found 497.6; ¹H NMR (400 MHz, DMSO_d₆): δ=10.23 (s, 1H), 10.49 (s, 1H), 8.66 (d, J=4.8 Hz, 1H), 8.57 (s, 1H), 7.80 (d, J=8.8 Hz, 2H), 7.77 (s, 1H), 7.66-7.63 (m, 2H), 7.28 (d, J=8.8 Hz=8.8 Hz, 2H), 7.16 (t, J=8.8 Hz, 2H), 6.66 (d, J=5.2 Hz, 1H), 4.08 (s, 3H), 1.48 (s, 4H). The compound structure is confirmed.

Compound int-C-1 (250 mg, 0.5035 mmol), ethanol (5 ml) and ammonia (3 ml) were sequentially added to a 50 ml single-necked vial. Then Raney nickel (100 mg) was added. The reaction was then carried out under a hydrogen balloon at 20° C. for 4 hours. The reaction solution was filtered and the filtrate was concentrated to dryness to give compound int-C (150 mg, white solid, purity 99.425%), yield: 59.19%. LCMS (ESI) m/z calcd. for C₂₈H₂₅FN₄O₄ [M+H]⁺ 501.19; found 501.4; ¹H NMR (400 MHz, DMSO_d₆): δ=10.18 (br. s., 1H), 10.05 (br. s., 1H), 8.51 (d, J=5.2 Hz, 1H), 7.80 (s, 1H), 7.77 (d, J=8.8 Hz, 2H), 7.66-7.63 (m, 2H), 7.50 (s, 1H), 7.23 (d, J=8.8 Hz, 2H). 7.15 (t, J=8.8 Hz, 2H), 6.50 (d, J=5.2 Hz, 1H), 3.96 (s, 3H), 3.86 (s, 2H), 1.47 (s, 4H). The compound structure is confirmed.

### Example 37: Synthesis of Compound TPD005745

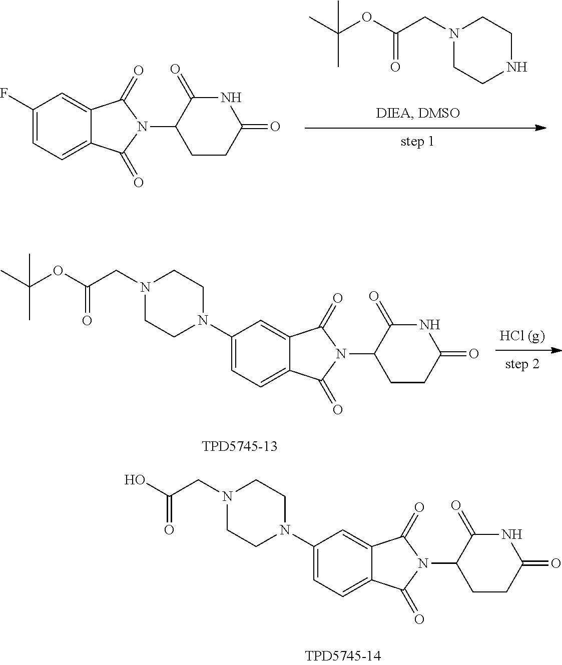 [SMILES: O=C1CCC(N2C(=O)c3ccc(F)cc3C2=O)C(=O)N1 ; CC(C)(C)OC(=O)CN1CCNCC1 ; CC(C)(C)OC(=O)CN1CCN(c2ccc3c(c2)C(=O)N(C2CCC(=O)NC2=O)C3=O)CC1 ; O=C(O)CN1CCN(c2ccc3c(c2)C(=O)N(C2CCC(=O)NC2=O)C3=O)CC1]

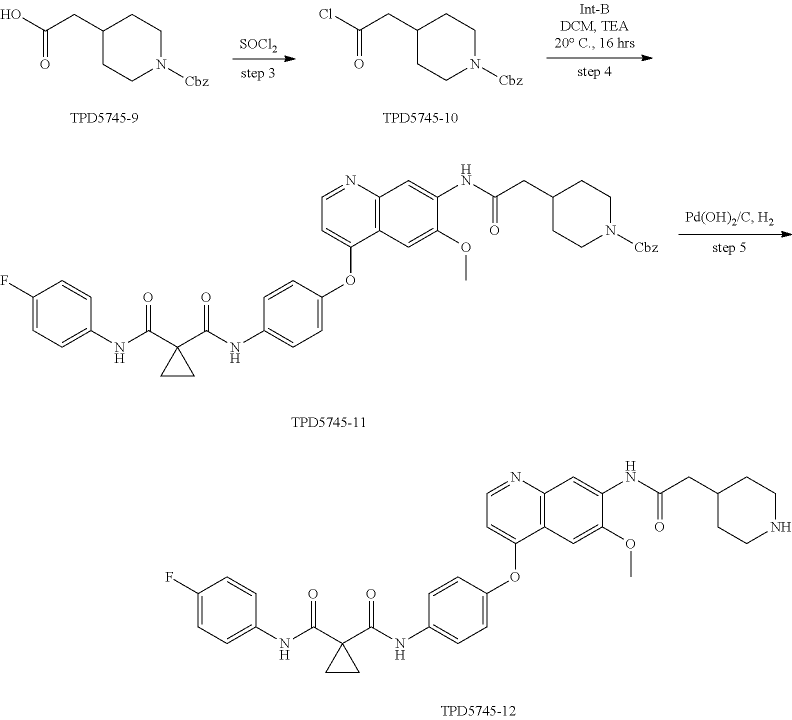 [SMILES: O=C(O)CC1CCN(C(=O)OCc2ccccc2)CC1 ; "SOCl2" ; O=C(Cl)CC1CCN(C(=O)OCc2ccccc2)CC1 ; COc1cc2c(Oc3ccc(NC(=O)C4(C(=O)Nc5ccc(F)cc5)CC4)cc3)ccnc2cc1NC(=O)CC1CCN(C(=O)OCc2ccccc2)CC1 ; COc1cc2c(Oc3ccc(NC(=O)C4(C(=O)Nc5ccc(F)cc5)CC4)cc3)ccnc2cc1NC(=O)CC1CCNCC1]

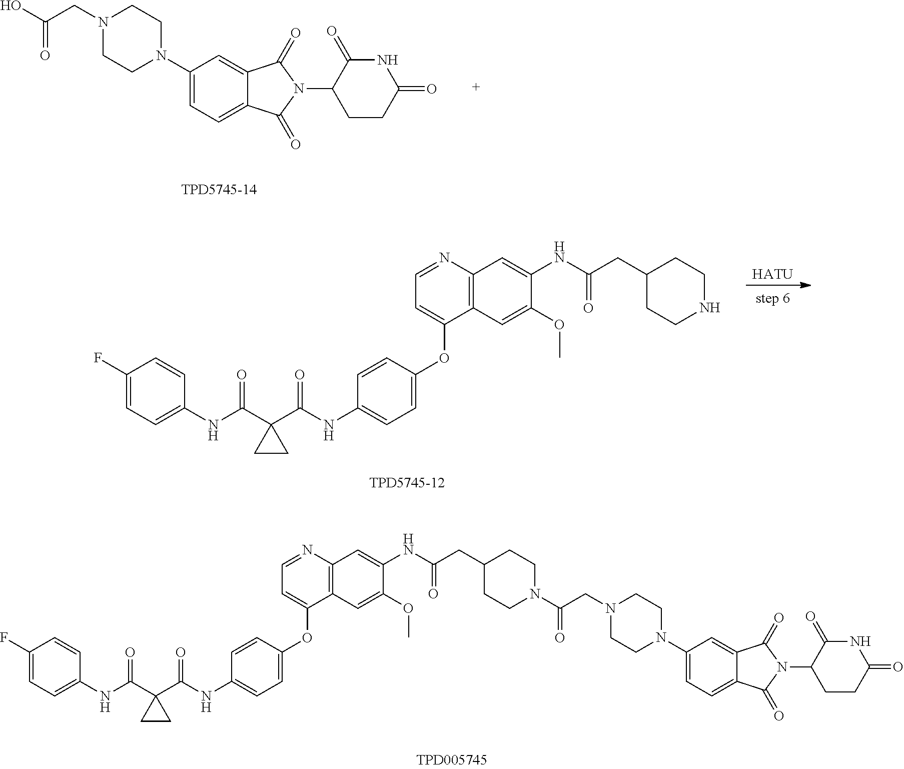

To a 50 ml triple-necked vial was sequentially added compound 2-(2,6-dioxo-piperidin-3-yl)-5-fluoroisoindole-1,3-dione (200 mg, 0.7241 mmol), tert-butyl 2-(piperidin-1-yl)acetate, DIEA (280.75 mg, 2.1723 mmol) and DMSO solution (10 ml). The reaction was carried out under nitrogen protection at 90° C. for 16 hours. After completion of the reaction, the reaction solution was reduced to room temperature, the reaction solution was poured into saturated aqueous sodium bicarbonate solution (20 ml) to precipitate a yellow solid, filtered and dissolved the yellow solid with dichloromethane, dried over anhydrous sodium sulfate and spun dry to give the compound TPD5745-13 (280 mg, yellow solid, purity 60.632%), yield: 79.50%. LCMS (ESI) m/z calcd. for C₂₃H₂₈N₄O₆ [M+H]⁺ 457.5; found 457.1. The compound structure is confirmed.

To a 50 ml triple-necked vial was sequentially added compound TPD5745-13 (200 mg, 0.4381 mmol), 3N hydrogen chloride 1,4-dioxane solution (10 ml). The reaction was carried out under nitrogen protection at 15° C. for 4 hours. After completion of the reaction, the reaction solution was concentrated to dryness to give the compound TPD5745-14 (150 mg, yellow solid, purity 92.973%), yield: 79.50%. LCMS (ESI) m/z calcd. for C₁₉H₂₀N₄O₆ [M+H]⁺ 401.14; found 401.0. The compound structure is confirmed.

A 100 ml single mouth flask was charged with sulfoxide chloride (20 ml) and compound TPD5745-9 (1 g, 0.0036 mol) was added. The reaction solution was warmed up to 70° C. for 3 hours under nitrogen protection. The reaction solution was concentrated to dryness to obtain the crude compound TPD5745-10 (0.9 g, light yellow oil, purity 93.903%), yield: 72.81%. LCMS (ESI) m/z calcd. for C₁₅H₁₈ClNO₃ [M-Cl+OCH₃+H]⁺292.35; found 292.0. The compound structure is confirmed.

A 250 ml three-necked vial was charged with int-B (450 mg, 0.925 mmol) and TEA (280.80 mg, 2.7750 mmol) in DCM (55 ml). The reaction solution was cooled down to 0° C. under nitrogen protection followed by dropwise addition of compound TPD-5745-11 (410.37 mg, 1.3875 mmol) dissolved in DCM (5 ml). After dropwise addition the reaction reacted at 15° C. for one and a half hours. The reaction solution was poured into saturated aqueous sodium bicarbonate solution (30 ml) and extracted three times with DCM (20 ml). The organic phases were combined, washed with saturated brine, dried over anhydrous sodium sulfate, filtered, and concentrated to dryness, and the residue was purified by silica gel chromatographic column with eluent ratio of DCM/MeOH=50/1˜30/1 to afford the compound TPD5745-11 (360 mg, white solid, purity 87.842%) in the yield: 45.84%. LCMS (ESI) m/z calcd. for C₄₂H₄₀FN₅O₇ [M+H]⁺ 746.29; found 764.4. The compound structure is confirmed.

To a 50 ml single-necked vial was sequentially added compound TPD5745-11 (300 mg, 0.4023 mmol) and Pd(OH)₂/C (100 mg, 10%) in ethyl acetate (5 ml). The reaction was carried out at 15° C. for 6 h. After completion of the reaction, it was concentrated to dryness to give the crude compound TPD5745-12 (70 mg, white solid, purity 30.526%), yield: 8.68%. LCMS (ESI) m/z calcd. for C₁₉H₂₅N₅O₃ [M+H]⁺ 612.67; found 612.1. The compound structure is confirmed.

Compound TPD5745-12 (70 mg, 0.1144 mmol), HATU (65.25 mg, 0.1716 mmol), compound TPD5745-14 (45.8 mg, 0.1144 mmol), and TEA (57.88 mg, 0.572 mmol) were added sequentially in a 50 ml single mouth vial. The reaction was carried out for 16 h at 15° C. under nitrogen protection. After the reaction was completed, the reaction solution was quenched with water. It was extracted three times with ethyl acetate (10 mL). The combined organic layers were washed with brine (20 mL), dried over anhydrous sodium sulfate, filtered and evaporated to give a residue. The residue was purified by preparative plate with unfolding agent ratio DCM/MeOH=10/1 to give compound TPD005745 (23.5 mg, yellow solid, purity 97.684%), yield: 22.03%. LCMS (ESI) m/z calcd. for C₅₃H₅₂FN₉O₁₀ [M+H]⁺ 994.05; found 994.4; ¹H NMR (400 MHz, DMSO_d₆): δ=11.08 (s, 1H), 10.19-10.09 (m, 1H), 10.05 (br. s., 1H), 9.41 (br. s., 1H), 8.78 (s, 1H), 8.49-8.47 (m, 1H), 7.68 (br. s., 2H), 7.64 (br. s., 3H), 7.35 (s, 1H), 7.23 (s, 1H), 7.16 (br. s., 3H), 7.13 (br. s., 2H), 6.47-6.44 (m, 1H), 5.10-5.05 (m, 1H), 4.36-4.33 (m, 1H), 4.02-4.01 (m, 3H), 3.44 (s, 3H), 3.12-3.04 (m, 4H), 2.56 (br. s., 5H), 2.08 (br. s., 4H), 1.75 (m, 2H), 1.48 (s, 3H), 1.23-0.93 (m, 7H). The compound structure is confirmed.

### Example 38: Synthesis of Compound TPD005746

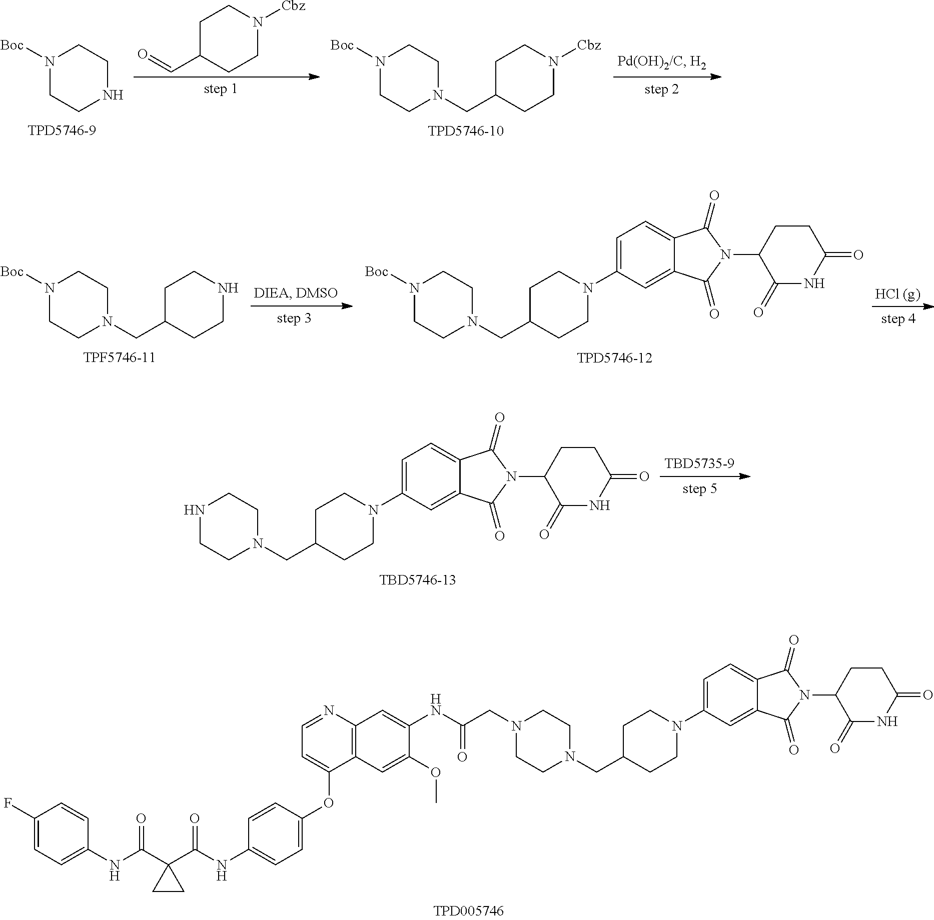

To a 250 ml single-necked vial were sequentially added 1-Boc-piperazine (5.0 g, 26.7 mmol), 1-Cbz-4-piperidinecarboxaldehyde (6.6 g, 26.7 mmol), acetic acid (4.81 g, 80.1 mmol) and sodium triacetoxyborohydride (11.32 g, 53.4 mmol). The reaction was carried out at 20° C. for 16 hours. After the reaction was completed the reaction solution was poured into saturated aqueous sodium bicarbonate solution (200 ml). It was extracted three times with DCM (100 ml). The combined organic phases were washed with saturated saline (100 ml), dried over anhydrous sodium sulfate, filtered, and the filtrate was spun dry to give a residue. The residue was purified by silica gel column with eluent and ratio of DCM/MeOH=60/1˜40/1 to obtain compound TPD5746-10 (5.1 g, colorless oil, purity 85.387%), yield: 38.95%. LCMS (ESI) m/z calcd. for C₂₃H₃₅N₃O₄ [M+H]⁺ 418.2; found 418.0; ¹H NMR (400 MHz, CDCl₃): δ=7.36-7.30 (m, 5H), 5.12 (s, 2H), 4.17 (br. s. 2H), 3.40 (t, J=4.8 Hz, 4H), 2.77 (br. s. 2H), 2.33 (t, J=4.8 Hz, 4H), 2.17-2.15 (m, 2H), 1.76-1.61 (m, 3H), 1.46 (s, 9H), 1.13-1.05 (m, 2H). The compound structure is confirmed.

A 100 ml single-necked vial was charged with compound TPD5746-10 (1 g, 2.4 mmol), EA (20 ml) and palladium carbon hydroxide (0.1 g, 10% palladium, 50% water). The reaction was carried out at 25° C. by passing 20 psi hydrogen for 2 hours. Filtration and concentration to dryness afforded compound TPD5746-11 (320 mg, gray solid, purity 43.468%), yield: 20.83%. LCMS (ESI) m/z calcd. for C₁₅H₂₉N₃O₂ [M+H]⁺ 284.2; found 284.1; ¹H NMR (400 MHz, DMSO_d₆): δ=3.28 ((t, J=5.2 Hz, 5H), 2.87 (d, J=12.0 Hz, 2H), 2.45-2.35 (m, 2H), 2.28-2.21 (m, 4H), 2.07 (d, J=7.0 Hz, 2H), 1.64-1.49 (m, 3H), 0.97-0.87 (m, 2H). The compound structure is confirmed.

A 25 mL single-necked vial was charged with compound TPD5746-11 (320 mg, 1.1251 mmol), DIEA (436.22 mg, 3.3753 mmol), DMSO (2 ml), and 2-(2,6-dioxo-piperidin-3-yl)-5-fluoroisoindole-1,3-dione (310.78 mg, 1.1251 mmol). 100° C. for 16 hours. Cooled to room temperature, the reaction solution was poured into water (20 ml) and stirred for 10 min. Filtration, the filter cake was washed with water and purified by silica gel column with eluent ratio of DCM/MeOH=100/1˜50/1 to obtain compound TPD5746-12 (510 mg, yellow solid, purity 97.998%), yield: 82.17%. LCMS (ESI) m/z calcd. for C₂₈H₃₇N₅O₆ [M+H]⁺ 540.3; found 540.1 and 484.1.

A 25 mL single-necked vial was charged with compound TPD5746-12 (260 mg, 0.4809 mmol) and hydrogen chloride dioxane solution (3 N, 10 ml). the reaction was carried out at 25° C. for 2 h. The reaction solution was concentrated to dryness to give compound TPD5746-13 (248 mg, yellow solid, purity: 95.127%). The reaction solution was concentrated to dryness to give compound TPD5746-13 (248 mg, yellow solid, purity 95.127%), yield: 95.74%. LCMS (ESI) m/z calcd. for C₂₃H₂₉N₅O₄ [M+H]⁺ 440.22; found 440.1. The compound structure is confirmed.

To a 25 ml single-necked vial was sequentially added compound TPD5746-13 (43 mg, 0.0839 mmol), compound TPD5735-9 (47.23 mg, 0.0839 mmol), potassium carbonate (34.79 mg, 0.0839 mmol), acetonitrile (6 ml) and potassium iodide (1.39 mg, 0.00839 mmol). the reaction was carried out at 25° C. for 16 hours. The reaction solution was poured into water (10 ml), EA (10 ml) was extracted 3 times, the combined organic phases were washed with water, brine, dried with anhydrous sodium sulfate filtered and concentrated and purified by preparative plates, the ratio of unfolding agent was DCM/MeOH=10/1 to give compound TPD005746 (31.4 mg, yellow solid, purity 97.503%) in 37.78% yield. LCMS (ESI) m/z calcd. for C₅₂H₅₂FN₉O₉ [M+H]⁺ 966.4; found 966.4; ¹H NMR (400 MHz, DMSO_d₆): δ=11.10 (s, 1H), 10.22 (d, J=4.9 Hz, 2H), 10.07 (s, 1H), 8.88 (s, 1H), 8.52 (t, J=5.3 Hz, 1H), 7.79 (d, J=8.9 Hz, 2H), 7.69-7.62 (m, 4H), 7.33 (s, 1H), 7.26 (d, J=9.0 Hz, 3H), 4.12 (t, J=8.9 Hz, 2H), 6.49 (d, J=5.1 Hz, 1H), 5.11-5.06 (m 1H), 4.11-4.05 (d, J=19.4 Hz, 5H), 3.25 (s, 2H), 3.07-2.83 (m, 4H), 2.73-2.59 (m, 6H), 2.23 (d, J=6.1 Hz, 2H), 2.10-1.92 (m, 2H), 1.83 (d, J=12.6 Hz, 2H). 1.49 (s, 4H), 1.26-1.16 (m, 5H). The compound structure is confirmed.

### Example 39: Synthesis of Compound TPD005752m

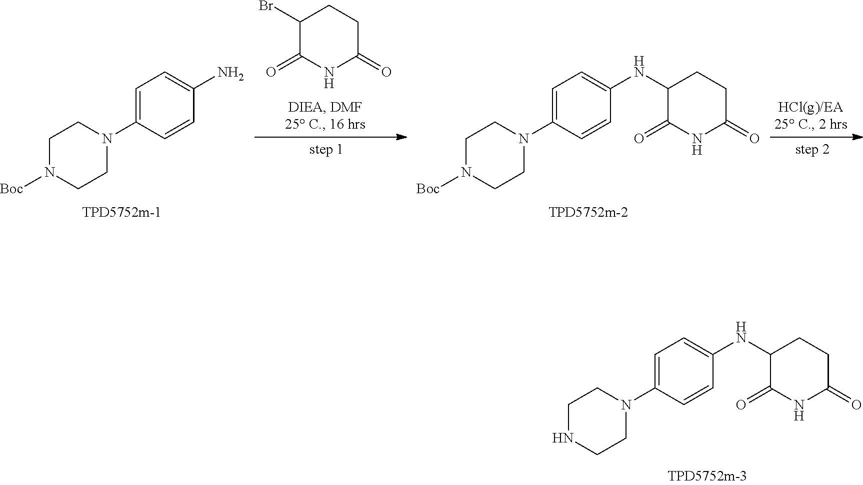 [SMILES: CC(C)(C)OC(=O)N1CCN(c2ccc(N)cc2)CC1 ; O=C1CCC(Br)C(=O)N1 ; CC(C)(C)OC(=O)N1CCN(c2ccc(NC3CCC(=O)NC3=O)cc2)CC1 ; O=C1CCC(Nc2ccc(N3CCNCC3)cc2)C(=O)N1]

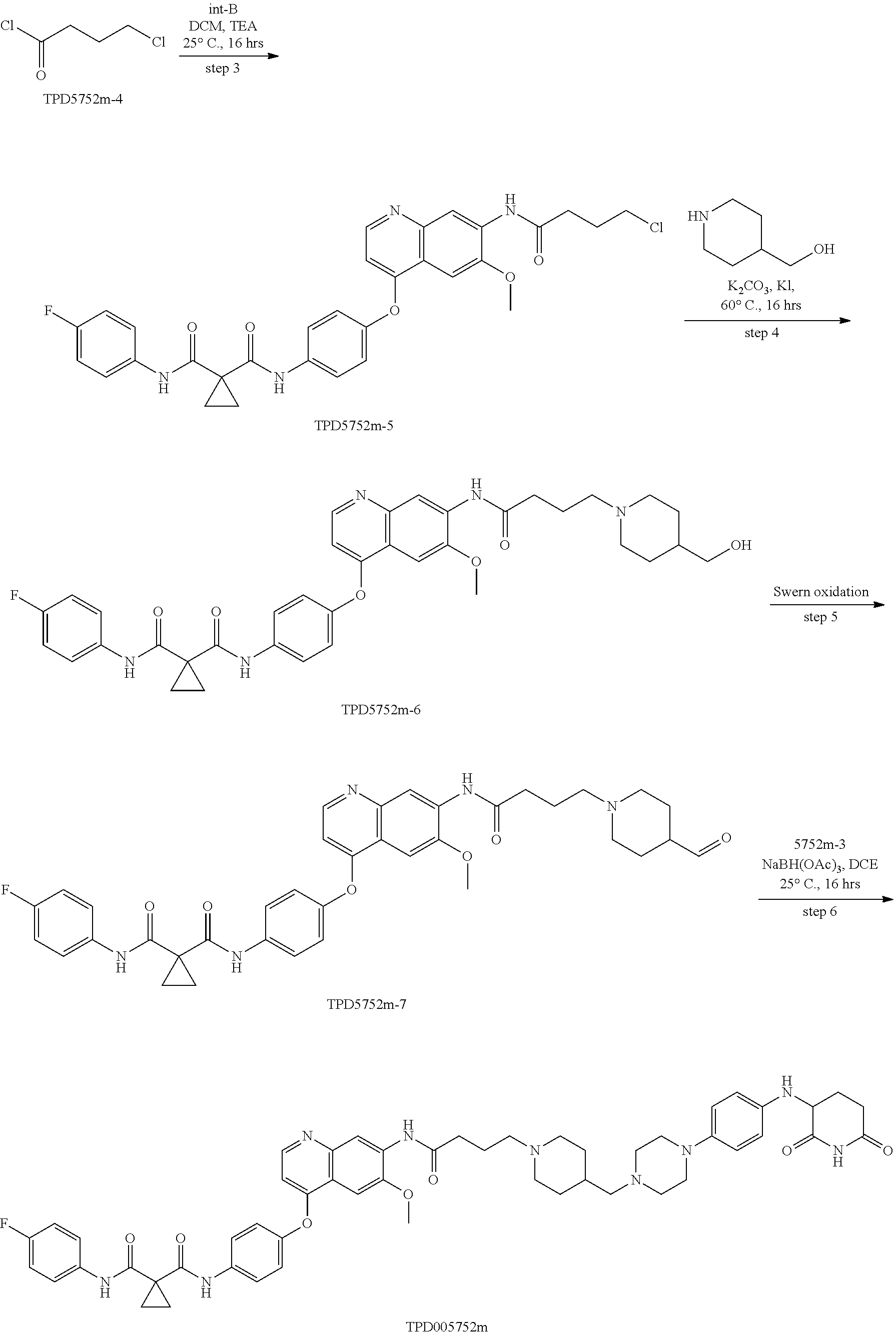 [SMILES: O=C(Cl)CCCCl ; COc1cc2c(Oc3ccc(NC(=O)C4(C(=O)Nc5ccc(F)cc5)CC4)cc3)ccnc2cc1NC(=O)CCCCl ; OCC1CCNCC1 ; COc1cc2c(Oc3ccc(NC(=O)C4(C(=O)Nc5ccc(F)cc5)CC4)cc3)ccnc2cc1NC(=O)CCCN1CCC(CO)CC1 ; COc1cc2c(Oc3ccc(NC(=O)C4(C(=O)Nc5ccc(F)cc5)CC4)cc3)ccnc2cc1NC(=O)CCCN1CCC(C=O)CC1 ; COc1cc2c(Oc3ccc(NC(=O)C4(C(=O)Nc5ccc(F)cc5)CC4)cc3)ccnc2cc1NC(=O)CCCN1CCC(CN2CCN(c3ccc(NC4CCC(=O)NC4=O)cc3)CC2)CC1]

To a 100 ml triple-necked vial was sequentially added the compound tert-butyl 4-(4-aminophenyl)piperazine-1-carboxylate (2 g, 7.2 mmol), 3-bromopiperidine-2,6-dione (2.76 g, 14.4 mmol), DIEA (1.86 g, 14.4 mmol) and DMF (20 ml). The reaction was carried out under nitrogen protection at 25° C. for 16 hours. After completion of the reaction, the reaction solution was poured into water (30 ml) and extracted three times with EA (10 ml). The organic phases were combined, washed with saturated brine, dried over anhydrous sodium sulfate, filtered, and concentrated to dryness, and the residue was purified by silica gel chromatographic column with eluent ratio of DCM/MeOH=100/1˜30/1 to afford the compound TPD5752m-2 (1.1 g, green solid, purity 95.775%) in the yield: 37.50%. LCMS (ESI) m/z calcd. for C₂₀H₂₈N₄O₄ [M+H]⁺ 389.2; found 389.1; ¹H NMR (400 MHz, DMSO_d₆): δ=10.77 (s, 1H), 6.78-6.76 (m, 2H), 6.62-6.60 (m, 2H), 5.45 (d, J=7.4 Hz, 1H), 4.24-4.18 (m, 1H), 3.43 (br. s., 4H), 2.87-2.85 (m, 4H), 2.77-2.68 (m, 1H), 2.60-2.54 (m, 1H), 2.12-2.08 (m, 1H), 1.88-1.78 (m, 1H), 1.41 (s, 9H). The compound structure is confirmed.

To a 25 ml single-necked vial was sequentially added compound TPD5752m-2 (250 mg, 0.6419 mmol), EA (1 ml) and HCl/EA (2 ml). The reaction was carried out at 25° C. for 2 h. After completion of the reaction, it was concentrated to dryness to give the crude compound TPD5752m-3 (250 mg, blue solid), yield: 75.14%. LCMS (ESI) m/z caled. for C₁₅H₂₀N₄O₂ [M+H]⁺ 289.2; found 289.2. The compound structure is confirmed.

To a 100 ml triple-necked vial were sequentially added compound int-B (862.6 mg, 1.7731 mmol), TEA (717.68 mg, 7.0924 mmol) and DCM (10 ml). After cooling down to 0° C. compound TPD5752m-4 (500 mg. 3.5462 mmol). The reaction was carried out under nitrogen protection at 25° C. for 16 hours. After completion of the reaction, the reaction solution was lowered to 0° C. and decanted into water (30 ml) and extracted three times with DCM (10 ml). The organic phases were combined, washed with saturated brine, dried over anhydrous sodium sulfate, filtered, and concentrated to dryness to afford the crude compound TPD5752m-5 (480 mg, brown oil, purity 76.441%), yield: 17.51%. LCMS (ESI) m/z calcd. for C₃₁H₂₈ClFN₄O₅ [M+H]⁺ 591.2; found 591.2; ¹H NMR (400 MHz, DMSO_d₆): δ=10.20 (s, 1H), 10.07 (s, 1H), 9.54 (s, 1H), 8.78 (s, 1H), 8.50 (d, J=5.2 Hz, 1H), 7.78 (d, J=8.9 Hz, 2H), 7.67-7.62 (m, 3H), 7.26-7.24 (m, 2H), 7.19-7.13 (m, 3H), 4.03 (s, 3H), 3.73 (t, J=6.6 Hz, 2H), 2.70 (t, J=7.3 Hz, 2H), 2.10-2.06 (m, 2H), 1.48 (s, 4H). The compound structure is confirmed.

A 50 ml triple-necked vial was sequentially charged with compounds TPD5752m-5 (500 mg, 0.846 mmol), 4-hydroxymethylpiperidine (194.75 mg, 1.692 mmol), K₂CO₃ (233.85 mg, 1.692 mmol), KI (28.09 mg, 0.1692 mmol) and acetonitrile (10 ml). The reaction was carried out under nitrogen protection at 60° C. for 16 hours. After the reaction was completed, the reaction solution was reduced to room temperature and poured into water (10 ml) and extracted three times with EA (3 ml). The organic phases were combined, washed with saturated brine, dried over anhydrous sodium sulfate, filtered, and concentrated to dryness, and the residue was purified by silica gel chromatographic column with eluent ratio of DCM/MeOH=100/1˜30/1 to afford the compound TPD5752m-6 (240 mg, brown solid, purity 97.264%) in the yield: 41.19%. LCMS (ESI) m/z calcd. for C₃₇H₄₀FN₅O₆ [M+H]⁺ 670.3; found 670.3; ¹H NMR (400 MHz, DMSO_d₆): δ=10.20 (s, 1H), 10.06 (s, 1H), 9.39 (s, 1H), 8.79 (s, 1H), 8.49 (d, J=5.2 Hz, 1H), 7.77 (d J=8.9 Hz, 1H), 7.66-7.63 (m, 3H), 7.27-7.23 (m, 2H), 7.18-7.13 (m, 3H), 4.44 (s, 1H), 4.03 (s, 2H), 3.22-3.17 (m, 4H), 2.97 (br. s., 2H), 2.55-2.54 (m, 2H). 1.82 (br. s., 2H), 1.67-1.64 (m, 3H), 1.48 (s, 3H), 1.36-1.34 (m, 2H), 1.17 (br. s., 2H). The compound structure is confirmed.

Compound oxalyl chloride (90.96 mg, 0.7166 mmol) and DCM (2 ml) were sequentially added to a 50 ml triple-necked vial, cooled down to −78° C. A DCM (1 ml) solution of DMSO (111.79 mg, 1.4332 mmol) was added and −78° C. stirred for 0.5 hours. Then a DCM (2 ml) solution of compound TPD5752m-6 (240 mg, 0.3583 mmol) was added and −78° C. stirred for 0.5 hours. Finally, TEA (290.05 mg, 2.8664 mmol) was added and the reaction was slowly restored to room temperature and protected by nitrogen for 1 hour. After completion of the reaction, the reaction solution was poured into saturated NaHCO₃ solution (6 ml) and extracted three times with DCM (3 ml). The organic phases were combined, washed with saturated brine, dried over anhydrous sodium sulfate, filtered, and concentrated to dryness to give the crude compound TPD5752m-7 (240 mg, brown solid, purity 31.626%), yield: 31.73%. LCMS (ESI) m/z calcd. for C₃₇H₃₈FN₅O₆ [M+H]⁺ 668.3; found 668.3. The compound structure is confirmed.

To a 25 ml single-necked vial was sequentially added compound TPD5752m-7 (160 mg, 0.2396 mmol), compound TPD5752m-3 (82.91 mg, 0.2875 mmol), sodium triacetoxyborohydride (101.56 mg, 0.4792 mmol) and DCE (3 ml). The reaction was carried out under nitrogen protection at 25° C. for 16 hours. The reaction solution was decanted into water (5 ml) and appropriate amount of saturated NaHCO₃ solution was added to make the reaction solution alkaline. The organic phase was separated and the aqueous phase was extracted twice with DCM (5 ml). The organic phases were combined, washed with saturated brine, dried over anhydrous sodium sulfate, filtered, and concentrated to dryness. The residue was prepared by high performance liquid chromatography (HPLC) with the following parameters: column: XBridge-1 5 μm 19-150 mm; mobile phase: acetonitrile-water (0.1% NH H₃₂ O); gradient: 40-80/8 min to afford compound TPD005752m (2.47 mg, gray solid, purity 87.474%), yield: 0.96%. LCMS(ESI) m/z calcd. for C₅₂H₅₈FN₉O₇ [M+H]⁺ 940.4; found 314.3 and 470.8 (one-third peaks and half peaks); ¹H NMR (400 MHz, CD₃ OD): δ=8.93 (s, 1H), 8.43 (d, J=5.4 Hz, 1H), 7.73-7.70 (m, 3H), 7.58-7.54 (m, 2H), 7.23-7.21 (m, 2H), 7.09-7.05 (m, 2H), 6.89-6.87 (m, 2H), 6.73-6.71 (m, 2H), 6.56 (d, J=5.3 Hz, 1H), 4.21-4.17 (m, 1H), 4.09 (s, 3H), 3.13-3.11 (m, 2H), 3.02 (br. s., 3H), 2.79-2.71 (m, 2H), 2.61-2.56 (m, 7H), 2.34-2.30 (m, 1H), 2.20 (t, J=6.7 Hz, 1H), 2.03-1.97 (m, 3H), 1.92-1.88 (m, 1H), 1.85-1.81 (m, 2H), 1.64 (br. s., 3H), 1.30 (s, 9H). The compound structure is confirmed.

### Example 40: Synthesis of Compound TPD005760

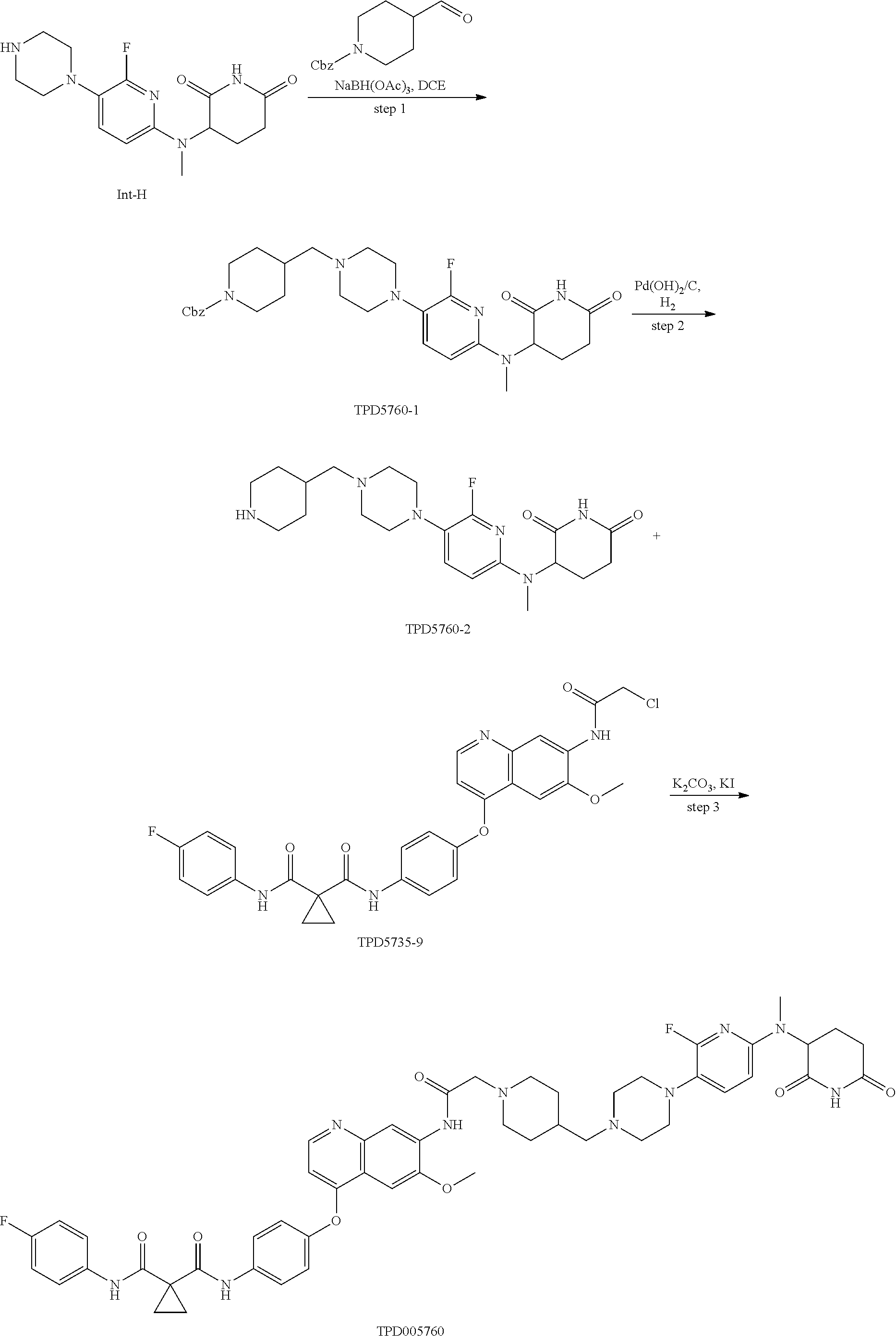

A 50 ml single-necked vial was charged with int-H (280 mg, 0.87 mmol), benzyl 4-formylpiperidine-1-carboxylate (323 mg, 1.31 mmol), DCE (10 ml), sodium triacetoxyborohydride (554 mg, 2.61 mmol) and glacial acetic acid (157 mg, 2.61 mmol). The reaction was carried out at 25° C. for 16 hours. The reaction solution was poured into saturated aqueous sodium bicarbonate solution (10 ml) and extracted three times with dichloromethane (5 ml). The organic phases were combined and washed sequentially with water (10 ml) and saturated saline (10 ml). Afterwards, it was dried with anhydrous sodium sulfate, filtered and concentrated to dryness to obtain the crude product, which was purified by silica gel column with eluent ratio of PE/EA=5/1˜0/1 to obtain the compound TPD5760-1 (160 mg, white solid, purity 92.58%), yield: 30.76%. LCMS (ESI) m/z calcd. for C₂₉H₃₇FN₆O₄ [M+H]⁺ 553.3; found 553.1; ¹H NMR (400 MHz, CDCl₃): δ=7.90 (s, 1H), 7.37-7.28 (m, 6H), 6.36 (d, J=8.4 Hz, 1H), 5.51-5.47 (m, 1H), 5.13 (s, 2H), 4.18 (br. s., 2H). 2.96 (d, J=4.2 Hz, 4H), 2.91-2.85 (m, 3H), 2.84-2.72 (m, 4H), 2.56 (s, 4H), 2.40-2.29 (m, 1H), 2.23 (d, J=7.0 Hz, 2H), 1.83-1.63 (m, 4H), 1.11 (d, J=10.8 Hz 2H). The compound structure is confirmed.

A 250 ml single mouth flask was charged with compound TPD5760-1 (160 mg, 0.35 mmol), EA (4 ml) and palladium carbon hydroxide (40 mg, 10%). The reaction was carried out at 25° C. bypassing 1 atm hydrogen for 1 hour. Filtration and concentration to dryness gave the crude compound TPD5760-2 (120 mg, brown oil, purity 39.10%), yield: 41.56%. LCMS (ESI) m/z calcd. for C₂₁H₃₁FN₆O₂ [M+H]⁺ 419.3; found 419.1. The compound structure is confirmed.

A 25 mL single-necked vial was charged with compound TPD5760-2 (55 mg, 0.13 mmol), compound TPD5735-9 (73.98 mg, 0.13 mmol), acetonitrile (5 ml), potassium carbonate (36.32 mg, 0.26 mmol), and potassium iodide (2 mg, 0.01 mmol). the reaction was carried out for 16 h at 25° C. The reaction was carried out at 25 C for 16 hours. The reaction solution was poured into water (20 ml) and extracted three times with ethyl acetate (10 ml), the combined organic phases were washed sequentially with water (20 ml) and saturated saline (20 ml), and then dried with anhydrous sodium sulfate. After filtration, purification was carried out by preparative plates with the unfolding agent ratio of DCM/MeOH=10/1 to afford the compound TPD005760 (12.6 mg, off-white solid, purity 99.553%) in a yield of 8.56%. LCMS (ESI) m/z calcd. for C₅₀H₅₄F₂N₁₀O₇ [M+H]⁺ 945.4; found 945.4; ¹H NMR (400 MHz, DMSO_d₆): δ=10.81 (s, 1H), 10.23 (d, J=24.7 Hz, 2H), 10.06 (s, 1H), 8.86 (s, 1H), 8.51 (d, J=5.2 Hz, 1H), 7.78 (d, J=8.9 Hz, 2H), 7.68-7.59 (m, 3H), 7.47-7.42 (m, 1H), 7.25 (d, J=9.0 Hz, 2H), 7.19-7.11 (m, 2H), 6.50-6.46 (m, 2H), 5.20 (d, J=7.5 Hz, 1H), 4.09 (s, 3H), 3.20 (s, 2H), 2.87 (d, J=14.8 Hz, 6H). 2.80 (s, 3H), 2.56 (s, 2H), 2.45-2.32 (m, 2H), 2.26 (t, J=9.1 Hz, 4H), 1.86-1.79 (m, 3H), 1.59 (br. s., 1H), 1.48 (s, 4H), 1.24 (d, J=10.4 Hz, 4H). The compound structure is confirmed.

### Example 41: Synthesis of Compound TPD005773

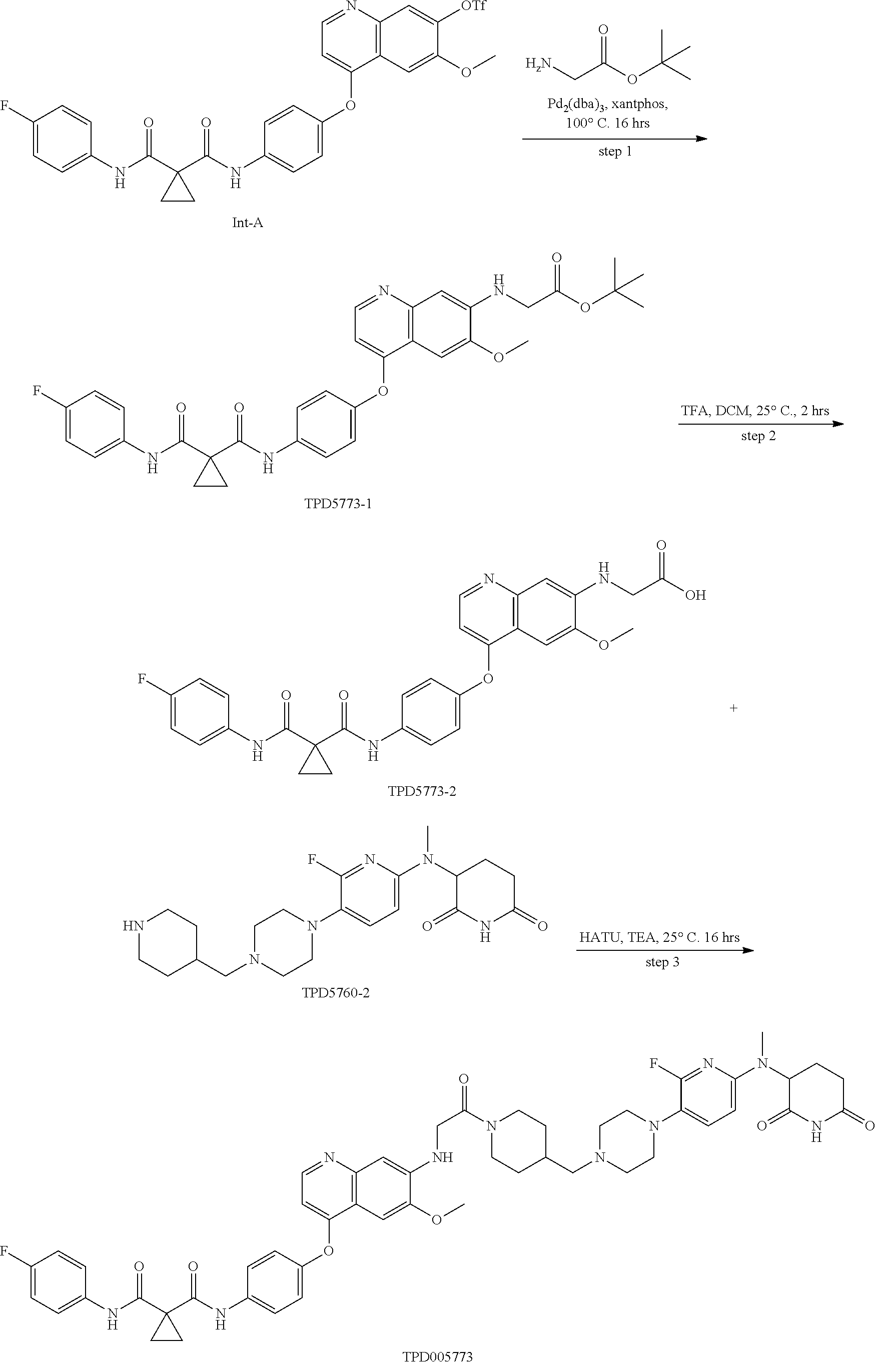

To a 250 ml triple-necked vial was sequentially added compound int-A (1.0 g, 1.61 mmol), tert-butylglycinate (423 mg, 3.23 mmol), cesium carbonate (1051 mg, 3.23 mmol), XantPhos (187 mg, 0.323 mmol), Pd₂ (dba)₃ (148 mg, 0.16 mmol) and 1,4-dioxane (50 ml). The reaction was carried out at 100° C. for 16 h under nitrogen protection. After the reaction was completed, the reaction solution was reduced to room temperature and concentrated to dryness, and the residue was purified by silica gel column with eluent ratio of DCM/MeOH=100/1˜30/1 to afford compound TPD5773-1 (680 mg, yellow solid, purity 98.573%), yield: 69.14%. LCMS (ESI) m/z calcd. for C₃₃H₃₃FN₄O₆ [M+H]⁺ 601.2; found 601.2; ¹H NMR (400 MHz, DMSO_d₆): δ=10.16 (s, 1H), 10.06 (s, 1H), 8.34 (d, J=5.2 Hz, 1H), 7.74 (d, J=8.9 Hz, 2H), 7.66-7.63 (m, 2H), 7.39 (s 1H), 7.20-7.13 (m, 4H), 6.73 (s, 1H), 6.28 (d, J=5.2 Hz, 1H), 6.08 (t, J=6.1 Hz, 1H), 3.99-3.97 (m, 5H), 1.47 (s, 4H), 1.44 (s, 9H). The compound structure is confirmed.

To a 50 ml single-necked vial was sequentially added compound TPD5773-1 (250 mg, 0.416 mmol), DCM (2 ml) and trifluoroacetic acid (1 ml). The reaction was carried out at 25° C. for 2 h. After completion of the reaction, it was concentrated to dryness to give the crude compound TPD5773-2 (250 mg, yellow solid), yield: 89.33%. LCMS (ESI) m/z calcd. for C₂₉H₂₅FN₄O₆ [M+H]⁺ 545.2; found 545.2. The compound structure is confirmed.

To a 25 ml single-necked vial was sequentially added compound TPD5773-2 (100 mg, 0.184 mmol), compound TPD5760-2 (77 mg, 0.184 mmol), HATU (105 mg, 0.275 mmol), triethylamine (37 mg, 0.367 mmol) and DMF (3 ml). The reaction was carried out under nitrogen protection at 25° C. for 16 hours. After the reaction was completed, the reaction solution was poured into water (6 ml) and extracted three times with EA (2 ml). The organic phases were combined, washed with saturated brine, dried over anhydrous sodium sulfate, filtered, and concentrated to dryness. The residue was purified by preparative plate with unfolding agent ratio DCM/MeOH=10/1 to give compound TPD005773 (26.4 mg, off-white solid, purity 96.12%), yield: 14.65%. LCMS (ESI) m/z calcd. for C₅₀H₅₄F₂N₁₀O₇ [M+H]⁺ 945.4; found 945.4; ¹H NMR (400 MHz, DMSO_d₆): δ=10.81 (s, 1H), 10.17 (s, 1H), 10.06 (s, 1H), 8.36 (d, J=5.3 Hz, 1H), 7.75 (d, J=8.9 Hz, 2H), 7.66-7.63 (m, 2H), 7.48-7.43 (m, 1H), 7.40 (s, 1H), 7.21-7.13 (m, 4H), 6.97 (s, 1H), 6.50 (d, J=8.3 Hz, 1H), 6.27 (d, J=5.3 Hz, 1H), 5.98 (br. s., 1H), 5.20 (d, J=9.6 Hz, 1H), 4.41 (d, J=13.9 Hz, 1H), 4.14-4.07 (m, 2H), 4.00 (br. s., 4H), 3.06 (t, J=12.0 Hz, 1H), 2.89-2.84 (m, 5H), 2.81 (s, 3H), 2.69 (t, J=11.4 Hz, 1H), 2.56 (br. s., 1H), 2.43-2.30 (m, 2H), 2.21 (s, 2H), 2.04-1.73 (m, 5H), 1.47 (s, 4H), 1.23 (s, 2H), 1.14 (d, J=12.6 Hz, 1H), 1.02-0.95 (m, 1H). The compound structure is confirmed.

### Example 42: Synthesis of Compound TPD005774

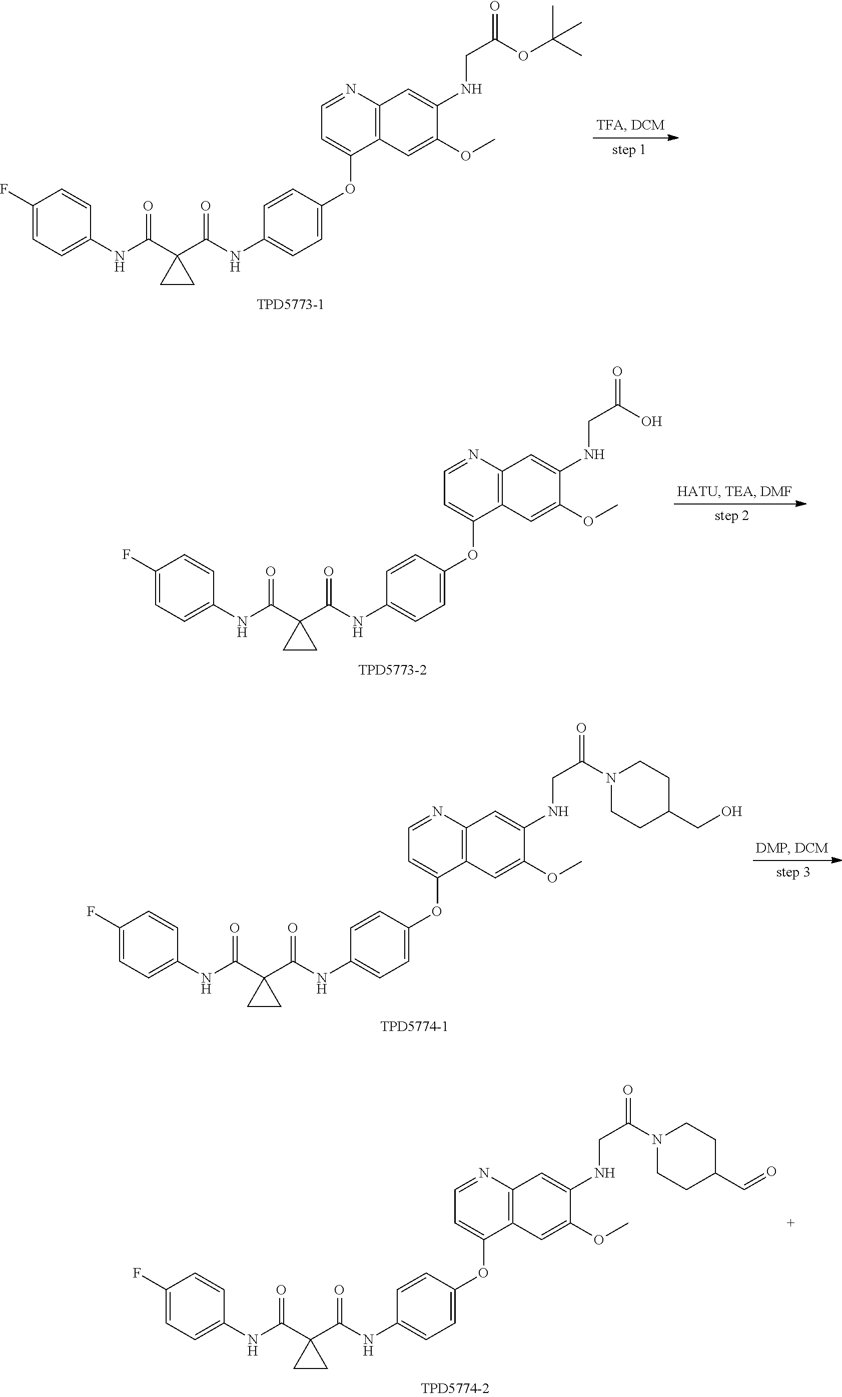 [SMILES: COc1cc2c(Oc3ccc(NC(=O)C4(C(=O)Nc5ccc(F)cc5)CC4)cc3)ccnc2cc1NCC(=O)OC(C)(C)C ; COc1cc2c(Oc3ccc(NC(=O)C4(C(=O)Nc5ccc(F)cc5)CC4)cc3)ccnc2cc1NCC(=O)O ; COc1cc2c(Oc3ccc(NC(=O)C4(C(=O)Nc5ccc(F)cc5)CC4)cc3)ccnc2cc1NCC(=O)N1CCC(CO)CC1 ; COc1cc2c(Oc3ccc(NC(=O)C4(C(=O)Nc5ccc(F)cc5)CC4)cc3)ccnc2cc1NCC(=O)N1CCC(C=O)CC1]

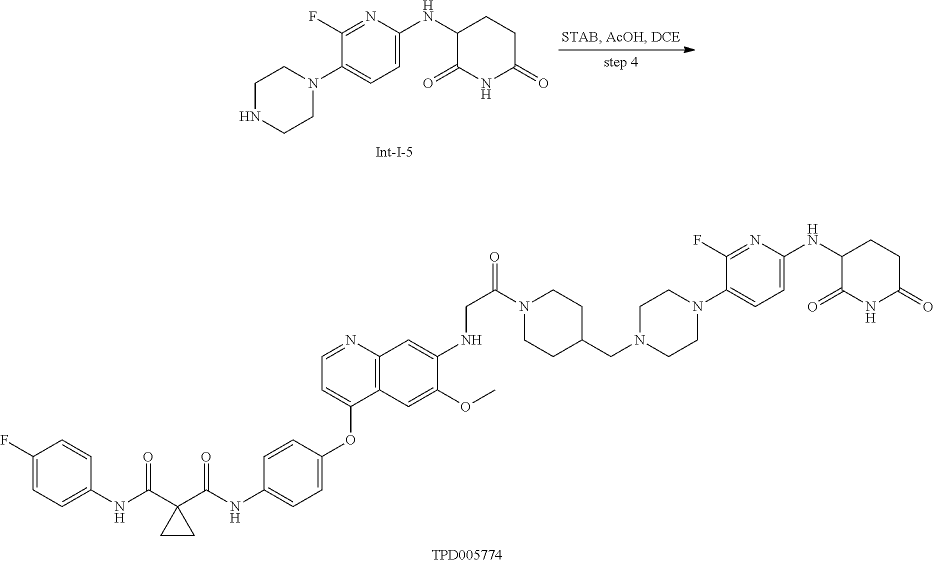 [SMILES: O=C1CCC(Nc2ccc(N3CCNCC3)c(F)n2)C(=O)N1 ; COc1cc2c(Oc3ccc(NC(=O)C4(C(=O)Nc5ccc(F)cc5)CC4)cc3)ccnc2cc1NCC(=O)N1CCC(CN2CCN(c3ccc(NC4CCC(=O)NC4=O)nc3F)CC2)CC1]

To a 50 ml single-necked vial was sequentially added compound TPD5773-1 (400 mg, 0.67 mmol), DCM (2 ml) and trifluoroacetic acid (1 ml). The reaction was carried out at 25° C. for 2 h. After completion of the reaction, it was concentrated to dryness to give the crude compound TPD5773-2 (400 mg, yellow solid), yield: 72.60%. LCMS (ESI) m/z calcd. for C₂₉H₂₅FN₄O₆ [M+H]⁺ 545.2; found 545.0. The compound structure is confirmed.

A 25 ml single-necked vial was sequentially charged with compounds TPD5773-2 (350 mg, 0.64 mmol), 4-hydroxymethylpiperidine (111 mg, 0.964 mmol), EDCI (185 mg, 0.96 mmol), HOBT (130 mg, 0.96 mmol), triethylamine (195 mg, 1.93 mmol) and DCM (5 ml). The reaction was carried out under nitrogen protection at 25° C. for 16 hours. After completion of the reaction, the reaction solution was poured into water (6 ml) and extracted three times with DCM (2 ml). The organic phases were combined, washed with saturated brine, dried over anhydrous sodium sulfate, filtered, and concentrated to dryness. The residue was purified by silica gel column with eluent ratio of DCM/MeOH=100/1˜30/1 to give compound TPD5774-1 (240 mg, yellow solid, purity 96.292%), yield: 56.02%. LCMS (ESI) m/z calcd. for C₃₅H₃₆FN₅O₆ [M+H]⁺ 642.3; found 642.1; ¹H NMR (400 MHz, DMSO_d₆): δ=10.17 (s, 1H), 10.07 (s, 1H), 8.35 (d, J=5.2 Hz, 1H), 7.74 (d, J=8.9 Hz, 2H), 7.66-7.63 (m, 2H), 7.39 (s, 1H), 7.21-7.13 (m, 4H), 6.97 (s, 1H), 6.27 (d, J=5.2 Hz, 1H), 5.96 (t, J=4.2 Hz, 1H), 4.54 (t, J=5.2 Hz, 1H), 4.44-4.36 (m, 1H), 4.13-4.04 (m, 3H), 4.00 (s, 3H), 3.27 (t, J=5.6 Hz 3H), 3.17 (d, J=4.7 Hz, 1H), 3.03 (t, J=11.9 Hz, 1H), 2.65 (t, J=11.7 Hz, 1H), 1.77-1.65 (m, 3H), 1.47 (s, 4H). The compound structure is confirmed.

Compound TPD5774-1 (190 mg, 0.30 mmol) and DCM (5 ml) were added sequentially in a 25 ml triple-necked vial. After cooling down to 0° C., Dace-Martin oxidizer (251 mg, 0.59 mmol) was added. The reaction was carried out for 3 hours at 25° C. under nitrogen protection. After completion of the reaction, saturated NaHCO₃ solution (3 ml) and water (5 ml) were added to the reaction solution and extracted three times with DCM (3 ml). The organic phases were combined, washed with saturated brine, dried over anhydrous sodium sulfate, filtered, and concentrated to dryness, and the residue was purified by preparative plate with unfolding reagent ratio of DCM/MeOH=10/1 to afford the compound TPD5774-2 (90 mg, white solid, purity 84.277%), yield: 40.05%. LCMS (ESI) m/z calcd. for C₃₅H₃₄FN₅O₆ [M+H]⁺ 640.2; found 640.1; ¹H NMR (400 MHz, DMSO_d₆): δ=10.18 (s, 1H), 10.07 (s, 1H), 9.62 (s, 1H), 8.35 (d, J=5.2 Hz, 1H), 7.74 (d, J=8.9 Hz, 2H), 7.66-7.62 (m, 2H), 7.39 (s, 1H), 7.21-7.13 (m, 4H), 6.97 (s, 1H), 6.27 (d, J=5.2 Hz, 1H), 5.95 (t, J=4.3 Hz, 1H), 4.20-4.09 (m, 3H), 4.00 (s, 3H), 3.92 (d, J=13.7 Hz 1H), 3.27-3.16 (m, 2H), 3.01-2.92 (m, 1H), 2.68-2.60 (m, 1H), 1.99-1.87 (m, 2H), 1.60-1.53 (m, 1H), 1.47 (s, 4H). The compound structure is confirmed.

Compound TPD5774-2 (90 mg, 0.14 mmol), Int-1-5 (86 mg, 0.28 mmol), sodium triacetoxyborohydride (60 mg, 0.28 mmol), acetic acid (25 mg, 0.42 mmol), and DCE (3 ml) were added sequentially to a 25 ml single-necked vial. The reaction was carried out under nitrogen protection at 30° C. for 16 hours. The reaction solution was poured into water (5 ml) and an appropriate amount of saturated NaHCO₃ solution was added to make the reaction solution alkaline. The organic phase was separated and the aqueous phase was extracted twice with DCM (5 ml). The organic phases were combined, washed with saturated brine, dried over anhydrous sodium sulfate, filtered, and concentrated to dryness. The residue was prepared by high performance liquid chromatography (HPLC) with the following parameters: column: XBridge-1 5 μm 19-150 mm; mobile phase: acetonitrile-water (0.1% NH3H2O); gradient: 10-55/8 min to give compound TPD005774 (1.05 mg, white solid, purity 93.818%), yield: 0.78%. LCMS (ESI) m/z calcd. for C₄₉H₅₂F₂N₁₀O₇[M+H]⁺ 931.4; found 931.4; ¹H NMR (400 MHz, CD₃ OD): δ=8.41 (d, J=6.9 Hz, 1H), 7.80 (d, J=8.8 Hz, 2H), 7.67 (s, 1H), 7.58-7.54 (m, 2H). 7.45-7.38 (m, 1H), 7.31 (d, J=8.9 Hz, 2H), 7.07 (t, J=8.8 Hz, 2H), 6.85 (s, 1H), 6.68 (d, J=6.8 Hz, 1H), 6.46 (d, J=8.4 Hz, 1H), 4.29 (d, J=6.1 Hz, 2H), 4.15 (s 3H), 3.75-3.55 (m, 4H), 3.18-3.13 (m, 4H), 2.85-2.69 (m, 4H), 2.26-2.17 (m, 4H), 2.07-2.02 (m, 3H), 1.65 (s, 3H), 1.29 (s, 9H). The compound structure is confirmed.

### Example 43: Synthesis of Compound TPD005901

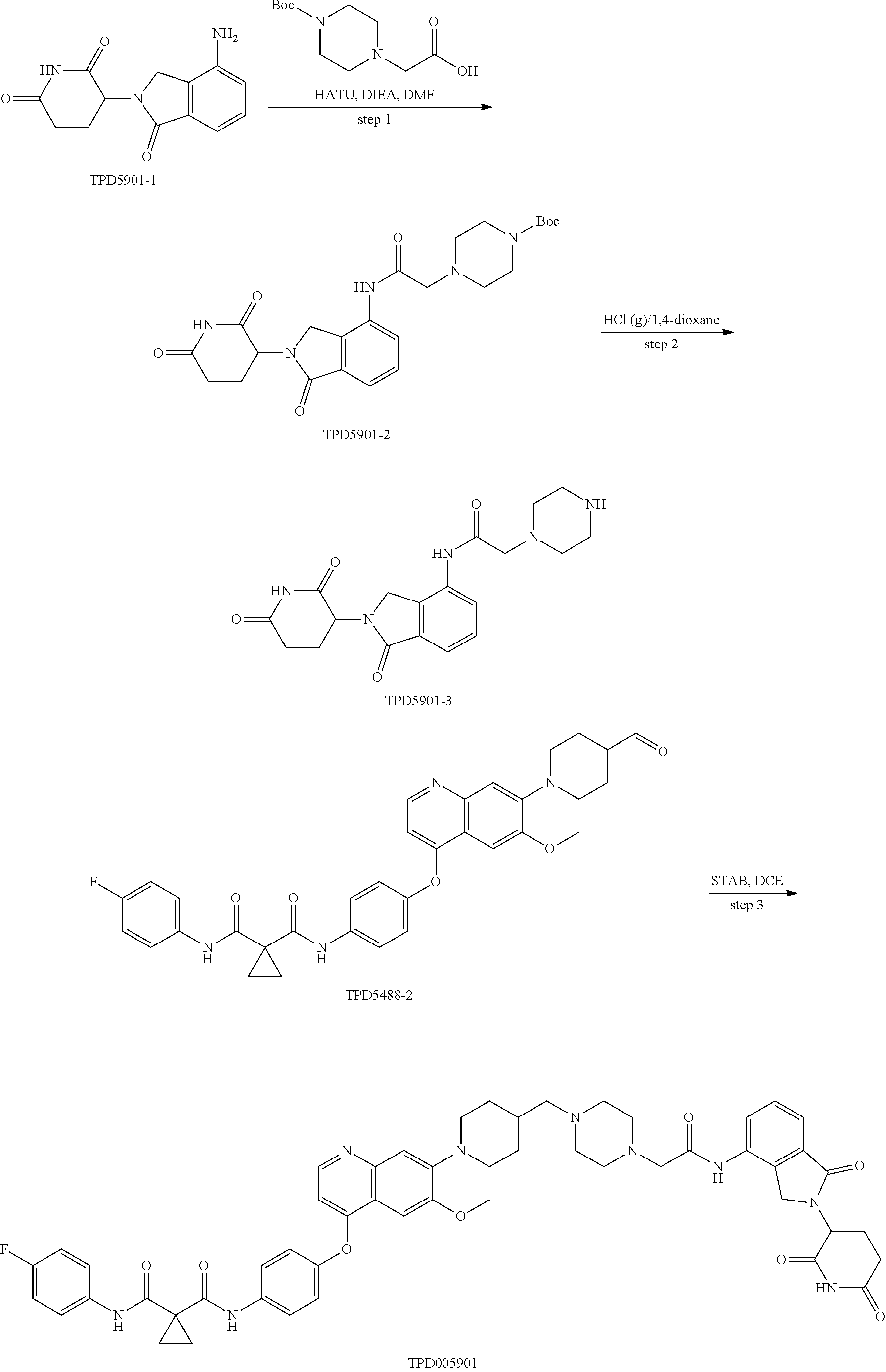 [SMILES: Nc1cccc2c1CN(C1CCC(=O)NC1=O)C2=O ; CC(C)(C)OC(=O)N1CCN(CC(=O)O)CC1 ; CC(C)(C)OC(=O)N1CCN(CC(=O)Nc2cccc3c2CN(C2CCC(=O)NC2=O)C3=O)CC1 ; O=C1CCC(N2Cc3c(NC(=O)CN4CCNCC4)cccc3C2=O)C(=O)N1 ; COc1cc2c(Oc3ccc(NC(=O)C4(C(=O)Nc5ccc(F)cc5)CC4)cc3)ccnc2cc1N1CCC(C=O)CC1 ; COc1cc2c(Oc3ccc(NC(=O)C4(C(=O)Nc5ccc(F)cc5)CC4)cc3)ccnc2cc1N1CCC(CN2CCN(CC(=O)Nc3cccc4c3CN(C3CCC(=O)NC3=O)C4=O)CC2)CC1]

To a 50 ml single-necked vial were sequentially added compound DMF (15 ml), compound TPD55901-1 (1 g, 3.90 mmol), 4-Boc-1-piperazineacetic acid (1.05 g, 4.29 mmol), HATU (2.22 g, 5.85 mmol) and DIEA (1.26 g, 9.75 mmol). The reaction was carried out under nitrogen protection at 25° C. for 16 hours. After the reaction was completed, it was poured into saturated aqueous sodium bicarbonate solution (60 ml) and extracted three times with ethyl acetate (50 ml). The organic phases were combined, washed with saturated brine, dried over anhydrous sodium sulfate, filtered, and concentrated to dryness. The residue was purified by silica gel column with eluent ratio of DCM/MeOH=50/1˜30/1 to afford the compound TPD5901-1 (1 g, white solid, purity 98.733%), yield: 51.28%. LCMS (ESI) m/z calcd. for C₂₄H₃₁N₅O₆ [M+H]⁺ 486.2; found 486.1; ¹H NMR (400 MHz, CDCl₃): δ=9.10 (s, 1H), 8.47 (s, 1H), 7.73 (dd, J=7.7, 4.5 Hz, 2H), 7.50 (t, J=7.8 Hz, 1H), 5.20 (dd, J=13.3, 5.1 Hz, 1H), 4.42 (s, 2H), 3.54-3.47 (m, 4H), 3.21 (s, 2H), 2.93-2.75 (m, 2H), 2.61 (t, J=4.8 Hz, 4H), 2.38 (dd, J=13.1, 5.3 Hz, 1H), 2.28-2.14 (m, 1H), 1.47 (s, 9H). The compound structure is confirmed.

Compound TPD5901-2 (200 mg, 0.41 mmol) and 1,4-dioxane/HCl (4N, 10 ml) were added sequentially in a 20 ml single-necked vial. The reaction was carried out at 25° C. for 4 h. After completion of the reaction, it was concentrated to dryness to give the crude compound TPD5901-3 (180 mg, brown solid), yield: 94.55 00 LCMS (ESI) m/z calcd. for C₁₉H₂₃N₅O₄ [M+H]⁺ 386.2; found 386.1. The compound structure is confirmed.

Compound TPD5488-2 (100 mg, 0.17 mmol), compound TPD5901-3 (66 mg, 0.17 mmol), DCE (4 ml) and STAB (73 mg, 0.34 mmol) were added sequentially in a 25 ml single-necked vial. The reaction was carried out under nitrogen protection at 25° C. for 16 hours. After completion of the reaction, the reaction solution was poured into saturated aqueous sodium bicarbonate solution (10 ml) and extracted three times with DCM (10 ml). The organic phases were combined, washed with saturated brine, dried over anhydrous sodium sulfate, filtered, and concentrated to dryness. The residue was purified by preparative plate with unfolding agent ratio DCM/MeOH=10/1 to give compound TPD005901 (16.7 mg, light yellow solid, purity 95.093%), yield: 19.46%. LCMS (ESI) m/z calcd. for C₅₂H₅₄FN₉O₈ [M+H]⁺ 952.4; found 952.7; ¹H NMR (400 MHz, CDCl₃): δ=9.52 (s, 1H), 9.33 (s, 1H), 8.84 (s, 1H), 8.54 (d, J=2.8 Hz, 1H), 8.27 (s, 1H), 7.82 (d, J=7.3 Hz, 2H), 7.72 (d, J=7.0 Hz, 2H), 7.67-7.52 (m, 5H), 7.25 (d, J=6.7 Hz, 2H), 7.13 (t, J=7.4 Hz, 2H), 6.53 (d, J=2.6 Hz, 1H), 5.30 (d, J=13.3 Hz, 1H), 4.54 (s, 2H), 4.11 (s, 3H), 3.81 (d, J=9.6 Hz, 2H), 3.28 (s, 2H), 3.09-2.88 (m, 2H), 2.79 (br. s., 6H), 2.64 (br. s., 3H), 2.56-2.28 (m, 5H), 2.00 (d, J=12.5 Hz, 3H), 1.89-1.73 (m, 9H), 1.67-1.55 (m, 3H). The compound structure is confirmed.

### Example 44: Synthesis of Compound TPD005907

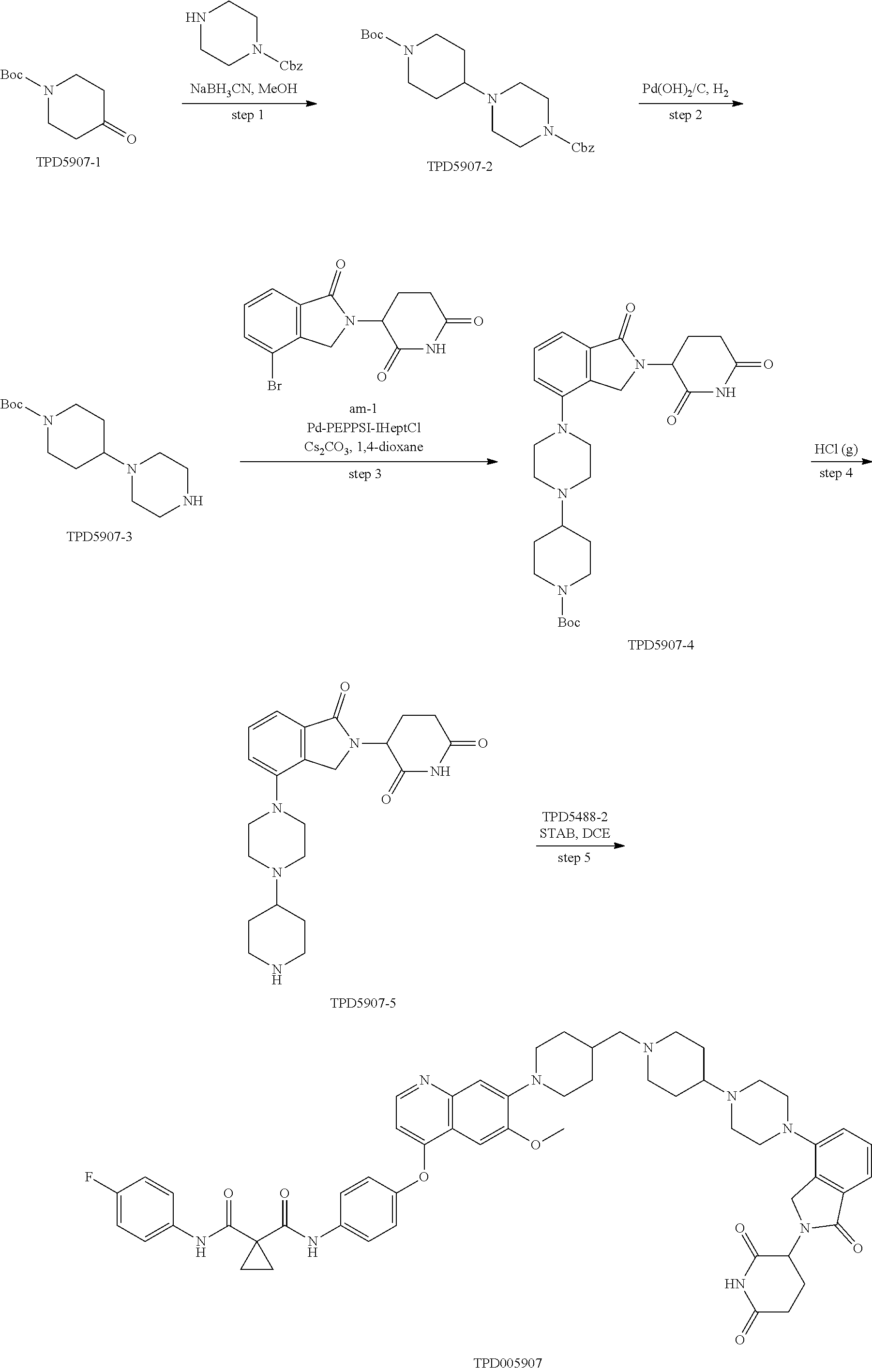

To a 100 ml single-necked vial was sequentially added compound TPD5907-1 (2.0 g, 0.01 mol), benzyl-1-piperazine carbonate (2.20 g, 0.01 mol), sodium cyanoborohydride (1.26 g, 0.02 mol) and methanol (30 ml). The reaction was carried out under nitrogen protection at 25° C. for 16 hours. After the reaction was completed, the reaction solution was poured into water (30 ml) and an appropriate amount of saturated NaHCO₃ solution was added to make the reaction solution alkaline. It was extracted three times with DCM (30 ml). The organic phases were combined, washed with saturated brine, dried over anhydrous sodium sulfate, filtered, and concentrated to dryness. The residue was purified by silica gel column with eluent ratio of DCM/MeOH=100/1˜30/1 to afford the compound TPD5907-2 (3.5 g, colorless oil, purity 56.272%), yield: 49.00%. LCMS (ESI) m/z calcd. for C₂₂H₃₃N₃O₄ [M+H]⁺ 404.2; found 404.1. The compound structure is confirmed.

To a 100 ml single-necked vial were sequentially added compounds TPD5907-2 (1.5 g, 0.0037 mol), Pd(OH)₂ (3 g, 10%) and EA (25 ml). The reaction was carried out under hydrogen atmosphere at 50° C. for 16 hours. After the reaction was completed, the reaction solution was brought to room temperature, filtered and concentrated to dryness. The residue was purified by silica gel column with eluent ratio of DCM/MeOH=20/1˜4/1 to afford the compound TPD5907-3 (0.75 g, yellow solid), yield: 67.57%. LCMS (ESI) m/z calcd. for C₁₄H₂₇N₃O₂ [M+H]⁺ 270.21; found 270.1; 1H NMR (400 MHz, DMSO_d₆): δ=3.93 (d, J=11.1 Hz, 1H), 2.80-2.58 (m, 3H), 2.47-2.37 (m, 2H), 2.35-2.25 (m, OH), 1.69 (d, J=12.7 Hz, 1H), 1.38 (s, 4H), 1.29-1.17 (m, 1H).

Compound TPD5907-3 (482 mg, 1.78 mmol), sm-1 (480 mg, 1.49 mmol), PEPPSI IHept-C1 (145 mg, 0.149 mmol) cesium carbonate (968 mg, 2.97 mmol), and 1,4-dioxane (25 ml) were added sequentially to a 25 ml single-necked vial. The reaction was carried out under nitrogen protection at 100° C. for 16 hours. After completion of the reaction, the reaction solution was brought to room temperature, decanted into water (50 ml) and extracted three times with EA (50 ml). The organic phases were combined, washed with saturated brine, dried over anhydrous sodium sulfate, filtered, and concentrated to dryness. The residue was purified by preparative silica gel column with eluent ratio of DCM/MeOH=50/1˜20/1 to afford compound TPD5907-4 (170 mg, yellow solid, purity 63.146%), yield: 14.68%. LCMS (ESI) m/z calcd. for C₂₇H₃₇N₅O₅ [M+H]⁺ 512.28; found 512.1. The compound structure is confirmed.

Compound TPD5907-4 (170 mg, 0.27 mmol) 4N HCl/1,4-dioxane (8 ml) was added sequentially in a 25 ml single-necked vial. The reaction was carried out at 25° C. for 2 h. After completion of the reaction, it was concentrated to dryness to give the crude compound TPD5907-5 (180 mg, yellow solid). LCMS (ESI) m/z calcd. for C₂₂H₂₉N₅O₃ [M+H]⁺ 412.22; found 412.1. The compound structure is confirmed.

To a 25 ml single-necked vial was sequentially added compound TPD5907-5 (177 mg, 0.43 mol), compound TPD5488-2 (250 mg, 0.43 mmol), sodium triacetoxyborohydride (1832 mg, 0.86 mmol) and DCE (8 ml). The reaction was carried out under nitrogen protection at 25° C. for 16 hours. After the reaction was completed, the reaction solution was poured into water (20 ml) and an appropriate amount of saturated NaHCO₃ solution was added to make the reaction solution alkaline. It was extracted twice with DCM (20 ml). The organic phases were combined, washed with saturated brine, dried over anhydrous sodium sulfate, filtered, and concentrated to dryness. The residue was prepared by high performance liquid chromatography (HPLC) with the following parameters: column: XBridge-1 5 μm 19-150 mm; mobile phase: acetonitrile-water (0.1% FA); gradient: 10-55/8 min to give compound TPD005907 (10.8 mg, yellow solid, purity 96.778%) in yield: 2.49%. LCMS (ESI) m/z calcd. for C₅₅H₆₀FN₉O₇ [M+H]⁺ 978.46; found 489.7, 978.4; ¹H NMR (400 MHz, CD₃ OD): δ=8.41 (d, J=5.4 Hz, 2H), 7.72 (d, J=8.8 Hz, 2H), 7.63 (s, 1H), 7.56 (dd, J=8.9 4.9 Hz, 2H), 7.48 (t, J=6.9 Hz, 2H), 7.41 (s, 1H), 7.24 (dd, J=19.1, 7.4 Hz, 3H), 7.07 (t, J=8.7 Hz, 2H), 6.53 (d, J=5.4 Hz, 1H), 5.17 (dd, J=13.2, 5.1 Hz, 1H). 4.87 (s, 1H), 4.50 (d, J=17.1 Hz, 2H), 4.03 (s, 3H), 3.74 (d, J=9.6 Hz, 2H), 3.54 (s, 2H), 3.20 (s, 4H), 3.01-2.63 (m, 13H), 2.59-2.45 (m, 1H), 2.25-2.12 (m, 3H), 2.09-2.01 (m, 1H), 2.01-1.83 (m, 4H), 1.70-1.52 (m, 6H). The compound structure is confirmed.

### Example 45: Synthesis of Compound TPD005937

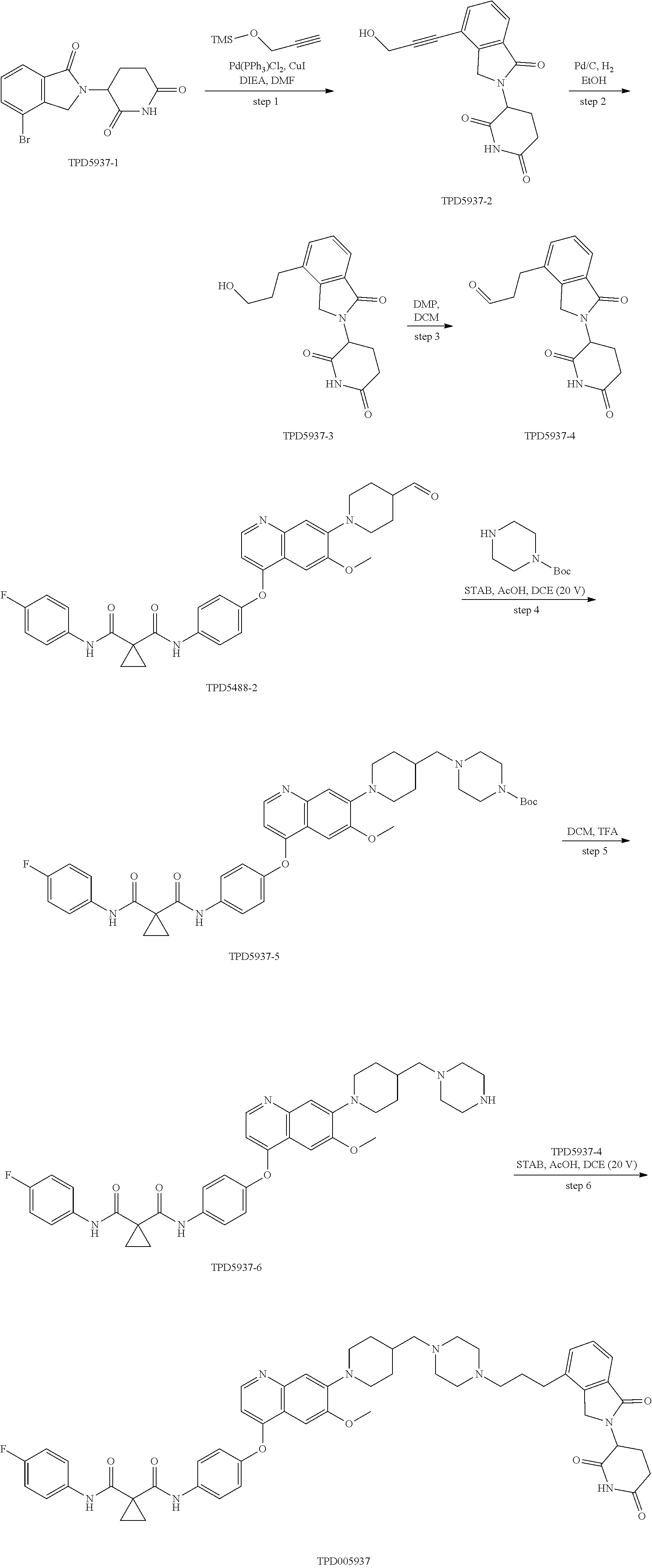

To a 50 ml triple-necked vial were sequentially added compounds TPD5937-1 (400 mg, 1.24 mmol), PdCl₂ (PPh)₃₂ (87 mg, 0.12 mmol), CuI (24 mg, 0.12 mmol), DMF (16 ml), propargyl oxytrimethylsilane (238 mg, 1.86 mmol) and DIEA (320 mg, 2.48 mmol). The reaction was carried out under nitrogen protection at 80° C. for 6 hours. After the reaction was completed the reaction solution was poured into water (10 ml) and extracted six times with ethyl acetate (5 ml). The organic phases were combined, washed with saturated brine, dried over anhydrous sodium sulfate, filtered, and concentrated to dryness, and the residue was purified by silica gel chromatographic column with eluent ratio of DCM/MeOH=100/1˜30/1 to afford the compound TPD5937-2 (250 mg, yellow solid, purity 95.746%), yield: 69.59%. LCMS (ESI) m/z calcd. for C₁₆H₁₄N₂O₄ [M+H]⁺ 299.1; found 299.0; ¹H NMR (400 MHz, DMSO_d₆): δ=11.01 (s, 1H), 7.74 (d, J=6.9 Hz, 1H), 7.69-7.66 (m, 1H), 7.54 (t, J=7.6 Hz, 1H), 5.43 (t. J=5.9 Hz, 1H), 5.17-5.13 (m, 1H), 4.35-4.33 (m, 2H), 3.16 (d, J=5.2 Hz, 2H), 3.13-3.10 (m, 1H), 2.95-2.86 (m, 1H), 2.58 (d, J=17.3 Hz, 1H), 2.46-2.38 (m, 1H), 2.03-1.97 (m, 1H). The compound structure is confirmed.

A 300 ml hydrogenation flask was charged with compound TPD5937-2 (800 mg, 2.68 mmol), Pd/C (800 mg, ethanol washed to remove water) and ethanol (80 ml). The reaction was carried out for 72 hours after passing 20-40 psi hydrogen gas and heating up to 50° C. The reaction was completed by filtration. The reaction was completed by filtration and the filter cake was washed with a large amount of ethanol. The filtrate was concentrated to dryness to afford the compound TPD5937-3 (220 mg, white solid, purity 54.175%), yield: 14.70%; LCMS (ESI) m/z calcd. for C₁₆H₁₈N₂O₄ [M+H]⁺ 303.1; found 303.0; ¹H NMR (400 MHz, DMSO_d₆): δ=11.01 (s, 1H), 7.58-7.55 (m, 1H), 7.48-7.44 (m, 2H), 5.17-5.12 (m, 1H), 4.49-4.29 (m, 2H), 3.46-3.40 (m, 3H), 2.76-2.53 (m, 4H), 2.46-2.30 (m, 2H), 1.83-1.68 (m, 2H). The compound structure is confirmed.

A 100 ml triple-necked vial was charged with compound TPD5937-3 (290 mg, 0.96 mmol) and DCM (25 ml). The reaction solution was cooled down to 0° C. under nitrogen protection and then DMP (814 mg, 1.92 mmol) was added and the reaction was carried out at 30° C. for 3 hours. After the reaction was completed the reaction solution was poured into saturated aqueous sodium bicarbonate and sodium thiosulfate (50 ml), the organic phase was separated and the aqueous phase was extracted three times with DCM (50 ml). The organic phases were combined, washed with saturated brine, dried over anhydrous sodium sulfate, filtered, and concentrated to dryness to obtain the crude compound TPD5937-4 (52 mg, white solid, purity 87.420%), yield: 15.78%. LCMS (ESI) m/z calcd. for C₁₆H₁₆N₂O₄ [M+H]⁺ 301.1; found 301.0; ¹H NMR (400 MHz, DMSO_d₆): δ=11.02 (s, 1H), 9.73 (s, 1H), 7.59-7.56 (m, 1H), 7.52-7.40 (m, 2H), 5.16-5.12 (m, 1H), 4.54-4.31 (m, 2H), 2.98-2.86 (m, 4H), 2.61 (d, J=17.5 Hz, 1H), 2.47-2.37 (m, 2H), 2.07-1.95 (m, 1H). The compound structure is confirmed.

To a 25 ml triple-necked vial were sequentially added compound TPD5488-2 (170 mg, 0.29 mmol), DCE (3.4 ml), tert-butyl piperazine-1-carboxylate (60 mg, 0.32 mmol), glacial acetic acid (53 mg, 0.88 mmol) and sodium triacetoxyborohydride (124 mg, 0.58 mmol). The reaction was carried out under nitrogen protection at 30° C. for 2 hours. The reaction solution was poured into saturated aqueous sodium bicarbonate solution (10 ml). The organic phase was separated and the aqueous phase was extracted three times with DCM (10 ml). The organic phases were combined, washed with saturated brine, dried over anhydrous sodium sulfate, filtered, and concentrated to dryness. The residue was purified by preparative plate with the unfolding agent ratio of DCM/MeOH=10/1 to afford the compound TPD5937-5 (120 mg, yellow solid, purity 97.751%), yield: 53.32%. LCMS (ESI) m/z calcd. for C₄₂H₄₉FN₆O₆ [M+H]⁺ 753.4; found 753.3; ¹H NMR (400 MHz, DMSO_d₆): δ=10.21-10.02 (m, 2H), 8.43 (d, J=5.2 Hz, 1H), 7.76 (d, J=8.8 Hz, 2H), 7.66-7.62 (m, 2H), 7.46 (s, 1H), 7.31 (s, 1H), 7.25-7.11 (m, 4H). 6.39 (d, J=5.2 Hz, 1H), 3.94 (s, 3H), 3.59 (d, J=11.2 Hz, 2H), 3.33 (d, J=3.2 Hz, 6H), 2.64 (t, J=11.1 Hz, 2H), 2.31 (br. s., 4H), 2.21 (d, J=6.8 Hz, 2H), 1.83 (d J=11.9 Hz, 2H), 1.71 (br. s., 1H), 1.47 (s, 4H), 1.40 (s, 9H), 1.33-1.21 (m, 2H). The compound structure is confirmed.

To a 25 ml single-necked vial were sequentially added compound TPD5937-5 (90 mg, 0.12 mmol), dichloromethane (3 ml) and trifluoroacetic acid (1.5 ml). The reaction was carried out at 30° C. for 2 h. After completion of the reaction, the pH was adjusted to 8 with saturated sodium bicarbonate, the organic phase was separated, and the aqueous phase was extracted three times with DCM (10 ml). The organic phases were combined, washed with saturated brine, dried over anhydrous sodium sulfate, filtered, and concentrated to the dry compound TPD5937-6 (82 mg, yellow solid, purity 93.306%), yield: 98.16%. LCMS (ESI) m/z calcd. for C₃₇H₄₁FN₆O₄ [M+H]⁺ 653.3; found 653.4; ¹H NMR (400 MHz, DMSO_d MHz, DMSO_d₆): δ=10.13 (d, J=48.7 Hz, 2H), 8.43 (d, J=5.2 Hz, 1H), 7.76 (d, J=8.9 Hz, 2H), 7.66-7.63 (m, 2H), 7.46 (s, 1H), 7.31 (s, 1H), 7.24-7.12 (m, 4H). 6.39 (d, J=5.2 Hz, 1H), 3.94 (s, 3H), 3.58 (d, J=11.5 Hz, 2H), 2.74 (s, 4H), 2.63 (t, J=11.1 Hz, 2H), 2.32 (br. s., 4H), 2.17 (d, J=7.1 Hz, 2H), 1.82 (d, J=12.1 Hz, 2H), 1.70 (br. s., 1H), 1.48 (s, 4H), 1.35-1.24 (m, 3H). The compound structure is confirmed.

To a 25 ml triple-necked vial were sequentially added compound TPD5488-2 (82 mg, 0.13 mmol), DCE (6 ml), compound TPD5937-4 (41 mg, 0.14 mmol), glacial acetic acid (23 mg, 0.38 mmol) and sodium triacetoxyborohydride (53 mg, 0.25 mmol). The reaction was carried out under nitrogen protection at 25° C. for 16 hours. The reaction solution was poured into saturated aqueous sodium bicarbonate solution (10 ml). The organic phase was separated and the aqueous phase was extracted three times with DCM (10 ml). The organic phases were combined, washed with saturated brine, dried over anhydrous sodium sulfate, filtered, and concentrated to dryness. The residue was purified by preparative plate with unfolding agent ratio DCM/MeOH=10/1 to give compound TPD005937 (39 mg, yellow solid, purity 99.422%), yield: 32.96%. LCMS (ESI) m/z calcd. for C₅₃H₅₇FN₈O₇ [M+H]⁺ 937.4; found 937.4; ¹H NMR (400 MHz, DMSO_d₆): δ=11.02 (s, 1H), 10.13 (d, J=49.4 Hz, 2H), 8.43 (d, J=5.2 Hz, 1H), 7.76 (d, J=8.9 Hz, 2H), 7.66-7.63 (m, 2H), 7.58-7.56 (m, 1H), 7.50-7.42 (m, 3H), 7.31 (s, 1H), 7.25-7.10 (m, 4H), 6.39 (d, J=5.2 Hz, 1H), 5.17-5.13 (m, 1H), 4.51-4.31 (m, 2H), 3.94 (s, 3H), 3.58 (d, J=11.1 Hz, 2H), 2.98-2.88 (m, 1H), 2.68-2.61 (m, 5H), 2.45-2.25 (m, 8H), 2.18 (d, J=6.3 Hz, 2H), 2.05-2.00 (m, 1H), 1.82-1.75 (m, 4H), 1.47 (s, 4H), 1.30-1.19 (m, 6H). The compound structure is confirmed.

### Example 46: Synthesis of Compound TPD005937-Trifluoroacetate

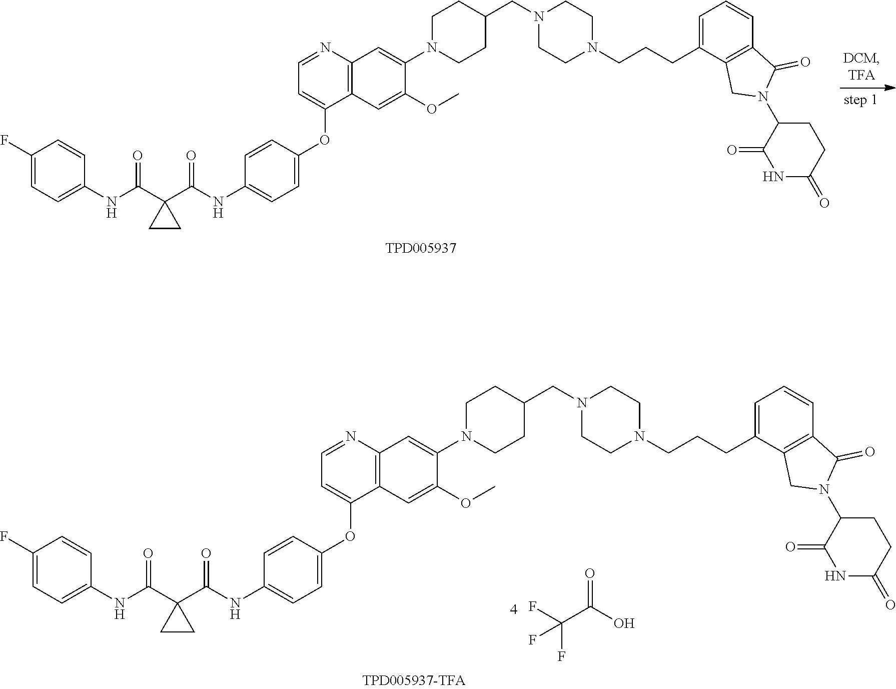

Compound TPD005937 (10 mg, 0.0097 mmol), DCM (2 ml) and TFA (1 ml) were added sequentially in a 25 ml single-necked vial. The system reacted at 30° C. for 10 minutes. Concentration to dryness gave the product compound TPD005937-TFA (7.4 mg, yellow solid, purity 96.678), yield: 52.58%. LCMS (ESI) m/z calcd. for C₅₃H₅₇FN₈O₇ [M+H]⁺ 937.4; found 937.4; ¹H NMR (400 MHz, DMSO_d₆): δ=11.04 (s, 1H), 10.33 (s, 1H), 10.02 (s, 1H), 8.73 (d, J=6.6 Hz, 1H), 7.85 (d, J=8.9 Hz, 2H), 7.67-7.61 (m, 4H), 7.52-7.47 (m, 3H), 7.35 (d, J=9.0 Hz, 2H), 7.16 (t, J=8.9 Hz, 2H), 6.74 (d, J=6.6 Hz, 1H), 5.20-5.15 (m, 1H), 4.52-4.31 (m, 2H), 4.05 (s, 3H), 3.79 (d, J=11.0 Hz, 2H), 3.06-2.90 (m, 4H), 2.84 (t, J=11.5 Hz, 2H), 2.73-2.68 (m, 2H), 2.66-2.55 (m, 2H), 2.46-2.24 (m, 3H), 2.22-1.74 (m, 8H), 1.49 (d, J=6.5 Hz, 4H), 1.37 (d, J=11.2 Hz, 2H), 1.23 (s, 4H). The compound structure is confirmed.

### Example 47: Synthesis of Compound TPD005972

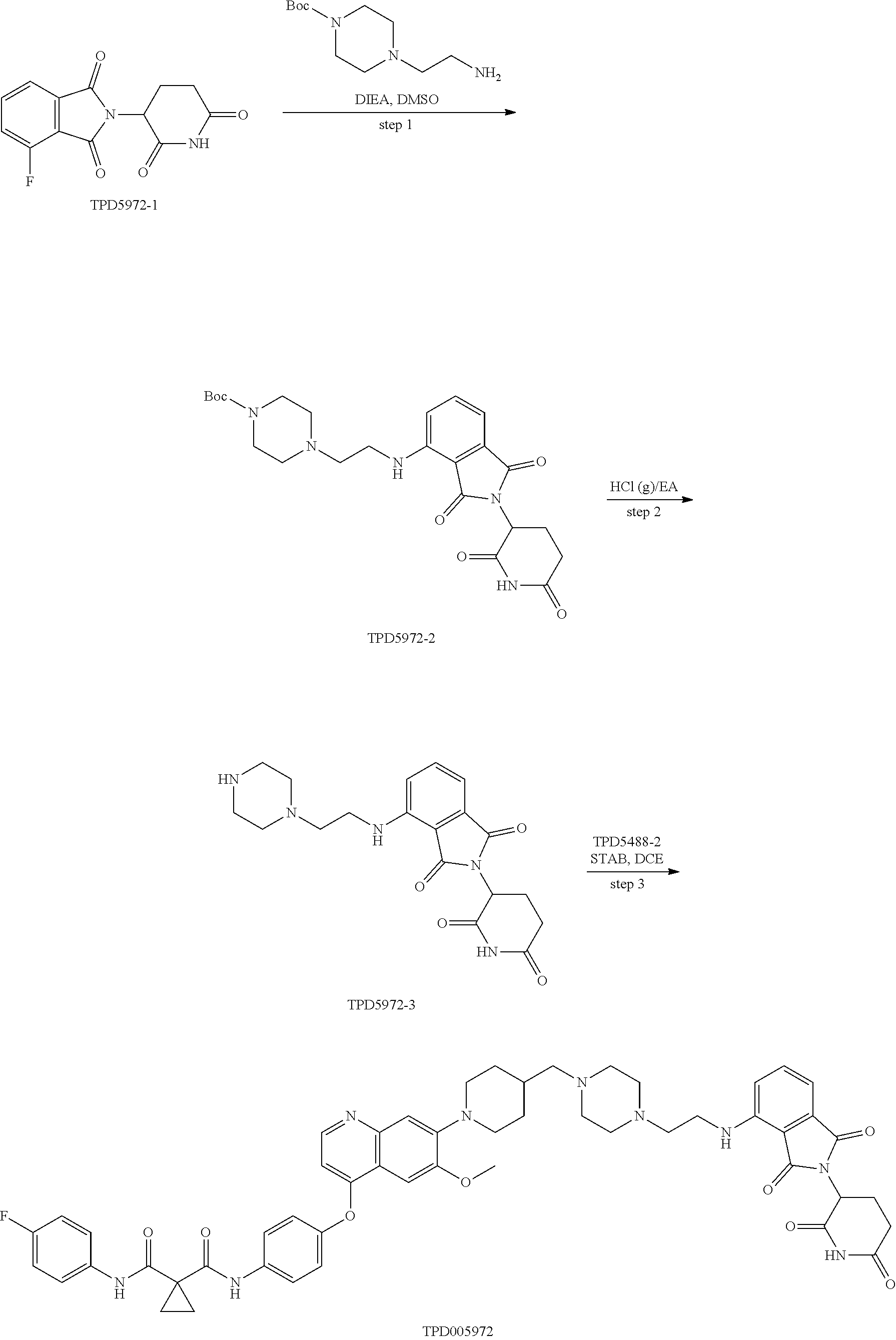 [SMILES: O=C1CCC(N2C(=O)c3cccc(F)c3C2=O)C(=O)N1 ; CC(C)(C)OC(=O)N1CCN(CCN)CC1 ; CC(C)(C)OC(=O)N1CCN(CCNc2cccc3c2C(=O)N(C2CCC(=O)NC2=O)C3=O)CC1 ; O=C1CCC(N2C(=O)c3cccc(NCCN4CCNCC4)c3C2=O)C(=O)N1 ; COc1cc2c(Oc3ccc(NC(=O)C4(C(=O)Nc5ccc(F)cc5)CC4)cc3)ccnc2cc1N1CCC(CN2CCN(CCNc3cccc4c3C(=O)N(C3CCC(=O)NC3=O)C4=O)CC2)CC1]

To a 50 ml single-necked vial were sequentially added compound DMSO (10 ml), compound TPD55972-1 (500 mg, 1.81 mmol), 4-N-(2-aminoethyl)-1-N-Boc-piperidine (500 mg, 2.17 mmol) and DIEA (468 mg, 3.62 mmol). The reaction was carried out under nitrogen protection at 100° C. for 16 hours. After the reaction was completed, it was poured into saturated aqueous sodium bicarbonate solution (30 ml) and extracted three times with ethyl acetate (20 ml). The organic phases were combined, washed with saturated brine, dried over anhydrous sodium sulfate, filtered, and concentrated to dryness. The residue was purified by silica gel column with eluent ratio of DCM/MeOH=100/1˜60/1 to afford the compound TPD5972-2 (420 mg, yellow solid, purity 97.762%), yield: 46.62%. LCMS (ESI) m/z calcd. for C₂₄H₃₁N₅O₆[M+H]⁺ 486.2; found 486.1; ¹H NMR (400 MHz, CDCl₃): δ=11.10 (s, 1H), 7.63-7.56 (m, 1H), 7.10 (d, J=8.6 Hz, 1H), 7.04 (d, J=7.0 Hz, 1H), 6.79 (t, J=4.7 Hz, 1H), 5.07 (dd, J=12.9, 5.3 Hz, 1H), 3.42-3.28 (m, 8H), 2.95-2.80 (m, 1H), 2.65-2.56 (m, 3H), 2.43-2.34 (m, 4H), 2.09-1.96 (m, 1H), 1.37 (s, 9H). The compound structure is confirmed.

To a 20 ml single-necked vial was sequentially added compound TPD5972-2 (420 mg, 0.86 mmol) and HCl(g)/EA (4N, 10 ml). The reaction was carried out at 25° C. for 2 h. After completion of the reaction, it was concentrated to dryness to give the crude compound TPD5972-3 (360 mg, brown solid), yield: 97.87%. LCMS (ESI) m/z calcd. for C₁₉H₂₃N₅O₄ [M+H]⁺ 386.2; found 386.1. The compound structure is confirmed.

Compound TPD5488-2 (100 mg, 0.17 mmol), compound TPD5972-3 (79 mg, 0.21 mmol), DCE (4 ml) and STAB (73 mg, 0.34 mmol) were added sequentially in a 25 ml single-necked vial. The reaction was carried out under nitrogen protection at 25° C. for 16 hours. After the reaction was completed, the reaction solution was poured into saturated aqueous sodium bicarbonate solution (10 ml) and extracted three times with DCM (10 ml). The organic phases were combined, washed with saturated brine, dried over anhydrous sodium sulfate, filtered, and concentrated to dryness. The residue was purified by preparative plate with unfolding agent ratio DCM/MeOH=10/1 to afford compound TPD005972 (12.4 mg, yellow solid, purity 95.422%), yield: 7.23%. LCMS (ESI) m/z calcd. for C₅₂H₅₄FN₉O₈ [M+H]⁺ 952.4; found 952.7; ¹H NMR (400 MHz, CDCl₃): δ=11.11 (s, 1H), 10.18 (s, 1H), 10.06 (s, 1H), 8.43 (d, J=5.2 Hz, 1H), 7.76 (d, J=8.9 Hz, 2H), 7.68-7.54 (m, 3H), 7.46 (s, 1H), 7.31 (s, 1H). 7.26-7.07 (m, 5H), 7.03 (d, J=7.1 Hz, 1H), 6.77 (t, J=4.6 Hz, 1H), 6.39 (d, J=5.2 Hz, 1H), 5.08 (dd, J=13.0, 5.4 Hz, 1H), 4.22 (t, J=6.6 Hz, 1H), 3.94 (s, 4H). 3.59 (d, J=11.3 Hz, 2H), 3.43-3.35 (m, 3H), 2.95-2.81 (m, 1H), 2.71-2.55 (m, 6H), 2.43-2.35 (m, 4H), 2.21 (d, J=6.9 Hz, 2H), 2.06-1.97 (m, 1H), 1.83 (d, J=11.4 Hz, 2H), 1.76-1.51 (m, 3H), 1.47 (s, 4H), 1.41-1.25 (m, 4H), 0.95-0.84 (m, 2H). The compound structure is confirmed.

### Example 48: Synthesis of Compound TPD007022

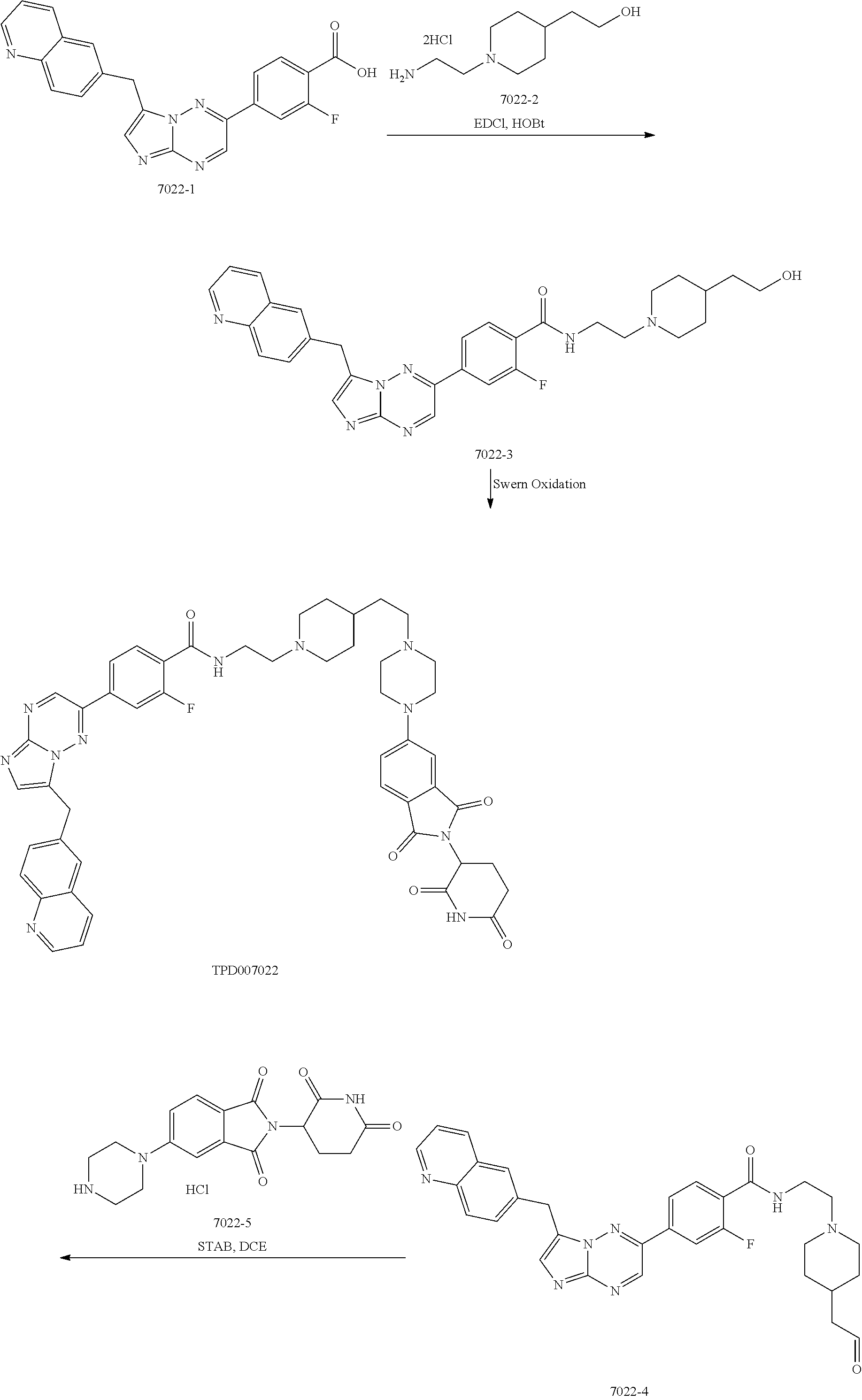

Compound 7022-1 (500 mg) underwent amide condensation with intermediate 7022-2 to give compound 7022-3. This product was oxidized to prepare its corresponding aldehyde derivative 7022-4 (100 mg). compounds TPD7022-5 (81 mg), DCE (4 ml) and STAB (80 mg) were added sequentially to a 25 ml single mouth vial. The reaction was carried out under nitrogen protection at 25° C. for 16 hours. After the reaction was completed, the reaction solution was poured into saturated aqueous sodium bicarbonate solution (10 ml) and extracted three times with DCM (10 ml). The organic phases were combined, washed with saturated brine, dried over anhydrous sodium sulfate, filtered, and concentrated to dryness. The residue was purified by preparative plate with unfolding agent ratio DCM/MeOH=10/1 to give compound TPD007022 (17 mg, yellow solid, purity 97%). LCMS (ESI) m/z calcd. for C₄₈H₄₉FN₁₁O₅ [M+H]⁺ 877.4; found 877.7. The compound structure is confirmed.

### Example 49: Synthesis of compound TPD007026

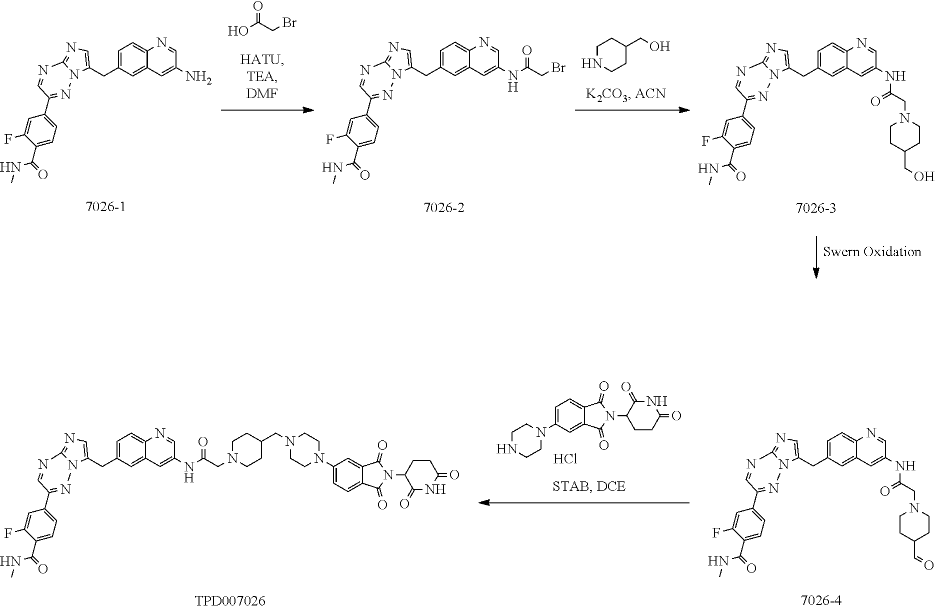

Compound 7026-1 (700 mg) underwent a condensation reaction with bromoacetic acid amide to give compound 7026-2. This product was condensed with 4-hydroxymethylpiperidine under alkaline conditions to give 7026-3, followed by oxidation to prepare its corresponding aldehyde derivative 7026-4 (90 mg). It was added to a 25 ml single-necked vial and compound TPD7022-5 (75 mg), DCE (4 ml) and STAB (77 mg) were added sequentially. The reaction was carried out under nitrogen protection at 25° C. for 16 hours. After completion of the reaction, the reaction solution was poured into saturated aqueous sodium bicarbonate solution (10 ml) and extracted three times with DCM (10 ml). The organic phases were combined, washed with saturated brine, dried over anhydrous sodium sulfate, filtered, and concentrated to dryness. The residue was purified by preparative plate with unfolding agent ratio DCM/MeOH=10/1 to give compound TPD007022 (15 mg, light yellow solid, purity 97%). LCMS (ESI) m/z calcd. for C₄₈H₄₈FN₁₂O₆ [M+H]⁺ 907.4; found 907.6. The compound structure is confirmed.

### Example 50: Synthesis of Compound TPD0090

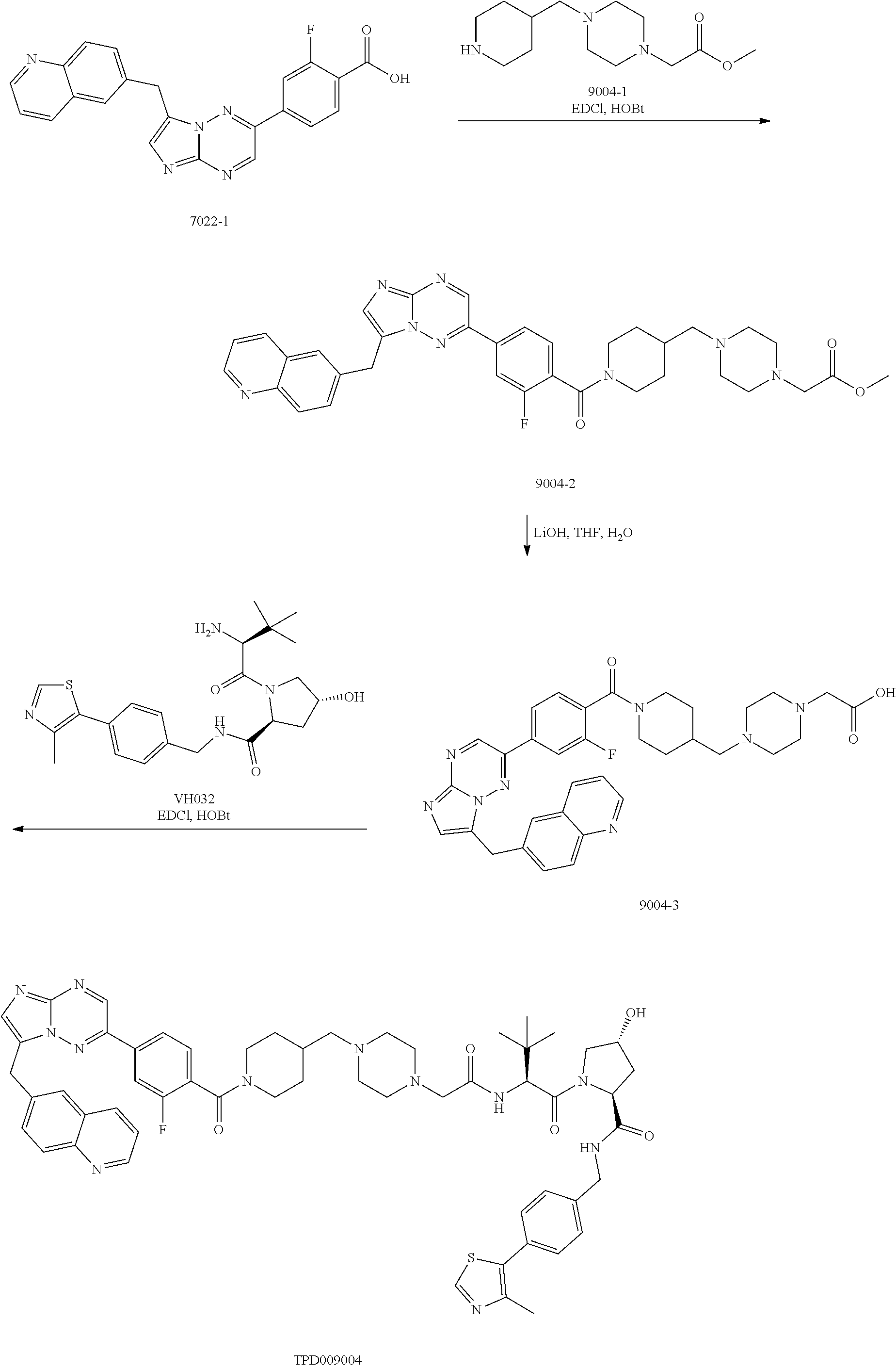 [SMILES: O=C(O)c1ccc(-c2cnc3ncc(Cc4ccc5ncccc5c4)n3n2)cc1F ; COC(=O)CN1CCN(CC2CCNCC2)CC1 ; COC(=O)CN1CCN(CC2CCN(C(=O)c3ccc(-c4cnc5ncc(Cc6ccc7ncccc7c6)n5n4)cc3F)CC2)CC1 ; Cc1ncsc1-c1ccc(CNC(=O)[C@@H]2C[C@@H](O)CN2C(=O)[C@@H](N)C(C)(C)C)cc1 ; O=C(O)CN1CCN(CC2CCN(C(=O)c3ccc(-c4cnc5ncc(Cc6ccc7ncccc7c6)n5n4)cc3F)CC2)CC1 ; Cc1ncsc1-c1ccc(CNC(=O)[C@@H]2C[C@@H](O)CN2C(=O)[C@@H](NC(=O)CN2CCN(CC3CCN(C(=O)c4ccc(-c5cnc6ncc(Cc7ccc8ncccc8c7)n6n5)cc4F)CC3)CC2)C(C)(C)C)cc1]

Compound 7022-1 (600 mg) was condensed with 9004-1 to give compound 9004-2. Hydrolysis of the product in lithium hydroxide solution gave 9004-3, which was added to a 25 ml vial and compound VH032, DCM (5 ml), HOBt (5 mg), and EDCI were added sequentially. the reaction was carried out for 15 h at 25° C. under nitrogen protection. After completion of the reaction, the reaction solution was poured into saturated aqueous sodium bicarbonate solution (10 ml) and extracted three times with DCM (10 ml). The organic phases were combined, washed with saturated brine, dried over anhydrous sodium sulfate, filtered, and concentrated to dryness. The residue was purified by preparative plate with the unfolding agent ratio of DCM/MeOH=10/1 to give the compound TPD009004 (19 mg, pale yellow solid, purity 96%). LCMS (ESI) m/z calcd. for C₅₆H₆₄FN₁₂O₅S [M+H]⁺ 1035.5; found 1035.6. The compound structure is confirmed.

### Example 51: Synthesis of Compound TPD009006

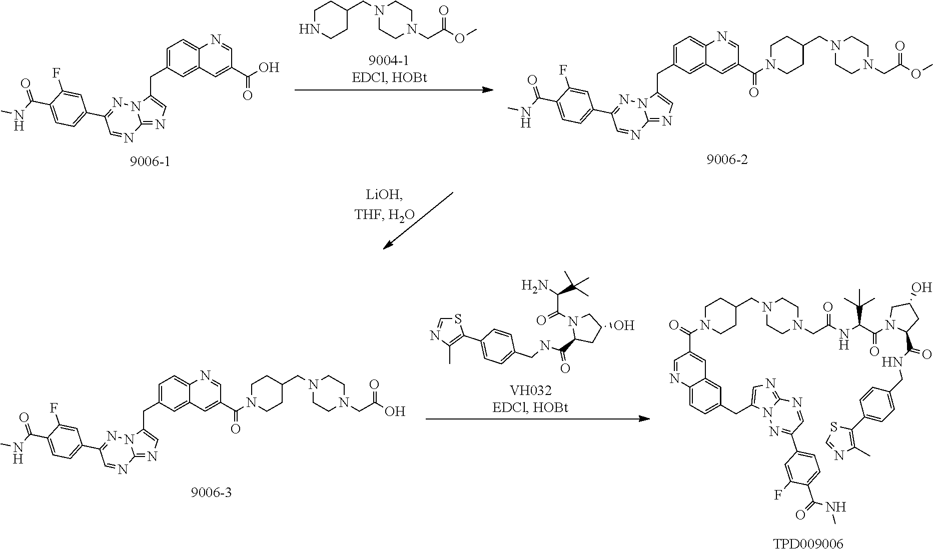

Compound 9006-1 (500 mg) reacted with EDCI, HOBt in DCM (10 ml) and amide condensation with 9004-1 to give compound 9006-2. Hydrolysis of the product in lithium hydroxide solution afforded 9006-3, which was added to a 25 ml single-necked bottle and compound VH032, DCM (5 ml), HOBt. The reaction was carried out under nitrogen protection at 25° C. for 17 hours. After completion of the reaction, the reaction solution was poured into saturated aqueous sodium bicarbonate solution (10 ml) and extracted three times with DCM (10 ml). The organic phases were combined, washed with saturated brine, dried over anhydrous sodium sulfate, filtered, and concentrated to dryness. The residue was purified by preparative plate with the unfolding agent ratio of DCM/MeOH=10/1 to give the compound TPD009006 (13 mg, pale yellow solid, purity 98%). LCMS (ESI) m/z calcd. for C₅₈H₆₇FN₁₃O₆S [M+H]⁺ 1092.5; found 1092.6. The compound structure is confirmed.

### Example 52: Synthesis of Compound TPD10001

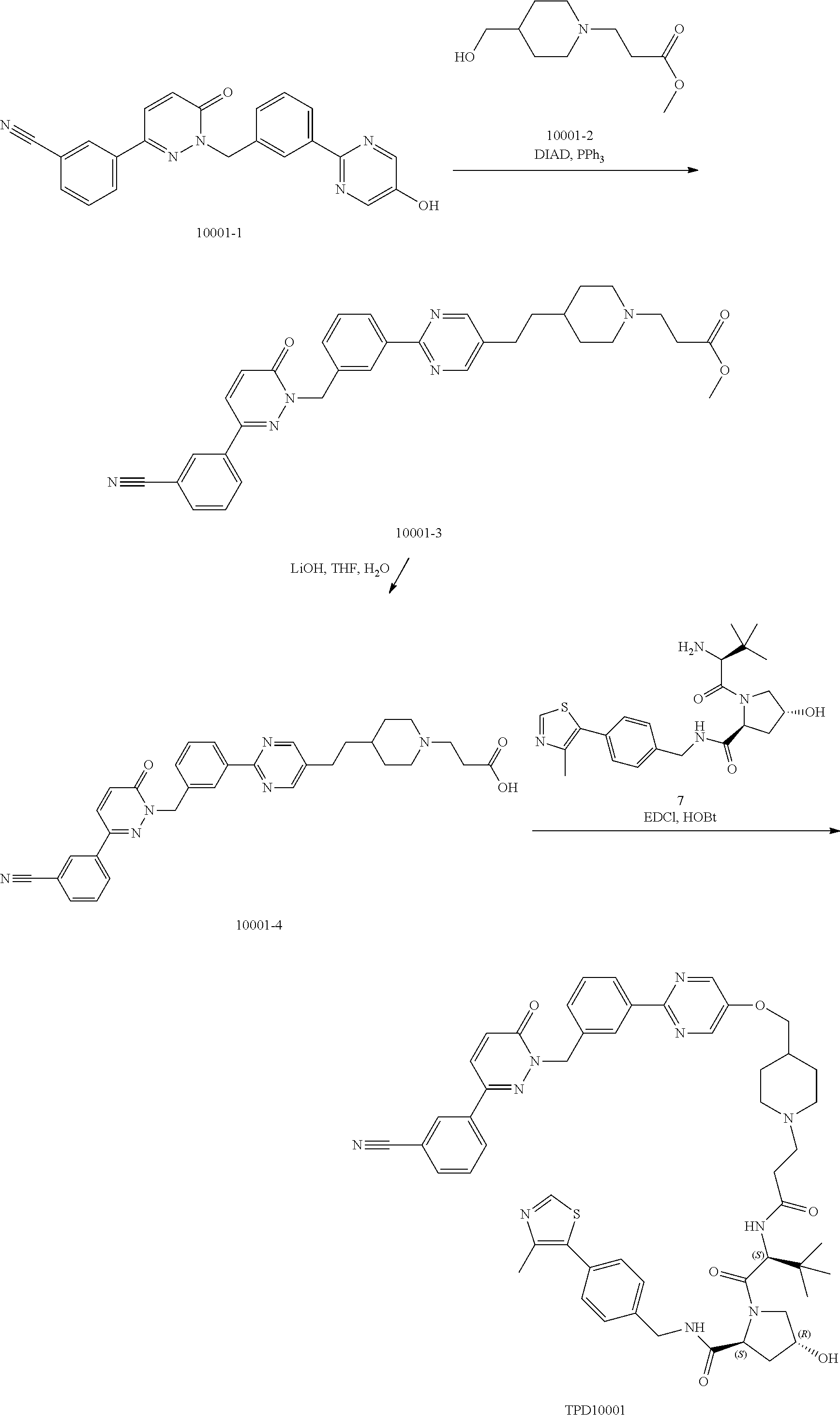

Compound 10001-1 (550 mg) reacted with DIAD, PPh3 in DCM (10 ml) and then 10001-2 was added for the condensation reaction to give compound 10001-3. the product was hydrolyzed in lithium hydroxide solution to give 10001-4, the latter was added to 25 ml of a single-necked vial and compound VH032, DCM (5 ml) was added sequentially, HOBt (5 mg) and EDCI. The reaction was carried out under nitrogen protection at 25° C. for 19 hours. After completion of the reaction, the reaction solution was poured into saturated aqueous sodium bicarbonate solution (10 ml) and extracted three times with DCM (10 ml). The organic phases were combined, washed with saturated brine, dried over anhydrous sodium sulfate, filtered, and concentrated to dryness. The residue was purified by preparative plate with unfolding agent ratio DCM/MeOH=10/1 to give compound TPD10001 (11 mg, light yellow solid, purity 98%). LCMS (ESI) m/z calcd. for C₅₃H₅₉N₁₀O₆S [M+H]⁺ 963.4; found 963.6. The compound structure is confirmed.

### Example 53: Synthesis of compound TPD10013

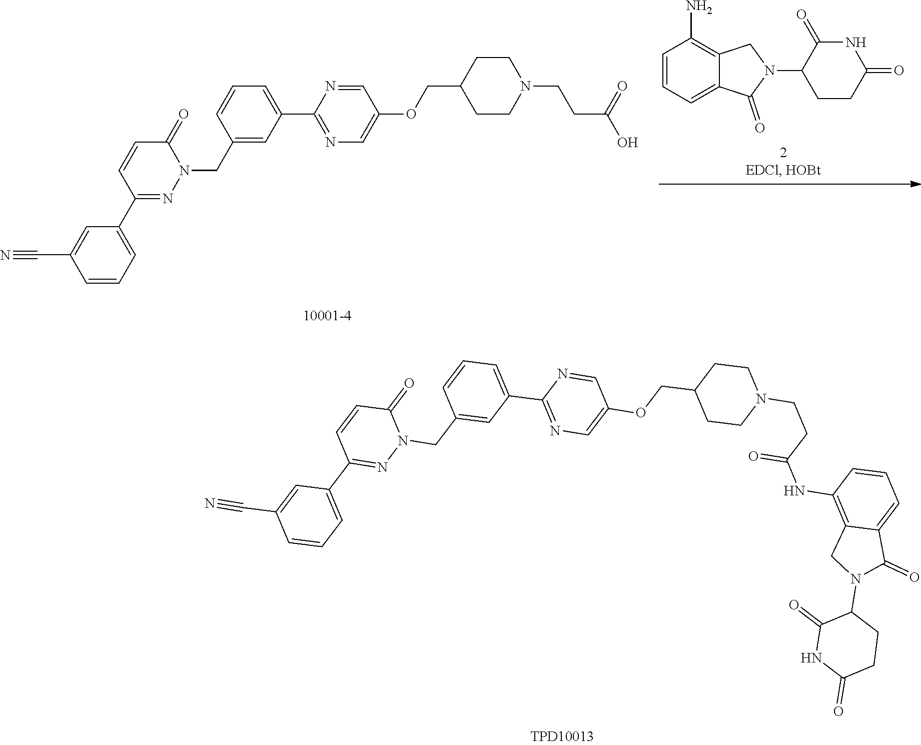

Compound 10001-4 (90 mg) reacted with compound lenalidomide, HOBt (5 mg) and EDCI in DCM (5 ml). The reaction was stirred under nitrogen protection at 25° C. for 19 hours. After completion of the reaction, the reaction solution was poured into saturated aqueous sodium bicarbonate solution (10 ml) and extracted three times with DCM (10 ml). The organic phases were combined, washed with saturated brine, dried over anhydrous sodium sulfate, filtered, and concentrated to dryness. The residue was purified by preparative plate with unfolding agent ratio DCM/MeOH=10/1 to give compound TPD10001 (17 mg, light yellow solid, purity 96%). LCMS (ESI) m/z calcd. for C44H₄₂N₉O₆ [M+H]⁺ 792.3; found 792.5. The compound structure is confirmed.

### Example 54: Synthesis of Compound TPD10040

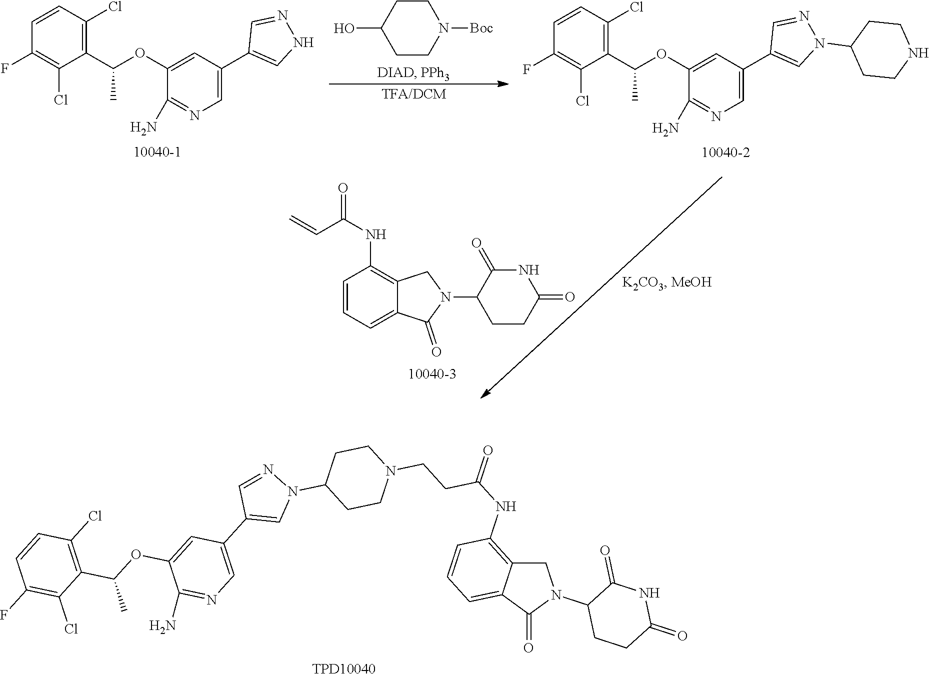

Compound 10040-4 (70 mg) reacted with DIAD, PPh3 in DCM (7 ml) followed by the addition of N-Boc-protected 4-hydroxypiperidine for the condensation reaction to give the crude product deprotected by TFA/DCM to give 10040-2. The product was added to a 25 ml single-necked vial and compound 10040-3, potassium carbonate, and methanol were added sequentially. The reaction was carried out under nitrogen protection at 25° C. for 12 hours. After completion of the reaction, the reaction solution was poured into saturated aqueous sodium bicarbonate solution (10 ml) and extracted three times with DCM (10 ml). The organic phases were combined, washed with saturated brine, dried over anhydrous sodium sulfate, filtered, and concentrated to dryness. The residue was purified by preparative plate with the unfolding agent ratio of DCM/MeOH=10/1 to give the compound TPD10040 (9 mg, pale yellow solid, 98% purity). LCMS (ESI) m/z calcd. for C₃₇H₃₈Cl₂FN₈O₅ [M+H]⁺ 763.2; found 763.3. The compound structure is confirmed.

### Example 55: Synthesis of Compound TPD10041

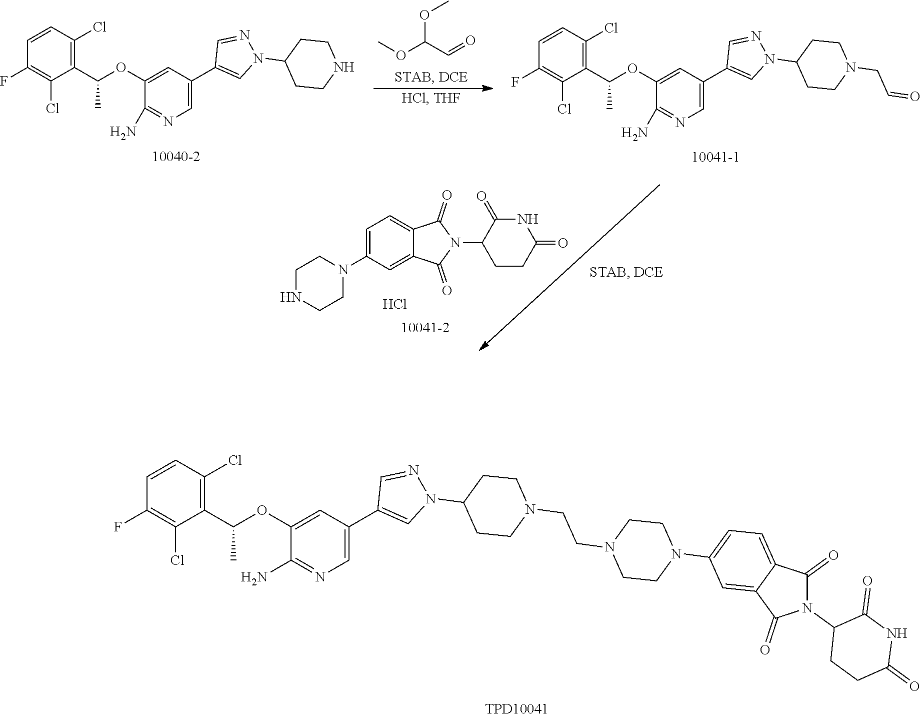

Compound 10040-2 (90 mg) reacted with STAB, 2,2-dimethoxyacetaldehyde in DCE (7 ml) and then the crude product obtained was stripped of the protecting groups in HCl/THF system to give 10041-1. The product was added to a 25 ml single mouth vial and compound 10041-2, STAB and DCE were added sequentially. nitrogen protected reaction at 25° C. for 16 h. The reaction was carried out under nitrogen protection for 16 h. The reaction was carried out under nitrogen protection at 25° C. for 16 h. The reaction was carried out under nitrogen protection at 25° C. for 16 hours. After completion of the reaction, the reaction solution was poured into saturated aqueous sodium bicarbonate solution (10 ml) and extracted three times with DCM (10 ml). The organic phases were combined, washed with saturated brine, dried over anhydrous sodium sulfate, filtered, and concentrated to dryness. The residue was purified by preparative plate with the unfolding agent ratio of DCM/MeOH=10/1 to give the compound TPD10041 (7 mg, pale yellow solid, 98% purity). LCMS (ESI) m/z calcd. for C₄₀H₄₃Cl₂FN₉O₅ [M+H]⁺ 818.3; found 818.4. The compound structure is confirmed.

### Example 56: Synthesis of Compound TPD10050

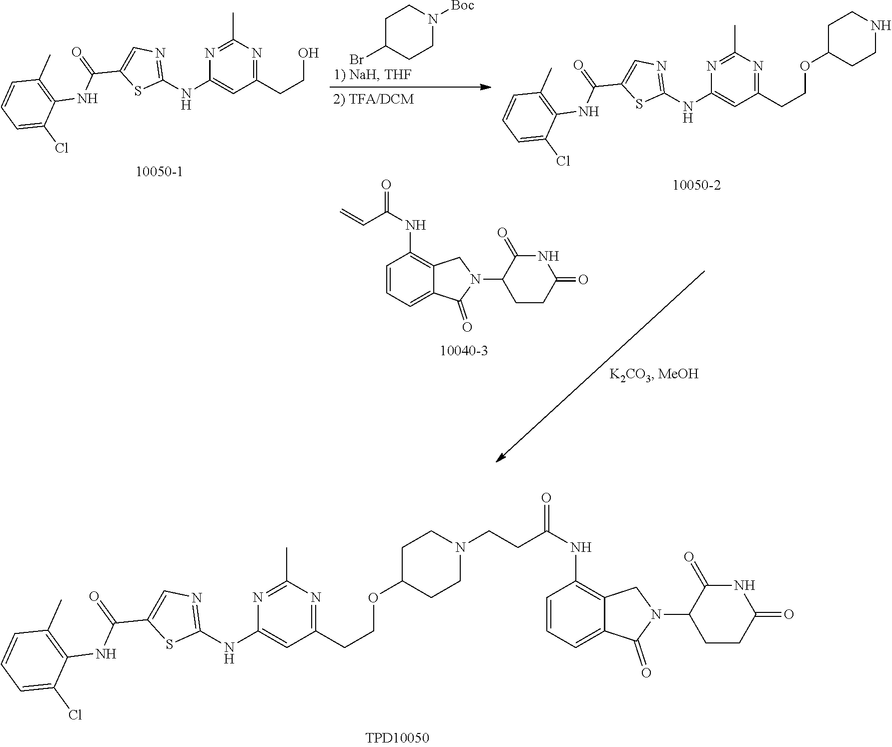 [SMILES: Cc1nc(CCO)cc(Nc2ncc(C(=O)Nc3c(C)cccc3Cl)s2)n1 ; CC(C)(C)OC(=O)N1CCC(Br)CC1 ; Cc1nc(CCOC2CCNCC2)cc(Nc2ncc(C(=O)Nc3c(C)cccc3Cl)s2)n1 ; C=CC(=O)Nc1cccc2c1CN(C1CCC(=O)NC1=O)C2=O ; Cc1nc(CCOC2CCN(CCC(=O)Nc3cccc4c3CN(C3CCC(=O)NC3=O)C4=O)CC2)cc(Nc2ncc(C(=O)Nc3c(C)cccc3Cl)s2)n1]

Compound 10050-1 (100 mg) reacted with NaH in anhydrous THF (9 ml), followed by the addition of N-Boc-4-bromopiperidine, and the crude compound obtained from the reaction reacted with TFA/DCM to remove the protecting group to give 10050-2. The product was added to a 25 mlmono flask and compound 10040-3, potassium carbonate, and methanol were added sequentially. The reaction was carried out under nitrogen protection at 25° C. for 12 hours. After completion of the reaction, the reaction solution was poured into saturated aqueous sodium bicarbonate solution (10 ml) and extracted three times with DCM (10 ml). The organic phases were combined, washed with saturated brine, dried over anhydrous sodium sulfate, filtered, and concentrated to dryness. The residue was purified by preparative plate with the unfolding agent ratio of DCM/MeOH=10/1 to give the compound TPD10040 (11 mg, light yellow solid, purity 96%). LCMS (ESI) m/z calcd. for C₃₉H₄₃C1N₉O₆S [M+H]⁺ 800.3; found 800.3. The compound structure is confirmed.

### Example 57: Synthesis of compound TPD10051

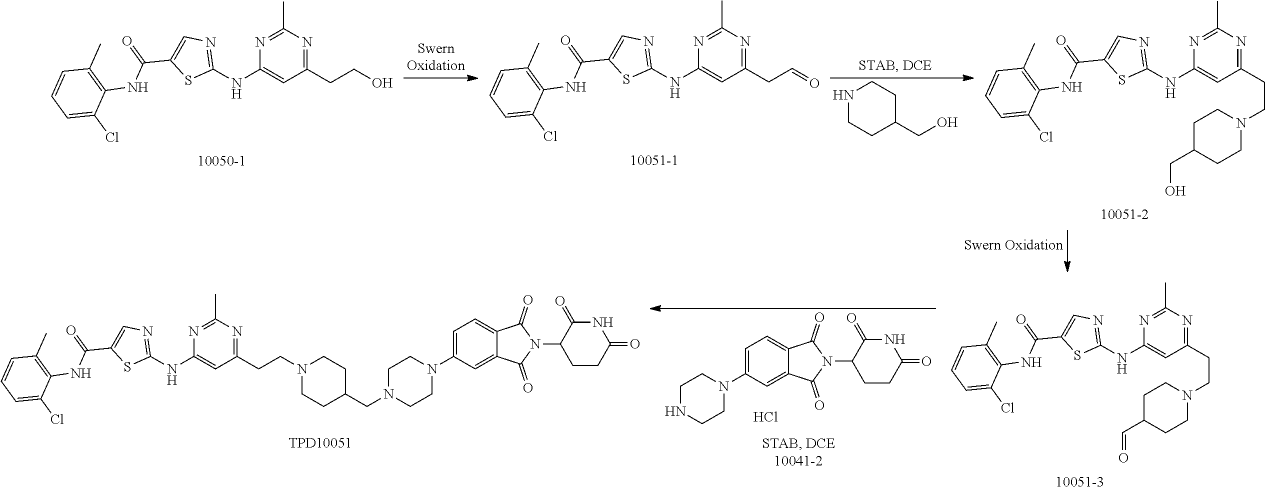

Compound 10050-1 (100 mg) was oxidized by Swern reagent to give compound 10051-1, the latter reacted with STAB, 4-hydroxymethylpiperidine in DCE (7 ml) to give 10051-2, which was then oxidized by Swern reagent to give compound 10051-3. The product was added to a 25 ml single-necked vial and compound 10041-2 was added in turn, STAB and DCE. The reaction was carried out under nitrogen protection at 25° C. for 16 hours. After completion of the reaction, the reaction solution was poured into saturated aqueous sodium bicarbonate solution (10 ml) and extracted three times with DCM (10 ml). The organic phases were combined, washed with saturated brine, dried over anhydrous sodium sulfate, filtered, and concentrated to dryness. The residue was purified by preparative plate with unfolding agent ratio DCM/MeOH=10/1 to give compound TPD 10051 (9 mg, light yellow solid, purity 98%). LCMS (ESI) m/z calcd. for C₄₁H₄₆ClN₁₀O₅S [M+H]⁺ 825.4; found 825.5. The compound structure is confirmed.

### Example 58: Synthesis of compound TPD12001

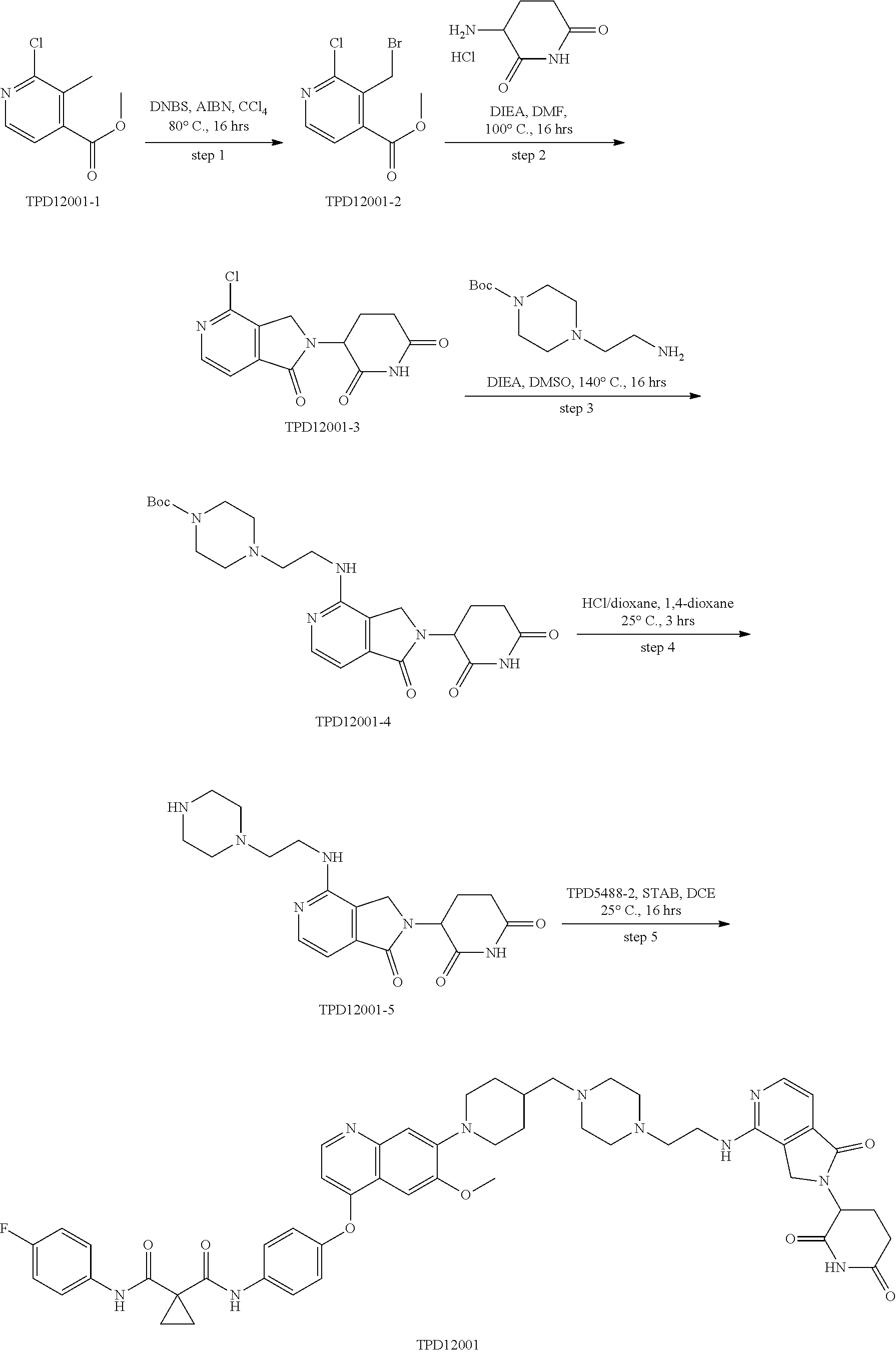

To a 100 ml triple-necked vial were sequentially added compounds TPD12001-1 (2 g, 10.8 mmol), NBS (2.88 g, 16.2 mmol), AIBN (0.18 g, 1.08 mmol), and CCl₄ (20 ml). The reaction was carried out under nitrogen protection at 80° C. for 16 hours. After completion of the reaction, the reaction solution was brought to room temperature, decanted into water (30 ml) and extracted three times with DCM (10 ml). The organic phases were combined, washed with saturated brine, dried over anhydrous sodium sulfate, filtered, and concentrated to dryness to afford the crude compound TPD12001-2 (3.1 g, yellow oil, purity 85.695%), yield: 92.59%. LCMS (ESI) m/z calcd. for C₈H₇BrClNO₂ [M+H]⁺ 263.9, 263.9; found 264.1, 266.0; ¹H NMR (400 MHz, CDCl₃): δ=8.48 (d, J=5.0 Hz, 1H), 7.71 (d, J=5.0 Hz, 1H), 5.05 (s, 2H), 4.02 (s, 3H). The compound structure is confirmed.

To a 100 ml triple-necked vial were sequentially added compound TPD12001-2 (3 g, 11.3 mmol), 3-amino-2,6-piperidinedione hydrochloride (2.23 g, 13.5 mmol), DIEA (2.92 g, 22.6 mmol) and DMF (30 ml). The reaction was carried out under nitrogen protection at 100° C. for 16 hours. After completion of the reaction, the reaction solution was reduced to room temperature, decanted into water (100 ml) and extracted three times with EA (50 ml). The organic phases were combined, washed with saturated brine, dried over anhydrous sodium sulfate, filtered, and concentrated to dryness. The residue was purified by silica gel column with eluent ratio of DCM/MeOH=100/1˜30/1 to afford the compound TPD12001-3 (1.72 g, yellow solid, purity 92.354%), yield: 50.44%. LCMS (ESI) m/z calcd. for C₁₂H₁₀ClN₃O₃ [M+H]⁺ 280.0; found 280.1; ¹H NMR (400 MHz, DMSO_d₆): δ=11.06 (s, 1H), 8.63 (d, J=4.9 Hz, 1H), 7.80 (d, J=4.9 Hz, 1H), 5.18 (dd, J=13.3, 5.1 Hz, 1H), 4.60 (d, J=18.2 Hz, 1H), 4.44 (d, J=18.3 Hz, 1H), 2.95-2.87 (m, 1H), 2.63-2.58 (m, 1H), 2.49-2.42 (m, 1H), 2.05-2.01 (m, 1H). The compound structure is confirmed.

Compound TPD12001-3 (500 mg, 1.79 mmol), 4-N-(2-aminoethyl)-1-N-BOC-piperidine (492 mg, 2.15 mmol), DIEA (462 mg, 3.58 mmol), and DMSO (10 ml) were added sequentially in 25 ml triple vials. The reaction was carried out under nitrogen protection at 140° C. for 16 hours. After completion of the reaction, the reaction solution was reduced to room temperature, decanted into water (50 ml) and extracted three times with EA (30 ml). The organic phases were combined, washed with saturated brine, dried over anhydrous sodium sulfate, filtered, and concentrated to dryness. The residue was purified by silica gel column with eluent ratio of DCM/MeOH=100/1˜30/1 to afford the compound TPD12001-4 (130 mg, yellow solid, purity 97.218%), yield: 14.93%. LCMS (ESI) m/z calcd. for C₂₃H₃₂N₆O₅ [M+H]⁺ 473.3; found 473.3; ¹H NMR (400 MHz, DMSO_d₆): δ=11.04 (s, 1H), 8.15 (d, J=5.2 Hz, 1H), 6.82 (d, J=5.2 Hz, 1H), 6.67 (t, J=5.4 Hz, 1H), 5.12 (dd, J=13.3, 5.1 Hz, 1H), 4.27 (d, J=18.1 Hz, 1H), 4.27 (d, J=18.1 Hz, 1H), 4.15 (d, J=18.0 Hz, 1H), 3.52 (dd, J=13.1, 6.6 Hz, 2H), 2.96-2.84 (m, 2H), 2.64-2.54 (m, 2H), 2.39-2.29 (m, 8H), 2.08-2.03 (m, 2H), 1.39 (s, 9H). The compound structure is confirmed.

To a 25 ml single-necked vial was sequentially added compound TPD12001-4 (130 mg, 0.27 mmol), 1,4-dioxane (5 ml) and 4N HCl/1,4-dioxane (3 ml). The reaction was carried out at 25° C. for 3 h. After completion of the reaction, it was concentrated to dryness to give the crude compound TPD12001-5 (140 mg, yellow solid), yield: 94.68%. LCMS (ESI) m/z calcd. for C₁₈H₂₄N₆O₃ [M+H]⁺ 373.2; found 373.2. The compound structure is confirmed.

To a 25 ml single-necked vial were sequentially added compound TPD12001-5 (70 mg, 0.19 mmol), compound TPD5488-2 (110 mg, 0.19 mmol), sodium triacetoxyborohydride (80 mg, 0.38 mmol), and DCE (3 ml). The reaction was carried out under nitrogen protection at 25° C. for 16 hours. After the reaction was completed, the reaction solution was poured into water (15 ml) and an appropriate amount of saturated NaHCO₃ solution was added to make the reaction solution alkaline. It was extracted three times with DCM (10 ml). The organic phases were combined, washed with saturated brine, dried over anhydrous sodium sulfate, filtered, and concentrated to dryness. The residue was purified by preparative plate with unfolding agent ratio DCM/MeOH=8/1 to give compound TPD12001 (12.5 mg, light yellow solid, purity 96.23%), yield: 6.81%. LCMS (ESI) m/z calcd. for C₅₁H₅₅FN₁₀O₇ [M+H]⁺ 940.1; found 940.2; ¹H NMR (400 MHz, DMSO_d₆): δ=11.05 (s, 1H), 10.18 (s, 1H), 10.06 (s, 1H), 8.43 (d, J=5.2 Hz, 1H), 8.16 (d, J=5.1 Hz, 1H), 7.76 (d, J=8.9 Hz, 2H), 7.66-7.63 (m, 2H), 7.46 (s, 1H), 7.31 (s, 1H), 7.21 (d, J=9.0 Hz, 2H), 7.15 (t, J=8.9 Hz, 2H), 7.02 (s, 1H), 6.83 (d, J=5.2 Hz, 1H), 6.39 (d, J=5.2 Hz, 1H), 5.13 (dd, J=13.3, 5.1 Hz, 1H). 4.28 (d, J=18.0 Hz, 1H), 4.16 (d, J=17.9 Hz, 1H), 3.94 (s, 4H), 3.60-3.53 (m, 4H), 2.95-2.87 (m, 1H), 2.67-2.59 (m, 4H), 2.33-2.24 (m, 8H), 2.07-2.04 (m, 1H), 1.82 (d, J=11.6 Hz, 2H), 1.70 (s, 1H), 1.47 (s, 4H), 1.32 (d, J=14.9 Hz, 4H), 1.25 (d, J=3.8 Hz, 2H). The compound structure is confirmed.

### Example 59: Synthesis of Compound TPD12003

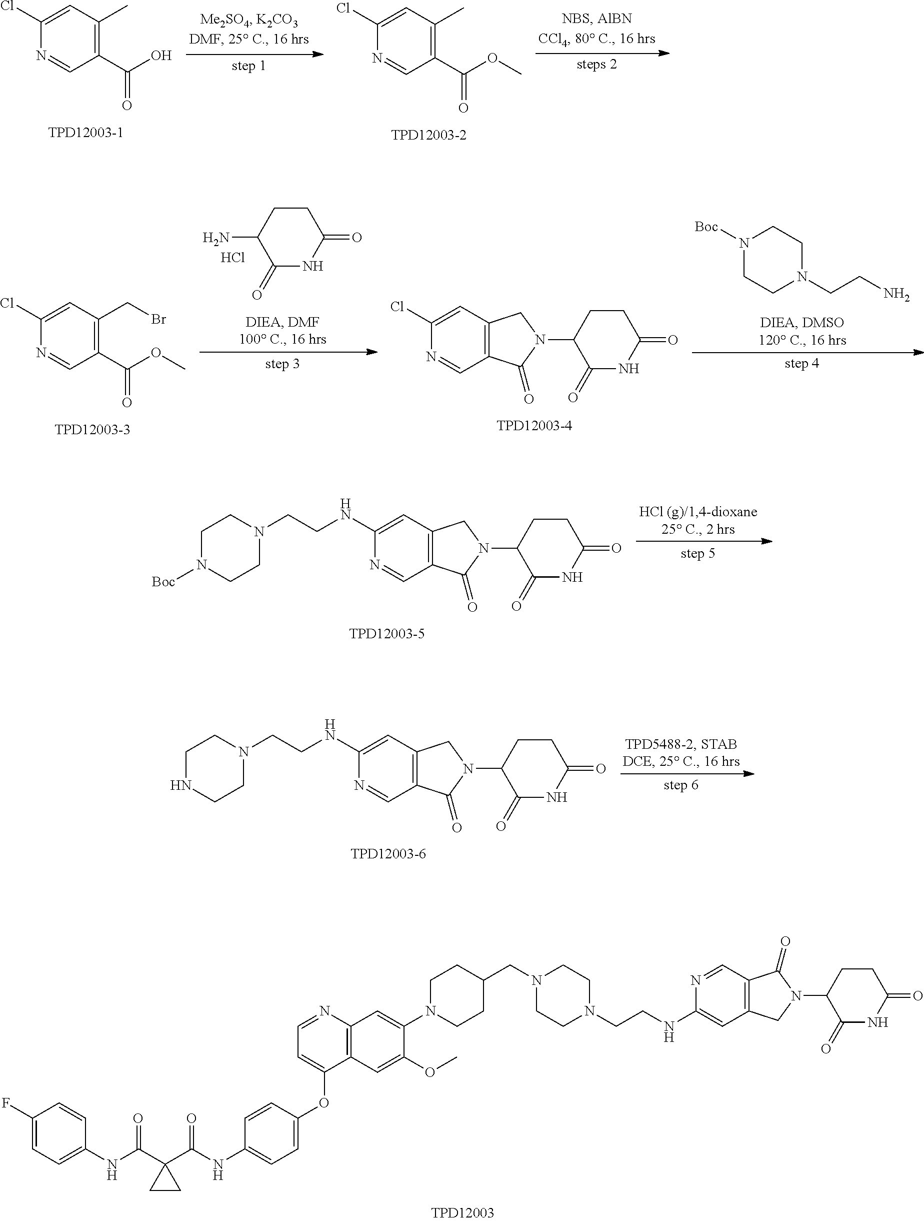

To a 100 ml triple-necked vial was sequentially added compound TPD12003-1 (3 g, 17.5 mmol), dimethyl sulfate (4.41 g, 35 mmol), K₂CO₃ (7.26 g, 52.5 mmol) and DMF (40 ml). The reaction was carried out under nitrogen protection at 25° C. for 16 hours. After the reaction was completed, the reaction solution was poured into water (150 ml) and extracted three times with EA (80 ml). The organic phases were combined, washed with saturated brine, dried over anhydrous sodium sulfate, filtered, and concentrated to dryness, and the residue was purified by silica gel chromatographic column with eluent ratio of PE/EtOAc=20/1˜5/1 to afford compound TPD12003-2 (2.5 g, white solid, purity 93.222%), yield: 61.76%. LCMS (ESI) m/z calcd. for C₈H₈ClNO₂ [M+H]⁺ 186.0; found 186.0; ¹H NMR (400 MHz, CDCl₃): δ=8.88 (s, 1H), 7.25 (s, 1H), 3.94 (s, 3H), 2.63 (s, 3H). The compound structure is confirmed.

Compound TPD12003-2 (1.5 g, 8.1 mmol), NBS (2.16 g, 12.2 mmol), AIBN (0.13 g, 0.81 mmol) and CCl₄ (30 ml) were added sequentially to a 100 ml three-necked vial. The reaction was carried out under nitrogen protection at 80° C. for 16 hours. After completion of the reaction, the reaction solution was brought to room temperature, decanted into water (60 ml) and extracted three times with DCM (20 ml). The organic phases were combined, washed with saturated brine, dried over anhydrous sodium sulfate, filtered, and concentrated to dryness to afford the crude compound TPD12003-3 (2.4 g, yellow oily substance), yield: 33.33%. LCMS (ESI) m/z calcd. for C₈H₇BrClNO₂ [M+H]⁺ 263.9, 265.9; found 263.9, 265.9. 265.9. The compound structure is confirmed.

To a 100 ml triple-necked vial were sequentially added compounds TPD12003-3 (2.4 g, 9.1 mmol), 3-amino-2,6-piperidinedione hydrochloride (1.8 g, 10.9 mmol), DIEA (2.35 g, 18.2 mmol) and DMF (30 ml). The reaction was carried out under nitrogen protection at 100° C. for 16 hours. After completion of the reaction, the reaction solution was reduced to room temperature, decanted into water (100 ml) and extracted three times with EA (50 ml). The organic phases were combined, washed with saturated brine, dried over anhydrous sodium sulfate, filtered, and concentrated to dryness. The residue was purified by silica gel column with eluent ratio of DCM/MeOH=100/1˜30/1 to give compound TPD12003-4 (500 mg, mauve solid, purity 99.318%), yield: 19.78%. LCMS (ESI) m/z calcd. for C₁₂H₁₀ClN₃O₃ [M+H]⁺ 280.0; found 280.1; ¹H NMR (400 MHz, DMSO_d₆): δ=11.04 (s, 1H), 8.80 (s, 1H), 7.87 (s, 1H), 5.14 (dd, J=13.3, 5.1 Hz, 1H), 4.57 (d, J=18.8 Hz, 1H), 4.44 (d, J=18.7 Hz, 1H). 2.96-2.86 (m, 1H), 2.63-2.57 (m, 1H), 2.46-2.35 (m, 1H), 2.04-2.00 (m, 1H). The compound structure is confirmed.

Compound TPD12003-4 (200 mg, 0.72 mmol), 4-N-(2-aminoethyl)-1-N-BOC-piperidine (197 mg, 0.86 mmol), DIEA (185 mg, 1.43 mmol), and DMSO (5 ml) were added sequentially in 25 ml triple vials. The reaction was carried out under nitrogen protection at 120° C. for 16 hours. After completion of the reaction, the reaction solution was reduced to room temperature, decanted into water (20 ml) and extracted three times with EA (10 ml). The organic phases were combined, washed with saturated brine, dried over anhydrous sodium sulfate, filtered, and concentrated to dryness. The residue was purified by silica gel column with eluent ratio of DCM/MeOH=100/1˜30/1 to give compound TPD12003-5 (100 mg, brown solid, purity 90.95%), yield: 26.86%. LCMS (ESI) m/z calcd. for C₂₃H₃₂N₆O₅ [M+H]⁺ 473.2; found 473.2; ¹H NMR (400 MHz, DMSO_d₆): δ=10.96 (s, 1H), 8.35 (s, 1H), 7.09 (s, 1H), 6.59 (s, 1H), 5.03 (dd, J=13.3, 5.1 Hz, 1H), 4.32 (d, J=17.4 Hz, 1H), 4.18 (d, J=17.5 Hz, 1H), 3.46-3.42 (m, 2H), 3.31 (br. s, 4H), 2.94-2.85 (m, 1H), 2.60-2.51 (m, 2H), 2.38-2.31 (m, 6H), 1.97-1.92 (m, 1H), 1.39 (s, 9H). The compound structure is confirmed.

Compound TPD12003-5 (100 mg, 0.21 mmol), 1,4-dioxane (1 ml) and 4N HCl/1,4-dioxane (2 ml) were added sequentially in a 25 ml single-necked vial. The reaction was carried out under nitrogen protection at 25° C. for 2 hours. After completion of the reaction it was concentrated to dryness to give the crude compound TPD12003-6 (100 mg, brown solid), yield: 95.50%. LCMS (ESI) m/z calcd. for C₁₈H₂₄N₆O₃ [M+H]⁺ 373.2; found 373.2. The compound structure is confirmed.

To a 25 ml single-necked vial was sequentially added compound TPD12003-6 (100 mg, 0.27 mmol), compound TPD5488-2 (156 mg, 0.27 mmol), sodium triacetoxyborohydride (114 mg, 0.54 mmol) and DCE (3 ml). The reaction was carried out under nitrogen protection at 25° C. for 16 hours. After the reaction was completed, the reaction solution was decanted into water (5 ml) and an appropriate amount of saturated NaHCO₃ solution was added to make the reaction solution alkaline. The organic phase was separated and the aqueous phase was extracted twice with DCM (5 ml). The organic phases were combined, washed with saturated brine, dried over anhydrous sodium sulfate, filtered, and concentrated to dryness. The residue was purified by preparative plate with unfolding agent ratio DCM/MeOH=6/1 to give compound TPD12003 (16.1 mg, yellow solid, purity 98.655%), yield: 6.29%. LCMS (ESI) m/z calcd. for C₅₁H₅₅FN₁₀O₇ [M+H]⁺ 939.4; found 939.4; ¹H NMR (400 MHz, DMSO_d₆): δ=10.97 (s, 1H), 10.19 (s, 1H), 10.06 (s, 1H), 8.43 (d, J=5.2 Hz, 1H), 8.36 (s, 1H), 7.76 (d, J=8.9 Hz, 2H), 7.66-7.63 (m, 2H), 7.46 (s, 1H). 7.31 (s, 1H), 7.21 (d, J=8.9 Hz, 2H), 7.15 (t, J=8.9 Hz, 2H), 7.08 (br. s, 1H), 6.60 (s, 1H), 6.39 (d, J=5.2 Hz, 1H), 5.03 (dd, J=13.2, 5.0 Hz, 1H), 4.33 (d, J=17.4 Hz, 1H), 4.18 (d, J=17.4 Hz, 1H), 3.94 (s, 3H), 3.59 (d, J=11.1 Hz, 2H), 3.44 (s, 2H), 2.95-2.85 (m, 1H), 2.67-2.56 (m, 4H), 2.38-2.28 (m, 6H), 2.20 (br. s, 2H), 1.96-1.91 (m, 1H), 1.82 (d, J=11.4 Hz, 2H), 1.70 (br. s, 1H), 1.47 (s, 4H), 1.34-1.23 (m, 6H). The compound structure is confirmed.

### Example 60: Synthesis of Compound TPD12009

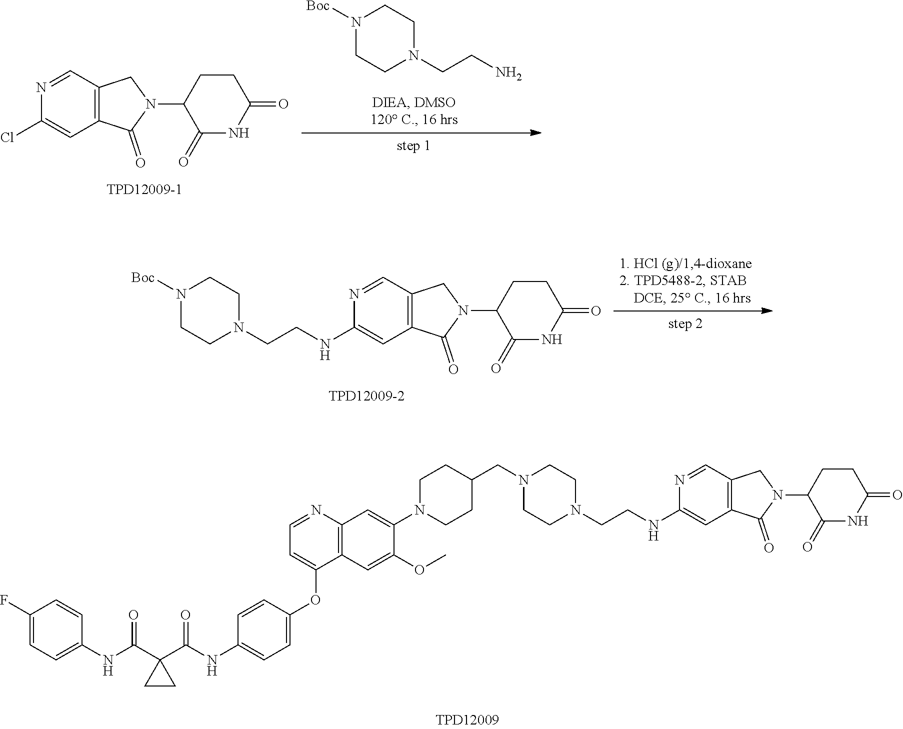 [SMILES: O=C1CCC(N2Cc3cnc(Cl)cc3C2=O)C(=O)N1 ; CC(C)(C)OC(=O)N1CCN(CCN)CC1 ; CC(C)(C)OC(=O)N1CCN(CCNc2cc3c(cn2)CN(C2CCC(=O)NC2=O)C3=O)CC1 ; COc1cc2c(Oc3ccc(NC(=O)C4(C(=O)Nc5ccc(F)cc5)CC4)cc3)ccnc2cc1N1CCC(CN2CCN(CCNc3cc4c(cn3)CN(C3CCC(=O)NC3=O)C4=O)CC2)CC1]

A 25 ml single-necked vial was charged sequentially with compound TPD12009-1 (50 mg, 0.18 mmol), tert-butyl 4-(2-aminoethyl)piperazine-1-carboxylate (62 mg, 0.27 mmol), DMSO (5 ml) and DIEA (69 mg, 0.54 mmol). The reaction was carried out at 140° C. for 16 h under nitrogen protection. The system was cooled to room temperature and poured into ice water (20 ml) and extracted three times with ethyl acetate (10 ml). The organic phases were combined and washed sequentially with water (10 ml) and saturated saline (10 ml). Anhydrous sodium sulfate was dried, filtered and concentrated to dryness. The residue was purified by preparative plate with unfolding agent DCM/MeOH=15/1 to afford the compound TPD12009-2 (10 mg, brown oil, purity 58.279%), yield: 6.88%. LCMS (ESI) m/z calcd. for C₂₃H₃₂N₆O₅ [M+H]⁺ 473.2; found 473.1. The compound structure is confirmed.

To a 40 ml single-necked vial was added compound TPD12009-2 (20 mg, 0.042 mmol), DCM (1 ml) and hydrogen chloride dioxane solution (2 ml, 4 M). The system was spun dry after 1 h of reaction at 30° C. DCE (0.5 ml) and sodium triacetoxyborohydride (18 mg, 0.084 mmol) were added. The reaction was carried out under nitrogen protection at 30° C. for 72 hours. The system was poured into saturated aqueous sodium bicarbonate solution (2 ml) and extracted three times with DCM (2 ml). The organic phases were combined and washed sequentially with water (2 ml) and saturated saline (2 ml). Anhydrous sodium sulfate was dried, filtered and concentrated to dryness. The residue was purified by preparative plate with unfolding agent DCM/MeOH=10/1 to give compound TPD12009 (2.4 mg, yellow solid, purity 83.866%), yield: 4.98%. LCMS (ESI) m/z calcd. for C₅₁H₅₅FN₁₀O₇ [M+H]⁺ 939.4; found 939.7; ¹H NMR (400 MHz, CD₃ OD): δ=8.38 (d, J=5.4 Hz, 1H), 8.24 (s, 1H), 7.71 (d, J=8.9 Hz, 2H), 7.60 (s, 1H), 7.58-7.54 (m, 3H), 7.47-7.42 (m, 1H), 7.39 (s, 1H), 7.22-7.19 (m, 2H), 7.09-7.05 (m, 3H), 6.88 (d, J=1.0 Hz, 1H), 6.50 (d, J=5.4 Hz, 1H), 5.34 (t, J=4.7 Hz, 2H), 5.41-5.27 (m, 1H), 4.02 (s, 3H), 3.69 (d, J=11.2 Hz, 2H), 3.49 (d, J=5.9 Hz, 2H), 3.13-3.10 (m, 1H), 2.78-2.73 (m, 4H), 2.59-2.55 (m, 2H), 2.39 (d, J=7.0 Hz, 2H), 2.21-2.17 (m, 2H), 2.02 (d, J=5.0 Hz, 2H), 1.92 (d, J=8.7 Hz, 2H), 1.80 (br. s, 2H), 1.64 (s, 4H), 1.33 (s, 6H). The compound structure is confirmed.

### Example 61: Synthesis of Compound TPD12013

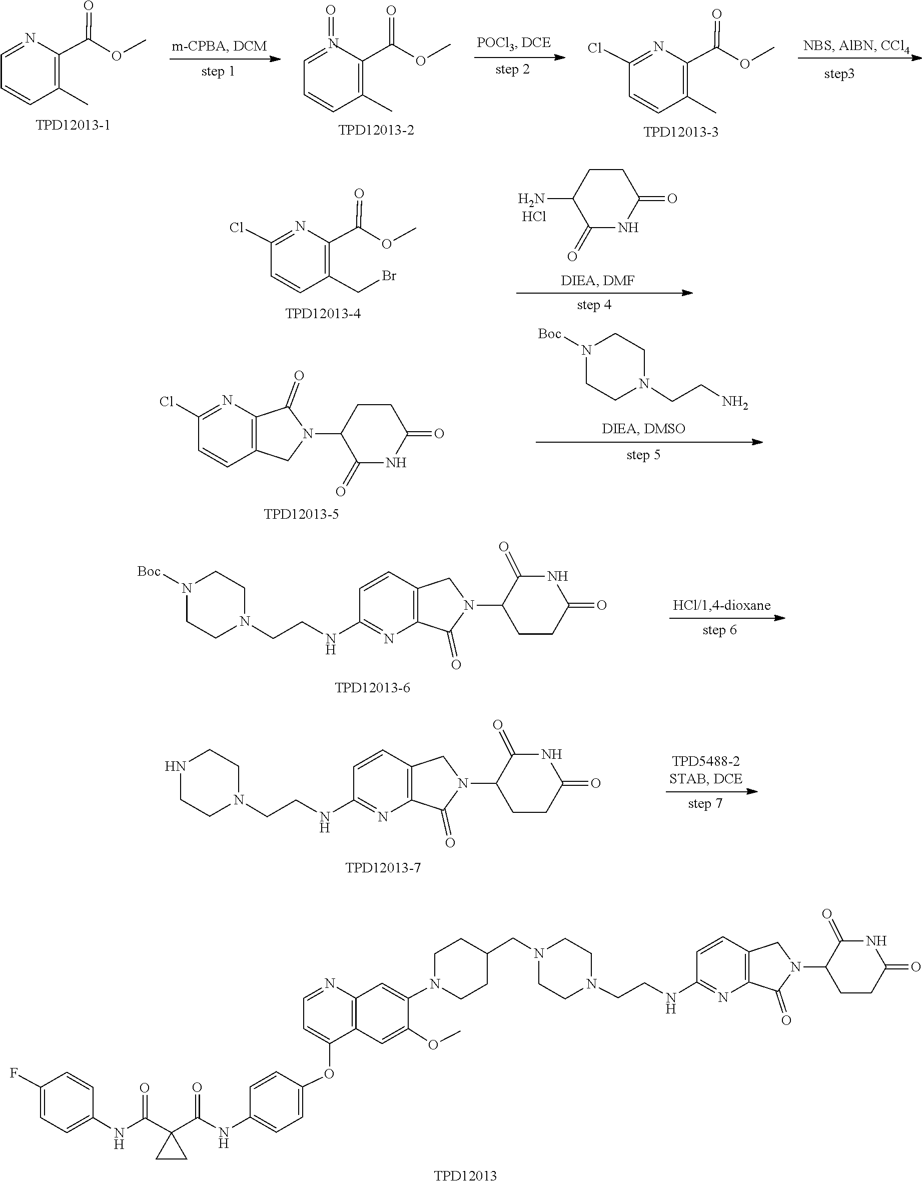

Compound TPD12013-1 (5.0 g, 33.1 mmol) and DCM (100 ml) were added sequentially in a 250 ml three-necked vial. The system was lowered to 0° C. and m-CPBA (8.57 g, 49.6 mmol) was added. The reaction was carried out under nitrogen protection at 25° C. for 16 hours. After completion of the reaction, the reaction solution was poured into saturated NaHCO₃ solution (100 ml) and extracted three times with DCM (50 ml). The organic phases were combined, washed with saturated brine, dried over anhydrous sodium sulfate, filtered, and concentrated to dryness, and the residue was purified by silica gel chromatographic column with eluent ratio of DCM/MeOH=100/1˜30/1 to obtain the compound TPD12013-2 (6 g, yellow oil, purity 81.067%), yield: 87.92%. LCMS (ESI) m/z calcd. for C₈H₉NO₃ [M+H]⁺ 168.1; found 168.1; ¹H NMR (400 MHz, CDCl₃): δ=8.13 (d, J=6.4 Hz, 1H), 7.24-7.20 (m, 1H), 7.15 (d, J=7.9 Hz, 1H), 4.03 (s, 3H), 2.31 (s, 3H). The compound structure is confirmed.

Compound TPD12013-2 (6.0 g, 35.9 mmol), POCl₃ (27.52 g, 179.5 mmol) and DCE (60 ml) were added sequentially in a 100 ml triple-necked vial. The reaction was carried out under nitrogen protection at 80° C. for 16 hours. After completion of the reaction, the reaction solution was reduced to room temperature, concentrated and adjusted to alkaline with saturated NaHCO₃ solution and extracted three times with DCM (30 ml). The organic phases were combined, washed with saturated brine, dried over anhydrous sodium sulfate, filtered, and concentrated to dryness, and the residue was purified by silica gel chromatographic column with eluent ratio of PE/EtOAc=20/1˜5/1 to obtain the compound TPD12013-3 (1.9 g, brown oil, purity 97.863%) in the yield: 27.86%. LCMS(ESI) m/z calcd. for C₈H₈ClNO₂ [M+H]⁺ 186.0; found 186.0; ¹H NMR (400 MHz, CDCl₃): δ=7.90 (d, J=1.8 Hz, 1H), 7.23 (d, J=5.2 Hz, 1H), 3.89 (s, 3H), 2.32 (s, 3H). The compound structure is confirmed.

Compound TPD12013-3 (1.7 g, 9.2 mmol), NBS (2.46 g, 13.8 mmol), AIBN (0.15 g, 0.92 mmol) and CCl₄ (50 ml) were added sequentially in a 100 ml triple-necked vial. The reaction was carried out under nitrogen protection at 80° C. for 16 hours. After completion of the reaction, the reaction solution was brought to room temperature, decanted into water (100 ml) and extracted three times with DCM (50 ml). The organic phases were combined, washed with saturated brine, dried over anhydrous sodium sulfate, filtered, and concentrated to dryness, and the residue was purified by silica gel chromatographic column with eluent ratio of PE/EtOAc=50/1˜30/1 to afford the compound TPD12013-4 (1.7 g, yellow oil, purity 97.915%) in the yield: 68.48%. LCMS (ESI) m/z calcd. for C8H₇BrClNO₂ [M+H]⁺ 263.9; found 264.0; ¹H NMR (400 MHz, CDCl₃): δ=8.54 (d, J=5.2 Hz, 1H), 7.54 (d, J=5.2 Hz, 1H), 5.04 (s, 2H), 4.05 (s, 3H). The compound structure is confirmed.

To a 100 ml triple-necked vial were sequentially added compounds TPD12013-4 (1.7 g, 6.4 mmol), 3-amino-2,6-piperidinedione hydrochloride (1.26 g, 7.68 mmol), DIEA (1.65 g, 12.8 mmol) and DMF (30 ml). The reaction was carried out under nitrogen protection at 100° C. for 16 hours. After completion of the reaction, the reaction solution was reduced to room temperature, decanted into water (100 ml) and extracted three times with EA (50 ml). The organic phases were combined, washed with saturated brine, dried over anhydrous sodium sulfate, filtered, and concentrated to dryness. The residue was purified by silica gel column with eluent ratio of DCM/MeOH=100/1˜30/1 to give compound TPD12013-5 (1 g, purple solid, purity 90.452%), yield: 50.00%. LCMS (ESI) m/z calcd. for C₁₂H₁₀ClN₃O₃ [M+H]⁺ 280.0; found 280.1; ¹H NMR (400 MHz, DMSO_d₆): δ=11.06 (s, 1H), 8.75 (d, J=5.3 Hz, 1H), 7.81 (d, J=5.3 Hz, 1H), 5.20 (dd, J=13.3, 5.1 Hz, 1H), 4.58 (d, J=17.8 Hz, 1H), 4.42 (d, J=17.8 Hz, 1H), 2.98-2.89 (m, 1H), 2.65-2.60 (m, 1H), 2.55-2.44 (m, 1H), 2.07-2.03 (m, 1H). The compound structure is confirmed.

Compound TPD12013-5 (350 mg, 1.25 mmol), 4-N-(2-aminoethyl)-1-N-BOC-piperidine (344.37 mg, 1.50 mmol), DIEA (323.49 mg, 2.50 mmol), and DMSO (5 ml) were added sequentially in 25 ml triple vials. The reaction was carried out under nitrogen protection at 120° C. for 16 hours. After completion of the reaction, the reaction solution was reduced to room temperature, decanted into water (10 ml) and extracted three times with EA (5 ml). The organic phases were combined, washed with saturated brine, dried over anhydrous sodium sulfate, filtered, and concentrated to dryness. The residue was purified by preparative plate with unfolding agent ratio DCM/MeOH=10/1 to give compound TPD12013-6 (240 mg, yellow solid, purity 57.007%), yield: 23.09%. LCMS (ESI) m/z calcd. for C₂₃H₃₂N₆O₅ [M+H]⁺ 473.2; found 473.3. The compound structure is confirmed.

Compound TPD12013-6 (240 mg, 0.5068 mmol), 1,4-dioxane (2 ml) and 4N HCl/1,4-dioxane (4 ml) were added sequentially in a 25 ml single-necked vial. The reaction was carried out under nitrogen protection at 25° C. for 2 hours. After completion of the reaction, it was concentrated to dryness to give the crude compound TPD12013-7 (240 mg, yellow solid), yield: 74.88%. LCMS (ESI) m/z calcd. for C₁₈H₂₄N₆O₃ [M+H]⁺ 373.2; found 373.2. The compound structure is confirmed.

To a 25 ml single-necked vial was sequentially added compound TPD12003-6 (100 mg, 0.27 mmol), compound TPD5488-2 (156 mg, 0.27 mmol), sodium triacetoxyborohydride (114 mg, 0.54 mmol) and DCE (3 ml). The reaction was carried out under nitrogen protection at 25° C. for 16 hours. After the reaction was completed, the reaction solution was decanted into water (5 ml) and an appropriate amount of saturated NaHCO₃ solution was added to make the reaction solution alkaline. The organic phase was separated and the aqueous phase was extracted twice with DCM (5 ml). The organic phases were combined, washed with saturated brine, dried over anhydrous sodium sulfate, filtered, and concentrated to dryness. The residue was purified by preparative plate with unfolding agent ratio DCM/MeOH=6/1 to afford compound TPD12003 (16.1 mg, yellow solid, purity 98.655%), yield: 6.29%. LCMS (ESI) m/z calcd. for C₅₁H₅₅FN₁₀O₇ [M+H]⁺ 939.4; found 470.2; ¹H NMR (400 MHz, DMSO_d₆): δ=11.04 (s, 1H), 10.19 (s, 1H), 10.06 (s, 1H), 8.43 (d, J=5.2 Hz, 1H), 8.25 (d, J=5.7 Hz, 1H), 7.76 (d, J=8.9 Hz, 2H), 7.66-7.62 (m, 2H), 7.46 (s, 1H), 7.31 (s, 1H), 7.21 (d, J=8.9 Hz, 2H), 7.17-7.13 (m, 2H), 6.68 (d, J=5.7 Hz, 1H), 6.57 (t, J=5.2 Hz, 1H), 6.39 (d, J=5.2 Hz, 1H), 5.13 (dd, J=13.3, 5.0 Hz, 1H), 4.25 (d, J=17.1 Hz, 1H), 4.14 (d, J=17.2 Hz, 1H), 3.94 (s, 3H), 3.59 (d, J=10.7 Hz, 4H), 3.32 (d, J=5.9 Hz, 2H), 2.97-2.88 (m, 1H), 2.67-2.61 (m, 4H). 2.42-2.26 (m, 6H), 2.20 (d, J=6.8 Hz, 2H), 2.07-1.99 (m, 1H), 1.82 (d, J=12.1 Hz, 2H), 1.69 (br. s, 1H), 1.47 (s, 4H), 1.36-1.24 (m, 4H). The compound structure is confirmed.

### Example 62: Synthesis of Compound TPD12068

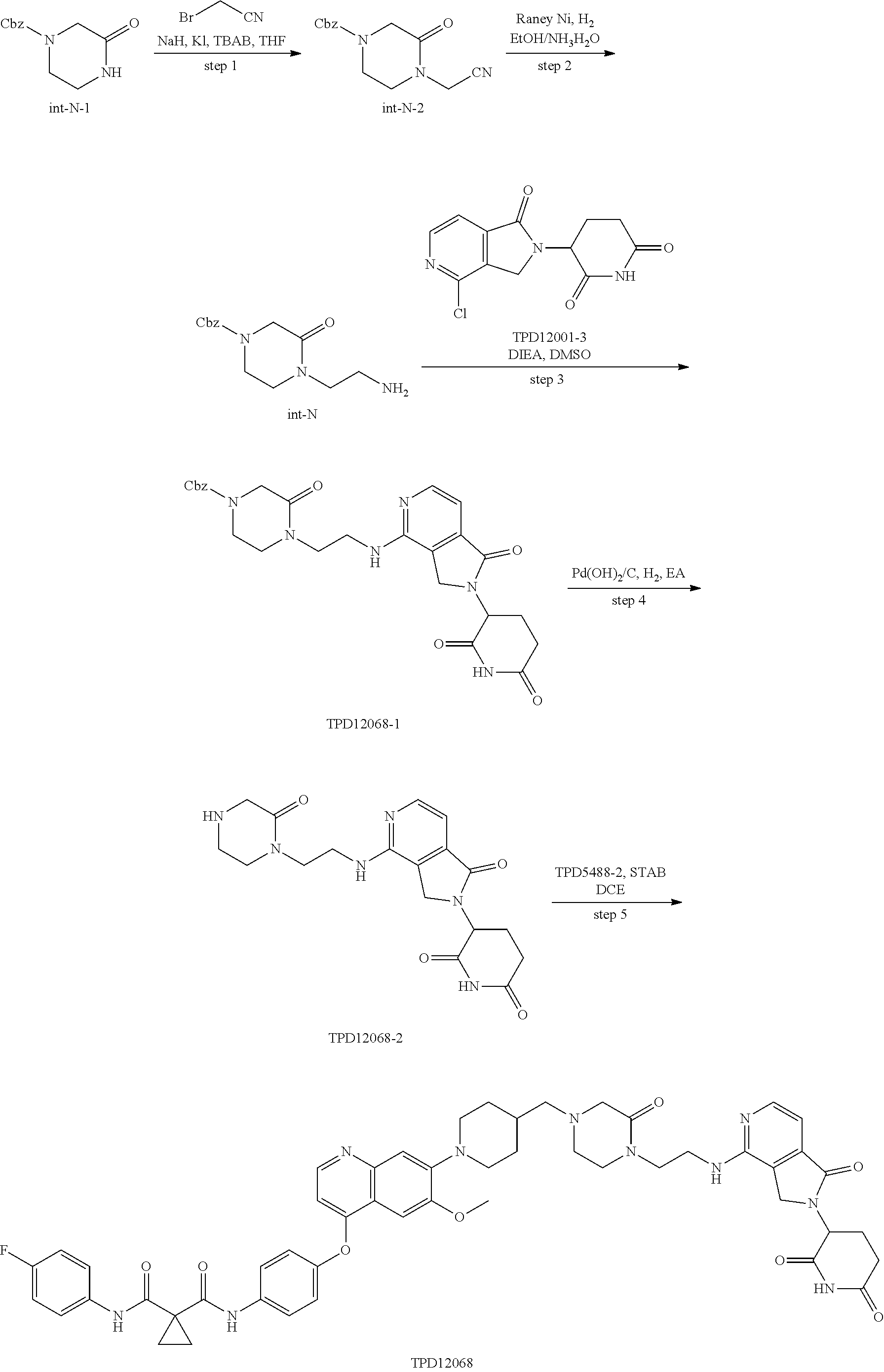

Compound int-N-1 (5.0 g, 21.3 mmol) and THF (100 ml) were added sequentially in a 250 ml triple-necked vial. NaH (1.70 g, 42.6 mmol) was added after the system was lowered to 0° C. The reaction was carried out under nitrogen protection at 0° C. for 1 hour. Then bromoacetonitrile (5.11 g, 42.6 mmol), potassium iodide (0.35 g, 2.13 mmol) and tetrabutylammonium bromide (0.69 g, 2.13 mmol) were added sequentially. The reaction was carried out under nitrogen protection at 25° C. for 15 hours. After the reaction was completed, the reaction solution was poured into saturated NH₄ C1 solution (300 ml) and extracted three times with EA (100 ml). The organic phases were combined, washed with saturated brine, dried over anhydrous sodium sulfate, filtered, and concentrated to dryness, and the residue was purified by silica gel chromatographic column with the eluent ratio of DCM/MeOH=100/1˜30/1 to obtain the compound int-N-2 (4.7 g, brown solid, purity 92.512%) in the yield: 74.65%. LCMS (ESI) m/z calcd. for C₁₄H₁₅N₃O₃ [M+H]⁺ 274.1; found 274.1; ¹H NMR (400 MHz, DMSO_d₆): δ=7.39-7.32 (m, 5H), 5.11 (s, 2H), 4.46 (s, 2H), 4.08 (s, 2H), 3.69 (s, 2H), 3.47 (t, J=5.5 Hz, 2H). The compound structure is confirmed.

To a 100 ml single-necked flask were added sequentially the compounds int-N-2 (5.0 g, 18.3 mmol), EtOH/NH H₃₂ O=10/1 (50 ml) and Raney nickel (1.07 g, 18.3 mmol). The reaction was carried out under hydrogen (1 atm) atmosphere for 16 hours at 25° C. After the reaction was completed, the reaction solution was filtrated, the filtrate was concentrated to dryness and the residue was purified by silica gel chromatographic column with eluent ratio of DCM/MeOH=50/1˜30/1 to obtain the compound int-N (2.0 g, brown oily substance, purity 94.845%), yield: 37.16%. LCMS (ESI) m/z calcd. for C₁₄H₁₉N₃O₃ [M+H]⁺ 278.1; found 278.2. The compound structure is confirmed.

Compound TPD12001-3 (670 mg, 2.40 mmol), int-N (664 mg, 2.40 mmol), DIEA (619 mg, 4.79 mmol) and DMSO (10 ml) were added sequentially in a 25 ml single-necked vial. The reaction was carried out under nitrogen protection at 145° C. for 16 hours. After completion of the reaction, the reaction solution was reduced to room temperature, decanted into water (60 ml) and extracted three times with EA (20 ml). The organic phases were combined, washed with saturated brine, dried over anhydrous sodium sulfate, filtered, and concentrated to dryness. The residue was purified by silica gel column with eluent ratio of DCM/MeOH=100/1˜30/1 to afford the compound TPD12068-1 (100 mg, brown solid, purity 93.032%), yield: 7.46%. LCMS (ESI) m/z calcd. for C₂₆H₂₈N₆O₆ [M+H]⁺ 521.2; found 521.3; ¹H NMR (400 MHz, DMSO_d₆): δ=11.04 (s, 1H), 8.14 (d, J=5.1 Hz, 1H), 7.38-7.35 (m, 5H), 6.89 (t, J=5.0 Hz, 1H), 6.83 (d, J=5.2 Hz, 1H), 5.09 (s, 2H), 4.24 (d J=17.9 Hz, 1H), 4.13 (d, J=17.9 Hz, 1H), 3.94 (s, 2H), 3.58-3.52 (m, 7H), 3.42-3.39 (m, 2H), 2.95-2.86 (m, 1H), 2.61 (d, J=16.4 Hz, 1H), 2.35-2.24 (m, 1H). 2.07-2.02 (m, 1H). The compound structure is confirmed.

Compound TPD 12068-1 (100 mg, 0.38 mmol), EA (5 ml) and Pd(OH)₂/C (54 mg, 10% wet) were added sequentially in a 25 ml single-necked vial. The reaction was carried out under hydrogen (1 atm) atmosphere at 40° C. for 48 hours. After completion of the reaction, the reaction solution was filtered and concentrated to dryness to afford the crude compound TPD12068-2 (30 mg, yellow oil, purity 92.378%), yield: 21.42%. LCMS (ESI) m/z calcd. for C₁₈H₂₂N₆O₄ [M+H]⁺ 387.2; found 387.2. The compound structure is confirmed.

To a 25 ml single-necked vial was sequentially added compound TPD12068-2 (30 mg, 0.078 mmol), compound TPD5488-2 (54 mg, 0.093 mmol), sodium triacetoxyborohydride (33 mg, 0.16 mmol), acetic acid (14 mg, 0.23 mmol) and DCE (3 ml). The reaction was carried out under nitrogen protection for 6 hours at 25° C. After the reaction was completed, the reaction solution was decanted into water (15 ml) and an appropriate amount of saturated NaHCO₃ solution was added to make the reaction solution alkaline. The organic phase was separated and the aqueous phase was extracted twice with DCM (5 ml). The organic phases were combined, washed with saturated brine, dried over anhydrous sodium sulfate, filtered, and concentrated to dryness. The residue was prepared by high performance liquid chromatography (HPLC) with the following parameters: column: sunfire 5 μm 19-150 mm; mobile phase: acetonitrile-water (0.1% FA); gradient: 15-40/6 min to afford the compound TPD12068 (8.62 mg, yellow solid, purity 98.240%) in the following yield: 11.08%. LCMS (ESI) m/z calcd. for C₅₁H₅₃FN₁₀O₈ [M+H]⁺ 953.4; found 477.3; ¹H NMR (400 MHz, DMSO_d₆): δ=11.06 (s, 1H), 10.18 (s, 1H), 10.06 (s, 1H), 8.43 (d, J=5.1 Hz, 1H), 8.22 (s, 0.6H). 8.17 (d, J=5.1 Hz, 1H), 7.76 (d, J=8.8 Hz, 2H), 7.64 (dd, J=8.8, 5.1 Hz, 2H), 7.46 (s, 1H), 7.32 (s, 1H), 7.21 (d, J=8.8 Hz, 2H), 7.15 (t, J=8.9 Hz, 2H), 6.87 (d, J=5.8 Hz, 1H), 6.84 (d, J=5.1 Hz, 1H), 6.39 (d, J=5.1 Hz, 1H), 5.12 (dd, J=13.2, 4.8 Hz, 1H), 4.26 (d, J=18.0 Hz, 1H), 4.15 (d, J=18.0 Hz, 1H), 3.94 (s, 3H), 3.58 (d, J=8.0 Hz, 5H), 3.50 (d, J=3.6 Hz, 3H), 2.97 (s, 2H), 2.93-2.87 (m, 1H), 2.67-2.60 (m, 5H), 2.36-2.29 (m, 1H), 2.24 (d, J=6.6 Hz, 2H), 1.80 (d, J=11.9 Hz, 2H), 1.69 (s, 1H), 1.47 (s, 4H), 1.35-1.23 (m, 3H). The compound structure is confirmed.

### Example 63: Synthesis of Compound TPD12081

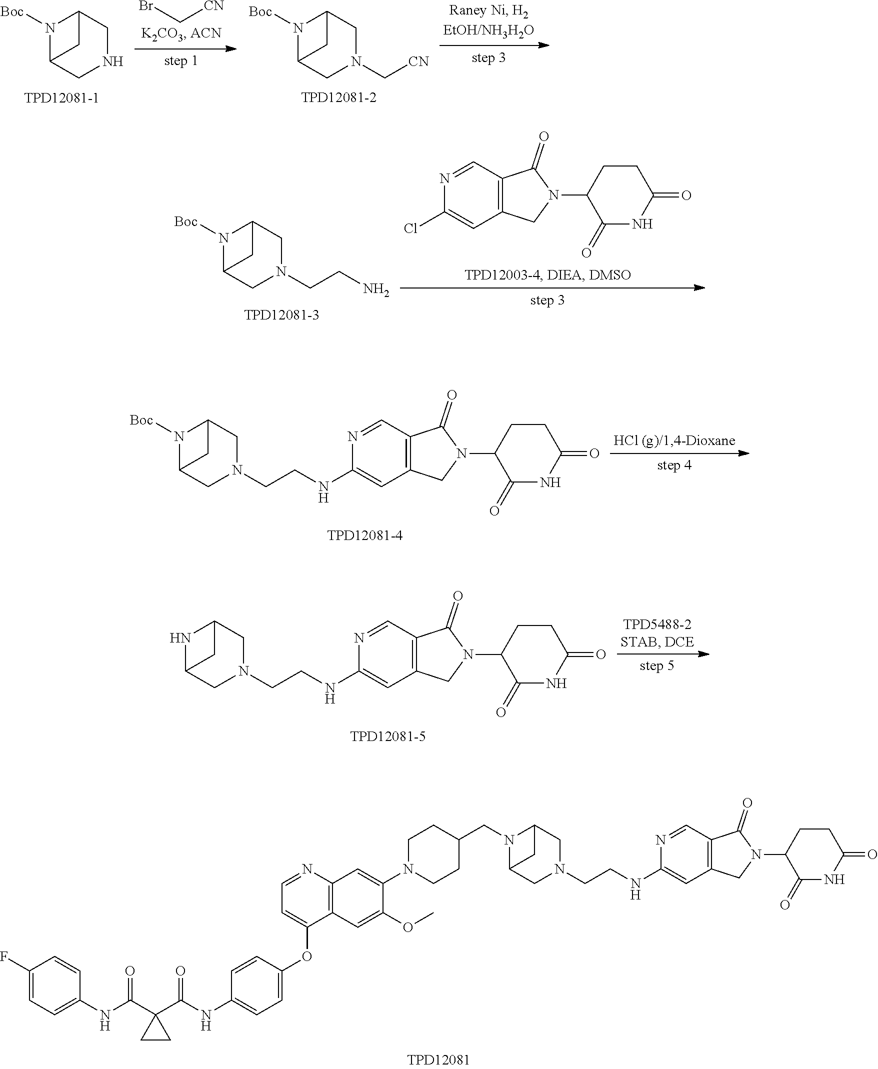

To a 100 ml triple-necked vial was sequentially added compound TPD12081-1 (2 g, 10.09 mmol), acetonitrile (30 ml), bromoacetonitrile (1.21 g, 10.09 mmol) and potassium carbonate (2.07 g, 15.13 mmol). The reaction was carried out under nitrogen protection at 25° C. for 4 hours. After the reaction was completed, the reaction solution was filtered, the filtrate was concentrated to dryness, and the residue was purified by silica gel chromatographic column with eluent ratio of DCM/MeOH=50/1˜30/1 to obtain the compound TPD12081-2 (1.6 g, yellow solid, purity 98.554%), yield: 66.00%. LCMS (ESI) m/z calcd. for C₁₂H₁₉N₃O₂ [M+H]⁺ 238.2; found 237.8; ¹H NMR (400 MHz, CDCl₃): δ=4.15-4.09 (m, 2H), 3.60 (s, 2H), 3.38-3.25 (m, 2H), 2.83-2.81 (m, 2H), 2.47-2.44 (m, 1H), 1.61-1.58 (m, 1H), 1.46 (s, 9H). The compound structure is confirmed.

To a 50 ml single-necked vial were sequentially added compound TPD12081-2 (1 g, 4.21 mmol), EtOH (10 ml), ammonia (1 ml) and Raney nickel (0.49 g). The reaction was carried out under 1 atm hydrogen at 25° C. for 16 hours. After the reaction was completed, the filtrate was withdrawn and concentrated to dryness and the residue was purified by silica gel column with eluent ratio of DCM/MeOH=50/1˜10/1 to obtain the compound TPD12081-3 (0.26 g, brown solid, purity 86.928%), yield: 21.43%. LCMS (ESI) m/z calcd. for C₁₂H₂₃FN₃O₂ [M+H]⁺ 242.18; found 242.2; ¹H NMR (400 MHz, DMSO_d₆): δ=4.00-3.99 (m, 2H), 3.80 (s, 2H), 3.15-3.06 (m, 3H), 2.75-2.65 (m, 3H), 2.34-2.27 (m, 2H), 1.55-1.53 (m, 2H), 1.39 (s, 9H). The compound structure is confirmed.

To a 25 ml single-necked vial were sequentially added compound TPD12081-3 (260 mg, 1.07 mmol), compound TPD12003-4 (330 mg, 1.18 mmol), DIEA (416 mg, 3.22 mmol) and DMSO (3 ml). The reaction was carried out under nitrogen protection at 120° C. for 16 hours. The reaction was completed the reaction solution was reduced to room temperature. The reaction solution was poured into water (10 ml) and the aqueous phase was extracted three times with ethyl acetate (10 ml). The organic phases were combined, washed with saturated brine, dried over anhydrous sodium sulfate, filtered, and concentrated to dryness, and the residue was purified by preparative plate with the unfolding agent ratio of DCM/MeOH=15/1 to afford the compound TPD12081-4 (100 mg, brown solid, purity 95.665%), yield: 18.37%. LCMS (ESI) m/z calcd. for C₂₄H₃₂N₆O₅ [M+H]⁺ 485.24; found 485.1; ¹H NMR (400 MHz, DMSO_d₆): δ=10.96 (br. s, 1H), 8.35 (s, 1H), 7.04 (s, 1H), 6.56 (s, 1H), 5.02 (dd, J=13.3, 5.1 Hz, 1H), 4.25 (dd. J=58.9, 17.4 Hz, 2H), 3.95 (d, J=5.9 Hz, 2H), 3.50-3.41 (m, 2H), 3.06-2.76 (m, 7H), 2.68 (t, J=6.7 Hz, 2H), 2.36-2.23 (m, 3H), 2.09-1.84 (m, 2H), 1.58 (d, J=7.8 Hz, 1H), 1.38 (s, 9H).

To a 25 ml single-necked vial was sequentially added compound TPD12081-4 (50 mg, 0.103 mmol) and HCl(g)/1.4-dioxane (40 ml, 4N). the reaction was carried out for half an hour at 25° C. The reaction solution was concentrated to dryness to give the crude compound TPD12081-5 (40 mg, yellow solid), yield: 90%. The reaction solution was concentrated to dryness to give the crude compound TPD12081-5 (40 mg, yellow solid), yield: 90%. LCMS (ESI) m/z calcd. for C₂₄H₃₃N₅O₅ [M+H]⁺ 385.2; found 385.1. The compound structure is confirmed.

To a 25 ml single-necked vial were sequentially added compound TPD5488-2 (100 mg, 0.18 mmol), DCE (4 ml), compound TPD12081-5 (69 mg, 0.18 mmol) and sodium triacetoxyborohydride (76 mg, 0.36 mmol). The reaction was carried out under nitrogen protection at 25° C. for 16 hours. After the reaction was completed, the reaction solution was poured into saturated aqueous sodium bicarbonate solution (10 ml) and the aqueous phase was extracted three times with DCM (10 ml). The organic phases were combined, washed with saturated brine, dried over anhydrous sodium sulfate, filtered, and concentrated to dryness, and the residue was purified by preparative plate with the unfolding agent ratio of DCM/MeOH=10/1 to afford the compound TPD12081 (15.3 mg, yellow solid, purity 99.365%), yield: 8.90%. LCMS (ESI) m/z calcd. for C₅₂H₅₅FN₁₀O₇ [M+H⁺ 951.4; found 952.4; ¹H NMR (400 MHz, CD₃ OD): δ=8.56-8.32 (m, 3H), 7.72 (d, J=8.9 Hz, 2H), 7.62 (s, 1H), 7.59-7.53 (m, 2H), 7.39 (s, 1H), 7.27-7.18 (m, 2H), 7.12-7.03 (m, 2H), 6.65 (s, 1H), 6.52 (d, J=5.4 Hz, 1H), 5.05 (dd, J=13.0, 4.7 Hz, 1H), 4.48-4.30 (m, 3H), 4.02 (s, 3H), 3.71 (br. s, 2H), 3.65-3.56 (m 2H), 3.51-3.32 (m, 4H), 3.16-2.87 (m, 4H), 2.85-2.60 (m, 4H), 2.33 (br. s, 2H), 2.11-1.78 (m, 4H), 1.68-1.46 (m, 6H). The compound structure is confirmed.

### Example 64: Synthesis of Compound TPD12082

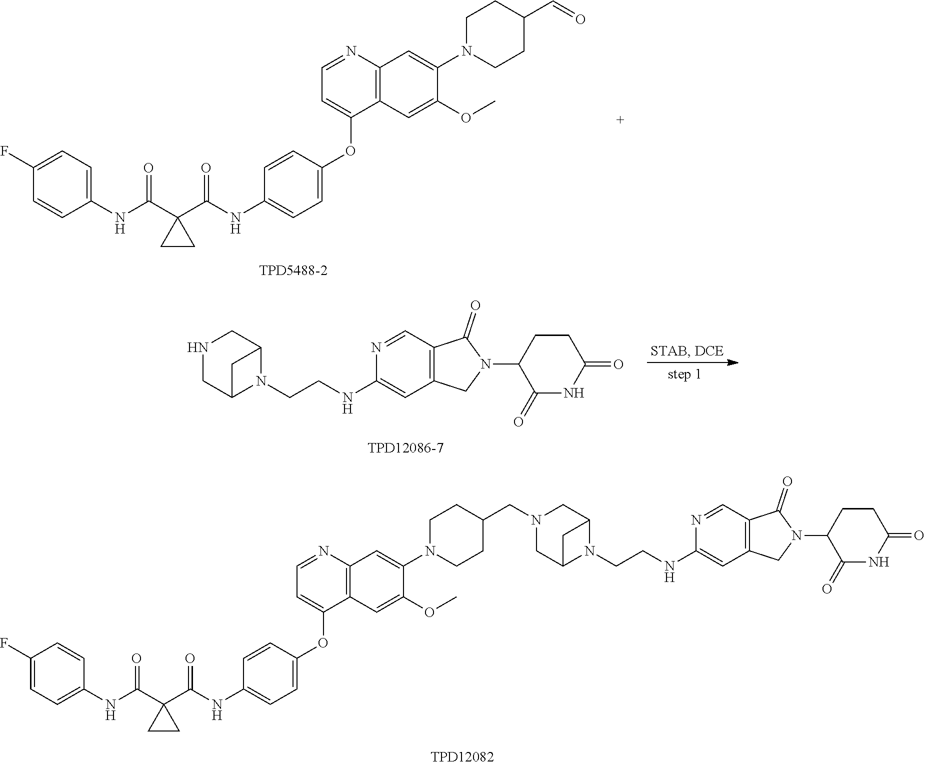 [SMILES: COc1cc2c(Oc3ccc(NC(=O)C4(C(=O)Nc5ccc(F)cc5)CC4)cc3)ccnc2cc1N1CCC(C=O)CC1 ; O=C1CCC(N2Cc3cc(NCCN4C5CNCC4C5)ncc3C2=O)C(=O)N1 ; COc1cc2c(Oc3ccc(NC(=O)C4(C(=O)Nc5ccc(F)cc5)CC4)cc3)ccnc2cc1N1CCC(CN2CC3CC(C2)N3CCNc2cc3c(cn2)C(=O)N(C2CCC(=O)NC2=O)C3)CC1]

To a 25 ml single-necked vial was sequentially added compound TPD12086-7 (50 mg, 0.13 mmol), compound TPD5488-2 (76 mg, 0.13 mmol), sodium triacetoxyborohydride (55 mg, 0.26 mmol) and DCE (3 ml). The reaction was carried out under nitrogen protection at 25° C. for 16 hours. After the reaction was completed, the reaction solution was decanted into water (15 ml) and an appropriate amount of saturated NaHCO₃ solution was added to make the reaction solution alkaline. The organic phase was separated and the aqueous phase was extracted twice with DCM (10 ml). The organic phases were combined, washed with saturated brine, dried over anhydrous sodium sulfate, filtered, and concentrated to dryness. The residue was prepared by high performance liquid chromatography (HPLC) with the following parameters: column: sunfire 5 μm 19-150 mm; mobile phase: acetonitrile-water (0.1% FA); gradient: 5-40/8 min to afford compound TPD12082 (9.44 mg, yellow solid, purity 98.466%), yield: 7.07%. LCMS (ESI) m/z calcd. for C₅₂H₅₅FN₁₀O₇ [M+H]⁺ 951.4; found 951.5; ¹H NMR (400 MHz, DMSO_d₆): δ=10.98 (s, 1H), 10.19 (s, 1H), 10.07 (s, 1H), 8.43 (d, J=5.1 Hz, 1H), 8.37 (s, 1H), 8.22 (s, 1H), 7.76 (d, J=8.6 Hz, 2H), 7.64 (dd, J=8.4, 5.2 Hz, 2H), 7.46 (s, 1H), 7.32 (s, 1H), 7.25-7.13 (m, 5H), 6.59 (s, 1H), 6.39 (d, J=5.1 Hz, 1H), 5.03 (dd, J=13.1 5.1 Hz, 1H), 4.33 (d, J=17.2 Hz, 1H), 4.19 (d, J=17.3 Hz, 1H), 3.94 (s, 3H), 3.66 (s, 3H), 2.97-2.84 (m, 6H), 2.67-2.57 (m, 4H), 2.36-2.26 (m, 4H), 1.98-1.79 (m, 5H), 1.70 (s, 1H), 1.47 (s, 4H), 1.35-1.23 (m, 4H). The compound structure is confirmed.

### Example 65: Synthesis of Compound TPD12083

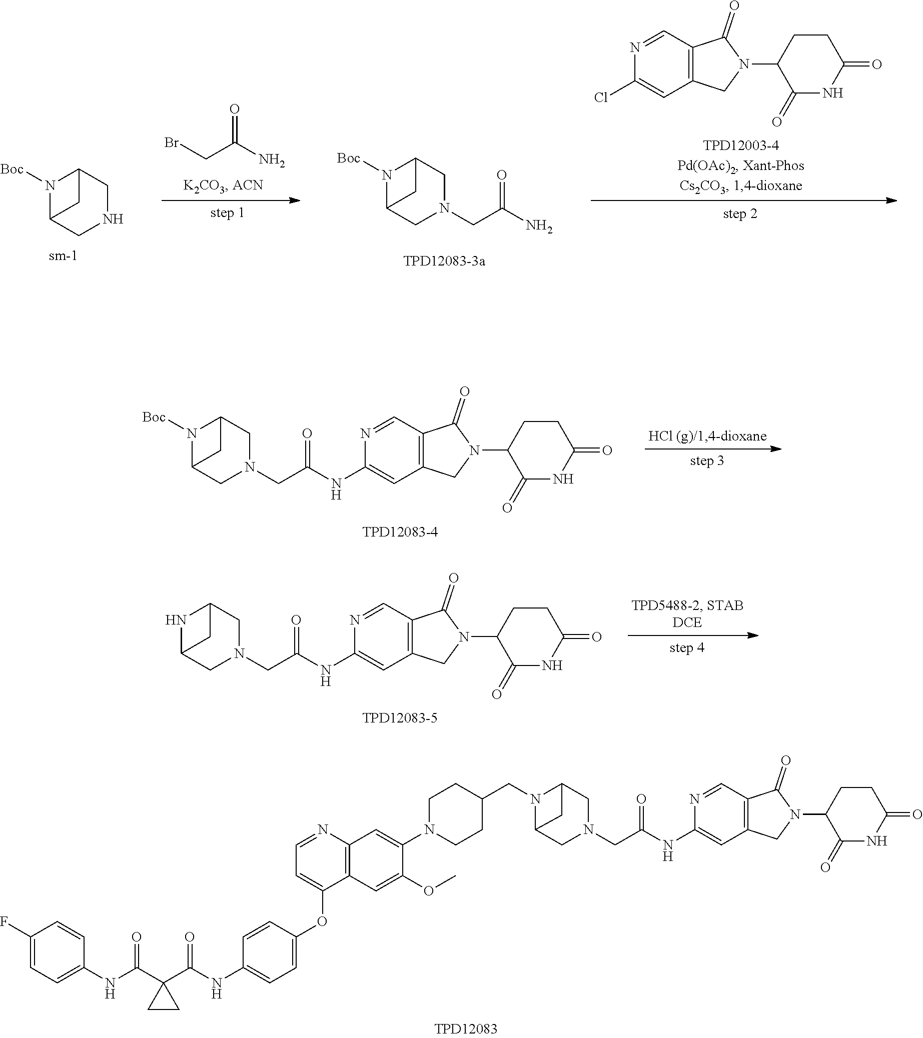

To a 50 ml single-necked vial was sequentially added compound sm-1 (500 mg, 2.51 mmol), 2-bromoacetamide (415 mg, 3.01 mmol), potassium carbonate (1040 mg, 7.53 mmol) and acetonitrile (10 ml). The reaction was carried out under nitrogen protection at 25° C. for 2 hours. After the reaction was completed, the reaction solution was poured into water (30 ml) and extracted three times with EA (30 ml). The organic phases were combined, washed with saturated brine, dried over anhydrous sodium sulfate, filtered, and concentrated to dryness, and the residue was purified by silica gel chromatographic column with eluent ratio of DCM/MeOH=100/1˜30/1 to afford the compound TPD12083-3a (500 mg, white solid, purity 96.019%) in the yield: 74.65%. LCMS (ESI) m/z calcd. for C₁₂H₂₁N₃O₃ [M+H]⁺ 256.2; found 255.7; ¹H NMR (400 MHz, DMSO_d₆): δ=7.16 (s, 1H), 7.01 (s, 1H), 3.94 (d, J=5.7 Hz, 2H), 3.10-2.99 (m, 4H), 2.80 (dd, J=41.5. 9.0 Hz, 2H), 2.30 (dd, J=13.7, 6.4 Hz, 1H), 1.59 (d, J=8.0 Hz, 1H), 1.38 (s, 9H). The compound structure is confirmed.

To a 25 ml single-necked vial was sequentially added compound TPD12083-3a (200 mg, 0.78 mmol), compound TPD12003-4 (218 mg, 0.78 mmol), palladium acetate (35 mg, 0.156 mmol), Xant-Phos (90 mg, 0.156 mmol), cesium carbonate (381 mg. 1.17 mmol) and 1,4-dioxane (5 ml). The reaction was carried out under nitrogen protection at 100° C. for 16 hours. After completion of the reaction, the reaction solution was brought to room temperature, decanted into water (30 ml) and extracted three times with EA (30 ml). The organic phases were combined, washed with saturated brine, dried over anhydrous sodium sulfate, filtered, and concentrated to dryness. The residue was purified by preparative plate with eluent ratio of DCM/MeOH=15/1 to afford the compound TPD12083-4 (57 mg, brown solid, purity 94.190%), yield: 13.78%. LCMS (ESI) m/z calcd. for C₂₄H₃₀N₆O [M+H]⁺ 499.2; found 499.2. The compound structure is confirmed.

To a 25 ml single-necked vial was sequentially added compound TPD12083-4 (57 mg, 0.114 mmol), 1,4-dioxane (2 ml) and 4N HCl/1,4-dioxane (2 ml). The reaction was carried out under nitrogen protection at 25° C. for 2 hours. After completion of the reaction it was concentrated to dryness to give the crude compound TPD12083-5 (60 mg, white solid), yield: 89.48%. LCMS (ESI) m/z calcd. for C₁₉H₂₂N₆O₄ [M+H]⁺ 399.2; found 399.2. The compound structure is confirmed.

To a 25 ml single-necked vial was sequentially added compound TPD12083-5 (60 mg, 0.15 mmol), compound TPD5488-2 (88 mg, 0.15 mmol), sodium triacetoxyborohydride (64 mg, 0.30 mmol) and DCE (3 ml). The reaction was carried out under nitrogen protection at 25° C. for 6 hours. After the reaction was completed, the reaction solution was decanted into water (15 ml) and an appropriate amount of saturated NaHCO₃ solution was added to make the reaction solution alkaline. The organic phase was separated and the aqueous phase was extracted twice with DCM (5 ml). The organic phases were combined, washed with saturated brine, dried over anhydrous sodium sulfate, filtered, and concentrated to dryness. The residue was prepared by high performance liquid chromatography (HPLC) with the following parameters: column: T3 5 μm 19-150 mm; mobile phase: acetonitrile-water (0.1% FA); gradient: 20-35/7 min. to obtain the compound TPD12083 (6.86 mg, yellow solid, purity 97.191%) in the following yield: 4.38%. LCMS (ESI) m/z calcd. for C₅₂H₅₃FN₁₀O₈ [M+H]⁺ 965.4; found 965.1; ¹H NMR (400 MHz, DMSO_d₆): δ=11.02 (s, 1H), 10.31 (s, 1H), 10.01 (s, 1H), 8.74-8.68 (m, 2H), 8.34 (d, J=8.0 Hz, 1H). 7.84 (d, J=8.8 Hz, 2H), 7.65-7.62 (m, 4H), 7.42 (s, 1H), 7.32 (d, J=8.5 Hz, 2H), 7.16 (t, J=8.9 Hz, 3H), 6.69 (br. s, 1H), 5.11 (dd, J=13.0, 4.8 Hz, 1H), 4.55 (d J=18.2 Hz, 1H), 4.41 (d, J=18.4 Hz, 1H), 4.04 (s, 3H), 3.77 (br. s, 4H), 3.65-3.56 (m, 3H), 2.81 (br. s, 3H), 2.67-2.57 (m, 1H), 2.33-2.25 (m, 5H), 2.03-1.91 (m, 6H), 1.62 (s, 1H), 1.48 (d, J=16.1 Hz, 8H). The compound structure is confirmed.

### Example 66: Synthesis of compound TPD12086

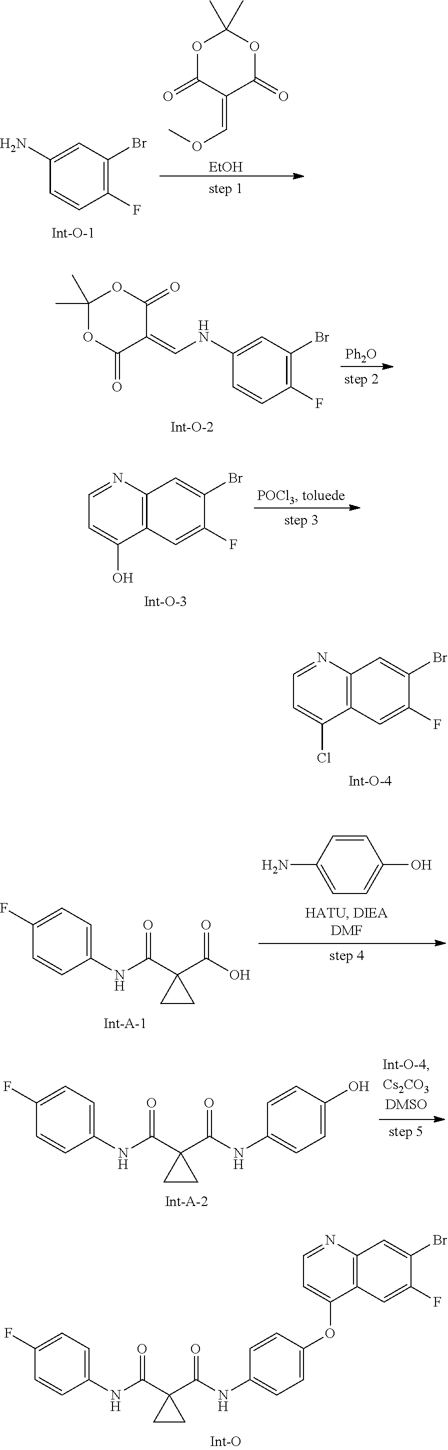

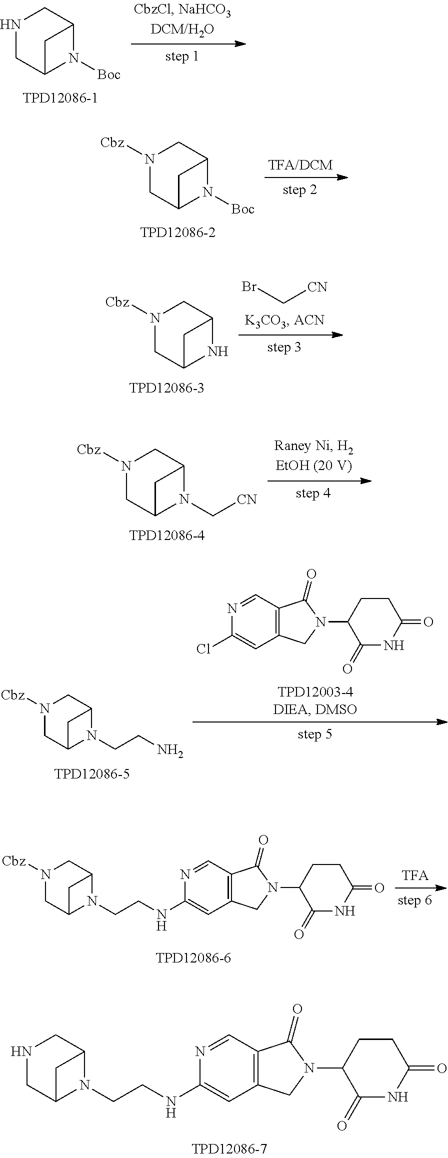

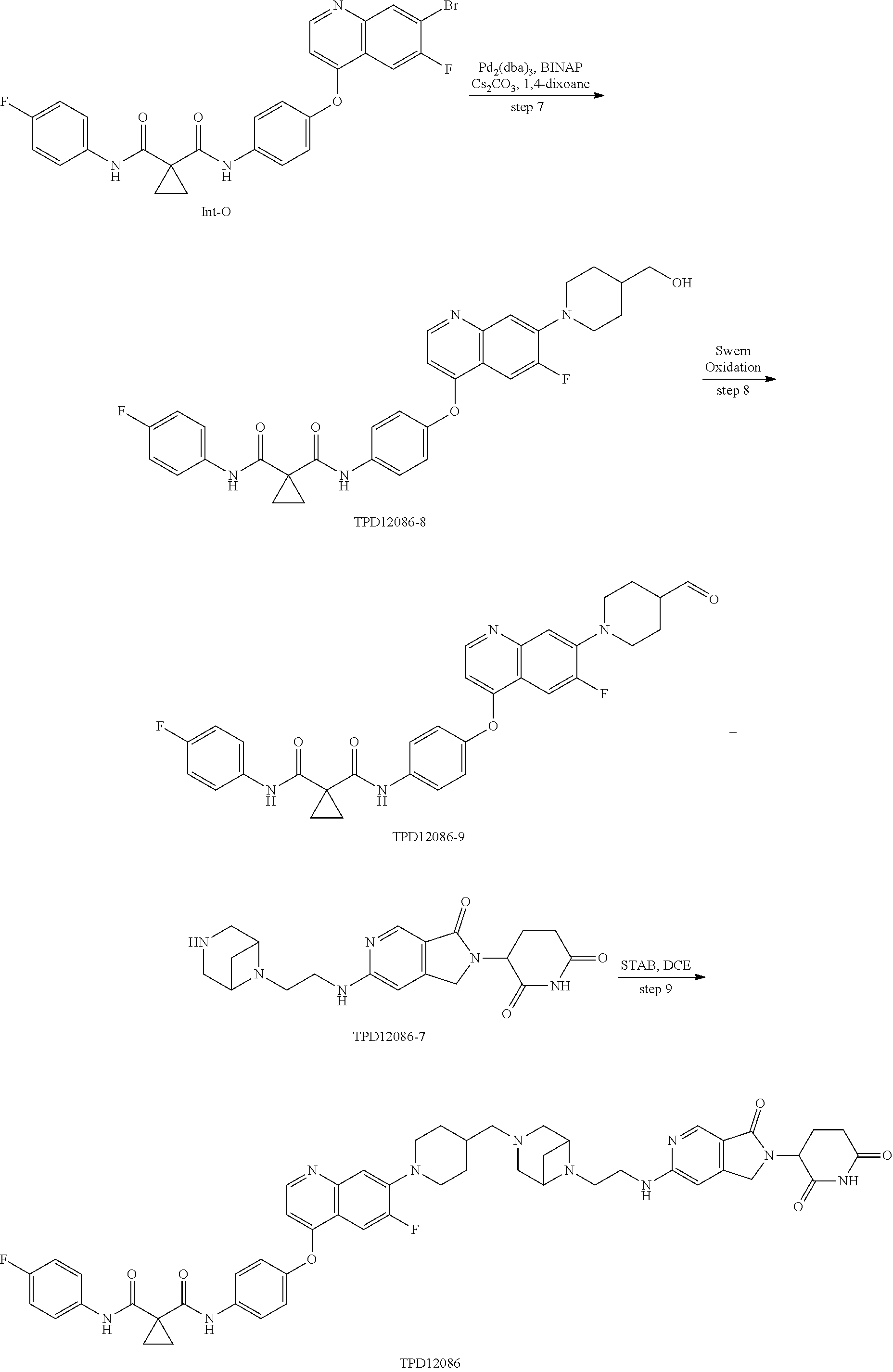 [SMILES: O=C(Nc1ccc(F)cc1)C1(C(=O)Nc2ccc(Oc3ccnc4cc(Br)c(F)cc34)cc2)CC1 ; O=C(Nc1ccc(F)cc1)C1(C(=O)Nc2ccc(Oc3ccnc4cc(N5CCC(CO)CC5)c(F)cc34)cc2)CC1 ; O=CC1CCN(c2cc3nccc(Oc4ccc(NC(=O)C5(C(=O)Nc6ccc(F)cc6)CC5)cc4)c3cc2F)CC1 ; O=C1CCC(N2Cc3cc(NCCN4C5CNCC4C5)ncc3C2=O)C(=O)N1 ; O=C1CCC(N2Cc3cc(NCCN4C5CC4CN(CC4CCN(c6cc7nccc(Oc8ccc(NC(=O)C9(C(=O)Nc%10ccc(F)cc%10)CC9)cc8)c7cc6F)CC4)C5)ncc3C2=O)C(=O)N1]

To a 500 ml triple-necked vial were sequentially added compound int-O-1 (10 g, 52.6 mmol), EtOH (200 ml) and 5-(methoxymethylene)-2,2-dimethyl-1,3-dioxahexane-4,6-dione (10.77 g, 57.8 mmol). The reaction was refluxed under nitrogen protection for 16 hours. After completion of the reaction, the reaction solution was reduced to room temperature and filtered by extraction to afford the compound int-O-2 (16 g, yellow solid, purity 99.855%), yield: 92.02%. LCMS (ESI) m/z calcd. for C₁₃H₁₁BrFNO₄ [M+H]⁺ 344.0; found 286.0; ¹H NMR (400 MHz, DMSO_d₆). 6=11.23 (d, J=14.0 Hz, 1H), 8.52 (d, J=14.3 Hz, 1H), 8.06 (dd, J=6.0, 2.8 Hz, 1H), 7.67-7.63 (m, 1H), 7.44 (t, J=8.7 Hz, 1H), 1.68 (s, 6H). The compound structure is confirmed.

Compound int-O-2 (8 g, 23.2 mmol) and diphenyl ether (80 ml) were added sequentially in a 250 ml single-necked vial. The reaction was carried out at 240° C. for 15 min under nitrogen protection. The other batch was operated identically. After completion of the reaction, the reaction solution was reduced to room temperature and the compound int-O-3 (7.8 g, brown solid, purity 52.957%) was obtained by filtration, yield: 36.13%. LCMS (ESI) m/z calcd. for C₉H₅BrFNO [M+H]⁺ 241.9; found 242.0, 244.0; ¹H NMR (400 MHz. DMSO_d₆): δ=11.90 (s, 1H), 7.99-7.91 (m, 1H), 7.86 (dd, J=11.3, 6.3 Hz, 1H), 7.67-7.57 (m, 1H), 6.05 (dd, J=13.9, 7.4 Hz, 1H). The compound structure is confirmed.

To a 250 ml triple-necked flask was added sequentially compound int-O-3 (7.8 g, 32.2 mmol), toluene (100 ml) and POCl₃ (14.81 g, 96.5 mmol). The reaction was carried out under nitrogen protection at 100° C. for 16 hours. After completion of the reaction, the reaction solution was reduced to room temperature, concentrated and adjusted to alkaline with saturated NaHCO₃ solution and extracted three times with DCM (100 ml). The organic phases were combined, washed with saturated brine, dried over anhydrous sodium sulfate, filtered, and concentrated to dryness, and the residue was purified by silica gel chromatographic column with eluent ratio of PE/EtOAc=50/1˜30/1 to obtain the compound int-O-4 (2.5 g, yellow solid, purity 98.538%) in the yield: 29.50%. LCMS (ESI) m/z calcd. for C9H₄BrClFN [M+H]⁺ 259.9; found 259.9, 262.0. The compound structure is confirmed.

Compound int-A-1 (20 g, 89.6 mmol), 4-aminophenol (13.69 g, 125.4 mmol), HATU (47.7 g, 125.4 mmol), DIEA (28.95 g, 224 mmol), and DMF (200 ml) were added sequentially to a 500 ml single-necked vial. The reaction was carried out under nitrogen protection at 25° C. for 16 hours. After the reaction was completed, the reaction solution was concentrated to dryness. The residue was purified by silica gel column with eluent ratio of DCM/MeOH=100/1˜30/1 to afford the compound int-A-2 (24 g, brown oil, purity 82.759%), yield: 70.54%. LCMS (ESI) m/z calcd. for C₁₇H₁₅FN₂O₃ [M+H]⁺ 315.1; found 315.1; ¹H NMR (400 MHz, DMSO_d₆): δ=10.17 (s, 1H), 9.74 (s, 1H), 9.23 (s, 1H), 7.64-7.61 (m, 2H), 7.35 (d, J=8.8 Hz, 2H), 7.16-7.12 (m, 2H), 6.70 (dd, J=7.0, 5.1 Hz, 2H). 1.44 (d, J=1.6 Hz, 4H). The compound structure is confirmed.

The compounds int-O-4 (2 g, 7.7 mmol), int-A-2 (2.90 g, 9.24 mmol), cesium carbonate (5.02 g, 15.4 mmol) and DMSO (30 ml) were added sequentially in a 100 ml triple vial. The reaction was carried out under nitrogen protection at 120° C. for 16 hours. After the reaction was completed, the reaction solution was reduced to room temperature, decanted into water (100 ml) and extracted three times with EA (100 ml). The organic phases were combined, washed with saturated brine, dried over anhydrous sodium sulfate, filtered, and concentrated to dryness. The residue was purified by silica gel column with eluent ratio of PE/EtOAc=5/1˜2/1 to give compound int-O (1.0 g, brown oil, purity 96.582%), yield: 23.38%. LCMS (ESI) m/z calcd. for C₂₆H₁₈BrF₂N₃O₃ [M+H]⁺ 538.1; found 538.1. 540.1; ¹H NMR (400 MHz, DMSO_d₆): δ=10.22 (s, 1H), 10.05 (s, 1H), 8.70 (d, J=5.1 Hz, 1H), 8.45 (d, J=6.7 Hz, 1H), 8.17 (d, J=9.4 Hz, 1H), 7.79 (d, J=8.9 Hz, 2H). 7.64 (dd, J=9.0, 5.1 Hz, 2H), 7.27 (d, J=9.0 Hz, 2H), 7.16 (t, J=8.9 Hz, 2H), 6.65 (d, J=5.0 Hz, 1H), 1.47 (s, 4H). The compound structure is confirmed.

Compound TPD12086-1 (5 g, 25.1 mmol), sodium bicarbonate (4.22 g, 50.2 mmol) and DCM/H2O=1/1 (100 ml) were added sequentially in a 250 ml triple-necked vial. CbzCl (5.14 g, 30.1 mmol) was added after the system was lowered to 0° C. The reaction was carried out under nitrogen protection at 25° C. for 16 hours. After completion of the reaction, the reaction solution was poured into water (100 ml) and extracted three times with DCM (30 ml). The organic phases were combined, washed with saturated brine, dried over anhydrous sodium sulfate, filtered, and concentrated to dryness, and the residue was purified by silica gel column with eluent ratio of PE/EtOAc=10/1˜5/1 to afford the compound TPD12086-2 (5.7 g, yellow oil, purity 96%) in the yield: 65.74%. LCMS (ESI) m/z calcd. for C₁₈H₂₄N₂O₄ [M+H]⁺ 333.2; found 233.2, 355.2; ¹H NMR (400 MHz, CDCl₃): δ=7.37-7.32 (m, 5H), 5.17 (s, 2H), 4.13 (d, J=21.6 Hz, 3H), 3.99 (s, 1H), 3.44 (d, J=12.2 Hz, 2H). 2.58 (dd, J=15.0, 6.5 Hz, 1H), 1.61 (s, 1H), 1.41 (s, 9H). The compound structure is confirmed.

To a 100 ml single-necked vial was sequentially added compound TPD12086-2 (5.7 g, 17.1 mmol), DCM (30 ml) and trifluoroacetic acid (60 ml). The reaction was carried out under nitrogen protection at 25° C. for 2 hours. After completion of the reaction it was concentrated to dryness to give the crude compound TPD12086-3 (6 g, brown oil), yield: 87.13%. LCMS (ESI) m/z calcd. for C₁₃H₁₆N₂O₂ [M+H]⁺ 233.1; found 233.1. The compound structure is confirmed.

Compound TPD12086-3 (6 g, 25.8 mmol), bromoacetonitrile (3.71 g, 31.0 mmol), potassium carbonate (10.70 g, 77.4 mmol) and ACN (100 ml) were added sequentially in a 250 ml single-necked vial. The reaction was carried out under nitrogen protection at 25° C. for 3 hours. After the reaction was completed, the reaction solution was poured into water (300 ml) and extracted three times with EA (100 ml). The organic phases were combined, washed with saturated brine, dried over anhydrous sodium sulfate, filtered, and concentrated to dryness, and the residue was purified by silica gel chromatographic column with eluent ratio of DCM/MeOH=200/1˜50/1 to afford the compound TPD12086-4 (5.4 g, brown oil, purity 92.063%) in the yield: 70.93%. LCMS (ESI) m/z calcd. for C₁₅H₁₇N₃O₂ [M+H]⁺ 272.1; found 272.2. The compound structure is confirmed.

To a 100 ml single-necked vial were sequentially added the compounds TPD12086-4 (4.4 g, 16.2 mmol), EtOH/NH H₃₂ O=10/1 (60 ml) and Raney nickel (2.85 g). The reaction was carried out under hydrogen (1 atm) atmosphere at 40° C. for 16 hours. After completion of the reaction, the reaction solution was filtered and concentrated to dryness, and the residue was purified by silica gel column with eluent ratio of DCM/MeOH=50/1˜30/1 to afford the compound TPD12086-5 (2 g, brown oil, purity 95.061%), yield: 42.59%. LCMS (ESI) m/z calcd. for C₁₅H₂₁N₃O₂ [M+H]⁺ 276.2; found 276.2; ¹H NMR (400 MHz, DMSO_d₆): δ=7.39-7.34 (m, 5H), 5.11 (s, 2H), 3.62 (d, J=12.1 Hz, 1H), 3.51 (t, J=15.4 Hz, 4H), 3.26 (d, J=11.4 Hz, 3H), 2.40-2.31 (m, 4H), 1.41 (d, J=8.4 Hz, 1H). The compound structure is confirmed.

To a 25 ml triple-necked vial were sequentially added compound TPD12086-5 (500 mg, 1.82 mmol), compound TPD12003-4 (508 mg, 1.82 mmol), DIEA (469 mg, 3.63 mmol) and DMSO (5 ml). The reaction was carried out under nitrogen protection at 120° C. for 16 hours. After completion of the reaction, the reaction solution was reduced to room temperature, decanted into water (30 ml) and extracted three times with EA (30 ml). The organic phases were combined, washed with saturated brine, dried over anhydrous sodium sulfate, filtered, and concentrated to dryness. The residue was purified by silica gel column with eluent ratio of DCM/MeOH=50/1˜30/1 to afford the compound TPD12086-6 (200 mg, brown solid, purity 86.411%), yield: 21.24%. LCMS (ESI) m/z calcd. for C₂₇H₃₀N₆O₅ [M+H]⁺ 519.2; found 519.1. The compound structure is confirmed.

Compound TPD12086-6 (80 mg, 0.15 mmol) and trifluoroacetic acid (2 ml) were added sequentially in a 25 ml single-necked vial. The reaction was carried out under nitrogen protection at 75° C. for 2 hours. After completion of the reaction it was concentrated to dryness to give the crude compound TPD12086-7 (100 mg, brown solid), yield: 54.50%. LCMS (ESI) m/z calcd. for C₁₉H₂₄N₆O₃ [M+H]⁺ 385.2; found 385.2. The compound structure is confirmed.

A 25 ml single-necked vial was sequentially charged with compound int-O (250 mg, 0.46 mmol), 4-hydroxymethylpiperidine (107 mg, 0.93 mmol), Pd₂ (dba)₃ (43 mg, 0.046 mmol), BINAP (58 mg, 0.093 mmol), cesium carbonate (303 mg, 0.93 mmol), and 1,4-dioxane (10 ml). The reaction was carried out for 16 h at 100° C. under nitrogen protection. The other five batches were operated identically. After the reaction was completed, the reaction solution was brought to room temperature, decanted into water (1200 ml) and extracted three times with EA (50 ml). The organic phases were combined, washed with saturated brine, dried over anhydrous sodium sulfate, filtered, and concentrated to dryness. The residue was purified by silica gel column with eluent ratio of DCM/MeOH=50/1˜30/1 to afford the compound TPD12086-8 (310 mg, yellow solid, purity 91.691%), yield: 17.82%. LCMS (ESI) m/z calcd. for C₃₂H₃₀F₂N₄O₄ [M+H]⁺ 573.2; found 573.2; ¹H NMR (400 MHz, DMSO_d₆): δ=10.20 (s, 1H), 10.06 (s, 1H), 8.56 (d, J=5.2 Hz, 1H), 7.86 (d, J=13.5 Hz, 1H), 7.76 (d, J=9.0 Hz, 2H), 7.66-7.63 (m, 2H), 7.45 (d, J=8.5 Hz, 1H), 7.24-7.20 (m, 2H), 7.18-7.13 (m, 2H), 6.45 (d, J=5.2 Hz, 1H), 4.54 (t, J=5.3 Hz, 1H), 3.58 (d, J=11.4 Hz, 2H), 3.33 (s, 1H), 2.77 (t, J=11.1 Hz, 2H), 1.82 (d, J=10.8 Hz, 2H), 1.58 (br. s, 1H), 1.47 (s, 4H), 1.38-1.23 (m, 3H). The compound structure is confirmed.

Compound oxalyl chloride (137 mg, 1.08 mmol) and DCM (4 ml) were sequentially added to a 50 ml triple-necked vial, cooled down to −78° C. A DCM (2 ml) solution of DMSO (169 mg, 2.17 mmol) was added and −78° C. stirred for 0.5 hours. Then DCM (4 ml) solution of compound TPD12086-8 (310 mg, 0.54 mmol) was added and −78° C. stirred for 0.5 hours. Finally, TEA (438 mg, 4.33 mmol) was added and the reaction was slowly returned to room temperature and protected by nitrogen for 1 hour. After completion of the reaction, the reaction solution was poured into saturated NaHCO₃ solution (30 ml) and extracted three times with DCM (30 ml). The organic phases were combined, washed with saturated brine, dried over anhydrous sodium sulfate, filtered, and concentrated to dryness, and the residue was purified by preparative plate with the unfolding agent ratio of DCM/MeOH=12/1 to afford the compound TPD12086-9 (180 mg, yellow solid, purity 95.968%), yield: 55.91%. LCMS (ESI) m/z calcd. for C₃₂H₂₈F₂N₄O₄ [M+H]⁺ 571.2; found 571.3; ¹H NMR (400 MHz, DMSO_d₆): δ=10.19 (s, 1H), 10.06 (s, 1H), 9.68 (s, 1H), 8.57 (d, J=5.2 Hz, 1H), 7.87 (d, J=13.4 Hz, 1H), 7.76 (d, J=8.9 Hz, 2H), 7.64 (dd, J=9.0, 5.1 Hz, 2H), 7.46 (d, J=8.5 Hz, 1H), 7.22 (d, J=8.9 Hz, 2H), 7.15 (t, J=8.9 Hz, 2H), 6.46 (d, J=5.2 Hz, 1H), 3.52-3.49 (m, 2H). 2.94 (t, J=10.3 Hz, 2H), 2.73 (s, 1H), 2.04 (d, J=10.3 Hz, 2H), 1.77-1.68 (m, 2H), 1.47 (s, 4H). The compound structure is confirmed.

To a 25 ml single-necked vial was sequentially added compound TPD12086-7 (50 mg, 0.13 mmol), compound TPD12086-9 (74 mg, 0.13 mmol), sodium triacetoxyborohydride (55 mg, 0.26 mmol) and DCE (3 ml). The reaction was carried out under nitrogen protection at 25° C. for 16 hours. After the reaction was completed, the reaction solution was decanted into water (15 ml) and an appropriate amount of saturated NaHCO₃ solution was added to make the reaction solution alkaline. The organic phase was separated and the aqueous phase was extracted twice with DCM (10 ml). The organic phases were combined, washed with saturated brine, dried over anhydrous sodium sulfate, filtered, and concentrated to dryness. The residue was prepared by high performance liquid chromatography (HPLC) with the following parameters: column: sunfire 5 μm 19-150 mm; mobile phase: acetonitrile-water (0.1% FA); gradient: 20-35/7 min to afford the compound TPD12086 (13.26 mg, yellow solid, purity 98.425%) in the following yield: 10.68%. LCMS (ESI) m/z calcd. for C₅₁H₅₂F₂N₁₀O₆ [M+H]⁺ 939.4; found 470.2; 939.1; ¹H NMR (400 MHz, DMSO_d₆): δ=10.98 (s, 1H), 10.20 (s, 1H), 10.06 (s, 1H), 8.56 (d, J=5.2 Hz, 1H), 8.38 (s. 1H), 8.22 (s, 2H), 7.86 (d, J=13.4 Hz, 1H), 7.76 (d, J=8.8 Hz, 2H), 7.64 (dd, J=8.9, 5.1 Hz, 2H), 7.44 (d, J=8.5 Hz, 1H), 7.23-7.13 (m, 5H), 6.59 (s, 1H), 6.45 (d J=5.2 Hz, 1H), 5.04 (dd, J=13.2, 5.0 Hz, 1H), 4.33 (d, J=17.4 Hz, 1H), 4.19 (d, J=17.5 Hz, 1H), 3.71 (s, 3H), 2.98-2.84 (m, 6H), 2.79-2.74 (m, 4H), 2.41-2.22 (m, 4H), 1.93-1.83 (m, 5H), 1.73 (s, 1H), 1.47 (s, 4H), 1.33-1.23 (m, 4H). The compound structure is confirmed.

### Example 67: Synthesis of Compound TPD12088

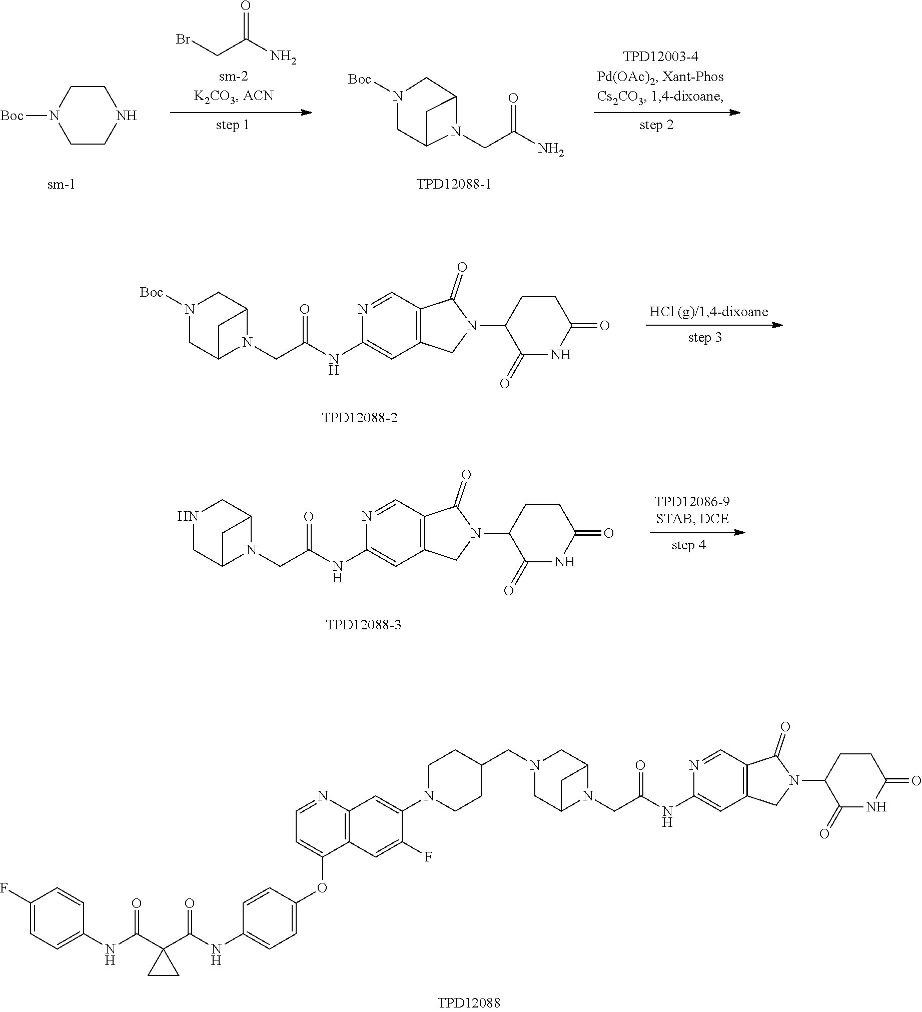 [SMILES: NC(=O)CBr ; CC(C)(C)OC(=O)N1CCNCC1 ; CC(C)(C)OC(=O)N1CC2CC(C1)N2CC(N)=O ; CC(C)(C)OC(=O)N1CC2CC(C1)N2CC(=O)Nc1cc2c(cn1)C(=O)N(C1CCC(=O)NC1=O)C2 ; O=C1CCC(N2Cc3cc(NC(=O)CN4C5CNCC4C5)ncc3C2=O)C(=O)N1 ; O=C1CCC(N2Cc3cc(NC(=O)CN4C5CC4CN(CC4CCN(c6cc7nccc(Oc8ccc(NC(=O)C9(C(=O)Nc%10ccc(F)cc%10)CC9)cc8)c7cc6F)CC4)C5)ncc3C2=O)C(=O)N1]

To a 50 ml single-necked vial was sequentially added compound sm-1 (500 mg, 2.51 mmol), 2-bromoacetamide (415 mg, 3.01 mmol), potassium carbonate (1040 mg, 7.53 mmol) and acetonitrile (10 ml). The reaction was carried out under nitrogen protection at 25° C. for 2 hours. After the reaction was completed, the reaction solution was poured into water (30 ml) and extracted three times with EA (10 ml). The organic phases were combined, washed with saturated brine, dried over anhydrous sodium sulfate, filtered, and concentrated to dryness, and the residue was purified by silica gel chromatographic column with eluent ratio of DCM/MeOH=100/1˜30/1 to afford the compound TPD12088-1 (410 mg, white solid, purity 92.085%) in the yield: 58.70%. LCMS (ESI) m/z calcd. for C₁₂H₂₁N₃O₃ [M+H]⁺ 256.2; found 255.8; ¹H NMR (400 MHz, DMSO_d₆): δ=7.23 (s, 1H), 7.09 (s, 1H), 3.56-3.39 (m, 4H), 3.28-3.20 (m, 2H), 2.86 (s, 2H), 2.43 (dd. J=13.9, 6.4 Hz, 1H), 1.43 (s, 9H), 1.38 (d, J=8.5 Hz, 1H). The compound structure is confirmed.

To a 25 ml single-necked vial was sequentially added compound TPD12088-1 (410 mg, 1.60 mmol), compound TPD12003-4 (537 mg, 1.92 mmol), palladium acetate (72 mg, 0.32 mmol), Xant-Phos (185 mg, 0.32 mmol), cesium carbonate (782 mg. 2.40 mmol) and 1,4-dioxane (10 ml). The reaction was carried out under nitrogen protection at 100° C. for 16 hours. After completion of the reaction, the reaction solution was brought to room temperature, decanted into water (30 ml) and extracted three times with EA (10 ml). The organic phases were combined, washed with saturated brine, dried over anhydrous sodium sulfate, filtered, and concentrated to dryness. The residue was purified by silica gel column with eluent ratio of DCM/MeOH=100/1˜30/1 to afford the compound TPD12088-2 (200 mg, yellow solid, purity 93.691%), yield: 23.45%. LCMS (ESI) m/z calcd. for C₂₄H₃₀N₆O₆ [M+H]⁺ 499.2; found 499.1; ¹H NMR (400 MHz, DMSO_d₆): δ=11.03 (s, 1H), 10.44 (s, 1H), 8.70 (s, 1H), 8.32 (s, 1H), 5.10 (dd, J=13.3, 5.1 Hz, 1H), 4.55 (d, J=18.5 Hz, 1H), 4.40 (d, J=18.5 Hz, 1H), 3.68-3.49 (m, 4H), 3.34-3.26 (m, 5H), 2.95-2.86 (m, 1H), 2.63-2.54 (m, 2H), 2.43-2.34 (m, 1H), 2.02-1.99 (m, 1H), 1.44 (s, 9H). The compound structure is confirmed.

To a 25 ml single-necked vial was sequentially added compound TPD12088-2 (200 mg, 0.40 mmol), 1,4-dioxane (1 ml) and 4N HCl/1,4-dioxane (4 ml). The reaction was carried out under nitrogen protection at 25° C. for 2 hours. After completion of the reaction it was concentrated to dryness to give the crude compound TPD12088-3 (200 mg, off-white solid), yield: 102.57%. LCMS (ESI) m/z calcd. for C₁₉H₂₂N₆O₄ [M+H]⁺ 399.2; found 399.0. The compound structure is confirmed.

To a 25 ml single-necked vial was sequentially added compound TPD12088-3 (100 mg, 0.25 mmol), compound TPD12086-9 (143 mg, 0.25 mmol), sodium triacetoxyborohydride (106 mg, 0.50 mmol) and DCE (3 ml). The reaction was carried out under nitrogen protection at 25° C. for 16 hours. After the reaction was completed, the reaction solution was decanted into water (15 ml) and an appropriate amount of saturated NaHCO₃ solution was added to make the reaction solution alkaline. The organic phase was separated and the aqueous phase was extracted twice with DCM (5 ml). The organic phases were combined, washed with saturated brine, dried over anhydrous sodium sulfate, filtered, and concentrated to dryness. The residue was prepared by high performance liquid chromatography (HPLC) with the following parameters: column: sunfire 5 μm 19-150 mm; mobile phase: acetonitrile-water (0.1% FA); gradient: 20-35/7 min to afford the compound TPD12088 (19.5 mg, yellow solid, purity 98.134%) in the following yield: 7.73%. LCMS (ESI) m/z calcd. for C₅₁H₅₀F₂N₁₀O₇ [M+H]⁺ 953.4; found 953.2; ¹H NMR (400 MHz, DMSO_d₆): δ=11.02 (s, 1H), 10.41 (s, 1H), 10.20 (s, 1H), 10.06 (s, 1H), 8.71 (s, 1H), 8.56 (d, J=5.2 Hz, 1H), 8.33 (s, 1H), 8.19 (s, 0.7H), 7.86 (d, J=13.5 Hz, 1H), 7.76 (d, J=8.8 Hz, 2H), 7.64 (dd, J=8.9, 5.1 Hz, 2H), 7.45 (d, J=8.5 Hz, 1H), 7.22 (d, J=8.9 Hz, 2H), 7.15 (t, J=8.9 Hz, 2H), 6.45 (d, J=5.2 Hz, 1H), 5.11 (dd, J=13.3, 5.1 Hz, 1H), 4.55 (d, J=18.5 Hz, 1H), 4.41 (d, J=18.5 Hz, 1H), 3.64-3.58 (m, 5H), 2.95-2.78 (m, 8H), 2.67-2.53 (m, 3H), 2.43-2.33 (m, 2H), 2.01-1.99 (m, 1H), 1.87 (dd, J=20.3, 10.1 Hz, 3H), 1.78 (s, 1H), 1.47 (s, 4H), 1.34-1.32 (m, 2H). The compound structure is confirmed.

### Example 68: Synthesis of Compound TPD12096

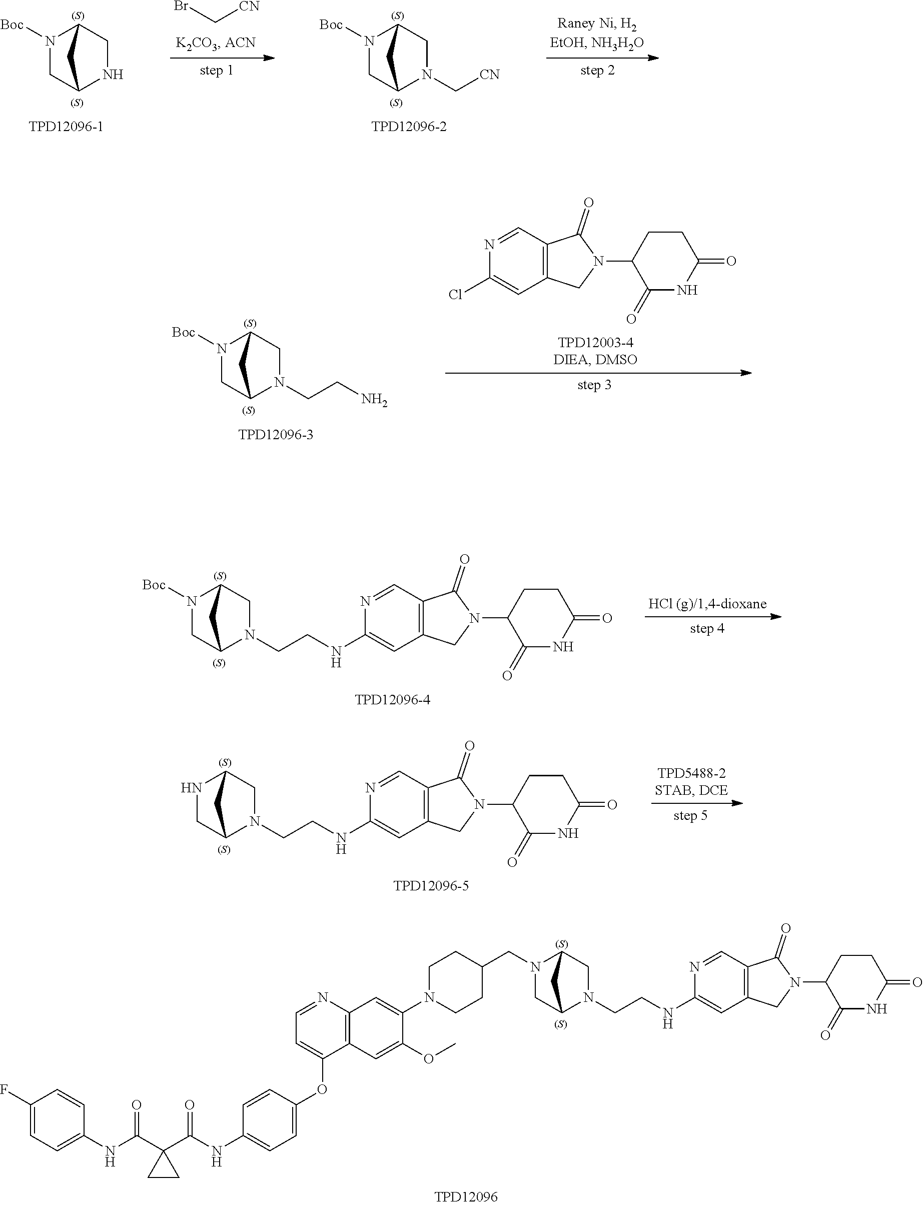

To a 100 ml single-necked vial were sequentially added compound TPD12096-1 (2 g, 0.01 mol), bromoacetonitrile (1.44 g, 0.012 mol), K₂CO₃ (4.15 g, 0.03 mol), and ACN (30 ml). The reaction was carried out under nitrogen protection at 25° C. for 16 hours. After the reaction was completed, the reaction solution was brought to room temperature, decanted into water (90 ml) and extracted three times with EA (30 ml). The organic phases were combined, washed with saturated brine, dried over anhydrous sodium sulfate, filtered, and concentrated to dryness to afford the compound TPD12096-2 (2 g, yellow oil, purity 82.6%), yield: 69.00%. LCMS (ESI) m/z calcd. for C₁₂H₁₉N₃O₂ [M+H]⁺ 238.2; found 238.2; ¹H NMR (400 MHz, DMSO_d MHz, DMSO_d₆): δ=4.20 (d, J=9.6 Hz, 1H), 3.82-3.66 (m, 2H), 3.32-3.17 (m, 2H), 3.10 (t, J=10.0 Hz, 1H), 2.81-2.65 (m, 2H), 1.80 (d, J=9.5 Hz, 1H), 1.63 (t, J=9.8 Hz, 1H), 1.39 (d, J=3.5 Hz, 9H). The compound structure is confirmed.

To a 100 ml single-necked vial were sequentially added compounds TPD12096-2 (1 g, 4.20 mmol), Raney Ni (0.74 g, 12.6 mol), EtOH (20 ml) and NH H₃₂ O (2 ml). The reaction was carried out for 8 hours at 40° C. under hydrogen protection. After completion of the reaction, the reaction solution was reduced to room temperature, filtered and concentrated to dryness. The residue was purified by silica gel column with eluent ratio of DCM/MeOH=50/1˜30/1 to afford the compound TPD12096-3 (0.7 g, yellow oil), yield: 54.76%. LCMS (ESI) m/z calcd. for C₁₂H₂₃N₃O₂ [M+H]⁺ 242.2; found 242.2; ¹H NMR (400 MHz, DMSO_d MHz, DMSO_d₆): δ=4.11 (d, J=9.7 Hz, 1H), 3.04 (dd, J=13.8, 10.1 Hz, 2H), 2.79 (d, J=9.2 Hz, 1H), 2.55-2.52 (m, 1H), 2.50-2.43 (m, 4H), 2.39 (d, J=9.5 Hz, 1H). 1.69 (d, J=9.4 Hz, 1H), 1.58 (dd, J=20.3, 10.2 Hz, 1H), 1.39 (s, 9H). The compound structure is confirmed.

To a 25 ml single-necked vial were sequentially added compound TPD12096-3 (286 mg, 1.18 mmol), compound TPD12003-4 (300 mg, 1.08 mmol), DIEA (208 mg, 1.61 mmol) and DMSO (4 ml). The reaction was carried out under nitrogen protection at 130° C. for 16 hours. After completion of the reaction, the reaction solution was reduced to room temperature, decanted into water (20 ml) and extracted three times with EA (15 ml). The organic phases were combined, washed with saturated brine, dried over anhydrous sodium sulfate, filtered, and concentrated to dryness. The residue was purified by preparative plate with the unfolding agent ratio of DCM/MeOH=10/1 to afford the compound TPD12096-4 (130 mg, yellow solid, purity 72.993%), yield: 18.22%. LCMS (ESI) m/z calcd. for C₂₄H₃₂N₆O₅ [M+H]⁺ 485.2; found 485.2; ¹H NMR (400 MHz, DMSO_d₆): δ=10.96 (s, 1H), 8.34 (s, 1H), 7.13 (s, 1H), 6.58 (s, 1H), 5.76 (s, 1H), 5.02 (dd, J=13.3, 5.0 Hz, 1H), 4.32 (d, J=17.4 Hz, 1H), 4.21-4.11 (m 2H), 3.50 (d, J=8.9 Hz, 1H), 3.17 (d, J=5.1 Hz, 1H), 3.10-3.01 (m, 2H), 2.92-2.83 (m, 2H), 2.59 (ddd, J=32.9, 15.3 Hz, 4H), 2.32 (ddd, J=26.4 13.3, 4.3 Hz, 2H), 1.98-1.90 (m, 1H), 1.67 (dd, J=30.9, 10.4 Hz, 2H), 1.38 (s, 10H). The compound structure is confirmed.

To a 25 ml single-necked vial were sequentially added compound TPD12096-4 (130 mg, 0.27 mmol) and 4N HCl/1,4-dioxane (5 ml). The reaction was carried out at 25° C. for 2 h. After completion of the reaction, it was concentrated to dryness to give the crude compound TPD12096-5 (150 mg, yellow solid), yield: 76.84%. LCMS (ESI) m/z calcd. for C₁₉H₂₄N₆O₃ [M+H]⁺ 385.2; found 385.2. The compound structure is confirmed.

To a 25 ml single-necked vial was sequentially added compound TPD12096-5 (150 mg, 0.39 mol), compound TPD5488-2 (273 mg, 0.47 mmol), sodium triacetoxyborohydride (165 mg, 0.78 mmol) and DCE (3 ml). The reaction was carried out under nitrogen protection at 25° C. for 16 hours. After the reaction was completed, the reaction solution was poured into water (15 ml) and an appropriate amount of saturated NaHCO₃ solution was added to make the reaction solution alkaline. It was extracted three times with DCM (10 ml). The organic phases were combined, washed with saturated brine, dried over anhydrous sodium sulfate, filtered, and concentrated to dryness. The residue was prepared by high performance liquid chromatography (HPLC) with the following parameters: column: XBridge-1 5 μm 19-150 mm; mobile phase: acetonitrile-water (0.1% FA); gradient: 10-55/8 min to afford the compound TPD12096 (54.6 mg, yellow solid, purity 95.636%) in the following yield: 14.07%. LCMS (ESI) m/z calcd. for C₅₂H₅₅FN₁₀O₇ [M+H]⁺ 951.4; found 951.4; ¹H NMR (400 MHz, DMSO_d₆): δ=10.99 (s, 1H), 10.19 (s, 1H), 10.07 (s, 1H), 8.43 (d, J=5.2 Hz, 1H), 8.37 (s, 1H). 8.22 (s, 2H), 7.76 (d, J=8.9 Hz, 2H), 7.64 (dd, J=9.0, 5.1 Hz, 2H), 7.46 (s, 1H), 7.32 (s, 1H), 7.23-7.12 (m, 5H), 6.60 (s, 1H), 6.39 (d, J=5.2 Hz, 1H), 5.03 (dd. J=13.3, 5.1 Hz, 1H), 4.33 (d, J=17.4 Hz, 1H), 4.19 (d, J=17.4 Hz, 1H), 3.94 (s, 3H), 3.59 (d, J=10.7 Hz, 2H), 3.48 (s, 1H), 3.41 (s, 3H), 2.83-2.57 (m, 9H), 2.39-2.26 (m, 2H), 1.98-1.90 (m, 1H), 1.85 (d, J=11.3 Hz, 2H), 1.69 (s, 2H), 1.56 (s, 1H), 1.47 (s, 4H), 1.35 (s, 2H). The compound structure is confirmed.

### Example 69: Synthesis of Compound TPD12097

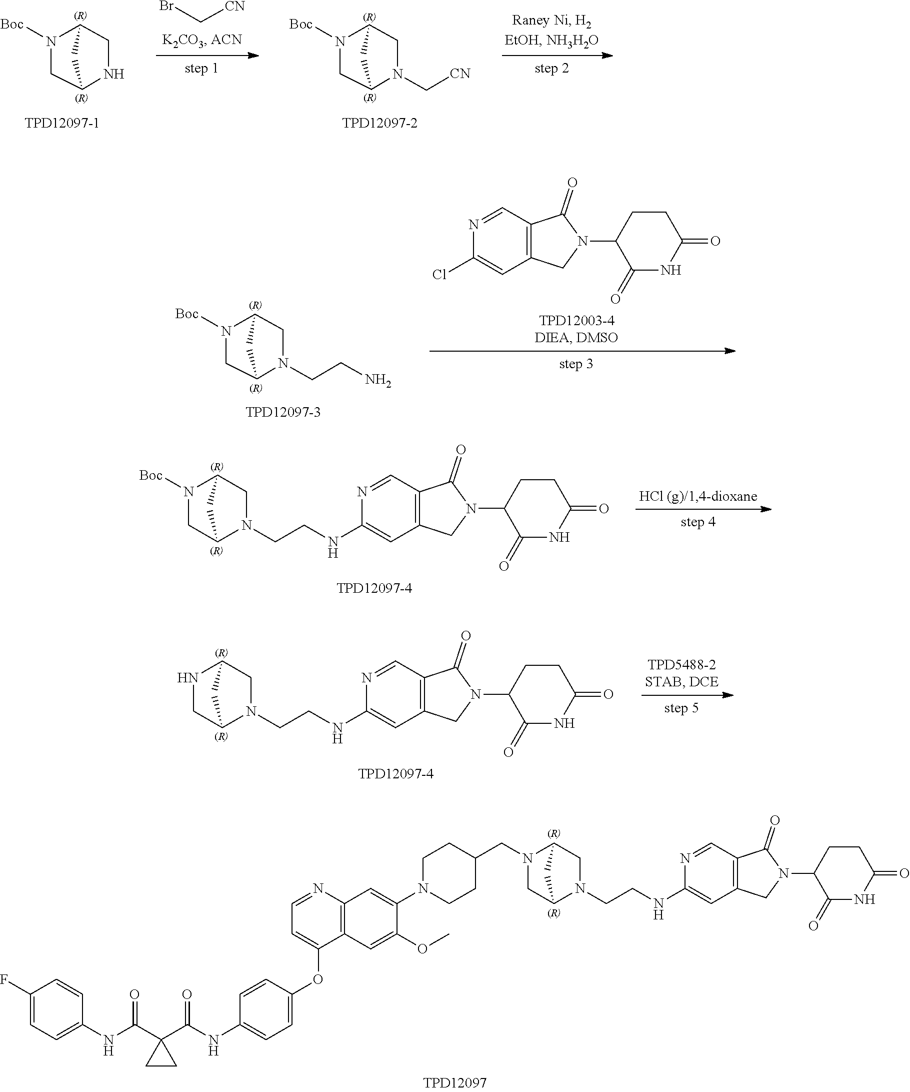

To a 100 ml single-necked vial were sequentially added compound TPD12097-1 (2 g, 0.01 mol), bromoacetonitrile (1.44 g, 0.012 mol), K₂CO₃ (4.15 g, 0.03 mol), and ACN (30 ml). The reaction was carried out under nitrogen protection at 25° C. for 16 hours. After the reaction was completed, the reaction solution was brought to room temperature, decanted into water (90 ml) and extracted three times with EA (60 ml). The organic phases were combined, washed with saturated brine, dried over anhydrous sodium sulfate, filtered, and concentrated to dryness to afford the compound TPD12097-2 (2 g, yellow oil, purity 81.383%), yield: 68%. LCMS (ESI) m/z calcd. for C₁₂H₁₉N₃O₂ [M+H]⁺ 238.2; found 237.9; ¹H NMR (400 MHz, DMSO_d₆): δ=4.20 (d, J=9.6 Hz, 1H), 3.81-3.66 (m, 2H), 3.55 (s, 1H), 3.31 (d, J=10.5 Hz, 1H), 3.10 (t, J=10.2 Hz, 1H), 2.79 (d, J=8.4 Hz, 1H), 2.69 (d, J=9.4 Hz, 1H), 1.80 (d, J=8.8 Hz, 1H), 1.63 (t, J=9.9 Hz, 1H), 1.39 (d, J=3.2 Hz, 9H). The compound structure is confirmed.

To a 100 ml single-necked vial were sequentially added compounds TPD12097-2 (2 g, 8.43 mol), Raney Ni (0.59 g), EtOH (24 ml) and NH H₂O (3 ml). The reaction was carried out for 16 hours at 40° C. under hydrogen protection. After completion of the reaction, the reaction solution was reduced to room temperature, filtered and concentrated to dryness. The residue was purified by silica gel column with eluent ratio of DCM/MeOH=120/1˜30/1 to afford compound TPD12097-3 (1.8 g, yellow oil, purity 56.329%), yield: 50.00%. LCMS (ESI) m/z calcd. for C₁₂H₂₃N₃O₂ [M+H]⁺ 242.2; found 242.1; ¹H NMR (400 MHz, DMSO_d₆): δ=4.12 (d, J=10.7 Hz, 1H), 3.40 (s, 1H), 3.30 (dd, J=9.0, 6.3 Hz, 1H), 3.09-3.02 (m, 1H), 2.79 (d, J=9.3 Hz, 1H), 2.54-2.38 (m, 6H). 1.69 (d, J=9.5 Hz, 1H), 1.63-1.56 (m, 1H), 1.39 (s, 10H). The compound structure is confirmed.

To a 25 ml single-necked vial were sequentially added compound TPD12097-3 (334 mg, 1.38 mmol), compound TPD12003-4 (350 mg, 1.25 mmol), DIEA (323 mg, 2.50 mmol) and DMSO (4 ml). The reaction was carried out under nitrogen protection at 130° C. for 16 hours. After completion of the reaction, the reaction solution was reduced to room temperature, decanted into water (20 ml) and extracted three times with EA (15 ml). The organic phases were combined, washed with saturated brine, dried over anhydrous sodium sulfate, filtered, and concentrated to dryness. The residue was purified by preparative plate with unfolding agent ratio DCM/MeOH=10/1 to give compound TPD12097-4 (52 mg, yellow solid, purity 42.921%), yield: 48.91%. LCMS (ESI) m/z calcd. for C₂₃H₃₂N₆O₅ [M+H]⁺ 485.2; found 485.1. The compound structure is confirmed.

Compound TPD12097-4 (150 mg, 0.31 mmol) 4N HCl/1,4-dioxane (4 ml) was added sequentially in a 25 ml single-necked vial. The reaction was carried out at 25° C. for 2 h. After completion of the reaction, it was concentrated to dryness to give the crude compound TPD12097-5 (280 mg, yellow solid), yield: 119.52%. LCMS (ESI) m/z calcd. for C₁₈H₂₄N₆O₃ [M+H]⁺ 385.2; found 385.2. The compound structure is confirmed.

To a 25 ml single-necked vial were sequentially added compound TPD12097-5 (280 mg, 0.73 mmol), compound TPD5488-2 (424 mg, 0.73 mmol), sodium triacetoxyborohydride (309 mg, 1.46 mmol) and DCE (8 ml). The reaction was carried out under nitrogen protection at 25° C. for 16 hours. After the reaction was completed, the reaction solution was poured into water (40 ml) and an appropriate amount of saturated NaHCO₃ solution was added to make the reaction solution alkaline. It was extracted three times with DCM (20 ml). The organic phases were combined, washed with saturated brine, dried over anhydrous sodium sulfate, filtered, and concentrated to dryness. The residue was prepared by high performance liquid chromatography (HPLC) with the following parameters: column: XBridge-1 5 μm 19-150 mm; mobile phase: acetonitrile-water (0.1% FA); gradient: 10-55/8 min to give compound TPD12097 (6.68 mg, yellow solid, purity 97.125%) in the following yield: 0.93%. LCMS (ESI) m/z calcd. for C₅₁H₅₅FN₁₀O₇ [M+H]⁺ 951.4; found 951.4; ¹H NMR (400 MHz, DMSO_d₆): δ=10.98 (s, 1H), 10.19 (s, 1H), 10.07 (s, 1H), 8.46-8.17 (m, 3H), 7.76 (d, J=7.7 Hz, 2H), 7.64 (s, 2H), 7.46 (s, 1H), 7.32 (s, 1H), 7.25-7.03 (m, 4H), 6.59 (s, 1H), 6.39 (s, 1H), 5.03 (d, J=8.5 Hz, 1H), 4.33 (d, J=17.6 Hz, 1H), 4.18 (d, J=17.1 Hz, 1H), 3.94 (s, 3H), 3.57 (s, 10H), 2.68 (d, J=12.0 Hz, 8H), 1.95 (s, 2H), 1.84 (s, 2H), 1.61 (s, 2H), 1.47 (s, 5H), 1.24 (s, 5H). The compound structure is confirmed.

### Example 70: Synthesis of Compound TPD12100

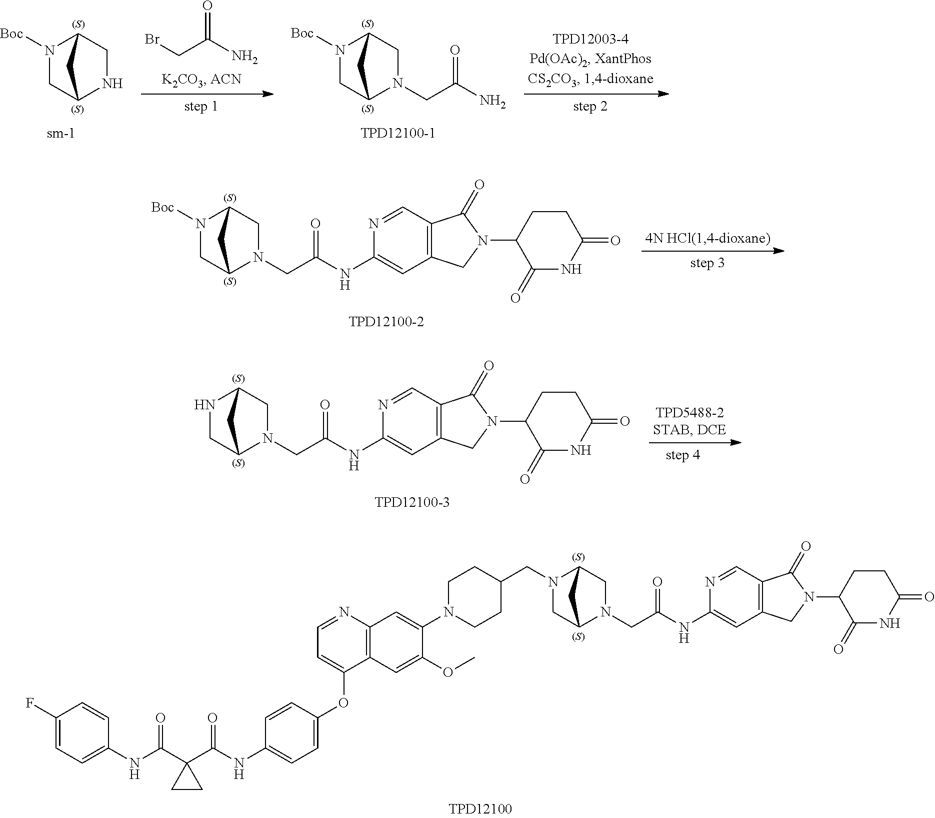

To a 100 ml single-necked vial were sequentially added compound sm-1 (1 g, 0.005 mol), 2-bromoacetamide (0.72 g, 0.0052 mol), K₂CO₃ (1.04 g, 0.0075 mol), and ACN (10 ml). The reaction was carried out under nitrogen protection at 25° C. for 16 hours. After the reaction was completed, the reaction solution was brought to room temperature, decanted into water (30 ml) and extracted three times with EA (15 ml). The organic phases were combined, washed with saturated brine, dried over anhydrous sodium sulfate, filtered, and concentrated to dryness. The residue was purified by silica gel column with eluent ratio of DCM/MeOH=50/1˜30/1 to obtain the compound TPD12100-1 (1.2 g, white solid, purity 67.57%), yield: 64.00%. LCMS (ESI) m/z calcd. for C₁₂H₂₁N₃O₃ [M+H]⁺ 256.16; found 256.1; ¹H NMR (400 MHz, DMSO_d₆): δ=7.22 (s, 1H), 7.10 (s, 1H), 4.13 (t, J=10.7 Hz, 1H), 3.81 (s, 1H), 3.43 (s, 1H), 3.31 (d, J=4.4 Hz, 1H), 3.04 (dd, J=31.6, 18.1 Hz, 3H), 2.83 (d, J=8.9 Hz, 1H), 1.75 (d, J=9.5 Hz, 1H), 1.63-1.53 (m, 1H), 1.39 (s, 9H). The compound structure is confirmed.

To a 25 ml single-necked vial was sequentially added compound TPD12100-1 (450 mg, 1.76 mmol), compound TPD12003-4 (737 mg, 2.63 mmol), XantPhos (203 mg, 0.35 mmol), Pd(OAc)₂ (79 mg, 0.35 mmol), CS₂CO₃ (1430 mg, 4.39 mmol) and 1,4-dioxane (5 ml). The reaction was carried out under nitrogen protection at 100° C. for 16 hours. After completion of the reaction, the reaction solution was brought to room temperature, decanted into water (30 ml) and extracted three times with DCM (20 ml). The organic phases were combined, washed with saturated brine, dried over anhydrous sodium sulfate, filtered, and concentrated to dryness. The residue was purified by preparative plate with the unfolding agent ratio of DCM/MeOH=12/1 to afford the compound TPD12100-2 (180 mg, yellow solid, purity 98.889%), yield: 20.30%. LCMS (ESI) m/z calcd. for C₂₄H₃₀N₆O₆ [M+H]⁺ 499.22; found 499.1; ¹H NMR (400 MHz, DMSO_d₆): δ=11.02 (s, 1H), 10.30 (s, 1H), 8.70 (s, 1H), 8.33 (s, 1H), 7.17 (d, J=47.5 Hz, 3H), 5.76 (s, 1H), 5.11 (dd, J=13.3, 5.0 Hz, 1H), 4.55 (d, J=18.5 Hz, 1H), 4.41 (d, J=18.6 Hz, 1H), 4.18 (dd, J=23.4, 12.7 Hz, 2H), 3.63-3.37 (m, 5H), 2.63 (d, J=9.3 Hz, 2H), 2.07-1.96 (m, 1H), 1.41 (s, 9H). The compound structure is confirmed.

To a 25 ml single-necked vial was sequentially added compound TPD12100-2 (180 mg, 0.36 mmol) and 4N HCl/1,4-dioxane (5 ml). The reaction was carried out at 25° C. for 2 h. After completion of the reaction, it was concentrated to dryness to give the crude compound TPD12100-3 (200 mg, yellow solid, purity 70.214%), yield: 97.84%. LCMS (ESI) m/z calcd. for C₁₉H₂₂N₆O₄ [M+H]⁺ 399.17; found 399.1. The compound structure is confirmed.

To a 25 ml single-necked vial was sequentially added compound TPD12100-3 (200 mg, 0.50 mmol), compound TPD5488-2 (273 mg, 0.47 mmol), sodium triacetoxyborohydride (213 mg, 1.00 mmol) and DCE (4 ml). The reaction was carried out under nitrogen protection at 25° C. for 16 hours. After the reaction was completed, the reaction solution was poured into water (15 ml) and an appropriate amount of saturated NaHCO₃ solution was added to make the reaction solution alkaline. It was extracted three times with DCM (10 ml). The organic phases were combined, washed with saturated brine, dried over anhydrous sodium sulfate, filtered, and concentrated to dryness. The residue was prepared by high performance liquid chromatography (HPLC) with the following parameters: column: XBridge-1 5 μm 19-150 mm; mobile phase: acetonitrile-water (0.1% FA); gradient: 10-55/8 min to give the compound TPD12100 (48.8 mg, yellow solid, purity 98.482%) in the following yield: 9.92%. LCMS (ESI) m/z caled. for C₅₂H₅₃FN₁₀O₈ [M+H]⁺ 965.4; found 965.4; ¹H NMR (400 MHz, DMSO_d₆): δ=11.03 (s, 1H), 10.26 (s, 1H), 10.18 (s, 1H), 10.06 (s, 1H), 8.71 (s, 1H), 8.43 (d, J=5.2 Hz, 1H), 8.35 (s, 1H), 7.76 (d, J=8.9 Hz, 2H), 7.64 (dd, J=8.9, 5.1 Hz, 2H), 7.47 (s, 1H), 7.32 (s, 1H), 7.21 (d, J=8.9 Hz, 2H), 7.15 (t, J=8.9 Hz, 2H), 6.39 (d J=5.2 Hz, 1H), 5.11 (dd, J=13.2, 5.1 Hz, 1H), 4.56 (d, J=18.4 Hz, 1H), 4.41 (d, J=18.6 Hz, 1H), 3.95 (s, 4H), 3.60 (d, J=8.6 Hz, 5H), 2.88-2.54 (m, 9H), 2.37 (dd, J=17.8, 9.8 Hz, 4H), 2.04-1.97 (m, 1H), 1.88 (s, 2H), 1.71 (s, 2H), 1.47 (s, 5H). The compound structure is confirmed.

### Example 71: Synthesis of Compound TPD12101

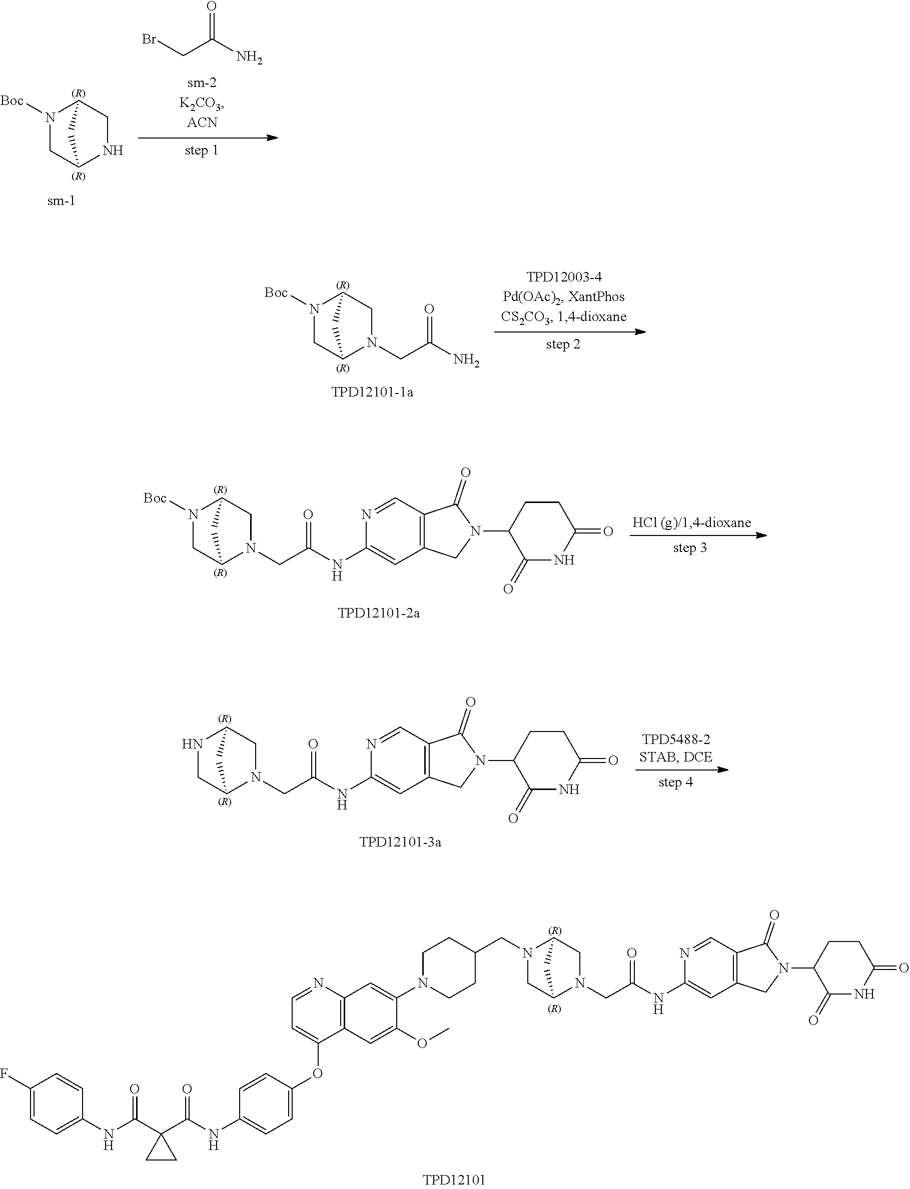

To a 25 ml single-necked vial were sequentially added compound sm-1 (500 mg, 2.51 mmol), 2-bromoacetamide (363 mg, 2.63 mmol), K₂CO₃ (520 mg, 3.76 mmol), and ACN (8 ml). The reaction was carried out under nitrogen protection at 25° C. for 16 hours. After the reaction was completed, the reaction solution was brought to room temperature, decanted into water (10 ml) and extracted three times with EA (30 ml). The organic phases were combined, washed with saturated brine, dried over anhydrous sodium sulfate, filtered, and concentrated to dryness to afford the compound TPD120101-1a (500 mg, white solid, purity 94.532%), yield: 73.49%. LCMS (ESI) m/z calcd. for C₁₂H₂₁N₃O₃ [M+H]⁺ 256.2; found 255.9; ¹H NMR (400 MHz, DMSO_d₆) 6=7.16 (d, J=46.2 Hz, 2H), 4.15 (d, J=12.1 Hz, 1H), 3.43 (s, 1H), 3.30 (s, 1H), 3.19-2.95 (m, 3H), 2.82 (dd, J=9.4, 1.7 Hz, 1H), 2.48 (s 1H), 1.75 (d, J=9.5 Hz, 1H), 1.65-1.55 (m, 1H), 1.39 (s, 9H). The compound structure is confirmed.

To a 25 ml single-necked vial was sequentially added compound TPD12101-1a (500 mg, 1.97 mmol), compound TPD12003-4 (550 mg, 1.97 mmol), palladium acetate (88 mg, 0.39 mmol), XantPhos (28 mg, 0.39 mmol), Cs₂ CO₃ (961 mg. 2.95 mmol) and 1,4-dioxane (15 ml). The reaction was carried out under nitrogen protection at 100° C. for 16 hours. After the reaction was completed, the reaction solution was reduced to room temperature, filtered with water (50 ml), the filter cake was washed with ethyl ester, the filtrate was extracted with ethyl ester (60 ml) three times, the organic phases were combined, washed with saturated brine, dried with anhydrous sodium sulfate, filtered, and concentrated to dryness, and the residue was purified by silica gel chromatography column with the ratio of the eluents as DCM/MeOH=100/1˜50/1. The crude product was purified by thin-layer chromatography plate. Purification, the unfolding agent ratio was DCM/MeOH=15/1 to obtain the compound TPD12101-2a (250 mg, yellow solid, purity 85.589%), yield: 21.78%. LCMS (ESI) m/z calcd. for C₂₄H₃₀N₆O₆ [M+H]⁺ 499.2; found 499.1; ¹H NMR (400 MHz, DMSO_d₆): δ=11.02 (s, 1H), 10.30 (s, 1H), 5.76 (s, 1H), 5.10 (dd, J=13.3, 5.1 Hz, 1H), 4.55 (d, J=18.5 Hz, 1H), 4.41 (d, J=18.5 Hz, 1H), 3.59 (s, 1H), 3.44 (t, J=10.3 Hz, 3.44 (t, J=10.3 Hz, 3H), 3.12-2.99 (m, 5H), 2.82 (dd, J=9.4, 1.7 Hz, 2H), 2.63 (d, J=9.4 Hz, 2H), 2.05-1.95 (m, 1H), 1.83 (d, J=9.7 Hz, 1H), 1.39 (s, 9H). The compound structure is confirmed.

To a 25 ml single-necked vial was sequentially added compound TPD12101-2a (250 mg, 0.50 mmol) and 4 N HCl/1,4-dioxane (8 ml). The reaction was carried out at 25° C. for 2 h. After completion of the reaction, it was concentrated to dryness to give the crude compound TPD12101-3a (250 mg, yellow solid, purity 79.403%), yield: 99.54%. LCMS (ESI) m/z calcd. for C₁₉H₂₂N₆O₄ [M+H]⁺ 399.2; found 399.2. The compound structure is confirmed.

To a 25 ml single-necked vial was sequentially added compound TPD12101-3a (125 mg, 0.31 mmol), compound TPD5488-2 (183 mg, 0.31 mmol), sodium triacetoxyborohydride (133 mg, 0.63 mmol) and DCE (6 ml). The reaction was carried out under nitrogen protection at 25° C. for 16 hours. After the reaction was completed, the reaction solution was poured into water (10 ml) and an appropriate amount of saturated NaHCO₃ solution was added to make the reaction solution alkaline. It was extracted three times with DCM (15 ml). The organic phases were combined, washed with saturated brine, dried over anhydrous sodium sulfate, filtered, and concentrated to dryness. The residue was prepared by high performance liquid chromatography (HPLC) with the following parameters: column: XBridge-1 5 μm 19-150 mm; mobile phase: acetonitrile-water (0.1% FA); gradient: 10-55/8 min to afford the compound TPD12101 (20.89 mg, yellow solid, purity 98.574%) in the following yield: 6.79%. LCMS (ESI) m/z calcd. for C₅₂H₅₃FN₁₀O₈ [M+H]⁺ 965.4; found 483.2, 965.4; ¹H NMR (400 MHz, DMSO_d₆): δ=11.03 (s, 1H), 10.27 (s, 1H), 10.19 (s, 1H), 10.07 (s, 1H), 8.71 (s, 1H). 8.43 (d, J=5.2 Hz, 1H), 8.35 (s, 1H), 8.20 (s, 2H), 7.76 (d, J=8.9 Hz, 2H), 7.64 (dd, J=9.0, 5.1 Hz, 2H), 7.47 (s, 1H), 7.32 (s, 1H), 7.25-7.10 (m, 4H), 6.39 (d, J=5.1 Hz, 1H), 5.11 (dd, J=13.2, 5.1 Hz, 1H), 4.56 (d, J=18.7 Hz, 1H), 4.42 (d, J=18.5 Hz, 1H), 3.95 (s, 3H), 3.60 (d, J=8.6 Hz, 2H), 3.44 (s, 4H), 3.05-2.74 (m 5H), 2.63 (s, 4H), 2.00 (d, J=5.2 Hz, 1H), 1.88 (s, 2H), 1.74 (s, 2H), 1.52-1.23 (m, 7H). The compound structure is confirmed.

### Example 72: Synthesis of Compound TPD12102

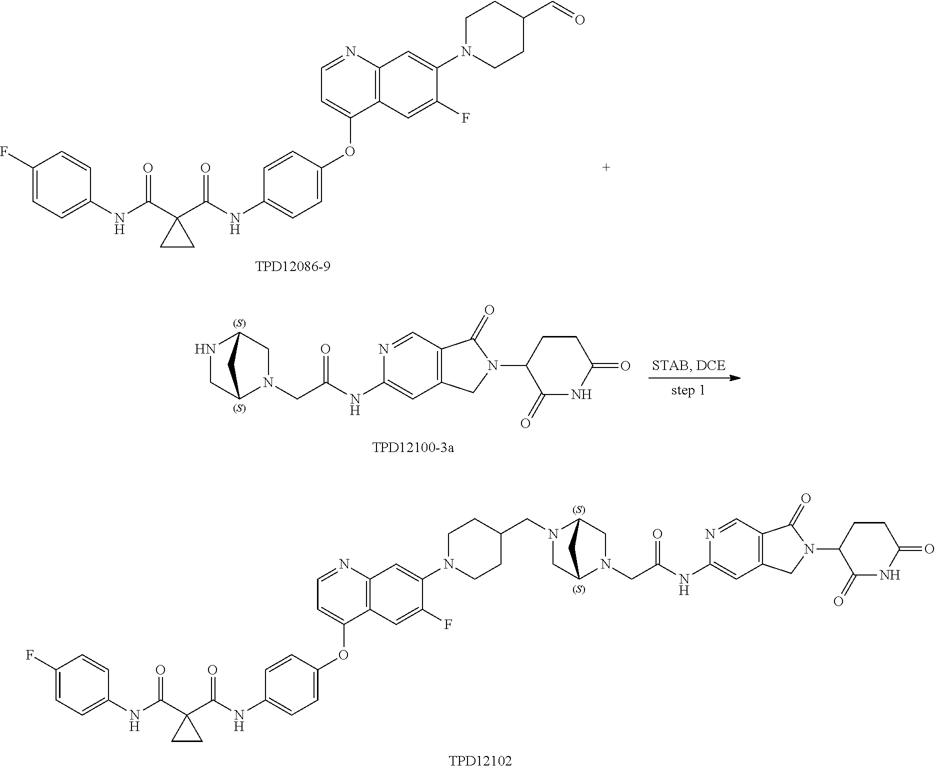

To a 25 ml single-necked vial was sequentially added compound TPD12100-3a (105 mg, 0.26 mmol), compound TPD12086-9 (150 mg, 0.26 mmol), sodium triacetoxyborohydride (111 mg, 0.53 mmol) and DCE (3 ml). The reaction was carried out under nitrogen protection at 25° C. for 16 hours. After the reaction was completed, the reaction solution was poured into water (10 ml) and an appropriate amount of saturated NaHCO₃ solution was added to make the reaction solution alkaline. It was extracted three times with DCM (5 ml). The organic phases were combined, washed with saturated brine, dried over anhydrous sodium sulfate, filtered, and concentrated to dryness. The residue was prepared by high performance liquid chromatography (HPLC) with the following parameters: column: XBridge-1 5 μm 19-150 mm; mobile phase: acetonitrile-water (0.1% FA); gradient: 10-55/8 min to afford the compound TPD12102 (23.1 mg, yellow solid, purity 96.732%), yield: 8.14%.

LCMS (ESI) m/z calcd. for C₅₁H₅₀F₂N₁₀O₇ [M+H]⁺ 953.38; found 953.3; ¹H NMR (400 MHz, DMSO_d₆): δ=11.02 (s, 1H), 10.23 (d, J=23.6 Hz, 2H), 10.06 (s, 1H), 8.71 (s, 1H). 8.56 (d, J=5.2 Hz, 1H), 8.35 (s, 1H), 7.86 (d, J=13.5 Hz, 1H), 7.76 (d, J=8.9 Hz, 2H), 7.64 (dd, J=9.0, 5.1 Hz, 2H), 7.45 (d, J=8.4 Hz, 1H), 7.22 (d, J=8.9 Hz, 2H), 7.15 (t, J=8.9 Hz, 2H), 6.45 (d, J=5.2 Hz, 1H), 5.11 (dd, J=13.2, 5.1 Hz, 1H), 4.56 (d, J=18.6 Hz, 1H), 4.41 (d, J=18.4 Hz, 1H), 3.58 (d, J=10.1 Hz, 2H), 3.42 (s, 3H), 2.95-2.84 (m, 2H), 2.77 (t, J=8.4 Hz, 3H), 2.69 (d, J=10.0 Hz, 1H), 2.65-2.53 (m, 2H), 2.48-2.30 (m, 4H), 2.03-1.96 (m, 1H), 1.92 (d, J=4.6 Hz, 2H). 1.70 (s, 2H), 1.58 (s, 1H), 1.47 (s, 4H), 1.36 (s, 2H). The compound structure is confirmed.

### Example 73: Synthesis of Compound TPD12110

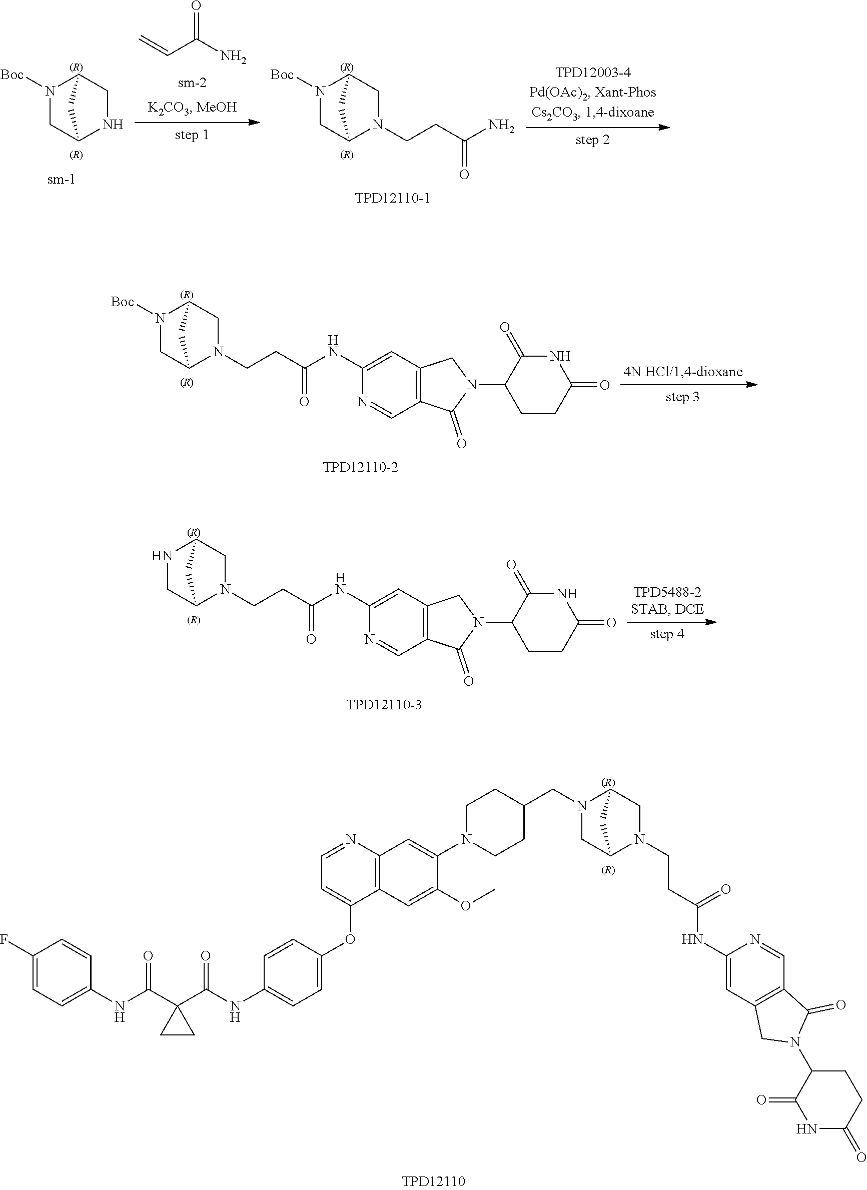

To a 50 ml single-necked vial was sequentially added compound sm-1 (300 mg, 1.51 mmol), acrylamide (139 mg, 1.96 mmol), potassium carbonate (312 mg, 2.26 mmol) and methanol (3 ml). The reaction was carried out under nitrogen protection at 25° C. for 16 hours. After completion of the reaction, the reaction solution was concentrated to dryness and the residue was purified by preparative plate with the unfolding agent ratio of DCM/MeOH=15/1 to afford the compound TPD12110-1 (400 mg, colorless oily substance) in the yield: 88.45%. LCMS (ESI) m/z calcd. for C₁₃H₂₃N₃O₃ [M+H]⁺ 270.2; found 270.1. The compound structure is confirmed.

To a 25 ml single-necked vial was sequentially added compound TPD12110-1 (300 mg, 1.11 mmol), compound TPD12003-4 (310 mg, 1.11 mmol), palladium acetate (50 mg, 0.22 mmol), XantPhos (128 mg, 0.22 mmol), cesium carbonate (542 mg, 1.66 mmol) and 1,4-dioxane (6 ml). The reaction was carried out under nitrogen protection at 100° C. for 16 hours. After completion of the reaction, the reaction solution was reduced to room temperature and concentrated to dryness. The residue was purified by column chromatography with eluent and ratio of DCM/MeOH=50/1˜30/1 to afford the compound TPD12110-2 (100 mg, yellow solid, purity 88.457%), yield: 11.64%. LCMS (ESI) m/z calcd. for C₂₅H₃₂N₆O₆ [M+H]⁺ 513.2; found 513.1; ¹H NMR (400 MHz, DMSO_d₆): δ=11.80 (s, 1H), 11.58 (s, 1H), 8.76 (s, 1H), 8.36 (s, 1H), 8.10 (s, 1H), 5.19 (dd, J=13.3, 5.1 Hz, 1H), 4.42 (dd, J=62.6, 16.8 Hz, 2H), 3.75-3.59 (m, 2H), 3.27 (d, J=10.1 Hz, 1H), 3.09-2.78 (m, 5H), 2.56-2.48 (m, 2H), 2.36 (qd, J=13.1, 4.8 Hz, 1H), 2.27-2.16 (m, 1H), 2.11-1.95 (m, 1H), 1.91-1.80 (m, 1H), 1.54-1.40 (m, 10H). The compound structure is confirmed.

Compound TPD12110-2 (100 mg, 0.19 mmol) and 4N HCl/1,4-dioxane (6 ml) were added sequentially in a 25 ml single-necked vial. The reaction was carried out under nitrogen protection at 25° C. for 2 hours. After completion of the reaction it was concentrated to dryness to give the crude compound TPD12110-3 (100 mg, off-white solid), yield: 96.97%. LCMS (ESI) m/z calcd. for C₂₀H₂₄N₆O₄ [M+H]⁺ 413.2; found 413.1. The compound structure is confirmed.

To a 25 ml single-necked vial was sequentially added compound TPD12110-3 (85 mg, 0.206 mmol), compound TPD5488-2 (120 mg, 0.206 mmol), sodium triacetoxyborohydride (87 mg, 0.412 mmol) and DCE (4 ml). The reaction was carried out under nitrogen protection at 20° C. for 16 hours. After the reaction was completed, the reaction solution was poured into saturated aqueous sodium bicarbonate solution (10 ml). The organic phase was separated and the aqueous phase was extracted three times with DCM (10 ml). The organic phases were combined, washed with saturated brine, dried over anhydrous sodium sulfate, filtered, and concentrated to dryness. The residue was separated by preparative plate (DCM/MeOH=10/1) to afford the compound TPD12110 (16.3 mg, yellow solid, purity 98.666%), yield: 7.09%. LCMS (ESI) m/z calcd. for C₅₃H₅₅FN₁₀O₈ [M+H]⁺ 979.4; found 490.2; 979.4; ¹H NMR (400 MHz, DMSO_d₆): δ=11.37-11.24 (m, 1H), 11.03 (s, 1H), 10.19 (s, 1H), 10.06 (s, 1H), 8.69 (s, 1H), 8.43 (d, J=5.2 Hz, 1H), 8.34 (s, 1H), 7.76 (d, J=8.7 Hz, 2H)), 7.64 (dd, J=8.7, 5.1 Hz, 2H), 7.47 (s, 1H), 7.32 (s, 1H), 7.27-7.10 (m, 4H), 6.39 (d, J=5.2 Hz, 1H), 5.10 (dd, J=13.2, 5.0 Hz, 1H), 4.47 (dd, J=58.2, 18.4 Hz 2H), 3.94 (s, 3H), 3.67-3.40 (m, 5H), 3.02-2.72 (m, 7H), 2.69-2.55 (m, 6H), 2.44-2.25 (m, 1H), 2.06-1.95 (m, 1H), 1.94-1.81 (m, 2H), 1.80-1.67 (m, 2H). 1.67-1.55 (m, 1H), 1.47 (s, 4H), 1.43-1.28 (m, 2H). The compound structure is confirmed.

### Example 74: Synthesis of Compound TPD12112

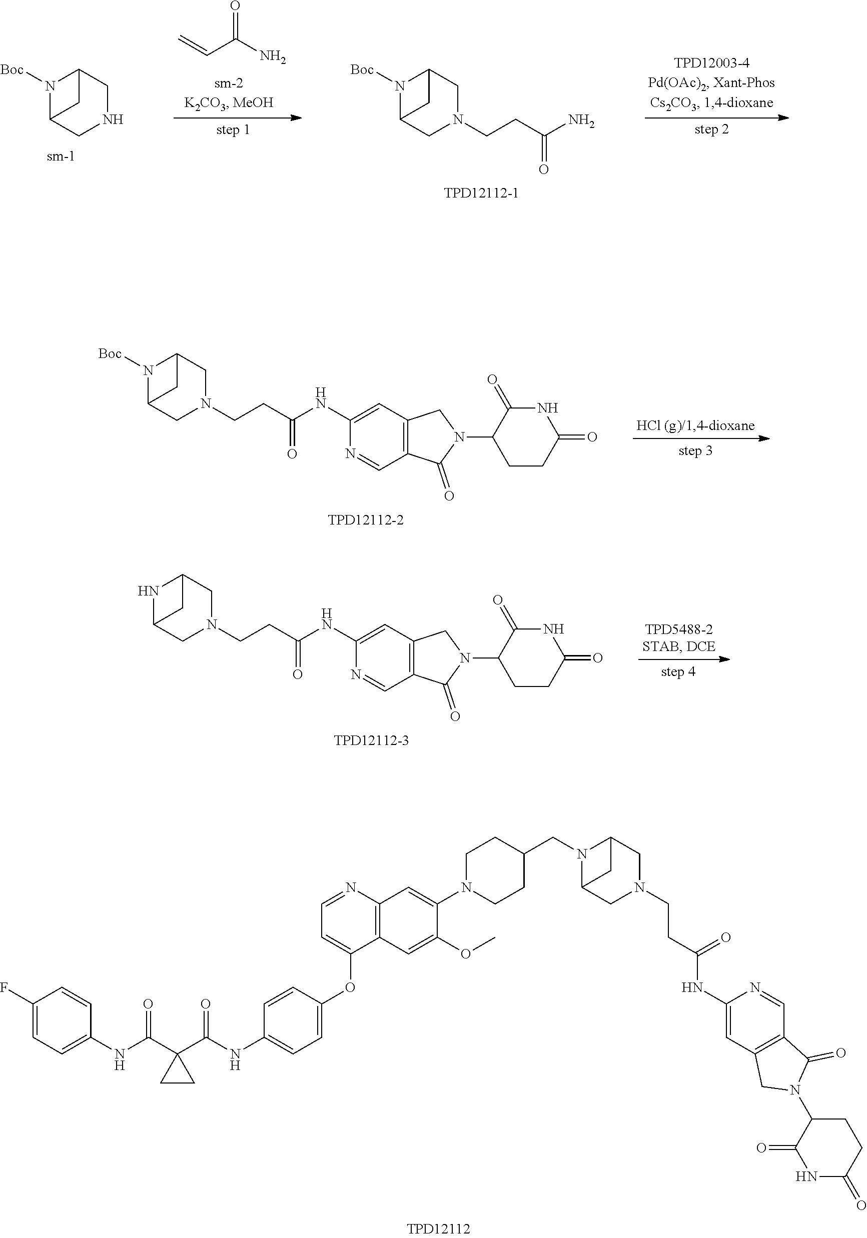

To a 50 ml single-necked vial was sequentially added compound sm-1 (500 mg, 2.51 mmol), acrylamide (232 mg, 3.26 mmol), potassium carbonate (520 mg, 3.76 mmol) and methanol (15 ml). The reaction was carried out under nitrogen protection at 25° C. for 16 hours. After the reaction was completed, the reaction solution was poured into water (50 ml) and extracted three times with EA (50 ml). The organic phases were combined, washed with saturated brine, dried over anhydrous sodium sulfate, filtered, and concentrated to dryness, and the residue was purified by silica gel chromatographic column with eluent ratio of DCM/MeOH=100/1˜30/1 to afford the compound TPD12112-1 (340 mg, white solid, purity 97.539%) in the yield: 48.89%. LCMS (ESI) m/z calcd. for C₁₃H₂₃N₃O₃ [M+H]⁺ 270.2; found 269.9; ¹H NMR (400 MHz, DMSO_d₆): δ=7.33 (s, 1H), 6.76 (s, 1H), 3.94 (d, J=5.8 Hz, 2H), 3.00-2.89 (m, 2H), 2.78-2.66 (m, 4H). 2.29-2.19 (m, 3H), 1.52 (d, J=7.9 Hz, 1H), 1.38 (s, 9H). The compound structure is confirmed.

To a 25 ml single-necked vial was sequentially added compound TPD12112-1 (300 mg, 1.11 mmol), compound TPD12003-4 (310 mg, 1.11 mmol), palladium acetate (50 mg, 0.22 mmol), XantPhos (128 mg, 0.22 mmol), cesium carbonate (542 mg, 1.66 mmol) and 1,4-dioxane (5 ml). The reaction was carried out under nitrogen protection at 100° C. for 16 hours. After completion of the reaction, the reaction solution was brought to room temperature, decanted into water (30 ml) and extracted three times with EA (20 ml). The organic phases were combined, washed with saturated brine, dried over anhydrous sodium sulfate, filtered, and concentrated to dryness. The residue was purified by preparative plate with unfolding agent ratio DCM/MeOH=15/1 to afford compound TPD12112-2 (90 mg, brown solid, purity 98.588%), yield: 15.57%. LCMS (ESI) m/z calcd. for C₂₅H₃₂N₆O [M+H]⁺ 513.2; found 513.2; ¹H NMR (400 MHz, DMSO_d₆): δ=11.07 (s, 1H), 11.01 (s, 1H), 8.67 (s, 1H), 8.33 (s, 1H), 5.10 (dd, J=13.3, 5.1 Hz, 1H), 4.53 (d, J=18.4 Hz, 1H), 4.38 (d, J=18.4 Hz, 1H). 3.97 (d, J=5.7 Hz, 2H), 2.95-2.81 (m, 7H), 2.63-2.59 (m, 3H), 2.43-2.33 (m, 1H), 2.28 (dd, J=13.5, 6.2 Hz, 1H), 2.02-1.97 (m, 1H), 1.61 (d, J=7.9 Hz, 1H). 1.38 (s, 9H). The compound structure is confirmed.

To a 25 ml single-necked vial was sequentially added compound TPD12112-2 (90 mg, 0.18 mmol), 1,4-dioxane (1 ml) and 4N HCl/1,4-dioxane (4 ml). The reaction was carried out under nitrogen protection at 25° C. for 2 hours. After completion of the reaction it was concentrated to dryness to give the crude compound TPD12112-3 (100 mg, off-white solid), yield: 111.99%. LCMS (ESI) m/z calcd. for C₂₀H₂₄N₆O₄ [M+H]⁺ 413.2; found 413.1. The compound structure is confirmed.

To a 25 ml single-necked vial was sequentially added compound TPD12112-3 (100 mg, 0.24 mmol), compound TPD5488-2 (141 mg, 0.24 mmol), sodium triacetoxyborohydride (103 mg, 0.485 mmol) and DCE (3 ml). The reaction was carried out under nitrogen protection at 25° C. for 16 hours. After the reaction was completed, the reaction solution was decanted into water (15 ml) and an appropriate amount of saturated NaHCO₃ solution was added to make the reaction solution alkaline. The organic phase was separated and the aqueous phase was extracted twice with DCM (10 ml). The organic phases were combined, washed with saturated brine, dried over anhydrous sodium sulfate, filtered, and concentrated to dryness. The residue was prepared by high performance liquid chromatography (HPLC) with the following parameters: column: T3 5 μm 19-150 mm; mobile phase: acetonitrile-water (0.1% FA); gradient: 20-35/7 min to afford the compound TPD12112 (21.3 mg, yellow solid, purity 97.980%) in the following yield: 8.25%. LCMS (ESI) m/z calcd. for C₅₃H₅₅FN₁₀O₈ [M+H]⁺ 979.4; found 490.3; 979.3; ¹H NMR (400 MHz, DMSO_d₆): δ=11.18 (s, 1H), 11.04 (s, 1H), 10.19 (s, 1H), 10.06 (s, 1H), 8.68 (s, 1H), 8.43 (d J=5.2 Hz, 1H), 8.36 (s, 1H), 8.21 (s, 1.4H), 7.76 (d, J=8.8 Hz, 2H), 7.64 (dd, J=8.9, 5.1 Hz, 2H), 7.46 (s, 1H), 7.31 (s, 1H), 7.21 (d, J=8.9 Hz, 2H), 7.15 (t, J=8.9 Hz, 2H), 6.39 (d, J=5.2 Hz, 1H), 5.09 (dd, J=13.3, 5.0 Hz, 1H), 4.53 (d, J=18.5 Hz, 1H), 4.39 (d, J=18.4 Hz, 1H), 3.94 (s, 3H), 3.56 (d, J=12.3 Hz, 5H), 2.99 (dd, J=17.8, 8.9 Hz, 4H), 2.93-2.85 (m, 3H), 2.67-2.58 (m, 5H), 2.41-2.33 (m, 4H), 1.99-1.94 (m, 1H), 1.89 (d, J=7.9 Hz, 1H), 1.80 (d, J=11.2 Hz, 2H), 1.47 (s 4H), 1.40-1.32 (m, 2H). The compound structure is confirmed.

### Example 75: Synthesis of Compound TPD12113

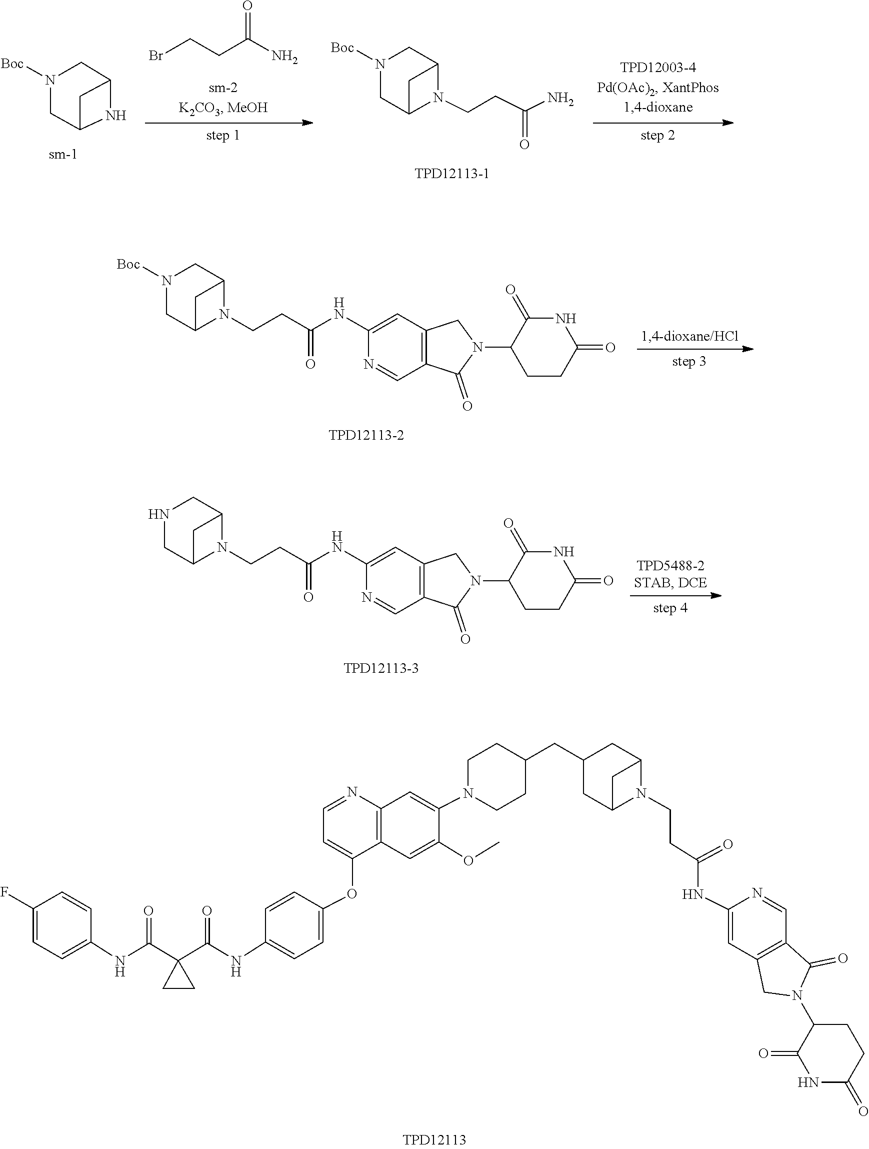 [SMILES: CC(C)(C)OC(=O)N1CC2CC(C1)N2 ; NC(=O)CCBr ; CC(C)(C)OC(=O)N1CC2CC(C1)N2CCC(N)=O ; CC(C)(C)OC(=O)N1CC2CC(C1)N2CCC(=O)Nc1cc2c(cn1)C(=O)N(C1CCC(=O)NC1=O)C2 ; O=C1CCC(N2Cc3cc(NC(=O)CCN4C5CNCC4C5)ncc3C2=O)C(=O)N1 ; COc1cc2c(Oc3ccc(NC(=O)C4(C(=O)Nc5ccc(F)cc5)CC4)cc3)ccnc2cc1N1CCC(CC2CC3CC(C2)N3CCC(=O)Nc2cc3c(cn2)C(=O)N(C2CCC(=O)NC2=O)C3)CC1]

To a 25 ml single-necked vial were sequentially added compound sm-1 (600 mg, 3.01 mmol), 3-bromopropionamide (595 mg, 3.91 mmol), K₂CO₃ (624 mg, 4.52 mmol), and ACN (15 ml). The reaction was carried out under nitrogen protection at 80° C. for 16 hours. After the reaction was completed, the reaction solution was brought to room temperature, decanted into water (10 ml) and extracted three times with EA (30 ml). The organic phases were combined, washed with saturated brine, dried over anhydrous sodium sulfate, filtered, and concentrated to dryness to afford the compound TPD120113-1 (650 mg, white solid, purity 74.165%), yield: 59.22%. LCMS (ESI) m/z calcd. for C₁₃H₂₃N₃O₃ [M+H]⁺ 270.2; found 269.9; ¹H NMR (400 MHz, DMSO_d₆): δ=6.75 (s, 1H), 3.54-3.38 (m, 6H), 3.24-3.16 (m, 2H), 2.47 (s, 1H), 2.32 (dd, J=13.8, 6.0 Hz, 1H), 2.06 (t, J=7.1 Hz, 2H), 1.42 (s, 9H). The compound structure is confirmed.

To a 25 ml single-necked vial was sequentially added compound TPD12113-1 (650 mg, 2.32 mmol), compound TPD12003-4 (628 mg, 2.32 mmol), palladium acetate (104 mg, 0.46 mmol), XantPhos (268.94 mg, 0.46482 mmol), Cs₂ CO₃ (1136 mg, 3.49 mmol) and 1,4-dioxane (15 ml). The reaction was carried out under nitrogen protection at 100° C. for 16 hours. After the reaction was completed, the reaction solution was reduced to room temperature, filtered with water (50 ml), the filter cake was washed with ethyl ester, the filtrate was extracted with ethyl ester (60 ml) three times, the organic phases were combined, washed with saturated brine, dried with anhydrous sodium sulfate, filtered, and concentrated to dryness, and the residue was purified by silica gel chromatography column with the ratio of the eluents as DCM/MeOH=100/1˜50/1. The crude product was purified by thin-layer chromatography plate. Purification, the unfolding agent ratio was DCM/MeOH=15/1 to obtain the compound TPD12113-2 (250 mg, yellow solid, purity 94.269%), yield: 21.32%. LCMS (ESI) m/z calcd. for C₂₅H₃₂N₆O₆ [M+H]⁺ 513.2; found 513.3; ¹H NMR (400 MHz. DMSO_d₆): δ 11.03 (s, 2H), 8.69 (s, 1H), 8.33 (s, 1H), 5.75 (s, 1H), 5.09 (dd, J=13.3, 5.1 Hz, 1H), 4.59-4.33 (m, 2H), 3.53 (dd, J=20.2, 12.3 Hz, 4H), 3.28-3.20 (m, 2H), 2.98-2.86 (m, 1H), 2.72-2.57 (m, 3H), 2.46-2.26 (m, 3H), 2.04-1.95 (m, 1H), 1.43 (s, 9H). The compound structure is confirmed.

To a 25 ml single-necked vial were sequentially added compound TPD12113-2 (270 mg, 0.53 mmol) and 4N HCl/1,4-dioxane (8 ml). The reaction was carried out at 25° C. for 2 h. After completion of the reaction, it was concentrated to dryness to give the crude compound TPD12113-3 (270 mg, yellow solid, purity 94.118%), yield: 117.2.%. LCMS (ESI) m/z calcd. for C₂₀H₂₄N₆O₄ [M+H]⁺ 413.2; found 413.1. as seen. the structure of the compound is correct.

To a 25 ml single-necked vial were sequentially added compound TPD12113-3 (135 mg, 0.33 mmol), compound TPD5488-2 (191 mg, 0.3273 mmol), sodium triacetoxyborohydride (139 mg, 0.65 mmol) and DCE (6 ml). The reaction was carried out under nitrogen protection at 25° C. for 16 hours. After the reaction was completed, the reaction solution was poured into water (15 ml) and an appropriate amount of saturated NaHCO₃ solution was added to make the reaction solution alkaline. It was extracted three times with DCM (15 ml). The organic phases were combined, washed with saturated brine, dried over anhydrous sodium sulfate, filtered, and concentrated to dryness. The residue was prepared by high performance liquid chromatography (HPLC) with the following parameters: column: XBridge-1 5 μm 19-150 mm; mobile phase: acetonitrile-water (0.1% FA); gradient: 10-55/8 min to afford the compound TPD12113 (7.46 mg, yellow solid, purity 97.940%) in the following yield: 2.29%. LCMS (ESI) m/z calcd. for C₅₃H₅₅FN₁₀O₈ [M+H]⁺ 979.4; found 490.3, 979.4; 1H NMR (400 MHz, DMSO_d₆): δ=11.19 (s, 1H), 11.02 (s, 1H), 10.19 (s, 1H), 10.07 (s, 1H), 8.69 (s, 1H). 8.43 (d, J=5.1 Hz, 1H), 8.34 (s, 1H), 8.22 (s, 2H), 7.76 (d, J=8.9 Hz, 2H), 7.64 (dd, J=8.9, 5.1 Hz, 2H), 7.46 (s, 1H), 7.32 (s, 1H), 7.23-7.12 (m, 4H), 6.39 (d, J=5.2 Hz, 1H), 5.10 (dd, J=13.2, 5.1 Hz, 1H), 4.54 (d, J=18.3 Hz, 1H), 4.39 (d, J=18.6 Hz, 1H), 3.94 (s, 3H), 3.17 (s, 1H), 2.87-2.68 (m, 5H), 2.79-2.53 (m, 6H). 2.33-2.40 (m, 4H), 2.09-1.94 (m, 2H), 1.91-1.68 (m, 5H), 1.47 (s, 4H), 1.34-1.23 (m, 3H). The compound structure is confirmed.

### Example 76: Synthesis of Compound TPD12116

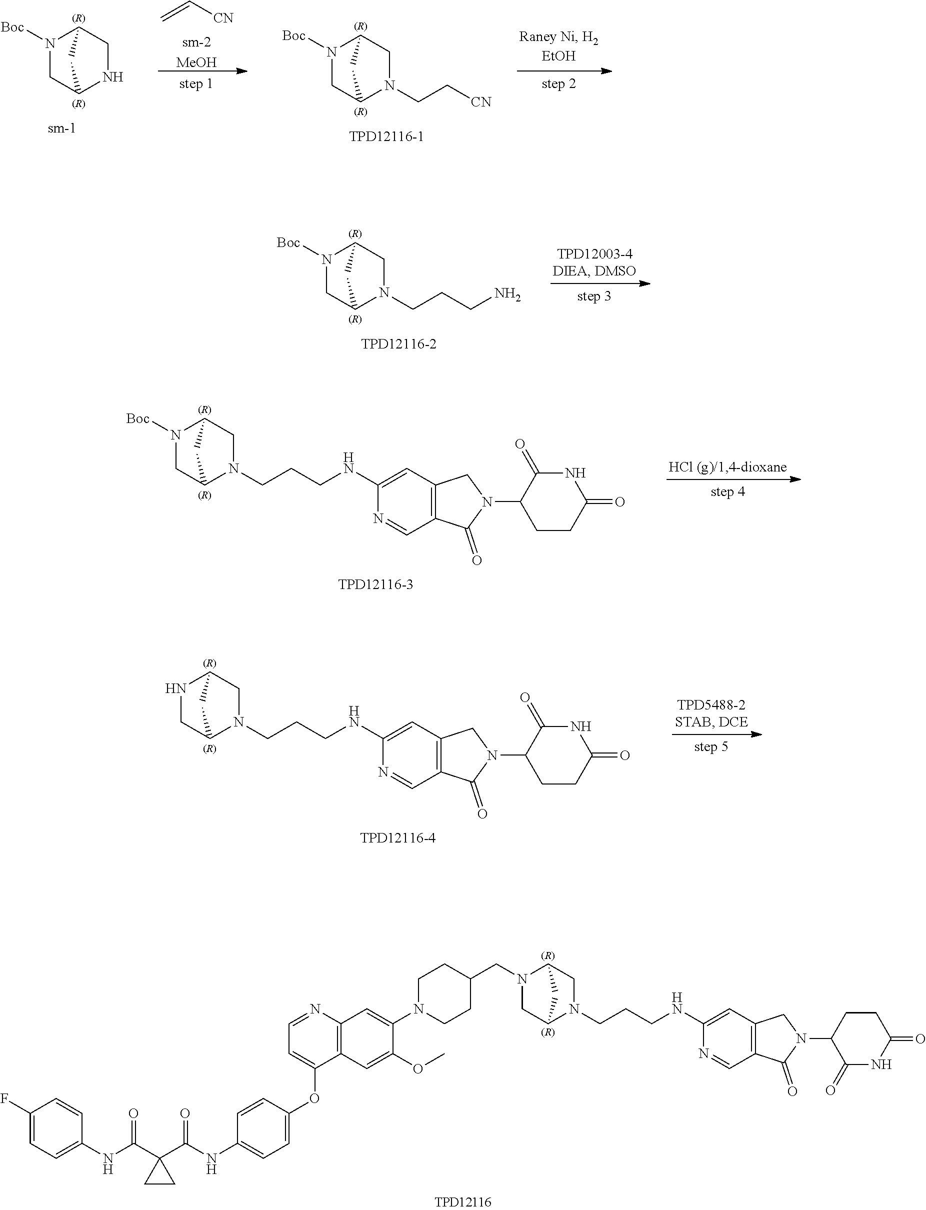

Compound sm-1 (500 mg, 2.51 mmol), acrylonitrile (160 mg, 3.01 mmol) and methanol (10 ml) were added sequentially in a 50 ml single-necked vial. The reaction was carried out under nitrogen protection at 25° C. for 16 hours. After the reaction was completed, the reaction solution was poured into water (50 ml) and extracted three times with EA (10 ml). The organic phases were combined, washed with saturated brine, dried over anhydrous sodium sulfate, filtered, and concentrated to dryness, and the residue was purified by silica gel chromatographic column with eluent ratio of DCM/MeOH=100/1˜30/1 to afford the compound TPD12116-1 (560 mg, colorless oil, purity 93.352%) in the yield: 82.57%. LCMS (ESI) m/z calcd. for C₁₃H₂₁N₃O₂ [M+H]⁺ 252.2; found 251.8; ¹H NMR (400 MHz, DMSO_d₆): δ=4.14 (d, J=9.9 Hz, 1H), 3.51 (s, 1H), 3.25 (dd, J=10.0, 5.2 Hz, 2H), 3.10-3.04 (m, 1H). 2.82-2.79 (m, 1H), 2.74-2.68 (m, 2H), 2.58 (t, J=6.5 Hz, 2H), 1.71-1.59 (m, 2H), 1.39 (s, 9H). The compound structure is confirmed.

To a 100 ml single-necked vial were sequentially added the compounds TPD12116-1 (560 mg, 2.22 mmol), EtOH/NH H₃₂ O=10/1 (10 ml) and Raney nickel (130 mg, 2.22 mmol). The reaction was carried out for 16 h at 40° C. under the protection of hydrogen (1 atm). After completion of the reaction, the reaction solution was filtered and concentrated to dryness to give the crude compound TPD12116-2 (530 mg, colorless oil, purity 83.869%), yield: 78.13%. LCMS (ESI) m/z calcd. for C₁₃H₂₅N₃O₂ [M+H]⁺ 256.2; found 255.8. The compound structure is confirmed.

To a 25 ml three-necked vial was sequentially added compound TPD12116-2 (500 mg, 1.95 mmol), compound TPD12003-4 (545 mg, 1.95 mmol), DIEA (504 mg, 3.90 mmol) and DMSO (5 ml). The reaction was carried out under nitrogen protection at 130° C. for 16 hours. After completion of the reaction, the reaction solution was reduced to room temperature, decanted into water (30 ml) and extracted three times with EA (10 ml). The organic phases were combined, washed with saturated brine, dried over anhydrous sodium sulfate, filtered, and concentrated to dryness. The residue was purified by preparative plate with the unfolding agent ratio of DCM/MeOH=8/1 to afford the compound TPD12116-3 (200 mg, brown solid, purity 83.106%), yield: 17.06%. LCMS (ESI) m/z calcd. for C₂₅H₃₄N₆O₅ [M+H]⁺ 499.2; found 499.1; ¹H NMR (400 MHz, DMSO_d₆): δ=10.97 (s, 1H), 8.35 (s, 1H), 7.21 (t, J=5.0 Hz, 1H), 6.54 (s, 1H), 5.02 (dd, J=13.3, 5.0 Hz, 1H), 4.32 (d, J=17.3 Hz, 1H), 4.20-4.12 (m, 2H)), 3.35-3.30 (m, 4H), 3.05 (t, J=11.3 Hz, 1H), 2.94-2.79 (m, 2H), 2.60-2.53 (m, 2H), 2.42-2.27 (m, 3H), 1.96-1.93 (m, 1H), 1.71 (d, J=9.1 Hz, 1H), 1.65-1.58 (m, 3H), 1.39 (s, 9H). The compound structure is confirmed.

To a 25 ml single-necked vial was sequentially added compound TPD12116-3 (200 mg, 0.40 mmol), 1,4-dioxane (1 ml) and 4N HCl/1,4-dioxane (4 ml). The reaction was carried out under nitrogen protection at 25° C. for 2 hours. After completion of the reaction it was concentrated to dryness to give the crude compound TPD12116-4 (200 mg, off-white solid), yield: 90.98%. LCMS (ESI) m/z calcd. for C₂₀H₂₆N₆O [M+H]⁺ 399.2; found 399.1. The compound structure is confirmed.

To a 25 ml single-necked vial was sequentially added compound TPD12116-4 (100 mg, 0.25 mmol), compound TPD5488-2 (146 mg, 0.25 mmol), sodium triacetoxyborohydride (106 mg, 0.50 mmol) and DCE (3 ml). The reaction was carried out under nitrogen protection at 25° C. for 16 hours. After the reaction was completed, the reaction solution was decanted into water (15 ml) and an appropriate amount of saturated NaHCO₃ solution was added to make the reaction solution alkaline. The organic phase was separated and the aqueous phase was extracted twice with DCM (10 ml). The organic phases were combined, washed with saturated brine, dried over anhydrous sodium sulfate, filtered, and concentrated to dryness. The residue was prepared by high performance liquid chromatography (HPLC) with the following parameters: column: sunfire 5 μm 19-150 mm; mobile phase: acetonitrile-water (0.1% FA); gradient: 5-40/7 min to afford compound TPD12116 (4.65 mg, yellow solid, purity 98.518%) in 1.71% yield. LCMS (ESI) m/z calcd. for C₅₃H₅₇FN₁₀O₇ [M+H]⁺ 965.4; found 483.3; 966.4; ¹H NMR (400 MHz, DMSO_d₆): δ=11.00 (s, 1H), 10.20 (s, 1H), 10.07 (s, 1H), 8.43 (d, J=5.2 Hz, 1H), 8.36 (s, 1H). 8.25 (s, 2H), 7.76 (d, J=8.8 Hz, 2H), 7.64 (dd, J=8.9, 5.1 Hz, 2H), 7.46 (s, 1H), 7.31 (d, J=16.8 Hz, 2H), 7.21 (d, J=8.9 Hz, 2H), 7.15 (t, J=8.9 Hz, 2H), 6.57 (s 1H), 6.39 (d, J=5.2 Hz, 1H), 5.03 (dd, J=13.3, 5.1 Hz, 1H), 4.33 (d, J=17.3 Hz, 2H), 4.19 (d, J=17.3 Hz, 2H), 3.94 (s, 3H), 3.59 (d, J=11.3 Hz, 5H), 3.44 (s, 1H). 3.36 (s, 2H), 2.94-2.80 (m, 5H), 2.74 (d, J=9.1 Hz, 1H), 2.66-2.60 (m, 3H), 2.38-2.27 (m, 2H), 1.95-1.92 (m, 1H), 1.85 (d, J=10.7 Hz, 2H), 1.76 (s, 3H), 1.55 (br. s, 1H), 1.47 (s, 4H), 1.35 (br. s, 2H). The compound structure is confirmed.

### Example 77: Synthesis of compound TPD12118

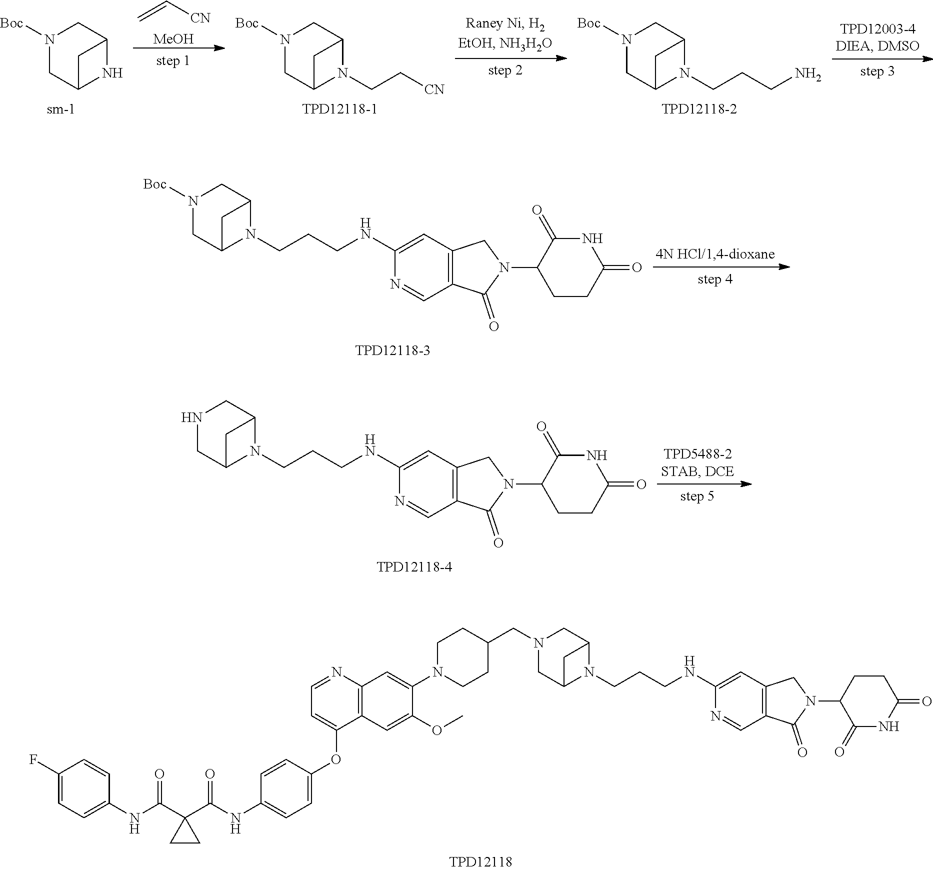

To a 100 ml single-necked vial were sequentially added compound sm-1 (1 g, 0.005 mol), acrylonitrile (0.27 g, 0.005 mol) and MeOH (10 ml). The reaction was carried out under nitrogen protection at 65° C. for 16 hours. After completion of the reaction, the reaction solution was reduced to room temperature, decanted into water (30 ml) and extracted three times with DCM (20 ml). The organic phases were combined, washed with saturated brine, dried over anhydrous sodium sulfate, filtered, and concentrated to dryness to afford the compound TPD12118-1 (0.9 g, white solid, purity 79.943%), yield: 56.00%. LCMS (ESI) m/z calcd. for ₁₃H₂₁N₃O₂ [M+H]⁺ 252.16; found 252.1; ¹H NMR (400 MHz, DMSO_d₆): δ=3.59-3.40 (m, 4H), 3.23 (dd, J=18.2, 12.1 Hz, 2H), 2.52 (d, J=13.1 Hz, 5H), 2.38 (d, J=7.5 Hz, 1H), 1.43 (s, 9H). The compound structure is confirmed.

To a 100 ml single-necked vial were sequentially added compounds TPD12118-1 (0.9 g, 0.0036 mol), Raney Ni (0.63 g, 0.0108 mol), EtOH (20 ml) and NH H₃₂ O (2 ml). The reaction was carried out for 8 hours at 40° C. under hydrogen protection. After completion of the reaction, the reaction solution was reduced to room temperature, filtered and concentrated to dryness. The residue was purified by silica gel column with eluent ratio of DCM/MeOH=50/1˜30/1 to obtain the compound TPD12118-2 (0.7 g, yellow oil, purity 81.874%), yield: 61.11%. LCMS (ESI) m/z calcd. for C₁₃H₂₅N₃O₂ [M+H]⁺ 256.19; found 256.1; ¹H NMR (400 MHz, CDCl₃): δ=7.27 (s, 1H), 3.56 (s, 2H), 3.35 (dd, J=18.5, 6.3 Hz, 3H), 2.77 (t, J=6.8 Hz, 2H), 2.47 (t, J=7.2 Hz, 2H), 1.75 (s, 6H), 1.49 (d, J=4.2 Hz, 9H). The compound structure is confirmed.

Compound TPD12118-2 (500 mg, 2.06 mmol), compound TPD12003-4 (582 mg, 2.27 mmol), DIEA (400 mg, 3.09 mmol), and DMSO (6 ml) were added sequentially in a 25 ml single-necked vial. The reaction was carried out under nitrogen protection at 130° C. for 16 hours. After completion of the reaction, the reaction solution was reduced to room temperature, decanted into water (30 ml) and extracted three times with EA (30 ml). The organic phases were combined, washed with saturated brine, dried over anhydrous sodium sulfate, filtered, and concentrated to dryness. The residue was purified by preparative plate with the unfolding agent ratio of DCM/MeOH=8/1 to afford the compound TPD12118-3 (65 mg, yellow solid, purity 93.786%), yield: 5.91%. LCMS (ESI) m/z calcd. for C₂₅H₃₄N₆O₅ [M+H]⁺ 499.26; found 499.1; ¹H NMR (400 MHz, DMSO_d₆): δ=10.96 (s, 1H), 8.34 (s, 1H), 7.21 (d, J=7.1 Hz, 1H), 6.54 (s, 1H), 5.76 (s, 2H), 5.02 (dd, J=13.3, 5.0 Hz, 1H), 4.24 (dd, J=58.0, 17.4 Hz, 2H), 3.43 (dd, J=29.2, 10.7 Hz, 3H), 3.19 (d, J=12.3 Hz, 2H), 2.90-2.82 (m, 1H), 2.63 (d, J=30.7 Hz, 2H), 2.34 (dd, J=14.9, 10.0 Hz, 4H), 1.95 (s, 2H), 1.58 (d, J=7.3 Hz, 2H), 1.44-1.38 (m, 9H). The compound structure is confirmed.

To a 25 ml single-necked vial was sequentially added compound TPD12118-3 (65 mg, 0.13 mmol) and 4N HCl/1,4-dioxane (3 ml). The reaction was carried out at 25° C. for 2 h. After completion of the reaction, it was concentrated to dryness to give the crude compound TPD12118-4 (80 mg, yellow solid), yield: 77.09%. LCMS (ESI) m/z calcd. for C₂₀H₂₆N₆O₃ [M+H]⁺ 399.21; found 399.0. The compound structure is confirmed.

To a 25 ml single-necked vial were sequentially added compound TPD12118-4 (80 mg, 0.20 mmol), compound TPD5488-2 (117 mg, 0.20 mmol), sodium triacetoxyborohydride (85 mg, 0.40 mmol) and DCE (2 ml). The reaction was carried out under nitrogen protection at 25° C. for 16 hours. After the reaction was completed, the reaction solution was poured into water (6 ml) and an appropriate amount of saturated NaHCO₃ solution was added to make the reaction solution alkaline. It was extracted three times with DCM (5 ml). The organic phases were combined, washed with saturated brine, dried over anhydrous sodium sulfate, filtered, and concentrated to dryness. The residue was prepared by high performance liquid chromatography (HPLC) with the following parameters: column: XBridge-1 5 μm 19-150 mm; mobile phase: acetonitrile-water (0.1% FA); gradient: 10-55/8 min to afford the compound TPD12118 (6.21 mg, yellow solid, purity 98.746%) in the following yield: 2.89%. LCMS (ESI) m/z calcd. for C₅₃H₅₇FN₁₀O₇ [M+H]⁺ 965.44; found 965.4; ¹H NMR (400 MHz, CD₃ OD): δ=8.57-8.46 (m, 2H), 7.81 (d, J=8.9 Hz, 2H), 7.76 (s, 1H), 7.56 (dd, J=8.9, 4.8 Hz, 2H). 7.36-7.28 (m, 3H), 7.07 (t, J=8.7 Hz, 2H), 6.80 (d, J=6.6 Hz, 2H), 5.09 (dd, J=13.4, 5.0 Hz, 1H), 4.55-4.30 (m, 4H), 4.09 (s, 3H), 3.94 (d, J=12.2 Hz, 2H), 3.62-3.36 (m, 5H), 3.30-3.13 (m, 4H), 2.97-2.58 (m, 7H), 2.49-2.35 (m, 2H), 2.04 (dd, J=75.8, 6.6 Hz, 7H), 1.65 (s, 5H), 1.44 (d, J=30.5 Hz, 2H), 1.30 (d, J=8.9 Hz, 1H). The compound structure is confirmed.

### Example 78: Synthesis of Compound TPD12139

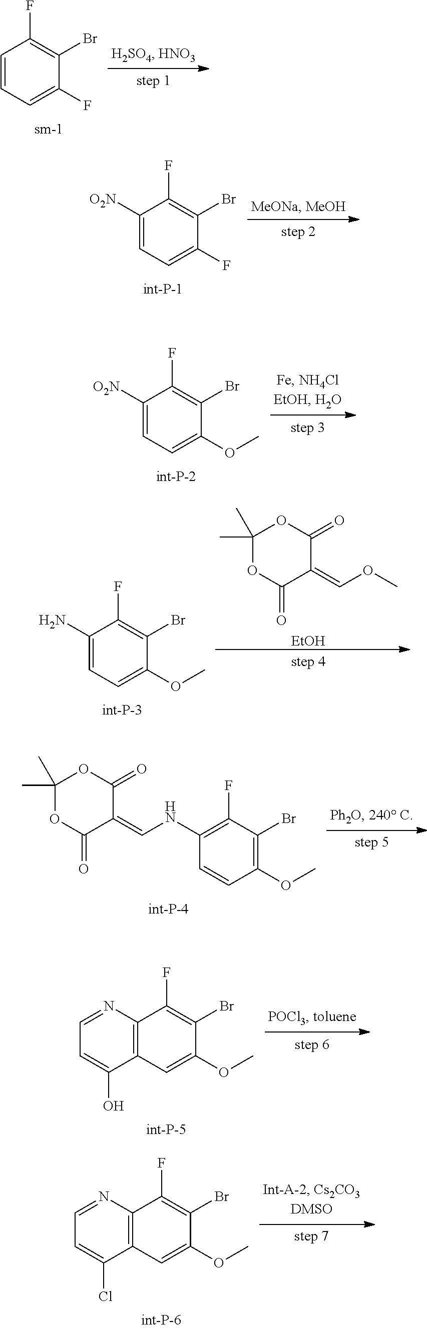

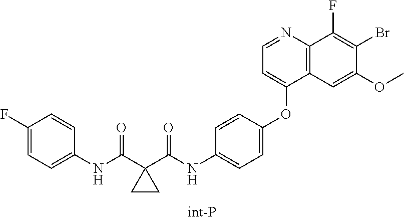 [SMILES: COc1cc2c(Oc3ccc(NC(=O)C4(C(=O)Nc5ccc(F)cc5)CC4)cc3)ccnc2c(F)c1Br]

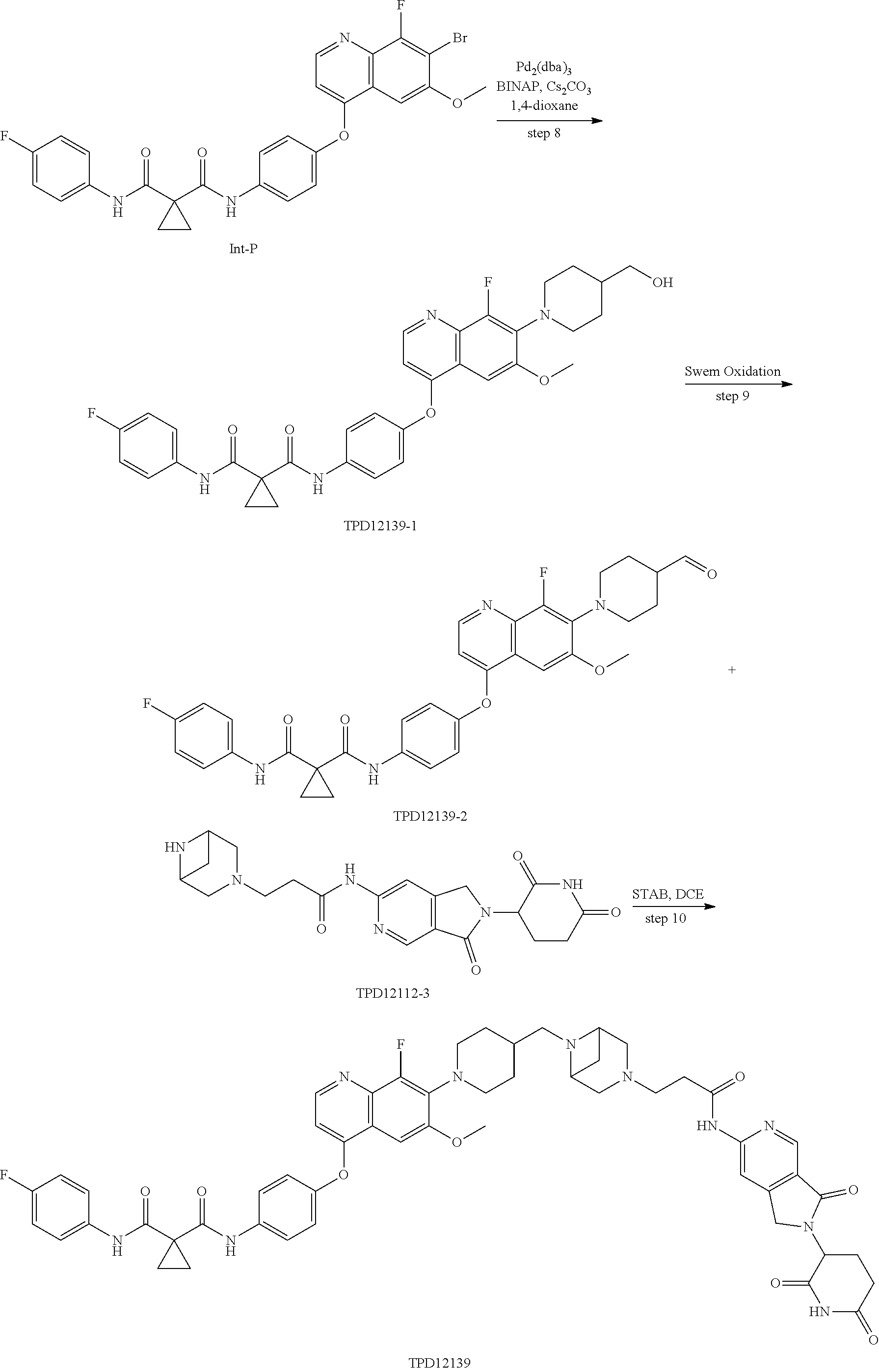 [SMILES: COc1cc2c(Oc3ccc(NC(=O)C4(C(=O)Nc5ccc(F)cc5)CC4)cc3)ccnc2c(F)c1Br ; COc1cc2c(Oc3ccc(NC(=O)C4(C(=O)Nc5ccc(F)cc5)CC4)cc3)ccnc2c(F)c1N1CCC(CO)CC1 ; COc1cc2c(Oc3ccc(NC(=O)C4(C(=O)Nc5ccc(F)cc5)CC4)cc3)ccnc2c(F)c1N1CCC(C=O)CC1 ; O=C1CCC(N2Cc3cc(NC(=O)CCN4CC5CC(C4)N5)ncc3C2=O)C(=O)N1 ; COc1cc2c(Oc3ccc(NC(=O)C4(C(=O)Nc5ccc(F)cc5)CC4)cc3)ccnc2c(F)c1N1CCC(CN2C3CC2CN(CCC(=O)Nc2cc4c(cn2)C(=O)N(C2CCC(=O)NC2=O)C4)C3)CC1]

To a 250 ml triple-necked flask was sequentially added compound sm-1 (25 g, 0.1295 mol), concentrated sulfuric acid (125 ml) and concentrated nitric acid (12.5 ml). The reaction was carried out under nitrogen protection at 50° C. for 2 hours. After the reaction was completed, the reaction solution was reduced to room temperature, adjusted to weak basicity with NaOH solution and extracted three times with DCM (100 ml). The organic phases were combined, washed with saturated brine, dried over anhydrous sodium sulfate, filtered, and concentrated to dryness, and the residue was purified by silica gel chromatographic column with eluent ratio of PE/EtOAc=20/1˜5/1 to obtain the compound int-P-1 (15 g, off-white solid, purity 99.721%), yield: 48.57%. LCMS (ESI) m/z calcd. for C₆H₂BrF₂NO₂ [M+H]⁺ 237.9; found 353.1; ¹H NMR (400 MHz, CDCl₃): δ=8.20-8.09 (m, 1H), 7.16-7.11 (m, 1H). The compound structure is confirmed.

Compound int-P-1 (14 g, 58.8 mmol) and methanol (200 ml) were added sequentially in a 500 ml three-necked flask. Under nitrogen protection, it was lowered to 0° C. and sodiummethanol (7.94 g, 147 mmol) was added in batches and reacted at 0° C. for 3 hours. After the reaction was completed, the reaction solution was poured into water (600 ml) and extracted three times with EA (200 ml). The organic phases were combined, washed with saturated brine, dried over anhydrous sodium sulfate, filtered, and concentrated to dryness, and the residue was purified by silica gel chromatographic column with eluent ratio of PE/EtOAc=20/1˜5/1 to obtain the compound int-P-2 (6 g, white solid, purity 99%) in the yield of 40.48%. LCMS (ESI) m/z calcd. for C₇H₅BrFNO₃ [M+H]⁺ 249.9; found 263.9; ¹H NMR (400 MHz, CDCl₃): δ=8.14 (dd, J=9.2, 8.5 Hz, 1H), 6.81 (dd, J=9.4, 1.5 Hz, 1H), 4.04 (s, 3H). The compound structure is confirmed.

To a 250 ml triple-necked flask were sequentially added compound int-P-2 (6 g, 24.0 mmol), EtOH/H₂O=3/1 (120 ml), iron powder (6.70 g, 120 mmol) and ammonium chloride (6.42 g, 120 mmol). The reaction was carried out under nitrogen protection at 80° C. for 4 hours. After the reaction was completed, the reaction solution was reduced to room temperature, filtered, and the filtrate was poured into water (300 ml) and extracted three times with EA (200 ml). The organic phases were combined, washed with saturated brine, dried over anhydrous sodium sulfate, filtered, and concentrated to dryness, and the residue was purified by silica gel chromatographic column with eluent ratio of PE/EtOAc=20/1˜5/1 to obtain the compound int-P-3 (4.7 g, yellow oil, purity 99.619%) in the yield: 88.75%. LCMS (ESI) m/z caled. for C₇H₇BrFNO [M+H]⁺ 219.9; found 219.9; ¹H NMR (400 MHz, DMSO_d₆): δ=6.77-6.70 (m, 2H), 4.91 (s, 2H), 3.73 (s, 3H). The compound structure is confirmed.

To a 250 ml triple-necked vial were sequentially added compound int-P-3 (4.7 g, 21.4 mmol), EtOH (100 ml) and 5-(methoxymethylene)-2,2-dimethyl-1,3-dioxane-4,6-dione (4.78 g, 25.7 mmol). The reaction was refluxed under nitrogen protection for 16 hours. After the reaction was completed, the reaction solution was reduced to room temperature and filtered by extraction to give compound int-P-4 (7.8 g, off-white solid, purity 99.659%), yield: 97.20%. LCMS (ESI) m/z calcd. for C₁₄H₁₃BrFNO₅ [M+H]⁺ 374.0; found 317.8; ¹H NMR (400 MHz, DMSO_d₆): δ=11.25 (d, J=14.3 Hz, 1H), 8.55 (d, J=14.3 Hz, 1H), 7.82 (t, J=9.1 Hz, 1H), 7.05 (d, J=8.3 Hz, 1H), 3.91 (s, 3H), 1.68 (s, 6H). The compound structure is confirmed.

Compound int-P-4 (7.8 g, 20.8 mmol) and diphenyl ether (100 ml) were added sequentially in a 250 ml single-necked vial. The reaction was carried out at 240° C. for 1 h under nitrogen protection. After completion of the reaction, the reaction solution was reduced to room temperature and the compound int-P-5 (3.7 g, brown solid, purity 99.415%) was obtained by filtration, yield: 64.90%. LCMS (ESI) m/z calcd. for C₁₀H₇BrFNO₂ [M+H]⁺ 271.9; found 271.9; ¹H NMR (400 MHz, DMSO_d₆). 6=11.95 (s, 1H), 7.83 (t, J=6.3 Hz, 1H), 7.39 (d, J=7.8 Hz, 1H), 6.09 (d, J=7.2 Hz, 1H), 3.95 (s, 3H). The compound structure is confirmed.

To a 100 ml triple-necked flask was added sequentially compound int-P-5 (3.6 g, 13.2 mmol), toluene (50 ml) and POCl₃ (6.07 g, 39.5 mmol). The reaction was carried out under nitrogen protection at 100° C. for 16 hours. After completion of the reaction, the reaction solution was reduced to room temperature, concentrated and adjusted to weak basicity with saturated NaHCO₃ solution and extracted three times with DCM (100 ml). The organic phases were combined, washed with saturated brine, dried over anhydrous sodium sulfate, filtered, and concentrated to dryness, and the residue was purified by silica gel chromatographic column with the eluent ratio of PE/EtOAc=50/1˜30/1 to obtain the compound int-P-6 (2.7 g, off-white solid, purity 99.539%) in the yield of 70.45%. LCMS (ESI) m/z calcd. for C₁₀H₆BrClFNO [M+H]⁺ 289.9; found 289.9; ¹H NMR (400 MHz, DMSO_d₆): δ=8.73 (d, J=4.7 Hz, 1H), 7.86 (d, J=4.7 Hz, 1H), 7.30 (s, 1H), 4.06 (s, 3H), which shows that the compound is structurally correct.

To a 100 ml triple-necked flask were sequentially added compounds int-P-6 (1.8 g, 6.2 mmol), int-A-2 (1.95 g, 6.2 mmol), cesium carbonate (4.04 g, 12.4 mmol) and DMSO (40 ml). The reaction was carried out under nitrogen protection at 100° C. for 16 hours. After the reaction was completed, the reaction solution was reduced to room temperature, decanted into water (200 ml) and extracted three times with DCM (100 ml). The organic phases were combined, washed with saturated brine, dried over anhydrous sodium sulfate, filtered, and concentrated to dryness. The residue was purified by silica gel column with eluent ratio of PE/EtOAc=5/1˜2/1 to give compound int-P (0.9 g, off-white solid, purity 92.536%), yield: 24.19%. LCMS (ESI) m/z calcd. for C₂₇H₂₀BrF₂N₃O₄ [M+H]⁺ 568.1; found 568.0; ¹H NMR (400 MHz, DMSO_d₆): δ=10.23 (s, 1H), 10.05 (s, 1H), 8.59 (d, J=5.1 Hz, 1H), 7.80 (d, J=8.8 Hz, 2H), 7.65 (dd, J=8.9, 5.1 Hz, 2H), 7.52 (s, 1H), 7.28 (d, J=8.9 Hz, 2H), 7.16 (t, J=8.9 Hz, 2H), 6.66 (d, J=5.1 Hz, 1H), 4.05 (s, 3H), 1.49 (s, 4H). The compound structure is confirmed.

To a 100 ml single-necked vial was sequentially added compound int-P (1.2 g, 2.11 mmol), 4-hydroxymethylpiperidine (486 mg, 4.22 mmol), Pd₂ (dba)₃ (290 mg, 0.32 mmol), BINAP (394 mg, 0.63 mmol), cesium carbonate (1.38 g, 4.22 mmol), and 1,4-dioxane (30 ml). The reaction was carried out under nitrogen protection at 100° C. for 16 hours. After completion of the reaction, the reaction solution was reduced to room temperature, decanted into water (100 ml) and extracted three times with EA (50 ml). The organic phases were combined, washed with saturated brine, dried over anhydrous sodium sulfate, filtered, and concentrated to dryness. The residue was purified by silica gel column with eluent ratio of DCM/MeOH=50/1˜30/1 to afford the compound TPD12139-1 (380 mg, brown solid, purity 91.919%), yield: 27.45%. LCMS (ESI) m/z calcd. for C₃₃H₃₂F₂N₄O₅ [M+H]⁺ 603.2; found 603.1. The compound structure is confirmed.

Compound oxalyl chloride (160 mg, 1.26 mmol) and DCM (4 ml) were sequentially added to a 50 ml triple-necked vial, cooled down to −78° C. A DCM (2 ml) solution of DMSO (197 mg, 2.52 mmol) was added and −78° C. stirred for 0.5 hours. Then DCM (4 ml) solution of compound TPD12139-1 (380 mg, 0.63 mmol) was added and −78° C. stirred for 0.5 hr. Finally, TEA (510 mg, 5.04 mmol) was added and the reaction was slowly returned to room temperature and protected by nitrogen for 1 hour. After completion of the reaction, the reaction solution was poured into saturated NaHCO₃ solution (30 ml) and extracted three times with DCM (10 ml). The organic phases were combined, washed with saturated brine, dried over anhydrous sodium sulfate, filtered, and concentrated to dryness, and the residue was purified by preparative plates with an eluent ratio of DCM/MeOH=12/1 to afford the compound TPD12139-2 (160 mg, yellow solid, purity 92.928%), yield: 39.26%. LCMS (ESI) m/z calcd. for C₃₃H₃₀F₂N₄O₅ [M+H]⁺ 601.2; found 601.1; ¹H NMR (400 MHz, DMSO_d₆): δ=10.20 (s, 1H), 10.05 (s, 1H), 9.68 (s, 1H), 8.50 (d, J=5.1 Hz, 1H), 7.77 (d, J=8.7 Hz, 2H), 7.64 (dd. J=8.7, 5.1 Hz, 2H), 7.33 (s, 1H), 7.23 (d, J=8.8 Hz, 2H), 7.15 (t, J=8.8 Hz, 2H), 6.50 (d, J=5.1 Hz, 1H), 3.96 (s, 3H), 3.37 (br. s, 2H), 3.19 (t, J=11.1 Hz, 2H). 2.58-2.53 (m, 1H), 1.95 (d, J=10.2 Hz, 2H), 1.70-1.60 (m, 2H), 1.48 (s, 4H). The compound structure is confirmed.

To a 25 ml single-necked vial was sequentially added compound TPD12112-3 (60 mg, 0.15 mmol), compound TPD12139-2 (87 mg, 0.15 mmol), sodium triacetoxyborohydride (62 mg, 0.29 mmol) and DCE (5 ml). The reaction was carried out under nitrogen protection at 25° C. for 16 hours. After the reaction was completed, the reaction solution was decanted into water (15 ml) and an appropriate amount of saturated NaHCO₃ solution was added to make the reaction solution alkaline. The organic phase was separated and the aqueous phase was extracted twice with DCM (10 ml). The organic phases were combined, washed with saturated brine, dried over anhydrous sodium sulfate, filtered, and concentrated to dryness. The residue was prepared by high performance liquid chromatography (HPLC) with the following parameters: column: sunfire 5 μm 19-150 mm; mobile phase: acetonitrile-water (0.1% FA); gradient: 5-40/7 min to afford the compound TPD12139 (26.35 mg, yellow solid, purity 96.683%) in the following yield: 16.29%. LCMS (ESI) m/z calcd. for C₅₃H₅₄F₂N₁₀O₈ [M+H]⁺ 997.4; found 499.4; 997.3; ¹H NMR (400 MHz, DMSO_d₆): δ=11.17 (s, 1H), 11.01 (s, 1H), 10.21 (s, 1H), 10.06 (s, 1H), 8.68 (s, 1H), 8.49 (d, J=5.1 Hz, 1H), 8.36 (s, 1H), 8.23 (s, 1.7H), 7.77 (d, J=8.8 Hz, 2H), 7.64 (dd, J=8.9, 5.1 Hz, 2H), 7.32 (s, 1H), 7.23 (d, J=8.9 Hz, 2H), 7.15 (t, J=8.9 Hz, 2H). 6.49 (d, J=5.1 Hz, 1H), 5.09 (dd, J=13.2, 5.0 Hz, 1H), 4.53 (d, J=18.4 Hz, 1H), 4.39 (d, J=18.3 Hz, 2H), 3.95 (s, 3H), 3.67 (s, 2H), 3.31 (d, J=10.9 Hz, 2H), 3.13-2.85 (m, 10H), 2.67-2.54 (m, 4H), 2.41-2.33 (m, 2H), 1.99-1.91 (m, 2H), 1.75 (d, J=10.9 Hz, 2H), 1.55 (br. s, 1H), 1.48 (s, 4H), 1.32 (d, J=11.0 Hz, 2H). The compound structure is confirmed.

### Example 79: Synthesis of Compound TPD12140

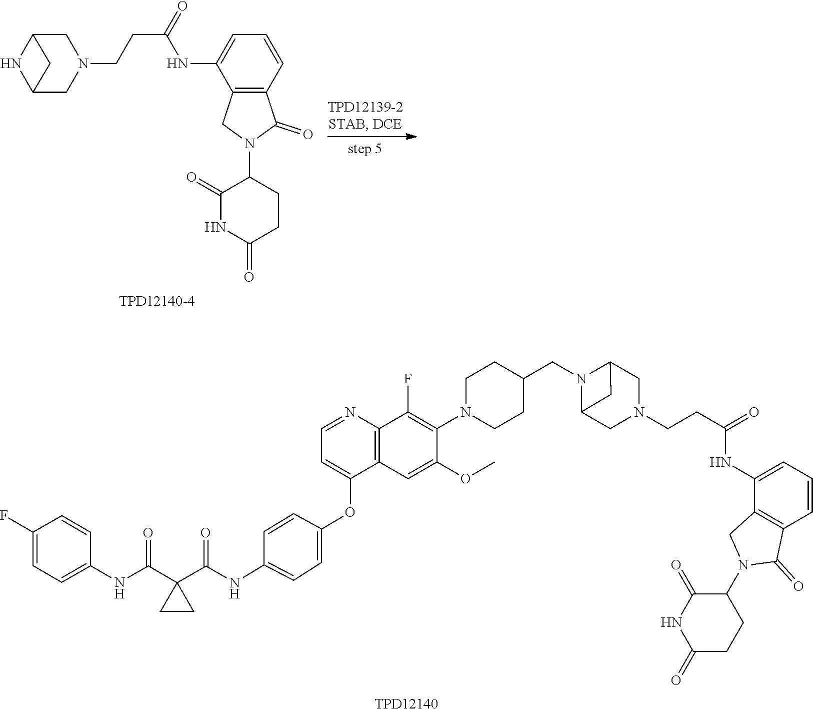 [SMILES: O=C1CCC(N2Cc3c(NC(=O)CCN4CC5CC(C4)N5)cccc3C2=O)C(=O)N1 ; COc1cc2c(Oc3ccc(NC(=O)C4(C(=O)Nc5ccc(F)cc5)CC4)cc3)ccnc2c(F)c1N1CCC(CN2C3CC2CN(CCC(=O)Nc2cccc4c2CN(C2CCC(=O)NC2=O)C4=O)C3)CC1]

To a 25 ml single-necked vial was sequentially added compound TPD12140-4 (60 mg, 0.146 mmol), compound TPD12139-2 (88 mg, 0.146 mmol), sodium triacetoxyborohydride (62 mg, 0.292 mmol) and DCE (5 ml). The reaction was carried out for 16 hours at 25° C. under nitrogen protection. After the reaction was completed, the reaction solution was decanted into water (15 ml) and appropriate amount of saturated NaHCO₃ solution was added to make the reaction solution alkaline. The organic phase was separated and the aqueous phase was extracted twice with DCM (10 ml). The organic phases were combined, washed with saturated brine, dried over anhydrous sodium sulfate, filtered, and concentrated to dryness. The residue was prepared by high performance liquid chromatography (HPLC) with the following parameters: column: T3 5 μm 19-150 mm; mobile phase: acetonitrile-water (0.1% FA); gradient: 20-35/7 min to afford the compound TPD12140 (21.1 mg, yellow solid, purity 98.864%) in the following yield: 13.31%. LCMS (ESI) m/z calcd. for C₅₄H₅₅F₂N₉O₈ [M+H]⁺ 996.4; found 498.8; 996.3; 1H NMR (400 MHz, DMSO_d₆): δ=11.05 (s, 1H), 10.21 (s, 1H), 10.06 (s, 1H), 9.97 (s, 1H), 8.49 (d, J=5.1 Hz, 1H), 7.81-7.76 (m, 3H), 7.64 (dd, J=8.9, 5.1 Hz, 2H), 7.54-7.48 (m, 2H), 7.32 (s, 1H), 7.22 (d, J=8.9 Hz, 2H), 7.15 (t, J=8.9 Hz, 2H), 6.49 (d, J=5.1 Hz 1H), 5.16 (dd, J=13.3, 5.1 Hz, 1H), 4.40 (d, J=17.5 Hz, 1H), 4.32 (d, J=17.4 Hz, 1H), 3.95 (s, 3H), 3.67 (s, 2H), 3.31 (d, J=11.4 Hz, 2H), 3.13-2.88 (m, 10H). 2.67-2.54 (m, 3H), 2.33-2.28 (m, 3H), 2.05-2.02 (m, 1H), 1.84 (d, J=7.3 Hz, 1H), 1.75 (d, J=11.4 Hz, 2H), 1.56 (s, 1H), 1.48 (s, 4H), 1.37-1.32 (m, 2H). The compound structure is confirmed.

### Example 80: Synthesis of Compound TPD12148

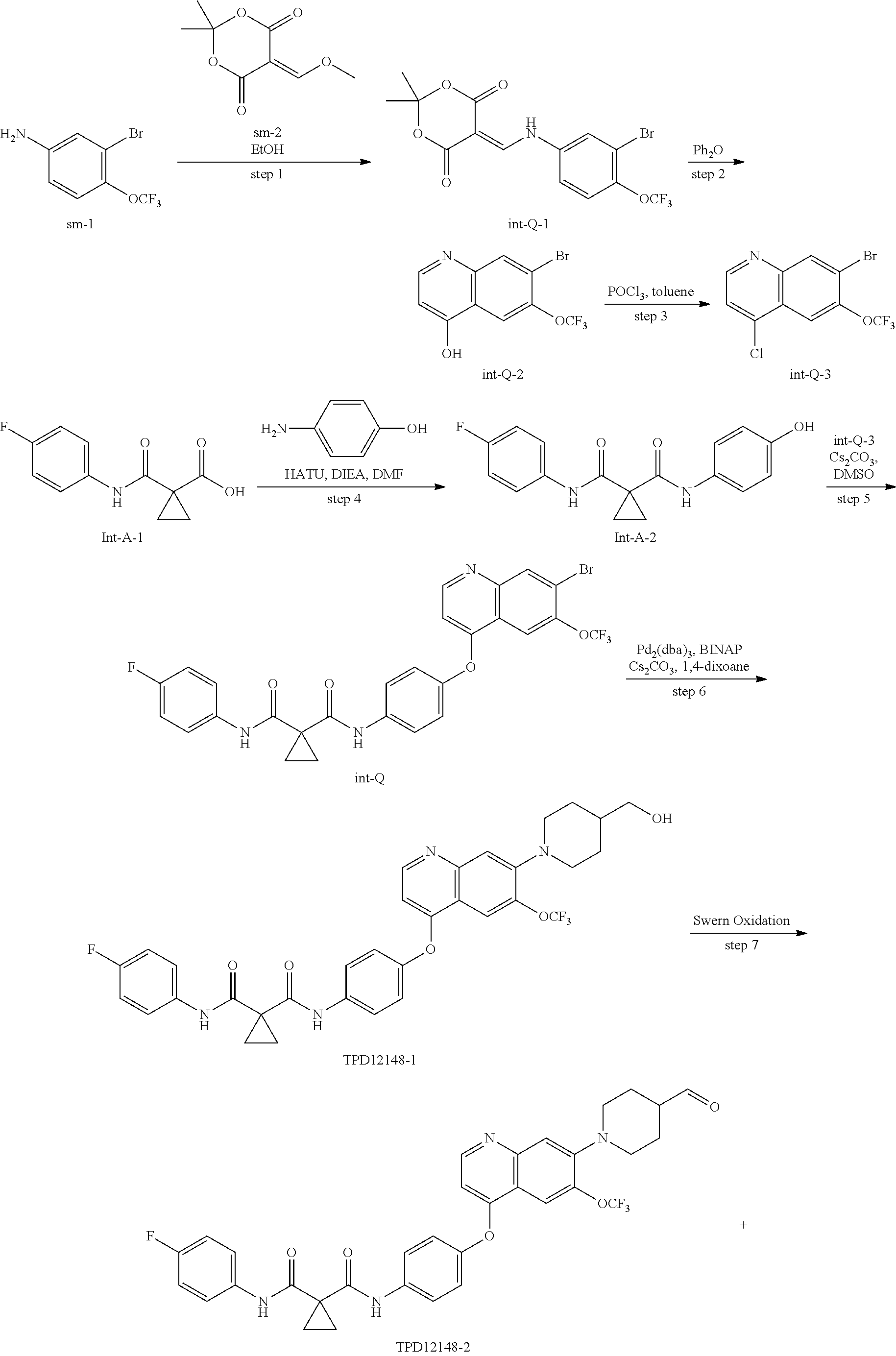

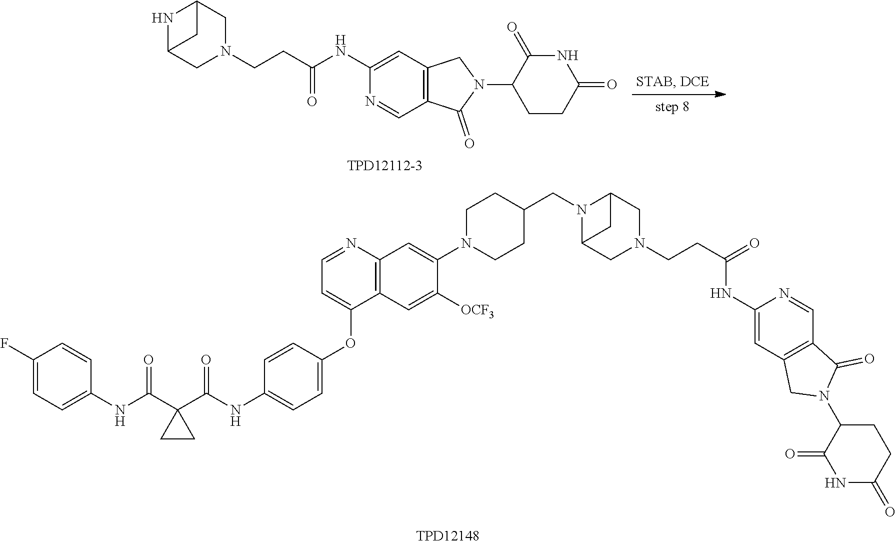 [SMILES: O=C1CCC(N2Cc3cc(NC(=O)CCN4CC5CC(C4)N5)ncc3C2=O)C(=O)N1 ; O=C1CCC(N2Cc3cc(NC(=O)CCN4CC5CC(C4)N5CC4CCN(c5cc6nccc(Oc7ccc(NC(=O)C8(C(=O)Nc9ccc(F)cc9)CC8)cc7)c6cc5OC(F)(F)F)CC4)ncc3C2=O)C(=O)N1]

Compound sm-1 (25 g, 0.0976 mol), EtOH (300 ml) and sm-2 (18.17 g, 0.0976 mol) were added sequentially in a 500 ml triple-necked vial. The reaction was refluxed under nitrogen protection for 16 hours. After completion of the reaction, the reaction solution was reduced to room temperature and filtered by extraction to afford the compound int-Q-1 (35 g, yellow solid, purity 98.883%), yield: 86.48%. LCMS (ESI) m/z calcd. for C₁₄H₁₁BrF₃NO₅ [M+H]⁺ 409.98, 411.98; found 351.8, 353.8; ¹H NMR (400 MHz, DMSO_d₆): δ=11.26 (s, 1H), 8.58 (s, 1H), 8.16 (d, J=2.7 Hz, 1H), 7.72 (dd, J=9.0, 2.7 Hz, 1H), 7.63-7.52 (m, 1H), 1.68 (s, 6H). The compound structure is confirmed.

Compound int-Q-1 (12 g, 0.0292 mol) and diphenyl ether (120 ml) were added sequentially in a 250 ml single-necked vial. The reaction was carried out at 240° C. for 30 min under nitrogen protection. The other two batches were operated identically. After completion of the reaction, the reaction solution was reduced to room temperature and the compound int-Q-2 (17 g, white solid, purity 93.766%) was obtained by filtration, yield: 60.61%. LCMS (ESI) m/z calcd. for C₁₀H₅BrF₃NO₂ [M+H]⁺ 307.95, 309.95; found 307.9, 309.9; ¹H NMR (400 MHz, DMSO_d₆): δ 11.99 (s, 1H), 8.05-8.00 (m, 1H), 8.00-7.88 (m, 1H), 7.70 (dd, J=35.0, 9.1 Hz, 1H), 6.11 (dd, J=14.7, 7.4 Hz, 1H). The compound structure is confirmed.

To a 500 ml triple-necked flask was sequentially added compound int-Q-2 (17 g, 0.0551 mol), toluene (200 ml) and POCl₃ (25.37 g, 0.165 mol). The reaction was carried out under nitrogen protection at 100° C. for 16 hours. After completion of the reaction, the reaction solution was reduced to room temperature, concentrated and adjusted to alkaline with saturated NaHCO₃ solution and extracted three times with DCM (200 ml). The organic phases were combined, washed with saturated brine, dried over anhydrous sodium sulfate, filtered, and concentrated to dryness, and the residue was purified by silica gel chromatographic column with eluent ratio of PE/EtOAc=50/1˜30/1 to obtain the compound int-Q-3 (4.2 g, white solid, purity 99.055%) in the yield: 25.49%. LCMS (ESI) m/z calcd. for C₁₀H₄BrClF₃NO [M+H]⁺ 325.91, 327.91; found 325.8, 327.8; ¹H NMR (400 MHz, DMSO_d₆): δ=8.95 (d, J=4.8 Hz, 1H), 8.61 (s, 1H), 8.16 (d, J=1.0 Hz, 1H), 7.93 (d, J=4.7 Hz, 1H). The compound structure is confirmed.

Compound int-A-1 (34 g, 0.152 mol), 4-aminophenol (23.27 g, 0.213 mol), HATU (81.07 g, 0.213 mol), DIEA (49.21 g, 0.381 mol), and DMF (300 ml) were added sequentially in a 1000 ml single-necked vial. The reaction was carried out under nitrogen protection at 25° C. for 16 hours. After the reaction was completed, the reaction solution was concentrated to dryness. The residue was purified by silica gel column with eluent ratio of DCM/MeOH=100/1˜30/1 to afford compound int-A-2 (22 g, brown oil, purity 94.060%), yield: 43.20%. LCMS (ESI) m/z calcd. for C₁₇H₁₅FN₂O₃ [M+H]⁺ 315.11; found 315.0; ¹H NMR (400 MHz, DMSO_d₆): δ=10.18 (s, 1H), 9.75 (s, 1H), 9.28 (s, 1H), 7.72-7.54 (m, 2H), 7.35 (d, J=8.7 Hz, 2H), 7.14 (t, J=8.9 Hz, 2H), 6.70 (d, J=8.8 Hz, 2H). 1.45 (s, 4H). The compound structure is confirmed.

Compounds int-Q-3 (4.2 g, 0.0129 mol), int-A-2 (4.87 g, 0.0154 mol), cesium carbonate (8.41 g, 0.0258 mol) and DMSO (50 ml) were added sequentially to a 100 ml triple-necked vial. The reaction was carried out under nitrogen protection at 120° C. for 16 hours. After the reaction was completed, the reaction solution was reduced to room temperature, decanted into water (300 ml) and extracted three times with DCM (200 ml). The organic phases were combined, washed with saturated brine, dried over anhydrous sodium sulfate, filtered, and concentrated to dryness. The residue was purified by silica gel column with eluent ratio of PE/EtOAc=5/1˜2/1 to give compound int-Q (7 g, yellow solid, purity 96.576%), yield: 86.82%. LCMS (ESI) m/z calcd. for C₂₇H₁₈BrF₄N₃O₄ [M+H]⁺ 604.04, 606.04; found 603.9, 605.9; ¹H NMR (400 MHz, DMSO_d₆): δ=10.23 (s, 1H), 10.05 (s, 1H), 8.77 (d, J=5.2 Hz, 1H), 8.53 (s, 1H), 8.31 (s, 1H), 7.80 (d, J=8.8 Hz, 2H), 7.65 (dd, J=8.7, 5.1 Hz, 2H), 7.31 (d, J=8.8 Hz, 2H), 7.15 (t, J=8.8 Hz, 2H), 6.69 (d, J=5.2 Hz, 1H), 1.48 (s, 4H). The compound structure is confirmed.

To a 100 ml single-necked vial were sequentially added compound int-Q (3.5 g, 0.0058 mol), 4-hydroxymethylpiperidine (1.34 g, 0.0116 mol), Pd₂ (dba)₃ (0.53 g, 0.0005 mol), BINAP (0.72 g, 0.0011 mol), cesium carbonate (3.78 g, 0.0116 mol) and 1,4-dioxane (70 ml). The reaction was carried out under nitrogen protection at 100° C. for 16 hours. After completion of the reaction, the reaction solution was reduced to room temperature, decanted into water (200 ml) and extracted three times with EA (150 ml). The organic phases were combined, washed with saturated brine, dried over anhydrous sodium sulfate, filtered, and concentrated to dryness. The residue was purified by silica gel column with eluent ratio of DCM/MeOH=80/1˜30/1 to afford the compound TPD12148-1 (2 g, yellow solid, purity 68.172%), yield: 36.21%. LCMS (ESI) m/z calcd. for C₃₃H₃₀F₄N₄O₅ [M+H]639.22; found 639.0; ¹H NMR (400 MHz, DMSO_d₆): δ=10.21 (s, 1H), 10.06 (s, 1H), 8.63 (d, J=5.2 Hz, 1H), 8.04 (d, J=1.0 Hz, 1H), 7.77 (d, J=8.9 Hz, 2H), 7.65 (dd, J=9.0, 5.1 Hz, 2H). 7.54 (s, 1H), 7.26 (d, J=9.0 Hz, 2H), 7.16 (t, J=8.9 Hz, 2H), 6.47 (d, J=5.2 Hz, 1H), 4.55 (t, J=5.3 Hz, 1H), 3.52 (d, J=11.8 Hz, 2H), 3.33-3.28 (m, 2H), 2.77 (t J=11.2 Hz, 2H), 1.83 (d, J=11.5 Hz, 2H), 1.58 (s, 1H), 1.48 (s, 4H), 1.34 (dd, J=20.7, 11.7 Hz, 2H). The compound structure is confirmed.

Compound oxalyl chloride (0.41 g, 0.0032 mol) and DCM (15 ml) were sequentially added to a 50 ml triple-necked vial, cooled down to −78° C. A DCM (5 ml) solution of DMSO (0.5 g, 0.0064 mol) was added and stirred for 0.5 hr at −78° C. The mixture was then stirred for 0.5 hr. Then DCM (10 ml) solution of compound TPD12148-1 (1 g, 0.0016 mol) was added and −78° C. stirred for 0.5 hours. Finally, TEA (1.3 g, 0.0128 mol) was added and the reaction was slowly restored to room temperature and protected by nitrogen for 1 hour. After completion of the reaction, the reaction solution was poured into saturated NaHCO₃ solution (60 ml) and extracted three times with DCM (30 ml). The organic phases were combined, washed with saturated brine, dried over anhydrous sodium sulfate, filtered, and concentrated to dryness, and the residue was purified by silica gel chromatographic column with eluent ratio of DCM/MeOH=80/1˜30/1 to afford the compound TPD12148-2 (0.7 g, yellow solid, purity 70.18%) in 50.00% yield. LCMS (ESI) m/z calcd. for C₃₃H₂₈F₄N₄O₅ [M+H]⁺ 637.2; found 637.1; ¹H NMR (400 MHz, DMSO_d₆): δ=10.21 (s, 1H), 10.06 (s, 1H), 8.64 (t, J=5.0 Hz, 1H), 8.05 (d, J=7.6 Hz, 1H), 7.77 (d, J=8.7 Hz, 2H), 7.64 (dd, J=8.9, 5.1 Hz, 2H), 7.54 (d, J=11.2 Hz, 1H), 7.26 (d, J=8.8 Hz, 2H), 7.15 (t, J=8.9 Hz, 2H), 6.48 (t, J=5.6 Hz, 1H), 3.57-3.45 (m, 3H). 3.26 (s, 2H), 3.04-2.82 (m, 1H), 2.72 (t, J=12.1 Hz, 1H), 2.06 (dd, J=19.6, 11.2 Hz, 1H), 1.85 (d, J=12.3 Hz, 1H), 1.68 (dd, J=20.3, 10.4 Hz, 1H), 1.48 (s, 4H). The compound structure is confirmed.

To a 25 ml single-necked vial was sequentially added compound TPD12112-3 (150 mg, 0.36 mmol), compound TPD12148-2 (231 mg, 0.36 mmol), sodium triacetoxyborohydride (154 mg, 0.72 mmol) and DCE (5 ml). The reaction was carried out under nitrogen protection at 25° C. for 16 hours. After the reaction was completed, the reaction solution was decanted into water (15 ml) and an appropriate amount of saturated NaHCO₃ solution was added to make the reaction solution alkaline. The organic phase was separated and the aqueous phase was extracted twice with DCM (10 ml). The organic phases were combined, washed with saturated brine, dried over anhydrous sodium sulfate, filtered, and concentrated to dryness. The residue was prepared by high performance liquid chromatography (HPLC) with the following parameters: column: sunfire 5 μm 19-150 mm; mobile phase: acetonitrile-water (0.1% FA); gradient: 5-40/7 min to give compound TPD12148 (40.0 mg, yellow solid, purity 98.899%), yield: 9.98%. LCMS (ESI) m/z calcd. for C₅₃H₅₂F₄N₁₀O₈ [M+H]⁺ 1033.4; found 517.3; 1033.3; ¹H NMR (400 MHz, DMSO_d₆): δ=11.18 (s, 1H), 11.03 (s, 1H), 10.21 (s, 1H), 10.06 (s, 1H), 8.68 (s, 1H), 8.63 (d, J=5.2 Hz, 1H), 8.36 (s, 1H), 8.21 (s, 1.2H), 8.04 (s, 1H), 7.77 (d, J=8.8 Hz, 2H), 7.64 (dd, J=8.9, 5.1 Hz, 2H), 7.53 (s, 1H),

7.26 (d, J=8.9 Hz, 2H), 7.15 (t, J=8.9 Hz, 2H), 6.47 (d, J=5.2 Hz, 1H), 5.10 (dd, J=13.3, 5.0 Hz, 1H), 4.54 (d, J=18.5 Hz, 1H), 4.39 (d, J=18.4 Hz, 1H), 3.60 (s, 2H), 3.49 (d, J=11.2 Hz, 2H), 3.02-2.86 (m, 7H), 2.74 (t, J=11.2 Hz, 2H), 2.66-2.61 (m, 3H), 2.41-2.34 (m, 4H), 2.00-1.97 (m, 1H), 1.92-1.85 (m, 3H), 1.53 (s, 1H), 1.48 (s, 4H), 1.32 (dd, J=22.0, 11.1 Hz, 2H). The compound structure is confirmed.

### Example 81: Synthesis of Compound TPD12149

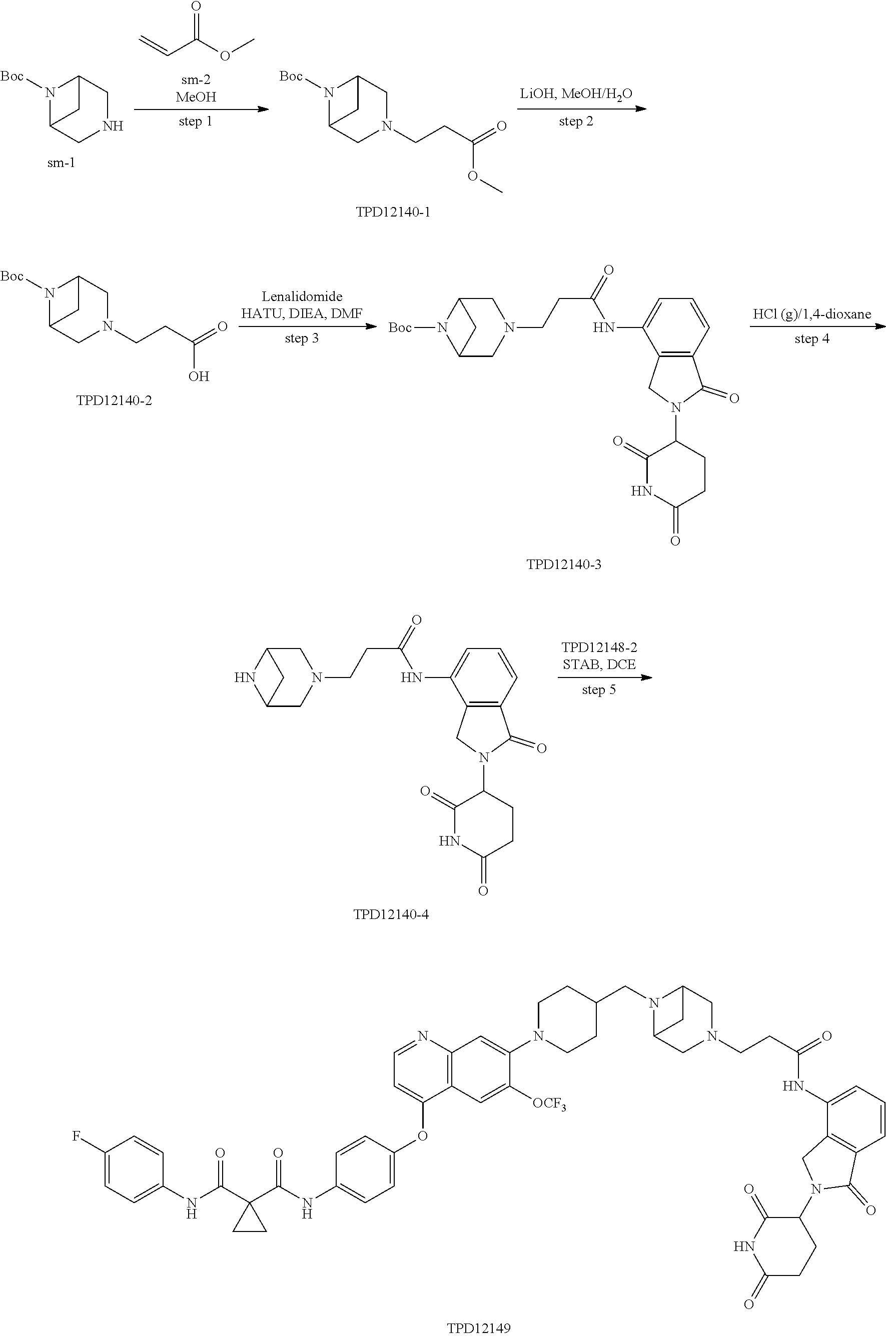

To a 100 ml single-necked vial were sequentially added compound sm-1 (2 g, 0.01 mol), methyl acrylate (1.29 g, 0.015 mol) and MeOH (30 ml). The reaction was carried out under nitrogen protection at 25° C. for 16 hours. After completion of the reaction, the reaction solution was concentrated to dryness to give the compound TPD12140-1 (2.9 g, colorless oil, purity 92.339%), yield: 81%. LCMS (ESI) m/z calcd. for C₁₄H₂₄N₂O₄ [M+H]⁺ 285.17; found 285.1; ¹H NMR (400 MHz, DMSO_d₆): δ=3.93 (d, J=5.8 Hz, 2H), 3.60 (d, J=10.8 Hz, 3H), 3.05-2.86 (m, 2H), 2.80-2.63 (m, 4H), 2.46 (t, J=7.1 Hz, 2H), 2.30-2.21 (m, 1H), 1.49 (d, J=7.9 Hz, 1H), 1.38 (s, 9H). The compound structure is confirmed.

To a 100 ml single-necked vial were sequentially added compound TPD12140-1 (2.9 g, 0.0102 mol), lithium hydroxide monohydrate (1.28 g, 0.0306 mol), MeOH (20 ml) and H₂O (10 ml). The reaction was carried out for 16 hours at 25° C. under hydrogen protection. After completion of the reaction, the reaction solution was reduced to room temperature, filtered and concentrated to dryness to afford the compound TPD 12140-2 (1 g, yellow oil, purity 70.663%), yield: 25.49%. LCMS (ESI) m/z calcd. for C₁₃H₂₂N₂O₄ [M+H]⁺ 271.16; found 271.1; ¹H NMR (400 MHz. DMSO_d₆): δ=3.95-3.89 (m, 3H), 3.03-2.82 (m, 3H), 2.70-2.63 (m, 4H), 2.19 (dd, J=16.0, 8.3 Hz, 3H), 1.38 (s, 9H). The compound structure is confirmed.

Compound TPD12140-2 (1020 mg, 3.76 mmol), lenalidomide (650 mg, 2.51 mmol), DIEA (648 mg, 5.01 mmol), HATU (1097 mg, 5.01 mmol), and DMF (25 ml) were added sequentially to a 25 ml single mouth vial. The reaction was carried out under nitrogen protection at 50° C. for 16 hours. After completion of the reaction, the reaction solution was brought to room temperature, decanted into water (100 ml) and extracted three times with EA (90 ml). The organic phases were combined, washed with saturated brine, dried over anhydrous sodium sulfate, filtered, and concentrated to dryness. The residue was purified by column chromatography with eluent ratio of DCM/MeOH=100/1˜20/1 to give compound TPD12140-3 (770 mg, red solid, purity 77.576%), yield: 46.48%. LCMS (ESI) m/z calcd. for C₂₆H₃₃N₅O [M+H]⁺ 512.24; found 512.3; ¹H NMR (400 MHz, DMSO_d₆): δ=11.03 (s, 1H), 9.88 (s, 1H), 7.81-7.76 (m, 1H), 7.54-7.47 (m, 2H), 5.19-5.11 (m, J=13.3, 5.1 Hz, 1H), 4.41-4.29 (m, 2H). 3.99 (t, J=17.3 Hz, 3H), 3.01-2.75 (m, 8H), 2.35-2.23 (m, 2H), 1.43-1.29 (m, 12H). The compound structure is confirmed.

Compound TPD12140-3 (110 mg, 0.21 mmol) 4N HCl/1,4-dioxane (12 ml) was added sequentially in a 25 ml single-necked vial. The reaction was carried out at 25° C. for 2 h. After completion of the reaction, it was concentrated to dryness to give the crude compound TPD12140-4 (110 mg, yellow solid), yield: 99.44%. LCMS (ESI) m/z calcd. for C₂₁H₂₅N₅O₄ [M+H]⁺ 412.19; found 412.0. The compound structure is confirmed.

To a 25 ml single-necked vial were sequentially added compound TPD12140-4 (110 mg, 0.27 mmol), compound TPD12148-2 (171 mg, 0.27 mmol), sodium triacetoxyborohydride (113 mg, 0.53 mmol) and DCE (2 ml). The reaction was carried out under nitrogen protection at 25° C. for 16 hours. After the reaction was completed, the reaction solution was poured into water (5 ml) and an appropriate amount of saturated NaHCO₃ solution was added to make the reaction solution alkaline. It was extracted three times with DCM (15 ml). The organic phases were combined, washed with saturated brine, dried over anhydrous sodium sulfate, filtered, and concentrated to dryness. The residue was prepared by high performance liquid chromatography (HPLC) with the following parameters: column: XBridge-1 5 μm 19-150 mm; mobile phase: acetonitrile-water (0.1% FA); gradient: 10-55/8 min to afford the compound TPD12149 (31.5 mg, yellow solid, purity 98.694%) in the following yield: 11.45%. LCMS (ESI) m/z calcd. for C₅₄H₅₃F₄N₉O₈ [M+H]⁺ 1032.07; found 1032.3; ¹H NMR (400 MHz, DMSO_d₆): δ=11.05 (s, 1H), 10.22 (s, 1H), 10.06 (s, 1H), 9.97 (s, 1H), 8.63 (d, J=5.2 Hz, 1H), 8.23 (s, 2H), 8.04 (s, 1H), 7.78 (t, J=8.3 Hz, 3H), 7.70-7.58 (m, 2H), 7.58-7.40 (m, 3H), 7.26 (d, J=8.9 Hz, 2H), 7.15 (t, J=8.9 Hz, 2H), 6.47 (d, J=5.2 Hz, 1H), 5.20-5.12 (m, 1H), 4.40-4.31 (m, J=12.9 Hz, 2H), 3.59 (s, 2H), 3.49 (d, J=10.9 Hz, 2H), 3.02-2.88 (m, 6H), 2.75 (t, J=11.1 Hz, 2H), 2.68-2.54 (m, 3H), 2.42-2.25 (m, 4H), 2.07-2.00 (m, J=5.1 Hz, 1H), 1.90-1.75 (m, 3H), 1.47 (s, 4H), 1.38-1.16 (m, 4H). The compound structure is confirmed.

### Example 82: Synthesis of Compound TPD12150

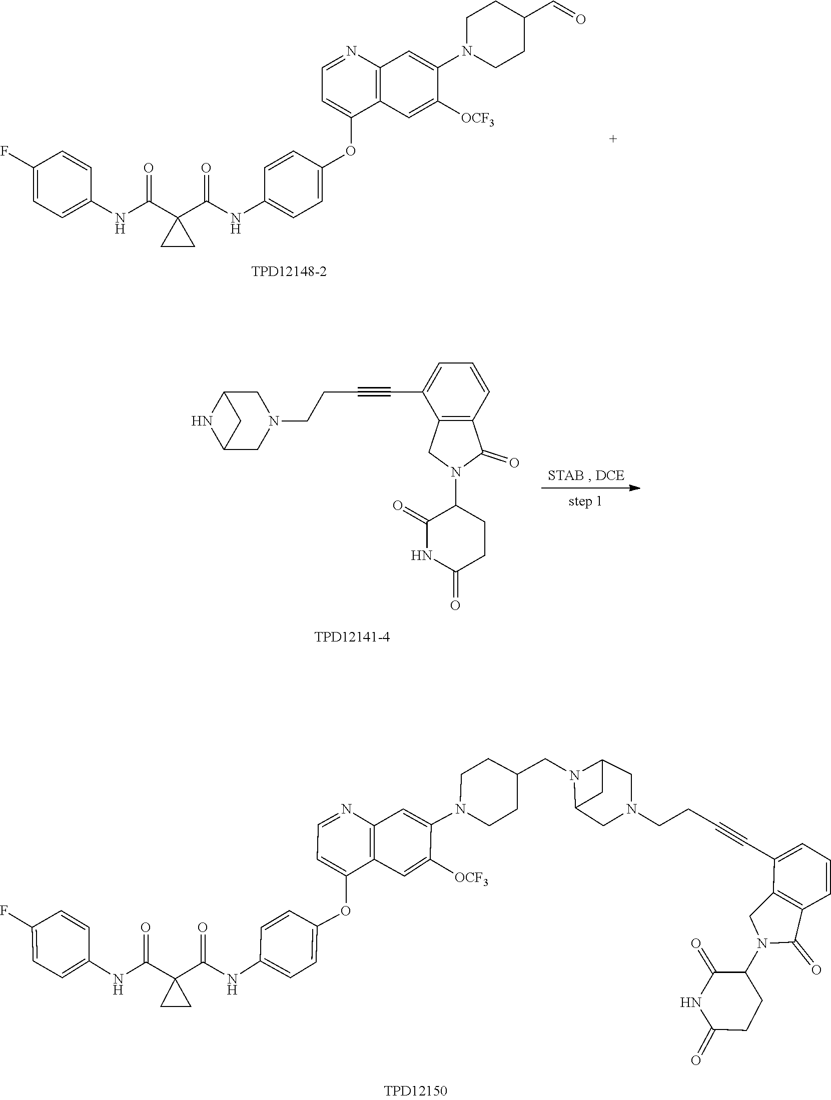 [SMILES: O=CC1CCN(c2cc3nccc(Oc4ccc(NC(=O)C5(C(=O)Nc6ccc(F)cc6)CC5)cc4)c3cc2OC(F)(F)F)CC1 ; O=C1CCC(N2Cc3c(C#CCCN4CC5CC(C4)N5)cccc3C2=O)C(=O)N1 ; O=C1CCC(N2Cc3c(C#CCCN4CC5CC(C4)N5CC4CCN(c5cc6nccc(Oc7ccc(NC(=O)C8(C(=O)Nc9ccc(F)cc9)CC8)cc7)c6cc5OC(F)(F)F)CC4)cccc3C2=O)C(=O)N1]

To a 25 ml single-necked vial were sequentially added compound TPD12148-2 (160 mg, 0.25 mmol), compound TPD12141-4 (99 mg, 0.25 mmol), sodium triacetoxyborohydride (107 mg, 0.50 mmol) and DCE (3 ml). The reaction was carried out under nitrogen protection at 25° C. for 16 hours. After the reaction was completed, the reaction solution was poured into water (10 ml) and an appropriate amount of saturated NaHCO₃ solution was added to make the reaction solution alkaline. It was extracted three times with DCM (5 ml). The organic phases were combined, washed with saturated brine, dried over anhydrous sodium sulfate, filtered, and concentrated to dryness. The residue was prepared by high performance liquid chromatography (HPLC) with the following parameters: column: XBridge-1 5 μm 19-150 mm; mobile phase: acetonitrile-water (0.1% FA); gradient: 10-55/8 min to afford the compound TPD12150 (60.7 mg, yellow solid, purity 99.902%) in the following yield: 22.09%. LCMS (ESI) m/z calcd. for C₅₅H₅₂F₄N₈O₇ [M+H]⁺ 1013.39; found 1013.4; ¹H NMR (400 MHz, DMSO_d₆): δ=11.04 (s, 1H), 10.21 (s, 1H), 10.06 (s, 1H), 8.63 (d, J=5.2 Hz, 1H), 8.04 (s, 1H)), 7.77 (d, J=8.9 Hz, 2H), 7.72 (d, J=7.3 Hz, 1H), 7.67-7.62 (m, 3H), 7.55-7.50 (m, 2H), 7.26 (d, J=8.9 Hz, 2H), 7.15 (t, J=8.9 Hz, 2H), 6.47 (d, J=5.2 Hz, 1H). 5.17 (dd, J=13.3, 5.1 Hz, 1H), 4.46 (d, J=17.7 Hz, 1H), 4.31 (d, J=17.7 Hz, 1H), 3.61 (s, 2H), 3.48 (d, J=10.4 Hz, 3H), 3.07 (d, J=10.9 Hz, 2H), 2.98-2.88 (m, 4H), 2.80-2.65 (m, 4H), 2.60 (d, J=17.2 Hz, 1H), 2.45-2.30 (m, 4H), 2.06-1.99 (m, 1H), 1.85 (br. s, 3H), 1.54 (br. s, 1H), 1.47 (s, 4H), 1.37-1.28 (m, 2H). The compound structure is confirmed.

### Example 83: Synthesis of Compound TPD12162

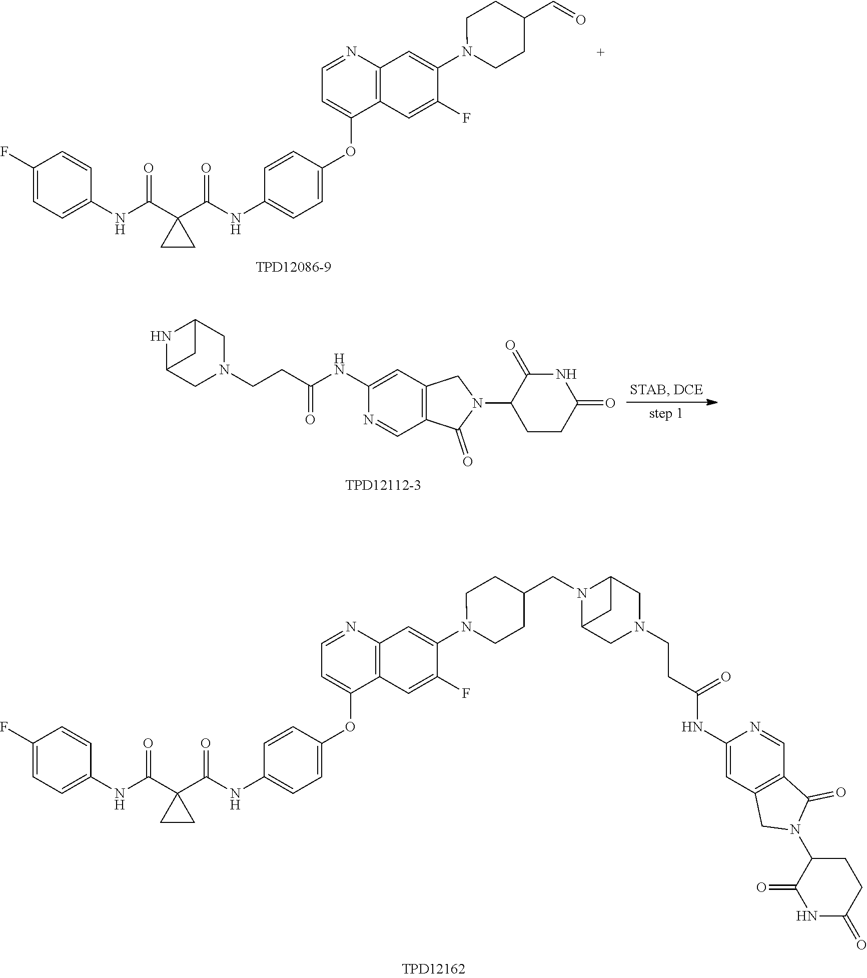

To a 25 ml single-necked vial was sequentially added compound TPD12112-3 (150 mg, 0.36 mmol), compound TPD12086-9 (208 mg, 0.36 mmol), sodium triacetoxyborohydride (154 mg, 0.73 mmol) and DCE (5 ml). The reaction was carried out under nitrogen protection at 25° C. for 16 hours. After the reaction was completed, the reaction solution was decanted into water (15 ml) and an appropriate amount of saturated NaHCO₃ solution was added to make the reaction solution alkaline. The organic phase was separated and the aqueous phase was extracted twice with DCM (10 ml). The organic phases were combined, washed with saturated brine, dried over anhydrous sodium sulfate, filtered, and concentrated to dryness. The residue was prepared by high performance liquid chromatography (HPLC) with the following parameters: column: sunfire 5 μm 19-150 mm; mobile phase: acetonitrile-water (0.1% FA); gradient: 5-40/7 min to afford the compound TPD12162 (52.9 mg, yellow solid, purity 99.423%) in the following yield: 14.96%. LCMS (ESI) m/z calcd. for C52H₅₂F₂N₁₀O₇ [M+H]⁺ 967.4; found 484.4; 967.3; ¹H NMR (400 MHz, DMSO_d₆): δ=11.15 (s, 1H), 11.04 (s, 1H), 10.21 (s, 1H), 10.06 (s, 1H), 8.68 (s, 1H), 8.56 (d J=5.2 Hz, 1H), 8.36 (s, 1H), 8.21 (s, 2.3H), 7.86 (d, J=13.4 Hz, 1H), 7.76 (d, J=8.9 Hz, 2H), 7.64 (dd, J=9.0, 5.1 Hz, 2H), 7.44 (d, J=8.4 Hz, 1H), 7.22 (d, J=8.9 Hz, 2H), 7.15 (t, J=8.9 Hz, 2H), 6.45 (d, J=5.2 Hz, 1H), 5.10 (dd, J=13.3, 5.0 Hz, 1H), 4.53 (d, J=18.5 Hz, 1H), 4.39 (d, J=18.4 Hz, 1H), 3.73 (s, 2H), 3.55 (d J=11.3 Hz, 2H), 3.06-2.86 (m, 8H), 2.75 (t, J=11.3 Hz, 2H), 2.67-2.54 (m, 4H), 2.41-2.30 (m, 2H), 1.99-1.92 (m, 2H), 1.84 (d, J=11.3 Hz, 2H), 1.59 (s, 1H). 1.47 (s, 4H), 1.42-1.37 (m, 2H). The compound structure is confirmed.

### Example 84: Synthesis of Compound TPD12163

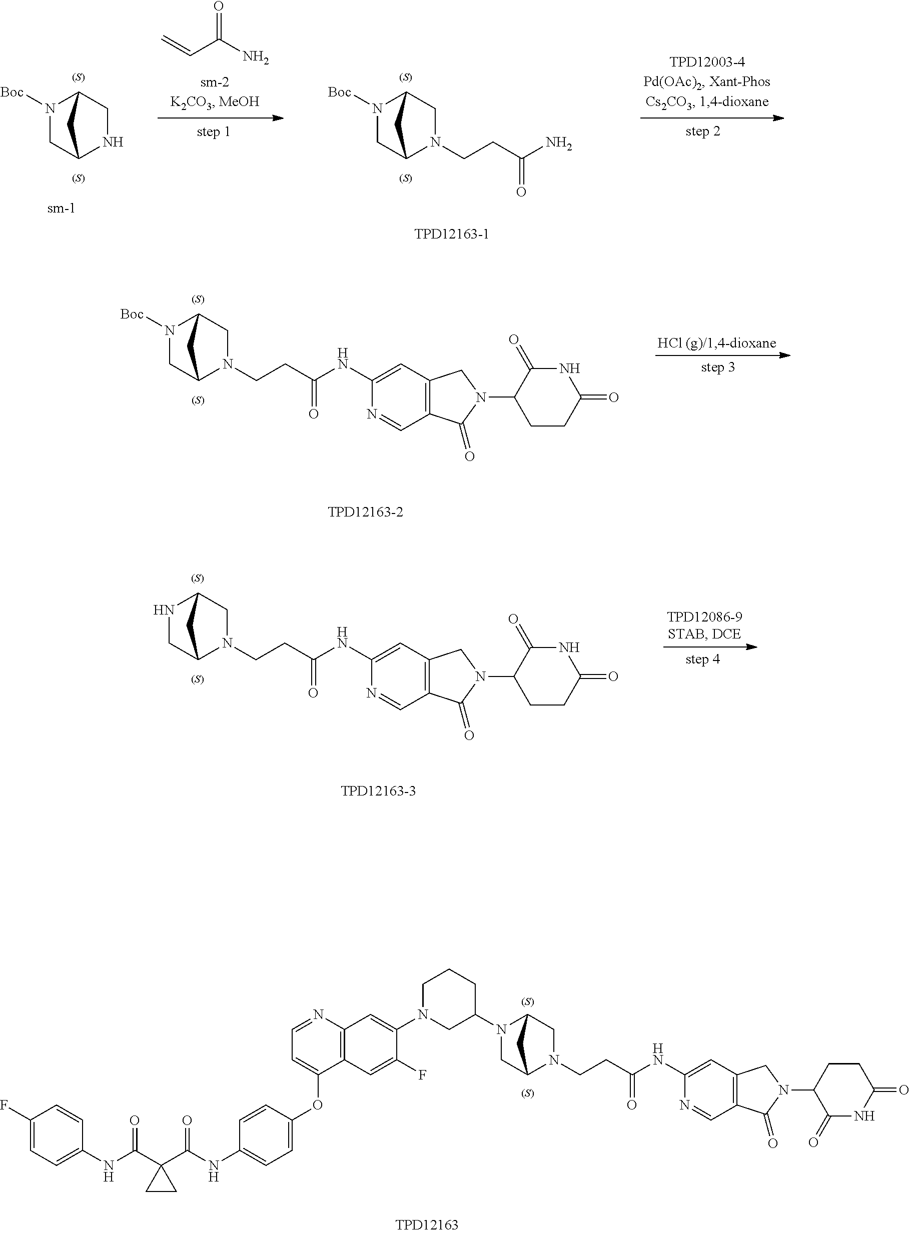

To a 100 ml single-necked vial were sequentially added compound sm-1 (1 g, 0.005 mol), acrylamide (0.36 g, 0.005 mol), K₂CO₃ (1.04 g, 0.0075 mol), and MeOH (10 ml). The reaction was carried out under nitrogen protection at 25° C. for 16 hours. After the reaction was completed, the reaction solution was brought to room temperature, decanted into water (30 ml) and extracted three times with EA (15 ml). The organic phases were combined, washed with saturated brine, dried over anhydrous sodium sulfate, filtered, and concentrated to dryness. The residue was purified by silica gel column with eluent ratio of DCM/MeOH=80/1˜30/1 to obtain the compound TPD12163-1 (0.8 g, white solid, purity 73.888%), yield: 44.00%. LCMS (ESI) m/z calcd. for C₁₃H₂₃N₃O₃ [M+H]⁺ 270.17; found 270.1. The compound structure is confirmed.

To a 25 ml single-necked vial was sequentially added compound TPD12163-1 (800 mg, 2.73 mmol), compound TPD12003-4 (1241 mg, 4.44 mmol), XantPhos (342 mg, 0.59 mmol), Pd(OAc)₂ (133 mg, 0.59 mmol), Cs₂ CO₃ (2410 mg, 7.40 mmol) and 1,4-dioxane (10 ml). The reaction was carried out under nitrogen protection at 100° C. for 16 hours. After completion of the reaction, the reaction solution was brought to room temperature, decanted into water (40 ml) and extracted three times with DCM (30 ml). The organic phases were combined, washed with saturated brine, dried over anhydrous sodium sulfate, filtered, and concentrated to dryness. The residue was purified by preparative plate with the unfolding agent ratio of DCM/MeOH=10/1 to afford the compound TPD12163-2 (240 mg, yellow solid, purity 86.393%), yield: 13.64%. LCMS (ESI) m/z calcd. for C₂₅H₃₂N₆O₆ [M+H]⁺ 513.24; found 513.1; ¹H NMR (400 MHz, DMSO_d₆): δ=11.18-11.01 (m, 2H), 8.67 (s, 1H), 8.33 (s, 1H), 5.10 (dd, J=13.3, 5.0 Hz, 1H), 4.53 (d, J=18.4 Hz, 1H), 4.39 (d, J=18.4 Hz, 1H), 4.15 (d, J=10.1 Hz, 1H), 3.52 (s, 1H), 3.22 (d, J=14.2 Hz, 1H), 3.07 (dd, J=15.9, 8.9 Hz, 1H), 2.96-2.87 (m, 1H), 2.81 (d, J=7.3 Hz, 3H), 2.64-2.52 (m, 3H), 2.37 (dd, J=13.0, 4.4 Hz, 1H), 2.03-1.96 (m, 1H), 1.71 (d, J=9.2 Hz, 1H), 1.62 (t, J=9.6 Hz, 1H), 1.38 (s, 9H), 1.20 (d, J=24.3 Hz, 1H). The compound structure is confirmed.

To a 25 ml single-necked vial was sequentially added compound TPD12163-2 (240 mg, 0.47 mmol) and 4 N HCl/1,4-dioxane (8 ml). The reaction was carried out at 25° C. for 2 h. After completion of the reaction, it was concentrated to dryness to give the crude compound TPD12163-3 (240 mg, yellow solid, purity 75.179%), yield: 93.62%. LCMS (ESI) m/z calcd. for C₂₀H₂₄N₆O₄ [M+H]⁺ 413.19; found 413.1. The compound structure is confirmed.

To a 25 ml single-necked vial were sequentially added compound TPD12163-3 (108 mg, 0.26 mmol), compound TPD12086-9 (150 mg, 0.26 mmol), sodium triacetoxyborohydride (111 mg, 0.53 mmol) and DCE (3 ml). The reaction was carried out under nitrogen protection at 25° C. for 16 hours. After the reaction was completed, the reaction solution was poured into water (10 ml) and an appropriate amount of saturated NaHCO₃ solution was added to make the reaction solution alkaline. It was extracted three times with DCM (5 ml). The organic phases were combined, washed with saturated brine, dried over anhydrous sodium sulfate, filtered, and concentrated to dryness. The residue was prepared by high performance liquid chromatography (HPLC) with the following parameters: column: XBridge-1 5 μm 19-150 mm; mobile phase: acetonitrile-water (0.1% FA); gradient: 10-55/8 min to afford the compound TPD12163 (9.04 mg, yellow solid, purity 92.832%) in the following yield: 3.08%. LCMS (ESI) m/z calcd. for C₅₂H₅₂F₂N₁₀O₇ [M+H]⁺ 967.40; found 967.3; ¹H NMR (400 MHz, DMSO_d₆): δ=11.36 (s, 1H), 11.02 (s, 1H), 10.20 (s, 1H), 10.07 (s, 1H), 8.68 (s, 1H), 8.56 (d, J=5.1 Hz, 1H), 8.34 (s, 1H), 7.85 (d, J=13.5 Hz, 1H), 7.76 (d, J=8.7 Hz, 2H), 7.68-7.56 (m, 2H), 7.44 (d, J=8.3 Hz, 1H), 7.28-7.06 (m, 4H), 6.45 (d, J=5.1 Hz, 1H). 5.14-5.04 (m, 1H), 4.54 (d, J=18.3 Hz, 1H), 4.39 (d, J=18.1 Hz, 1H), 3.33 (s, 3H), 2.96-2.87 (m, 2H), 2.77 (d, J=7.1 Hz, 4H), 2.71-2.60 (m, 3H), 2.54 (s, 2H). 2.44-2.31 (m, 3H), 2.09-1.83 (m, 4H), 1.60 (d, J=32.7 Hz, 3H), 1.48-1.23 (m, 7H). The compound structure is confirmed.

### Example 85: Synthesis of Compound TPD12205

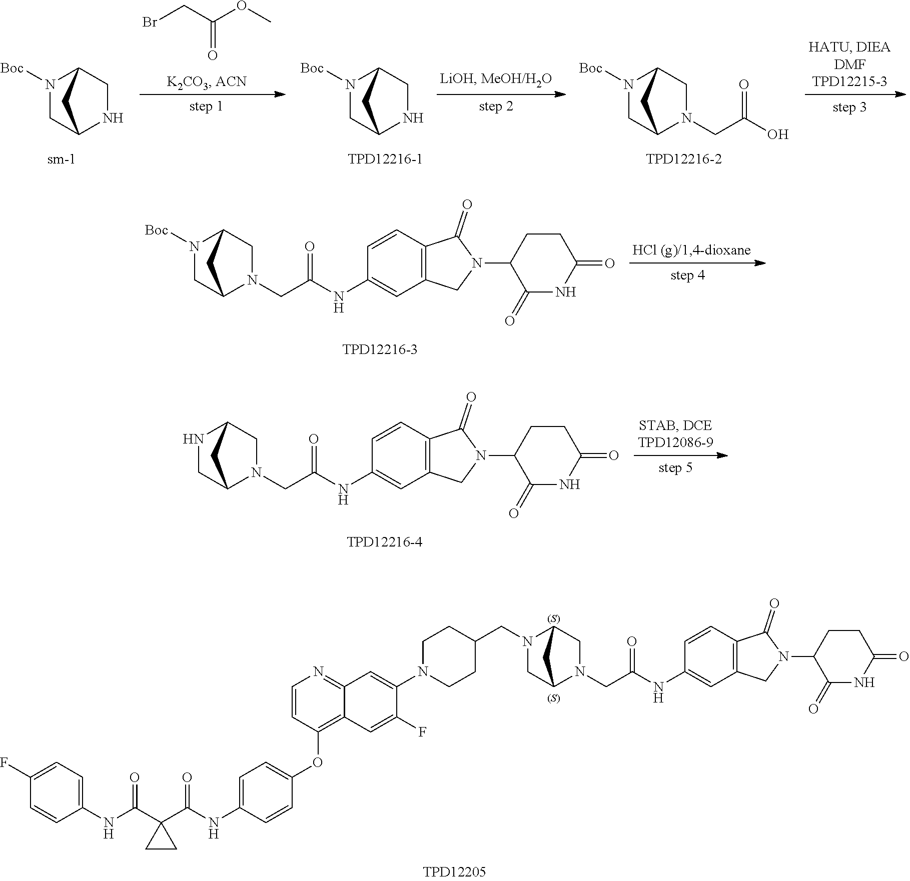

To a 25 ml single-necked vial were sequentially added compound sm-1 (1000 mg, 5.02 mmol), K₂CO₃ (1040 mg, 7.53 mmol),methyl bromoacetate (768 mg, 5.02 mmol) and ACN (15 ml). The reaction was carried out under nitrogen protection at 80° C. for 16 hours. After the reaction was completed, the reaction solution was reduced to room temperature, poured into water (20 ml), extracted with EtOAc (60 ml) for three times, the reaction solution was concentrated to dryness, and the crude product was purified by silica gel chromatographic column, the ratio of the unfolding agent was D/M=100/1˜70/1, to obtain the compound TPD12216-1 (1200 mg, white solid, purity 73.225%), the yield: 64.53%; LCMS (ESI) m/z calcd. for C₁₃H₂₂N₂O₄ [M+H]⁺ 271.16; found 271.2; ¹H NMR (400 MHz, DMSO_d₆): δ=4.13 (d, J=11.9 Hz, 1H), 3.60 (s, 3H), 3.49 (d, J=3.5 Hz, 1H), 3.38 (s 2H), 3.29-3.23 (m, 1H), 3.10-3.03 (m, 1H), 2.87-2.80 (m, 1H), 2.55-2.51 (m, 1H), 1.75-1.70 (m, 1H), 1.63-1.54 (m, 1H), 1.39 (s, 9H). The compound structure is confirmed.

To a 1000 ml single-necked vial was sequentially added compound TPD 12216-1 (1300 mg, 4.79 mmol), lithium hydroxide monohydrate (603 mg, 14.37 mmol) and MeOH/H₂O=2/1 (15 ml). The reaction was carried out under nitrogen protection at 25° C. for 16 hours. After the reaction was completed, the reaction solution was brought to room temperature, spun dry as a solid, decanted into water (12 mL), adjusted to a pH of 7 with hydrochloric acid, saturated with salt, and then extracted eight times with EA (60 ml) and THF (60 ml). The organic phases were combined, washed with saturated brine, dried over anhydrous sodium sulfate, filtered, and concentrated to dryness. The compound TPD12216-2 (1100 mg, yellow oil, purity 46.144%) was obtained, yield: 41.17%; LCMS (ESI) m/z calcd. for C₁₂H₂₀N₂O₄ [M+H]⁺ 257.14; found 257.3; ¹H NMR (400 MHz, DMSO_d₆): δ=4.15 (d, J=12.9 Hz, 1H), 3.66 (d, J=11.4 Hz, 2H), 3.18-3.06 (m, 3H), 2.94-2.87 (m, 1H), 2.67 (t, J=11.4 Hz, 1H), 1.89-1.81 (m, 1H), 1.71-1.59 (m, 1H), 1.39 (s, 9H). The compound structure is confirmed.

To a 25 ml single-necked vial was sequentially added compound TPD12216-2 (1.00 g, 3.89 mmol), compound TPD12215-3 (1.01 g, 3.89 mmol), HATU (2.96 g, 7.77 mmol), DIEA (1.01 g, 7.77 mmol), and DMF (20 ml). The reaction was carried out at 50° C. for 16 h. After the reaction was completed, it was reduced to room temperature, poured into water (100 ml) and extracted with EtOAc (100 ml) for five times, and the reaction solution was concentrated to dryness, and the crude product was purified by silica gel chromatographic column, the ratio of unfolding reagent was DCM/MeOH=50/1˜15/1, and the crude was prepared by high-performance liquid chromatography (HPLC), with the relevant parameters as follows: chromatographic column: XBridge-1 5 μm 19-150 mm; mobile phase: acetonitrile-water (0.1% FA); gradient: 10-55/8 min, after the crude compound TPD12216-3 (40 mg, yellow solid, purity 83.183%) was obtained, yield: 1.72%. LCMS (ESI) m/z calcd. for C₂₅H₃₁N₅O₆ [M+H]⁺ 498.23; found 498.1. found 498.1. The compound structure is confirmed.

Compound TPD12216-3 (30 mg, 0.060 mmol) 4 N HCl/1,4-dioxane (4 ml) was added sequentially in a 25 ml single-necked vial. The reaction was carried out at 25° C. for 2 h. After completion of the reaction, it was concentrated to dryness to obtain the crude compound TPD12216-4 (25 mg, yellow solid, purity 95.206%) in 99.50% yield; LCMS (ESI) m/z calcd. for C₂₀H₂₃N₅O₄ [M+H]⁺ 399.18; found 399.6. The compound structure is confirmed.

To a 25 ml single-necked vial was sequentially added compound TPD12215-5 (40 mg, 0.101 mmol), compound TPD12086-9 (57 mg, 0.101 mmol), sodium triacetoxyborohydride (43 mg, 0.201 mmol) and DCE (1 ml). The reaction was carried out under nitrogen protection at 25° C. for 16 hours. After the reaction was completed, the reaction solution was poured into water (3 ml) and an appropriate amount of saturated NaHCO₃ solution was added to make the reaction solution alkaline. It was extracted three times with DCM (15 ml). The organic phases were combined, washed with saturated brine, dried over anhydrous sodium sulfate, filtered, and concentrated to dryness. The residue was prepared by high performance liquid chromatography (HPLC) with the following parameters: column: XBridge-1 5 μm 19-150 mm; mobile phase: acetonitrile-water (0.1% FA); gradient: 10-55/8 min to give compound TPD12205 (3.41 mg, yellow solid, purity 96.597%), yield: 3.28%; LCMS (ESI) m/z calcd. for C₅₂H₅₁F₂N₉O₇ [M+H]⁺ 952.39; found 952.3; ¹H NMR (400 MHz, DMSO_d₆): δ=10.97 (s, 1H), 10.18 (s, 1H), 10.04 (s, 1H), 9.98 (s, 1H), 8.56 (d, J=5.2 Hz, 1H). 8.21 (s, 1H), 8.02 (d, J=8.0 Hz, 1H), 7.85 (d, J=13.4 Hz, 1H), 7.80-7.71 (m, 3H), 7.70-7.59 (m, 3H), 7.44 (d, J=8.5 Hz, 1H), 7.23-7.11 (m, 4H), 6.45 (d, J=5.1 Hz, 1H), 5.09 (dd, J=13.2, 4.9 Hz, 1H), 4.43 (d, J=17.1 Hz, 1H), 4.30 (d, J=17.4 Hz, 1H), 3.68-3.47 (m, 4H), 2.96-2.85 (m, 1H), 2.83-2.53 (m, 7H), 2.42-2.24 (m, 5H), 2.03-1.95 (m, 1H), 1.92-1.86 (m, 2H), 1.74 (d, J=9.9 Hz, 3H), 1.65 (d, J=8.1 Hz, 1H), 1.56 (s, 1H), 1.47 (s, 4H), 1.39-1.30 (m, 2H). The compound structure is confirmed.

### Example 86: Synthesis of Compound TPD12215

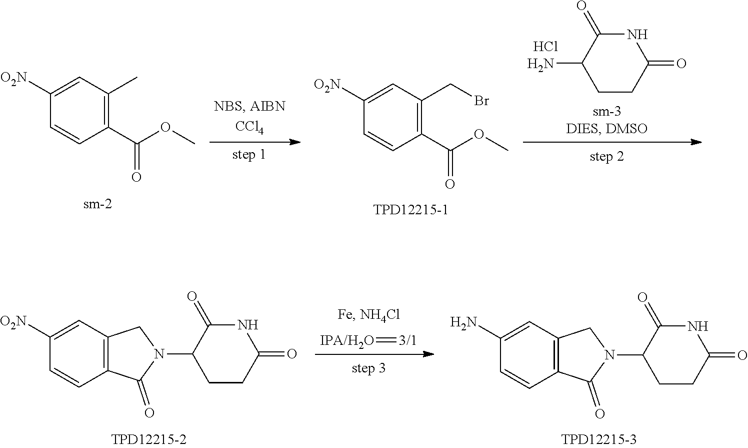

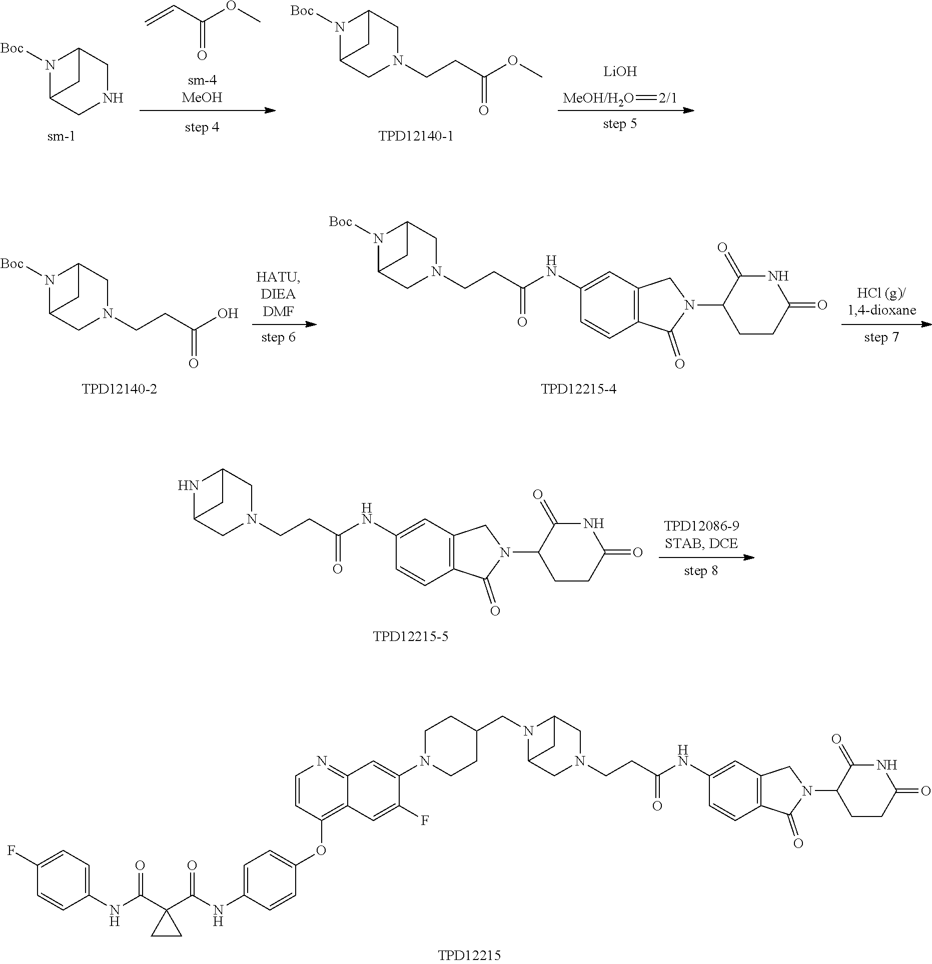

To a 500 ml single-necked vial were sequentially added compound sm-2 (20.0 g, 0.10 mol), NBS (27.4 g, 0.15 mol), AIBN (1.68 g, 10.3 mmol) and carbon tetrachloride (250 ml). The reaction was carried out at 70° C. for 16 h under nitrogen protection. After completion of the reaction, the reaction solution was reduced to room temperature, decanted into water (200 ml), extracted twice with DCM (200 ml) and the reaction solution was concentrated to dryness to give compound TPD12215-1 (34 g, yellow solid, purity 58.534%), yield: 70.83%; LCMS (ESI) m/z calcd. for C₉H₈BrNO₄ [M+H]⁺ 273.96/275.96; found 275.0/276.0; ¹H NMR (400 MHz, DMSO_d₆): δ=8.16-7.97 (m, 3H), 5.10 (s, 2H), 3.96 (s, 3H). The compound structure is confirmed.

To a 1000 ml single-necked vial was sequentially added compound TPD12215-1 (42 g crude, 0.10 mol), 3-amino-2,6-piperidinedione hydrochloride (30.26 g, 0.18 mol), DIEA (39.6 g, 0.31 mol) and DMSO (450 mL). The reaction was carried out under nitrogen protection at 100° C. for 16 hours. After the reaction was completed, the reaction solution was reduced to room temperature, decanted into water (2500 ml) and extracted eight times with EA (3000 ml). The organic phases were combined, washed with saturated brine, dried over anhydrous sodium sulfate, filtered, and concentrated to dryness. The residue was purified by column chromatography with eluent ratio of DCM/MeOH=100/1˜20/1 to afford compound TPD12215-2 (11.75 g, green solid, purity 90.781%), yield: 24.09%; LCMS (ESI) m/z calcd. for C₁₃H₁₁N₃O₅ [M+H]⁺ 290.07; found 289.9; ¹H NMR (400 MHz, DMSO_d₆): δ=11.06 (s, 1H), 8.53 (s, 1H), 8.36 (dd, J=8.3, 1.7 Hz, 1H), 7.98 (d, J=8.3 Hz, 1H), 5.18 (dd, J=13.3, 5.1 Hz, 1H), 4.61 (d, J=18.1 Hz, 1H), 4.50 (d, J=18.1 Hz, 1H), 2.98-2.87 (m, 1H), 2.50-2.38 (m, 2H), 2.11-2.00 (m, 1H). The compound structure is confirmed.

To a 100 ml single-necked vial were sequentially added compound TPD12215-2 (3 g, 0.0104 mol), iron powder (1.16 g, 0.0208 mol), ammonium chloride (1.11 g, 0.0208 mmol), isopropanol (180 ml) and H₂ O (60 ml). The reaction was carried out under nitrogen protection at 80° C. for 16 hours. After the reaction was completed, the reaction solution was reduced to room temperature, filtered, concentrated to dryness, and the residue was purified by chromatographic column with eluent ratio of DCM/MeOH=100/1˜20/1 to obtain the compound TPD12215-3 (770 mg, yellow solid, purity 94.466%), yield: 26.92%; LCMS (ESI) m/z calcd. for C₁₃H₁₃N₃O₃ [M+H]⁺ 260.10; found 260.1; ¹H NMR (400 MHz, DMSO_d₆): δ=10.93 (s, 1H), 7.41-7.26 (m, 1H), 6.64-6.61 (m, 1H), 5.92-5.64 (m, 2H), 5.01 (dd, J=13.3, 5.0 Hz. 1H). 4.37-4.02 (m, 2H), 2.97-2.83 (m, 1H), 2.60-2.53 (m, 2H), 2.40-2.26 (m, 1H), 1.99-1.88 (m, 1H). The compound structure is confirmed.

To a 50 ml single-necked vial was sequentially added compound TPD12140-2 (610 mg, 2.25 mmol), compound TPD12215-3 (583 mg, 2.25 mmol), HATU (1.71 g, 4.50 mmol), DIEA (581 mg, 4.50 mmol) and DMF (10 ml). The reaction was carried out at 50° C. for 16 h. After completion of the reaction, it was reduced to room temperature, decanted into water (60 ml) and extracted with EA (160 ml) for eight times, and the residue was purified by a column with the eluent ratio of DCM/MeOH=100/1˜20/1 to give the crude compound TPD12215-4 (500 mg, yellow solid, purity 86.189%), yield: 37.39%; LCMS (ESI) m/z calcd. for C₂₁H₂₅N₅O₄ [M+H]⁺ 512.24; found 512.2; ¹H NMR (400 MHz, DMSO_d₆): δ=11.03-10.90 (m, 1H), 10.41-10.28 (m, 1H), 8.76 (d, J=4.2 Hz, 1H), 8.53 (d, J=8.4 Hz, 1H), 7.55-7.47 (m, 1H). 4.54-4.22 (m, 3H), 4.14-4.07 (m, 3H), 4.05-3.94 (m, 3H), 2.95-2.89 (m, 5H), 2.69-2.57 (m, 3H), 2.37-2.29 (m, 2H), 1.39 (s, 9H). The compound structure is confirmed.

Compound TPD12215-4 (110 mg, 0.21 mmol) 4 N HCl/1,4-dioxane (8 ml) was added sequentially in a 25 ml single-necked vial. The reaction was carried out at 25° C. for 2 h. After completion of the reaction, it was concentrated to dryness to give the crude compound TPD12215-5 (100 mg, yellow solid, purity 89.832%), yield: 86.44%; LCMS (ESI) m/z calcd. for C₂₁H₂₅N₅O₄ [M+H]⁺ 412.19; found 412.2. The compound structure is confirmed.

To a 25 ml single-necked vial were sequentially added compound TPD12215-5 (120 mg, 0.29 mmol), compound TPD12086-9 (166 mg, 0.29 mmol), sodium triacetoxyborohydride (124 mg, 0.58 mmol) and DCE (3 ml). The reaction was carried out under nitrogen protection at 25° C. for 16 hours. After the reaction was completed, the reaction solution was poured into water (3 ml) and an appropriate amount of saturated NaHCO₃ solution was added to make the reaction solution alkaline. It was extracted three times with DCM (15 ml). The organic phases were combined, washed with saturated brine, dried over anhydrous sodium sulfate, filtered, and concentrated to dryness. The residue was prepared by high performance liquid chromatography (HPLC) with the following parameters: column: XBridge-1 5 μm 19-150 mm; mobile phase: acetonitrile-water (0.1% FA); gradient: 10-55/8 min to give compound TPD12215 (16 mg, yellow solid, purity 97%), yield: 5%; LCMS (ESI) m/z calcd. for C₅₄H₅₃F₄N₉O₈ [M+H]⁺ 966.40; found 966.3; ¹H NMR (400 MHz, DMSO_d₆): δ=11.01 (s, 1H), 10.39 (s, 1H), 10.20 (s, 1H), 10.06 (s, 1H), 8.56 (d, J=5.2 Hz, 1H), 8.21 (s, 2H)), 7.99 (s, 1H), 7.86 (d, J=13.4 Hz, 1H), 7.76 (d, J=8.8 Hz, 2H), 7.70-7.56 (m, 4H), 7.44 (d, J=8.4 Hz, 1H), 7.22 (d, J=8.9 Hz, 2H), 7.19-7.11 (m, 2H), 6.45 (d, J=5.2 Hz, 1H). 5.12-5.03 (m, 1H), 4.42 (d, J=17.3 Hz, 1H), 4.29 (d, J=17.3 Hz, 1H), 3.67 (s, 2H), 3.54 (d, J=11.4 Hz, 2H), 3.09-2.65 (m, 10H), 2.63-2.53 (m, 3H), 2.43-2.26 (m, 3H), 2.02-1.80 (m, 4H), 1.56 (s, 1H), 1.47 (s, 4H), 1.41-1.32 (m, 2H). The compound structure is confirmed.

### Example 87: Synthesis of Compound TPD12216

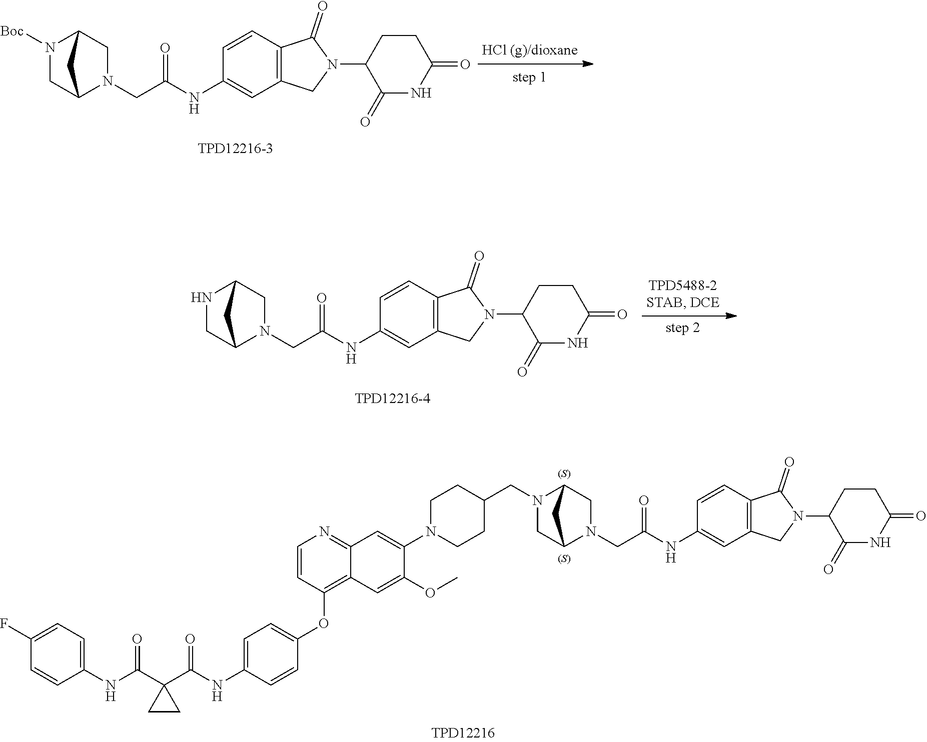

Compound TPD12216-3 (55 mg, 0.11 mmol) 4 N HCl/1,4-dioxane (8 ml) was added sequentially in a 25 ml single-necked vial. The reaction was carried out at 25° C. for 1.5 h. After completion of the reaction, it was concentrated to dryness to give the crude compound TPD12216-4 (50 mg, white solid, purity 83.291%) in 80.24% yield. LCMS (ESI) m/z calcd. for C₂₀H₂₃N₅O₄ [M+H]⁺ 398.18; found 398.1. The compound structure is confirmed.

To a 25 ml single-necked vial were sequentially added compound TPD12216-4 (70 mg, 0.18 mmol), compound TPD5488-2 (103 mg, 0.18 mmol), sodium triacetoxyborohydride (75 mg, 0.35 mmol) and DCE (2 ml). The reaction was carried out under nitrogen protection at 25° C. for 16 hours. After the reaction was completed, the reaction solution was poured into water (3 ml) and an appropriate amount of saturated NaHCO₃ solution was added to make the reaction solution alkaline. It was extracted three times with DCM (15 ml). The organic phases were combined, washed with saturated brine, dried over anhydrous sodium sulfate, filtered, and concentrated to dryness. The residue was prepared by high performance liquid chromatography (HPLC) with the following parameters: column: XBridge-1 5 μm 19-150 mm; mobile phase: acetonitrile-water (0.1% FA); gradient: 10-55/8 min to give compound TPD12216 (2.17 mg, yellow solid, purity 87%) in 0.19% yield. LCMS (ESI) m/z calcd. for C₅₃H₅₄FN₉O₈ [M+H]⁺ 963.41; found 482.9; ¹H NMR (400 MHz, DMSO_d₆): δ=10.97 (s, 1H), 10.18 (s, 1H), 10.07-9.97 (m, 1H), 8.43 (d, J=4.9 Hz, 1H), 8.27 (s, 2H). 8.07-7.93 (m, 1H), 7.79-7.70 (m, 3H), 7.68-7.58 (m, J=15.3, 6.8 Hz, 3H), 7.46 (s, 1H), 7.32 (s, 1H), 7.23-7.10 (m, 4H), 6.39 (d, J=4.7 Hz, 1H), 5.14-5.03 (m 1H), 4.43 (d, J=17.7 Hz, 1H), 4.35-4.24 (m, 1H), 3.94 (s, 3H), 3.63-3.55 (m, 3H), 3.49-3.40 (m, 5H), 3.37-3.30 (m, 5H), 2.75-2.62 (m, 5H), 2.42-2.31 (m, 3H), 2.03-1.95 (m, 1H), 1.88-1.85 (m, 1H), 1.74 (d, J=9.7 Hz, 1H), 1.66-1.60 (m, 1H), 1.47 (s, 3H), 1.39-1.32 (m, 2H). The compound structure is confirmed.

### Example 88: Synthesis of Compound TPD12218

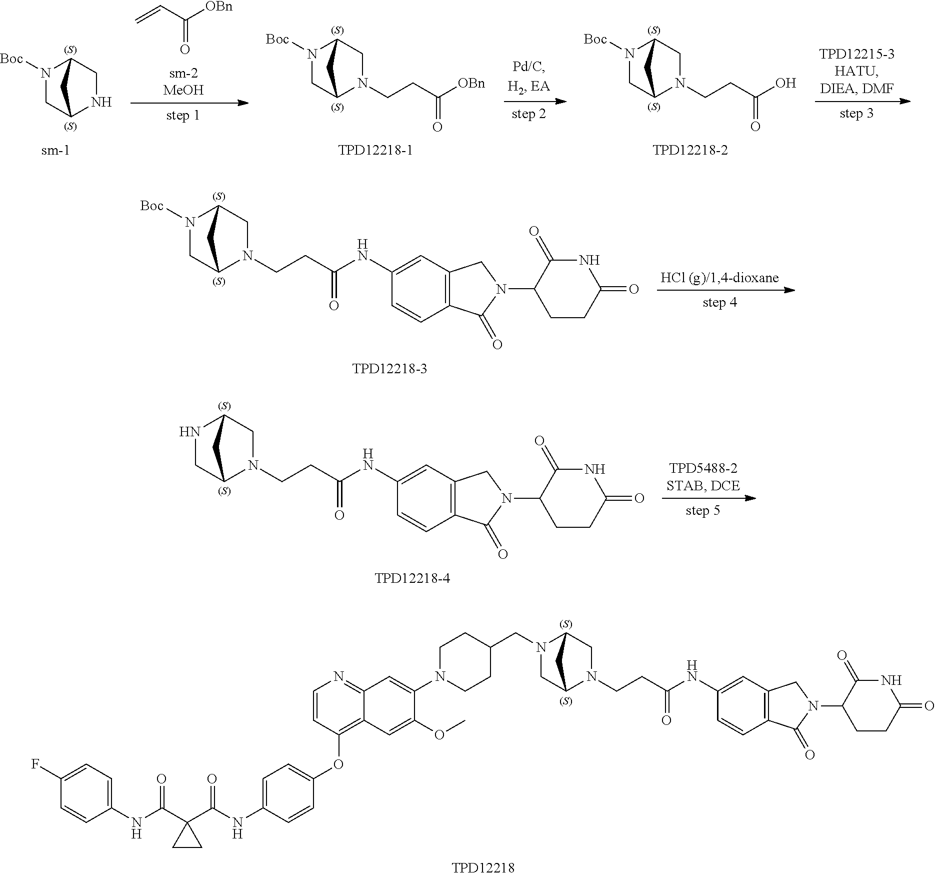

Compounds sm-1 (1.0 g, 5.02 mmol), sm-2 (1.22 g, 7.53 mmol) and methanol (15 ml) were added sequentially in a 50 ml single-necked vial. The reaction was carried out under nitrogen protection at 20° C. for 16 hours. After completion of the reaction, the reaction solution was concentrated to dryness and the residue was purified by column chromatography with eluent ratio of DCM/MeOH=100/1-60/1 to afford the compound TPD12218-1 (950 mg, colorless oily substance, purity 97.200%), yield: 50.91%. LCMS (ESI) m/z calcd. for C₂₀H₂₈N₂O₄ [M+H]⁺ 361.2; found 361.2; ¹H NMR (400 MHz, CDCl₃): δ=7.41-7.28 (m, 5H), 5.19-5.09 (m, 2H), 4.27 (d, J=51.6 Hz, 1H), 3.54-3.38 (m, 2H), 3.13 (t, J=10.3 Hz, 1H), 2.96-2.82 (m, 3H), 2.65-2.44 (m, 3H), 1.82-1.74 (m, 1H), 1.71-1.65 (m, 1H), 1.45 (s, 9H). The compound structure is confirmed.

To a 25 ml vial was added compound TPD12218-1 (900 mg, 2.49 mmol), palladium carbon (100 mg, 10%) and ethyl acetate (10 ml) in sequence. 1 atm hydrogen pressure reacted at 20° C. for 16 h. After completion of the reaction, the filtrate was spun dry to give compound TPD12218-2 (500 mg, colorless oil, purity: 70.31%) in yield: 70.31%. After completion of the reaction, the filtrate was filtered and the filtrate was spun dry to give the compound TPD12218-2 (500 mg, colorless oil, purity 95%), yield: 70.31%. LCMS (ESI) m/z calcd. for C₁₃H₂₂N₂O₄ [M+H]⁺ 271.2; found 271.2; ¹H NMR (400 MHz, DMSO_d₆): δ=4.14 (d, J=11.5 Hz, 1H), 3.50 (s, 1H), 3.32-3.22 (m, 1H), 3.17 (s, 1H), 3.12-3.01 (m, 1H), 2.84-2.64 (m, 3H), 2.49-2.42 (m, 1H), 2.29 (t, J=7.0 Hz, 2H), 1.75-1.57 (m, 2H), 1.39 (s, 8H).

To a 25 ml single-necked vial were sequentially added compound TPD12218-2 (400 mg, 1.47 mmol), compound TPD12215-3 (382 mg, 1.47 mmol), HATU (1.12 g, 2.95 mmol), DIEA (572 mg, 2.95 mmol) and DMF (5 ml). The reaction was carried out under nitrogen protection at 70° C. for 16 hours. After the reaction was completed saturated aqueous sodium bicarbonate solution (20 ml) was added and the aqueous phase was extracted three times with EA (20 ml). The organic phases were combined, washed with saturated brine, dried over anhydrous sodium sulfate, filtered, and concentrated to dryness, and the residue was purified by column chromatography with an eluent ratio of DCM/MeOH=50/1˜30/1 to afford the compound TPD12218-3 (60 mg, yellow solid, purity 81.748%), yield: 4.33%. LCMS (ESI) m/z calcd. for C₂₆H₃₃N₅O₆ [M+H]⁺ 512.2; found 512.1. The compound structure is confirmed.

Compound TPD12218-3 (60 mg, 0.117 mmol) and 1,4-dioxane (4 ml, 4 N) were added sequentially in a 25 ml single-necked vial. The reaction was carried out under nitrogen protection at 20° C. for 4 hours. The reaction was completed and concentrated to dryness to give compound TPD12218-4 dihydrochloride (60 mg, yellow solid, purity 92.356%), yield: 97.69%. LCMS (ESI) m/z calcd. for C₂₁H₂₅N₅O₄ [M+H]⁺ 411.2; found 411.2. The compound structure is confirmed.

To a 25 ml single-necked vial was sequentially added compound TPD12218-4 (58 mg, 0.12 mmol), compound TPD5488-2 (70 mg, 0.12 mmol), sodium triacetoxyborohydride (76 mg, 0.36 mmol) and DCE (4 ml). The reaction was carried out under nitrogen protection at 20° C. for 16 hours. After the reaction was completed, the reaction solution was poured into saturated aqueous sodium bicarbonate solution (10 ml). The organic phase was separated and the aqueous phase was extracted three times with DCM (10 ml). The organic phases were combined, washed with saturated brine, dried over anhydrous sodium sulfate, filtered, and concentrated to dryness. The residue was separated by preparative plate (DCM/MeOH=8/1) to afford compound TPD12218 (23.5 mg, yellow solid, purity 97.638%), yield: 17.32%. LCMS (ESI) m/z calcd. for C₅₄H₅₆FN₉O₈ [M+H]⁺ 978.4; found 490.2; 979.4; ¹H NMR (400 MHz, DMSO_d₆): δ=11.01 (s, 1H), 10.54 (s, 1H), 10.18 (s, 1H), 10.06 (s, 1H), 8.43 (d, J=5.2 Hz, 1H), 7.97 (s, 1H), 7.76 (d, J=8.8 Hz, 2H), 7.71-7.57 (m, 4H). 7.46 (s, 1H), 7.32 (s, 1H), 7.27-7.11 (m, 4H), 6.39 (d, J=5.2 Hz, 1H), 5.09 (dd, J=13.2, 5.0 Hz, 1H), 4.43 (d, J=17.4 Hz, 1H), 4.29 (d, J=17.4 Hz, 1H), 3.94 (s 3H), 3.59 (d, J=10.9 Hz, 2H), 3.47 (d, J=26.9 Hz, 3H), 2.99-2.72 (m, 7H), 2.70-2.54 (m, 5H), 2.44-2.29 (m, 2H), 2.04-1.95 (m, 1H), 1.91-1.81 (m, 2H), 1.71 (s, 2H), 1.63-1.52 (m, 1H), 1.47 (s, 4H), 1.42-1.29 (m, 2H). The compound structure is confirmed.

### Example 89: Synthesis of Compound TPD12234

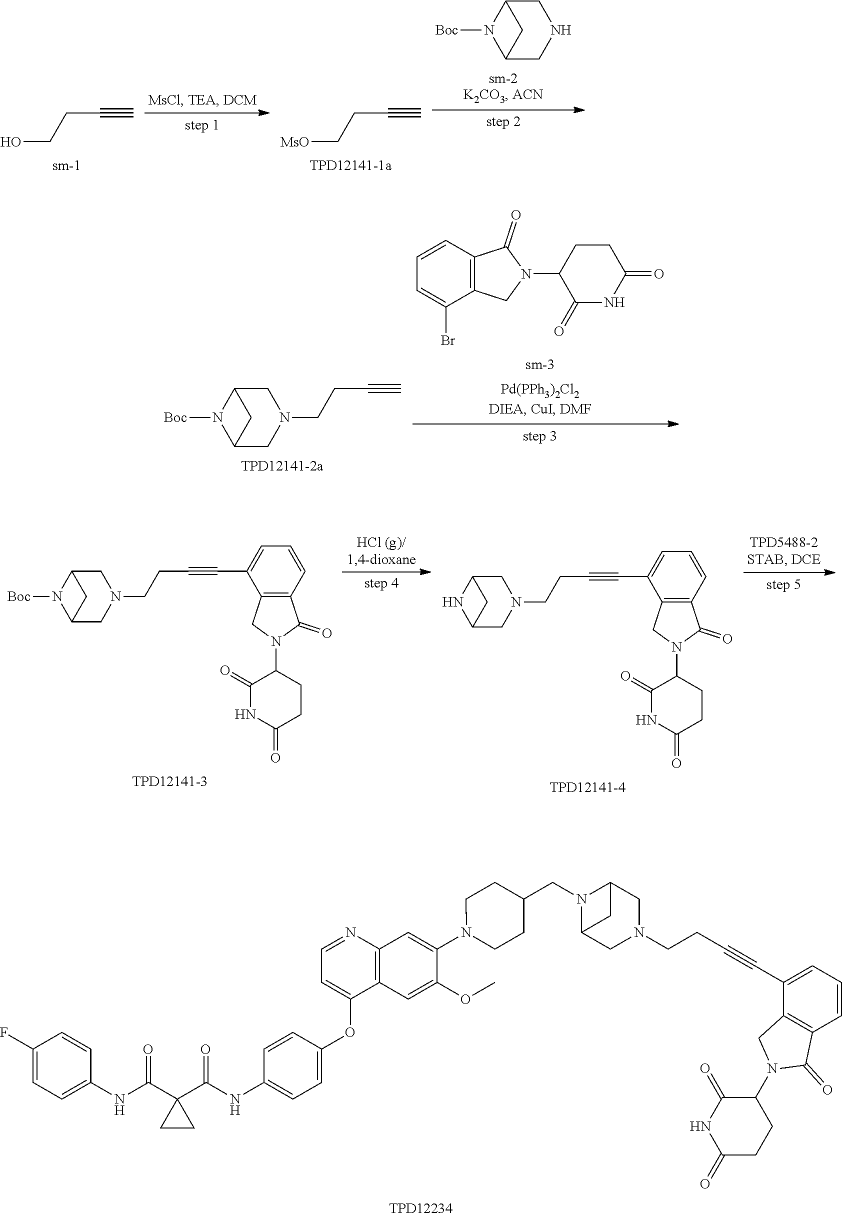

To a 250 ml three-necked vial was added sequentially compound sm-1 (5 g, 71.3 mmol), DCM (60 ml) and TEA (8.66 g, 85.6 mmol). MsCl (8.94 g, 78.5 mmol) was added dropwise at 0° C. The reaction was raised to 20° C. for 2 hours. After the reaction was completed, the reaction was quenched with water (100 ml), the organic phase was separated and washed with saturated brine, dried with anhydrous sodium sulfate, filtered, and concentrated to dryness, and the residue was purified by silica gel chromatographic column with an eluent ratio of PE/EtOAc=30/1˜10/1 to afford the compound TPD12141-1a (1.6 g, colorless oily substance), yield: 13.62%. LCMS (ESI) m/z calcd. for C₅H₈O₃S [M+H]⁺ 149.02; found 149.0; ¹H NMR (400 MHz, CDCl₃): δ=4.32 (t, J=6.7 Hz, 2H), 3.07 (s, 3H), 2.67 (td, J=6.7, 2.6 Hz, 2H), 2.08 (t, J=2.6 Hz, 1H). The compound structure is confirmed.

To a 100 ml single-necked vial were sequentially added compounds TPD12141-1a (1.19 g, 8.03 mmol), ACN (20 ml), sm-2 (1.6 g. 8.03 mmol) and potassium carbonate (1.66 g, 12.04 mmol). The reaction was carried out under nitrogen protection at 80° C. for 16 hours. After completion of the reaction, the reaction solution was reduced to room temperature, water (80 ml) was added and extracted three times with EA (50 ml). The organic phases were combined, washed with saturated brine, dried over anhydrous sodium sulfate, filtered, and concentrated to dryness, and the residue was purified by silica gel chromatographic column with eluent ratio of DCM/MeOH=50/1˜30/1 to afford the compound TPD12141-2a (0.8 g, yellow solid, purity 81.061%), yield: 32.13%. LCMS (ESI) m/z calcd. for C₁₄H₂₂N₂O₂ [M+H]⁺ 251.17; found 250.9; ¹H NMR (400 MHz, CDCl₃): δ=4.05 (br. s, 2H), 3.30-2.97 (m, 2H), 2.93-2.71 (m, 5H), 2.43-2.32 (m, 3H), 1.97 (t, J=2.6 Hz, 1H), 1.65 (d, J=7.9 Hz, 1H), 1.45 (s, 9H). The compound structure is confirmed.

To a 100 ml single-necked vial was sequentially added compound TPD12141-1a (747 mg, 2.97 mmol), DMF (16 ml), sm-3 (800 mg, 2.48 mmol), Pd(PPh)₃₂ Cl₂ (174 mg, 0.25 mmol), iodinated ketone (24 mg, 0.12 mmol) and DIEA (640 mg, 4.95 mmol). The reaction was carried out under nitrogen protection at 80° C. for 16 hours. After completion of the reaction, the reaction solution was reduced to room temperature, water (80 ml) was added and extracted three times with EA (50 ml). The organic phases were combined, washed with saturated brine, dried over anhydrous sodium sulfate, filtered, and concentrated to dryness, and the residue was purified by silica gel chromatographic column with eluent ratio of DCM/MeOH=50/1˜30/1 to afford the compound TPD12141-3 (460 mg, yellow solid, purity 90.450%) in the yield: 34.05%. LCMS (ESI) m/z calcd. for C₂₇H₃₂N₄O₅ [M+H]⁺ 493.24; found 493.1; ¹H NMR (400 MHz, DMSO_d₆): δ=11.02 (s, 1H), 7.71 (d, J=7.4 Hz, 1H), 7.63 (d, J=7.4 Hz, 1H), 7.52 (t, J=7.6 Hz, 1H). 5.16 (dd, J=13.3, 5.0 Hz, 1H), 4.46 (d, J=17.8 Hz, 1H), 4.30 (d, J=17.7 Hz, 1H), 3.97 (d, J=5.7 Hz, 2H), 3.18-2.99 (m, 2H), 2.97-2.76 (m, 5H), 2.68-2.56 (m, 3H), 2.46-2.36 (m, 1H), 2.34-2.23 (m, 1H), 2.06-1.97 (m, 1H), 1.54 (d, J=7.9 Hz, 1H), 1.37 (s, 9H). The compound structure is confirmed.

To a 50 ml single-necked vial was sequentially added compound TPD12141-3 (60 mg, 0.12 mmol) and HCl(g)/1,4-dioxane (4 ml). the reaction was carried out at 20° C. for 2 h. After the reaction was completed, the reaction solution was concentrated to dryness to give compound TPD12141-4 (65 mg, yellow solid), purity: 92.52%. After completion of the reaction, the reaction solution was concentrated to dryness to obtain compound TPD12141-4 (65 mg, yellow solid, purity 80.563%), yield: 92.52%. LCMS (ESI) m/z calcd. for C₂₂H₂₄N₄O₃ [M+H]⁺ 393.19; found 393.1. The compound structure is confirmed.

To a 25 ml single-necked vial was sequentially added compound TPD5488-2 (70 mg, 0.12 mmol), compound TPD12141-4 (56 mg, 0.12 mmol), sodium triacetoxyborohydride (76 mg, 0.36 mmol) and DCE (4 ml). The reaction was carried out under nitrogen protection at 20° C. for 16 hours. After completion of the reaction, the reaction solution was decanted into aqueous saturated NaHCO₃ solution (10 ml). It was extracted three times with DCM (20 ml). The organic phases were combined, washed with saturated brine, dried over anhydrous sodium sulfate, filtered, and concentrated to dryness. The residue was separated by preparative plate with unfolding agent ratio DCM/MeOH=10/1 to give compound TPD12234 (11.5 mg, off-white solid, purity 99.127%), yield: 9.91%. LCMS (ESI) m/z calcd. for C₅₅H₅₅FN₈O₇ [M+H]⁺ 959.42; found 480.3. 961.3; ¹H NMR (400 MHz, DMSO_d₆): δ=11.03 (s, 1H), 10.18 (s, 1H), 10.06 (s, 1H), 8.43 (d, J=5.2 Hz, 1H), 8.26 (s, 1H), 7.81-7.68 (m, 3H), 7.68-7.59 (m, 3H), 7.53 (t, J=7.5 Hz, 1H), 7.46 (s, 1H), 7.30 (s, 1H), 7.25-7.10 (m, 4H), 6.39 (d, J=5.1 Hz, 1H), 5.16 (dd, J=13.3, 5.1 Hz, 1H), 4.46 (d, J=17.7 Hz, 1H), 4.31 (d, J=17.6 Hz, 1H), 3.94 (s, 3H), 3.59-3.49 (m, 6H), 3.05 (d, J=10.7 Hz, 2H), 2.98-2.81 (m, 5H), 2.70 (t, J=7.1 Hz, 2H), 2.65-2.55 (m, 3H), 2.46-2.37 (m, 2H), 2.35-2.28 (m, 2H), 2.06-1.97 (m, 1H), 1.84-1.79 (m, 2H), 1.47 (s, 4H), 1.41-1.30 (m, 2H). The compound structure is confirmed.

### Example 90: Synthesis of compound TPD12235

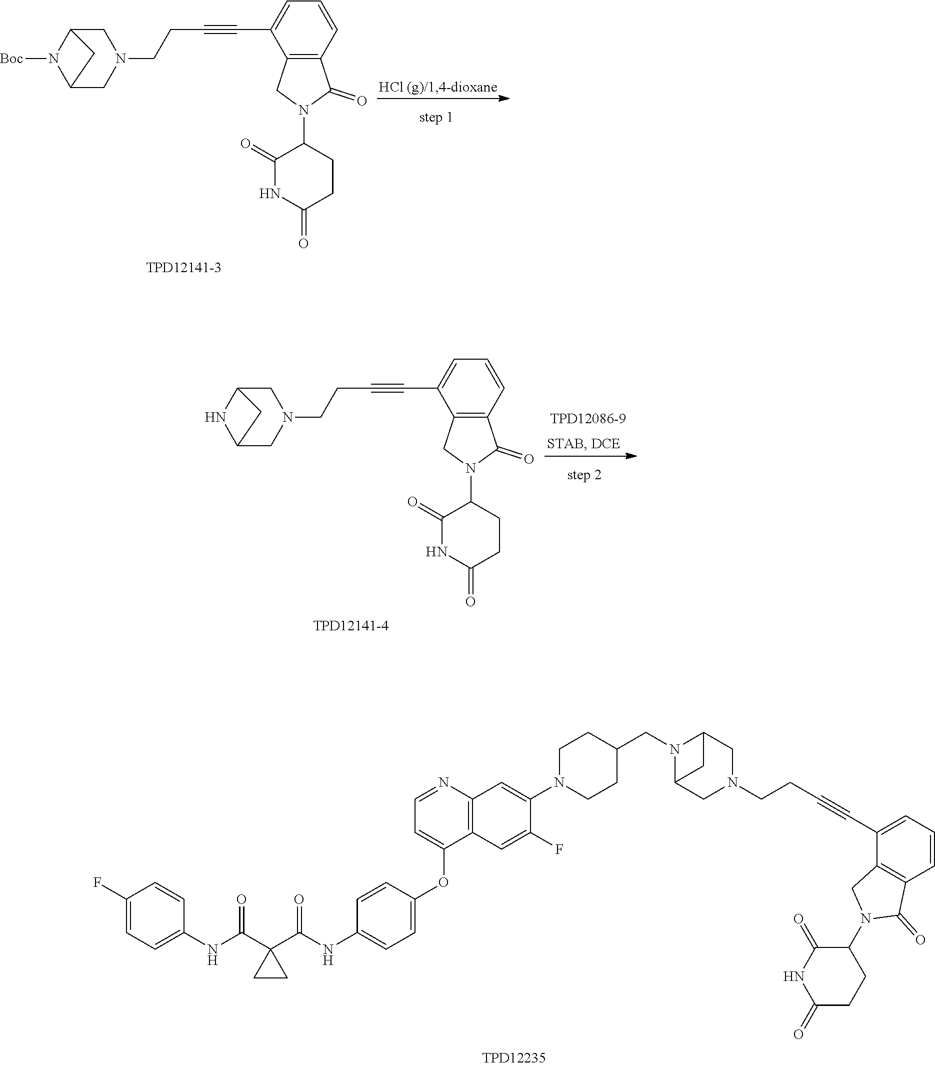 [SMILES: CC(C)(C)OC(=O)N1C2CC1CN(CCC#Cc1cccc3c1CN(C1CCC(=O)NC1=O)C3=O)C2 ; O=C1CCC(N2Cc3c(C#CCCN4CC5CC(C4)N5)cccc3C2=O)C(=O)N1 ; O=C1CCC(N2Cc3c(C#CCCN4CC5CC(C4)N5CC4CCN(c5cc6nccc(Oc7ccc(NC(=O)C8(C(=O)Nc9ccc(F)cc9)CC8)cc7)c6cc5F)CC4)cccc3C2=O)C(=O)N1]

Compound TPD12141-3 (70 mg, 1.14 mmol) 4 N HCl/1,4-dioxane (6 ml) was added sequentially in a 25 ml single-necked vial. The reaction was carried out at 25° C. for 2 h. After completion of the reaction, it was concentrated to dryness to give the crude compound TPD12141-4 (65 mg, yellow solid, purity 85.176%), yield: 99.51%. LCMS (ESI) m/z calcd. for C₂₂H₂₄N₄O₃ [M+H]⁺ 393.18; found 393.1. The compound structure is confirmed.

To a 25 ml single-necked vial were sequentially added compound TPD12141-4 (70 mg, 0.18 mmol), compound TPD12086-9 (102 mg, 0.18 mmol), sodium triacetoxyborohydride (76 mg, 0.36 mmol) and DCE (2 ml). The reaction was carried out under nitrogen protection at 25° C. for 16 hours. After the reaction was completed, the reaction solution was poured into water (3 ml) and an appropriate amount of saturated NaHCO₃ solution was added to make the reaction solution alkaline. It was extracted three times with DCM (15 ml). The organic phases were combined, washed with saturated brine, dried over anhydrous sodium sulfate, filtered, and concentrated to dryness. The residue was prepared by high performance liquid chromatography (HPLC) with the following parameters: column: XBridge-1 5 μm 19-150 mm; mobile phase: acetonitrile-water (0.1% FA); gradient: 10-55/8 min to give the compound TPD12235 (15.8 mg, yellow solid, purity 98.969%) in the following yield: 8.52%. LCMS (ESI) m/z calcd. for C₅₄H₅₂F₂N₈O [M+H]⁺ 947.40; found 948.3; ¹H NMR (400 MHz, DMSO_d₆): δ=11.03 (s, 1H), 10.19 (s, 1H), 10.05 (s, 1H). 8.56 (d, J=5.2 Hz, 1H), 8.20 (s, 2H), 7.85 (d, J=13.4 Hz, 1H), 7.79-7.69 (m, 3H), 7.67-7.58 (m, 3H), 7.53 (t, J=7.6 Hz, 1H), 7.43 (d, J=8.4 Hz, 1H), 7.22 (d, J=8.9 Hz, 2H), 7.15 (t, J=8.9 Hz, 2H), 6.45 (d, J=5.2 Hz, 1H). 5.16 (dd, J=13.3, 5.1 Hz, 1H), 4.46 (d, J=17.7 Hz, 1H), 4.31 (d, J=17.7 Hz, 1H), 3.57-3.49 (m, 4H), 3.08 (d, J=10.8 Hz, 2H), 2.98-2.84 (m, 5H), 2.79-2.67 (m 4H), 2.61 (d, J=17.4 Hz, 1H), 2.46-2.30 (m, 5H), 2.14-1.95 (m, 2H), 1.90-1.80 (m, 3H), 1.58-1.44 (m, 5H), 1.42-1.25 (m, 3H). The compound structure is confirmed.

### Example 91: Synthesis of Compound TPD12236

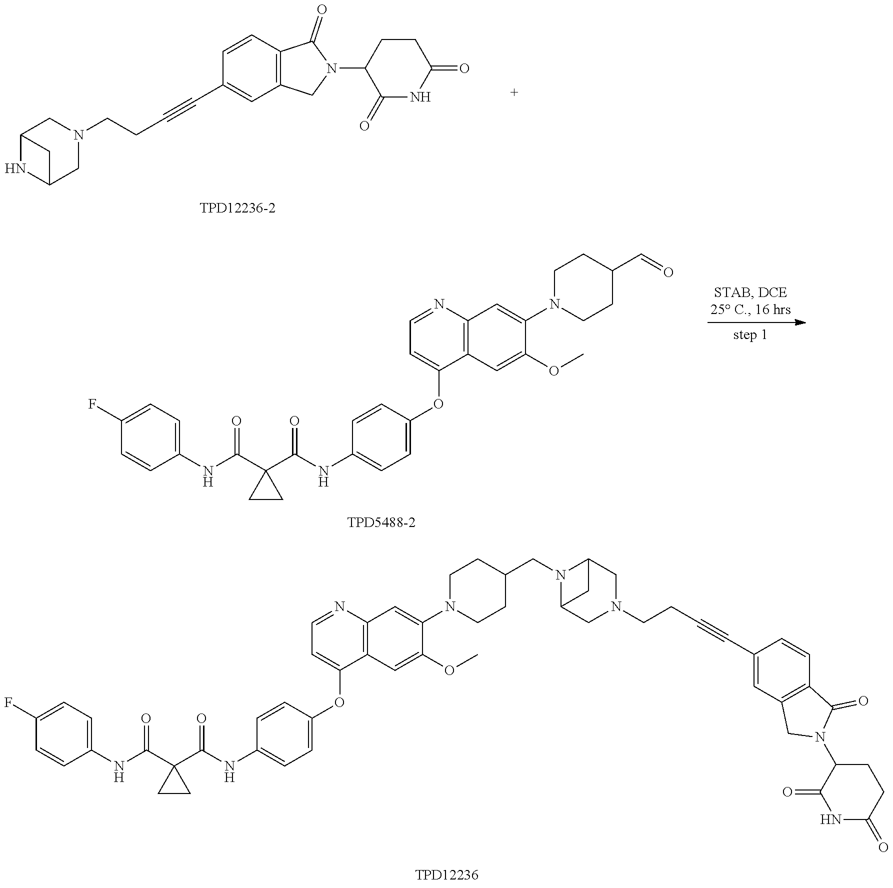 [SMILES: O=C1CCC(N2Cc3cc(C#CCCN4CC5CC(C4)N5)ccc3C2=O)C(=O)N1 ; COc1cc2c(Oc3ccc(NC(=O)C4(C(=O)Nc5ccc(F)cc5)CC4)cc3)ccnc2cc1N1CCC(C=O)CC1 ; COc1cc2c(Oc3ccc(NC(=O)C4(C(=O)Nc5ccc(F)cc5)CC4)cc3)ccnc2cc1N1CCC(CN2C3CC2CN(CCC#Cc2ccc4c(c2)CN(C2CCC(=O)NC2=O)C4=O)C3)CC1]

To a 25 ml single-necked vial was sequentially added compound TPD12236-2 (40 mg, 0.102 mmol), compound TPD5488-2 (59 mg, 0.102 mmol), sodium triacetoxyborohydride (43 mg, 0.204 mmol) and DCE (2 ml). The reaction was carried out under nitrogen protection at 25° C. for 16 hours. After the reaction was completed, the reaction solution was poured into water (6 ml) and an appropriate amount of saturated NaHCO₃ solution was added to make the reaction solution alkaline. It was extracted three times with DCM (5 ml). The organic phases were combined, washed with saturated brine, dried over anhydrous sodium sulfate, filtered, and concentrated to dryness. The residue was prepared by high performance liquid chromatography (HPLC) with the following parameters: column: XBridge-1 5 μm 19-150 mm; mobile phase: acetonitrile-water (0.1% FA); gradient: 10-55/8 min to give the compound TPD12236 (10.67 mg, yellow solid, purity 95.057%) in the following yield: 9.72%. LCMS (ESI) m/z calcd. for C₅₅H₅₅FN₈O₇ [M+H]⁺ 959.09; found 959.4; ¹H NMR (400 MHz, DMSO_d₆): δ=11.03 (s, 1H), 10.18 (s, 1H), 10.06 (s, 1H), 8.43 (d, J=5.2 Hz, 1H), 7.76 (d, J=8.9 Hz, 2H), 7.69 (d, J=7.9 Hz, 1H), 7.64 (s, 3H), 7.51 (d, J=7.9 Hz, 1H), 7.46 (s, 1H), 7.31 (s, 1H), 7.21 (d, J=8.9 Hz, 2H), 7.15 (t, J=8.9 Hz, 2H), 6.39 (d, J=5.1 Hz, 1H), 5.10 (dd, J=13.2, 5.1 Hz, 1H), 4.43 (d, J=17.6 Hz, 1H), 4.31 (d, J=17.5 Hz, 1H), 3.93 (s, 3H), 3.55 (s, 4H), 3.11 (d, J=10.5 Hz, 3H), 2.95-2.80 (m, 5H). 2.72-2.66 (m, 2H), 2.59 (d, J=10.5 Hz, 2H), 2.42-2.28 (m, 5H), 2.00-1.93 (m, 1H), 1.82 (d, J=7.9 Hz, 2H), 1.75 (s, 1H), 1.47 (s, 4H), 1.41-1.31 (m, 2H), 1.23 (s, 1H). The compound structure is confirmed.

### Example 92: Synthesis of Compound TPD12237

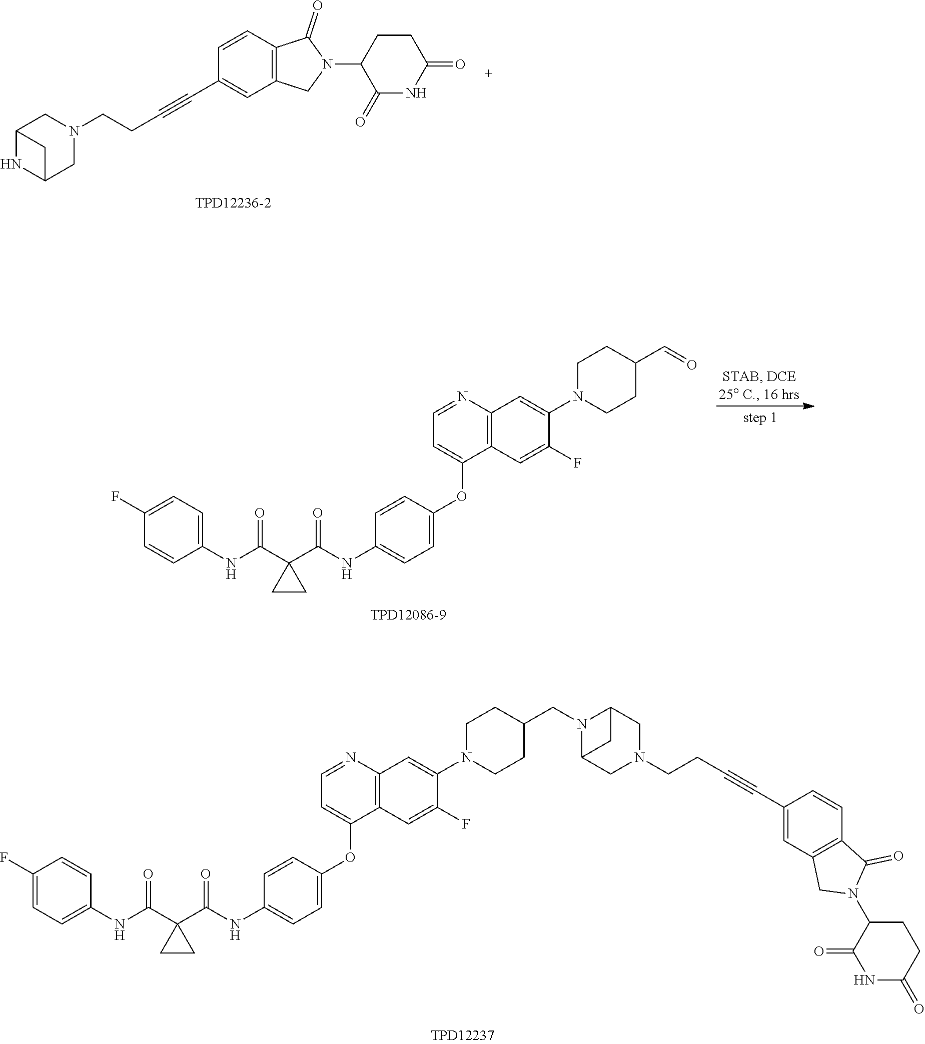 [SMILES: O=C1CCC(N2Cc3cc(C#CCCN4CC5CC(C4)N5)ccc3C2=O)C(=O)N1 ; O=CC1CCN(c2cc3nccc(Oc4ccc(NC(=O)C5(C(=O)Nc6ccc(F)cc6)CC5)cc4)c3cc2F)CC1 ; O=C1CCC(N2Cc3cc(C#CCCN4CC5CC(C4)N5CC4CCN(c5cc6nccc(Oc7ccc(NC(=O)C8(C(=O)Nc9ccc(F)cc9)CC8)cc7)c6cc5F)CC4)ccc3C2=O)C(=O)N1]

To a 25 ml single-necked vial were sequentially added compound TPD12236-2 (40 mg, 0.102 mmol), compound TPD12086-9 (58 mg, 0.102 mmol), sodium triacetoxyborohydride (43 mg, 0.204 mmol) and DCE (2 ml). The reaction was carried out under nitrogen protection at 25° C. for 16 hours. After the reaction was completed, the reaction solution was poured into water (6 ml) and an appropriate amount of saturated NaHCO₃ solution was added to make the reaction solution alkaline. It was extracted three times with DCM (5 ml). The organic phases were combined, washed with saturated brine, dried over anhydrous sodium sulfate, filtered, and concentrated to dryness. The residue was prepared by high performance liquid chromatography (HPLC) with the following parameters: column: XBridge-1 5 μm 19-150 mm; mobile phase: acetonitrile-water (0.1% FA); gradient: 10-55/8 min to give the compound TPD12237 (7.60 mg, yellow solid, purity 99.509%) in the following yield: 7.07%. LCMS (ESI) m/z calcd. for C₅₄H₅₂F₂N₈O [M+H]⁺ 947.4; found 947.4; ¹H NMR (400 MHz, DMSO_d₆): δ=11.03 (s, 1H), 10.19 (s, 1H), 10.06 (s, 1H), 8.56 (d, J=5.2 Hz, 1H), 7.85 (d, J=13.5 Hz, 1H), 7.76 (d, J=8.9 Hz, 2H), 7.69 (d, J=7.9 Hz, 1H), 7.66-7.61 (m, 3H), 7.51 (d, J=8.1 Hz, 1H), 7.43 (d, J=8.5 Hz, 1H), 7.22 (d, J=8.9 Hz, 2H), 7.15 (t, J=8.9 Hz, 2H), 6.45 (d, J=5.2 Hz, 1H), 5.11 (dd, J=13.2, 5.1 Hz, 1H), 4.44 (d, J=17.6 Hz, 1H), 4.32 (d, J=17.5 Hz, 1H), 3.54 (d, J=11.3 Hz, 4H), 3.10 (d, J=10.7 Hz, 3H), 2.94-2.84 (m, 5H), 2.77-2.67 (m, 4H), 2.57 (d, J=17.4 Hz, 1H), 2.38-2.32 (m, 3H), 2.01-1.95 (m, 1H), 1.84 (t, J=11.5 Hz, 3H), 1.75 (s, 1H), 1.47 (s, 4H). 1.42-1.33 (m, 2H). The compound structure is confirmed.

### Example 93: Synthesis of compound TPD12238

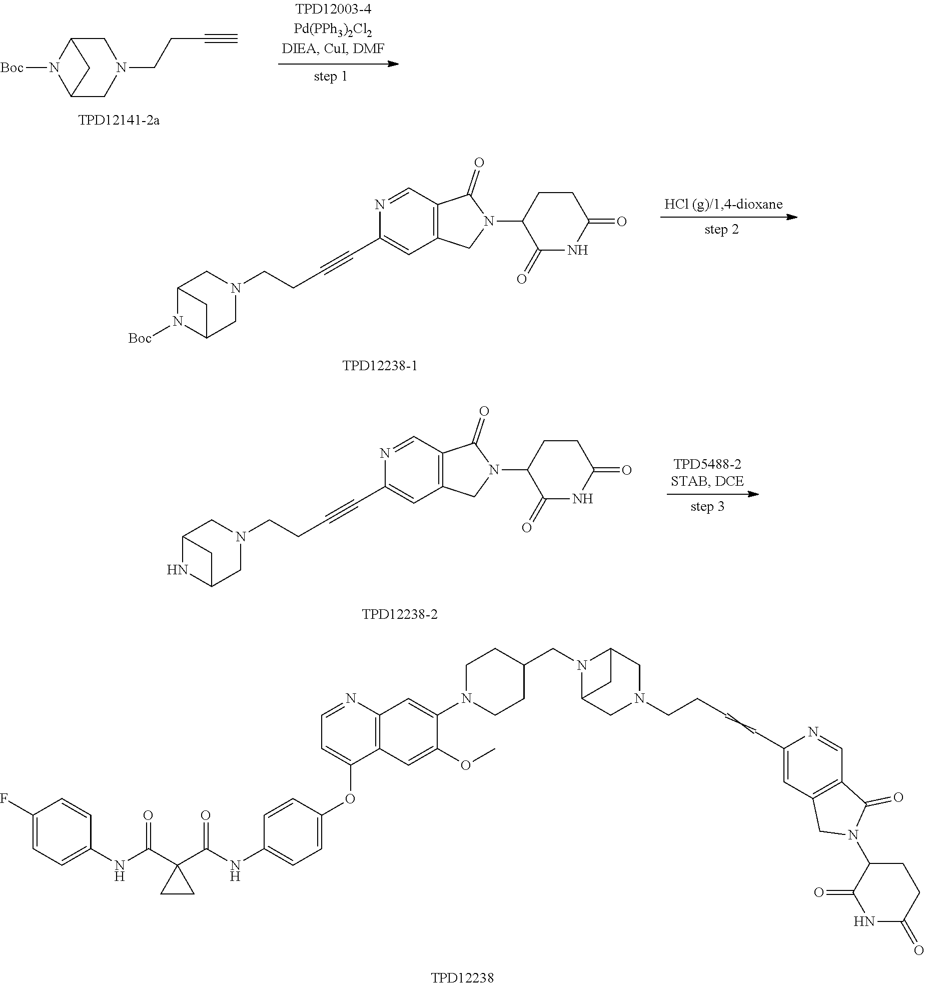 [SMILES: C#CCCN1CC2CC(C1)N2C(=O)OC(C)(C)C ; CC(C)(C)OC(=O)N1C2CC1CN(CCC#Cc1cc3c(cn1)C(=O)N(C1CCC(=O)NC1=O)C3)C2 ; O=C1CCC(N2Cc3cc(C#CCCN4CC5CC(C4)N5)ncc3C2=O)C(=O)N1 ; COc1cc2c(Oc3ccc(NC(=O)C4(C(=O)Nc5ccc(F)cc5)CC4)cc3)ccnc2cc1N1CCC(CN2C3CC2CN(CCC=Cc2cc4c(cn2)C(=O)N(C2CCC(=O)NC2=O)C4)C3)CC1]

To a 50 ml single-necked vial was sequentially added compound TPD12141-2a (1 g, 3.58 mmol), compound TPD12003-4 (1.08 g, 4.29 mmol), tetrakis(triphenylphosphine)palladium (413 mg, 0.36 mmol), cuprous iodide (34 mg, 0.18 mmol), DIEA (924 mg, 7.15 mmol) and DMF (15 ml). The reaction was carried out under nitrogen protection at 80° C. for 16 hours. After completion of the reaction, the reaction solution was reduced to room temperature, decanted into water (60 ml) and extracted three times with EA (15 ml). The organic phases were combined, washed with saturated brine, dried over anhydrous sodium sulfate, filtered, and concentrated to dryness. The residue was purified by silica gel column with eluent ratio of DCM/MeOH=100/1˜30/1 to give compound TPD12238-1 (500 mg, yellow solid, purity 98.367%), yield: 27.87%. LCMS (ESI) m/z calcd. for C₂₆H₃₁N₅O₅ [M+H]⁺ 494.2; found 494.3; ¹H NMR (400 MHz, CDCl₃): δ=9.06 (s, 1H), 8.15 (s, 1H), 7.52 (s, 1H), 5.21 (dd, J=13.3, 5.2 Hz, 1H), 4.54 (d, J=17.1 Hz, 1H), 4.35 (d, J=17.1 Hz, 1H), 4.07 (s, 2H), 3.19 (d, J=65.4 Hz, 2H), 2.93-2.80 (m, 6H), 2.41-2.21 (m, 4H), 1.65 (d, J=6.8 Hz, 2H), 1.45 (s, 9H). The compound structure is confirmed.

To a 25 ml single-necked vial were sequentially added compound TPD12238-1 (150 mg, 0.30 mmol) and 4 N HCl/1,4-dioxane (10 ml). The reaction was carried out at 25° C. for 2 h. After completion of the reaction, it was concentrated to dryness to give the crude compound TPD12238-2 (150 mg, yellow solid, purity 72.993%), yield: 60.406%. LCMS (ESI) m/z calcd. for C₂₁H₂₃N₅O₃ [M+H]⁺ 394.18; found 394.2. It is seen that the compound is structurally correct.

To a 25 ml single-necked vial was sequentially added compound TPD12238-2 (150 mg, 0.38 mol), compound TPD5488-2 (222 mg, 0.38 mmol), sodium triacetoxyborohydride (162 mg, 0.76 mmol) and DCE (2 ml). The reaction was carried out under nitrogen protection at 25° C. for 16 hours. After the reaction was completed, the reaction solution was poured into water (6 ml) and an appropriate amount of saturated NaHCO₃ solution was added to make the reaction solution alkaline. It was extracted three times with DCM (5 ml). The organic phases were combined, washed with saturated brine, dried over anhydrous sodium sulfate, filtered, and concentrated to dryness. The residue was prepared by high performance liquid chromatography (HPLC) with the following parameters: column: XBridge-1 5 μm 19-150 mm; mobile phase: acetonitrile-water (0.1% FA); gradient: 10-55/8 min to give the compound TPD12238 (10.5 mg, yellow solid, purity 82.478%) in the following yield: 2.12%. LCMS (ESI) m/z calcd. for C₅₄H₅₄FN₉O₇ [M+H]⁺ 962.41; found 962.4; ¹H NMR (400 MHz, DMSO_d₆): δ=11.04 (s, 1H), 10.18 (s, 1H), 10.05 (s, 1H), 8.93 (s, 1H), 8.43 (d, J=5.1 Hz, 1H). 7.75 (d, J=8.7 Hz, 2H), 7.68-7.58 (m, 3H), 7.46 (s, 1H), 7.32 (s, 1H), 7.27-7.07 (m, 4H), 6.61 (d, J=11.9 Hz, 1H), 6.39 (d, J=5.2 Hz, 1H), 6.14-5.98 (m, 1H). 5.13 (dd, J=13.4, 5.4 Hz, 1H), 4.52 (t, J=15.9 Hz, 1H), 4.44-4.35 (m, 1H), 3.94 (s, 4H), 3.66-3.52 (m, 5H), 3.04-2.76 (m, 10H), 2.71-2.55 (m, 6H), 2.45-2.22 (m, 3H), 2.09-1.72 (m, 4H), 1.48 (s, 5H), 1.43-1.25 (m, 2H). The compound structure is confirmed.

### Example 94: Synthesis of Compound TPD12239 & Compound TPD12239a

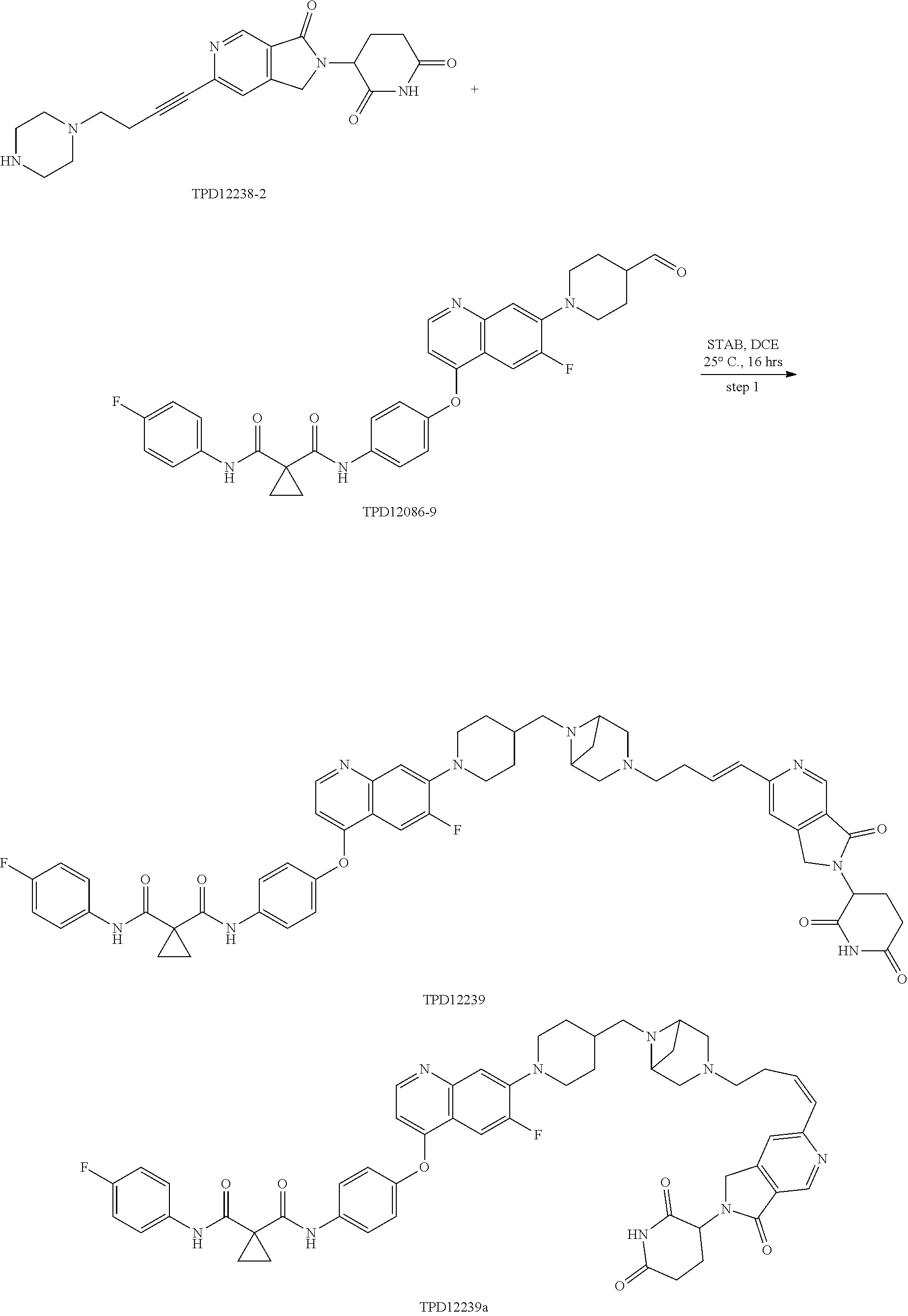

To a 25 ml single-necked vial were sequentially added compound TPD12238-2 (100 mg, 0.25 mmol), compound TPD12086-9 (145 mg, 0.25 mmol), sodium triacetoxyborohydride (108 mg, 0.51 mmol) and DCE (4 ml). The reaction was carried out under nitrogen protection at 25° C. for 16 hours. After the reaction was completed, the reaction solution was poured into water (10 ml) and an appropriate amount of saturated NaHCO₃ solution was added to make the reaction solution alkaline. It was extracted three times with DCM (5 ml). The organic phases were combined, washed with saturated brine, dried over anhydrous sodium sulfate, filtered, and concentrated to dryness. The residue was prepared by high performance liquid chromatography (HPLC) with the following parameters: column: T3 5 μm 19-150 mm; mobile phase: acetonitrile-water (0.1% FA); gradient: 15-35/9 min to give the compound TPD12239 (11.33 mg, white solid, purity 99.700%, time to front: 8.74 min), the compound TPD12239a (1.59 mg, white solid, purity 96.635%, time to peak: 8.51 min).

Compound TPD12239: LCMS (ESI) m/z calcd. for C₅₃H₅₃F₂N₉O₆ [M+H]⁺ 950.4; found 950.4; ¹H NMR (400 MHz, DMSO_d₆): δ=11.04 (s, 1H), 10.19 (s, 1H), 10.05 (s, 1H), 8.93 (s, 1H). 8.56 (d, J=5.2 Hz, 1H), 7.85 (d, J=13.5 Hz, 1H), 7.76 (d, J=8.7 Hz, 2H), 7.69-7.56 (m, 3H), 7.44 (d, J=8.4 Hz, 1H), 7.21 (d, J=8.7 Hz, 2H), 7.15 (t, J=8.8 Hz, 2H) (m, 1H), 6.60 (d, J=11.9 Hz, 1H), 6.45 (d, J=5.1 Hz, 1H), 6.13-6.01 (m, 1H), 5.13 (dd, J=13.2, 5.0 Hz, 1H), 4.54 (d, J=18.3 Hz, 1H), 4.41 (d, J=18.2 Hz, 1H), 3.65-3.50 (m, 4H), 3.03-2.84 (m, 6H), 2.81-2.64 (m, 4H), 2.44-2.27 (m, 5H), 2.02-1.95 (m, 1H), 1.83 (d, J=12.3 Hz, 3H), 1.75 (s, 1H), 1.55 (s, 1H), 1.47 (s, 4H), 1.40-1.32 (m, 2H). The compound structure is confirmed.

Compound TPD12239a: LCMS (ESI) m/z calcd. for C₅₃H₅₃F₂N₉O₆ [M+H]⁺ 951.4; found 951.5; ¹H NMR (400 MHz, DMSO_d₆): δ=11.02 (s, 1H), 10.19 (s, 1H), 10.06 (s, 1H), 8.85 (s, 1H), 8.56 (d, J=5.3 Hz, 1H), 7.85 (d, J=13.4 Hz, 1H), 7.76 (d, J=8.6 Hz, 2H), 7.64 (t, J=6.7 Hz, 3H), 7.43 (d, J=8.5 Hz, 1H), 7.21 (d, J=8.6 Hz, 2H), 7.15 (t, J=8.6 Hz, 2H), 7.07-6.91 (m, 1H), 6.70 (d, J=15.8 Hz, 1H), 6.45 (d, J=5.2 Hz, 1H), 5.11 (dd, J=13.3, 5.0 Hz, 1H), 4.51 (d, J=18.2 Hz, 1H), 4.38 (d, J=18.0 Hz, 1H), 3.51 (dd, J=22.2, 8.0 Hz, 8H), 2.97 (d, J=11.0 Hz, 2H), 2.84-2.72 (m, 5H), 2.34-2.23 (m, 4H), 2.02-1.97 (m, 1H), 1.82 (dd, J=20.1, 9.9 Hz, 3H), 1.75 (s, 1H), 1.47 (s, 5H), 1.38-1.32 (m, 2H). The compound structure is confirmed.

### Example of Tests

### 1. Determination of the Inhibitory Effect of Compounds of the Present Invention on the Growth of Cancer Cells

Inoculate gastric cancer cells (SNU5 or MKN45) into a 96 well plate at a concentration of 4×10 Å cells/ml, with 100 μl added to each well. Incubate for 30 minutes. Observe the cell status under a microscope. Prepare compounds, weigh a certain amount of the series compounds or reference drug cabozantinib of this patent, first prepare a high concentration mother liquor with pure DMSO, and use gradient dilution method to prepare a concentration of 200 μM, 60 μM, 20 μM, 6 μM, 2 μM, 600 nM, 200 nM, 0 nM drug solution, shake to make it fully mixed. The control group was prepared with cabozantinib using the same solvent and method.

After preparation, add 1 μl of drug solution to each well of the 96 well plate, and then add 100 μl of culture medium. Repeat each dose three times. Gently shake to allow the cells and medication to come into full contact and incubate in the incubator for 72 hours. 72 hours later, remove and color with CCK-8 reagent kit. Add 20 μl of CCK-8 reagent to each well, gently shake well, and incubate in the incubator for 1 hour. After 1 hour, remove and measure the absorbance of each well using an enzyme-linked immunosorbent assay (ELISA) reader at a wavelength of 450 nm.

According to the formula: Cell growth inhibition rate=[1−(absorbance of experimental group absorbance of culture medium control group)/(absorbance of blank control group−absorbance of culture medium control group)]×100%. It can be concluded that the compound has the ability to inhibit the growth of gastric cancer cells at the current dose and time.

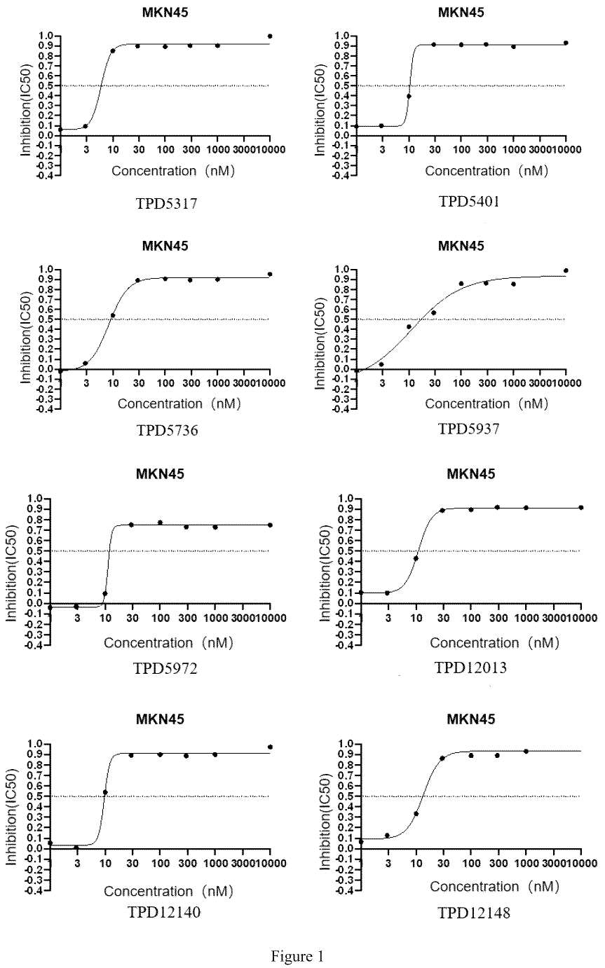

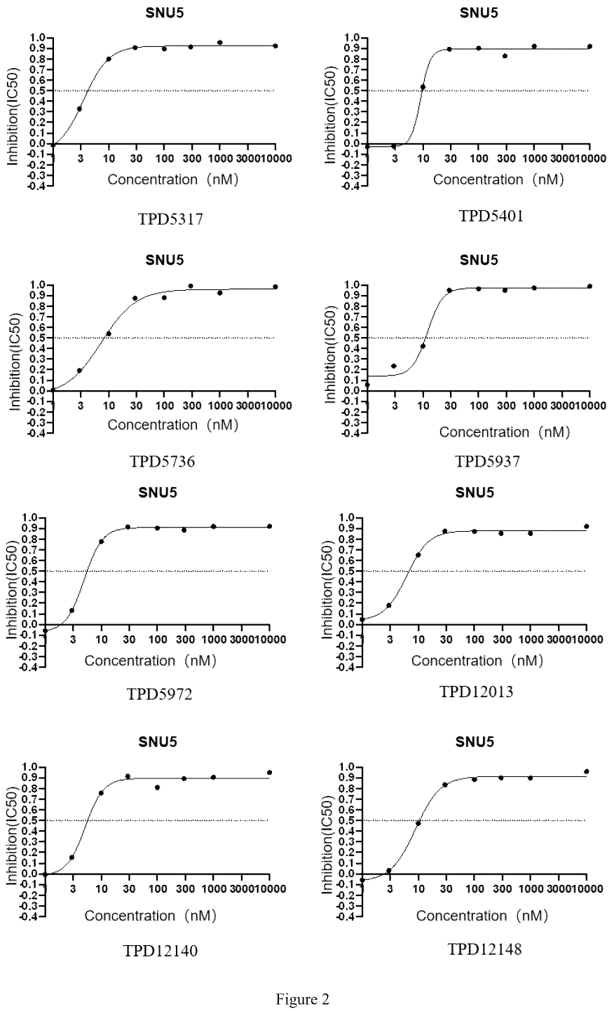

Draw a graph based on the gradient of concentration and use GraphPad Prism 9 software (GraphPad Software, La Jolla, CA) to calculate the maximum inhibitory concentration (IC50) at 50%. The results are shown in Table 2 and FIGS. 1-2. It showed that the compound of the present invention significantly inhibits the proliferation of tumor cells.

### 2. Determination of the Degradation Effect of Compounds of the Present Invention on Target Proteins

Western blot experiment: Take gastric cancer cells (MKN45) and inoculate them into a 6-well plate at a concentration of 2×10 Å cells/ml, adding 2 ml to each well and incubating for 24 hours.

The next day, observe the cells under a microscope to confirm their status. Prepare compounds, weigh a certain amount of the series compounds or reference drug cabozantinib from this patent, first prepare a high concentration mother liquor with pure DMSO, and use gradient dilution method to prepare compound concentrations of 2 mM, 200 μM, 100 μM, 20 μM, 2 μM, 200 nM, and 0 nM. The final concentration of DMSO is 0.5%. Shake to thoroughly mix. Add 10 μl of solution to each well. After incubation for 24 hours, remove the cells and use a pipette to aspirate the suspended cells into a 1.5 ml centrifuge tube. Centrifuge at 5000 rpm for 10 minutes to precipitate the cells. Carefully remove the cell precipitate from the culture medium, add 1 ml of PBS and shake slightly. After repeated cleaning for 3 times, dry the residue. Add 150 μl of RIPA lysate pre mixed with 1% PMSF, blow to allow the lysate to fully contact the cells, and incubate on ice for 10 minutes. Centrifuge at 12000 rpm for 10 minutes, extract the supernatant, use the BCA protein quantification kit to determine and homogenize the sample loading.

Mix BCA-A in a 50:1 ratio Prepare working solution with B solution, dilute the sample by 5 times, then pipette 20 μl onto a 96 well ELISA plate. Add 200 μl of pre mixed BCA working solution and incubate at 37° C. for 20 minutes. Measure the A562 nm absorbance value using an ELISA reader and substitute it into the protein standard curve formula to obtain the sample concentration. After calculating the required volume for loading 20 g of sample, divide the sample into 1.5 ml centrifuge tubes. Add 10 μl of sample buffer to each tube and mix well. Heat in a 95° C. metal bath for 5 minutes to denature the protein.

Take 4-20% gradient pre adhesive and fix it on the adhesive rack. Add electrophoresis solution to the slot and remove the comb, Marker and protein samples were loaded, and after 30 minutes of electrophoresis at 80V, the voltage was adjusted to 120V for electrophoresis, The marker band stops electrophoresis at the forefront of the gel. Take out the adhesive block and cut it to the appropriate size, The PVDF membrane was cut and activated in methanol, and after activation, the PVDF membrane was equilibrated in a membrane transfer equilibrium solution for 1 minute. Using sponge pads The sequence of PVDF film, adhesive, and sponge pad is placed in the splint and placed in the eBlot equipment for rapid wet rotation. Remove the membrane and seal it in 5% skim milk powder dissolved in 1×TBST for 1 hour. Cut the membrane according to the molecular weight of the target protein and internal reference protein, and place it in primary antibody diluted with 1×TBST. Shake overnight at 4° C.

The next day, transfer the band to 1×TBST for 3 washes, then add a specific dilution of the secondary antibody and shake it for 1.5 hours. Transfer the band again to 1×TBST for 3 washes, prepare ECL color solution, place the membrane in the imaging system, cover the surface with ECL color solution, and take photos of the membrane using the imaging system.

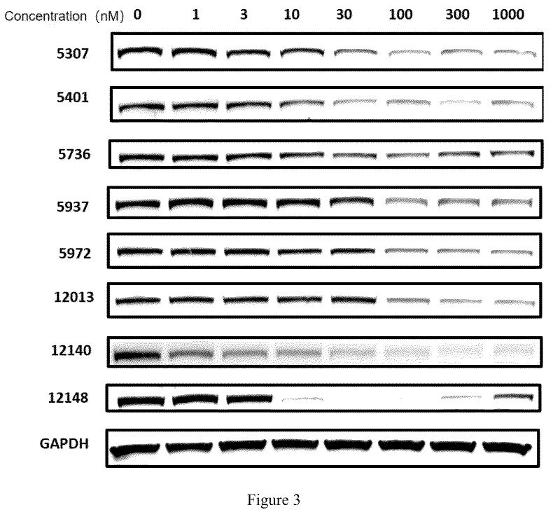

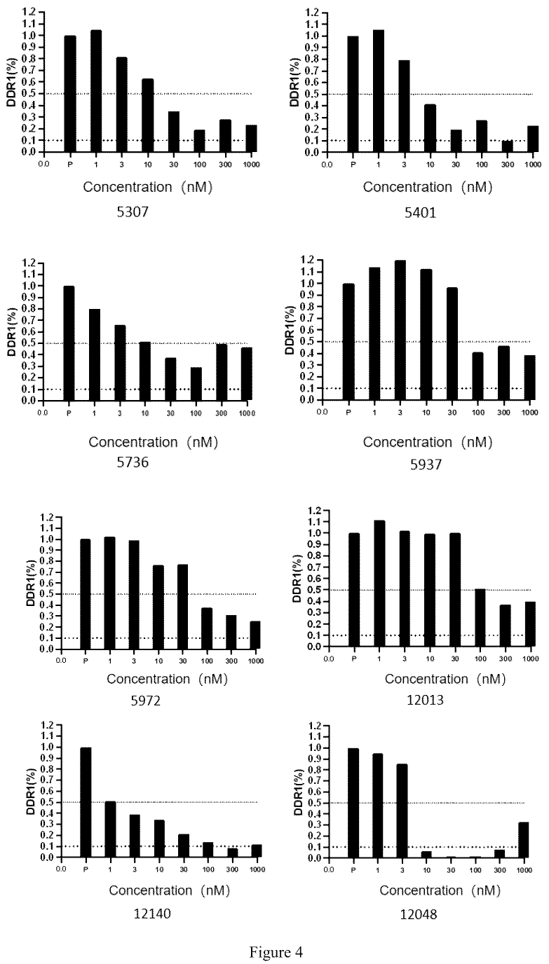

Using the SHST analysis system to analyze the results, measure the grayscale values of each band of the target protein and internal reference protein. Normalize the grayscale values of the target protein/corresponding lane GAPDH grayscale values, and perform quantitative analysis according to the protein amount=experimental group grayscale value/blank control group grayscale value. The results are shown in Table 2 and FIGS. 3-4 below.

It showed that the compound of the present invention significantly degrades the target protein DDR1 in tumor cells.

### 3. The Growth Inhibition Effect of the Compound of the Present Invention on Cancer Cells and the Degradation Effect of Target Proteins

Referring to steps 1 and 2 of this embodiment, the inhibitory effect of the compound of the present invention on the growth of cancer cells and the degradation effect of target proteins were measured, and the following results were obtained (see Table 2 below).

The growth inhibitory effects of the compound of the present invention on tumor cell lines MKN45 and SNU5 were measured. The activity is expressed as half inhibitory concentration (C50). The results are shown in Table 2 below; When the half inhibitory concentration is less than or equal to 100 nM, the compound activity is labeled as “++++”; When the half inhibitory concentration is greater than 100 nM and less than 500 nM, the compound activity is labeled as “++”; When the half inhibitory concentration is greater than 500 nM and less than 1000 nM, the compound activity is labeled as “++”; When the half inhibitory concentration is greater than 1000 nM and less than 3000 nM, the compound activity is marked as “+”; When the half inhibitory concentration is greater than 3000 nM, the compound activity is marked as

The activity of the compound of the present invention in degrading DDR in tumor cell line MKN45 was measured. The activity is expressed as half degradation concentration (DC50) and maximum degradation percentage (Dmax); The results are shown in Table 2 below; When the half degradation concentration is less than or equal to 1 nM, the compound activity is marked as “++++”; When the half degradation concentration is greater than 100 nM and less than 500 nM, the compound activity is marked as “++”; When the half degradation concentration is greater than 500 nM and less than 1000 nM, the compound activity is marked as “++”; When the half degradation concentration is greater than 1000 nM and less than 3000 nM, the compound activity is marked as “+”; When the half degradation concentration is greater than 3000 nM, the compound activity is marked as “-”. When the maximum degradation percentage is greater than or equal to 85%, the compound activity is marked as “++++”; When the maximum degradation percentage is greater than 75% and less than 85%, the compound activity is marked as “++”; When the maximum degradation percentage is greater than 65% and less than 75, the compound activity is marked as “++”; When the maximum degradation percentage is greater than 50% and less than 65%, the compound activity is marked as “+”; When the maximum degradation percentage is less than 50%, the compound activity is marked as “-”

TABLE 2
Growth inhibition of cancer cells and target protein degradation
by different compounds of the present invention

| Chemical | IC50- | IC50- | DC50- | Dmax- |
|---|---|---|---|---|
| compounds | MKN45 | SNU5 | DDR1 | DDR1 |
| compound | +++ | +++ | ++ | ++ |
| TPD5305 | | | | |
| compound | +++ | +++ | ++++ | ++ |
| TPD5313 | | | | |
| compound | +++ | +++ | ++++ | ++++ |
| TPD5315 | | | | |
| compound | ++ | ++ | ++++ | +++ |
| TPD5316 | | | | |
| compound | +++ | +++ | ++ | ++ |
| TPD5317 | | | | |
| compound | ++++ | ++++ | + | ++ |
| TPD5401 | | | | |
| compound | ++++ | ++++ | +++ | ++ |
| TPD5402b | | | | |
| compound | +++ | +++ | +++ | +++ |
| TPD5406 | | | | |
| compound | +++ | ++ | ++++ | +++ |
| TPD5407 | | | | |
| compound | +++ | +++ | +++ | ++ |
| TPD5409 | | | | |
| compound TPD5410 | ++++ | ++++ | + | ++ |
| compound TPD5411 | +++ | +++ | +++ | +++ |
| compound TPD5412 | +++ | +++ | + | ++ |
| compound TPD5415 | +++ | +++ | ++ | ++ |
| compound TPD5417 | ++ | + | ++ | ++ |
| compound TPD5420 | + | ++ | +++ | ++ |
| compound TPD5421 | +++ | +++ | +++ | +++ |
| compound TPD5423 | +++ | +++ | ++ | ++ |
| compound TPD5424 | + | ++ | + | ++ |
| compound TPD5430 | + | + | +++ | ++ |
| compound TPD5431 | + | + | +++ | ++ |
| compound TPD5432 | +++ | ++++ | ++++ | +++ |
| compound TPD5436 | +++ | +++ | +++ | ++ |
| compound TPD5452 | + | + | + | ++ |
| compound TPD5453 | + | ++ | +++ | +++ |
| compound TPD5457 | ++++ | ++++ | ++++ | +++ |
| compound TPD5488 | ++++ | ++++ | ++++ | +++ |
| compound TPD5494 | + | +++ | ++++ | ++++ |
| compound TPD5495 | +++ | +++ | ++++ | +++ |
| compound TPD5496 | + | + | ++++ | ++++ |
| compound TPD5505 | + | ++ | ++++ | +++ |
| compound TPD5706 | +++ | +++ | +++ | +++ |
| compound TPD5714 | +++ | ++++ | +++ | ++ |
| compound TPD5734 | +++ | +++ | ++++ | ++ |
| compound TPD5735 | ++ | +++ | ++++ | +++ |
| compound TPD5736 | ++++ | +++ | ++++ | +++ |
| compound TPD5745 | ++ | ++ | + | ++ |
| compound TPD5746 | +++ | ++ | +++ | +++ |
| compound | +++ | +++ | ++++ | ++++ |
| TPD5752m | | | | |
| compound TPD5760 | +++ | +++ | ++ | ++ |
| compound TPD5773 | ++ | +++ | ++++ | ++ |
| compound TPD5774 | +++ | ++ | ++++ | ++++ |
| compound TPD5901 | ++++ | ++++ | ++++ | ++++ |
| compound TPD5907 | ++++ | ++++ | ++++ | ++++ |
| compound TPD5937 | ++++ | ++++ | ++++ | ++ |
| compound TPD5972 | ++++ | ++++ | ++++ | ++++ |
| compound TPD7022 | +++ | +++ | +++ | ++ |
| compound TPD7026 | ++++ | ++++ | ++++ | ++ |
| compound TPD9004 | +++ | ++++ | ++++ | +++ |
| compound TPD9006 | ++++ | +++ | ++++ | +++ |
| compound | ++ | +++ | + | ++ |
| TPD10001 | | | | |
| compound | + | ++ | + | ++ |
| TPD10013 | | | | |
| compound | ++++ | ++++ | +++ | +++ |
| TPD10040 | | | | |
| compound | ++ | ++++ | ++++ | ++++ |
| TPD10041 | | | | |
| compound | ++ | +++ | ++++ | +++ |
| TPD10050 | | | | |
| compound | ++++ | ++ | ++++ | ++++ |
| TPD10051 | | | | |
| compound | ++++ | ++++ | ++++ | ++ |
| TPD12001 | | | | |
| compound | ++++ | ++++ | ++++ | +++ |
| TPD12003 | | | | |
| compound | ++++ | ++++ | +++ | ++ |
| TPD12009 | | | | |
| ompound | +++ | +++ | ++++ | ++++ |
| TPD12013 | | | | |
| compound | ++++ | ++++ | ++++ | ++ |
| TPD12068 | | | | |
| compound | ++++ | ++++ | ++++ | +++ |
| TPD12081 | | | | |
| compound | +++ | ++++ | ++++ | ++ |
| TPD12082 | | | | |
| compound | ++++ | ++++ | ++++ | ++ |
| TPD12083 | | | | |
| compound | +++ | ++++ | ++++ | +++ |
| TPD12086 | | | | |
| compound | +++ | ++++ | +++ | ++ |
| TPD12088 | | | | |
| compound | ++++ | ++++ | ++++ | ++ |
| TPD12096 | | | | |
| compound | ++++ | ++++ | ++++ | ++ |
| TPD12097 | | | | |
| compound | ++++ | ++++ | ++++ | ++ |
| TPD12100 | | | | |
| compound | ++++ | ++++ | ++++ | ++++ |
| TPD12101 | | | | |
| compound | ++++ | ++++ | ++++ | +++ |
| TPD12102 | | | | |
| compound | ++++ | ++++ | ++++ | +++ |
| TPD12110 | | | | |
| compound | ++++ | ++++ | ++++ | ++ |
| TPD12112 | | | | |
| compound | ++++ | ++++ | ++++ | ++ |
| TPD12113 | | | | |
| compound | ++++ | ++++ | ++++ | +++ |
| TPD12116 | | | | |
| compound | ++++ | ++++ | ++++ | ++ |
| TPD12118 | | | | |
| compound | ++++ | ++++ | ++++ | +++ |
| TPD12139 | | | | |
| compound | ++++ | ++++ | ++++ | ++ |
| TPD12140 | | | | |
| compound | ++++ | ++++ | ++++ | ++ |
| TPD12148 | | | | |
| compound | ++++ | ++++ | ++++ | +++ |
| TPD12149 | | | | |
| compound | ++++ | +++ | +++ | ++ |
| TPD12150 | | | | |
| compound | ++++ | ++++ | ++++ | ++ |
| TPD12162 | | | | |
| compound | ++++ | ++++ | ++++ | ++ |
| TPD12163 | | | | |
| compound | ++++ | ++++ | ++++ | ++ |
| TPD12205 | | | | |
| compound | ++++ | ++++ | ++++ | +++ |
| TPD12215 | | | | |
| compound | ++ | ++++ | ++++ | ++++ |
| TPD12216 | | | | |
| compound TPD12218 | ++++ | ++++ | ++++ | ++ |
| compound TPD12234 | ++++ | ++++ | ++++ | +++ |
| compound TPD12235 | ++++ | ++++ | ++++ | ++ |
| compound TPD12236 | ++++ | ++++ | ++++ | +++ |
| compound TPD12237 | ++++ | ++++ | +++ | ++ |
| compound TPD12238 | ++++ | ++++ | ++++ | ++ |
| compound TPD12239 | ++++ | ++++ | ++++ | +++ |
| compound TPD12239a | ++++ | ++++ | ++++ | ++ |
| cabozantinib control | ++++ | ++++ | − | − |

It showed that the growth inhibitory activity of the compounds of the present invention on the tumor cell lines MKN45 and SNU5 was in the range of 1-3000 nM expressed as half inhibitory concentration (IC50). The degradation activity of the compounds against DDR1 in the MKN45 cell line was in the range of 1-3000 nM expressed as half degradation concentration (DC50); the maximum percentage of degradation was in the range of 50%-95%.

### 4. Pharmacodynamic Study of the Compound of the Present Invention on a Subcutaneous Transplant Tumor Model of Human Gastric Cancer MKN45 Cells in Nude Mice

Using healthy male BALB/c Nude mice as a model, human gastric cancer MKN45 cells were subcutaneously implanted. After 7 days of feeding, the tumor volume reached 75-100 mm3. Six mice were randomly divided into 5 groups: blank group, positive drug control group, drug 10 mg/kg group, drug 30 mg/kg group, and drug 50 mg/kg group.

Precisely weigh the subject compound TPD12140 or positive control drug cabozantinib, dissolve the samples in 5% DMSO/10% PEG400/10% Tween-80/75% Saline, dilute to a low concentration in a gradient, store at 4° C. and reserve for use, and dispense once a week. The drug was administered once a day for 14 consecutive days. Tumor volume was measured, and mice were weighed every two days, and the results are shown in A-B in Supplementary FIG. 5, respectively.

As can be seen, in vivo experiments showed that the drug of the present invention had a significant inhibitory effect on tumors in all dose groups and there was no significant change in the average body weight.

### 5. Detection of DDR1mRNA

Tumor cells were inoculated into 6-well plates with 1-3×10⁵ cells per well, and the cell growth status was observed after 24 hours. After the cells grew to about 80% confluence, compound TPD12140 (working concentration 100 nM) was added to the cell culture medium in the experimental group, and an equal volume of DMSO was added to the control group, and the incubation was continued for 24 hours.

RNA extraction: Discard the culture medium from the well plate, add 1 mL Trizol, and let it sit at room temperature for 10 minutes; Blow the cells several times with a gun, transfer the lysate to a 1.5 ml EP tube treated with DEPC, add 200 μl of chloroform, and shake well for 15 seconds. After standing at room temperature for 2-3 minutes, centrifuge at 4° C. and 12000 rpm for 15 minutes. Then take the upper colorless aqueous phase into the EP tube treated with DEPC, being careful not to suck into the middle protein layer. Add 600 μl of isopropanol and let it stand at room temperature for 10 minutes or −80° C. for 60 minutes. 4° C., 12000 rpm, centrifuge for 15 minutes. Discard the supernatant, add 1.0 ml of 75% ethanol freshly prepared with DEPC water, mix upside down, centrifuge at 4° C. 7500 rpm for 5 minutes, and repeat the above operation once. Abandon the upper clean and use a small gun to suck up the liquid. Dry the precipitate in the air, add DEPC water, mix well to dissolve RNA. Detect the OD 260/280 value of RNA samples. Store the RNA sample in a −80° C. freezer.
cDNA first strand synthesis: Take 500 ng-5 μg of total RNA, add 1 μl of Oligo (dT) Primer (0.5 μg/μL), fill the volume with DEPC water to 12 μl, 65° C. for 5 minutes, and immediately place on ice for 2-5 minutes. Add 4 μl of 5× Reaction Buffer, DNTP (10 mmol/μL) 2 μl, RNAase Inhibitor (20 U/μL) 1 μL, M-MLV (200 U/μL) 1 μl, the reaction system was thoroughly mixed and reacted at 42° C. for 60 minutes, followed by treatment at 70° C. for 15 minutes to inactivate reverse transcriptase, Store cDNA products at −20° C.

Fluorescence quantitative PCR: Using the Invitrogen fluorescence quantitative reagent kit Platinum SYBR green qPCR Super Mix UDG, each sample is equipped with 3 complex wells for each primer pair, and a 20 μl system can be used for each well. After PCR is completed, running the corresponding program can test the reliability of PCR amplification. The fusion curve detection demonstrated the specificity of the amplified target gene and internal reference gene (GAPDH) PCR product. Finally, use the 2-ΔΔΔCt algorithm to perform relative quantitative analysis on the results. The 20 μl PCR amplification reaction system and reaction conditions are: 1 μl of the first strand of cDNA diluted 4 times, 10 μl of 2×SYBR green qPCR Super Mix UDG, 0.5 μl of upstream and downstream primers, and supplementation of dH2O to 20 μl. Reaction conditions: 50° C. for 2 minutes, 95° C. for 2 minutes, 95° C. for 15 seconds, 60° C. for 30 seconds, 72° C. for 30 seconds, 40 cycles. The primer sequences for fluorescence quantitative PCR are as follows:

DDR1-F:
CCGACTGGGTTCGCTTCTACC;
DDR1-R:
CGGTGTAAGACAGGAGTCCATC;
GAPDH-F:
ATCTTCCAGGAGCGAGATCCC;
GAPDH-R:
TGAGTCCTTCCACGATACCAA.

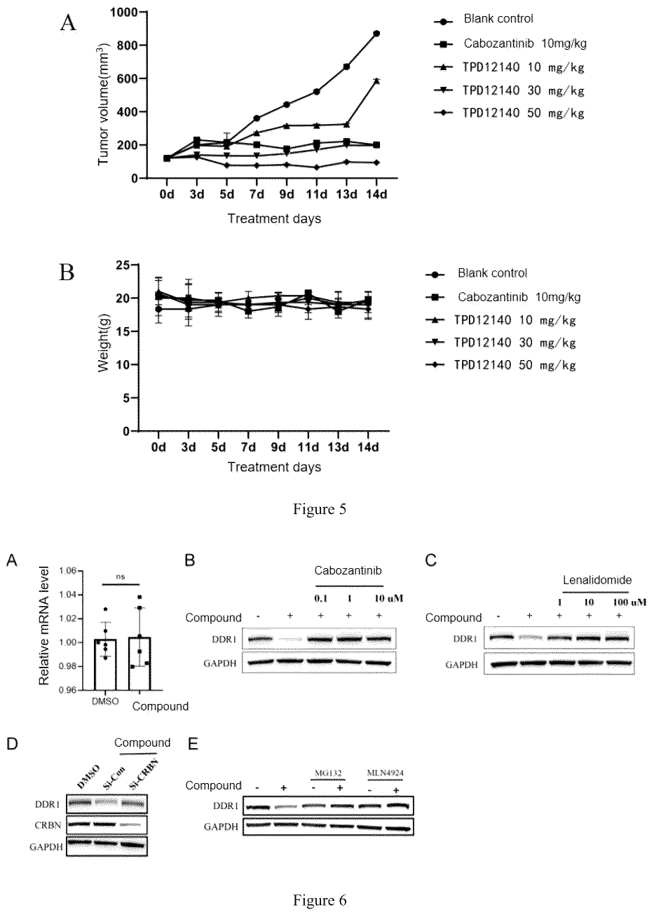

The experiment was repeated three times and more, and the experimental data were statistically analyzed using GraphPad Prism software. The results are shown in FIG. 6, A. As can be seen, the compounds described in the present invention do not affect the expression of DDR1mRNA.

### 6. Competition Inhibition Experiments

Tumor cells were taken and inoculated into 6-well plates with 1×3×10⁵ cells per well, and the cell growth status was observed on the next day. When the cells reached 80% confluence, TPD12140 (working concentration 100 nM) and different concentrations of cabozantinib or lenalidomide were added to the cell culture medium of the experimental group, and the control group was added with an equal volume of DMSO, and the incubation was continued for 24 h. Western Blot was performed to detect the expression of DDR1 protein (the same operation as above). The experiments were repeated three times or more, and the experimental data were statistically analyzed using GraphPad Prism software.

The experimental results are shown as B-C in FIG. 6, which shows that cabozantinib competitively binds to the target protein DDR1 and inhibits the degradation of the compound (B), and lenalidomide competitively binds to the E3 ubiquitin ligase CRBN and inhibits the degradation of DDR1 by the compound (C).

7. siRNA Knockdown Assay

Tumor cells were inoculated into 6-well plates with 1×3×10⁵ cells per well, and cell growth was observed the next day. Transfection was performed when the cell density was about 30-50%. Add CRBN siRNA in a certain volume of antibiotic-free and serum-free medium and mix well, mix the transfection reagent with a certain volume of serum-free and antibiotic-free medium, mix well and leave it at room temperature for 5 min, mix the diluted siRNA and the transfection reagent, mix gently, and leave it at room temperature for 20 min. add the complex slowly into the cell culture medium and mix well by shaking gently. After incubation for 12 h, the cells were replaced with medium and incubated at 37° C. in a C02 incubator for 36 h. TPD12140 (working concentration 100 nM) was added to the cell culture medium of the experimental group, and an equal volume of DMSO was added to the control group, and the cells were further incubated for 24 h. Western Blot was performed to detect the expression of DDR1 protein (same as the above procedure). The experiments were repeated three times and more, and the experimental data were statistically analyzed using GraphPad Prism software.

The experimental results are shown in FIG. 6D. It showed that SiRNA knockdown of the expression of E3 ubiquitin ligase CRBN inhibits the degrading effect of compounds on DDR1.

8. Experiments with Inhibitors of the Ubiquitin-Proteasome System

Tumor cells were inoculated into 6-well plates with 1-3×10⁵ cells per well, and the cell growth status was observed the next day. When the cells grew to about 80% confluence, the proteasome inhibitor MG132 (working concentration 10 uM) or the protein ubiquitination inhibitor MLN4924 (working concentration 1 uM) was added to the cell culture medium of the experimental group, and an equal volume of DMSO was added to the control group, and the incubation was continued for 6 hours. The medium in the well plates was discarded, washed twice with PBS, fresh medium was added, and compound TPD12140 (working concentration 100 nM) or an equal volume of DMSO was added to each group of cells, and the incubation was continued for 18 h. Western Blot was performed to detect the expression of DDR1 protein (same operation as above). The experiments were repeated three times or more, and the experimental data were statistically analyzed using GraphPad Prism software.

The experimental results are shown as E in FIG. 6, which shows that the proteasome inhibitor MG132 and the protein ubiquitination inhibitor MLN4924 can inhibit the degradation of the compounds. The above results indicate that the compounds described herein degrade the DDR1 protein at the protein level through the ubiquitin-proteasome system, and that the degradation is dependent on the formation of the DDR1-compound-E3 ubiquitin ligase ternary complex.

Obviously, the above embodiments are merely examples for the purpose of clear illustration and are not a limitation of the embodiments. For those of ordinary skill in the art, other variations or changes in different forms may be made based on the above description. It is neither necessary nor possible to exhaust all the embodiments herein. The obvious variations or changes derived therefrom remain within the scope of protection of the invention.

1. A bifunctional compound, wherein the compound has a structure as shown in X-L-Y, and an X part, an L part, and a Y part are connected through covalent bonds, wherein
the X part is a ligand capable of binding to receptor tyrosine kinase; preferably, the X part is a ligand capable of binding to discoidin domain receptors (DDRs); more preferably, the X part is a ligand capable of binding to a DDR1 in a discoidin domain receptor family;
the Y part is a ligand capable of binding to E3 ubiquitin ligase; preferably, the Y part is a ligand capable of binding to E3 ligase Cereblon; more preferably, the Y part is a ligand capable of binding to a Von Hippel-Lindau tumor inhibiting factor; and
the L part is a connecting group that connects the X part with the Y part.

2. The bifunctional compound of claim 1, wherein the X part is selected from following structures as shown in X1 to X6, wherein a zigzag line represents a position where the X part is connected with the L part through covalent bonds; 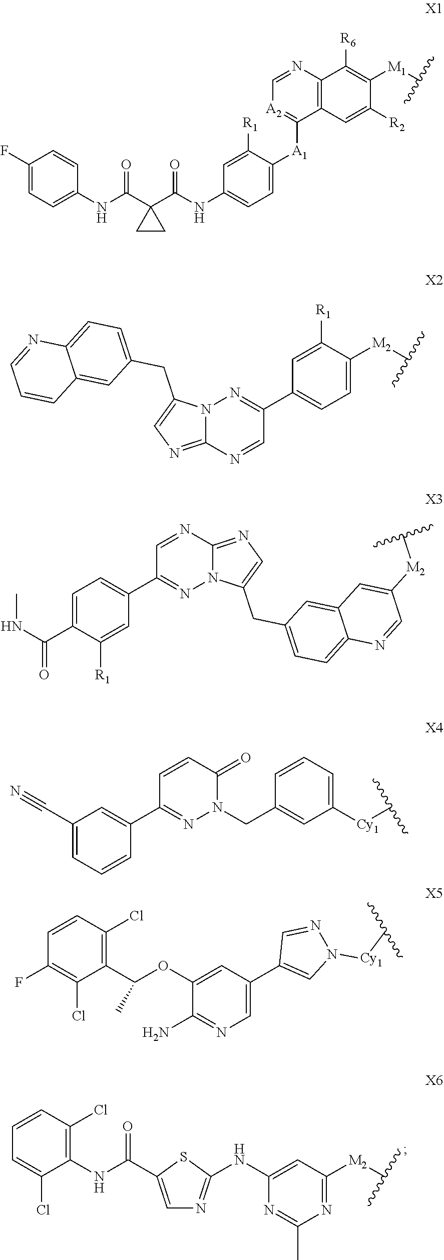
wherein
the R₁ is selected from H, F, or Cl;
the R₂ is selected from H, —OR₃, —NHR₃, F, Cl, or Br;
preferably, in the —NHR₃, the R₃ is selected from H, a C1-4 linear or branched alkyl, or a C1-4 linear or branched alkyl with any hydrogen atom being substituted by fluorine, or ArCO—, wherein, the Ar is selected from H, halogen, a C1-4 linear or branched alkyl, a C1-4 linear or branched alkoxy, or a C1-4 linear or branched amino substituted 5-8-membered aromatic ring group;
preferably, the Ar is preferably H, halogen, a C1-4 linear or branched alkyl, a C1-4 linear or branched alkoxy, or a C1-4 linear or branched amino substituted phenyl;
the R₆ is selected from H, F, Cl, OR₇ or NHR₇;
preferably, in the NHR₇, the R₇ is selected from H or a C1-4 linear or branched alkyl;
the A₁ is selected from O or NH;
the A₂ is selected from N or CH;
the M₁ is selected from NR₃, CH₂, O, C or Cy₁;
the M₂ is selected from NH, O, CO or Cy₁;
the Cy₁ is selected from a substituted or unsubstituted 4-7-membered carbon heteromonocyclic ring, a substituted or unsubstituted 5-10-membered carbon heterofused ring, a substituted or unsubstituted 4-7-membered monocyclic alkyl, a substituted or unsubstituted 5-10-membered cycloalkyl, a substituted or unsubstituted 7-10-membered bridged cycloalkyl, or a substituted or unsubstituted 5-8-membered aromatic ring group;
preferably, in Cy₁, the substitution comprises substitution optionally by 0 to 3 substituents selected from H, F, Cl, OH, COOH, CN, NH₂, a carbonyl, a C1-4 linear or branched alkyl, a halogen-substituted C1-4 linear or branched alkyl, a hydroxyl-substituted C1-4 linear or branched alkyl or a C1-4 alkoxy;
preferably, in the Cy₁, a heteromonocyclic ring and a heterofused ring contain 0 to 4 heteroatoms selected from O, S and N; and the aromatic ring group contains 0 to 3 heteroatoms selected from N, S and O;
more preferably, the Cy₁ is selected from pyridine, piperazine, pyrimidine, pyrazine, pyridazine, benzene, pyrazole, imidazole, triazole, 2,2-difluoropiperidine, 2,2-difluoropiperazine, 2,2,3,3-tetrafluoropiperazine, 2,2,5,5-tetrafluoropiperazine or 2,2,6,6-tetrafluoropiperazine.

3. The bifunctional compound of claim 1, wherein the Y part is a VHL ligand, and the Y part is selected from following structures as shown in Y1 to Y3, wherein an asterisk (x) position represents a position where the Y part is connected with the L part through covalent bonds; 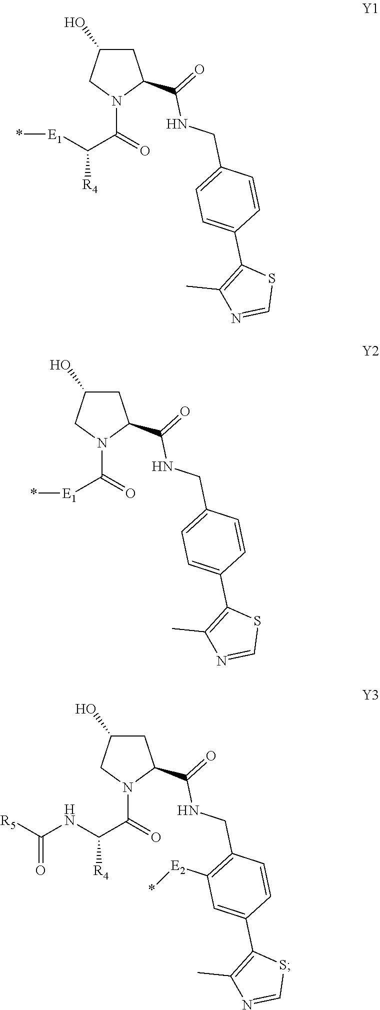
wherein,
the R₄ is selected from H, a C1-C5 linear or branched alkyl, a C3-C6 cycloalkyl, and a 3-6 membered heterocycloalkyl;
preferably, the heterocycloalkyl contains 1 to 3 heteroatoms selected from O, N, and S;
preferably, the R₄ is selected from an isopropyl, a tert butyl, a cyclohexyl or a tetrahydropyranyl;
the R₅ is selected from a C1-C5 linear or branched alkyl, and a C3-C6 substituted cycloalkyl;
preferably, the R₅ is selected from a methyl, an ethyl, an isopropyl, or a 1-fluorocyclopropanyl;
the E₁ is selected from NH, NR₃, CH₂ or Cy₂;
preferably, the Cy₂ is selected from a substituted or unsubstituted 4-7-membered carbon heteromonocyclic ring or a substituted or unsubstituted 5-8-membered aromatic ring group;
preferably, the aromatic ring group and the carbon heteromonocyclic ring are substituted by 0 to 3 substituents selected from H, F, Cl, OH, COOH, CN, NH₂, a carbonyl, a C1-4 linear or branched alkyl, a halogen-substituted C1-4 linear or branched alkyl, a hydroxyl-substituted C1-4 linear or branched alkyl or a C1-4 alkoxy;
preferably, the heteromonocyclic ring contains 0 to 4 heteroatoms selected from O, S and N;
and the aromatic ring group contains 0 to 3 heteroatoms selected from N, S and O;
preferably, the Cy₂ is selected from piperidine, piperazine, azetidine, or 1,2,3-triazole;
the E₂ is selected from O, NH or Cy₃;
preferably, the Cy₃ is selected from a 4-7-membered carbon heteromonocyclic ring;
preferably, the Cy₃ is selected from piperidine, piperazine, or azetidine.

4. The bifunctional compound of claim 1, wherein the Y part is a ligand of E3 ligase Cereblon, and the Y part is selected from a piperidine-2,6-diketone compound, thalidomide or its derivatives, lenalidomide or its derivatives, or pomalidomide or its derivatives.

5. The bifunctional compound of claim 4, wherein the Y part has following structures as shown in Y4 to Y8, wherein an asterisk (x) position represents a position where the Y part is connected with the L part through covalent bonds; 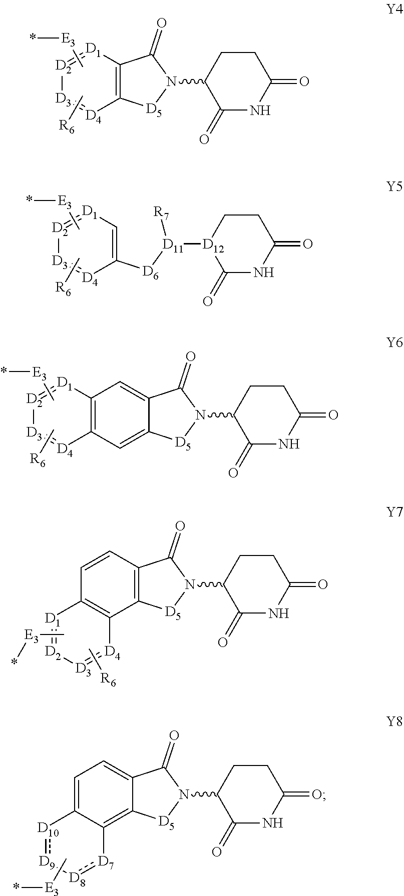
wherein,
D₁, D₂, D₃ and D₄ are selected from 0 to 2 N, CH, or CR₆ independently from each other;
preferably, the D₁, the D₂, the D₃ and the are independent of each other as CH or CR₆; or, when at least one of the D₁, the D₂, the D₃ or the D₄ is CH or CR₆, at least one of the remaining D₁, D₂, D₃ or D₄ is N;
the D₅ is selected from (CH₂)_{q}—, —CF₂, CO, —W₁ [symbol] W₂— or —W₁ [symbol] W₂ [symbol] W₃—, wherein, q is optionally selected from natural numbers 0 to 5; the W₁, W₂ or W₃ are selected from CR₇R₈, N, NH, CO or CF₂ independently from each other, wherein, W₁ and W₂, as well as W₂ and W₃ are independently connected by covalent single bonds or double bonds respectively; and the R₇ or the R₈ are selected from H or a C1-4 linear or branched alkyl independently from each other;
the D₆ is selected from CH_{N} or CO, or is a covalent bond;
the D₁₁ is selected from NR₇, or is a covalent bond;
the D₁₂ is selected from CH or N;
in the Y8, at least one of D₇, D₈, D₉ and D₁₀ is N or a covalent bond, while the remaining is selected from N, O, S, or CH₂ independently from each other;
the E₃ and the R₆ are connected with D₁, D₂, D₃ or D₄ through covalent bonds independently from each other, and the D₁, the D₂, the D₃ or the D₄ at a connecting site is CH or CR₆C;
preferably, the E₃ is selected from NH, CH₂, CF₂, O, —CH═CH—, —C≡C—, —Cy₄ or Cy₄-Cy₅;
preferably, the R₆ is selected from H, F, Cl, OR₇ or NHR₇;
the Cy₄ is selected from a 4-7-membered carbon heteromonocyclic ring, a 5-10-membered carbon heterofused ring, a 4-7-membered monocyclic alkyl, a 5-10-membered cycloalkyl, a 7-10-membered bridged cycloalkyl, or a 5-8-membered aromatic ring group;
preferably, the aromatic ring group, the monocyclic alkyl, the carbon heteromonocyclic ring and the carbon heterofused ring are optionally substituted by 0 to 3 substituents selected from H, F, Cl, OH, COOH, CN, NH₂, a carbonyl, a C1-4 linear or branched alkyl, a halogen-substituted C1-4 linear or branched alkyl, a hydroxyl-substituted C1-4 linear or branched alkyl or a C1-4 alkoxy;
preferably, the heteromonocyclic ring and the heterofused ring contain 0 to 4 heteroatoms selected from O, S and N; and the aromatic ring group contains 0 to 3 heteroatoms selected from N, S and O;
preferably, the Cy₄ is selected from any substituted piperidine, piperazine, or azetidine;
the Cy₅ is selected from a 4-7-membered carbon heteromonocyclic ring, a 5-10-membered carbon heterofused ring, a 4-7-membered monocyclic alkyl, a 5-10-membered cycloalkyl, a 7-10-membered bridged cycloalkyl, or a 5-8-membered aromatic ring group;
preferably, the aromatic ring group, the monocyclic alkyl, the carbon heteromonocyclic ring and the carbon heterofused ring are optionally substituted by 0 to 3 substituents selected from H, F, Cl, OH, COOH, CN, NH₂, a carbonyl, a C1-4 linear or branched alkyl, a halogen-substituted C1-4 linear or branched alkyl, a hydroxyl-substituted C1-4 linear or branched alkyl or a C1-4 alkoxy;
preferably, the heteromonocyclic ring and the heterofused ring contain 0 to 4 heteroatoms selected from O, S and N; and the aromatic ring group contains 0 to 3 heteroatoms selected from N, S and O;
preferably, the Cy₅ is selected from any substituted azetidine, piperidine, or piperazine.

6. The bifunctional compound of claim 1, wherein the L part is a covalent bond, and directly connects the X part with the Y part;
or,
the L part is selected from —O—, —C═O, —OCO—, —CH₂(CH₂)ₙCO—, —CH₂(CH₂)ₙ—, —CONHCH₂(CH₂)ₙCO—, —COCH₂(CH₂)ₙCO—, —O(CH₂)ₙ—, —O(CH₂)ₙCO—, —O(CH₂)ₙNH—, —NH(CH₂)ₙCO—, —CH₂CH₂N(R₃)CO—, —CH₂OCH₂CO—, or —CH₂Ar₁CO—, wherein the Ar₁ is selected from a benzene ring, a thiophene ring, or a 1H-pyrrole; and n is optionally natural numbers 0 to 14;
or,
the L part is selected from structures as shown below, wherein a zigzag line represents a position where the L part is connected with the X part through covalent bonds, and an asterisk (x) position represents a position where the L part is connected with the Y part through covalent bonds; 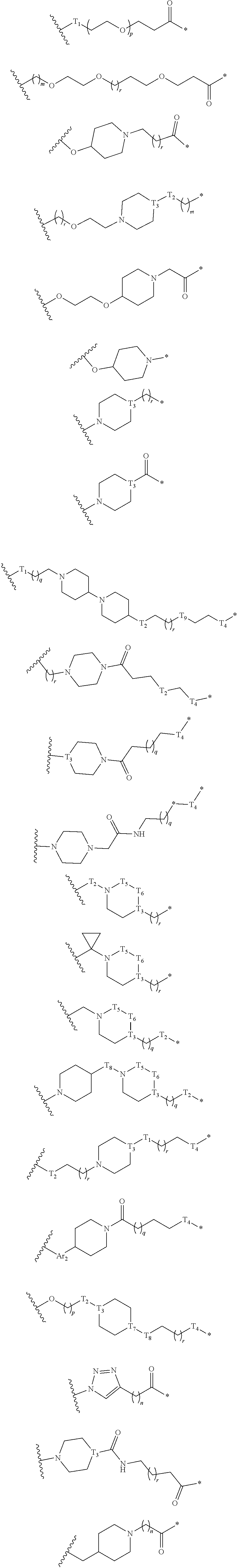 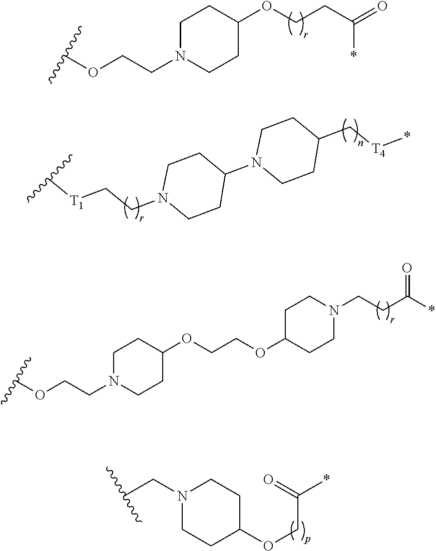 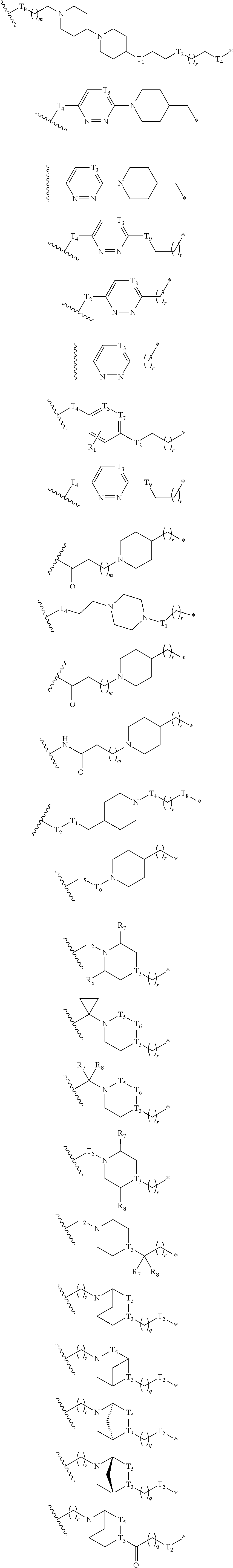 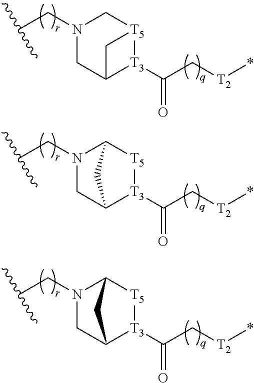 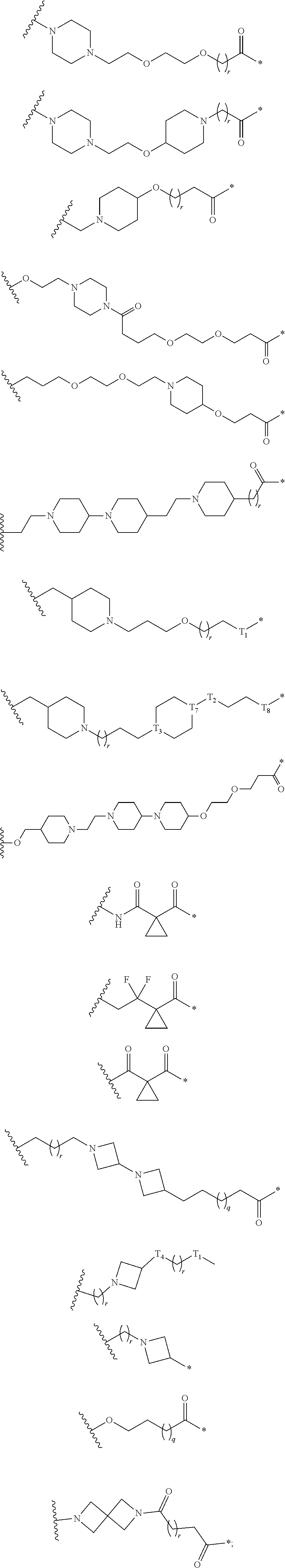
wherein,
the T₁ is selected from O, CH₂ or CO;
the T₂ is selected from O, CH₂, CF₂, NH or CO;
the T₃ and T₇ are selected from N or CH independently from each other;
the T₄ is selected from CH₂ or CO;
the T₅ and T₆ are selected from CH₂, CF₂ or CO independently from each other;
the T₈ is selected from O, CH₂, CF₂, NH or CO;
the T₉ is selected from O, NH or CH₂;
the Ar₂ is selected from a phenyl, an imidazole group or a pyrazolyl;
the R₈ is selected from H or a C1-4 linear or branched alkyl; and
m is optionally selected from natural numbers 0 to 2, n is optionally selected from natural numbers 0 to 12, p is optionally selected from natural numbers 1 to 6, q is optionally selected from natural numbers 0 to 5, and r is optionally selected from natural numbers 0 to 3.

7. The bifunctional compound of claim 1, wherein the compound is selected from any one of the following structures:

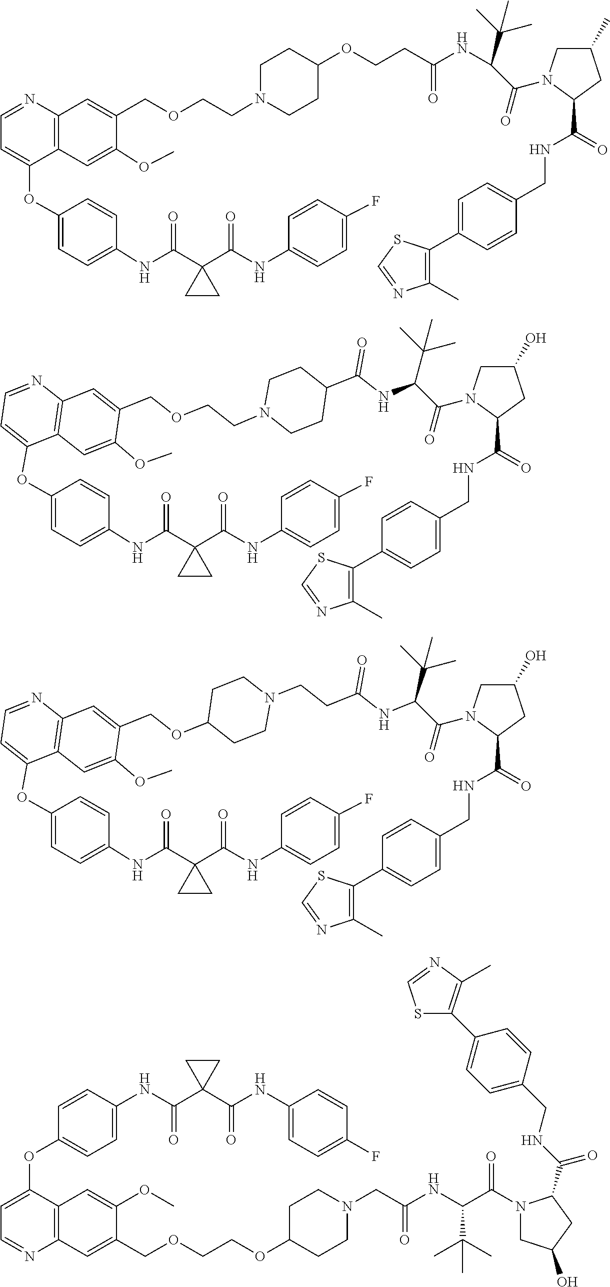

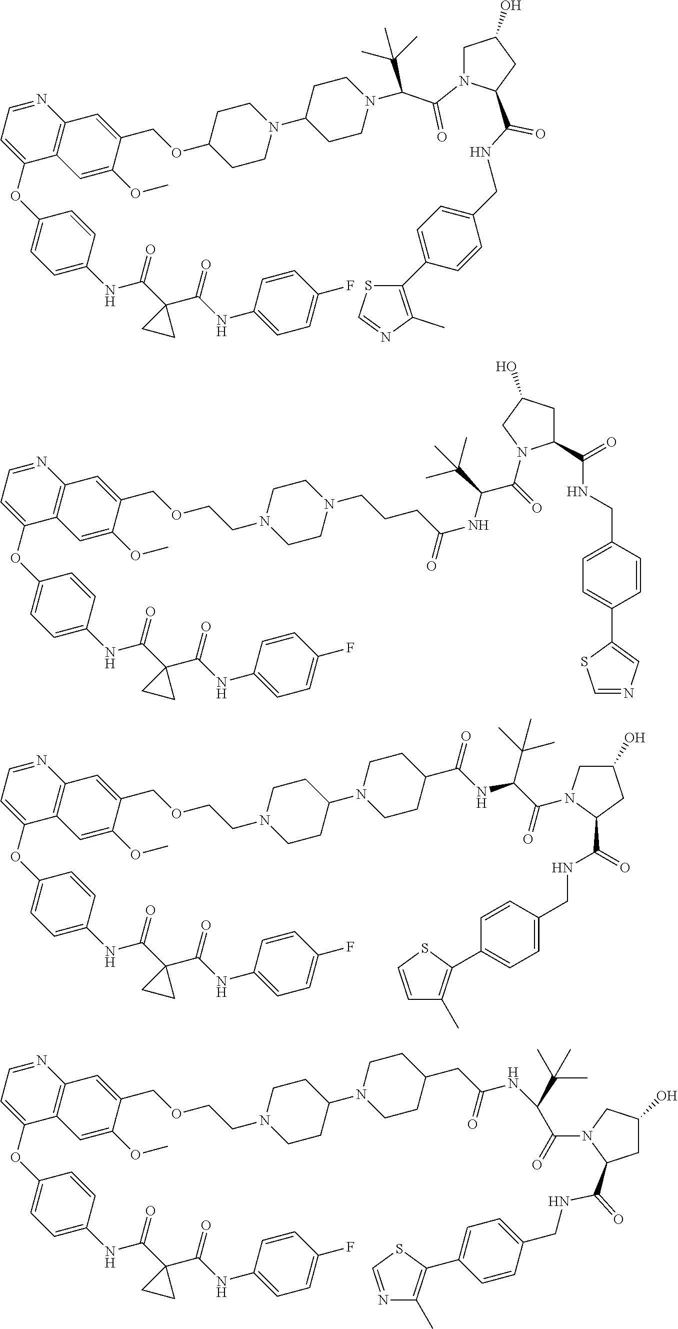

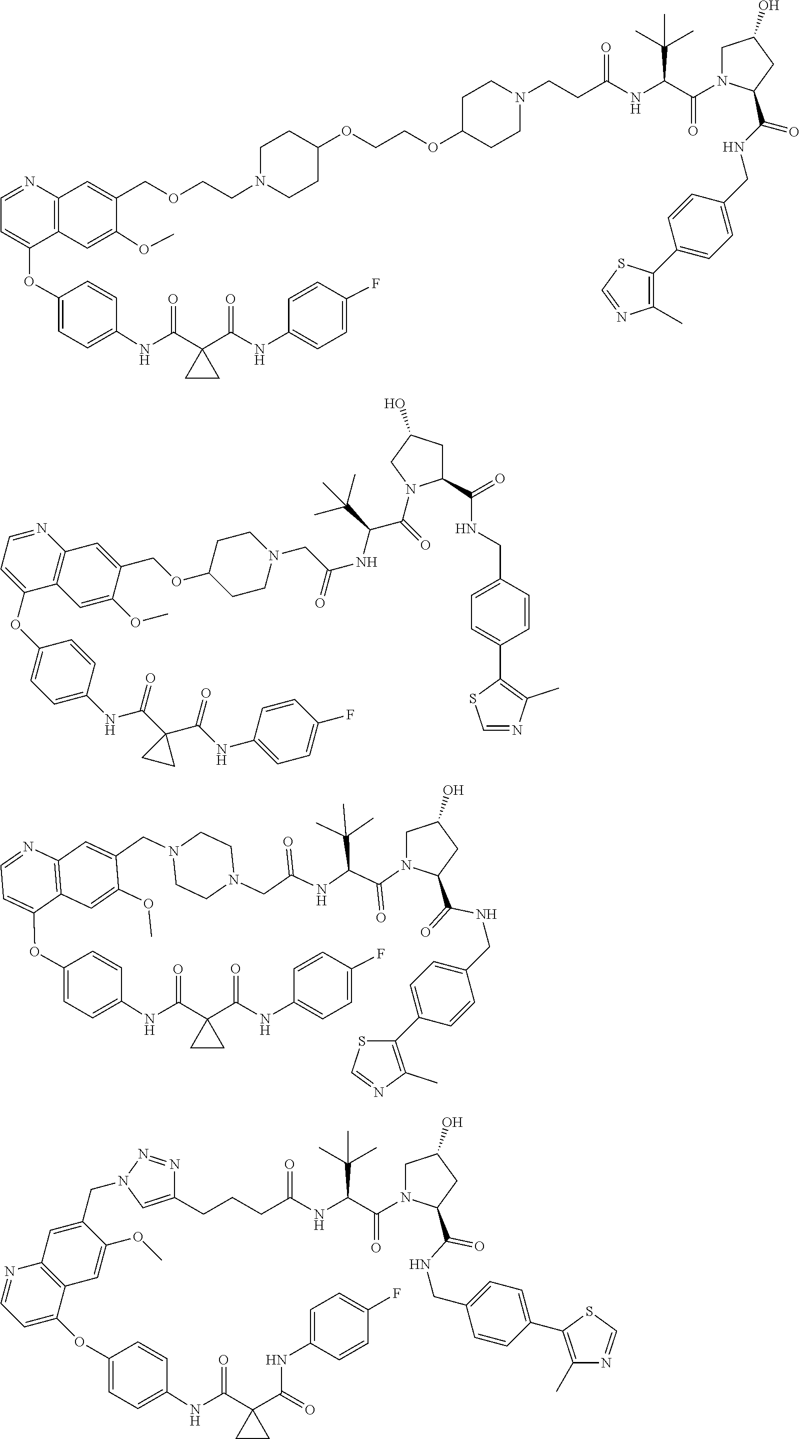

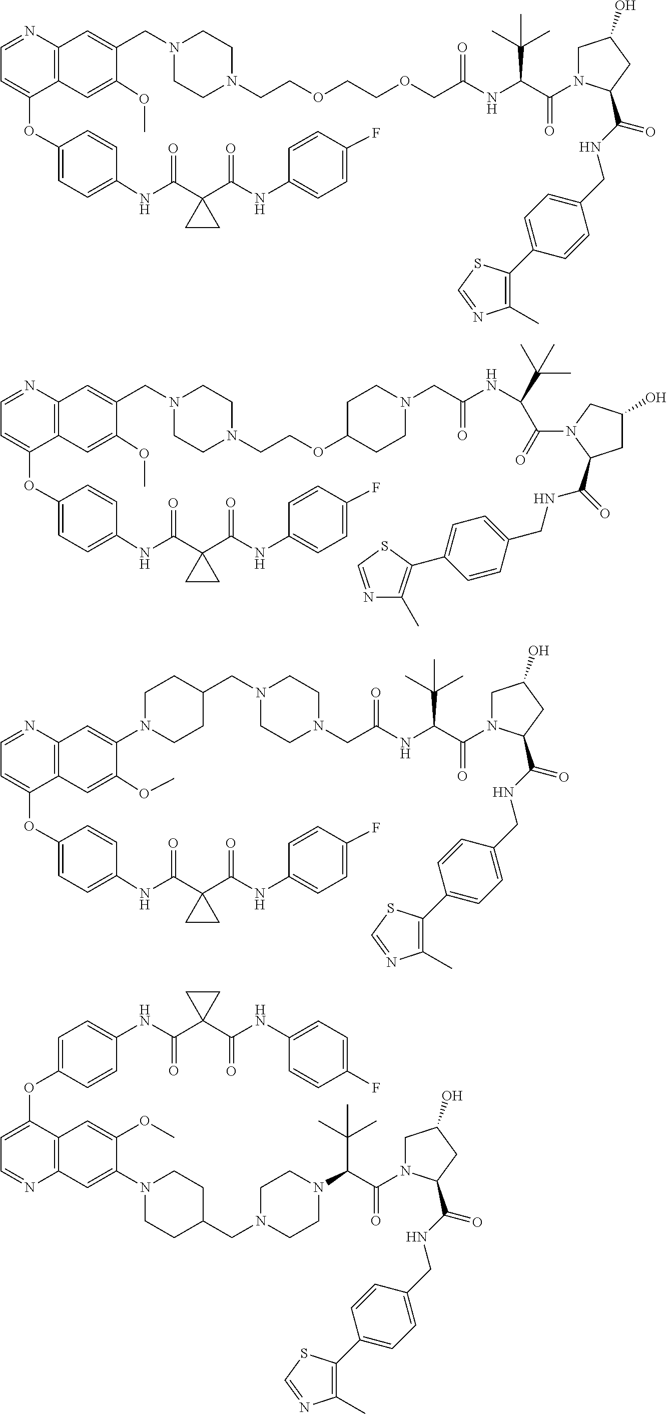

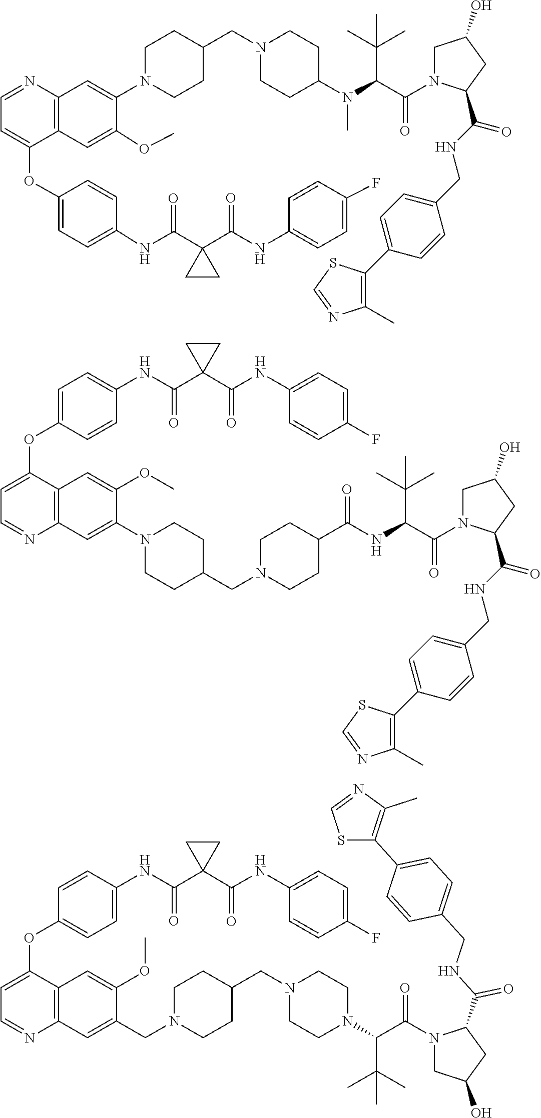

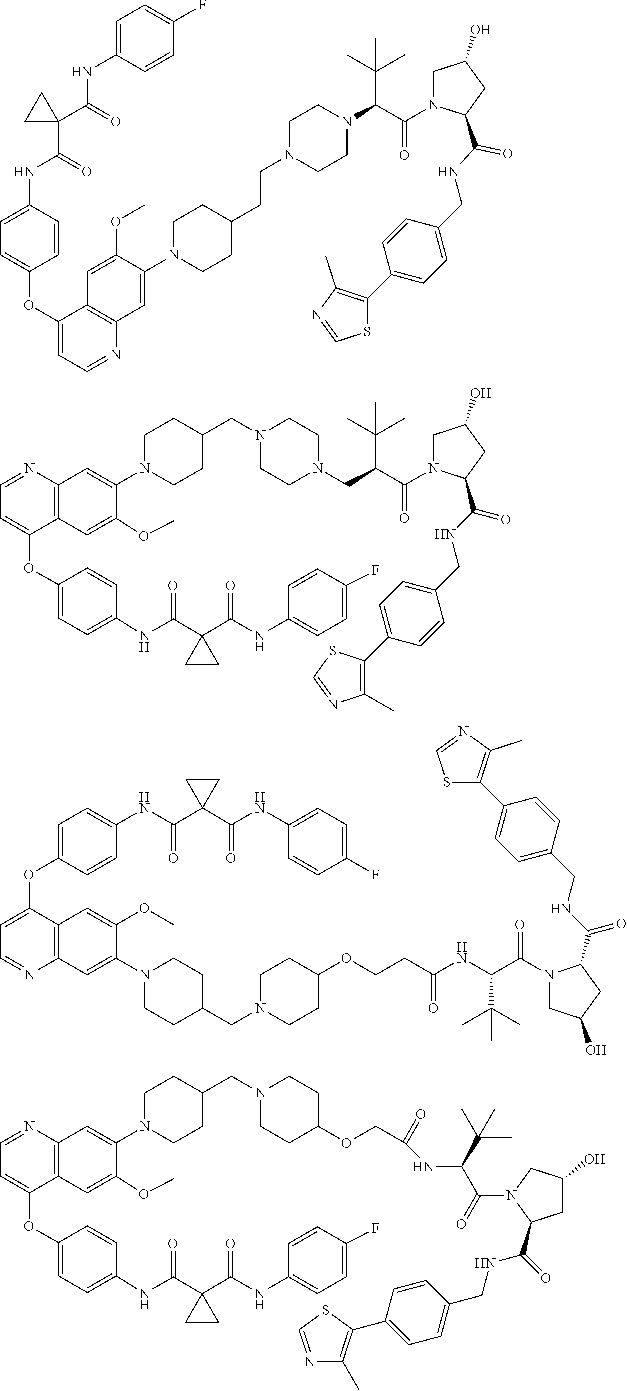

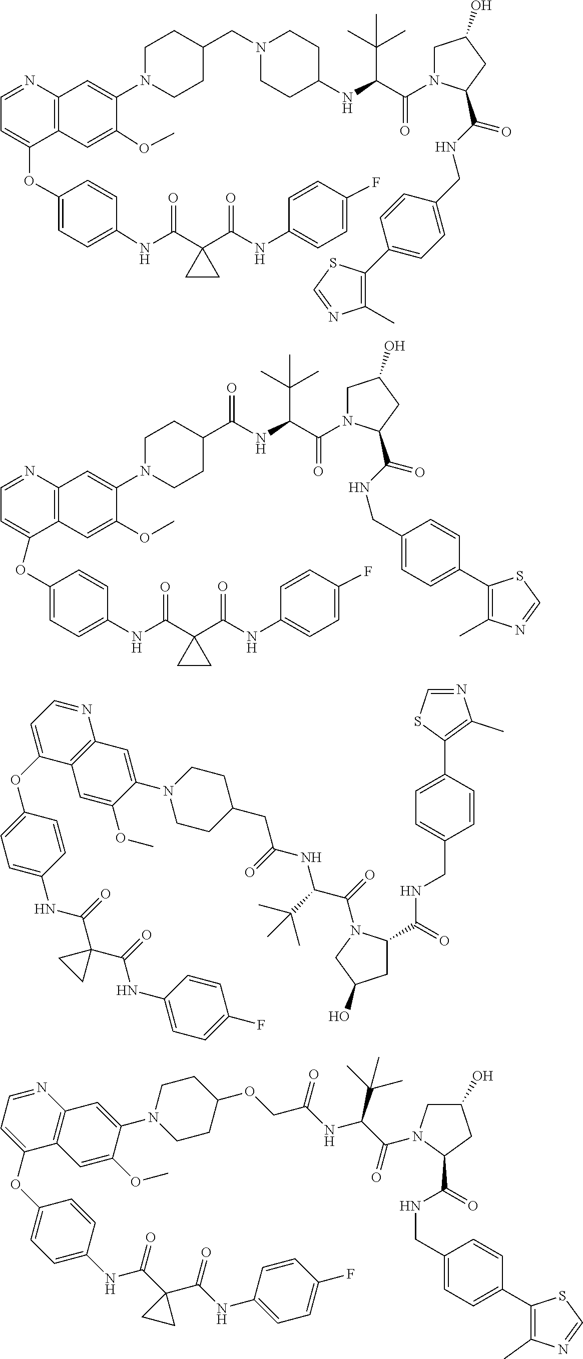

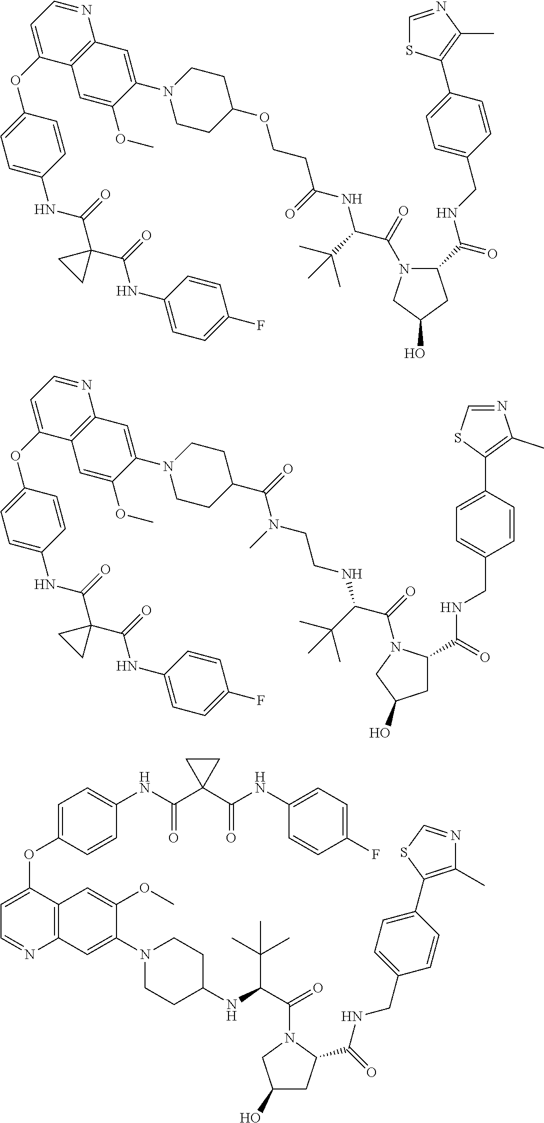

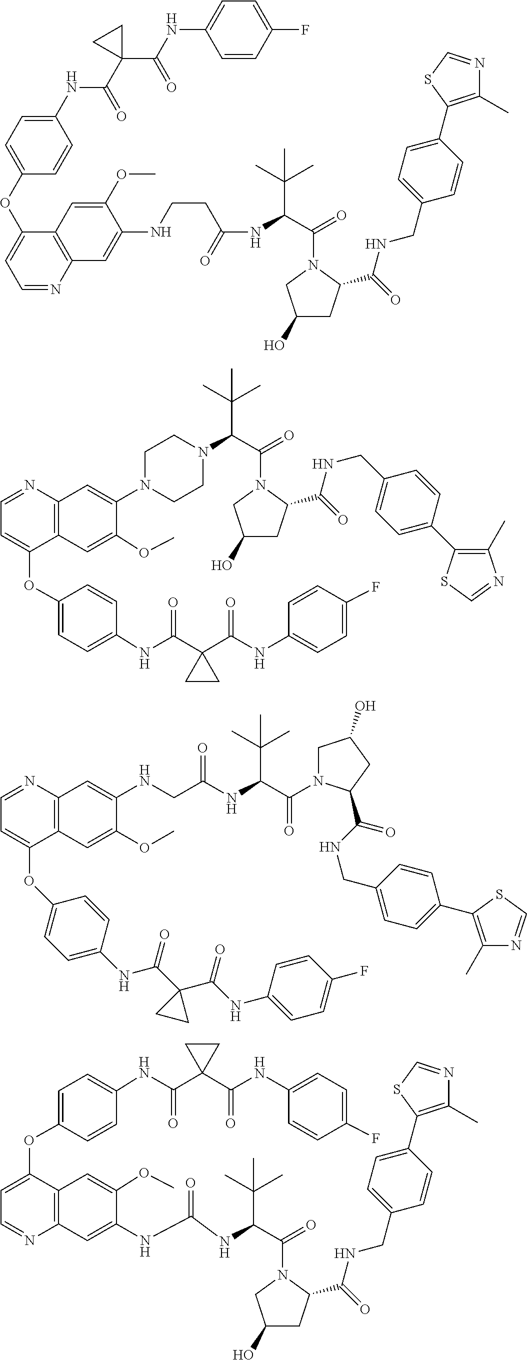

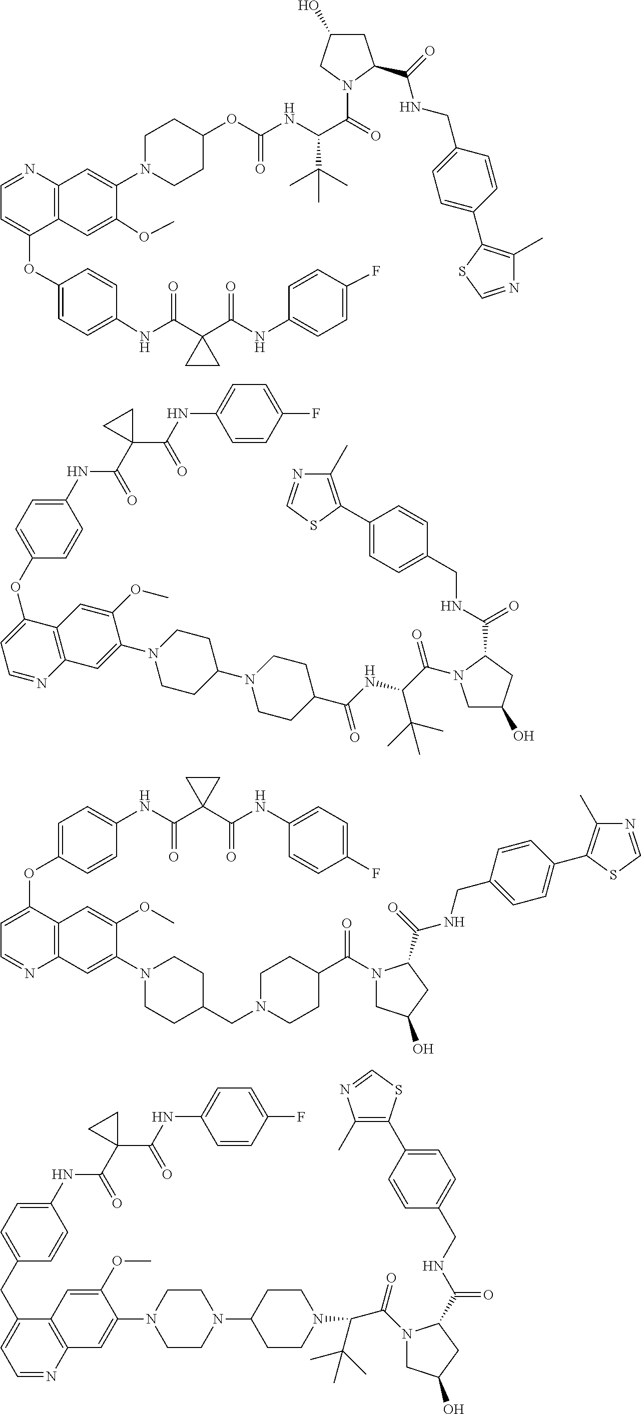

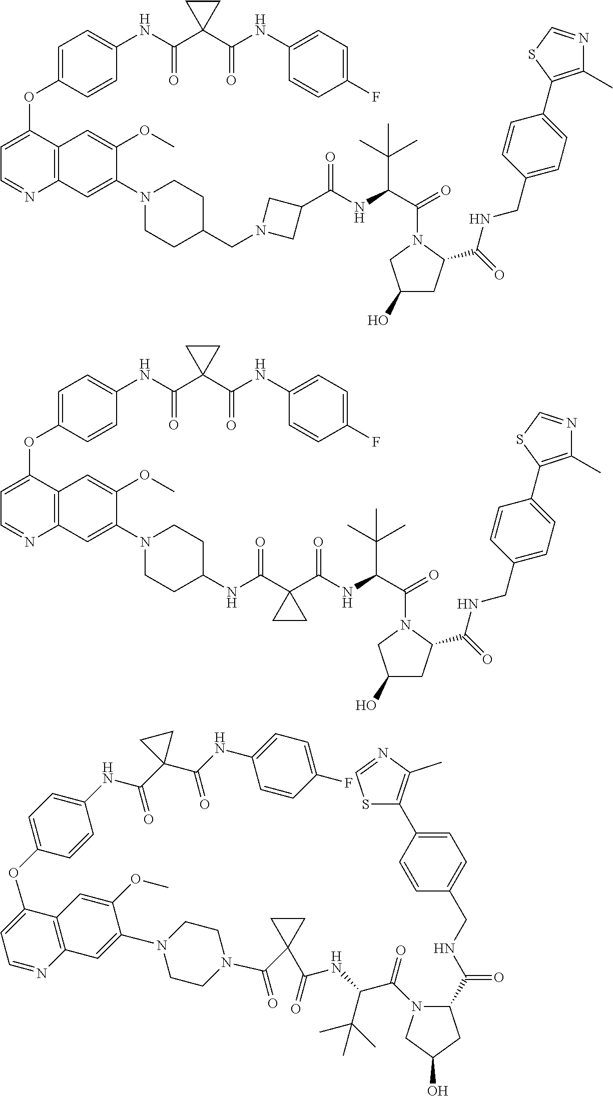 [SMILES: COc1cc2c(Oc3ccc(NC(=O)C4(C(=O)Nc5ccc(F)cc5)CC4)cc3)ccnc2cc1N1CCC(CN2CC(C(=O)N[C@H](C(=O)N3C[C@H](O)C[C@H]3C(=O)NCc3ccc(-c4scnc4C)cc3)C(C)(C)C)C2)CC1 ; COc1cc2c(Oc3ccc(NC(=O)C4(C(=O)Nc5ccc(F)cc5)CC4)cc3)ccnc2cc1N1CCC(NC(=O)C2(C(=O)N[C@H](C(=O)N3C[C@H](O)C[C@H]3C(=O)NCc3ccc(-c4scnc4C)cc3)C(C)(C)C)CC2)CC1 ; COc1cc2c(Oc3ccc(NC(=O)C4(C(=O)Nc5ccc(F)cc5)CC4)cc3)ccnc2cc1N1CCN(C(=O)C2(C(=O)N[C@H](C(=O)N3C[C@H](O)C[C@H]3C(=O)NCc3ccc(-c4scnc4C)cc3)C(C)(C)C)CC2)CC1]

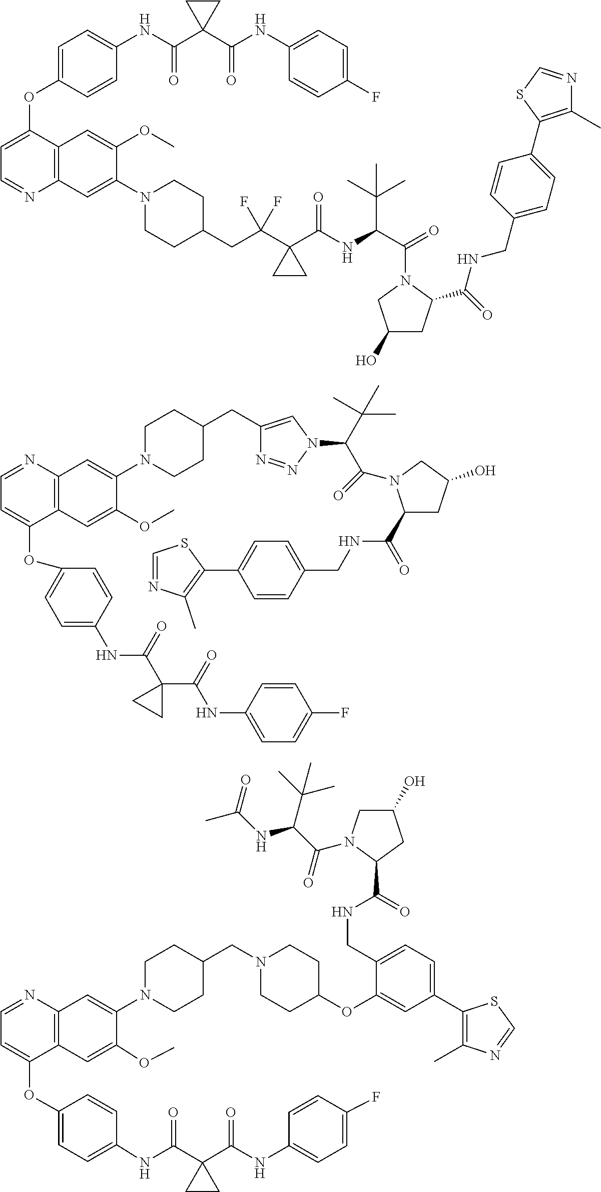

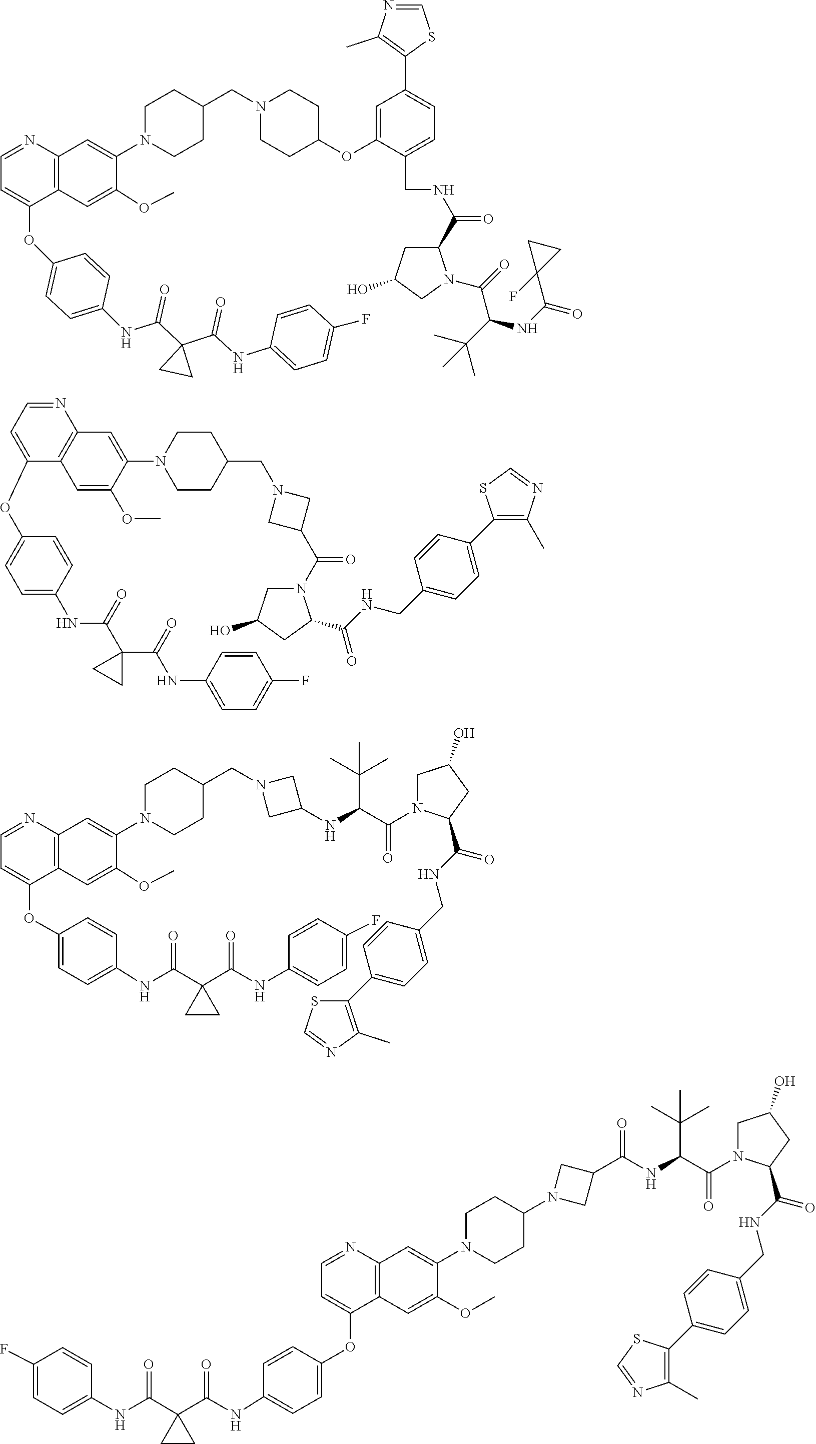

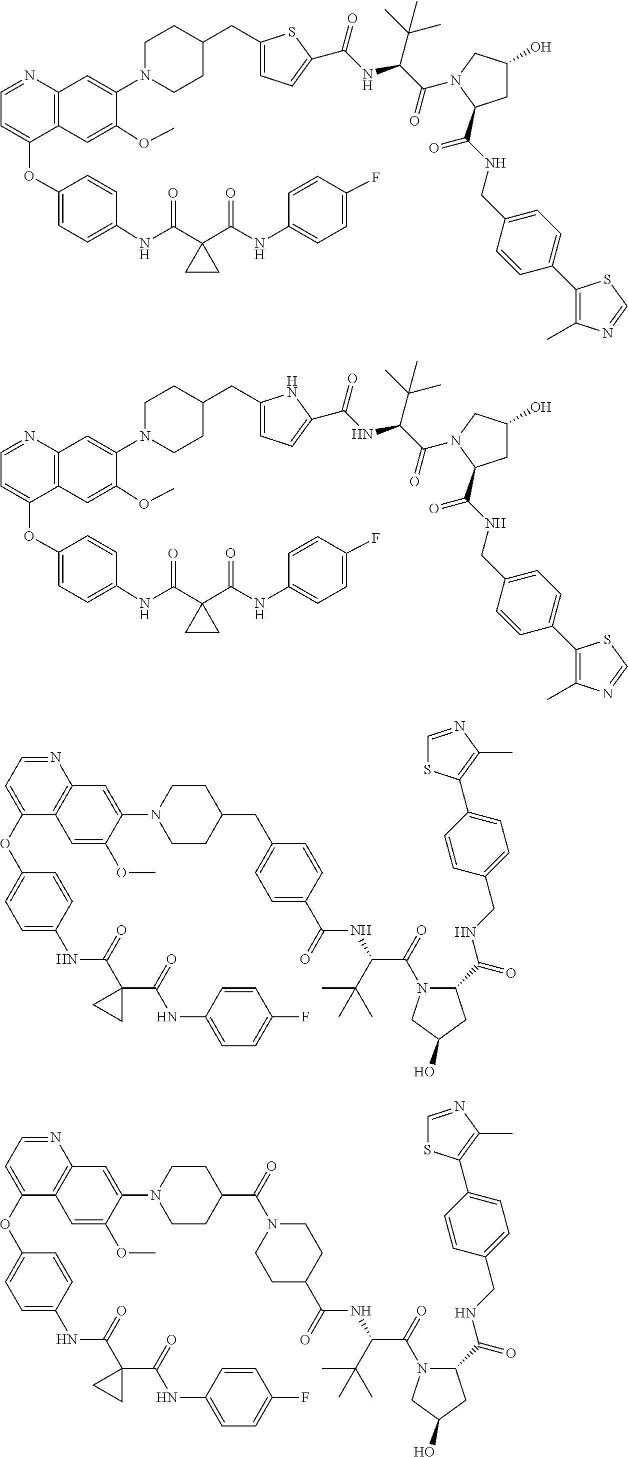

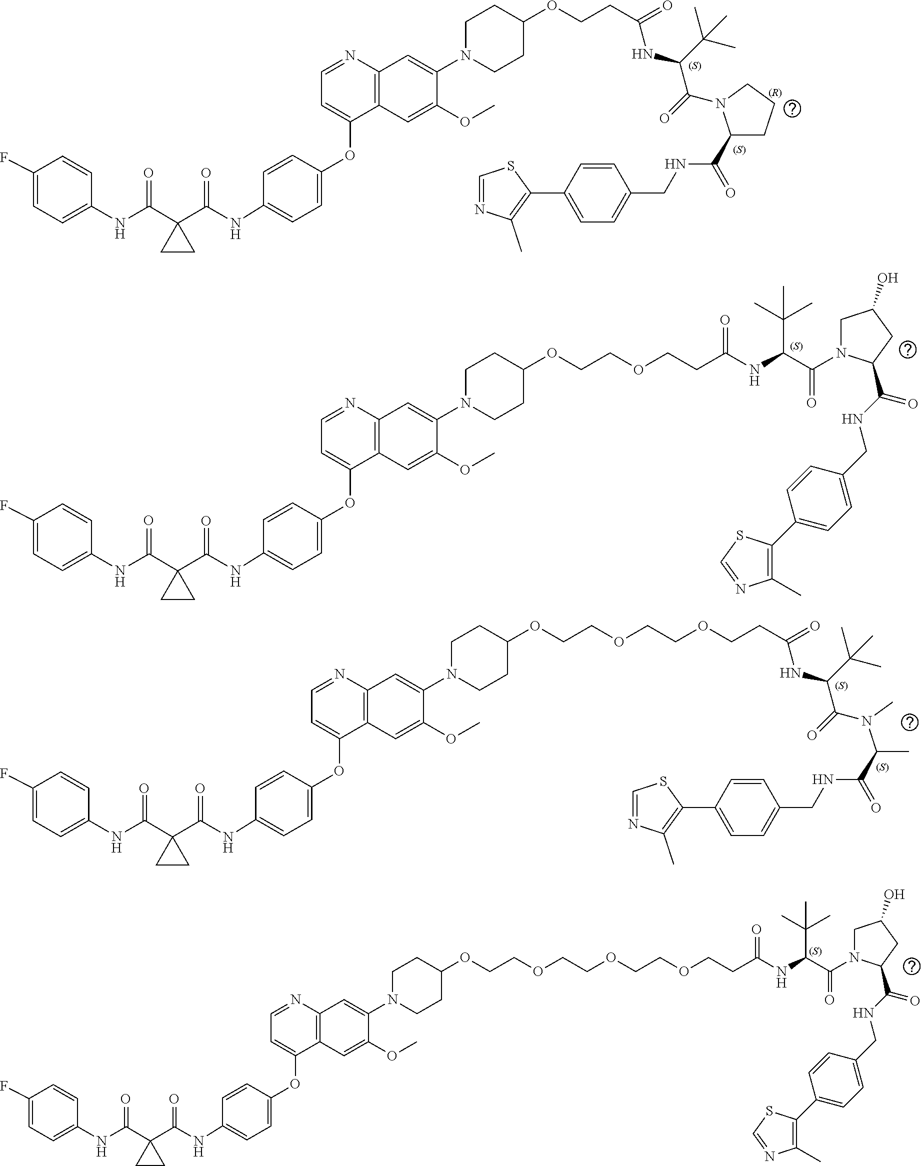

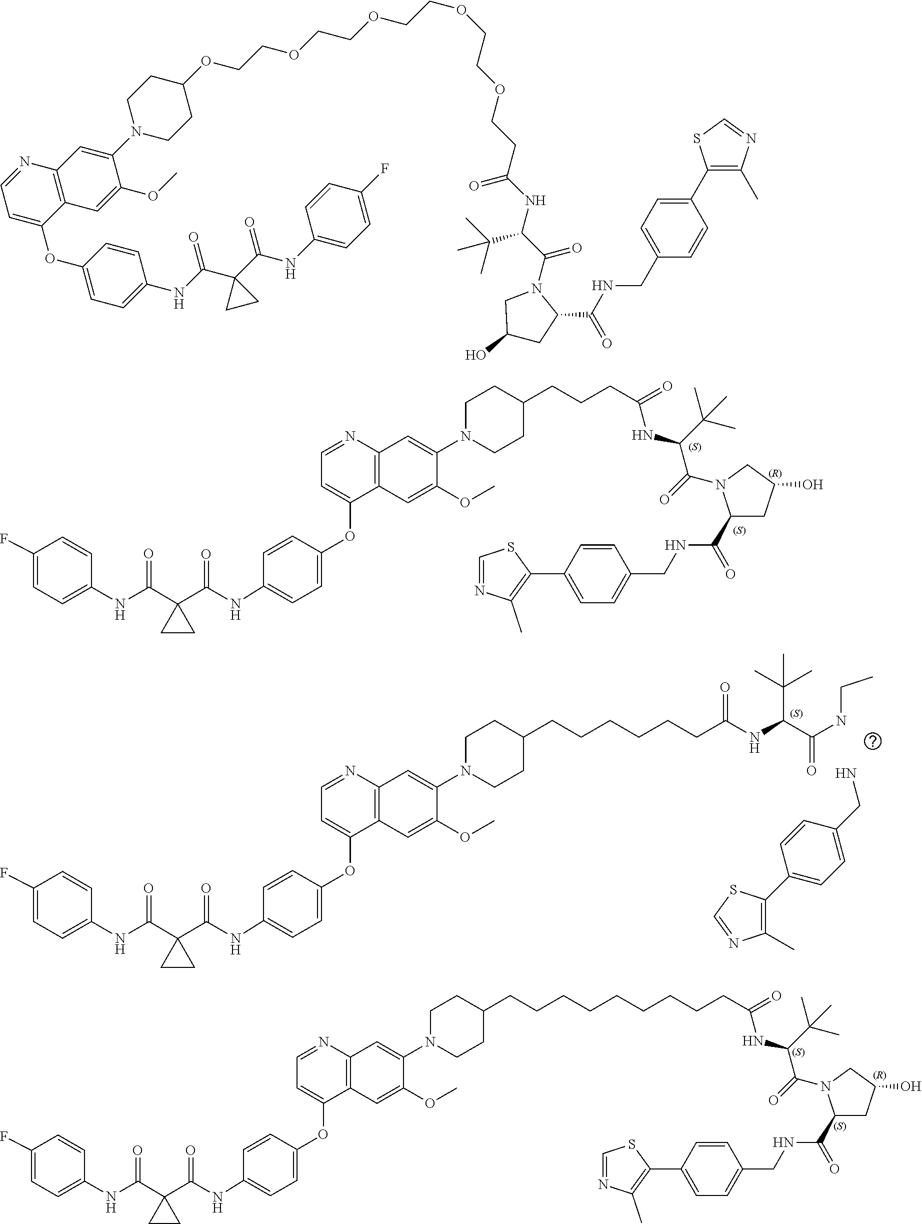

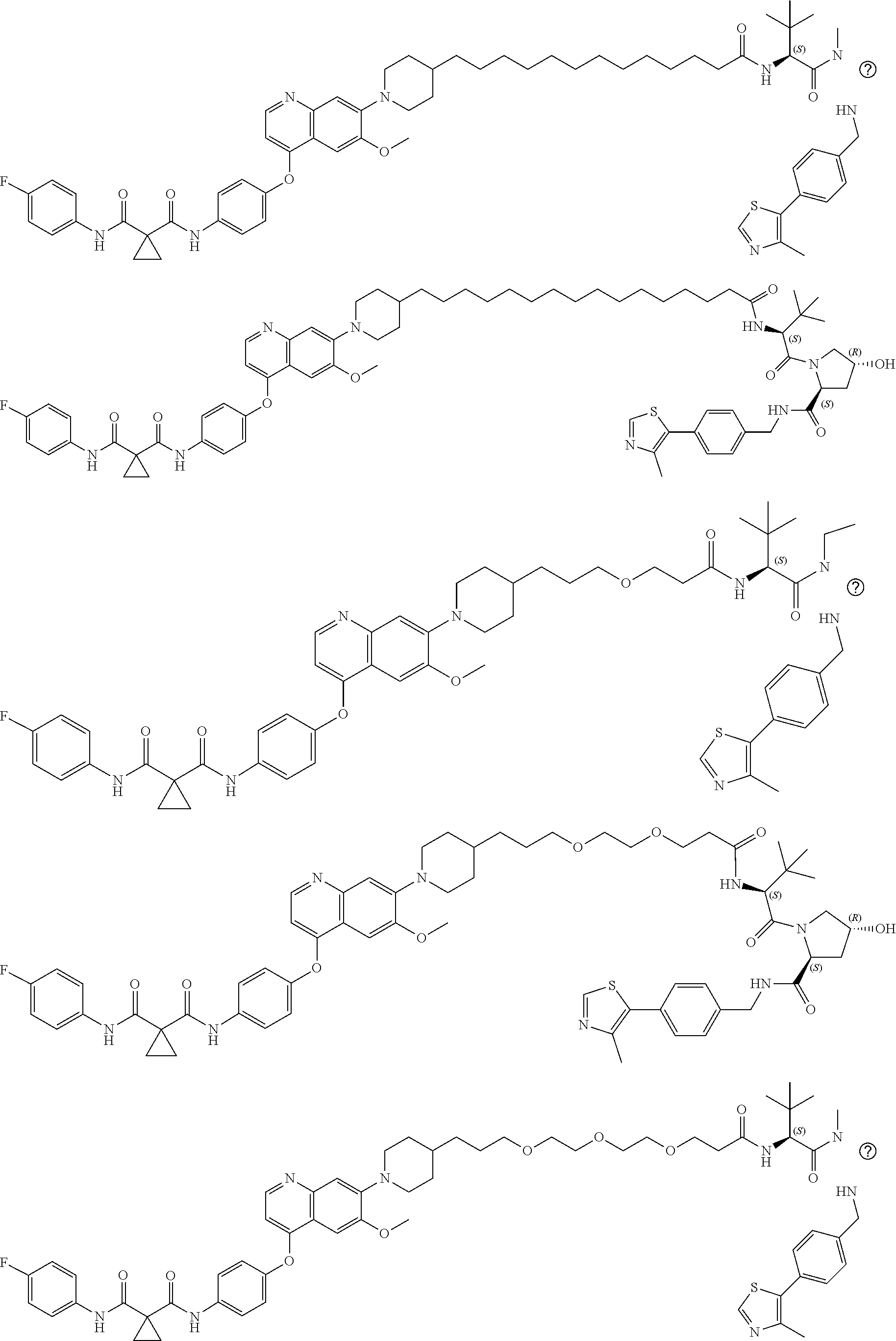

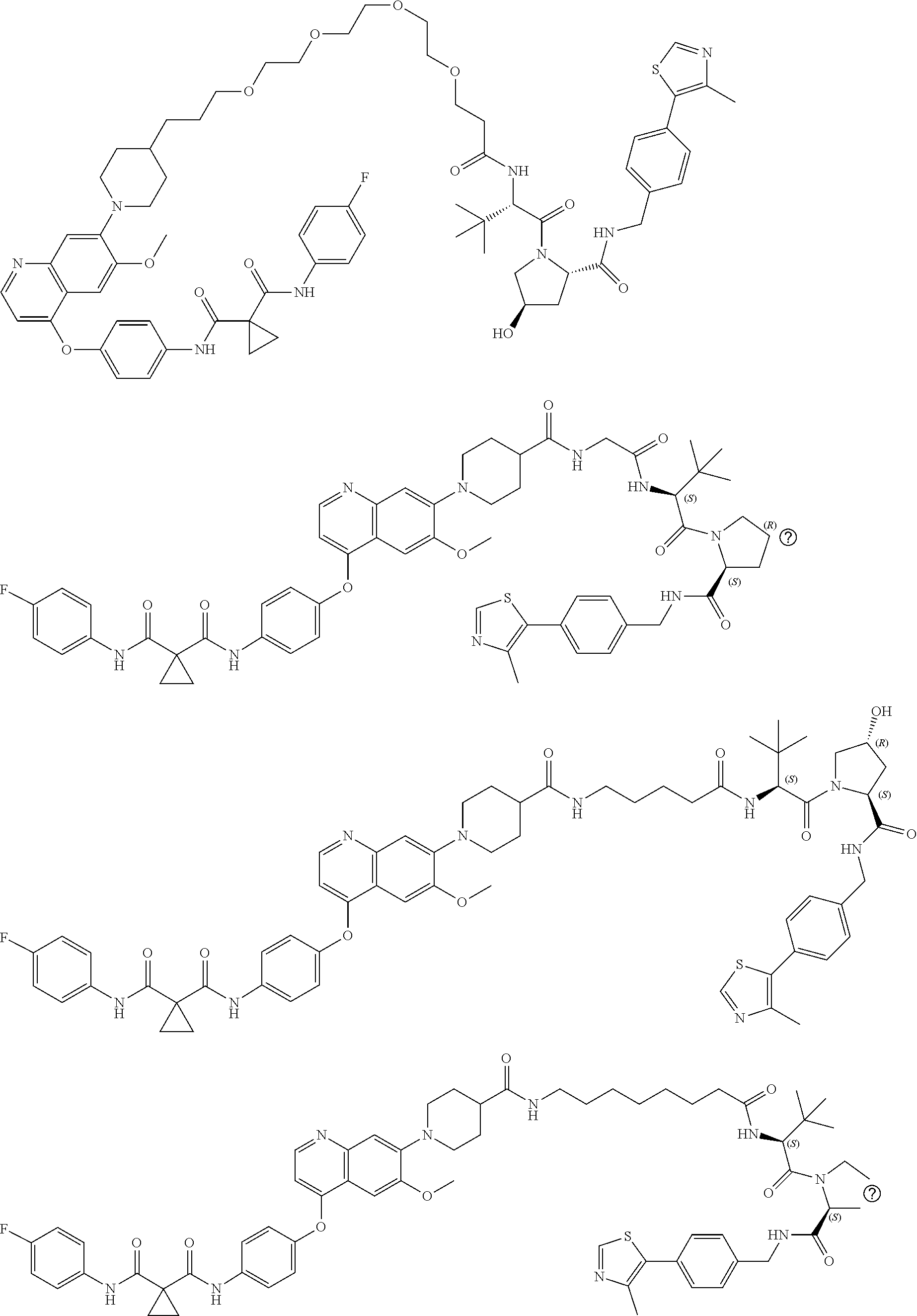

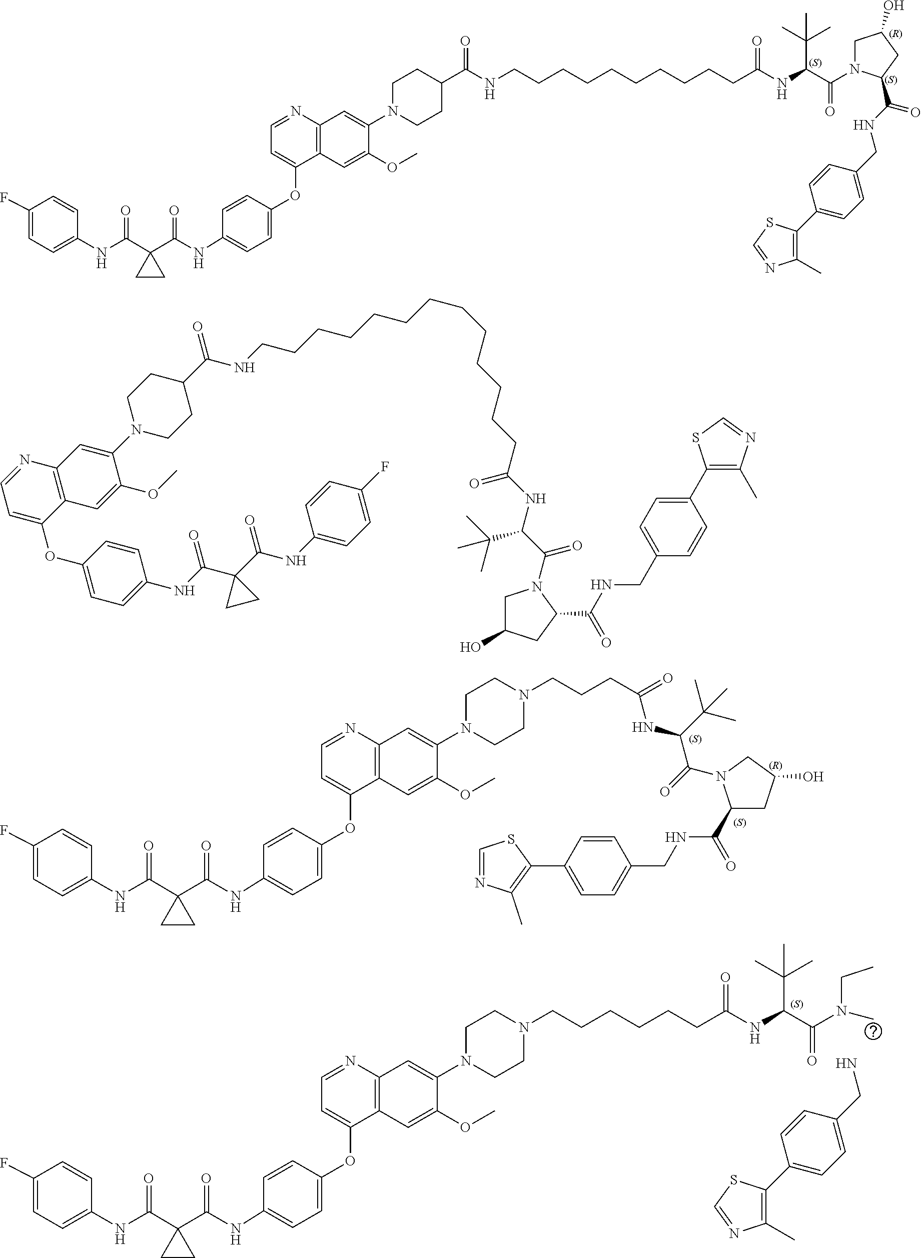

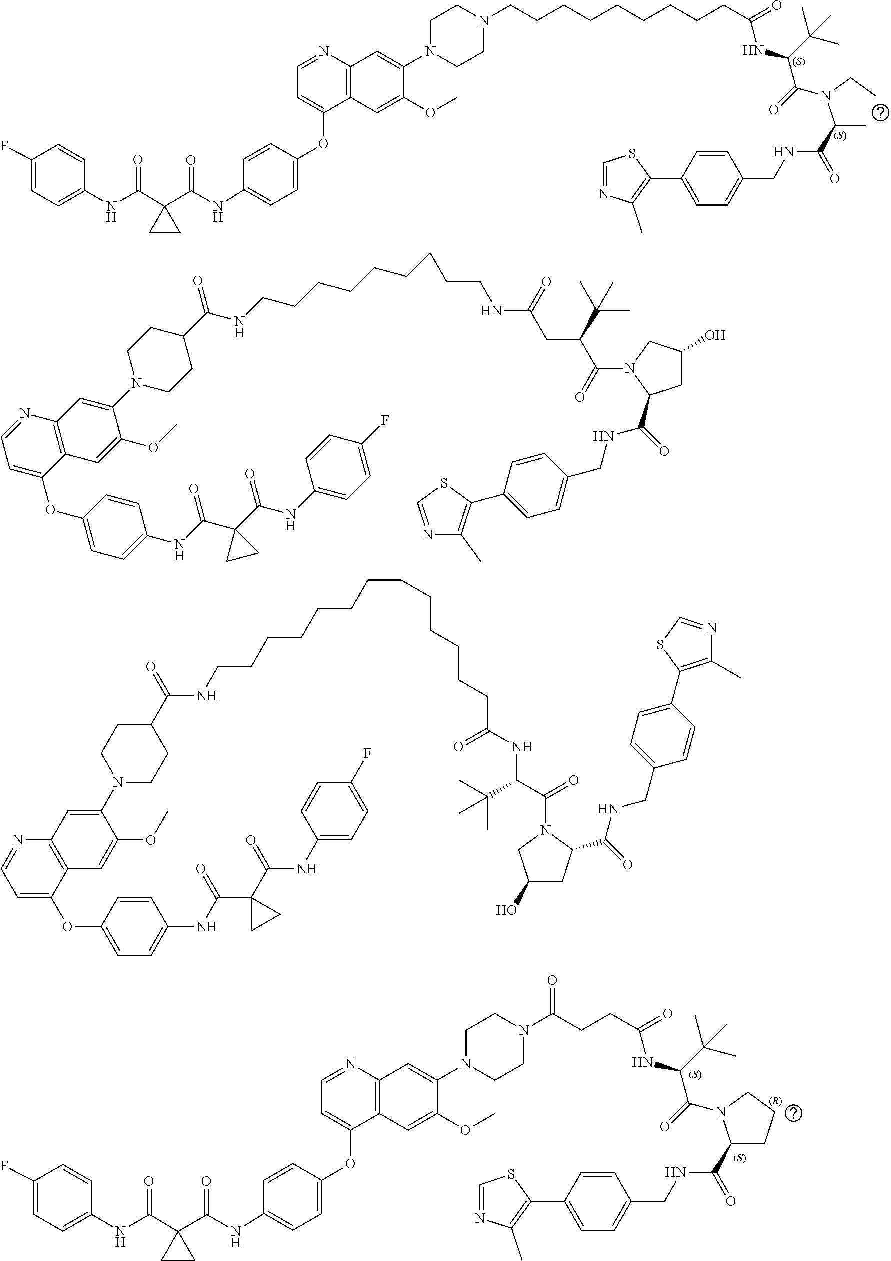

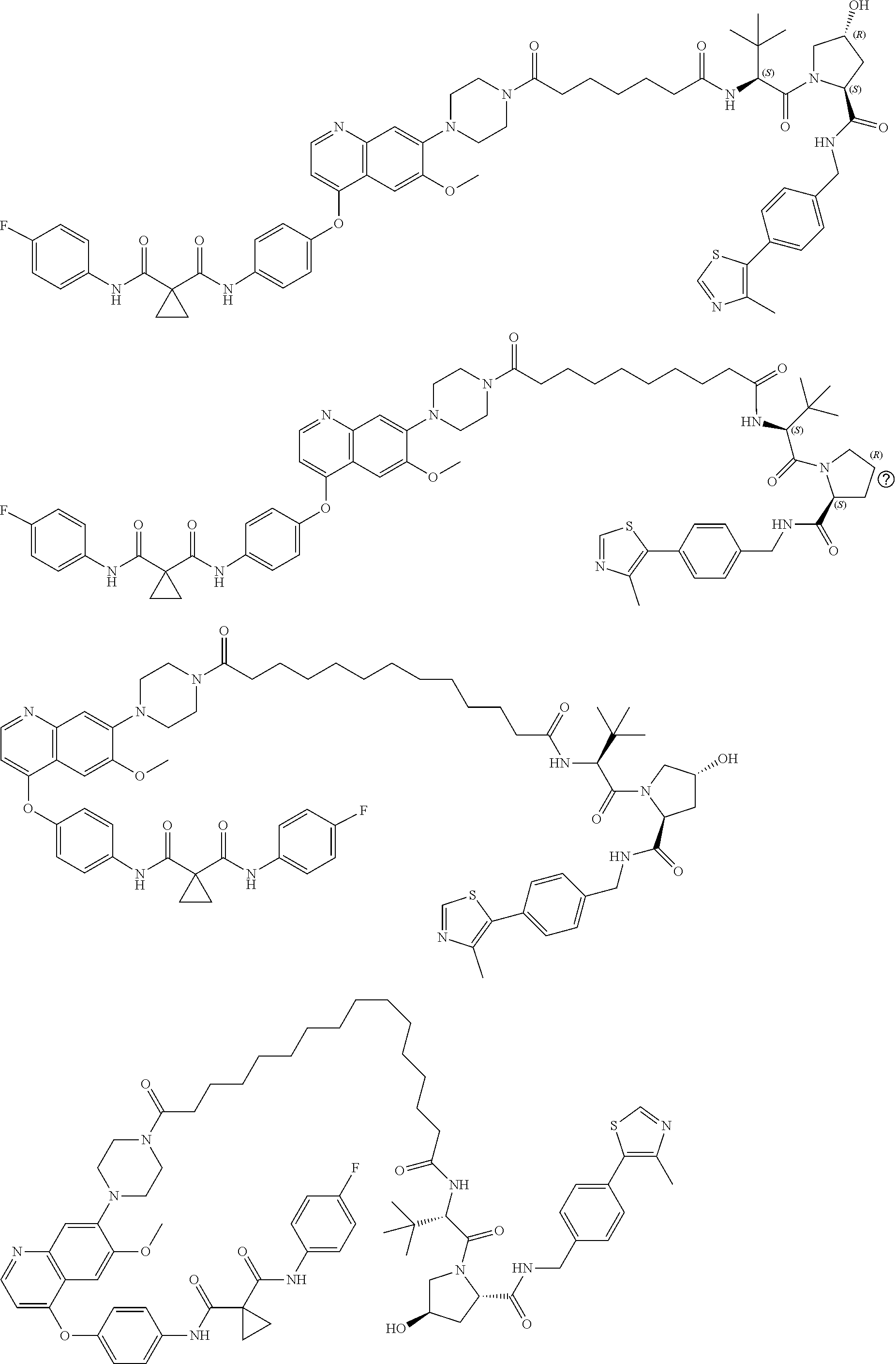

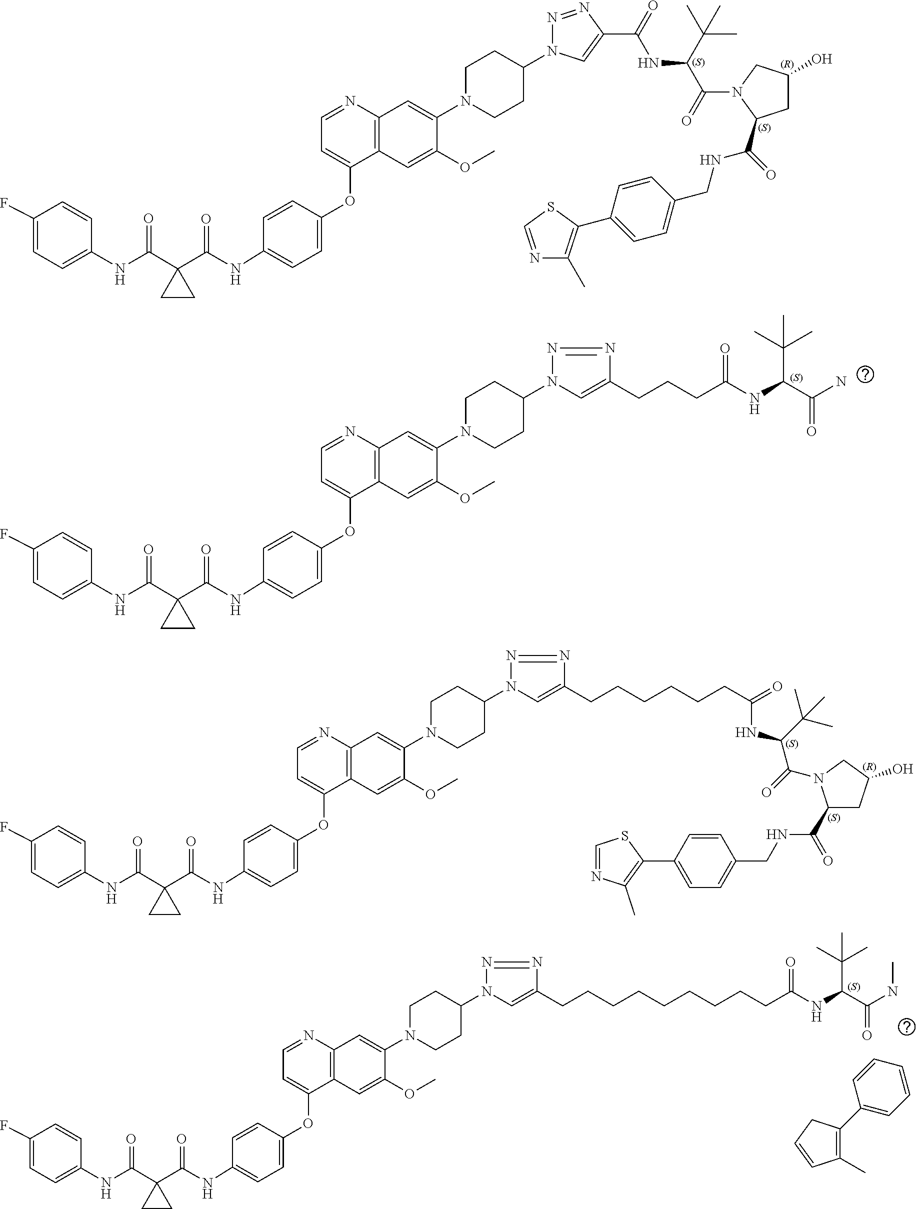

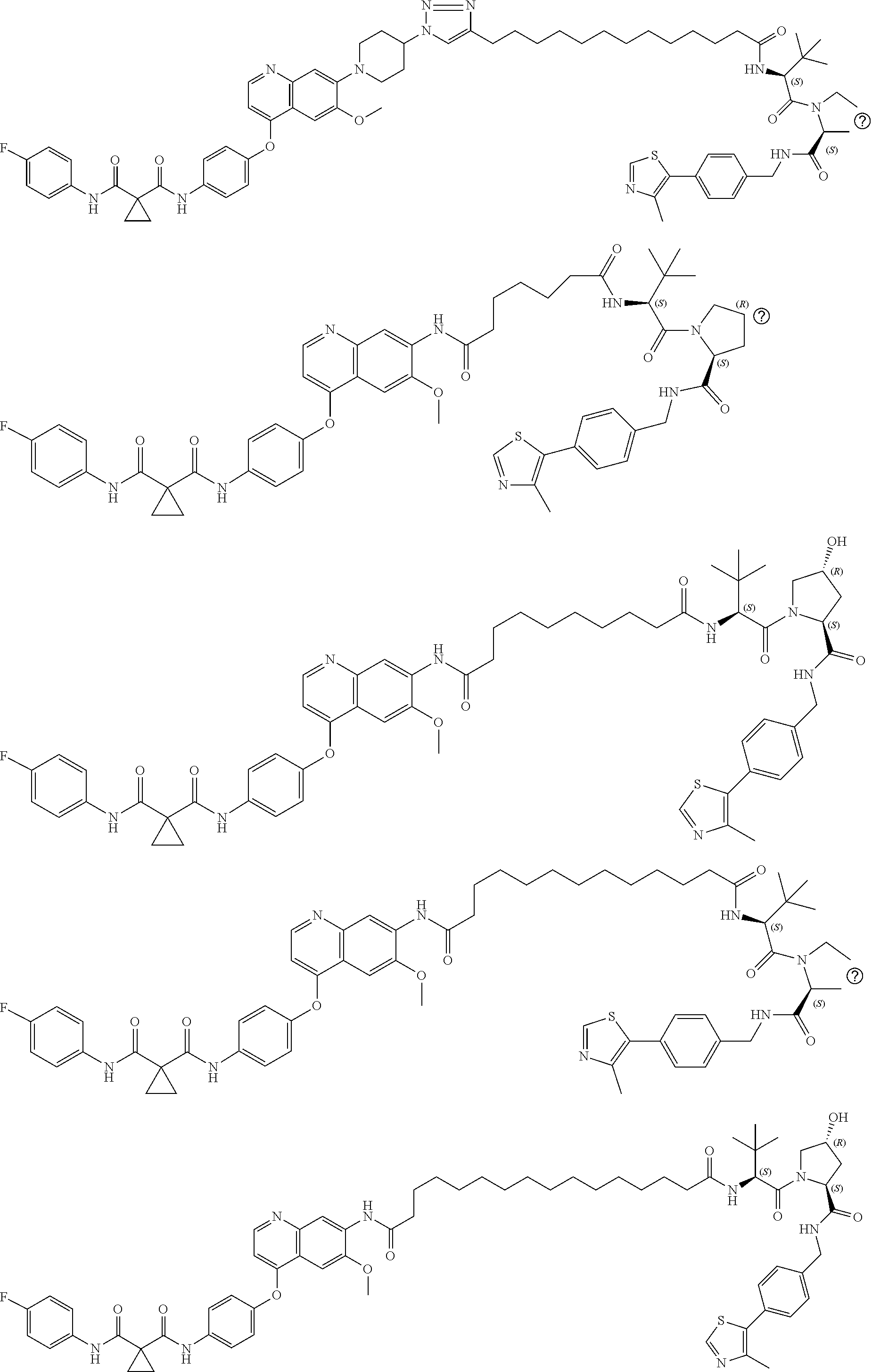

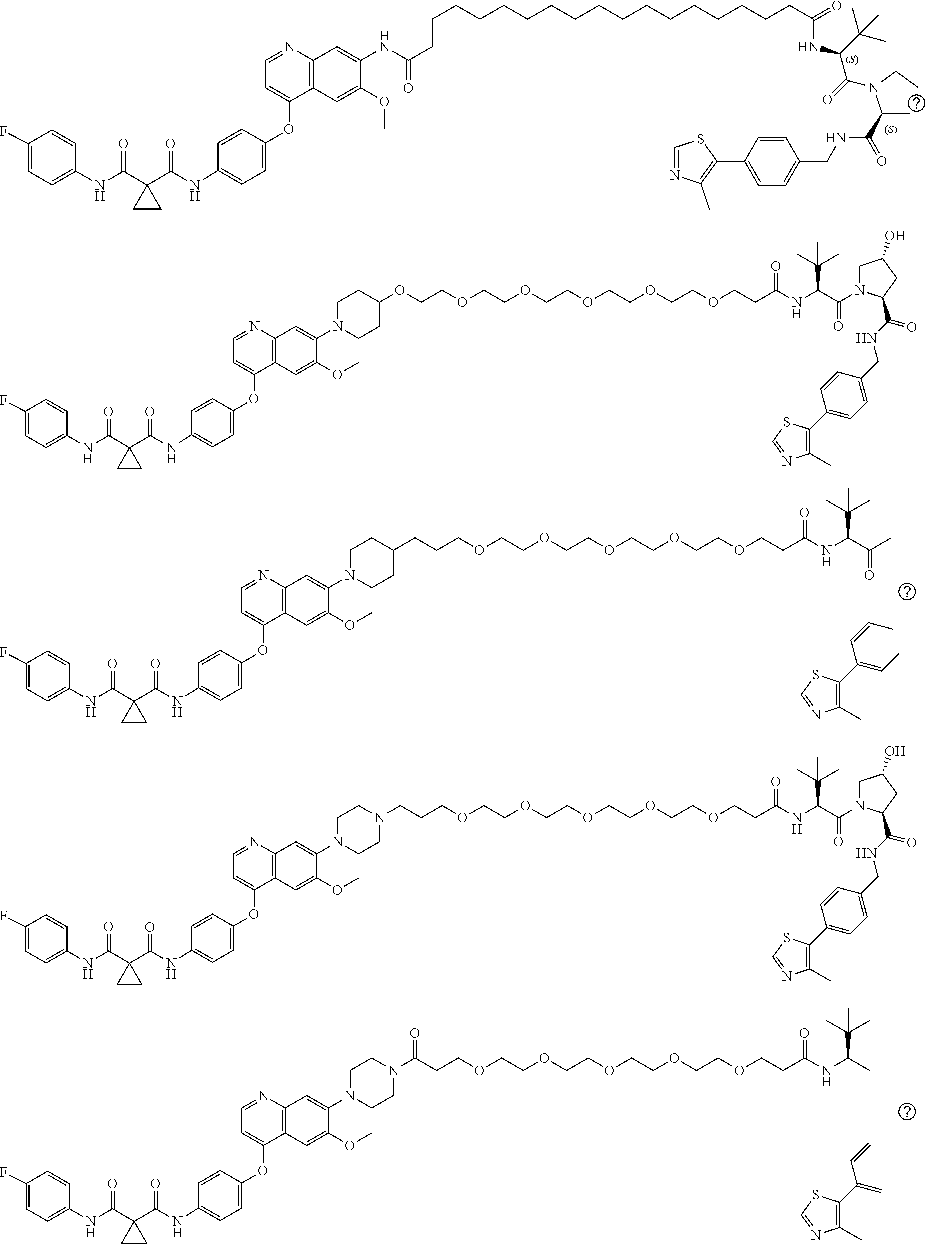

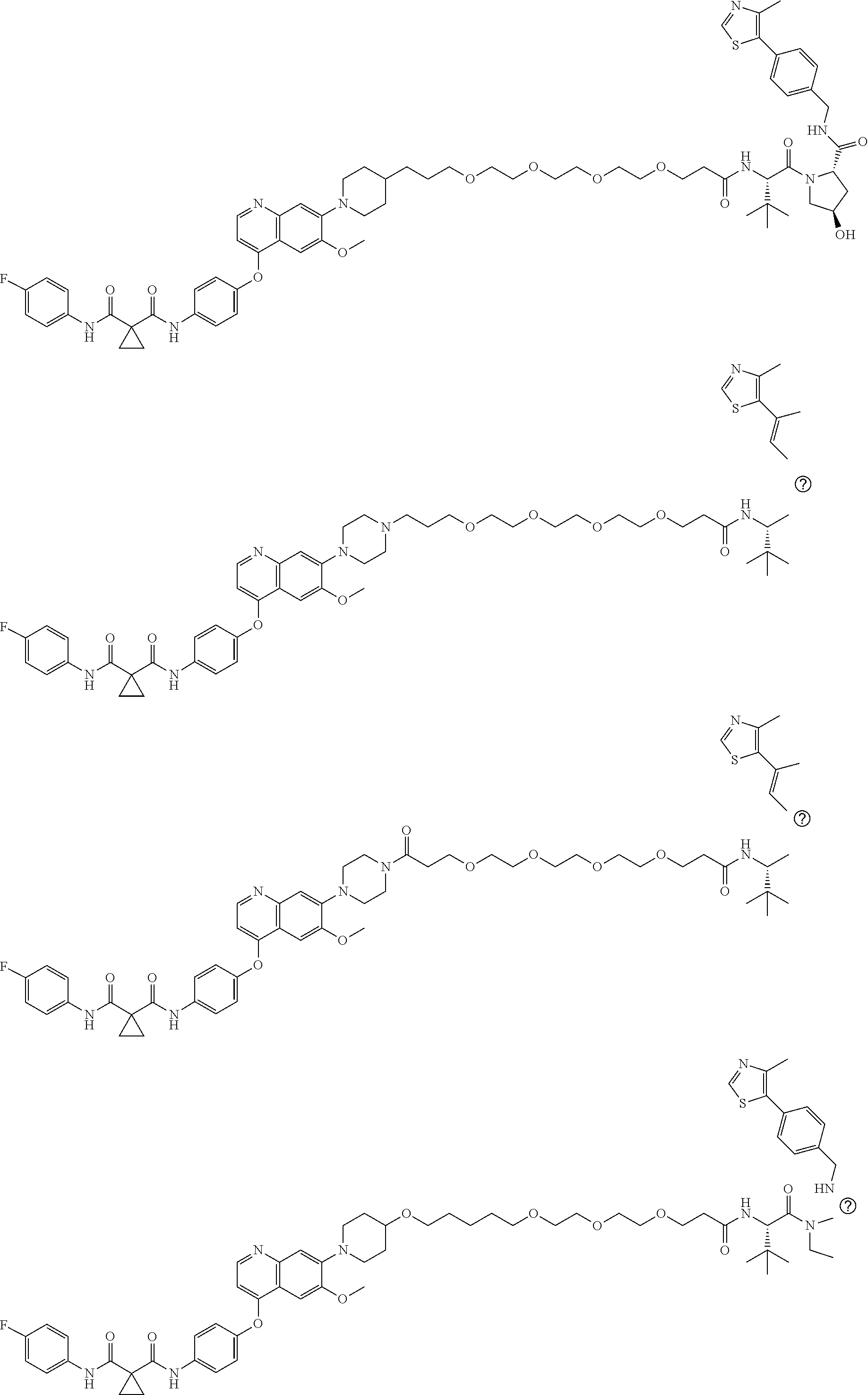

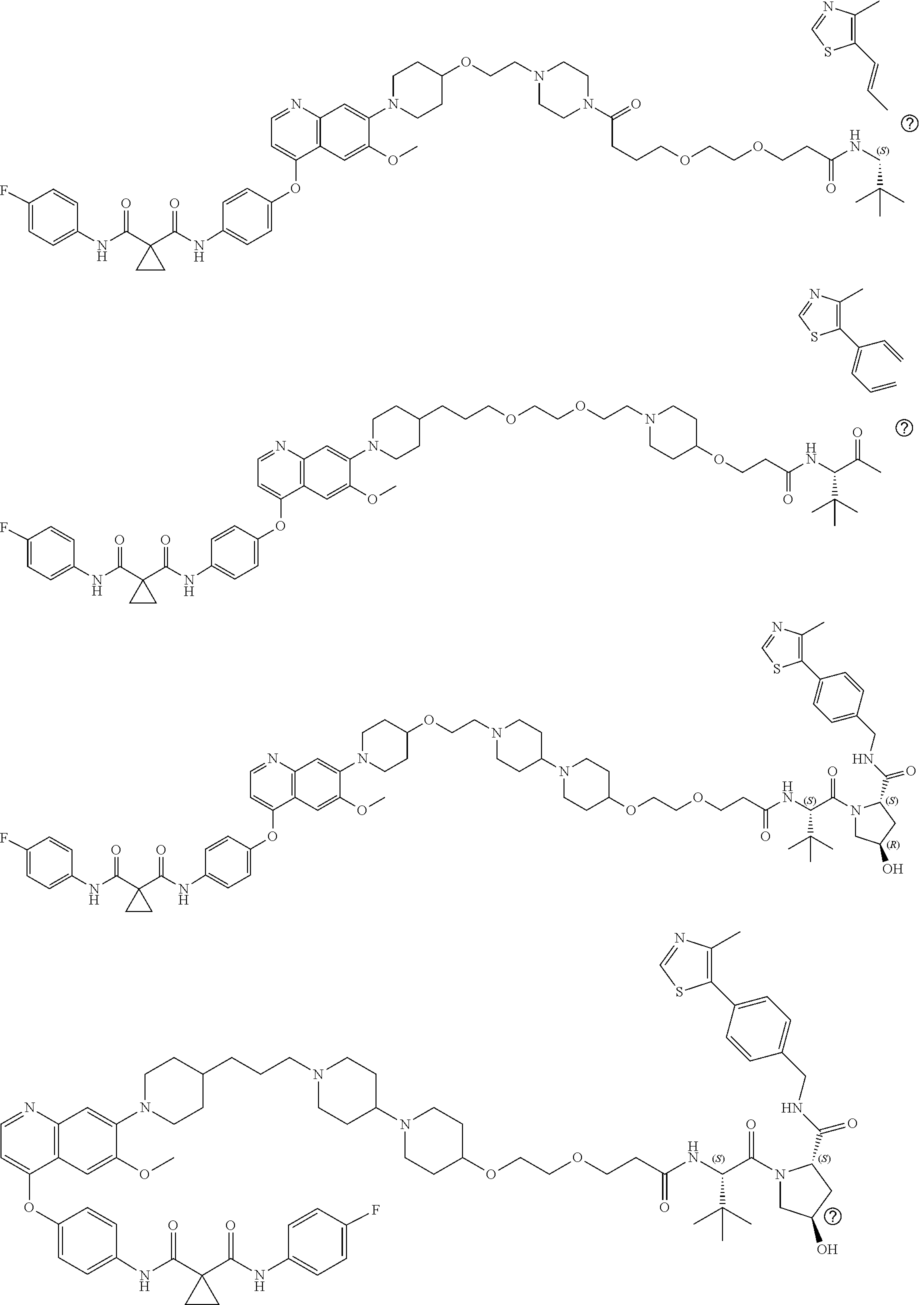

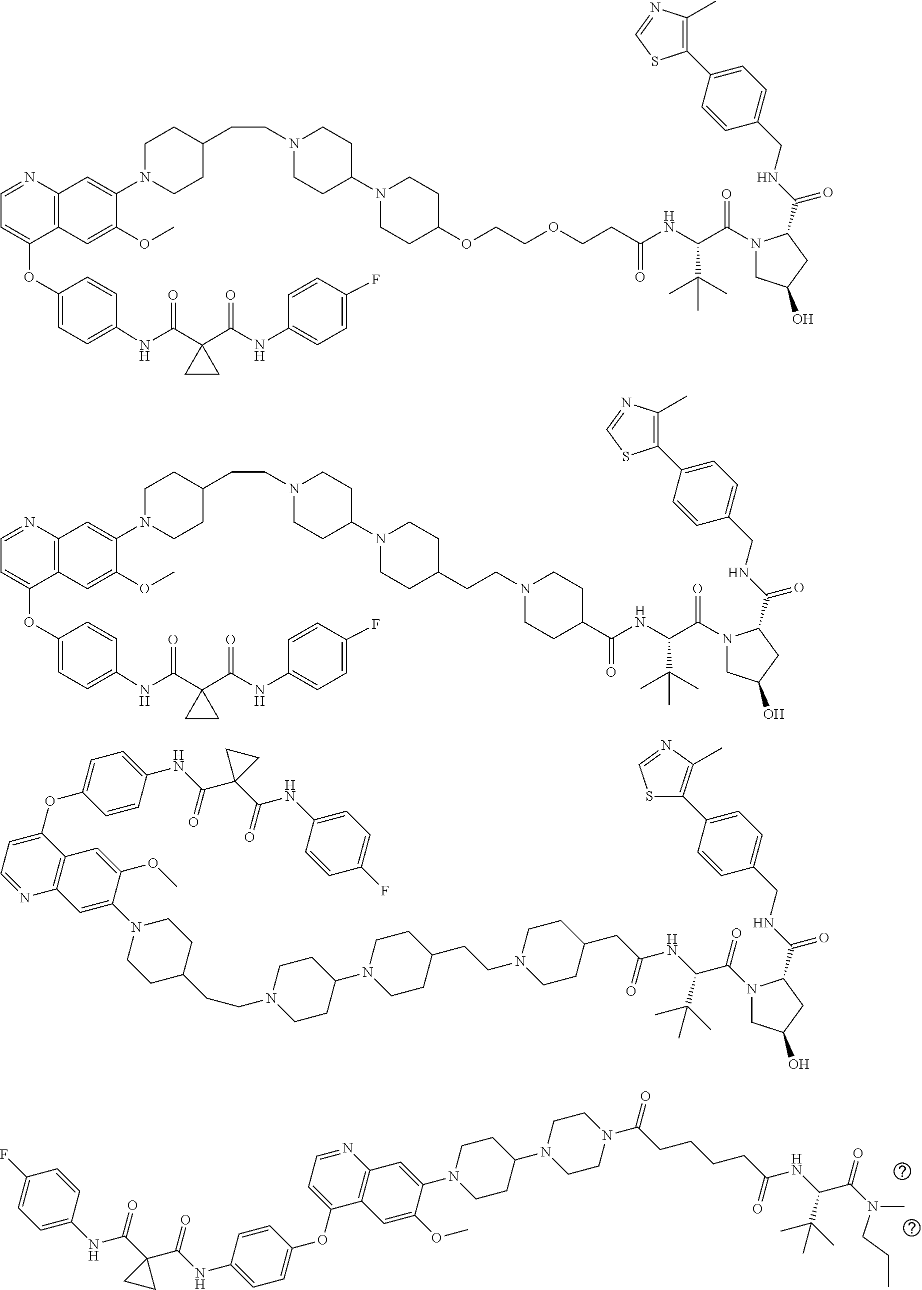

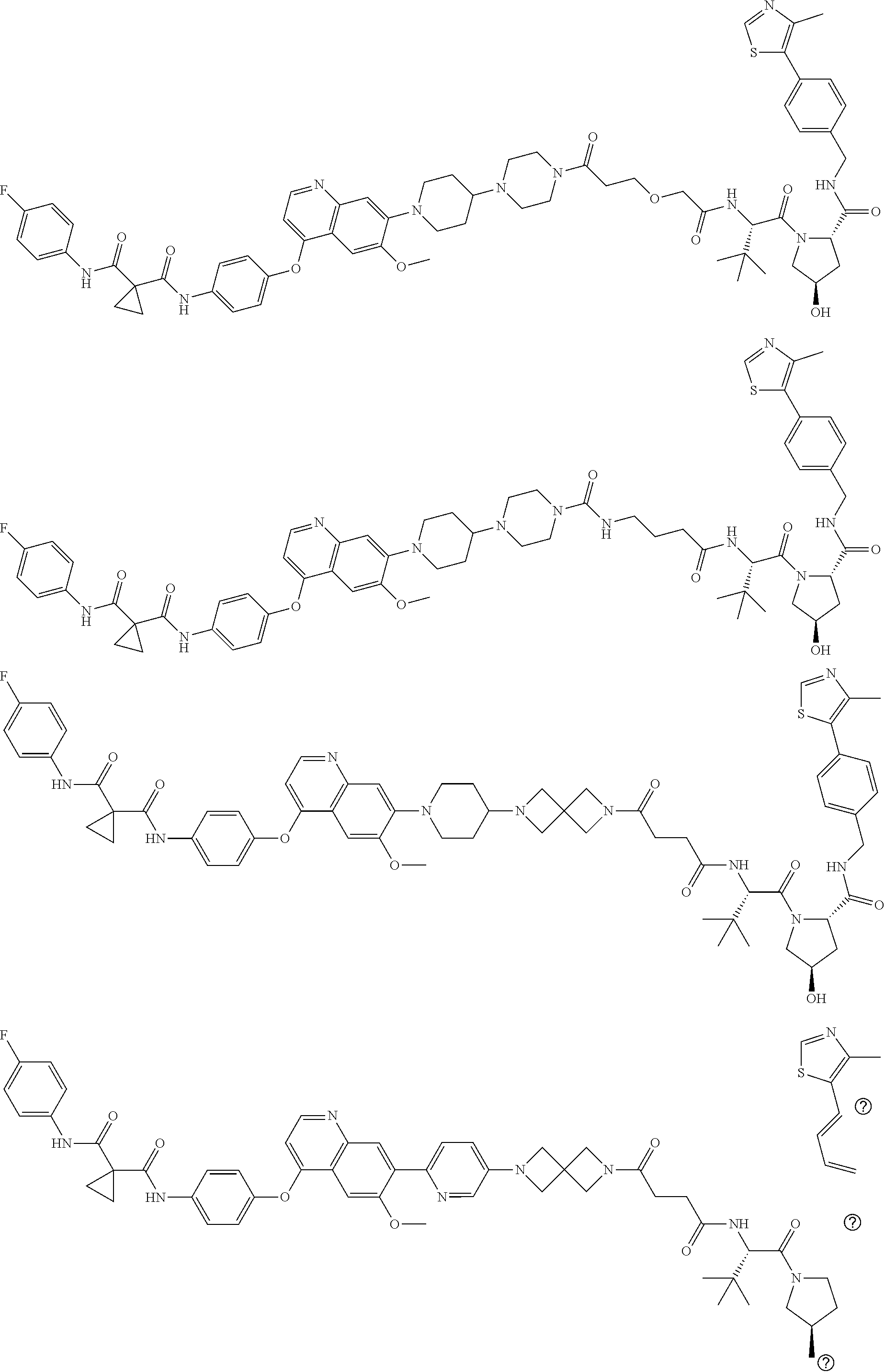

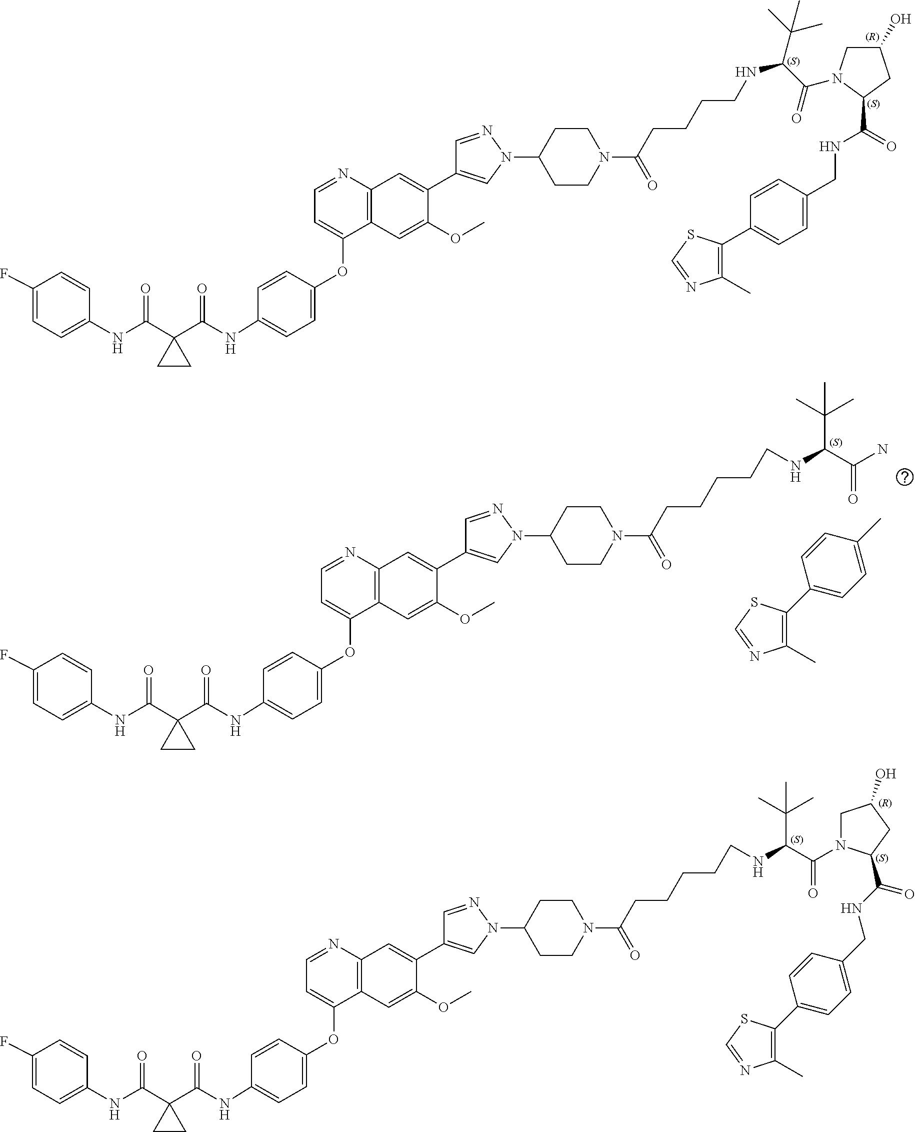

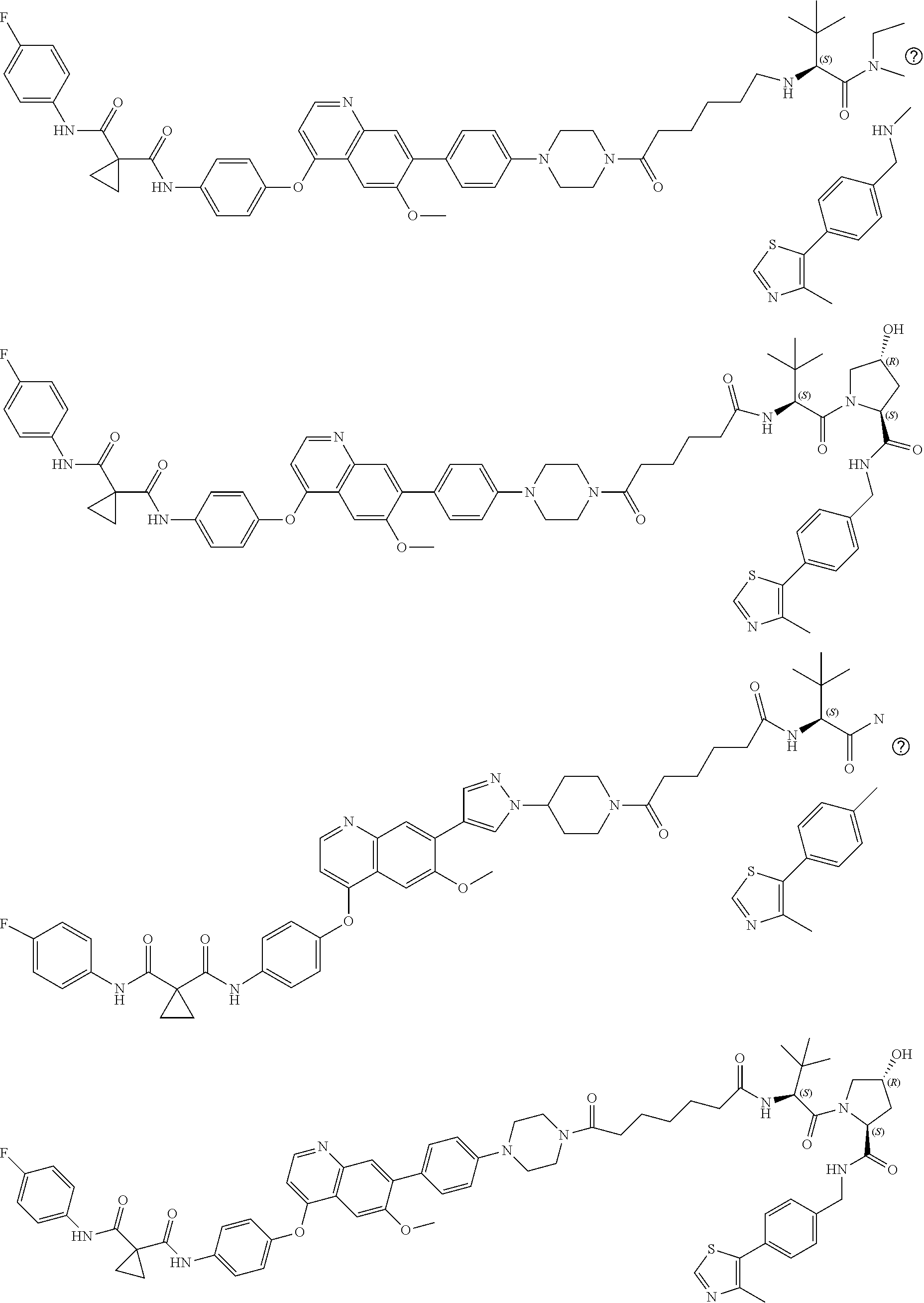

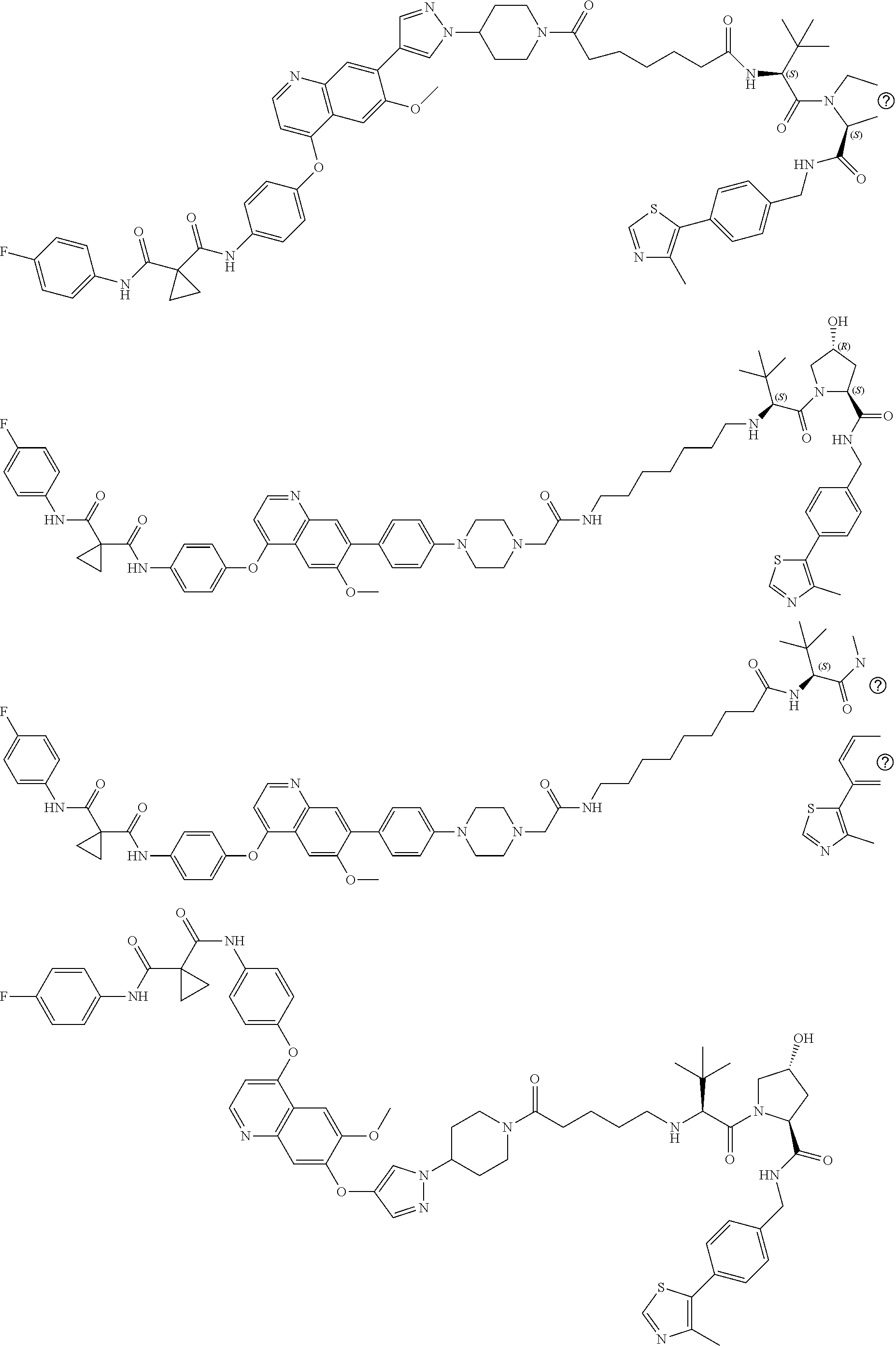

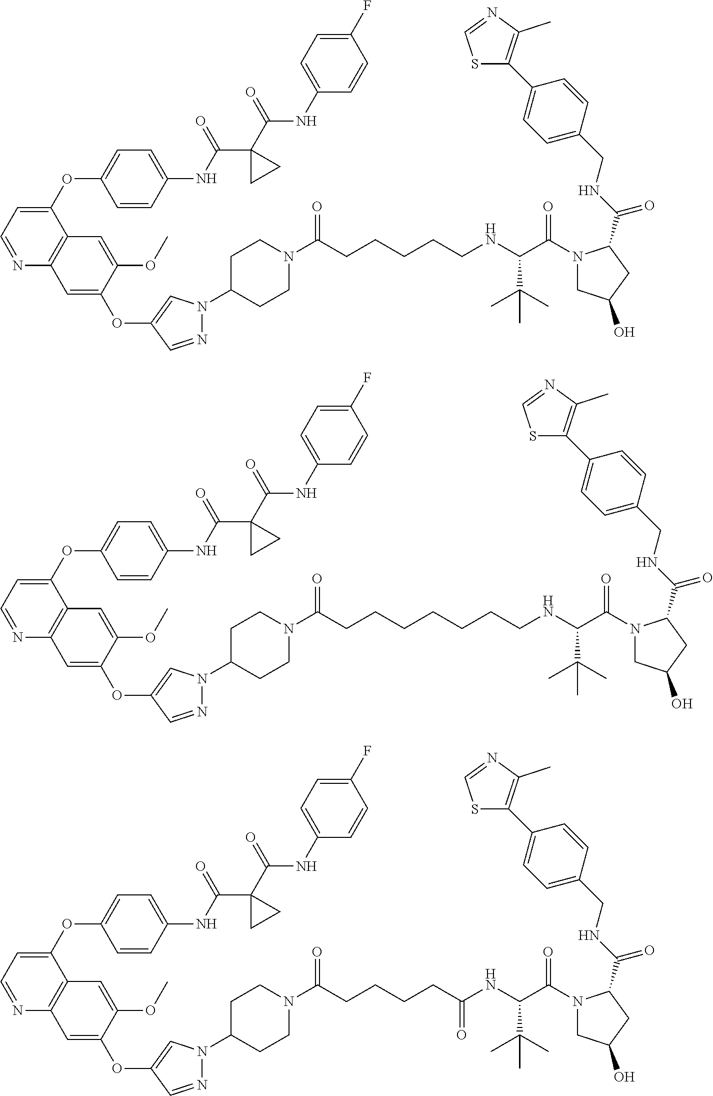 [SMILES: COc1cc2c(Oc3ccc(NC(=O)C4(C(=O)Nc5ccc(F)cc5)CC4)cc3)ccnc2cc1Oc1cnn(C2CCN(C(=O)CCCCCN[C@H](C(=O)N3C[C@H](O)C[C@H]3C(=O)NCc3ccc(-c4scnc4C)cc3)C(C)(C)C)CC2)c1 ; COc1cc2c(Oc3ccc(NC(=O)C4(C(=O)Nc5ccc(F)cc5)CC4)cc3)ccnc2cc1Oc1cnn(C2CCN(C(=O)CCCCCCCN[C@H](C(=O)N3C[C@H](O)C[C@H]3C(=O)NCc3ccc(-c4scnc4C)cc3)C(C)(C)C)CC2)c1 ; COc1cc2c(Oc3ccc(NC(=O)C4(C(=O)Nc5ccc(F)cc5)CC4)cc3)ccnc2cc1Oc1cnn(C2CCN(C(=O)CCCCC(=O)N[C@H](C(=O)N3C[C@H](O)C[C@H]3C(=O)NCc3ccc(-c4scnc4C)cc3)C(C)(C)C)CC2)c1]

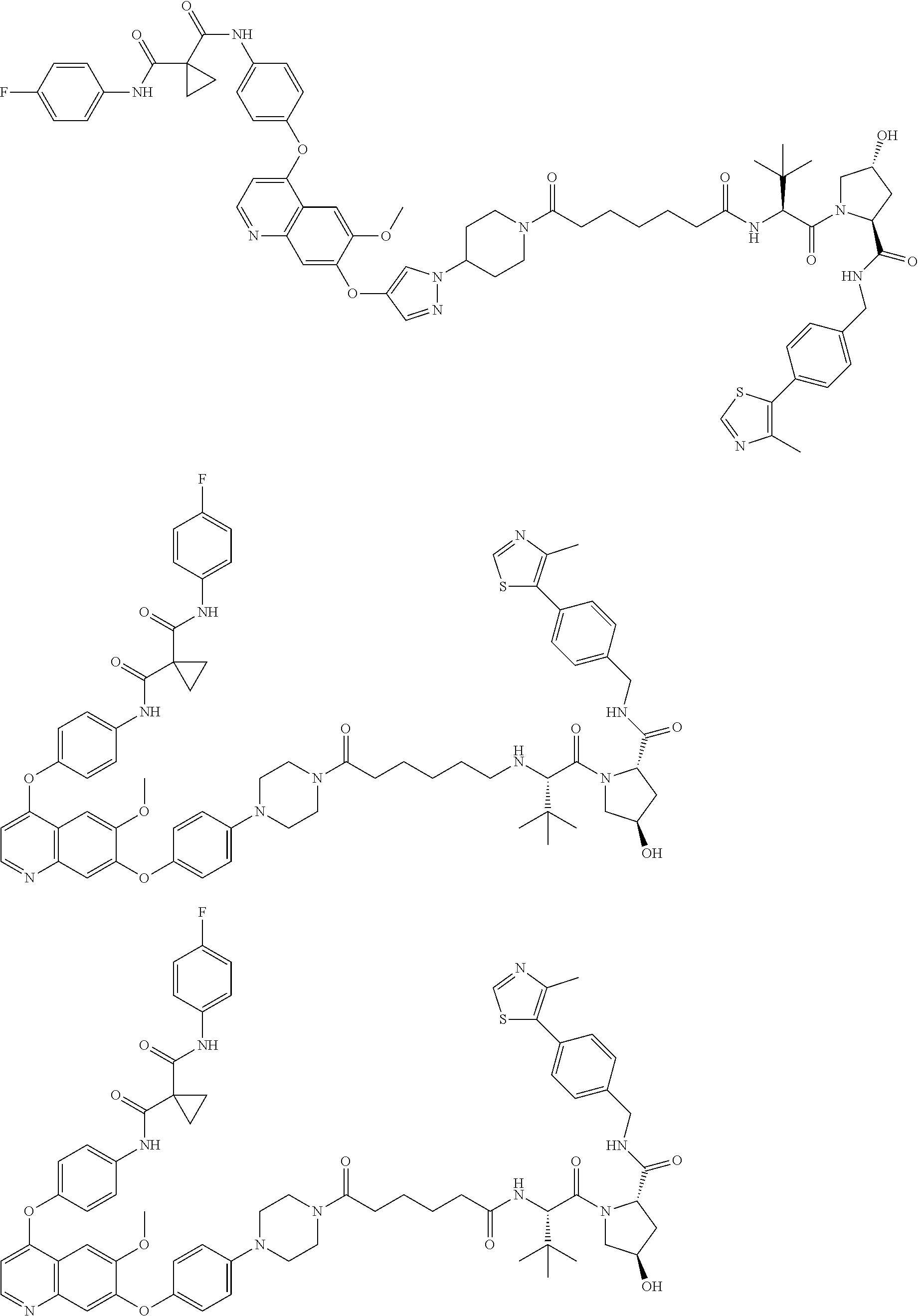

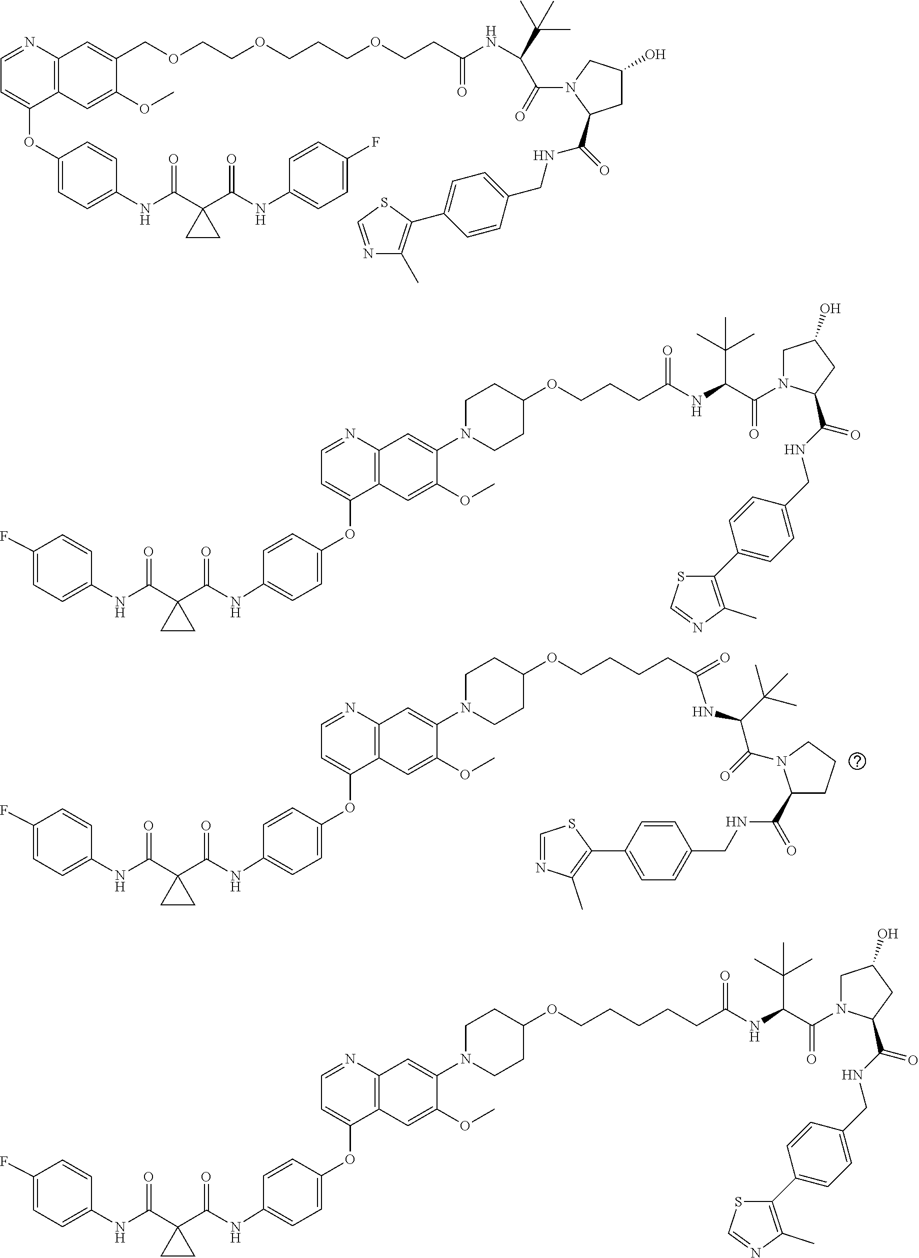

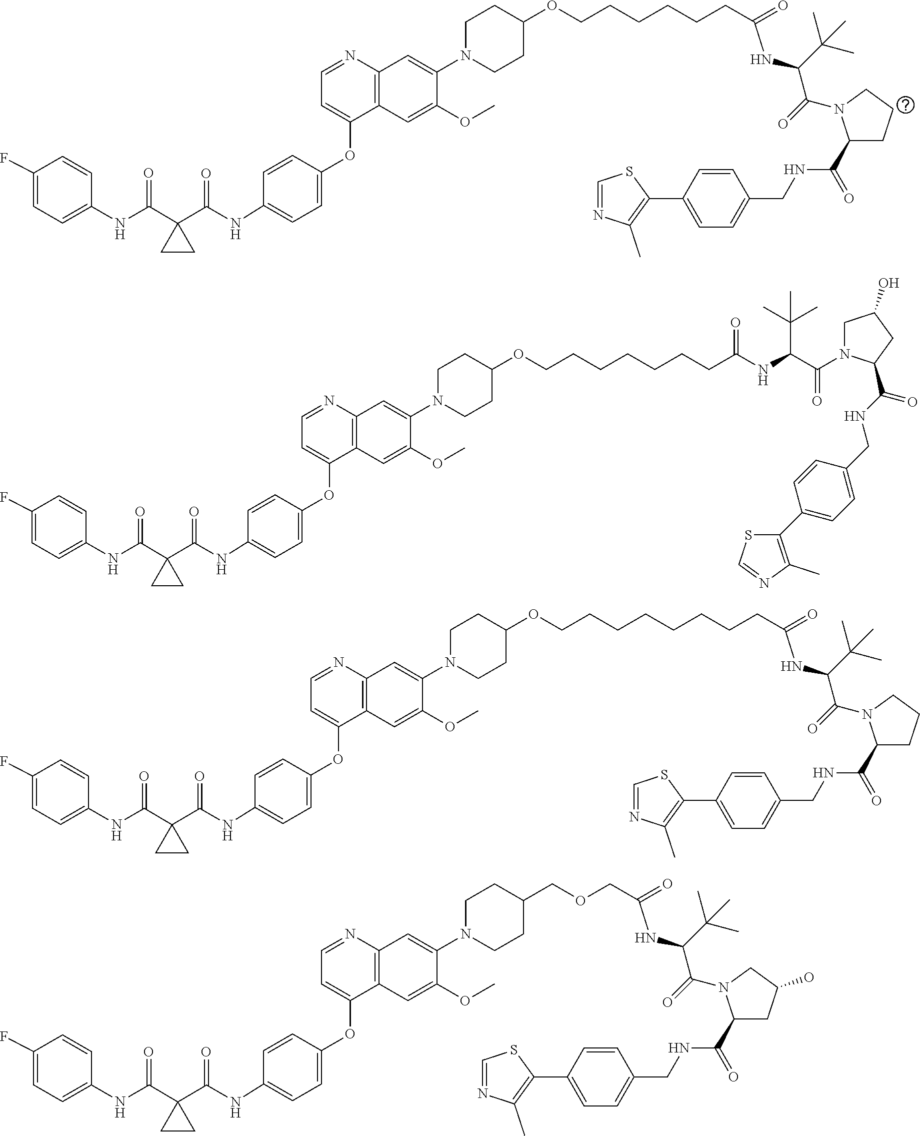

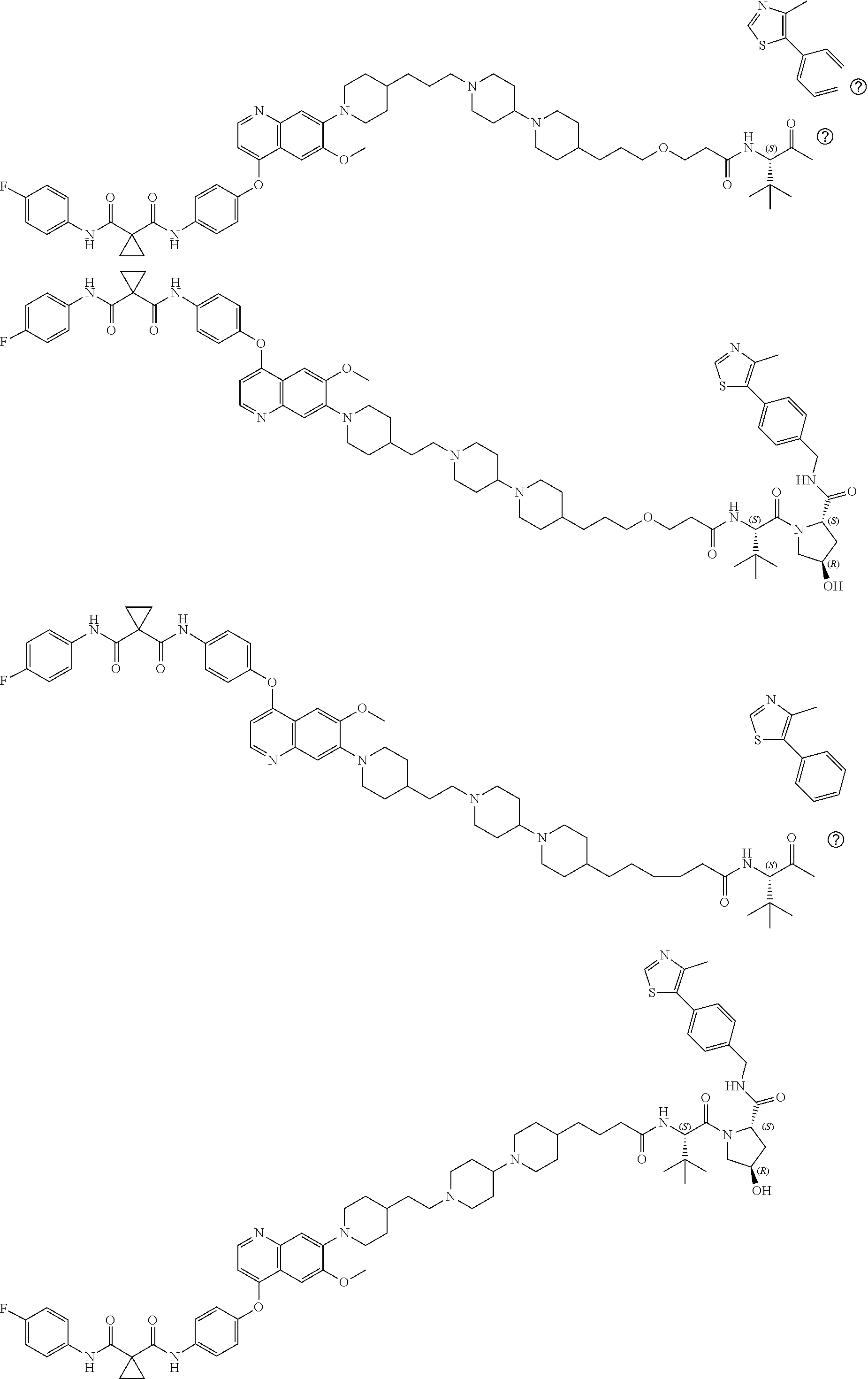

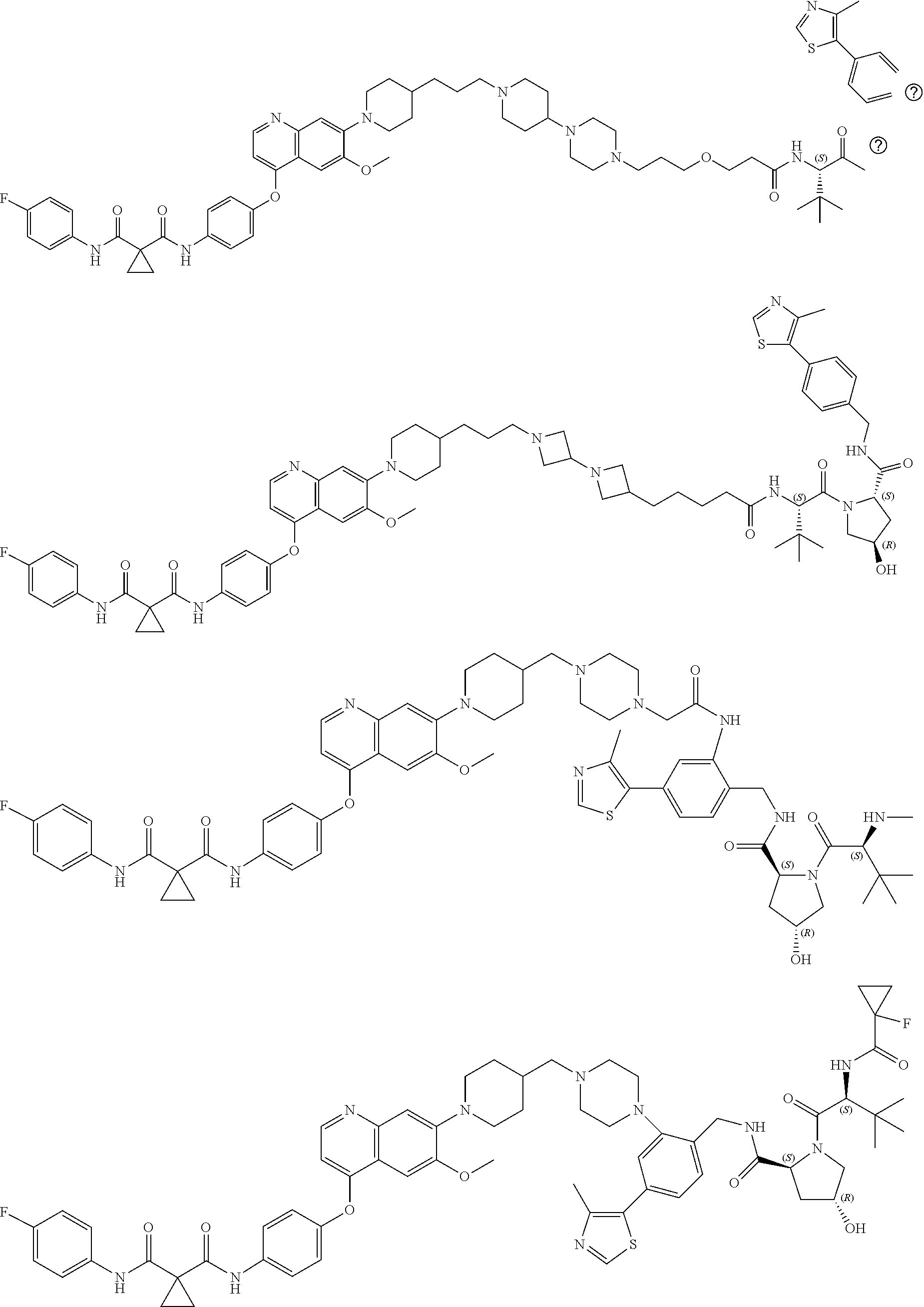

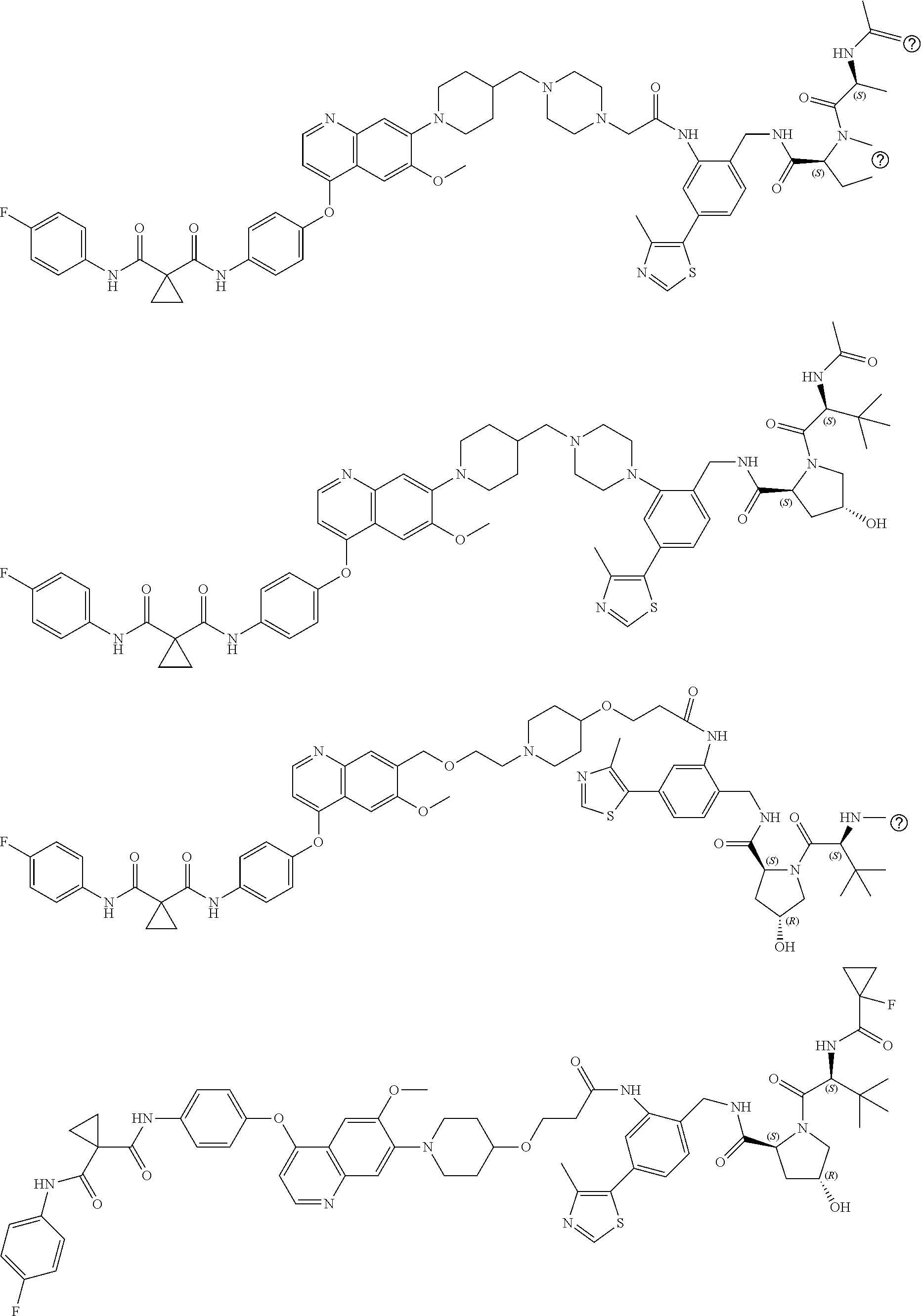

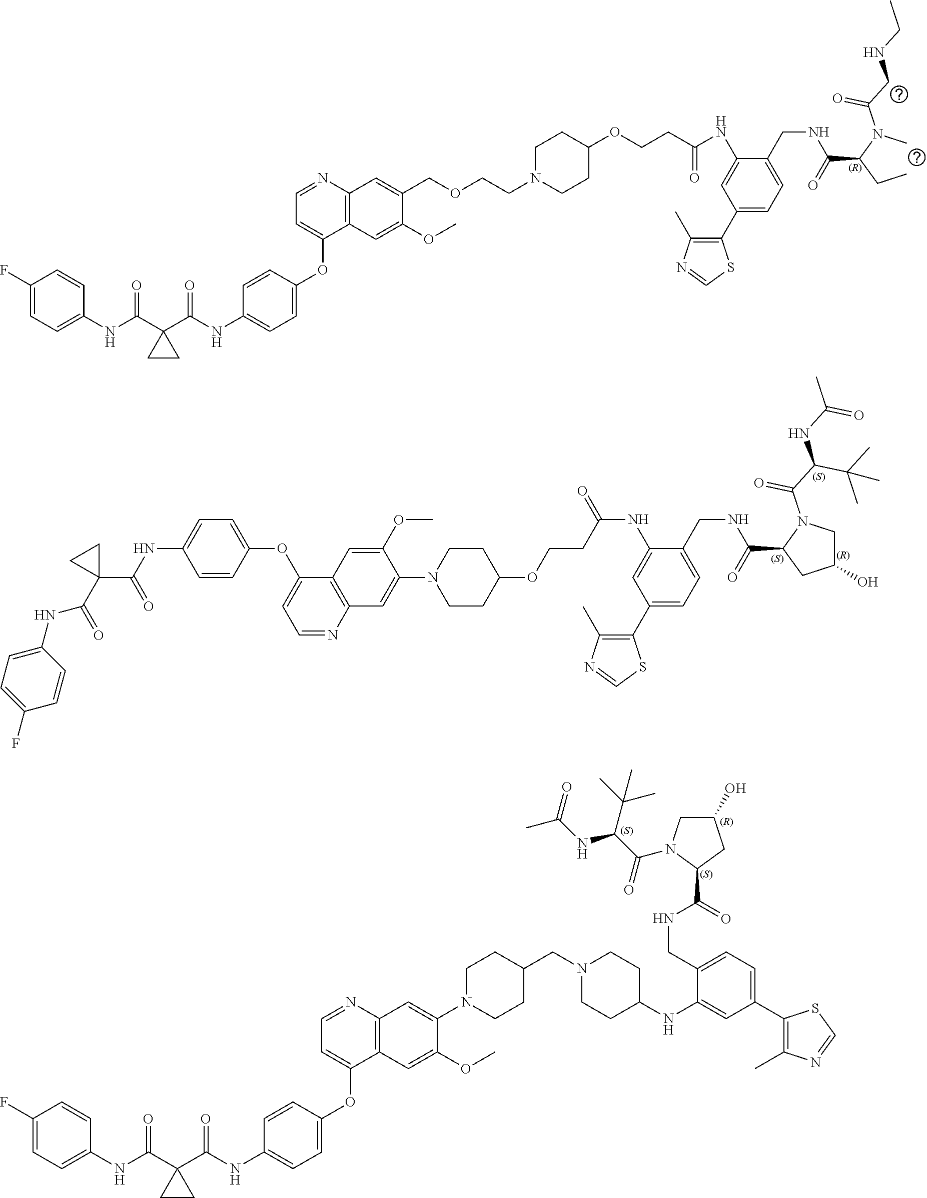

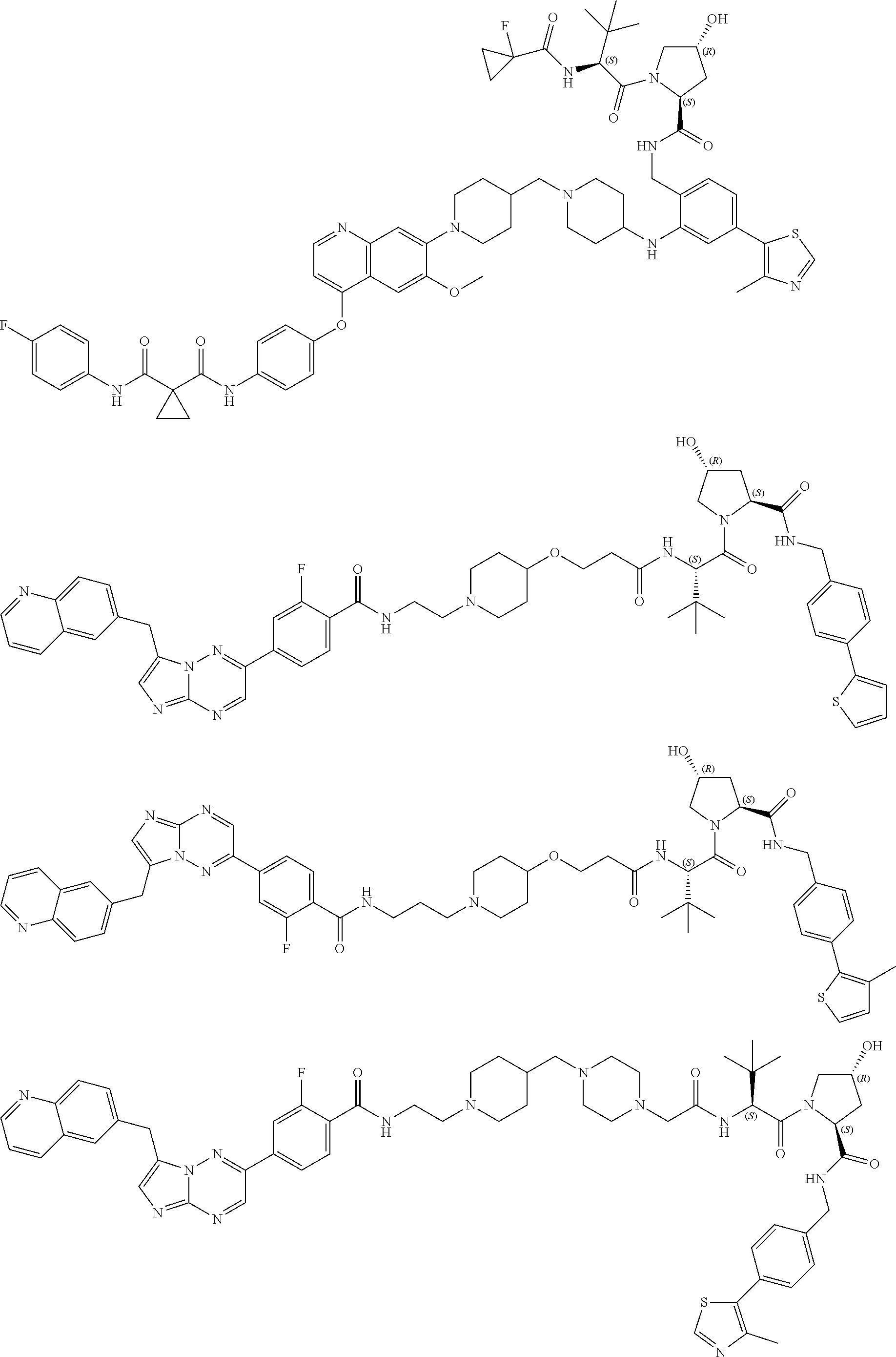

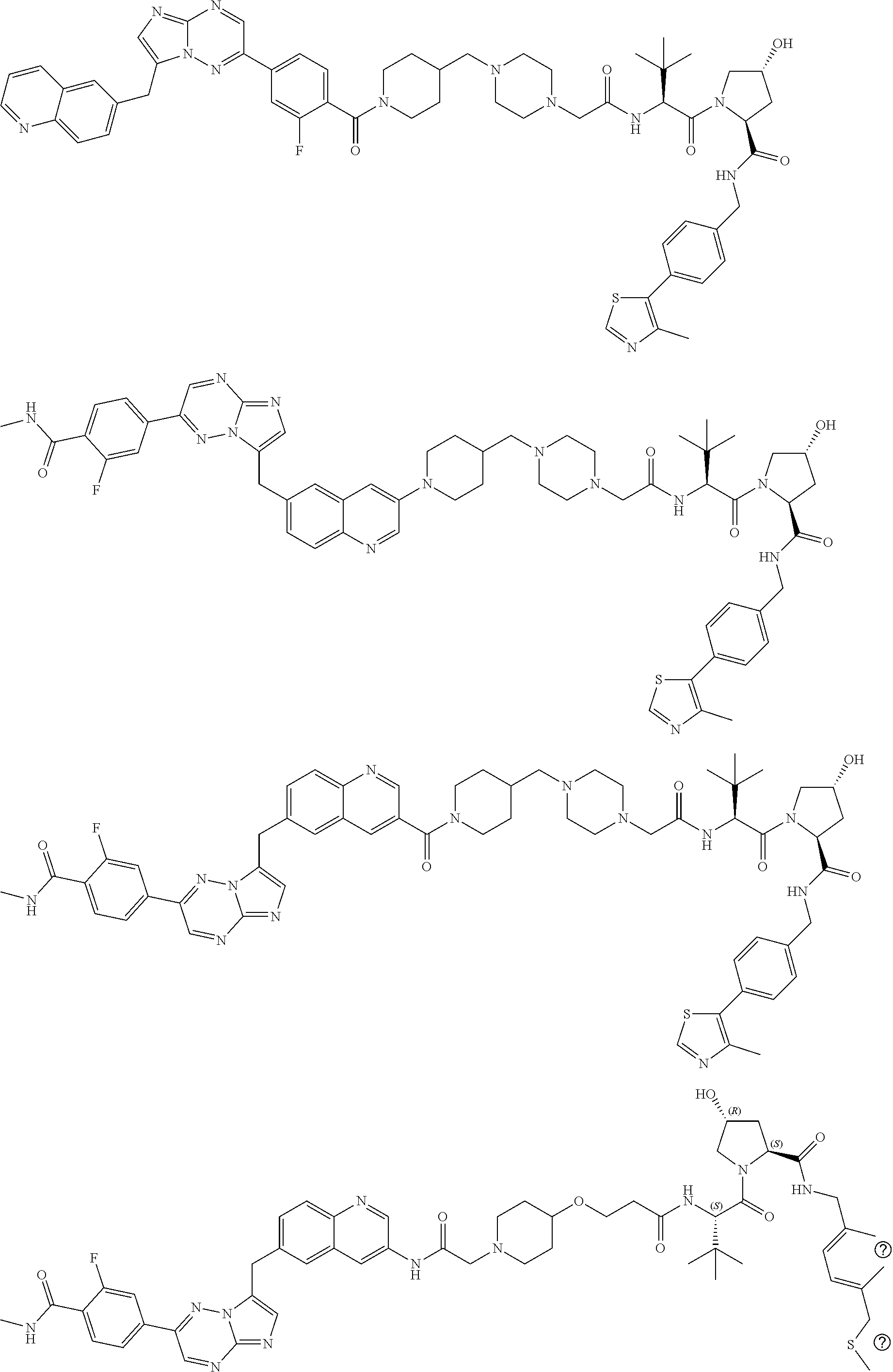

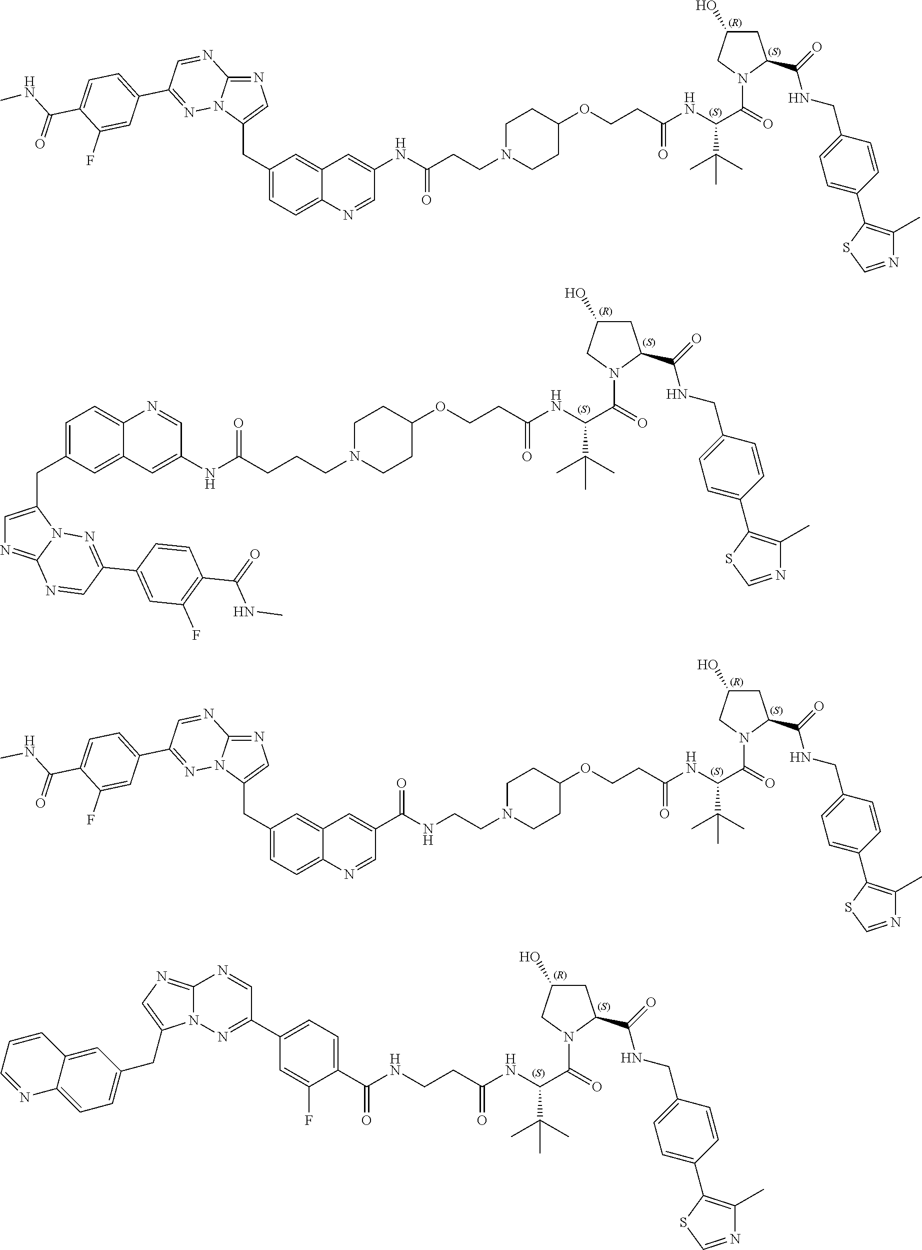

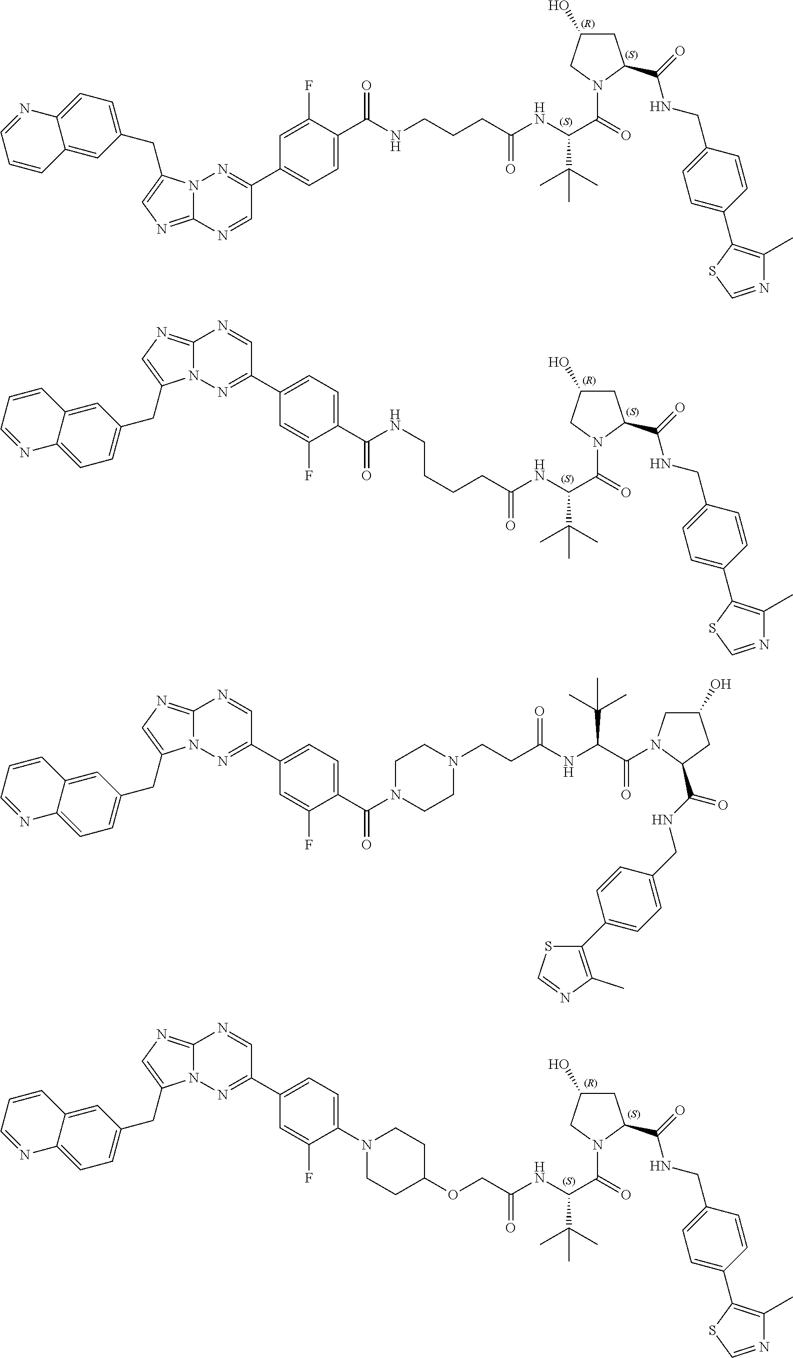

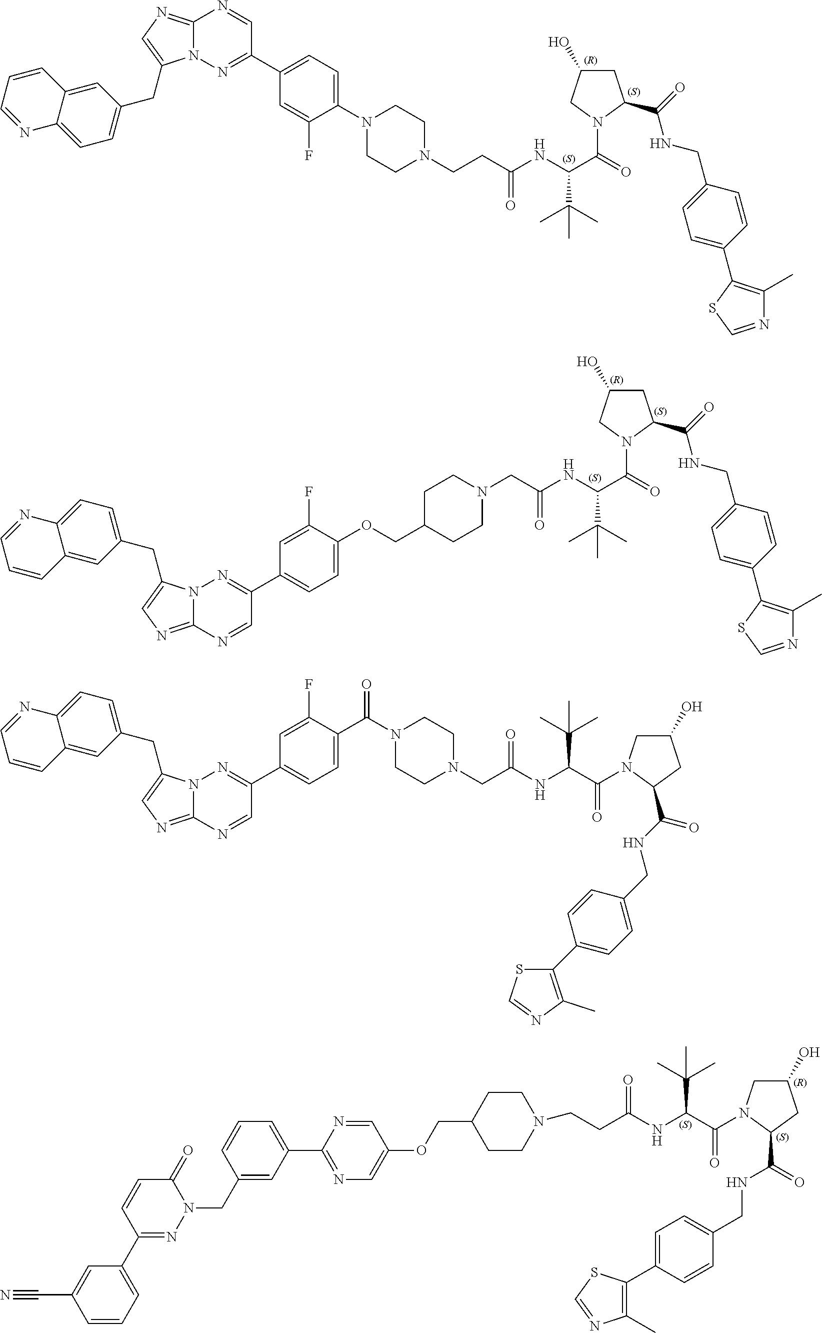

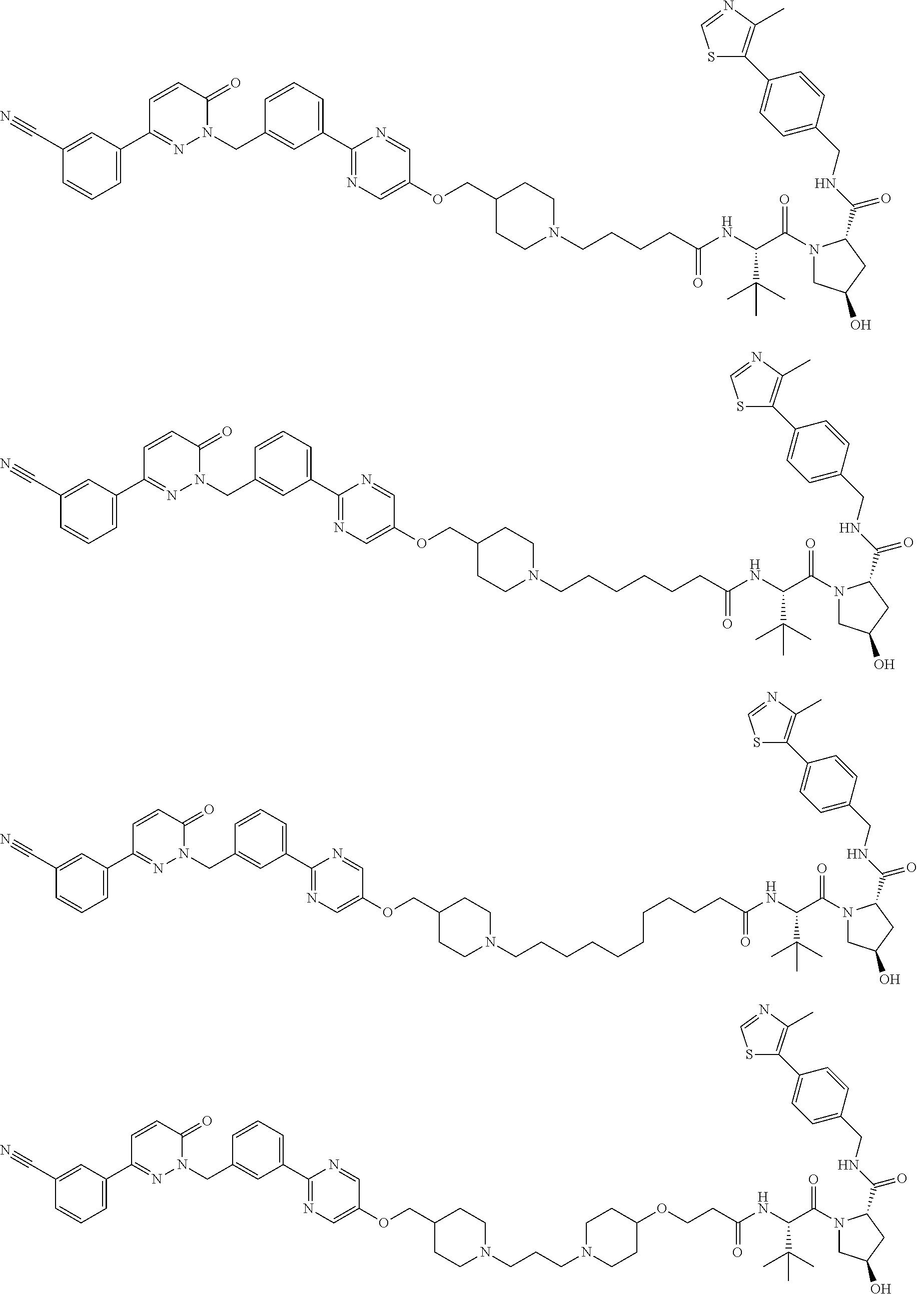

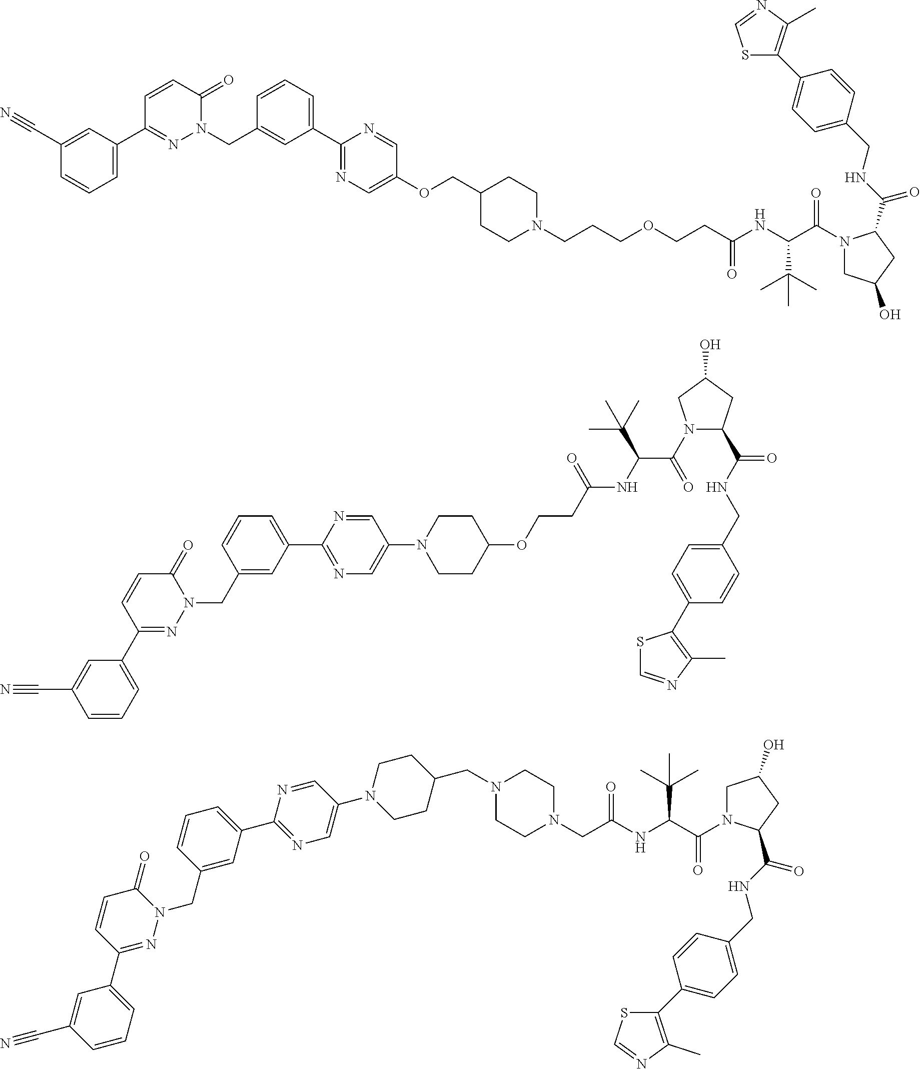 [SMILES: Cc1ncsc1-c1ccc(CNC(=O)[C@@H]2C[C@@H](O)CN2C(=O)[C@@H](NC(=O)CCOCCCN2CCC(COc3cnc(-c4cccc(Cn5nc(-c6cccc(C#N)c6)ccc5=O)c4)nc3)CC2)C(C)(C)C)cc1 ; Cc1ncsc1-c1ccc(CNC(=O)[C@@H]2C[C@@H](O)CN2C(=O)[C@@H](NC(=O)CCOC2CCN(c3cnc(-c4cccc(Cn5nc(-c6cccc(C#N)c6)ccc5=O)c4)nc3)CC2)C(C)(C)C)cc1 ; Cc1ncsc1-c1ccc(CNC(=O)[C@@H]2C[C@@H](O)CN2C(=O)[C@@H](NC(=O)CN2CCN(CC3CCN(c4cnc(-c5cccc(Cn6nc(-c7cccc(C#N)c7)ccc6=O)c5)nc4)CC3)CC2)C(C)(C)C)cc1]

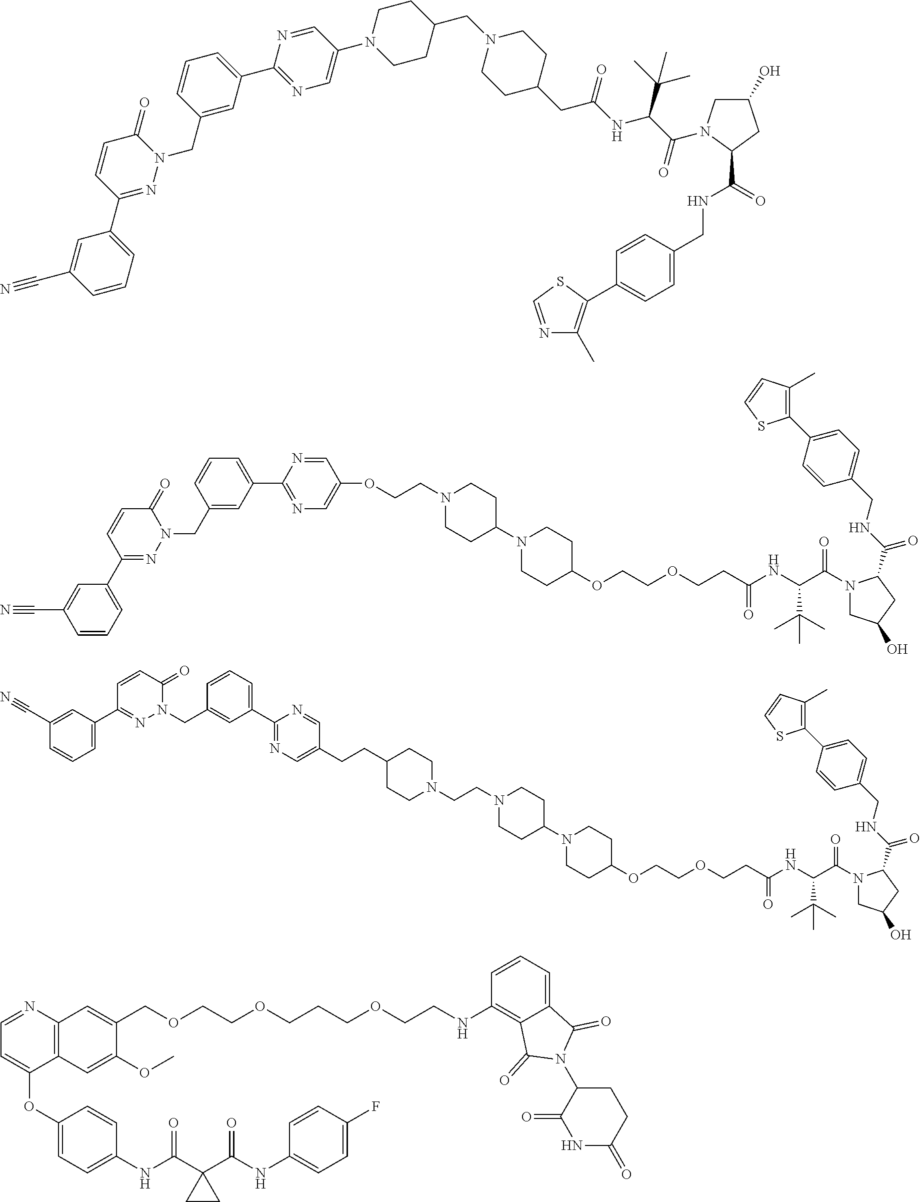

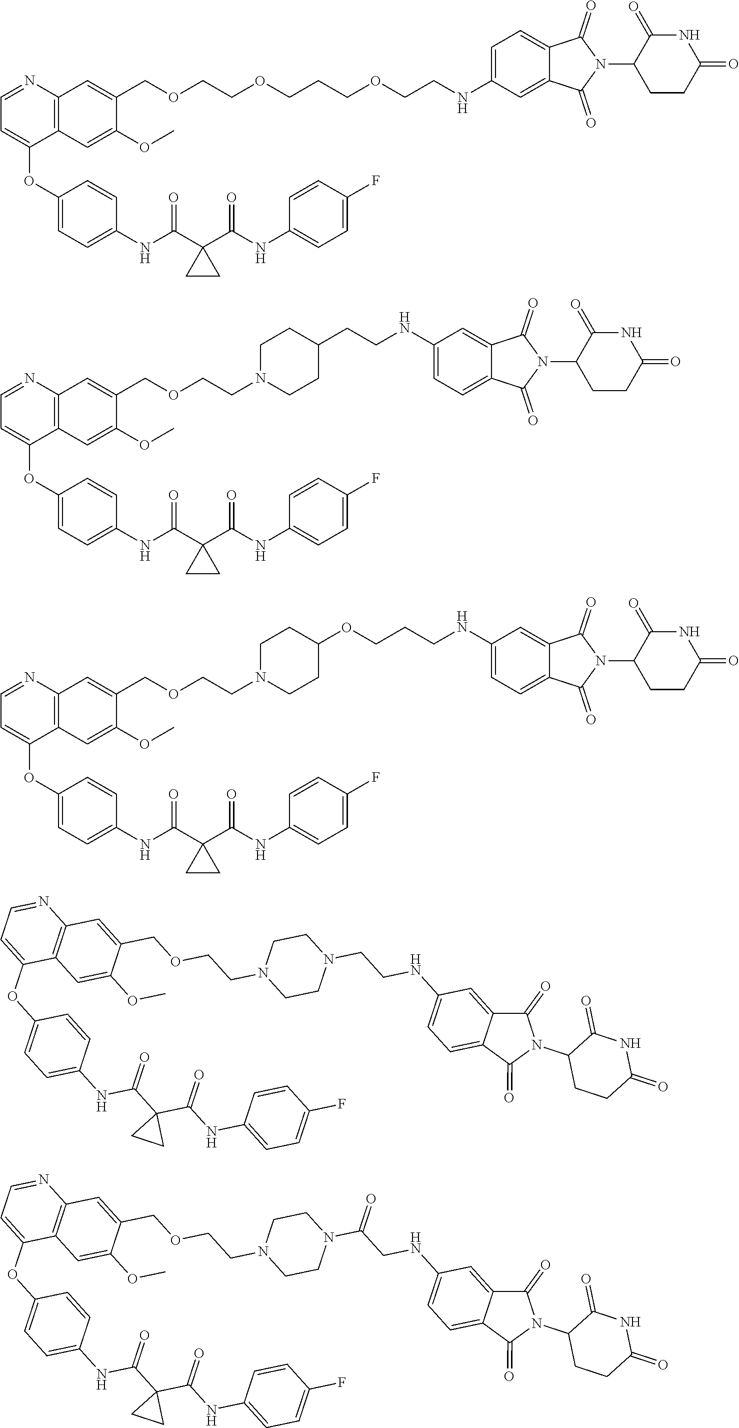

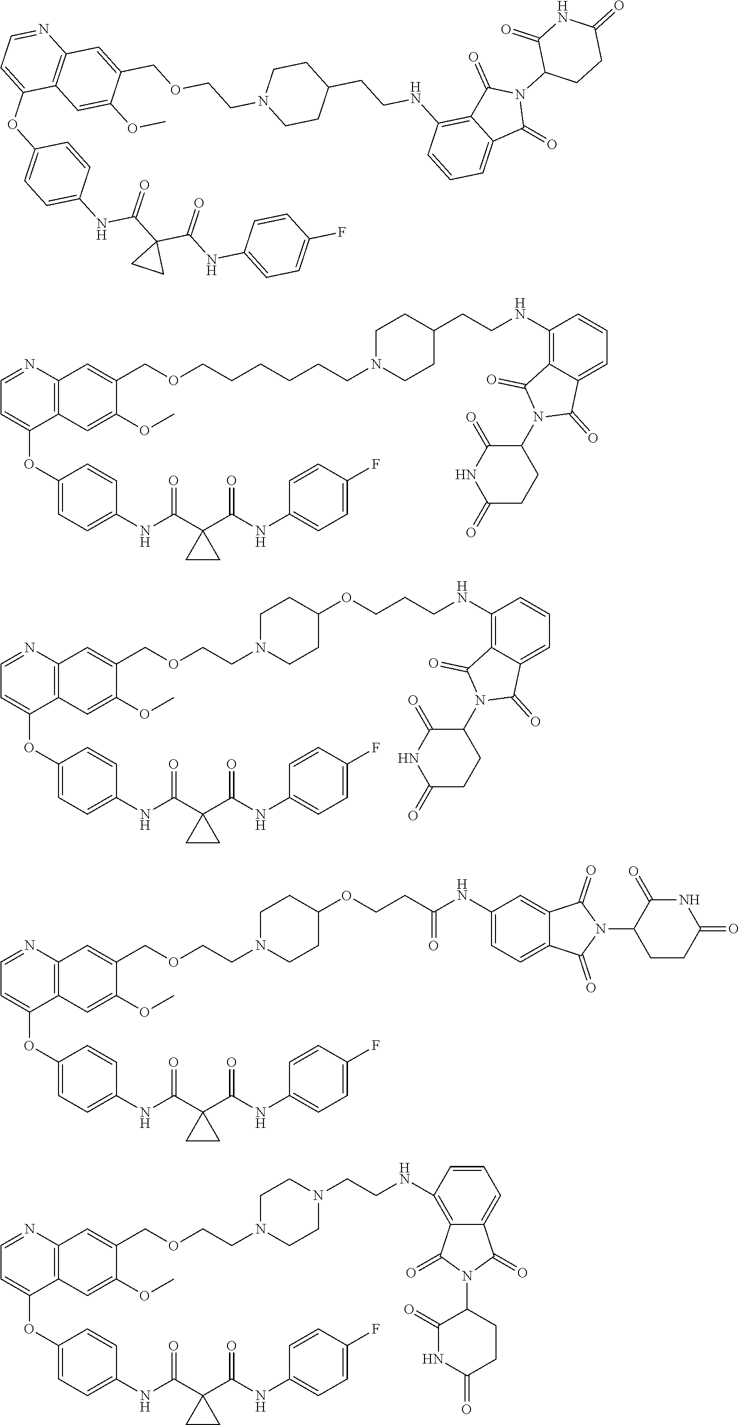

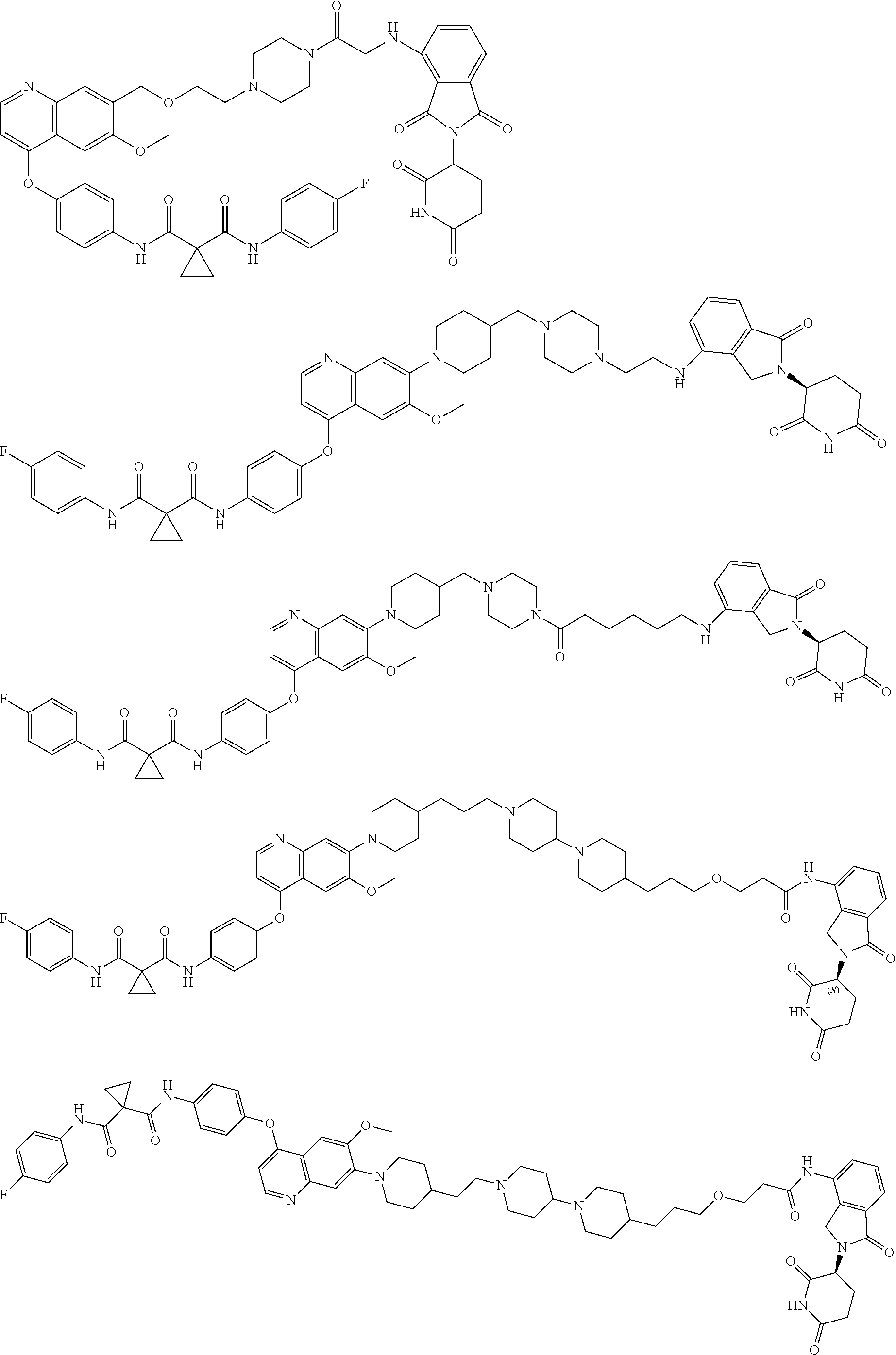

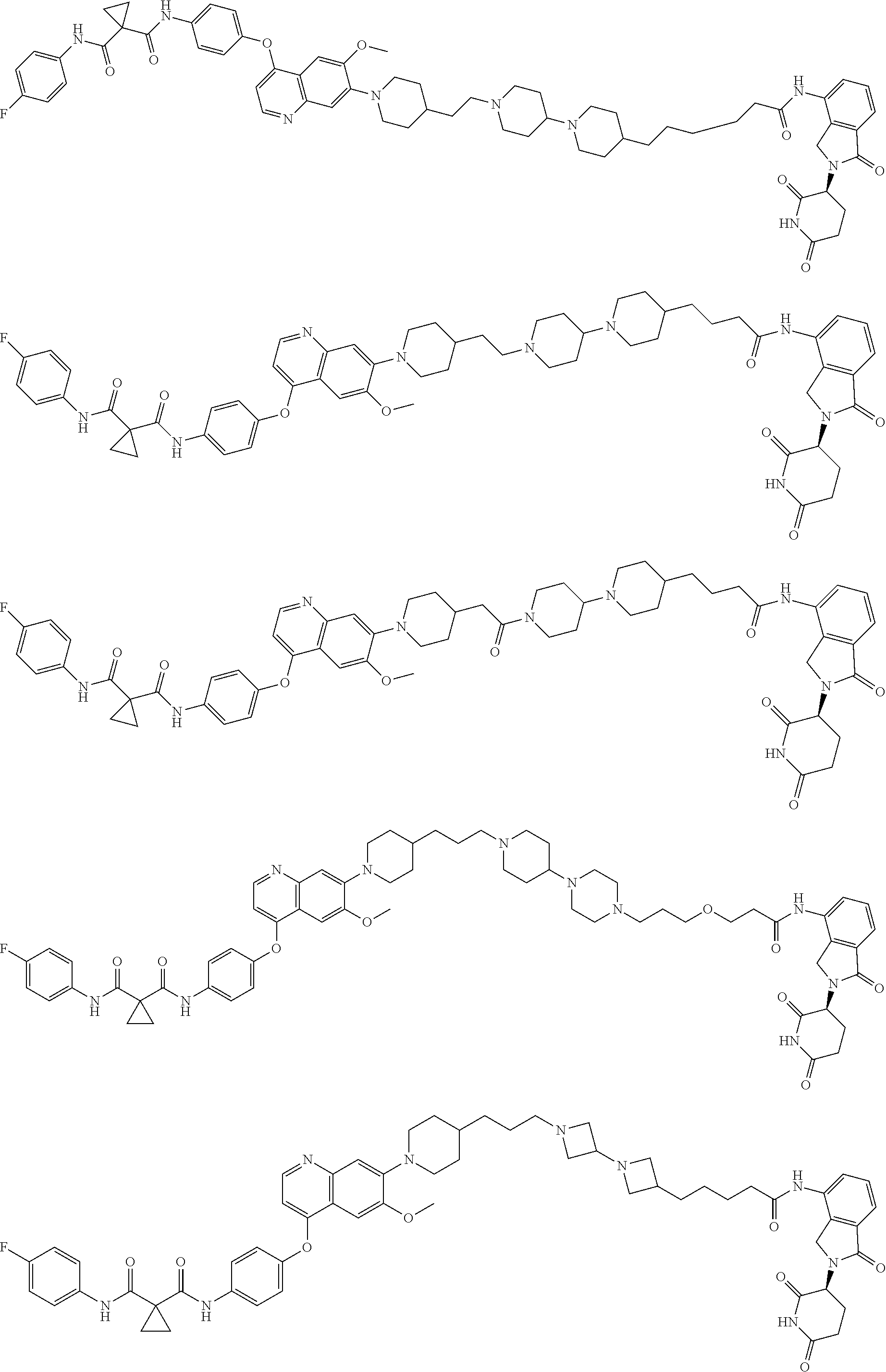

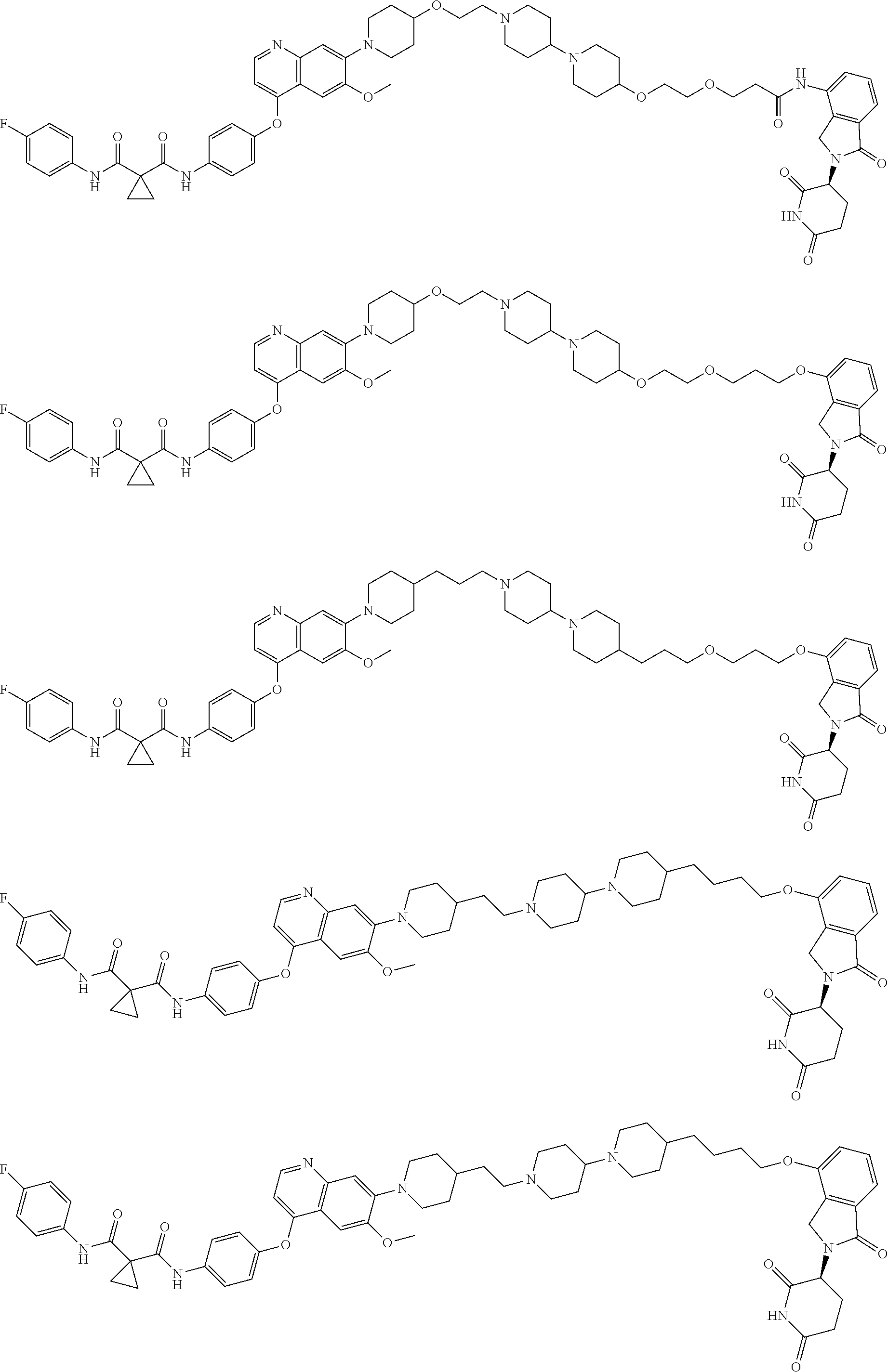

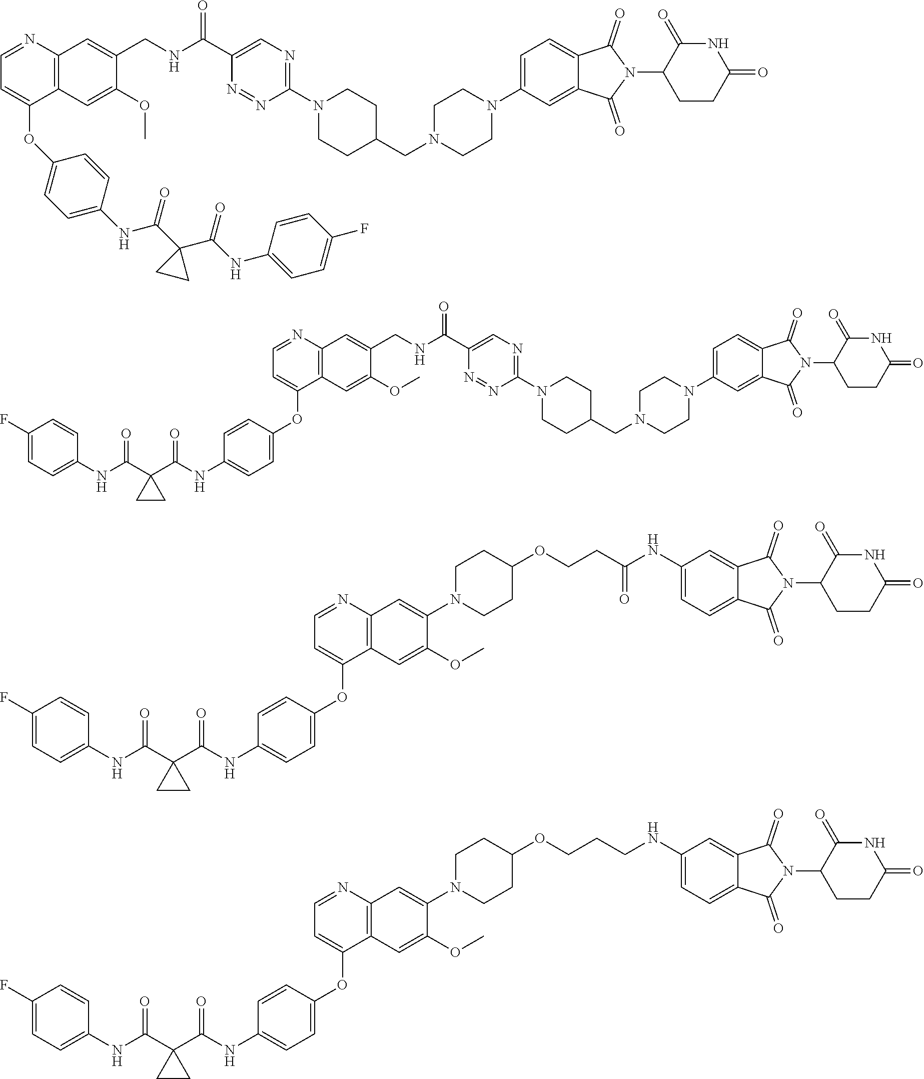

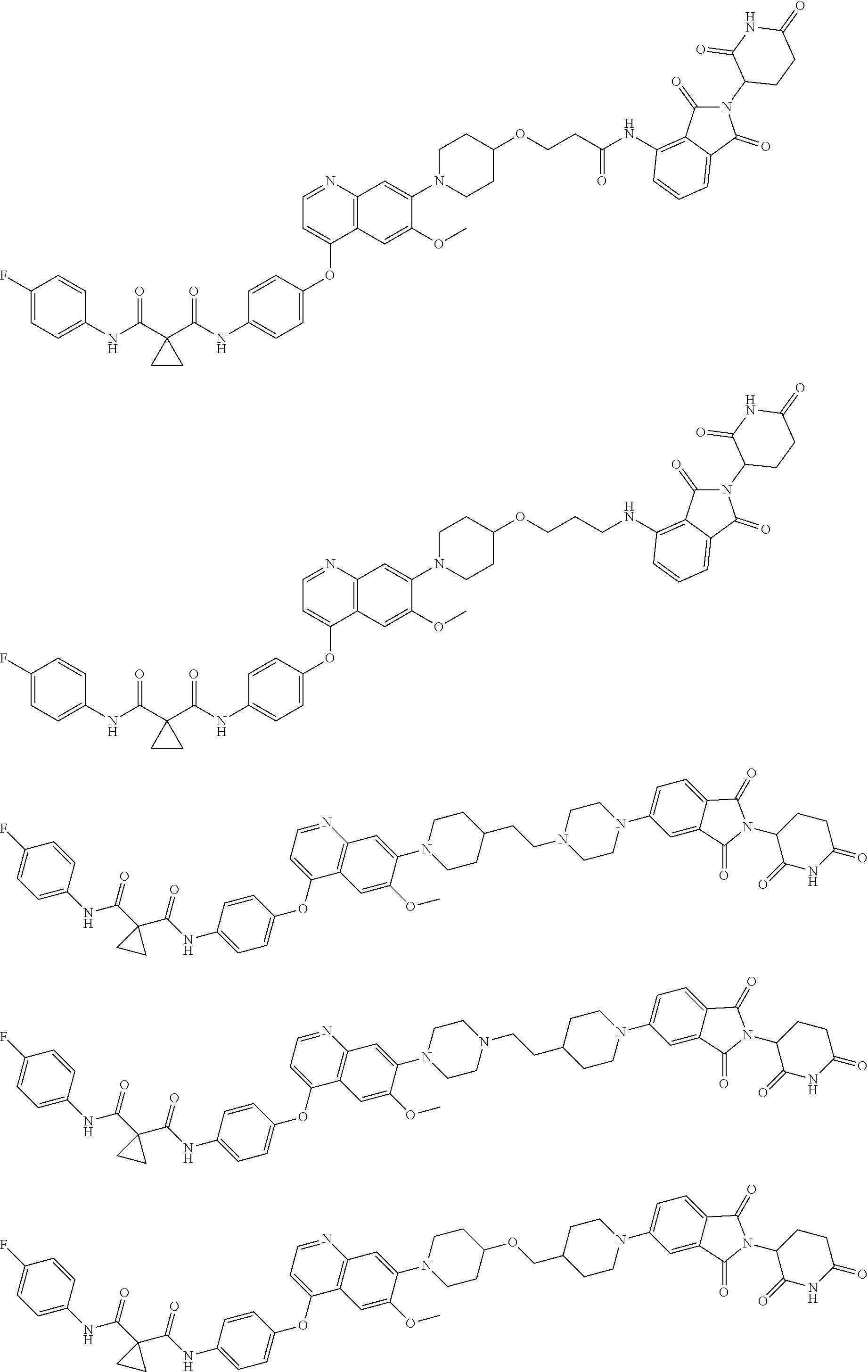

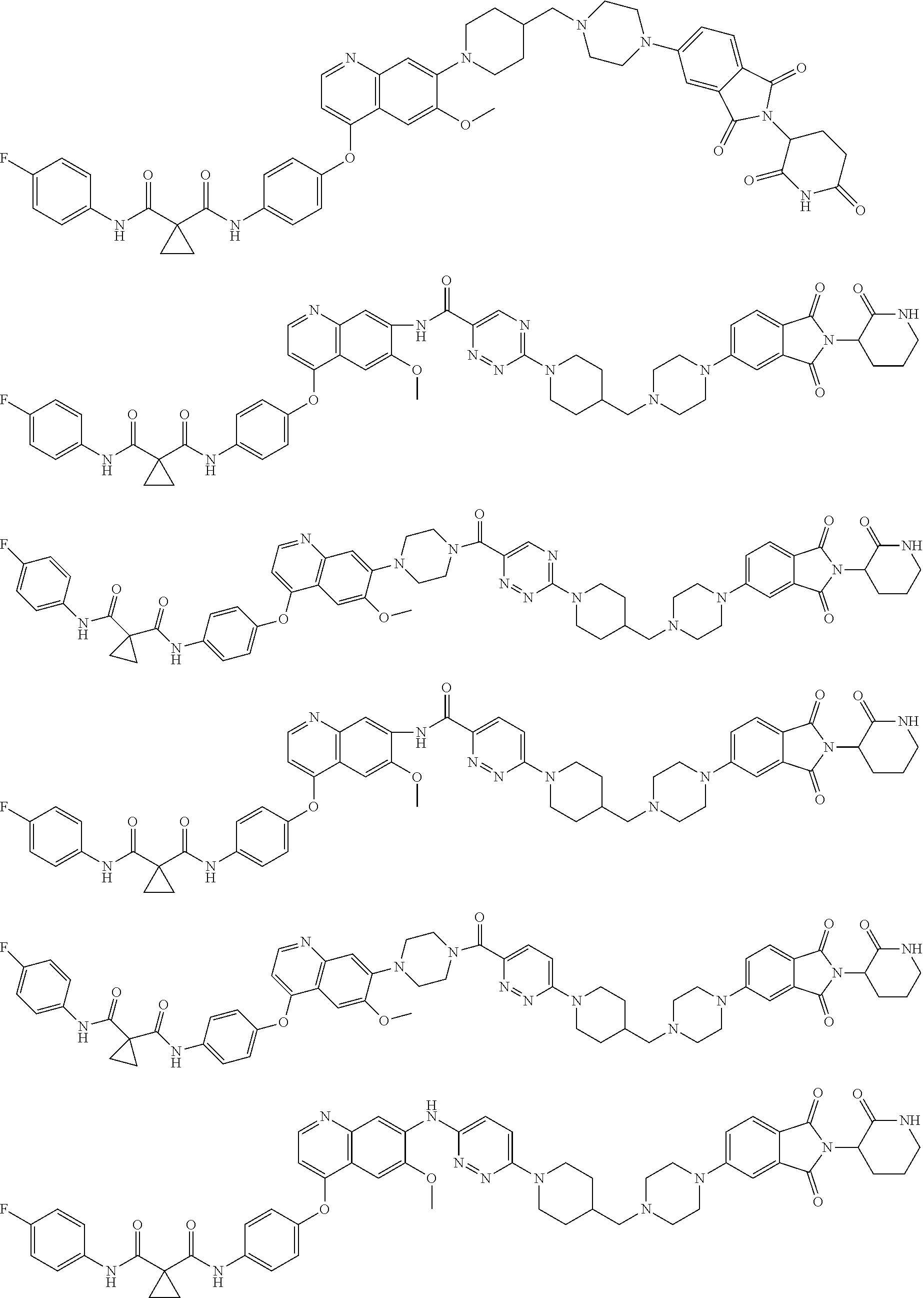

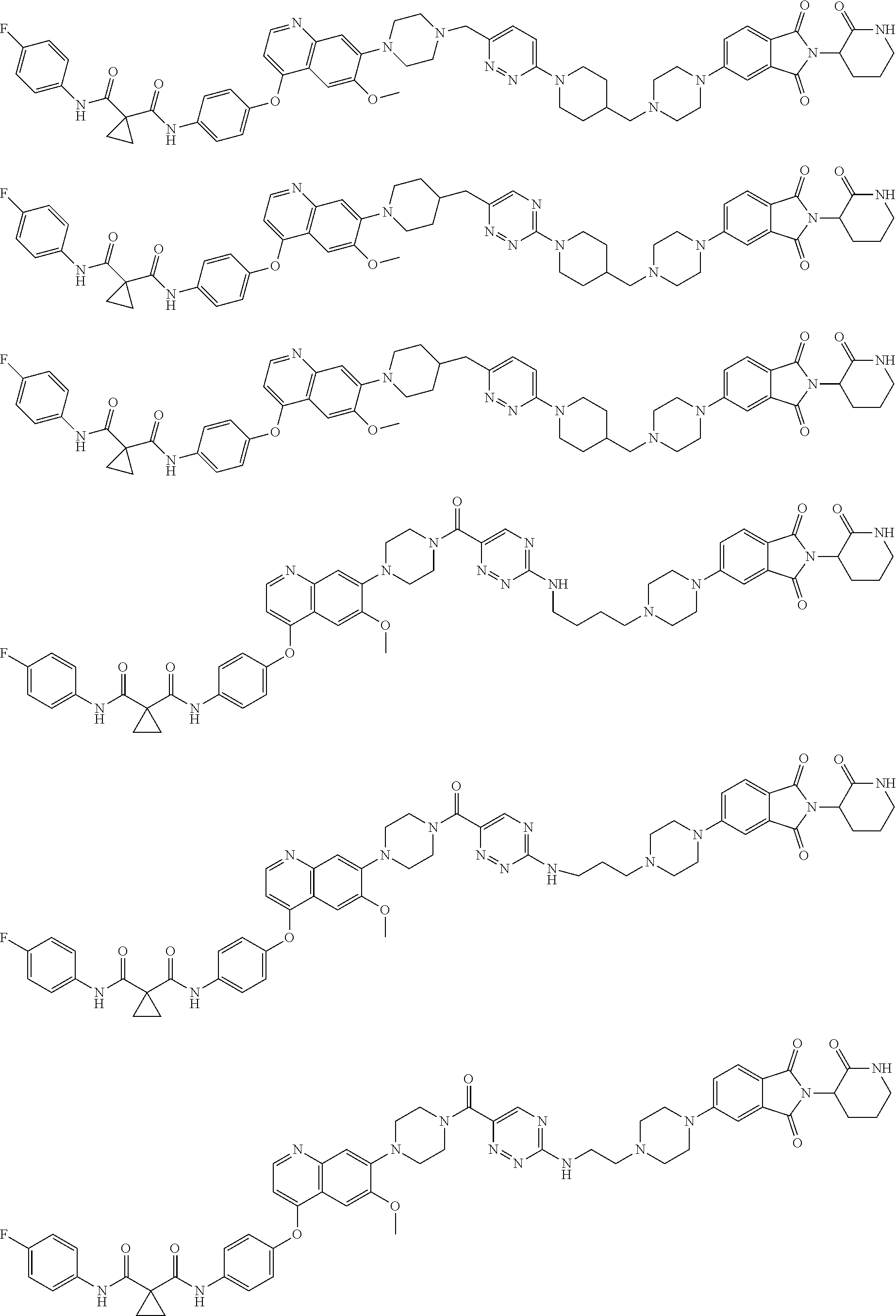

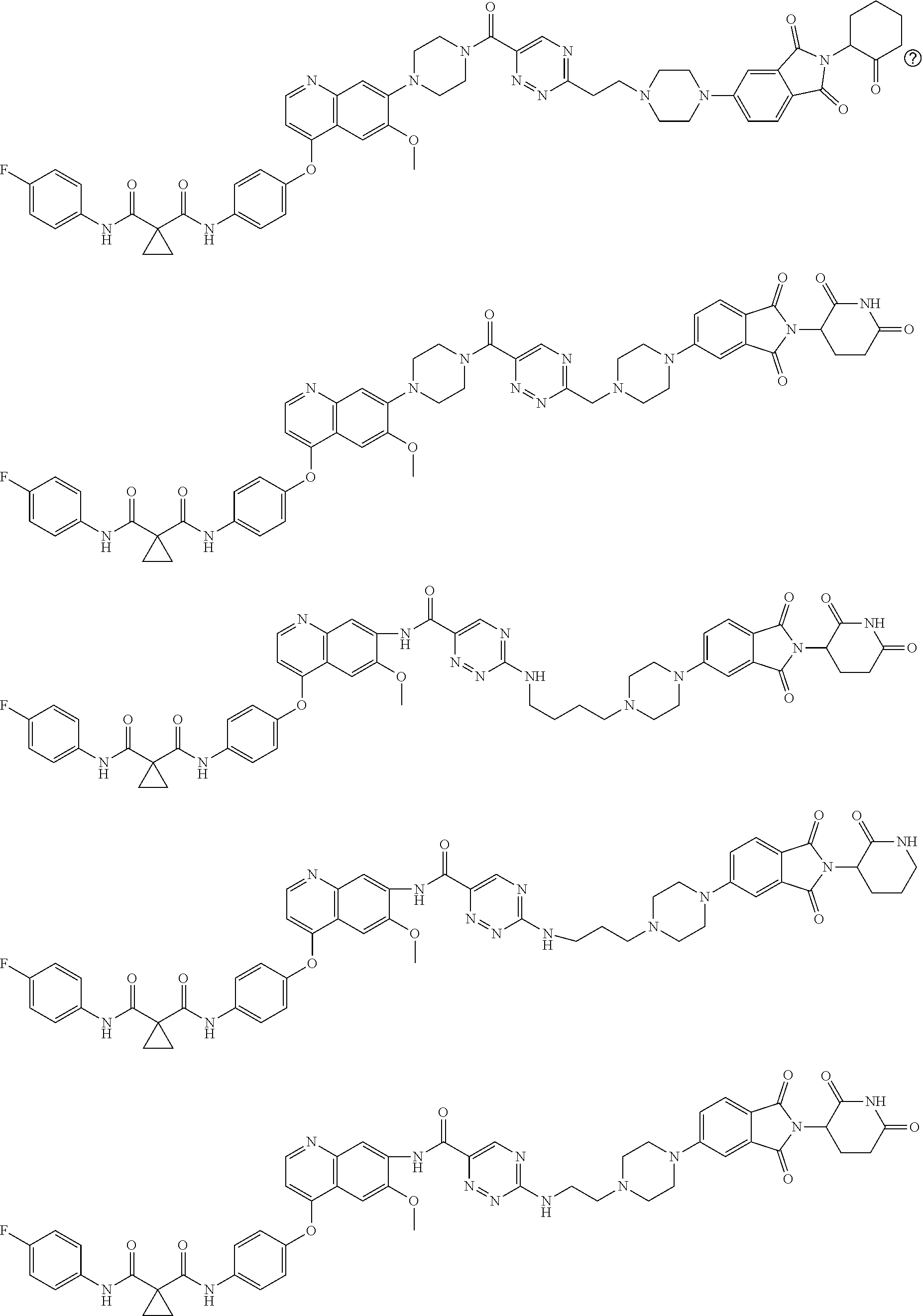

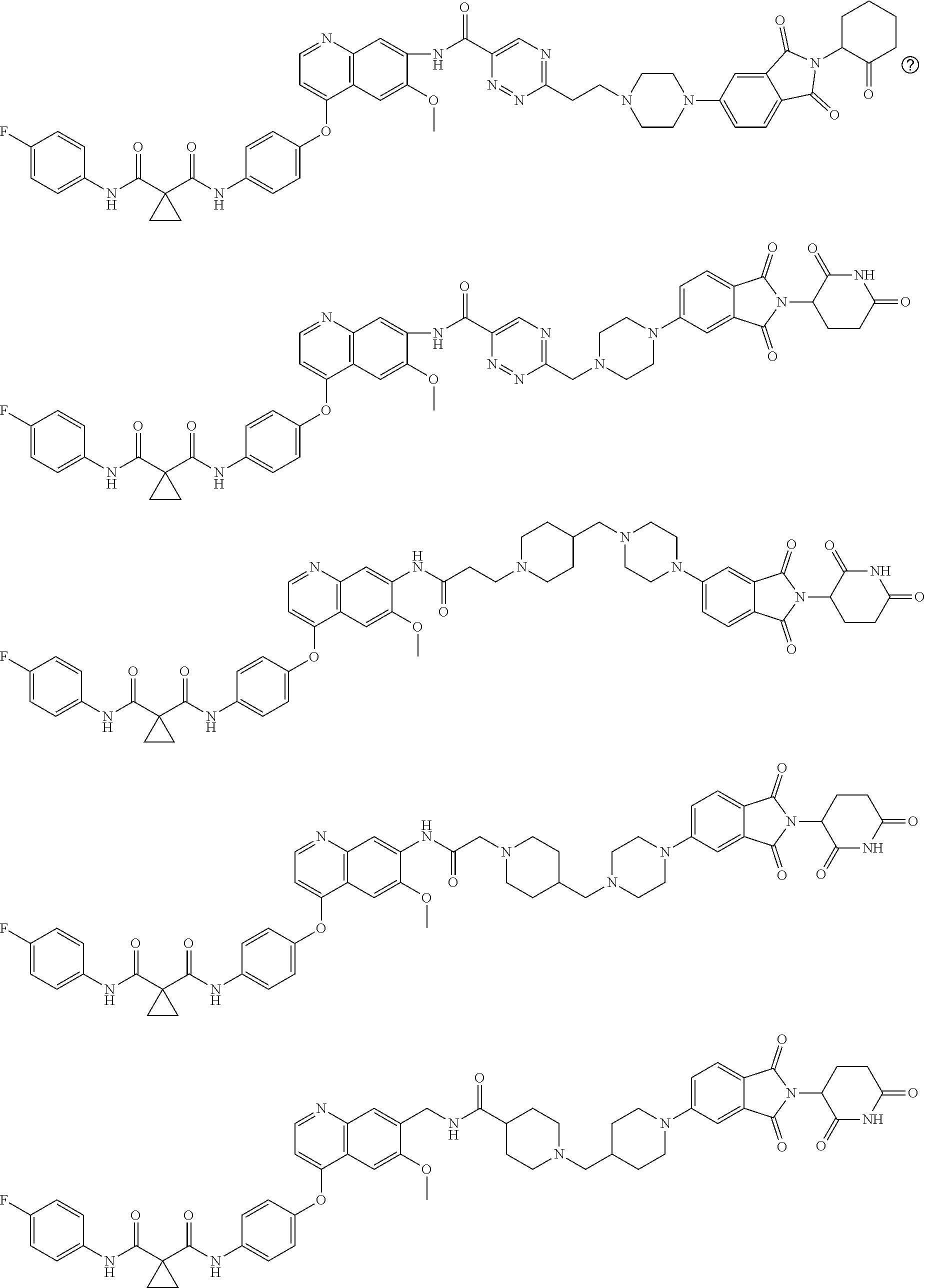

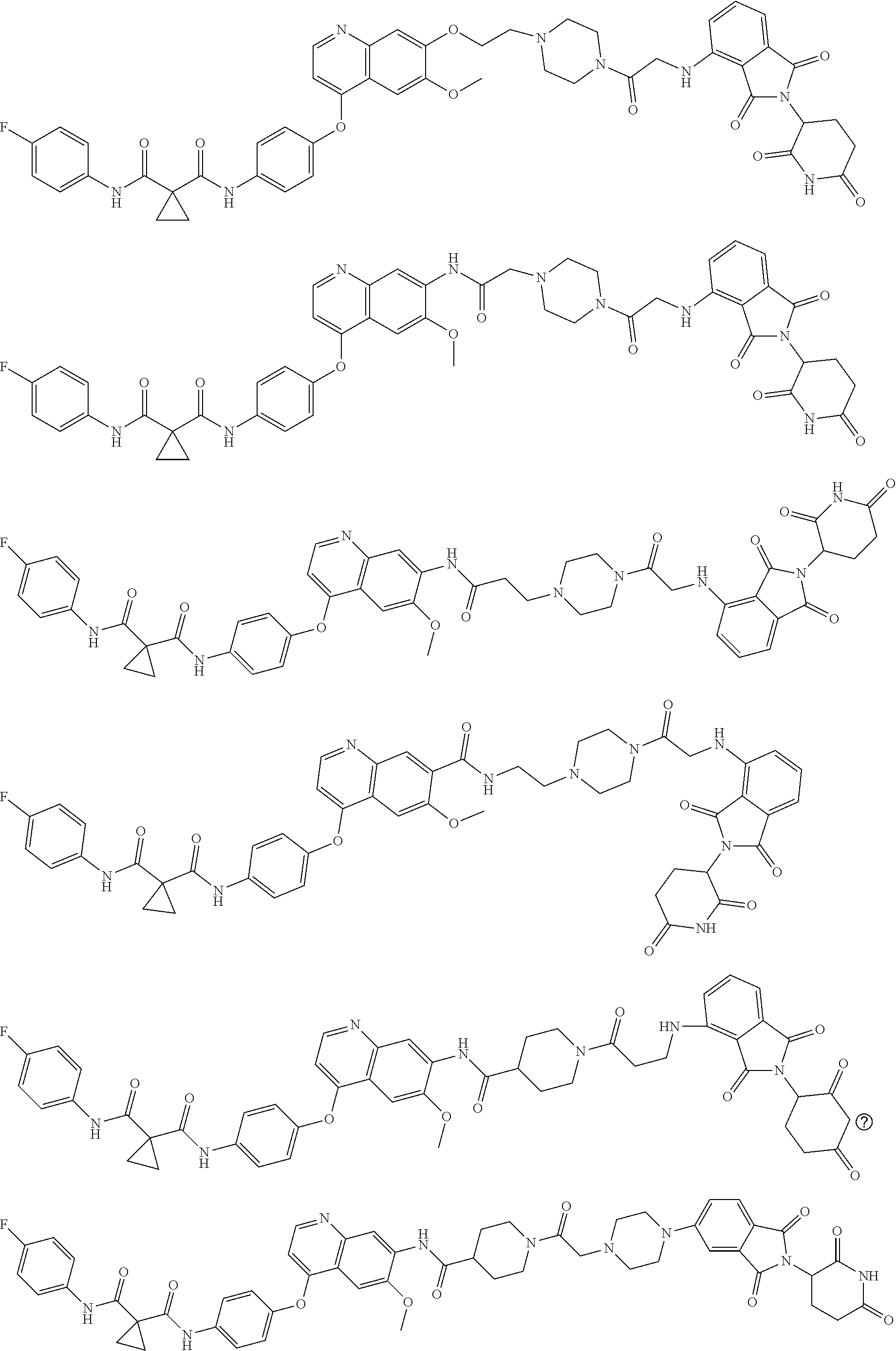

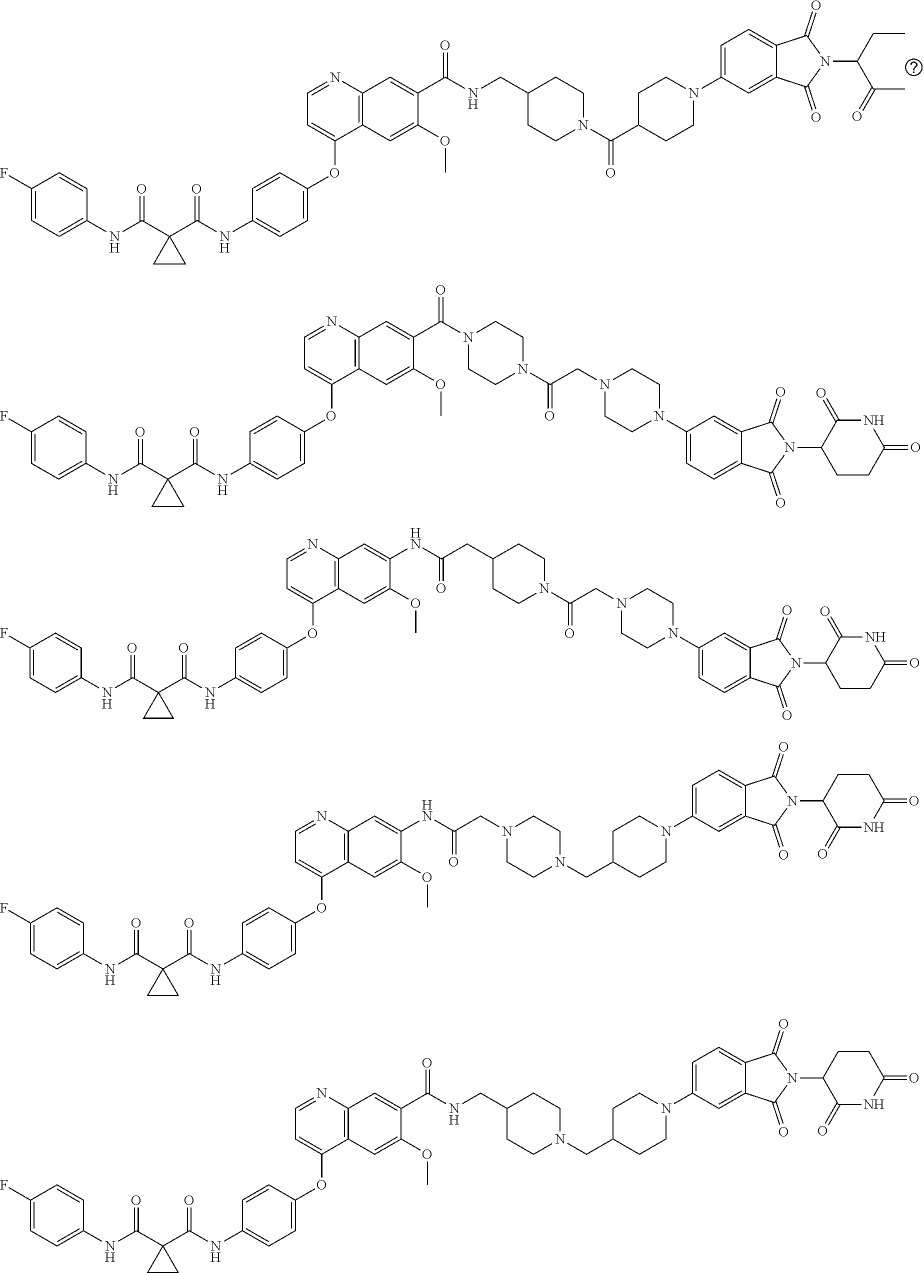

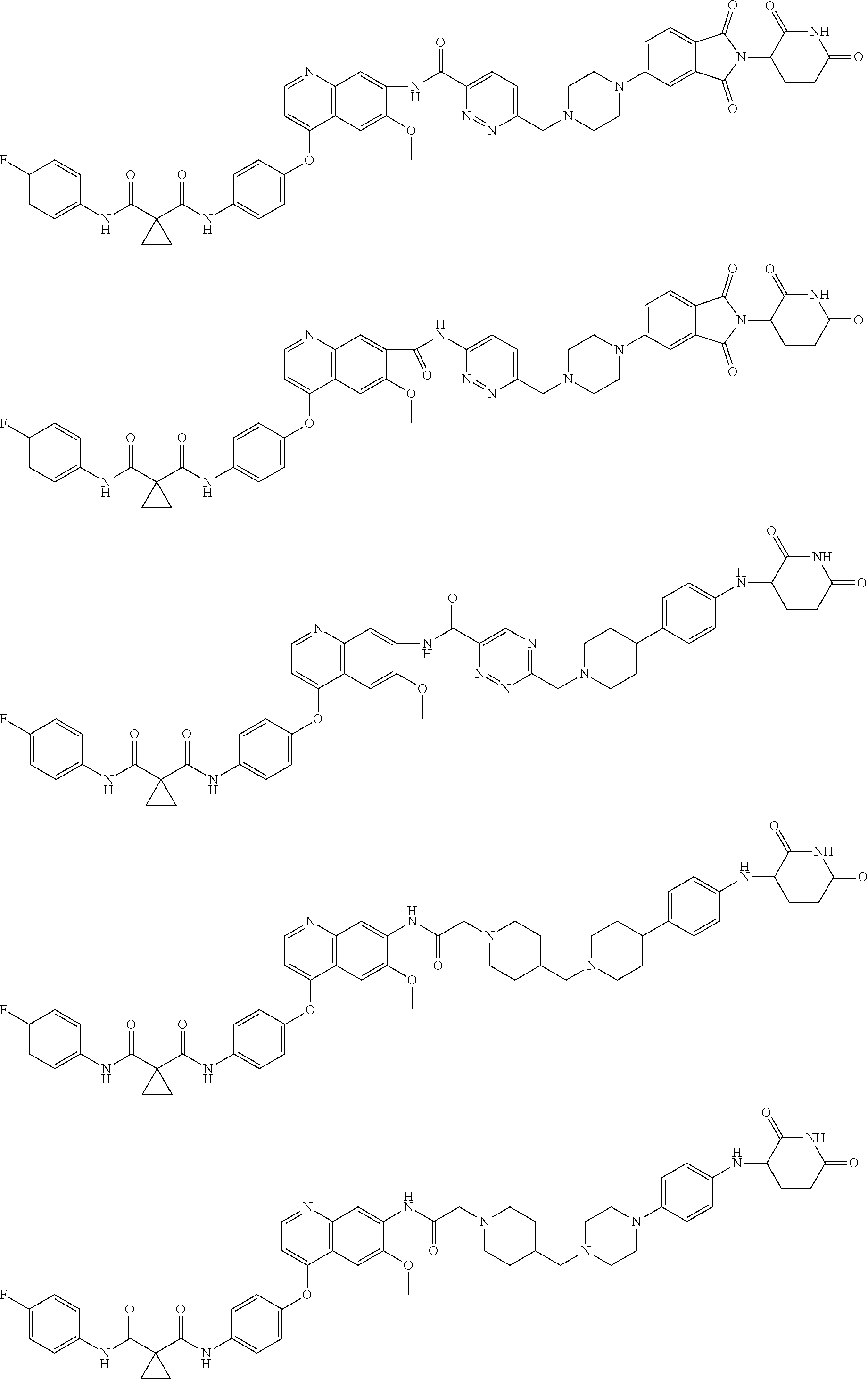

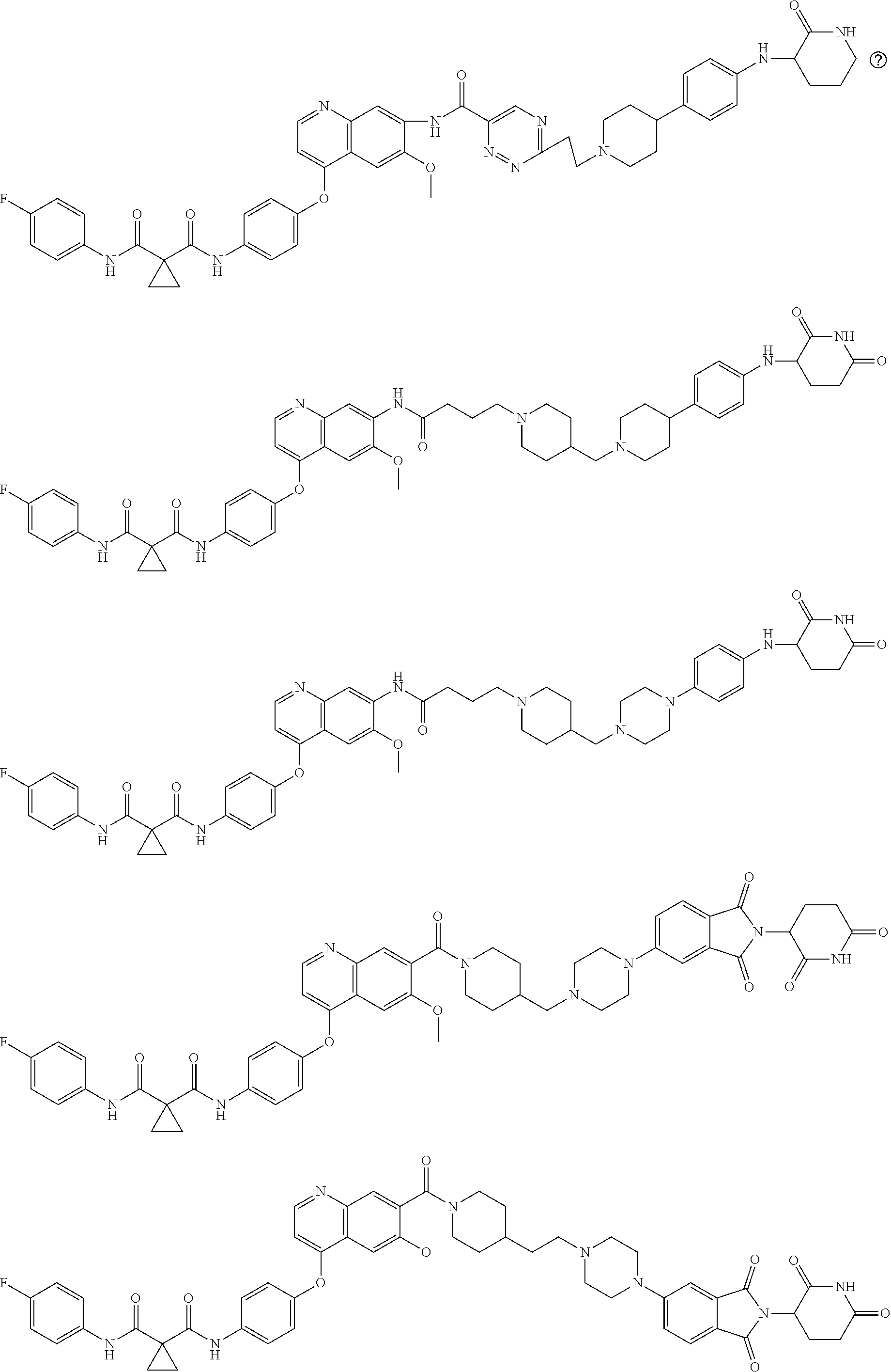

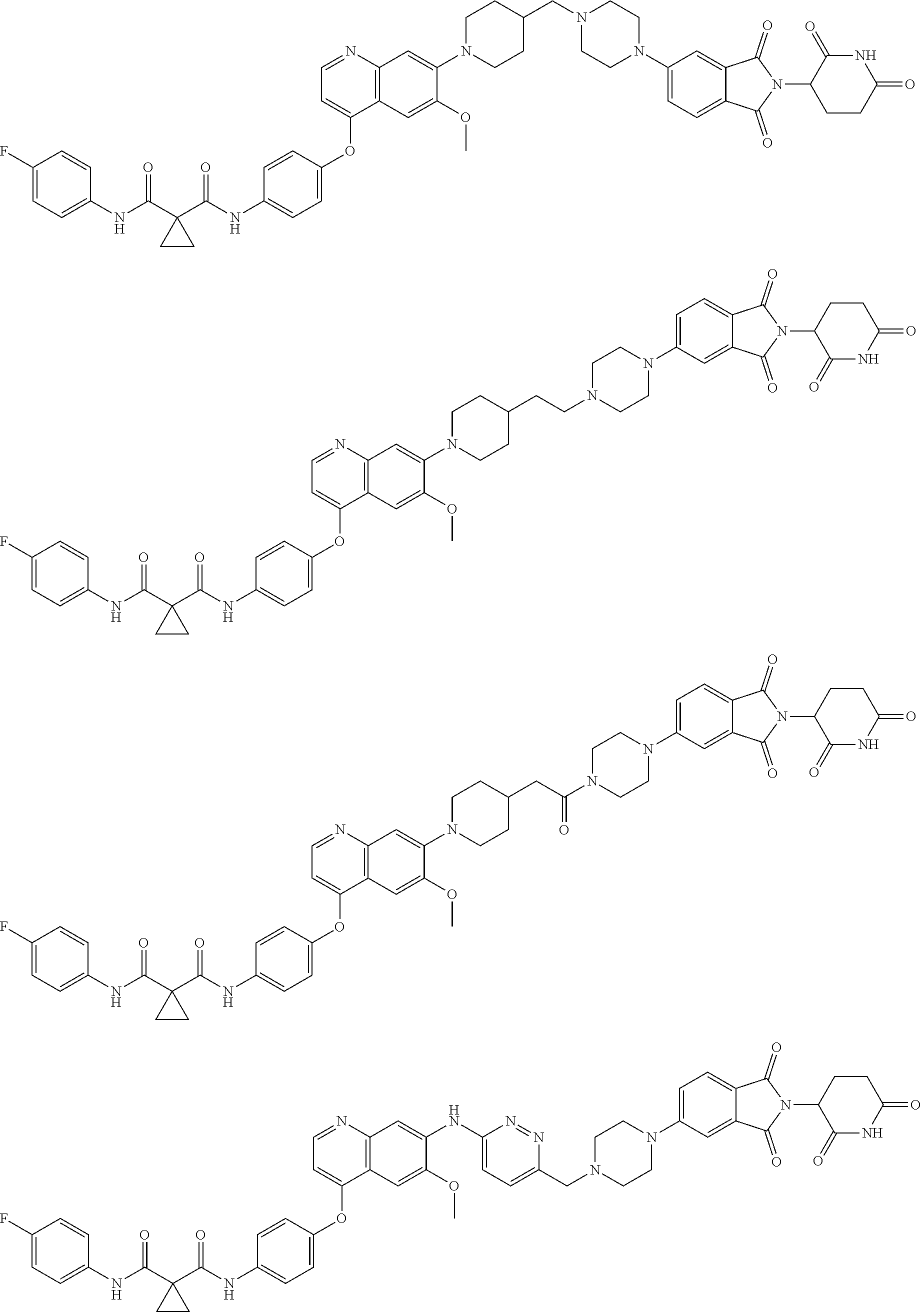 [SMILES: COc1cc2c(Oc3ccc(NC(=O)C4(C(=O)Nc5ccc(F)cc5)CC4)cc3)ccnc2cc1N1CCC(CN2CCN(c3ccc4c(c3)C(=O)N(C3CCC(=O)NC3=O)C4=O)CC2)CC1 ; COc1cc2c(Oc3ccc(NC(=O)C4(C(=O)Nc5ccc(F)cc5)CC4)cc3)ccnc2cc1N1CCC(CCN2CCN(c3ccc4c(c3)C(=O)N(C3CCC(=O)NC3=O)C4=O)CC2)CC1 ; COc1cc2c(Oc3ccc(NC(=O)C4(C(=O)Nc5ccc(F)cc5)CC4)cc3)ccnc2cc1N1CCC(CC(=O)N2CCN(c3ccc4c(c3)C(=O)N(C3CCC(=O)NC3=O)C4=O)CC2)CC1 ; COc1cc2c(Oc3ccc(NC(=O)C4(C(=O)Nc5ccc(F)cc5)CC4)cc3)ccnc2cc1Nc1ccc(CN2CCN(c3ccc4c(c3)C(=O)N(C3CCC(=O)NC3=O)C4=O)CC2)nn1]

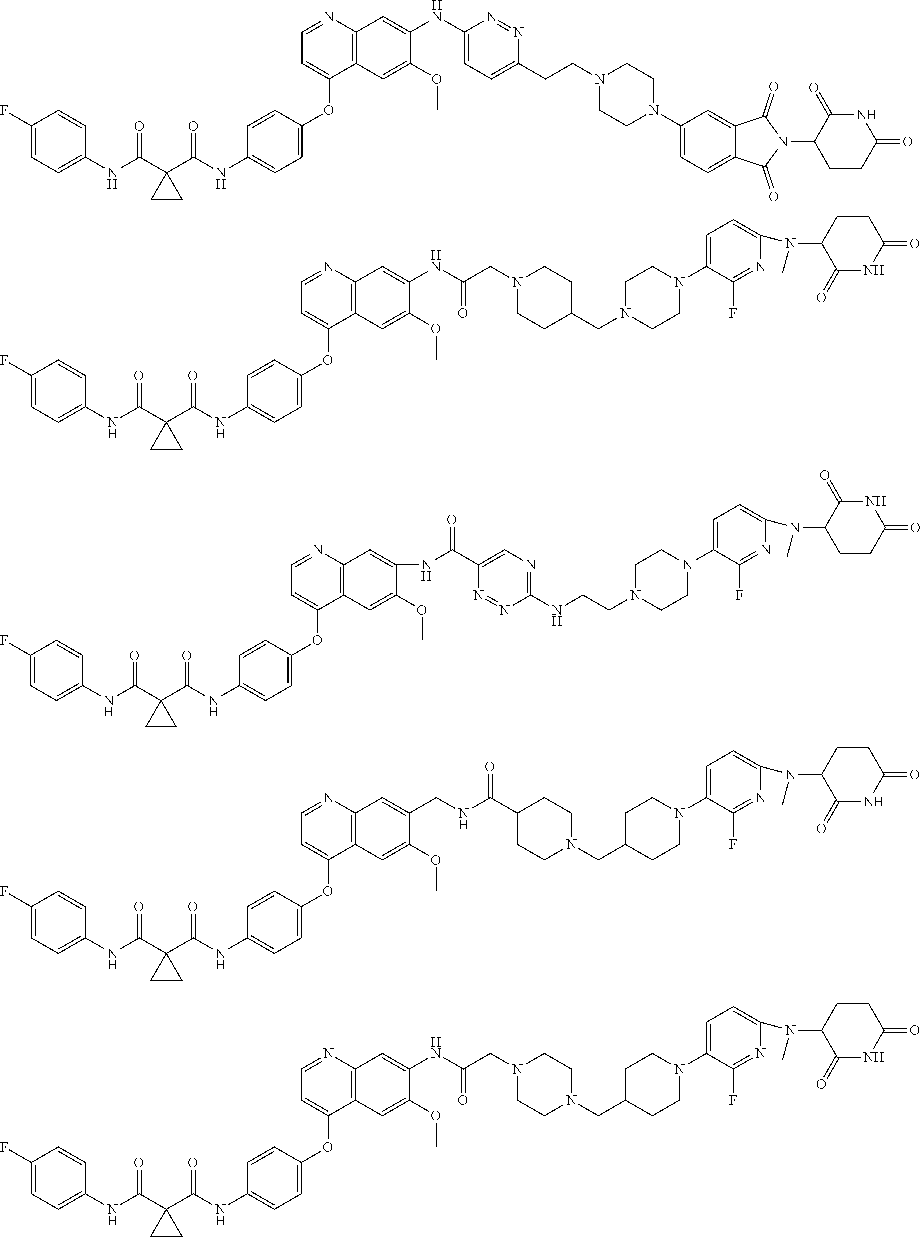

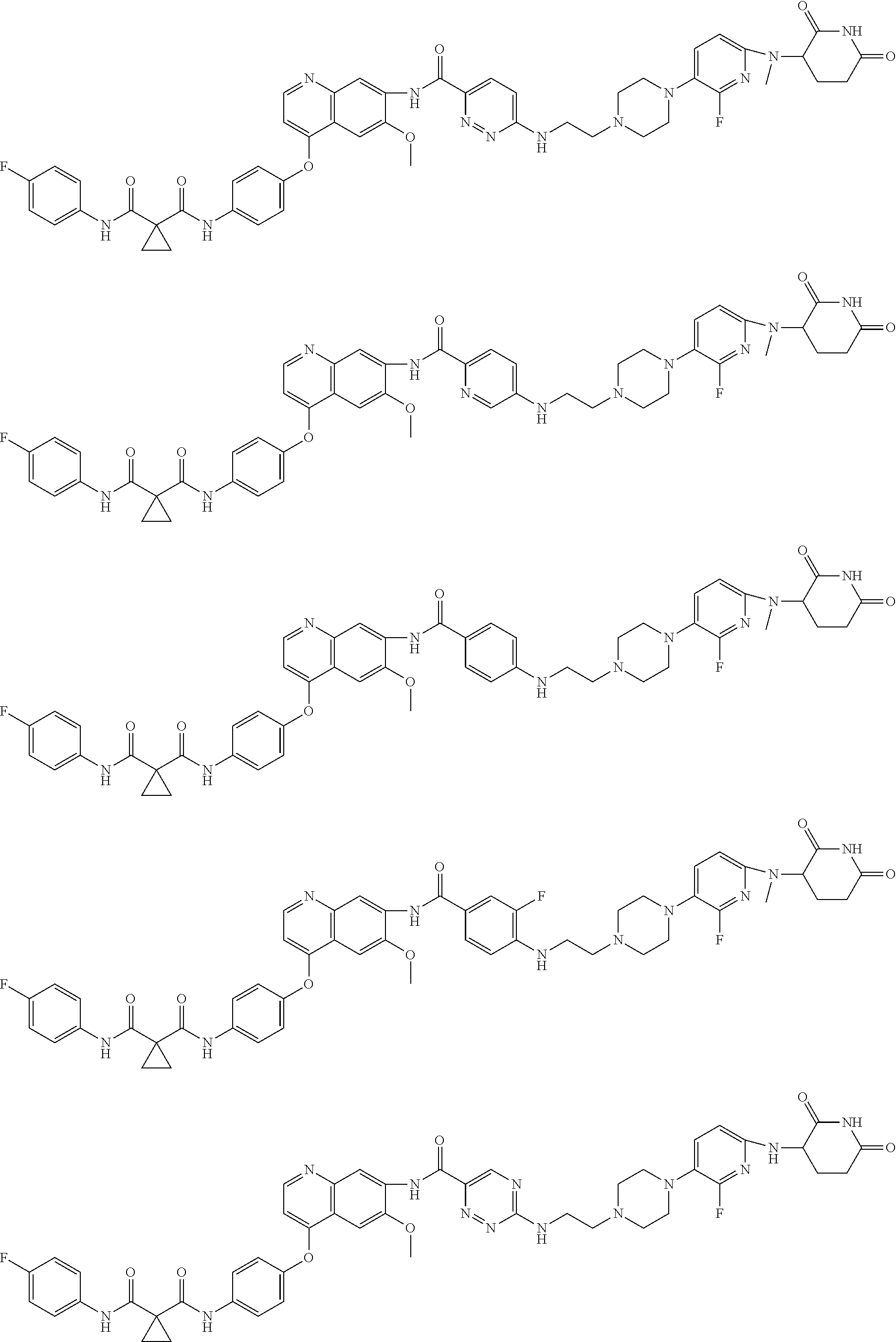

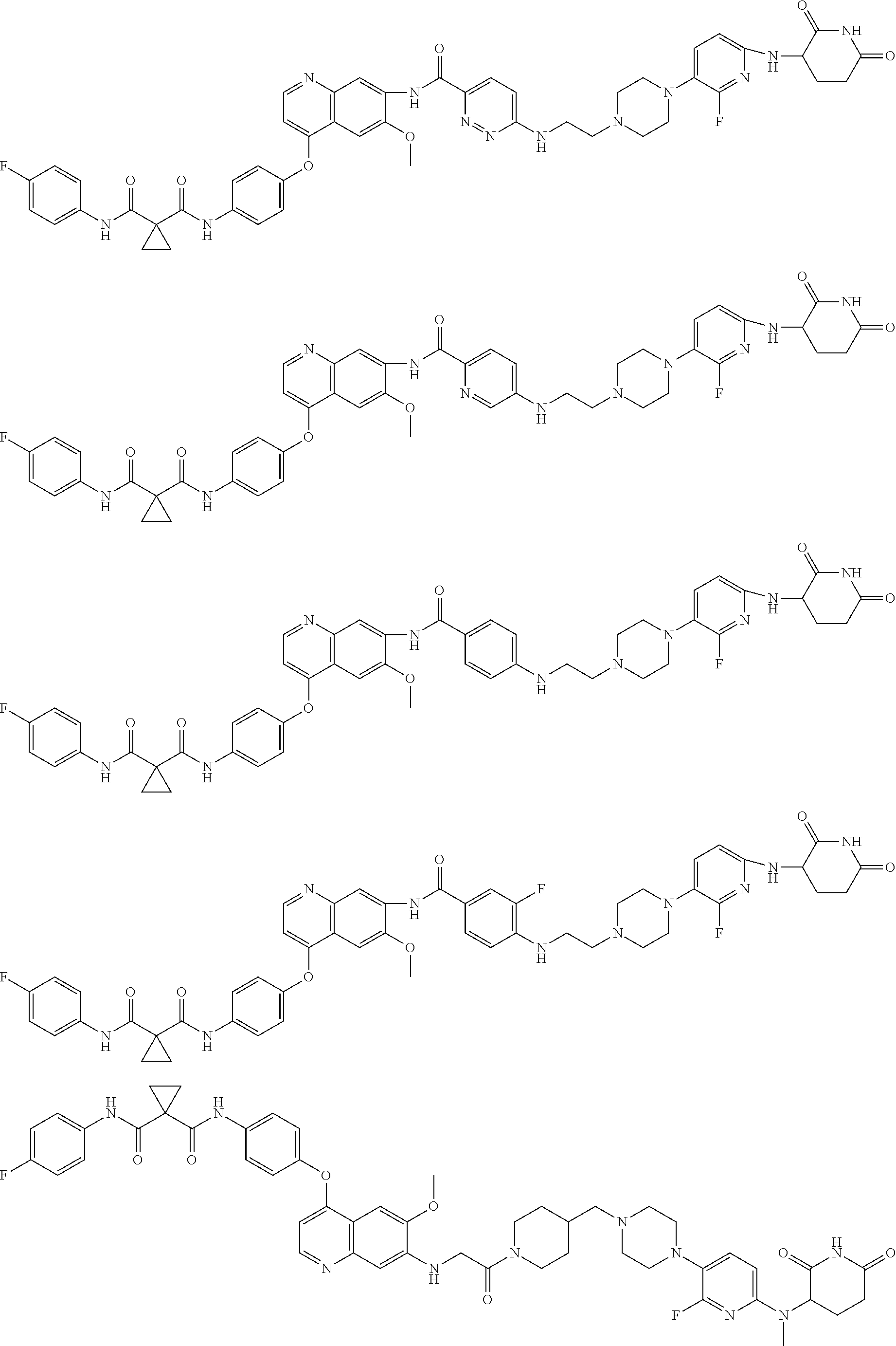

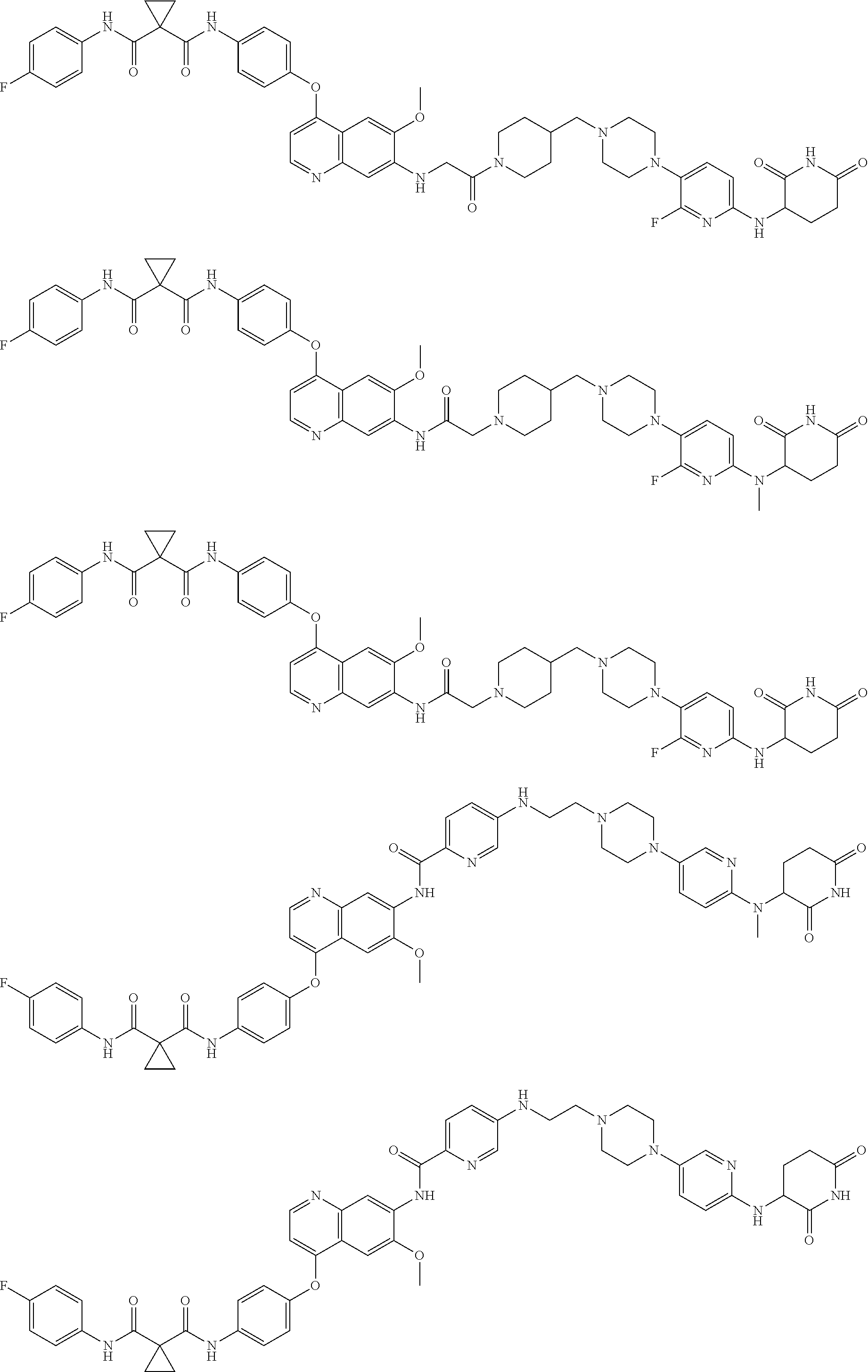

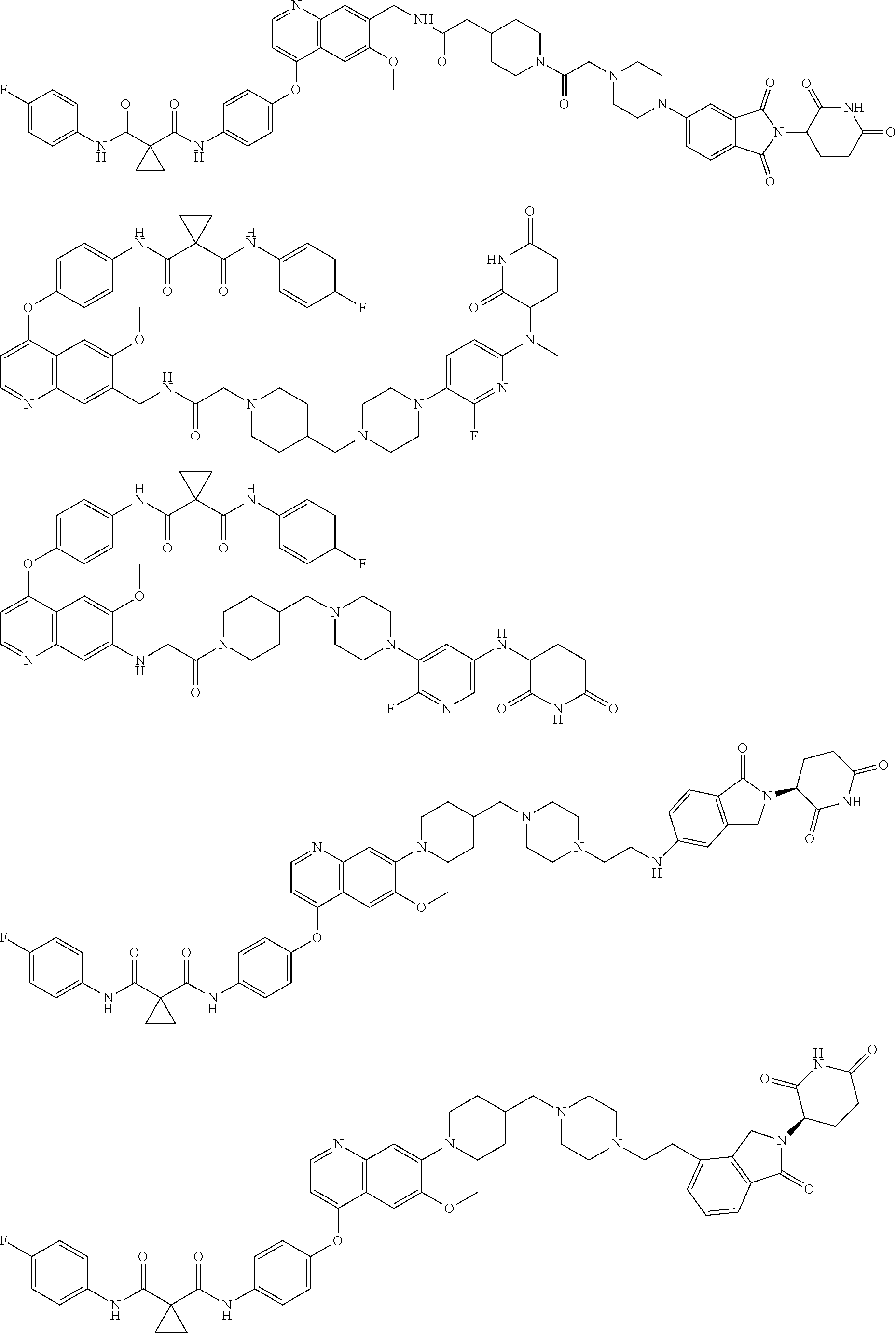

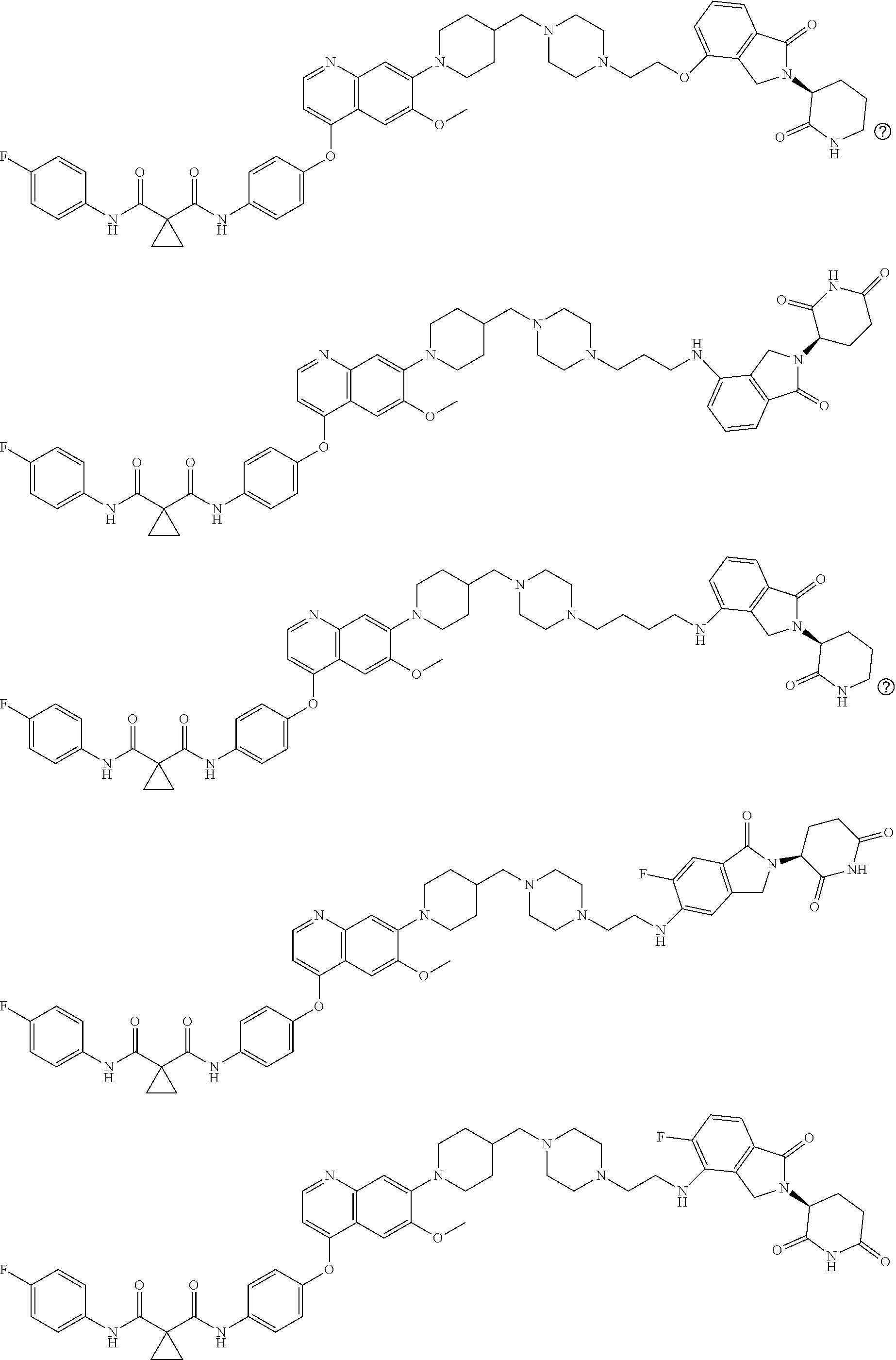

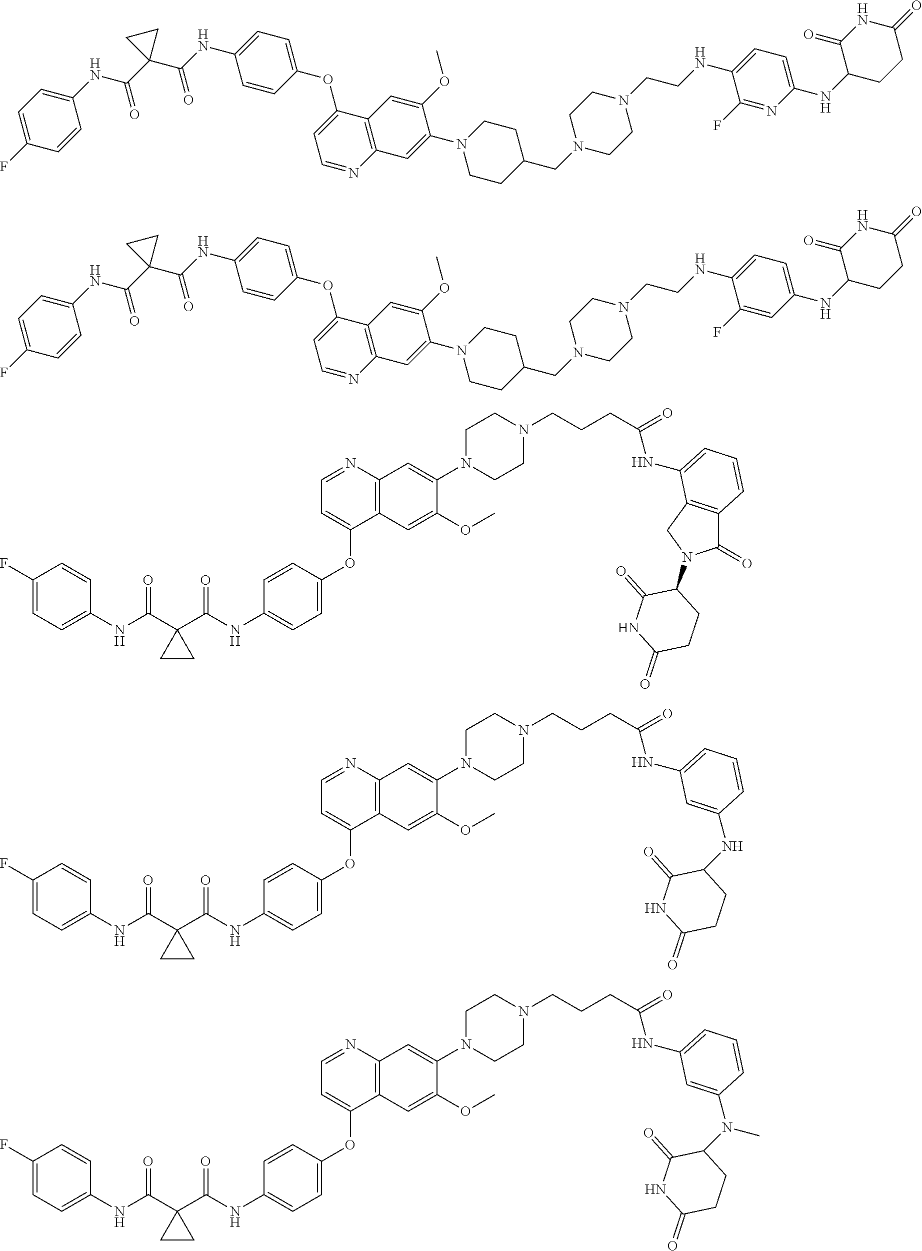

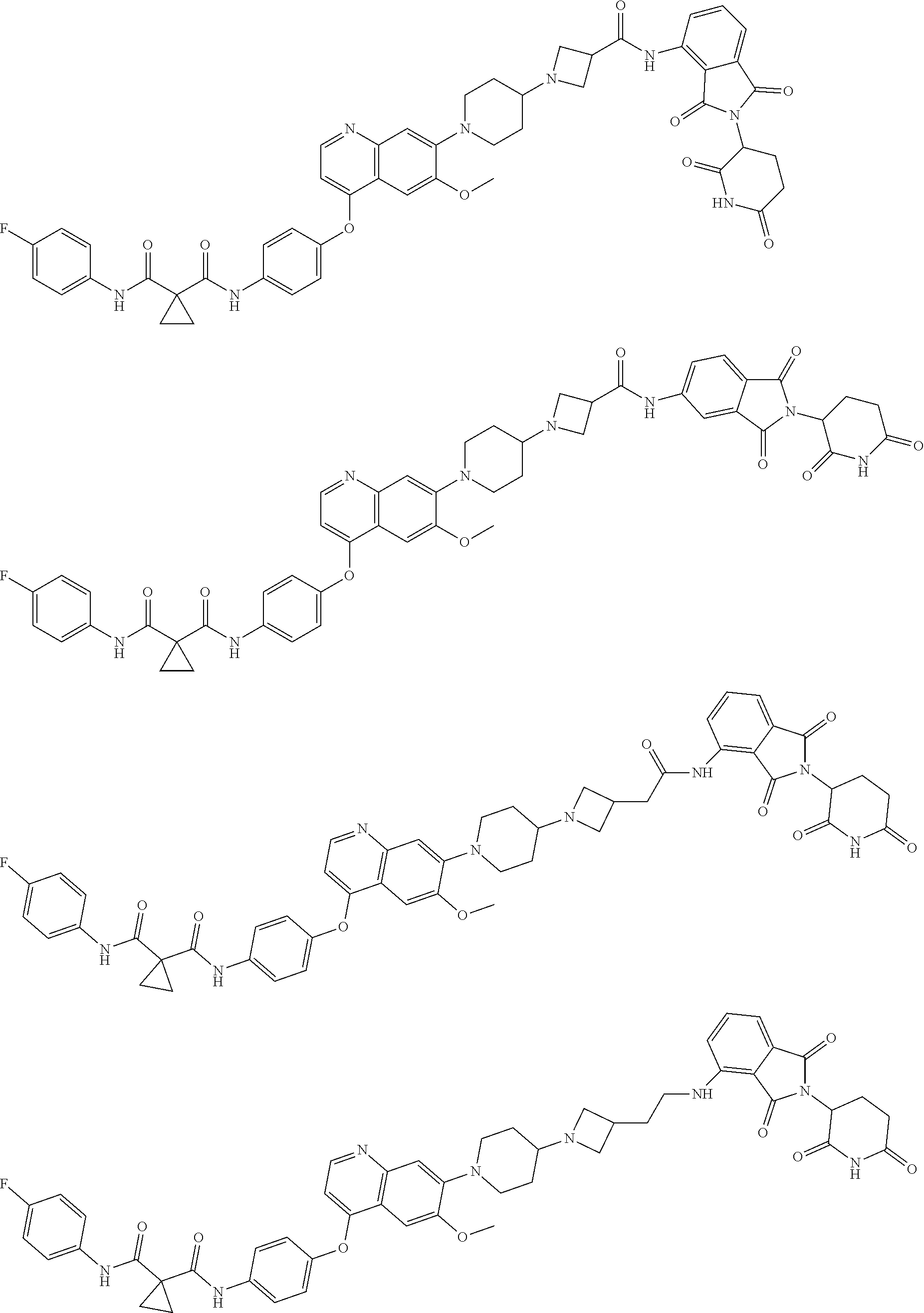 [SMILES: COc1cc2c(Oc3ccc(NC(=O)C4(C(=O)Nc5ccc(F)cc5)CC4)cc3)ccnc2cc1N1CCC(N2CC(C(=O)Nc3cccc4c3C(=O)N(C3CCC(=O)NC3=O)C4=O)C2)CC1 ; COc1cc2c(Oc3ccc(NC(=O)C4(C(=O)Nc5ccc(F)cc5)CC4)cc3)ccnc2cc1N1CCC(N2CC(C(=O)Nc3ccc4c(c3)C(=O)N(C3CCC(=O)NC3=O)C4=O)C2)CC1 ; COc1cc2c(Oc3ccc(NC(=O)C4(C(=O)Nc5ccc(F)cc5)CC4)cc3)ccnc2cc1N1CCC(N2CC(CC(=O)Nc3cccc4c3C(=O)N(C3CCC(=O)NC3=O)C4=O)C2)CC1 ; COc1cc2c(Oc3ccc(NC(=O)C4(C(=O)Nc5ccc(F)cc5)CC4)cc3)ccnc2cc1N1CCC(N2CC(CCNc3cccc4c3C(=O)N(C3CCC(=O)NC3=O)C4=O)C2)CC1]

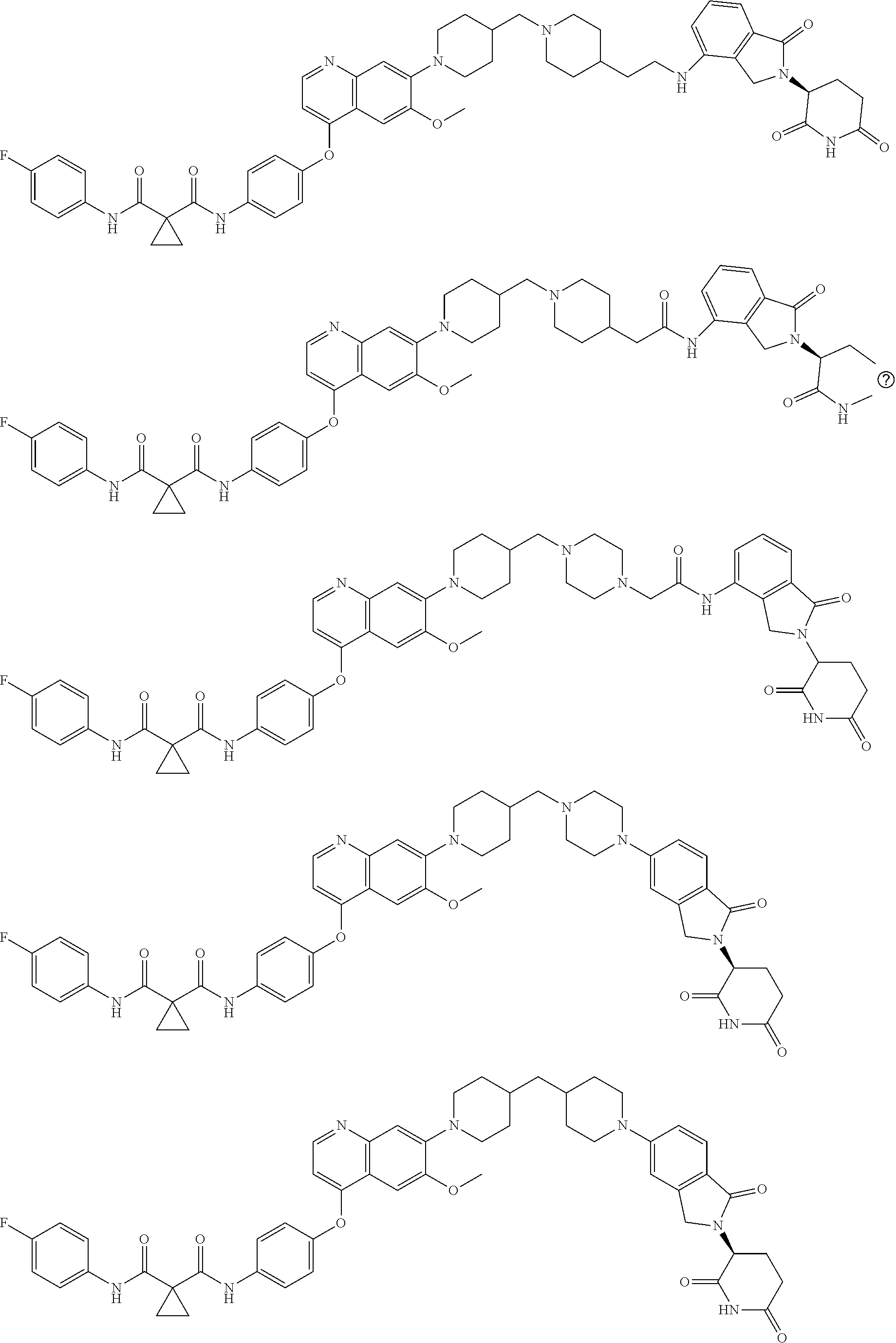

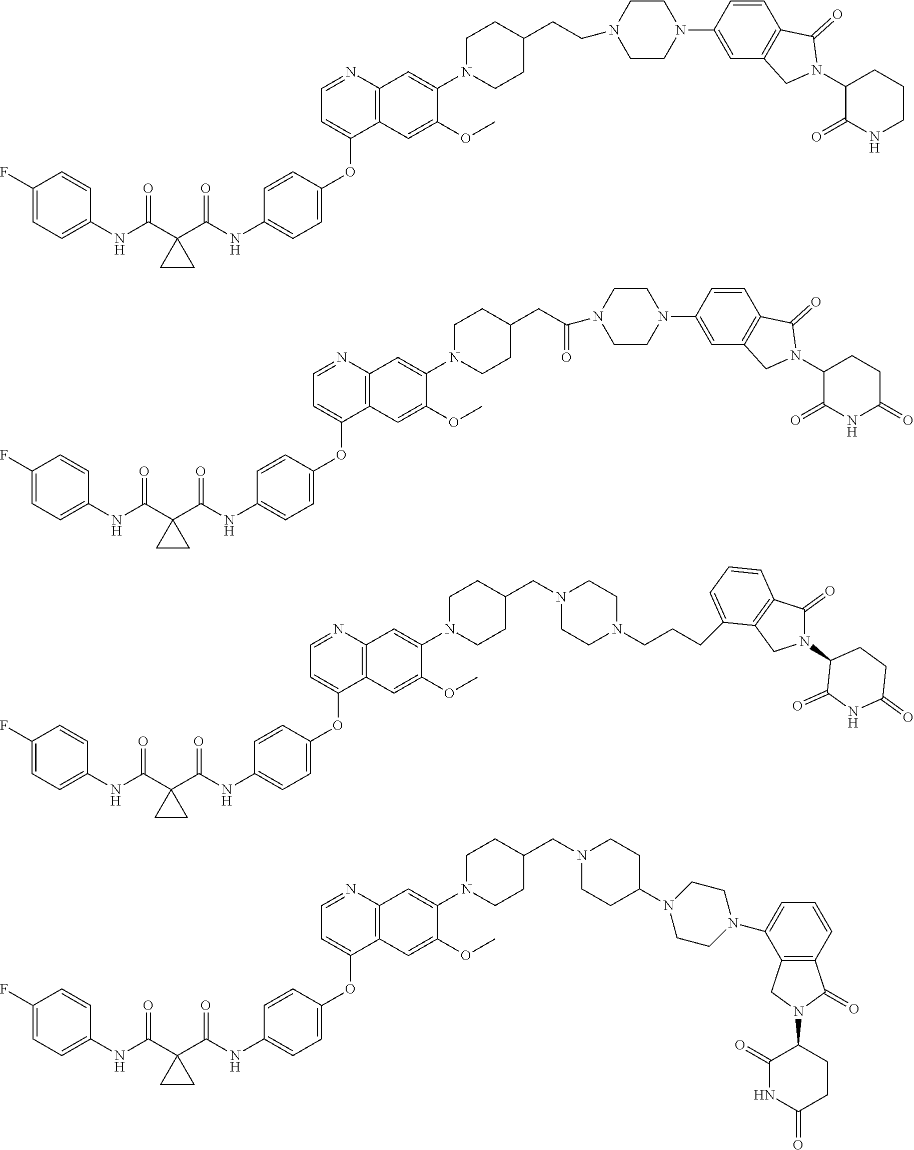 [SMILES: COc1cc2c(Oc3ccc(NC(=O)C4(C(=O)Nc5ccc(F)cc5)CC4)cc3)ccnc2cc1N1CCC(CCN2CCN(c3ccc4c(c3)CN(C3CCCNC3=O)C4=O)CC2)CC1 ; COc1cc2c(Oc3ccc(NC(=O)C4(C(=O)Nc5ccc(F)cc5)CC4)cc3)ccnc2cc1N1CCC(CC(=O)N2CCN(c3ccc4c(c3)CN(C3CCC(=O)NC3=O)C4=O)CC2)CC1 ; COc1cc2c(Oc3ccc(NC(=O)C4(C(=O)Nc5ccc(F)cc5)CC4)cc3)ccnc2cc1N1CCC(CN2CCN(CCCc3cccc4c3CN([C@H]3CCC(=O)NC3=O)C4=O)CC2)CC1 ; COc1cc2c(Oc3ccc(NC(=O)C4(C(=O)Nc5ccc(F)cc5)CC4)cc3)ccnc2cc1N1CCC(CN2CCC(N3CCN(c4cccc5c4CN([C@H]4CCC(=O)NC4=O)C5=O)CC3)CC2)CC1]

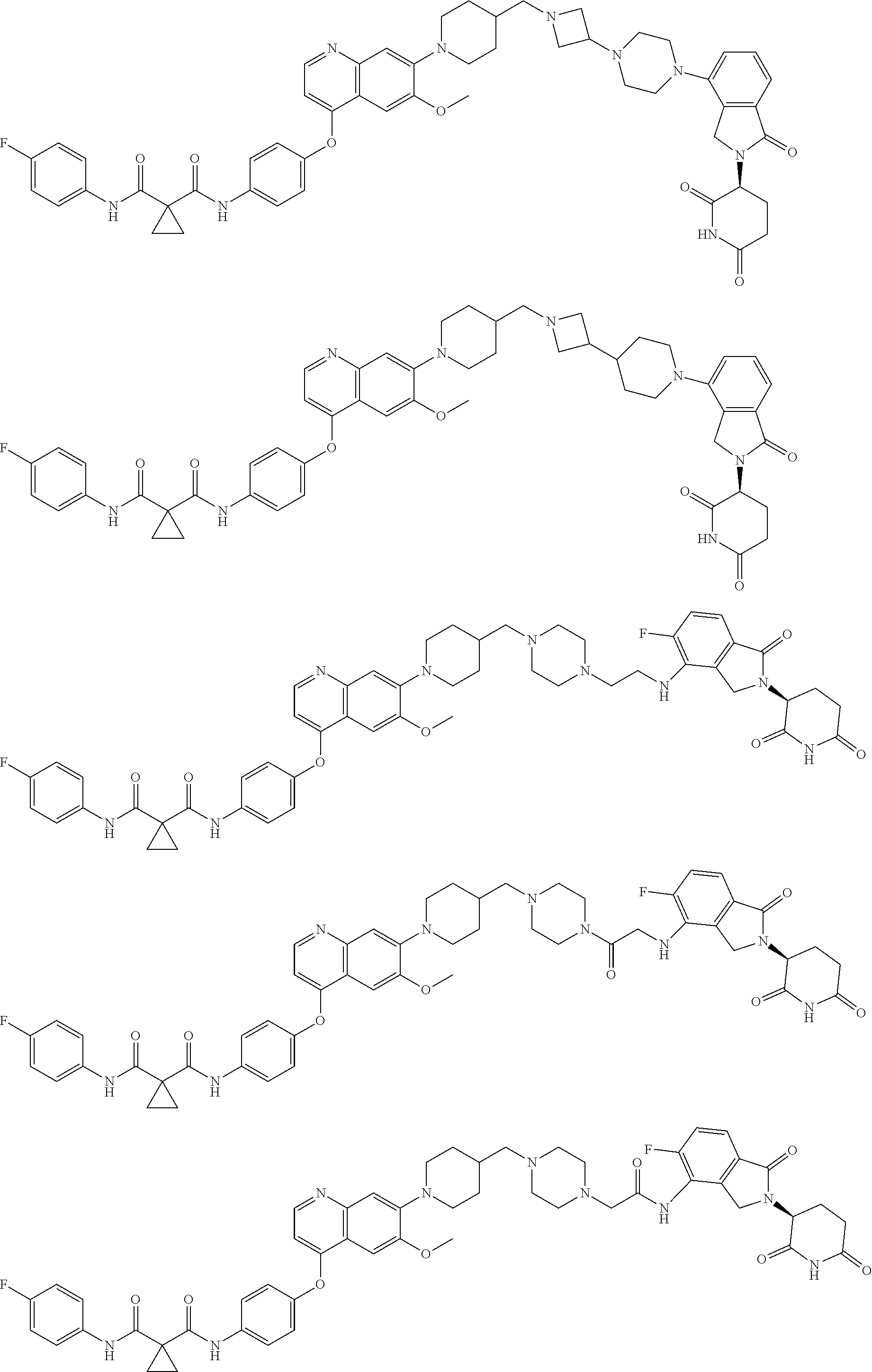

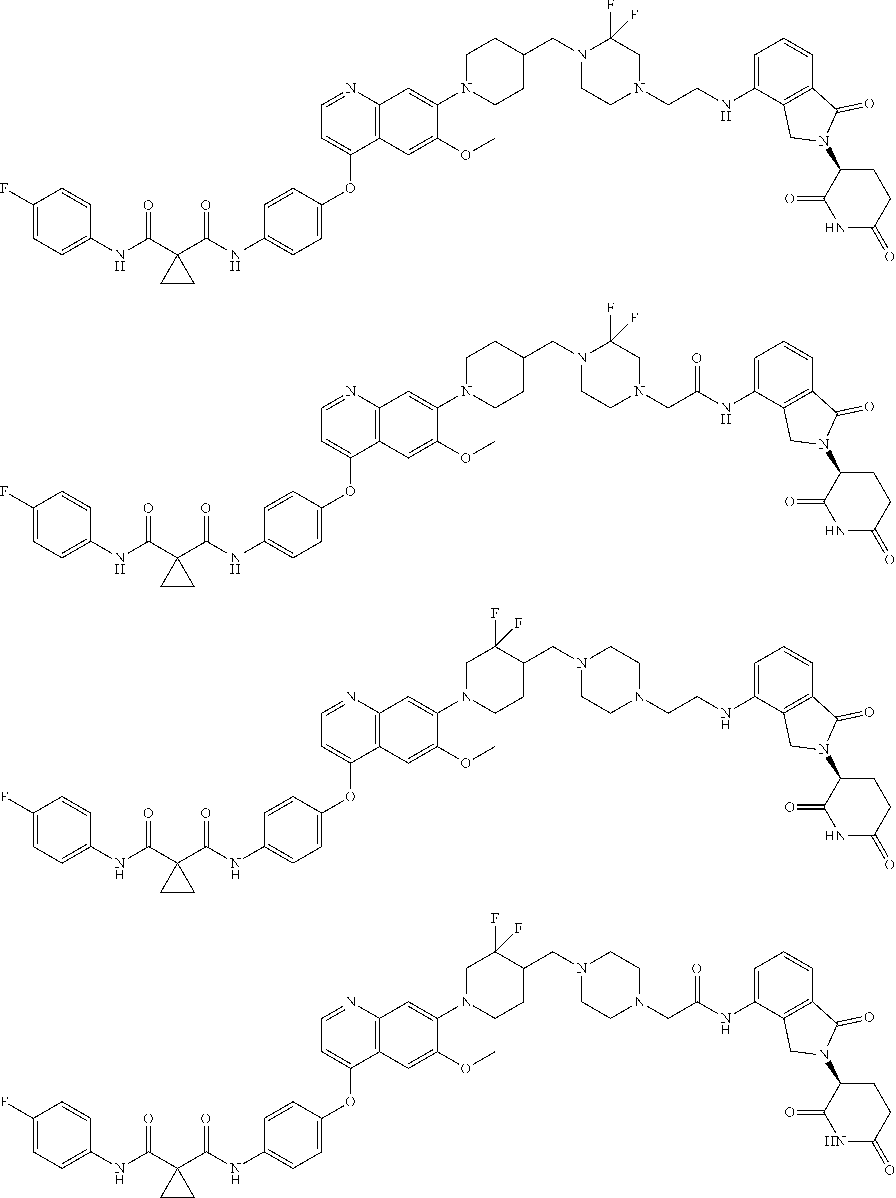

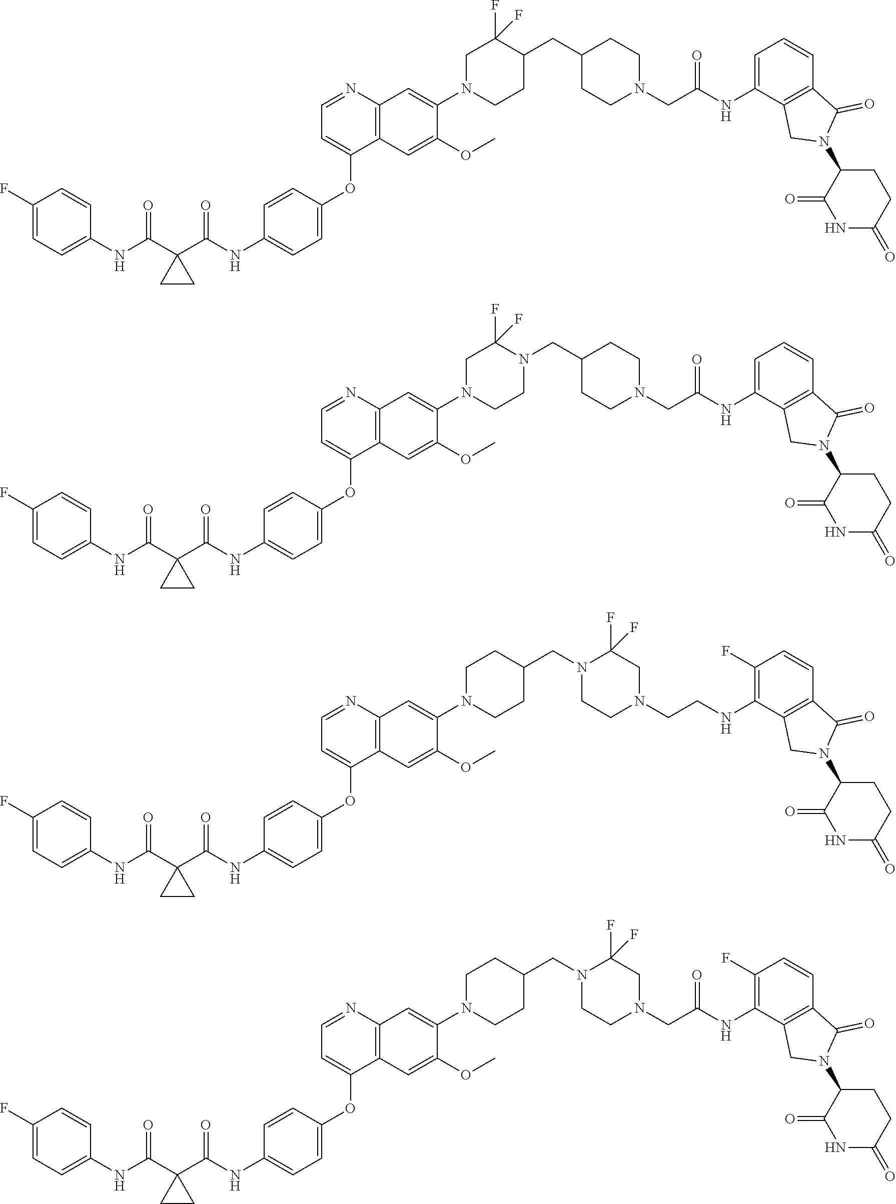

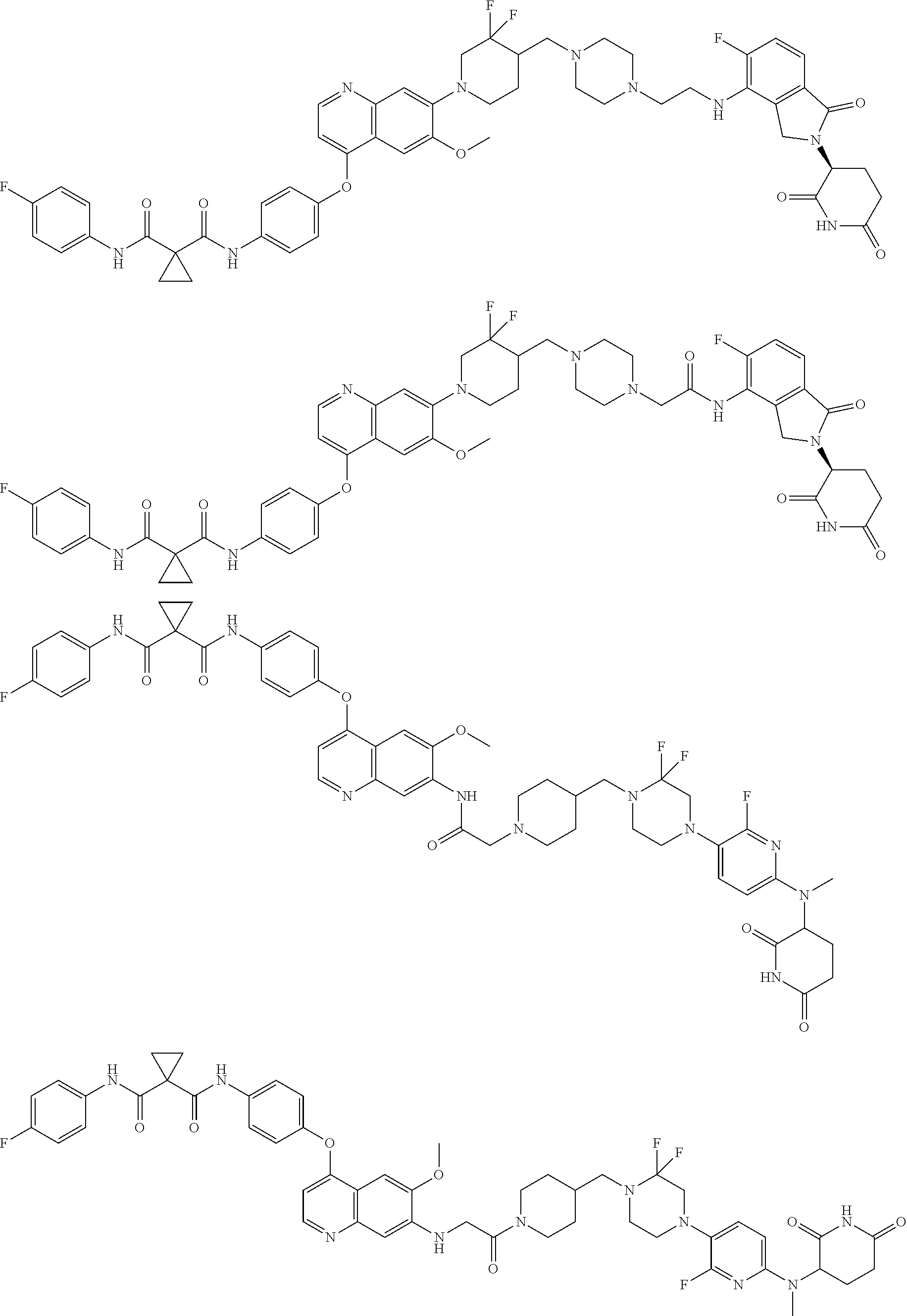

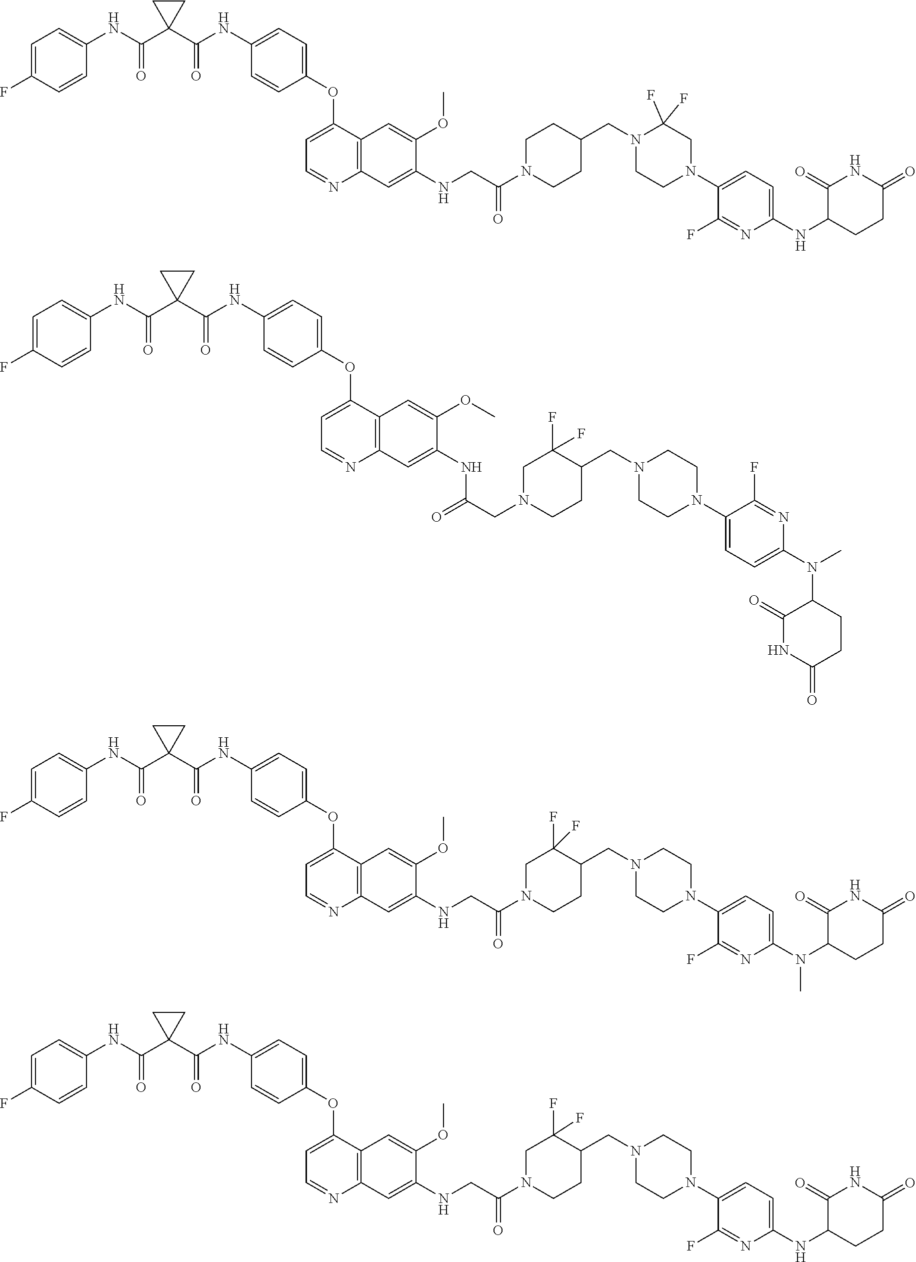 [SMILES: COc1cc2c(Oc3ccc(NC(=O)C4(C(=O)Nc5ccc(F)cc5)CC4)cc3)ccnc2cc1NCC(=O)N1CCC(CN2CCN(c3ccc(NC4CCC(=O)NC4=O)nc3F)CC2(F)F)CC1 ; COc1cc2c(Oc3ccc(NC(=O)C4(C(=O)Nc5ccc(F)cc5)CC4)cc3)ccnc2cc1NC(=O)CN1CCC(CN2CCN(c3ccc(N(C)C4CCC(=O)NC4=O)nc3F)CC2)C(F)(F)C1 ; COc1cc2c(Oc3ccc(NC(=O)C4(C(=O)Nc5ccc(F)cc5)CC4)cc3)ccnc2cc1NCC(=O)N1CCC(CN2CCN(c3ccc(N(C)C4CCC(=O)NC4=O)nc3F)CC2)C(F)(F)C1 ; COc1cc2c(Oc3ccc(NC(=O)C4(C(=O)Nc5ccc(F)cc5)CC4)cc3)ccnc2cc1NCC(=O)N1CCC(CN2CCN(c3ccc(NC4CCC(=O)NC4=O)nc3F)CC2)C(F)(F)C1]

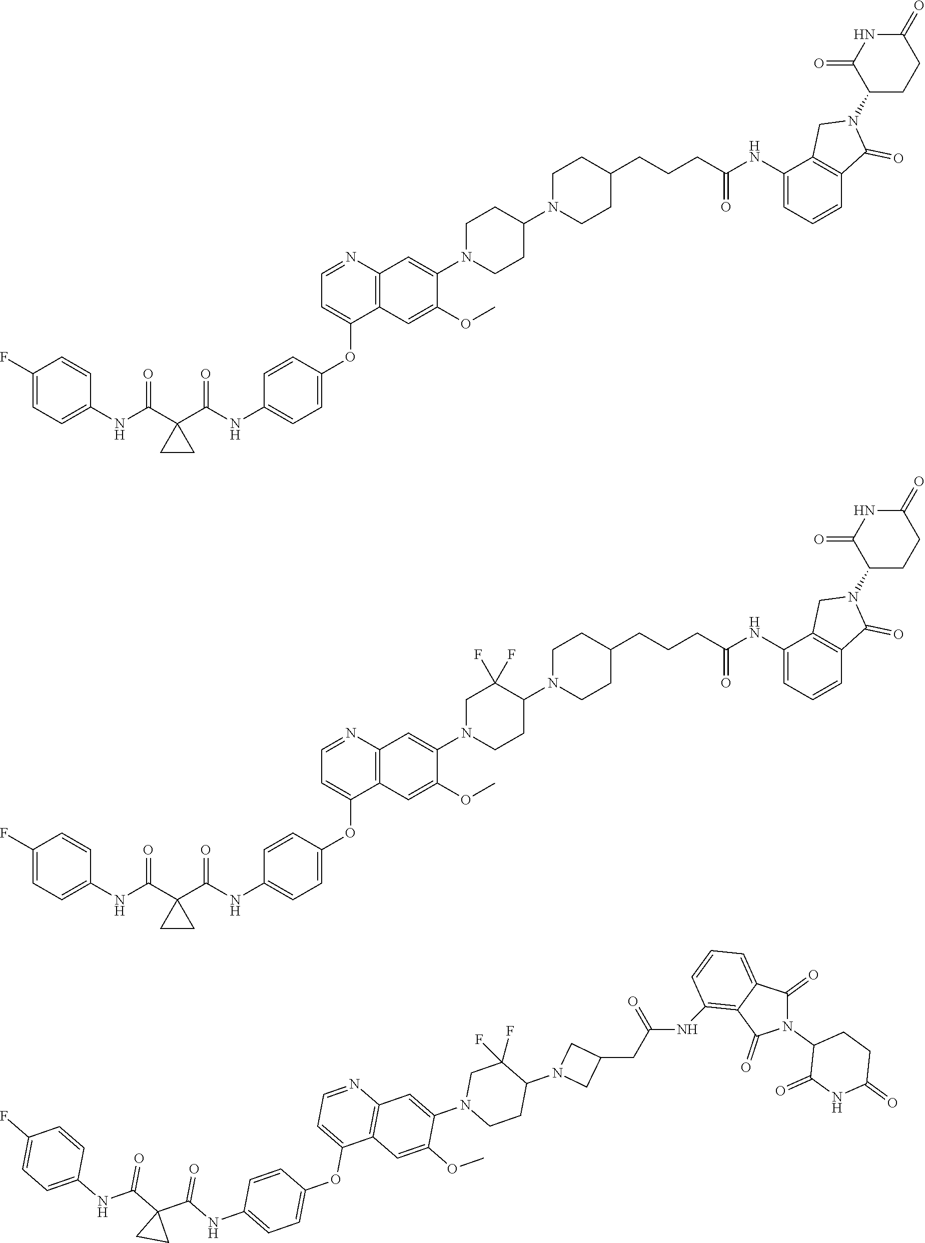 [SMILES: COc1cc2c(Oc3ccc(NC(=O)C4(C(=O)Nc5ccc(F)cc5)CC4)cc3)ccnc2cc1N1CCC(N2CCC(CCCC(=O)Nc3cccc4c3CN([C@H]3CCC(=O)NC3=O)C4=O)CC2)CC1 ; COc1cc2c(Oc3ccc(NC(=O)C4(C(=O)Nc5ccc(F)cc5)CC4)cc3)ccnc2cc1N1CCC(N2CCC(CCCC(=O)Nc3cccc4c3CN([C@H]3CCC(=O)NC3=O)C4=O)CC2)C(F)(F)C1 ; COc1cc2c(Oc3ccc(NC(=O)C4(C(=O)Nc5ccc(F)cc5)CC4)cc3)ccnc2cc1N1CCC(N2CC(CC(=O)Nc3cccc4c3C(=O)N(C3CCC(=O)NC3=O)C4=O)C2)C(F)(F)C1]

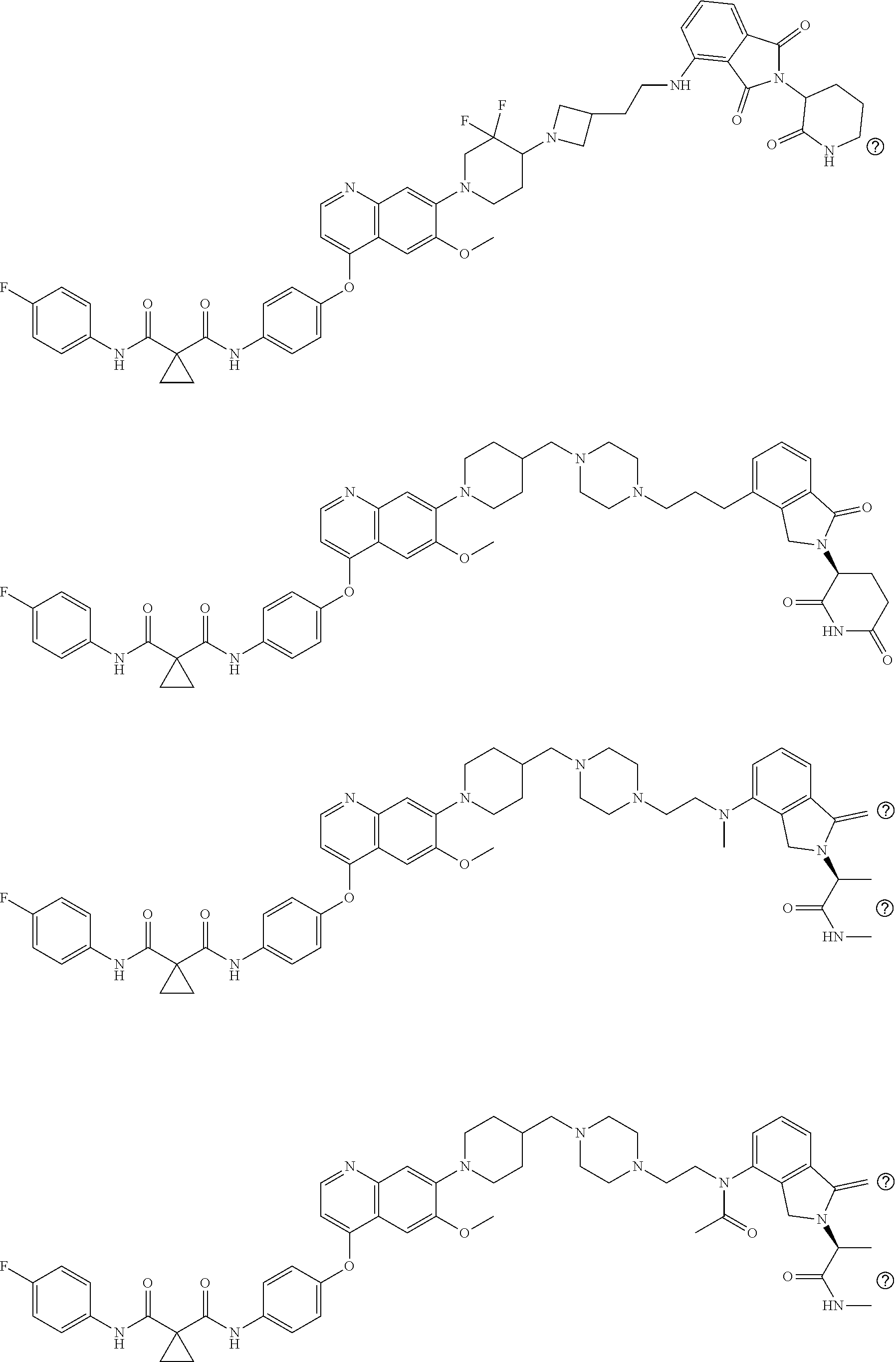 [SMILES: COc1cc2c(Oc3ccc(NC(=O)C4(C(=O)Nc5ccc(F)cc5)CC4)cc3)ccnc2cc1N1CCC(N2CC(CCNc3cccc4c3C(=O)N(C3CCCNC3=O)C4=O)C2)C(F)(F)C1 ; COc1cc2c(Oc3ccc(NC(=O)C4(C(=O)Nc5ccc(F)cc5)CC4)cc3)ccnc2cc1N1CCC(CN2CCN(CCCc3cccc4c3CN([C@H]3CCC(=O)NC3=O)C4=O)CC2)CC1 ; C=C1c2cccc(N(C)CCN3CCN(CC4CCN(c5cc6nccc(Oc7ccc(NC(=O)C8(C(=O)Nc9ccc(F)cc9)CC8)cc7)c6cc5OC)CC4)CC3)c2CN1[C@@H](C)C(=O)NC ; C=C1c2cccc(N(CCN3CCN(CC4CCN(c5cc6nccc(Oc7ccc(NC(=O)C8(C(=O)Nc9ccc(F)cc9)CC8)cc7)c6cc5OC)CC4)CC3)C(C)=O)c2CN1[C@@H](C)C(=O)NC]

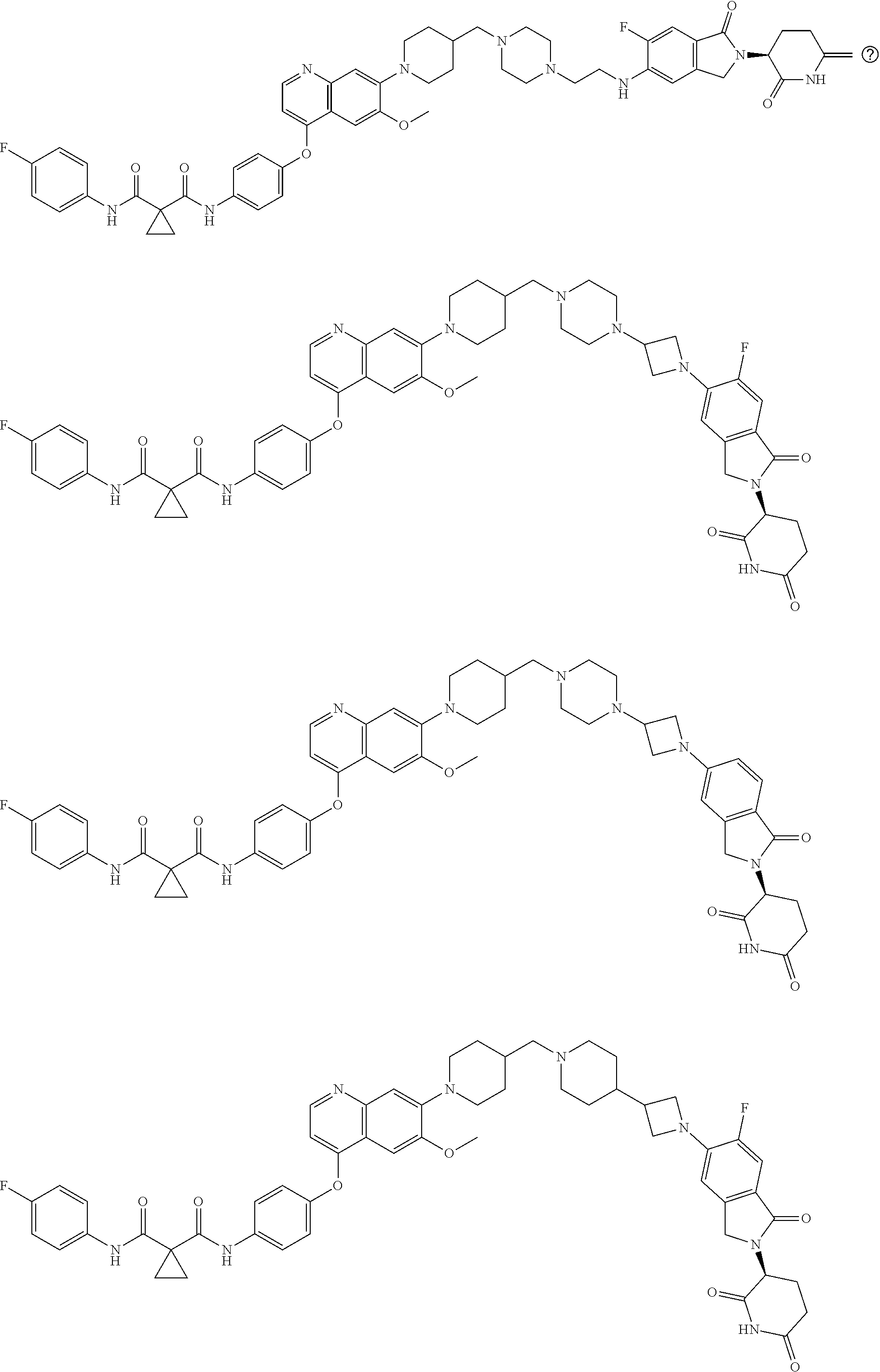

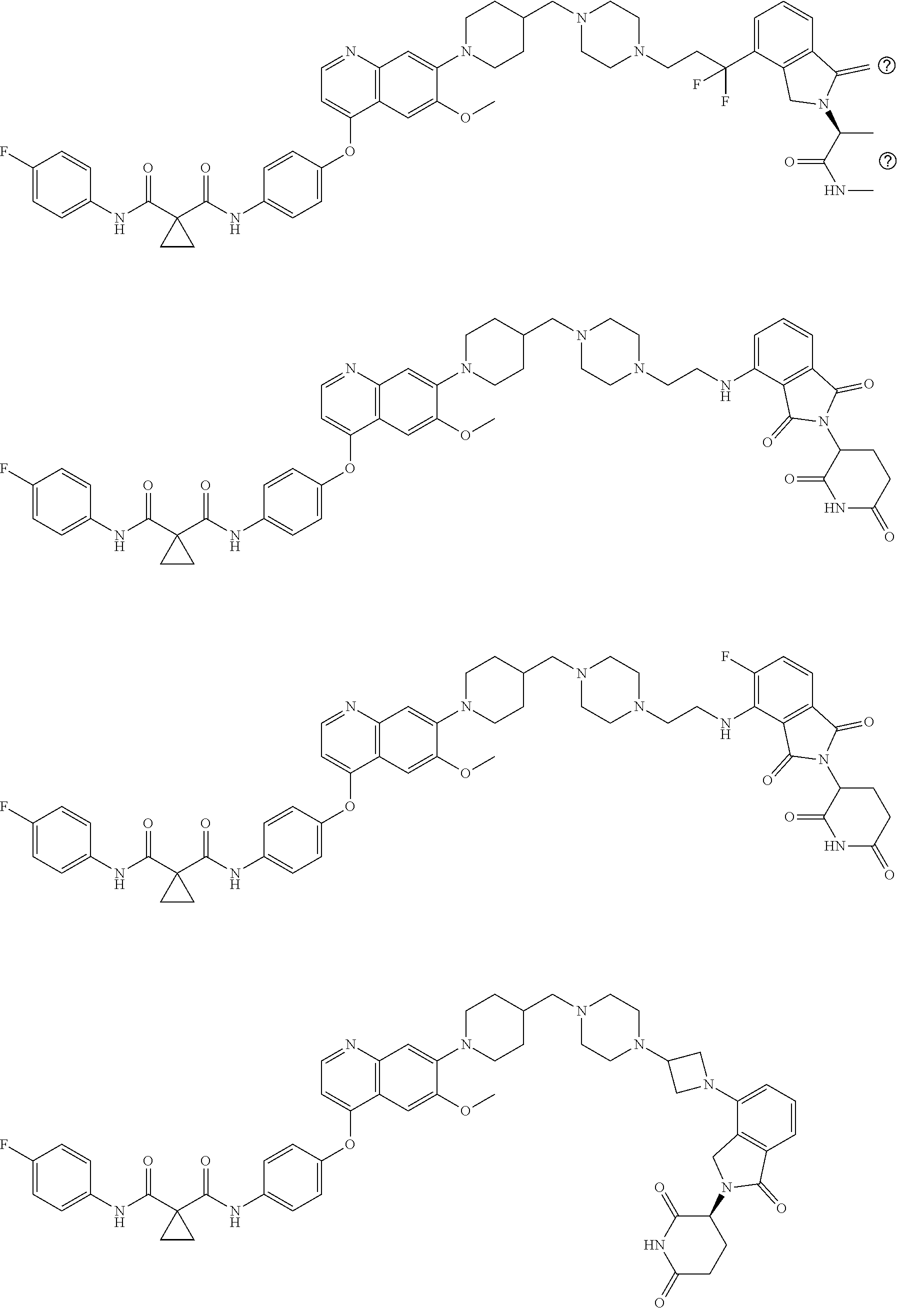 [SMILES: C=C1c2cccc(C(F)(F)CCN3CCN(CC4CCN(c5cc6nccc(Oc7ccc(NC(=O)C8(C(=O)Nc9ccc(F)cc9)CC8)cc7)c6cc5OC)CC4)CC3)c2CN1[C@@H](C)C(=O)NC ; COc1cc2c(Oc3ccc(NC(=O)C4(C(=O)Nc5ccc(F)cc5)CC4)cc3)ccnc2cc1N1CCC(CN2CCN(CCNc3cccc4c3C(=O)N(C3CCC(=O)NC3=O)C4=O)CC2)CC1 ; COc1cc2c(Oc3ccc(NC(=O)C4(C(=O)Nc5ccc(F)cc5)CC4)cc3)ccnc2cc1N1CCC(CN2CCN(CCNc3c(F)ccc4c3C(=O)N(C3CCC(=O)NC3=O)C4=O)CC2)CC1 ; COc1cc2c(Oc3ccc(NC(=O)C4(C(=O)Nc5ccc(F)cc5)CC4)cc3)ccnc2cc1N1CCC(CN2CCN(C3CN(c4cccc5c4CN([C@H]4CCC(=O)NC4=O)C5=O)C3)CC2)CC1]

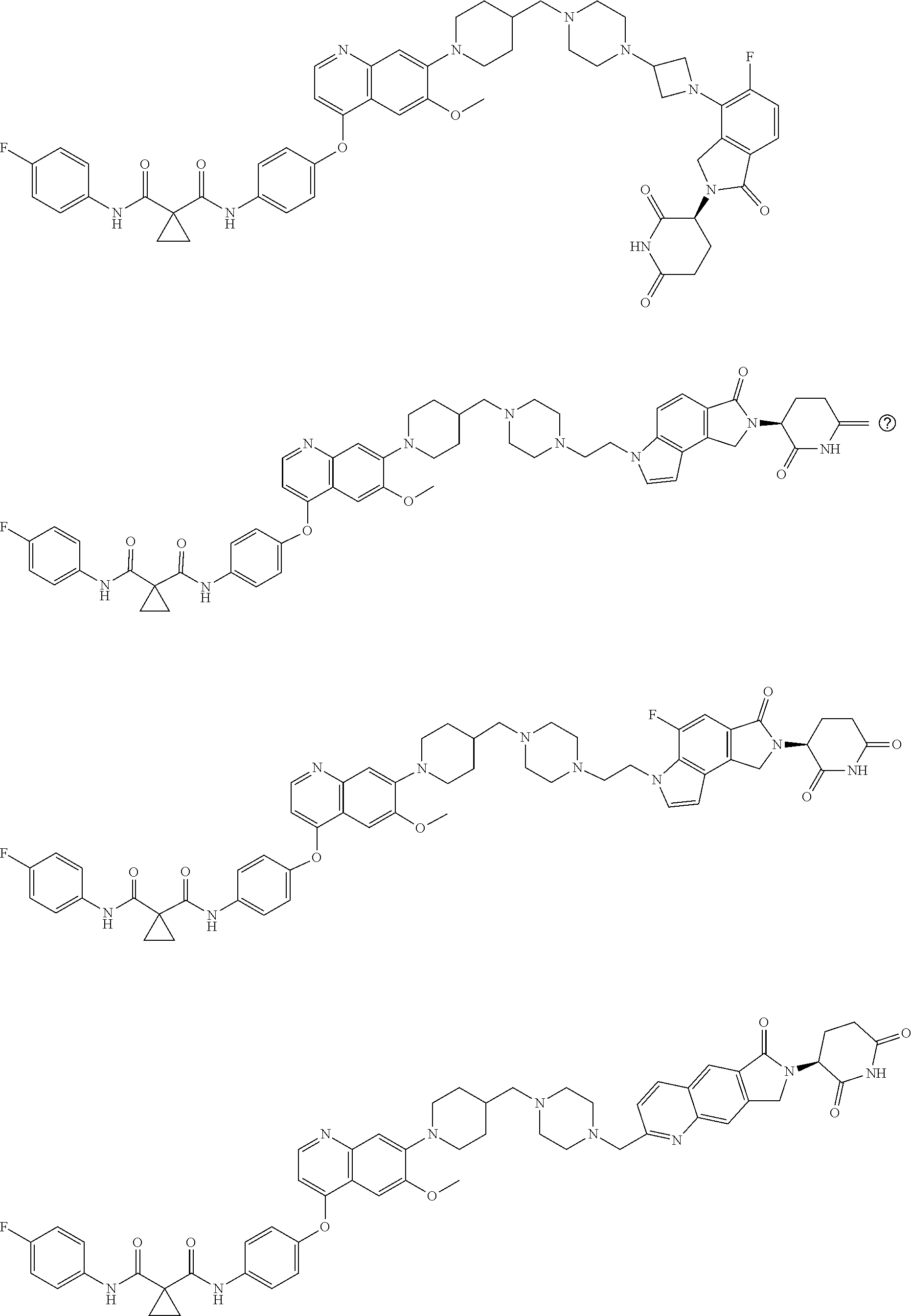

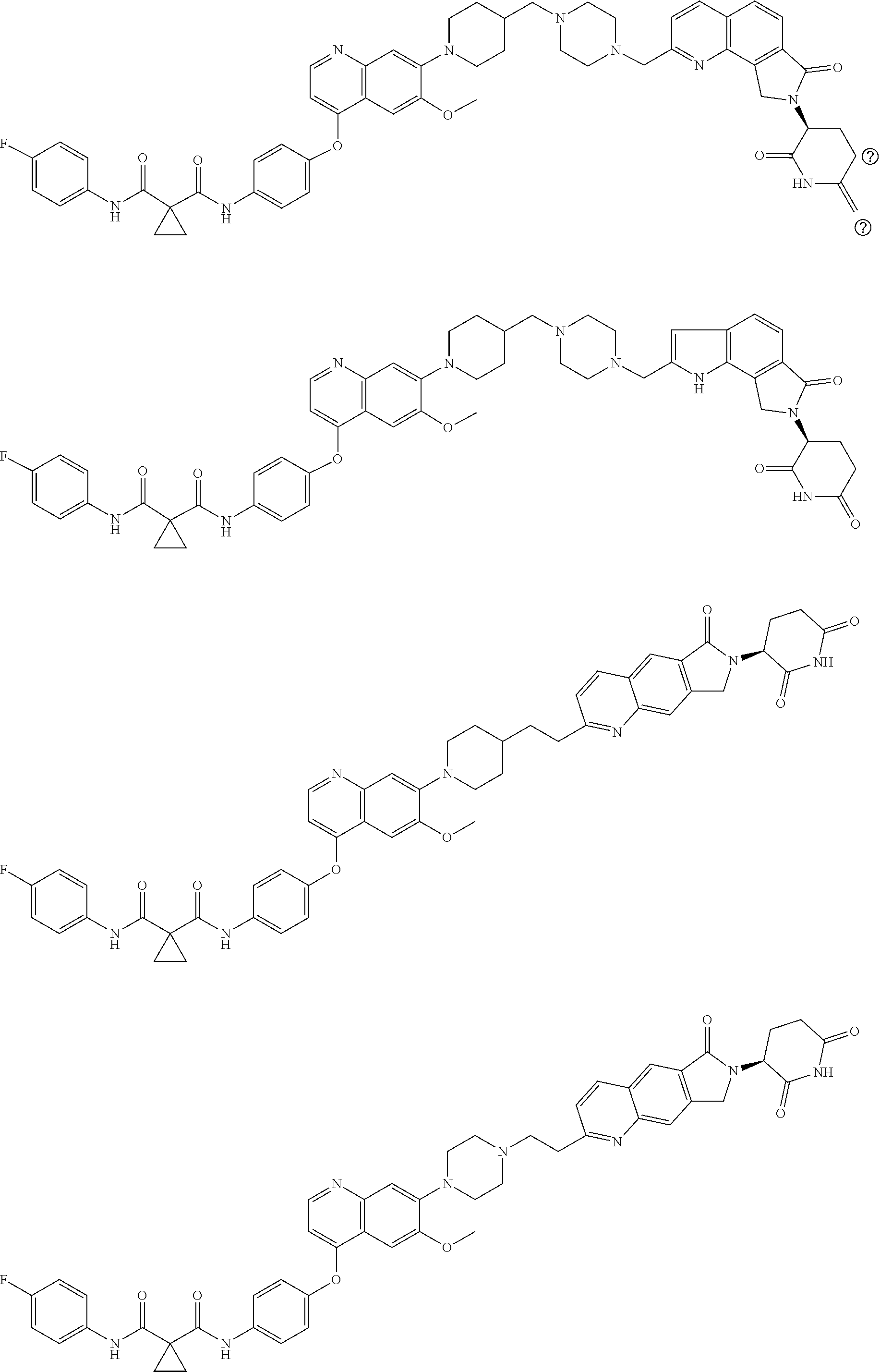 [SMILES: C=C1CC[C@H](N2Cc3c(ccc4ccc(CN5CCN(CC6CCN(c7cc8nccc(Oc9ccc(NC(=O)C%10(C(=O)Nc%11ccc(F)cc%11)CC%10)cc9)c8cc7OC)CC6)CC5)nc34)C2=O)C(=O)N1 ; COc1cc2c(Oc3ccc(NC(=O)C4(C(=O)Nc5ccc(F)cc5)CC4)cc3)ccnc2cc1N1CCC(CN2CCN(Cc3cc4ccc5c(c4[nH]3)CN([C@H]3CCC(=O)NC3=O)C5=O)CC2)CC1 ; COc1cc2c(Oc3ccc(NC(=O)C4(C(=O)Nc5ccc(F)cc5)CC4)cc3)ccnc2cc1N1CCC(CCc2ccc3cc4c(cc3n2)CN([C@H]2CCC(=O)NC2=O)C4=O)CC1 ; COc1cc2c(Oc3ccc(NC(=O)C4(C(=O)Nc5ccc(F)cc5)CC4)cc3)ccnc2cc1N1CCN(CCc2ccc3cc4c(cc3n2)CN([C@H]2CCC(=O)NC2=O)C4=O)CC1]

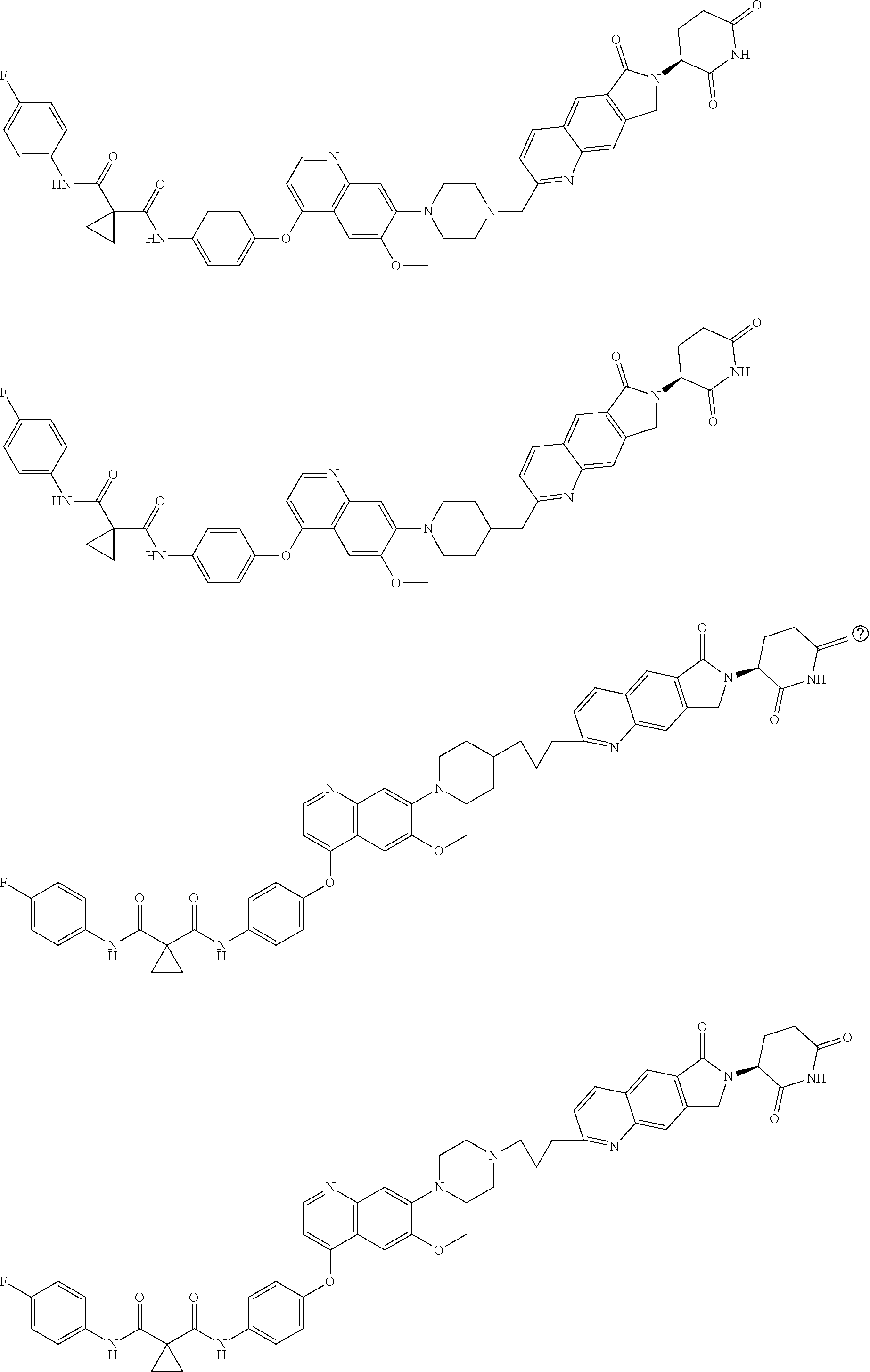 [SMILES: COc1cc2c(Oc3ccc(NC(=O)C4(C(=O)Nc5ccc(F)cc5)CC4)cc3)ccnc2cc1N1CCN(Cc2ccc3cc4c(cc3n2)CN([C@H]2CCC(=O)NC2=O)C4=O)CC1 ; COc1cc2c(Oc3ccc(NC(=O)C4(C(=O)Nc5ccc(F)cc5)CC4)cc3)ccnc2cc1N1CCC(Cc2ccc3cc4c(cc3n2)CN([C@H]2CCC(=O)NC2=O)C4=O)CC1 ; C=C1CC[C@H](N2Cc3cc4nc(CCCC5CCN(c6cc7nccc(Oc8ccc(NC(=O)C9(C(=O)Nc%10ccc(F)cc%10)CC9)cc8)c7cc6OC)CC5)ccc4cc3C2=O)C(=O)N1 ; COc1cc2c(Oc3ccc(NC(=O)C4(C(=O)Nc5ccc(F)cc5)CC4)cc3)ccnc2cc1N1CCN(CCCc2ccc3cc4c(cc3n2)CN([C@H]2CCC(=O)NC2=O)C4=O)CC1]

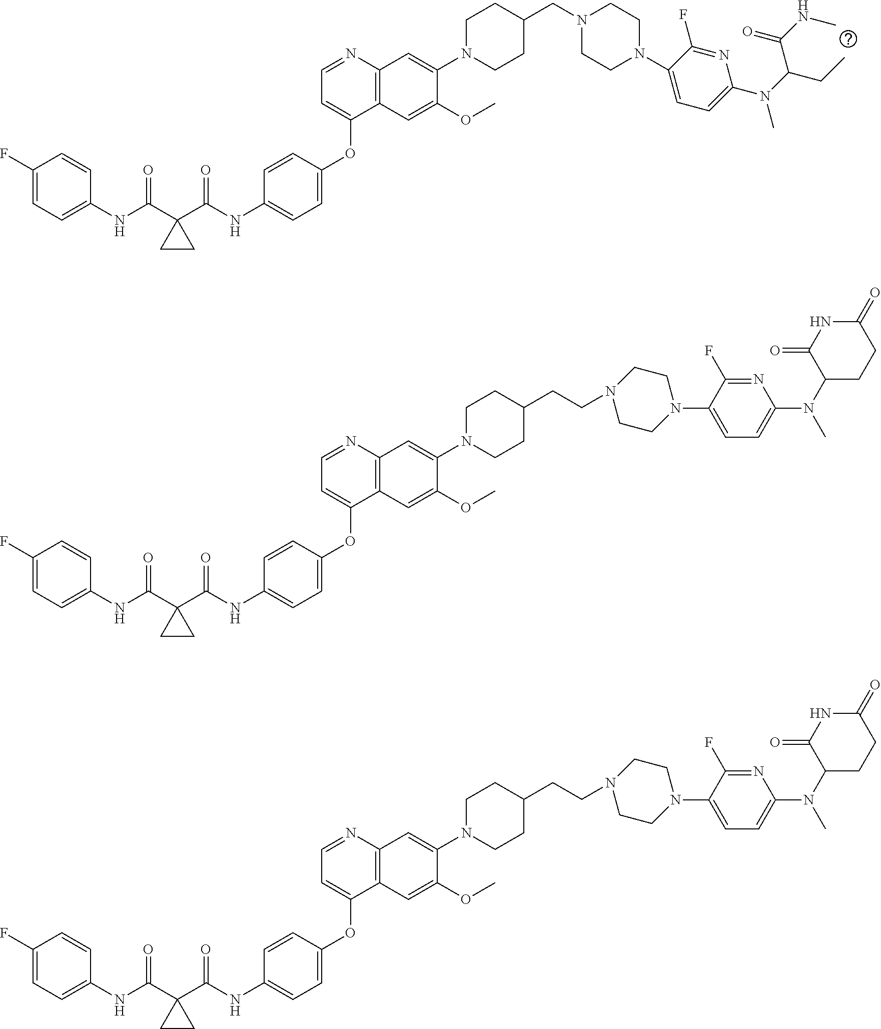 [SMILES: CCC(C(=O)NC)N(C)c1ccc(N2CCN(CC3CCN(c4cc5nccc(Oc6ccc(NC(=O)C7(C(=O)Nc8ccc(F)cc8)CC7)cc6)c5cc4OC)CC3)CC2)c(F)n1 ; COc1cc2c(Oc3ccc(NC(=O)C4(C(=O)Nc5ccc(F)cc5)CC4)cc3)ccnc2cc1N1CCC(CCN2CCN(c3ccc(N(C)C4CCC(=O)NC4=O)nc3F)CC2)CC1 ; COc1cc2c(Oc3ccc(NC(=O)C4(C(=O)Nc5ccc(F)cc5)CC4)cc3)ccnc2cc1N1CCC(CCN2CCN(c3ccc(N(C)C4CCC(=O)NC4=O)nc3F)CC2)CC1]

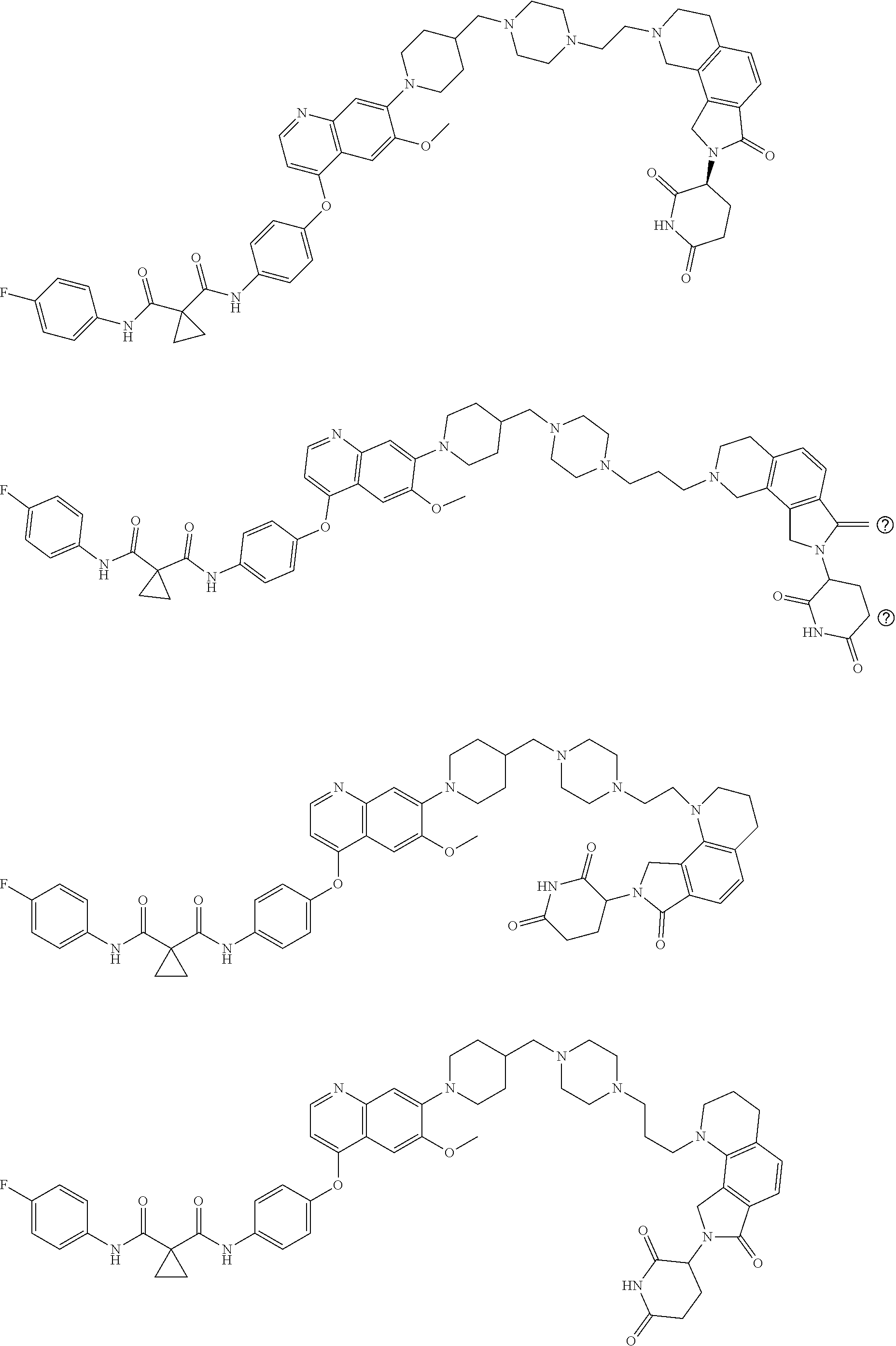 [SMILES: COc1cc2c(Oc3ccc(NC(=O)C4(C(=O)Nc5ccc(F)cc5)CC4)cc3)ccnc2cc1N1CCC(CN2CCN(CCN3CCc4ccc5c(c4C3)CN([C@H]3CCC(=O)NC3=O)C5=O)CC2)CC1 ; C=C1c2ccc3c(c2CN1C1CCC(=O)NC1=O)CN(CCCN1CCN(CC2CCN(c4cc5nccc(Oc6ccc(NC(=O)C7(C(=O)Nc8ccc(F)cc8)CC7)cc6)c5cc4OC)CC2)CC1)CC3 ; COc1cc2c(Oc3ccc(NC(=O)C4(C(=O)Nc5ccc(F)cc5)CC4)cc3)ccnc2cc1N1CCC(CN2CCN(CCN3CCCc4ccc5c(c43)CN(C3CCC(=O)NC3=O)C5=O)CC2)CC1 ; COc1cc2c(Oc3ccc(NC(=O)C4(C(=O)Nc5ccc(F)cc5)CC4)cc3)ccnc2cc1N1CCC(CN2CCN(CCCN3CCCc4ccc5c(c43)CN(C3CCC(=O)NC3=O)C5=O)CC2)CC1]

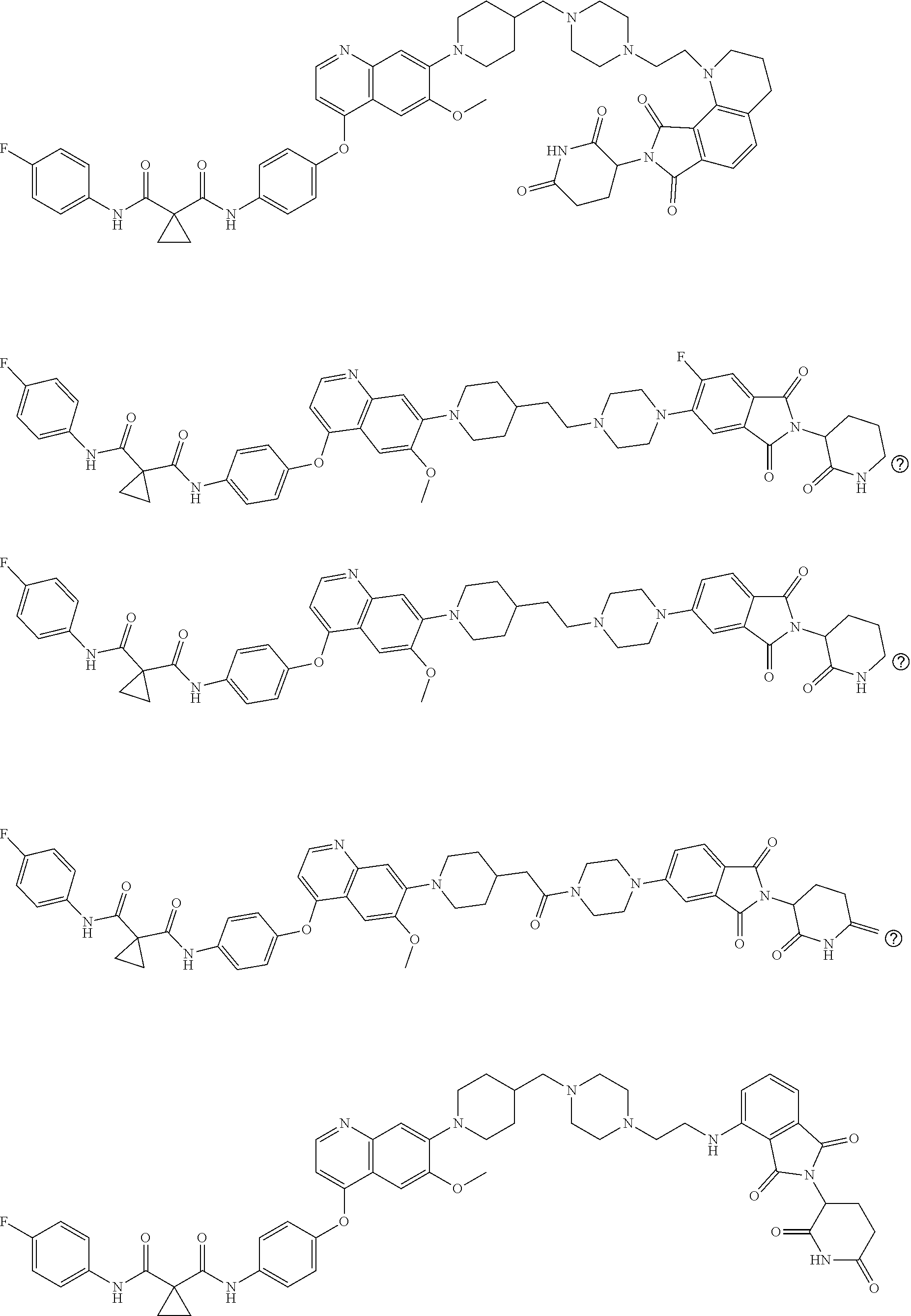

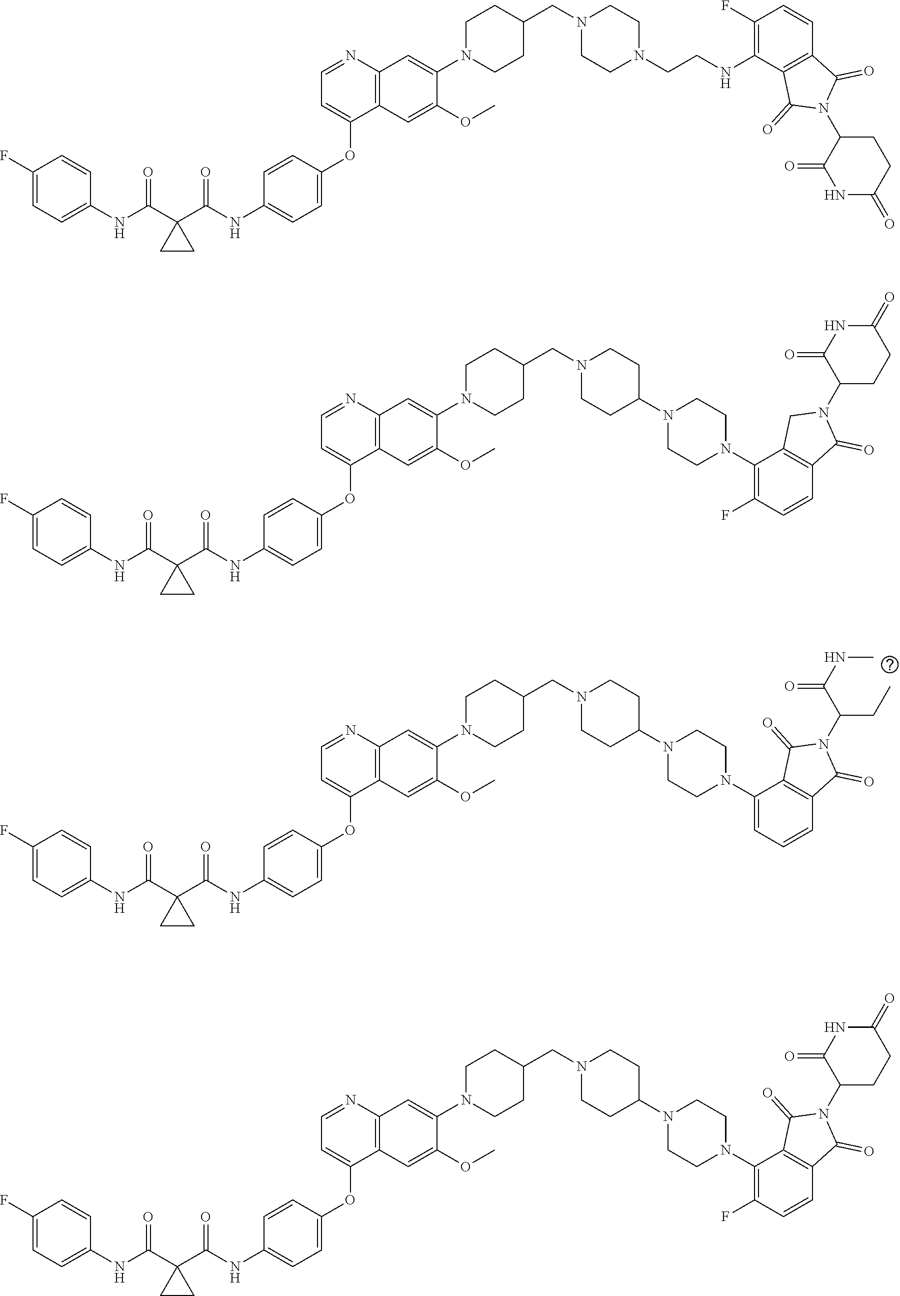

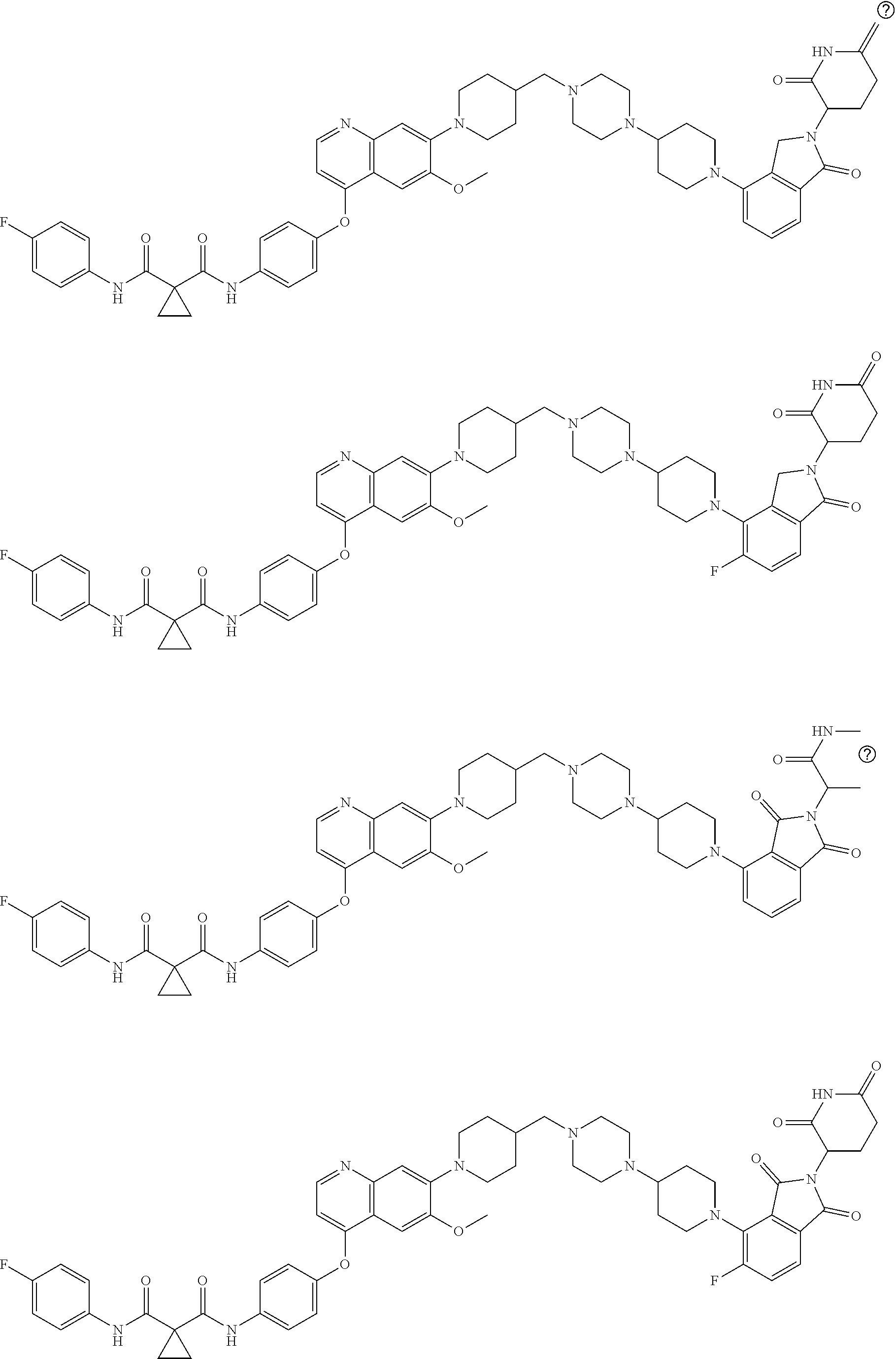

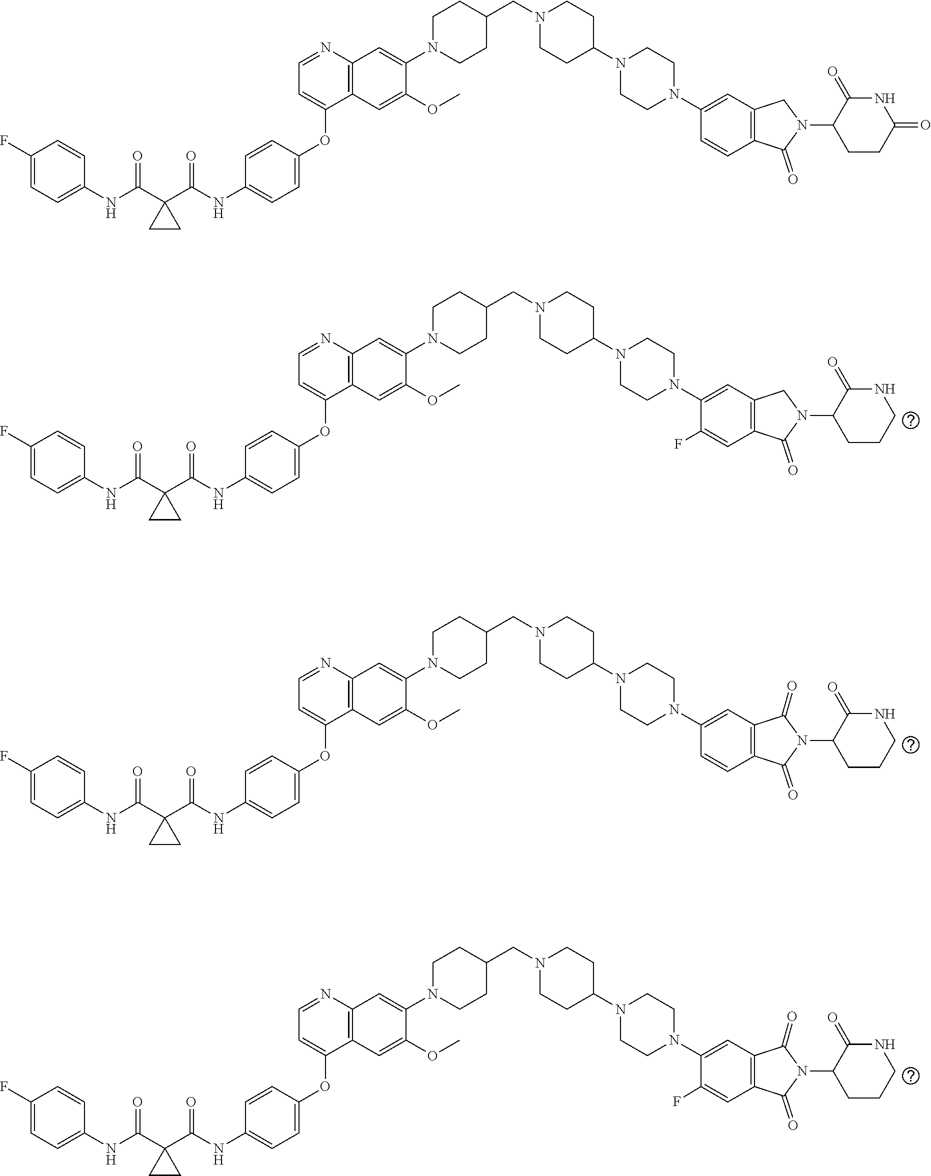

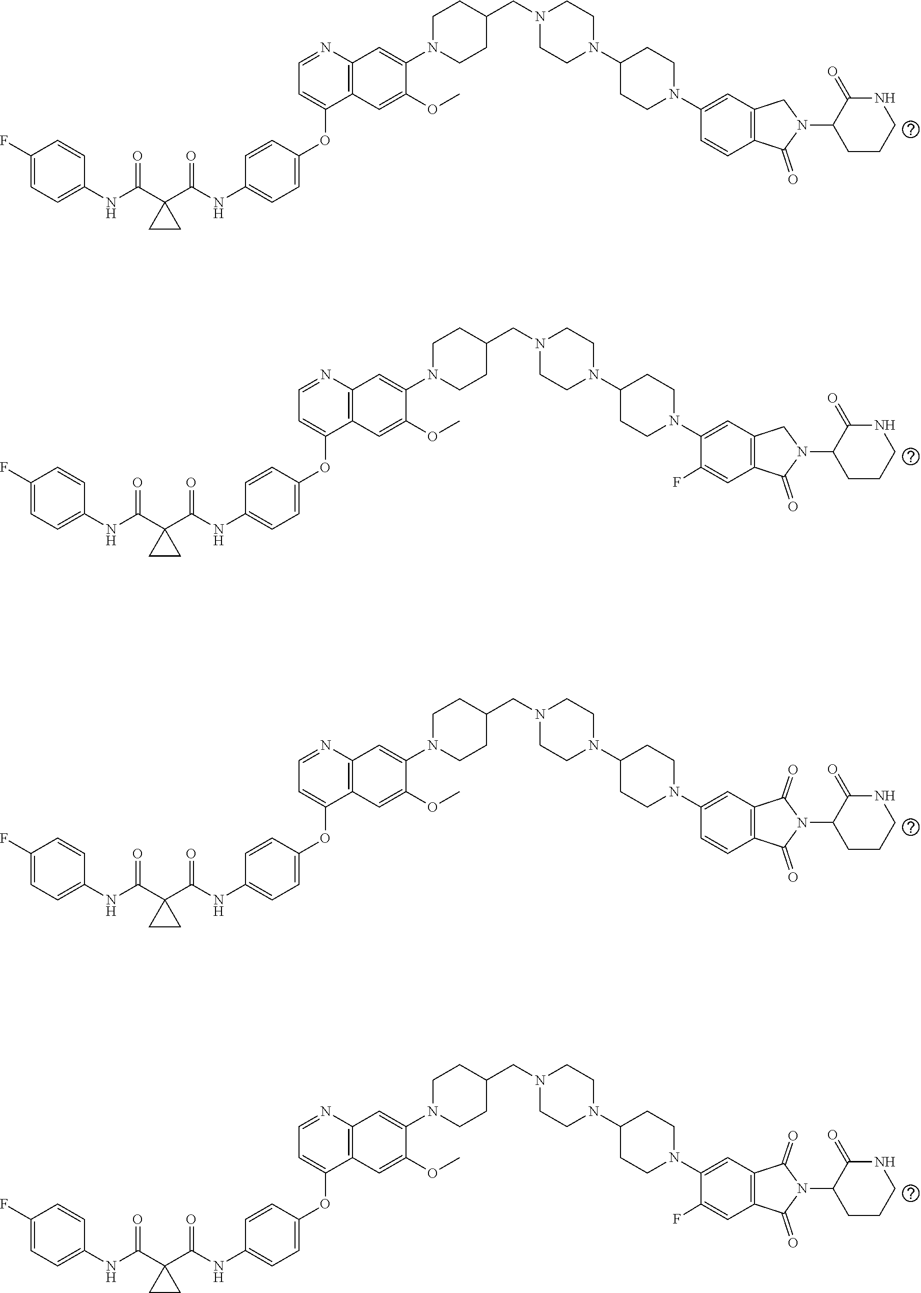 [SMILES: COc1cc2c(Oc3ccc(NC(=O)C4(C(=O)Nc5ccc(F)cc5)CC4)cc3)ccnc2cc1N1CCC(CN2CCN(C3CCN(c4ccc5c(c4)CN(C4CCCNC4=O)C5=O)CC3)CC2)CC1 ; COc1cc2c(Oc3ccc(NC(=O)C4(C(=O)Nc5ccc(F)cc5)CC4)cc3)ccnc2cc1N1CCC(CN2CCN(C3CCN(c4cc5c(cc4F)C(=O)N(C4CCCNC4=O)C5)CC3)CC2)CC1 ; COc1cc2c(Oc3ccc(NC(=O)C4(C(=O)Nc5ccc(F)cc5)CC4)cc3)ccnc2cc1N1CCC(CN2CCN(C3CCN(c4ccc5c(c4)C(=O)N(C4CCCNC4=O)C5=O)CC3)CC2)CC1 ; COc1cc2c(Oc3ccc(NC(=O)C4(C(=O)Nc5ccc(F)cc5)CC4)cc3)ccnc2cc1N1CCC(CN2CCN(C3CCN(c4cc5c(cc4F)C(=O)N(C4CCCNC4=O)C5=O)CC3)CC2)CC1]

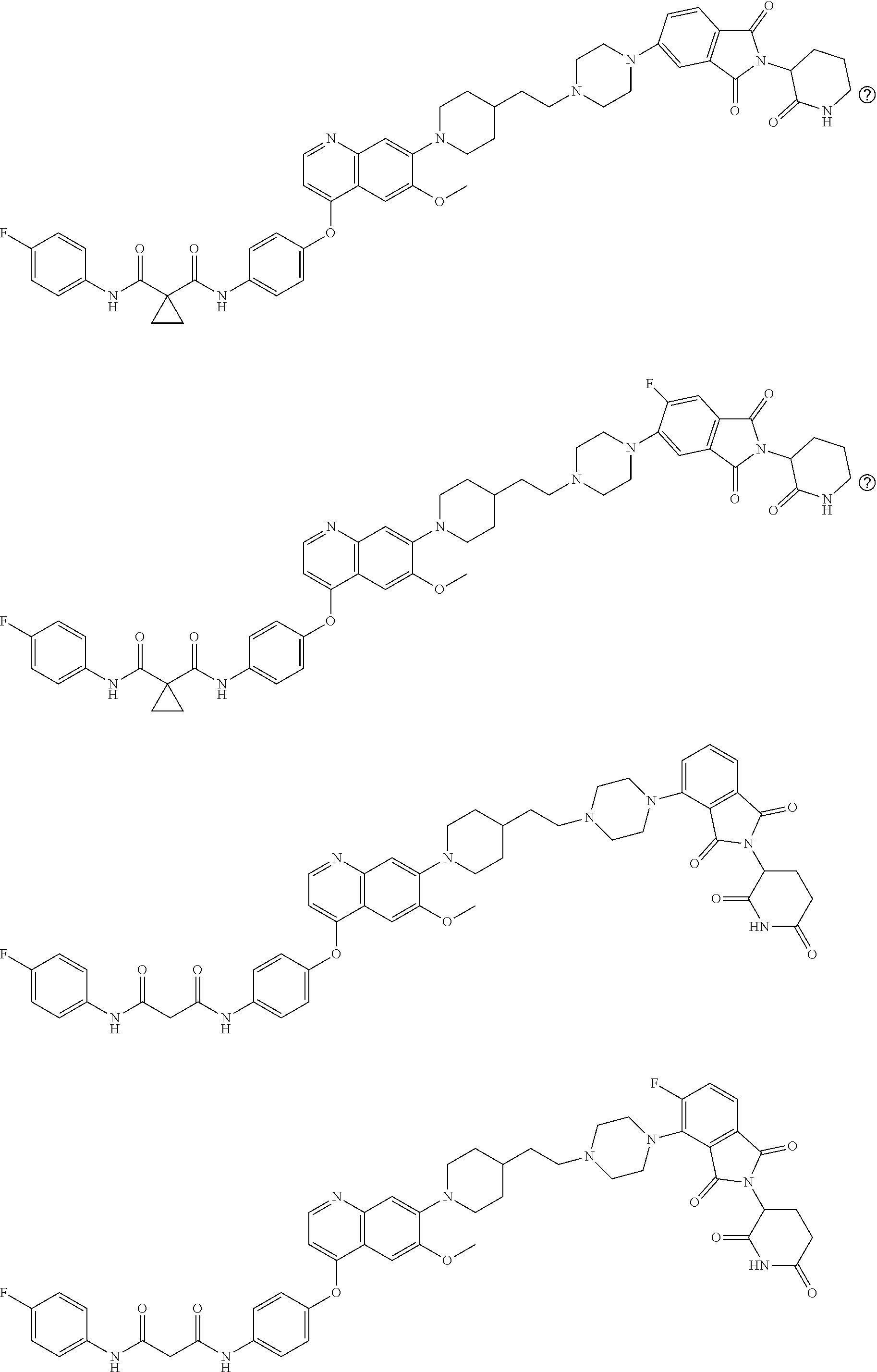 [SMILES: COc1cc2c(Oc3ccc(NC(=O)C4(C(=O)Nc5ccc(F)cc5)CC4)cc3)ccnc2cc1N1CCC(CCN2CCN(c3ccc4c(c3)C(=O)N(C3CCCNC3=O)C4=O)CC2)CC1 ; COc1cc2c(Oc3ccc(NC(=O)C4(C(=O)Nc5ccc(F)cc5)CC4)cc3)ccnc2cc1N1CCC(CCN2CCN(c3cc4c(cc3F)C(=O)N(C3CCCNC3=O)C4=O)CC2)CC1 ; COc1cc2c(Oc3ccc(NC(=O)CC(=O)Nc4ccc(F)cc4)cc3)ccnc2cc1N1CCC(CCN2CCN(c3cccc4c3C(=O)N(C3CCC(=O)NC3=O)C4=O)CC2)CC1 ; COc1cc2c(Oc3ccc(NC(=O)CC(=O)Nc4ccc(F)cc4)cc3)ccnc2cc1N1CCC(CCN2CCN(c3c(F)ccc4c3C(=O)N(C3CCC(=O)NC3=O)C4=O)CC2)CC1]

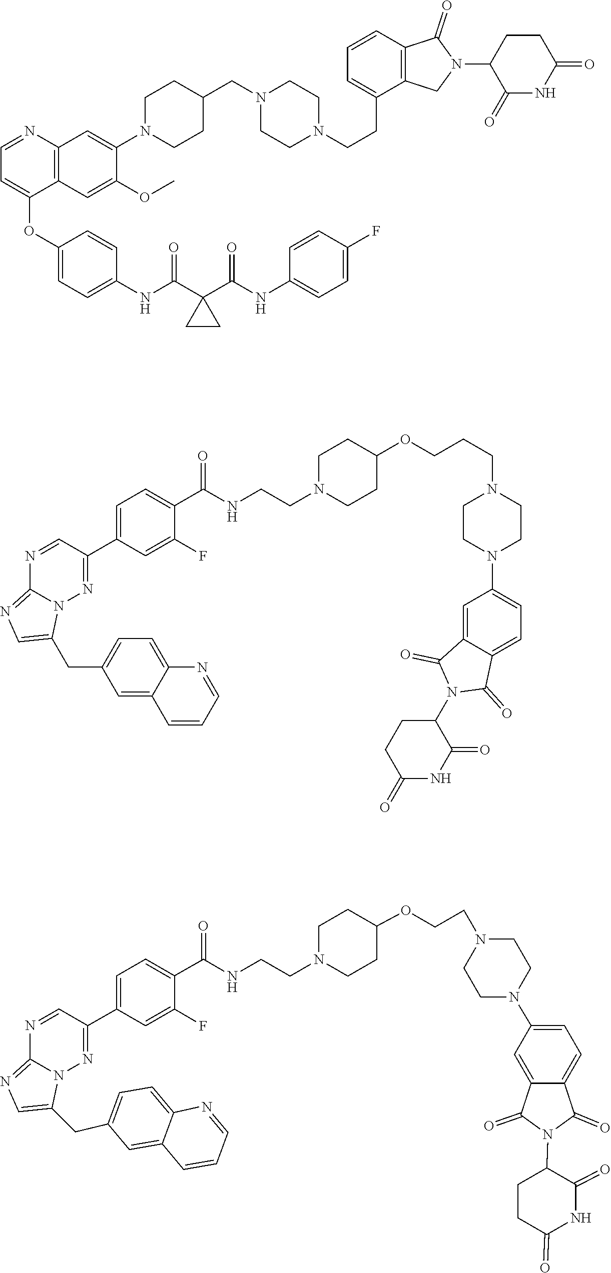 [SMILES: COc1cc2c(Oc3ccc(NC(=O)C4(C(=O)Nc5ccc(F)cc5)CC4)cc3)ccnc2cc1N1CCC(CN2CCN(CCc3cccc4c3CN(C3CCC(=O)NC3=O)C4=O)CC2)CC1 ; O=C1CCC(N2C(=O)c3ccc(N4CCN(CCCOC5CCN(CCNC(=O)c6ccc(-c7cnc8ncc(Cc9ccc%10ncccc%10c9)n8n7)cc6F)CC5)CC4)cc3C2=O)C(=O)N1 ; O=C1CCC(N2C(=O)c3ccc(N4CCN(CCOC5CCN(CCNC(=O)c6ccc(-c7cnc8ncc(Cc9ccc%10ncccc%10c9)n8n7)cc6F)CC5)CC4)cc3C2=O)C(=O)N1]

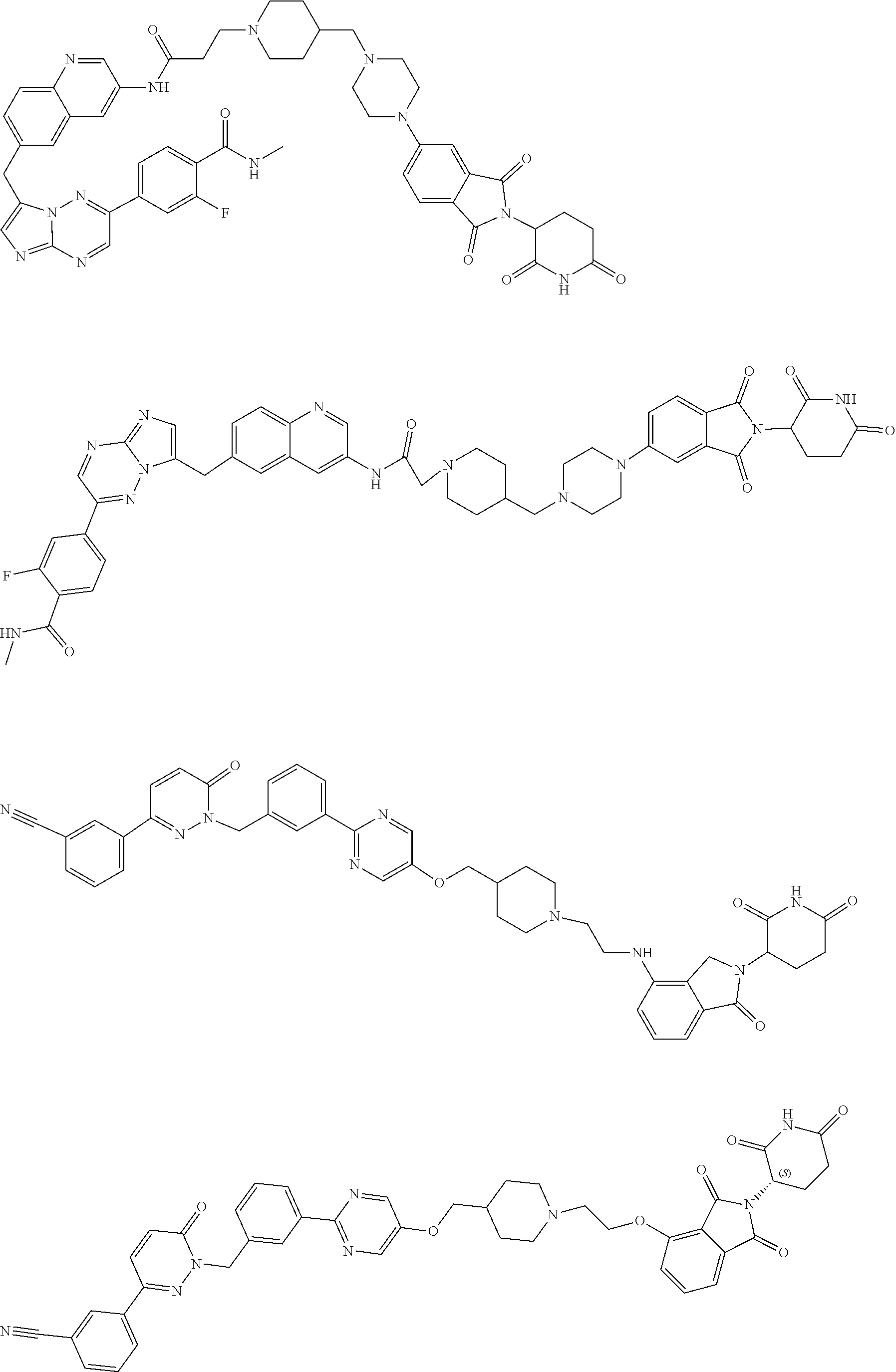

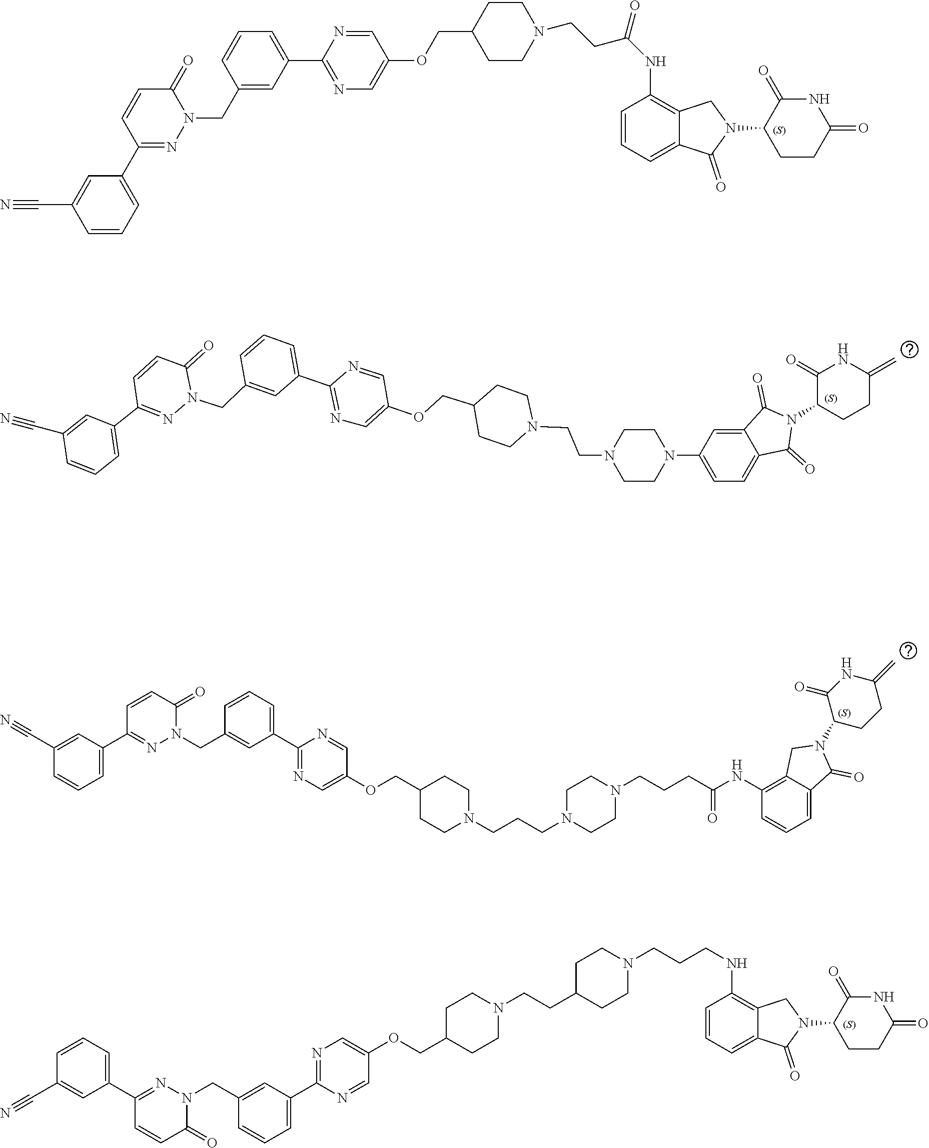

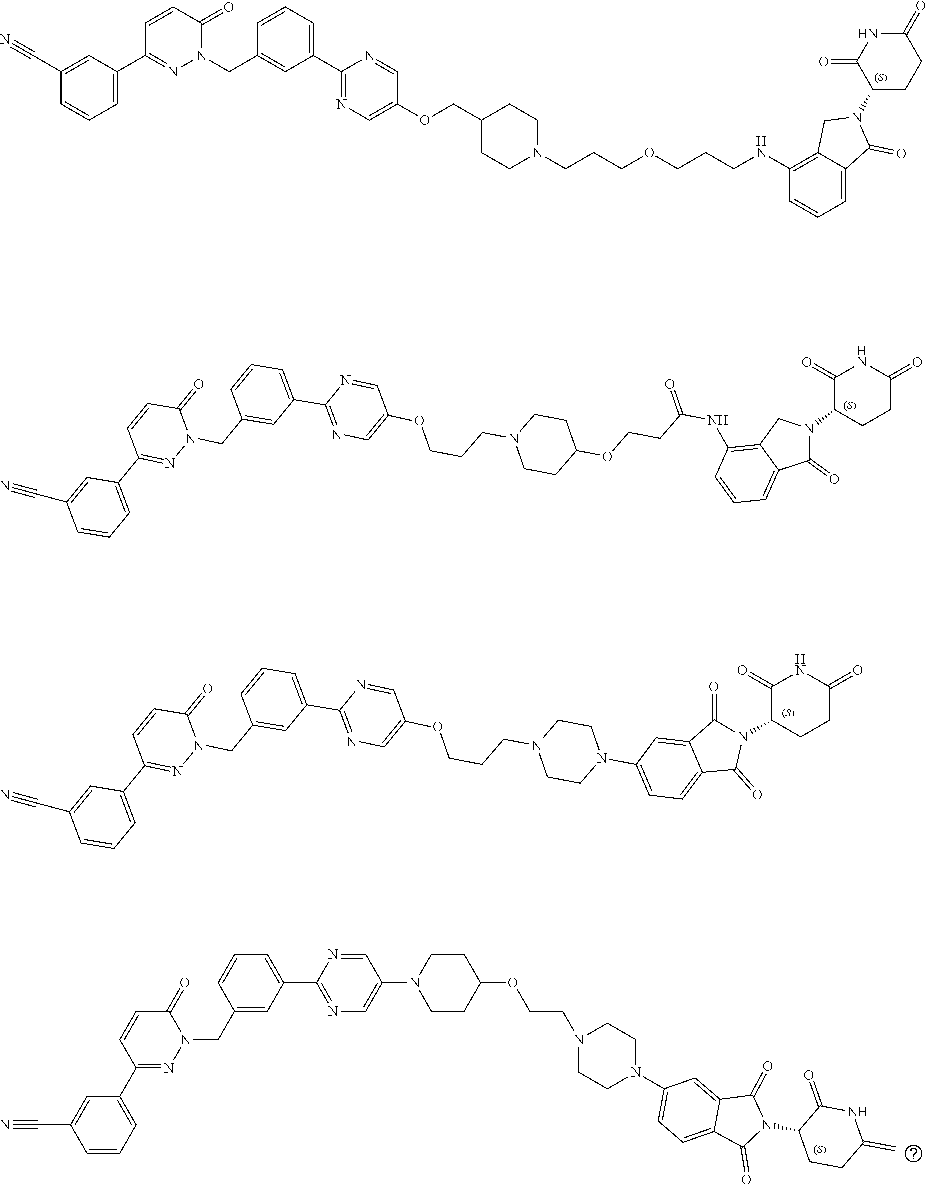

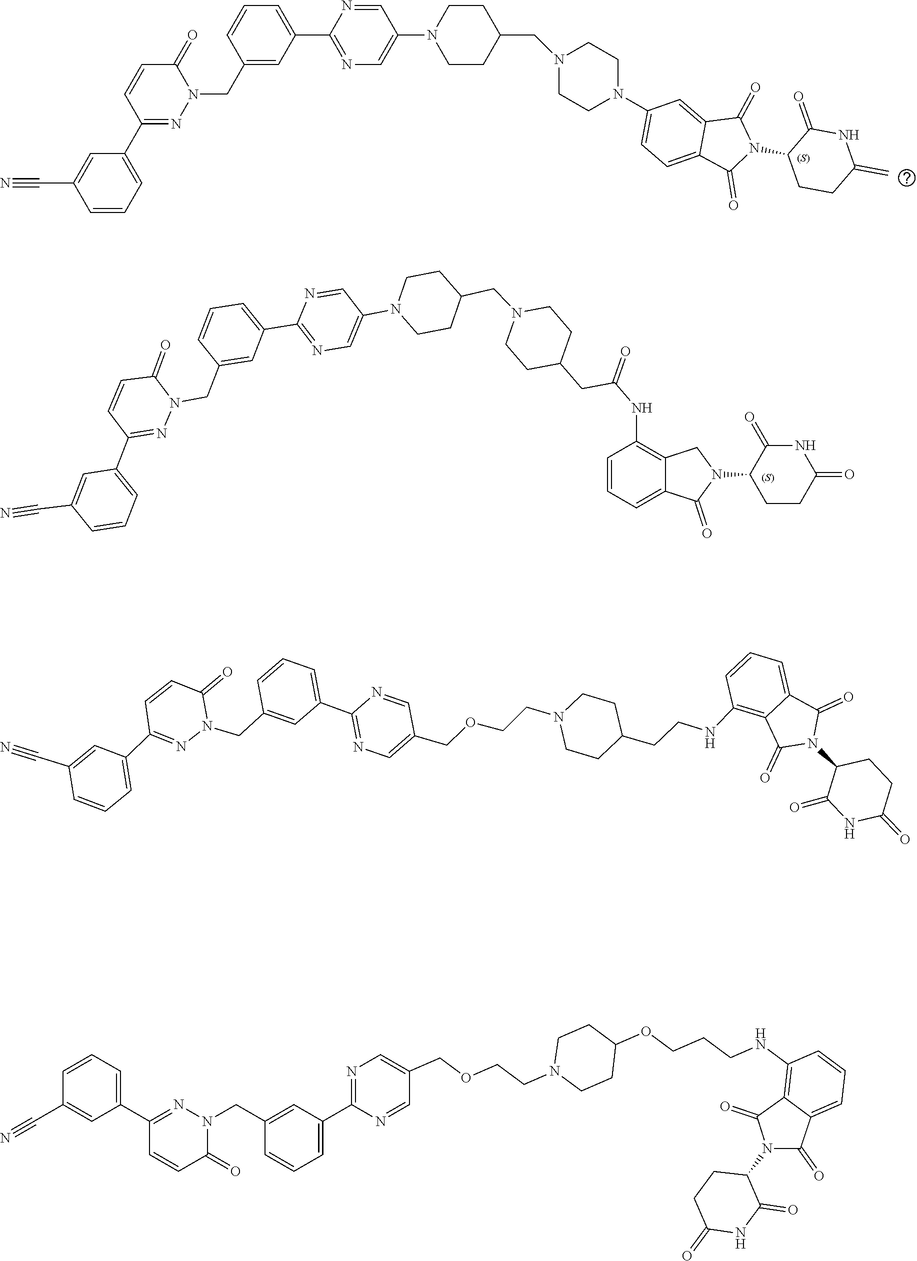

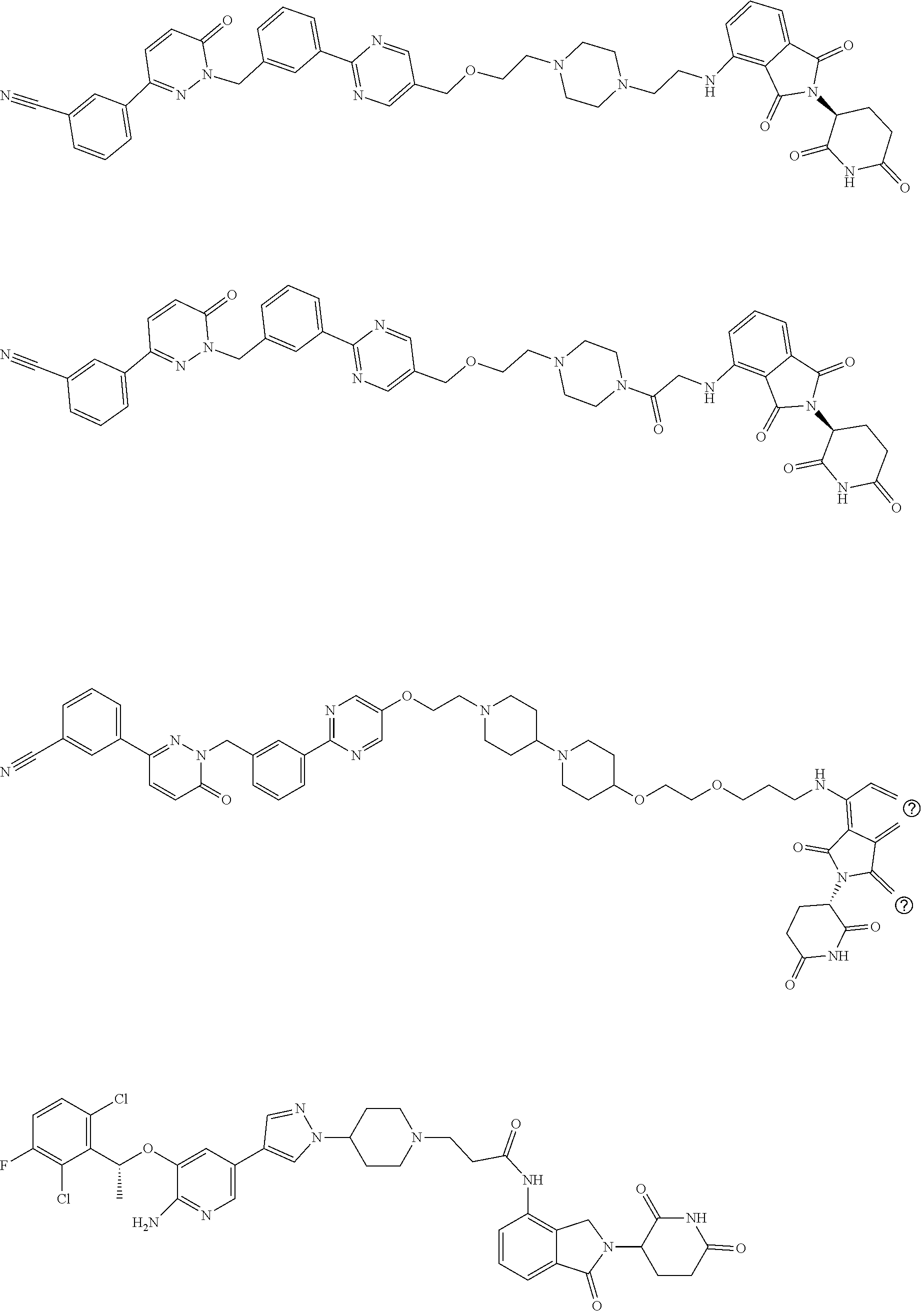 [SMILES: N#Cc1cccc(-c2ccc(=O)n(Cc3cccc(-c4ncc(COCCN5CCN(CCNc6cccc7c6C(=O)N([C@H]6CCC(=O)NC6=O)C7=O)CC5)cn4)c3)n2)c1 ; N#Cc1cccc(-c2ccc(=O)n(Cc3cccc(-c4ncc(COCCN5CCN(C(=O)CNc6cccc7c6C(=O)N([C@H]6CCC(=O)NC6=O)C7=O)CC5)cn4)c3)n2)c1 ; C=C/C(NCCCOCCOC1CCN(C2CCN(CCOc3cnc(-c4cccc(Cn5nc(-c6cccc(C#N)c6)ccc5=O)c4)nc3)CC2)CC1)=C1\C(=C)C(=C)N([C@H]2CCC(=O)NC2=O)C1=O ; C[C@@H](Oc1cc(-c2cnn(C3CCN(CCC(=O)Nc4cccc5c4CN(C4CCC(=O)NC4=O)C5=O)CC3)c2)cnc1N)c1c(Cl)ccc(F)c1Cl]

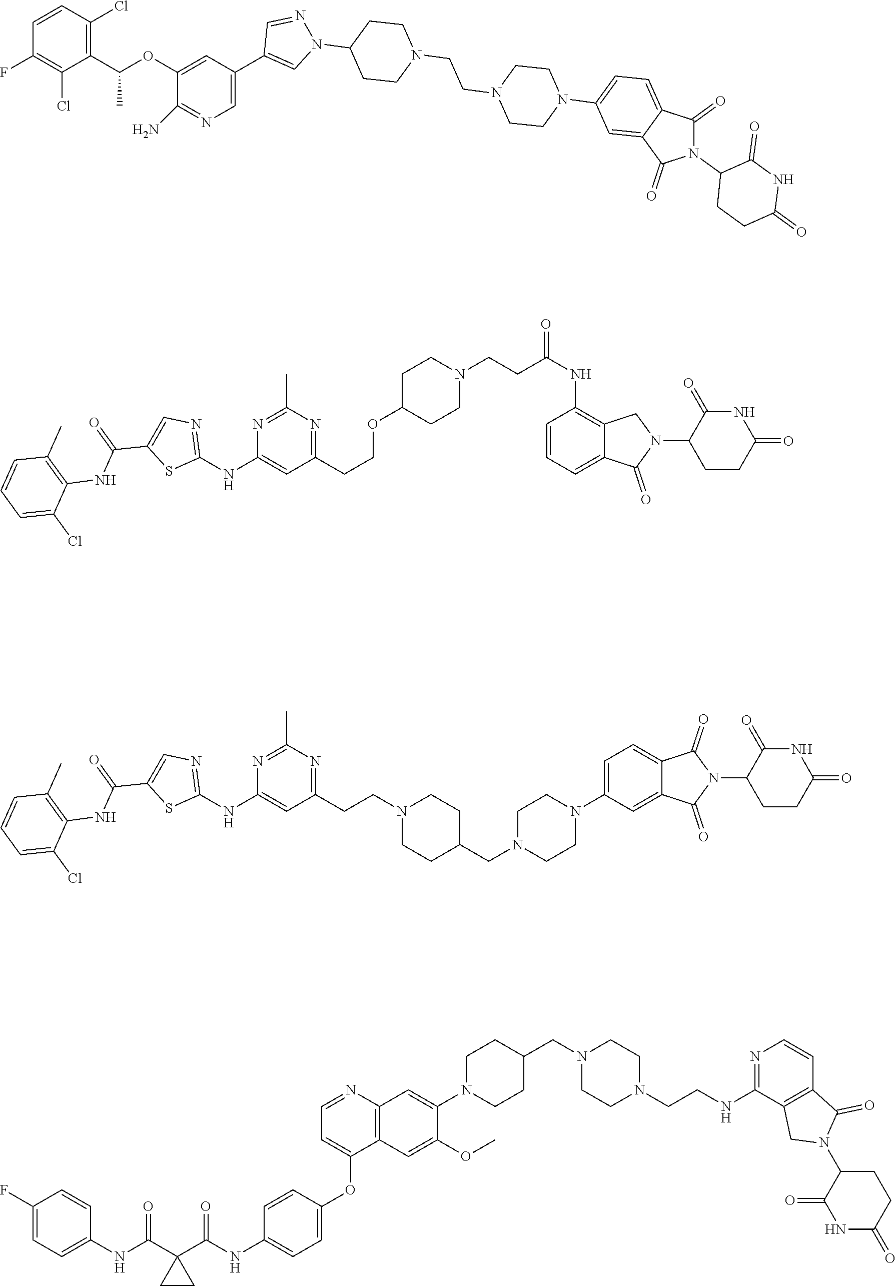 [SMILES: C[C@@H](Oc1cc(-c2cnn(C3CCN(CCN4CCN(c5ccc6c(c5)C(=O)N(C5CCC(=O)NC5=O)C6=O)CC4)CC3)c2)cnc1N)c1c(Cl)ccc(F)c1Cl ; Cc1nc(CCOC2CCN(CCC(=O)Nc3cccc4c3CN(C3CCC(=O)NC3=O)C4=O)CC2)cc(Nc2ncc(C(=O)Nc3c(C)cccc3Cl)s2)n1 ; Cc1nc(CCN2CCC(CN3CCN(c4ccc5c(c4)C(=O)N(C4CCC(=O)NC4=O)C5=O)CC3)CC2)cc(Nc2ncc(C(=O)Nc3c(C)cccc3Cl)s2)n1 ; COc1cc2c(Oc3ccc(NC(=O)C4(C(=O)Nc5ccc(F)cc5)CC4)cc3)ccnc2cc1N1CCC(CN2CCN(CCNc3nccc4c3CN(C3CCC(=O)NC3=O)C4=O)CC2)CC1]

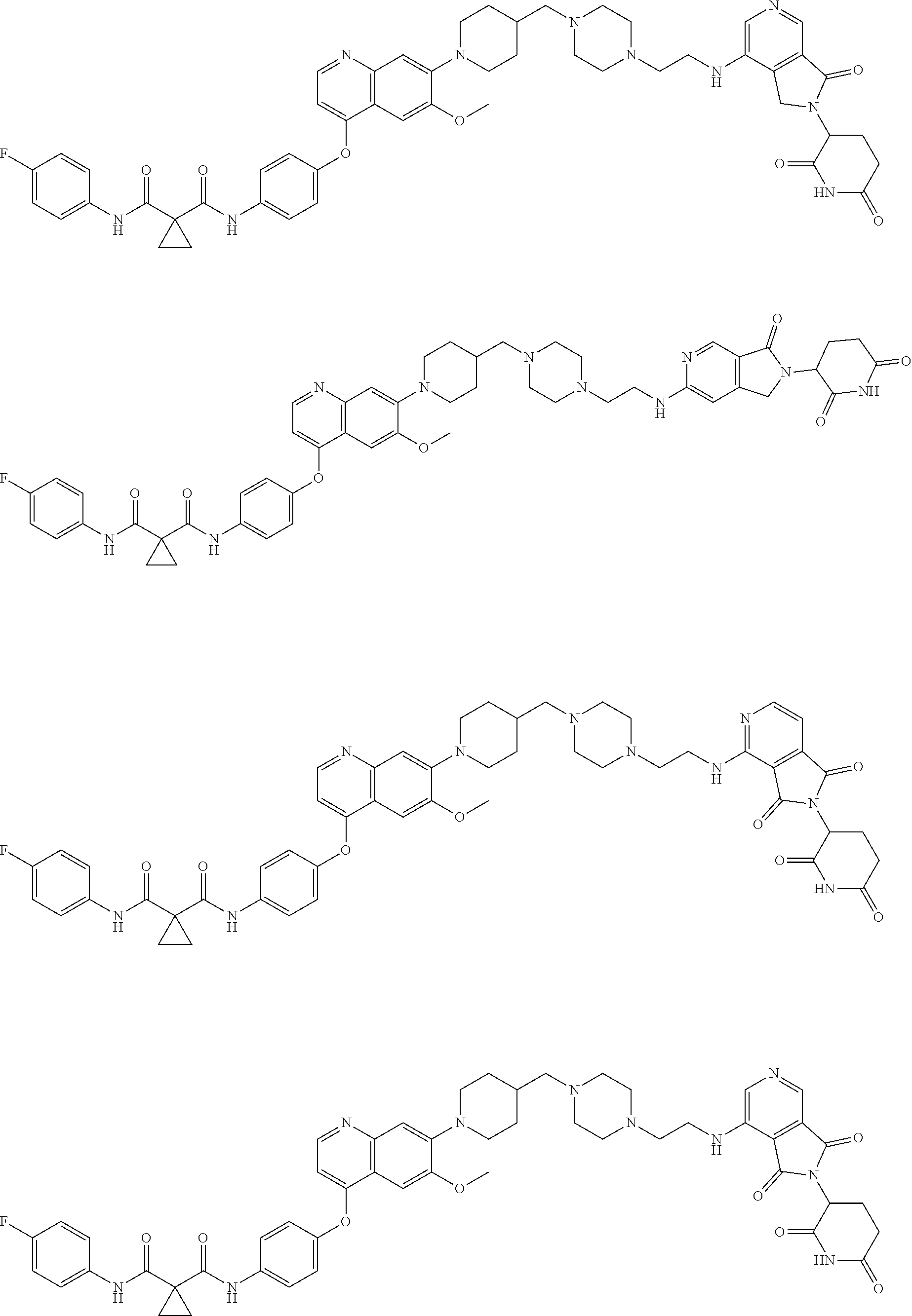 [SMILES: COc1cc2c(Oc3ccc(NC(=O)C4(C(=O)Nc5ccc(F)cc5)CC4)cc3)ccnc2cc1N1CCC(CN2CCN(CCNc3cncc4c3CN(C3CCC(=O)NC3=O)C4=O)CC2)CC1 ; COc1cc2c(Oc3ccc(NC(=O)C4(C(=O)Nc5ccc(F)cc5)CC4)cc3)ccnc2cc1N1CCC(CN2CCN(CCNc3cc4c(cn3)C(=O)N(C3CCC(=O)NC3=O)C4)CC2)CC1 ; COc1cc2c(Oc3ccc(NC(=O)C4(C(=O)Nc5ccc(F)cc5)CC4)cc3)ccnc2cc1N1CCC(CN2CCN(CCNc3nccc4c3C(=O)N(C3CCC(=O)NC3=O)C4=O)CC2)CC1 ; COc1cc2c(Oc3ccc(NC(=O)C4(C(=O)Nc5ccc(F)cc5)CC4)cc3)ccnc2cc1N1CCC(CN2CCN(CCNc3cncc4c3C(=O)N(C3CCC(=O)NC3=O)C4=O)CC2)CC1]

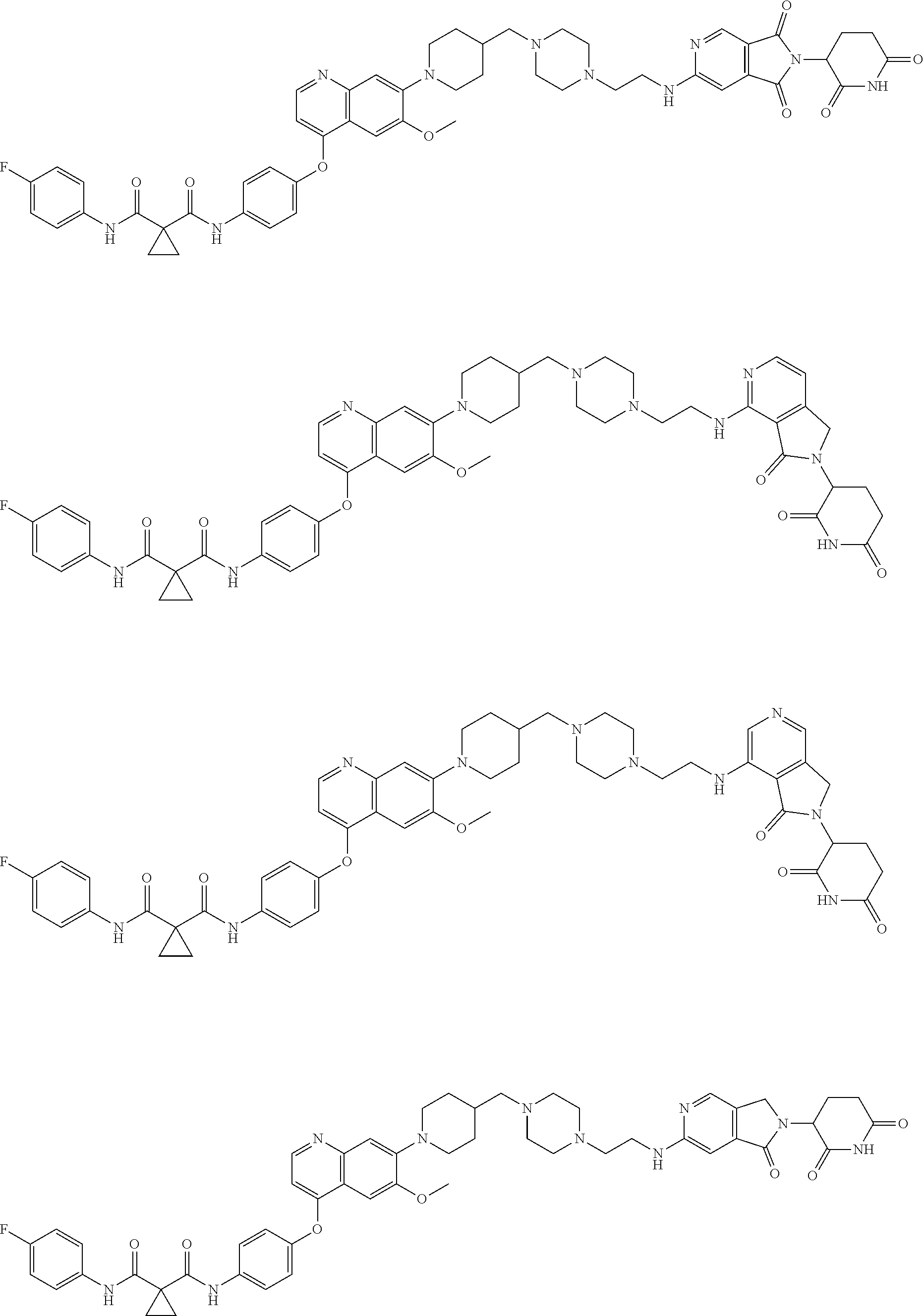 [SMILES: COc1cc2c(Oc3ccc(NC(=O)C4(C(=O)Nc5ccc(F)cc5)CC4)cc3)ccnc2cc1N1CCC(CN2CCN(CCNc3cc4c(cn3)C(=O)N(C3CCC(=O)NC3=O)C4=O)CC2)CC1 ; COc1cc2c(Oc3ccc(NC(=O)C4(C(=O)Nc5ccc(F)cc5)CC4)cc3)ccnc2cc1N1CCC(CN2CCN(CCNc3nccc4c3C(=O)N(C3CCC(=O)NC3=O)C4)CC2)CC1 ; COc1cc2c(Oc3ccc(NC(=O)C4(C(=O)Nc5ccc(F)cc5)CC4)cc3)ccnc2cc1N1CCC(CN2CCN(CCNc3cncc4c3C(=O)N(C3CCC(=O)NC3=O)C4)CC2)CC1 ; COc1cc2c(Oc3ccc(NC(=O)C4(C(=O)Nc5ccc(F)cc5)CC4)cc3)ccnc2cc1N1CCC(CN2CCN(CCNc3cc4c(cn3)CN(C3CCC(=O)NC3=O)C4=O)CC2)CC1]

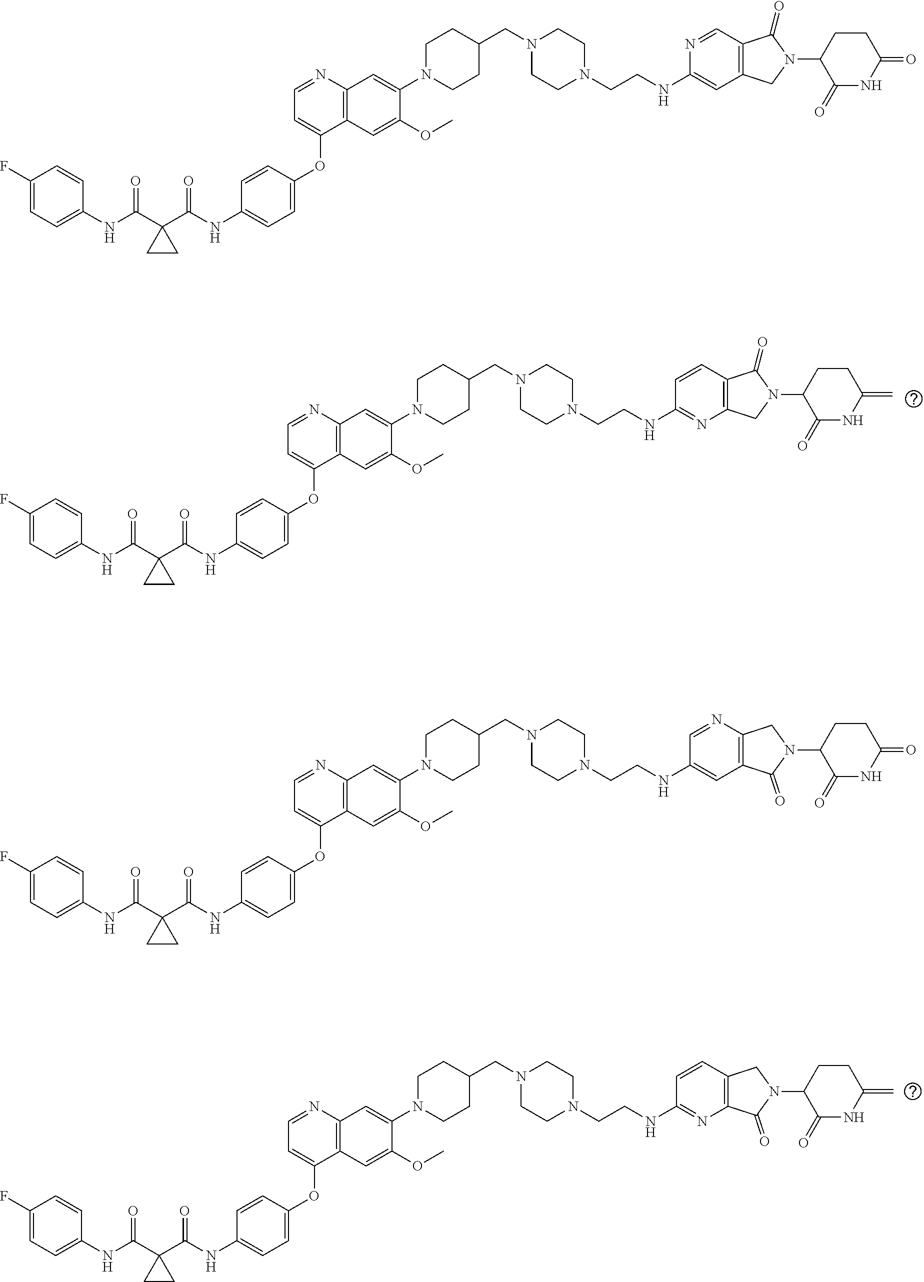 [SMILES: COc1cc2c(Oc3ccc(NC(=O)C4(C(=O)Nc5ccc(F)cc5)CC4)cc3)ccnc2cc1N1CCC(CN2CCN(CCNc3cc4c(cn3)C(=O)N(C3CCC(=O)NC3=O)C4)CC2)CC1 ; C=C1CCC(N2Cc3nc(NCCN4CCN(CC5CCN(c6cc7nccc(Oc8ccc(NC(=O)C9(C(=O)Nc%10ccc(F)cc%10)CC9)cc8)c7cc6OC)CC5)CC4)ccc3C2=O)C(=O)N1 ; COc1cc2c(Oc3ccc(NC(=O)C4(C(=O)Nc5ccc(F)cc5)CC4)cc3)ccnc2cc1N1CCC(CN2CCN(CCNc3cnc4c(c3)C(=O)N(C3CCC(=O)NC3=O)C4)CC2)CC1 ; C=C1CCC(N2Cc3ccc(NCCN4CCN(CC5CCN(c6cc7nccc(Oc8ccc(NC(=O)C9(C(=O)Nc%10ccc(F)cc%10)CC9)cc8)c7cc6OC)CC5)CC4)nc3C2=O)C(=O)N1]

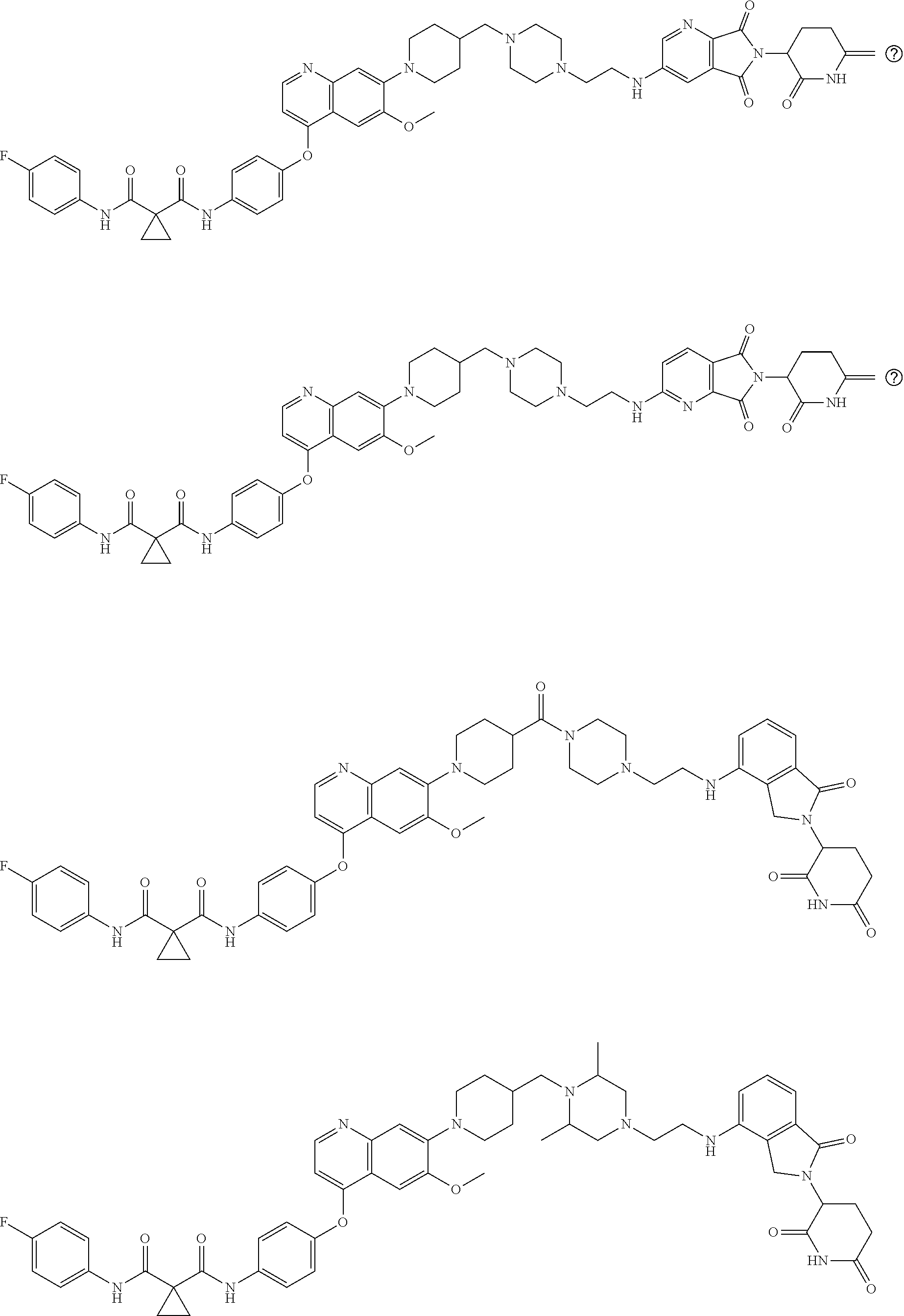 [SMILES: C=C1CCC(N2C(=O)c3cc(NCCN4CCN(CC5CCN(c6cc7nccc(Oc8ccc(NC(=O)C9(C(=O)Nc%10ccc(F)cc%10)CC9)cc8)c7cc6OC)CC5)CC4)cnc3C2=O)C(=O)N1 ; C=C1CCC(N2C(=O)c3ccc(NCCN4CCN(CC5CCN(c6cc7nccc(Oc8ccc(NC(=O)C9(C(=O)Nc%10ccc(F)cc%10)CC9)cc8)c7cc6OC)CC5)CC4)nc3C2=O)C(=O)N1 ; COc1cc2c(Oc3ccc(NC(=O)C4(C(=O)Nc5ccc(F)cc5)CC4)cc3)ccnc2cc1N1CCC(C(=O)N2CCN(CCNc3cccc4c3CN(C3CCC(=O)NC3=O)C4=O)CC2)CC1 ; COc1cc2c(Oc3ccc(NC(=O)C4(C(=O)Nc5ccc(F)cc5)CC4)cc3)ccnc2cc1N1CCC(CN2C(C)CN(CCNc3cccc4c3CN(C3CCC(=O)NC3=O)C4=O)CC2C)CC1]

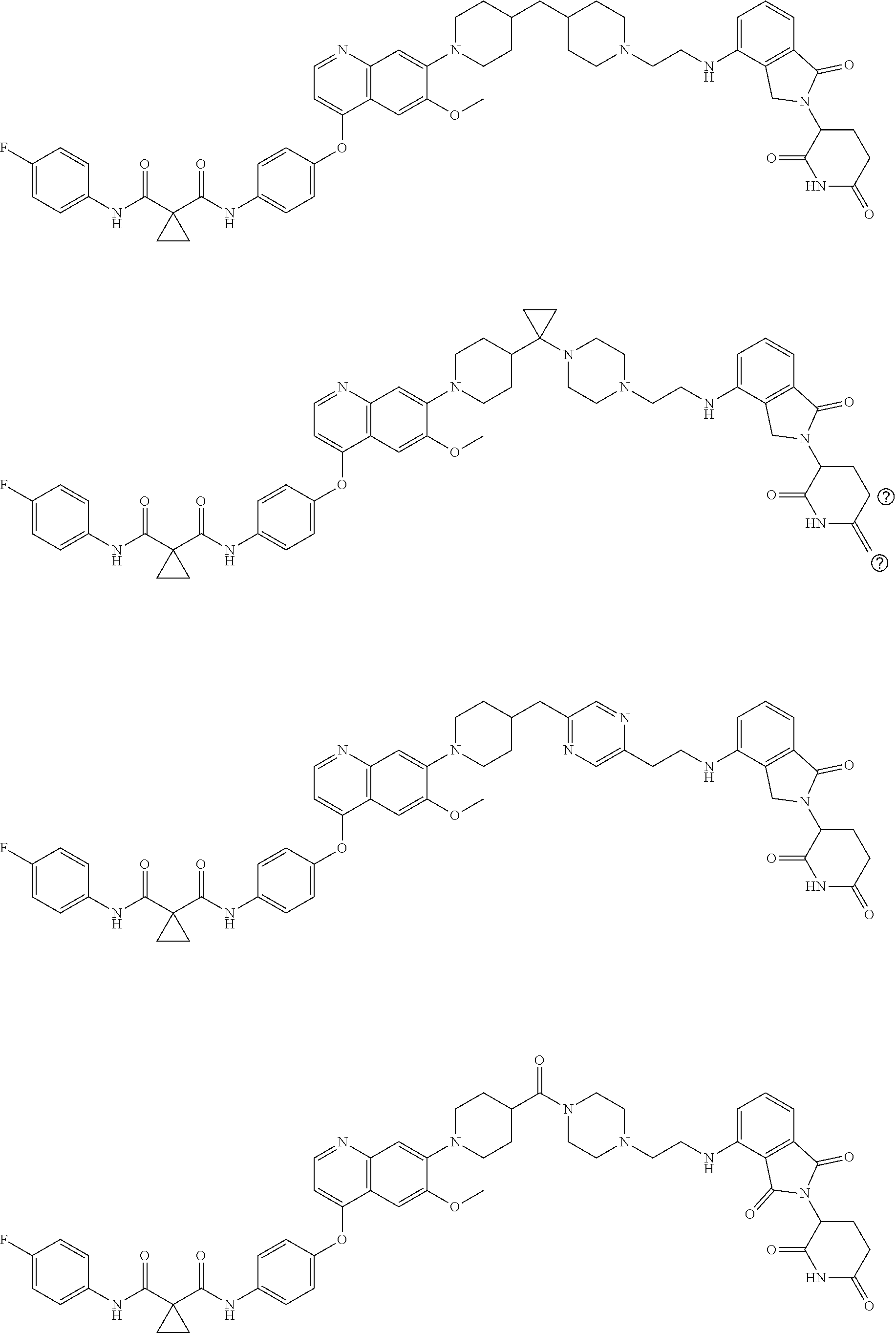 [SMILES: COc1cc2c(Oc3ccc(NC(=O)C4(C(=O)Nc5ccc(F)cc5)CC4)cc3)ccnc2cc1N1CCC(CC2CCN(CCNc3cccc4c3CN(C3CCC(=O)NC3=O)C4=O)CC2)CC1 ; C=C1CCC(N2Cc3c(NCCN4CCN(C5(C6CCN(c7cc8nccc(Oc9ccc(NC(=O)C%10(C(=O)Nc%11ccc(F)cc%11)CC%10)cc9)c8cc7OC)CC6)CC5)CC4)cccc3C2=O)C(=O)N1 ; COc1cc2c(Oc3ccc(NC(=O)C4(C(=O)Nc5ccc(F)cc5)CC4)cc3)ccnc2cc1N1CCC(Cc2cnc(CCNc3cccc4c3CN(C3CCC(=O)NC3=O)C4=O)cn2)CC1 ; COc1cc2c(Oc3ccc(NC(=O)C4(C(=O)Nc5ccc(F)cc5)CC4)cc3)ccnc2cc1N1CCC(C(=O)N2CCN(CCNc3cccc4c3C(=O)N(C3CCC(=O)NC3=O)C4=O)CC2)CC1]

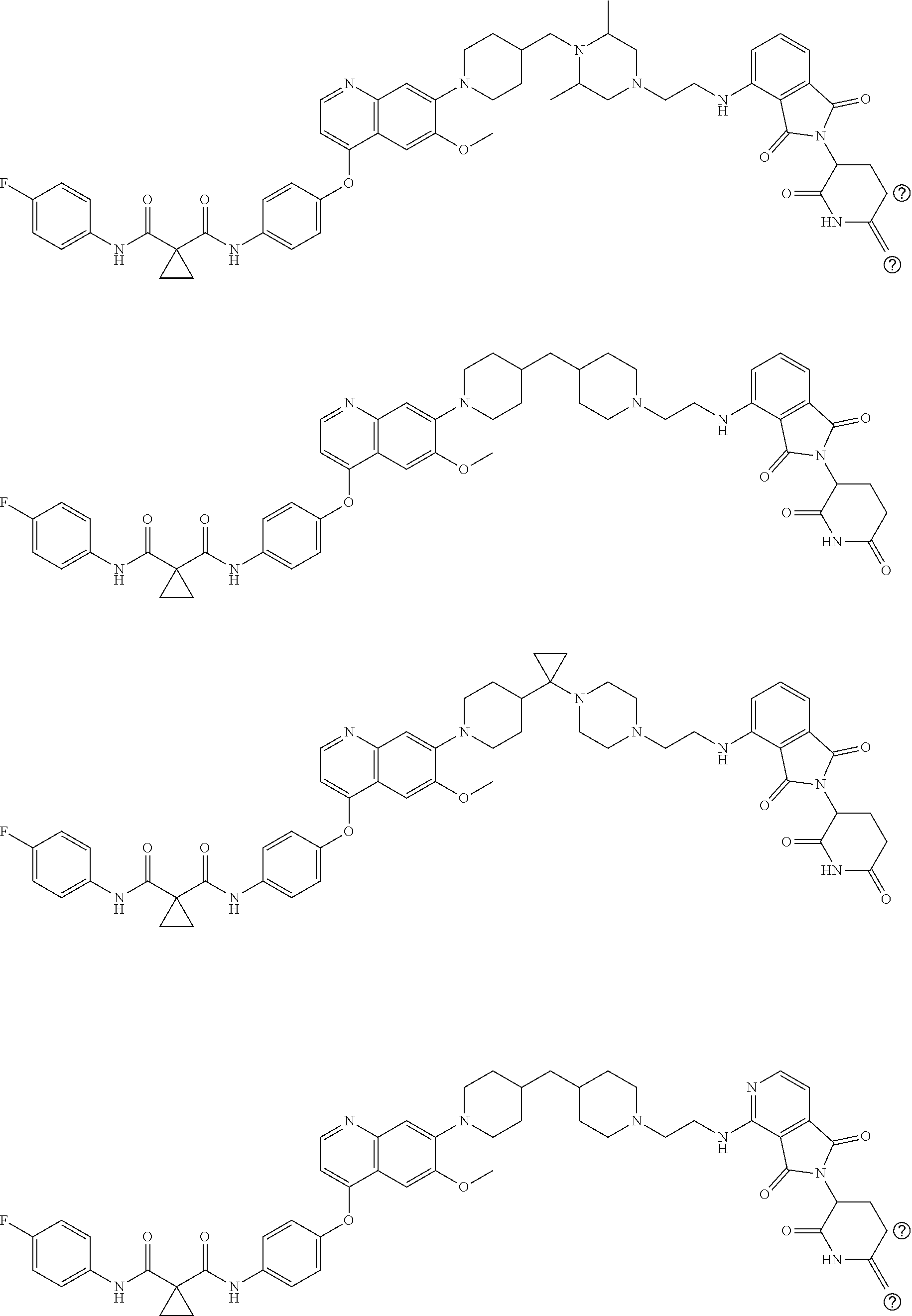

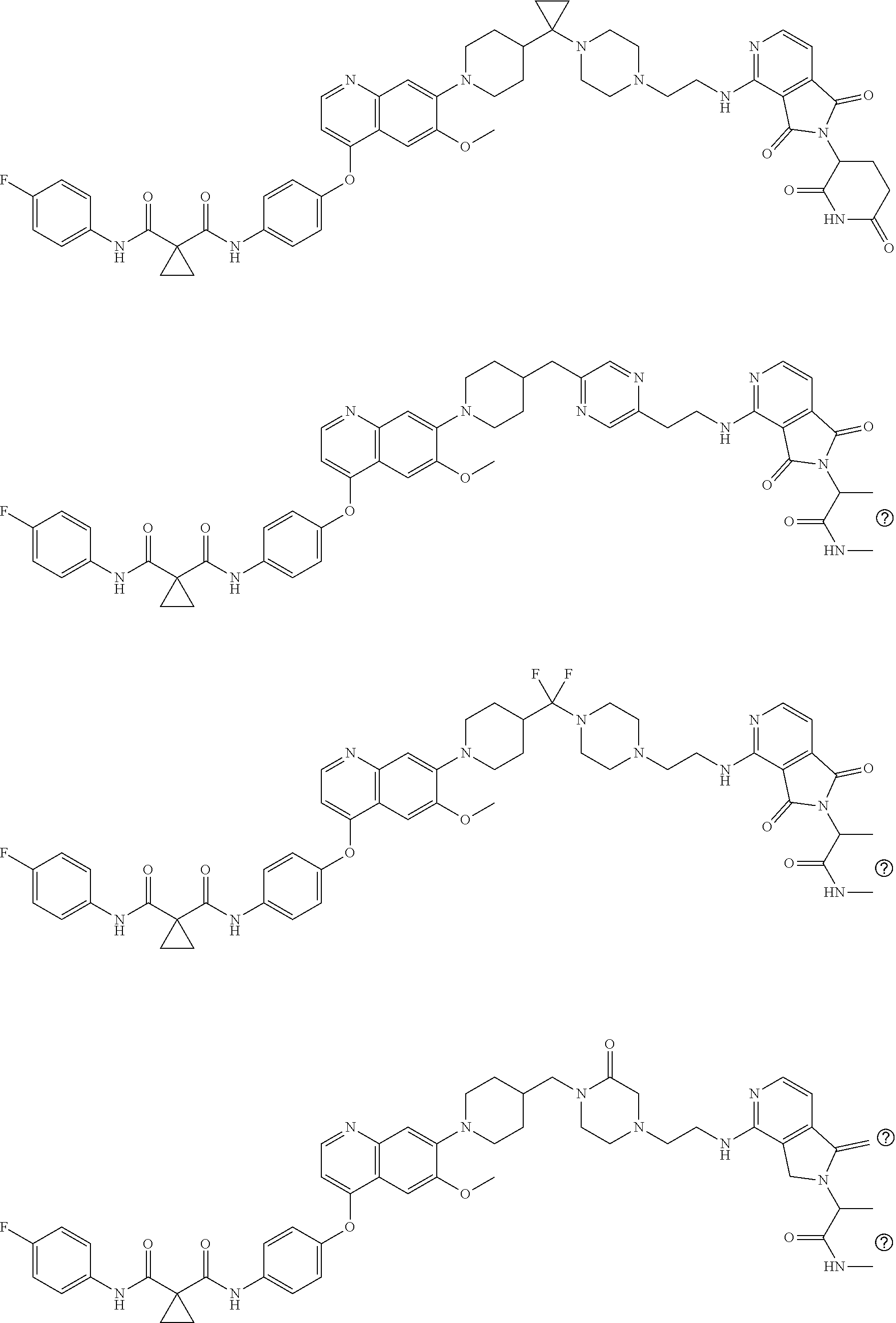 [SMILES: COc1cc2c(Oc3ccc(NC(=O)C4(C(=O)Nc5ccc(F)cc5)CC4)cc3)ccnc2cc1N1CCC(C2(N3CCN(CCNc4nccc5c4C(=O)N(C4CCC(=O)NC4=O)C5=O)CC3)CC2)CC1 ; CNC(=O)C(C)N1C(=O)c2ccnc(NCCc3cnc(CC4CCN(c5cc6nccc(Oc7ccc(NC(=O)C8(C(=O)Nc9ccc(F)cc9)CC8)cc7)c6cc5OC)CC4)cn3)c2C1=O ; CNC(=O)C(C)N1C(=O)c2ccnc(NCCN3CCN(C(F)(F)C4CCN(c5cc6nccc(Oc7ccc(NC(=O)C8(C(=O)Nc9ccc(F)cc9)CC8)cc7)c6cc5OC)CC4)CC3)c2C1=O ; C=C1c2ccnc(NCCN3CCN(CC4CCN(c5cc6nccc(Oc7ccc(NC(=O)C8(C(=O)Nc9ccc(F)cc9)CC8)cc7)c6cc5OC)CC4)C(=O)C3)c2CN1C(C)C(=O)NC]

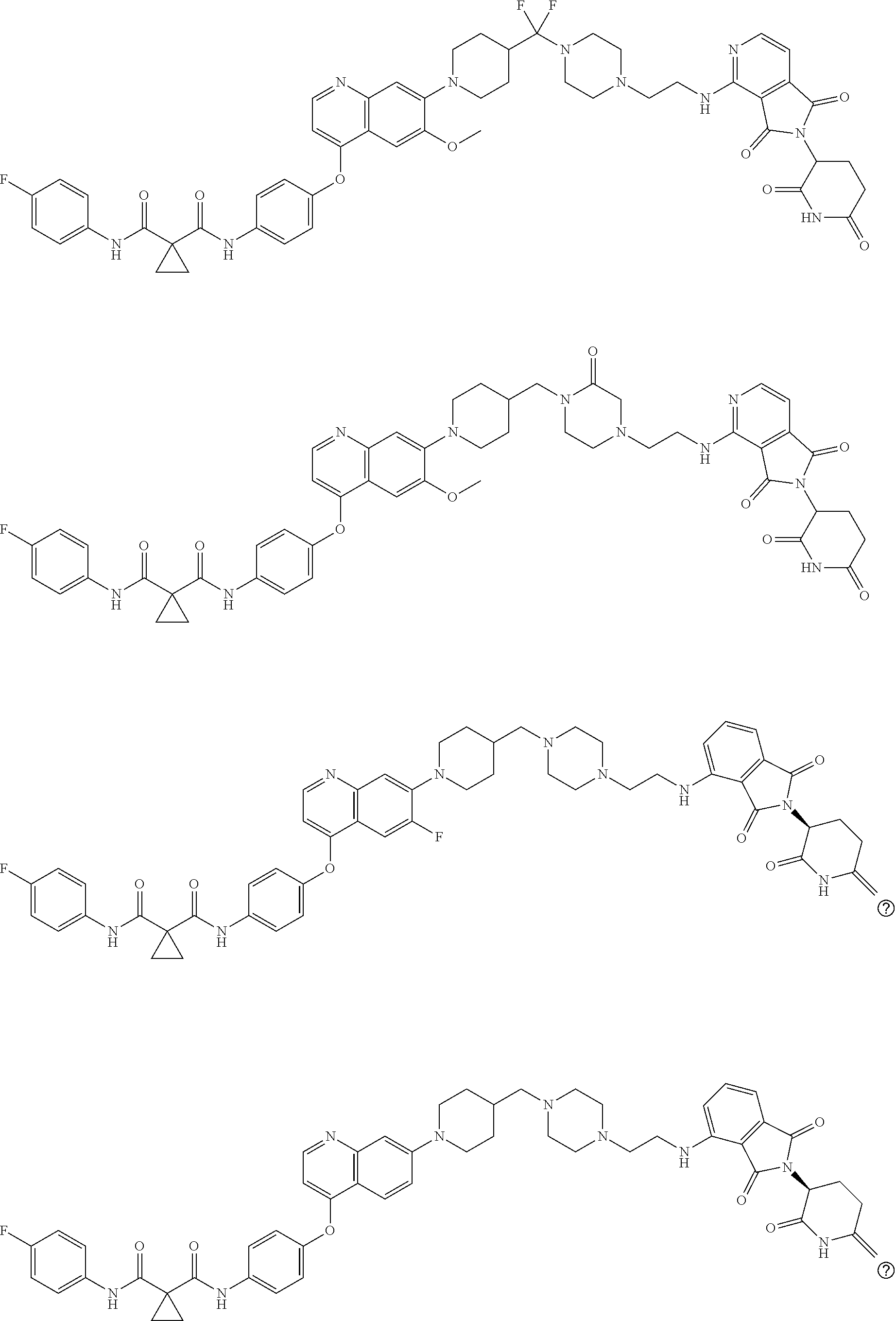

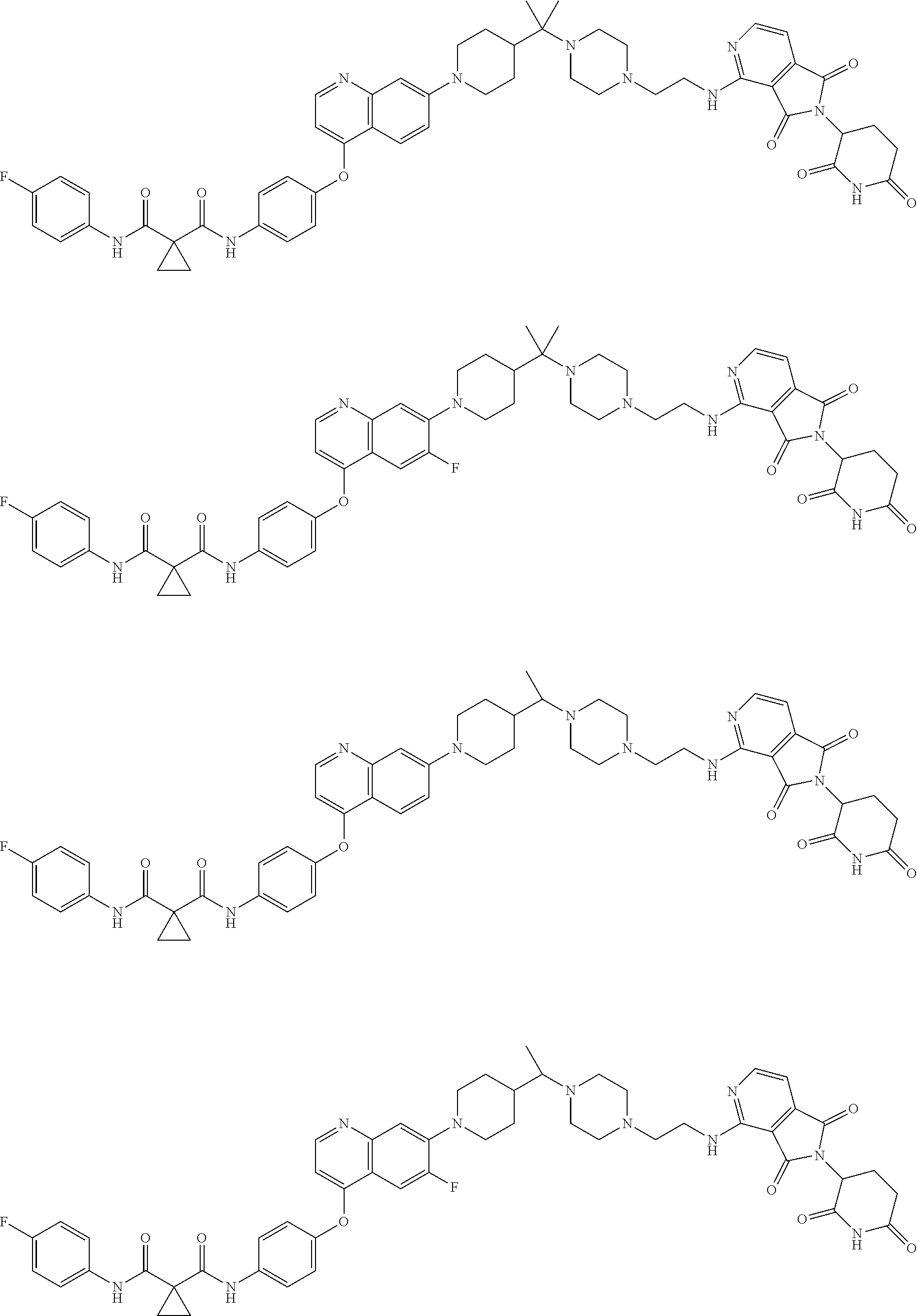 [SMILES: CC(C)(C1CCN(c2ccc3c(Oc4ccc(NC(=O)C5(C(=O)Nc6ccc(F)cc6)CC5)cc4)ccnc3c2)CC1)N1CCN(CCNc2nccc3c2C(=O)N(C2CCC(=O)NC2=O)C3=O)CC1 ; CC(C)(C1CCN(c2cc3nccc(Oc4ccc(NC(=O)C5(C(=O)Nc6ccc(F)cc6)CC5)cc4)c3cc2F)CC1)N1CCN(CCNc2nccc3c2C(=O)N(C2CCC(=O)NC2=O)C3=O)CC1 ; CC(C1CCN(c2ccc3c(Oc4ccc(NC(=O)C5(C(=O)Nc6ccc(F)cc6)CC5)cc4)ccnc3c2)CC1)N1CCN(CCNc2nccc3c2C(=O)N(C2CCC(=O)NC2=O)C3=O)CC1 ; CC(C1CCN(c2cc3nccc(Oc4ccc(NC(=O)C5(C(=O)Nc6ccc(F)cc6)CC5)cc4)c3cc2F)CC1)N1CCN(CCNc2nccc3c2C(=O)N(C2CCC(=O)NC2=O)C3=O)CC1]

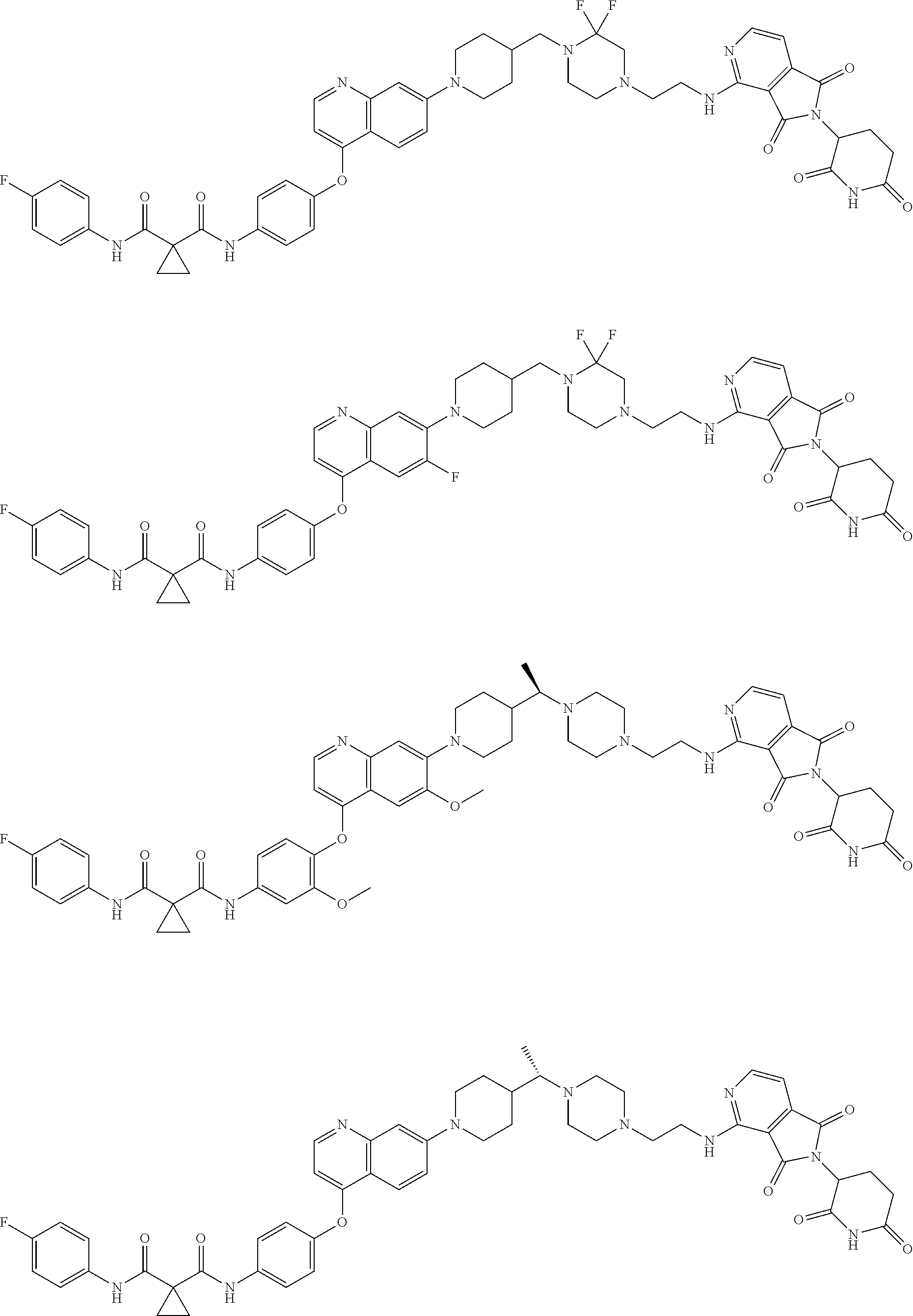

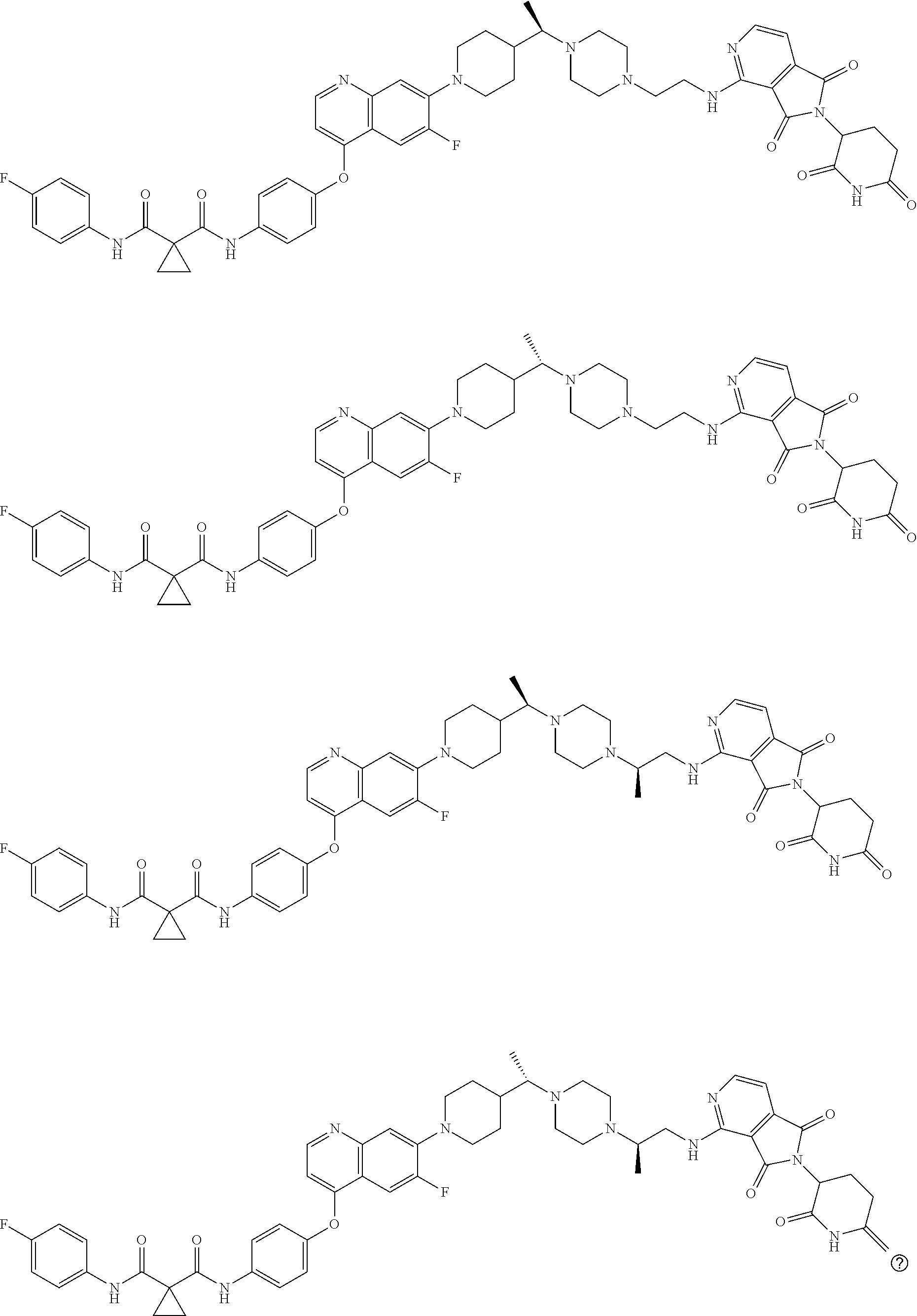

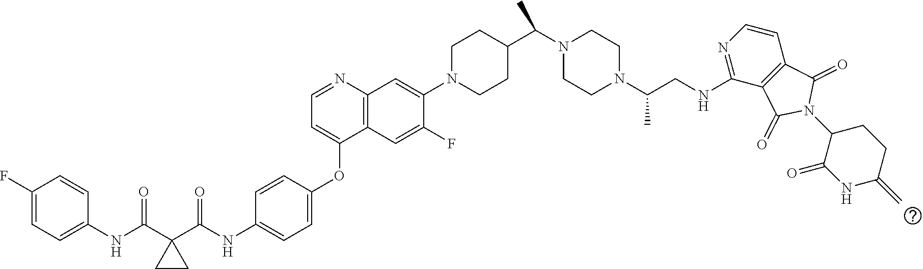 [SMILES: C=C1CCC(N2C(=O)c3ccnc(NC[C@H](C)N4CCN([C@H](C)C5CCN(c6cc7nccc(Oc8ccc(NC(=O)C9(C(=O)Nc%10ccc(F)cc%10)CC9)cc8)c7cc6F)CC5)CC4)c3C2=O)C(=O)N1]

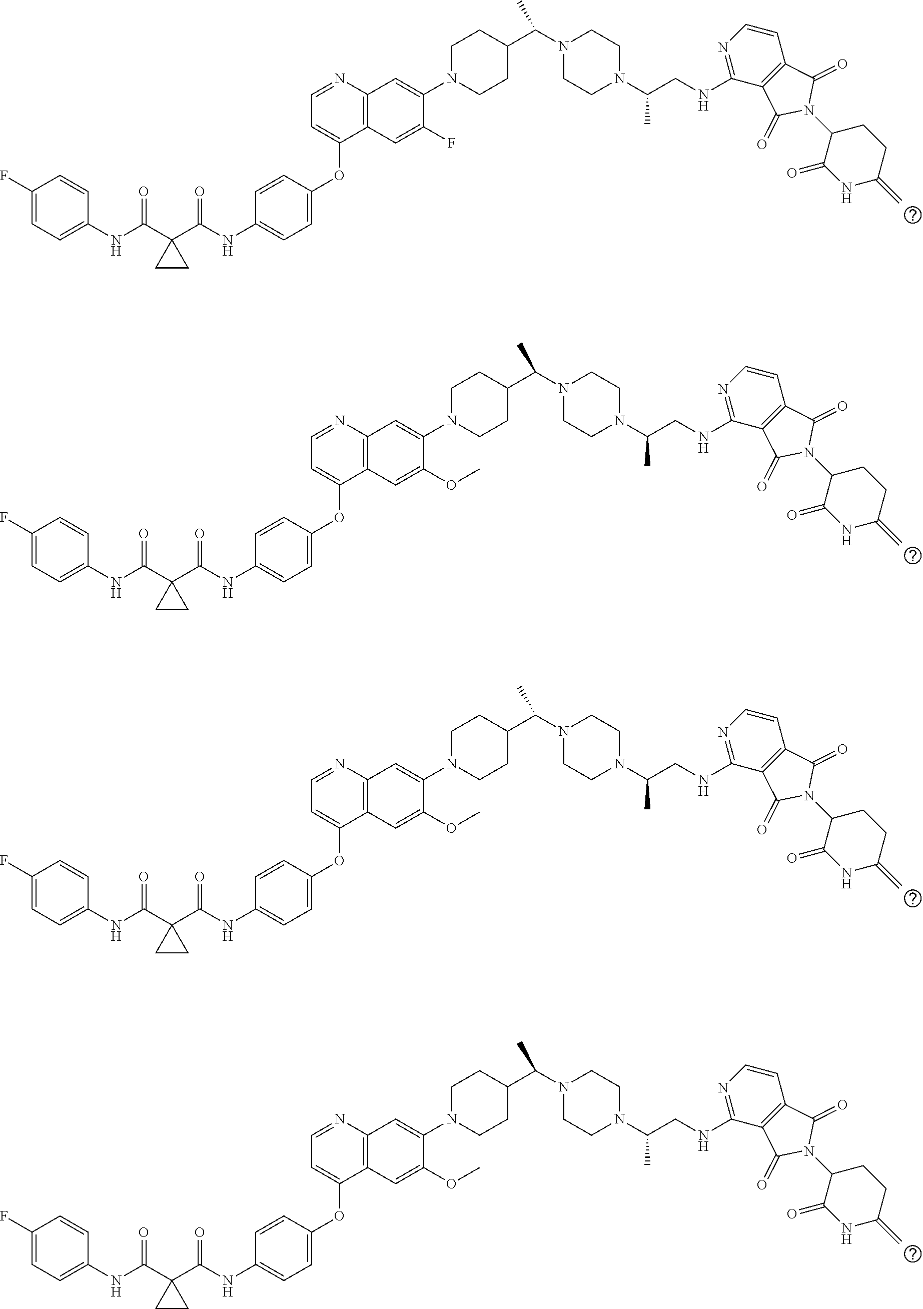

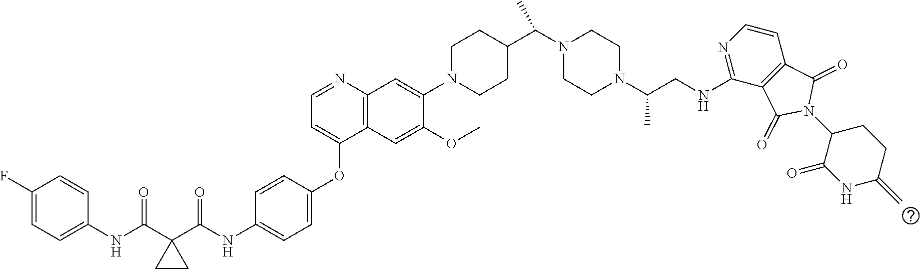 [SMILES: C=C1CCC(N2C(=O)c3ccnc(NC[C@H](C)N4CCN([C@@H](C)C5CCN(c6cc7nccc(Oc8ccc(NC(=O)C9(C(=O)Nc%10ccc(F)cc%10)CC9)cc8)c7cc6OC)CC5)CC4)c3C2=O)C(=O)N1]

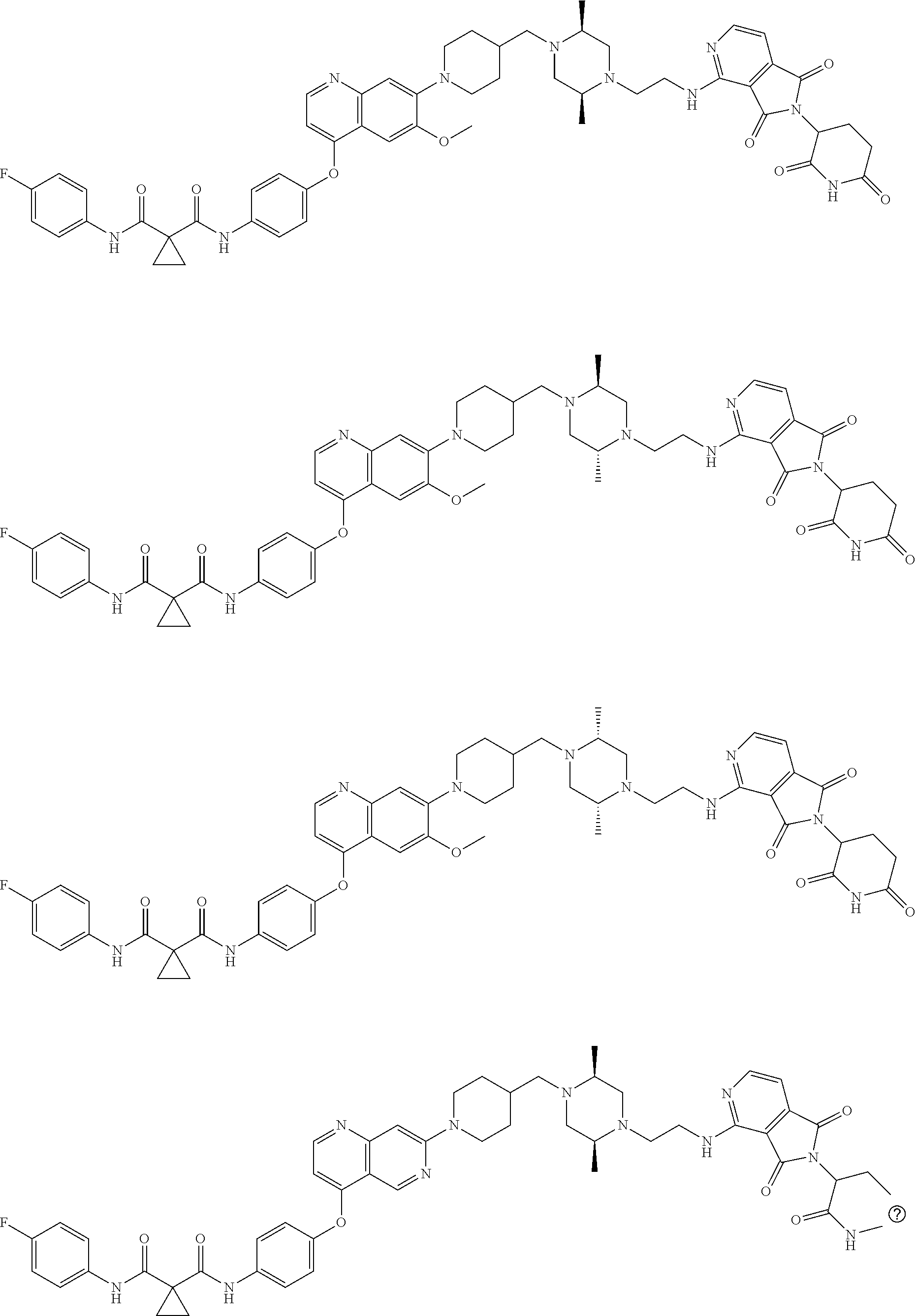

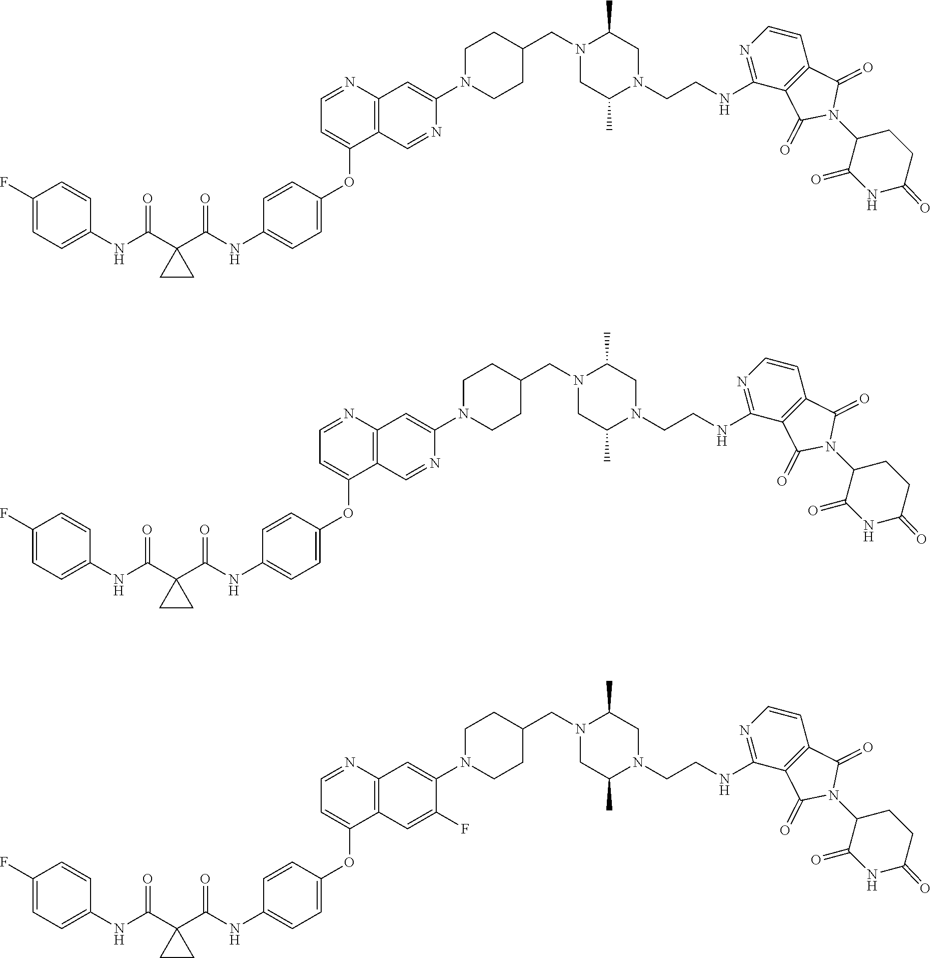 [SMILES: C[C@@H]1CN(CC2CCN(c3cc4nccc(Oc5ccc(NC(=O)C6(C(=O)Nc7ccc(F)cc7)CC6)cc5)c4cn3)CC2)[C@@H](C)CN1CCNc1nccc2c1C(=O)N(C1CCC(=O)NC1=O)C2=O ; C[C@@H]1CN(CC2CCN(c3cc4nccc(Oc5ccc(NC(=O)C6(C(=O)Nc7ccc(F)cc7)CC6)cc5)c4cn3)CC2)[C@H](C)CN1CCNc1nccc2c1C(=O)N(C1CCC(=O)NC1=O)C2=O ; C[C@H]1CN(CC2CCN(c3cc4nccc(Oc5ccc(NC(=O)C6(C(=O)Nc7ccc(F)cc7)CC6)cc5)c4cc3F)CC2)[C@@H](C)CN1CCNc1nccc2c1C(=O)N(C1CCC(=O)NC1=O)C2=O]

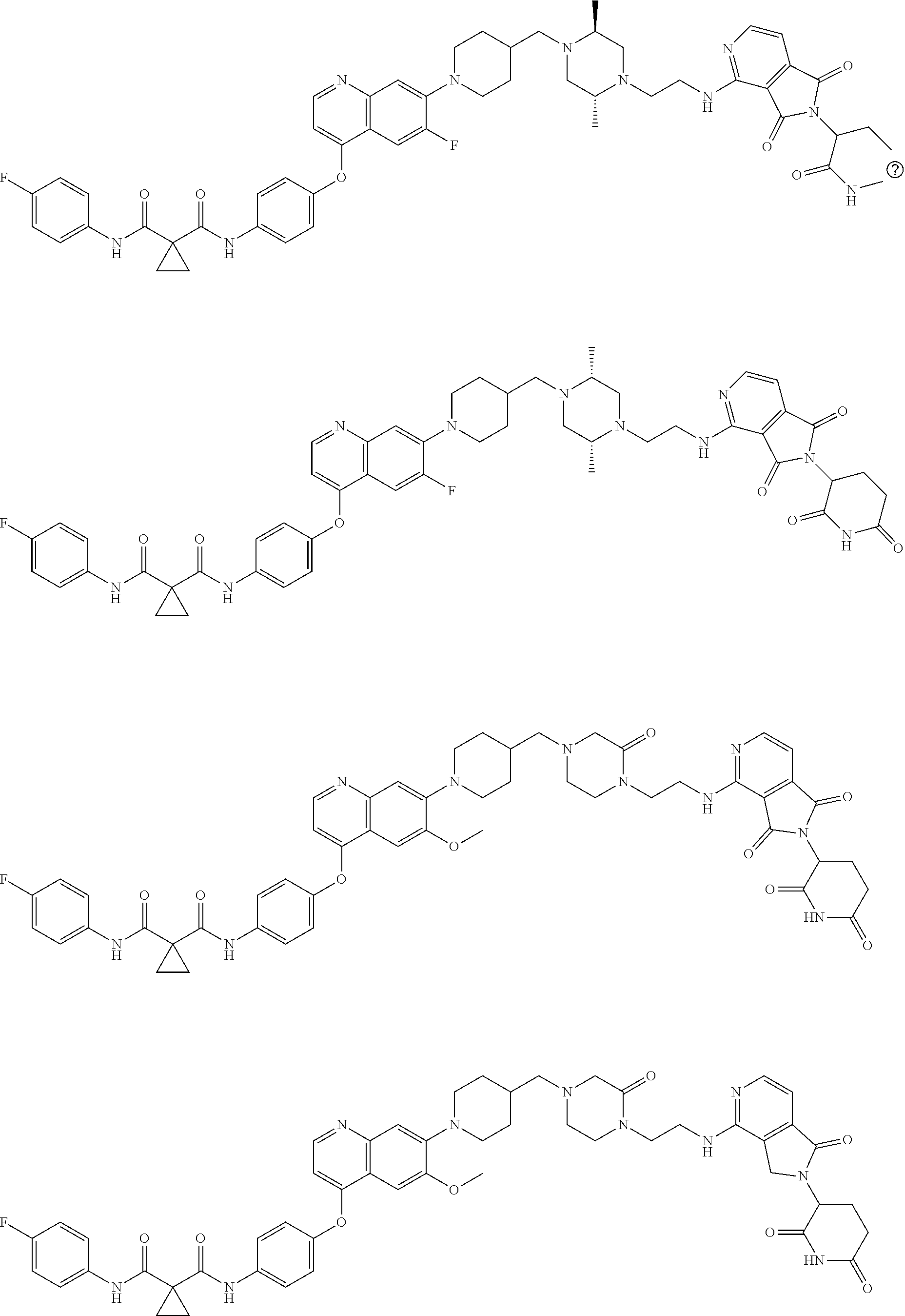

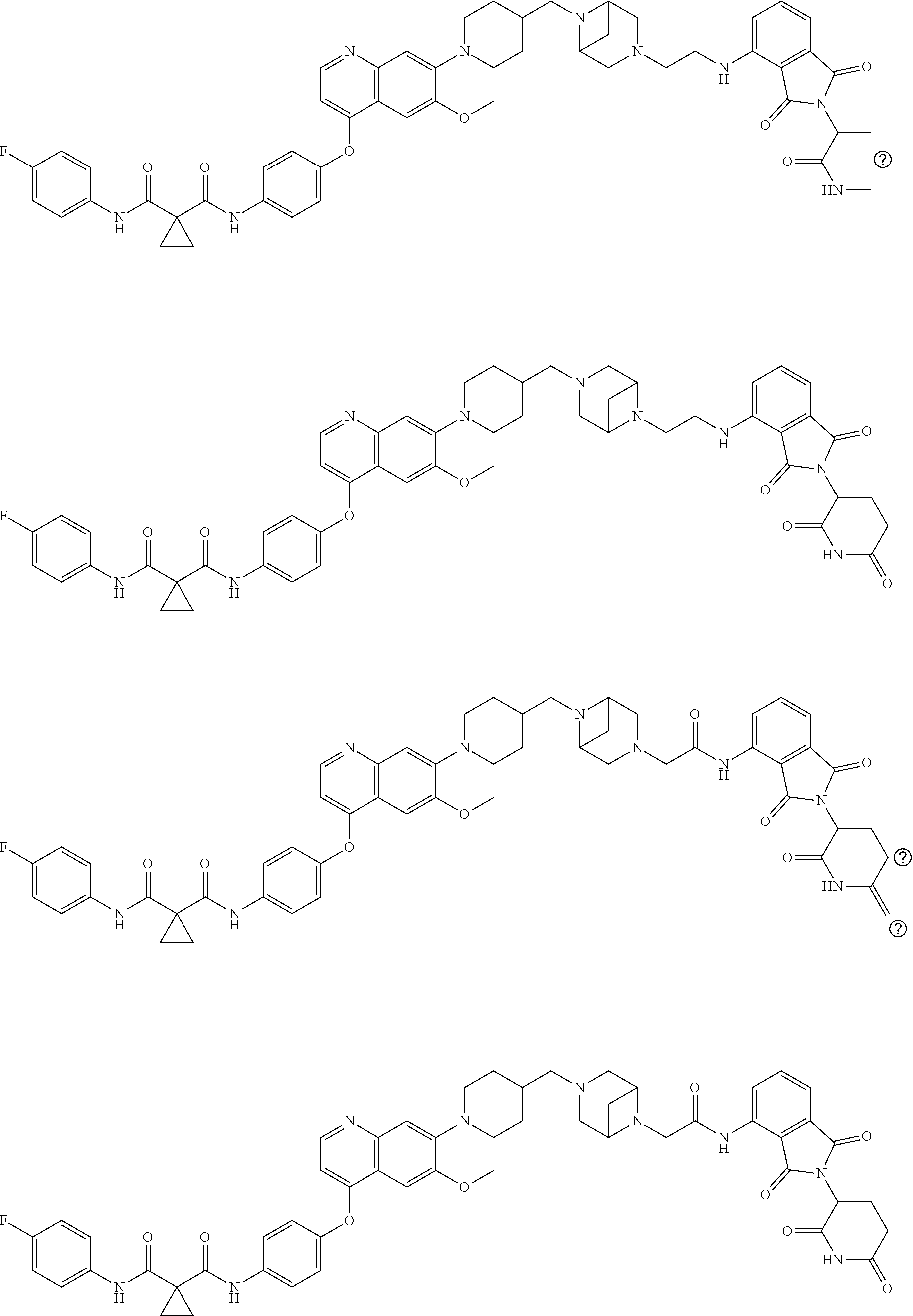 [SMILES: CNC(=O)C(C)N1C(=O)c2cccc(NCCN3CC4CC(C3)N4CC3CCN(c4cc5nccc(Oc6ccc(NC(=O)C7(C(=O)Nc8ccc(F)cc8)CC7)cc6)c5cc4OC)CC3)c2C1=O ; COc1cc2c(Oc3ccc(NC(=O)C4(C(=O)Nc5ccc(F)cc5)CC4)cc3)ccnc2cc1N1CCC(CN2CC3CC(C2)N3CCNc2cccc3c2C(=O)N(C2CCC(=O)NC2=O)C3=O)CC1 ; C=C1CCC(N2C(=O)c3cccc(NC(=O)CN4CC5CC(C4)N5CC4CCN(c5cc6nccc(Oc7ccc(NC(=O)C8(C(=O)Nc9ccc(F)cc9)CC8)cc7)c6cc5OC)CC4)c3C2=O)C(=O)N1 ; COc1cc2c(Oc3ccc(NC(=O)C4(C(=O)Nc5ccc(F)cc5)CC4)cc3)ccnc2cc1N1CCC(CN2CC3CC(C2)N3CC(=O)Nc2cccc3c2C(=O)N(C2CCC(=O)NC2=O)C3=O)CC1]

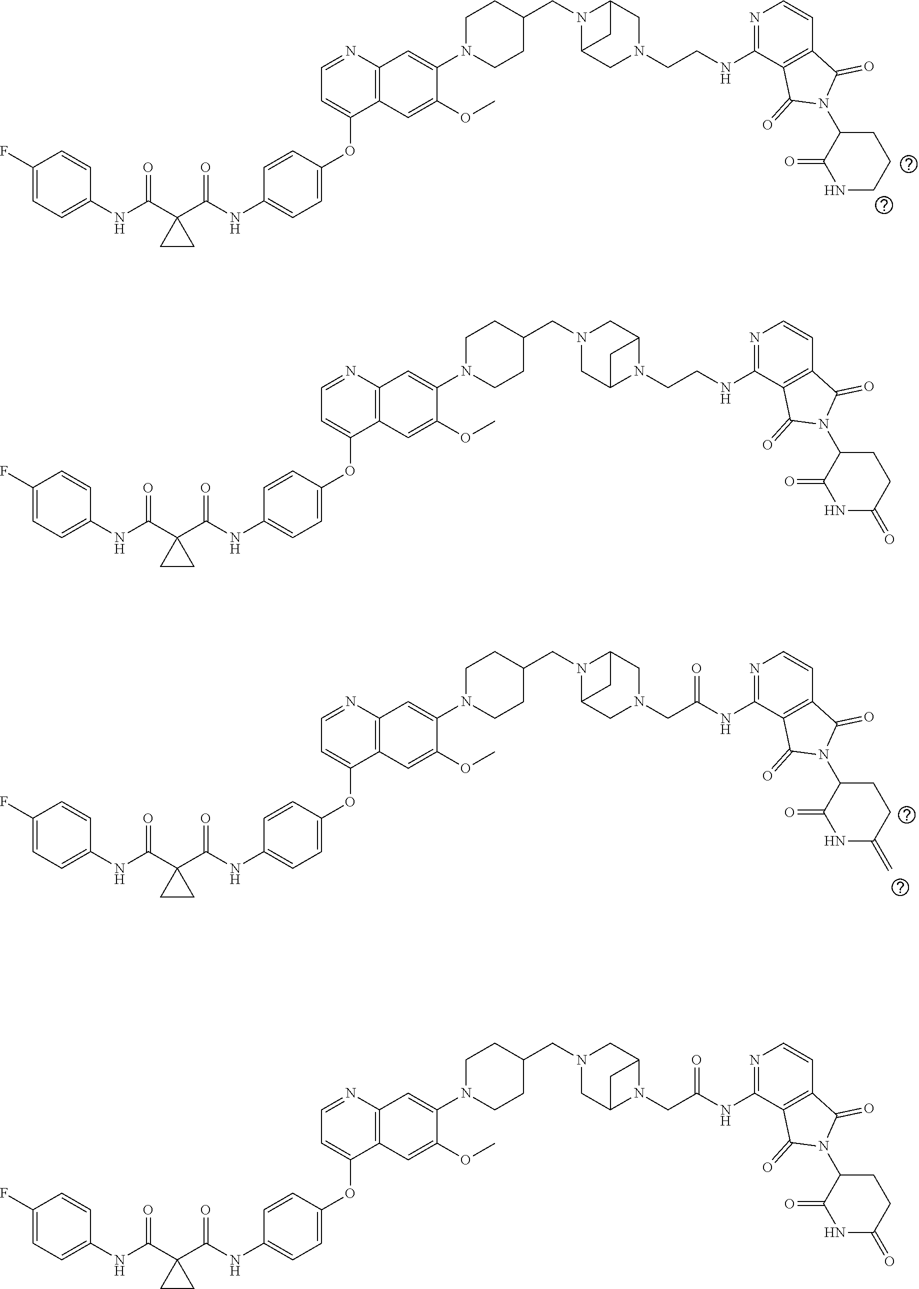 [SMILES: COc1cc2c(Oc3ccc(NC(=O)C4(C(=O)Nc5ccc(F)cc5)CC4)cc3)ccnc2cc1N1CCC(CN2C3CC2CN(CCNc2nccc4c2C(=O)N(C2CCCNC2=O)C4=O)C3)CC1 ; COc1cc2c(Oc3ccc(NC(=O)C4(C(=O)Nc5ccc(F)cc5)CC4)cc3)ccnc2cc1N1CCC(CN2CC3CC(C2)N3CCNc2nccc3c2C(=O)N(C2CCC(=O)NC2=O)C3=O)CC1 ; C=C1CCC(N2C(=O)c3ccnc(NC(=O)CN4CC5CC(C4)N5CC4CCN(c5cc6nccc(Oc7ccc(NC(=O)C8(C(=O)Nc9ccc(F)cc9)CC8)cc7)c6cc5OC)CC4)c3C2=O)C(=O)N1 ; COc1cc2c(Oc3ccc(NC(=O)C4(C(=O)Nc5ccc(F)cc5)CC4)cc3)ccnc2cc1N1CCC(CN2CC3CC(C2)N3CC(=O)Nc2nccc3c2C(=O)N(C2CCC(=O)NC2=O)C3=O)CC1]

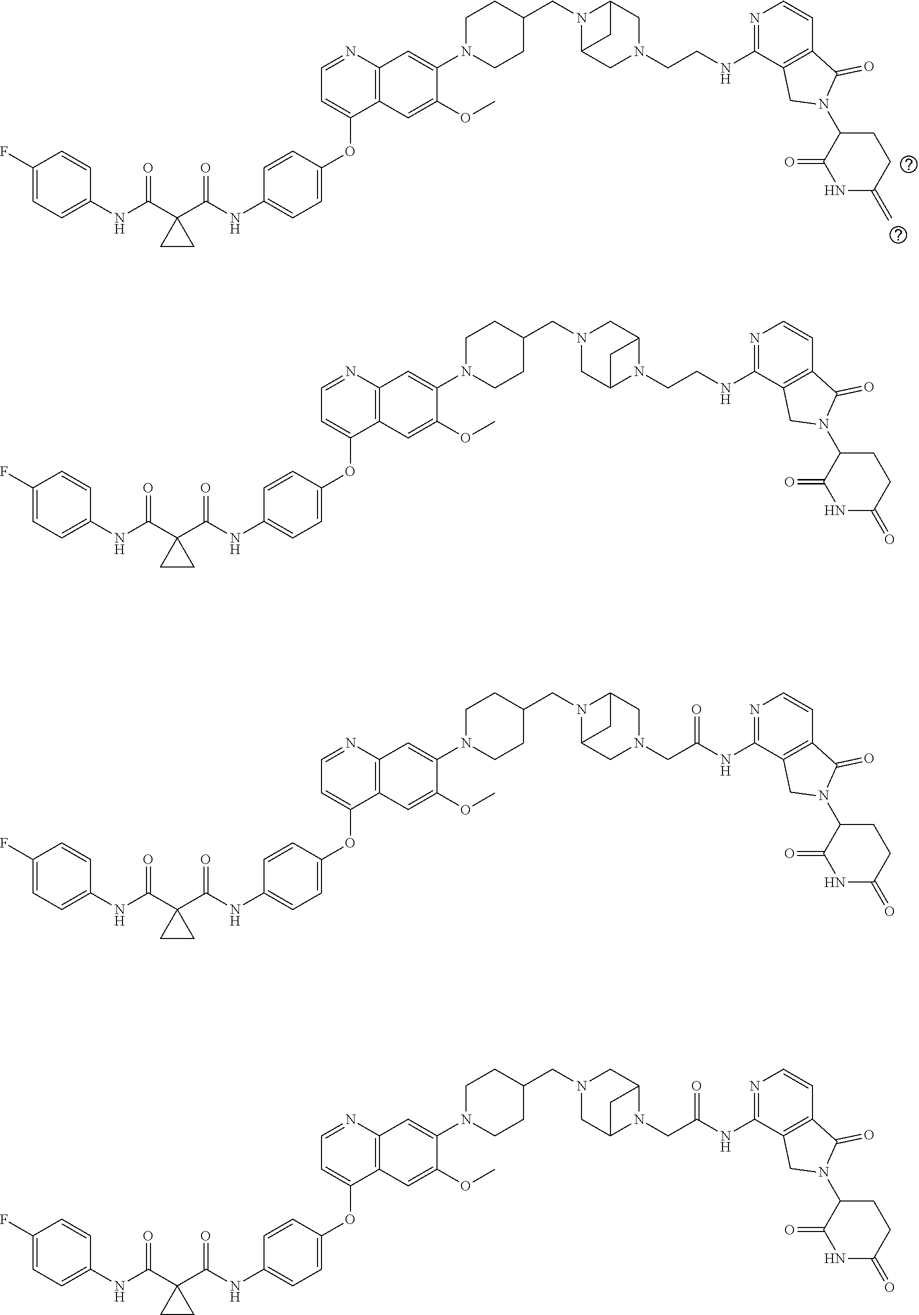 [SMILES: C=C1CCC(N2Cc3c(ccnc3NCCN3CC4CC(C3)N4CC3CCN(c4cc5nccc(Oc6ccc(NC(=O)C7(C(=O)Nc8ccc(F)cc8)CC7)cc6)c5cc4OC)CC3)C2=O)C(=O)N1 ; COc1cc2c(Oc3ccc(NC(=O)C4(C(=O)Nc5ccc(F)cc5)CC4)cc3)ccnc2cc1N1CCC(CN2CC3CC(C2)N3CCNc2nccc3c2CN(C2CCC(=O)NC2=O)C3=O)CC1 ; COc1cc2c(Oc3ccc(NC(=O)C4(C(=O)Nc5ccc(F)cc5)CC4)cc3)ccnc2cc1N1CCC(CN2C3CC2CN(CC(=O)Nc2nccc4c2CN(C2CCC(=O)NC2=O)C4=O)C3)CC1 ; COc1cc2c(Oc3ccc(NC(=O)C4(C(=O)Nc5ccc(F)cc5)CC4)cc3)ccnc2cc1N1CCC(CN2CC3CC(C2)N3CC(=O)Nc2nccc3c2CN(C2CCC(=O)NC2=O)C3=O)CC1]

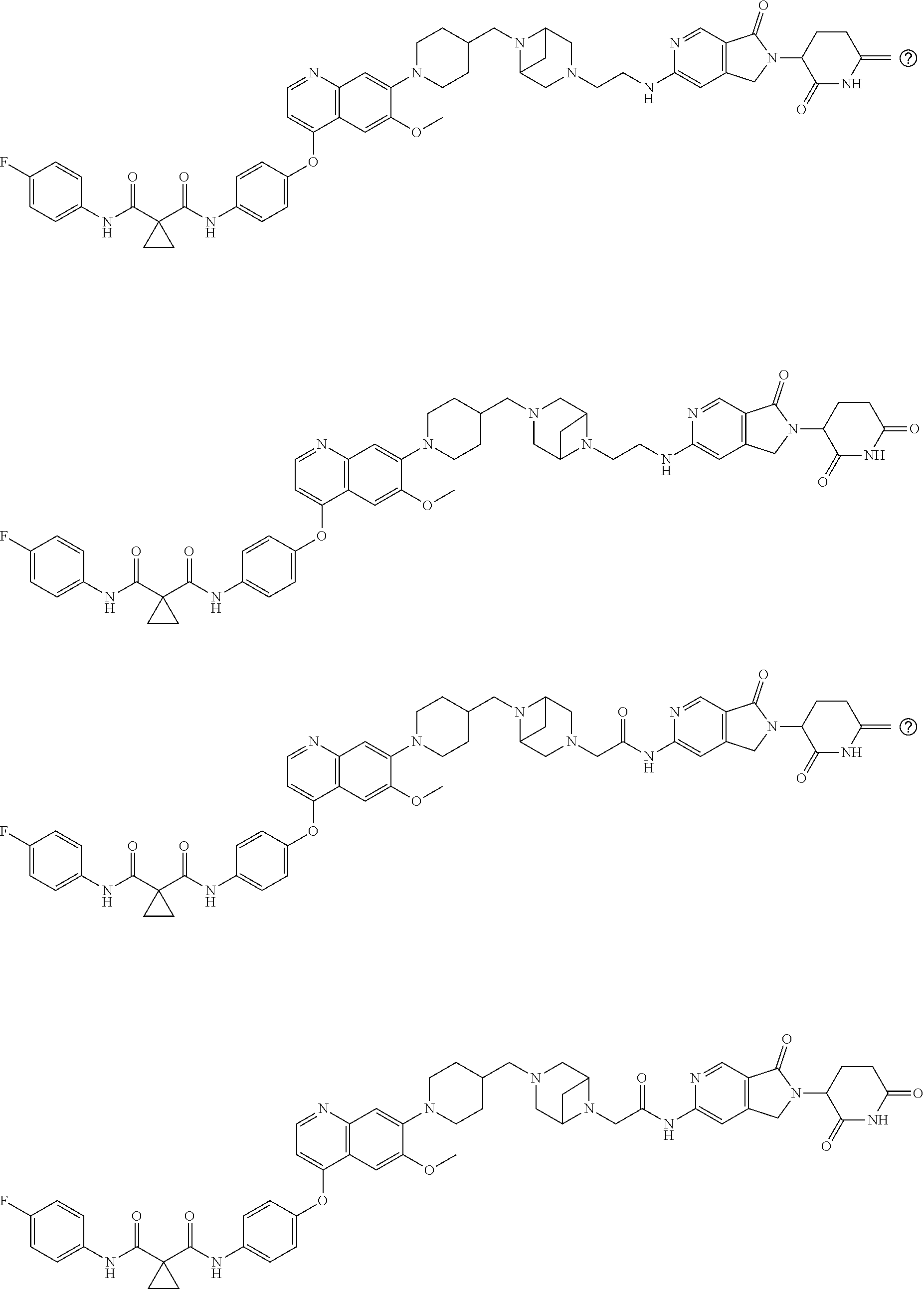 [SMILES: C=C1CCC(N2Cc3cc(NCCN4CC5CC(C4)N5CC4CCN(c5cc6nccc(Oc7ccc(NC(=O)C8(C(=O)Nc9ccc(F)cc9)CC8)cc7)c6cc5OC)CC4)ncc3C2=O)C(=O)N1 ; COc1cc2c(Oc3ccc(NC(=O)C4(C(=O)Nc5ccc(F)cc5)CC4)cc3)ccnc2cc1N1CCC(CN2CC3CC(C2)N3CCNc2cc3c(cn2)C(=O)N(C2CCC(=O)NC2=O)C3)CC1 ; C=C1CCC(N2Cc3cc(NC(=O)CN4CC5CC(C4)N5CC4CCN(c5cc6nccc(Oc7ccc(NC(=O)C8(C(=O)Nc9ccc(F)cc9)CC8)cc7)c6cc5OC)CC4)ncc3C2=O)C(=O)N1 ; COc1cc2c(Oc3ccc(NC(=O)C4(C(=O)Nc5ccc(F)cc5)CC4)cc3)ccnc2cc1N1CCC(CN2CC3CC(C2)N3CC(=O)Nc2cc3c(cn2)C(=O)N(C2CCC(=O)NC2=O)C3)CC1]

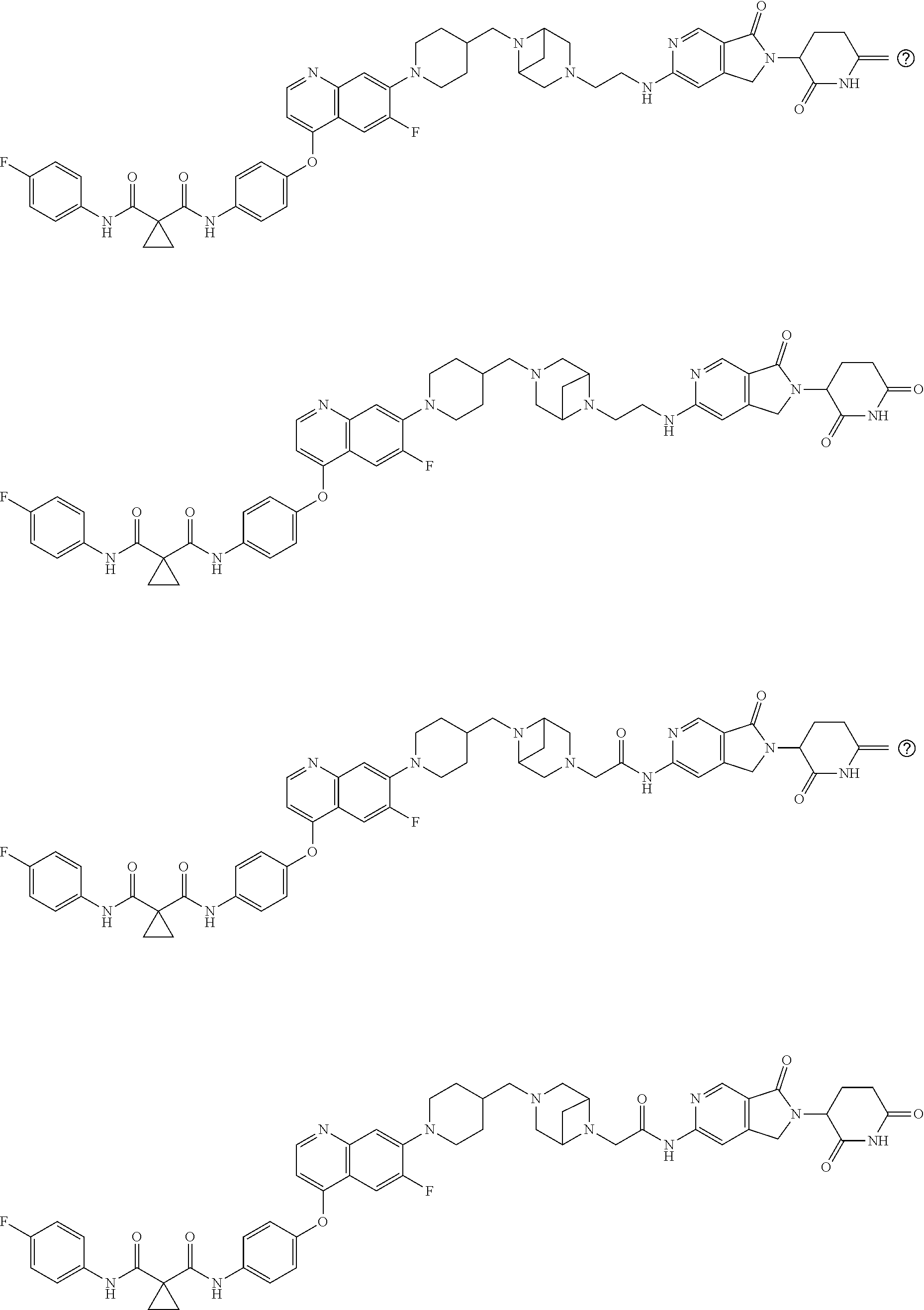 [SMILES: C=C1CCC(N2Cc3cc(NCCN4CC5CC(C4)N5CC4CCN(c5cc6nccc(Oc7ccc(NC(=O)C8(C(=O)Nc9ccc(F)cc9)CC8)cc7)c6cc5F)CC4)ncc3C2=O)C(=O)N1 ; O=C1CCC(N2Cc3cc(NCCN4C5CC4CN(CC4CCN(c6cc7nccc(Oc8ccc(NC(=O)C9(C(=O)Nc%10ccc(F)cc%10)CC9)cc8)c7cc6F)CC4)C5)ncc3C2=O)C(=O)N1 ; C=C1CCC(N2Cc3cc(NC(=O)CN4CC5CC(C4)N5CC4CCN(c5cc6nccc(Oc7ccc(NC(=O)C8(C(=O)Nc9ccc(F)cc9)CC8)cc7)c6cc5F)CC4)ncc3C2=O)C(=O)N1 ; O=C1CCC(N2Cc3cc(NC(=O)CN4C5CC4CN(CC4CCN(c6cc7nccc(Oc8ccc(NC(=O)C9(C(=O)Nc%10ccc(F)cc%10)CC9)cc8)c7cc6F)CC4)C5)ncc3C2=O)C(=O)N1]

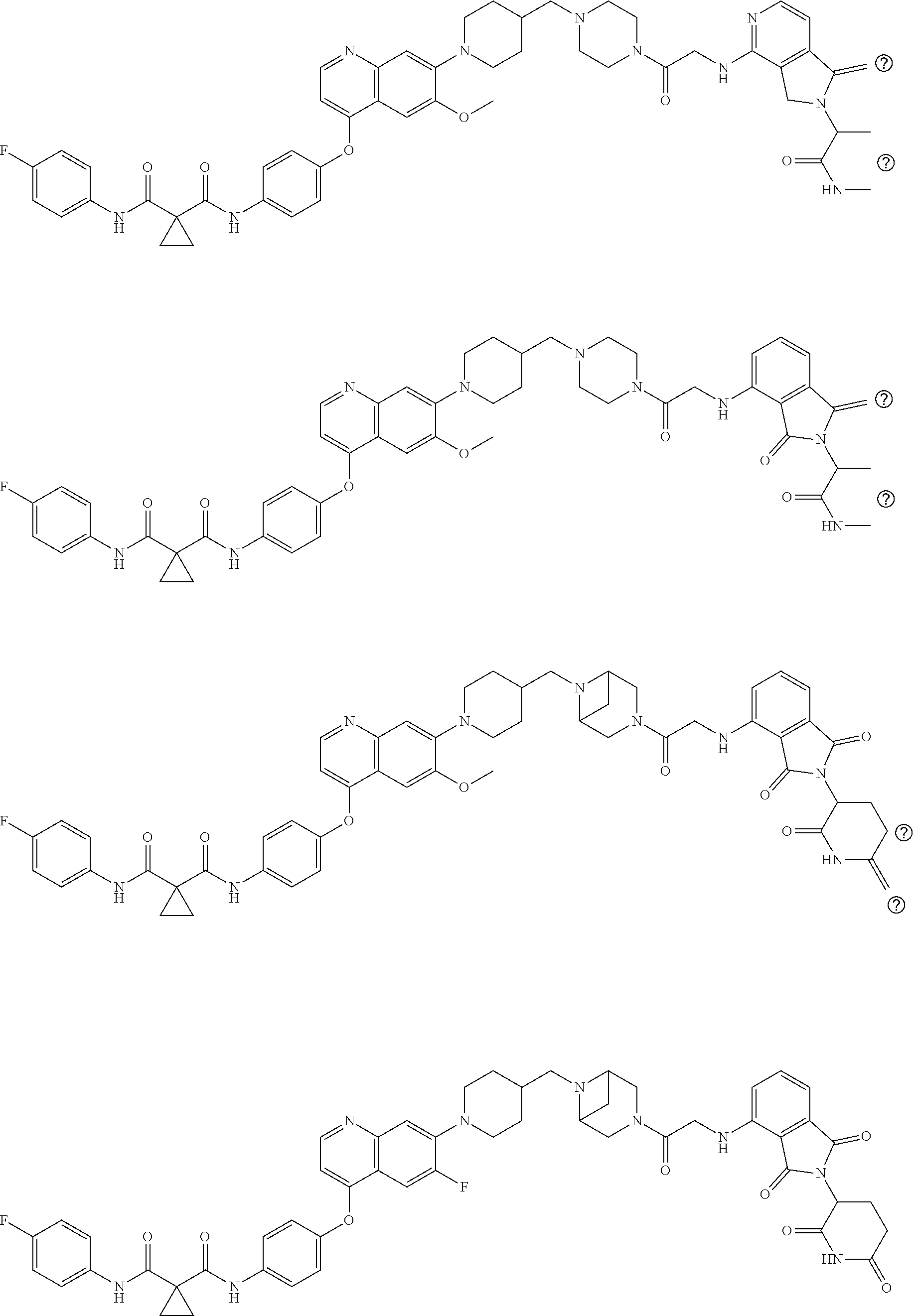 [SMILES: C=C1c2ccnc(NCC(=O)N3CCN(CC4CCN(c5cc6nccc(Oc7ccc(NC(=O)C8(C(=O)Nc9ccc(F)cc9)CC8)cc7)c6cc5OC)CC4)CC3)c2CN1C(C)C(=O)NC ; C=C1c2cccc(NCC(=O)N3CCN(CC4CCN(c5cc6nccc(Oc7ccc(NC(=O)C8(C(=O)Nc9ccc(F)cc9)CC8)cc7)c6cc5OC)CC4)CC3)c2C(=O)N1C(C)C(=O)NC ; C=C1CCC(N2C(=O)c3cccc(NCC(=O)N4CC5CC(C4)N5CC4CCN(c5cc6nccc(Oc7ccc(NC(=O)C8(C(=O)Nc9ccc(F)cc9)CC8)cc7)c6cc5OC)CC4)c3C2=O)C(=O)N1 ; O=C1CCC(N2C(=O)c3cccc(NCC(=O)N4CC5CC(C4)N5CC4CCN(c5cc6nccc(Oc7ccc(NC(=O)C8(C(=O)Nc9ccc(F)cc9)CC8)cc7)c6cc5F)CC4)c3C2=O)C(=O)N1]

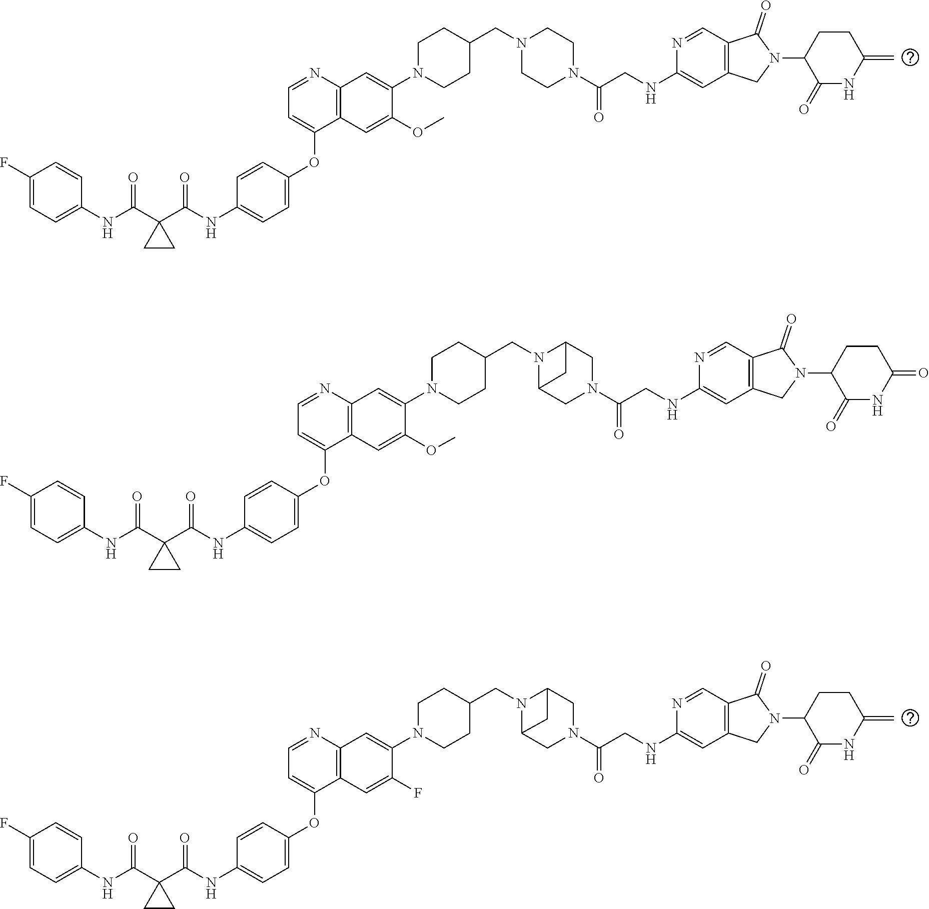 [SMILES: C=C1CCC(N2Cc3cc(NCC(=O)N4CCN(CC5CCN(c6cc7nccc(Oc8ccc(NC(=O)C9(C(=O)Nc%10ccc(F)cc%10)CC9)cc8)c7cc6OC)CC5)CC4)ncc3C2=O)C(=O)N1 ; COc1cc2c(Oc3ccc(NC(=O)C4(C(=O)Nc5ccc(F)cc5)CC4)cc3)ccnc2cc1N1CCC(CN2C3CC2CN(C(=O)CNc2cc4c(cn2)C(=O)N(C2CCC(=O)NC2=O)C4)C3)CC1 ; C=C1CCC(N2Cc3cc(NCC(=O)N4CC5CC(C4)N5CC4CCN(c5cc6nccc(Oc7ccc(NC(=O)C8(C(=O)Nc9ccc(F)cc9)CC8)cc7)c6cc5F)CC4)ncc3C2=O)C(=O)N1]

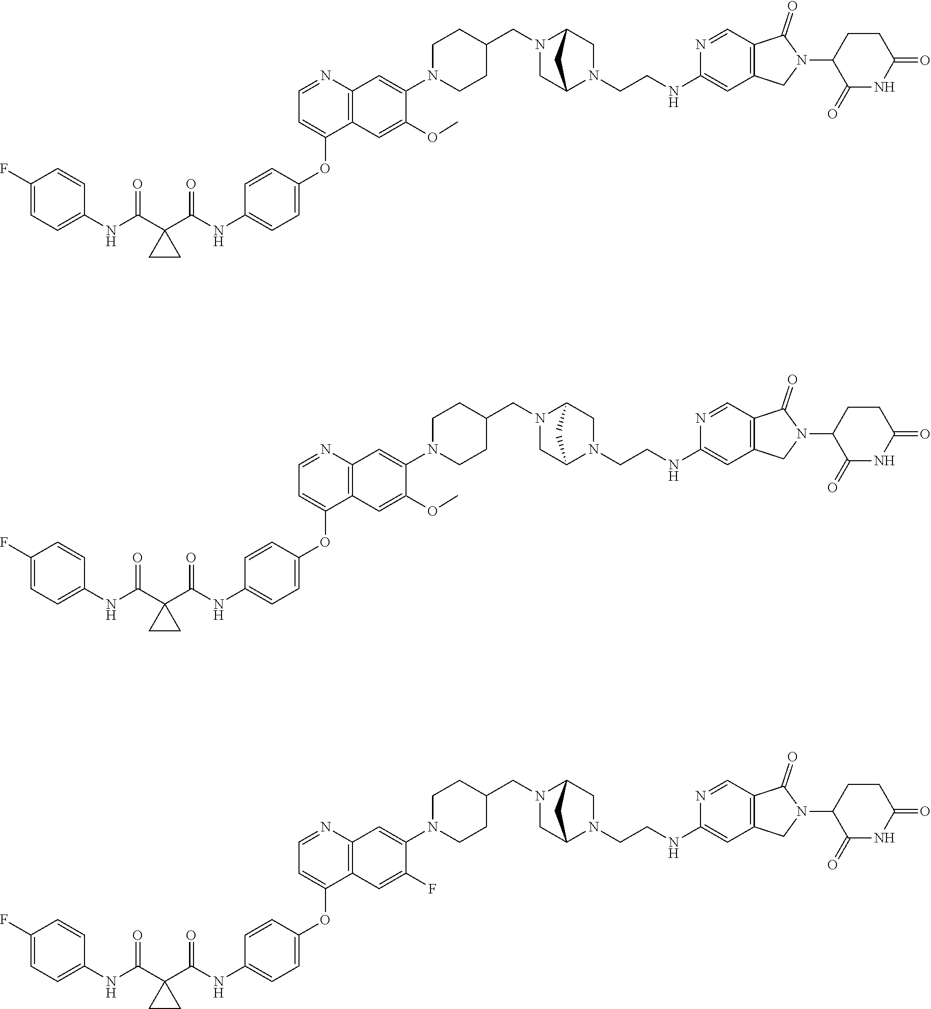 [SMILES: COc1cc2c(Oc3ccc(NC(=O)C4(C(=O)Nc5ccc(F)cc5)CC4)cc3)ccnc2cc1N1CCC(CN2C[C@@H]3C[C@H]2CN3CCNc2cc3c(cn2)C(=O)N(C2CCC(=O)NC2=O)C3)CC1 ; COc1cc2c(Oc3ccc(NC(=O)C4(C(=O)Nc5ccc(F)cc5)CC4)cc3)ccnc2cc1N1CCC(CN2C[C@H]3C[C@@H]2CN3CCNc2cc3c(cn2)C(=O)N(C2CCC(=O)NC2=O)C3)CC1 ; O=C1CCC(N2Cc3cc(NCCN4C[C@@H]5C[C@H]4CN5CC4CCN(c5cc6nccc(Oc7ccc(NC(=O)C8(C(=O)Nc9ccc(F)cc9)CC8)cc7)c6cc5F)CC4)ncc3C2=O)C(=O)N1]

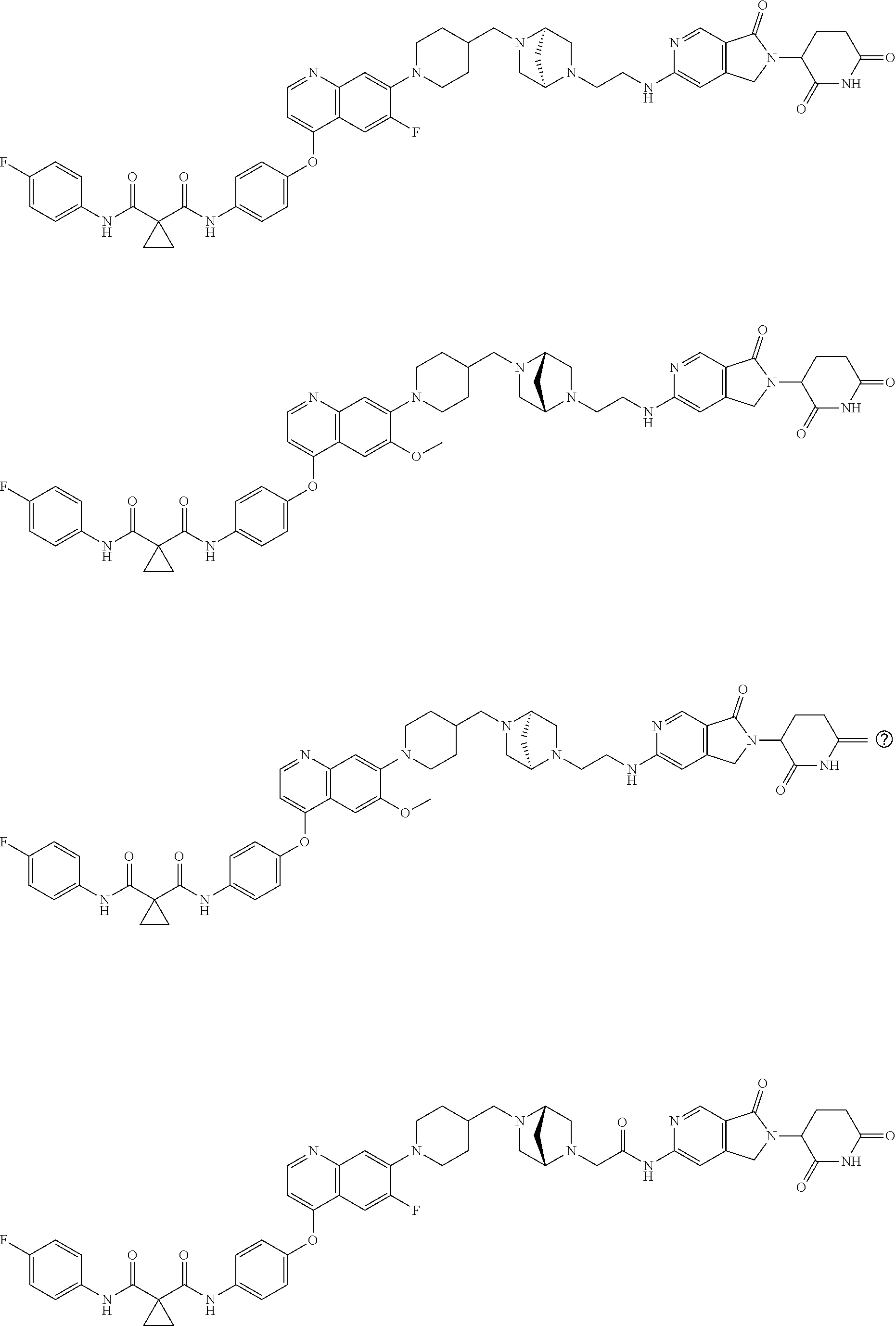

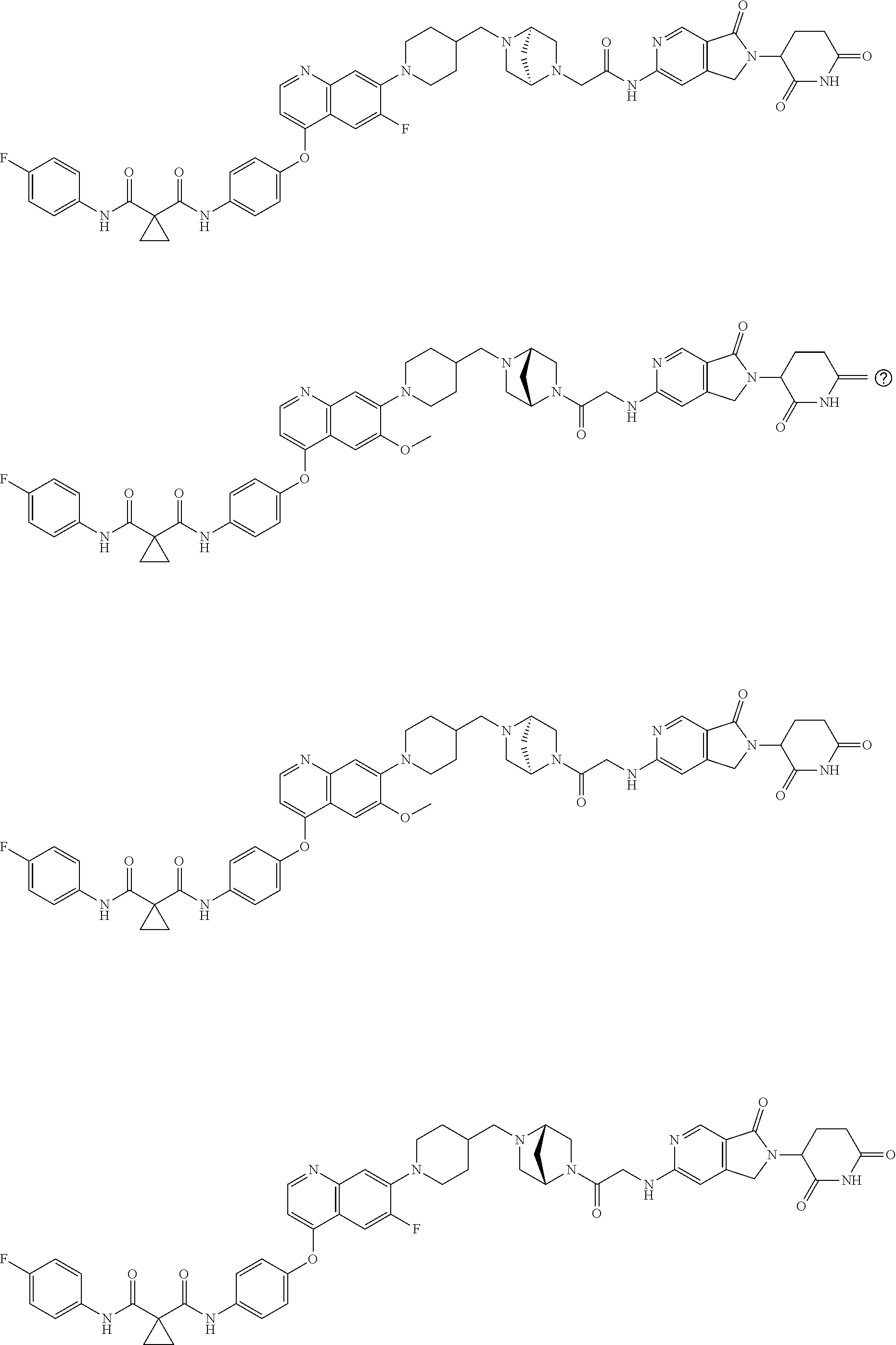

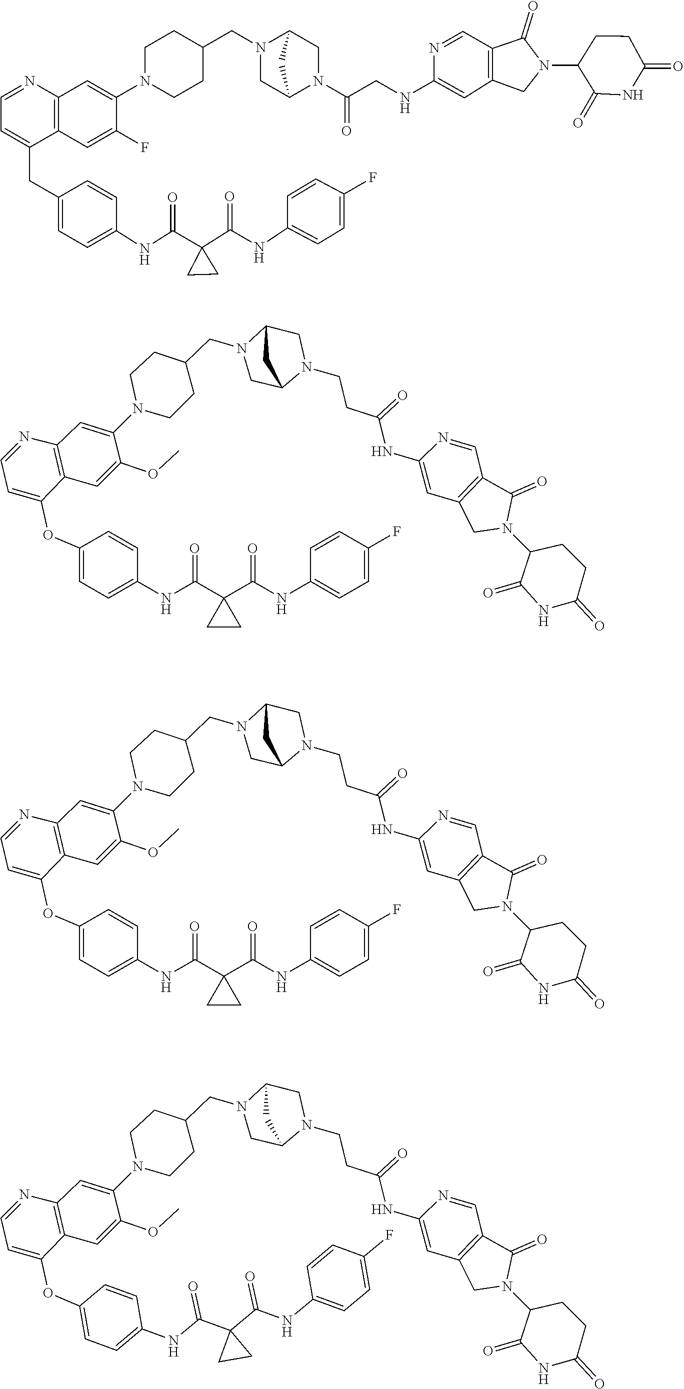

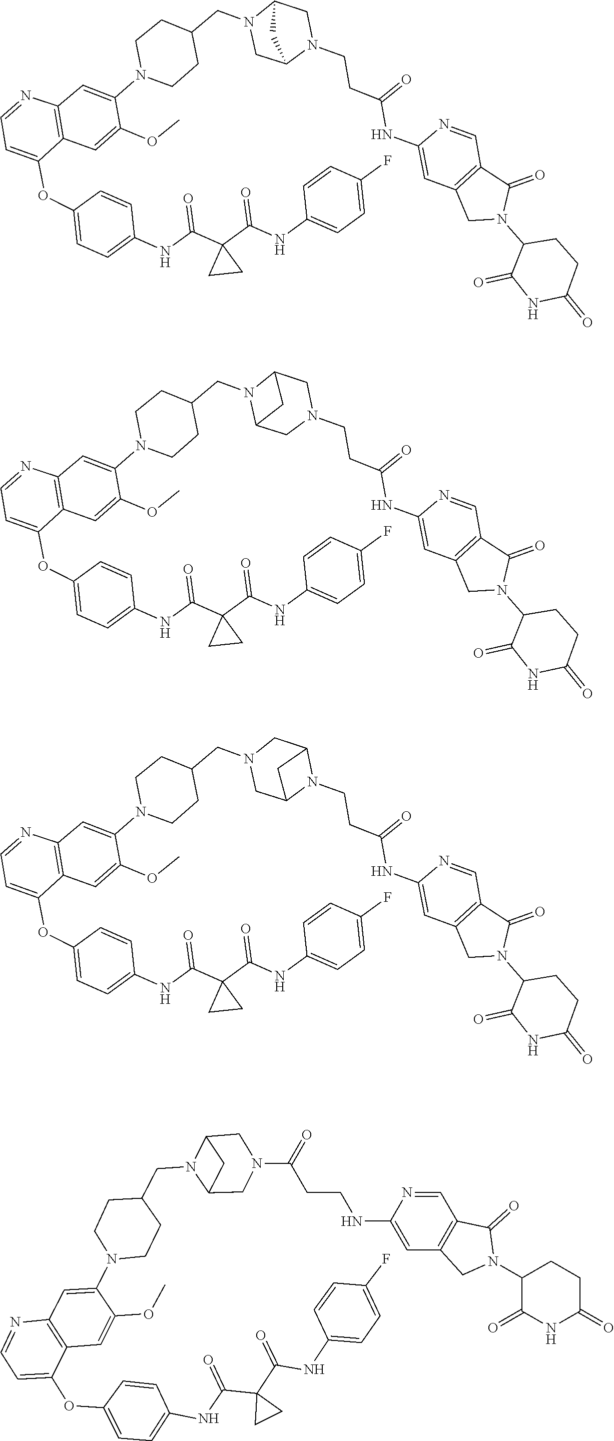

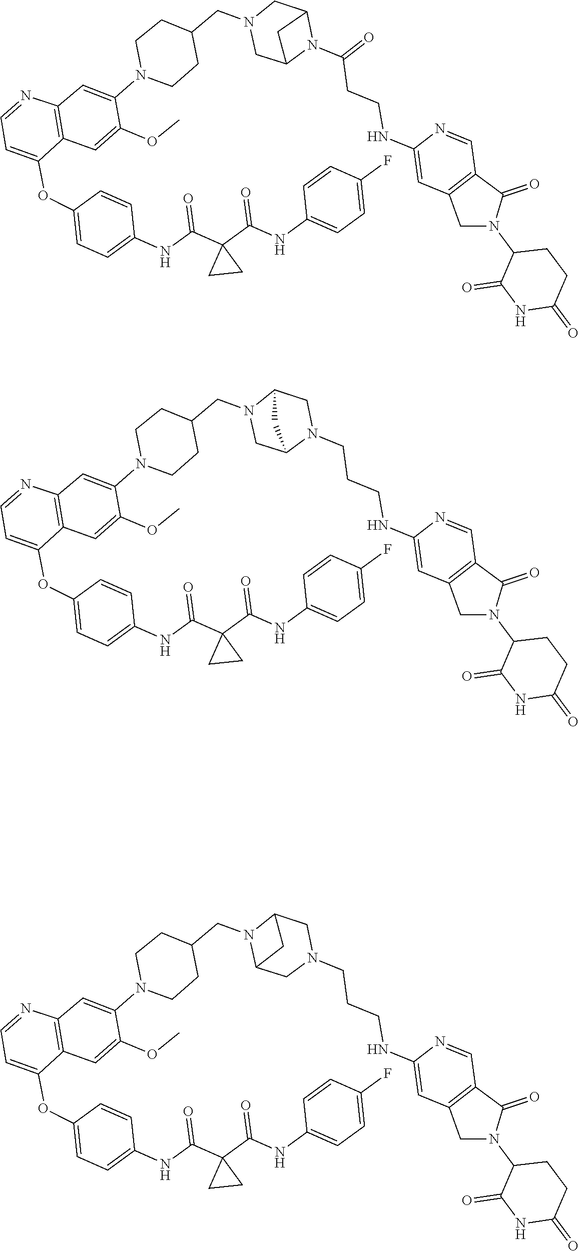 [SMILES: COc1cc2c(Oc3ccc(NC(=O)C4(C(=O)Nc5ccc(F)cc5)CC4)cc3)ccnc2cc1N1CCC(CN2CC3CC(C2)N3C(=O)CCNc2cc3c(cn2)C(=O)N(C2CCC(=O)NC2=O)C3)CC1 ; COc1cc2c(Oc3ccc(NC(=O)C4(C(=O)Nc5ccc(F)cc5)CC4)cc3)ccnc2cc1N1CCC(CN2C[C@H]3C[C@@H]2CN3CCCNc2cc3c(cn2)C(=O)N(C2CCC(=O)NC2=O)C3)CC1 ; COc1cc2c(Oc3ccc(NC(=O)C4(C(=O)Nc5ccc(F)cc5)CC4)cc3)ccnc2cc1N1CCC(CN2C3CC2CN(CCCNc2cc4c(cn2)C(=O)N(C2CCC(=O)NC2=O)C4)C3)CC1]

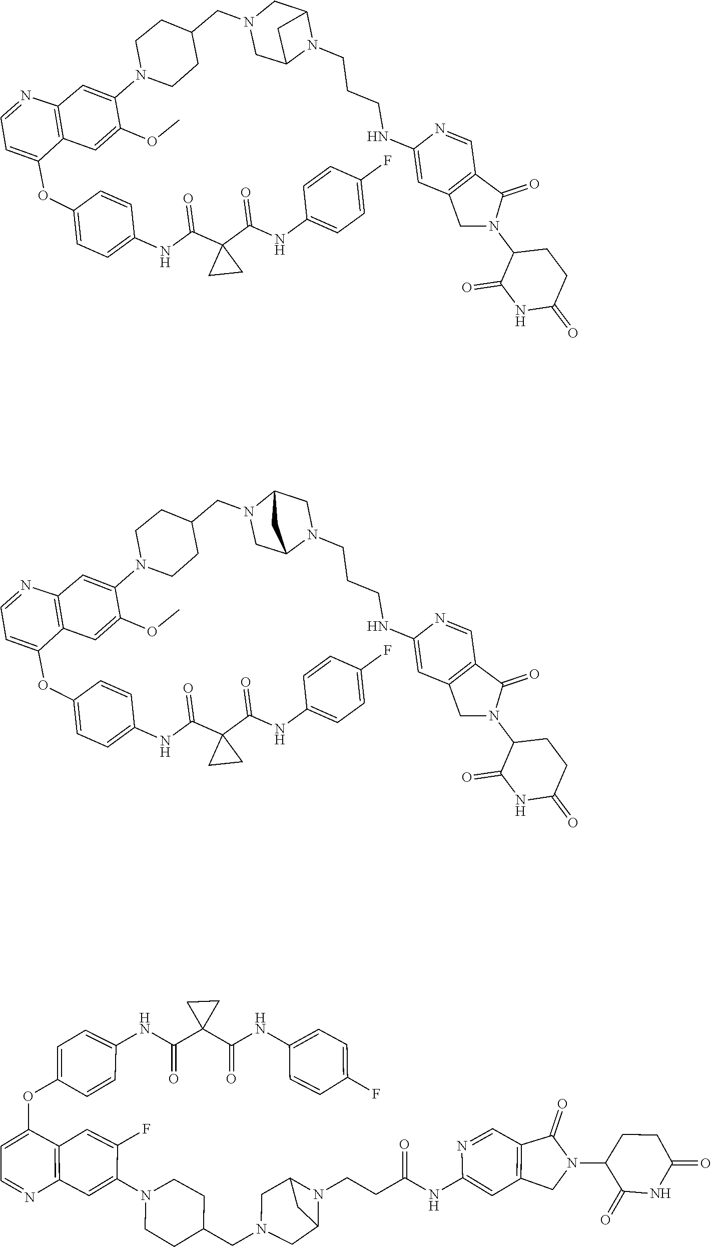 [SMILES: COc1cc2c(Oc3ccc(NC(=O)C4(C(=O)Nc5ccc(F)cc5)CC4)cc3)ccnc2cc1N1CCC(CN2CC3CC(C2)N3CCCNc2cc3c(cn2)C(=O)N(C2CCC(=O)NC2=O)C3)CC1 ; COc1cc2c(Oc3ccc(NC(=O)C4(C(=O)Nc5ccc(F)cc5)CC4)cc3)ccnc2cc1N1CCC(CN2C[C@@H]3C[C@H]2CN3CCCNc2cc3c(cn2)C(=O)N(C2CCC(=O)NC2=O)C3)CC1 ; O=C1CCC(N2Cc3cc(NC(=O)CCN4C5CC4CN(CC4CCN(c6cc7nccc(Oc8ccc(NC(=O)C9(C(=O)Nc%10ccc(F)cc%10)CC9)cc8)c7cc6F)CC4)C5)ncc3C2=O)C(=O)N1]

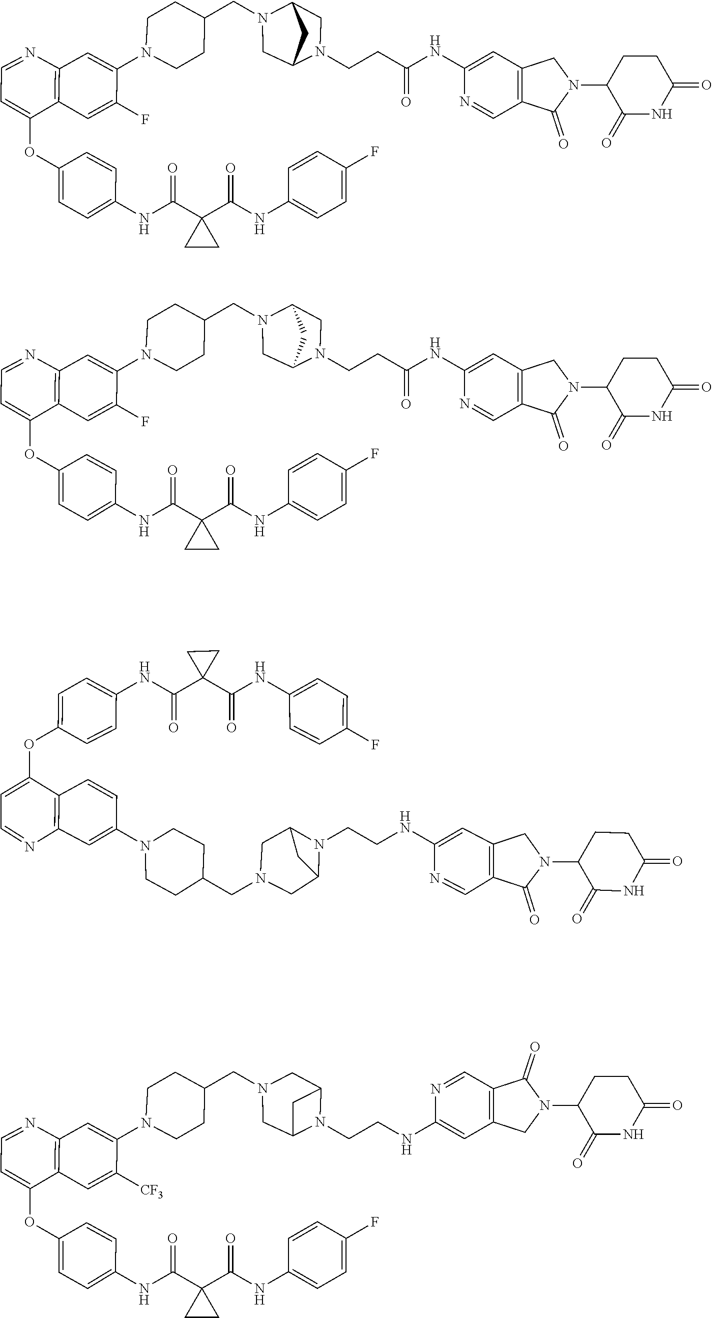

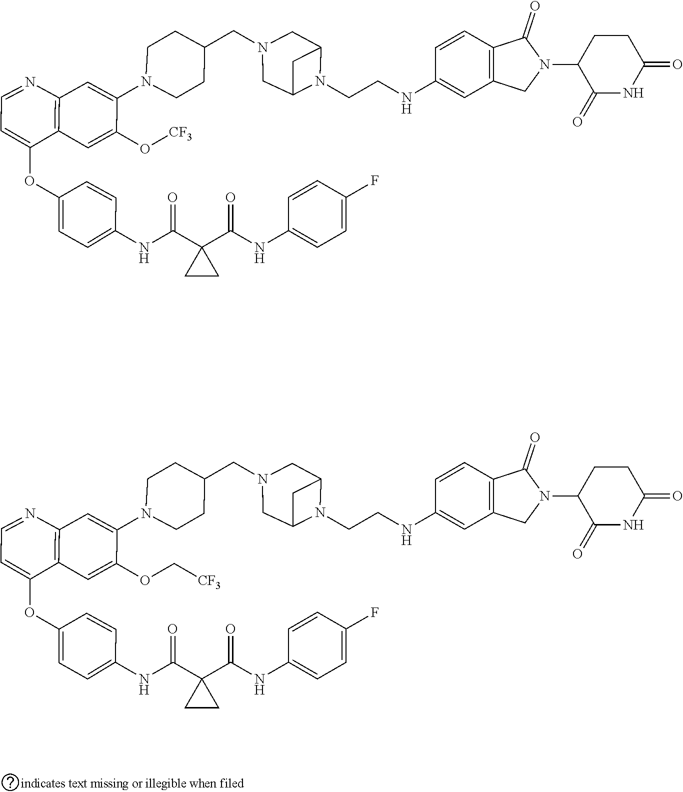 [SMILES: O=C1CCC(N2Cc3cc(NCCN4C5CC4CN(CC4CCN(c6cc7nccc(Oc8ccc(NC(=O)C9(C(=O)Nc%10ccc(F)cc%10)CC9)cc8)c7cc6OC(F)(F)F)CC4)C5)ccc3C2=O)C(=O)N1 ; O=C1CCC(N2Cc3cc(NCCN4C5CC4CN(CC4CCN(c6cc7nccc(Oc8ccc(NC(=O)C9(C(=O)Nc%10ccc(F)cc%10)CC9)cc8)c7cc6OCC(F)(F)F)CC4)C5)ccc3C2=O)C(=O)N1]

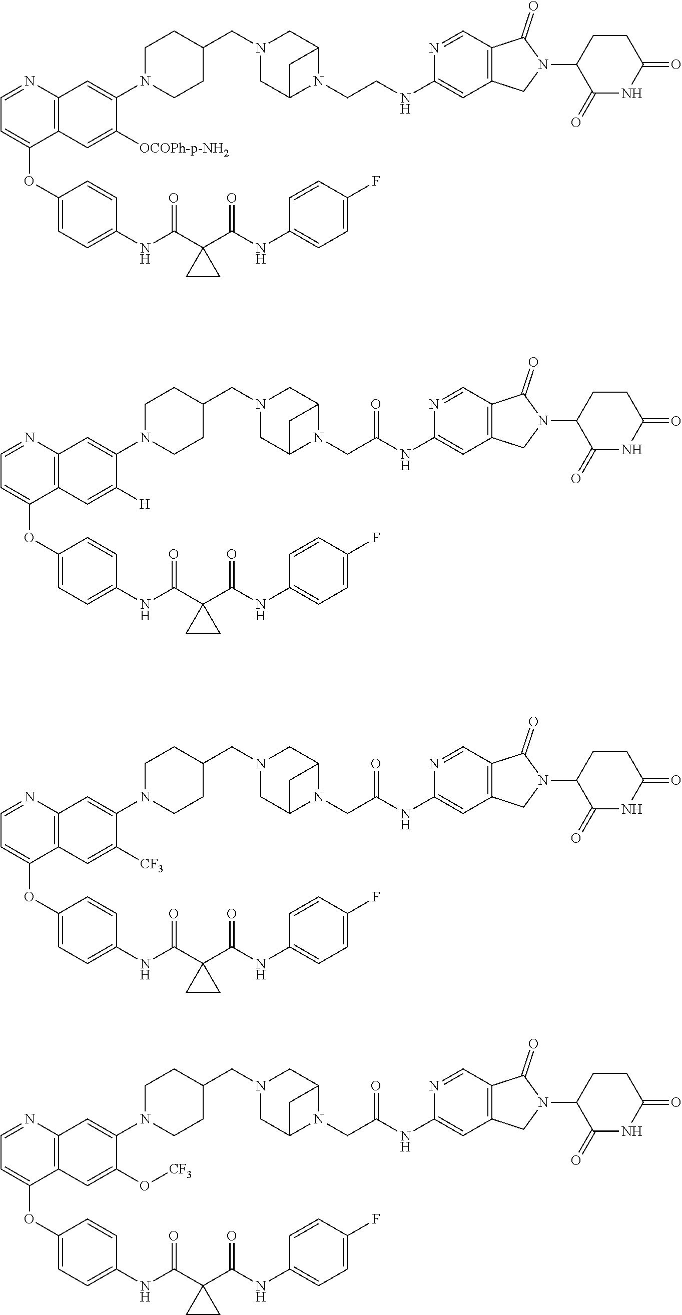

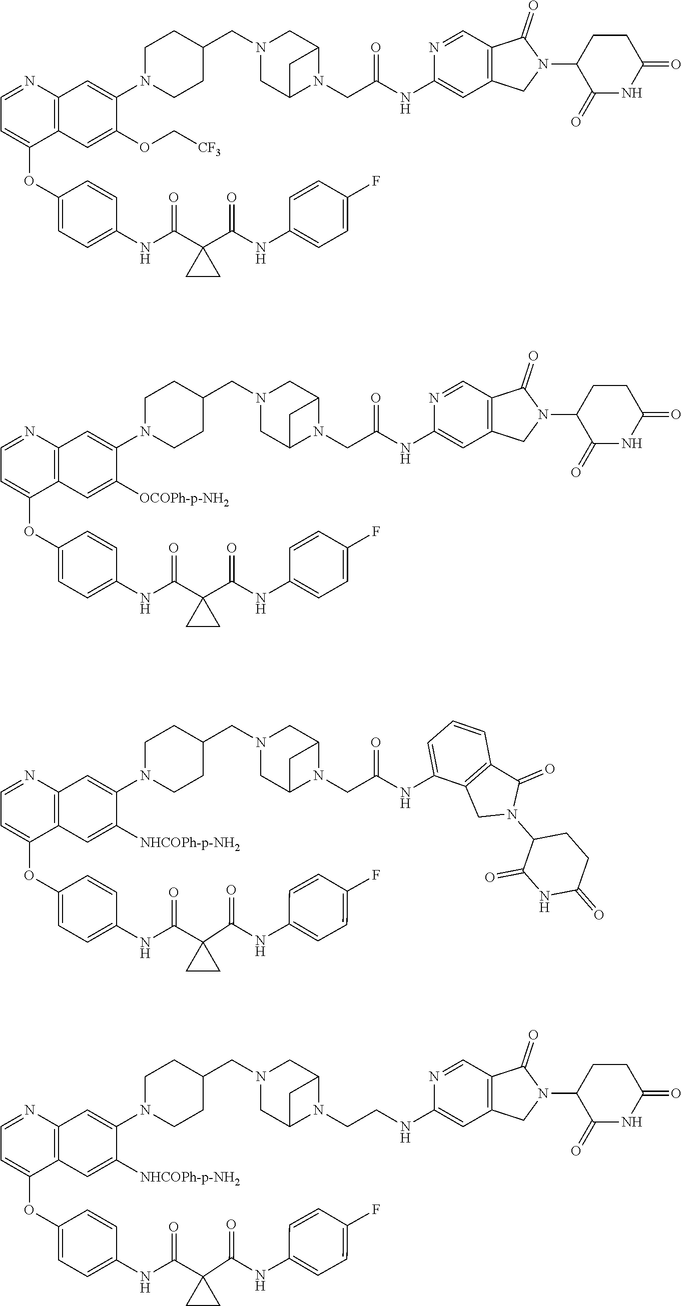

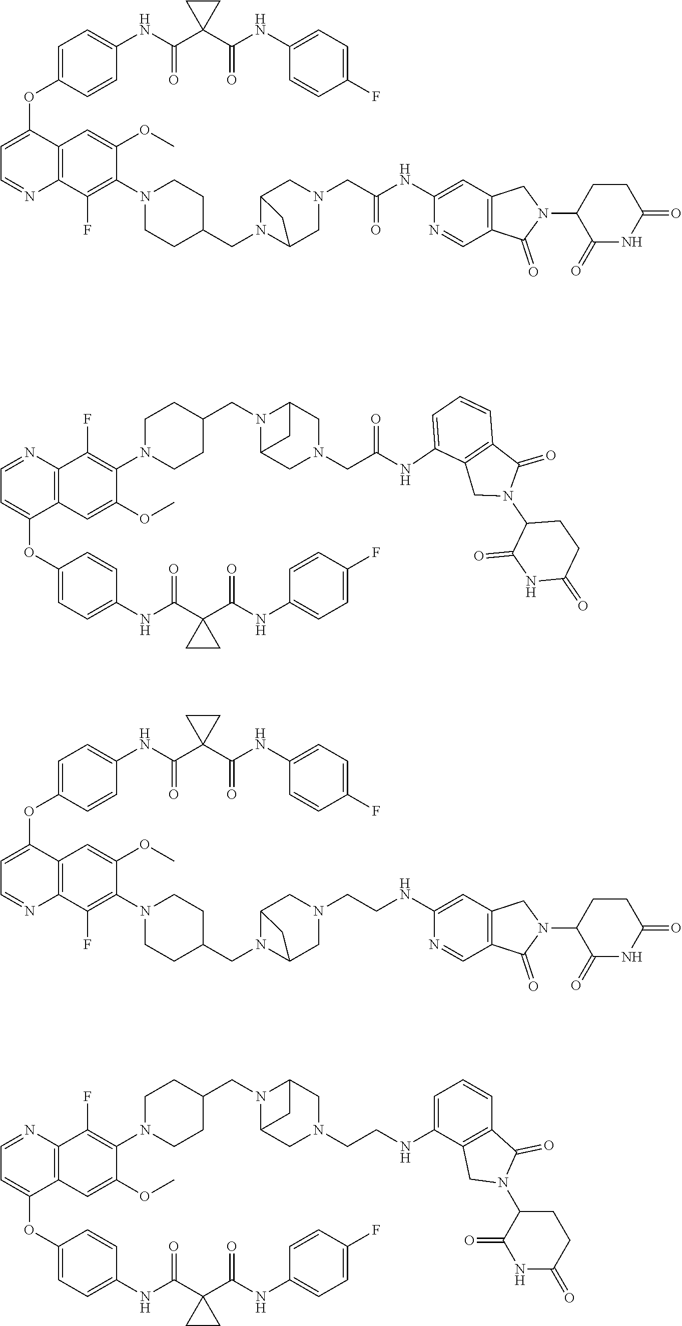

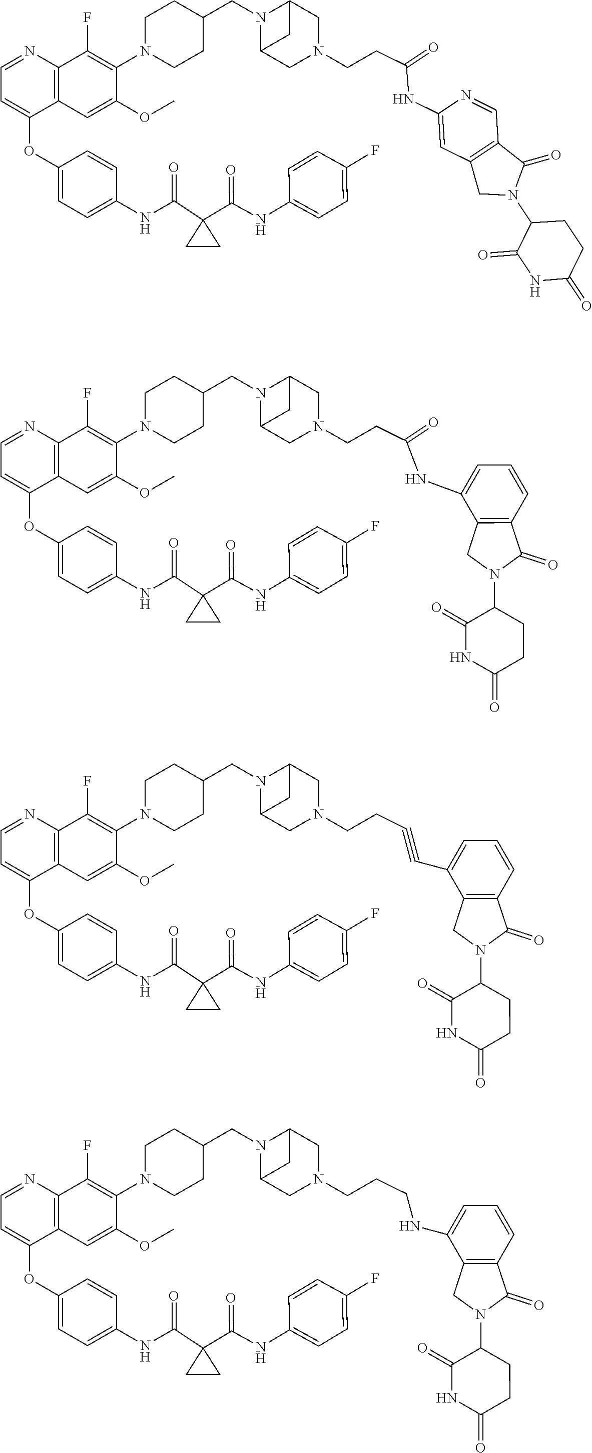

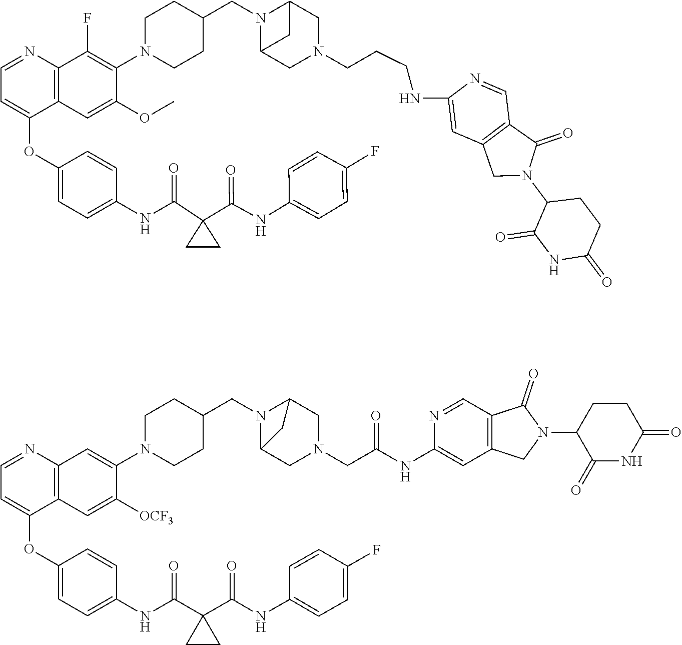 [SMILES: COc1cc2c(Oc3ccc(NC(=O)C4(C(=O)Nc5ccc(F)cc5)CC4)cc3)ccnc2c(F)c1N1CCC(CN2C3CC2CN(CCCNc2cc4c(cn2)C(=O)N(C2CCC(=O)NC2=O)C4)C3)CC1 ; O=C1CCC(N2Cc3cc(NC(=O)CN4CC5CC(C4)N5CC4CCN(c5cc6nccc(Oc7ccc(NC(=O)C8(C(=O)Nc9ccc(F)cc9)CC8)cc7)c6cc5OC(F)(F)F)CC4)ncc3C2=O)C(=O)N1]

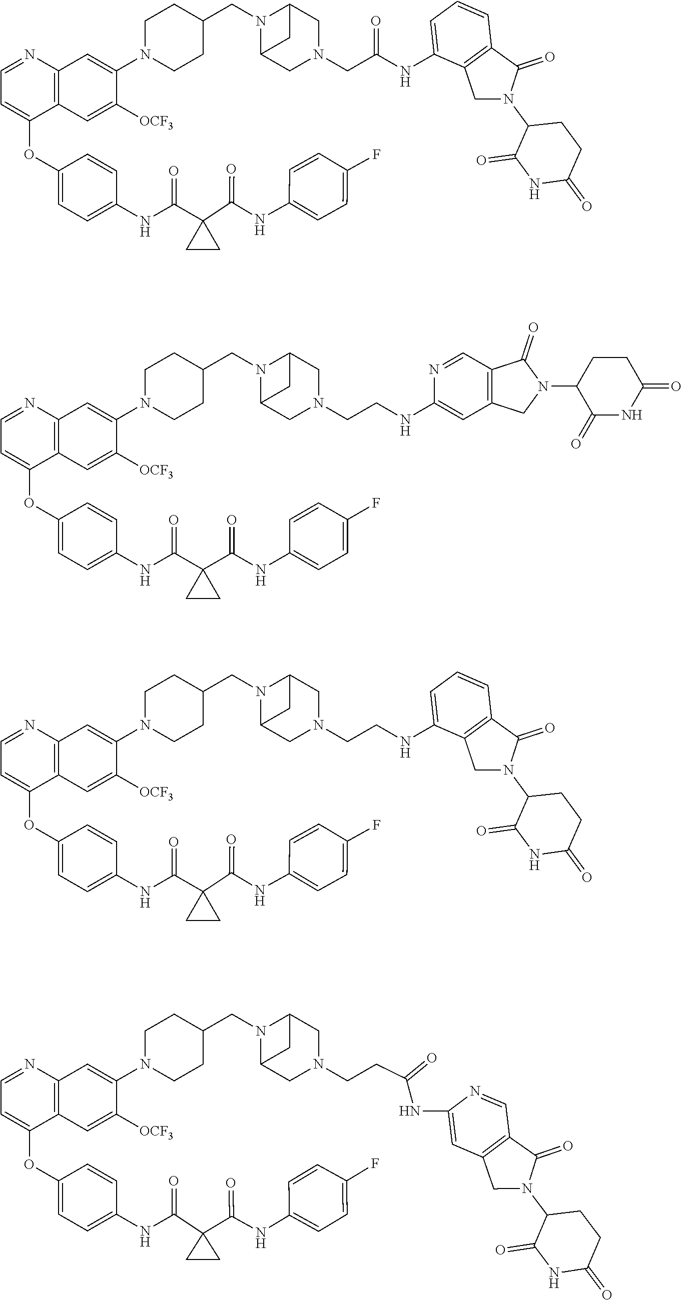

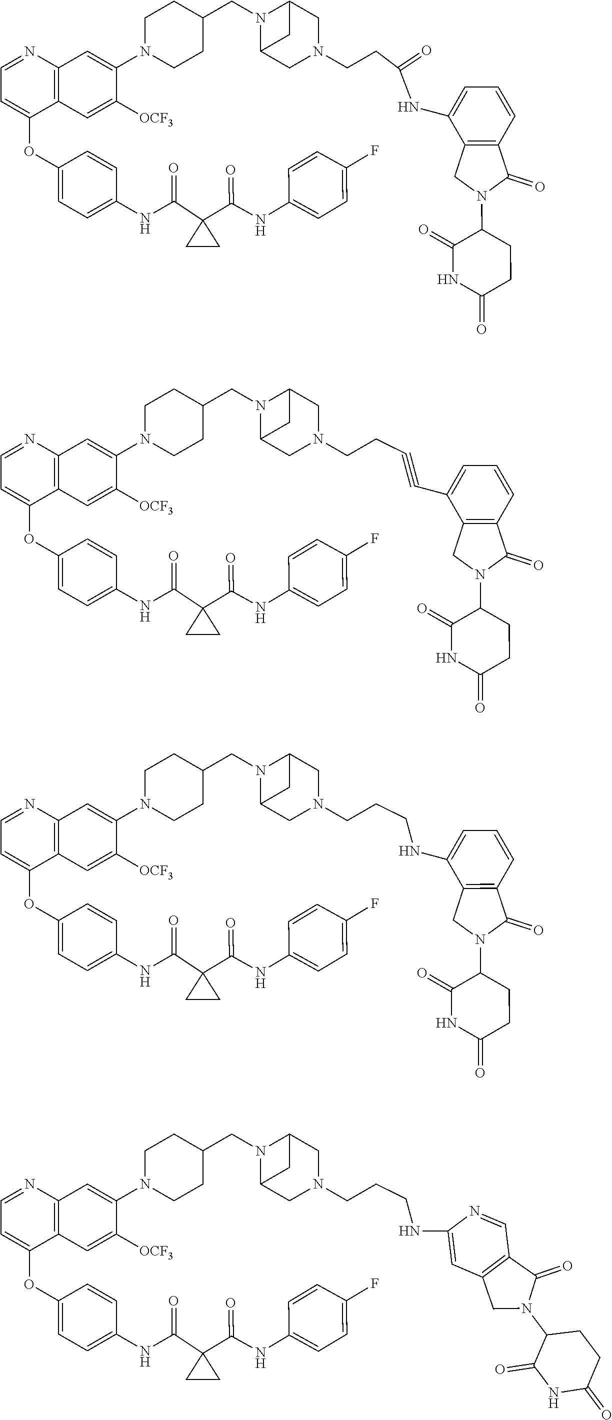

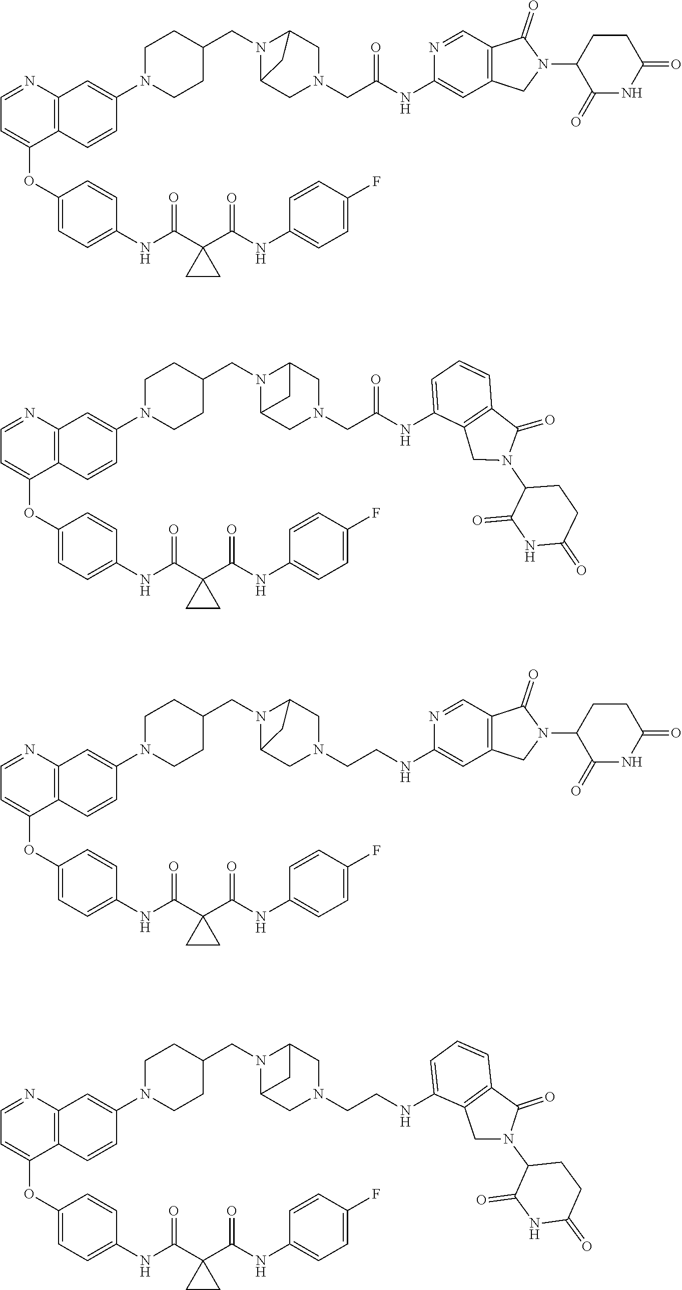 [SMILES: O=C1CCC(N2Cc3cc(NC(=O)CN4CC5CC(C4)N5CC4CCN(c5ccc6c(Oc7ccc(NC(=O)C8(C(=O)Nc9ccc(F)cc9)CC8)cc7)ccnc6c5)CC4)ncc3C2=O)C(=O)N1 ; O=C1CCC(N2Cc3c(NC(=O)CN4CC5CC(C4)N5CC4CCN(c5ccc6c(Oc7ccc(NC(=O)C8(C(=O)Nc9ccc(F)cc9)CC8)cc7)ccnc6c5)CC4)cccc3C2=O)C(=O)N1 ; O=C1CCC(N2Cc3cc(NCCN4CC5CC(C4)N5CC4CCN(c5ccc6c(Oc7ccc(NC(=O)C8(C(=O)Nc9ccc(F)cc9)CC8)cc7)ccnc6c5)CC4)ncc3C2=O)C(=O)N1 ; O=C1CCC(N2Cc3c(NCCN4CC5CC(C4)N5CC4CCN(c5ccc6c(Oc7ccc(NC(=O)C8(C(=O)Nc9ccc(F)cc9)CC8)cc7)ccnc6c5)CC4)cccc3C2=O)C(=O)N1]

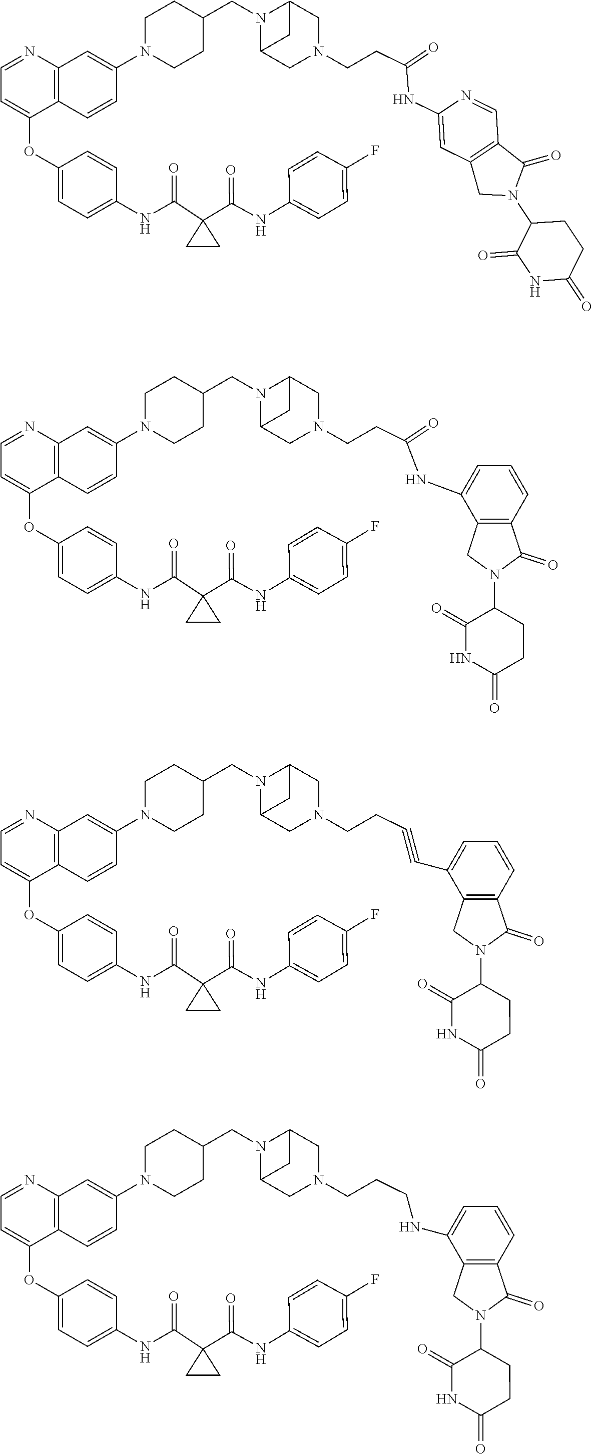 [SMILES: O=C1CCC(N2Cc3cc(NC(=O)CCN4CC5CC(C4)N5CC4CCN(c5ccc6c(Oc7ccc(NC(=O)C8(C(=O)Nc9ccc(F)cc9)CC8)cc7)ccnc6c5)CC4)ncc3C2=O)C(=O)N1 ; O=C1CCC(N2Cc3c(NC(=O)CCN4CC5CC(C4)N5CC4CCN(c5ccc6c(Oc7ccc(NC(=O)C8(C(=O)Nc9ccc(F)cc9)CC8)cc7)ccnc6c5)CC4)cccc3C2=O)C(=O)N1 ; O=C1CCC(N2Cc3c(C#CCCN4CC5CC(C4)N5CC4CCN(c5ccc6c(Oc7ccc(NC(=O)C8(C(=O)Nc9ccc(F)cc9)CC8)cc7)ccnc6c5)CC4)cccc3C2=O)C(=O)N1 ; O=C1CCC(N2Cc3c(NCCCN4CC5CC(C4)N5CC4CCN(c5ccc6c(Oc7ccc(NC(=O)C8(C(=O)Nc9ccc(F)cc9)CC8)cc7)ccnc6c5)CC4)cccc3C2=O)C(=O)N1]

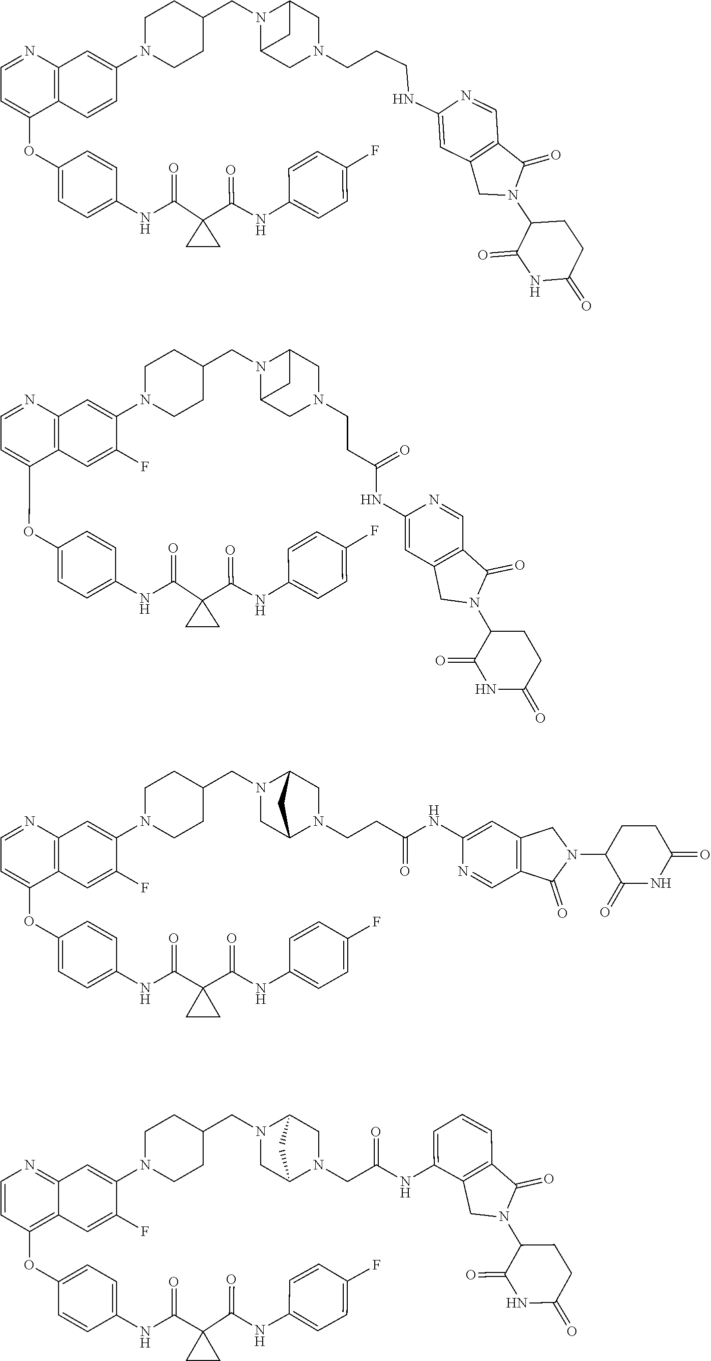

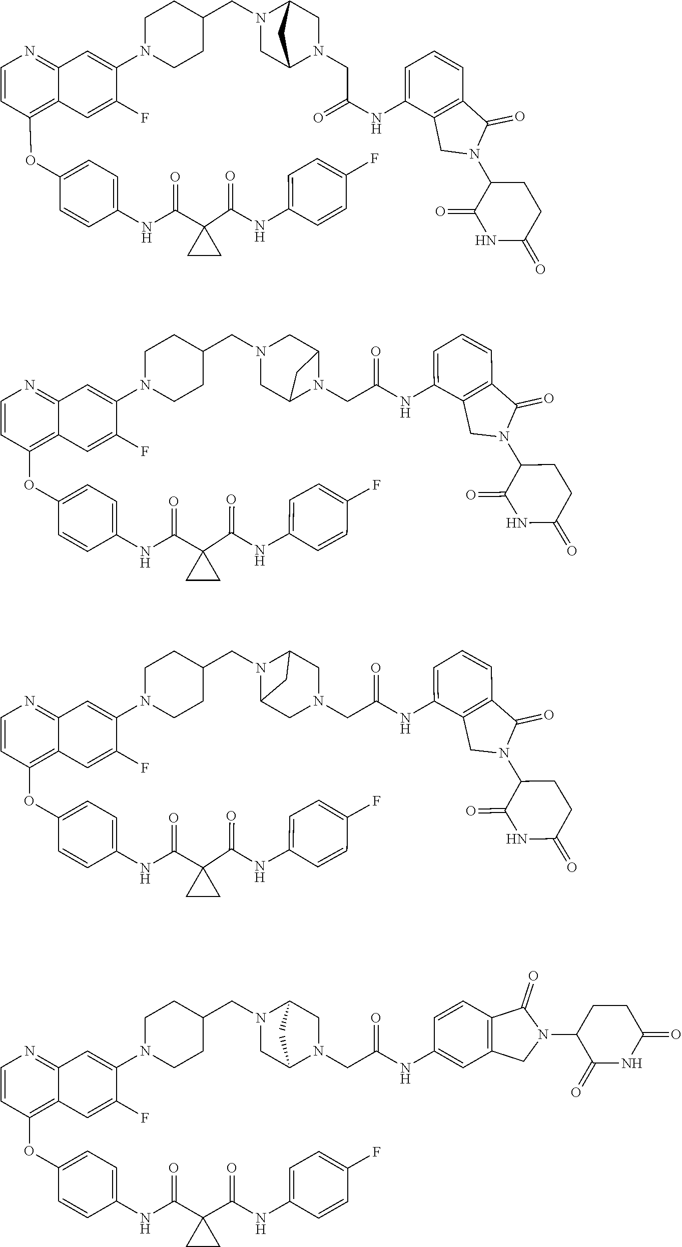

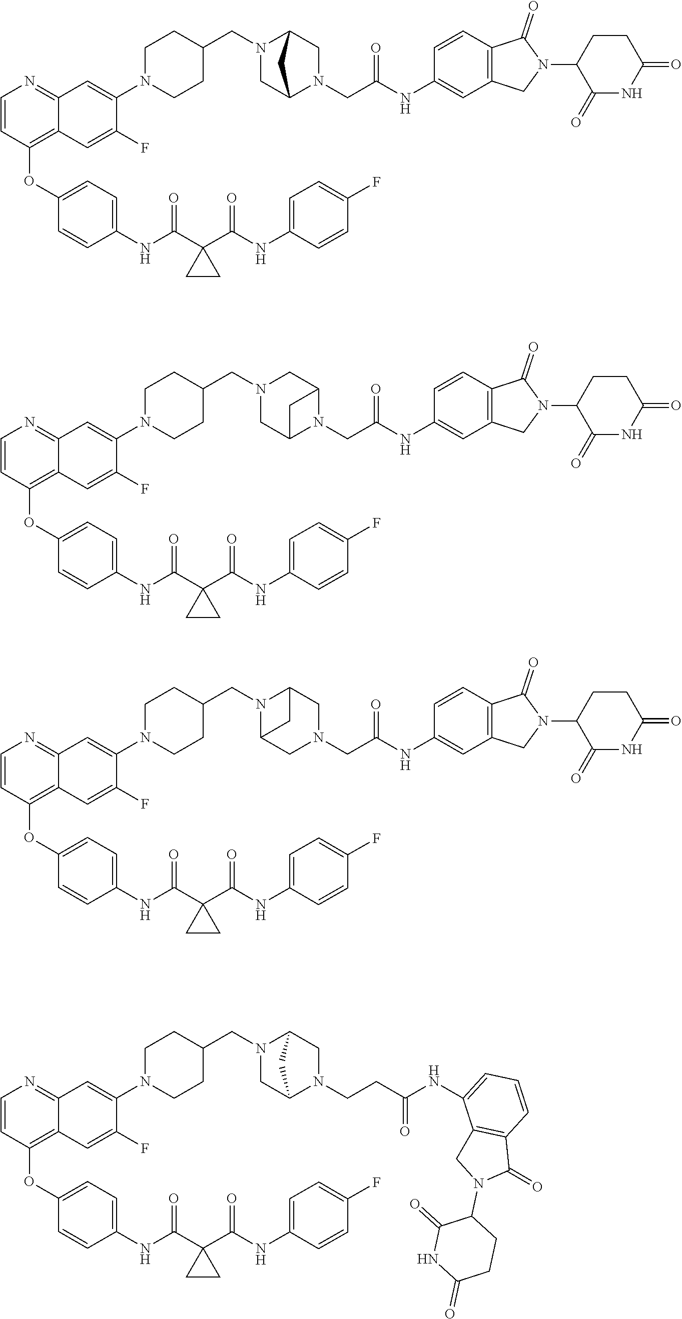

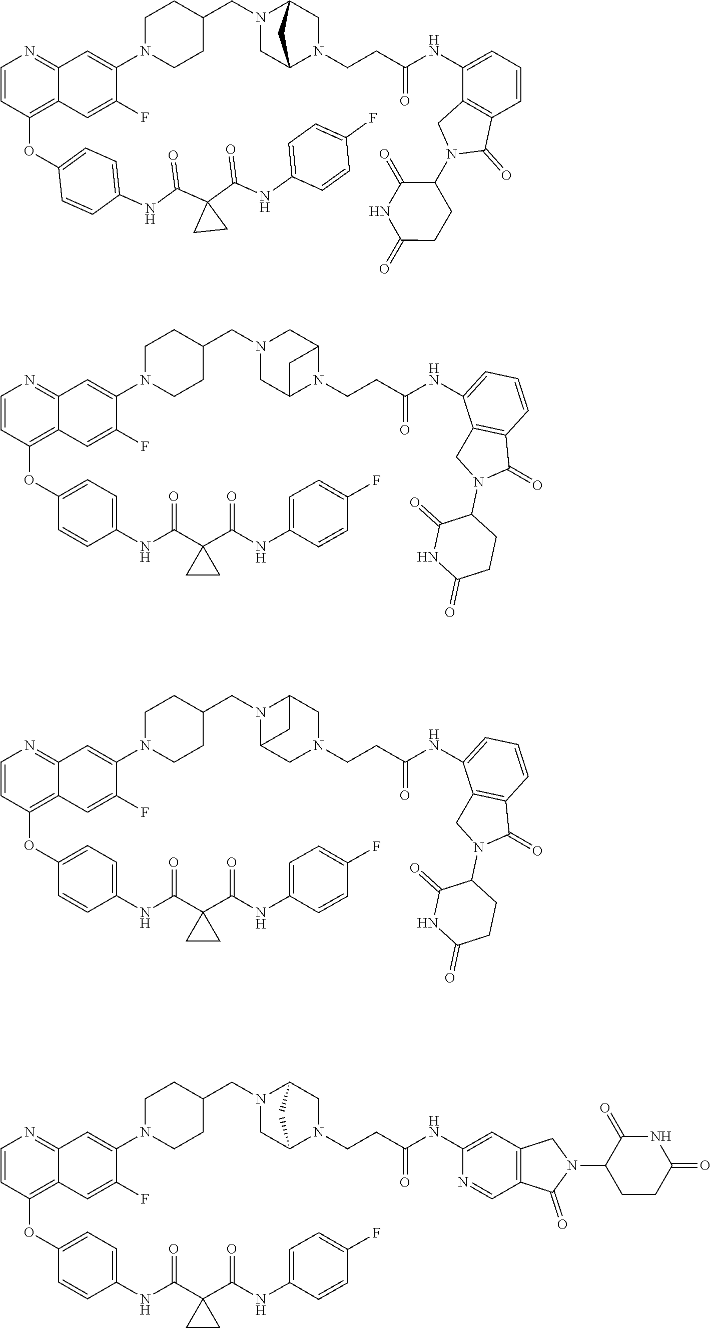

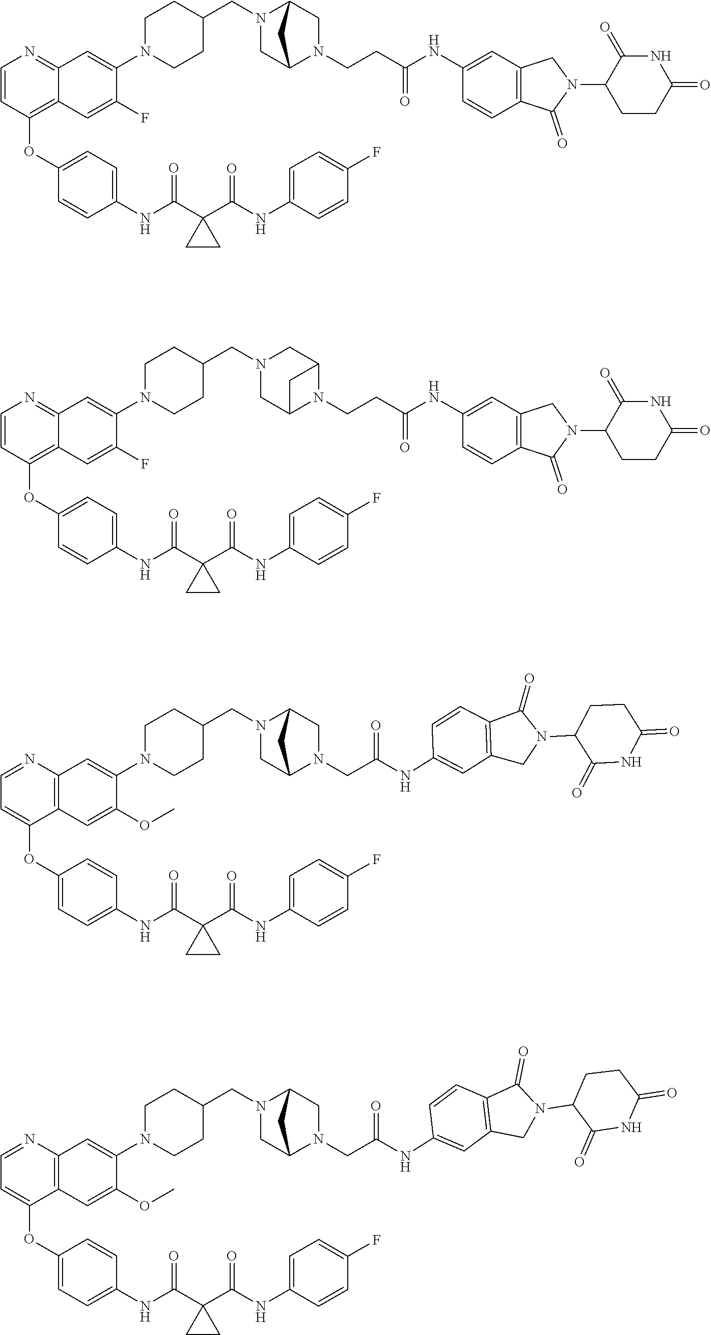

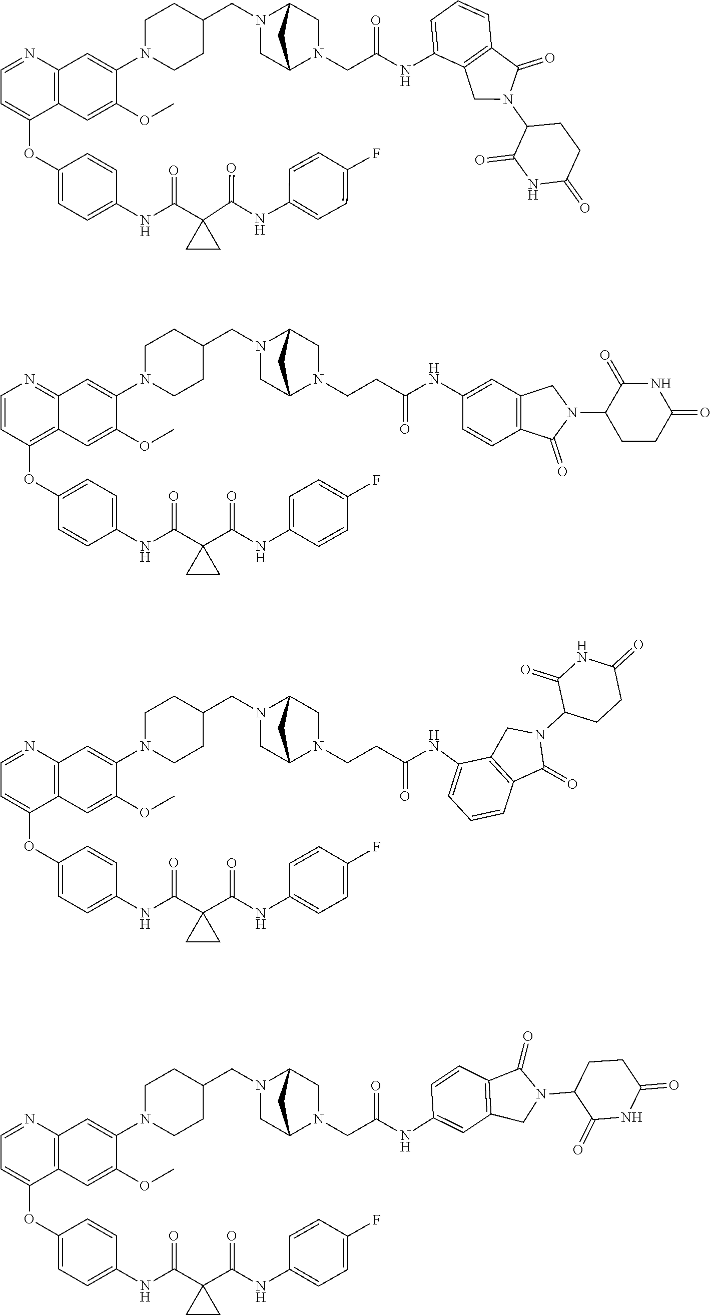

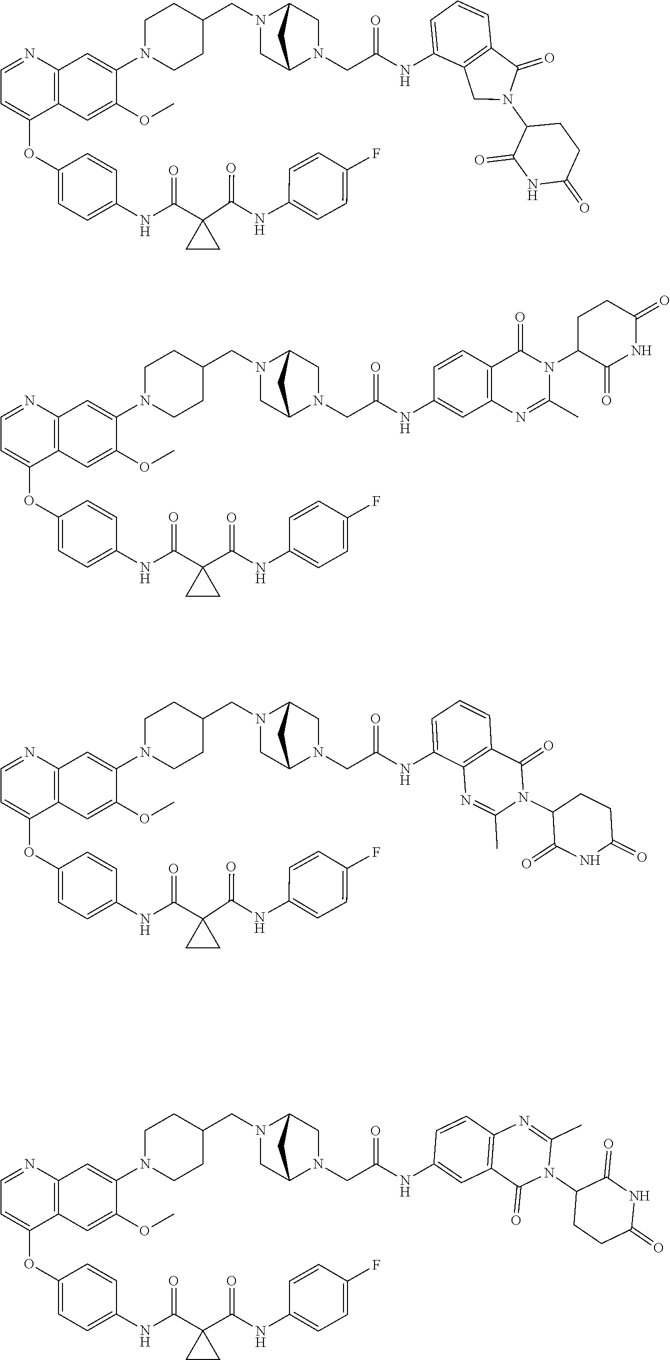

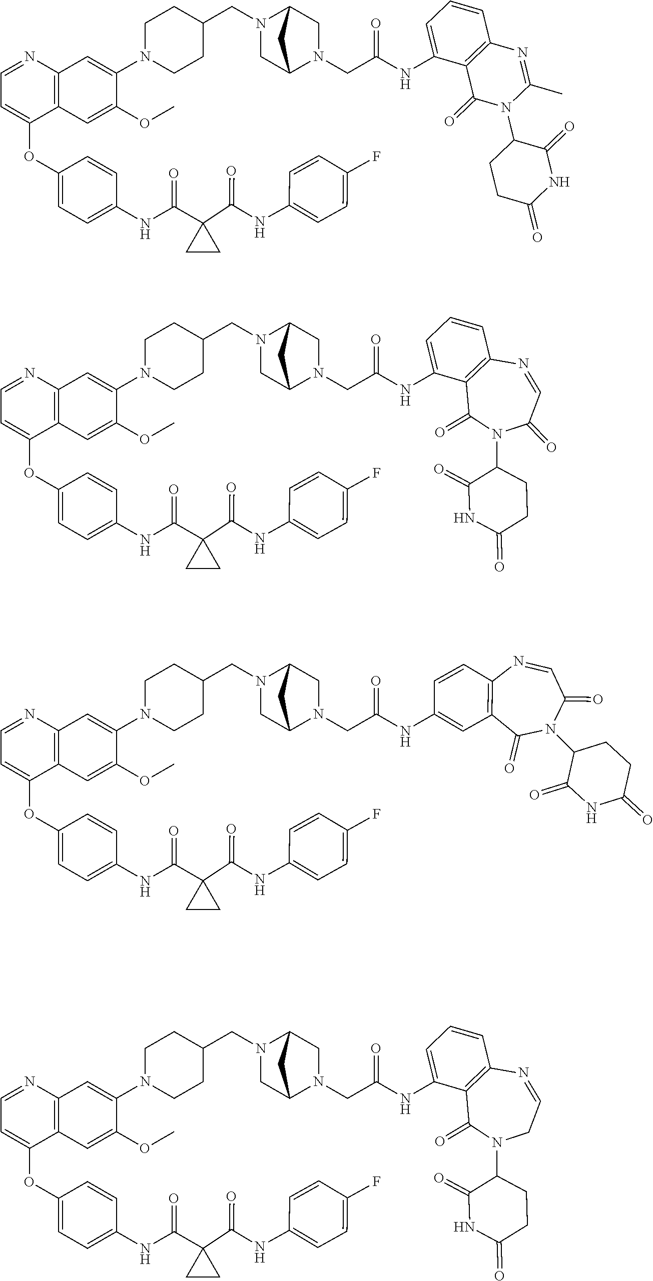

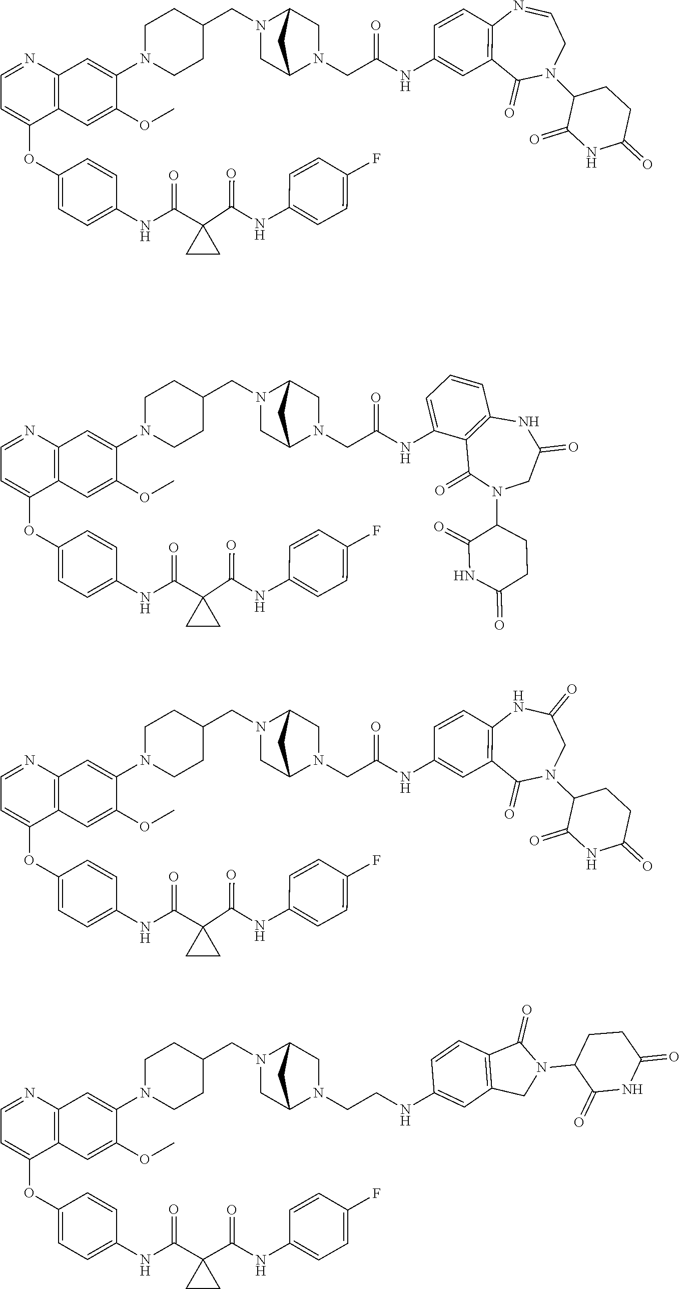

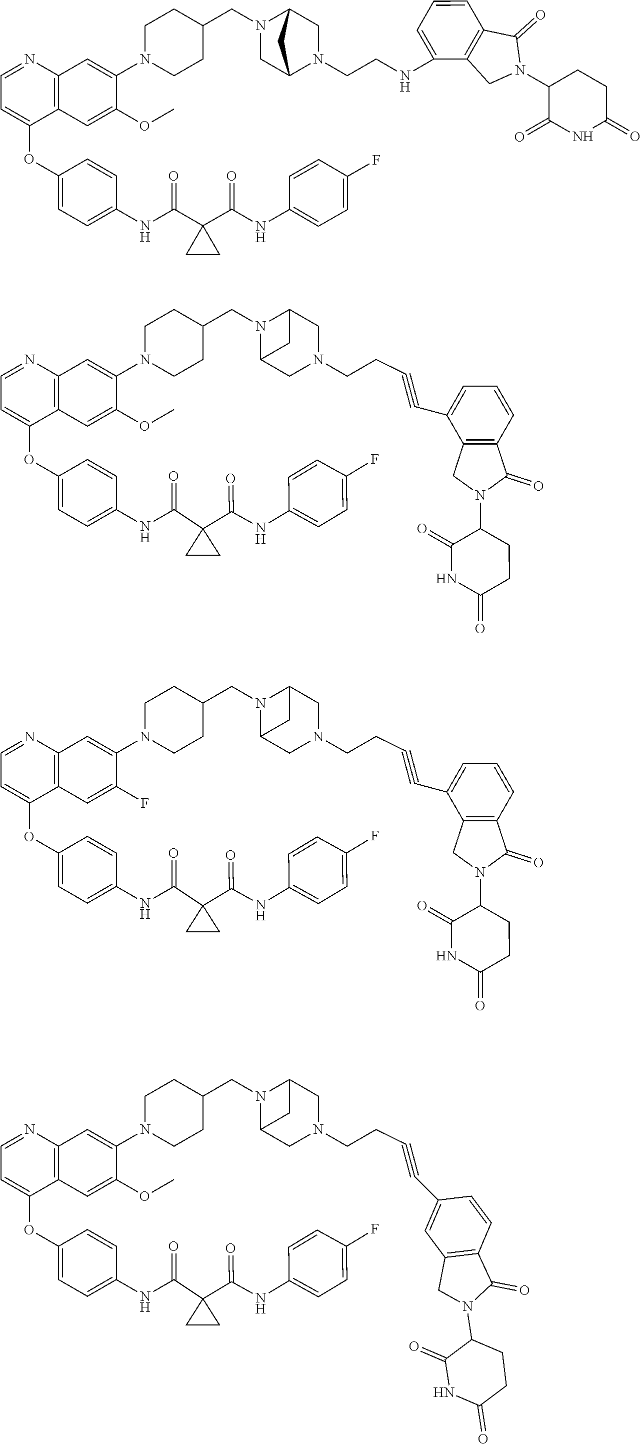 [SMILES: COc1cc2c(Oc3ccc(NC(=O)C4(C(=O)Nc5ccc(F)cc5)CC4)cc3)ccnc2cc1N1CCC(CN2C[C@@H]3C[C@H]2CN3CCNc2cccc3c2CN(C2CCC(=O)NC2=O)C3=O)CC1 ; COc1cc2c(Oc3ccc(NC(=O)C4(C(=O)Nc5ccc(F)cc5)CC4)cc3)ccnc2cc1N1CCC(CN2C3CC2CN(CCC#Cc2cccc4c2CN(C2CCC(=O)NC2=O)C4=O)C3)CC1 ; O=C1CCC(N2Cc3c(C#CCCN4CC5CC(C4)N5CC4CCN(c5cc6nccc(Oc7ccc(NC(=O)C8(C(=O)Nc9ccc(F)cc9)CC8)cc7)c6cc5F)CC4)cccc3C2=O)C(=O)N1 ; COc1cc2c(Oc3ccc(NC(=O)C4(C(=O)Nc5ccc(F)cc5)CC4)cc3)ccnc2cc1N1CCC(CN2C3CC2CN(CCC#Cc2ccc4c(c2)CN(C2CCC(=O)NC2=O)C4=O)C3)CC1]

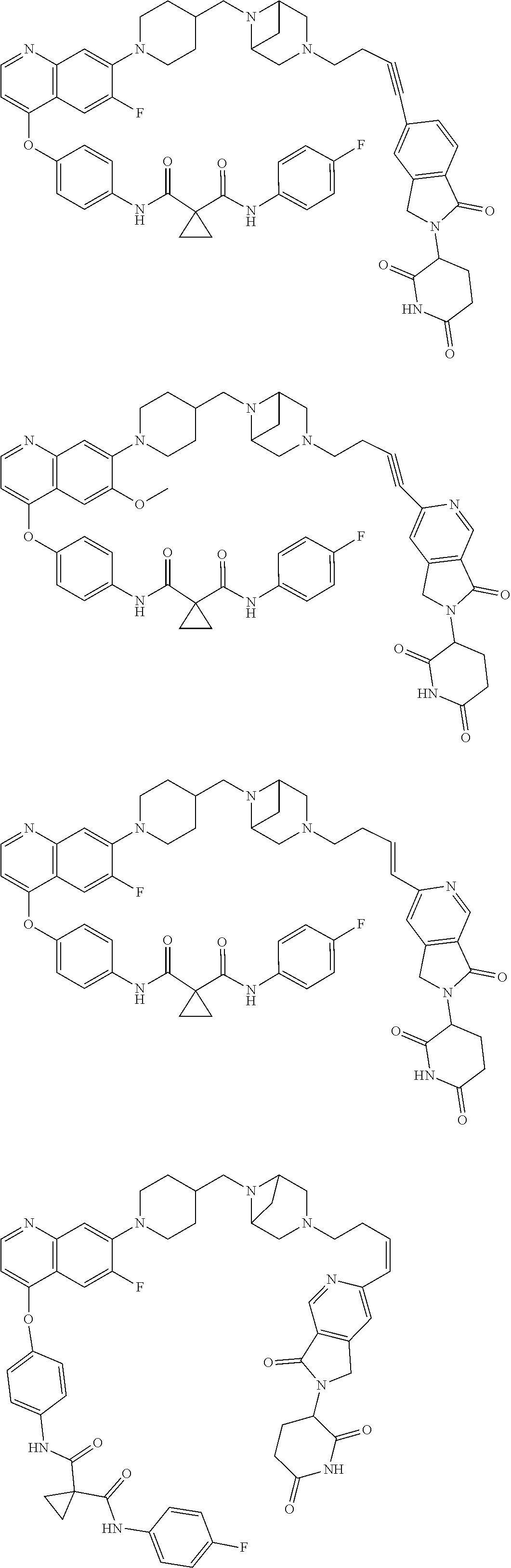 [SMILES: O=C1CCC(N2Cc3cc(C#CCCN4CC5CC(C4)N5CC4CCN(c5cc6nccc(Oc7ccc(NC(=O)C8(C(=O)Nc9ccc(F)cc9)CC8)cc7)c6cc5F)CC4)ccc3C2=O)C(=O)N1 ; COc1cc2c(Oc3ccc(NC(=O)C4(C(=O)Nc5ccc(F)cc5)CC4)cc3)ccnc2cc1N1CCC(CN2C3CC2CN(CCC#Cc2cc4c(cn2)C(=O)N(C2CCC(=O)NC2=O)C4)C3)CC1 ; O=C1CCC(N2Cc3cc(/C=C/CCN4CC5CC(C4)N5CC4CCN(c5cc6nccc(Oc7ccc(NC(=O)C8(C(=O)Nc9ccc(F)cc9)CC8)cc7)c6cc5F)CC4)ncc3C2=O)C(=O)N1 ; O=C1CCC(N2Cc3cc(/C=C\CCN4CC5CC(C4)N5CC4CCN(c5cc6nccc(Oc7ccc(NC(=O)C8(C(=O)Nc9ccc(F)cc9)CC8)cc7)c6cc5F)CC4)ncc3C2=O)C(=O)N1]

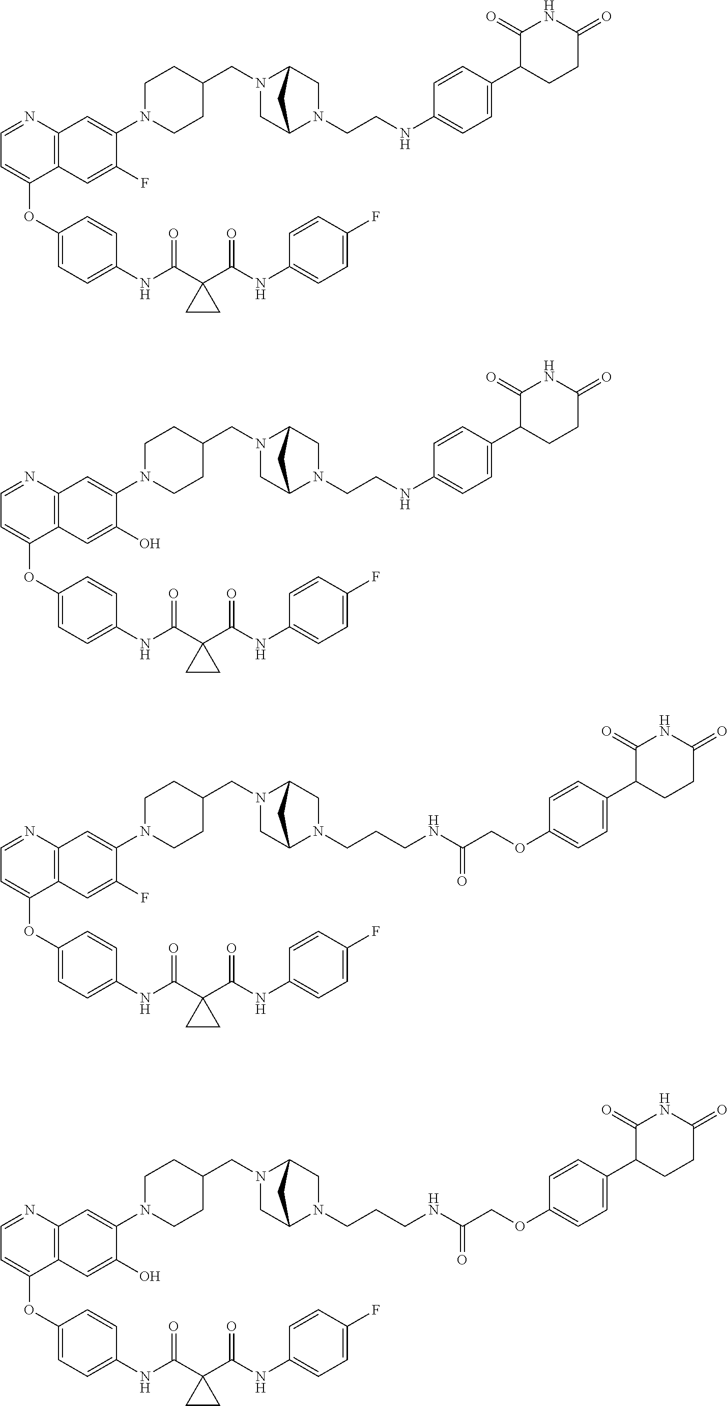 [SMILES: O=C1CCC(c2ccc(NCCN3C[C@@H]4C[C@H]3CN4CC3CCN(c4cc5nccc(Oc6ccc(NC(=O)C7(C(=O)Nc8ccc(F)cc8)CC7)cc6)c5cc4F)CC3)cc2)C(=O)N1 ; O=C1CCC(c2ccc(NCCN3C[C@@H]4C[C@H]3CN4CC3CCN(c4cc5nccc(Oc6ccc(NC(=O)C7(C(=O)Nc8ccc(F)cc8)CC7)cc6)c5cc4O)CC3)cc2)C(=O)N1 ; O=C(COc1ccc(C2CCC(=O)NC2=O)cc1)NCCCN1C[C@@H]2C[C@H]1CN2CC1CCN(c2cc3nccc(Oc4ccc(NC(=O)C5(C(=O)Nc6ccc(F)cc6)CC5)cc4)c3cc2F)CC1 ; O=C(COc1ccc(C2CCC(=O)NC2=O)cc1)NCCCN1C[C@@H]2C[C@H]1CN2CC1CCN(c2cc3nccc(Oc4ccc(NC(=O)C5(C(=O)Nc6ccc(F)cc6)CC5)cc4)c3cc2O)CC1]

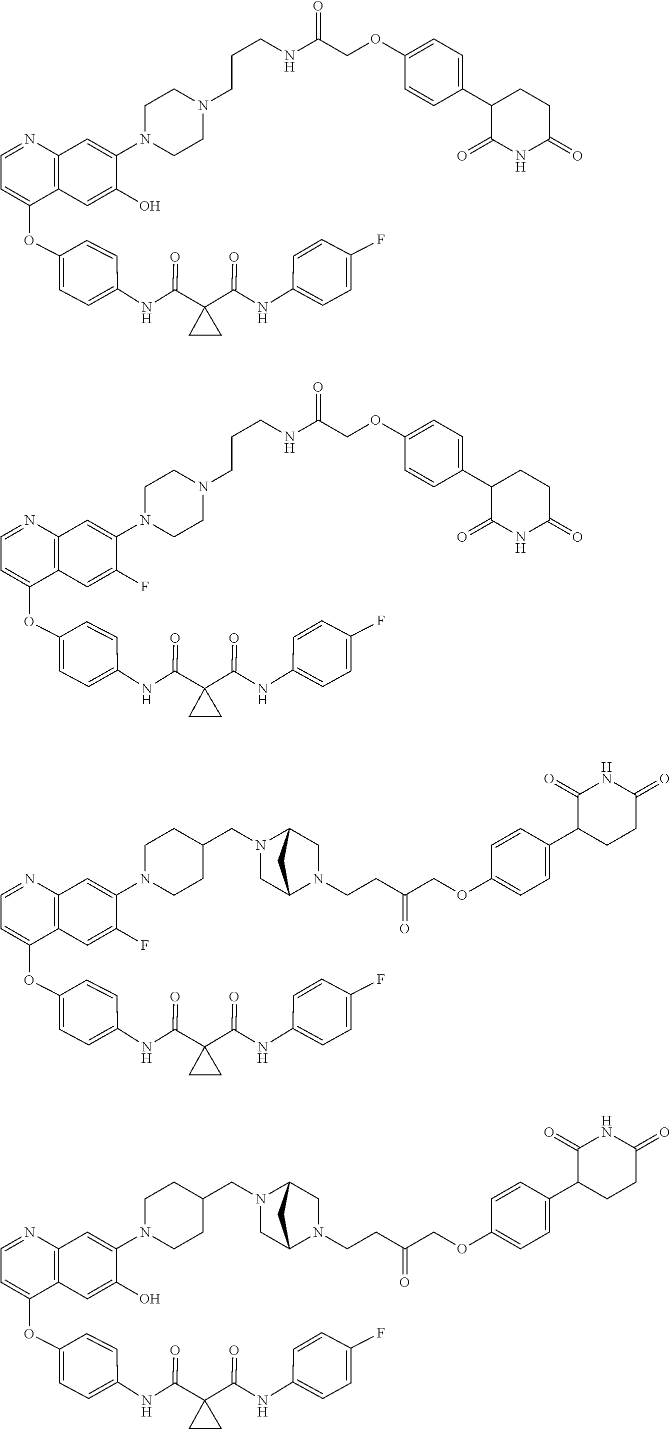 [SMILES: O=C(COc1ccc(C2CCC(=O)NC2=O)cc1)NCCCN1CCN(c2cc3nccc(Oc4ccc(NC(=O)C5(C(=O)Nc6ccc(F)cc6)CC5)cc4)c3cc2O)CC1 ; O=C(COc1ccc(C2CCC(=O)NC2=O)cc1)NCCCN1CCN(c2cc3nccc(Oc4ccc(NC(=O)C5(C(=O)Nc6ccc(F)cc6)CC5)cc4)c3cc2F)CC1 ; O=C(CCN1C[C@@H]2C[C@H]1CN2CC1CCN(c2cc3nccc(Oc4ccc(NC(=O)C5(C(=O)Nc6ccc(F)cc6)CC5)cc4)c3cc2F)CC1)COc1ccc(C2CCC(=O)NC2=O)cc1 ; O=C(CCN1C[C@@H]2C[C@H]1CN2CC1CCN(c2cc3nccc(Oc4ccc(NC(=O)C5(C(=O)Nc6ccc(F)cc6)CC5)cc4)c3cc2O)CC1)COc1ccc(C2CCC(=O)NC2=O)cc1]

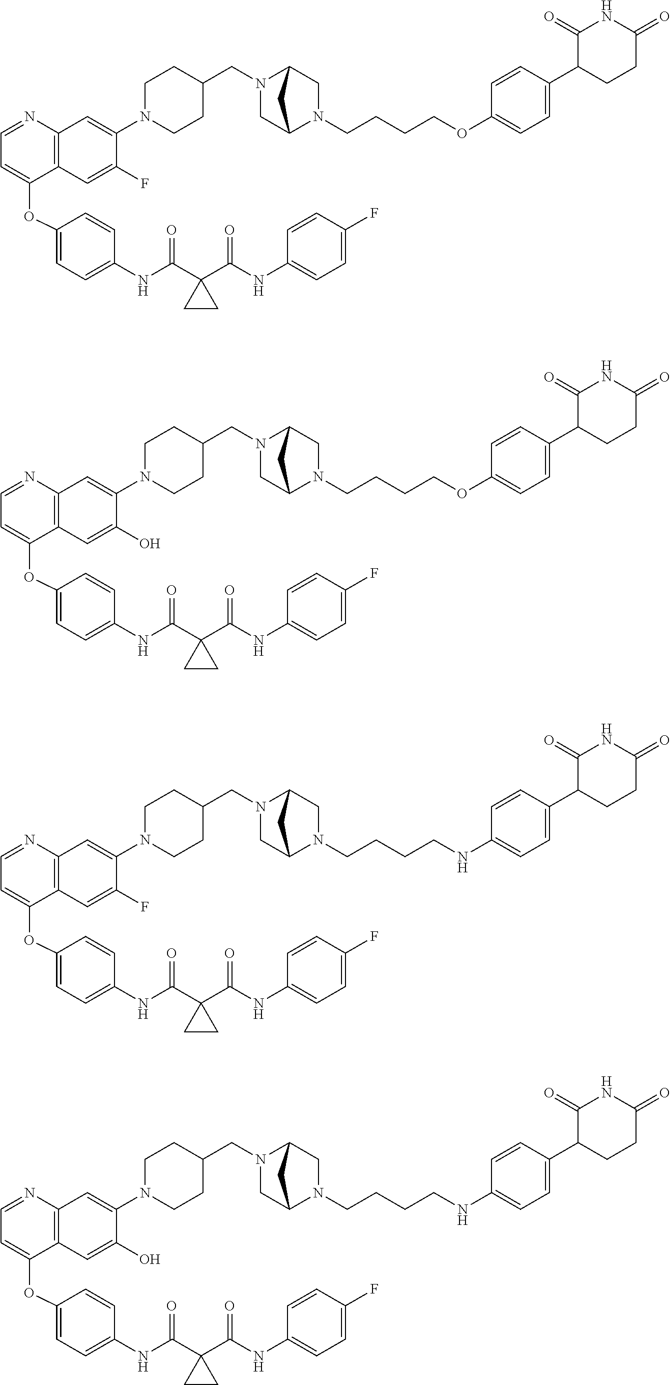 [SMILES: O=C1CCC(c2ccc(OCCCCN3C[C@@H]4C[C@H]3CN4CC3CCN(c4cc5nccc(Oc6ccc(NC(=O)C7(C(=O)Nc8ccc(F)cc8)CC7)cc6)c5cc4F)CC3)cc2)C(=O)N1 ; O=C1CCC(c2ccc(OCCCCN3C[C@@H]4C[C@H]3CN4CC3CCN(c4cc5nccc(Oc6ccc(NC(=O)C7(C(=O)Nc8ccc(F)cc8)CC7)cc6)c5cc4O)CC3)cc2)C(=O)N1 ; O=C1CCC(c2ccc(NCCCCN3C[C@@H]4C[C@H]3CN4CC3CCN(c4cc5nccc(Oc6ccc(NC(=O)C7(C(=O)Nc8ccc(F)cc8)CC7)cc6)c5cc4F)CC3)cc2)C(=O)N1 ; O=C1CCC(c2ccc(NCCCCN3C[C@@H]4C[C@H]3CN4CC3CCN(c4cc5nccc(Oc6ccc(NC(=O)C7(C(=O)Nc8ccc(F)cc8)CC7)cc6)c5cc4O)CC3)cc2)C(=O)N1]

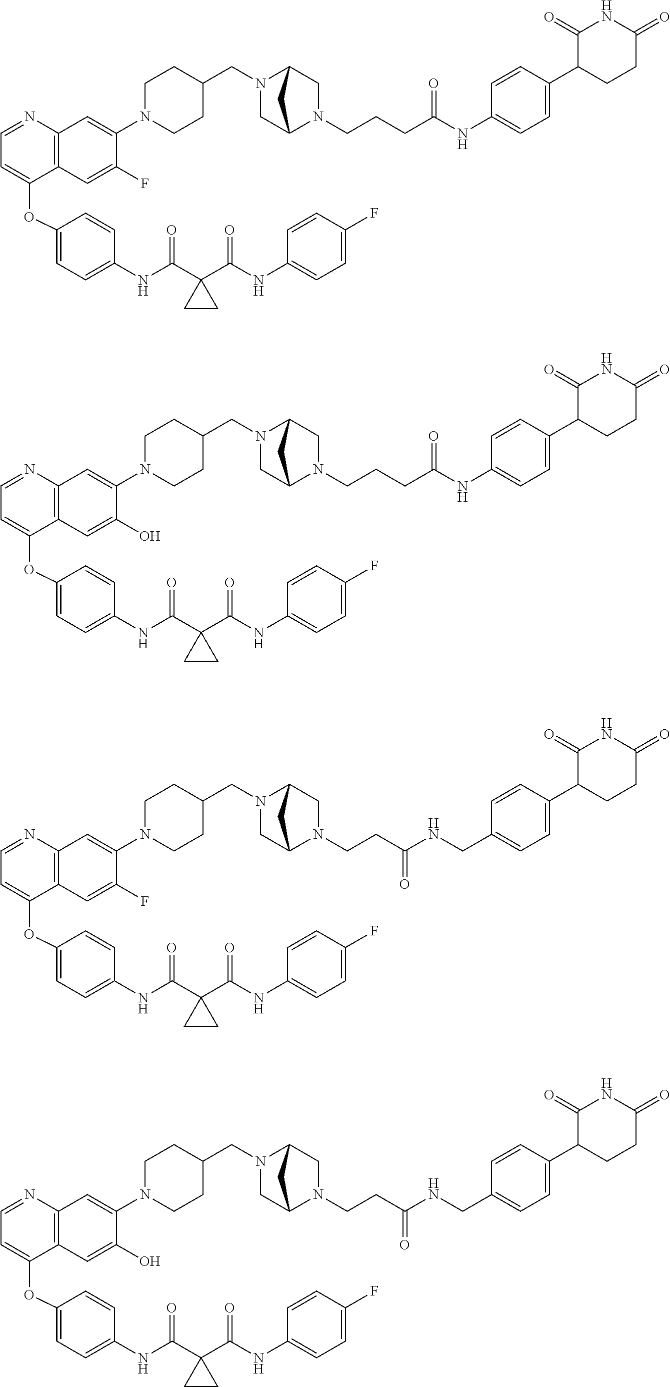

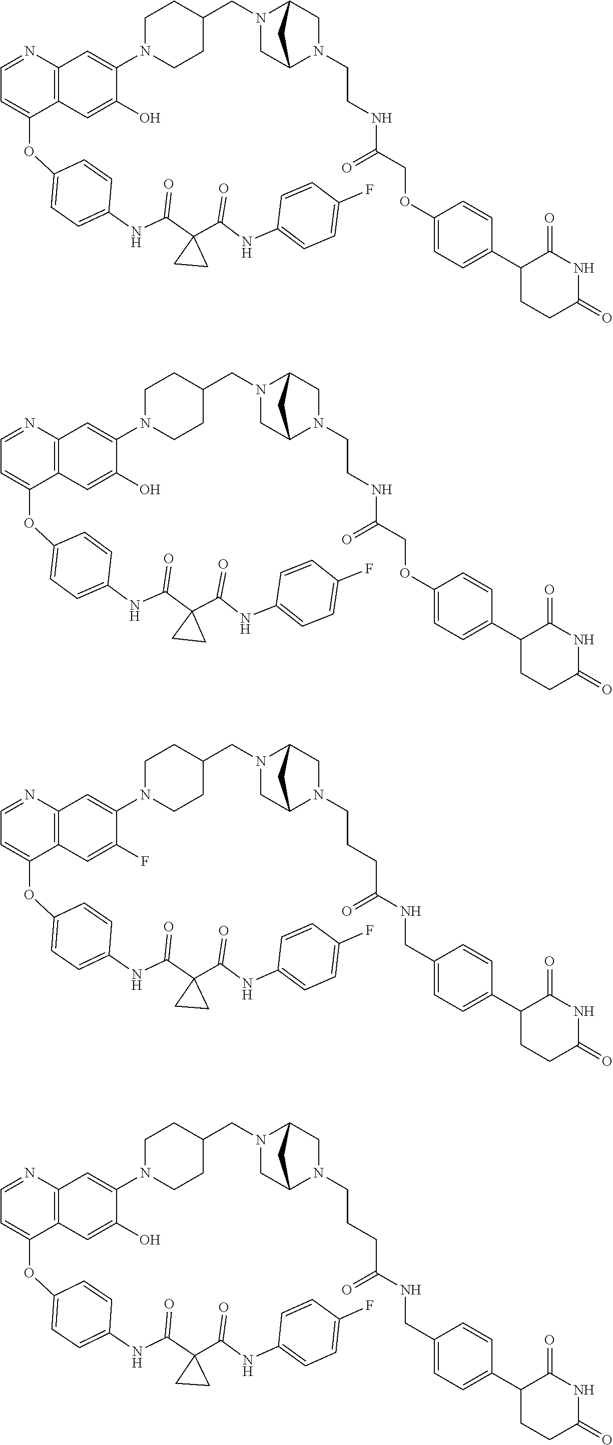

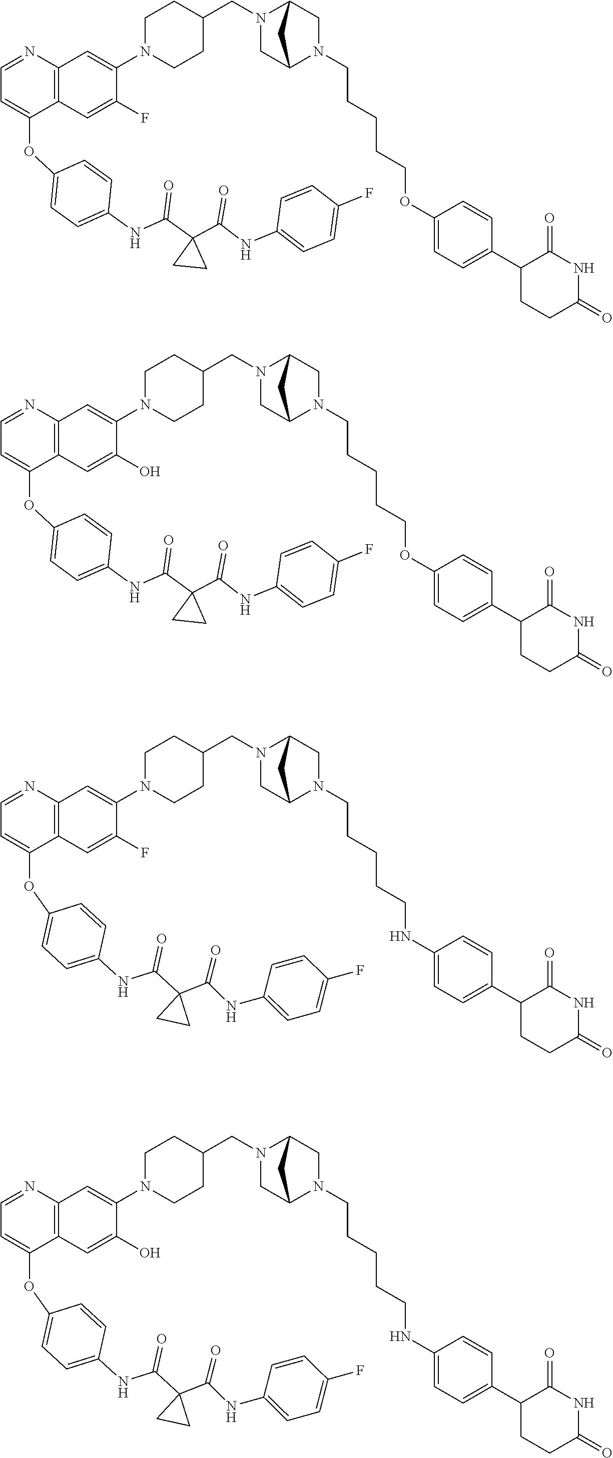 [SMILES: O=C1CCC(c2ccc(OCCCCCN3C[C@@H]4C[C@H]3CN4CC3CCN(c4cc5nccc(Oc6ccc(NC(=O)C7(C(=O)Nc8ccc(F)cc8)CC7)cc6)c5cc4F)CC3)cc2)C(=O)N1 ; O=C1CCC(c2ccc(OCCCCCN3C[C@@H]4C[C@H]3CN4CC3CCN(c4cc5nccc(Oc6ccc(NC(=O)C7(C(=O)Nc8ccc(F)cc8)CC7)cc6)c5cc4O)CC3)cc2)C(=O)N1 ; O=C1CCC(c2ccc(NCCCCCN3C[C@@H]4C[C@H]3CN4CC3CCN(c4cc5nccc(Oc6ccc(NC(=O)C7(C(=O)Nc8ccc(F)cc8)CC7)cc6)c5cc4F)CC3)cc2)C(=O)N1 ; O=C1CCC(c2ccc(NCCCCCN3C[C@@H]4C[C@H]3CN4CC3CCN(c4cc5nccc(Oc6ccc(NC(=O)C7(C(=O)Nc8ccc(F)cc8)CC7)cc6)c5cc4O)CC3)cc2)C(=O)N1]

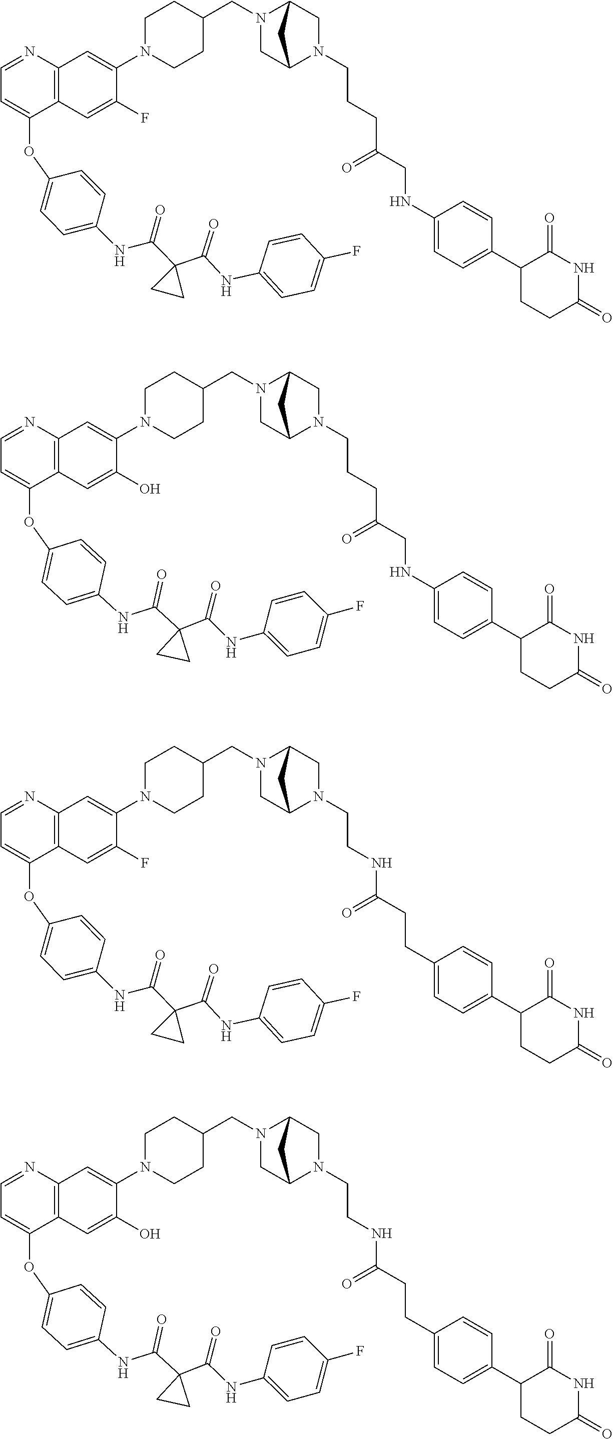

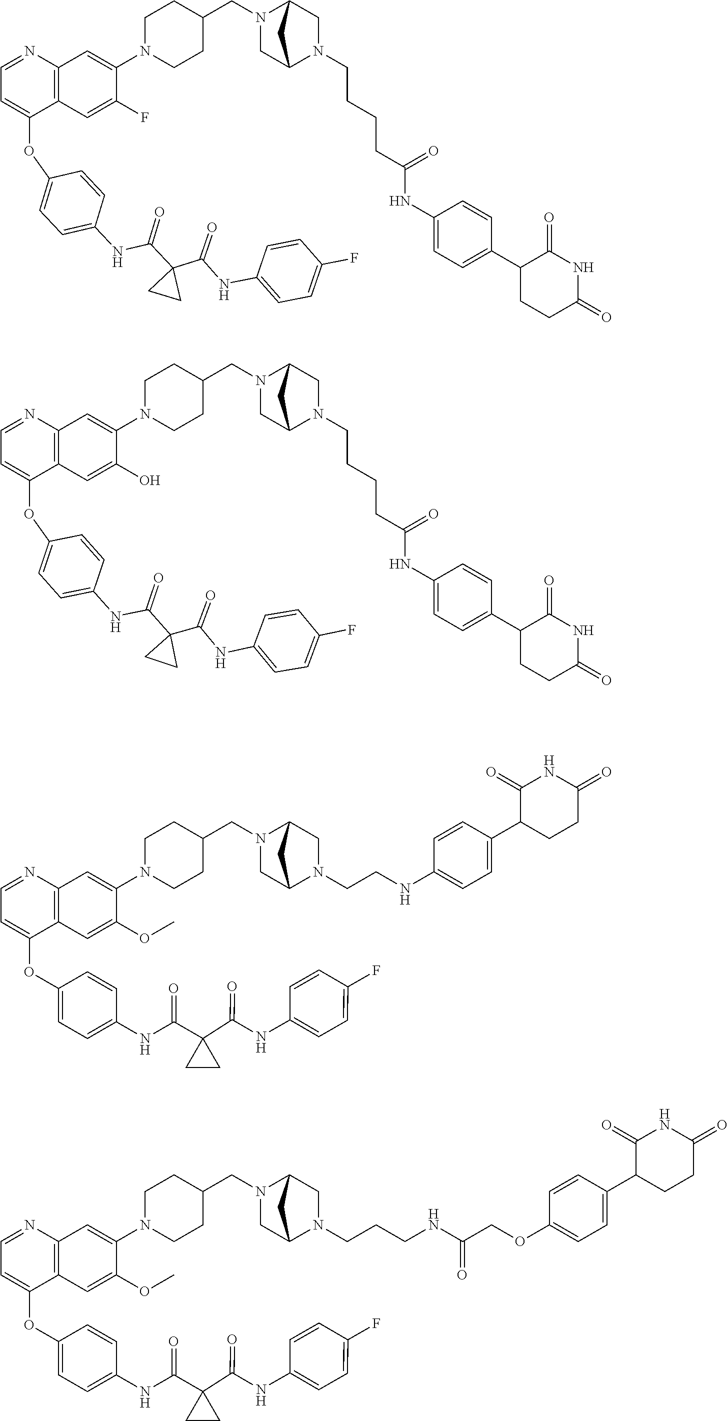

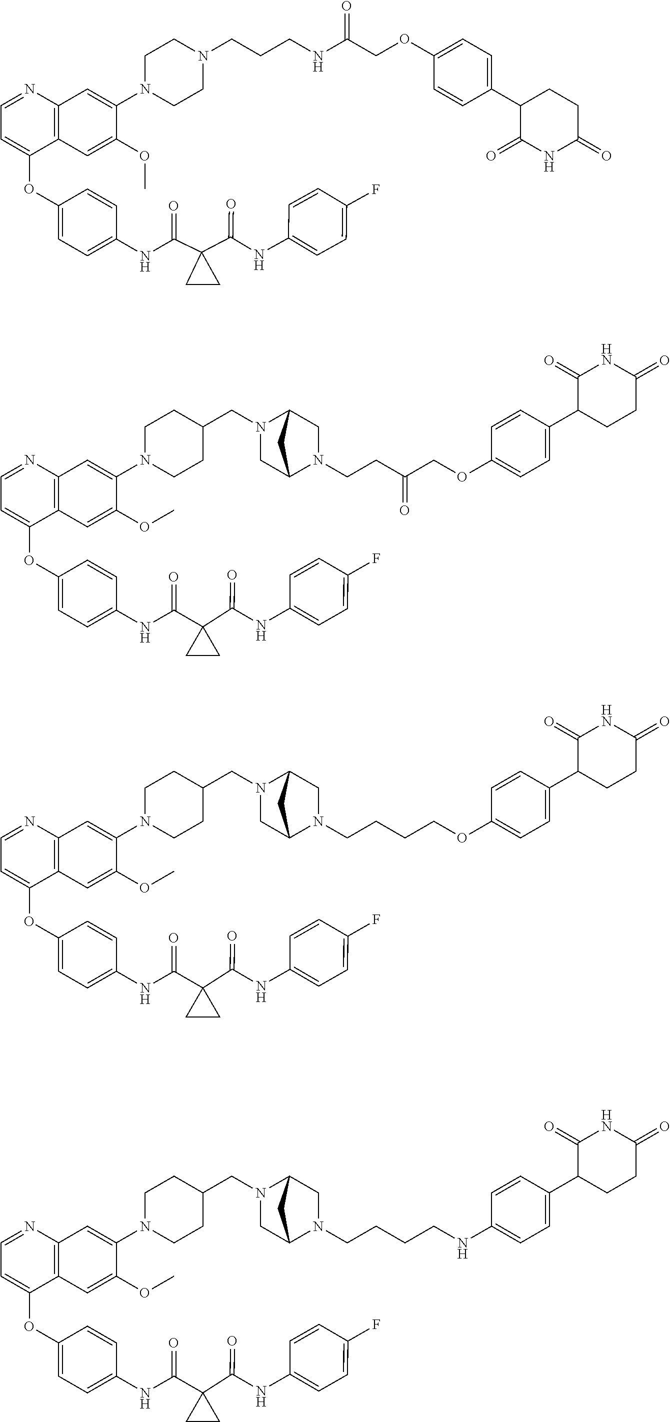 [SMILES: COc1cc2c(Oc3ccc(NC(=O)C4(C(=O)Nc5ccc(F)cc5)CC4)cc3)ccnc2cc1N1CCN(CCCNC(=O)COc2ccc(C3CCC(=O)NC3=O)cc2)CC1 ; COc1cc2c(Oc3ccc(NC(=O)C4(C(=O)Nc5ccc(F)cc5)CC4)cc3)ccnc2cc1N1CCC(CN2C[C@@H]3C[C@H]2CN3CCC(=O)COc2ccc(C3CCC(=O)NC3=O)cc2)CC1 ; COc1cc2c(Oc3ccc(NC(=O)C4(C(=O)Nc5ccc(F)cc5)CC4)cc3)ccnc2cc1N1CCC(CN2C[C@@H]3C[C@H]2CN3CCCCOc2ccc(C3CCC(=O)NC3=O)cc2)CC1 ; COc1cc2c(Oc3ccc(NC(=O)C4(C(=O)Nc5ccc(F)cc5)CC4)cc3)ccnc2cc1N1CCC(CN2C[C@@H]3C[C@H]2CN3CCCCNc2ccc(C3CCC(=O)NC3=O)cc2)CC1]

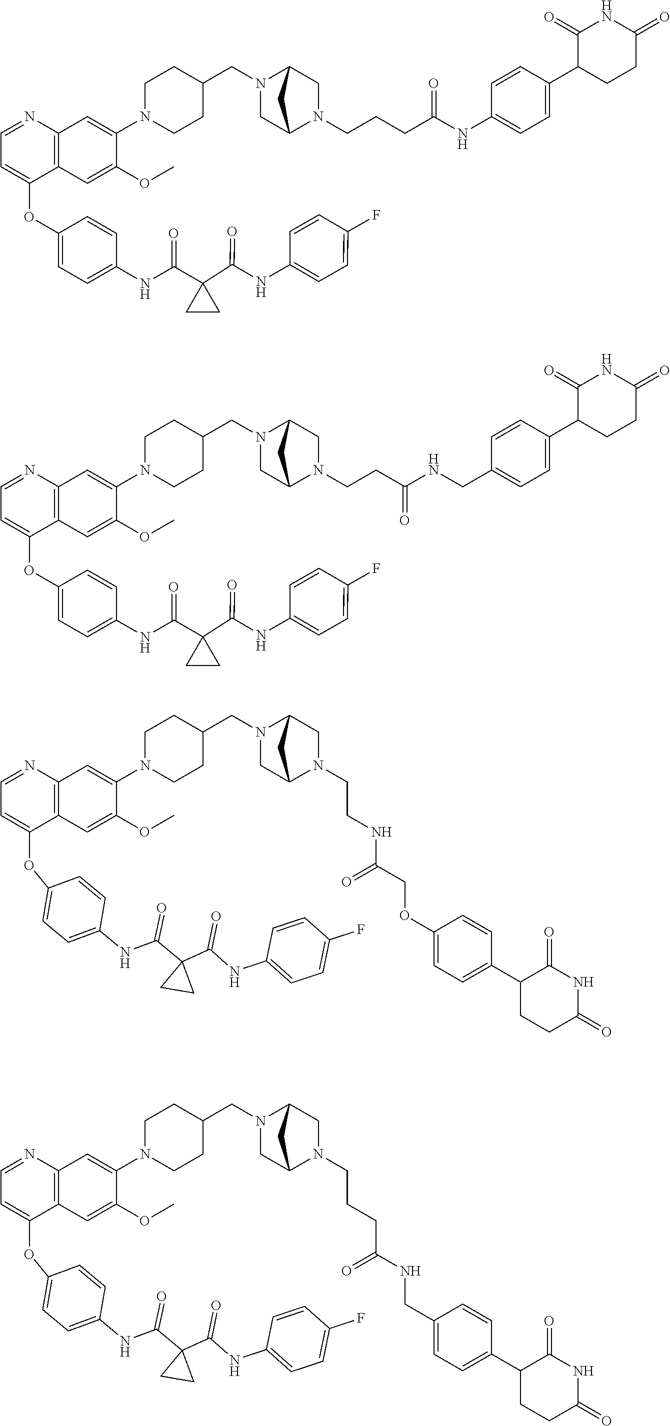

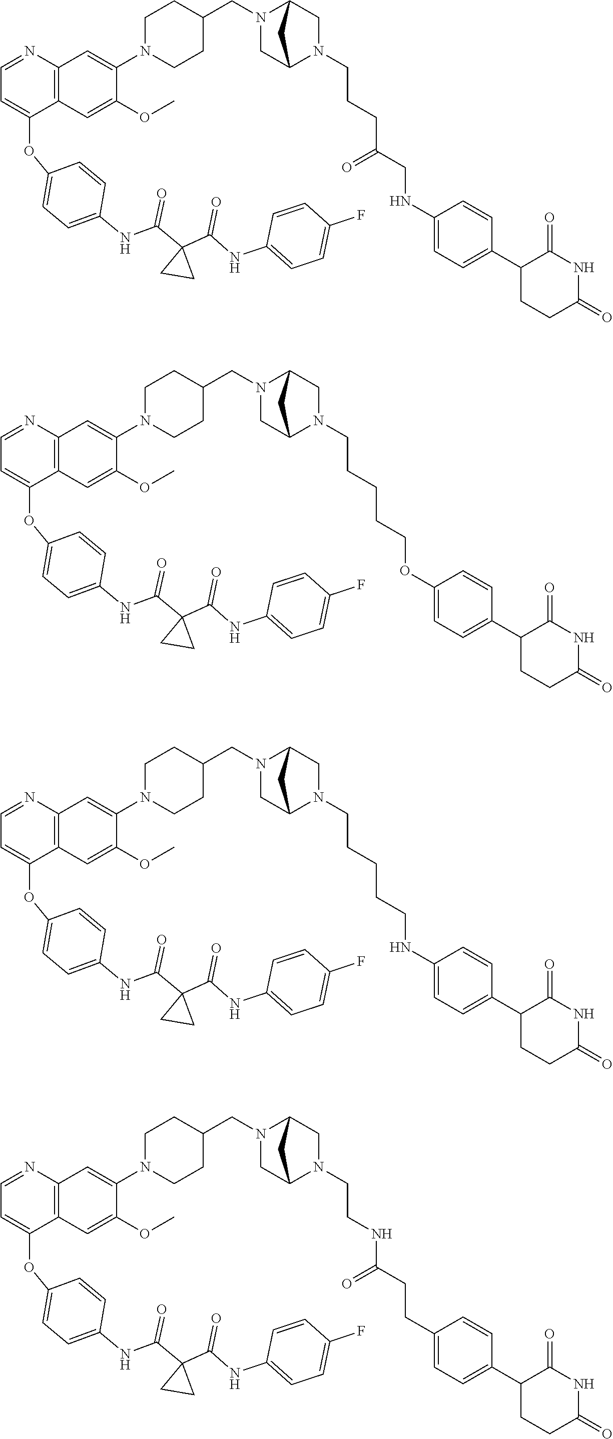 [SMILES: COc1cc2c(Oc3ccc(NC(=O)C4(C(=O)Nc5ccc(F)cc5)CC4)cc3)ccnc2cc1N1CCC(CN2C[C@@H]3C[C@H]2CN3CCCC(=O)CNc2ccc(C3CCC(=O)NC3=O)cc2)CC1 ; COc1cc2c(Oc3ccc(NC(=O)C4(C(=O)Nc5ccc(F)cc5)CC4)cc3)ccnc2cc1N1CCC(CN2C[C@@H]3C[C@H]2CN3CCCCCOc2ccc(C3CCC(=O)NC3=O)cc2)CC1 ; COc1cc2c(Oc3ccc(NC(=O)C4(C(=O)Nc5ccc(F)cc5)CC4)cc3)ccnc2cc1N1CCC(CN2C[C@@H]3C[C@H]2CN3CCCCCNc2ccc(C3CCC(=O)NC3=O)cc2)CC1 ; COc1cc2c(Oc3ccc(NC(=O)C4(C(=O)Nc5ccc(F)cc5)CC4)cc3)ccnc2cc1N1CCC(CN2C[C@@H]3C[C@H]2CN3CCNC(=O)CCc2ccc(C3CCC(=O)NC3=O)cc2)CC1]

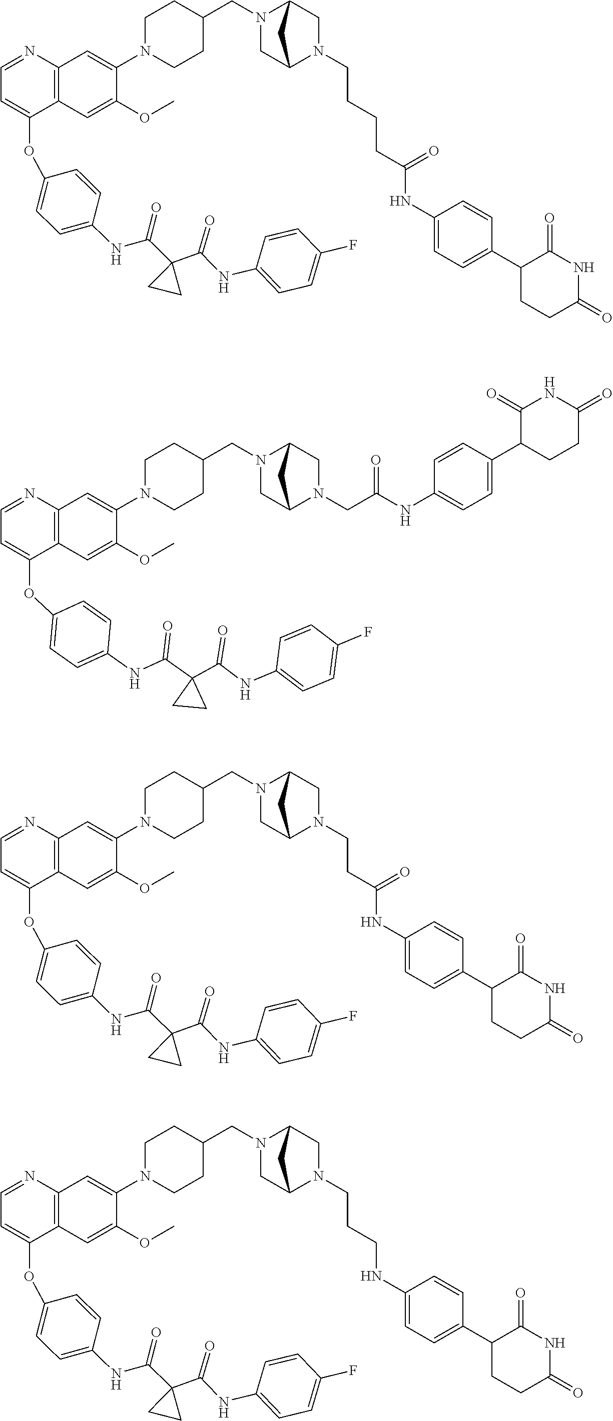 [SMILES: COc1cc2c(Oc3ccc(NC(=O)C4(C(=O)Nc5ccc(F)cc5)CC4)cc3)ccnc2cc1N1CCC(CN2C[C@@H]3C[C@H]2CN3CCCCC(=O)Nc2ccc(C3CCC(=O)NC3=O)cc2)CC1 ; COc1cc2c(Oc3ccc(NC(=O)C4(C(=O)Nc5ccc(F)cc5)CC4)cc3)ccnc2cc1N1CCC(CN2C[C@@H]3C[C@H]2CN3CC(=O)Nc2ccc(C3CCC(=O)NC3=O)cc2)CC1 ; COc1cc2c(Oc3ccc(NC(=O)C4(C(=O)Nc5ccc(F)cc5)CC4)cc3)ccnc2cc1N1CCC(CN2C[C@@H]3C[C@H]2CN3CCC(=O)Nc2ccc(C3CCC(=O)NC3=O)cc2)CC1 ; COc1cc2c(Oc3ccc(NC(=O)C4(C(=O)Nc5ccc(F)cc5)CC4)cc3)ccnc2cc1N1CCC(CN2C[C@@H]3C[C@H]2CN3CCCNc2ccc(C3CCC(=O)NC3=O)cc2)CC1]

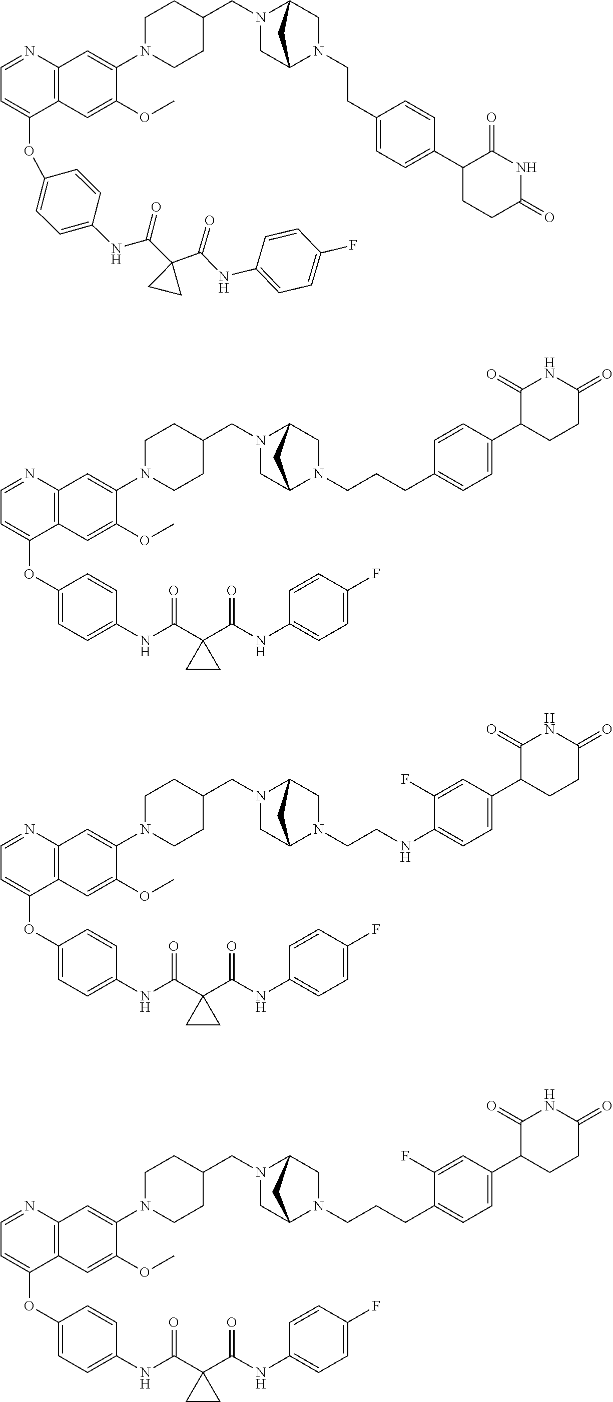 [SMILES: COc1cc2c(Oc3ccc(NC(=O)C4(C(=O)Nc5ccc(F)cc5)CC4)cc3)ccnc2cc1N1CCC(CN2C[C@@H]3C[C@H]2CN3CCc2ccc(C3CCC(=O)NC3=O)cc2)CC1 ; COc1cc2c(Oc3ccc(NC(=O)C4(C(=O)Nc5ccc(F)cc5)CC4)cc3)ccnc2cc1N1CCC(CN2C[C@@H]3C[C@H]2CN3CCCc2ccc(C3CCC(=O)NC3=O)cc2)CC1 ; COc1cc2c(Oc3ccc(NC(=O)C4(C(=O)Nc5ccc(F)cc5)CC4)cc3)ccnc2cc1N1CCC(CN2C[C@@H]3C[C@H]2CN3CCNc2ccc(C3CCC(=O)NC3=O)cc2F)CC1 ; COc1cc2c(Oc3ccc(NC(=O)C4(C(=O)Nc5ccc(F)cc5)CC4)cc3)ccnc2cc1N1CCC(CN2C[C@@H]3C[C@H]2CN3CCCc2ccc(C3CCC(=O)NC3=O)cc2F)CC1]

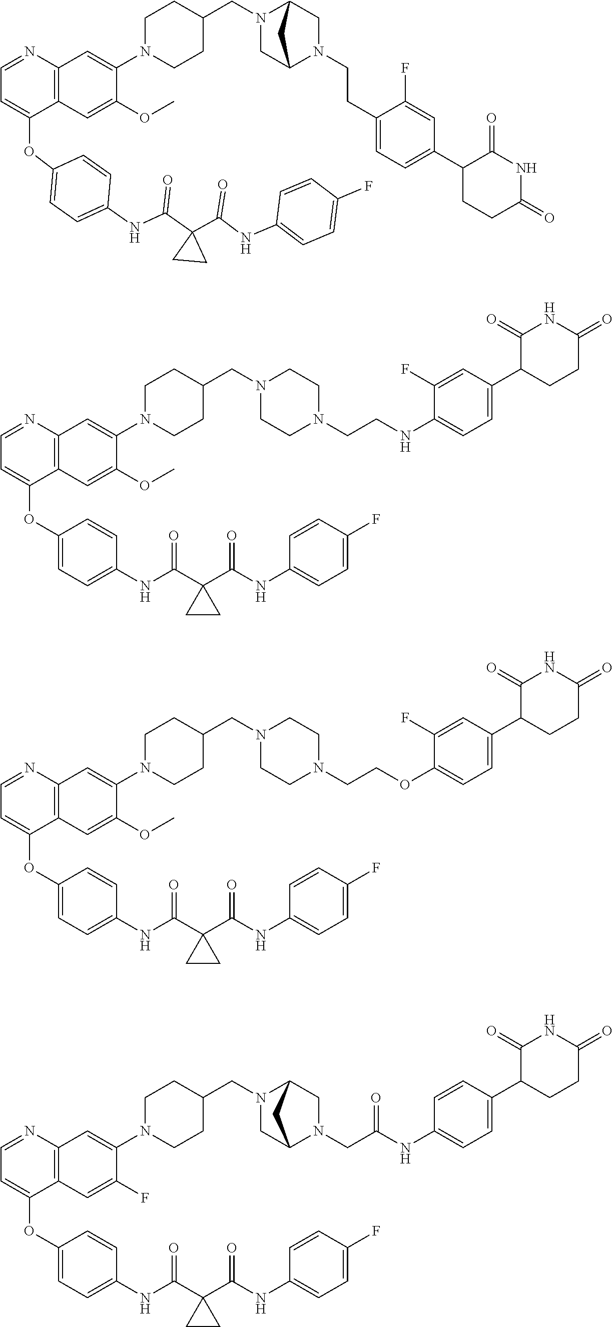 [SMILES: COc1cc2c(Oc3ccc(NC(=O)C4(C(=O)Nc5ccc(F)cc5)CC4)cc3)ccnc2cc1N1CCC(CN2C[C@@H]3C[C@H]2CN3CCc2ccc(C3CCC(=O)NC3=O)cc2F)CC1 ; COc1cc2c(Oc3ccc(NC(=O)C4(C(=O)Nc5ccc(F)cc5)CC4)cc3)ccnc2cc1N1CCC(CN2CCN(CCNc3ccc(C4CCC(=O)NC4=O)cc3F)CC2)CC1 ; COc1cc2c(Oc3ccc(NC(=O)C4(C(=O)Nc5ccc(F)cc5)CC4)cc3)ccnc2cc1N1CCC(CN2CCN(CCOc3ccc(C4CCC(=O)NC4=O)cc3F)CC2)CC1 ; O=C1CCC(c2ccc(NC(=O)CN3C[C@@H]4C[C@H]3CN4CC3CCN(c4cc5nccc(Oc6ccc(NC(=O)C7(C(=O)Nc8ccc(F)cc8)CC7)cc6)c5cc4F)CC3)cc2)C(=O)N1]

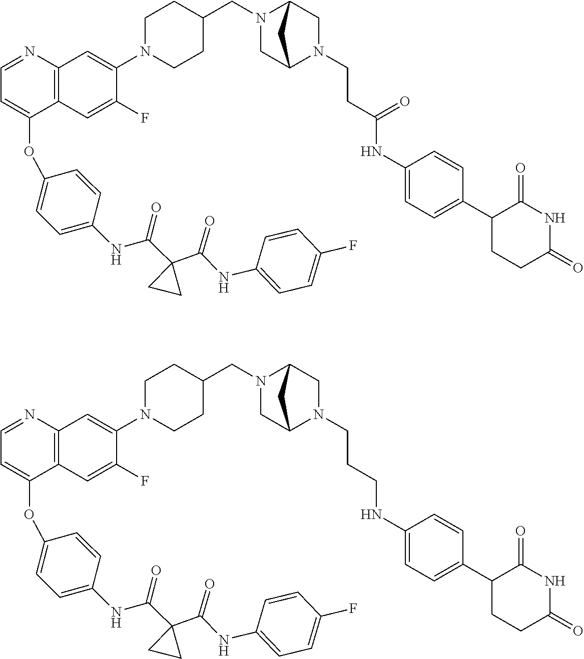 [SMILES: O=C1CCC(c2ccc(NC(=O)CCN3C[C@@H]4C[C@H]3CN4CC3CCN(c4cc5nccc(Oc6ccc(NC(=O)C7(C(=O)Nc8ccc(F)cc8)CC7)cc6)c5cc4F)CC3)cc2)C(=O)N1 ; O=C1CCC(c2ccc(NCCCN3C[C@@H]4C[C@H]3CN4CC3CCN(c4cc5nccc(Oc6ccc(NC(=O)C7(C(=O)Nc8ccc(F)cc8)CC7)cc6)c5cc4F)CC3)cc2)C(=O)N1]

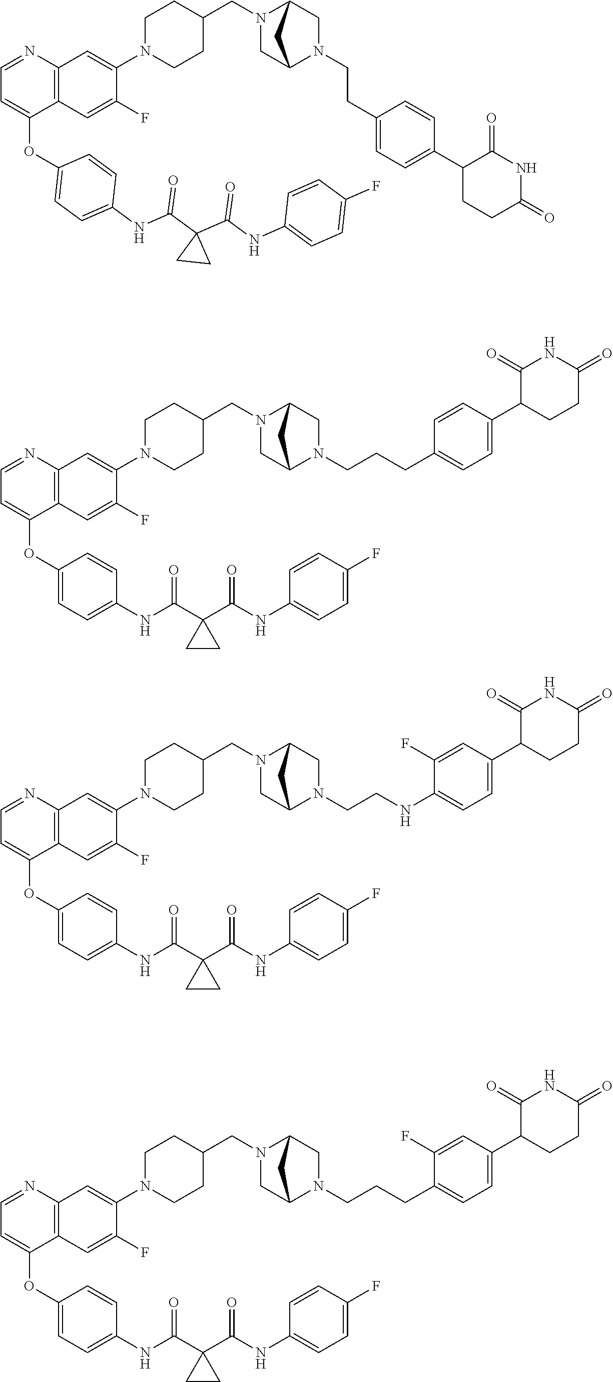 [SMILES: O=C1CCC(c2ccc(CCN3C[C@@H]4C[C@H]3CN4CC3CCN(c4cc5nccc(Oc6ccc(NC(=O)C7(C(=O)Nc8ccc(F)cc8)CC7)cc6)c5cc4F)CC3)cc2)C(=O)N1 ; O=C1CCC(c2ccc(CCCN3C[C@@H]4C[C@H]3CN4CC3CCN(c4cc5nccc(Oc6ccc(NC(=O)C7(C(=O)Nc8ccc(F)cc8)CC7)cc6)c5cc4F)CC3)cc2)C(=O)N1 ; O=C1CCC(c2ccc(NCCN3C[C@@H]4C[C@H]3CN4CC3CCN(c4cc5nccc(Oc6ccc(NC(=O)C7(C(=O)Nc8ccc(F)cc8)CC7)cc6)c5cc4F)CC3)c(F)c2)C(=O)N1 ; O=C1CCC(c2ccc(CCCN3C[C@@H]4C[C@H]3CN4CC3CCN(c4cc5nccc(Oc6ccc(NC(=O)C7(C(=O)Nc8ccc(F)cc8)CC7)cc6)c5cc4F)CC3)c(F)c2)C(=O)N1]

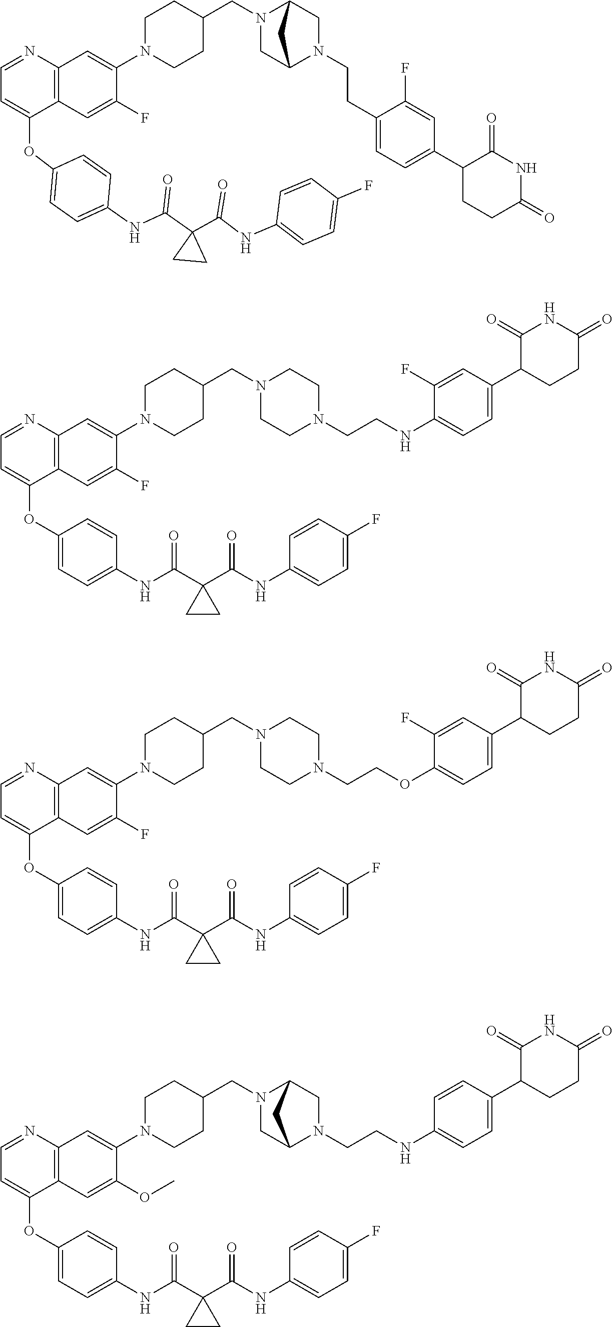 [SMILES: O=C1CCC(c2ccc(CCN3C[C@@H]4C[C@H]3CN4CC3CCN(c4cc5nccc(Oc6ccc(NC(=O)C7(C(=O)Nc8ccc(F)cc8)CC7)cc6)c5cc4F)CC3)c(F)c2)C(=O)N1 ; O=C1CCC(c2ccc(NCCN3CCN(CC4CCN(c5cc6nccc(Oc7ccc(NC(=O)C8(C(=O)Nc9ccc(F)cc9)CC8)cc7)c6cc5F)CC4)CC3)c(F)c2)C(=O)N1 ; O=C1CCC(c2ccc(OCCN3CCN(CC4CCN(c5cc6nccc(Oc7ccc(NC(=O)C8(C(=O)Nc9ccc(F)cc9)CC8)cc7)c6cc5F)CC4)CC3)c(F)c2)C(=O)N1 ; COc1cc2c(Oc3ccc(NC(=O)C4(C(=O)Nc5ccc(F)cc5)CC4)cc3)ccnc2cc1N1CCC(CN2C[C@@H]3C[C@H]2CN3CCNc2ccc(C3CCC(=O)NC3=O)cc2)CC1]

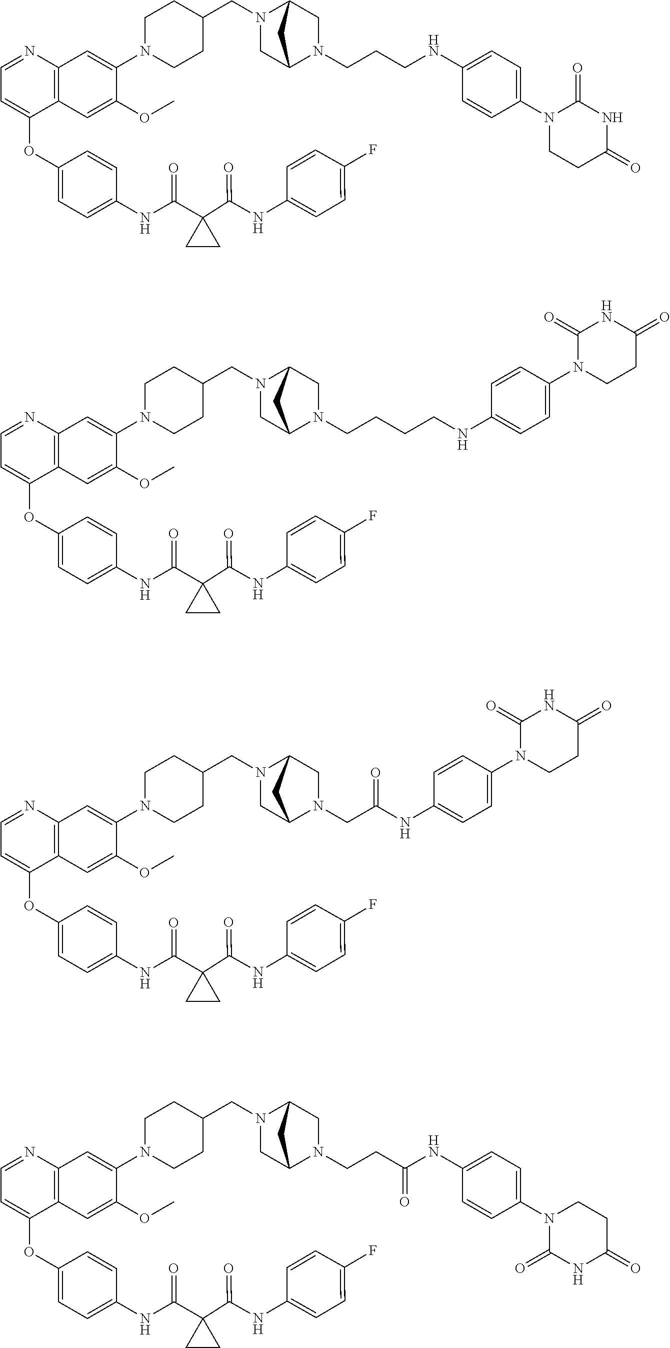 [SMILES: COc1cc2c(Oc3ccc(NC(=O)C4(C(=O)Nc5ccc(F)cc5)CC4)cc3)ccnc2cc1N1CCC(CN2C[C@@H]3C[C@H]2CN3CCCNc2ccc(N3CCC(=O)NC3=O)cc2)CC1 ; COc1cc2c(Oc3ccc(NC(=O)C4(C(=O)Nc5ccc(F)cc5)CC4)cc3)ccnc2cc1N1CCC(CN2C[C@@H]3C[C@H]2CN3CCCCNc2ccc(N3CCC(=O)NC3=O)cc2)CC1 ; COc1cc2c(Oc3ccc(NC(=O)C4(C(=O)Nc5ccc(F)cc5)CC4)cc3)ccnc2cc1N1CCC(CN2C[C@@H]3C[C@H]2CN3CC(=O)Nc2ccc(N3CCC(=O)NC3=O)cc2)CC1 ; COc1cc2c(Oc3ccc(NC(=O)C4(C(=O)Nc5ccc(F)cc5)CC4)cc3)ccnc2cc1N1CCC(CN2C[C@@H]3C[C@H]2CN3CCC(=O)Nc2ccc(N3CCC(=O)NC3=O)cc2)CC1]

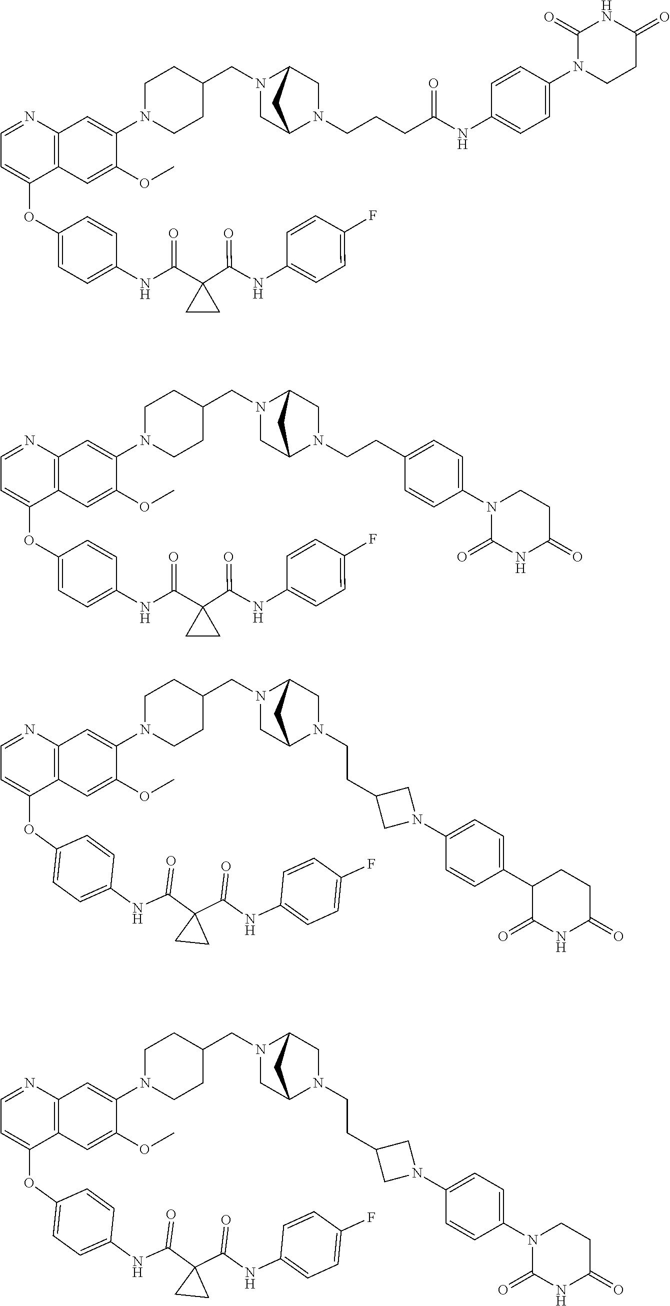

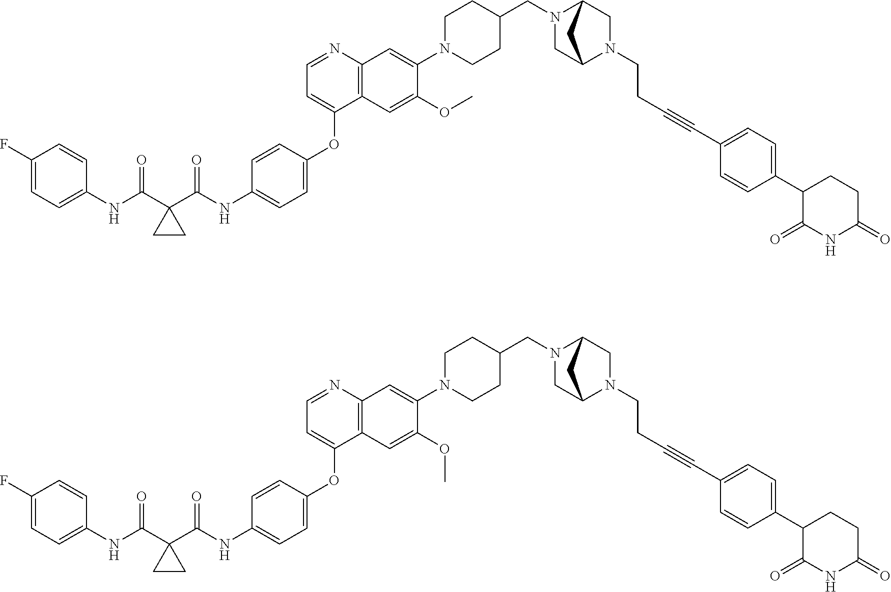

8. A pharmaceutical composition for degrading receptor tyrosine kinase, comprising the bifunctional compound of claim 1 or pharmaceutically acceptable salt, stereoisomer, enantiomer, solvate, polymorph, an isotopic marker, or prodrug thereof.

9. The pharmaceutical composition of claim 8, further comprising at least one bioactivator, wherein
preferably, the bioactivator comprises at least one of an anticancer agent, an immunomodulator, an immune checkpoint inhibitor, a kinase inhibitor, or an anti-inflammatory agent.

10. A method for degradating or inhibiting receptor tyrosine kinase, wherein the method comprises administrating the bifunctional compound of claim 1 to the subject in need.

11. The method of claim 10, wherein the receptor tyrosine kinase comprises a wild-type or locally-mutated discoidin domain receptor (DDR), particularly a DDR1 and/or a DDR2.

12. A method for preventing, diagnosing or treating receptor tyrosine kinase (RTK)-related diseases or symptoms, wherein the method comprises administrating the bifunctional compound of claim 1 to the subject in need.

13. The method of claim 11, wherein the receptor tyrosine kinase (RTK)-related diseases or symptoms comprise cancer associated with abnormal expression of the DDR1 or the DDR2, immune related diseases, fibrotic diseases, neurodegenerative diseases, or inflammatory diseases.

14. The method of claim 13, wherein the cancer comprises gastric cancer, intestinal cancer, esophageal cancer, head and neck cancer, lung cancer, liver cancer, brain cancer, breast cancer, colorectal cancer, skin cancer, thyroid cancer, prostate cancer, soft tissue cancer, endometrial cancer, uterine cancer, testicular cancer, cervical cancer, ovarian cancer, fallopian tube tumor, leukemia, squamous cell cancer, basal cell cancer, adenocarcinoma, renal cell cancer, bladder cancer, kidney cancer, pancreatic cancer, lymphoma, non-Hodgkin lymphoma, melanoma, myeloproliferative disease, sarcoma, angiosarcoma, peripheral neuroepithelioma, glioma, astrocytoma, oligodendroglioma, ependymoma, glioblastoma, neuroblastoma, gangliocytoma, ganglioglioma, medulloblastoma, pineocytoma, meningiomas, meningosarcoma, neurofibroma, or schwannoma.

15. The method of claim 13, wherein the inflammatory diseases and the immune related diseases comprise rheumatoid arthritis, autoimmune encephalomyelitis, ankylosing spondylitis, axial spondyloarthritis, psoriasis, systemic lupus erythematosus, multiple sclerosis, myasthenia gravis, recurrent oral ulcer, kawasaki disease, spondyloarthritis, ophthalmoneuromyelitis, behcet disease, lupus nephritis, familial Mediterranean fever, ulcerative colitis, autoimmune hepatitis, asthma, arteriosclerosis or Crohn disease.

16. The method of claim 13, wherein the neurodegenerative diseases comprise Alzheimer's disease, amyotrophic lateral sclerosis, ataxia telangiectasia, bovine spongiform encephalopathy, Creutzfeldt-Jakob disease, Huntington's chorea, spinocerebellar ataxia, multiple sclerosis, Parkinson's disease, primary lateral sclerosis, spinal muscular atrophy, cerebral ischemia, spastic paraplegia or myasthenia gravis.
